(19) United States
(12) Patent Application Publication
(10) Pub. No.: US 2026/0284009 A1
(43) Pub. Date: Sep. 24, 2026

(54) COMPOUNDS FOR NOS-RELATED CANCERS AND METHODS OF USE

(71) Applicant: YISSUM RESEARCH DEVELOPMENT COMPANY OF THE HEBREW UNIVERSITY OF JERUSALEM LTD., Jerusalem (IL)

(72) Inventor: Haitham AMAL, Haifa (IL)

(73) Assignee: YISSUM RESEARCH DEVELOPMENT COMPANY OF THE HEBREW UNIVERSITY OF JERUSALEM LTD., Jerusalem (IL)

(21) Appl. No.: 19/480,107

(22) PCT Filed: May 10, 2024
(86) PCT No.: PCT/IB2024/054575

§ 371 Date: Oct. 30, 2025

Related U.S. Application Data
(60) Provisional application No. 63/465,524, filed on May 10, 2023.

(51) Int. Cl. A61K 31/416; A61P 35/00

(52) U.S. Cl. CPC A61K 31/416; A61P 35/00

(57) ABSTRACT

Disclosed herein are novel NOS inhibitor compounds. Also disclosed herein, are methods for treating, ameliorating, and/or reducing the severity of NOS-related cancer, or a symptom associated therewith in a subject. The compounds described herein may be administered with a particular route of delivery, dosing regimen, and dosing schedule.

### SEQUENCE LISTING

The present application is being filed along with a Sequence Listing in electronic format. The accompanying Sequence Listing file entitled “40793_0106PTWO-21.xml”, was created on 10 May 2024, which is 16,951 bytes in size. The information in the electronic format of the Sequence Listing is incorporated herein by reference in its entirety.

### BACKGROUND

Nitric oxide (“NO”) is a signaling molecule that plays a significant in physiological function. At low concentrations, NO is a critical signaling molecule, which takes part in the regulation of synaptic activity, synaptic plasticity, and vesicle trafficking. At high concentrations, however, NO can be harmful, resulting in damage to DNA, lipids, and proteins from oxidative stress, modified phenotypes, and cell death. Evidence also suggests that NO may be a key factor in brain-related diseases/disorders, among others. Specifically, research suggests that post-translational nitrosylation of cysteine amino acids may lead to alterations in signaling, which may be responsible for synaptic, neuronal, and behavioral changes in a subject that can lead the cancerous pathologies, discussed herein.

Since NO cannot be stored in the cells, new synthesis is necessary to mediate its activities. In the brain and nervous system, NO is produced by neuronal nitric oxide synthase (“nNOS”), whereas in other parts of the body, e.g., in macrophages, NO is produced by inducible nitric oxide synthase (“iNOS”), and in endothelial cells, NO is produced by endothelial nitric oxide synthase (“eNOS”)—nNOS, iNOS, and eNOS are collectively referred to as the “NOS isoforms.” nNOS is a soluble protein, primarily found in the cytosol of neurons, astrocytes, and adventitia of brain blood vessels. In the nervous system, NO regulates synaptogenesis, vesicle trafficking, neuronal migration, plasticity, and more. Aberrant NO production can cause nitrosative stress by tyrosine nitration of proteins (3-Nitrotyrosine (3-Ntyr)) and by forming S-nitrosoglutathione (GSNO), which is a radical recombination between NO and GSH thiol radicals (RS•); consequently, NO can transform other thiols on peptides and proteins. At higher concentrations, NO can react with superoxide radicals, form peroxynitrite, and induce cell damage. 3-Ntyr is a product of the reaction of proteins with peroxynitrite. 3-Ntyr is an early diagnostic marker of nitrosative stress in neurological disorders. 3-Ntyr can cause disruption of protein native structure and interfere with the phosphorylation ability of tyrosine, and may disrupt many cellular signaling pathways, such as, for example, tyrosine nitration of synaptophysin (Syp), which can lead to cholinergic dysfunction, as well as proteins associated with the mammalian target of rapamycin (“mTOR”), which may be a key mediator of proliferation and survival for glioblastoma and/or neuroblastoma, among others. The free form of 3-Ntyr can be equally toxic.

Moreover, the effects of NO production may depend on NO concentration, exposure time, tumor type, microenvironment, and the like, however, high levels of NO (O(μM)) may cause DNA damage. In recent years, researchers have found that overexpression of NOS isoforms and excessive NO levels may play a role in the development and progression of glioblastoma and/or neuroblastoma, among other diseases, such as, for example, pancreatic cancers, gastrointestinal cancers, sarcomas, ovarian cancers, cervical cancers, lung cancers, melanomas, and cancers of the head and neck and may even hinder the efficacy of conventional therapies used to treat these malignancies.

Without being bound by any particular theory, it is believed that inflammation can induce NO-related carcinogenesis via induction of DNA damage, suppression of DNA repair enzymes, and/or post-translational modifications (“PTMs”) including S-nitrosylation (“SNO”), because during inflammation, reactive nitrogen and oxygen species are released by activated inflammatory cells, attacking neighboring epithelial and stromal cells, altering function, and initiating carcinogenesis. The events leading to the transition to a more aggressive tumor phenotype include activation or upregulation of pro-survival/pro-expansion effector proteins, such as, for example, NF-κB, phosphoinositide-3-kinase (PI3K), AKT, p300, among others.

### SUMMARY

Embodiments of the present disclosure relate to novel NOS inhibitors and formulations thereof, and their use for treating, ameliorating, and/or reducing the severity of a glioblastoma or symptoms associated therewith.

Embodiments of the present disclosure relate to novel NOS inhibitors and formulations thereof, and their use for treating, ameliorating, and/or reducing the severity of a neuroblastoma or symptoms associated therewith.

Embodiments of the present disclosure relate to novel NOS inhibitors and formulations thereof, and their use for treating, ameliorating, and/or reducing the severity of a pancreatic cancer or symptoms associated therewith.

Embodiments of the present disclosure relate to novel NOS inhibitors and formulations thereof, and their use for treating, ameliorating, and/or reducing the severity of a gastrointestinal cancer or symptoms associated therewith.

Embodiments of the present disclosure relate to novel NOS inhibitors and formulations thereof, and their use for treating, ameliorating, and/or reducing the severity of an intestinal adenoma or symptoms associated therewith.

Embodiments of the present disclosure relate to novel NOS inhibitors and formulations thereof, and their use for treating, ameliorating, and/or reducing the severity of a sarcoma or symptoms associated therewith.

Embodiments of the present disclosure relate to novel NOS inhibitors and formulations thereof, and their use for treating, ameliorating, and/or reducing the severity of an ovarian cancer or symptoms associated therewith.

Embodiments of the present disclosure relate to novel NOS inhibitors and formulations thereof, and their use for treating, ameliorating, and/or reducing the severity of a cervical cancer or symptoms associated therewith.

Embodiments of the present disclosure relate to novel NOS inhibitors and formulations thereof, and their use for treating, ameliorating, and/or reducing the severity of a lung cancer associated with an upregulation of NO signaling or symptoms associated therewith.

Embodiments of the present disclosure relate to novel NOS inhibitors and formulations thereof, and their use for treating, ameliorating, and/or reducing the severity of a non-small cell lung cancer or symptoms associated therewith.

Embodiments of the present disclosure relate to novel NOS inhibitors and formulations thereof, and their use for treating, ameliorating, and/or reducing the severity of a metastatic squamous cell carcinoma of the head and neck or symptoms associated therewith.

Embodiments of the present disclosure relate to novel NOS inhibitors and formulations thereof, and their use for treating, ameliorating, and/or reducing the severity of a squamous cell carcinoma of the skin or symptoms associated therewith.

Embodiments of the present disclosure relate to methods for treating, ameliorating, and/or reducing the severity of a glioblastoma or symptoms associated therewith.

Embodiments of the present disclosure relate to methods for treating, ameliorating, and/or reducing the severity of a neuroblastoma or symptoms associated therewith.

Embodiments of the present disclosure relate to methods for treating, ameliorating, and/or reducing the severity of a pancreatic cancer or symptoms associated therewith.

Embodiments of the present disclosure relate to methods for treating, ameliorating, and/or reducing the severity of a gastrointestinal cancer or symptoms associated therewith.

Embodiments of the present disclosure relate to methods for treating, ameliorating, and/or reducing the severity of an intestinal adenoma or symptoms associated therewith.

Embodiments of the present disclosure relate to methods for treating, ameliorating, and/or reducing the severity of a sarcoma or symptoms associated therewith.

Embodiments of the present disclosure relate to methods for treating, ameliorating, and/or reducing the severity of an ovarian cancer or symptoms associated therewith.

Embodiments of the present disclosure relate to methods for treating, ameliorating, and/or reducing the severity of a cervical cancer or symptoms associated therewith.

Embodiments of the present disclosure relate to methods for treating, ameliorating, and/or reducing the severity of a lung cancer associated with an upregulation of NO signaling or symptoms.

Embodiments of the present disclosure relate to methods for treating, ameliorating, and/or reducing the severity of a non-small cell lung cancer or symptoms associated therewith.

Embodiments of the present disclosure relate to methods for treating, ameliorating, and/or reducing the severity of a metastatic squamous cell carcinoma of the head and neck or symptoms associated therewith.

Embodiments of the present disclosure relate to methods for treating, ameliorating, and/or reducing the severity of a squamous cell carcinoma of the skin or symptoms associated therewith.

Embodiments of the present disclosure relate to methods for reducing expression levels of an overexpressed antigen.

Embodiments of the present disclosure relate to methods for reducing expression levels of an overexpressed protein or enzyme.

Embodiments of the present disclosure relate to methods for reducing expression levels of an overexpressed NOS isoform.

Embodiments of the present disclosure relate to methods for selectively reducing expression levels of an overexpressed NOS isoform, without impacting expression levels of other NOS isoforms.

Embodiments of the present disclosure relate to methods for reducing levels of an overexpressed NO-indicator.

Embodiments of the present disclosure relate to methods for providing a larger reduction in tumor volume compared to a subject administered an equivalent dose of a conventional therapy.

Embodiments of the present disclosure relate to methods for providing a larger reduction in tumor mass compared to a subject administered an equivalent dose of a conventional therapy.

Embodiments of the present disclosure relate to methods for providing an improvement in the expected rate of survival of a subject, at, for example, 1 month, 3 months, 6 months, 9 months, 1 year, 2 years, 3 years, 5 years, 10 years, or more, compared to the expected rate of survival of a subject administered an equivalent dose of a conventional therapy.

Embodiments of the present disclosure relate to methods for providing a reduction in the prevalence of side effects resulting from the NOS inhibitor composition compared to the prevalence of side effects observed in a subject administered an equivalent dose of a conventional therapy.

Embodiments of the present disclosure relate to methods for providing a reduction in the severity of side effects resulting from the NOS inhibitor composition compared to the severity of side effects observed in a subject administered an equivalent dose of a conventional therapy.

Embodiments of the present disclosure relate to methods for providing a reduction in the dosage required to achieve an equivalent reduction in tumor volume compared to a subject administered a conventional therapy.

Embodiments of the present disclosure relate to methods for providing a reduction in the dosage required to achieve an equivalent expected rate of survival of a subject compared to a subject administered a conventional therapy.

Embodiments of the present disclosure relate to methods for providing a reduction in the likelihood of a recurrence of the cancer occurring in a subject compared to a subject administered an equivalent dose of a conventional therapy.

Embodiments of the present disclosure relate to methods for providing a reduction in the likelihood of a metastasis occurring in a subject compared to a subject administered an equivalent dose of a conventional therapy.

Embodiments of the present disclosure relate to methods for providing a reduction in the time required to be at a medical facility for treatment compared to a subject administered an equivalent dose of a conventional therapy.

Embodiments of the present disclosure relate to methods for providing a reduction in the cost associated with treatment compared to a subject administered an equivalent dose of a conventional therapy.

Embodiments of the present disclosure relate to methods for restoring/maintaining healthy/optimal expression levels of one or more antigens.

Embodiments of the present disclosure relate to methods for restoring/maintaining healthy/optimal expression levels of one or more protein or enzyme.

Embodiments of the present disclosure relate to methods for restoring/maintaining healthy/optimal expression levels of one or more NOS isoforms.

Embodiments of the present disclosure relate to methods for restoring/maintaining healthy/optimal levels of one or more NO-indicators.

These and other features, aspects, and advantages of the present embodiments of the disclosure will become understood with reference to the following description, appended claims, and accompanying figures.

### BRIEF DESCRIPTION OF THE DRAWINGS

FIG. 1 Dose-response curve of 7-nitroindazole (“7-NI”) against human neuroblastoma (“SH-SY5Y”) cells.

FIGS. 2A-2C depict intervention with 7-NI and/or nNOS-specific siRNA led to a significant decrease in NADPH-diaphorase activity and nitrite levels in SH-SY5Y cells. In FIGS. 2A-2C, for quantitative analysis absorbance at 585 nm was normalized for total protein. Data are expressed as the mean±SEM. Statistical significance was determined using a One-way ANOVA followed by Fisher's LSD multiple comparison post hoc test. *P<0.05 and **P<0.01.

FIG. 2A: Quantitative analysis of NADPH-diaphorase activity in cell lysates of SH-SY5Y cells following the administration of the control (“SH-SY5Y”) (n=3), 0.1% DMSO (“SH-SY5Y+NC”) (n=3), 100 μM 7-NI (“SH-SY5Y+7-NI”) (n=3), or 20 nM siRNA (SEQ ID NO: 4) (“SH-SY5Y+si-nNOS”) (n=3).

FIG. 2B: Quantitative analysis of nitrite levels (a marker of NO levels) measured by Griess assay for SH-SY5Y cells following administration of the control (“SH-SY5Y”) (n=3), 0.1% DMSO (“SH-SY5Y+NC”) (n=3), 100 μM 7-NI (“SH-SY5Y+7-NI”) (n=3), 20 nM siRNA (SEQ ID NO: 4) (“SH-SY5Y+si-nNOS”) (n=3).

FIG. 2C: Representative images of cultured SH-SY5Y cells stained for NADPH-diaphorase activity.

FIGS. 3A-3B depict intervention with 7-NI and/or nNOS-specific siRNA led to a significant decrease in clonogenic survival of SH-SY5Y cells. In FIGS. 3A-3B, for quantitative analysis data, all individual data series are presented as biological replicates (n=4) for all groups. Data are presented as mean±SEM. Statistical significance was determined using a One-way ANOVA followed by Fisher's LSD multiple comparison post hoc test. *P<0.05, **P<0.01, and ns=not significant.

FIG. 3A: Representative well images of clonogenic growth following administration of the control (“SH-SY5Y”), 0.1% DMSO (“SH-SY5Y+NC”), 100 μM 7-NI (“SH-SY5Y+7-NI”), or 20 nM siRNA (SEQ ID NO: 4) (“SH-SY5Y+si-nNOS”).

FIG. 3B: Quantitative analysis of clonogenic survival for the experiments of FIG. 3A.

FIGS. 4A-4D depict intervention with 7-NI and/or nNOS-specific siRNA led to a significant decrease in expression levels of 3-nitrotyrosine (“3-Ntyr”), components of the mTOR signaling pathway, i.e., mammalian target of rapamycin (“mTOR”), its phosphorylated version (“p-mTOR”), tuberous sclerosis complex 2 (“TSC-2”), protein kinase B (“AKT”), its phosphorylated version (“pAKT”), ribosomal protein S6 (“RPS6”), its phosphorylated version (“pRPS6”), and synaptophysin (“Syp”) in SH-SY5Y cells. In FIGS. 4A-4D, for quantitative analysis data, all individual data series are presented as biological replicates (n=4) for all groups. Data were normalized for β-actin and are presented as mean±SEM. Statistical significance was determined using a One-way ANOVA followed by Fisher's LSD multiple comparison post hoc test. *P<0.05 and **P<0.01.

FIG. 4A: Representative western blots of 3-Ntyr prepared from cell lysate of SH-SY5Y cells following administration of the control (“SH-SY5Y”—Series 1), 100 μM 7-NI (“SH-SY5Y +7-NI”—Series 2), or 20 nM siRNA (SEQ ID NO: 4) (“SH-SY5Y+si-nNOS”—Series 3).

FIG. 4B: Quantitative analysis of 3-Ntyr levels for the experiments of FIG. 4A.

FIG. 4C: Representative western blots of mTOR, p-mTOR, TSC-2, AKT, pAKT, RPS6, pRPS6, and Syp prepared from the cell lysate of SH-SY5Y cells following administration of the control (“SH-SY5Y”—Series 1), 100 μM 7-NI (“SH-SY5Y+7-NI”—Series 2), or 20 nM siRNA (SEQ ID NO: 4) (“SH-SY5Y+si-nNOS”—Series 3).

FIG. 4D: Quantitative analysis of mTOR, p-mTOR, TSC-2, AKT, pAKT, RPS6, pRPS6, and Syp levels for the experiments of FIG. 4C.

FIGS. 5A-5C depict intervention with 7-NI and/or nNOS-specific siRNA led to a significant decrease in Syp and 3-Ntyr expression levels in SH-SY5Y cells. In FIGS. 5A-5C, for quantitative analysis each group consisted of three independent sets of experiments run in triplicate. Data are presented as mean±SEM. Statistical significance was determined using a One-way ANOVA followed by Fisher's LSD multiple comparison post hoc test. **P<0.01 and ***P<0.001.

FIG. 5A: Representative immunofluorescent images of SH-SY5Y cells stained for Syp and 3-Ntyr following administration of the control (“SH-SY5Y”), 100 μM 7-NI (“SH-SY5Y+7-NI”), or 20 nM siRNA (SEQ ID NO: 4) (“SH-SY5Y+si-nNOS”). The scale bar is 10 μm.

FIG. 5B: Quantitative analysis of fluorescent intensity of Syp for the experiments of FIG. 5A.

FIG. 5C: Quantitative analysis of fluorescent intensity of 3-Ntyr for the experiments of FIG. 5A.

FIG. 6 depicts intervention with 7-NI led to a significantly smaller tumor volume after 1 month in SH-SY5Y-xenografted mice. FIG. 6 shows quantitative analysis of tumor volume in SH-SY5Y-xenografted mice following administration of the control (n=7) or 80 mg/kg 7-NI (“Treated”) (n=6). Data are presented as mean±SEM. Statistical significance was determined using a One-way ANOVA followed by Fisher's LSD multiple comparison post hoc test. **P<0.01.

FIG. 7 Dose-response curve of 7-NI and N⁶-(1-iminoethyl)-L-lysine (“L-NIL”) against human glioblastoma (“U87-MG”) cells.

FIGS. 8A-8D depict intervention with 7-NI or L-NIL led to a significant decrease in clonogenic survival of U87-MG cells. In FIGS. 8A-8D, for quantitative analysis data, all individual data series are presented as individual technical replicates (n=8) of biological replicates (n=4) for all groups. Data are presented as mean±SEM. Statistical significance was determined using a One-way ANOVA with Bonferroni's multiple comparisons tests. *P<0.05, ***P<0.001, and ns=not significant.

FIG. 8A: Representative well images of clonogenic growth following administration of the control, 0.1% DMSO (“Vehicle”), or 100 μM 7-NI (“7-NI”).

FIG. 8B: Quantitative analysis of clonogenic survival for the experiments of FIG. 8A.

FIG. 8C: Representative well images of clonogenic growth following administration of the control, 500 μM L-NIL (“L-NIL 500”), or 1000 μM L-NIL (“L-NIL 1000”).

FIG. 8D: Quantitative analysis of clonogenic survival for the experiments of FIG. 8C.

FIGS. 9A-9B depict intervention with 7-NI, L-NIL, or combinations thereof led to a significant decrease in clonogenic survival of U87-MG cells. In FIGS. 9A-9B, for quantitative analysis data, all individual data series are presented as individual technical replicates (n=8) of biological replicates (n=4) for all groups. Data are presented as mean±SEM. Statistical significance was determined using a One-way ANOVA with Bonferroni's multiple comparisons tests. *P<0.05, **P<0.01, ***P<0.001, ****P<0.0001, and ns=not significant.

FIG. 9A: Representative well images of clonogenic growth following administration of the control, 0.1% DMSO (“Vehicle”), 100 μM 7-NI (“7-NI”), 500 μM L-NIL (“L-NIL 500”), 1000 μM L-NIL (“L-NIL 1000”), 100 μM 7-NI+500 μM L-NIL (“Combo low”), or 100 μM 7-NI+1000 μM L-NIL (“Combo High”).

FIG. 9B: Quantitative analysis of clonogenic survival for the experiments of FIG. 9A.

FIGS. 10A-10B depict intervention with 7-NI or L-NIL led to a significant decrease in nitrate and nitrite levels in U87-MG cells. In FIGS. 10A-10B, data are presented as mean±SEM. Statistical significance was determined using a One-way ANOVA with Bonferroni's multiple comparisons tests. *P<0.05, **P<0.01, and ns=not significant.

FIG. 10A: Quantitative analysis of nitrate and nitrite levels in U87-MG cells following administration of 0.1% DMSO (“Vehicle”) (n=3) or 100 μM 7-NI (“7-NI”) (n=3).

FIG. 10B: Quantitative analysis of nitrate and nitrite levels in U87-MG cells following administration of control (n=3), 500 μM L-NIL (“L-NIL 500”) (n=3), or 1000 μM L-NIL (“L-NIL 1000”) (n=3).

FIG. 11 depicts intervention with 7-NI, L-NIL, or combinations thereof led to a significant decrease in nitrate and nitrite levels in U87-MG cells. FIG. 11 shows quantitative analysis of nitrate and nitrite levels in U87-MG cells following administration of 0.1% DMSO (“Vehicle”) (n=3), 100 μM 7-NI (“7-NI”) (n=3), 500 μM L-NIL (“L-NIL 500”) (n=3), 1000 μM L-NIL (“L-NIL 1000”) (n=3), 100 μM 7-NI+500 μM L-NIL (“Combo Low”) (n=3), or 100 μM 7-NI+1000 M L-NIL (“Combo High”) (n=3). Data are expressed as the mean±SEM. Statistical significance was determined using a One-way ANOVA with Bonferroni's multiple comparisons tests. *P<0.05 and ns=not significant.

FIGS. 12A-12D depict intervention with 7-NI led to a significant decrease in nitrotyrosine levels, while L-NIL did not result in a significant decrease in 3-Ntyr levels in U87-MG cells. In FIGS. 12A-12D, for quantitative analysis data, all individual data series are presented as biological replicates (n=3) for all groups. Data are expressed as the mean±SEM. Statistical significance was determined using a One-way ANOVA with Bonferroni's multiple comparisons tests. *P<0.05 and ns=not significant.

FIG. 12A: Representative western blots of 3-Ntyr and β-actin levels in in U87-MG cells following administration of the control, 0.1% DMSO (“Vehicle”), or 100 μM 7-NI (“7-NI”).

FIG. 12B: Quantitative analysis of 3-Ntyr levels for the experiments of FIG. 12A.

FIG. 12C: Representative western blots of 3-Ntyr and β-actin levels in in U87-MG cells following administration of the control, 500 μM L-NIL (“L-NIL 500”), or 1000 μM L-NIL (“L-NIL 1000”).

FIG. 12D: Quantitative analysis of 3-Ntyr levels for the experiments of FIG. 12C.

FIGS. 13A-13B depict intervention with 7-NI or L-NIL did not lead to significant changes in cluster of differentiation 70 (“CD-70”) levels in U87-MG cells. In FIGS. 13A-13B, for quantitative analysis data, all individual data series are presented as biological replicates (n=3) for all groups. Data are expressed as the mean±SEM. Statistical significance was determined using a One-way ANOVA with Bonferroni's multiple comparisons tests. ns=not significant.

FIG. 13A: Representative western blots of CD-70 and β-actin levels in in U87-MG cells following administration of the control, 0.1% DMSO (“Vehicle”), 100 μM 7-NI (“7-NI”), 500 μM L-NIL (“L-NIL 500”), or 1000 μM L-NIL (“L-NIL 1000”).

FIG. 13B: Quantitative analysis of CD-70 levels for the experiments of FIG. 13A.

FIGS. 14A-14B depict intervention with 7-NI or L-NIL did not lead to significant changes in inducible nitric oxide synthase (“iNOS”) levels in U87-MG cells. In FIGS. 14A-14B, for quantitative analysis data, all individual data series are presented as biological replicates (n=3) for all groups. Data are expressed as the mean±SEM. Statistical significance was determined using a One-way ANOVA with Bonferroni's multiple comparisons tests. ns=not significant.

FIG. 14A: Representative western blots of iNOS and β-actin levels in in U87-MG cells following administration of the control, 0.1% DMSO (“Vehicle”), 100 μM 7-NI (“7-NI”), 500 μM L-NIL (“L-NIL 500”), or 1000 μM L-NIL (“L-NIL 1000”).

FIG. 14B: Quantitative analysis of iNOS levels for the experiments of FIG. 14A.

FIGS. 15A-15B depict intervention with 7-NI or L-NIL did not lead to significant changes in neuronal nitric oxide synthase (“nNOS”) levels in U87-MG cells. In FIGS. 15A-15B, for quantitative analysis data, all individual data series are presented as biological replicates (n=3) for all groups. Data are expressed as the mean±SEM. Statistical significance was determined using a One-way ANOVA with Bonferroni's multiple comparisons tests. ns=not significant.

FIG. 15A: Representative western blots of nNOS and β-actin levels in in U87-MG cells following administration of the control, 0.1% DMSO (“Vehicle”), 100 μM 7-NI (“7-NI”), 500 μM L-NIL (“L-NIL 500”), or 1000 μM L-NIL (“L-NIL 1000”).

FIG. 15B: Quantitative analysis of nNOS levels for the experiments of FIG. 15A.

FIGS. 16A-16B depict intervention with 7-NI, L-NIL, or the combination thereof led to significantly smaller tumor volume in U87-MG-xenografted mice. In FIGS. 16A-16B, data are expressed as the mean±SEM. Statistical significance was determined using a One-way ANOVA followed by Fisher's LSD multiple comparison post hoc test. **P<0.01, ***P<0.001, ****P<0.0001, and ns=not significant.

FIG. 16A: Temporal tumor volume comparison for U87-MG-xenografted mice receiving treatment with 0.1% DMSO (“vehicle”) (n=10), 80 mg/kg 7-NI (“7-NI”) (n=10), 10 mg/kg L-NIL (“L-NIL”) (n=10), or 80 mg/kg 7-NI and 10 mg/kg L-NIL concurrently administered (“Combo”) (n=15).

FIG. 16B: Quantitative analysis of tumor volume for U87-MG-xenografted mice after 8 days for the experiments of FIG. 16A.

FIGS. 17A-17B depict intervention with 7-NI, L-NIL, or the combination thereof led to significantly smaller tumor volume in a subset of U87-MG-xenografted mice. In FIGS. 17A-17B, data are expressed as the mean±SEM. Statistical significance was determined using a One-way ANOVA followed by Fisher's LSD multiple comparison post hoc test. *P<0.05, ****P<0.0001, and ns=not significant.

FIG. 17A: Photographs of top 4-5 examples (smallest tumor volume) from U87-MG-xenografted mice receiving treatment with 0.1% DMSO (“Vehicle”) (n=10), 80 mg/kg 7-NI (“7-NI”) (n=10), 10 mg/kg L-NIL (“L-NIL”) (n=10), or 80 mg/kg 7-NI and 10 mg/kg L-NIL concurrently administered (“Combo”) (n=15) shown in FIGS. 16A-16B.

FIG. 17B: Quantitative analysis of tumor volume after 8 days for the subset of U87-MG-xenografted mice for the experiments of FIG. 17A.

FIGS. 18A-18B depict intervention with various doses of 7-NI led to significantly smaller tumor volume in U87-MG-xenografted mice. In FIGS. 18A-18B, data are expressed as the mean.

FIG. 18A: Photographs of tumors excised from U87-MG-xenografted mice following intraperitoneal administration of 0.1% DMSO (“vehicle”) (n=8), 20 mg/kg 7-NI (n=8), 40 mg/kg 7-NI (n=8), or 80 mg/kg 7-NI (n=8) for 14 days.

FIG. 18B: Temporal tumor volume comparison for U87-MG-xenografted mice receiving treatment with 0.1% DMSO (“Vehicle”) (n=8), 20 mg/kg 7-NI (n=8), 40 mg/kg 7-NI (n=8), or 80 mg/kg 7-NI (n=8) for 14 days.

FIG. 19 depicts intervention with various doses of 7-NI led to significantly smaller tumor volume in U87-MG-xenografted mice. FIG. 19 shows quantitative analysis of tumor volume for U87-MG-xenografted mice after 14 days for the experiments of FIGS. 18A-18B. Data are expressed as the mean±SEM. Statistical significance was determined using a One-way ANOVA with Bonferroni's multiple comparisons tests. **P<0.01, ****P<0.0001, ns=not significant.

FIG. 20 depicts intervention with various doses of 7-NI led to significantly smaller tumor mass in U87-MG-xenografted mice. FIG. 20 shows quantitative analysis of tumor mass for U87-MG-xenografted mice after 14 days for the experiments of FIGS. 18A-18B. Data are expressed as the mean±SEM. Statistical significance was determined using a One-way ANOVA with Bonferroni's multiple comparisons tests. *P<0.05, **P<0.01, ***P<0.001, and ****P<0.0001.

FIG. 21 depicts intervention with various doses of 7-NI led to increased survival in U87-MG-xenografted mice. FIG. 21 shows a survival plot for U87-MG-xenografted mice receiving treatment with saline (“Vehicle”) (n=10), 80 mg/kg 7-NI (n=10), or 120 mg/kg 7-NI (n=10) daily.

### DETAILED DESCRIPTION

Because cancers may have an unregulated activity of an NOS isoform, namely, nNOS, iNOS, eNOS, or any combination thereof, the inventor has found that inhibition of a specific NOS isoform implicated in a cancer, e.g., one that is abnormally upregulated, can yield a surprisingly efficacious treatment, amelioration, or reduction of severity of that cancer or a symptom associated therewith.

The present inventor has recognized that cancers including those listed above, feature a significant elevation of nitric oxide (NO) (e.g., free, physiologically available NO), which may stem from elevated expression levels of one or more NOS isoforms. The inventor has surprisingly discovered that treatment with NOS inhibitors tailored to suppress a particular NOS isoform's overactivity can lead to a better overall treatment of the specific malignancy.

Herein, for the first time, the inventor reports on the role NO, and the specific NOS isoforms responsible for its production, plays in the body. The inventor reports novel NOS inhibitors and formulations thereof to treat, ameliorate, and/or reduce the severity of various subtypes of cancers including glioblastoma, neuroblastoma, pancreatic cancers, gastrointestinal cancers, intestinal adenomas, sarcomas, ovarian cancers, cervical cancers, lung cancers associated with upregulation of NO signaling, non-small cell lung cancers, metastatic head and neck squamous cell carcinomas, squamous cell carcinomas of the skin, or symptoms associated with any of the foregoing. The inventor has investigated the role of dosage form, dosing regimen, and delivery schedule on efficacy, and report surprising and unexpected results of the novel NOS inhibitors disclosed herein on treating and/or ameliorating the aforementioned cancers or symptoms thereof. To do so, the inventors evaluated the efficacy of the novel NOS inhibitors disclosed herein using in vitro and in vivo in cell-/xenografted-mice models that are representative of the aforementioned cancers. The inventor presents results demonstrating surprising and unexpectedly beneficial efficacy across various physiological metrics relevant to treatment and prognosis of the cancer, such as, for example, cell viability, tumor volume, tumor mass, biomarker expression levels, antigen expression levels, NOS isoform expression levels and/or NO-indicator levels. The results reported herein provide strong evidence that the novel NOS inhibitors disclosed herein may provide significant and unexpected benefits in treating, ameliorating, and/or reducing the severity of various cancers disclosed herein, or symptoms thereof.

Some embodiments provide a composition comprising an amount of one or more NOS inhibitors formulated as a NOS inhibitor composition. As used herein, the term “NOS inhibitors” refers to a class of compounds that inhibit and/or reduce the activity of a NOS enzyme when administered to a subject.

In certain embodiments, “NOS inhibitors” may define a first class of compounds, which includes small molecule organic compounds, and the like, which are collectively referred to herein as “chemical NOS inhibitors.”

In some embodiments, “NOS inhibitors” may define a second class of compounds, which includes, for example, nucleotide-based compounds, e.g., DNA, RNA, and the like, amino acid-based compounds, e.g. oligopeptides, proteins, and the like, genome editing motifs, e.g.

CRISPR/Cas systems, and the like, antibodies, chimeras, viral vectors, and the like, which are collectively referred to herein as “biological NOS inhibitors.”

In some embodiments, a NOS inhibitor composition, as disclosed herein, may comprise a pharmaceutically acceptable vehicle, carrier, or diluent. Some embodiments can be formulated to have varying amounts individual NOS inhibitors within a NOS inhibitor composition, and may define exclusively chemical NOS inhibitors, exclusively biological NOS inhibitors, or a mixture of chemical NOS inhibitors and biological NOS inhibitors.

In some embodiments, an nNOS inhibitor composition, as disclosed herein, may comprise a pharmaceutically acceptable vehicle, carrier, or diluent. Some embodiments can be formulated to have varying amounts individual nNOS inhibitors within an nNOS inhibitor composition, and may define exclusively chemical nNOS inhibitors, exclusively biological nNOS inhibitors, or a mixture of chemical nNOS inhibitors and biological nNOS inhibitors.

In some embodiments, a chemical nNOS inhibitor, as described herein, may be 7-nitroindazole (“7-NI”), or a salt thereof.

In certain embodiments, a chemical NOS inhibitor, as described herein, may be 3-bromo-7-nitroindazole (“3-Br-7-NI”), or a salt thereof.

In some embodiments, a chemical NOS inhibitor, as described herein, may be N^{ω}-propyl-L-arginine (“L-NPA”), or salt thereof, such as, for example, L-NPA hydrochloride.

In certain embodiments, a chemical nNOS inhibitor, as described herein, may be N⁵-(1-iminoethyl)-L-ornithine (“L-NIO”), or a salt thereof, such as, for example, L-NIO dihydrochloride.

In some embodiments, a chemical NOS inhibitor, as described herein, may be N-[4-[2-[[(3-chlorophenyl)methyl]amino]ethyl]phenyl]-2-thiophenecarboximidamide (“ARL-17477”), or a salt thereof, such as, for example, ARL-17477 dihydrochloride.

In certain embodiments, a chemical NOS inhibitor, as described herein, may be N^{ω}-nitro-L-arginine (“L-NNA”), or a salt thereof.

In some embodiments, a chemical NOS inhibitor, as described herein, may be N^{ω}-nitro-L-arginine methyl ester (“L-NAME”), or salt thereof, such as, for example, L-NAME hydrochloride.

In certain embodiments, a chemical nNOS inhibitor, as described herein, may be N^{ω}-methyl-L-arginine (“L-NMMA”), or salt thereof, such as, for example, L-NMMA acetate.

In some embodiments, a chemical NOS inhibitor, as described herein, may be S-[2-[(1-iminoethyl)amino]ethyl]-L-homocysteine (“GW-274150”), or salt thereof, such as, for example, GW-274150 phosphate.

In certain embodiments, a chemical nNOS inhibitor, as described herein, may be N⁶-(1-iminoethyl)-L-lysine (“L-NIL”), or a salt thereof, such as, for example, L-NIL hydrochloride.

In some embodiments, a chemical NOS inhibitor, as described herein, may be N-[[3-(aminomethyl)phenyl]methyl]-ethanimidamide (“1400W”), or a salt thereof, such as, for example, 1400W dihydrochloride.

In certain embodiments, a chemical nNOS inhibitor, as described herein, may be 2,4-disulfophenyl-N-tert-butyl nitrone (“OKN-007”), or a salt thereof, such as, for example, OKN-007 sodium.

In some embodiments, a chemical NOS inhibitor, as described herein, may be 2-[2-(4-methoxypyridin-2-yl)ethyl]-1H-imidazo[4,5-b]pyridine (“BYK-191023”), or a salt thereof, such as, for example, BYK-191023 dihydrochloride.

In certain embodiments, a chemical nNOS inhibitor, as described herein, may be N-{3-[(ethanimidoylamino)methyl]benzyl}-L-prolinamide (“CM544”), or a salt thereof, such as, for example, CM544 dihydrochloride.

In some embodiments, a chemical NOS inhibitor, as described herein, may define a chemical nNOS inhibitor. For purposes of the disclosure, a “chemical nNOS inhibitor” may refer a chemical NOS inhibitor selected from the group consisting of 7-NI, a salt of 7-NI, 3-Br-7-NI, a salt of 3-Br-7-NI, L-NMMA, L-NMMA acetate, L-NPA, L-NPA hydrochloride, L-NNA, L-NAME, L-NAME hydrochloride, L-NIO, L-NIO dihydrochloride, ARL-17477, and/or ARL-17477 dihydrochloride, as well as any salt of the foregoing not expressly listed.

As used herein, the term “chemical nNOS inhibitor,” may refer to a compound that has a preferential affinity for interaction with nNOS compared to the other NOS isoforms, i.e., iNOS and/or eNOS. A “preferential affinity,” as used herein, may be characterized by the compound more strongly inhibiting nNOS than iNOS and/or eNOS, preferably by at least about an order of magnitude. By way of example, the degree of inhibition can be characterized by (i) the compound having a lower dissociation constant for nNOS compared to the dissociation constant(s) for iNOS and/or eNOS, (ii) the compound having a lower inhibitory concentration, such as, for example, an IC50 value, EC50 value, etc., for nNOS compared to the inhibitory concentration(s) for iNOS and/or eNOS, (iii) both (i) and (ii), and (iv) any other metric that would readily be envisaged by one skilled in the art to characterize the ability of a compound to inhibit nNOS, as well as iNOS and/or eNOS. Alternatively, the term “chemical nNOS inhibitor,” may refer to a compound that has a similar affinity for interaction with nNOS compared to the other NOS isoforms, i.e., iNOS and/or eNOS. A “similar affinity,” as used herein, may be characterized by the compound inhibiting nNOS at approximately the same degree as iNOS and/or eNOS, preferably within about the same order of magnitude. By way of example, the degree of inhibition can be characterized by (i) the compound having a similar dissociation constant for nNOS compared to the dissociation constant(s) for iNOS and/or eNOS, (ii) the compound having a similar inhibitory concentration, such as, for example, an IC50 value, EC50 value, etc., for nNOS compared to the inhibitory concentration(s) for iNOS and/or eNOS, (iii) both (i) and (ii), and (iv) any other metric that would readily be envisaged by one skilled in the art to characterize the ability of a compound to inhibit nNOS, as well as iNOS and/or eNOS. Whether “chemical nNOS inhibitor” refers to a “preferential affinity” or a “similar affinity,” as those terms are used herein, will be clear to the skilled artisan based upon the context in which they are used. For example, if a “chemical nNOS inhibitor” was specified as L-NIO, one skilled in the art would immediately envisage that L-NIO has a similar affinity for nNOS compared to the other NOS isoforms, i.e., iNOS and eNOS. Alternatively, for example, if a “chemical nNOS inhibitor” was specified as L-NPA, one skilled in the art would immediately envisage that L-NPA has a preferential affinity for nNOS compared to the other isoforms, i.e., iNOS and eNOS.

In certain embodiments, a chemical NOS inhibitor, as described herein, may define a chemical iNOS inhibitor. For purposes of the disclosure, a “chemical iNOS inhibitor” may refer a chemical NOS inhibitor selected from the group consisting of 7-NI, a salt of 7-NI, 3-Br-7-NI, a salt of 3-Br-7-NI, L-NMMA, L-NMMA acetate, L-NNA, L-NAME, L-NAME hydrochloride, L-NIO, L-NIO dihydrochloride, L-NIL, L-NIL hydrochloride, 1400W, 1400W dihydrochloride, BYK-191023, BYK-191023 dihydrochloride, GW-274150, GW-274150 phosphate, OKN-007, OKN-007 sodium, CM544, and/or CM544 dihydrochloride, as well as any salt of the foregoing not expressly listed.

As used herein, the term “chemical iNOS inhibitor,” may refer to a compound that has a preferential affinity for interaction with iNOS compared to the other NOS isoforms, i.e., nNOS and/or eNOS. A “preferential affinity,” as used herein, may be characterized by the compound more strongly inhibiting iNOS than nNOS and/or eNOS, preferably by at least about an order of magnitude. By way of example, the degree of inhibition can be characterized by (i) the compound having a lower dissociation constant for iNOS compared to the dissociation constant(s) for nNOS and/or eNOS, (ii) the compound having a lower inhibitory concentration, such as, for example, an IC50 value, EC50 value, etc., for iNOS compared to the inhibitory concentration(s) for nNOS and/or eNOS, (iii) both (i) and (ii), and (iv) any other metric that would readily be envisaged by one skilled in the art to characterize the ability of a compound to inhibit iNOS, as well as nNOS and/or eNOS. Alternatively, the term “chemical iNOS inhibitor,” may refer to a compound that has a similar affinity for interaction with iNOS compared to the other NOS isoforms, i.e., nNOS and/or eNOS. A “similar affinity,” as used herein, may be characterized by the compound inhibiting iNOS at approximately the same degree as nNOS and/or eNOS, preferably within about the same order of magnitude. By way of example, the degree of inhibition can be characterized by (i) the compound having a similar dissociation constant for iNOS compared to the dissociation constant(s) for nNOS and/or eNOS, (ii) the compound having a similar inhibitory concentration, such as, for example, an IC50 value, EC50 value, etc., for iNOS compared to the inhibitory concentration(s) for nNOS and/or eNOS, (iii) both (i) and (ii), and (iv) any other metric that would readily be envisaged by one skilled in the art to characterize the ability of a compound to inhibit iNOS, as well as nNOS and/or eNOS. Whether “chemical iNOS inhibitor” refers to a “preferential affinity” or a “similar affinity,” as those terms are used herein, will be clear to the skilled artisan based upon the context in which they are used. For example, if a “chemical iNOS inhibitor” was specified as L-NIO, one skilled in the art would immediately envisage that L-NIO has a similar affinity for iNOS compared to the other NOS isoforms, i.e., nNOS and eNOS. Alternatively, for example, if a “chemical iNOS inhibitor” was specified as 1400W, one skilled in the art would immediately envisage that 1400W has a preferential affinity for iNOS compared to the other isoforms, i.e., nNOS and eNOS.

In some embodiments, a chemical NOS inhibitor, as described herein, may define a chemical eNOS inhibitor. For purposes of the disclosure, a “chemical eNOS inhibitor” may refer a chemical NOS inhibitor selected from the group consisting of 7-NI, a salt of 7-NI, 3-Br-7-NI, a salt of 3-Br-7-NI, L-NMMA, L-NMMA acetate, L-NNA, L-NAME, L-NAME hydrochloride, L-NIO, and/or L-NIO dihydrochloride, as well as any salt of the foregoing not expressly listed.

As used herein, the term “chemical eNOS inhibitor,” may refer to a compound that has a preferential affinity for interaction with eNOS compared to the other NOS isoforms, i.e., nNOS and/or iNOS. A “preferential affinity,” as used herein, may be characterized by the compound more strongly inhibiting eNOS than nNOS and/or iNOS, preferably by at least about an order of magnitude. By way of example, the degree of inhibition can be characterized by (i) the compound having a lower dissociation constant for eNOS compared to the dissociation constant(s) for nNOS and/or iNOS, (ii) the compound having a lower inhibitory concentration, such as, for example, an IC50 value, EC50 value, etc., for eNOS compared to the inhibitory concentration(s) for nNOS and/or iNOS, (iii) both (i) and (ii), and (iv) any other metric that would readily be envisaged by one skilled in the art to characterize the ability of a compound to inhibit eNOS, as well as nNOS and/or iNOS. Alternatively, the term “chemical eNOS inhibitor,” may refer to a compound that has a similar affinity for interaction with eNOS compared to the other NOS isoforms, i.e., nNOS and/or iNOS. A “similar affinity,” as used herein, may be characterized by the compound inhibiting eNOS at approximately the same degree as nNOS and/or iNOS, preferably within about the same order of magnitude. By way of example, the degree of inhibition can be characterized by (i) the compound having a similar dissociation constant for eNOS compared to the dissociation constant(s) for nNOS and/or iNOS, (ii) the compound having a similar inhibitory concentration, such as, for example, an IC50 value, EC50 value, etc., for eNOS compared to the inhibitory concentration(s) for nNOS and/or iNOS, (iii) both (i) and (ii), and (iv) any other metric that would readily be envisaged by one skilled in the art to characterize the ability of a compound to inhibit eNOS, as well as nNOS and/or iNOS. Whether “chemical eNOS inhibitor” refers to a “preferential affinity” or a “similar affinity,” as those terms are used herein, will be clear to the skilled artisan based upon the context in which they are used. For example, if a “chemical eNOS inhibitor” was specified as L-NIO, one skilled in the art would immediately envisage that SMP has a similar affinity for eNOS compared to the other NOS isoforms, i.e., nNOS and iNOS.

In certain embodiments, a biological NOS inhibitor, as described herein, may be a nucleotide-based compound. As used herein, the term “nucleotide-based molecule” refers to RNA duplexes, double-stranded RNAs (“dsRNAs”), silencing, or alternatively short interfering, RNAs (“siRNAs”), small hairpin RNAs (“shRNAs”), microRNAs (“miRNAs”), single stranded RNAs (“ssRNAs”), oligonucleotides, aptamers, mimetics of any of the foregoing, and the like.

Without being bound by any particular theory, it is believed that NOS can be downregulated via RNA silencing. As used herein, the term “RNA silencing” refers to a group of regulatory mechanisms, e.g., RNA interference (“RNAi”), transcriptional gene silencing (“TGS”), post-transcriptional gene silencing (“PTGS”), quelling, co-suppression, translational repression, and the like, that can be mediated by nucleotide-based molecules, as described herein, to a subject. RNA silencing can result in the inhibition, or “silencing,” of the expression of a corresponding protein-coding gene. In some embodiments, RNA silencing, as described herein, may be capable of preventing complete processing, i.e., the full translation and/or expression of an mRNA molecule through a post-transcriptional silencing mechanism. In other embodiments, RNA silencing, as described herein, may be capable of preventing partial processing, i.e., the partial translation and/or expression of an mRNA molecule through a post-transcriptional silencing mechanism. Downregulation of a NOS enzyme can also be achieved by inactivating a gene; for example, nNOS may downregulated by partially or fully inactivating NOS1, iNOS may downregulated by partially or fully inactivating NOS2, eNOS may downregulated by partially or fully inactivating NOS3. Partial or full inactivation of a gene may result from the introduction of targeted mutations involving loss-of function alterations, e.g., point mutations, deletions, and insertions in the gene structure. As used herein, the term “loss-of-function alterations” refers to any mutation in the DNA sequence of a gene, e.g., NOS1, NOS2, and/or NOS3, which results in downregulation of the expression level and/or activity of the expressed product, i.e., the mRNA transcript and/or the translated protein. Non-limiting examples of such loss-of-function alterations include (i) a missense mutation, i.e., a mutation that can change an amino acid residue in the protein, thereby abolishing the enzymatic activity of the protein; (ii) a nonsense mutation, i.e., a mutation that can introduce a stop codon in a protein, which can result in a shorter protein devoid of the enzymatic activity; (iii) a frame-shift mutation, i.e., a mutation, such as a deletion or insertion of nucleic acid(s), that can change the reading frame of the protein, which can result in an early termination, e.g., a truncated protein, devoid of the enzymatic activity, or in a longer amino acid sequence, e.g., a readthrough protein that may have an altered secondary and/or tertiary structure resulting in a non-functional protein, devoid of the enzymatic activity; (iv) a readthrough mutation, which can result from a frame-shift mutation or a modified stop codon mutation and therefore can result in diminished enzymatic activity; (v) a promoter mutation, i.e., a mutation in a promoter sequence, for example, at from the 5′ end to the transcription start site of a gene, which can result in down-regulation of a specific gene product; (vi) a regulatory mutation, i.e., a mutation in a region upstream or downstream of a gene, or within the gene itself, which may affect the expression of the gene product; (vii) a deletion mutation, i.e., a mutation that may result in a deletion of coding nucleic acids in a gene sequence, which may result in a frame-shift mutation or an in-frame mutation, and thus expression; (viii) an insertion mutation, i.e., a mutation which may insert coding or non-coding nucleic acids into a gene sequence, which may result in a frame-shift mutation or an in-frame insertion of one or more amino acid codons, and thus expression; (ix) an inversion, i.e., a mutation that can result in an inverted coding or non-coding sequence; (x) a splice mutation i.e., a mutation which can result in abnormal splicing or poor splicing; and (xi) a duplication mutation, i.e., a mutation which may result in a duplicated coding or non-coding sequence, which can be in-frame or can cause a frame-shift mutation.

In some embodiments, a loss-of-function alteration of a gene, as used herein, may define one more alterations discussed herein of at least one allele of a gene. In certain embodiments, a loss-of-function alteration of a gene, as used herein, may define one more alterations discussed herein of both alleles of a gene. As used herein, the term “allele,” refers to any of one or more alternative forms of a gene locus, wherein alleles of a gene locus relate to and/or define a trait or characteristic. In certain embodiments, a loss-of-function alteration of a gene, as used herein, may comprises both alleles of the gene. In such instances, the gene, e.g., NOS1, NOS2, and/or NOS3, may be in a homozygous form or in a heterozygous form. As used herein, being in a “homozygous form,” refers to a condition where both alleles at the gene's locus are identical. As used herein, being in a “heterozygous form, refers to a condition where both alleles at the gene's locus are different.

In some embodiments, one or more nucleotide-based molecules, as described herein, may comprise dsRNA. In certain embodiments, dsRNA may comprise more than about 30 base pairs (“bp”). By providing dsRNA comprising more than about 30 bp, a sufficient level of gene expression silencing may be observed without inducing a stress response or causing significant off-target effects in a subject receiving the dsRNA. In some embodiments, dsRNA, as described herein, may be provided to affect cells where the interferon pathway is inactive, and may be designed specifically to avoid activating the interferon and protein kinase R (“PKR”) pathways in cells. By way of example, this may be achieved by omitting one or both of a 5′-cap structure and a 3′-poly(A) tail from the dsRNA sequence, to facilitate export of the dsRNA to the cytoplasm, upon entry into a cell, thereby circumventing activation of the interferon or PKR pathways.

Without being bound by any particular theory, it is believed that siRNAs can enter the RNA-induced silencing complex (“RISC”), which can mediate cleavage of single-stranded RNA having sequence complementary to the antisense strand of the siRNA duplex provided thereto. This cleavage of the target RNA can result in RNA silencing, as described herein. It is believed that long strands of dsRNAs, when in cells, can stimulate the activity of a ribonuclease III enzyme referred to as “Dicer.” Dicer may be involved in the processing of the dsRNA into siRNAs. siRNAs derived from Dicer can be about 21 to about 23 nucleotides in length, comprising about 19 bp duplexes, which can improve the rate or efficiency of entry of the siRNA duplex into RISC, thereby improving the effect of RNA silencing.

In certain embodiments, one or more nucleotide-based molecules, as described herein, may comprise siRNA. In some embodiments, siRNA may comprise RNA duplexes of about 18 bp to about 30 bp, more preferably about 25 bp to about 30 bp. In certain embodiments, siRNAs, as described herein may comprise asymmetric duplexes having a 3′-overhang on the antisense strand. By way of example, siRNAs, as disclosed herein, may be, for example, any siRNA disclosed in any of SEQ ID NOs: 3-10.

In some embodiments, siRNAs, as described herein, may be constructed to form a hairpin or stem-loop structure, i.e., a shRNA. As used herein, the term “shRNA,” refers to an RNA compound having a stem-loop structure, comprising a first and second region of complementary sequence, the degree of complementarity and orientation of the regions being sufficient such that base pairing occurs between the regions, the first and second regions being joined by a loop region, the loop resulting from a lack of base pairing between nucleotides (or nucleotide analogs) within the loop region. In certain embodiments, the number of nucleotides in the loop is a number between about 3 to about 23, preferably about 5 to about 15, more preferably about 7 to about 13, or most preferably about 9 to about 11. In some embodiments, nucleotides in the loop can be involved in base-pair interactions with other nucleotides in the loop. By way of example, shRNAs, as disclosed herein, may be, for example, any shRNA disclosed in any of SEQ ID NOs: 11-16. Examples of oligonucleotide sequences that can be used to form the loop include, but are not limited to 5′-CAAGAGA-3′ (SEQ ID NO: 1) and 5′-UUACAA-3′ (SEQ ID NO: 2).

In certain embodiments, one or more nucleotide-based molecules, as described herein, may comprise miRNA. In some embodiments, miRNAs may comprise single stranded RNA having a length of about 19 to about 28 nucleotides. For purposes of the disclosure, suitable miRNA sequences defining nucleotide-based molecules, include, but are not limited to miR-146a, hsa-miR-146a-5p (MIRT735124), hsa-miR-1273f (MIRT783312), hsa-miR-143-3p (MIRT783811), hsa-miR-147a (MIRT783856), hsa-miR-3622a-5p (MIRT784976), hsa-miR-3911 (MIRT785428), hsa-miR-4269 (MIRT785560), hsa-miR-4708-5p (MIRT786482), hsa-miR-4710 (MIRT786499), hsa-miR-4770 (MIRT786696), hsa-miR-4792 (MIRT786788), hsa-miR-574-5p (MIRT787873), hsa-miR-6088 (MIRT788038), hsa-miR-644a (MIRT788184), hsa-miR-6715b-5p (MIRT788450), hsa-miR-6768-5p (MIRT788631), hsa-miR-6867-5p (MIRT789213), hsa-miR-7855-5p (MIRT789601), hsa-miR-8485 (MIRT789865), as well as any synthetic version of the foregoing.

In some embodiments, one or more nucleotide-based molecules, as described herein, may comprise a miRNA mimetic. As used herein, the term “miRNA mimic” refers to synthetic non-coding RNAs. In certain embodiments, miRNA mimics imitate the function of endogenous miRNAs and can be designed as mature, double stranded molecules, or mimic precursors, e.g., pre-miRNAs. In some embodiments, miRNA mimics can be comprised of modified or unmodified RNA, DNA, RNA-DNA hybrids, or alternative nucleic acid chemistries, e.g., locked nucleic acids (“LNAs”) or 2′-O,4′-C-ethylene-bridged nucleic acids (“BNAs”). When miRNA mimetics are designed as mature, double stranded miRNA mimics, the length of the duplex region may be between about 13 to about 33, preferably about 18 to about 24, or more preferably about 21 to about 23 nucleotides. Generally, miRNA mimetics may comprise a total of about at least 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39 or 40 nucleotides.

In certain embodiments, one or more nucleotide-based molecules, as described herein, may comprise a ssRNA.

In some embodiments, one or more nucleotide-based molecules, as described herein, may comprise an oligonucleotide.

In certain embodiments, one or more nucleotide-based molecules, as described herein, may comprise an aptamer. As used herein, the term “aptamer” refers to a dsRNA or ssRNA designed to target a specific molecular target, e.g., a protein.

In some embodiments, modified nucleotide-based molecules, including modified and/or synthetic DNA or RNA constituents, may be used in the in place of naturally occurring nucleic acid counterparts in the nucleotide-based molecules described herein. In certain embodiments, substituting one or more modified and/or synthetic nucleic acids in a nucleotide-based molecule can improve the half-life, stability, specificity, delivery, solubility, and nuclease resistance of the nucleotide-based molecules described herein. By way of example, an siRNA may partially or entirely comprise synthetic nucleotide analogs that confer the beneficial qualities described above. As used herein, the term “modified nucleotide-based molecules,” defines molecules in which one or more of the components of a nucleic acid, i.e., namely sugars, bases, and phosphate moieties, are different from that which occurs in nature, and preferably different from that which occurs in the human body.

The modifications described herein can be incorporated into any dsRNA, siRNA, shRNA, miRNA, RNA-like, DNA, DNA-like, or any mimetic of the foregoing. In some embodiments, it may be desirable to modify one or both of the antisense and sense strands of a nucleotide-based molecule. In certain embodiments, it may be desirable to incorporate the modifications described herein at a 3′ or 5′ terminal position, or in the last 2, 3, 4, 5, or 10 nucleotides of a strand. In some embodiments, the sense stand and antisense strand may have the same modifications or the same class of modifications, but in other cases the sense and antisense strand can have different modifications, e.g., in some cases it may be desirable to modify only one strand, e.g., the sense strand. In some embodiments, a modification to the backbone of the nucleotide-based molecule may be incorporated, for example, a modification of a sugar of the nucleotide-based molecule or a modification of a nucleobase of the nucleotide-based molecule. In certain embodiments, a modification, as described herein, may define modifying a linkage between consecutive nucleotide bases and/or the general backbone structure of the nucleotide-based molecule. For purposes of the disclosure, modifications may include, but are not limited to a phosphate-ribose backbone or linker, a phosphate-deoxyribose backbone or linker, a phosphorothioate-deoxyribosebackbone or linker, a 2′-O-methyl-phosphorothioate backbone or linker, a phosphorodiamidate morpholino backbone or linker, a peptide nucleic acid backbone or linker, a 2-methoxyethyl phosphorothioate backbone or linker, an alternating locked nucleic acid backbone or linker, a constrained ethyl backbone or linker, a phosphorothioate backbone or linker, a N3′-P5′ phosphoroamidate backbone or linker, a 2′-deoxy-2′-fluoro-β-d-arabino nucleic acid backbone or linker, a cyclohexene nucleic acid backbone or linker, a tricyclo-DNA (tcDNA) backbone or linker, a ligand-conjugated antisense backbone or linker, any combination thereof, and the like.

Modifications, as described herein, may be incorporated in a nucleotide-based molecule to provide an improved property of the resulting nucleotide-based molecule. For purposes of the disclosure, an improved property, as described herein, may be one or more of (i) an increase in the intracellular stability of the nucleotide-based molecule; (ii) an increase in in vivo stability of the nucleotide-based molecule, e.g., an increased resistance to proteases; (iii) an increase in cellular uptake of the nucleotide-based molecule; (iv) an improvement in the binding affinity of the nucleotide-based molecule; (v) an increase in one or more pharmacokinetic parameters of the nucleotide-based molecule; (vi) a resistance to polymerase extension from the 3′ end of the nucleotide-based molecule; (vii) a resistance to recognition from a polymerase of the nucleotide-based molecule; (viii) a resistance to double-strand triggered degradation of the nucleotide-based molecule; and (ix) a resistance to RISC-mediated degradation, or any parallel nucleic acid degradation pathway, of the nucleotide-based molecule.

Synthesis of the one or more nucleotide-based molecules disclosed herein may be accomplished by known methods of the art, and may be, for example, chemical synthesis or recombinant synthesis.

In certain embodiments, one or more nucleotide-based molecules, as described herein, may display specific targeting of nNOS, without cross inhibiting other NOS enzymes, i.e., iNOS or eNOS, or a splice variant which exhibits 99% or less global homology to the target gene, e.g., less than 98%, 97%, 96%, 95%, 94%, 93%, 92%, 91%, 90%, 89%, 88%, 87%, 86%, 85%, 84%, 83%, 82%, 81% global homology to the target gene; as determined by PCR, western blot, immunohistochemistry and/or flow cytometry.

In some embodiments, one or more nucleotide-based molecules, as described herein, may display specific targeting of iNOS, without cross inhibiting other NOS enzymes, i.e., nNOS or eNOS, or a splice variant which exhibits 99% or less global homology to the target gene, e.g., less than 98%, 97%, 96%, 95%, 94%, 93%, 92%, 91%, 90%, 89%, 88%, 87%, 86%, 85%, 84%, 83%, 82%, 81% global homology to the target gene; as determined by PCR, western blot, immunohistochemistry and/or flow cytometry.

In certain embodiments, one or more nucleotide-based molecules, as described herein, may display specific targeting of eNOS, without cross inhibiting other NOS enzymes, i.e., nNOS or iNOS, or a splice variant which exhibits 99% or less global homology to the target gene, e.g., less than 98%, 97%, 96%, 95%, 94%, 93%, 92%, 91%, 90%, 89%, 88%, 87%, 86%, 85%, 84%, 83%, 82%, 81% global homology to the target gene; as determined by PCR, western blot, immunohistochemistry and/or flow cytometry.

In some embodiments, one or more nucleotide-based molecules, as described herein, may be delivered to a subject using a viral delivery system. In certain embodiments, the viral delivery system may be viral vector (“VV”) defining an adeno-associated virus (“AAV”), such as, for example, serotype 9 (“AAV9”), a lentivirus, a herpes simplex virus, an oncoretrovirus, and the like. In some embodiments, VVs may be formulated to be able to cross the blood brain barrier of the subject. In some embodiments, VVs comprising nucleotide-based molecules may be administered alone or may be administered in combination with one or more other VVs, e.g., a second VV encoding for or having one or more different transgenes. In certain embodiments, VVs may be suspended in a physiologically compatible carrier, including, but not limited to saline or a buffered version thereof, lactose, sucrose, calcium phosphate, gelatin, dextran, agar, pectin, peanut oil, sesame oil, water, and the like. In some embodiments, VVs may further comprise one or more conventional pharmaceutical ingredients, such as stabilizers or preservatives, including, but not limited to chlorobutanol, potassium sorbate, sorbic acid, sulfur dioxide, propyl gallate, parabens, ethyl vanillin, glycerin, phenol, parachlorophenol, gelatin, albumin, and the like. In certain embodiments, a viral delivery system, as described herein, may be formulated to target a specific tissue or organ of a subject, including, but not limited to the anus, the ascending colon, the base of the tongue, a blood vessel, the bile duct, the brainstem, the cecum, the cerebellum, the cerebral cortex, the cervix, the dermis, the descending colon, the duodenum, the epidermis, the epiglottis, the esophagus/gastroesophageal junction, a fallopian tube, an area of fat, the gallbladder, the gastrointestinal stroma, the glottis, the hard palate, the hypodermis, the hypopharynx, the hypothalamus, the ileum, the jejunum, the larynx, a lining of a joint, the lips, the liver, a lung, the meninges, a muscle, the nasal cavity, the nasopharynx, a nerve, the oral cavity, the oropharynx, an ovary, the pancreas, the paranasal sinuses, the pineal gland, the pituitary gland, the rectum, the renal system, the salivary glands, the soft palate, the sigmoid colon, the spinal cord, the stomach, the subglottis, the supraglottis, a tendon, the thalamus, the tongue, one or more tonsils, or the transverse colon.

The dose of VVs required to achieve a desired effect or “therapeutic effect,” e.g., the units of dose in vector genomes/per kilogram of body weight (vg/kg), can vary based on several factors including, but not limited to the route of VV administration, the level of gene or RNA expression required to achieve a therapeutic effect, the specific disease or disorder being treated, the stability of the gene or RNA product, and the pharmacokinetic, bioavailability, or bioaccumulation properties of the VV. One of skill in the art can readily determine an VV virion dose range to treat a subject having a particular disease or disorder based on the aforementioned factors, as well as other factors that are well known in the art. By way of example, an effective amount of the VV, as described herein, may be within the range of about 10 μL to about 100 ml of solution containing from about 10⁹ to 10¹⁶ genome copies per subject, though the volume of solution used is not particularly limited, and can depend on the size of the subject, the dose of the VV, the route of administration, among others. For example, for intrathecal or intracerebral administration, a volume in the range of about 1 μL to about 100 μL may be used, whereas for intravenous administration, a volume in the range of about 10 μL to about 10 mL may be used. In some cases, a dosage between about 10¹⁰ to about 10⁵ VV genome copies per subject may be appropriate, and in certain cases the above dosage may be normalized to a subject's weight, i.e., 10¹⁰ to about 10¹⁵ VV genome copies per kg.

In certain embodiments, a biological NOS inhibitor, as described herein, may be a genome editing motif. As used herein, the term “genome editing motif” refers to meganucleases, zinc finger nucleases (“ZFNs”), transcription activator-like effector nucleases (“TALENs”), CRISPR/Cas systems, transposases, recombinant adeno-associated viruses, mimetics of any of the foregoing, and the like.

Meganucleases, as described herein, may define an enzyme within one of the LAGLIDADG family, the GIY-YIG family, the His-Cys box family, and the HNH family. In some embodiments, meganucleases may comprise an enzyme that has a recognition sequence of about 12 bp to about 40 bp. In certain embodiments, meganucleases may comprises one or more mutations from their naturally occurring counterpart. In some embodiments, one or more mutations, as described herein may be incorporated through mutagenesis, and the like. Mutagenesis, as described herein, refers to consistent exposure to a mutagen, which may be, for example a genetic mutation, a chemical, a radiation agent, and the like. For example, the mutagen may be ionizing radiation, such as, ultraviolet light, gamma rays, alpha particles, and the like. Other mutagens may include, for example, (i) base analogs, which can cause copying errors during DNA replication; (ii) deaminating agents, e.g., nitrous acid; (iii) intercalating agents, e.g., ethidium bromide; (iv) alkylating agents, e.g., bromouracil; (v) transposons; (vi) alkaloids; (vii) bromine or compounds thereof; (viii) sodium azide; (ix) psoralen, e.g., combined with ultraviolet radiation; (x) chemical agents, e.g., ICR191, 1,2,7,8-diepoxy-octane (“DEO”), 5-azaC,N-methyl-N-nitrosoguanidine (“MNNG”), ethyl methane sulfonate (“EMS”), and the like, as well as any combination thereof. The mutagens disclosed herein are not particularly limited, and would be readily envisaged by one skilled in the art.

ZFNs and TALENs, as described herein, may define two distinct classes of engineered nucleases designed to effectively produce targeted double-stranded breaks. In some embodiments, ZFNs and TALENs may utilize a non-specific DNA cutting enzyme that is linked to a specific DNA binding domain, i.e., a zinc finger domains for ZFNs and TALE repeat domains for TALENs. In certain embodiments, the non-specific ZFNs and TALENs, as described herein, may comprise the enzyme Fok1. When present, Fok1 may be engineered to function exclusively as a heterodimer, which may increase catalytic activity and specificity. In some embodiments, ZFNs and TALENs, as described herein, allow for deletions of about 5 bp to about 500 bp of DNA.

Approaches for making ZFNs, as described herein, may include but are not limited to modular assembly (where Zinc fingers correlated with a triplet sequence are attached in a row to cover the required sequence), OPEN (low-stringency selection of peptide domains vs. triplet nucleotides followed by high-stringency selections of peptide combination vs. the final target in bacterial systems), and bacterial one-hybrid screening of zinc finger libraries, among others.

Approaches for making TALENs, as described herein, may include methods known in the field, such as the methods described in Reyon et al., Nat. Biotechnol., 2012, 30(5):460-465, Miller et al., Nat Biotechnol., 2011, 29: 143-148, Cermak et al., Nucl. Acids Res., 2011, 39 (12): e82, and Zhang et al., Nat. Biotechnol., 2011, 29(2): 149-153, all of which are hereby incorporated by reference in their entirety.

CRISPR/Cas systems, as described herein, may define a gRNA and an endonuclease, that when provided together, can degrade nucleic acids via double-stranded breaks. In some embodiments, a gRNA, as described herein, may comprise a 20-nucleotide sequence encoding a combination of the target homologous sequence (crRNA) and the endogenous bacterial RNA that links the crRNA to an endonuclease in a single chimeric transcript (tracrRNA). In certain embodiments, an endonuclease, as described herein, may be selected from the group consisting of Cas1, Cas2, Cas3, Cas4, Cas5, Cas6, Cas7, Cas8a, Cas8b, Cas8c, Cas9, Cas10, Cas10d, Cas12, Cas12a, Cas12b, Cas12c, Cas12d, Cas12e, Cas12f, Cas12g, Cas12h, Cas12i, Cas12k, C2c4, C2c8, C2c9, Cas13, Cas13a, Cas13b, Cas13c, Cas13d, Cas13x.1, Cse1, Cse2, Csy1, Csy2, Csy3, GSU0054, Csm2, Cmr5, Csx10, Csx11, Csf1, Csn2, and the like. The specific gRNA sequence and endonuclease component would be readily envisioned by one having skill the art. In some embodiments, CRISPR/Cas systems, as described herein, may be designed to target multiple genes simultaneously.

Transposases, as described herein, may define an enzyme that binds to the ends of a transposon and catalyzes the movement of the transposon to another part of the genome. As used herein, the term “transposon” refers to a mobile genetic element comprising a nucleotide sequence, that can be displaced move around to different positions within a genome, which may cause mutations and/or change the amount of a DNA in the genome of the cell. Examples of transposases, include, but are not limited to Sleeping Beauty, piggyBac, and the like. In some embodiments, transposases may induce transposition via a “cut-and-paste” mechanism, wherein terminal repeats, such as TTAA, may be transposed into other locations of the genome.

Recombinant adeno-associated viruses (“rAAVs”), as described herein, may define recombinantly made VVs that can enable insertion, deletion, or substitution of DNA sequences in a genome. In certain embodiments, the genome of an rAAV may be a single-stranded deoxyribonucleic acid (“ssDNA”) molecule, either positive- or negative-sensed, which may be about 4000 bp to about 5000 bp long. In some embodiments, rAAVs may target a single allele and/or may prevent any off-target genomic alteration. One of skill in the art can design a rAAV vector, according to methods known in the art.

In certain embodiments, a biological NOS inhibitor, as described herein, may be an antibody. As used herein, the term “antibody” refers to monoclonal antibodies (“mAbs”), polyclonal antibodies (“PAbs”), bivalent antibodies, mimetics of any of the foregoing, fragments of any of the foregoing, and the like. In some embodiments, antibodies, as described herein, may be designed to specifically bind nNOS, iNOS, and/or eNOS, and may therefore be capable of downregulating the respective NOS isoform. In some embodiments, the specificity for binding a NOS isoform may result from antibody binding of an nNOS-, iNOS-, or eNOS epitope through the conserved, or constant, (“Fc”) region of the antibody. In certain embodiments, the specificity for binding nNOS may result from antibody binding of an nNOS-, iNOS-, or eNOS epitope through the variable fragment (“Fv”) region of the antibody. In some embodiments, the Fv region of the antibody can be designed to provide an antibody with a specific binding affinity for one or more epitopes of NOS. Exemplary antibodies include, but are not limited to 37-2800 from ThermoFisher, as well as EP1855Y and EPR 24351-6 from abeam.

Methods of producing antibodies, as described herein, can be accomplished utilizing methods that are well-known in the art, such that those described by Harlow and Lane in Antibodies: A Laboratory Manual, Cold Spring Harbor Laboratory, New York, 1988, which is hereby incorporated by reference in its entirety.

In certain embodiments, a biological NOS inhibitor, as described herein, may be a chimera. As used herein, the term “chimera” refers to a proteolysis-targeting chimera, such as PROTAC. In some embodiments, a PROTAC may be heterobifunctional—comprising one ligand that can bind to a ubiquitin ligase, e.g., E3 ubiquitin ligase, a second ligand that can bind to nNOS, iNOS, or eNOS, and optionally a linker connecting the two ligands. In certain embodiments, when a PROTAC, as described herein, binds to a target protein, an exposed lysine can be ubiquitinated, which may recruit the ubiquitin proteasome system (“UPS”), and may thereby inducing UPS-mediated protein degradation.

In certain embodiments, a biological NOS inhibitor, as described herein, may be an amino acid-based compound. As used herein, the term “amino acid-based compound” refers to peptides, oligopeptides, proteins, mimetics of any of the foregoing, and the like. In some embodiments, an amino acid-based compound can bind at least a catalytic or binding portion of nNOS, which can downregulate nNOS. In certain embodiments, an amino acid-based compound can bind at least a catalytic or binding portion of iNOS, which can downregulate iNOS. In some embodiments, an amino acid-based compound can bind at least a catalytic or binding portion of eNOS, which can downregulate eNOS. In certain embodiments, an amino acid-based compound may be a PIN protein or a nitric oxide synthase interacting protein (NOSIP). In some embodiments, an amino acid-based compound may be designed to bind an active site of nNOS, wherein the active site is selected from the group consisting of Phenylalanine 589, Tryptophan 592, Tyrosine 593, Glutamate 597, Aspartate 602, Arginine 601, Methionine 341, Histidine 342, Tyrosine711.

In some embodiments, an NOS inhibitor comprising one or more nNOS inhibitors composition, as described herein, may be formulated to be able to cross the blood brain barrier of a subject. By providing at least one or more compounds that cross the blood brain barrier of a subject, the NOS inhibitor composition a more robust patient outcome, i.e., better treatment of a disease, condition, or disorder and/or the amelioration and/or reduction in the severity of symptoms associated therewith can be realized, wherein the disease, conditions, or disorder is glioblastoma and/or neuroblastoma. Without being bound by any particular theory, it is believed that nNOS, an endogenous protein, which is responsible for production of NO, is predominantly expressed in the brain, and therefore causes an increase in brain NO levels. The increase of NO in the brain may be responsible for the emergence of the glioblastomas and/or neuroblastomas disclosed herein, or the symptoms associated therewith. Thus, by providing compounds that can cross the blood brain barrier of a subject, the compounds can more systemically inhibit nNOS activity, and consequently the levels of NO in the brain and the abnormal pathologies that occur therefrom.

In some embodiments, a NOS inhibitor composition comprising one or more chemical NOS inhibitors, as described herein, may selectively inhibit of nNOS, compared to other NOS isoforms, i.e., iNOS and eNOS. As used herein, the term “selective inhibition” can define an affinity to nNOS, determined by the dissociation constant (“K_{d}”) for nNOS, that is at least 10-fold higher, preferably at least 100-fold higher, and most preferably at least 1,000-fold higher than its dissociation constant (“K_{d}”) for eNOS and/or iNOS. In the foregoing, the one or more chemical NOS inhibitors would chemical nNOS inhibitors, as described herein.

In certain embodiments, a NOS inhibitor composition comprising one or more chemical NOS inhibitors, as described herein, may selectively inhibit of iNOS, compared to other NOS isoforms, i.e., nNOS and eNOS. As used herein, the term “selective inhibition” can define an affinity to iNOS, determined by the dissociation constant (“K_{d}”) for iNOS, that is at least 10-fold higher, preferably at least 100-fold higher, and most preferably at least 1,000-fold higher than its dissociation constant (“K_{d}”) for nNOS and/or eNOS. In the foregoing, the one or more chemical NOS inhibitors would chemical iNOS inhibitors, as described herein.

In some embodiments, a NOS inhibitor composition comprising one or more chemical NOS inhibitors, as described herein, may selectively inhibit of eNOS, compared to other NOS isoforms, i.e., nNOS and iNOS. As used herein, the term “selective inhibition” can define an affinity to eNOS, determined by the dissociation constant (“K_{d}”) for eNOS, that is at least 10-fold higher, preferably at least 100-fold higher, and most preferably at least 1,000-fold higher than its dissociation constant (“K_{d}”) for nNOS and/or iNOS. In the foregoing, the one or more chemical NOS inhibitors would chemical eNOS inhibitors, as described herein.

In certain embodiments, a NOS inhibitor composition, as described herein, may be formulated to be administered to a subject via a route selected from the group consisting of buccally, inhalation, intraarterially, intraarticularly, intracardially, intracerebroventricularly, intradermally, intramedullary, intramuscularly, intraperitoneally, intrathecally, intravenously, intraventricularly, locally, nasally, ophthalmically, orally, rectally, sublabially, sublingually, subcutaneously, topically, transdermally, and the like.

To administer a NOS inhibitor composition to a subject via a route, as described herein, the NOS inhibitor composition may be formulated into a suitable dosage form.

For a NOS inhibitor composition formulated to be administered through the oral mucosa, i.e., buccally, sublabially, or sublingually, a suitable dosage form may be, for example, a tablet, a capsule, a pill, a caplet, a dissolvable oral strip, a hydrogel, and the like.

For a NOS inhibitor composition formulated to be administered through injection or infusion, i.e., intraarterially, intraarticularly, intracardially, intracerebroventricularly, intradermally, intramedullary, intramuscularly, intraperitoneally, intrathecally, intravenously, intraventricularly, or subcutaneously, a suitable dosage form may be, for example, (i) a sterile injectable solution, such as, for example, a solution, an emulsion, a suspension, a dispersion, and the like; (ii) a sterile solid, such as, for example, a dispersible powder, a granule, and the like, for the extemporaneous preparation of an aforementioned sterile injectable solution. For a sterile injectable solution, one or more NOS inhibitors, as described herein, can be provided in a non-toxic, parenterally-acceptable aqueous diluent or solvent, such as, for example, 1,3-butanediol, water, Ringer's solution, isotonic sodium chloride solution (saline), ethanol, polyol, glycerol, liquid polyethylene glycols; or alternatively, in a non-toxic, parenterally-acceptable oil diluent or solvent, such as, for example, monoglycerides, diglycerides, oleic acid, vegetable oil, corn oil, and the like; as well as suitable mixtures of the foregoing and any other suitable diluent or solvent not expressly recited that would be readily envisaged to formulate a sterile injectable solution by one skilled in the art. Under ordinary conditions of storage and use, a sterile injectable solution may contain a preservative to prevent the growth of microorganisms, wherein a preservative may be, for example, a paraben, chlorobutanol, phenol, sorbic acid, thimerosal, and the like. In some embodiments, a sterile injectable solution may further comprise isotonic agents, such as, for example, sugars, sodium chloride, and the like, as well as any suitable excipient that would be readily envisaged for inclusion in a sterile injectable solution by one skilled in the art. In certain embodiments, a sterile injectable solution may include excipients that can prolong or delay absorption of one or more NOS inhibitors, such as, for example, aluminum monostearate, gelatin, and the like.

For a NOS inhibitor composition formulated to be administered by inhalation or nasally, a suitable dosage form may be, for example, a spray, an aerosol, and the like.

For a NOS inhibitor composition formulated to be administered by locally, a suitable dosage form may be, for example, an aqueous solution, an oil-based solution, an emulsion, a suspension, a hydrogel, and the like.

For a NOS inhibitor composition formulated to be administered ophthalmically, a suitable dosage form may be, for example, an eye drop, a hydrogel, a contact lens, and the like.

For a NOS inhibitor composition formulated to be administered rectally, a suitable dosage form may be, for example, a suppository, a hydrogel, and the like.

For a NOS inhibitor composition formulated to be administered through the skin, i.e., topically or transdermally, a suitable dosage form may be, for example, a balm, a cream, a foam, a gel, a serum, a salve, a lotion, a paste, a smear, a moisturizer, an oil, an ointment, a jelly, a wax, a patch, a microneedle patch, and the like.

For a NOS inhibitor composition formulated to be administered orally, a suitable dosage form may be a solid dosage form or a liquid dosage form. Solid and liquid dosage forms may, for example, include a powder, a granule, a dispersion, a suspension, an aqueous solution, an oil-based solution, a beverage, a syrup, an elixir, an emulsion, a capsule (soft, hard, gel, or plant-based), a pill, a tablet, a caplet, a sachet, a gum, and the like. In the aforementioned oral formulations, the formulation may comprise one or more excipients. One skilled in the art would recognize the excipients appropriate for the particular oral delivery system contemplated. In addition, various methods of time release and location specific release are contemplated, including immediate release, delayed release, sustained release, controlled release, and targeted delivery.

A NOS inhibitor composition formulated to be administered orally, as described herein, can be prepared according to any method known in the art for the manufacture of pharmaceutically acceptable compositions for oral administration.

In some embodiments, a NOS inhibitor composition formulated to be administered orally, as described herein, may be formulated as a tablet wherein the one or more NOS inhibitors, as described herein, are in admixture with one or more suitable pharmaceutically acceptable excipients. For tablet formulations, suitable pharmaceutically acceptable excipients include, but are not limited to (i) inert diluents, such as, for example, calcium carbonate, sodium carbonate, lactose, calcium phosphate, sodium phosphate, and the like; (ii) granulating and disintegrating agents, such as, for example, corn starch, alginic acid, and the like; (iii) binding agents, such as, for example, starch, gelatin, acacia, and the like; (iv) lubricating agents, such as, for example, magnesium stearate, stearic acid, talc, and the like; (v) sweeteners/flavoring agents; (vi) coloring agents; (vii) coatings; (viii) preservatives, and (ix) any other suitable pharmaceutically acceptable excipients not expressly recited, that would be readily envisaged for inclusion in a tablet by one skilled in the art. In certain embodiments, tablets can be uncoated. In some embodiments, tablets can be coated by known techniques to provide a coating on the tablet that delays disintegration and absorption in the gastrointestinal tract and thereby provide a sustained action over a longer period of time. For example, a time delay material, such as, for example, glyceryl monostearate alone or with a wax, glyceryl distearate alone or with a wax, and the like may be employed.

A NOS inhibitor composition formulated to be administered orally, as described herein, can also be formulated as a hard gelatin-containing or non-gelatinous capsule. In such formulations, one or more NOS inhibitors, as described herein, can be mixed with an inert solid diluent, such as, for example, calcium carbonate, calcium phosphate, kaolin, and the like, as well as any other suitable excipients not expressly recited that would be readily envisaged for inclusion in a hard gelatin-containing or non-gelatinous capsule by one skilled in the art.

A NOS inhibitor composition formulated to be administered orally, as described herein, may be formulated as soft gelatin capsules. In such formulations, one or more NOS inhibitors, as described herein, can be mixed with water or an oil medium, such as, for example, peanut oil, liquid paraffin, olive oil, and the like, and optionally one or more excipients that would be readily envisaged for inclusion in a soft gelatin capsule by one skilled in the art.

A NOS inhibitor composition formulated to be administered orally, as described herein, can also be formulated as an aqueous suspension, wherein one or more NOS inhibitors, as described herein, can be provided in an aqueous solution with one or more excipients. In some embodiments, prior to providing one or more NOS inhibitors, as described herein, in solution, the one or more NOS inhibitors may be prepared as dispersible powders and/or granules. For aqueous solutions, acceptable excipients include, but are not limited to suspending agents, dispersing agents, wetting agents, preservatives, coloring agents, flavoring/sweeting agents, such as, for example, sucrose, saccharin, and the like, and any other suitable excipients not expressly recited that would be readily envisaged for inclusion in an aqueous suspension by one skilled in the art.

A NOS inhibitor composition formulated to be administered orally, as described herein, can also be formulated as an oil suspension, wherein one or more NOS inhibitors, as described herein, can be provided in an oil medium with one or more excipients. For oil suspensions, appropriate oil mediums include, but are not limited to (i) a vegetable oil, such as, for example, arachis oil, olive oil, sesame oil, coconut oil, and the like; (ii) a mineral oil, such as, for example, liquid paraffin, and the like; and (iii) any other suitable oil medium not expressly recited, that would be readily envisaged for use as an oil medium in an oil suspension by one skilled in the art. For oil suspensions, acceptable excipients include, but are not limited to (i) thickening agents, such as, for example, beeswax, hard paraffin, cetyl alcohol, and the like; (ii) sweetening/flavoring agents; (iii) preservatives/antioxidants, such as, for example, ascorbic acid and the like; and (iv) any other suitable excipients not expressly recited that would be readily envisaged for inclusion in an oil suspension by one skilled in the art.

A NOS inhibitor composition formulated to be administered orally, as described herein, may be formulated as a syrup/elixir. For a syrup/elixir, one or more NOS inhibitors, as described herein, can be provided in a solution with one or more excipients. For syrups/elixirs, acceptable excipients include, but are not limited to (i) flavoring/sweetening agents, such as, for example, glycerol, sorbitol, sucrose, and the like; (ii) demulcents; (iii) preservatives; (iv) coloring agents; and (v) any other suitable excipients not expressly recited that would be readily envisaged for inclusion in a syrup/elixir by one skilled in the art.

In some embodiments, a NOS inhibitor composition, as described herein, may be formulated in a controlled release vehicle. As used herein, the term “controlled release vehicle,” refers to a formulation that causes a change in at least one pharmacokinetic property of the active compound(s), i.e., one or more NOS inhibitors, compared to a pharmacokinetic property of the same amount of unformulated active compound(s) administered via the same route. As used herein “pharmacokinetic property,” generally refers to at least one of the following parameters: (i) Cₘₐₓ—the maximum observed concentration of the active compound collected in bodily material, e.g., saliva, urine, plasma, serum, tissue, and the like, of a subject in an in vivo or a clinical study; (ii) Tₘₐₓ—the time it takes to reach Cₘₐₓ; (iii) C_{avg}—the average observed concentration of the active compound collected in bodily material, e.g., saliva, urine, plasma, serum, tissue, and the like, of a subject in an in vivo or a clinical study; (iv) AUC—the total amount exposure to the active compound experienced by a subject in an in vivo or a clinical study; (v) t_{1/2a}—the time required for 50% of a given dose of active compound to be absorbed into the systemic circulation; (vi) t_{1/2b}—the time required for the concentration of the active compound to reach half of its original value; (vii) C*ₘₐₓ—the maximum concentration of the active compound observed in an in vitro release experiment; (viii) T*ₘₐₓ—the time it takes to reach C*ₘₐₓ; (ix) C*_{avg}—the average observed concentration of the active compound observed in an in vitro release experiment; (x) AUC*—the total amount of the active compound released from the formulation in and in vitro release experiment; (xi) t*1/2—the time required for 50% of the active compound to be released from the formulation in an in vitro release experiment; (xii) t^{∞}—the time at which the active compound is no longer released from the formulation in an in vitro release experiment; as well as any other suitable parameter not expressly listed that would be readily envisaged as “controlled release” by one skilled in the art.

The following terms may be considered to be substantially equivalent to “controlled release” for the purposes of the present disclosure: continuous release, controlled release, delayed release, depot, gradual release, long term release, programmed release, prolonged release, programmed release, proportionate release, protracted release, repository, retard, slow release, spaced release, sustained release, time coat, time release, delayed action, extended action, layered time action, long acting, prolonged action, sustained action medications and extended release, release in terms of pH level in the gut and intestine, breakdown of the molecule and based on the absorption and bioavailability.

A NOS inhibitor composition formulated in a controlled release vehicle, as described herein, can be formulated to be administered via the routes disclosed herein. In such formulations, controlled release may occur from the excipients provided in the formulations. Exemplary excipients that can achieve controlled release include, but are not limited to (i) biodegradable or bioerodable polymers, such as, for example, polylactic acid, polyglycolic acid, regenerated collagen, and the like; (ii) macromolecules, such as, for example, polyesters, polyamino acids, polyvinylpyrrolidone, ethylenevinyl acetate, methylcellulose, carboxymethylcellulose, protamine sulfate, and the like.

A NOS inhibitor composition formulated in a controlled release vehicle, as described herein, may be formulated as a hydrogel, wherein the NOS inhibitor composition can be provided within the matrix of a carrier. In some embodiments, a carrier, as described herein, can be prepared by copolymerization of hydrophilic mono-olefinic monomers, such as, for example, ethylene glycol methacrylate, and the like. In certain embodiments, a carrier, as described herein, may comprise a silicon rubber. In some embodiments, a carrier, as described herein, may comprise ethylene-vinyl alcohol. In certain embodiments, a carrier, as described herein, can be porous, non-porous, solid, semi-solid, permeable, or impermeable, which may be used to impart distinct release kinetics of the NOS inhibitor composition. In some embodiments, a carrier may be encapsulated by a rate controlling membrane, such as, for example, an ethylene-vinyl acetate copolymer membrane, a butylene terephthalate/polytetramethylene ether terephthalate membrane, and the like, which can be used to modulate the release of the NOS inhibitor composition.

A NOS inhibitor composition formulated in a controlled release vehicle, as described herein, may be formulated as an oral formulation. In some embodiments, a NOS inhibitor composition, as described herein, can be incorporated into a soluble or erodible controlled release matrix, such as a pill or a lozenge. In certain embodiments, a NOS inhibitor composition, as described herein, can be formulated into a controlled release gel or paste.

In some embodiments, suitable dosage forms, as described herein, may comprise one or more excipients. The excipients are not particularly limited and may include, but are not limited to: (i) acidifying agents, such as, for example, acetic acid, glacial acetic acid, citric acid, fumaric acid, hydrochloric acid, diluted hydrochloric acid, malic acid, nitric acid, phosphoric acid, diluted phosphoric acid, sulfuric acid, tartaric acid, and the like; (ii) alkalizing agents, such as, for example, ammonia solution, ammonium carbonate, diethanolamine, diisopropanolamine, potassium hydroxide, sodium bicarbonate, sodium borate, sodium carbonate, sodium hydroxide, trolamine, and the like; (iii) antifoaming agents, such as, for example, dimethicone, simethicone, and the like; (iv) antimicrobial preservatives, such as, for example, benzalkonium chloride, benzalkonium chloride solution, benzethonium chloride, benzoic acid, benzyl alcohol, butylparaben, cetylpyridinium chloride, chlorobutanol, chlorocresol, cresol, dehydroacetic acid, ethylparaben, methylparaben, methylparaben sodium, phenol, phenylethyl alcohol, phenylmercuric acetate, phenylmercuric nitrate, potassium benzoate, potassium sorbate, propylparaben, propylparaben sodium, sodium benzoate, sodium dehydroacetate, sodium propionate, sorbic acid, thimerosal, thymol, and the like; (v) antioxidants, such as, for example, ascorbic acid, ascorbyl palmitate, butylated hydroxyanisole, butylated hydroxytoluene, hypophosphorous acid, monothioglycerol, propyl gallate, sodium formaldehyde sulfoxylate, sodium metabisulfite, sodium thiosulfate, sulfur dioxide, tocopherol, tocopherols excipient, and the like; (vi) buffering agents, such as, for example, acetic acid, ammonium carbonate, ammonium phosphate, boric acid, citric acid, lactic acid, phosphoric acid, potassium citrate, potassium metaphosphate, potassium phosphate monobasic, sodium acetate, sodium citrate, sodium lactate solution, dibasic sodium phosphate, monobasic sodium phosphate, and the like; (vii) chelating agents, such as, for example, edetate disodium, ethylenediaminetetraacetic acid and salts, edetic acid, and the like; (viii) coating agents, such as, for example, sodium carboxymethylcellulose, cellulose acetate, cellulose acetate phthalate, ethylcellulose, gelatin, pharmaceutical glaze, hydroxypropyl cellulose, hydroxypropyl methylcellulose, hydroxypropyl methylcellulose phthalate, methacrylic acid copolymer, methylcellulose, polyvinyl acetate phthalate, shellac, sucrose, titanium dioxide, carnauba wax, microcrystalline wax, zein, and the like; (ix) colorants, such as, for example, caramel, red, yellow, black or blends, ferric oxide, and the like; (x) complexing agents, such as, for example, ethylenediaminetetraacetic acid and salts (EDTA), edetic acid, gentisic acid ethanolamide, oxyquinoline sulfate, and the like; (xi) desiccants, such as, for example, calcium chloride, calcium sulfate, silicon dioxide, and the like; (xii) emulsifying and/or solubilizing agents, such as, for example, acacia, cholesterol, diethanolamine (adjunct), glyceryl monostearate, lanolin alcohols, mono- and di-glycerides, monoethanolamine (adjunct), lecithin, oleic acid (adjunct), oleyl alcohol (stabilizer), poloxamer, polyoxyethylene 50 stearate, polyoxyl 35 castor oil, polyoxyl 40 hydrogenated castor oil, polyoxyl 10 oleyl ether, polyoxyl 20 cetostearyl ether, polyoxyl 40 stearate, polysorbate 20, polysorbate 40, polysorbate 60, polysorbate 80, diacetate, monostearate, sodium lauryl sulfate, sodium stearate, sorbitan monolaurate, sorbitan monooleate, sorbitan monopalmitate, sorbitan monostearate, stearic acid, trolamine, emulsifying wax, and the like; (xiii) filtering aids, such as, for example, powdered cellulose, purified siliceous earth, and the like; (xiv) flavors and perfumes, such as, for example, anethole, benzaldehyde, ethyl vanillin, menthol, methyl salicylate, monosodium glutamate, orange flower oil, peppermint, peppermint oil, peppermint spirit, rose oil, stronger rose water, thymol, tolu balsam tincture, vanilla, vanilla tincture, vanillin, and the like; (xv) humectants, such as, for example, glycerol, hexylene glycol, sorbitol, and the like; (xvi) plasticizers, such as, for example, castor oil, diacetylated monoglycerides, diethyl phthalate, glycerol, mono- and di-acetylated monoglycerides, propylene glycol, triacetin, triethyl citrate, and the like; (xvii) polymers, such as, for example, cellulose acetate, alkyl celluloses, hydroxyalkyl, acrylic polymers and copolymers, and the like; (xviii) solvents, such as, for example, acetone, alcohol, diluted alcohol, amylene hydrate, benzyl benzoate, butyl alcohol, carbon tetrachloride, chloroform, corn oil, cottonseed oil, dimethyl sulfoxide, ethyl acetate, glycerol, hexylene glycol, isopropyl alcohol, methyl alcohol, methylene chloride, methyl isobutyl ketone, mineral oil, peanut oil, propylene carbonate, sesame oil, water for injection, sterile water for injection, sterile water for irrigation, purified water, and the like; (xix) sorbents, such as, for example, powdered cellulose, charcoal, purified siliceous earth, and the like; (xx) carbon dioxide sorbents, such as, for example, barium hydroxide lime, soda lime, and the like; (xxi) stiffening agents, such as, for example, hydrogenated castor oil, cetostearyl alcohol, cetyl alcohol, cetyl esters wax, hard fat, paraffin, polyethylene excipient, stearyl alcohol, emulsifying wax, white wax, yellow wax, and the like; (xxii) suspending and/or thickening agents, such as, for example, acacia, agar, alginic acid, aluminum monostearate, bentonite, purified bentonite, magma bentonite, carbomer, carboxymethylcellulose calcium, carboxymethylcellulose sodium, carboxymethylcellulose sodium 12, carrageenan, microcrystalline and carboxymethylcellulose sodium cellulose, dextrin, gelatin (Bloom strength 50-100), guar gum, hydroxyethyl cellulose, hydroxypropyl cellulose, hydroxypropyl methylcellulose, magnesium aluminum silicate, methylcellulose, pectin, polyethylene oxide, polyvinyl alcohol, povidone, alginate, silicon dioxide, colloidal silicon dioxide, sodium alginate, tragacanth, xanthan gum, and the like; (xxiii) sweetening agents, such as, for example, aspartame, dextrates, dextrose, excipient dextrose, fructose, mannitol, saccharin, calcium saccharin, sodium saccharin, sorbitol, solution sorbitol, sucrose, compressible sugar, confectioner's sugar, syrup, and the like; (xxiv) surfactants, such as, for example, simethicone, and the like; (xxv) tablet binders, such as, for example, acacia, alginic acid, sodium carboxymethylcellulose, microcrystalline cellulose, dextrin, ethylcellulose, gelatin, liquid glucose, guar gum, hydroxypropyl methylcellulose, methylcellulose, polyethylene oxide, povidone, pregelatinized starch, syrup, and the like; (xxvi) tablet and/or capsule diluents, such as, for example, calcium carbonate, dibasic calcium phosphate, tribasic calcium phosphate, calcium sulfate, microcrystalline cellulose, powdered cellulose, dextrates, dextrin, dextrose excipient, fructose, kaolin, lactose, mannitol, sorbitol, starch, pregelatinized starch, sucrose, compressible sugar, confectioner's sugar, and the like; (xxvii) tablet disintegrants, such as, for example, alginic acid, microcrystalline cellulose, croscarmellose sodium, crospovidone, polacrilin potassium, sodium starch glycolate, starch, pregelatinized starch, and the like; (xxviii) tablet and/or capsule lubricants, such as, for example, calcium stearate, glyceryl behenate, magnesium stearate, light mineral oil, sodium stearyl fumarate, stearic acid, purified stearic acid, talc, hydrogenated vegetable oil, zinc stearate, and the like; (xxix) tonicity agent, such as, for example, dextrose, glycerol, mannitol, potassium chloride, sodium chloride, and the like; (xxx) vehicle: flavored and/or sweetened, such as, for example, aromatic elixir, compound benzaldehyde elixir, iso-alcoholic elixir, peppermint water, sorbitol solution, syrup, tolu balsam syrup, and the like; (xxxi) vehicle: oleaginous, such as, for example, almond oil, corn oil, cottonseed oil, ethyl oleate, isopropyl myristate, isopropyl palmitate, mineral oil, light mineral oil, myristyl alcohol, octyl dodecanol, olive oil, peanut oil, persic oil, sesame oil, soybean oil, squalane, and the like; (xxxii) vehicle: solid carrier, such as, for example, sugar spheres, and the like; (xxxiii) vehicle: sterile, such as, for example, bacteriostatic water for injection, bacteriostatic sodium chloride injection, and the like; (xxxiv) water repelling agents, such as, for example, cyclomethicone, dimethicone, simethicone, and the like; and/or (xxxv) solubilizing agent, such as, for example, benzalkonium chloride, benzethonium chloride, cetylpyridinium chloride, docusate sodium, nonoxynol 9, nonoxynol 10, octoxynol 9, poloxamer, polyoxyl 35 castor oil, polyoxyl 40, hydrogenated castor oil, polyoxyl 50 stearate, polyoxyl 10 oleyl ether, polyoxyl 20 cetostearyl ether, polyoxyl 40 stearate, polysorbate 20, polysorbate 40, polysorbate 60, polysorbate 80, sodium lauryl sulfate, sorbitan monolaurate, sorbitan monooleate, sorbitan monopalmitate, sorbitan monostearate, tyloxapol, and the like. This list is not meant to be exclusive, but instead merely representative of the classes of excipients and the particular excipients that may be used in the formulations described herein.

In some embodiments, a NOS inhibitor composition, as described herein, may be formulated to deliver the NOS inhibitor composition to the particular site of the malignancy via a targeting strategy. As used herein, the term “targeting strategy” generally refers to formulating the NOS inhibitor composition in a manner that improves the accumulation of the NOS inhibitor composition at the site of the malignancy when the NOS inhibitor composition is systemically administered, i.e., intravenously, intraperitoneally, subcutaneously, etc., compared to the expected accumulation of the NOS inhibitor composition formulated absent a targeting strategy. In some embodiments, a targeting strategy may be achieved by formulating the NOS inhibitor composition in a delivery system of a sufficient size to leverage the enhanced permeability and retention (“EPR”) effect. The EPR effect refers to the phenomenon wherein drug delivery carriers below a certain size threshold will accumulate preferentially at a cancer/tumor site compared to normal tissue owing to the leaky vasculature of cancers and tumors. In certain embodiments, a targeting strategy may be achieved by using a NOS inhibitor composition that has a targeting affinity for the particular cancer disclosed. By way of example, such a targeting strategy may be achieved by administering a compound that has a strong affinity for a particular NOS isoform, such that a disproportionate amount of the compound accumulates in tissue where the particular NOS isoform is overexpressed. In some embodiments, a targeting strategy may be achieved by formulating the NOS inhibitor composition to comprise a targeting moiety. By way of example, a targeting moiety, as described herein, may be (i) formulated on the surface of a formulation comprising the NOS inhibitor composition, e.g., on the surface of a liposome containing the NOS inhibitor composition within the liposome, (ii) conjugated directly to NOS inhibitor composition, or (iii) conjugated to the NOS inhibitor composition via a linker such that the resulting construct can be cleaved in vivo to release NOS inhibitor composition. By way of example, a targeting moiety may define one or more of the following: an antibody, a protein, a glycoprotein, a macromolecule, biotin, streptavidin, folic acid, transferrin, hyaluronic acid, an oligosaccharides, a polysaccharides, an aptamer, an oligopeptide, a peptide, a nucleic acid construct, a polymer, and the like.

In certain embodiments, a NOS inhibitor composition, as described herein, may be administered as a suitable dosage form, as described herein, to treat, ameliorate, and/or reduce the severity of glioblastoma or one or more symptoms thereof in a subject (“glioblastoma subject”).

In some embodiments, a NOS inhibitor composition comprising 7-NI and/or a salt thereof may be administered as a suitable dosage form, as described herein, to treat, ameliorate, and/or reduce the severity of glioblastoma or one or more symptoms thereof in a glioblastoma subject. In certain embodiments, an nNOS inhibitor composition comprising 7-NI and/or a salt thereof may be administered to a glioblastoma subject to treat, ameliorate, and/or reduce the severity of glioblastoma or one or more symptoms thereof in the glioblastoma subject: (i) via a route selected from the group consisting of buccally, inhalation, intraarterially, intraarticularly, intracardially, intracerebroventricularly, intradermally, intramedullary, intramuscularly, intraperitoneally, intrathecally, intravenously, intraventricularly, nasally, ophthalmically, orally, rectally, sublabially, sublingually, subcutaneously, topically, and transdermally; and/or (ii) with a frequency selected from the group consisting of four times daily, three times daily, twice daily, once daily, once every two days, once every three days, once every four days, once every five days, once every six days, once a week, once every eight days, once every nine days, once every ten days, once every two weeks, once every three weeks, and once a month; and/or (iii) at a dosage (based on subject weight) selected from the group consisting of 0.1 μg/kg, 0.5 μg/kg, 1 μg/kg, 1.5 μg/kg, 2.0 μg/kg, 3.0 μg/kg, 3.5 μg/kg, 4.0 μg/kg, 4.5 μg/kg, 5.0 μg/kg, 10 μg/kg, 15 μg/kg, 20 μg/kg, 25 μg/kg, 30 μg/kg, 35 μg/kg, 40 μg/kg, 45 μg/kg, 50 μg/kg, 55 μg/kg, 60 μg/kg, 65 μg/kg, 70 μg/kg, 75 μg/kg, 80 μg/kg, 85 μg/kg, 90 μg/kg, 95 μg/kg, 100 μg/kg, 150 μg/kg, 200 μg/kg, 250 μg/kg, 300 μg/kg, 350 μg/kg, 400 μg/kg, 450 μg/kg, 500 μg/kg, 550 μg/kg, 600 μg/kg, 650 μg/kg, 700 μg/kg, 750 μg/kg, 800 μg/kg, 850 μg/kg, 900 μg/kg, 1 mg/kg, 1.5 mg/kg, 2.0 mg/kg, 2.5 mg/kg, 3 mg/kg, 3.5 mg/kg, 4.0 mg/kg, 4.5 mg/kg, 5 mg/kg, 5.5 mg/kg, 6 mg/kg, 6.5 mg/kg, 7 mg/kg, 7.5 mg/kg, 8 mg/kg, 8.5 mg/kg, 9 mg/kg, 9.5 mg/kg, 10 mg/kg, 10.5 mg/kg, 11 mg/kg, 11.5 mg/kg, 12 mg/kg, 12.5 mg/kg, 13 mg/kg, 13.5 mg/kg, 14 mg/kg, 14.5 mg/kg, 15 mg/kg, 16 mg/kg, 17 mg/kg, 18 mg/kg, 19 mg/kg, 20 mg/kg, 21 mg/kg, 22 mg/kg, 22 mg/kg, 23 mg/kg, 24 mg/kg, 25 mg/kg, 30 mg/kg, 35 mg/kg, 40 mg/kg, 45 mg/kg, 50 mg/kg, 55 mg/kg, 60 mg/kg, 65 mg/kg, 70 mg/kg, 75 mg/kg, 80 mg/kg, 85 mg/kg, 90 mg/kg, 95 mg/kg, 100 mg/kg, 105 mg/kg, 110 mg/kg, 115 mg/kg, 120 mg/kg, 125 mg/kg, 130 mg/kg, 135 mg/kg, 140 mg/kg, 145 mg/kg, 150 mg/kg, 155 mg/kg, 160 mg/kg, 165 mg/kg, 170 mg/kg, 175 mg/kg, 180 mg/kg, 185 mg/kg, 190 mg/kg, 195 mg/kg, 200 mg/kg, and any fraction or integer in between any two of the preceding amounts.

In certain embodiments, a NOS inhibitor composition comprising 3-Br-7-NI and/or a salt thereof may be administered as a suitable dosage form, as described herein, to treat, ameliorate, and/or reduce the severity of glioblastoma or one or more symptoms thereof in a glioblastoma subject. In some embodiments, an nNOS inhibitor composition comprising 3-Br-7-NI and/or a salt thereof may be administered to a glioblastoma subject to treat, ameliorate, and/or reduce the severity of glioblastoma or one or more symptoms thereof in the glioblastoma subject: (i) via a route selected from the group consisting of buccally, inhalation, intraarterially, intraarticularly, intracardially, intracerebroventricularly, intradermally, intramedullary, intramuscularly, intraperitoneally, intrathecally, intravenously, intraventricularly, nasally, ophthalmically, orally, rectally, sublabially, sublingually, subcutaneously, topically, and transdermally; and/or (ii) with a frequency selected from the group consisting of four times daily, three times daily, twice daily, once daily, once every two days, once every three days, once every four days, once every five days, once every six days, once a week, once every eight days, once every nine days, once every ten days, once every two weeks, once every three weeks, and once a month; and/or (iii) at a dosage (based on subject weight) selected from the group consisting of 0.1 μg/kg, 0.5 μg/kg, 1 μg/kg, 1.5 μg/kg, 2.0 μg/kg, 2.5 μg/kg, 3.0 μg/kg, 3.5 μg/kg, 4.0 μg/kg, 4.5 μg/kg, 5.0 μg/kg, 10 μg/kg, 15 μg/kg, 20 μg/kg, 25 μg/kg, 30 μg/kg, 35 μg/kg, 40 μg/kg, 45 μg/kg, 50 μg/kg, 55 μg/kg, 60 μg/kg, 65 μg/kg, 70 μg/kg, 80 μg/kg, 85 μg/kg, 90 μg/kg, 100 μg/kg, 150 μg/kg, 200 μg/kg, 250 μg/kg, 300 μg/kg, 350 μg/kg, 400 μg/kg, 450 μg/kg, 500 μg/kg, 550 μg/kg, 600 μg/kg, 650 μg/kg, 700 μg/kg, 750 μg/kg, 800 μg/kg, 850 μg/kg, 900 μg/kg, 1 mg/kg, 1.5 mg/kg, 2.0 mg/kg, 2.5 mg/kg, 3 mg/kg, 3.5 mg/kg, 4.0 mg/kg, 4.5 mg/kg, 5 mg/kg, 5.5 mg/kg, 6 mg/kg, 6.5 mg/kg, 7 mg/kg, 7.5 mg/kg, 8 mg/kg, 8.5 mg/kg, 9 mg/kg, 9.5 mg/kg, 10 mg/kg, 10.5 mg/kg, 11 mg/kg, 11.5 mg/kg, 12 mg/kg, 12.5 mg/kg, 13 mg/kg, 13.5 mg/kg, 14 mg/kg, 14.5 mg/kg, 15 mg/kg, 16 mg/kg, 17 mg/kg, 18 mg/kg, 19 mg/kg, 20 mg/kg, 21 mg/kg, 22 mg/kg, 23 mg/kg, 24 mg/kg, 25 mg/kg, 30 mg/kg, 35 mg/kg, 40 mg/kg, 45 mg/kg, 50 mg/kg, 55 mg/kg, 60 mg/kg, 65 mg/kg, 70 mg/kg, 75 mg/kg, 80 mg/kg, 85 mg/kg, 90 mg/kg, 95 mg/kg, 100 mg/kg, 105 mg/kg, 110 mg/kg, 115 mg/kg, 120 mg/kg, 125 mg/kg, 130 mg/kg, 135 mg/kg, 140 mg/kg, 145 mg/kg, 150 mg/kg, 155 mg/kg, 160 mg/kg, 165 mg/kg, 170 mg/kg, 175 mg/kg, 180 mg/kg, 185 mg/kg, 190 mg/kg, 195 mg/kg, 200 mg/kg, and any fraction or integer in between any two of the preceeding amounts.

In some embodiments, a NOS inhibitor composition comprising L-NPA and/or a salt thereof, such as, for example, L-NPA hydrochloride, may be administered as a suitable dosage form, as described herein, to treat, ameliorate, and/or reduce the severity of glioblastoma or one or more symptoms thereof in a glioblastoma subject. In certain embodiments, an nNOS inhibitor composition comprising L-NPA and/or a salt thereof, such as, for example, L-NPA hydrochloride may be administered to a glioblastoma subject to treat, ameliorate, and/or reduce the severity of glioblastoma or one or more symptoms thereof in the glioblastoma subject: (i) via a route selected from the group consisting of buccally, inhalation, intraarterially, intraarticularly, intracardially, intracerebroventricularly, intradermally, intramedullary, intramuscularly, intraperitoneally, intrathecally, intravenously, intraventricularly, nasally, ophthalmically, orally, rectally, sublabially, sublingually, subcutaneously, topically, and transdermally; and/or (ii) with a frequency selected from the group consisting of four times daily, three times daily, twice daily, once daily, once every two days, once every three days, once every four days, once every five days, once every six days, once a week, once every eight days, once every nine days, once every ten days, once every two weeks, once every three weeks, and once a month; and/or (iii) at a dosage (based on subject weight) selected from the group consisting of 0.1 μg/kg, 0.5 μg/kg, 1 μg/kg, 1.5 g/kg, 2.0 μg/kg, 2.5 μg/kg, 3.0 μg/kg, 3.5 μg/kg, 4.0 μg/kg, 4.5 μg/kg, 5.0 μg/kg, 10 μg/kg, 15 g/kg, 20 μg/kg, 25 μg/kg, 30 μg/kg, 35 μg/kg, 40 μg/kg, 45 μg/kg, 50 μg/kg, 55 μg/kg, 60 μg/kg, 65 μg/kg, 70 μg/kg, 75 μg/kg, 80 μg/kg, 85 μg/kg, 90 μg/kg, 95 μg/kg, 100 μg/kg, 150 μg/kg, 200 g/kg, 250 μg/kg, 300 μg/kg, 350 μg/kg, 400 μg/kg, 450 μg/kg, 500 μg/kg, 550 μg/kg, 600 μg/kg, 650 μg/kg, 700 μg/kg, 750 μg/kg, 800 μg/kg, 850 μg/kg, 900 μg/kg, 1 mg/kg, 1.5 mg/kg, 2.0 mg/kg, 2.5 mg/kg, 3 mg/kg, 3.5 mg/kg, 4.0 mg/kg, 4.5 mg/kg, 5 mg/kg, 5.5 mg/kg, 6 mg/kg, 6.5 mg/kg, 7 mg/kg, 7.5 mg/kg, 8 mg/kg, 8.5 mg/kg, 9 mg/kg, 9.5 mg/kg, 10 mg/kg 10.5 mg/kg, 11 mg/kg, 11.5 mg/kg, 12 mg/kg, 12.5 mg/kg, 13 mg/kg, 13.5 mg/kg, 14 mg/kg, 14.5 mg/kg, 15 mg/kg, 16 mg/kg, 17 mg/kg, 18 mg/kg, 19 mg/kg, 20 mg/kg, 21 mg/kg, 22 mg/kg, 23 mg/kg, 24 mg/kg, 25 mg/kg, 30 mg/kg, 35 mg/kg, 40 mg/kg, 45 mg/kg, 50 mg/kg, 55 mg/kg, 60 mg/kg, 65 mg/kg, 70 mg/kg, 75 mg/kg, 80 mg/kg, 85 mg/kg, 90 mg/kg, 95 mg/kg, 100 mg/kg, 105 mg/kg, 110 mg/kg, 115 mg/kg, 120 mg/kg, 125 mg/kg, 130 mg/kg, 135 mg/kg, 140 mg/kg, 145 mg/kg, 150 mg/kg, 155 mg/kg, 160 mg/kg, 165 mg/kg, 170 mg/kg, 175 mg/kg, 180 mg/kg, 185 mg/kg, 190 mg/kg, 195 mg/kg, 200 mg/kg, and any fraction or integer in between any two of the preceding amounts.

In certain embodiments, a NOS inhibitor composition comprising L-NIO and/or a salt thereof, such as, for example, L-NIO dihydrochloride, may be administered as a suitable dosage form, as described herein, to treat, ameliorate, and/or reduce the severity of glioblastoma or one or more symptoms thereof in a glioblastoma subject. In some embodiments, an nNOS inhibitor composition comprising L-NIO and/or a salt thereof, such as, for example, L-NIO dihydrochloride, may be administered to a glioblastoma subject to treat, ameliorate, and/or reduce the severity of glioblastoma or one or more symptoms thereof in the glioblastoma subject: (i) via a route selected from the group consisting of buccally, inhalation, intraarterially, intraarticularly, intracardially, intracerebroventricularly, intradermally, intramedullary, intramuscularly, intraperitoneally, intrathecally, intravenously, intraventricularly, nasally, ophthalmically, orally, rectally, sublabially, sublingually, subcutaneously, topically, and transdermally; and/or (ii) with a frequency selected from the group consisting of four times daily, three times daily, twice daily, once daily, once every two days, once every three days, once every four days, once every five days, once every six days, once a week, once every eight days, once every nine days, once every ten days, once every two weeks, once every three weeks, and once a month; and/or (iii) at a dosage (based on subject weight) selected from the group consisting of 0.1 μg/kg, 0.5 μg/kg, 1 μg/kg, 1.5 μg/kg, 2.0 μg/kg, 2.5 μg/kg, 3.0 μg/kg, 3.5 μg/kg, 4.0 μg/kg, 4.5 μg/kg, 5.0 μg/kg, 10 μg/kg, 15 μg/kg, 20 μg/kg, 25 μg/kg, 30 μg/kg, 35 μg/kg, 40 μg/kg, 45 μg/kg, 50 μg/kg, 55 μg/kg, 60 μg/kg, 65 μg/kg, 70 μg/kg, 75 μg/kg, 80 μg/kg, 85 μg/kg, 90 μg/kg, 95 μg/kg, 100 μg/kg, 150 μg/kg, 200 μg/kg, 250 μg/kg, 300 μg/kg, 350 μg/kg, 400 μg/kg, 450 μg/kg, 500 μg/kg, 550 μg/kg, 600 μg/kg, 650 μg/kg, 700 μg/kg, 750 μg/kg, 800 μg/kg, 850 μg/kg, 900 μg/kg, 1 mg/kg, 1.5 mg/kg, 2.0 mg/kg, 2.5 mg/kg, 3 mg/kg, 3.5 mg/kg, 4.0 mg/kg, 4.5 mg/kg, 5 mg/kg, 5.5 mg/kg, 6 mg/kg, 6.5 mg/kg, 7 mg/kg, 7.5 mg/kg, 8 mg/kg, 8.5 mg/kg, 9 mg/kg, 9.5 mg/kg, 10 mg/kg 10.5 mg/kg, 11 mg/kg, 11.5 mg/kg, 12 mg/kg, 12.5 mg/kg, 13 mg/kg, 13.5 mg/kg, 14 mg/kg, 14.5 mg/kg, 15 mg/kg, 16 mg/kg, 17 mg/kg, 18 mg/kg, 19 mg/kg, 20 mg/kg, 21 mg/kg, 22 mg/kg, 23 mg/kg, 24 mg/kg, 25 mg/kg, 30 mg/kg, 35 mg/kg, 40 mg/kg, 45 mg/kg, 50 mg/kg, 55 mg/kg, 60 mg/kg, 65 mg/kg, 70 mg/kg, 75 mg/kg, 80 mg/kg, 85 mg/kg, 90 mg/kg, 95 mg/kg, 100 mg/kg, 105 mg/kg, 110 mg/kg, 115 mg/kg, 120 mg/kg, 125 mg/kg, 130 mg/kg, 135 mg/kg, 140 mg/kg, 145 mg/kg, 150 mg/kg, 155 mg/kg, 160 mg/kg, 165 mg/kg, 170 mg/kg, 175 mg/kg, 180 mg/kg, 185 mg/kg, 190 mg/kg, 195 mg/kg, 200 mg/kg, and any fraction or integer in between any two of the preceding amounts.

In some embodiments, a NOS inhibitor composition comprising ARL-17477 and/or a salt thereof, such as, for example, ARL-17477 dihydrochloride, may be administered as a suitable dosage form, as described herein, to treat, ameliorate, and/or reduce the severity of glioblastoma or one or more symptoms thereof in a glioblastoma subject. In certain embodiments, an nNOS inhibitor composition comprising ARL-17477 and/or a salt thereof, such as, for example, ARL-17477 dihydrochloride, may be administered to a glioblastoma subject to treat, ameliorate, and/or reduce the severity of glioblastoma or one or more symptoms thereof in the glioblastoma subject: (i) via a route selected from the group consisting of buccally, inhalation, intraarterially, intraarticularly, intracardially, intracerebroventricularly, intradermally, intramedullary, intramuscularly, intraperitoneally, intrathecally, intravenously, intraventricularly, nasally, ophthalmically, orally, rectally, sublabially, sublingually, subcutaneously, topically, and transdermally; and/or (ii) with a frequency selected from the group consisting of four times daily, three times daily, twice daily, once daily, once every two days, once every three days, once every four days, once every five days, once every six days, once a week, once every eight days, once every nine days, once every ten days, once every two weeks, once every three weeks, and once a month; and/or (iii) at a dosage (based on subject weight) selected from the group consisting of 0.1 μg/kg, 0.5 μg/kg, 1 μg/kg, 1.5 μg/kg, 2.0 μg/kg, 2.5 μg/kg, 3.0 μg/kg, 3.5 μg/kg, 4.0 μg/kg, 4.5 μg/kg, 5.0 μg/kg, 10 μg/kg, 15 μg/kg, 20 μg/kg, 25 μg/kg, 30 μg/kg, 35 μg/kg, 40 μg/kg, 45 μg/kg, 50 μg/kg, 55 μg/kg, 60 μg/kg, 65 μg/kg, 70 μg/kg, 75 μg/kg, 80 μg/kg, 85 μg/kg, 90 μg/kg, 95 μg/kg, 100 μg/kg, 150 μg/kg, 200 μg/kg, 250 μg/kg, 300 μg/kg, 350 μg/kg, 400 μg/kg, 450 μg/kg, 500 μg/kg, 550 μg/kg, 600 μg/kg, 650 μg/kg, 700 μg/kg, 750 μg/kg, 800 μg/kg, 850 μg/kg, 900 μg/kg, 1 mg/kg, 1.5 mg/kg, 2.0 mg/kg, 2.5 mg/kg, 3 mg/kg, 3.5 mg/kg, 4.0 mg/kg, 4.5 mg/kg, 5 mg/kg, 5.5 mg/kg, 6 mg/kg, 6.5 mg/kg, 7 mg/kg, 7.5 mg/kg, 8 mg/kg, 8.5 mg/kg, 9 mg/kg, 9.5 mg/kg, 10 mg/kg 10.5 mg/kg, 11 mg/kg, 11.5 mg/kg, 12 mg/kg, 12.5 mg/kg, 13 mg/kg, 13.5 mg/kg, 14 mg/kg, 14.5 mg/kg, 15 mg/kg, 16 mg/kg, 17 mg/kg, 18 mg/kg, 19 mg/kg, 20 mg/kg, 21 mg/kg, 22 mg/kg, 23 mg/kg, 24 mg/kg, 25 mg/kg, 30 mg/kg, 35 mg/kg, 40 mg/kg, 45 mg/kg, 50 mg/kg, 55 mg/kg, 60 mg/kg, 65 mg/kg, 70 mg/kg, 75 mg/kg, 80 mg/kg, 85 mg/kg, 90 mg/kg, 95 mg/kg, 100 mg/kg, 105 mg/kg, 110 mg/kg, 115 mg/kg, 120 mg/kg, 125 mg/kg, 130 mg/kg, 135 mg/kg, 140 mg/kg, 145 mg/kg, 150 mg/kg, 155 mg/kg, 160 mg/kg, 165 mg/kg, 170 mg/kg, 175 mg/kg, 180 mg/kg, 185 mg/kg, 190 mg/kg, 195 mg/kg, 200 mg/kg, and any fraction or integer in between any two of the preceding amounts.

In certain embodiments, a NOS inhibitor composition comprising L-NNA and/or a salt thereof may be administered as a suitable dosage form, as described herein, to treat, ameliorate, and/or reduce the severity of glioblastoma or one or more symptoms thereof in a glioblastoma subject. In some embodiments, an nNOS inhibitor composition comprising L-NNA and/or a salt thereof may be administered to a glioblastoma subject to treat, ameliorate, and/or reduce the severity of glioblastoma or one or more symptoms thereof in the glioblastoma subject: (i) via a route selected from the group consisting of buccally, inhalation, intraarterially, intraarticularly, intracardially, intracerebroventricularly, intradermally, intramedullary, intramuscularly, intraperitoneally, intrathecally, intravenously, intraventricularly, nasally, ophthalmically, orally, rectally, sublabially, sublingually, subcutaneously, topically, and transdermally; and/or (ii) with a frequency selected from the group consisting of four times daily, three times daily, twice daily, once daily, once every two days, once every three days, once every four days, once every five days, once every six days, once a week, once every eight days, once every nine days, once every ten days, once every two weeks, once every three weeks, and once a month; and/or (iii) at a dosage (based on subject weight) selected from the group consisting of 0.1 μg/kg, 0.5 μg/kg, 1 μg/kg, 1.5 μg/kg, 2.0 μg/kg, 2.5 μg/kg, 3.0 μg/kg, 3.5 μg/kg, 4.0 μg/kg, 4.5 μg/kg, 5.0 μg/kg, 10 μg/kg, 15 μg/kg, 20 μg/kg, 25 μg/kg, 30 μg/kg, 35 μg/kg, 40 μg/kg, 45 μg/kg, 50 μg/kg, 55 μg/kg, 60 μg/kg, 65 μg/kg, 70 μg/kg, 75 μg/kg, 80 μg/kg, 85 μg/kg, 90 μg/kg, 95 μg/kg, 100 μg/kg, 150 μg/kg, 200 μg/kg, 250 μg/kg, 300 μg/kg, 350 μg/kg, 400 μg/kg, 450 μg/kg, 500 μg/kg, 550 μg/kg, 600 μg/kg, 650 μg/kg, 700 μg/kg, 750 μg/kg, 800 μg/kg, 850 μg/kg, 900 μg/kg, 1 mg/kg, 1.5 mg/kg, 2.0 mg/kg, 2.5 mg/kg, 3 mg/kg, 3.5 mg/kg, 4.0 mg/kg, 4.5 mg/kg, 5 mg/kg, 5.5 mg/kg, 6 mg/kg, 6.5 mg/kg, 7 mg/kg, 7.5 mg/kg, 8 mg/kg, 8.5 mg/kg, 9 mg/kg, 9.5 mg/kg, 10 mg/kg 10.5 mg/kg, 11 mg/kg, 11.5 mg/kg, 12 mg/kg, 12.5 mg/kg, 13 mg/kg, 13.5 mg/kg, 14 mg/kg, 14.5 mg/kg, 15 mg/kg, 16 mg/kg, 17 mg/kg, 18 mg/kg, 19 mg/kg, 20 mg/kg, 21 mg/kg, 22 mg/kg, 23 mg/kg, 24 mg/kg, 25 mg/kg, 30 mg/kg, 35 mg/kg, 40 mg/kg, 45 mg/kg, 50 mg/kg, 55 mg/kg, 60 mg/kg, 65 mg/kg, 70 mg/kg, 75 mg/kg, 80 mg/kg, 85 mg/kg, 90 mg/kg, 95 mg/kg, 100 mg/kg, 105 mg/kg, 110 mg/kg, 115 mg/kg, 120 mg/kg, 125 mg/kg, 130 mg/kg, 135 mg/kg, 140 mg/kg, 145 mg/kg, 150 mg/kg, 155 mg/kg, 160 mg/kg, 165 mg/kg, 170 mg/kg, 175 mg/kg, 180 mg/kg, 185 mg/kg, 190 mg/kg, 195 mg/kg, 200 mg/kg, and any fraction or integer in between any two of the preceding amounts.

In some embodiments, a NOS inhibitor composition comprising L-NAME and/or a salt thereof, such as, for example, L-NAME hydrochloride, may be administered as a suitable dosage form, as described herein, to treat, ameliorate, and/or reduce the severity of glioblastoma or one or more symptoms thereof in a glioblastoma subject. In certain embodiments, an nNOS inhibitor composition comprising L-NAME and/or a salt thereof, such as, for example, L-NAME hydrochloride, may be administered to a glioblastoma subject to treat, ameliorate, and/or reduce the severity of glioblastoma or one or more symptoms thereof in the glioblastoma subject: (i) via a route selected from the group consisting of buccally, inhalation, intraarterially, intraarticularly, intracardially, intracerebroventricularly, intradermally, intramedullary, intramuscularly, intraperitoneally, intrathecally, intravenously, intraventricularly, nasally, ophthalmically, orally, rectally, sublabially, sublingually, subcutaneously, topically, and transdermally; and/or (ii) with a frequency selected from the group consisting of four times daily, three times daily, twice daily, once daily, once every two days, once every three days, once every four days, once every five days, once every six days, once a week, once every eight days, once every nine days, once every ten days, once every two weeks, once every three weeks, and once a month; and/or (iii) at a dosage (based on subject weight) selected from the group consisting of 0.1 μg/kg, 0.5 μg/kg, 1 μg/kg, 1.5 μg/kg, 2.0 μg/kg, 2.5 μg/kg, 3.0 μg/kg, 3.5 μg/kg, 4.0 μg/kg, 4.5 μg/kg, 5.0 μg/kg, 10 μg/kg, 15 μg/kg, 20 μg/kg, 25 μg/kg, 30 μg/kg, 35 μg/kg, 40 μg/kg, 45 μg/kg, 50 μg/kg, 55 μg/kg, 60 μg/kg, 65 μg/kg, 70 μg/kg, 75 μg/kg, 80 μg/kg, 85 μg/kg, 90 μg/kg, 95 μg/kg, 100 μg/kg, 150 μg/kg, 200 μg/kg, 250 μg/kg, 300 μg/kg, 350 μg/kg, 400 μg/kg, 450 μg/kg, 500 μg/kg, 550 μg/kg, 600 μg/kg, 650 μg/kg, 700 μg/kg, 750 μg/kg, 800 μg/kg, 850 μg/kg, 900 μg/kg, 1 mg/kg, 1.5 mg/kg, 2.0 mg/kg, 2.5 mg/kg, 3 mg/kg, 3.5 mg/kg, 4.0 mg/kg, 4.5 mg/kg, 5 mg/kg, 5.5 mg/kg, 6 mg/kg, 6.5 mg/kg, 7 mg/kg, 7.5 mg/kg, 8 mg/kg, 8.5 mg/kg, 9 mg/kg, 9.5 mg/kg, 10 mg/kg 10.5 mg/kg, 11 mg/kg, 11.5 mg/kg, 12 mg/kg, 12.5 mg/kg, 13 mg/kg, 13.5 mg/kg, 14 mg/kg, 14.5 mg/kg, 15 mg/kg, 16 mg/kg, 17 mg/kg, 18 mg/kg, 19 mg/kg, 20 mg/kg, 21 mg/kg, 22 mg/kg, 23 mg/kg, 24 mg/kg, 25 mg/kg, 30 mg/kg, 35 mg/kg, 40 mg/kg, 45 mg/kg, 50 mg/kg, 55 mg/kg, 60 mg/kg, 65 mg/kg, 70 mg/kg, 75 mg/kg, 80 mg/kg, 85 mg/kg, 90 mg/kg, 95 mg/kg, 100 mg/kg, 105 mg/kg, 110 mg/kg, 115 mg/kg, 120 mg/kg, 125 mg/kg, 130 mg/kg, 135 mg/kg, 140 mg/kg, 145 mg/kg, 150 mg/kg, 155 mg/kg, 160 mg/kg, 165 mg/kg, 170 mg/kg, 175 mg/kg, 180 mg/kg, 185 mg/kg, 190 mg/kg, 195 mg/kg, 200 mg/kg, and any fraction or integer in between any two of the preceding amounts.

In certain embodiments, a NOS inhibitor composition comprising L-NMMA and/or a salt thereof, such as, for example, L-NMMA acetate, may be administered as a suitable dosage form, as described herein, to treat, ameliorate, and/or reduce the severity of glioblastoma or one or more symptoms thereof in a glioblastoma subject. In some embodiments, an nNOS inhibitor composition comprising L-NMMA and/or a salt thereof, such as, for example, L-NMMA acetate, may be administered to a glioblastoma subject to treat, ameliorate, and/or reduce the severity of glioblastoma or one or more symptoms thereof in the glioblastoma subject: (i) via a route selected from the group consisting of buccally, inhalation, intraarterially, intraarticularly, intracardially, intracerebroventricularly, intradermally, intramedullary, intramuscularly, intraperitoneally, intrathecally, intravenously, intraventricularly, nasally, ophthalmically, orally, rectally, sublabially, sublingually, subcutaneously, topically, and transdermally; and/or (ii) with a frequency selected from the group consisting of four times daily, three times daily, twice daily, once daily, once every two days, once every three days, once every four days, once every five days, once every six days, once a week, once every eight days, once every nine days, once every ten days, once every two weeks, once every three weeks, and once a month; and/or (iii) at a dosage (based on subject weight) selected from the group consisting of 0.1 μg/kg, 0.5 μg/kg, 1 μg/kg, 1.5 μg/kg, 2.0 μg/kg, 2.5 μg/kg, 3.0 μg/kg, 3.5 μg/kg, 4.0 μg/kg, 4.5 μg/kg, 5.0 μg/kg, 10 μg/kg, 15 μg/kg, 20 μg/kg, 25 μg/kg, 30 μg/kg, 35 μg/kg, 40 μg/kg, 45 μg/kg, 50 μg/kg, 55 μg/kg, 60 μg/kg, 65 μg/kg, 70 μg/kg, 75 μg/kg, 80 μg/kg, 85 μg/kg, 90 μg/kg, 95 μg/kg, 100 μg/kg, 150 μg/kg, 200 μg/kg, 250 μg/kg, 300 μg/kg, 350 μg/kg, 400 μg/kg, 450 μg/kg, 500 μg/kg, 550 μg/kg, 600 μg/kg, 650 μg/kg, 700 μg/kg, 750 μg/kg, 800 μg/kg, 850 μg/kg, 900 μg/kg, 1 mg/kg, 1.5 mg/kg, 2.0 mg/kg, 2.5 mg/kg, 3 mg/kg, 3.5 mg/kg, 4.0 mg/kg, 4.5 mg/kg, 5 mg/kg, 5.5 mg/kg, 6 mg/kg, 6.5 mg/kg, 7 mg/kg, 7.5 mg/kg, 8 mg/kg, 8.5 mg/kg, 9 mg/kg, 9.5 mg/kg, 10 mg/kg 10.5 mg/kg, 11 mg/kg, 11.5 mg/kg, 12 mg/kg, 12.5 mg/kg, 13 mg/kg, 13.5 mg/kg, 14 mg/kg, 14.5 mg/kg, 15 mg/kg, 16 mg/kg, 17 mg/kg, 18 mg/kg, 19 mg/kg, 20 mg/kg, 21 mg/kg, 22 mg/kg, 23 mg/kg, 24 mg/kg, 25 mg/kg, 30 mg/kg, 35 mg/kg, 40 mg/kg, 45 mg/kg, 50 mg/kg, 55 mg/kg, 60 mg/kg, 65 mg/kg, 70 mg/kg, 75 mg/kg, 80 mg/kg, 85 mg/kg, 90 mg/kg, 95 mg/kg, 100 mg/kg, 105 mg/kg, 110 mg/kg, 115 mg/kg, 120 mg/kg, 125 mg/kg, 130 mg/kg, 135 mg/kg, 140 mg/kg, 145 mg/kg, 150 mg/kg, 155 mg/kg, 160 mg/kg, 165 mg/kg, 170 mg/kg, 175 mg/kg, 180 mg/kg, 185 mg/kg, 190 mg/kg, 195 mg/kg, 200 mg/kg, and any fraction or integer in between any two of the preceding amounts.

In some embodiments, a NOS inhibitor composition comprising GW-274150 and/or a salt thereof, such as, for example, GW-274150 phosphate, may be administered as a suitable dosage form, as described herein, to treat, ameliorate, and/or reduce the severity of glioblastoma or one or more symptoms thereof in a glioblastoma subject. In certain embodiments, an nNOS inhibitor composition comprising GW-274150 and/or a salt thereof, such as, for example, GW-274150 phosphate, may be administered to a glioblastoma subject to treat, ameliorate, and/or reduce the severity of glioblastoma or one or more symptoms thereof in the glioblastoma subject: (i) via a route selected from the group consisting of buccally, inhalation, intraarterially, intraarticularly, intracardially, intracerebroventricularly, intradermally, intramedullary, intramuscularly, intraperitoneally, intrathecally, intravenously, intraventricularly, nasally, ophthalmically, orally, rectally, sublabially, sublingually, subcutaneously, topically, and transdermally; and/or (ii) with a frequency selected from the group consisting of four times daily, three times daily, twice daily, once daily, once every two days, once every three days, once every four days, once every five days, once every six days, once a week, once every eight days, once every nine days, once every ten days, once every two weeks, once every three weeks, and once a month; and/or (iii) at a dosage (based on subject weight) selected from the group consisting of 0.1 μg/kg, 0.5 μg/kg, 1 μg/kg, 1.5 μg/kg, 2.0 μg/kg, 2.5 μg/kg, 3.0 μg/kg, 3.5 μg/kg, 4.0 μg/kg, 4.5 μg/kg, 5.0 μg/kg, 10 μg/kg, 15 μg/kg, 20 μg/kg, 25 μg/kg, 30 μg/kg, 35 μg/kg, 40 μg/kg, 45 μg/kg, 50 μg/kg, 55 μg/kg, 60 μg/kg, 65 μg/kg, 70 μg/kg, 75 μg/kg, 80 μg/kg, 85 μg/kg, 90 μg/kg, 95 μg/kg, 100 μg/kg, 150 μg/kg, 200 μg/kg, 250 μg/kg, 300 μg/kg, 350 μg/kg, 400 μg/kg, 450 μg/kg, 500 μg/kg, 550 μg/kg, 600 μg/kg, 650 μg/kg, 700 μg/kg, 750 μg/kg, 800 μg/kg, 850 μg/kg, 900 μg/kg, 1 mg/kg, 1.5 mg/kg, 2.0 mg/kg, 2.5 mg/kg, 3 mg/kg, 3.5 mg/kg, 4.0 mg/kg, 4.5 mg/kg, 5 mg/kg, 5.5 mg/kg, 6 mg/kg, 6.5 mg/kg, 7 mg/kg, 7.5 mg/kg, 8 mg/kg, 8.5 mg/kg, 9 mg/kg, 9.5 mg/kg, 10 mg/kg 10.5 mg/kg, 11 mg/kg, 11.5 mg/kg, 12 mg/kg, 12.5 mg/kg, 13 mg/kg, 13.5 mg/kg, 14 mg/kg, 14.5 mg/kg, 15 mg/kg, 16 mg/kg, 17 mg/kg, 18 mg/kg, 19 mg/kg, 20 mg/kg, 21 mg/kg, 22 mg/kg, 23 mg/kg, 24 mg/kg, 25 mg/kg, 30 mg/kg, 35 mg/kg, 40 mg/kg, 45 mg/kg, 50 mg/kg, 55 mg/kg, 60 mg/kg, 65 mg/kg, 70 mg/kg, 75 mg/kg, 80 mg/kg, 85 mg/kg, 90 mg/kg, 95 mg/kg, 100 mg/kg, 105 mg/kg, 110 mg/kg, 115 mg/kg, 120 mg/kg, 125 mg/kg, 130 mg/kg, 135 mg/kg, 140 mg/kg, 145 mg/kg, 150 mg/kg, 155 mg/kg, 160 mg/kg, 165 mg/kg, 170 mg/kg, 175 mg/kg, 180 mg/kg, 185 mg/kg, 190 mg/kg, 195 mg/kg, 200 mg/kg, and any fraction or integer in between any two of the preceding amounts.

In certain embodiments, a NOS inhibitor composition comprising L-NIL and/or a salt thereof, such as, for example, L-NIL hydrochloride, may be administered as a suitable dosage form, as described herein, to treat, ameliorate, and/or reduce the severity of glioblastoma or one or more symptoms thereof in a glioblastoma subject. In some embodiments, an nNOS inhibitor composition comprising L-NIL and/or a salt thereof, such as, for example, L-NIL hydrochloride, may be administered to a glioblastoma subject to treat, ameliorate, and/or reduce the severity of glioblastoma or one or more symptoms thereof in the glioblastoma subject: (i) via a route selected from the group consisting of buccally, inhalation, intraarterially, intraarticularly, intracardially, intracerebroventricularly, intradermally, intramedullary, intramuscularly, intraperitoneally, intrathecally, intravenously, intraventricularly, nasally, ophthalmically, orally, rectally, sublabially, sublingually, subcutaneously, topically, and transdermally; and/or (ii) with a frequency selected from the group consisting of four times daily, three times daily, twice daily, once daily, once every two days, once every three days, once every four days, once every five days, once every six days, once a week, once every eight days, once every nine days, once every ten days, once every two weeks, once every three weeks, and once a month; and/or (iii) at a dosage (based on subject weight) selected from the group consisting of 0.1 μg/kg, 0.5 μg/kg, 1 g/kg, 1.5 μg/kg, 2.0 μg/kg, 2.5 μg/kg, 3.0 μg/kg, 3.5 μg/kg, 4.0 μg/kg, 4.5 μg/kg, 5.0 μg/kg, 10 μg/kg, 15 μg/kg, 20 μg/kg, 25 μg/kg, 30 μg/kg, 35 μg/kg, 40 μg/kg, 45 μg/kg, 50 μg/kg, 55 μg/kg, 60 μg/kg, 65 μg/kg, 70 μg/kg, 75 μg/kg, 80 μg/kg, 85 μg/kg, 90 μg/kg, 95 μg/kg, 100 μg/kg, 150 μg/kg, 200 μg/kg, 250 μg/kg, 300 μg/kg, 350 μg/kg, 400 μg/kg, 450 μg/kg, 500 μg/kg, 550 μg/kg, 600 μg/kg, 650 μg/kg, 700 μg/kg, 750 μg/kg, 800 μg/kg, 850 μg/kg, 900 μg/kg, 1 mg/kg, 1.5 mg/kg, 2.0 mg/kg, 2.5 mg/kg, 3 mg/kg, 3.5 mg/kg, 4.0 mg/kg, 4.5 mg/kg, 5 mg/kg, 5.5 mg/kg, 6 mg/kg, 6.5 mg/kg, 7 mg/kg, 7.5 mg/kg, 8 mg/kg, 8.5 mg/kg, 9 mg/kg, 9.5 mg/kg, 10 mg/kg 10.5 mg/kg, 11 mg/kg, 11.5 mg/kg, 12 mg/kg, 12.5 mg/kg, 13 mg/kg, 13.5 mg/kg, 14 mg/kg, 14.5 mg/kg, 15 mg/kg, 16 mg/kg, 17 mg/kg, 18 mg/kg, 19 mg/kg, 20 mg/kg, 21 mg/kg, 22 mg/kg, 23 mg/kg, 24 mg/kg, 25 mg/kg, 30 mg/kg, 35 mg/kg, 40 mg/kg, 45 mg/kg, 50 mg/kg, 55 mg/kg, 60 mg/kg, 65 mg/kg, 70 mg/kg, 75 mg/kg, 80 mg/kg, 85 mg/kg, 90 mg/kg, 95 mg/kg, 100 mg/kg, 105 mg/kg, 110 mg/kg, 115 mg/kg, 120 mg/kg, 125 mg/kg, 130 mg/kg, 135 mg/kg, 140 mg/kg, 145 mg/kg, 150 mg/kg, 155 mg/kg, 160 mg/kg, 165 mg/kg, 170 mg/kg, 175 mg/kg, 180 mg/kg, 185 mg/kg, 190 mg/kg, 195 mg/kg, 200 mg/kg, and any fraction or integer in between any two of the preceding amounts.

In some embodiments, a NOS inhibitor composition comprising 1400W and/or a salt thereof, such as, for example, 1400W dihydrochloride, may be administered as a suitable dosage form, as described herein, to treat, ameliorate, and/or reduce the severity of glioblastoma or one or more symptoms thereof in a glioblastoma subject. In certain embodiments, an nNOS inhibitor composition comprising 1400W and/or a salt thereof, such as, for example, 1400W dihydrochloride, may be administered to a glioblastoma subject to treat, ameliorate, and/or reduce the severity of glioblastoma or one or more symptoms thereof in the glioblastoma subject: (i) via a route selected from the group consisting of buccally, inhalation, intraarterially, intraarticularly, intracardially, intracerebroventricularly, intradermally, intramedullary, intramuscularly, intraperitoneally, intrathecally, intravenously, intraventricularly, nasally, ophthalmically, orally, rectally, sublabially, sublingually, subcutaneously, topically, and transdermally; and/or (ii) with a frequency selected from the group consisting of four times daily, three times daily, twice daily, once daily, once every two days, once every three days, once every four days, once every five days, once every six days, once a week, once every eight days, once every nine days, once every ten days, once every two weeks, once every three weeks, and once a month; and/or (iii) at a dosage (based on subject weight) selected from the group consisting of 0.1 μg/kg, 0.5 μg/kg, 1 μg/kg, 1.5 μg/kg, 2.0 μg/kg, 2.5 μg/kg, 3.0 μg/kg, 3.5 μg/kg, 4.0 μg/kg, 4.5 μg/kg, 5.0 μg/kg, 10 μg/kg, 15 μg/kg, 20 μg/kg, 25 μg/kg, 30 μg/kg, 35 μg/kg, 40 μg/kg, 45 μg/kg, 50 μg/kg, 55 μg/kg, 60 μg/kg, 65 μg/kg, 70 μg/kg, 75 μg/kg, 80 μg/kg, 85 μg/kg, 90 μg/kg, 95 μg/kg, 100 μg/kg, 150 μg/kg, 200 μg/kg, 250 μg/kg, 300 μg/kg, 350 μg/kg, 400 μg/kg, 450 μg/kg, 500 μg/kg, 550 μg/kg, 600 μg/kg, 650 μg/kg, 700 μg/kg, 750 μg/kg, 800 μg/kg, 850 μg/kg, 900 μg/kg, 1 mg/kg, 1.5 mg/kg, 2.0 mg/kg, 2.5 mg/kg, 3 mg/kg, 3.5 mg/kg, 4.0 mg/kg, 4.5 mg/kg, 5 mg/kg, 5.5 mg/kg, 6 mg/kg, 6.5 mg/kg, 7 mg/kg, 7.5 mg/kg, 8 mg/kg, 8.5 mg/kg, 9 mg/kg, 9.5 mg/kg, 10 mg/kg 10.5 mg/kg, 11 mg/kg, 11.5 mg/kg, 12 mg/kg, 12.5 mg/kg, 13 mg/kg, 13.5 mg/kg, 14 mg/kg, 14.5 mg/kg, 15 mg/kg, 16 mg/kg, 17 mg/kg, 18 mg/kg, 19 mg/kg, 20 mg/kg, 21 mg/kg, 22 mg/kg, 23 mg/kg, 24 mg/kg, 25 mg/kg, 30 mg/kg, 35 mg/kg, 40 mg/kg, 45 mg/kg, 50 mg/kg, 55 mg/kg, 60 mg/kg, 65 mg/kg, 70 mg/kg, 75 mg/kg, 80 mg/kg, 85 mg/kg, 90 mg/kg, 95 mg/kg, 100 mg/kg, 105 mg/kg, 110 mg/kg, 115 mg/kg, 120 mg/kg, 125 mg/kg, 130 mg/kg, 135 mg/kg, 140 mg/kg, 145 mg/kg, 150 mg/kg, 155 mg/kg, 160 mg/kg, 165 mg/kg, 170 mg/kg, 175 mg/kg, 180 mg/kg, 185 mg/kg, 190 mg/kg, 195 mg/kg, 200 mg/kg, and any fraction or integer in between any two of the preceding amounts.

In certain embodiments, a NOS inhibitor composition comprising OKN-007 and/or a salt thereof, such as, for example, OKN-007 sodium, may be administered as a suitable dosage form, as described herein, to treat, ameliorate, and/or reduce the severity of glioblastoma or one or more symptoms thereof in a glioblastoma subject. In some embodiments, an nNOS inhibitor composition comprising OKN-007 and/or a salt thereof, such as, for example, OKN-007 sodium, may be administered to a glioblastoma subject to treat, ameliorate, and/or reduce the severity of glioblastoma or one or more symptoms thereof in the glioblastoma subject: (i) via a route selected from the group consisting of buccally, inhalation, intraarterially, intraarticularly, intracardially, intracerebroventricularly, intradermally, intramedullary, intramuscularly, intraperitoneally, intrathecally, intravenously, intraventricularly, nasally, ophthalmically, orally, rectally, sublabially, sublingually, subcutaneously, topically, and transdermally; and/or (ii) with a frequency selected from the group consisting of four times daily, three times daily, twice daily, once daily, once every two days, once every three days, once every four days, once every five days, once every six days, once a week, once every eight days, once every nine days, once every ten days, once every two weeks, once every three weeks, and once a month; and/or (iii) at a dosage (based on subject weight) selected from the group consisting of 0.1 μg/kg, 0.5 μg/kg, 1 μg/kg, 1.5 μg/kg, 2.0 μg/kg, 2.5 μg/kg, 3.0 μg/kg, 3.5 μg/kg, 4.0 μg/kg, 4.5 μg/kg, 5.0 μg/kg, 10 μg/kg, 15 μg/kg, 20 μg/kg, 25 μg/kg, 30 μg/kg, 35 μg/kg, 40 μg/kg, 45 μg/kg, 50 μg/kg, 55 μg/kg, 60 μg/kg, 65 μg/kg, 70 μg/kg, 75 μg/kg, 80 μg/kg, 85 μg/kg, 90 μg/kg, 95 μg/kg, 100 μg/kg, 150 μg/kg, 200 μg/kg, 250 μg/kg, 300 μg/kg, 350 μg/kg, 400 μg/kg, 450 μg/kg, 500 μg/kg, 550 μg/kg, 600 μg/kg, 650 μg/kg, 700 μg/kg, 750 μg/kg, 800 μg/kg, 850 μg/kg, 900 μg/kg, 1 mg/kg, 1.5 mg/kg, 2.0 mg/kg, 2.5 mg/kg, 3 mg/kg, 3.5 mg/kg, 4.0 mg/kg, 4.5 mg/kg, 5 mg/kg, 5.5 mg/kg, 6 mg/kg, 6.5 mg/kg, 7 mg/kg, 7.5 mg/kg, 8 mg/kg, 8.5 mg/kg, 9 mg/kg, 9.5 mg/kg, 10 mg/kg 10.5 mg/kg, 11 mg/kg, 11.5 mg/kg, 12 mg/kg, 12.5 mg/kg, 13 mg/kg, 13.5 mg/kg, 14 mg/kg, 14.5 mg/kg, 15 mg/kg, 16 mg/kg, 17 mg/kg, 18 mg/kg, 19 mg/kg, 20 mg/kg, 21 mg/kg, 22 mg/kg, 23 mg/kg, 24 mg/kg, 25 mg/kg, 30 mg/kg, 35 mg/kg, 40 mg/kg, 45 mg/kg, 50 mg/kg, 55 mg/kg, 60 mg/kg, 65 mg/kg, 70 mg/kg, 75 mg/kg, 80 mg/kg, 85 mg/kg, 90 mg/kg, 95 mg/kg, 100 mg/kg, 105 mg/kg, 110 mg/kg, 115 mg/kg, 120 mg/kg, 125 mg/kg, 130 mg/kg, 135 mg/kg, 140 mg/kg, 145 mg/kg, 150 mg/kg, 155 mg/kg, 160 mg/kg, 165 mg/kg, 170 mg/kg, 175 mg/kg, 180 mg/kg, 185 mg/kg, 190 mg/kg, 195 mg/kg, 200 mg/kg, and any fraction or integer in between any two of the preceding amounts.

In some embodiments, a NOS inhibitor composition comprising BYK-191023 and/or a salt thereof, such as, for example, BYK-191023 dihydrochloride, may be administered as a suitable dosage form, as described herein, to treat, ameliorate, and/or reduce the severity of glioblastoma or one or more symptoms thereof in a glioblastoma subject. In certain embodiments, an nNOS inhibitor composition comprising BYK-191023 and/or a salt thereof, such as, for example, BYK-191023 dihydrochloride, may be administered to a glioblastoma subject to treat, ameliorate, and/or reduce the severity of glioblastoma or one or more symptoms thereof in the glioblastoma subject: (i) via a route selected from the group consisting of buccally, inhalation, intraarterially, intraarticularly, intracardially, intracerebroventricularly, intradermally, intramedullary, intramuscularly, intraperitoneally, intrathecally, intravenously, intraventricularly, nasally, ophthalmically, orally, rectally, sublabially, sublingually, subcutaneously, topically, and transdermally; and/or (ii) with a frequency selected from the group consisting of four times daily, three times daily, twice daily, once daily, once every two days, once every three days, once every four days, once every five days, once every six days, once a week, once every eight days, once every nine days, once every ten days, once every two weeks, once every three weeks, and once a month; and/or (iii) at a dosage (based on subject weight) selected from the group consisting of 0.1 μg/kg, 0.5 μg/kg, 1 μg/kg, 1.5 μg/kg, 2.0 μg/kg, 2.5 μg/kg, 3.0 μg/kg, 3.5 μg/kg, 4.0 μg/kg, 4.5 μg/kg, 5.0 μg/kg, 10 μg/kg, 15 μg/kg, 20 μg/kg, 25 μg/kg, 30 μg/kg, 35 μg/kg, 40 μg/kg, 45 μg/kg, 50 μg/kg, 55 μg/kg, 60 μg/kg, 65 μg/kg, 70 μg/kg, 75 μg/kg, 80 μg/kg, 85 μg/kg, 90 μg/kg, 95 μg/kg, 100 μg/kg, 150 μg/kg, 200 μg/kg, 250 μg/kg, 300 μg/kg, 350 μg/kg, 400 μg/kg, 450 μg/kg, 500 μg/kg, 550 μg/kg, 600 μg/kg, 650 μg/kg, 700 μg/kg, 750 μg/kg, 800 μg/kg, 850 μg/kg, 900 μg/kg, 1 mg/kg, 1.5 mg/kg, 2.0 mg/kg, 2.5 mg/kg, 3 mg/kg, 3.5 mg/kg, 4.0 mg/kg, 4.5 mg/kg, 5 mg/kg, 5.5 mg/kg, 6 mg/kg, 6.5 mg/kg, 7 mg/kg, 7.5 mg/kg, 8 mg/kg, 8.5 mg/kg, 9 mg/kg, 9.5 mg/kg, 10 mg/kg 10.5 mg/kg, 11 mg/kg, 11.5 mg/kg, 12 mg/kg, 12.5 mg/kg, 13 mg/kg, 13.5 mg/kg, 14 mg/kg, 14.5 mg/kg, 15 mg/kg, 16 mg/kg, 17 mg/kg, 18 mg/kg, 19 mg/kg, 20 mg/kg, 21 mg/kg, 22 mg/kg, 23 mg/kg, 24 mg/kg, 25 mg/kg, 30 mg/kg, 35 mg/kg, 40 mg/kg, 45 mg/kg, 50 mg/kg, 55 mg/kg, 60 mg/kg, 65 mg/kg, 70 mg/kg, 75 mg/kg, 80 mg/kg, 85 mg/kg, 90 mg/kg, 95 mg/kg, 100 mg/kg, 105 mg/kg, 110 mg/kg, 115 mg/kg, 120 mg/kg, 125 mg/kg, 130 mg/kg, 135 mg/kg, 140 mg/kg, 145 mg/kg, 150 mg/kg, 155 mg/kg, 160 mg/kg, 165 mg/kg, 170 mg/kg, 175 mg/kg, 180 mg/kg, 185 mg/kg, 190 mg/kg, 195 mg/kg, 200 mg/kg, and any fraction or integer in between any two of the preceding amounts.

In certain embodiments, a NOS inhibitor composition comprising CM544 and/or a salt thereof, such as, for example, CM544 dihydrochloride, may be administered as a suitable dosage form, as described herein, to treat, ameliorate, and/or reduce the severity of glioblastoma or one or more symptoms thereof in a glioblastoma subject. In some embodiments, an nNOS inhibitor composition comprising CM544 and/or a salt thereof, such as, for example, CM544 dihydrochloride, may be administered to a glioblastoma subject to treat, ameliorate, and/or reduce the severity of glioblastoma or one or more symptoms thereof in the glioblastoma subject: (i) via a route selected from the group consisting of buccally, inhalation, intraarterially, intraarticularly, intracardially, intracerebroventricularly, intradermally, intramedullary, intramuscularly, intraperitoneally, intrathecally, intravenously, intraventricularly, nasally, ophthalmically, orally, rectally, sublabially, sublingually, subcutaneously, topically, and transdermally; and/or (ii) with a frequency selected from the group consisting of four times daily, three times daily, twice daily, once daily, once every two days, once every three days, once every four days, once every five days, once every six days, once a week, once every eight days, once every nine days, once every ten days, once every two weeks, once every three weeks, and once a month; and/or (iii) at a dosage (based on subject weight) selected from the group consisting of 0.1 μg/kg, 0.5 μg/kg, 1 μg/kg, 1.5 μg/kg, 2.0 μg/kg, 2.5 μg/kg, 3.0 μg/kg, 3.5 μg/kg, 4.0 μg/kg, 4.5 μg/kg, 5.0 μg/kg, 10 μg/kg, 15 μg/kg, 20 μg/kg, 25 μg/kg, 30 μg/kg, 35 μg/kg, 40 μg/kg, 45 μg/kg, 50 μg/kg, 55 μg/kg, 60 μg/kg, 65 μg/kg, 70 μg/kg, 75 μg/kg, 80 μg/kg, 85 μg/kg, 90 μg/kg, 95 μg/kg, 100 μg/kg, 150 μg/kg, 200 μg/kg, 250 μg/kg, 300 μg/kg, 350 μg/kg, 400 μg/kg, 450 μg/kg, 500 μg/kg, 550 μg/kg, 600 μg/kg, 650 μg/kg, 700 μg/kg, 750 μg/kg, 800 μg/kg, 850 μg/kg, 900 μg/kg, 1 mg/kg, 1.5 mg/kg, 2.0 mg/kg, 2.5 mg/kg, 3 mg/kg, 3.5 mg/kg, 4.0 mg/kg, 4.5 mg/kg, 5 mg/kg, 5.5 mg/kg, 6 mg/kg, 6.5 mg/kg, 7 mg/kg, 7.5 mg/kg, 8 mg/kg, 8.5 mg/kg, 9 mg/kg, 9.5 mg/kg, 10 mg/kg 10.5 mg/kg, 11 mg/kg, 11.5 mg/kg, 12 mg/kg, 12.5 mg/kg, 13 mg/kg, 13.5 mg/kg, 14 mg/kg, 14.5 mg/kg, 15 mg/kg, 16 mg/kg, 17 mg/kg, 18 mg/kg, 19 mg/kg, 20 mg/kg, 21 mg/kg, 22 mg/kg, 23 mg/kg, 24 mg/kg, 25 mg/kg, 30 mg/kg, 35 mg/kg, 40 mg/kg, 45 mg/kg, 50 mg/kg, 55 mg/kg, 60 mg/kg, 65 mg/kg, 70 mg/kg, 75 mg/kg, 80 mg/kg, 85 mg/kg, 90 mg/kg, 95 mg/kg, 100 mg/kg, 105 mg/kg, 110 mg/kg, 115 mg/kg, 120 mg/kg, 125 mg/kg, 130 mg/kg, 135 mg/kg, 140 mg/kg, 145 mg/kg, 150 mg/kg, 155 mg/kg, 160 mg/kg, 165 mg/kg, 170 mg/kg, 175 mg/kg, 180 mg/kg, 185 mg/kg, 190 mg/kg, 195 mg/kg, 200 mg/kg, and any fraction or integer in between any two of the preceding amounts.

In certain embodiments, an nNOS inhibitor composition comprising one or more dsRNAs each comprising at least 30 base pairs, may be administered as a suitable dosage form, as described herein, to treat, ameliorate, and/or reduce the severity of glioblastoma or one or more symptoms thereof in a glioblastoma subject. In some embodiments, an nNOS inhibitor composition comprising one or more dsRNAs, each comprising at least 30 base pairs, may be administered to a glioblastoma subject to treat, ameliorate, and/or reduce the severity of glioblastoma or one or more symptoms thereof in the glioblastoma subject: (i) via a route selected from the group consisting of buccally, inhalation, intraarterially, intraarticularly, intracardially, intracerebroventricularly, intradermally, intramedullary, intramuscularly, intraperitoneally, intrathecally, intravenously, intraventricularly, nasally, ophthalmically, orally, rectally, sublabially, sublingually, subcutaneously, topically, and transdermally; and/or (ii) with a frequency selected from the group consisting of four times daily, three times daily, twice daily, once daily, once every two days, once every three days, once every four days, once every five days, once every six days, once a week, once every eight days, once every nine days, once every ten days, once every two weeks, once every three weeks, and once a month; and/or (iii) at a dosage (based on subject weight) selected from the group consisting of 0.1 μg/kg, 0.5 μg/kg, 1 μg/kg, 1.5 μg/kg, 2.0 μg/kg, 2.5 μg/kg, 3.0 μg/kg, 3.5 μg/kg, 4.0 μg/kg, 4.5 μg/kg, 5.0 μg/kg, 10 μg/kg, 15 μg/kg, 20 μg/kg, 25 μg/kg, 30 μg/kg, 35 μg/kg, 40 μg/kg, 45 μg/kg, 50 μg/kg, 55 μg/kg, 60 μg/kg, 65 μg/kg, 70 μg/kg, 75 μg/kg, 80 μg/kg, 85 μg/kg, 90 μg/kg, 95 μg/kg, 100 μg/kg, 150 μg/kg, 200 μg/kg, 250 μg/kg, 300 μg/kg, 350 μg/kg, 400 μg/kg, 450 μg/kg, 500 μg/kg, 550 μg/kg, 600 μg/kg, 650 μg/kg, 700 μg/kg, 750 μg/kg, 800 μg/kg, 850 μg/kg, 900 μg/kg, 1 mg/kg, 1.5 mg/kg, 2.0 mg/kg, 2.5 mg/kg, 3 mg/kg, 3.5 mg/kg, 4.0 mg/kg, 4.5 mg/kg, 5 mg/kg, 5.5 mg/kg, 6 mg/kg, 6.5 mg/kg, 7 mg/kg, 7.5 mg/kg, 8 mg/kg, 8.5 mg/kg, 9 mg/kg, 9.5 mg/kg, 10 mg/kg 10.5 mg/kg, 11 mg/kg, 11.5 mg/kg, 12 mg/kg, 12.5 mg/kg, 13 mg/kg, 13.5 mg/kg, 14 mg/kg, 14.5 mg/kg, 15 mg/kg, 16 mg/kg, 17 mg/kg, 18 mg/kg, 19 mg/kg, 20 mg/kg, 21 mg/kg, 22 mg/kg, 23 mg/kg, 24 mg/kg, 25 mg/kg, 30 mg/kg, 35 mg/kg, 40 mg/kg, 45 mg/kg, 50 mg/kg, 55 mg/kg, 60 mg/kg, 65 mg/kg, 70 mg/kg, 75 mg/kg, 80 mg/kg, 85 mg/kg, 90 mg/kg, 95 mg/kg, 100 mg/kg, 105 mg/kg, 110 mg/kg, 115 mg/kg, 120 mg/kg, 125 mg/kg, 130 mg/kg, 135 mg/kg, 140 mg/kg, 145 mg/kg, 150 mg/kg, 155 mg/kg, 160 mg/kg, 165 mg/kg, 170 mg/kg, 175 mg/kg, 180 mg/kg, 185 mg/kg, 190 mg/kg, 195 mg/kg, 200 mg/kg, and any fraction or integer in between any two of the preceding amounts.

In some embodiments, an nNOS inhibitor composition comprising one or more siRNAs, may be administered as a suitable dosage form, as described herein, to treat, ameliorate, and/or reduce the severity of glioblastoma or one or more symptoms thereof in a glioblastoma subject. In certain embodiments, an nNOS inhibitor composition comprising one or more siRNAs selected from the group consisting of SEQ ID NO. 4, SEQ ID NO. 5, SEQ ID NO. 6, SEQ ID NO. 7, SEQ ID NO. 8, and any combination thereof, may be administered as a suitable dosage form, as described herein, to treat, ameliorate, and/or reduce the severity of glioblastoma or one or more symptoms thereof in a glioblastoma subject. In some embodiments, an nNOS inhibitor composition comprising one or more siRNAs selected from the group consisting of SEQ ID NO. 4, SEQ ID NO. 5, SEQ ID NO. 6, SEQ ID NO. 7, SEQ ID NO. 8, and any combination thereof, may be administered to a glioblastoma subject to treat, ameliorate, and/or reduce the severity of glioblastoma or one or more symptoms thereof in the glioblastoma subject: (i) via a route selected from the group consisting of buccally, inhalation, intraarterially, intraarticularly, intracardially, intracerebroventricularly, intradermally, intramedullary, intramuscularly, intraperitoneally, intrathecally, intravenously, intraventricularly, nasally, ophthalmically, orally, rectally, sublabially, sublingually, subcutaneously, topically, and transdermally; and/or (ii) with a frequency selected from the group consisting of four times daily, three times daily, twice daily, once daily, once every two days, once every three days, once every four days, once every five days, once every six days, once a week, once every eight days, once every nine days, once every ten days, once every two weeks, once every three weeks, and once a month; and/or (iii) at a dosage (based on subject weight) selected from the group consisting of 0.1 μg/kg, 0.5 μg/kg, 1 μg/kg, 1.5 μg/kg, 2.0 μg/kg, 2.5 μg/kg, 3.0 μg/kg, 3.5 μg/kg, 4.0 μg/kg, 4.5 μg/kg, 5.0 μg/kg, 10 μg/kg, 15 μg/kg, 20 μg/kg, 25 μg/kg, 30 μg/kg, 35 μg/kg, 40 μg/kg, 45 μg/kg, 50 μg/kg, 55 μg/kg, 60 μg/kg, 65 μg/kg, 70 μg/kg, 75 μg/kg, 80 μg/kg, 85 μg/kg, 90 μg/kg, 95 μg/kg, 100 μg/kg, 150 μg/kg, 200 μg/kg, 250 μg/kg, 300 μg/kg, 350 μg/kg, 400 μg/kg, 450 μg/kg, 500 μg/kg, 550 μg/kg, 600 μg/kg, 650 μg/kg, 700 μg/kg, 750 μg/kg, 800 μg/kg, 850 μg/kg, 900 μg/kg, 1 mg/kg, 1.5 mg/kg, 2.0 mg/kg, 2.5 mg/kg, 3 mg/kg, 3.5 mg/kg, 4.0 mg/kg, 4.5 mg/kg, 5 mg/kg, 5.5 mg/kg, 6 mg/kg, 6.5 mg/kg, 7 mg/kg, 7.5 mg/kg, 8 mg/kg, 8.5 mg/kg, 9 mg/kg, 9.5 mg/kg, 10 mg/kg 10.5 mg/kg, 11 mg/kg, 11.5 mg/kg, 12 mg/kg, 12.5 mg/kg, 13 mg/kg, 13.5 mg/kg, 14 mg/kg, 14.5 mg/kg, 15 mg/kg, 16 mg/kg, 17 mg/kg, 18 mg/kg, 19 mg/kg, 20 mg/kg, 21 mg/kg, 22 mg/kg, 23 mg/kg, 24 mg/kg, 25 mg/kg, 30 mg/kg, 35 mg/kg, 40 mg/kg, 45 mg/kg, 50 mg/kg, 55 mg/kg, 60 mg/kg, 65 mg/kg, 70 mg/kg, 75 mg/kg, 80 mg/kg, 85 mg/kg, 90 mg/kg, 95 mg/kg, 100 mg/kg, 105 mg/kg, 110 mg/kg, 115 mg/kg, 120 mg/kg, 125 mg/kg, 130 mg/kg, 135 mg/kg, 140 mg/kg, 145 mg/kg, 150 mg/kg, 155 mg/kg, 160 mg/kg, 165 mg/kg, 170 mg/kg, 175 mg/kg, 180 mg/kg, 185 mg/kg, 190 mg/kg, 195 mg/kg, 200 mg/kg, and any fraction or integer in between any two of the preceding amounts.

In some embodiments, an nNOS inhibitor composition comprising one or more shRNAs, may be administered as a suitable dosage form, as described herein, to treat, ameliorate, and/or reduce the severity of glioblastoma or one or more symptoms thereof in a glioblastoma subject. In certain embodiments, an nNOS inhibitor composition comprising one or more shRNAs selected from the group consisting of SEQ ID NO. 9, SEQ ID NO. 10, SEQ ID NO. 11, SEQ ID NO. 12, SEQ ID NO. 13, SEQ ID NO. 14, and any combination thereof, may be administered as a suitable dosage form, as described herein, to treat, ameliorate, and/or reduce the severity of glioblastoma or one or more symptoms thereof in a glioblastoma subject. In some embodiments, an nNOS inhibitor composition comprising one or more shRNAs selected from the group consisting of SEQ ID NO. 9, SEQ ID NO. 10, SEQ ID NO. 11, SEQ ID NO. 12, SEQ ID NO. 13, SEQ ID NO. 14, and any combination thereof, may be administered to a glioblastoma subject to treat, ameliorate, and/or reduce the severity of glioblastoma or one or more symptoms thereof in the glioblastoma subject: (i) via a route selected from the group consisting of buccally, inhalation, intraarterially, intraarticularly, intracardially, intracerebroventricularly, intradermally, intramedullary, intramuscularly, intraperitoneally, intrathecally, intravenously, intraventricularly, nasally, ophthalmically, orally, rectally, sublabially, sublingually, subcutaneously, topically, and transdermally; and/or (ii) with a frequency selected from the group consisting of four times daily, three times daily, twice daily, once daily, once every two days, once every three days, once every four days, once every five days, once every six days, once a week, once every eight days, once every nine days, once every ten days, once every two weeks, once every three weeks, and once a month; and/or (iii) at a dosage (based on subject weight) selected from the group consisting of 0.1 μg/kg, 0.5 μg/kg, 1 μg/kg, 1.5 μg/kg, 2.0 μg/kg, 2.5 μg/kg, 3.0 μg/kg, 3.5 μg/kg, 4.0 μg/kg, 4.5 μg/kg, 5.0 μg/kg, 10 μg/kg, 15 μg/kg, 20 μg/kg, 25 μg/kg, 30 μg/kg, 35 μg/kg, 40 μg/kg, 45 μg/kg, 50 μg/kg, 55 μg/kg, 60 μg/kg, 65 μg/kg, 70 μg/kg, 75 μg/kg, 80 μg/kg, 85 μg/kg, 90 μg/kg, 95 μg/kg, 100 μg/kg, 150 μg/kg, 200 μg/kg, 250 μg/kg, 300 μg/kg, 350 μg/kg, 400 μg/kg, 450 μg/kg, 500 μg/kg, 550 μg/kg, 600 μg/kg, 650 μg/kg, 700 μg/kg, 750 μg/kg, 800 μg/kg, 850 μg/kg, 900 μg/kg, 1 mg/kg, 1.5 mg/kg, 2.0 mg/kg, 2.5 mg/kg, 3 mg/kg, 3.5 mg/kg, 4.0 mg/kg, 4.5 mg/kg, 5 mg/kg, 5.5 mg/kg, 6 mg/kg, 6.5 mg/kg, 7 mg/kg, 7.5 mg/kg, 8 mg/kg, 8.5 mg/kg, 9 mg/kg, 9.5 mg/kg, 10 mg/kg 10.5 mg/kg, 11 mg/kg, 11.5 mg/kg, 12 mg/kg, 12.5 mg/kg, 13 mg/kg, 13.5 mg/kg, 14 mg/kg, 14.5 mg/kg, 15 mg/kg, 16 mg/kg, 17 mg/kg, 18 mg/kg, 19 mg/kg, 20 mg/kg, 21 mg/kg, 22 mg/kg, 23 mg/kg, 24 mg/kg, 25 mg/kg, 30 mg/kg, 35 mg/kg, 40 mg/kg, 45 mg/kg, 50 mg/kg, 55 mg/kg, 60 mg/kg, 65 mg/kg, 70 mg/kg, 75 mg/kg, 80 mg/kg, 85 mg/kg, 90 mg/kg, 95 mg/kg, 100 mg/kg, 105 mg/kg, 110 mg/kg, 115 mg/kg, 120 mg/kg, 125 mg/kg, 130 mg/kg, 135 mg/kg, 140 mg/kg, 145 mg/kg, 150 mg/kg, 155 mg/kg, 160 mg/kg, 165 mg/kg, 170 mg/kg, 175 mg/kg, 180 mg/kg, 185 mg/kg, 190 mg/kg, 195 mg/kg, 200 mg/kg, and any fraction or integer in between any two of the preceding amounts.

In some embodiments, an nNOS inhibitor composition comprising one or more miRNAs, may be administered as a suitable dosage form, as described herein, to treat, ameliorate, and/or reduce the severity of glioblastoma or one or more symptoms thereof in a glioblastoma subject. In certain embodiments, an nNOS inhibitor composition comprising one or more miRNAs selected from the group consisting of miR-146a, hsa-miR-146a-5p, hsa-miR-1273f, hsa-miR-143-3p, hsa-miR-147a, hsa-miR-3622a-5p, hsa-miR-3911, hsa-miR-4269, hsa-miR-4708-5p, hsa-miR-4710, hsa-miR-4770, hsa-miR-4792, hsa-miR-574-5p, hsa-miR-6088, hsa-miR-644a, hsa-miR-6715b-5p, hsa-miR-6768-5p, hsa-miR-6867-5p, hsa-miR-7855-5p, hsa-miR-8485, and any combination thereof, may be administered as a suitable dosage form, as described herein, to treat, ameliorate, and/or reduce the severity of glioblastoma or one or more symptoms thereof in a glioblastoma subject. In some embodiments, an nNOS inhibitor composition comprising one or more miRNAs selected from the group consisting of miR-146a, hsa-miR-146a-5p, hsa-miR-1273f, hsa-miR-143-3p, hsa-miR-147a, hsa-miR-3622a-5p, hsa-miR-3911, hsa-miR-4269, hsa-miR-4708-5p, hsa-miR-4710, hsa-miR-4770, hsa-miR-4792, hsa-miR-574-5p, hsa-miR-6088, hsa-miR-644a, hsa-miR-6715b-5p, hsa-miR-6768-5p, hsa-miR-6867-5p, hsa-miR-7855-5p, hsa-miR-8485, and any combination thereof, may be administered to a glioblastoma subject to treat, ameliorate, and/or reduce the severity of glioblastoma or one or more symptoms thereof in the glioblastoma subject: (i) via a route selected from the group consisting of buccally, inhalation, intraarterially, intraarticularly, intracardially, intracerebroventricularly, intradermally, intramedullary, intramuscularly, intraperitoneally, intrathecally, intravenously, intraventricularly, nasally, ophthalmically, orally, rectally, sublabially, sublingually, subcutaneously, topically, and transdermally; and/or (ii) with a frequency selected from the group consisting of four times daily, three times daily, twice daily, once daily, once every two days, once every three days, once every four days, once every five days, once every six days, once a week, once every eight days, once every nine days, once every ten days, once every two weeks, once every three weeks, and once a month; and/or (iii) at a dosage (based on subject weight) selected from the group consisting of 0.1 μg/kg, 0.5 μg/kg, 1 μg/kg, 1.5 μg/kg, 2.0 μg/kg, 2.5 μg/kg, 3.0 μg/kg, 3.5 μg/kg, 4.0 μg/kg, 4.5 μg/kg, 5.0 μg/kg, 10 μg/kg, 15 μg/kg, 20 μg/kg, 25 μg/kg, 30 μg/kg, 35 μg/kg, 40 μg/kg, 45 μg/kg, 50 μg/kg, 55 μg/kg, 60 μg/kg, 65 μg/kg, 70 μg/kg, 75 μg/kg, 80 μg/kg, 85 μg/kg, 90 μg/kg, 95 μg/kg, 100 μg/kg, 150 μg/kg, 200 μg/kg, 250 μg/kg, 300 μg/kg, 350 μg/kg, 400 μg/kg, 450 μg/kg, 500 μg/kg, 550 μg/kg, 600 μg/kg, 650 μg/kg, 700 μg/kg, 750 μg/kg, 800 μg/kg, 850 μg/kg, 900 μg/kg, 1 mg/kg, 1.5 mg/kg, 2.0 mg/kg, 2.5 mg/kg, 3 mg/kg, 3.5 mg/kg, 4.0 mg/kg, 4.5 mg/kg, 5 mg/kg, 5.5 mg/kg, 6 mg/kg, 6.5 mg/kg, 7 mg/kg, 7.5 mg/kg, 8 mg/kg, 8.5 mg/kg, 9 mg/kg, 9.5 mg/kg, 10 mg/kg 10.5 mg/kg, 11 mg/kg, 11.5 mg/kg, 12 mg/kg, 12.5 mg/kg, 13 mg/kg, 13.5 mg/kg, 14 mg/kg, 14.5 mg/kg, 15 mg/kg, 16 mg/kg, 17 mg/kg, 18 mg/kg, 19 mg/kg, 20 mg/kg, 21 mg/kg, 22 mg/kg, 23 mg/kg, 24 mg/kg, 25 mg/kg, 30 mg/kg, 35 mg/kg, 40 mg/kg, 45 mg/kg, 50 mg/kg, 55 mg/kg, 60 mg/kg, 65 mg/kg, 70 mg/kg, 75 mg/kg, 80 mg/kg, 85 mg/kg, 90 mg/kg, 95 mg/kg, 100 mg/kg, 105 mg/kg, 110 mg/kg, 115 mg/kg, 120 mg/kg, 125 mg/kg, 130 mg/kg, 135 mg/kg, 140 mg/kg, 145 mg/kg, 150 mg/kg, 155 mg/kg, 160 mg/kg, 165 mg/kg, 170 mg/kg, 175 mg/kg, 180 mg/kg, 185 mg/kg, 190 mg/kg, 195 mg/kg, 200 mg/kg, and any fraction or integer in between any two of the preceding amounts.

In some embodiments, an nNOS inhibitor composition comprising one or more CRISPR/Cas systems, may be administered as a suitable dosage form, as described herein, to treat, ameliorate, and/or reduce the severity of glioblastoma or one or more symptoms thereof in a glioblastoma subject. In certain embodiments, an nNOS inhibitor composition comprising one or more CRISPR/Cas systems comprising an endonuclease selected from the group consisting of Cas1, Cas2, Cas3, Cas4, Cas5, Cas6, Cas7, Cas8a, Cas8b, Cas8c, Cas9, Cas10, Cas10d, Cas12, Cas12a, Cas12b, Cas12c, Cas12d, Cas12e, Cas12f, Cas12g, Cas12h, Cas12i, Cas12k, C2c4, C2c8, C2c9, Cas13, Cas13a, Cas13b, Cas13c, Cas13d, Cas13x.1, Cse1, Cse2, Csy1, Csy2, Csy3, GSU0054, Csm2, Cmr5, Csx10, Csx11, Csf1, and Csn2, may be administered as a suitable dosage form, as described herein, to treat, ameliorate, and/or reduce the severity of glioblastoma or one or more symptoms thereof in a glioblastoma subject. In some embodiments, an nNOS inhibitor composition comprising one or more CRISPR/Cas systems comprising an endonuclease selected from the group consisting of Cas1, Cas2, Cas3, Cas4, Cas5, Cas6, Cas7, Cas8a, Cas8b, Cas8c, Cas9, Cas10, Cas10d, Cas12, Cas12a, Cas12b, Cas12c, Cas12d, Cas12e, Cas12f, Cas12g, Cas12h, Cas12i, Cas12k, C2c4, C2c8, C2c9, Cas13, Cas13a, Cas13b, Cas13c, Cas13d, Cas13x.1, Cse1, Cse2, Csy1, Csy2, Csy3, GSU0054, Csm2, Cmr5, Csx10, Csx11, Csf1, and Csn2, may be administered to a glioblastoma subject to treat, ameliorate, and/or reduce the severity of glioblastoma or one or more symptoms thereof in the glioblastoma subject: (i) via a route selected from the group consisting of buccally, inhalation, intraarterially, intraarticularly, intracardially, intracerebroventricularly, intradermally, intramedullary, intramuscularly, intraperitoneally, intrathecally, intravenously, intraventricularly, nasally, ophthalmically, orally, rectally, sublabially, sublingually, subcutaneously, topically, and transdermally; and/or (ii) with a frequency selected from the group consisting of four times daily, three times daily, twice daily, once daily, once every two days, once every three days, once every four days, once every five days, once every six days, once a week, once every eight days, once every nine days, once every ten days, once every two weeks, once every three weeks, and once a month; and/or (iii) at a dosage (based on subject weight) selected from the group consisting of 0.1 μg/kg, 0.5 μg/kg, 1 μg/kg, 1.5 μg/kg, 2.0 μg/kg, 2.5 μg/kg, 3.0 μg/kg, 3.5 μg/kg, 4.0 μg/kg, 4.5 μg/kg, 5.0 μg/kg, 10 μg/kg, 15 μg/kg, 20 μg/kg, 25 μg/kg, 30 μg/kg, 35 μg/kg, 40 μg/kg, 45 μg/kg, 50 μg/kg, 55 μg/kg, 60 μg/kg, 65 μg/kg, 70 μg/kg, 75 μg/kg, 80 μg/kg, 85 μg/kg, 90 μg/kg, 95 μg/kg, 100 μg/kg, 150 μg/kg, 200 μg/kg, 250 μg/kg, 300 μg/kg, 350 μg/kg, 400 μg/kg, 450 μg/kg, 500 μg/kg, 550 μg/kg, 600 μg/kg, 650 μg/kg, 700 μg/kg, 750 μg/kg, 800 μg/kg, 850 μg/kg, 900 μg/kg, 1 mg/kg, 1.5 mg/kg, 2.0 mg/kg, 2.5 mg/kg, 3 mg/kg, 3.5 mg/kg, 4.0 mg/kg, 4.5 mg/kg, 5 mg/kg, 5.5 mg/kg, 6 mg/kg, 6.5 mg/kg, 7 mg/kg, 7.5 mg/kg, 8 mg/kg, 8.5 mg/kg, 9 mg/kg, 9.5 mg/kg, 10 mg/kg 10.5 mg/kg, 11 mg/kg, 11.5 mg/kg, 12 mg/kg, 12.5 mg/kg, 13 mg/kg, 13.5 mg/kg, 14 mg/kg, 14.5 mg/kg, 15 mg/kg, 16 mg/kg, 17 mg/kg, 18 mg/kg, 19 mg/kg, 20 mg/kg, 21 mg/kg, 22 mg/kg, 23 mg/kg, 24 mg/kg, 25 mg/kg, 30 mg/kg, 35 mg/kg, 40 mg/kg, 45 mg/kg, 50 mg/kg, 55 mg/kg, 60 mg/kg, 65 mg/kg, 70 mg/kg, 75 mg/kg, 80 mg/kg, 85 mg/kg, 90 mg/kg, 95 mg/kg, 100 mg/kg, 105 mg/kg, 110 mg/kg, 115 mg/kg, 120 mg/kg, 125 mg/kg, 130 mg/kg, 135 mg/kg, 140 mg/kg, 145 mg/kg, 150 mg/kg, 155 mg/kg, 160 mg/kg, 165 mg/kg, 170 mg/kg, 175 mg/kg, 180 mg/kg, 185 mg/kg, 190 mg/kg, 195 mg/kg, 200 mg/kg, and any fraction or integer in between any two of the preceding amounts.

In some embodiments, an nNOS inhibitor composition comprising one or more antibodies, may be administered as a suitable dosage form, as described herein, to treat, ameliorate, and/or reduce the severity of glioblastoma or one or more symptoms thereof in a glioblastoma subject. In certain embodiments, an nNOS inhibitor composition comprising one or more antibodies selected from the group consisting of 37-2800 (from ThermoFisher), EP1855Y (from abeam), EPR 24351-6 (from abeam), and any combination thereof, may be administered as a suitable dosage form, as described herein, to treat, ameliorate, and/or reduce the severity of glioblastoma or one or more symptoms thereof in a glioblastoma subject. In some embodiments, an nNOS inhibitor composition comprising one or more antibodies selected from the group consisting of 37-2800 (from ThermoFisher), EP1855Y (from abeam), EPR 24351-6 (from abeam), and any combination thereof, may be administered to a glioblastoma subject to treat, ameliorate, and/or reduce the severity of glioblastoma or one or more symptoms thereof in the glioblastoma subject: (i) via a route selected from the group consisting of buccally, inhalation, intraarterially, intraarticularly, intracardially, intracerebroventricularly, intradermally, intramedullary, intramuscularly, intraperitoneally, intrathecally, intravenously, intraventricularly, nasally, ophthalmically, orally, rectally, sublabially, sublingually, subcutaneously, topically, and transdermally; and/or (ii) with a frequency selected from the group consisting of four times daily, three times daily, twice daily, once daily, once every two days, once every three days, once every four days, once every five days, once every six days, once a week, once every eight days, once every nine days, once every ten days, once every two weeks, once every three weeks, and once a month; and/or (iii) at a dosage (based on subject weight) selected from the group consisting of 0.1 μg/kg, 0.5 μg/kg, 1 μg/kg, 1.5 μg/kg, 2.0 μg/kg, 2.5 μg/kg, 3.0 μg/kg, 3.5 μg/kg, 4.0 μg/kg, 4.5 μg/kg, 5.0 μg/kg, 10 μg/kg, 15 μg/kg, 20 μg/kg, 25 μg/kg, 30 μg/kg, 35 μg/kg, 40 μg/kg, 45 μg/kg, 50 μg/kg, 55 μg/kg, 60 μg/kg, 65 μg/kg, 70 μg/kg, 75 μg/kg, 80 μg/kg, 85 μg/kg, 90 μg/kg, 95 μg/kg, 100 μg/kg, 150 μg/kg, 200 μg/kg, 250 μg/kg, 300 μg/kg, 350 μg/kg, 400 μg/kg, 450 μg/kg, 500 μg/kg, 550 μg/kg, 600 μg/kg, 650 μg/kg, 700 μg/kg, 750 μg/kg, 800 μg/kg, 850 μg/kg, 900 μg/kg, 1 mg/kg, 1.5 mg/kg, 2.0 mg/kg, 2.5 mg/kg, 3 mg/kg, 3.5 mg/kg, 4.0 mg/kg, 4.5 mg/kg, 5 mg/kg, 5.5 mg/kg, 6 mg/kg, 6.5 mg/kg, 7 mg/kg, 7.5 mg/kg, 8 mg/kg, 8.5 mg/kg, 9 mg/kg, 9.5 mg/kg, 10 mg/kg 10.5 mg/kg, 11 mg/kg, 11.5 mg/kg, 12 mg/kg, 12.5 mg/kg, 13 mg/kg, 13.5 mg/kg, 14 mg/kg, 14.5 mg/kg, 15 mg/kg, 16 mg/kg, 17 mg/kg, 18 mg/kg, 19 mg/kg, 20 mg/kg, 21 mg/kg, 22 mg/kg, 23 mg/kg, 24 mg/kg, 25 mg/kg, 30 mg/kg, 35 mg/kg, 40 mg/kg, 45 mg/kg, 50 mg/kg, 55 mg/kg, 60 mg/kg, 65 mg/kg, 70 mg/kg, 75 mg/kg, 80 mg/kg, 85 mg/kg, 90 mg/kg, 95 mg/kg, 100 mg/kg, 105 mg/kg, 110 mg/kg, 115 mg/kg, 120 mg/kg, 125 mg/kg, 130 mg/kg, 135 mg/kg, 140 mg/kg, 145 mg/kg, 150 mg/kg, 155 mg/kg, 160 mg/kg, 165 mg/kg, 170 mg/kg, 175 mg/kg, 180 mg/kg, 185 mg/kg, 190 mg/kg, 195 mg/kg, 200 mg/kg, and any fraction or integer in between any two of the preceding amounts.

In some embodiments, an nNOS inhibitor composition comprising one or more amino acid-based compounds, may be administered as a suitable dosage form, as described herein, to treat, ameliorate, and/or reduce the severity of glioblastoma or one or more symptoms thereof in a glioblastoma subject. In certain embodiments, an nNOS inhibitor composition comprising one or more amino acid-compounds that bind an active site of nNOS selected from the group consisting of Phenylalanine 589, Tryptophan 592, Tyrosine 593, Glutamate 597, Aspartate 602, Arginine 601, Methionine 341, Histidine 342, and Tyrosine711, may be administered as a suitable dosage form, as described herein, to treat, ameliorate, and/or reduce the severity of glioblastoma or one or more symptoms thereof in a glioblastoma subject. In some embodiments, an nNOS inhibitor composition comprising one or more amino acid-compounds that bind an active site of nNOS selected from the group consisting of Phenylalanine 589, Tryptophan 592, Tyrosine 593, Glutamate 597, Aspartate 602, Arginine 601, Methionine 341, Histidine 342, and Tyrosine711, may be administered to a glioblastoma subject to treat, ameliorate, and/or reduce the severity of glioblastoma or one or more symptoms thereof in the glioblastoma subject: (i) via a route selected from the group consisting of buccally, inhalation, intraarterially, intraarticularly, intracardially, intracerebroventricularly, intradermally, intramedullary, intramuscularly, intraperitoneally, intrathecally, intravenously, intraventricularly, nasally, ophthalmically, orally, rectally, sublabially, sublingually, subcutaneously, topically, and transdermally; and/or (ii) with a frequency selected from the group consisting of four times daily, three times daily, twice daily, once daily, once every two days, once every three days, once every four days, once every five days, once every six days, once a week, once every eight days, once every nine days, once every ten days, once every two weeks, once every three weeks, and once a month; and/or (iii) at a dosage (based on subject weight) selected from the group consisting of 0.1 μg/kg, 0.5 μg/kg, 1 μg/kg, 1.5 μg/kg, 2.0 μg/kg, 2.5 μg/kg, 3.0 μg/kg, 3.5 μg/kg, 4.0 μg/kg, 4.5 μg/kg, 5.0 μg/kg, 10 μg/kg, 15 μg/kg, 20 μg/kg, 25 μg/kg, 30 μg/kg, 35 μg/kg, 40 μg/kg, 45 μg/kg, 50 μg/kg, 55 μg/kg, 60 μg/kg, 65 μg/kg, 70 μg/kg, 75 μg/kg, 80 μg/kg, 85 μg/kg, 90 μg/kg, 95 μg/kg, 100 μg/kg, 150 μg/kg, 200 μg/kg, 250 μg/kg, 300 μg/kg, 350 μg/kg, 400 μg/kg, 450 μg/kg, 500 μg/kg, 550 μg/kg, 600 μg/kg, 650 μg/kg, 700 μg/kg, 750 μg/kg, 800 μg/kg, 850 μg/kg, 900 μg/kg, 1 mg/kg, 1.5 mg/kg, 2.0 mg/kg, 2.5 mg/kg, 3 mg/kg, 3.5 mg/kg, 4.0 mg/kg, 4.5 mg/kg, 5 mg/kg, 5.5 mg/kg, 6 mg/kg, 6.5 mg/kg, 7 mg/kg, 7.5 mg/kg, 8 mg/kg, 8.5 mg/kg, 9 mg/kg, 9.5 mg/kg, 10 mg/kg 10.5 mg/kg, 11 mg/kg, 11.5 mg/kg, 12 mg/kg, 12.5 mg/kg, 13 mg/kg, 13.5 mg/kg, 14 mg/kg, 14.5 mg/kg, 15 mg/kg, 16 mg/kg, 17 mg/kg, 18 mg/kg, 19 mg/kg, 20 mg/kg, 21 mg/kg, 22 mg/kg, 23 mg/kg, 24 mg/kg, 25 mg/kg, 30 mg/kg, 35 mg/kg, 40 mg/kg, 45 mg/kg, 50 mg/kg, 55 mg/kg, 60 mg/kg, 65 mg/kg, 70 mg/kg, 75 mg/kg, 80 mg/kg, 85 mg/kg, 90 mg/kg, 95 mg/kg, 100 mg/kg, 105 mg/kg, 110 mg/kg, 115 mg/kg, 120 mg/kg, 125 mg/kg, 130 mg/kg, 135 mg/kg, 140 mg/kg, 145 mg/kg, 150 mg/kg, 155 mg/kg, 160 mg/kg, 165 mg/kg, 170 mg/kg, 175 mg/kg, 180 mg/kg, 185 mg/kg, 190 mg/kg, 195 mg/kg, 200 mg/kg, and any fraction or integer in between any two of the preceding amounts.

In some embodiments, a glioblastoma subject, as described herein, may be a female child or a male child. In certain embodiments, a glioblastoma subject, as described herein, may be a female adult or a male adult.

In some embodiments, a glioblastoma subject, as described herein, may have an abnormal expression of one or more antigens compared to an equivalent subject that does not have glioblastoma. As used herein, the term “equivalent subject,” refers to a subject of the same sex (male/female) and age range (child/adult) of the glioblastoma subject. For purposes of disclosure, the term “antigen,” when used with respect to abnormal expression, may define one or more cluster of differentiation (“CD”) antigens, such as, for example, CD15, CD44, CD70, CD133, as well as any CD antigen that has an abnormal expression in glioblastoma that would be readily envisaged by one skilled in the art. In certain embodiments, abnormal expression of an antigen may define an abnormal expression of CD15, an abnormal expression of CD44, an abnormal expression of CD70, an abnormal expression of CD133, an abnormal expression of a CD antigen that has an abnormal expression in glioblastoma that would be readily envisaged by one skilled in the art, and any combination thereof. In some embodiments, an “abnormal expression,” can refer to a reduced expression of an antigen, as described herein. In certain embodiments, an “abnormal expression,” can refer to an overexpression of an antigen, as described herein. Whether “abnormal expression,” refers to a reduced expression of an antigen or an overexpression of an antigen would be immediately envisaged by the skilled artisan when viewing the term in the context of the particular antigen disclosed and glioblastoma. For example, in the context of CD70 and glioblastoma, one skilled in the art would immediately envisage that “abnormal expression” refers to an overexpression of CD70. Treatment, amelioration, and/or reduction in the severity of glioblastoma or one or more symptoms thereof may be characterized by a restoration to a normal level of expression of one or more antigens from their abnormal state, as described herein. For example, in the context of CD70 and glioblastoma, treatment, amelioration, and/or reduction in the severity of glioblastoma or one or more symptoms thereof may be characterized by a reduced expression of CD70.

In certain embodiments, a glioblastoma subject, as described herein, may have an abnormal expression of one or more proteins compared to an equivalent subject that does not have glioblastoma. For purposes of disclosure, the term “protein,” when used with respect to abnormal expression, may define one or more proteins and/or enzymes, such as, for example, ALDH1A3, mTOR, p-mTOR, TSC-2, AKT, pAKT, RPS6, p-RPS6, Syp, VEGF, PIGF, as well as any protein or enzyme that has an abnormal expression in glioblastoma that would be readily envisaged by one skilled in the art. In certain embodiments, abnormal expression of a protein may define an abnormal expression of ALDH1A3, an abnormal expression of mTOR, an abnormal expression of p-mTOR, an abnormal expression of TSC-2, an abnormal expression of AKT, an abnormal expression of pAKT, an abnormal expression of RPS6, an abnormal expression of p-RPS6, an abnormal expression of Syp, an abnormal expression of VEGF, an abnormal expression of PIGF, an abnormal expression of a protein or enzyme that has an abnormal expression in glioblastoma that would be readily envisaged by one skilled in the art, and any combination thereof. In some embodiments, an “abnormal expression,” can refer to a reduced expression of a protein, as described herein. In certain embodiments, an “abnormal expression,” can refer to an overexpression of a protein, as described herein. Whether “abnormal expression,” refers to a reduced expression of a protein or an overexpression of a protein would be immediately envisaged by the skilled artisan when viewing the term in the context of the particular protein disclosed and glioblastoma. For example, in the context of glioblastoma, one skilled in the art would immediately envisage that “abnormal expression” refers to an overexpression of mTOR. Treatment, amelioration, and/or reduction in the severity of glioblastoma or one or more symptoms thereof may be characterized by a restoration to a normal level of expression of one or more proteins from their abnormal state, as described herein. For example, in the context of glioblastoma, treatment, amelioration, and/or reduction in the severity of glioblastoma or one or more symptoms thereof may be characterized by a reduced expression of mTOR.

In certain embodiments, a glioblastoma subject, as described herein, may have an increased expression of one or more NOS isoforms compared to an equivalent subject that does not have glioblastoma. For purposes of disclosure, the term “NOS isoform,” refers to nNOS, iNOS, and/or eNOS. In certain embodiments, a glioblastoma subject, as described herein, may have an increased expression of nNOS compared to an equivalent subject that does not have glioblastoma. In some embodiments, a glioblastoma subject, as described herein, may have an increased expression of iNOS compared to an equivalent subject that does not have glioblastoma. In certain embodiments, a glioblastoma subject, as described herein, may have an increased expression of eNOS compared to an equivalent subject that does not have glioblastoma. In some embodiments, a glioblastoma subject, as described herein, may have an increased expression of nNOS and iNOS compared to an equivalent subject that does not have glioblastoma. In certain embodiments, a glioblastoma subject, as described herein, may have an increased expression of nNOS and eNOS compared to an equivalent subject that does not have glioblastoma. In some embodiments, a glioblastoma subject, as described herein, may have an increased expression of iNOS and eNOS compared to an equivalent subject that does not have glioblastoma. In certain embodiments, a glioblastoma subject, as described herein, may have an increased expression of nNOS, iNOS, and eNOS compared to an equivalent subject that does not have glioblastoma. Whether a “NOS isoform,” has an increased expression would be immediately envisaged by the skilled artisan when viewing the term in the context of the particular NOS isoform disclosed and glioblastoma. For example, in the context of glioblastoma, one skilled in the art would immediately envisage that nNOS and/or iNOS have an increased expression. Treatment, amelioration, and/or reduction in the severity of glioblastoma or one or more symptoms thereof may be characterized by a reduced expression of one or more NOS isoforms from their increased state, as described herein. For example, in the context of glioblastoma, treatment, amelioration, and/or reduction in the severity of glioblastoma or one or more symptoms thereof may be characterized by a reduced expression of nNOS and/or iNOS. For purposes of disclosure, reduction/inhibition of the expression of one or more NOS isoforms may be characterized by a reduction in expression of one or more NOS isoforms by about at least 10%, about at least 20%, about at least 30%, or about at least 40%. In some embodiments, reduction/inhibition of the expression of one or more NOS isoforms may further be characterized by a lack reduction of other, non-implicated NOS isoforms, such as, for example, no more than about 10%, no more than about 5%, no more than about 2%, or no more than about 1% reduction/inhibition of the expression of non-implicated NOS isoforms. As used herein, the term “non-implicated NOS isoforms,” refers to NOS isoforms that do not have an increased expression in glioblastoma. By way of example, in the context of glioblastoma, if both nNOS and iNOS have an increased expression, eNOS would define a “non-implicated” NOS isoform. Whether a NOS isoform is a “non-implicated NOS isoform,” would be immediately envisaged by the skilled artisan when viewing the term in the context of the particular NOS isoform disclosed and glioblastoma.

In some embodiments, a glioblastoma subject, as described herein, may have an elevated level of one or more NO-indicators compared to an equivalent subject that does not have glioblastoma. As used herein, the term “NO-indicator,” can refer to 3-nitrotyrosine (3-Ntyr), NADPH-diaphorase, total nitrite concentration, total nitrate concentration, total nitrite and nitrate concentration, as well as any NO-indicator that has an elevated level in glioblastoma that would be readily envisaged by one skilled in the art. For example, in the context of glioblastoma, one skilled in the art would immediately envisage that 3-Ntyr and/or total nitrite and nitrate concentration can be present at an elevated level. Treatment, amelioration, and/or reduction in the severity of glioblastoma or one or more symptoms thereof may be characterized by a reduction in the level of one or more NO-indicators. For example, in the context of glioblastoma, treatment, amelioration, and/or reduction in the severity of glioblastoma or one or more symptoms thereof may be characterized by a reduction in the level of 3-Ntyr and/or total nitrite and nitrate concentration.

In certain embodiments, a NOS inhibitor composition, as described herein, may be administered as a suitable dosage form, as described herein, to treat, ameliorate, and/or reduce the severity of glioblastoma or one or more symptoms thereof, as an alternative to a conventional therapy for treating, ameliorating, and/or reducing the severity of glioblastoma or one or more symptoms thereof. For purposes of disclosure, a “conventional therapy,” as described herein, may refer to a treatment that would be recommended by one skilled in the art to treat glioblastoma, and may define one or more of radiation, chemotherapy, immunotherapy, cell-based therapy, gene therapy, and the like—one skilled in the art would immediately envisage a “conventional therapy” based on the particular glioblastoma disclosed, as well as characteristics of a glioblastoma subject, such as, for example, the subject's sex, age, stage of the glioblastoma, genotype of the glioblastoma, and the like. Treatment, amelioration, and/or reduction in the severity of glioblastoma or one or more symptoms thereof may be characterized by an improved outcome compared to a conventional therapy, as described herein. For purposes of the disclosure, an “improved outcome” may define any one of the following: (i) a larger reduction in tumor volume and/or mass compared to a subject administered an equivalent dose of a conventional therapy; (ii) an improvement in the expected rate of survival of a subject, at, for example, 1 month, 3 months, 6 months, 9 months, 1 year, 2 years, 3 years, 5 years, 10 years, or more, compared to the expected rate of survival of a subject administered an equivalent dose of a conventional therapy; (iii) a reduction in the prevalence of side effects resulting from the NOS inhibitor composition compared to a subject administered an equivalent dose of a conventional therapy; (iv) a reduction in the severity of side effects resulting from the NOS inhibitor composition compared to a subject administered an equivalent dose of a conventional therapy; (v) a reduction in the dosage required to achieve an equivalent reduction in tumor volume compared to a subject administered a conventional therapy; (vi) a reduction in the dosage required to achieve an equivalent expected rate of survival of a subject compared to a subject administered a conventional therapy; (vii) a reduction in the likelihood of a recurrence of the glioblastoma occurring in a subject compared to a subject administered an equivalent dose of a conventional therapy; (viii) a reduction in the likelihood of a metastasis occurring in a subject compared to a subject administered an equivalent dose of a conventional therapy; (ix) a reduction in the time required to be at a medical facility for treatment compared to a subject administered an equivalent dose of a conventional therapy; (x) a reduction in the cost associated with treatment compared to a subject administered an equivalent dose of a conventional therapy; as well as any other improved outcome not expressly recited that would be readily envisaged by one skilled in the art.

In some embodiments, a NOS inhibitor composition, as described herein, may be administered as a suitable dosage form, as described herein, to treat, ameliorate, and/or reduce the severity of glioblastoma or one or more symptoms thereof, wherein the NOS inhibitor composition comprises a first chemical NOS inhibitor and a second chemical NOS inhibitor. In certain embodiments, the first chemical NOS inhibitor may be administered concurrently with the second chemical NOS inhibitor. In some embodiments, the first chemical NOS inhibitor may be administered prior to the second chemical NOS inhibitor. In certain embodiments, the first chemical NOS inhibitor may be administered subsequently to the second chemical NOS inhibitor. Treatment, amelioration, and/or reduction in the severity of glioblastoma or one or more symptoms thereof may be characterized by an improved outcome compared an alternative treatment. As used herein, the term “alternative treatment,” may define one or more of a conventional therapy, as described herein, a NOS inhibitor composition comprising a single chemical NOS inhibitor, as described herein, and a NOS inhibitor composition comprising a single biological NOS inhibitor, as described herein. For purposes of the disclosure, an “improved outcome” may define any one of the following: (i) a larger reduction in tumor volume and/or mass compared an alternative treatment; (ii) an improvement in the expected rate of survival of a subject, at, for example, 1 month, 3 months, 6 months, 9 months, 1 year, 2 years, 3 years, 5 years, 10 years, or more, compared to the expected rate of survival of a subject administered an alternative treatment; (iii) a reduction in the prevalence of side effects resulting from the NOS inhibitor composition comprising at least two chemical NOS inhibitors compared to a subject administered an alternative treatment; (iv) a reduction in the severity of side effects resulting from the NOS inhibitor composition comprising at least two chemical NOS inhibitors compared to a subject administered an alternative treatment; (v) a reduction in the dosage required to achieve an equivalent reduction in tumor volume compared to a subject administered an alternative treatment; (vi) a reduction in the dosage required to achieve an equivalent expected rate of survival of a subject compared to a subject administered an alternative treatment; (vii) a reduction in the likelihood of a recurrence of the glioblastoma in a subject compared to a subject administered an alternative treatment; (viii) a reduction in the likelihood of a metastasis occurring in a subject compared to a subject administered an alternative treatment; (ix) a reduction in the time required to be at a medical facility for treatment compared to a subject administered an alternative treatment; (x) a reduction in the cost associated with treatment compared to a subject administered an alternative treatment; as well as any other improved outcome not expressly recited that would be readily envisaged by one skilled in the art.

In certain embodiments, a NOS inhibitor composition, as described herein, may be used to treat, ameliorate, and/or reduce the severity of glioblastoma or one or more symptoms thereof, wherein the NOS inhibitor composition comprises at least one chemical NOS inhibitor and at least one biological NOS inhibitor. In some embodiments, the at least one chemical NOS inhibitor may be administered concurrently with the at least one biological NOS inhibitor. In certain embodiments, the at least one chemical NOS inhibitor may be administered prior to the at least one biological NOS inhibitor. In some embodiments, the at least one chemical NOS inhibitor may be administered subsequently to the at least one biological NOS inhibitor. Treatment, amelioration, and/or reduction in the severity of glioblastoma or one or more symptoms thereof may be characterized by an improved outcome compared an alternative treatment. As used herein, the term “alternative treatment,” may define one or more of a conventional therapy, as described herein, a NOS inhibitor composition comprising a single chemical NOS inhibitor, as described herein, and a NOS inhibitor composition comprising a single biological NOS inhibitor, as described herein. For purposes of the disclosure, an “improved outcome” may define any one of the following: (i) a larger reduction in tumor volume and/or mass compared an alternative treatment; (ii) an improvement in the expected rate of survival of a subject, at, for example, 1 month, 3 months, 6 months, 9 months, 1 year, 2 years, 3 years, 5 years, 10 years, or more, compared to the expected rate of survival of a subject administered an alternative treatment; (iii) a reduction in the prevalence of side effects resulting from the NOS inhibitor composition comprising at least one chemical NOS inhibitor and at least one biological NOS inhibitor compared to a subject administered an alternative treatment; (iv) a reduction in the severity of side effects resulting from the NOS inhibitor composition comprising at least one chemical NOS inhibitor and at least one biological NOS inhibitor compared to a subject administered an alternative treatment; (v) a reduction in the dosage required to achieve an equivalent reduction in tumor volume compared to a subject administered an alternative treatment; (vi) a reduction in the dosage required to achieve an equivalent expected rate of survival of a subject compared to a subject administered an alternative treatment; (vii) a reduction in the likelihood of a recurrence of the glioblastoma in a subject compared to a subject administered an alternative treatment; (viii) a reduction in the likelihood of a metastasis occurring in a subject compared to a subject administered an alternative treatment; (ix) a reduction in the time required to be at a medical facility for treatment compared to a subject administered an alternative treatment; (x) a reduction in the cost associated with treatment compared to a subject administered an alternative treatment; as well as any other improved outcome not expressly recited that would be readily envisaged by one skilled in the art.

In some embodiments, a NOS inhibitor composition, as described herein, may be combined with a conventional therapy, as described herein, to treat, ameliorate, and/or reduce the severity of glioblastoma or one or more symptoms thereof. In certain embodiments, the NOS inhibitor composition may be administered concurrently with the conventional therapy. In some embodiments, the NOS inhibitor composition may be administered prior to the conventional therapy. In certain embodiments, the NOS inhibitor composition may be administered subsequently to the conventional therapy. Treatment, amelioration, and/or reduction in the severity of glioblastoma or one or more symptoms thereof may be characterized by an improved outcome compared an alternative treatment. As used herein, the term “alternative treatment,” may define one or more of a conventional therapy, as described herein, a NOS inhibitor composition comprising a single chemical NOS inhibitor, as described herein, and a NOS inhibitor composition comprising a single biological NOS inhibitor, as described herein. For purposes of the disclosure, an “improved outcome” may define any one of the following: (i) a larger reduction in tumor volume and/or mass compared an alternative treatment; (ii) an improvement in the expected rate of survival of a subject, at, for example, 1 month, 3 months, 6 months, 9 months, 1 year, 2 years, 3 years, 5 years, 10 years, or more, compared to the expected rate of survival of a subject administered an alternative treatment; (iii) a reduction in the prevalence of side effects resulting from the NOS inhibitor composition and conventional therapy compared to a subject administered an alternative treatment; (iv) a reduction in the severity of side effects resulting from the NOS inhibitor composition and conventional therapy compared to a subject administered an alternative treatment; (v) a reduction in the dosage required to achieve an equivalent reduction in tumor volume compared to a subject administered an alternative treatment; (vi) a reduction in the dosage required to achieve an equivalent expected rate of survival of a subject compared to a subject administered an alternative treatment; (vii) a reduction in the likelihood of a recurrence of the glioblastoma in a subject compared to a subject administered an alternative treatment; (viii) a reduction in the likelihood of a metastasis occurring in a subject compared to a subject administered an alternative treatment; (ix) a reduction in the time required to be at a medical facility for treatment compared to a subject administered an alternative treatment; (x) a reduction in the cost associated with treatment compared to a subject administered an alternative treatment; as well as any other improved outcome not expressly recited that would be readily envisaged by one skilled in the art.

In some embodiments, a “conventional therapy,” as that term is used with respect to combined administration with a NOS inhibitor composition, may refer to a conventional chemotherapy. For purposes of the disclosure, a “conventional chemotherapy,” as described herein may be a compound selected from the group consisting of abemaciclib, abiraterone acetate, acalabrutinib, adagrasib, afatinib dimaleate, albumin-bound paclitaxel, albumin-bound rapamycin (Sirolimus Protein-Bound Particles), alectinib, alpelisib, amifostine, aminolevulinic acid hydrochloride, anastrozole, apalutamide, aprepitant, arsenic trioxide, asciminib hydrochloride, avapritinib, axitinib, azacytidine, belinostat, belzutifan, bendamustine hydrochloride, bexarotene, bicalutamide, binimetinib, bleomycin, bleomycin sulfate, bortezomib, bosutinib, brigatinib, busulfan, cabazitaxel, cabozantinib-S-malate, capecitabine, capmatinib hydrochloride, carboplatin, carfilzomib, carmustine, cedazuridine, ceritinib, chlorambucil, cisplatin, cladribine, clofarabine, cobimetinib fumarate, copanlisib hydrochloride, crizotinib, cyclophosphamide, cytarabine, dabrafenib mesylate, dacarbazine, dacomitinib, dactinomycin, darolutamide, dasatinib, daunorubicin hydrochloride, decitabine, defibrotide sodium, degarelix, dexamethasone, dexrazoxane hydrochloride, docetaxel, doxorubicin hydrochloride, duvelisib, elacestrant dihydrochloride, eltrombopag olamine, enasidenib mesylate, encorafenib, entrectinib, enzalutamide, epirubicin hydrochloride, erdafitinib, eribulin mesylate, erlotinib hydrochloride, etoposide, etoposide phosphate, everolimus, exemestane, fedratinib hydrochloride, fludarabine phosphate, fluorouracil, flutamide, folinic acid, fostamatinib disodium, fulvestrant, futibatinib, gefitinib, gemcitabine hydrochloride, gilteritinib fumarate, glasdegib maleate, goserelin acetate, granisetron, granisetron hydrochloride, hydroxyurea, ibrutinib, idarubicin hydrochloride, idelalisib, ifosfamide, imatinib mesylate, imiquimod, infigratinib phosphate, iobenguane I-131, irinotecan hydrochloride, ivosidenib, ixabepilone, ixazomib citrate, lanreotide acetate, lapatinib ditosylate, larotrectinib sulfate, lenalidomide, lenvatinib mesylate, letrozole, leucovorin calcium, leuprolide acetate, liposomal cytarabine, liposomal doxorubicin hydrochloride, liposomal irinotecan hydrochloride, lomustine, lorlatinib, lurbinectedin, Lu-177 dotatate, Lu-177 vipivotide tetraxetan, mechlorethamine hydrochloride, megestrol acetate, melphalan, melphalan hydrochloride, mercaptopurine, mesna, methotrexate, methotrexate sodium, methylnaltrexone bromide, midostaurin, mitomycin, mitoxantrone hydrochloride, mobocertinib succinate, nelarabine, neratinib maleate, netupitant hydrochloride, nilutamide, nilotinib, niraparib tosylate monohydrate, olaparib, olutasidenib, omacetaxine mepesuccinate, ondansetron hydrochloride, osimertinib mesylate, oxaliplatin, paclitaxel, pacritinib citrate, palbociclib, palonosetron hydrochloride, pamidronate disodium, pazopanib hydrochloride, pemetrexed disodium, pemigatinib, pexidartinib hydrochloride, pirtobrutinib, plerixafor, pomalidomide, ponatinib hydrochloride, pralatrexate, pralsetinib, prednisone, procarbazine hydrochloride, propranolol hydrochloride, Ra-223 dichloride, raloxifene hydrochloride, regorafenib, relugolix, ribociclib, ripretinib, rolapitant hydrochloride, romidepsin, rucaparib camsylate, ruxolitinib phosphate, selinexor, selpercatinib, selumetinib sulfate, sonidegib, sorafenib tosylate, sotorasib, sunitinib malate, talazoparib tosylate, talc, tamoxifen citrate, tazemetostat hydrobromide, temozolomide, temsirolimus, tepotinib hydrochloride, thalidomide, thioguanine, thiotepa, tipiracil hydrochloride, tivozanib hydrochloride, topotecan hydrochloride, toremifene, trabectedin, trametinib dimethyl sulfoxide, tretinoin, trifluridine hydrochloride, tucatinib, uridine triacetate, valrubicin, vandetanib, vemurafenib, venetoclax, vinblastine sulfate, vincristine sulfate, vinorelbine tartrate, vismodegib, vorinostat, zanubrutinib, zoledronic acid, and any combination thereof. One skilled in the art would immediately envisage an appropriate “conventional chemotherapy” based on the particular glioblastoma disclosed, as well as characteristics of a glioblastoma subject, such as, for example, the subject's sex, age, stage of the glioblastoma, genotype of the glioblastoma, and the like.

In certain embodiments, a “conventional therapy,” as that term is used with respect to combined administration with a NOS inhibitor composition, may refer to a conventional immunotherapy. For purposes of the disclosure, a “conventional immunotherapy,” as described herein may be an immunotherapy, cell-based therapy, and/or gene-based therapy selected from the group consisting of ado-trastuzumab emtansine, aldesleukin, alemtuzumab, amivantamab-vmjw, asparaginase Erwinia chrysanthemi, asparaginase Erwinia chrysanthemi (recombinant)-rywn, atezolizumab, avelumab, axicabtagene ciloleucel, bevacizumab, blinatumomab, brentuximab vedotin, brexucabtagene autoleucel, calaspargase pegol-mknl, camrelizumab, caplacizumab-yhdp, cemiplimab-rwlc, cetuximab, ciltacabtagene autoleucel, daratumumab, daratumumab and hyaluronidase-fihj, darbepoetin alfa, denileukin diftitox, denosumab, dinutuximab, dostarlimab-gxly, durvalumab, elotuzumab, emapalumab-lzsg, enfortumab vedotin-ejfv, epoetin alfa, fam-trastuzumab deruxtecan-nxki, filgrastim, gemtuzumab ozogamicin, glucarpidase, ibritumomab tiuxetan, idecabtagene vicleucel, inotuzumab ozogamicin, ipilimumab, isatuximab-irfc, lisocabtagene maraleucel, loncastuximab tesirine-lpyl, luspatercept-aamt, margetuximab-cmkb, mirvetuximab soravtansine-gynx, mogamulizumab-kpkc, mosunetuzumab-axgb, moxetumomab pasudotox-tdfk, nadofaragene firadenovec-vncg, naxitamab-gqgk, necitumumab, nivolumab, nivolumab and relatlimab-rmbw, obinutuzumab, ofatumumab, palifermin, panitumumab, pegaspargase, pegfilgrastim, pembrolizumab, pertuzumab, pertuzumab, trastuzumab, and hyaluronidase-zzxf, polatuzumab vedotin-piiq, ramucirumab, rasburicase, ravulizumab-cwvz, recombinant HPV bivalent vaccine, recombinant HPV nonavalent vaccine, recombinant HPV quadrivalent vaccine, recombinant interferon alfa-2b, rituximab, rituximab and hyaluronidase human, romiplostim, ropeginterferon alfa-2b-njft, sacituzumab govitecan-hziy, siltuximab, sintilimab, sipuleucel-T, tafasitamab-cxix, tagraxofusp-erzs, talimogene laherparepvec, tebentafusp-tebn, teclistamab-cqyv, tisagenlecleucel, tislelizumab, tisotumab vedotin-tftv, tocilizumab, toripalimab, trastuzumab, trastuzumab and hyaluronidase-oysk, tremelimumab-actl, ziv-aflibercept, and any combination thereof. One skilled in the art would immediately envisage an appropriate “conventional immunotherapy” based on the particular glioblastoma disclosed, as well as characteristics of a glioblastoma subject, such as, for example, the subject's sex, age, stage of the glioblastoma, genotype of the glioblastoma, and the like.

In certain embodiments, a NOS inhibitor composition, as described herein, may be administered as a suitable dosage form, as described herein, prior to, or alternatively, following a surgery to remove or resect glioblastoma.

In some embodiments, a NOS inhibitor composition, as described herein, may be administered as a suitable dosage form, as described herein, to restore and/or maintain healthy and/or optimal expression levels of one or more antigens, as described herein, such as, for example, CD15, CD44, CD70, CD133, as well as any CD antigen that has an abnormal expression in glioblastoma that would be readily envisaged by one skilled in the art, in a subject. In certain embodiments, the subject may have glioblastoma or one or more symptoms thereof. As used herein, the terms “healthy” and “optimal” refer to expression levels of one or more antigens that would be typical of an equivalent subject that does not have glioblastoma.

In certain embodiments, a NOS inhibitor composition, as described herein, may be administered as a suitable dosage form, as described herein, to restore and/or maintain healthy and/or optimal expression levels of one or more proteins, as described herein, such as, for example, ALDH1A3, mTOR, p-mTOR, TSC-2, AKT, pAKT, RPS6, p-RPS6, Syp, VEGF, PIGF, as well as any protein or enzyme that has an abnormal expression in glioblastoma that would be readily envisaged by one skilled in the art, in a subject. In certain embodiments, the subject may have glioblastoma or one or more symptoms thereof. As used herein, the terms “healthy” and “optimal” refer to expression levels of one or more proteins that would be typical of an equivalent subject that does not have glioblastoma.

In some embodiments, a NOS inhibitor composition, as described herein, may be administered as a suitable dosage form, as described herein, to restore and/or maintain healthy and/or optimal expression levels of one or more NOS isoforms, as described herein, such as, for example, nNOS, iNOS, and eNOS, in a subject. In certain embodiments, the subject may have glioblastoma or one or more symptoms thereof. As used herein, the terms “healthy” and “optimal” refer to expression levels of one or more NOS isoforms that would be typical of an equivalent subject that does not have glioblastoma.

In certain embodiments, a NOS inhibitor composition, as described herein, may be administered as a suitable dosage form, as described herein, to restore and/or maintain healthy and/or optimal levels of one or more NO-indicators, as described herein, such as, for example, 3-Ntyr, NADPH-diaphorase, total nitrite concentration, total nitrate concentration, total nitrite and nitrate concentration, as well as any NO-indicator that has an elevated level in glioblastoma that would be readily envisaged by one skilled in the art, in a subject. In certain embodiments, the subject may have glioblastoma or one or more symptoms thereof. As used herein, the terms “healthy” and “optimal” refer to levels of one or more NO-indicators that would be typical of an equivalent subject that does not have glioblastoma.

In certain embodiments, a NOS inhibitor composition, as described herein, may be administered as a suitable dosage form, as described herein, to treat, ameliorate, and/or reduce the severity of neuroblastoma or one or more symptoms thereof in a subject (“neuroblastoma subject”).

In some embodiments, a NOS inhibitor composition comprising 7-NI and/or a salt thereof may be administered as a suitable dosage form, as described herein, to treat, ameliorate, and/or reduce the severity of neuroblastoma or one or more symptoms thereof in a neuroblastoma subject. In certain embodiments, an nNOS inhibitor composition comprising 7-NI and/or a salt thereof may be administered to a neuroblastoma subject to treat, ameliorate, and/or reduce the severity of neuroblastoma or one or more symptoms thereof in the neuroblastoma subject: (i) via a route selected from the group consisting of buccally, inhalation, intraarterially, intraarticularly, intracardially, intracerebroventricularly, intradermally, intramedullary, intramuscularly, intraperitoneally, intrathecally, intravenously, intraventricularly, nasally, ophthalmically, orally, rectally, sublabially, sublingually, subcutaneously, topically, and transdermally; and/or (ii) with a frequency selected from the group consisting of four times daily, three times daily, twice daily, once daily, once every two days, once every three days, once every four days, once every five days, once every six days, once a week, once every eight days, once every nine days, once every ten days, once every two weeks, once every three weeks, and once a month; and/or (iii) at a dosage (based on subject weight) selected from the group consisting of 0.1 μg/kg, 0.5 μg/kg, 1 μg/kg, 1.5 μg/kg, 2.0 μg/kg, 2.5 μg/kg, 3.0 μg/kg, 3.5 μg/kg, 4.0 μg/kg, 4.5 μg/kg, 5.0 μg/kg, 10 μg/kg, 15 μg/kg, 20 μg/kg, 25 μg/kg, 30 μg/kg, 35 μg/kg, 40 μg/kg, 45 μg/kg, 50 μg/kg, 55 μg/kg, 60 μg/kg, 65 μg/kg, 70 μg/kg, 75 μg/kg, 80 μg/kg, 85 μg/kg, 90 μg/kg, 95 μg/kg, 100 μg/kg, 150 μg/kg, 200 μg/kg, 250 μg/kg, 300 μg/kg, 350 μg/kg, 400 μg/kg, 450 μg/kg, 500 μg/kg, 550 μg/kg, 600 μg/kg, 650 μg/kg, 700 μg/kg, 750 μg/kg, 800 μg/kg, 850 μg/kg, 900 μg/kg, 1 mg/kg, 1.5 mg/kg, 2.0 mg/kg, 2.5 mg/kg, 3 mg/kg, 3.5 mg/kg, 4.0 mg/kg, 4.5 mg/kg, 5 mg/kg, 5.5 mg/kg, 6 mg/kg, 6.5 mg/kg, 7 mg/kg, 7.5 mg/kg, 8 mg/kg, 8.5 mg/kg, 9 mg/kg, 9.5 mg/kg, 10 mg/kg 10.5 mg/kg, 11 mg/kg, 11.5 mg/kg, 12 mg/kg, 12.5 mg/kg, 13 mg/kg, 13.5 mg/kg, 14 mg/kg, 14.5 mg/kg, 15 mg/kg, 16 mg/kg, 17 mg/kg, 18 mg/kg, 19 mg/kg, 20 mg/kg, 21 mg/kg, 22 mg/kg, 23 mg/kg, 24 mg/kg, 25 mg/kg, 30 mg/kg, 35 mg/kg, 40 mg/kg, 45 mg/kg, 50 mg/kg, 55 mg/kg, 60 mg/kg, 65 mg/kg, 70 mg/kg, 75 mg/kg, 80 mg/kg, 85 mg/kg, 90 mg/kg, 95 mg/kg, 100 mg/kg, 105 mg/kg, 110 mg/kg, 115 mg/kg, 120 mg/kg, 125 mg/kg, 130 mg/kg, 135 mg/kg, 140 mg/kg, 145 mg/kg, 150 mg/kg, 155 mg/kg, 160 mg/kg, 165 mg/kg, 170 mg/kg, 175 mg/kg, 180 mg/kg, 185 mg/kg, 190 mg/kg, 195 mg/kg, 200 mg/kg, and any fraction or integer in between any two of the preceding amounts.

In certain embodiments, a NOS inhibitor composition comprising 3-Br-7-NI and/or a salt thereof may be administered as a suitable dosage form, as described herein, to treat, ameliorate, and/or reduce the severity of neuroblastoma or one or more symptoms thereof in a neuroblastoma subject. In some embodiments, an nNOS inhibitor composition comprising 3-Br-7-NI and/or a salt thereof may be administered to a neuroblastoma subject to treat, ameliorate, and/or reduce the severity of neuroblastoma or one or more symptoms thereof in the neuroblastoma subject: (i) via a route selected from the group consisting of buccally, inhalation, intraarterially, intraarticularly, intracardially, intracerebroventricularly, intradermally, intramedullary, intramuscularly, intraperitoneally, intrathecally, intravenously, intraventricularly, nasally, ophthalmically, orally, rectally, sublabially, sublingually, subcutaneously, topically, and transdermally; and/or (ii) with a frequency selected from the group consisting of four times daily, three times daily, twice daily, once daily, once every two days, once every three days, once every four days, once every five days, once every six days, once a week, once every eight days, once every nine days, once every ten days, once every two weeks, once every three weeks, and once a month; and/or (iii) at a dosage (based on subject weight) selected from the group consisting of 0.1 μg/kg, 0.5 μg/kg, 1 μg/kg, 1.5 μg/kg, 2.0 μg/kg, 2.5 μg/kg, 3.0 μg/kg, 3.5 μg/kg, 4.0 μg/kg, 4.5 μg/kg, 5.0 μg/kg, 10 μg/kg, 15 μg/kg, 20 μg/kg, 25 μg/kg, 30 μg/kg, 35 μg/kg, 40 μg/kg, 45 μg/kg, 50 μg/kg, 55 μg/kg, 60 μg/kg, 65 μg/kg, 70 μg/kg, 75 μg/kg, 80 μg/kg, 85 μg/kg, 90 μg/kg, 95 μg/kg, 100 μg/kg, 150 μg/kg, 200 μg/kg, 250 μg/kg, 300 μg/kg, 350 μg/kg, 400 μg/kg, 450 μg/kg, 500 μg/kg, 550 μg/kg, 600 μg/kg, 650 μg/kg, 700 μg/kg, 750 μg/kg, 800 μg/kg, 850 μg/kg, 900 μg/kg, 1 mg/kg, 1.5 mg/kg, 2.0 mg/kg, 2.5 mg/kg, 3 mg/kg, 3.5 mg/kg, 4.0 mg/kg, 4.5 mg/kg, 5 mg/kg, 5.5 mg/kg, 6 mg/kg, 6.5 mg/kg, 7 mg/kg, 7.5 mg/kg, 8 mg/kg, 8.5 mg/kg, 9 mg/kg, 9.5 mg/kg, 10 mg/kg 10.5 mg/kg, 11 mg/kg, 11.5 mg/kg, 12 mg/kg, 12.5 mg/kg, 13 mg/kg, 13.5 mg/kg, 14 mg/kg, 14.5 mg/kg, 15 mg/kg, 16 mg/kg, 17 mg/kg, 18 mg/kg, 19 mg/kg, 20 mg/kg, 21 mg/kg, 22 mg/kg, 23 mg/kg, 24 mg/kg, 25 mg/kg, 30 mg/kg, 35 mg/kg, 40 mg/kg, 45 mg/kg, 50 mg/kg, 55 mg/kg, 60 mg/kg, 65 mg/kg, 70 mg/kg, 75 mg/kg, 80 mg/kg, 85 mg/kg, 90 mg/kg, 95 mg/kg, 100 mg/kg, 105 mg/kg, 110 mg/kg, 115 mg/kg, 120 mg/kg, 125 mg/kg, 130 mg/kg, 135 mg/kg, 140 mg/kg, 145 mg/kg, 150 mg/kg, 155 mg/kg, 160 mg/kg, 165 mg/kg, 170 mg/kg, 175 mg/kg, 180 mg/kg, 185 mg/kg, 190 mg/kg, 195 mg/kg, 200 mg/kg, and any fraction or integer in between any two of the preceding amounts.

In some embodiments, a NOS inhibitor composition comprising L-NPA and/or a salt thereof, such as, for example, L-NPA hydrochloride, may be administered as a suitable dosage form, as described herein, to treat, ameliorate, and/or reduce the severity of neuroblastoma or one or more symptoms thereof in a neuroblastoma subject. In certain embodiments, an nNOS inhibitor composition comprising L-NPA and/or a salt thereof, such as, for example, L-NPA hydrochloride may be administered to a neuroblastoma subject to treat, ameliorate, and/or reduce the severity of neuroblastoma or one or more symptoms thereof in the neuroblastoma subject: (i) via a route selected from the group consisting of buccally, inhalation, intraarterially, intraarticularly, intracardially, intracerebroventricularly, intradermally, intramedullary, intramuscularly, intraperitoneally, intrathecally, intravenously, intraventricularly, nasally, ophthalmically, orally, rectally, sublabially, sublingually, subcutaneously, topically, and transdermally; and/or (ii) with a frequency selected from the group consisting of four times daily, three times daily, twice daily, once daily, once every two days, once every three days, once every four days, once every five days, once every six days, once a week, once every eight days, once every nine days, once every ten days, once every two weeks, once every three weeks, and once a month; and/or (iii) at a dosage (based on subject weight) selected from the group consisting of 0.1 μg/kg, 0.5 μg/kg, 1 μg/kg, 1.5 μg/kg, 2.0 μg/kg, 2.5 μg/kg, 3.0 μg/kg, 3.5 μg/kg, 4.0 μg/kg, 4.5 μg/kg, 5.0 μg/kg, 10 μg/kg, 15 μg/kg, 20 μg/kg, 25 μg/kg, 30 μg/kg, 35 μg/kg, 40 μg/kg, 45 μg/kg, 50 μg/kg, 55 μg/kg, 60 μg/kg, 65 μg/kg, 70 μg/kg, 75 μg/kg, 80 μg/kg, 85 μg/kg, 90 μg/kg, 95 μg/kg, 100 μg/kg, 150 μg/kg, 200 μg/kg, 250 μg/kg, 300 μg/kg, 350 μg/kg, 400 μg/kg, 450 μg/kg, 500 μg/kg, 550 μg/kg, 600 μg/kg, 650 μg/kg, 700 μg/kg, 750 μg/kg, 800 μg/kg, 850 μg/kg, 900 μg/kg, 1 mg/kg, 1.5 mg/kg, 2.0 mg/kg, 2.5 mg/kg, 3 mg/kg, 3.5 mg/kg, 4.0 mg/kg, 4.5 mg/kg, 5 mg/kg, 5.5 mg/kg, 6 mg/kg, 6.5 mg/kg, 7 mg/kg, 7.5 mg/kg, 8 mg/kg, 8.5 mg/kg, 9 mg/kg, 9.5 mg/kg, 10 mg/kg 10.5 mg/kg, 11 mg/kg, 11.5 mg/kg, 12 mg/kg, 12.5 mg/kg, 13 mg/kg, 13.5 mg/kg, 14 mg/kg, 14.5 mg/kg, 15 mg/kg, 16 mg/kg, 17 mg/kg, 18 mg/kg, 19 mg/kg, 20 mg/kg, 21 mg/kg, 22 mg/kg, 23 mg/kg, 24 mg/kg, 25 mg/kg, 30 mg/kg, 35 mg/kg, 40 mg/kg, 45 mg/kg, 50 mg/kg, 55 mg/kg, 60 mg/kg, 65 mg/kg, 70 mg/kg, 75 mg/kg, 80 mg/kg, 85 mg/kg, 90 mg/kg, 95 mg/kg, 100 mg/kg, 105 mg/kg, 110 mg/kg, 115 mg/kg, 120 mg/kg, 125 mg/kg, 130 mg/kg, 135 mg/kg, 140 mg/kg, 145 mg/kg, 150 mg/kg, 155 mg/kg, 160 mg/kg, 165 mg/kg, 170 mg/kg, 175 mg/kg, 180 mg/kg, 185 mg/kg, 190 mg/kg, 195 mg/kg, 200 mg/kg, and any fraction or integer in between any two of the preceding amounts.

In certain embodiments, a NOS inhibitor composition comprising L-NIO and/or a salt thereof, such as, for example, L-NIO dihydrochloride, may be administered as a suitable dosage form, as described herein, to treat, ameliorate, and/or reduce the severity of neuroblastoma or one or more symptoms thereof in a neuroblastoma subject. In some embodiments, an nNOS inhibitor composition comprising L-NIO and/or a salt thereof, such as, for example, L-NIO dihydrochloride, may be administered to a neuroblastoma subject to treat, ameliorate, and/or reduce the severity of neuroblastoma or one or more symptoms thereof in the neuroblastoma subject: (i) via a route selected from the group consisting of buccally, inhalation, intraarterially, intraarticularly, intracardially, intracerebroventricularly, intradermally, intramedullary, intramuscularly, intraperitoneally, intrathecally, intravenously, intraventricularly, nasally, ophthalmically, orally, rectally, sublabially, sublingually, subcutaneously, topically, and transdermally; and/or (ii) with a frequency selected from the group consisting of four times daily, three times daily, twice daily, once daily, once every two days, once every three days, once every four days, once every five days, once every six days, once a week, once every eight days, once every nine days, once every ten days, once every two weeks, once every three weeks, and once a month; and/or (iii) at a dosage (based on subject weight) selected from the group consisting of 0.1 μg/kg, 0.5 μg/kg, 1 μg/kg, 1.5 μg/kg, 2.0 μg/kg, 2.5 μg/kg, 3.0 μg/kg, 3.5 μg/kg, 4.0 μg/kg, 4.5 μg/kg, 5.0 μg/kg, 10 μg/kg, 15 μg/kg, 20 μg/kg, 25 μg/kg, 30 μg/kg, 35 μg/kg, 40 μg/kg, 45 μg/kg, 50 μg/kg, 55 μg/kg, 60 μg/kg, 65 μg/kg, 70 μg/kg, 75 μg/kg, 80 μg/kg, 85 μg/kg, 90 μg/kg, 95 μg/kg, 100 μg/kg, 150 μg/kg, 200 μg/kg, 250 μg/kg, 300 μg/kg, 350 μg/kg, 400 μg/kg, 450 μg/kg, 500 μg/kg, 550 μg/kg, 600 μg/kg, 650 μg/kg, 700 μg/kg, 750 μg/kg, 800 μg/kg, 850 μg/kg, 900 μg/kg, 1 mg/kg, 1.5 mg/kg, 2.0 mg/kg, 2.5 mg/kg, 3 mg/kg, 3.5 mg/kg, 4.0 mg/kg, 4.5 mg/kg, 5 mg/kg, 5.5 mg/kg, 6 mg/kg, 6.5 mg/kg, 7 mg/kg, 7.5 mg/kg, 8 mg/kg, 8.5 mg/kg, 9 mg/kg, 9.5 mg/kg, 10 mg/kg 10.5 mg/kg, 11 mg/kg, 11.5 mg/kg, 12 mg/kg, 12.5 mg/kg, 13 mg/kg, 13.5 mg/kg, 14 mg/kg, 14.5 mg/kg, 15 mg/kg, 16 mg/kg, 17 mg/kg, 18 mg/kg, 19 mg/kg, 20 mg/kg, 21 mg/kg, 22 mg/kg, 23 mg/kg, 24 mg/kg, 25 mg/kg, 30 mg/kg, 35 mg/kg, 40 mg/kg, 45 mg/kg, 50 mg/kg, 55 mg/kg, 60 mg/kg, 65 mg/kg, 70 mg/kg, 75 mg/kg, 80 mg/kg, 85 mg/kg, 90 mg/kg, 95 mg/kg, 100 mg/kg, 105 mg/kg, 110 mg/kg, 115 mg/kg, 120 mg/kg, 125 mg/kg, 130 mg/kg, 135 mg/kg, 140 mg/kg, 145 mg/kg, 150 mg/kg, 155 mg/kg, 160 mg/kg, 165 mg/kg, 170 mg/kg, 175 mg/kg, 180 mg/kg, 185 mg/kg, 190 mg/kg, 195 mg/kg, 200 mg/kg, and any fraction or integer in between any two of the preceding amounts.

In some embodiments, a NOS inhibitor composition comprising ARL-17477 and/or a salt thereof, such as, for example, ARL-17477 dihydrochloride, may be administered as a suitable dosage form, as described herein, to treat, ameliorate, and/or reduce the severity of neuroblastoma or one or more symptoms thereof in a neuroblastoma subject. In certain embodiments, an nNOS inhibitor composition comprising ARL-17477 and/or a salt thereof, such as, for example, ARL-17477 dihydrochloride, may be administered to a neuroblastoma subject to treat, ameliorate, and/or reduce the severity of neuroblastoma or one or more symptoms thereof in the neuroblastoma subject: (i) via a route selected from the group consisting of buccally, inhalation, intraarterially, intraarticularly, intracardially, intracerebroventricularly, intradermally, intramedullary, intramuscularly, intraperitoneally, intrathecally, intravenously, intraventricularly, nasally, ophthalmically, orally, rectally, sublabially, sublingually, subcutaneously, topically, and transdermally; and/or (ii) with a frequency selected from the group consisting of four times daily, three times daily, twice daily, once daily, once every two days, once every three days, once every four days, once every five days, once every six days, once a week, once every eight days, once every nine days, once every ten days, once every two weeks, once every three weeks, and once a month; and/or (iii) at a dosage (based on subject weight) selected from the group consisting of 0.1 μg/kg, 0.5 μg/kg, 1 μg/kg, 1.5 μg/kg, 2.0 μg/kg, 2.5 μg/kg, 3.0 μg/kg, 3.5 μg/kg, 4.0 μg/kg, 4.5 μg/kg, 5.0 μg/kg, 10 μg/kg, 15 μg/kg, 20 μg/kg, 25 μg/kg, 30 μg/kg, 35 μg/kg, 40 μg/kg, 45 μg/kg, 50 μg/kg, 55 μg/kg, 60 μg/kg, 65 μg/kg, 70 μg/kg, 75 μg/kg, 80 μg/kg, 85 μg/kg, 90 μg/kg, 95 μg/kg, 100 μg/kg, 150 μg/kg, 200 μg/kg, 250 μg/kg, 300 μg/kg, 350 μg/kg, 400 μg/kg, 450 μg/kg, 500 μg/kg, 550 μg/kg, 600 μg/kg, 650 μg/kg, 700 μg/kg, 750 μg/kg, 800 μg/kg, 850 μg/kg, 900 μg/kg, 1 mg/kg, 1.5 mg/kg, 2.0 mg/kg, 2.5 mg/kg, 3 mg/kg, 3.5 mg/kg, 4.0 mg/kg, 4.5 mg/kg, 5 mg/kg, 5.5 mg/kg, 6 mg/kg, 6.5 mg/kg, 7 mg/kg, 7.5 mg/kg, 8 mg/kg, 8.5 mg/kg, 9 mg/kg, 9.5 mg/kg, 10 mg/kg 10.5 mg/kg, 11 mg/kg, 11.5 mg/kg, 12 mg/kg, 12.5 mg/kg, 13 mg/kg, 13.5 mg/kg, 14 mg/kg, 14.5 mg/kg, 15 mg/kg, 16 mg/kg, 17 mg/kg, 18 mg/kg, 19 mg/kg, 20 mg/kg, 21 mg/kg, 22 mg/kg, 23 mg/kg, 24 mg/kg, 25 mg/kg, 30 mg/kg, 35 mg/kg, 40 mg/kg, 45 mg/kg, 50 mg/kg, 55 mg/kg, 60 mg/kg, 65 mg/kg, 70 mg/kg, 75 mg/kg, 80 mg/kg, 85 mg/kg, 90 mg/kg, 95 mg/kg, 100 mg/kg, 105 mg/kg, 110 mg/kg, 115 mg/kg, 120 mg/kg, 125 mg/kg, 130 mg/kg, 135 mg/kg, 140 mg/kg, 145 mg/kg, 150 mg/kg, 155 mg/kg, 160 mg/kg, 165 mg/kg, 170 mg/kg, 175 mg/kg, 180 mg/kg, 185 mg/kg, 190 mg/kg, 195 mg/kg, 200 mg/kg, and any fraction or integer in between any two of the preceding amounts.

In certain embodiments, a NOS inhibitor composition comprising L-NNA and/or a salt thereof may be administered as a suitable dosage form, as described herein, to treat, ameliorate, and/or reduce the severity of neuroblastoma or one or more symptoms thereof in a neuroblastoma subject. In some embodiments, an nNOS inhibitor composition comprising L-NNA and/or a salt thereof may be administered to a neuroblastoma subject to treat, ameliorate, and/or reduce the severity of neuroblastoma or one or more symptoms thereof in the neuroblastoma subject: (i) via a route selected from the group consisting of buccally, inhalation, intraarterially, intraarticularly, intracardially, intracerebroventricularly, intradermally, intramedullary, intramuscularly, intraperitoneally, intrathecally, intravenously, intraventricularly, nasally, ophthalmically, orally, rectally, sublabially, sublingually, subcutaneously, topically, and transdermally; and/or (ii) with a frequency selected from the group consisting of four times daily, three times daily, twice daily, once daily, once every two days, once every three days, once every four days, once every five days, once every six days, once a week, once every eight days, once every nine days, once every ten days, once every two weeks, once every three weeks, and once a month; and/or (iii) at a dosage (based on subject weight) selected from the group consisting of 0.1 μg/kg, 0.5 μg/kg, 1 μg/kg, 1.5 μg/kg, 2.0 μg/kg, 2.5 μg/kg, 3.0 μg/kg, 3.5 μg/kg, 4.0 μg/kg, 4.5 μg/kg, 5.0 μg/kg, 10 μg/kg, 15 μg/kg, 20 μg/kg, 25 μg/kg, 30 μg/kg, 35 μg/kg, 40 μg/kg, 45 μg/kg, 50 μg/kg, 55 μg/kg, 60 μg/kg, 65 μg/kg, 70 μg/kg, 75 μg/kg, 80 μg/kg, 85 μg/kg, 90 μg/kg, 95 μg/kg, 100 μg/kg, 150 μg/kg, 200 μg/kg, 250 μg/kg, 300 μg/kg, 350 μg/kg, 400 μg/kg, 450 μg/kg, 500 μg/kg, 550 μg/kg, 600 μg/kg, 650 μg/kg, 700 μg/kg, 750 μg/kg, 800 μg/kg, 850 μg/kg, 900 μg/kg, 1 mg/kg, 1.5 mg/kg, 2.0 mg/kg, 2.5 mg/kg, 3 mg/kg, 3.5 mg/kg, 4.0 mg/kg, 4.5 mg/kg, 5 mg/kg, 5.5 mg/kg, 6 mg/kg, 6.5 mg/kg, 7 mg/kg, 7.5 mg/kg, 8 mg/kg, 8.5 mg/kg, 9 mg/kg, 9.5 mg/kg, 10 mg/kg 10.5 mg/kg, 11 mg/kg, 11.5 mg/kg, 12 mg/kg, 12.5 mg/kg, 13 mg/kg, 13.5 mg/kg, 14 mg/kg, 14.5 mg/kg, 15 mg/kg, 16 mg/kg, 17 mg/kg, 18 mg/kg, 19 mg/kg, 20 mg/kg, 21 mg/kg, 22 mg/kg, 23 mg/kg, 24 mg/kg, 25 mg/kg, 30 mg/kg, 35 mg/kg, 40 mg/kg, 45 mg/kg, 50 mg/kg, 55 mg/kg, 60 mg/kg, 65 mg/kg, 70 mg/kg, 75 mg/kg, 80 mg/kg, 85 mg/kg, 90 mg/kg, 95 mg/kg, 100 mg/kg, 105 mg/kg, 110 mg/kg, 115 mg/kg, 120 mg/kg, 125 mg/kg, 130 mg/kg, 135 mg/kg, 140 mg/kg, 145 mg/kg, 150 mg/kg, 155 mg/kg, 160 mg/kg, 165 mg/kg, 170 mg/kg, 175 mg/kg, 180 mg/kg, 185 mg/kg, 190 mg/kg, 195 mg/kg, 200 mg/kg, and any fraction or integer in between any two of the preceding amounts.

In some embodiments, a NOS inhibitor composition comprising L-NAME and/or a salt thereof, such as, for example, L-NAME hydrochloride, may be administered as a suitable dosage form, as described herein, to treat, ameliorate, and/or reduce the severity of neuroblastoma or one or more symptoms thereof in a neuroblastoma subject. In certain embodiments, an nNOS inhibitor composition comprising L-NAME and/or a salt thereof, such as, for example, L-NAME hydrochloride, may be administered to a neuroblastoma subject to treat, ameliorate, and/or reduce the severity of neuroblastoma or one or more symptoms thereof in the neuroblastoma subject: (i) via a route selected from the group consisting of buccally, inhalation, intraarterially, intraarticularly, intracardially, intracerebroventricularly, intradermally, intramedullary, intramuscularly, intraperitoneally, intrathecally, intravenously, intraventricularly, nasally, ophthalmically, orally, rectally, sublabially, sublingually, subcutaneously, topically, and transdermally; and/or (ii) with a frequency selected from the group consisting of four times daily, three times daily, twice daily, once daily, once every two days, once every three days, once every four days, once every five days, once every six days, once a week, once every eight days, once every nine days, once every ten days, once every two weeks, once every three weeks, and once a month; and/or (iii) at a dosage (based on subject weight) selected from the group consisting of 0.1 μg/kg, 0.5 μg/kg, 1 μg/kg, 1.5 μg/kg, 2.0 μg/kg, 2.5 μg/kg, 3.0 μg/kg, 3.5 μg/kg, 4.0 μg/kg, 4.5 μg/kg, 5.0 μg/kg, 10 μg/kg, 15 μg/kg, 20 μg/kg, 25 μg/kg, 30 μg/kg, 35 μg/kg, 40 μg/kg, 45 μg/kg, 50 μg/kg, 55 μg/kg, 60 μg/kg, 65 μg/kg, 70 μg/kg, 75 μg/kg, 80 μg/kg, 85 μg/kg, 90 μg/kg, 95 μg/kg, 100 μg/kg, 150 μg/kg, 200 μg/kg, 250 μg/kg, 300 μg/kg, 350 μg/kg, 400 μg/kg, 450 μg/kg, 500 μg/kg, 550 μg/kg, 600 μg/kg, 650 μg/kg, 700 μg/kg, 750 μg/kg, 800 μg/kg, 850 μg/kg, 900 μg/kg, 1 mg/kg, 1.5 mg/kg, 2.0 mg/kg, 2.5 mg/kg, 3 mg/kg, 3.5 mg/kg, 4.0 mg/kg, 4.5 mg/kg, 5 mg/kg, 5.5 mg/kg, 6 mg/kg, 6.5 mg/kg, 7 mg/kg, 7.5 mg/kg, 8 mg/kg, 8.5 mg/kg, 9 mg/kg, 9.5 mg/kg, 10 mg/kg 10.5 mg/kg, 11 mg/kg, 11.5 mg/kg, 12 mg/kg, 12.5 mg/kg, 13 mg/kg, 13.5 mg/kg, 14 mg/kg, 14.5 mg/kg, 15 mg/kg, 16 mg/kg, 17 mg/kg, 18 mg/kg, 19 mg/kg, 20 mg/kg, 21 mg/kg, 22 mg/kg, 23 mg/kg, 24 mg/kg, 25 mg/kg, 30 mg/kg, 35 mg/kg, 40 mg/kg, 45 mg/kg, 50 mg/kg, 55 mg/kg, 60 mg/kg, 65 mg/kg, 70 mg/kg, 75 mg/kg, 80 mg/kg, 85 mg/kg, 90 mg/kg, 95 mg/kg, 100 mg/kg, 105 mg/kg, 110 mg/kg, 115 mg/kg, 120 mg/kg, 125 mg/kg, 130 mg/kg, 135 mg/kg, 140 mg/kg, 145 mg/kg, 150 mg/kg, 155 mg/kg, 160 mg/kg, 165 mg/kg, 170 mg/kg, 175 mg/kg, 180 mg/kg, 185 mg/kg, 190 mg/kg, 195 mg/kg, 200 mg/kg, and any fraction or integer in between any two of the preceding amounts.

In certain embodiments, a NOS inhibitor composition comprising L-NMMA and/or a salt thereof, such as, for example, L-NMMA acetate, may be administered as a suitable dosage form, as described herein, to treat, ameliorate, and/or reduce the severity of neuroblastoma or one or more symptoms thereof in a neuroblastoma subject. In some embodiments, an nNOS inhibitor composition comprising L-NMMA and/or a salt thereof, such as, for example, L-NMMA acetate, may be administered to a neuroblastoma subject to treat, ameliorate, and/or reduce the severity of neuroblastoma or one or more symptoms thereof in the neuroblastoma subject: (i) via a route selected from the group consisting of buccally, inhalation, intraarterially, intraarticularly, intracardially, intracerebroventricularly, intradermally, intramedullary, intramuscularly, intraperitoneally, intrathecally, intravenously, intraventricularly, nasally, ophthalmically, orally, rectally, sublabially, sublingually, subcutaneously, topically, and transdermally; and/or (ii) with a frequency selected from the group consisting of four times daily, three times daily, twice daily, once daily, once every two days, once every three days, once every four days, once every five days, once every six days, once a week, once every eight days, once every nine days, once every ten days, once every two weeks, once every three weeks, and once a month; and/or (iii) at a dosage (based on subject weight) selected from the group consisting of 0.1 μg/kg, 0.5 μg/kg, 1 μg/kg, 1.5 μg/kg, 2.0 μg/kg, 2.5 μg/kg, 3.0 μg/kg, 3.5 μg/kg, 4.0 μg/kg, 4.5 μg/kg, 5.0 μg/kg, 10 μg/kg, 15 μg/kg, 20 μg/kg, 25 μg/kg, 30 μg/kg, 35 μg/kg, 40 μg/kg, 45 μg/kg, 50 μg/kg, 55 μg/kg, 60 μg/kg, 65 μg/kg, 70 μg/kg, 75 μg/kg, 80 μg/kg, 85 μg/kg, 90 μg/kg, 95 μg/kg, 100 μg/kg, 150 μg/kg, 200 μg/kg, 250 μg/kg, 300 μg/kg, 350 μg/kg, 400 μg/kg, 450 μg/kg, 500 μg/kg, 550 μg/kg, 600 μg/kg, 650 μg/kg, 700 μg/kg, 750 μg/kg, 800 μg/kg, 850 μg/kg, 900 μg/kg, 1 mg/kg, 1.5 mg/kg, 2.0 mg/kg, 2.5 mg/kg, 3 mg/kg, 3.5 mg/kg, 4.0 mg/kg, 4.5 mg/kg, 5 mg/kg, 5.5 mg/kg, 6 mg/kg, 6.5 mg/kg, 7 mg/kg, 7.5 mg/kg, 8 mg/kg, 8.5 mg/kg, 9 mg/kg, 9.5 mg/kg, 10 mg/kg 10.5 mg/kg, 11 mg/kg, 11.5 mg/kg, 12 mg/kg, 12.5 mg/kg, 13 mg/kg, 13.5 mg/kg, 14 mg/kg, 14.5 mg/kg, 15 mg/kg, 16 mg/kg, 17 mg/kg, 18 mg/kg, 19 mg/kg, 20 mg/kg, 21 mg/kg, 22 mg/kg, 23 mg/kg, 24 mg/kg, 25 mg/kg, 30 mg/kg, 35 mg/kg, 40 mg/kg, 45 mg/kg, 50 mg/kg, 55 mg/kg, 60 mg/kg, 65 mg/kg, 70 mg/kg, 75 mg/kg, 80 mg/kg, 85 mg/kg, 90 mg/kg, 95 mg/kg, 100 mg/kg, 105 mg/kg, 110 mg/kg, 115 mg/kg, 120 mg/kg, 125 mg/kg, 130 mg/kg, 135 mg/kg, 140 mg/kg, 145 mg/kg, 150 mg/kg, 155 mg/kg, 160 mg/kg, 165 mg/kg, 170 mg/kg, 175 mg/kg, 180 mg/kg, 185 mg/kg, 190 mg/kg, 195 mg/kg, 200 mg/kg, and any fraction or integer in between any two of the preceding amounts.

In some embodiments, a NOS inhibitor composition comprising GW-274150 and/or a salt thereof, such as, for example, GW-274150 phosphate, may be administered as a suitable dosage form, as described herein, to treat, ameliorate, and/or reduce the severity of neuroblastoma or one or more symptoms thereof in a neuroblastoma subject. In certain embodiments, an nNOS inhibitor composition comprising GW-274150 and/or a salt thereof, such as, for example, GW-274150 phosphate, may be administered to a neuroblastoma subject to treat, ameliorate, and/or reduce the severity of neuroblastoma or one or more symptoms thereof in the neuroblastoma subject: (i) via a route selected from the group consisting of buccally, inhalation, intraarterially, intraarticularly, intracardially, intracerebroventricularly, intradermally, intramedullary, intramuscularly, intraperitoneally, intrathecally, intravenously, intraventricularly, nasally, ophthalmically, orally, rectally, sublabially, sublingually, subcutaneously, topically, and transdermally; and/or (ii) with a frequency selected from the group consisting of four times daily, three times daily, twice daily, once daily, once every two days, once every three days, once every four days, once every five days, once every six days, once a week, once every eight days, once every nine days, once every ten days, once every two weeks, once every three weeks, and once a month; and/or (iii) at a dosage (based on subject weight) selected from the group consisting of 0.1 μg/kg, 0.5 μg/kg, 1 μg/kg, 1.5 μg/kg, 2.0 μg/kg, 2.5 μg/kg, 3.0 μg/kg, 3.5 μg/kg, 4.0 μg/kg, 4.5 μg/kg, 5.0 μg/kg, 10 μg/kg, 15 μg/kg, 20 μg/kg, 25 μg/kg, 30 μg/kg, 35 μg/kg, 40 μg/kg, 45 μg/kg, 50 μg/kg, 55 μg/kg, 60 μg/kg, 65 μg/kg, 70 μg/kg, 75 μg/kg, 80 μg/kg, 85 μg/kg, 90 μg/kg, 95 μg/kg, 100 μg/kg, 150 μg/kg, 200 μg/kg, 250 μg/kg, 300 μg/kg, 350 μg/kg, 400 μg/kg, 450 μg/kg, 500 μg/kg, 550 μg/kg, 600 μg/kg, 650 μg/kg, 700 μg/kg, 750 μg/kg, 800 μg/kg, 850 μg/kg, 900 μg/kg, 1 mg/kg, 1.5 mg/kg, 2.0 mg/kg, 2.5 mg/kg, 3 mg/kg, 3.5 mg/kg, 4.0 mg/kg, 4.5 mg/kg, 5 mg/kg, 5.5 mg/kg, 6 mg/kg, 6.5 mg/kg, 7 mg/kg, 7.5 mg/kg, 8 mg/kg, 8.5 mg/kg, 9 mg/kg, 9.5 mg/kg, 10 mg/kg 10.5 mg/kg, 11 mg/kg, 11.5 mg/kg, 12 mg/kg, 12.5 mg/kg, 13 mg/kg, 13.5 mg/kg, 14 mg/kg, 14.5 mg/kg, 15 mg/kg, 16 mg/kg, 17 mg/kg, 18 mg/kg, 19 mg/kg, 20 mg/kg, 21 mg/kg, 22 mg/kg, 23 mg/kg, 24 mg/kg, 25 mg/kg, 30 mg/kg, 35 mg/kg, 40 mg/kg, 45 mg/kg, 50 mg/kg, 55 mg/kg, 60 mg/kg, 65 mg/kg, 70 mg/kg, 75 mg/kg, 80 mg/kg, 85 mg/kg, 90 mg/kg, 95 mg/kg, 100 mg/kg, 105 mg/kg, 110 mg/kg, 115 mg/kg, 120 mg/kg, 125 mg/kg, 130 mg/kg, 135 mg/kg, 140 mg/kg, 145 mg/kg, 150 mg/kg, 155 mg/kg, 160 mg/kg, 165 mg/kg, 170 mg/kg, 175 mg/kg, 180 mg/kg, 185 mg/kg, 190 mg/kg, 195 mg/kg, 200 mg/kg, and any fraction or integer in between any two of the preceding amounts.

In certain embodiments, a NOS inhibitor composition comprising L-NIL and/or a salt thereof, such as, for example, L-NIL hydrochloride, may be administered as a suitable dosage form, as described herein, to treat, ameliorate, and/or reduce the severity of neuroblastoma or one or more symptoms thereof in a neuroblastoma subject. In some embodiments, an nNOS inhibitor composition comprising L-NIL and/or a salt thereof, such as, for example, L-NIL hydrochloride, may be administered to a neuroblastoma subject to treat, ameliorate, and/or reduce the severity of neuroblastoma or one or more symptoms thereof in the neuroblastoma subject: (i) via a route selected from the group consisting of buccally, inhalation, intraarterially, intraarticularly, intracardially, intracerebroventricularly, intradermally, intramedullary, intramuscularly, intraperitoneally, intrathecally, intravenously, intraventricularly, nasally, ophthalmically, orally, rectally, sublabially, sublingually, subcutaneously, topically, and transdermally; and/or (ii) with a frequency selected from the group consisting of four times daily, three times daily, twice daily, once daily, once every two days, once every three days, once every four days, once every five days, once every six days, once a week, once every eight days, once every nine days, once every ten days, once every two weeks, once every three weeks, and once a month; and/or (iii) at a dosage (based on subject weight) selected from the group consisting of 0.1 μg/kg, 0.5 μg/kg, 1 μg/kg, 1.5 μg/kg, 2.0 μg/kg, 2.5 μg/kg, 3.0 μg/kg, 3.5 μg/kg, 4.0 μg/kg, 4.5 μg/kg, 5.0 μg/kg, 10 μg/kg, 15 μg/kg, 20 μg/kg, 25 μg/kg, 30 μg/kg, 35 μg/kg, 40 μg/kg, 45 μg/kg, 50 μg/kg, 55 μg/kg, 60 μg/kg, 65 μg/kg, 70 μg/kg, 75 μg/kg, 80 μg/kg, 85 μg/kg, 90 μg/kg, 95 μg/kg, 100 μg/kg, 150 μg/kg, 200 μg/kg, 250 μg/kg, 300 μg/kg, 350 μg/kg, 400 μg/kg, 450 μg/kg, 500 μg/kg, 550 μg/kg, 600 μg/kg, 650 μg/kg, 700 μg/kg, 750 μg/kg, 800 μg/kg, 850 μg/kg, 900 μg/kg, 1 mg/kg, 1.5 mg/kg, 2.0 mg/kg, 2.5 mg/kg, 3 mg/kg, 3.5 mg/kg, 4.0 mg/kg, 4.5 mg/kg, 5 mg/kg, 5.5 mg/kg, 6 mg/kg, 6.5 mg/kg, 7 mg/kg, 7.5 mg/kg, 8 mg/kg, 8.5 mg/kg, 9 mg/kg, 9.5 mg/kg, 10 mg/kg 10.5 mg/kg, 11 mg/kg, 11.5 mg/kg, 12 mg/kg, 12.5 mg/kg, 13 mg/kg, 13.5 mg/kg, 14 mg/kg, 14.5 mg/kg, 15 mg/kg, 16 mg/kg, 17 mg/kg, 18 mg/kg, 19 mg/kg, 20 mg/kg, 21 mg/kg, 22 mg/kg, 23 mg/kg, 24 mg/kg, 25 mg/kg, 30 mg/kg, 35 mg/kg, 40 mg/kg, 45 mg/kg, 50 mg/kg, 55 mg/kg, 60 mg/kg, 65 mg/kg, 70 mg/kg, 75 mg/kg, 80 mg/kg, 85 mg/kg, 90 mg/kg, 95 mg/kg, 100 mg/kg, 105 mg/kg, 110 mg/kg, 115 mg/kg, 120 mg/kg, 125 mg/kg, 130 mg/kg, 135 mg/kg, 140 mg/kg, 145 mg/kg, 150 mg/kg, 155 mg/kg, 160 mg/kg, 165 mg/kg, 170 mg/kg, 175 mg/kg, 180 mg/kg, 185 mg/kg, 190 mg/kg, 195 mg/kg, 200 mg/kg, and any fraction or integer in between any two of the preceding amounts.

In some embodiments, a NOS inhibitor composition comprising 1400W and/or a salt thereof, such as, for example, 1400W dihydrochloride, may be administered as a suitable dosage form, as described herein, to treat, ameliorate, and/or reduce the severity of neuroblastoma or one or more symptoms thereof in a neuroblastoma subject. In certain embodiments, an nNOS inhibitor composition comprising 1400W and/or a salt thereof, such as, for example, 1400W dihydrochloride, may be administered to a neuroblastoma subject to treat, ameliorate, and/or reduce the severity of neuroblastoma or one or more symptoms thereof in the neuroblastoma subject: (i) via a route selected from the group consisting of buccally, inhalation, intraarterially, intraarticularly, intracardially, intracerebroventricularly, intradermally, intramedullary, intramuscularly, intraperitoneally, intrathecally, intravenously, intraventricularly, nasally, ophthalmically, orally, rectally, sublabially, sublingually, subcutaneously, topically, and transdermally; and/or (ii) with a frequency selected from the group consisting of four times daily, three times daily, twice daily, once daily, once every two days, once every three days, once every four days, once every five days, once every six days, once a week, once every eight days, once every nine days, once every ten days, once every two weeks, once every three weeks, and once a month; and/or (iii) at a dosage (based on subject weight) selected from the group consisting of 0.1 μg/kg, 0.5 μg/kg, 1 μg/kg, 1.5 μg/kg, 2.0 μg/kg, 2.5 μg/kg, 3.0 μg/kg, 3.5 μg/kg, 4.0 μg/kg, 4.5 μg/kg, 5.0 μg/kg, 10 μg/kg, 15 μg/kg, 20 μg/kg, 25 μg/kg, 30 μg/kg, 35 μg/kg, 40 μg/kg, 45 μg/kg, 50 μg/kg, 55 μg/kg, 60 μg/kg, 65 μg/kg, 70 μg/kg, 75 μg/kg, 80 μg/kg, 85 μg/kg, 90 μg/kg, 95 μg/kg, 100 μg/kg, 150 μg/kg, 200 μg/kg, 250 μg/kg, 300 μg/kg, 350 μg/kg, 400 μg/kg, 450 μg/kg, 500 μg/kg, 550 μg/kg, 600 μg/kg, 650 μg/kg, 700 μg/kg, 750 μg/kg, 800 μg/kg, 850 μg/kg, 900 μg/kg, 1 mg/kg, 1.5 mg/kg, 2.0 mg/kg, 2.5 mg/kg, 3 mg/kg, 3.5 mg/kg, 4.0 mg/kg, 4.5 mg/kg, 5 mg/kg, 5.5 mg/kg, 6 mg/kg, 6.5 mg/kg, 7 mg/kg, 7.5 mg/kg, 8 mg/kg, 8.5 mg/kg, 9 mg/kg, 9.5 mg/kg, 10 mg/kg 10.5 mg/kg, 11 mg/kg, 11.5 mg/kg, 12 mg/kg, 12.5 mg/kg, 13 mg/kg, 13.5 mg/kg, 14 mg/kg, 14.5 mg/kg, 15 mg/kg, 16 mg/kg, 17 mg/kg, 18 mg/kg, 19 mg/kg, 20 mg/kg, 21 mg/kg, 22 mg/kg, 23 mg/kg, 24 mg/kg, 25 mg/kg, 30 mg/kg, 35 mg/kg, 40 mg/kg, 45 mg/kg, 50 mg/kg, 55 mg/kg, 60 mg/kg, 65 mg/kg, 70 mg/kg, 75 mg/kg, 80 mg/kg, 85 mg/kg, 90 mg/kg, 95 mg/kg, 100 mg/kg, 105 mg/kg, 110 mg/kg, 115 mg/kg, 120 mg/kg, 125 mg/kg, 130 mg/kg, 135 mg/kg, 140 mg/kg, 145 mg/kg, 150 mg/kg, 155 mg/kg, 160 mg/kg, 165 mg/kg, 170 mg/kg, 175 mg/kg, 180 mg/kg, 185 mg/kg, 190 mg/kg, 195 mg/kg, 200 mg/kg, and any fraction or integer in between any two of the preceding amounts.

In certain embodiments, a NOS inhibitor composition comprising OKN-007 and/or a salt thereof, such as, for example, OKN-007 sodium, may be administered as a suitable dosage form, as described herein, to treat, ameliorate, and/or reduce the severity of neuroblastoma or one or more symptoms thereof in a neuroblastoma subject. In some embodiments, an nNOS inhibitor composition comprising OKN-007 and/or a salt thereof, such as, for example, OKN-007 sodium, may be administered to a neuroblastoma subject to treat, ameliorate, and/or reduce the severity of neuroblastoma or one or more symptoms thereof in the neuroblastoma subject: (i) via a route selected from the group consisting of buccally, inhalation, intraarterially, intraarticularly, intracardially, intracerebroventricularly, intradermally, intramedullary, intramuscularly, intraperitoneally, intrathecally, intravenously, intraventricularly, nasally, ophthalmically, orally, rectally, sublabially, sublingually, subcutaneously, topically, and transdermally; and/or (ii) with a frequency selected from the group consisting of four times daily, three times daily, twice daily, once daily, once every two days, once every three days, once every four days, once every five days, once every six days, once a week, once every eight days, once every nine days, once every ten days, once every two weeks, once every three weeks, and once a month; and/or (iii) at a dosage (based on subject weight) selected from the group consisting of 0.1 μg/kg, 0.5 μg/kg, 1 μg/kg, 1.5 μg/kg, 2.0 μg/kg, 2.5 μg/kg, 3.0 μg/kg, 3.5 μg/kg, 4.0 μg/kg, 4.5 μg/kg, 5.0 μg/kg, 10 μg/kg, 15 μg/kg, 20 μg/kg, 25 μg/kg, 30 μg/kg, 35 μg/kg, 40 μg/kg, 45 μg/kg, 50 μg/kg, 55 μg/kg, 60 μg/kg, 65 μg/kg, 70 μg/kg, 75 μg/kg, 80 μg/kg, 85 μg/kg, 90 μg/kg, 95 μg/kg, 100 μg/kg, 150 μg/kg, 200 μg/kg, 250 μg/kg, 300 μg/kg, 350 μg/kg, 400 μg/kg, 450 μg/kg, 500 μg/kg, 550 μg/kg, 600 μg/kg, 650 μg/kg, 700 μg/kg, 750 μg/kg, 800 μg/kg, 850 μg/kg, 900 μg/kg, 1 mg/kg, 1.5 mg/kg, 2.0 mg/kg, 2.5 mg/kg, 3 mg/kg, 3.5 mg/kg, 4.0 mg/kg, 4.5 mg/kg, 5 mg/kg, 5.5 mg/kg, 6 mg/kg, 6.5 mg/kg, 7 mg/kg, 7.5 mg/kg, 8 mg/kg, 8.5 mg/kg, 9 mg/kg, 9.5 mg/kg, 10 mg/kg 10.5 mg/kg, 11 mg/kg, 11.5 mg/kg, 12 mg/kg, 12.5 mg/kg, 13 mg/kg, 13.5 mg/kg, 14 mg/kg, 14.5 mg/kg, 15 mg/kg, 16 mg/kg, 17 mg/kg, 18 mg/kg, 19 mg/kg, 20 mg/kg, 21 mg/kg, 22 mg/kg, 23 mg/kg, 24 mg/kg, 25 mg/kg, 30 mg/kg, 35 mg/kg, 40 mg/kg, 45 mg/kg, 50 mg/kg, 55 mg/kg, 60 mg/kg, 65 mg/kg, 70 mg/kg, 75 mg/kg, 80 mg/kg, 85 mg/kg, 90 mg/kg, 95 mg/kg, 100 mg/kg, 105 mg/kg, 110 mg/kg, 115 mg/kg, 120 mg/kg, 125 mg/kg, 130 mg/kg, 135 mg/kg, 140 mg/kg, 145 mg/kg, 150 mg/kg, 155 mg/kg, 160 mg/kg, 165 mg/kg, 170 mg/kg, 175 mg/kg, 180 mg/kg, 185 mg/kg, 190 mg/kg, 195 mg/kg, 200 mg/kg, and any fraction or integer in between any two of the preceding amounts.

In some embodiments, a NOS inhibitor composition comprising BYK-191023 and/or a salt thereof, such as, for example, BYK-191023 dihydrochloride, may be administered as a suitable dosage form, as described herein, to treat, ameliorate, and/or reduce the severity of neuroblastoma or one or more symptoms thereof in a neuroblastoma subject. In certain embodiments, an nNOS inhibitor composition comprising BYK-191023 and/or a salt thereof, such as, for example, BYK-191023 dihydrochloride, may be administered to a neuroblastoma subject to treat, ameliorate, and/or reduce the severity of neuroblastoma or one or more symptoms thereof in the neuroblastoma subject: (i) via a route selected from the group consisting of buccally, inhalation, intraarterially, intraarticularly, intracardially, intracerebroventricularly, intradermally, intramedullary, intramuscularly, intraperitoneally, intrathecally, intravenously, intraventricularly, nasally, ophthalmically, orally, rectally, sublabially, sublingually, subcutaneously, topically, and transdermally; and/or (ii) with a frequency selected from the group consisting of four times daily, three times daily, twice daily, once daily, once every two days, once every three days, once every four days, once every five days, once every six days, once a week, once every eight days, once every nine days, once every ten days, once every two weeks, once every three weeks, and once a month; and/or (iii) at a dosage (based on subject weight) selected from the group consisting of 0.1 μg/kg, 0.5 μg/kg, 1 μg/kg, 1.5 μg/kg, 2.0 μg/kg, 2.5 μg/kg, 3.0 μg/kg, 3.5 μg/kg, 4.0 μg/kg, 4.5 μg/kg, 5.0 μg/kg, 10 μg/kg, 15 μg/kg, 20 μg/kg, 25 μg/kg, 30 μg/kg, 35 μg/kg, 40 μg/kg, 45 μg/kg, 50 μg/kg, 55 μg/kg, 60 μg/kg, 65 μg/kg, 70 μg/kg, 75 μg/kg, 80 μg/kg, 85 μg/kg, 90 μg/kg, 95 μg/kg, 100 μg/kg, 150 μg/kg, 200 μg/kg, 250 μg/kg, 300 μg/kg, 350 μg/kg, 400 μg/kg, 450 μg/kg, 500 μg/kg, 550 μg/kg, 600 μg/kg, 650 μg/kg, 700 μg/kg, 750 μg/kg, 800 μg/kg, 850 μg/kg, 900 μg/kg, 1 mg/kg, 1.5 mg/kg, 2.0 mg/kg, 2.5 mg/kg, 3 mg/kg, 3.5 mg/kg, 4.0 mg/kg, 4.5 mg/kg, 5 mg/kg, 5.5 mg/kg, 6 mg/kg, 6.5 mg/kg, 7 mg/kg, 7.5 mg/kg, 8 mg/kg, 8.5 mg/kg, 9 mg/kg, 9.5 mg/kg, 10 mg/kg 10.5 mg/kg, 11 mg/kg, 11.5 mg/kg, 12 mg/kg, 12.5 mg/kg, 13 mg/kg, 13.5 mg/kg, 14 mg/kg, 14.5 mg/kg, 15 mg/kg, 16 mg/kg, 17 mg/kg, 18 mg/kg, 19 mg/kg, 20 mg/kg, 21 mg/kg, 22 mg/kg, 23 mg/kg, 24 mg/kg, 25 mg/kg, 30 mg/kg, 35 mg/kg, 40 mg/kg, 45 mg/kg, 50 mg/kg, 55 mg/kg, 60 mg/kg, 65 mg/kg, 70 mg/kg, 75 mg/kg, 80 mg/kg, 85 mg/kg, 90 mg/kg, 95 mg/kg, 100 mg/kg, 105 mg/kg, 110 mg/kg, 115 mg/kg, 120 mg/kg, 125 mg/kg, 130 mg/kg, 135 mg/kg, 140 mg/kg, 145 mg/kg, 150 mg/kg, 155 mg/kg, 160 mg/kg, 165 mg/kg, 170 mg/kg, 175 mg/kg, 180 mg/kg, 185 mg/kg, 190 mg/kg, 195 mg/kg, 200 mg/kg, and any fraction or integer in between any two of the preceding amounts.

In certain embodiments, a NOS inhibitor composition comprising CM544 and/or a salt thereof, such as, for example, CM544 dihydrochloride, may be administered as a suitable dosage form, as described herein, to treat, ameliorate, and/or reduce the severity of neuroblastoma or one or more symptoms thereof in a neuroblastoma subject. In some embodiments, an nNOS inhibitor composition comprising CM544 and/or a salt thereof, such as, for example, CM544 dihydrochloride, may be administered to a neuroblastoma subject to treat, ameliorate, and/or reduce the severity of neuroblastoma or one or more symptoms thereof in the neuroblastoma subject: (i) via a route selected from the group consisting of buccally, inhalation, intraarterially, intraarticularly, intracardially, intracerebroventricularly, intradermally, intramedullary, intramuscularly, intraperitoneally, intrathecally, intravenously, intraventricularly, nasally, ophthalmically, orally, rectally, sublabially, sublingually, subcutaneously, topically, and transdermally; and/or (ii) with a frequency selected from the group consisting of four times daily, three times daily, twice daily, once daily, once every two days, once every three days, once every four days, once every five days, once every six days, once a week, once every eight days, once every nine days, once every ten days, once every two weeks, once every three weeks, and once a month; and/or (iii) at a dosage (based on subject weight) selected from the group consisting of 0.1 μg/kg, 0.5 μg/kg, 1 μg/kg, 1.5 μg/kg, 2.0 μg/kg, 2.5 μg/kg, 3.0 μg/kg, 3.5 μg/kg, 4.0 μg/kg, 4.5 μg/kg, 5.0 μg/kg, 10 μg/kg, 15 μg/kg, 20 μg/kg, 25 μg/kg, 30 μg/kg, 35 μg/kg, 40 μg/kg, 45 μg/kg, 50 μg/kg, 55 μg/kg, 60 μg/kg, 65 μg/kg, 70 μg/kg, 75 μg/kg, 80 μg/kg, 85 μg/kg, 90 μg/kg, 95 μg/kg, 100 μg/kg, 150 μg/kg, 200 μg/kg, 250 μg/kg, 300 μg/kg, 350 μg/kg, 400 μg/kg, 450 μg/kg, 500 μg/kg, 550 μg/kg, 600 μg/kg, 650 μg/kg, 700 μg/kg, 750 μg/kg, 800 μg/kg, 850 μg/kg, 900 μg/kg, 1 mg/kg, 1.5 mg/kg, 2.0 mg/kg, 2.5 mg/kg, 3 mg/kg, 3.5 mg/kg, 4.0 mg/kg, 4.5 mg/kg, 5 mg/kg, 5.5 mg/kg, 6 mg/kg, 6.5 mg/kg, 7 mg/kg, 7.5 mg/kg, 8 mg/kg, 8.5 mg/kg, 9 mg/kg, 9.5 mg/kg, 10 mg/kg 10.5 mg/kg, 11 mg/kg, 11.5 mg/kg, 12 mg/kg, 12.5 mg/kg, 13 mg/kg, 13.5 mg/kg, 14 mg/kg, 14.5 mg/kg, 15 mg/kg, 16 mg/kg, 17 mg/kg, 18 mg/kg, 19 mg/kg, 20 mg/kg, 21 mg/kg, 22 mg/kg, 23 mg/kg, 24 mg/kg, 25 mg/kg, 30 mg/kg, 35 mg/kg, 40 mg/kg, 45 mg/kg, 50 mg/kg, 55 mg/kg, 60 mg/kg, 65 mg/kg, 70 mg/kg, 75 mg/kg, 80 mg/kg, 85 mg/kg, 90 mg/kg, 95 mg/kg, 100 mg/kg, 105 mg/kg, 110 mg/kg, 115 mg/kg, 120 mg/kg, 125 mg/kg, 130 mg/kg, 135 mg/kg, 140 mg/kg, 145 mg/kg, 150 mg/kg, 155 mg/kg, 160 mg/kg, 165 mg/kg, 170 mg/kg, 175 mg/kg, 180 mg/kg, 185 mg/kg, 190 mg/kg, 195 mg/kg, 200 mg/kg, and any fraction or integer in between any two of the preceding amounts.

In certain embodiments, an nNOS inhibitor composition comprising one or more dsRNAs each comprising at least 30 base pairs, may be administered as a suitable dosage form, as described herein, to treat, ameliorate, and/or reduce the severity of neuroblastoma or one or more symptoms thereof in a neuroblastoma subject. In some embodiments, an nNOS inhibitor composition comprising one or more dsRNAs, each comprising at least 30 base pairs, may be administered to a neuroblastoma subject to treat, ameliorate, and/or reduce the severity of neuroblastoma or one or more symptoms thereof in the neuroblastoma subject: (i) via a route selected from the group consisting of buccally, inhalation, intraarterially, intraarticularly, intracardially, intracerebroventricularly, intradermally, intramedullary, intramuscularly, intraperitoneally, intrathecally, intravenously, intraventricularly, nasally, ophthalmically, orally, rectally, sublabially, sublingually, subcutaneously, topically, and transdermally; and/or (ii) with a frequency selected from the group consisting of four times daily, three times daily, twice daily, once daily, once every two days, once every three days, once every four days, once every five days, once every six days, once a week, once every eight days, once every nine days, once every ten days, once every two weeks, once every three weeks, and once a month; and/or (iii) at a dosage (based on subject weight) selected from the group consisting of 0.1 μg/kg, 0.5 μg/kg, 1 μg/kg, 1.5 μg/kg, 2.0 μg/kg, 2.5 μg/kg, 3.0 μg/kg, 3.5 μg/kg, 4.0 μg/kg, 4.5 μg/kg, 5.0 μg/kg, 10 μg/kg, 15 μg/kg, 20 μg/kg, 25 μg/kg, 30 μg/kg, 35 μg/kg, 40 μg/kg, 45 μg/kg, 50 μg/kg, 55 μg/kg, 60 μg/kg, 65 μg/kg, 70 μg/kg, 75 μg/kg, 80 μg/kg, 85 μg/kg, 90 μg/kg, 95 μg/kg, 100 μg/kg, 150 μg/kg, 200 μg/kg, 250 μg/kg, 300 μg/kg, 350 μg/kg, 400 μg/kg, 450 μg/kg, 500 μg/kg, 550 μg/kg, 600 μg/kg, 650 μg/kg, 700 μg/kg, 750 μg/kg, 800 μg/kg, 850 μg/kg, 900 μg/kg, 1 mg/kg, 1.5 mg/kg, 2.0 mg/kg, 2.5 mg/kg, 3 mg/kg, 3.5 mg/kg, 4.0 mg/kg, 4.5 mg/kg, 5 mg/kg, 5.5 mg/kg, 6 mg/kg, 6.5 mg/kg, 7 mg/kg, 7.5 mg/kg, 8 mg/kg, 8.5 mg/kg, 9 mg/kg, 9.5 mg/kg, 10 mg/kg 10.5 mg/kg, 11 mg/kg, 11.5 mg/kg, 12 mg/kg, 12.5 mg/kg, 13 mg/kg, 13.5 mg/kg, 14 mg/kg, 14.5 mg/kg, 15 mg/kg, 16 mg/kg, 17 mg/kg, 18 mg/kg, 19 mg/kg, 20 mg/kg, 21 mg/kg, 22 mg/kg, 23 mg/kg, 24 mg/kg, 25 mg/kg, 30 mg/kg, 35 mg/kg, 40 mg/kg, 45 mg/kg, 50 mg/kg, 55 mg/kg, 60 mg/kg, 65 mg/kg, 70 mg/kg, 75 mg/kg, 80 mg/kg, 85 mg/kg, 90 mg/kg, 95 mg/kg, 100 mg/kg, 105 mg/kg, 110 mg/kg, 115 mg/kg, 120 mg/kg, 125 mg/kg, 130 mg/kg, 135 mg/kg, 140 mg/kg, 145 mg/kg, 150 mg/kg, 155 mg/kg, 160 mg/kg, 165 mg/kg, 170 mg/kg, 175 mg/kg, 180 mg/kg, 185 mg/kg, 190 mg/kg, 195 mg/kg, 200 mg/kg, and any fraction or integer in between any two of the preceding amounts.

In some embodiments, an nNOS inhibitor composition comprising one or more siRNAs, may be administered as a suitable dosage form, as described herein, to treat, ameliorate, and/or reduce the severity of neuroblastoma or one or more symptoms thereof in a neuroblastoma subject. In certain embodiments, an nNOS inhibitor composition comprising one or more siRNAs selected from the group consisting of SEQ ID NO. 4, SEQ ID NO. 5, SEQ ID NO. 6, SEQ ID NO. 7, SEQ ID NO. 8, and any combination thereof, may be administered as a suitable dosage form, as described herein, to treat, ameliorate, and/or reduce the severity of neuroblastoma or one or more symptoms thereof in a neuroblastoma subject. In some embodiments, an nNOS inhibitor composition comprising one or more siRNAs selected from the group consisting of SEQ ID NO. 4, SEQ ID NO. 5, SEQ ID NO. 6, SEQ ID NO. 7, SEQ ID NO. 8, and any combination thereof, may be administered to a neuroblastoma subject to treat, ameliorate, and/or reduce the severity of neuroblastoma or one or more symptoms thereof in the neuroblastoma subject: (i) via a route selected from the group consisting of buccally, inhalation, intraarterially, intraarticularly, intracardially, intracerebroventricularly, intradermally, intramedullary, intramuscularly, intraperitoneally, intrathecally, intravenously, intraventricularly, nasally, ophthalmically, orally, rectally, sublabially, sublingually, subcutaneously, topically, and transdermally; and/or (ii) with a frequency selected from the group consisting of four times daily, three times daily, twice daily, once daily, once every two days, once every three days, once every four days, once every five days, once every six days, once a week, once every eight days, once every nine days, once every ten days, once every two weeks, once every three weeks, and once a month; and/or (iii) at a dosage (based on subject weight) selected from the group consisting of 0.1 μg/kg, 0.5 μg/kg, 1 μg/kg, 1.5 μg/kg, 2.0 μg/kg, 2.5 μg/kg, 3.0 μg/kg, 3.5 μg/kg, 4.0 μg/kg, 4.5 μg/kg, 5.0 μg/kg, 10 μg/kg, 15 μg/kg, 20 μg/kg, 25 μg/kg, 30 μg/kg, 35 μg/kg, 40 μg/kg, 45 μg/kg, 50 μg/kg, 55 μg/kg, 60 μg/kg, 65 μg/kg, 70 μg/kg, 75 μg/kg, 80 μg/kg, 85 μg/kg, 90 μg/kg, 95 μg/kg, 100 μg/kg, 150 μg/kg, 200 μg/kg, 250 μg/kg, 300 μg/kg, 350 μg/kg, 400 μg/kg, 450 μg/kg, 500 μg/kg, 550 μg/kg, 600 μg/kg, 650 μg/kg, 700 μg/kg, 750 μg/kg, 800 μg/kg, 850 μg/kg, 900 μg/kg, 1 mg/kg, 1.5 mg/kg, 2.0 mg/kg, 2.5 mg/kg, 3 mg/kg, 3.5 mg/kg, 4.0 mg/kg, 4.5 mg/kg, 5 mg/kg, 5.5 mg/kg, 6 mg/kg, 6.5 mg/kg, 7 mg/kg, 7.5 mg/kg, 8 mg/kg, 8.5 mg/kg, 9 mg/kg, 9.5 mg/kg, 10 mg/kg 10.5 mg/kg, 11 mg/kg, 11.5 mg/kg, 12 mg/kg, 12.5 mg/kg, 13 mg/kg, 13.5 mg/kg, 14 mg/kg, 14.5 mg/kg, 15 mg/kg, 16 mg/kg, 17 mg/kg, 18 mg/kg, 19 mg/kg, 20 mg/kg, 21 mg/kg, 22 mg/kg, 23 mg/kg, 24 mg/kg, 25 mg/kg, 30 mg/kg, 35 mg/kg, 40 mg/kg, 45 mg/kg, 50 mg/kg, 55 mg/kg, 60 mg/kg, 65 mg/kg, 70 mg/kg, 75 mg/kg, 80 mg/kg, 85 mg/kg, 90 mg/kg, 95 mg/kg, 100 mg/kg, 105 mg/kg, 110 mg/kg, 115 mg/kg, 120 mg/kg, 125 mg/kg, 130 mg/kg, 135 mg/kg, 140 mg/kg, 145 mg/kg, 150 mg/kg, 155 mg/kg, 160 mg/kg, 165 mg/kg, 170 mg/kg, 175 mg/kg, 180 mg/kg, 185 mg/kg, 190 mg/kg, 195 mg/kg, 200 mg/kg, and any fraction or integer in between any two of the preceding amounts.

In some embodiments, an nNOS inhibitor composition comprising one or more shRNAs, may be administered as a suitable dosage form, as described herein, to treat, ameliorate, and/or reduce the severity of neuroblastoma or one or more symptoms thereof in a neuroblastoma subject. In certain embodiments, an nNOS inhibitor composition comprising one or more shRNAs selected from the group consisting of SEQ ID NO. 9, SEQ ID NO. 10, SEQ ID NO. 11, SEQ ID NO. 12, SEQ ID NO. 13, SEQ ID NO. 14, and any combination thereof, may be administered as a suitable dosage form, as described herein, to treat, ameliorate, and/or reduce the severity of neuroblastoma or one or more symptoms thereof in a neuroblastoma subject. In some embodiments, an nNOS inhibitor composition comprising one or more shRNAs selected from the group consisting of SEQ ID NO. 9, SEQ ID NO. 10, SEQ ID NO. 11, SEQ ID NO. 12, SEQ ID NO. 13, SEQ ID NO. 14, and any combination thereof, may be administered to a neuroblastoma subject to treat, ameliorate, and/or reduce the severity of neuroblastoma or one or more symptoms thereof in the neuroblastoma subject: (i) via a route selected from the group consisting of buccally, inhalation, intraarterially, intraarticularly, intracardially, intracerebroventricularly, intradermally, intramedullary, intramuscularly, intraperitoneally, intrathecally, intravenously, intraventricularly, nasally, ophthalmically, orally, rectally, sublabially, sublingually, subcutaneously, topically, and transdermally; and/or (ii) with a frequency selected from the group consisting of four times daily, three times daily, twice daily, once daily, once every two days, once every three days, once every four days, once every five days, once every six days, once a week, once every eight days, once every nine days, once every ten days, once every two weeks, once every three weeks, and once a month; and/or (iii) at a dosage (based on subject weight) selected from the group consisting of 0.1 μg/kg, 0.5 μg/kg, 1 μg/kg, 1.5 μg/kg, 2.0 μg/kg, 2.5 μg/kg, 3.0 μg/kg, 3.5 μg/kg, 4.0 μg/kg, 4.5 μg/kg, 5.0 μg/kg, 10 μg/kg, 15 μg/kg, 20 μg/kg, 25 μg/kg, 30 μg/kg, 35 μg/kg, 40 μg/kg, 45 μg/kg, 50 μg/kg, 55 μg/kg, 60 μg/kg, 65 μg/kg, 70 μg/kg, 75 μg/kg, 80 μg/kg, 85 μg/kg, 90 μg/kg, 95 μg/kg, 100 μg/kg, 150 μg/kg, 200 μg/kg, 250 μg/kg, 300 μg/kg, 350 μg/kg, 400 μg/kg, 450 μg/kg, 500 μg/kg, 550 μg/kg, 600 μg/kg, 650 μg/kg, 700 μg/kg, 750 μg/kg, 800 μg/kg, 850 μg/kg, 900 μg/kg, 1 mg/kg, 1.5 mg/kg, 2.0 mg/kg, 2.5 mg/kg, 3 mg/kg, 3.5 mg/kg, 4.0 mg/kg, 4.5 mg/kg, 5 mg/kg, 5.5 mg/kg, 6 mg/kg, 6.5 mg/kg, 7 mg/kg, 7.5 mg/kg, 8 mg/kg, 8.5 mg/kg, 9 mg/kg, 9.5 mg/kg, 10 mg/kg 10.5 mg/kg, 11 mg/kg, 11.5 mg/kg, 12 mg/kg, 12.5 mg/kg, 13 mg/kg, 13.5 mg/kg, 14 mg/kg, 14.5 mg/kg, 15 mg/kg, 16 mg/kg, 17 mg/kg, 18 mg/kg, 19 mg/kg, 20 mg/kg, 21 mg/kg, 22 mg/kg, 23 mg/kg, 24 mg/kg, 25 mg/kg, 30 mg/kg, 35 mg/kg, 40 mg/kg, 45 mg/kg, 50 mg/kg, 55 mg/kg, 60 mg/kg, 65 mg/kg, 70 mg/kg, 75 mg/kg, 80 mg/kg, 85 mg/kg, 90 mg/kg, 95 mg/kg, 100 mg/kg, 105 mg/kg, 110 mg/kg, 115 mg/kg, 120 mg/kg, 125 mg/kg, 130 mg/kg, 135 mg/kg, 140 mg/kg, 145 mg/kg, 150 mg/kg, 155 mg/kg, 160 mg/kg, 165 mg/kg, 170 mg/kg, 175 mg/kg, 180 mg/kg, 185 mg/kg, 190 mg/kg, 195 mg/kg, 200 mg/kg, and any fraction or integer in between any two of the preceding amounts.

In some embodiments, an nNOS inhibitor composition comprising one or more miRNAs, may be administered as a suitable dosage form, as described herein, to treat, ameliorate, and/or reduce the severity of neuroblastoma or one or more symptoms thereof in a neuroblastoma subject. In certain embodiments, an nNOS inhibitor composition comprising one or more miRNAs selected from the group consisting of miR-146a, hsa-miR-146a-5p, hsa-miR-1273f, hsa-miR-143-3p, hsa-miR-147a, hsa-miR-3622a-5p, hsa-miR-3911, hsa-miR-4269, hsa-miR-4708-5p, hsa-miR-4710, hsa-miR-4770, hsa-miR-4792, hsa-miR-574-5p, hsa-miR-6088, hsa-miR-644a, hsa-miR-6715b-5p, hsa-miR-6768-5p, hsa-miR-6867-5p, hsa-miR-7855-5p, hsa-miR-8485, and any combination thereof, may be administered as a suitable dosage form, as described herein, to treat, ameliorate, and/or reduce the severity of neuroblastoma or one or more symptoms thereof in a neuroblastoma subject. In some embodiments, an nNOS inhibitor composition comprising one or more miRNAs selected from the group consisting of miR-146a, hsa-miR-146a-5p, hsa-miR-1273f, hsa-miR-143-3p, hsa-miR-147a, hsa-miR-3622a-5p, hsa-miR-3911, hsa-miR-4269, hsa-miR-4708-5p, hsa-miR-4710, hsa-miR-4770, hsa-miR-4792, hsa-miR-574-5p, hsa-miR-6088, hsa-miR-644a, hsa-miR-6715b-5p, hsa-miR-6768-5p, hsa-miR-6867-5p, hsa-miR-7855-5p, hsa-miR-8485, and any combination thereof, may be administered to a neuroblastoma subject to treat, ameliorate, and/or reduce the severity of neuroblastoma or one or more symptoms thereof in the neuroblastoma subject: (i) via a route selected from the group consisting of buccally, inhalation, intraarterially, intraarticularly, intracardially, intracerebroventricularly, intradermally, intramedullary, intramuscularly, intraperitoneally, intrathecally, intravenously, intraventricularly, nasally, ophthalmically, orally, rectally, sublabially, sublingually, subcutaneously, topically, and transdermally; and/or (ii) with a frequency selected from the group consisting of four times daily, three times daily, twice daily, once daily, once every two days, once every three days, once every four days, once every five days, once every six days, once a week, once every eight days, once every nine days, once every ten days, once every two weeks, once every three weeks, and once a month; and/or (iii) at a dosage (based on subject weight) selected from the group consisting of 0.1 μg/kg, 0.5 μg/kg, 1 μg/kg, 1.5 μg/kg, 2.0 μg/kg, 2.5 μg/kg, 3.0 μg/kg, 3.5 μg/kg, 4.0 μg/kg, 4.5 μg/kg, 5.0 μg/kg, 10 μg/kg, 15 μg/kg, 20 μg/kg, 25 μg/kg, 30 μg/kg, 35 μg/kg, 40 μg/kg, 45 μg/kg, 50 μg/kg, 55 μg/kg, 60 μg/kg, 65 μg/kg, 70 μg/kg, 75 μg/kg, 80 μg/kg, 85 μg/kg, 90 μg/kg, 95 μg/kg, 100 μg/kg, 150 μg/kg, 200 μg/kg, 250 μg/kg, 300 μg/kg, 350 μg/kg, 400 μg/kg, 450 μg/kg, 500 μg/kg, 550 μg/kg, 600 μg/kg, 650 μg/kg, 700 μg/kg, 750 μg/kg, 800 μg/kg, 850 μg/kg, 900 μg/kg, 1 mg/kg, 1.5 mg/kg, 2.0 mg/kg, 2.5 mg/kg, 3 mg/kg, 3.5 mg/kg, 4.0 mg/kg, 4.5 mg/kg, 5 mg/kg, 5.5 mg/kg, 6 mg/kg, 6.5 mg/kg, 7 mg/kg, 7.5 mg/kg, 8 mg/kg, 8.5 mg/kg, 9 mg/kg, 9.5 mg/kg, 10 mg/kg 10.5 mg/kg, 11 mg/kg, 11.5 mg/kg, 12 mg/kg, 12.5 mg/kg, 13 mg/kg, 13.5 mg/kg, 14 mg/kg, 14.5 mg/kg, 15 mg/kg, 16 mg/kg, 17 mg/kg, 18 mg/kg, 19 mg/kg, 20 mg/kg, 21 mg/kg, 22 mg/kg, 23 mg/kg, 24 mg/kg, 25 mg/kg, 30 mg/kg, 35 mg/kg, 40 mg/kg, 45 mg/kg, 50 mg/kg, 55 mg/kg, 60 mg/kg, 65 mg/kg, 70 mg/kg, 75 mg/kg, 80 mg/kg, 85 mg/kg, 90 mg/kg, 95 mg/kg, 100 mg/kg, 105 mg/kg, 110 mg/kg, 115 mg/kg, 120 mg/kg, 125 mg/kg, 130 mg/kg, 135 mg/kg, 140 mg/kg, 145 mg/kg, 150 mg/kg, 155 mg/kg, 160 mg/kg, 165 mg/kg, 170 mg/kg, 175 mg/kg, 180 mg/kg, 185 mg/kg, 190 mg/kg, 195 mg/kg, 200 mg/kg, and any fraction or integer in between any two of the preceding amounts.

In some embodiments, an nNOS inhibitor composition comprising one or more CRISPR/Cas systems, may be administered as a suitable dosage form, as described herein, to treat, ameliorate, and/or reduce the severity of neuroblastoma or one or more symptoms thereof in a neuroblastoma subject. In certain embodiments, an nNOS inhibitor composition comprising one or more CRISPR/Cas systems comprising an endonuclease selected from the group consisting of Cas1, Cas2, Cas3, Cas4, Cas5, Cas6, Cas7, Cas8a, Cas8b, Cas8c, Cas9, Cas10, Cas10d, Cas12, Cas12a, Cas12b, Cas12c, Cas12d, Cas12e, Cas12f, Cas12g, Cas12h, Cas12i, Cas12k, C2c4, C2c8, C2c9, Cas13, Cas13a, Cas13b, Cas13c, Cas13d, Cas13x.1, Cse1, Cse2, Csy1, Csy2, Csy3, GSU0054, Csm2, Cmr5, Csx10, Csx11, Csf1, and Csn2, may be administered as a suitable dosage form, as described herein, to treat, ameliorate, and/or reduce the severity of neuroblastoma or one or more symptoms thereof in a neuroblastoma subject. In some embodiments, an nNOS inhibitor composition comprising one or more CRISPR/Cas systems comprising an endonuclease selected from the group consisting of Cas1, Cas2, Cas3, Cas4, Cas5, Cas6, Cas7, Cas8a, Cas8b, Cas8c, Cas9, Cas10, Cas10d, Cas12, Cas12a, Cas12b, Cas12c, Cas12d, Cas12e, Cas12f, Cas12g, Cas12h, Cas12i, Cas12k, C2c4, C2c8, C2c9, Cas13, Cas13a, Cas13b, Cas13c, Cas13d, Cas13x.1, Cse1, Cse2, Csy1, Csy2, Csy3, GSU0054, Csm2, Cmr5, Csx10, Csx11, Csf1, and Csn2, may be administered to a neuroblastoma subject to treat, ameliorate, and/or reduce the severity of neuroblastoma or one or more symptoms thereof in the neuroblastoma subject: (i) via a route selected from the group consisting of buccally, inhalation, intraarterially, intraarticularly, intracardially, intracerebroventricularly, intradermally, intramedullary, intramuscularly, intraperitoneally, intrathecally, intravenously, intraventricularly, nasally, ophthalmically, orally, rectally, sublabially, sublingually, subcutaneously, topically, and transdermally; and/or (ii) with a frequency selected from the group consisting of four times daily, three times daily, twice daily, once daily, once every two days, once every three days, once every four days, once every five days, once every six days, once a week, once every eight days, once every nine days, once every ten days, once every two weeks, once every three weeks, and once a month; and/or (iii) at a dosage (based on subject weight) selected from the group consisting of 0.1 μg/kg, 0.5 μg/kg, 1 g/kg, 1.5 μg/kg, 2.0 μg/kg, 2.5 μg/kg, 3.0 μg/kg, 3.5 μg/kg, 4.0 μg/kg, 4.5 μg/kg, 5.0 μg/kg, 10 μg/kg, 15 μg/kg, 20 μg/kg, 25 μg/kg, 30 μg/kg, 35 μg/kg, 40 μg/kg, 45 μg/kg, 50 μg/kg, 55 μg/kg, 60 μg/kg, 65 μg/kg, 70 μg/kg, 75 μg/kg, 80 μg/kg, 85 μg/kg, 90 μg/kg, 95 μg/kg, 100 μg/kg, 150 μg/kg, 200 μg/kg, 250 μg/kg, 300 μg/kg, 350 μg/kg, 400 μg/kg, 450 μg/kg, 500 μg/kg, 550 μg/kg, 600 μg/kg, 650 μg/kg, 700 μg/kg, 750 μg/kg, 800 μg/kg, 850 μg/kg, 900 μg/kg, 1 mg/kg, 1.5 mg/kg, 2.0 mg/kg, 2.5 mg/kg, 3 mg/kg, 3.5 mg/kg, 4.0 mg/kg, 4.5 mg/kg, 5 mg/kg, 5.5 mg/kg, 6 mg/kg, 6.5 mg/kg, 7 mg/kg, 7.5 mg/kg, 8 mg/kg, 8.5 mg/kg, 9 mg/kg, 9.5 mg/kg, 10 mg/kg 10.5 mg/kg, 11 mg/kg, 11.5 mg/kg, 12 mg/kg, 12.5 mg/kg, 13 mg/kg, 13.5 mg/kg, 14 mg/kg, 14.5 mg/kg, 15 mg/kg, 16 mg/kg, 17 mg/kg, 18 mg/kg, 19 mg/kg, 20 mg/kg, 21 mg/kg, 22 mg/kg, 23 mg/kg, 24 mg/kg, 25 mg/kg, 30 mg/kg, 35 mg/kg, 40 mg/kg, 45 mg/kg, 50 mg/kg, 55 mg/kg, 60 mg/kg, 65 mg/kg, 70 mg/kg, 75 mg/kg, 80 mg/kg, 85 mg/kg, 90 mg/kg, 95 mg/kg, 100 mg/kg, 105 mg/kg, 110 mg/kg, 115 mg/kg, 120 mg/kg, 125 mg/kg, 130 mg/kg, 135 mg/kg, 140 mg/kg, 145 mg/kg, 150 mg/kg, 155 mg/kg, 160 mg/kg, 165 mg/kg, 170 mg/kg, 175 mg/kg, 180 mg/kg, 185 mg/kg, 190 mg/kg, 195 mg/kg, 200 mg/kg, and any fraction or integer in between any two of the preceding amounts.

In some embodiments, an nNOS inhibitor composition comprising one or more antibodies, may be administered as a suitable dosage form, as described herein, to treat, ameliorate, and/or reduce the severity of neuroblastoma or one or more symptoms thereof in a neuroblastoma subject. In certain embodiments, an nNOS inhibitor composition comprising one or more antibodies selected from the group consisting of 37-2800 (from ThermoFisher), EP1855Y (from abeam), EPR 24351-6 (from abeam), and any combination thereof, may be administered as a suitable dosage form, as described herein, to treat, ameliorate, and/or reduce the severity of neuroblastoma or one or more symptoms thereof in a neuroblastoma subject. In some embodiments, an nNOS inhibitor composition comprising one or more antibodies selected from the group consisting of 37-2800 (from ThermoFisher), EP1855Y (from abeam), EPR 24351-6 (from abeam), and any combination thereof, may be administered to a neuroblastoma subject to treat, ameliorate, and/or reduce the severity of neuroblastoma or one or more symptoms thereof in the neuroblastoma subject: (i) via a route selected from the group consisting of buccally, inhalation, intraarterially, intraarticularly, intracardially, intracerebroventricularly, intradermally, intramedullary, intramuscularly, intraperitoneally, intrathecally, intravenously, intraventricularly, nasally, ophthalmically, orally, rectally, sublabially, sublingually, subcutaneously, topically, and transdermally; and/or (ii) with a frequency selected from the group consisting of four times daily, three times daily, twice daily, once daily, once every two days, once every three days, once every four days, once every five days, once every six days, once a week, once every eight days, once every nine days, once every ten days, once every two weeks, once every three weeks, and once a month; and/or (iii) at a dosage (based on subject weight) selected from the group consisting of 0.1 μg/kg, 0.5 μg/kg, 1 μg/kg, 1.5 μg/kg, 2.0 μg/kg, 2.5 μg/kg, 3.0 μg/kg, 3.5 μg/kg, 4.0 μg/kg, 4.5 μg/kg, 5.0 μg/kg, 10 μg/kg, 15 μg/kg, 20 μg/kg, 25 μg/kg, 30 μg/kg, 35 μg/kg, 40 μg/kg, 45 μg/kg, 50 μg/kg, 55 μg/kg, 60 μg/kg, 65 μg/kg, 70 μg/kg, 75 μg/kg, 80 μg/kg, 85 μg/kg, 90 μg/kg, 95 μg/kg, 100 μg/kg, 150 μg/kg, 200 μg/kg, 250 μg/kg, 300 μg/kg, 350 μg/kg, 400 μg/kg, 450 μg/kg, 500 μg/kg, 550 μg/kg, 600 μg/kg, 650 μg/kg, 700 μg/kg, 750 μg/kg, 800 μg/kg, 850 μg/kg, 900 μg/kg, 1 mg/kg, 1.5 mg/kg, 2.0 mg/kg, 2.5 mg/kg, 3 mg/kg, 3.5 mg/kg, 4.0 mg/kg, 4.5 mg/kg, 5 mg/kg, 5.5 mg/kg, 6 mg/kg, 6.5 mg/kg, 7 mg/kg, 7.5 mg/kg, 8 mg/kg, 8.5 mg/kg, 9 mg/kg, 9.5 mg/kg, 10 mg/kg 10.5 mg/kg, 11 mg/kg, 11.5 mg/kg, 12 mg/kg, 12.5 mg/kg, 13 mg/kg, 13.5 mg/kg, 14 mg/kg, 14.5 mg/kg, 15 mg/kg, 16 mg/kg, 17 mg/kg, 18 mg/kg, 19 mg/kg, 20 mg/kg, 21 mg/kg, 22 mg/kg, 23 mg/kg, 24 mg/kg, 25 mg/kg, 30 mg/kg, 35 mg/kg, 40 mg/kg, 45 mg/kg, 50 mg/kg, 55 mg/kg, 60 mg/kg, 65 mg/kg, 70 mg/kg, 75 mg/kg, 80 mg/kg, 85 mg/kg, 90 mg/kg, 95 mg/kg, 100 mg/kg, 105 mg/kg, 110 mg/kg, 115 mg/kg, 120 mg/kg, 125 mg/kg, 130 mg/kg, 135 mg/kg, 140 mg/kg, 145 mg/kg, 150 mg/kg, 155 mg/kg, 160 mg/kg, 165 mg/kg, 170 mg/kg, 175 mg/kg, 180 mg/kg, 185 mg/kg, 190 mg/kg, 195 mg/kg, 200 mg/kg, and any fraction or integer in between any two of the preceding amounts.

In some embodiments, an nNOS inhibitor composition comprising one or more amino acid-based compounds, may be administered as a suitable dosage form, as described herein, to treat, ameliorate, and/or reduce the severity of neuroblastoma or one or more symptoms thereof in a neuroblastoma subject. In certain embodiments, an nNOS inhibitor composition comprising one or more amino acid-compounds that bind an active site of nNOS selected from the group consisting of Phenylalanine 589, Tryptophan 592, Tyrosine 593, Glutamate 597, Aspartate 602, Arginine 601, Methionine 341, Histidine 342, and Tyrosine711, may be administered as a suitable dosage form, as described herein, to treat, ameliorate, and/or reduce the severity of neuroblastoma or one or more symptoms thereof in a neuroblastoma subject. In some embodiments, an nNOS inhibitor composition comprising one or more amino acid-compounds that bind an active site of nNOS selected from the group consisting of Phenylalanine 589, Tryptophan 592, Tyrosine 593, Glutamate 597, Aspartate 602, Arginine 601, Methionine 341, Histidine 342, and Tyrosine711, may be administered to a neuroblastoma subject to treat, ameliorate, and/or reduce the severity of neuroblastoma or one or more symptoms thereof in the neuroblastoma subject: (i) via a route selected from the group consisting of buccally, inhalation, intraarterially, intraarticularly, intracardially, intracerebroventricularly, intradermally, intramedullary, intramuscularly, intraperitoneally, intrathecally, intravenously, intraventricularly, nasally, ophthalmically, orally, rectally, sublabially, sublingually, subcutaneously, topically, and transdermally; and/or (ii) with a frequency selected from the group consisting of four times daily, three times daily, twice daily, once daily, once every two days, once every three days, once every four days, once every five days, once every six days, once a week, once every eight days, once every nine days, once every ten days, once every two weeks, once every three weeks, and once a month; and/or (iii) at a dosage (based on subject weight) selected from the group consisting of 0.1 μg/kg, 0.5 μg/kg, 1 μg/kg, 1.5 μg/kg, 2.0 μg/kg, 2.5 μg/kg, 3.0 μg/kg, 3.5 μg/kg, 4.0 μg/kg, 4.5 μg/kg, 5.0 μg/kg, 10 μg/kg, 15 μg/kg, 20 μg/kg, 25 μg/kg, 30 μg/kg, 35 μg/kg, 40 μg/kg, 45 μg/kg, 50 μg/kg, 55 μg/kg, 60 μg/kg, 65 μg/kg, 70 μg/kg, 75 μg/kg, 80 μg/kg, 85 μg/kg, 90 μg/kg, 95 μg/kg, 100 μg/kg, 150 μg/kg, 200 μg/kg, 250 μg/kg, 300 μg/kg, 350 μg/kg, 400 μg/kg, 450 μg/kg, 500 μg/kg, 550 μg/kg, 600 μg/kg, 650 μg/kg, 700 μg/kg, 750 μg/kg, 800 μg/kg, 850 μg/kg, 900 μg/kg, 1 mg/kg, 1.5 mg/kg, 2.0 mg/kg, 2.5 mg/kg, 3 mg/kg, 3.5 mg/kg, 4.0 mg/kg, 4.5 mg/kg, 5 mg/kg, 5.5 mg/kg, 6 mg/kg, 6.5 mg/kg, 7 mg/kg, 7.5 mg/kg, 8 mg/kg, 8.5 mg/kg, 9 mg/kg, 9.5 mg/kg, 10 mg/kg 10.5 mg/kg, 11 mg/kg, 11.5 mg/kg, 12 mg/kg, 12.5 mg/kg, 13 mg/kg, 13.5 mg/kg, 14 mg/kg, 14.5 mg/kg, 15 mg/kg, 16 mg/kg, 17 mg/kg, 18 mg/kg, 19 mg/kg, 20 mg/kg, 21 mg/kg, 22 mg/kg, 23 mg/kg, 24 mg/kg, 25 mg/kg, 30 mg/kg, 35 mg/kg, 40 mg/kg, 45 mg/kg, 50 mg/kg, 55 mg/kg, 60 mg/kg, 65 mg/kg, 70 mg/kg, 75 mg/kg, 80 mg/kg, 85 mg/kg, 90 mg/kg, 95 mg/kg, 100 mg/kg, 105 mg/kg, 110 mg/kg, 115 mg/kg, 120 mg/kg, 125 mg/kg, 130 mg/kg, 135 mg/kg, 140 mg/kg, 145 mg/kg, 150 mg/kg, 155 mg/kg, 160 mg/kg, 165 mg/kg, 170 mg/kg, 175 mg/kg, 180 mg/kg, 185 mg/kg, 190 mg/kg, 195 mg/kg, 200 mg/kg, and any fraction or integer in between any two of the preceding amounts.

In some embodiments, a neuroblastoma subject, as described herein, may be a female child or a male child. In certain embodiments, a neuroblastoma subject, as described herein, may be a female adult or a male adult.

In some embodiments, a neuroblastoma subject, as described herein, may have an abnormal expression of one or more antigens compared to an equivalent subject that does not have neuroblastoma. As used herein, the term “equivalent subject,” refers to a subject of the same sex (male/female) and age range (child/adult) of the neuroblastoma subject. For purposes of disclosure, the term “antigen,” when used with respect to abnormal expression, may define one or more cluster of differentiation (“CD”) antigens, such as, for example, CD15, CD44, CD70, CD133, as well as any CD antigen that has an abnormal expression in neuroblastoma that would be readily envisaged by one skilled in the art. In certain embodiments, abnormal expression of an antigen may define an abnormal expression of CD15, an abnormal expression of CD44, an abnormal expression of CD70, an abnormal expression of CD133, an abnormal expression of a CD antigen that has an abnormal expression in neuroblastoma that would be readily envisaged by one skilled in the art, and any combination thereof. In some embodiments, an “abnormal expression,” can refer to a reduced expression of an antigen, as described herein. In certain embodiments, an “abnormal expression,” can refer to an overexpression of an antigen, as described herein. Whether “abnormal expression,” refers to a reduced expression of an antigen or an overexpression of an antigen would be immediately envisaged by the skilled artisan when viewing the term in the context of the particular antigen disclosed and neuroblastoma. For example, in the context of CD44 and neuroblastoma, one skilled in the art would immediately envisage that “abnormal expression” refers to an overexpression of CD44. Treatment, amelioration, and/or reduction in the severity of neuroblastoma or one or more symptoms thereof may be characterized by a restoration to a normal level of expression of one or more antigens from their abnormal state, as described herein. For example, in the context of CD44 and neuroblastoma, treatment, amelioration, and/or reduction in the severity of neuroblastoma or one or more symptoms thereof may be characterized by a reduced expression of CD44.

In certain embodiments, a neuroblastoma subject, as described herein, may have an abnormal expression of one or more proteins compared to an equivalent subject that does not have neuroblastoma. For purposes of disclosure, the term “protein,” when used with respect to abnormal expression, may define one or more proteins and/or enzymes, such as, for example, ALDH1A3, mTOR, p-mTOR, TSC-2, AKT, pAKT, RPS6, p-RPS6, Syp, VEGF, PIGF, as well as any protein or enzyme that has an abnormal expression in neuroblastoma that would be readily envisaged by one skilled in the art. In certain embodiments, abnormal expression of a protein may define an abnormal expression of ALDH1A3, an abnormal expression of mTOR, an abnormal expression of p-mTOR, an abnormal expression of TSC-2, an abnormal expression of AKT, an abnormal expression of pAKT, an abnormal expression of RPS6, an abnormal expression of p-RPS6, an abnormal expression of Syp, an abnormal expression of VEGF, an abnormal expression of PIGF, an abnormal expression of a protein or enzyme that has an abnormal expression in neuroblastoma that would be readily envisaged by one skilled in the art, and any combination thereof. In some embodiments, an “abnormal expression,” can refer to a reduced expression of a protein, as described herein. In certain embodiments, an “abnormal expression,” can refer to an overexpression of a protein, as described herein. Whether “abnormal expression,” refers to a reduced expression of a protein or an overexpression of a protein would be immediately envisaged by the skilled artisan when viewing the term in the context of the particular protein disclosed and neuroblastoma. For example, in the context of neuroblastoma, one skilled in the art would immediately envisage that “abnormal expression” refers to (i) an overexpression of mTOR, (ii) an overexpression of p-mTOR, (iii) a reduced expression of TSC-2, (iv) an overexpression of AKT, (v) an overexpression of pAKT, (vi) an overexpression of RPS6, (vii) an overexpression of p-RPS6, and/or (viii) an overexpression of Syp. Treatment, amelioration, and/or reduction in the severity of neuroblastoma or one or more symptoms thereof may be characterized by a restoration to a normal level of expression of one or more proteins from their abnormal state, as described herein. For example, in the context of neuroblastoma, treatment, amelioration, and/or reduction in the severity of neuroblastoma or one or more symptoms thereof may be characterized by a (i) a reduced expression of mTOR, (ii) a reduced expression of p-mTOR, (iii) an increased expression of TSC-2, (iv) a reduced expression of AKT, (v) a reduced expression of pAKT, (vi) a reduced expression of RPS6, (vii) a reduced expression of p-RPS6, and/or (viii) a reduced expression of Syp.

In certain embodiments, a neuroblastoma subject, as described herein, may have an increased expression of one or more NOS isoforms compared to an equivalent subject that does not have neuroblastoma. For purposes of disclosure, the term “NOS isoform,” refers to nNOS, iNOS, and/or eNOS. In certain embodiments, a neuroblastoma subject, as described herein, may have an increased expression of nNOS compared to an equivalent subject that does not have neuroblastoma. In some embodiments, a neuroblastoma subject, as described herein, may have an increased expression of iNOS compared to an equivalent subject that does not have neuroblastoma. In certain embodiments, a neuroblastoma subject, as described herein, may have an increased expression of eNOS compared to an equivalent subject that does not have neuroblastoma. In some embodiments, a neuroblastoma subject, as described herein, may have an increased expression of nNOS and iNOS compared to an equivalent subject that does not have neuroblastoma. In certain embodiments, a neuroblastoma subject, as described herein, may have an increased expression of nNOS and eNOS compared to an equivalent subject that does not have neuroblastoma. In some embodiments, a neuroblastoma subject, as described herein, may have an increased expression of iNOS and eNOS compared to an equivalent subject that does not have neuroblastoma. In certain embodiments, a neuroblastoma subject, as described herein, may have an increased expression of nNOS, iNOS, and eNOS compared to an equivalent subject that does not have neuroblastoma. Whether a “NOS isoform,” has an increased expression would be immediately envisaged by the skilled artisan when viewing the term in the context of the particular NOS isoform disclosed and neuroblastoma. For example, in the context of neuroblastoma, one skilled in the art would immediately envisage that nNOS and/or eNOS have an increased expression. Treatment, amelioration, and/or reduction in the severity of neuroblastoma or one or more symptoms thereof may be characterized by a reduced expression of one or more NOS isoforms from their increased state, as described herein. For example, in the context of neuroblastoma, treatment, amelioration, and/or reduction in the severity of neuroblastoma or one or more symptoms thereof may be characterized by a reduced expression of nNOS and/or eNOS. For purposes of disclosure, reduction/inhibition of the expression of one or more NOS isoforms may be characterized by a reduction in expression of one or more NOS isoforms by about at least 10%, about at least 20%, about at least 30%, or about at least 40%. In some embodiments, reduction/inhibition of the expression of one or more NOS isoforms may further be characterized by a lack reduction of other, non-implicated NOS isoforms, such as, for example, no more than about 10%, no more than about 5%, no more than about 2%, or no more than about 1% reduction/inhibition of the expression of non-implicated NOS isoforms. As used herein, the term “non-implicated NOS isoforms,” refers to NOS isoforms that do not have an increased expression in neuroblastoma. By way of example, in the context of neuroblastoma, if both nNOS and eNOS have an increased expression, iNOS would define a “non-implicated” NOS isoform. Whether a NOS isoform is a “non-implicated NOS isoform,” would be immediately envisaged by the skilled artisan when viewing the term in the context of the particular NOS isoform disclosed and neuroblastoma.

In some embodiments, a neuroblastoma subject, as described herein, may have an elevated level of one or more NO-indicators compared to an equivalent subject that does not have neuroblastoma. As used herein, the term “NO-indicator,” can refer to 3-nitrotyrosine (3-Ntyr), NADPH-diaphorase, total nitrite concentration, total nitrate concentration, total nitrite and nitrate concentration, as well as any NO-indicator that has an elevated level in neuroblastoma that would be readily envisaged by one skilled in the art. For example, in the context of neuroblastoma, one skilled in the art would immediately envisage that 3-Ntyr, NADPH-diaphorase, and/or total nitrite concentration may be present at an elevated level. Treatment, amelioration, and/or reduction in the severity of neuroblastoma or one or more symptoms thereof may be characterized by a reduction in the level of one or more NO-indicators. For example, in the context of neuroblastoma, treatment, amelioration, and/or reduction in the severity of neuroblastoma or one or more symptoms thereof may be characterized by a reduction in the level of 3-Ntyr, NADPH-diaphorase, and/or total nitrite concentration.

In certain embodiments, a NOS inhibitor composition, as described herein, may be administered as a suitable dosage form, as described herein, to treat, ameliorate, and/or reduce the severity of neuroblastoma or one or more symptoms thereof, as an alternative to a conventional therapy for treating, ameliorating, and/or reducing the severity of neuroblastoma or one or more symptoms thereof. For purposes of disclosure, a “conventional therapy,” as described herein, may refer to a treatment that would be recommended by one skilled in the art to treat neuroblastoma, and may define one or more of radiation, chemotherapy, immunotherapy, cell-based therapy, gene therapy, and the like—one skilled in the art would immediately envisage a “conventional therapy” based on the particular neuroblastoma disclosed, as well as characteristics of a neuroblastoma subject, such as, for example, the subject's sex, age, stage of the neuroblastoma, genotype of the neuroblastoma, and the like. Treatment, amelioration, and/or reduction in the severity of neuroblastoma or one or more symptoms thereof may be characterized by an improved outcome compared to a conventional therapy, as described herein. For purposes of the disclosure, an “improved outcome” may define any one of the following: (i) a larger reduction in tumor volume and/or mass compared to a subject administered an equivalent dose of a conventional therapy; (ii) an improvement in the expected rate of survival of a subject, at, for example, 1 month, 3 months, 6 months, 9 months, 1 year, 2 years, 3 years, 5 years, 10 years, or more, compared to the expected rate of survival of a subject administered an equivalent dose of a conventional therapy; (iii) a reduction in the prevalence of side effects resulting from the NOS inhibitor composition compared to a subject administered an equivalent dose of a conventional therapy; (iv) a reduction in the severity of side effects resulting from the NOS inhibitor composition compared to a subject administered an equivalent dose of a conventional therapy; (v) a reduction in the dosage required to achieve an equivalent reduction in tumor volume compared to a subject administered a conventional therapy; (vi) a reduction in the dosage required to achieve an equivalent expected rate of survival of a subject compared to a subject administered a conventional therapy; (vii) a reduction in the likelihood of a recurrence of the neuroblastoma occurring in a subject compared to a subject administered an equivalent dose of a conventional therapy; (viii) a reduction in the likelihood of a metastasis occurring in a subject compared to a subject administered an equivalent dose of a conventional therapy; (ix) a reduction in the time required to be at a medical facility for treatment compared to a subject administered an equivalent dose of a conventional therapy; (x) a reduction in the cost associated with treatment compared to a subject administered an equivalent dose of a conventional therapy; as well as any other improved outcome not expressly recited that would be readily envisaged by one skilled in the art.

In some embodiments, a NOS inhibitor composition, as described herein, may be administered as a suitable dosage form, as described herein, to treat, ameliorate, and/or reduce the severity of neuroblastoma or one or more symptoms thereof, wherein the NOS inhibitor composition comprises a first chemical NOS inhibitor and a second chemical NOS inhibitor. In certain embodiments, the first chemical NOS inhibitor may be administered concurrently with the second chemical NOS inhibitor. In some embodiments, the first chemical NOS inhibitor may be administered prior to the second chemical NOS inhibitor. In certain embodiments, the first chemical NOS inhibitor may be administered subsequently to the second chemical NOS inhibitor. Treatment, amelioration, and/or reduction in the severity of neuroblastoma or one or more symptoms thereof may be characterized by an improved outcome compared an alternative treatment. As used herein, the term “alternative treatment,” may define one or more of a conventional therapy, as described herein, a NOS inhibitor composition comprising a single chemical NOS inhibitor, as described herein, and a NOS inhibitor composition comprising a single biological NOS inhibitor, as described herein. For purposes of the disclosure, an “improved outcome” may define any one of the following: (i) a larger reduction in tumor volume and/or mass compared an alternative treatment; (ii) an improvement in the expected rate of survival of a subject, at, for example, 1 month, 3 months, 6 months, 9 months, 1 year, 2 years, 3 years, 5 years, 10 years, or more, compared to the expected rate of survival of a subject administered an alternative treatment; (iii) a reduction in the prevalence of side effects resulting from the NOS inhibitor composition comprising at least two chemical NOS inhibitors compared to a subject administered an alternative treatment; (iv) a reduction in the severity of side effects resulting from the NOS inhibitor composition comprising at least two chemical NOS inhibitors compared to a subject administered an alternative treatment; (v) a reduction in the dosage required to achieve an equivalent reduction in tumor volume compared to a subject administered an alternative treatment; (vi) a reduction in the dosage required to achieve an equivalent expected rate of survival of a subject compared to a subject administered an alternative treatment; (vii) a reduction in the likelihood of a recurrence of the neuroblastoma in a subject compared to a subject administered an alternative treatment; (viii) a reduction in the likelihood of a metastasis occurring in a subject compared to a subject administered an alternative treatment; (ix) a reduction in the time required to be at a medical facility for treatment compared to a subject administered an alternative treatment; (x) a reduction in the cost associated with treatment compared to a subject administered an alternative treatment; as well as any other improved outcome not expressly recited that would be readily envisaged by one skilled in the art.

In certain embodiments, a NOS inhibitor composition, as described herein, may be used to treat, ameliorate, and/or reduce the severity of neuroblastoma or one or more symptoms thereof, wherein the NOS inhibitor composition comprises at least one chemical NOS inhibitor and at least one biological NOS inhibitor. In some embodiments, the at least one chemical NOS inhibitor may be administered concurrently with the at least one biological NOS inhibitor. In certain embodiments, the at least one chemical NOS inhibitor may be administered prior to the at least one biological NOS inhibitor. In some embodiments, the at least one chemical NOS inhibitor may be administered subsequently to the at least one biological NOS inhibitor. Treatment, amelioration, and/or reduction in the severity of neuroblastoma or one or more symptoms thereof may be characterized by an improved outcome compared an alternative treatment. As used herein, the term “alternative treatment,” may define one or more of a conventional therapy, as described herein, a NOS inhibitor composition comprising a single chemical NOS inhibitor, as described herein, and a NOS inhibitor composition comprising a single biological NOS inhibitor, as described herein. For purposes of the disclosure, an “improved outcome” may define any one of the following: (i) a larger reduction in tumor volume and/or mass compared an alternative treatment; (ii) an improvement in the expected rate of survival of a subject, at, for example, 1 month, 3 months, 6 months, 9 months, 1 year, 2 years, 3 years, 5 years, 10 years, or more, compared to the expected rate of survival of a subject administered an alternative treatment; (iii) a reduction in the prevalence of side effects resulting from the NOS inhibitor composition comprising at least one chemical NOS inhibitor and at least one biological NOS inhibitor compared to a subject administered an alternative treatment; (iv) a reduction in the severity of side effects resulting from the NOS inhibitor composition comprising at least one chemical NOS inhibitor and at least one biological NOS inhibitor compared to a subject administered an alternative treatment; (v) a reduction in the dosage required to achieve an equivalent reduction in tumor volume compared to a subject administered an alternative treatment; (vi) a reduction in the dosage required to achieve an equivalent expected rate of survival of a subject compared to a subject administered an alternative treatment; (vii) a reduction in the likelihood of a recurrence of the neuroblastoma in a subject compared to a subject administered an alternative treatment; (viii) a reduction in the likelihood of a metastasis occurring in a subject compared to a subject administered an alternative treatment; (ix) a reduction in the time required to be at a medical facility for treatment compared to a subject administered an alternative treatment; (x) a reduction in the cost associated with treatment compared to a subject administered an alternative treatment; as well as any other improved outcome not expressly recited that would be readily envisaged by one skilled in the art.

In some embodiments, a NOS inhibitor composition, as described herein, may be combined with a conventional therapy, as described herein, to treat, ameliorate, and/or reduce the severity of neuroblastoma or one or more symptoms thereof. In certain embodiments, the NOS inhibitor composition may be administered concurrently with the conventional therapy. In some embodiments, the NOS inhibitor composition may be administered prior to the conventional therapy. In certain embodiments, the NOS inhibitor composition may be administered subsequently to the conventional therapy. Treatment, amelioration, and/or reduction in the severity of neuroblastoma or one or more symptoms thereof may be characterized by an improved outcome compared an alternative treatment. As used herein, the term “alternative treatment,” may define one or more of a conventional therapy, as described herein, a NOS inhibitor composition comprising a single chemical NOS inhibitor, as described herein, and a NOS inhibitor composition comprising a single biological NOS inhibitor, as described herein. For purposes of the disclosure, an “improved outcome” may define any one of the following: (i) a larger reduction in tumor volume and/or mass compared an alternative treatment; (ii) an improvement in the expected rate of survival of a subject, at, for example, 1 month, 3 months, 6 months, 9 months, 1 year, 2 years, 3 years, 5 years, 10 years, or more, compared to the expected rate of survival of a subject administered an alternative treatment; (iii) a reduction in the prevalence of side effects resulting from the NOS inhibitor composition and conventional therapy compared to a subject administered an alternative treatment; (iv) a reduction in the severity of side effects resulting from the NOS inhibitor composition and conventional therapy compared to a subject administered an alternative treatment; (v) a reduction in the dosage required to achieve an equivalent reduction in tumor volume compared to a subject administered an alternative treatment; (vi) a reduction in the dosage required to achieve an equivalent expected rate of survival of a subject compared to a subject administered an alternative treatment; (vii) a reduction in the likelihood of a recurrence of the neuroblastoma in a subject compared to a subject administered an alternative treatment; (viii) a reduction in the likelihood of a metastasis occurring in a subject compared to a subject administered an alternative treatment; (ix) a reduction in the time required to be at a medical facility for treatment compared to a subject administered an alternative treatment; (x) a reduction in the cost associated with treatment compared to a subject administered an alternative treatment; as well as any other improved outcome not expressly recited that would be readily envisaged by one skilled in the art.

In some embodiments, a “conventional therapy,” as that term is used with respect to combined administration with a NOS inhibitor composition, may refer to a conventional chemotherapy. For purposes of the disclosure, a “conventional chemotherapy,” as described herein may be a compound selected from the group consisting of abemaciclib, abiraterone acetate, acalabrutinib, adagrasib, afatinib dimaleate, albumin-bound paclitaxel, albumin-bound rapamycin (Sirolimus Protein-Bound Particles), alectinib, alpelisib, amifostine, aminolevulinic acid hydrochloride, anastrozole, apalutamide, aprepitant, arsenic trioxide, asciminib hydrochloride, avapritinib, axitinib, azacytidine, belinostat, belzutifan, bendamustine hydrochloride, bexarotene, bicalutamide, binimetinib, bleomycin, bleomycin sulfate, bortezomib, bosutinib, brigatinib, busulfan, cabazitaxel, cabozantinib-S-malate, capecitabine, capmatinib hydrochloride, carboplatin, carfilzomib, carmustine, cedazuridine, ceritinib, chlorambucil, cisplatin, cladribine, clofarabine, cobimetinib fumarate, copanlisib hydrochloride, crizotinib, cyclophosphamide, cytarabine, dabrafenib mesylate, dacarbazine, dacomitinib, dactinomycin, darolutamide, dasatinib, daunorubicin hydrochloride, decitabine, defibrotide sodium, degarelix, dexamethasone, dexrazoxane hydrochloride, docetaxel, doxorubicin hydrochloride, duvelisib, elacestrant dihydrochloride, eltrombopag olamine, enasidenib mesylate, encorafenib, entrectinib, enzalutamide, epirubicin hydrochloride, erdafitinib, eribulin mesylate, erlotinib hydrochloride, etoposide, etoposide phosphate, everolimus, exemestane, fedratinib hydrochloride, fludarabine phosphate, fluorouracil, flutamide, folinic acid, fostamatinib disodium, fulvestrant, futibatinib, gefitinib, gemcitabine hydrochloride, gilteritinib fumarate, glasdegib maleate, goserelin acetate, granisetron, granisetron hydrochloride, hydroxyurea, ibrutinib, idarubicin hydrochloride, idelalisib, ifosfamide, imatinib mesylate, imiquimod, infigratinib phosphate, iobenguane I-131, irinotecan hydrochloride, ivosidenib, ixabepilone, ixazomib citrate, lanreotide acetate, lapatinib ditosylate, larotrectinib sulfate, lenalidomide, lenvatinib mesylate, letrozole, leucovorin calcium, leuprolide acetate, liposomal cytarabine, liposomal doxorubicin hydrochloride, liposomal irinotecan hydrochloride, lomustine, lorlatinib, lurbinectedin, Lu-177 dotatate, Lu-177 vipivotide tetraxetan, mechlorethamine hydrochloride, megestrol acetate, melphalan, melphalan hydrochloride, mercaptopurine, mesna, methotrexate, methotrexate sodium, methylnaltrexone bromide, midostaurin, mitomycin, mitoxantrone hydrochloride, mobocertinib succinate, nelarabine, neratinib maleate, netupitant hydrochloride, nilutamide, nilotinib, niraparib tosylate monohydrate, olaparib, olutasidenib, omacetaxine mepesuccinate, ondansetron hydrochloride, osimertinib mesylate, oxaliplatin, paclitaxel, pacritinib citrate, palbociclib, palonosetron hydrochloride, pamidronate disodium, pazopanib hydrochloride, pemetrexed disodium, pemigatinib, pexidartinib hydrochloride, pirtobrutinib, plerixafor, pomalidomide, ponatinib hydrochloride, pralatrexate, pralsetinib, prednisone, procarbazine hydrochloride, propranolol hydrochloride, Ra-223 dichloride, raloxifene hydrochloride, regorafenib, relugolix, ribociclib, ripretinib, rolapitant hydrochloride, romidepsin, rucaparib camsylate, ruxolitinib phosphate, selinexor, selpercatinib, selumetinib sulfate, sonidegib, sorafenib tosylate, sotorasib, sunitinib malate, talazoparib tosylate, talc, tamoxifen citrate, tazemetostat hydrobromide, temozolomide, temsirolimus, tepotinib hydrochloride, thalidomide, thioguanine, thiotepa, tipiracil hydrochloride, tivozanib hydrochloride, topotecan hydrochloride, toremifene, trabectedin, trametinib dimethyl sulfoxide, tretinoin, trifluridine hydrochloride, tucatinib, uridine triacetate, valrubicin, vandetanib, vemurafenib, venetoclax, vinblastine sulfate, vincristine sulfate, vinorelbine tartrate, vismodegib, vorinostat, zanubrutinib, zoledronic acid, and any combination thereof. One skilled in the art would immediately envisage an appropriate “conventional chemotherapy” based on the particular neuroblastoma disclosed, as well as characteristics of a neuroblastoma subject, such as, for example, the subject's sex, age, stage of the neuroblastoma, genotype of the neuroblastoma, and the like.

In certain embodiments, a “conventional therapy,” as that term is used with respect to combined administration with a NOS inhibitor composition, may refer to a conventional immunotherapy. For purposes of the disclosure, a “conventional immunotherapy,” as described herein may be an immunotherapy, cell-based therapy, and/or gene-based therapy selected from the group consisting of ado-trastuzumab emtansine, aldesleukin, alemtuzumab, amivantamab-vmjw, asparaginase Erwinia chrysanthemi, asparaginase Erwinia chrysanthemi (recombinant)-rywn, atezolizumab, avelumab, axicabtagene ciloleucel, bevacizumab, blinatumomab, brentuximab vedotin, brexucabtagene autoleucel, calaspargase pegol-mknl, camrelizumab, caplacizumab-yhdp, cemiplimab-rwlc, cetuximab, ciltacabtagene autoleucel, daratumumab, daratumumab and hyaluronidase-fihj, darbepoetin alfa, denileukin diftitox, denosumab, dinutuximab, dostarlimab-gxly, durvalumab, elotuzumab, emapalumab-lzsg, enfortumab vedotin-ejfv, epoetin alfa, fam-trastuzumab deruxtecan-nxki, filgrastim, gemtuzumab ozogamicin, glucarpidase, ibritumomab tiuxetan, idecabtagene vicleucel, inotuzumab ozogamicin, ipilimumab, isatuximab-irfc, lisocabtagene maraleucel, loncastuximab tesirine-lpyl, luspatercept-aamt, margetuximab-cmkb, mirvetuximab soravtansine-gynx, mogamulizumab-kpkc, mosunetuzumab-axgb, moxetumomab pasudotox-tdfk, nadofaragene firadenovec-vncg, naxitamab-gqgk, necitumumab, nivolumab, nivolumab and relatlimab-rmbw, obinutuzumab, ofatumumab, palifermin, panitumumab, pegaspargase, pegfilgrastim, pembrolizumab, pertuzumab, pertuzumab, trastuzumab, and hyaluronidase-zzxf, polatuzumab vedotin-piiq, ramucirumab, rasburicase, ravulizumab-cwvz, recombinant HPV bivalent vaccine, recombinant HPV nonavalent vaccine, recombinant HPV quadrivalent vaccine, recombinant interferon alfa-2b, rituximab, rituximab and hyaluronidase human, romiplostim, ropeginterferon alfa-2b-njft, sacituzumab govitecan-hziy, siltuximab, sintilimab, sipuleucel-T, tafasitamab-cxix, tagraxofusp-erzs, talimogene laherparepvec, tebentafusp-tebn, teclistamab-cqyv, tisagenlecleucel, tislelizumab, tisotumab vedotin-tftv, tocilizumab, toripalimab, trastuzumab, trastuzumab and hyaluronidase-oysk, tremelimumab-actl, ziv-aflibercept, and any combination thereof. One skilled in the art would immediately envisage an appropriate “conventional immunotherapy” based on the particular neuroblastoma disclosed, as well as characteristics of a neuroblastoma subject, such as, for example, the subject's sex, age, stage of the neuroblastoma, genotype of the neuroblastoma, and the like.

In certain embodiments, a NOS inhibitor composition, as described herein, may be administered as a suitable dosage form, as described herein, prior to, or alternatively, following a surgery to remove or resect neuroblastoma.

In some embodiments, a NOS inhibitor composition, as described herein, may be administered as a suitable dosage form, as described herein, to restore and/or maintain healthy and/or optimal expression levels of one or more antigens, as described herein, such as, for example, CD15, CD44, CD70, CD133, as well as any CD antigen that has an abnormal expression in neuroblastoma that would be readily envisaged by one skilled in the art, in a subject. In certain embodiments, the subject may have neuroblastoma or one or more symptoms thereof. As used herein, the terms “healthy” and “optimal” refer to expression levels of one or more antigens that would be typical of an equivalent subject that does not have neuroblastoma.

In certain embodiments, a NOS inhibitor composition, as described herein, may be administered as a suitable dosage form, as described herein, to restore and/or maintain healthy and/or optimal expression levels of one or more proteins, as described herein, such as, for example, ALDH1A3, mTOR, p-mTOR, TSC-2, AKT, pAKT, RPS6, p-RPS6, Syp, VEGF, PIGF, as well as any protein or enzyme that has an abnormal expression in neuroblastoma that would be readily envisaged by one skilled in the art, in a subject. In certain embodiments, the subject may have neuroblastoma or one or more symptoms thereof. As used herein, the terms “healthy” and “optimal” refer to expression levels of one or more proteins that would be typical of an equivalent subject that does not have neuroblastoma.

In some embodiments, a NOS inhibitor composition, as described herein, may be administered as a suitable dosage form, as described herein, to restore and/or maintain healthy and/or optimal expression levels of one or more NOS isoforms, as described herein, such as, for example, nNOS, iNOS, and eNOS, in a subject. In certain embodiments, the subject may have neuroblastoma or one or more symptoms thereof. As used herein, the terms “healthy” and “optimal” refer to expression levels of one or more NOS isoforms that would be typical of an equivalent subject that does not have neuroblastoma.

In certain embodiments, a NOS inhibitor composition, as described herein, may be administered as a suitable dosage form, as described herein, to restore and/or maintain healthy and/or optimal levels of one or more NO-indicators, as described herein, such as, for example, 3-Ntyr, NADPH-diaphorase, total nitrite concentration, total nitrate concentration, total nitrite and nitrate concentration, as well as any NO-indicator that has an elevated level in neuroblastoma that would be readily envisaged by one skilled in the art, in a subject. In certain embodiments, the subject may have neuroblastoma or one or more symptoms thereof. As used herein, the terms “healthy” and “optimal” refer to levels of one or more NO-indicators that would be typical of an equivalent subject that does not have neuroblastoma.

In certain embodiments, a NOS inhibitor composition, as described herein, may be administered as a suitable dosage form, as described herein, to treat, ameliorate, and/or reduce the severity of a pancreatic cancer or one or more symptoms thereof in a subject (“pancreatic cancer subject”). As used herein, the term “pancreatic cancer” refers to a cancer that occurs and/or originates in a portion of the pancreas including, but not limited to the body, duct, head, neck, and tail.

In certain embodiments, a NOS inhibitor composition, as described herein, may be administered as a suitable dosage form, as described herein, to treat, ameliorate, and/or reduce the severity of a pancreatic cancer or one or more symptoms thereof in a pancreatic cancer subject.

In some embodiments, a NOS inhibitor composition comprising 7-NI and/or a salt thereof may be administered as a suitable dosage form, as described herein, to treat, ameliorate, and/or reduce the severity of pancreatic cancer or one or more symptoms thereof in a pancreatic cancer subject. In certain embodiments, an nNOS inhibitor composition comprising 7-NI and/or a salt thereof may be administered to a pancreatic cancer subject to treat, ameliorate, and/or reduce the severity of pancreatic cancer or one or more symptoms thereof in the pancreatic cancer subject: (i) via a route selected from the group consisting of buccally, inhalation, intraarterially, intraarticularly, intracardially, intracerebroventricularly, intradermally, intramedullary, intramuscularly, intraperitoneally, intrathecally, intravenously, intraventricularly, nasally, ophthalmically, orally, rectally, sublabially, sublingually, subcutaneously, topically, and transdermally; and/or (ii) with a frequency selected from the group consisting of four times daily, three times daily, twice daily, once daily, once every two days, once every three days, once every four days, once every five days, once every six days, once a week, once every eight days, once every nine days, once every ten days, once every two weeks, once every three weeks, and once a month; and/or (iii) at a dosage (based on subject weight) selected from the group consisting of 0.1 μg/kg, 0.5 μg/kg, 1 μg/kg, 1.5 μg/kg, 2.0 μg/kg, 2.5 μg/kg, 3.0 μg/kg, 3.5 μg/kg, 4.0 μg/kg, 4.5 μg/kg, 5.0 μg/kg, 10 μg/kg, 15 μg/kg, 20 μg/kg, 25 μg/kg, 30 μg/kg, 35 μg/kg, 40 μg/kg, 45 μg/kg, 50 μg/kg, 55 μg/kg, 60 μg/kg, 65 μg/kg, 70 μg/kg, 75 μg/kg, 80 μg/kg, 85 μg/kg, 90 μg/kg, 95 μg/kg, 100 μg/kg, 150 μg/kg, 200 μg/kg, 250 μg/kg, 300 μg/kg, 350 μg/kg, 400 μg/kg, 450 μg/kg, 500 μg/kg, 550 μg/kg, 600 μg/kg, 650 μg/kg, 700 μg/kg, 750 μg/kg, 800 μg/kg, 850 μg/kg, 900 μg/kg, 1 mg/kg, 1.5 mg/kg, 2.0 mg/kg, 2.5 mg/kg, 3 mg/kg, 3.5 mg/kg, 4.0 mg/kg, 4.5 mg/kg, 5 mg/kg, 5.5 mg/kg, 6 mg/kg, 6.5 mg/kg, 7 mg/kg, 7.5 mg/kg, 8 mg/kg, 8.5 mg/kg, 9 mg/kg, 9.5 mg/kg, 10 mg/kg 10.5 mg/kg, 11 mg/kg, 11.5 mg/kg, 12 mg/kg, 12.5 mg/kg, 13 mg/kg, 13.5 mg/kg, 14 mg/kg, 14.5 mg/kg, 15 mg/kg, 16 mg/kg, 17 mg/kg, 18 mg/kg, 19 mg/kg, 20 mg/kg, 21 mg/kg, 22 mg/kg, 23 mg/kg, 24 mg/kg, 25 mg/kg, 30 mg/kg, 35 mg/kg, 40 mg/kg, 45 mg/kg, 50 mg/kg, 55 mg/kg, 60 mg/kg, 65 mg/kg, 70 mg/kg, 75 mg/kg, 80 mg/kg, 85 mg/kg, 90 mg/kg, 95 mg/kg, 100 mg/kg, 105 mg/kg, 110 mg/kg, 115 mg/kg, 120 mg/kg, 125 mg/kg, 130 mg/kg, 135 mg/kg, 140 mg/kg, 145 mg/kg, 150 mg/kg, 155 mg/kg, 160 mg/kg, 165 mg/kg, 170 mg/kg, 175 mg/kg, 180 mg/kg, 185 mg/kg, 190 mg/kg, 195 mg/kg, 200 mg/kg, and any fraction or integer in between any two of the preceding amounts.

In certain embodiments, a NOS inhibitor composition comprising 3-Br-7-NI and/or a salt thereof may be administered as a suitable dosage form, as described herein, to treat, ameliorate, and/or reduce the severity of pancreatic cancer or one or more symptoms thereof in a pancreatic cancer subject. In some embodiments, an nNOS inhibitor composition comprising 3-Br-7-NI and/or a salt thereof may be administered to a pancreatic cancer subject to treat, ameliorate, and/or reduce the severity of pancreatic cancer or one or more symptoms thereof in the pancreatic cancer subject: (i) via a route selected from the group consisting of buccally, inhalation, intraarterially, intraarticularly, intracardially, intracerebroventricularly, intradermally, intramedullary, intramuscularly, intraperitoneally, intrathecally, intravenously, intraventricularly, nasally, ophthalmically, orally, rectally, sublabially, sublingually, subcutaneously, topically, and transdermally; and/or (ii) with a frequency selected from the group consisting of four times daily, three times daily, twice daily, once daily, once every two days, once every three days, once every four days, once every five days, once every six days, once a week, once every eight days, once every nine days, once every ten days, once every two weeks, once every three weeks, and once a month; and/or (iii) at a dosage (based on subject weight) selected from the group consisting of 0.1 μg/kg, 0.5 μg/kg, 1 μg/kg, 1.5 μg/kg, 2.0 μg/kg, 2.5 μg/kg, 3.0 μg/kg, 3.5 μg/kg, 4.0 μg/kg, 4.5 μg/kg, 5.0 μg/kg, 10 μg/kg, 15 μg/kg, 20 μg/kg, 25 μg/kg, 30 μg/kg, 35 μg/kg, 40 μg/kg, 45 μg/kg, 50 μg/kg, 55 μg/kg, 60 μg/kg, 65 μg/kg, 70 μg/kg, 75 μg/kg, 80 μg/kg, 85 μg/kg, 90 μg/kg, 95 μg/kg, 100 μg/kg, 150 μg/kg, 200 μg/kg, 250 μg/kg, 300 μg/kg, 350 μg/kg, 400 μg/kg, 450 μg/kg, 500 μg/kg, 550 μg/kg, 600 μg/kg, 650 μg/kg, 700 μg/kg, 750 μg/kg, 800 μg/kg, 850 μg/kg, 900 μg/kg, 1 mg/kg, 1.5 mg/kg, 2.0 mg/kg, 2.5 mg/kg, 3 mg/kg, 3.5 mg/kg, 4.0 mg/kg, 4.5 mg/kg, 5 mg/kg, 5.5 mg/kg, 6 mg/kg, 6.5 mg/kg, 7 mg/kg, 7.5 mg/kg, 8 mg/kg, 8.5 mg/kg, 9 mg/kg, 9.5 mg/kg, 10 mg/kg 10.5 mg/kg, 11 mg/kg, 11.5 mg/kg, 12 mg/kg, 12.5 mg/kg, 13 mg/kg, 13.5 mg/kg, 14 mg/kg, 14.5 mg/kg, 15 mg/kg, 16 mg/kg, 17 mg/kg, 18 mg/kg, 19 mg/kg, 20 mg/kg, 21 mg/kg, 22 mg/kg, 23 mg/kg, 24 mg/kg, 25 mg/kg, 30 mg/kg, 35 mg/kg, 40 mg/kg, 45 mg/kg, 50 mg/kg, 55 mg/kg, 60 mg/kg, 65 mg/kg, 70 mg/kg, 75 mg/kg, 80 mg/kg, 85 mg/kg, 90 mg/kg, 95 mg/kg, 100 mg/kg, 105 mg/kg, 110 mg/kg, 115 mg/kg, 120 mg/kg, 125 mg/kg, 130 mg/kg, 135 mg/kg, 140 mg/kg, 145 mg/kg, 150 mg/kg, 155 mg/kg, 160 mg/kg, 165 mg/kg, 170 mg/kg, 175 mg/kg, 180 mg/kg, 185 mg/kg, 190 mg/kg, 195 mg/kg, 200 mg/kg, and any fraction or integer in between any two of the preceding amounts.

In some embodiments, a NOS inhibitor composition comprising L-NPA and/or a salt thereof, such as, for example, L-NPA hydrochloride, may be administered as a suitable dosage form, as described herein, to treat, ameliorate, and/or reduce the severity of pancreatic cancer or one or more symptoms thereof in a pancreatic cancer subject. In certain embodiments, an nNOS inhibitor composition comprising L-NPA and/or a salt thereof, such as, for example, L-NPA hydrochloride may be administered to a pancreatic cancer subject to treat, ameliorate, and/or reduce the severity of pancreatic cancer or one or more symptoms thereof in the pancreatic cancer subject: (i) via a route selected from the group consisting of buccally, inhalation, intraarterially, intraarticularly, intracardially, intracerebroventricularly, intradermally, intramedullary, intramuscularly, intraperitoneally, intrathecally, intravenously, intraventricularly, nasally, ophthalmically, orally, rectally, sublabially, sublingually, subcutaneously, topically, and transdermally; and/or (ii) with a frequency selected from the group consisting of four times daily, three times daily, twice daily, once daily, once every two days, once every three days, once every four days, once every five days, once every six days, once a week, once every eight days, once every nine days, once every ten days, once every two weeks, once every three weeks, and once a month; and/or (iii) at a dosage (based on subject weight) selected from the group consisting of 0.1 μg/kg, 0.5 μg/kg, 1 μg/kg, 1.5 μg/kg, 2.0 μg/kg, 2.5 μg/kg, 3.0 μg/kg, 3.5 μg/kg, 4.0 μg/kg, 4.5 μg/kg, 5.0 μg/kg, 10 μg/kg, 15 μg/kg, 20 μg/kg, 25 μg/kg, 30 μg/kg, 35 μg/kg, 40 μg/kg, 45 μg/kg, 50 μg/kg, 55 μg/kg, 60 μg/kg, 65 μg/kg, 70 μg/kg, 75 μg/kg, 80 μg/kg, 85 μg/kg, 90 μg/kg, 95 μg/kg, 100 μg/kg, 150 μg/kg, 200 μg/kg, 250 μg/kg, 300 μg/kg, 350 μg/kg, 400 μg/kg, 450 μg/kg, 500 μg/kg, 550 μg/kg, 600 μg/kg, 650 μg/kg, 700 μg/kg, 750 μg/kg, 800 μg/kg, 850 μg/kg, 900 μg/kg, 1 mg/kg, 1.5 mg/kg, 2.0 mg/kg, 2.5 mg/kg, 3 mg/kg, 3.5 mg/kg, 4.0 mg/kg, 4.5 mg/kg, 5 mg/kg, 5.5 mg/kg, 6 mg/kg, 6.5 mg/kg, 7 mg/kg, 7.5 mg/kg, 8 mg/kg, 8.5 mg/kg, 9 mg/kg, 9.5 mg/kg, 10 mg/kg 10.5 mg/kg, 11 mg/kg, 11.5 mg/kg, 12 mg/kg, 12.5 mg/kg, 13 mg/kg, 13.5 mg/kg, 14 mg/kg, 14.5 mg/kg, 15 mg/kg, 16 mg/kg, 17 mg/kg, 18 mg/kg, 19 mg/kg, 20 mg/kg, 21 mg/kg, 22 mg/kg, 23 mg/kg, 24 mg/kg, 25 mg/kg, 30 mg/kg, 35 mg/kg, 40 mg/kg, 45 mg/kg, 50 mg/kg, 55 mg/kg, 60 mg/kg, 65 mg/kg, 70 mg/kg, 75 mg/kg, 80 mg/kg, 85 mg/kg, 90 mg/kg, 95 mg/kg, 100 mg/kg, 105 mg/kg, 110 mg/kg, 115 mg/kg, 120 mg/kg, 125 mg/kg, 130 mg/kg, 135 mg/kg, 140 mg/kg, 145 mg/kg, 150 mg/kg, 155 mg/kg, 160 mg/kg, 165 mg/kg, 170 mg/kg, 175 mg/kg, 180 mg/kg, 185 mg/kg, 190 mg/kg, 195 mg/kg, 200 mg/kg, and any fraction or integer in between any two of the preceding amounts.

In certain embodiments, a NOS inhibitor composition comprising L-NIO and/or a salt thereof, such as, for example, L-NIO dihydrochloride, may be administered as a suitable dosage form, as described herein, to treat, ameliorate, and/or reduce the severity of pancreatic cancer or one or more symptoms thereof in a pancreatic cancer subject. In some embodiments, an nNOS inhibitor composition comprising L-NIO and/or a salt thereof, such as, for example, L-NIO dihydrochloride, may be administered to a pancreatic cancer subject to treat, ameliorate, and/or reduce the severity of pancreatic cancer or one or more symptoms thereof in the pancreatic cancer subject: (i) via a route selected from the group consisting of buccally, inhalation, intraarterially, intraarticularly, intracardially, intracerebroventricularly, intradermally, intramedullary, intramuscularly, intraperitoneally, intrathecally, intravenously, intraventricularly, nasally, ophthalmically, orally, rectally, sublabially, sublingually, subcutaneously, topically, and transdermally; and/or (ii) with a frequency selected from the group consisting of four times daily, three times daily, twice daily, once daily, once every two days, once every three days, once every four days, once every five days, once every six days, once a week, once every eight days, once every nine days, once every ten days, once every two weeks, once every three weeks, and once a month; and/or (iii) at a dosage (based on subject weight) selected from the group consisting of 0.1 μg/kg, 0.5 μg/kg, 1 μg/kg, 1.5 μg/kg, 2.0 μg/kg, 2.5 μg/kg, 3.0 μg/kg, 3.5 μg/kg, 4.0 μg/kg, 4.5 μg/kg, 5.0 μg/kg, 10 μg/kg, 15 μg/kg, 20 μg/kg, 25 μg/kg, 30 μg/kg, 35 μg/kg, 40 μg/kg, 45 μg/kg, 50 μg/kg, 55 μg/kg, 60 μg/kg, 65 μg/kg, 70 μg/kg, 75 μg/kg, 80 μg/kg, 85 μg/kg, 90 μg/kg, 95 μg/kg, 100 μg/kg, 150 μg/kg, 200 μg/kg, 250 μg/kg, 300 μg/kg, 350 μg/kg, 400 μg/kg, 450 μg/kg, 500 μg/kg, 550 μg/kg, 600 μg/kg, 650 μg/kg, 700 μg/kg, 750 μg/kg, 800 μg/kg, 850 μg/kg, 900 μg/kg, 1 mg/kg, 1.5 mg/kg, 2.0 mg/kg, 2.5 mg/kg, 3 mg/kg, 3.5 mg/kg, 4.0 mg/kg, 4.5 mg/kg, 5 mg/kg, 5.5 mg/kg, 6 mg/kg, 6.5 mg/kg, 7 mg/kg, 7.5 mg/kg, 8 mg/kg, 8.5 mg/kg, 9 mg/kg, 9.5 mg/kg, 10 mg/kg 10.5 mg/kg, 11 mg/kg, 11.5 mg/kg, 12 mg/kg, 12.5 mg/kg, 13 mg/kg, 13.5 mg/kg, 14 mg/kg, 14.5 mg/kg, 15 mg/kg, 16 mg/kg, 17 mg/kg, 18 mg/kg, 19 mg/kg, 20 mg/kg, 21 mg/kg, 22 mg/kg, 23 mg/kg, 24 mg/kg, 25 mg/kg, 30 mg/kg, 35 mg/kg, 40 mg/kg, 45 mg/kg, 50 mg/kg, 55 mg/kg, 60 mg/kg, 65 mg/kg, 70 mg/kg, 75 mg/kg, 80 mg/kg, 85 mg/kg, 90 mg/kg, 95 mg/kg, 100 mg/kg, 105 mg/kg, 110 mg/kg, 115 mg/kg, 120 mg/kg, 125 mg/kg, 130 mg/kg, 135 mg/kg, 140 mg/kg, 145 mg/kg, 150 mg/kg, 155 mg/kg, 160 mg/kg, 165 mg/kg, 170 mg/kg, 175 mg/kg, 180 mg/kg, 185 mg/kg, 190 mg/kg, 195 mg/kg, 200 mg/kg, and any fraction or integer in between any two of the preceding amounts.

In some embodiments, a NOS inhibitor composition comprising ARL-17477 and/or a salt thereof, such as, for example, ARL-17477 dihydrochloride, may be administered as a suitable dosage form, as described herein, to treat, ameliorate, and/or reduce the severity of pancreatic cancer or one or more symptoms thereof in a pancreatic cancer subject. In certain embodiments, an nNOS inhibitor composition comprising ARL-17477 and/or a salt thereof, such as, for example, ARL-17477 dihydrochloride, may be administered to a pancreatic cancer subject to treat, ameliorate, and/or reduce the severity of pancreatic cancer or one or more symptoms thereof in the pancreatic cancer subject: (i) via a route selected from the group consisting of buccally, inhalation, intraarterially, intraarticularly, intracardially, intracerebroventricularly, intradermally, intramedullary, intramuscularly, intraperitoneally, intrathecally, intravenously, intraventricularly, nasally, ophthalmically, orally, rectally, sublabially, sublingually, subcutaneously, topically, and transdermally; and/or (ii) with a frequency selected from the group consisting of four times daily, three times daily, twice daily, once daily, once every two days, once every three days, once every four days, once every five days, once every six days, once a week, once every eight days, once every nine days, once every ten days, once every two weeks, once every three weeks, and once a month; and/or (iii) at a dosage (based on subject weight) selected from the group consisting of 0.1 μg/kg, 0.5 μg/kg, 1 μg/kg, 1.5 μg/kg, 2.0 μg/kg, 2.5 μg/kg, 3.0 μg/kg, 3.5 μg/kg, 4.0 μg/kg, 4.5 μg/kg, 5.0 μg/kg, 10 μg/kg, 15 μg/kg, 20 μg/kg, 25 μg/kg, 30 μg/kg, 35 μg/kg, 40 μg/kg, 45 μg/kg, 50 μg/kg, 55 μg/kg, 60 μg/kg, 65 μg/kg, 70 μg/kg, 75 μg/kg, 80 μg/kg, 85 μg/kg, 90 μg/kg, 95 μg/kg, 100 μg/kg, 150 μg/kg, 200 μg/kg, 250 μg/kg, 300 μg/kg, 350 μg/kg, 400 μg/kg, 450 μg/kg, 500 μg/kg, 550 μg/kg, 600 μg/kg, 650 μg/kg, 700 μg/kg, 750 μg/kg, 800 μg/kg, 850 μg/kg, 900 μg/kg, 1 mg/kg, 1.5 mg/kg, 2.0 mg/kg, 2.5 mg/kg, 3 mg/kg, 3.5 mg/kg, 4.0 mg/kg, 4.5 mg/kg, 5 mg/kg, 5.5 mg/kg, 6 mg/kg, 6.5 mg/kg, 7 mg/kg, 7.5 mg/kg, 8 mg/kg, 8.5 mg/kg, 9 mg/kg, 9.5 mg/kg, 10 mg/kg 10.5 mg/kg, 11 mg/kg, 11.5 mg/kg, 12 mg/kg, 12.5 mg/kg, 13 mg/kg, 13.5 mg/kg, 14 mg/kg, 14.5 mg/kg, 15 mg/kg, 16 mg/kg, 17 mg/kg, 18 mg/kg, 19 mg/kg, 20 mg/kg, 21 mg/kg, 22 mg/kg, 23 mg/kg, 24 mg/kg, 25 mg/kg, 30 mg/kg, 35 mg/kg, 40 mg/kg, 45 mg/kg, 50 mg/kg, 55 mg/kg, 60 mg/kg, 65 mg/kg, 70 mg/kg, 75 mg/kg, 80 mg/kg, 85 mg/kg, 90 mg/kg, 95 mg/kg, 100 mg/kg, 105 mg/kg, 110 mg/kg, 115 mg/kg, 120 mg/kg, 125 mg/kg, 130 mg/kg, 135 mg/kg, 140 mg/kg, 145 mg/kg, 150 mg/kg, 155 mg/kg, 160 mg/kg, 165 mg/kg, 170 mg/kg, 175 mg/kg, 180 mg/kg, 185 mg/kg, 190 mg/kg, 195 mg/kg, 200 mg/kg, and any fraction or integer in between any two of the preceding amounts.

In certain embodiments, a NOS inhibitor composition comprising L-NNA and/or a salt thereof may be administered as a suitable dosage form, as described herein, to treat, ameliorate, and/or reduce the severity of pancreatic cancer or one or more symptoms thereof in a pancreatic cancer subject. In some embodiments, an nNOS inhibitor composition comprising L-NNA and/or a salt thereof may be administered to a pancreatic cancer subject to treat, ameliorate, and/or reduce the severity of pancreatic cancer or one or more symptoms thereof in the pancreatic cancer subject: (i) via a route selected from the group consisting of buccally, inhalation, intraarterially, intraarticularly, intracardially, intracerebroventricularly, intradermally, intramedullary, intramuscularly, intraperitoneally, intrathecally, intravenously, intraventricularly, nasally, ophthalmically, orally, rectally, sublabially, sublingually, subcutaneously, topically, and transdermally; and/or (ii) with a frequency selected from the group consisting of four times daily, three times daily, twice daily, once daily, once every two days, once every three days, once every four days, once every five days, once every six days, once a week, once every eight days, once every nine days, once every ten days, once every two weeks, once every three weeks, and once a month; and/or (iii) at a dosage (based on subject weight) selected from the group consisting of 0.1 μg/kg, 0.5 μg/kg, 1 μg/kg, 1.5 μg/kg, 2.0 μg/kg, 2.5 μg/kg, 3.0 μg/kg, 3.5 μg/kg, 4.0 μg/kg, 4.5 μg/kg, 5.0 μg/kg, 10 μg/kg, 15 μg/kg, 20 μg/kg, 25 μg/kg, 30 μg/kg, 35 μg/kg, 40 μg/kg, 45 μg/kg, 50 μg/kg, 55 μg/kg, 60 μg/kg, 65 μg/kg, 70 μg/kg, 75 μg/kg, 80 μg/kg, 85 μg/kg, 90 μg/kg, 95 μg/kg, 100 μg/kg, 150 μg/kg, 200 μg/kg, 250 μg/kg, 300 μg/kg, 350 μg/kg, 400 μg/kg, 450 μg/kg, 500 μg/kg, 550 μg/kg, 600 μg/kg, 650 μg/kg, 700 μg/kg, 750 μg/kg, 800 μg/kg, 850 μg/kg, 900 μg/kg, 1 mg/kg, 1.5 mg/kg, 2.0 mg/kg, 2.5 mg/kg, 3 mg/kg, 3.5 mg/kg, 4.0 mg/kg, 4.5 mg/kg, 5 mg/kg, 5.5 mg/kg, 6 mg/kg, 6.5 mg/kg, 7 mg/kg, 7.5 mg/kg, 8 mg/kg, 8.5 mg/kg, 9 mg/kg, 9.5 mg/kg, 10 mg/kg 10.5 mg/kg, 11 mg/kg, 11.5 mg/kg, 12 mg/kg, 12.5 mg/kg, 13 mg/kg, 13.5 mg/kg, 14 mg/kg, 14.5 mg/kg, 15 mg/kg, 16 mg/kg, 17 mg/kg, 18 mg/kg, 19 mg/kg, 20 mg/kg, 21 mg/kg, 22 mg/kg, 23 mg/kg, 24 mg/kg, 25 mg/kg, 30 mg/kg, 35 mg/kg, 40 mg/kg, 45 mg/kg, 50 mg/kg, 55 mg/kg, 60 mg/kg, 65 mg/kg, 70 mg/kg, 75 mg/kg, 80 mg/kg, 85 mg/kg, 90 mg/kg, 95 mg/kg, 100 mg/kg, 105 mg/kg, 110 mg/kg, 115 mg/kg, 120 mg/kg, 125 mg/kg, 130 mg/kg, 135 mg/kg, 140 mg/kg, 145 mg/kg, 150 mg/kg, 155 mg/kg, 160 mg/kg, 165 mg/kg, 170 mg/kg, 175 mg/kg, 180 mg/kg, 185 mg/kg, 190 mg/kg, 195 mg/kg, 200 mg/kg, and any fraction or integer in between any two of the preceding amounts.

In some embodiments, a NOS inhibitor composition comprising L-NAME and/or a salt thereof, such as, for example, L-NAME hydrochloride, may be administered as a suitable dosage form, as described herein, to treat, ameliorate, and/or reduce the severity of pancreatic cancer or one or more symptoms thereof in a pancreatic cancer subject. In certain embodiments, an nNOS inhibitor composition comprising L-NAME and/or a salt thereof, such as, for example, L-NAME hydrochloride, may be administered to a pancreatic cancer subject to treat, ameliorate, and/or reduce the severity of pancreatic cancer or one or more symptoms thereof in the pancreatic cancer subject: (i) via a route selected from the group consisting of buccally, inhalation, intraarterially, intraarticularly, intracardially, intracerebroventricularly, intradermally, intramedullary, intramuscularly, intraperitoneally, intrathecally, intravenously, intraventricularly, nasally, ophthalmically, orally, rectally, sublabially, sublingually, subcutaneously, topically, and transdermally; and/or (ii) with a frequency selected from the group consisting of four times daily, three times daily, twice daily, once daily, once every two days, once every three days, once every four days, once every five days, once every six days, once a week, once every eight days, once every nine days, once every ten days, once every two weeks, once every three weeks, and once a month; and/or (iii) at a dosage (based on subject weight) selected from the group consisting of 0.1 μg/kg, 0.5 μg/kg, 1 μg/kg, 1.5 μg/kg, 2.0 μg/kg, 2.5 μg/kg, 3.0 μg/kg, 3.5 μg/kg, 4.0 μg/kg, 4.5 μg/kg, 5.0 μg/kg, 10 μg/kg, 15 μg/kg, 20 μg/kg, 25 μg/kg, 30 μg/kg, 35 μg/kg, 40 μg/kg, 45 μg/kg, 50 μg/kg, 55 μg/kg, 60 μg/kg, 65 μg/kg, 70 μg/kg, 75 μg/kg, 80 μg/kg, 85 μg/kg, 90 μg/kg, 95 μg/kg, 100 μg/kg, 150 μg/kg, 200 μg/kg, 250 μg/kg, 300 μg/kg, 350 μg/kg, 400 μg/kg, 450 μg/kg, 500 μg/kg, 550 μg/kg, 600 μg/kg, 650 μg/kg, 700 μg/kg, 750 μg/kg, 800 μg/kg, 850 μg/kg, 900 μg/kg, 1 mg/kg, 1.5 mg/kg, 2.0 mg/kg, 2.5 mg/kg, 3 mg/kg, 3.5 mg/kg, 4.0 mg/kg, 4.5 mg/kg, 5 mg/kg, 5.5 mg/kg, 6 mg/kg, 6.5 mg/kg, 7 mg/kg, 7.5 mg/kg, 8 mg/kg, 8.5 mg/kg, 9 mg/kg, 9.5 mg/kg, 10 mg/kg 10.5 mg/kg, 11 mg/kg, 11.5 mg/kg, 12 mg/kg, 12.5 mg/kg, 13 mg/kg, 13.5 mg/kg, 14 mg/kg, 14.5 mg/kg, 15 mg/kg, 16 mg/kg, 17 mg/kg, 18 mg/kg, 19 mg/kg, 20 mg/kg, 21 mg/kg, 22 mg/kg, 23 mg/kg, 24 mg/kg, 25 mg/kg, 30 mg/kg, 35 mg/kg, 40 mg/kg, 45 mg/kg, 50 mg/kg, 55 mg/kg, 60 mg/kg, 65 mg/kg, 70 mg/kg, 75 mg/kg, 80 mg/kg, 85 mg/kg, 90 mg/kg, 95 mg/kg, 100 mg/kg, 105 mg/kg, 110 mg/kg, 115 mg/kg, 120 mg/kg, 125 mg/kg, 130 mg/kg, 135 mg/kg, 140 mg/kg, 145 mg/kg, 150 mg/kg, 155 mg/kg, 160 mg/kg, 165 mg/kg, 170 mg/kg, 175 mg/kg, 180 mg/kg, 185 mg/kg, 190 mg/kg, 195 mg/kg, 200 mg/kg, and any fraction or integer in between any two of the preceding amounts.

In certain embodiments, a NOS inhibitor composition comprising L-NMMA and/or a salt thereof, such as, for example, L-NMMA acetate, may be administered as a suitable dosage form, as described herein, to treat, ameliorate, and/or reduce the severity of pancreatic cancer or one or more symptoms thereof in a pancreatic cancer subject. In some embodiments, an nNOS inhibitor composition comprising L-NMMA and/or a salt thereof, such as, for example, L-NMMA acetate, may be administered to a pancreatic cancer subject to treat, ameliorate, and/or reduce the severity of pancreatic cancer or one or more symptoms thereof in the pancreatic cancer subject: (i) via a route selected from the group consisting of buccally, inhalation, intraarterially, intraarticularly, intracardially, intracerebroventricularly, intradermally, intramedullary, intramuscularly, intraperitoneally, intrathecally, intravenously, intraventricularly, nasally, ophthalmically, orally, rectally, sublabially, sublingually, subcutaneously, topically, and transdermally; and/or (ii) with a frequency selected from the group consisting of four times daily, three times daily, twice daily, once daily, once every two days, once every three days, once every four days, once every five days, once every six days, once a week, once every eight days, once every nine days, once every ten days, once every two weeks, once every three weeks, and once a month; and/or (iii) at a dosage (based on subject weight) selected from the group consisting of 0.1 μg/kg, 0.5 μg/kg, 1 μg/kg, 1.5 μg/kg, 2.0 μg/kg, 2.5 μg/kg, 3.0 μg/kg, 3.5 μg/kg, 4.0 μg/kg, 4.5 μg/kg, 5.0 μg/kg, 10 μg/kg, 15 μg/kg, 20 μg/kg, 25 μg/kg, 30 μg/kg, 35 μg/kg, 40 μg/kg, 45 μg/kg, 50 μg/kg, 55 μg/kg, 60 μg/kg, 65 μg/kg, 70 μg/kg, 75 μg/kg, 80 μg/kg, 85 μg/kg, 90 μg/kg, 95 μg/kg, 100 μg/kg, 150 μg/kg, 200 μg/kg, 250 μg/kg, 300 μg/kg, 350 μg/kg, 400 μg/kg, 450 μg/kg, 500 μg/kg, 550 μg/kg, 600 μg/kg, 650 μg/kg, 700 μg/kg, 750 μg/kg, 800 μg/kg, 850 μg/kg, 900 μg/kg, 1 mg/kg, 1.5 mg/kg, 2.0 mg/kg, 2.5 mg/kg, 3 mg/kg, 3.5 mg/kg, 4.0 mg/kg, 4.5 mg/kg, 5 mg/kg, 5.5 mg/kg, 6 mg/kg, 6.5 mg/kg, 7 mg/kg, 7.5 mg/kg, 8 mg/kg, 8.5 mg/kg, 9 mg/kg, 9.5 mg/kg, 10 mg/kg 10.5 mg/kg, 11 mg/kg, 11.5 mg/kg, 12 mg/kg, 12.5 mg/kg, 13 mg/kg, 13.5 mg/kg, 14 mg/kg, 14.5 mg/kg, 15 mg/kg, 16 mg/kg, 17 mg/kg, 18 mg/kg, 19 mg/kg, 20 mg/kg, 21 mg/kg, 22 mg/kg, 23 mg/kg, 24 mg/kg, 25 mg/kg, 30 mg/kg, 35 mg/kg, 40 mg/kg, 45 mg/kg, 50 mg/kg, 55 mg/kg, 60 mg/kg, 65 mg/kg, 70 mg/kg, 75 mg/kg, 80 mg/kg, 85 mg/kg, 90 mg/kg, 95 mg/kg, 100 mg/kg, 105 mg/kg, 110 mg/kg, 115 mg/kg, 120 mg/kg, 125 mg/kg, 130 mg/kg, 135 mg/kg, 140 mg/kg, 145 mg/kg, 150 mg/kg, 155 mg/kg, 160 mg/kg, 165 mg/kg, 170 mg/kg, 175 mg/kg, 180 mg/kg, 185 mg/kg, 190 mg/kg, 195 mg/kg, 200 mg/kg, and any fraction or integer in between any two of the preceding amounts.

In some embodiments, a NOS inhibitor composition comprising GW-274150 and/or a salt thereof, such as, for example, GW-274150 phosphate, may be administered as a suitable dosage form, as described herein, to treat, ameliorate, and/or reduce the severity of pancreatic cancer or one or more symptoms thereof in a pancreatic cancer subject. In certain embodiments, an nNOS inhibitor composition comprising GW-274150 and/or a salt thereof, such as, for example, GW-274150 phosphate, may be administered to a pancreatic cancer subject to treat, ameliorate, and/or reduce the severity of pancreatic cancer or one or more symptoms thereof in the pancreatic cancer subject: (i) via a route selected from the group consisting of buccally, inhalation, intraarterially, intraarticularly, intracardially, intracerebroventricularly, intradermally, intramedullary, intramuscularly, intraperitoneally, intrathecally, intravenously, intraventricularly, nasally, ophthalmically, orally, rectally, sublabially, sublingually, subcutaneously, topically, and transdermally; and/or (ii) with a frequency selected from the group consisting of four times daily, three times daily, twice daily, once daily, once every two days, once every three days, once every four days, once every five days, once every six days, once a week, once every eight days, once every nine days, once every ten days, once every two weeks, once every three weeks, and once a month; and/or (iii) at a dosage (based on subject weight) selected from the group consisting of 0.1 μg/kg, 0.5 μg/kg, 1 μg/kg, 1.5 μg/kg, 2.0 μg/kg, 2.5 μg/kg, 3.0 μg/kg, 3.5 μg/kg, 4.0 μg/kg, 4.5 μg/kg, 5.0 μg/kg, 10 μg/kg, 15 μg/kg, 20 μg/kg, 25 μg/kg, 30 μg/kg, 35 μg/kg, 40 μg/kg, 45 μg/kg, 50 μg/kg, 55 μg/kg, 60 μg/kg, 65 μg/kg, 70 μg/kg, 75 μg/kg, 80 μg/kg, 85 μg/kg, 90 μg/kg, 95 μg/kg, 100 μg/kg, 150 μg/kg, 200 μg/kg, 250 μg/kg, 300 μg/kg, 350 μg/kg, 400 μg/kg, 450 μg/kg, 500 μg/kg, 550 μg/kg, 600 μg/kg, 650 μg/kg, 700 μg/kg, 750 μg/kg, 800 μg/kg, 850 μg/kg, 900 μg/kg, 1 mg/kg, 1.5 mg/kg, 2.0 mg/kg, 2.5 mg/kg, 3 mg/kg, 3.5 mg/kg, 4.0 mg/kg, 4.5 mg/kg, 5 mg/kg, 5.5 mg/kg, 6 mg/kg, 6.5 mg/kg, 7 mg/kg, 7.5 mg/kg, 8 mg/kg, 8.5 mg/kg, 9 mg/kg, 9.5 mg/kg, 10 mg/kg 10.5 mg/kg, 11 mg/kg, 11.5 mg/kg, 12 mg/kg, 12.5 mg/kg, 13 mg/kg, 13.5 mg/kg, 14 mg/kg, 14.5 mg/kg, 15 mg/kg, 16 mg/kg, 17 mg/kg, 18 mg/kg, 19 mg/kg, 20 mg/kg, 21 mg/kg, 22 mg/kg, 23 mg/kg, 24 mg/kg, 25 mg/kg, 30 mg/kg, 35 mg/kg, 40 mg/kg, 45 mg/kg, 50 mg/kg, 55 mg/kg, 60 mg/kg, 65 mg/kg, 70 mg/kg, 75 mg/kg, 80 mg/kg, 85 mg/kg, 90 mg/kg, 95 mg/kg, 100 mg/kg, 105 mg/kg, 110 mg/kg, 115 mg/kg, 120 mg/kg, 125 mg/kg, 130 mg/kg, 135 mg/kg, 140 mg/kg, 145 mg/kg, 150 mg/kg, 155 mg/kg, 160 mg/kg, 165 mg/kg, 170 mg/kg, 175 mg/kg, 180 mg/kg, 185 mg/kg, 190 mg/kg, 195 mg/kg, 200 mg/kg, and any fraction or integer in between any two of the preceding amounts.

In certain embodiments, a NOS inhibitor composition comprising L-NIL and/or a salt thereof, such as, for example, L-NIL hydrochloride, may be administered as a suitable dosage form, as described herein, to treat, ameliorate, and/or reduce the severity of pancreatic cancer or one or more symptoms thereof in a pancreatic cancer subject. In some embodiments, an nNOS inhibitor composition comprising L-NIL and/or a salt thereof, such as, for example, L-NIL hydrochloride, may be administered to a pancreatic cancer subject to treat, ameliorate, and/or reduce the severity of pancreatic cancer or one or more symptoms thereof in the pancreatic cancer subject: (i) via a route selected from the group consisting of buccally, inhalation, intraarterially, intraarticularly, intracardially, intracerebroventricularly, intradermally, intramedullary, intramuscularly, intraperitoneally, intrathecally, intravenously, intraventricularly, nasally, ophthalmically, orally, rectally, sublabially, sublingually, subcutaneously, topically, and transdermally; and/or (ii) with a frequency selected from the group consisting of four times daily, three times daily, twice daily, once daily, once every two days, once every three days, once every four days, once every five days, once every six days, once a week, once every eight days, once every nine days, once every ten days, once every two weeks, once every three weeks, and once a month; and/or (iii) at a dosage (based on subject weight) selected from the group consisting of 0.1 μg/kg, 0.5 μg/kg, 1 μg/kg, 1.5 μg/kg, 2.0 μg/kg, 2.5 μg/kg, 3.0 μg/kg, 3.5 μg/kg, 4.0 μg/kg, 4.5 μg/kg, 5.0 μg/kg, 10 μg/kg, 15 μg/kg, 20 μg/kg, 25 μg/kg, 30 μg/kg, 35 μg/kg, 40 μg/kg, 45 μg/kg, 50 μg/kg, 55 μg/kg, 60 μg/kg, 65 μg/kg, 70 μg/kg, 75 μg/kg, 80 μg/kg, 85 μg/kg, 90 μg/kg, 95 μg/kg, 100 μg/kg, 150 μg/kg, 200 μg/kg, 250 μg/kg, 300 μg/kg, 350 μg/kg, 400 μg/kg, 450 μg/kg, 500 μg/kg, 550 μg/kg, 600 μg/kg, 650 μg/kg, 700 μg/kg, 750 μg/kg, 800 μg/kg, 850 μg/kg, 900 μg/kg, 1 mg/kg, 1.5 mg/kg, 2.0 mg/kg, 2.5 mg/kg, 3 mg/kg, 3.5 mg/kg, 4.0 mg/kg, 4.5 mg/kg, 5 mg/kg, 5.5 mg/kg, 6 mg/kg, 6.5 mg/kg, 7 mg/kg, 7.5 mg/kg, 8 mg/kg, 8.5 mg/kg, 9 mg/kg, 9.5 mg/kg, 10 mg/kg 10.5 mg/kg, 11 mg/kg, 11.5 mg/kg, 12 mg/kg, 12.5 mg/kg, 13 mg/kg, 13.5 mg/kg, 14 mg/kg, 14.5 mg/kg, 15 mg/kg, 16 mg/kg, 17 mg/kg, 18 mg/kg, 19 mg/kg, 20 mg/kg, 21 mg/kg, 22 mg/kg, 23 mg/kg, 24 mg/kg, 25 mg/kg, 30 mg/kg, 35 mg/kg, 40 mg/kg, 45 mg/kg, 50 mg/kg, 55 mg/kg, 60 mg/kg, 65 mg/kg, 70 mg/kg, 75 mg/kg, 80 mg/kg, 85 mg/kg, 90 mg/kg, 95 mg/kg, 100 mg/kg, 105 mg/kg, 110 mg/kg, 115 mg/kg, 120 mg/kg, 125 mg/kg, 130 mg/kg, 135 mg/kg, 140 mg/kg, 145 mg/kg, 150 mg/kg, 155 mg/kg, 160 mg/kg, 165 mg/kg, 170 mg/kg, 175 mg/kg, 180 mg/kg, 185 mg/kg, 190 mg/kg, 195 mg/kg, 200 mg/kg, and any fraction or integer in between any two of the preceding amounts.

In some embodiments, a NOS inhibitor composition comprising 1400W and/or a salt thereof, such as, for example, 1400W dihydrochloride, may be administered as a suitable dosage form, as described herein, to treat, ameliorate, and/or reduce the severity of pancreatic cancer or one or more symptoms thereof in a pancreatic cancer subject. In certain embodiments, an nNOS inhibitor composition comprising 1400W and/or a salt thereof, such as, for example, 1400W dihydrochloride, may be administered to a pancreatic cancer subject to treat, ameliorate, and/or reduce the severity of pancreatic cancer or one or more symptoms thereof in the pancreatic cancer subject: (i) via a route selected from the group consisting of buccally, inhalation, intraarterially, intraarticularly, intracardially, intracerebroventricularly, intradermally, intramedullary, intramuscularly, intraperitoneally, intrathecally, intravenously, intraventricularly, nasally, ophthalmically, orally, rectally, sublabially, sublingually, subcutaneously, topically, and transdermally; and/or (ii) with a frequency selected from the group consisting of four times daily, three times daily, twice daily, once daily, once every two days, once every three days, once every four days, once every five days, once every six days, once a week, once every eight days, once every nine days, once every ten days, once every two weeks, once every three weeks, and once a month; and/or (iii) at a dosage (based on subject weight) selected from the group consisting of 0.1 μg/kg, 0.5 μg/kg, 1 μg/kg, 1.5 μg/kg, 2.0 μg/kg, 2.5 μg/kg, 3.0 μg/kg, 3.5 μg/kg, 4.0 μg/kg, 4.5 μg/kg, 5.0 μg/kg, 10 μg/kg, 15 μg/kg, 20 μg/kg, 25 μg/kg, 30 μg/kg, 35 μg/kg, 40 μg/kg, 45 μg/kg, 50 μg/kg, 55 μg/kg, 60 μg/kg, 65 μg/kg, 70 μg/kg, 75 μg/kg, 80 μg/kg, 85 μg/kg, 90 μg/kg, 95 μg/kg, 100 μg/kg, 150 μg/kg, 200 μg/kg, 250 μg/kg, 300 μg/kg, 350 μg/kg, 400 μg/kg, 450 μg/kg, 500 μg/kg, 550 μg/kg, 600 μg/kg, 650 μg/kg, 700 μg/kg, 750 μg/kg, 800 μg/kg, 850 μg/kg, 900 μg/kg, 1 mg/kg, 1.5 mg/kg, 2.0 mg/kg, 2.5 mg/kg, 3 mg/kg, 3.5 mg/kg, 4.0 mg/kg, 4.5 mg/kg, 5 mg/kg, 5.5 mg/kg, 6 mg/kg, 6.5 mg/kg, 7 mg/kg, 7.5 mg/kg, 8 mg/kg, 8.5 mg/kg, 9 mg/kg, 9.5 mg/kg, 10 mg/kg 10.5 mg/kg, 11 mg/kg, 11.5 mg/kg, 12 mg/kg, 12.5 mg/kg, 13 mg/kg, 13.5 mg/kg, 14 mg/kg, 14.5 mg/kg, 15 mg/kg, 16 mg/kg, 17 mg/kg, 18 mg/kg, 19 mg/kg, 20 mg/kg, 21 mg/kg, 22 mg/kg, 23 mg/kg, 24 mg/kg, 25 mg/kg, 30 mg/kg, 35 mg/kg, 40 mg/kg, 45 mg/kg, 50 mg/kg, 55 mg/kg, 60 mg/kg, 65 mg/kg, 70 mg/kg, 75 mg/kg, 80 mg/kg, 85 mg/kg, 90 mg/kg, 95 mg/kg, 100 mg/kg, 105 mg/kg, 110 mg/kg, 115 mg/kg, 120 mg/kg, 125 mg/kg, 130 mg/kg, 135 mg/kg, 140 mg/kg, 145 mg/kg, 150 mg/kg, 155 mg/kg, 160 mg/kg, 165 mg/kg, 170 mg/kg, 175 mg/kg, 180 mg/kg, 185 mg/kg, 190 mg/kg, 195 mg/kg, 200 mg/kg, and any fraction or integer in between any two of the preceding amounts.

In certain embodiments, a NOS inhibitor composition comprising OKN-007 and/or a salt thereof, such as, for example, OKN-007 sodium, may be administered as a suitable dosage form, as described herein, to treat, ameliorate, and/or reduce the severity of pancreatic cancer or one or more symptoms thereof in a pancreatic cancer subject. In some embodiments, an nNOS inhibitor composition comprising OKN-007 and/or a salt thereof, such as, for example, OKN-007 sodium, may be administered to a pancreatic cancer subject to treat, ameliorate, and/or reduce the severity of pancreatic cancer or one or more symptoms thereof in the pancreatic cancer subject: (i) via a route selected from the group consisting of buccally, inhalation, intraarterially, intraarticularly, intracardially, intracerebroventricularly, intradermally, intramedullary, intramuscularly, intraperitoneally, intrathecally, intravenously, intraventricularly, nasally, ophthalmically, orally, rectally, sublabially, sublingually, subcutaneously, topically, and transdermally; and/or (ii) with a frequency selected from the group consisting of four times daily, three times daily, twice daily, once daily, once every two days, once every three days, once every four days, once every five days, once every six days, once a week, once every eight days, once every nine days, once every ten days, once every two weeks, once every three weeks, and once a month; and/or (iii) at a dosage (based on subject weight) selected from the group consisting of 0.1 μg/kg, 0.5 μg/kg, 1 μg/kg, 1.5 μg/kg, 2.0 μg/kg, 2.5 μg/kg, 3.0 μg/kg, 3.5 μg/kg, 4.0 μg/kg, 4.5 μg/kg, 5.0 μg/kg, 10 μg/kg, 15 μg/kg, 20 μg/kg, 25 μg/kg, 30 μg/kg, 35 μg/kg, 40 μg/kg, 45 μg/kg, 50 μg/kg, 55 μg/kg, 60 μg/kg, 65 μg/kg, 70 μg/kg, 75 μg/kg, 80 μg/kg, 85 μg/kg, 90 μg/kg, 95 μg/kg, 100 μg/kg, 150 μg/kg, 200 μg/kg, 250 μg/kg, 300 μg/kg, 350 μg/kg, 400 μg/kg, 450 μg/kg, 500 μg/kg, 550 μg/kg, 600 μg/kg, 650 μg/kg, 700 μg/kg, 750 μg/kg, 800 μg/kg, 850 μg/kg, 900 μg/kg, 1 mg/kg, 1.5 mg/kg, 2.0 mg/kg, 2.5 mg/kg, 3 mg/kg, 3.5 mg/kg, 4.0 mg/kg, 4.5 mg/kg, 5 mg/kg, 5.5 mg/kg, 6 mg/kg, 6.5 mg/kg, 7 mg/kg, 7.5 mg/kg, 8 mg/kg, 8.5 mg/kg, 9 mg/kg, 9.5 mg/kg, 10 mg/kg 10.5 mg/kg, 11 mg/kg, 11.5 mg/kg, 12 mg/kg, 12.5 mg/kg, 13 mg/kg, 13.5 mg/kg, 14 mg/kg, 14.5 mg/kg, 15 mg/kg, 16 mg/kg, 17 mg/kg, 18 mg/kg, 19 mg/kg, 20 mg/kg, 21 mg/kg, 22 mg/kg, 23 mg/kg, 24 mg/kg, 25 mg/kg, 30 mg/kg, 35 mg/kg, 40 mg/kg, 45 mg/kg, 50 mg/kg, 55 mg/kg, 60 mg/kg, 65 mg/kg, 70 mg/kg, 75 mg/kg, 80 mg/kg, 85 mg/kg, 90 mg/kg, 95 mg/kg, 100 mg/kg, 105 mg/kg, 110 mg/kg, 115 mg/kg, 120 mg/kg, 125 mg/kg, 130 mg/kg, 135 mg/kg, 140 mg/kg, 145 mg/kg, 150 mg/kg, 155 mg/kg, 160 mg/kg, 165 mg/kg, 170 mg/kg, 175 mg/kg, 180 mg/kg, 185 mg/kg, 190 mg/kg, 195 mg/kg, 200 mg/kg, and any fraction or integer in between any two of the preceding amounts.

In some embodiments, a NOS inhibitor composition comprising BYK-191023 and/or a salt thereof, such as, for example, BYK-191023 dihydrochloride, may be administered as a suitable dosage form, as described herein, to treat, ameliorate, and/or reduce the severity of pancreatic cancer or one or more symptoms thereof in a pancreatic cancer subject. In certain embodiments, an nNOS inhibitor composition comprising BYK-191023 and/or a salt thereof, such as, for example, BYK-191023 dihydrochloride, may be administered to a pancreatic cancer subject to treat, ameliorate, and/or reduce the severity of pancreatic cancer or one or more symptoms thereof in the pancreatic cancer subject: (i) via a route selected from the group consisting of buccally, inhalation, intraarterially, intraarticularly, intracardially, intracerebroventricularly, intradermally, intramedullary, intramuscularly, intraperitoneally, intrathecally, intravenously, intraventricularly, nasally, ophthalmically, orally, rectally, sublabially, sublingually, subcutaneously, topically, and transdermally; and/or (ii) with a frequency selected from the group consisting of four times daily, three times daily, twice daily, once daily, once every two days, once every three days, once every four days, once every five days, once every six days, once a week, once every eight days, once every nine days, once every ten days, once every two weeks, once every three weeks, and once a month; and/or (iii) at a dosage (based on subject weight) selected from the group consisting of 0.1 μg/kg, 0.5 μg/kg, 1 μg/kg, 1.5 μg/kg, 2.0 μg/kg, 2.5 μg/kg, 3.0 μg/kg, 3.5 μg/kg, 4.0 μg/kg, 4.5 μg/kg, 5.0 μg/kg, 10 μg/kg, 15 μg/kg, 20 μg/kg, 25 μg/kg, 30 μg/kg, 35 μg/kg, 40 μg/kg, 45 μg/kg, 50 μg/kg, 55 μg/kg, 60 μg/kg, 65 μg/kg, 70 μg/kg, 75 μg/kg, 80 μg/kg, 85 μg/kg, 90 μg/kg, 95 μg/kg, 100 μg/kg, 150 μg/kg, 200 μg/kg, 250 μg/kg, 300 μg/kg, 350 μg/kg, 400 μg/kg, 450 μg/kg, 500 μg/kg, 550 μg/kg, 600 μg/kg, 650 μg/kg, 700 μg/kg, 750 μg/kg, 800 μg/kg, 850 μg/kg, 900 μg/kg, 1 mg/kg, 1.5 mg/kg, 2.0 mg/kg, 2.5 mg/kg, 3 mg/kg, 3.5 mg/kg, 4.0 mg/kg, 4.5 mg/kg, 5 mg/kg, 5.5 mg/kg, 6 mg/kg, 6.5 mg/kg, 7 mg/kg, 7.5 mg/kg, 8 mg/kg, 8.5 mg/kg, 9 mg/kg, 9.5 mg/kg, 10 mg/kg 10.5 mg/kg, 11 mg/kg, 11.5 mg/kg, 12 mg/kg, 12.5 mg/kg, 13 mg/kg, 13.5 mg/kg, 14 mg/kg, 14.5 mg/kg, 15 mg/kg, 16 mg/kg, 17 mg/kg, 18 mg/kg, 19 mg/kg, 20 mg/kg, 21 mg/kg, 22 mg/kg, 23 mg/kg, 24 mg/kg, 25 mg/kg, 30 mg/kg, 35 mg/kg, 40 mg/kg, 45 mg/kg, 50 mg/kg, 55 mg/kg, 60 mg/kg, 65 mg/kg, 70 mg/kg, 75 mg/kg, 80 mg/kg, 85 mg/kg, 90 mg/kg, 95 mg/kg, 100 mg/kg, 105 mg/kg, 110 mg/kg, 115 mg/kg, 120 mg/kg, 125 mg/kg, 130 mg/kg, 135 mg/kg, 140 mg/kg, 145 mg/kg, 150 mg/kg, 155 mg/kg, 160 mg/kg, 165 mg/kg, 170 mg/kg, 175 mg/kg, 180 mg/kg, 185 mg/kg, 190 mg/kg, 195 mg/kg, 200 mg/kg, and any fraction or integer in between any two of the preceding amounts.

In certain embodiments, a NOS inhibitor composition comprising CM544 and/or a salt thereof, such as, for example, CM544 dihydrochloride, may be administered as a suitable dosage form, as described herein, to treat, ameliorate, and/or reduce the severity of pancreatic cancer or one or more symptoms thereof in a pancreatic cancer subject. In some embodiments, an nNOS inhibitor composition comprising CM544 and/or a salt thereof, such as, for example, CM544 dihydrochloride, may be administered to a pancreatic cancer subject to treat, ameliorate, and/or reduce the severity of pancreatic cancer or one or more symptoms thereof in the pancreatic cancer subject: (i) via a route selected from the group consisting of buccally, inhalation, intraarterially, intraarticularly, intracardially, intracerebroventricularly, intradermally, intramedullary, intramuscularly, intraperitoneally, intrathecally, intravenously, intraventricularly, nasally, ophthalmically, orally, rectally, sublabially, sublingually, subcutaneously, topically, and transdermally; and/or (ii) with a frequency selected from the group consisting of four times daily, three times daily, twice daily, once daily, once every two days, once every three days, once every four days, once every five days, once every six days, once a week, once every eight days, once every nine days, once every ten days, once every two weeks, once every three weeks, and once a month; and/or (iii) at a dosage (based on subject weight) selected from the group consisting of 0.1 μg/kg, 0.5 μg/kg, 1 μg/kg, 1.5 μg/kg, 2.0 μg/kg, 2.5 μg/kg, 3.0 μg/kg, 3.5 μg/kg, 4.0 μg/kg, 4.5 μg/kg, 5.0 μg/kg, 10 μg/kg, 15 μg/kg, 20 μg/kg, 25 μg/kg, 30 μg/kg, 35 μg/kg, 40 μg/kg, 45 μg/kg, 50 μg/kg, 55 μg/kg, 60 μg/kg, 65 μg/kg, 70 μg/kg, 75 μg/kg, 80 μg/kg, 85 μg/kg, 90 μg/kg, 95 μg/kg, 100 μg/kg, 150 μg/kg, 200 μg/kg, 250 μg/kg, 300 μg/kg, 350 μg/kg, 400 μg/kg, 450 μg/kg, 500 μg/kg, 550 μg/kg, 600 μg/kg, 650 μg/kg, 700 μg/kg, 750 μg/kg, 800 μg/kg, 850 μg/kg, 900 μg/kg, 1 mg/kg, 1.5 mg/kg, 2.0 mg/kg, 2.5 mg/kg, 3 mg/kg, 3.5 mg/kg, 4.0 mg/kg, 4.5 mg/kg, 5 mg/kg, 5.5 mg/kg, 6 mg/kg, 6.5 mg/kg, 7 mg/kg, 7.5 mg/kg, 8 mg/kg, 8.5 mg/kg, 9 mg/kg, 9.5 mg/kg, 10 mg/kg 10.5 mg/kg, 11 mg/kg, 11.5 mg/kg, 12 mg/kg, 12.5 mg/kg, 13 mg/kg, 13.5 mg/kg, 14 mg/kg, 14.5 mg/kg, 15 mg/kg, 16 mg/kg, 17 mg/kg, 18 mg/kg, 19 mg/kg, 20 mg/kg, 21 mg/kg, 22 mg/kg, 23 mg/kg, 24 mg/kg, 25 mg/kg, 30 mg/kg, 35 mg/kg, 40 mg/kg, 45 mg/kg, 50 mg/kg, 55 mg/kg, 60 mg/kg, 65 mg/kg, 70 mg/kg, 75 mg/kg, 80 mg/kg, 85 mg/kg, 90 mg/kg, 95 mg/kg, 100 mg/kg, 105 mg/kg, 110 mg/kg, 115 mg/kg, 120 mg/kg, 125 mg/kg, 130 mg/kg, 135 mg/kg, 140 mg/kg, 145 mg/kg, 150 mg/kg, 155 mg/kg, 160 mg/kg, 165 mg/kg, 170 mg/kg, 175 mg/kg, 180 mg/kg, 185 mg/kg, 190 mg/kg, 195 mg/kg, 200 mg/kg, and any fraction or integer in between any two of the preceding amounts.

In certain embodiments, an nNOS inhibitor composition comprising one or more dsRNAs each comprising at least 30 base pairs, may be administered as a suitable dosage form, as described herein, to treat, ameliorate, and/or reduce the severity of pancreatic cancer or one or more symptoms thereof in a pancreatic cancer subject. In some embodiments, an nNOS inhibitor composition comprising one or more dsRNAs, each comprising at least 30 base pairs, may be administered to a pancreatic cancer subject to treat, ameliorate, and/or reduce the severity of pancreatic cancer or one or more symptoms thereof in the pancreatic cancer subject: (i) via a route selected from the group consisting of buccally, inhalation, intraarterially, intraarticularly, intracardially, intracerebroventricularly, intradermally, intramedullary, intramuscularly, intraperitoneally, intrathecally, intravenously, intraventricularly, nasally, ophthalmically, orally, rectally, sublabially, sublingually, subcutaneously, topically, and transdermally; and/or (ii) with a frequency selected from the group consisting of four times daily, three times daily, twice daily, once daily, once every two days, once every three days, once every four days, once every five days, once every six days, once a week, once every eight days, once every nine days, once every ten days, once every two weeks, once every three weeks, and once a month; and/or (iii) at a dosage (based on subject weight) selected from the group consisting of 0.1 μg/kg, 0.5 μg/kg, 1 μg/kg, 1.5 μg/kg, 2.0 μg/kg, 2.5 μg/kg, 3.0 μg/kg, 3.5 μg/kg, 4.0 μg/kg, 4.5 μg/kg, 5.0 μg/kg, 10 μg/kg, 15 μg/kg, 20 μg/kg, 25 μg/kg, 30 μg/kg, 35 μg/kg, 40 μg/kg, 45 μg/kg, 50 μg/kg, 55 μg/kg, 60 μg/kg, 65 μg/kg, 70 μg/kg, 75 μg/kg, 80 μg/kg, 85 μg/kg, 90 μg/kg, 95 μg/kg, 100 μg/kg, 150 μg/kg, 200 μg/kg, 250 μg/kg, 300 μg/kg, 350 μg/kg, 400 μg/kg, 450 μg/kg, 500 μg/kg, 550 μg/kg, 600 μg/kg, 650 μg/kg, 700 μg/kg, 750 μg/kg, 800 μg/kg, 850 μg/kg, 900 μg/kg, 1 mg/kg, 1.5 mg/kg, 2.0 mg/kg, 2.5 mg/kg, 3 mg/kg, 3.5 mg/kg, 4.0 mg/kg, 4.5 mg/kg, 5 mg/kg, 5.5 mg/kg, 6 mg/kg, 6.5 mg/kg, 7 mg/kg, 7.5 mg/kg, 8 mg/kg, 8.5 mg/kg, 9 mg/kg, 9.5 mg/kg, 10 mg/kg 10.5 mg/kg, 11 mg/kg, 11.5 mg/kg, 12 mg/kg, 12.5 mg/kg, 13 mg/kg, 13.5 mg/kg, 14 mg/kg, 14.5 mg/kg, 15 mg/kg, 16 mg/kg, 17 mg/kg, 18 mg/kg, 19 mg/kg, 20 mg/kg, 21 mg/kg, 22 mg/kg, 23 mg/kg, 24 mg/kg, 25 mg/kg, 30 mg/kg, 35 mg/kg, 40 mg/kg, 45 mg/kg, 50 mg/kg, 55 mg/kg, 60 mg/kg, 65 mg/kg, 70 mg/kg, 75 mg/kg, 80 mg/kg, 85 mg/kg, 90 mg/kg, 95 mg/kg, 100 mg/kg, 105 mg/kg, 110 mg/kg, 115 mg/kg, 120 mg/kg, 125 mg/kg, 130 mg/kg, 135 mg/kg, 140 mg/kg, 145 mg/kg, 150 mg/kg, 155 mg/kg, 160 mg/kg, 165 mg/kg, 170 mg/kg, 175 mg/kg, 180 mg/kg, 185 mg/kg, 190 mg/kg, 195 mg/kg, 200 mg/kg, and any fraction or integer in between any two of the preceding amounts.

In some embodiments, an nNOS inhibitor composition comprising one or more siRNAs, may be administered as a suitable dosage form, as described herein, to treat, ameliorate, and/or reduce the severity of pancreatic cancer or one or more symptoms thereof in a pancreatic cancer subject. In certain embodiments, an nNOS inhibitor composition comprising one or more siRNAs selected from the group consisting of SEQ ID NO. 4, SEQ ID NO. 5, SEQ ID NO. 6, SEQ ID NO. 7, SEQ ID NO. 8, and any combination thereof, may be administered as a suitable dosage form, as described herein, to treat, ameliorate, and/or reduce the severity of pancreatic cancer or one or more symptoms thereof in a pancreatic cancer subject. In some embodiments, an nNOS inhibitor composition comprising one or more siRNAs selected from the group consisting of SEQ ID NO. 4, SEQ ID NO. 5, SEQ ID NO. 6, SEQ ID NO. 7, SEQ ID NO. 8, and any combination thereof, may be administered to a pancreatic cancer subject to treat, ameliorate, and/or reduce the severity of pancreatic cancer or one or more symptoms thereof in the pancreatic cancer subject: (i) via a route selected from the group consisting of buccally, inhalation, intraarterially, intraarticularly, intracardially, intracerebroventricularly, intradermally, intramedullary, intramuscularly, intraperitoneally, intrathecally, intravenously, intraventricularly, nasally, ophthalmically, orally, rectally, sublabially, sublingually, subcutaneously, topically, and transdermally; and/or (ii) with a frequency selected from the group consisting of four times daily, three times daily, twice daily, once daily, once every two days, once every three days, once every four days, once every five days, once every six days, once a week, once every eight days, once every nine days, once every ten days, once every two weeks, once every three weeks, and once a month; and/or (iii) at a dosage (based on subject weight) selected from the group consisting of 0.1 μg/kg, 0.5 μg/kg, 1 μg/kg, 1.5 μg/kg, 2.0 μg/kg, 2.5 μg/kg, 3.0 μg/kg, 3.5 μg/kg, 4.0 μg/kg, 4.5 μg/kg, 5.0 μg/kg, 10 μg/kg, 15 μg/kg, 20 μg/kg, 25 μg/kg, 30 μg/kg, 35 μg/kg, 40 μg/kg, 45 μg/kg, 50 μg/kg, 55 μg/kg, 60 μg/kg, 65 μg/kg, 70 μg/kg, 75 μg/kg, 80 μg/kg, 85 μg/kg, 90 μg/kg, 95 μg/kg, 100 μg/kg, 150 μg/kg, 200 μg/kg, 250 μg/kg, 300 μg/kg, 350 μg/kg, 400 μg/kg, 450 μg/kg, 500 μg/kg, 550 μg/kg, 600 μg/kg, 650 μg/kg, 700 μg/kg, 750 μg/kg, 800 μg/kg, 850 μg/kg, 900 μg/kg, 1 mg/kg, 1.5 mg/kg, 2.0 mg/kg, 2.5 mg/kg, 3 mg/kg, 3.5 mg/kg, 4.0 mg/kg, 4.5 mg/kg, 5 mg/kg, 5.5 mg/kg, 6 mg/kg, 6.5 mg/kg, 7 mg/kg, 7.5 mg/kg, 8 mg/kg, 8.5 mg/kg, 9 mg/kg, 9.5 mg/kg, 10 mg/kg 10.5 mg/kg, 11 mg/kg, 11.5 mg/kg, 12 mg/kg, 12.5 mg/kg, 13 mg/kg, 13.5 mg/kg, 14 mg/kg, 14.5 mg/kg, 15 mg/kg, 16 mg/kg, 17 mg/kg, 18 mg/kg, 19 mg/kg, 20 mg/kg, 21 mg/kg, 22 mg/kg, 23 mg/kg, 24 mg/kg, 25 mg/kg, 30 mg/kg, 35 mg/kg, 40 mg/kg, 45 mg/kg, 50 mg/kg, 55 mg/kg, 60 mg/kg, 65 mg/kg, 70 mg/kg, 75 mg/kg, 80 mg/kg, 85 mg/kg, 90 mg/kg, 95 mg/kg, 100 mg/kg, 105 mg/kg, 110 mg/kg, 115 mg/kg, 120 mg/kg, 125 mg/kg, 130 mg/kg, 135 mg/kg, 140 mg/kg, 145 mg/kg, 150 mg/kg, 155 mg/kg, 160 mg/kg, 165 mg/kg, 170 mg/kg, 175 mg/kg, 180 mg/kg, 185 mg/kg, 190 mg/kg, 195 mg/kg, 200 mg/kg, and any fraction or integer in between any two of the preceding amounts.

In some embodiments, an nNOS inhibitor composition comprising one or more shRNAs, may be administered as a suitable dosage form, as described herein, to treat, ameliorate, and/or reduce the severity of pancreatic cancer or one or more symptoms thereof in a pancreatic cancer subject. In certain embodiments, an nNOS inhibitor composition comprising one or more shRNAs selected from the group consisting of SEQ ID NO. 9, SEQ ID NO. 10, SEQ ID NO. 11, SEQ ID NO. 12, SEQ ID NO. 13, SEQ ID NO. 14, and any combination thereof, may be administered as a suitable dosage form, as described herein, to treat, ameliorate, and/or reduce the severity of pancreatic cancer or one or more symptoms thereof in a pancreatic cancer subject. In some embodiments, an nNOS inhibitor composition comprising one or more shRNAs selected from the group consisting of SEQ ID NO. 9, SEQ ID NO. 10, SEQ ID NO. 11, SEQ ID NO. 12, SEQ ID NO. 13, SEQ ID NO. 14, and any combination thereof, may be administered to a pancreatic cancer subject to treat, ameliorate, and/or reduce the severity of pancreatic cancer or one or more symptoms thereof in the pancreatic cancer subject: (i) via a route selected from the group consisting of buccally, inhalation, intraarterially, intraarticularly, intracardially, intracerebroventricularly, intradermally, intramedullary, intramuscularly, intraperitoneally, intrathecally, intravenously, intraventricularly, nasally, ophthalmically, orally, rectally, sublabially, sublingually, subcutaneously, topically, and transdermally; and/or (ii) with a frequency selected from the group consisting of four times daily, three times daily, twice daily, once daily, once every two days, once every three days, once every four days, once every five days, once every six days, once a week, once every eight days, once every nine days, once every ten days, once every two weeks, once every three weeks, and once a month; and/or (iii) at a dosage (based on subject weight) selected from the group consisting of 0.1 μg/kg, 0.5 μg/kg, 1 μg/kg, 1.5 μg/kg, 2.0 μg/kg, 2.5 μg/kg, 3.0 μg/kg, 3.5 μg/kg, 4.0 μg/kg, 4.5 μg/kg, 5.0 μg/kg, 10 μg/kg, 15 μg/kg, 20 μg/kg, 25 μg/kg, 30 μg/kg, 35 μg/kg, 40 μg/kg, 45 μg/kg, 50 μg/kg, 55 μg/kg, 60 μg/kg, 65 μg/kg, 70 μg/kg, 75 μg/kg, 80 μg/kg, 85 μg/kg, 90 μg/kg, 95 μg/kg, 100 μg/kg, 150 μg/kg, 200 μg/kg, 250 μg/kg, 300 μg/kg, 350 μg/kg, 400 μg/kg, 450 μg/kg, 500 μg/kg, 550 μg/kg, 600 μg/kg, 650 μg/kg, 700 μg/kg, 750 μg/kg, 800 μg/kg, 850 μg/kg, 900 μg/kg, 1 mg/kg, 1.5 mg/kg, 2.0 mg/kg, 2.5 mg/kg, 3 mg/kg, 3.5 mg/kg, 4.0 mg/kg, 4.5 mg/kg, 5 mg/kg, 5.5 mg/kg, 6 mg/kg, 6.5 mg/kg, 7 mg/kg, 7.5 mg/kg, 8 mg/kg, 8.5 mg/kg, 9 mg/kg, 9.5 mg/kg, 10 mg/kg 10.5 mg/kg, 11 mg/kg, 11.5 mg/kg, 12 mg/kg, 12.5 mg/kg, 13 mg/kg, 13.5 mg/kg, 14 mg/kg, 14.5 mg/kg, 15 mg/kg, 16 mg/kg, 17 mg/kg, 18 mg/kg, 19 mg/kg, 20 mg/kg, 21 mg/kg, 22 mg/kg, 23 mg/kg, 24 mg/kg, 25 mg/kg, 30 mg/kg, 35 mg/kg, 40 mg/kg, 45 mg/kg, 50 mg/kg, 55 mg/kg, 60 mg/kg, 65 mg/kg, 70 mg/kg, 75 mg/kg, 80 mg/kg, 85 mg/kg, 90 mg/kg, 95 mg/kg, 100 mg/kg, 105 mg/kg, 110 mg/kg, 115 mg/kg, 120 mg/kg, 125 mg/kg, 130 mg/kg, 135 mg/kg, 140 mg/kg, 145 mg/kg, 150 mg/kg, 155 mg/kg, 160 mg/kg, 165 mg/kg, 170 mg/kg, 175 mg/kg, 180 mg/kg, 185 mg/kg, 190 mg/kg, 195 mg/kg, 200 mg/kg, and any fraction or integer in between any two of the preceding amounts.

In some embodiments, an nNOS inhibitor composition comprising one or more miRNAs, may be administered as a suitable dosage form, as described herein, to treat, ameliorate, and/or reduce the severity of pancreatic cancer or one or more symptoms thereof in a pancreatic cancer subject. In certain embodiments, an nNOS inhibitor composition comprising one or more miRNAs selected from the group consisting of miR-146a, hsa-miR-146a-5p, hsa-miR-1273f, hsa-miR-143-3p, hsa-miR-147a, hsa-miR-3622a-5p, hsa-miR-3911, hsa-miR-4269, hsa-miR-4708-5p, hsa-miR-4710, hsa-miR-4770, hsa-miR-4792, hsa-miR-574-5p, hsa-miR-6088, hsa-miR-644a, hsa-miR-6715b-5p, hsa-miR-6768-5p, hsa-miR-6867-5p, hsa-miR-7855-5p, hsa-miR-8485, and any combination thereof, may be administered as a suitable dosage form, as described herein, to treat, ameliorate, and/or reduce the severity of pancreatic cancer or one or more symptoms thereof in a pancreatic cancer subject. In some embodiments, an nNOS inhibitor composition comprising one or more miRNAs selected from the group consisting of miR-146a, hsa-miR-146a-5p, hsa-miR-1273f, hsa-miR-143-3p, hsa-miR-147a, hsa-miR-3622a-5p, hsa-miR-3911, hsa-miR-4269, hsa-miR-4708-5p, hsa-miR-4710, hsa-miR-4770, hsa-miR-4792, hsa-miR-574-5p, hsa-miR-6088, hsa-miR-644a, hsa-miR-6715b-5p, hsa-miR-6768-5p, hsa-miR-6867-5p, hsa-miR-7855-5p, hsa-miR-8485, and any combination thereof, may be administered to a pancreatic cancer subject to treat, ameliorate, and/or reduce the severity of pancreatic cancer or one or more symptoms thereof in the pancreatic cancer subject: (i) via a route selected from the group consisting of buccally, inhalation, intraarterially, intraarticularly, intracardially, intracerebroventricularly, intradermally, intramedullary, intramuscularly, intraperitoneally, intrathecally, intravenously, intraventricularly, nasally, ophthalmically, orally, rectally, sublabially, sublingually, subcutaneously, topically, and transdermally; and/or (ii) with a frequency selected from the group consisting of four times daily, three times daily, twice daily, once daily, once every two days, once every three days, once every four days, once every five days, once every six days, once a week, once every eight days, once every nine days, once every ten days, once every two weeks, once every three weeks, and once a month; and/or (iii) at a dosage (based on subject weight) selected from the group consisting of 0.1 μg/kg, 0.5 μg/kg, 1 μg/kg, 1.5 μg/kg, 2.0 μg/kg, 2.5 μg/kg, 3.0 μg/kg, 3.5 μg/kg, 4.0 μg/kg, 4.5 μg/kg, 5.0 μg/kg, 10 μg/kg, 15 μg/kg, 20 μg/kg, 25 μg/kg, 30 μg/kg, 35 μg/kg, 40 μg/kg, 45 μg/kg, 50 μg/kg, 55 μg/kg, 60 μg/kg, 65 μg/kg, 70 μg/kg, 75 μg/kg, 80 μg/kg, 85 μg/kg, 90 μg/kg, 95 μg/kg, 100 μg/kg, 150 μg/kg, 200 μg/kg, 250 μg/kg, 300 μg/kg, 350 μg/kg, 400 μg/kg, 450 μg/kg, 500 μg/kg, 550 μg/kg, 600 μg/kg, 650 μg/kg, 700 μg/kg, 750 μg/kg, 800 μg/kg, 850 μg/kg, 900 μg/kg, 1 mg/kg, 1.5 mg/kg, 2.0 mg/kg, 2.5 mg/kg, 3 mg/kg, 3.5 mg/kg, 4.0 mg/kg, 4.5 mg/kg, 5 mg/kg, 5.5 mg/kg, 6 mg/kg, 6.5 mg/kg, 7 mg/kg, 7.5 mg/kg, 8 mg/kg, 8.5 mg/kg, 9 mg/kg, 9.5 mg/kg, 10 mg/kg 10.5 mg/kg, 11 mg/kg, 11.5 mg/kg, 12 mg/kg, 12.5 mg/kg, 13 mg/kg, 13.5 mg/kg, 14 mg/kg, 14.5 mg/kg, 15 mg/kg, 16 mg/kg, 17 mg/kg, 18 mg/kg, 19 mg/kg, 20 mg/kg, 21 mg/kg, 22 mg/kg, 23 mg/kg, 24 mg/kg, 25 mg/kg, 30 mg/kg, 35 mg/kg, 40 mg/kg, 45 mg/kg, 50 mg/kg, 55 mg/kg, 60 mg/kg, 65 mg/kg, 70 mg/kg, 75 mg/kg, 80 mg/kg, 85 mg/kg, 90 mg/kg, 95 mg/kg, 100 mg/kg, 105 mg/kg, 110 mg/kg, 115 mg/kg, 120 mg/kg, 125 mg/kg, 130 mg/kg, 135 mg/kg, 140 mg/kg, 145 mg/kg, 150 mg/kg, 155 mg/kg, 160 mg/kg, 165 mg/kg, 170 mg/kg, 175 mg/kg, 180 mg/kg, 185 mg/kg, 190 mg/kg, 195 mg/kg, 200 mg/kg, and any fraction or integer in between any two of the preceding amounts.

In some embodiments, an nNOS inhibitor composition comprising one or more CRISPR/Cas systems, may be administered as a suitable dosage form, as described herein, to treat, ameliorate, and/or reduce the severity of pancreatic cancer or one or more symptoms thereof in a pancreatic cancer subject. In certain embodiments, an nNOS inhibitor composition comprising one or more CRISPR/Cas systems comprising an endonuclease selected from the group consisting of Cas1, Cas2, Cas3, Cas4, Cas5, Cas6, Cas7, Cas8a, Cas8b, Cas8c, Cas9, Cas10, Cas10d, Cas12, Cas12a, Cas12b, Cas12c, Cas12d, Cas12e, Cas12f, Cas12g, Cas12h, Cas12i, Cas12k, C2c4, C2c8, C2c9, Cas13, Cas13a, Cas13b, Cas13c, Cas13d, Cas13x.1, Cse1, Cse2, Csy1, Csy2, Csy3, GSU0054, Csm2, Cmr5, Csx10, Csx11, Csf1, and Csn2, may be administered as a suitable dosage form, as described herein, to treat, ameliorate, and/or reduce the severity of pancreatic cancer or one or more symptoms thereof in a pancreatic cancer subject. In some embodiments, an nNOS inhibitor composition comprising one or more CRISPR/Cas systems comprising an endonuclease selected from the group consisting of Cas1, Cas2, Cas3, Cas4, Cas5, Cas6, Cas7, Cas8a, Cas8b, Cas8c, Cas9, Cas10, Cas10d, Cas12, Cas12a, Cas12b, Cas12c, Cas12d, Cas12e, Cas12f, Cas12g, Cas12h, Cas12i, Cas12k, C2c4, C2c8, C2c9, Cas13, Cas13a, Cas13b, Cas13c, Cas13d, Cas13x.1, Cse1, Cse2, Csy1, Csy2, Csy3, GSU0054, Csm2, Cmr5, Csx10, Csx11, Csf1, and Csn2, may be administered to a pancreatic cancer subject to treat, ameliorate, and/or reduce the severity of pancreatic cancer or one or more symptoms thereof in the pancreatic cancer subject: (i) via a route selected from the group consisting of buccally, inhalation, intraarterially, intraarticularly, intracardially, intracerebroventricularly, intradermally, intramedullary, intramuscularly, intraperitoneally, intrathecally, intravenously, intraventricularly, nasally, ophthalmically, orally, rectally, sublabially, sublingually, subcutaneously, topically, and transdermally; and/or (ii) with a frequency selected from the group consisting of four times daily, three times daily, twice daily, once daily, once every two days, once every three days, once every four days, once every five days, once every six days, once a week, once every eight days, once every nine days, once every ten days, once every two weeks, once every three weeks, and once a month; and/or (iii) at a dosage (based on subject weight) selected from the group consisting of 0.1 μg/kg, 0.5 μg/kg, 1 μg/kg, 1.5 μg/kg, 2.0 μg/kg, 2.5 μg/kg, 3.0 μg/kg, 3.5 μg/kg, 4.0 μg/kg, 4.5 μg/kg, 5.0 μg/kg, 10 μg/kg, 15 μg/kg, 20 μg/kg, 25 μg/kg, 30 μg/kg, 35 μg/kg, 40 μg/kg, 45 μg/kg, 50 μg/kg, 55 μg/kg, 60 μg/kg, 65 μg/kg, 70 μg/kg, 75 μg/kg, 80 μg/kg, 85 μg/kg, 90 μg/kg, 95 μg/kg, 100 μg/kg, 150 μg/kg, 200 μg/kg, 250 μg/kg, 300 μg/kg, 350 μg/kg, 400 μg/kg, 450 μg/kg, 500 μg/kg, 550 μg/kg, 600 μg/kg, 650 μg/kg, 700 μg/kg, 750 μg/kg, 800 μg/kg, 850 μg/kg, 900 μg/kg, 1 mg/kg, 1.5 mg/kg, 2.0 mg/kg, 2.5 mg/kg, 3 mg/kg, 3.5 mg/kg, 4.0 mg/kg, 4.5 mg/kg, 5 mg/kg, 5.5 mg/kg, 6 mg/kg, 6.5 mg/kg, 7 mg/kg, 7.5 mg/kg, 8 mg/kg, 8.5 mg/kg, 9 mg/kg, 9.5 mg/kg, 10 mg/kg 10.5 mg/kg, 11 mg/kg, 11.5 mg/kg, 12 mg/kg, 12.5 mg/kg, 13 mg/kg, 13.5 mg/kg, 14 mg/kg, 14.5 mg/kg, 15 mg/kg, 16 mg/kg, 17 mg/kg, 18 mg/kg, 19 mg/kg, 20 mg/kg, 21 mg/kg, 22 mg/kg, 23 mg/kg, 24 mg/kg, 25 mg/kg, 30 mg/kg, 35 mg/kg, 40 mg/kg, 45 mg/kg, 50 mg/kg, 55 mg/kg, 60 mg/kg, 65 mg/kg, 70 mg/kg, 75 mg/kg, 80 mg/kg, 85 mg/kg, 90 mg/kg, 95 mg/kg, 100 mg/kg, 105 mg/kg, 110 mg/kg, 115 mg/kg, 120 mg/kg, 125 mg/kg, 130 mg/kg, 135 mg/kg, 140 mg/kg, 145 mg/kg, 150 mg/kg, 155 mg/kg, 160 mg/kg, 165 mg/kg, 170 mg/kg, 175 mg/kg, 180 mg/kg, 185 mg/kg, 190 mg/kg, 195 mg/kg, 200 mg/kg, and any fraction or integer in between any two of the preceding amounts.

In some embodiments, an nNOS inhibitor composition comprising one or more antibodies, may be administered as a suitable dosage form, as described herein, to treat, ameliorate, and/or reduce the severity of pancreatic cancer or one or more symptoms thereof in a pancreatic cancer subject. In certain embodiments, an nNOS inhibitor composition comprising one or more antibodies selected from the group consisting of 37-2800 (from ThermoFisher), EP1855Y (from abeam), EPR 24351-6 (from abeam), and any combination thereof, may be administered as a suitable dosage form, as described herein, to treat, ameliorate, and/or reduce the severity of pancreatic cancer or one or more symptoms thereof in a pancreatic cancer subject. In some embodiments, an nNOS inhibitor composition comprising one or more antibodies selected from the group consisting of 37-2800 (from ThermoFisher), EP1855Y (from abeam), EPR 24351-6 (from abeam), and any combination thereof, may be administered to a pancreatic cancer subject to treat, ameliorate, and/or reduce the severity of pancreatic cancer or one or more symptoms thereof in the pancreatic cancer subject: (i) via a route selected from the group consisting of buccally, inhalation, intraarterially, intraarticularly, intracardially, intracerebroventricularly, intradermally, intramedullary, intramuscularly, intraperitoneally, intrathecally, intravenously, intraventricularly, nasally, ophthalmically, orally, rectally, sublabially, sublingually, subcutaneously, topically, and transdermally; and/or (ii) with a frequency selected from the group consisting of four times daily, three times daily, twice daily, once daily, once every two days, once every three days, once every four days, once every five days, once every six days, once a week, once every eight days, once every nine days, once every ten days, once every two weeks, once every three weeks, and once a month; and/or (iii) at a dosage (based on subject weight) selected from the group consisting of 0.1 μg/kg, 0.5 μg/kg, 1 μg/kg, 1.5 μg/kg, 2.0 μg/kg, 2.5 μg/kg, 3.0 μg/kg, 3.5 μg/kg, 4.0 μg/kg, 4.5 μg/kg, 5.0 μg/kg, 10 μg/kg, 15 μg/kg, 20 μg/kg, 25 μg/kg, 30 μg/kg, 35 μg/kg, 40 μg/kg, 45 μg/kg, 50 μg/kg, 55 μg/kg, 60 μg/kg, 65 μg/kg, 70 μg/kg, 75 μg/kg, 80 μg/kg, 85 μg/kg, 90 μg/kg, 95 μg/kg, 100 μg/kg, 150 μg/kg, 200 μg/kg, 250 μg/kg, 300 μg/kg, 350 μg/kg, 400 μg/kg, 450 μg/kg, 500 μg/kg, 550 μg/kg, 600 μg/kg, 650 μg/kg, 700 μg/kg, 750 μg/kg, 800 μg/kg, 850 μg/kg, 900 μg/kg, 1 mg/kg, 1.5 mg/kg, 2.0 mg/kg, 2.5 mg/kg, 3 mg/kg, 3.5 mg/kg, 4.0 mg/kg, 4.5 mg/kg, 5 mg/kg, 5.5 mg/kg, 6 mg/kg, 6.5 mg/kg, 7 mg/kg, 7.5 mg/kg, 8 mg/kg, 8.5 mg/kg, 9 mg/kg, 9.5 mg/kg, 10 mg/kg 10.5 mg/kg, 11 mg/kg, 11.5 mg/kg, 12 mg/kg, 12.5 mg/kg, 13 mg/kg, 13.5 mg/kg, 14 mg/kg, 14.5 mg/kg, 15 mg/kg, 16 mg/kg, 17 mg/kg, 18 mg/kg, 19 mg/kg, 20 mg/kg, 21 mg/kg, 22 mg/kg, 23 mg/kg, 24 mg/kg, 25 mg/kg, 30 mg/kg, 35 mg/kg, 40 mg/kg, 45 mg/kg, 50 mg/kg, 55 mg/kg, 60 mg/kg, 65 mg/kg, 70 mg/kg, 75 mg/kg, 80 mg/kg, 85 mg/kg, 90 mg/kg, 95 mg/kg, 100 mg/kg, 105 mg/kg, 110 mg/kg, 115 mg/kg, 120 mg/kg, 125 mg/kg, 130 mg/kg, 135 mg/kg, 140 mg/kg, 145 mg/kg, 150 mg/kg, 155 mg/kg, 160 mg/kg, 165 mg/kg, 170 mg/kg, 175 mg/kg, 180 mg/kg, 185 mg/kg, 190 mg/kg, 195 mg/kg, 200 mg/kg, and any fraction or integer in between any two of the preceding amounts.

In some embodiments, an nNOS inhibitor composition comprising one or more amino acid-based compounds, may be administered as a suitable dosage form, as described herein, to treat, ameliorate, and/or reduce the severity of pancreatic cancer or one or more symptoms thereof in a pancreatic cancer subject. In certain embodiments, an nNOS inhibitor composition comprising one or more amino acid-compounds that bind an active site of nNOS selected from the group consisting of Phenylalanine 589, Tryptophan 592, Tyrosine 593, Glutamate 597, Aspartate 602, Arginine 601, Methionine 341, Histidine 342, and Tyrosine711, may be administered as a suitable dosage form, as described herein, to treat, ameliorate, and/or reduce the severity of pancreatic cancer or one or more symptoms thereof in a pancreatic cancer subject. In some embodiments, an nNOS inhibitor composition comprising one or more amino acid-compounds that bind an active site of nNOS selected from the group consisting of Phenylalanine 589, Tryptophan 592, Tyrosine 593, Glutamate 597, Aspartate 602, Arginine 601, Methionine 341, Histidine 342, and Tyrosine711, may be administered to a pancreatic cancer subject to treat, ameliorate, and/or reduce the severity of pancreatic cancer or one or more symptoms thereof in the pancreatic cancer subject: (i) via a route selected from the group consisting of buccally, inhalation, intraarterially, intraarticularly, intracardially, intracerebroventricularly, intradermally, intramedullary, intramuscularly, intraperitoneally, intrathecally, intravenously, intraventricularly, nasally, ophthalmically, orally, rectally, sublabially, sublingually, subcutaneously, topically, and transdermally; and/or (ii) with a frequency selected from the group consisting of four times daily, three times daily, twice daily, once daily, once every two days, once every three days, once every four days, once every five days, once every six days, once a week, once every eight days, once every nine days, once every ten days, once every two weeks, once every three weeks, and once a month; and/or (iii) at a dosage (based on subject weight) selected from the group consisting of 0.1 μg/kg, 0.5 μg/kg, 1 μg/kg, 1.5 μg/kg, 2.0 μg/kg, 2.5 μg/kg, 3.0 μg/kg, 3.5 μg/kg, 4.0 μg/kg, 4.5 μg/kg, 5.0 μg/kg, 10 μg/kg, 15 μg/kg, 20 μg/kg, 25 μg/kg, 30 μg/kg, 35 μg/kg, 40 μg/kg, 45 μg/kg, 50 μg/kg, 55 μg/kg, 60 μg/kg, 65 μg/kg, 70 μg/kg, 75 μg/kg, 80 μg/kg, 85 μg/kg, 90 μg/kg, 95 μg/kg, 100 μg/kg, 150 μg/kg, 200 μg/kg, 250 μg/kg, 300 μg/kg, 350 μg/kg, 400 μg/kg, 450 μg/kg, 500 μg/kg, 550 μg/kg, 600 μg/kg, 650 μg/kg, 700 μg/kg, 750 μg/kg, 800 μg/kg, 850 μg/kg, 900 μg/kg, 1 mg/kg, 1.5 mg/kg, 2.0 mg/kg, 2.5 mg/kg, 3 mg/kg, 3.5 mg/kg, 4.0 mg/kg, 4.5 mg/kg, 5 mg/kg, 5.5 mg/kg, 6 mg/kg, 6.5 mg/kg, 7 mg/kg, 7.5 mg/kg, 8 mg/kg, 8.5 mg/kg, 9 mg/kg, 9.5 mg/kg, 10 mg/kg 10.5 mg/kg, 11 mg/kg, 11.5 mg/kg, 12 mg/kg, 12.5 mg/kg, 13 mg/kg, 13.5 mg/kg, 14 mg/kg, 14.5 mg/kg, 15 mg/kg, 16 mg/kg, 17 mg/kg, 18 mg/kg, 19 mg/kg, 20 mg/kg, 21 mg/kg, 22 mg/kg, 23 mg/kg, 24 mg/kg, 25 mg/kg, 30 mg/kg, 35 mg/kg, 40 mg/kg, 45 mg/kg, 50 mg/kg, 55 mg/kg, 60 mg/kg, 65 mg/kg, 70 mg/kg, 75 mg/kg, 80 mg/kg, 85 mg/kg, 90 mg/kg, 95 mg/kg, 100 mg/kg, 105 mg/kg, 110 mg/kg, 115 mg/kg, 120 mg/kg, 125 mg/kg, 130 mg/kg, 135 mg/kg, 140 mg/kg, 145 mg/kg, 150 mg/kg, 155 mg/kg, 160 mg/kg, 165 mg/kg, 170 mg/kg, 175 mg/kg, 180 mg/kg, 185 mg/kg, 190 mg/kg, 195 mg/kg, 200 mg/kg, and any fraction or integer in between any two of the preceding amounts.

In some embodiments, a pancreatic cancer subject, as described herein, may be a female child or a male child. In certain embodiments, a pancreatic cancer subject, as described herein, may be a female adult or a male adult.

In certain embodiments, a pancreatic cancer subject, as described herein, may have a pancreatic cancer selected from the group consisting of an acinar cell carcinoma, an adenocarcinoma, an adenosquamous carcinoma, a colloid carcinoma, a gastrinoma, a giant cell tumor, a glucagonoma, a hepatoid carcinoma, an insulinoma, an intraductal papillary-mucinous neoplasm, a lymphoma, a mucinous cystic neoplasm, a mucinous cystic neoplasm with an invasive adenocarcinoma, a nonfunctional islet cell tumor, a pancreatic intraepithelial lesion, a pancreatic neuroendocrine tumor (PNET), a pancreatoblastoma, a PPoma, a sarcoma, a serous cystadenoma, a signet ring cell carcinoma, a solid pseudopapillary neoplasm, a somatostatinoma, a squamous cell carcinoma, an undifferentiated carcinoma, and a VIPoma.

In some embodiments, a pancreatic cancer subject, as described herein, may have an abnormal expression of one or more antigens compared to an equivalent subject that does not have pancreatic cancer. As used herein, the term “equivalent subject,” refers to a subject of the same sex (male/female) and age range (child/adult) of the pancreatic cancer subject. For purposes of disclosure, the term “antigen,” when used with respect to abnormal expression, may define one or more CD antigens, such as, for example, CD15, CD44, CD70, CD133, as well as any CD antigen that has an abnormal expression in a pancreatic cancer disclosed herein that would be readily envisaged by one skilled in the art. In certain embodiments, abnormal expression of an antigen may define an abnormal expression of CD15, an abnormal expression of CD44, an abnormal expression of CD70, an abnormal expression of CD133, an abnormal expression of a CD antigen that has an abnormal expression in a pancreatic cancer disclosed herein that would be readily envisaged by one skilled in the art, and any combination thereof. In some embodiments, an “abnormal expression,” can refer to a reduced expression of an antigen, as described herein. In certain embodiments, an “abnormal expression,” can refer to an overexpression of an antigen, as described herein. Whether “abnormal expression,” refers to a reduced expression of an antigen or an overexpression of an antigen would be immediately envisaged by the skilled artisan when viewing the term in the context of the particular antigen disclosed, as well as the pancreatic cancer to which the abnormal expression of antigen refers to. For example, in the context of CD70 and pancreatic carcinomas, one skilled in the art would immediately envisage that “abnormal expression” refers to an overexpression of CD70. Treatment, amelioration, and/or reduction in the severity of a pancreatic cancer or one or more symptoms thereof may be characterized by a restoration to a normal level of expression of one or more antigens from their abnormal state, as described herein. For example, in the context of CD70 and pancreatic carcinomas, treatment, amelioration, and/or reduction in the severity of a pancreatic cancer or one or more symptoms thereof may be characterized by a reduced expression of CD70.

In certain embodiments, a pancreatic cancer subject, as described herein, may have an abnormal expression of one or more proteins compared to an equivalent subject that does not have pancreatic cancer. For purposes of disclosure, the term “protein,” when used with respect to abnormal expression, may define one or more proteins and/or enzymes, such as, for example, ALDH1A3, mTOR, p-mTOR, TSC-2, AKT, pAKT, RPS6, p-RPS6, Syp, VEGF, PIGF, as well as any protein or enzyme that has an abnormal expression in a pancreatic cancer disclosed herein that would be readily envisaged by one skilled in the art. In certain embodiments, abnormal expression of a protein may define an abnormal expression of ALDH1A3, an abnormal expression of mTOR, an abnormal expression of p-mTOR, an abnormal expression of TSC-2, an abnormal expression of AKT, an abnormal expression of pAKT, an abnormal expression of RPS6, an abnormal expression of p-RPS6, an abnormal expression of Syp, an abnormal expression of VEGF, an abnormal expression of PIGF, an abnormal expression of a protein or enzyme that has an abnormal expression in a pancreatic cancer disclosed herein that would be readily envisaged by one skilled in the art, and any combination thereof. In some embodiments, an “abnormal expression,” can refer to a reduced expression of a protein, as described herein. In certain embodiments, an “abnormal expression,” can refer to an overexpression of a protein, as described herein. Whether “abnormal expression,” refers to a reduced expression of a protein or an overexpression of a protein would be immediately envisaged by the skilled artisan when viewing the term in the context of the particular protein disclosed, as well as the pancreatic cancer to which the abnormal expression of protein refers to. For example, in the context of VEGF and pancreatic ductal adenocarcinoma (“PDAC”), one skilled in the art would immediately envisage that “abnormal expression” refers to an overexpression of VEGF. Treatment, amelioration, and/or reduction in the severity of a pancreatic cancer or one or more symptoms thereof may be characterized by a restoration to a normal level of expression of one or more proteins from their abnormal state, as described herein. For example, in the context of VEGF and PDAC, treatment, amelioration, and/or reduction in the severity of a pancreatic cancer or one or more symptoms thereof may be characterized by a reduced expression of VEGF.

In certain embodiments, a pancreatic cancer subject, as described herein, may have an increased expression of one or more NOS isoforms compared to an equivalent subject that does not have pancreatic cancer. For purposes of disclosure, the term “NOS isoform,” refers to nNOS, iNOS, and/or eNOS. In certain embodiments, a pancreatic cancer subject, as described herein, may have an increased expression of nNOS compared to an equivalent subject that does not have pancreatic cancer. In some embodiments, a pancreatic cancer subject, as described herein, may have an increased expression of iNOS compared to an equivalent subject that does not have pancreatic cancer. In certain embodiments, a pancreatic cancer subject, as described herein, may have an increased expression of eNOS compared to an equivalent subject that does not have pancreatic cancer. In some embodiments, a pancreatic cancer subject, as described herein, may have an increased expression of nNOS and iNOS compared to an equivalent subject that does not have pancreatic cancer. In certain embodiments, a pancreatic cancer subject, as described herein, may have an increased expression of nNOS and eNOS compared to an equivalent subject that does not have pancreatic cancer. In some embodiments, a pancreatic cancer subject, as described herein, may have an increased expression of iNOS and eNOS compared to an equivalent subject that does not have pancreatic cancer. In certain embodiments, a pancreatic cancer subject, as described herein, may have an increased expression of nNOS, iNOS, and eNOS compared to an equivalent subject that does not have pancreatic cancer. Whether a “NOS isoform,” has an increased expression would be immediately envisaged by the skilled artisan when viewing the term in the context of the particular NOS isoform disclosed, as well as the pancreatic cancer to which the increased expression of NOS isoform refers to. For example, in the context of PDAC, one skilled in the art would immediately envisage that eNOS and/or iNOS have an increased expression. Treatment, amelioration, and/or reduction in the severity of a pancreatic cancer or one or more symptoms thereof may be characterized by a reduced expression of one or more NOS isoforms from their increased state, as described herein. For example, in the context of PDAC, treatment, amelioration, and/or reduction in the severity of a pancreatic cancer or one or more symptoms thereof may be characterized by a reduced expression of eNOS and/or iNOS. For purposes of disclosure, reduction/inhibition of the expression of one or more NOS isoforms may be characterized by a reduction in expression of one or more NOS isoforms by about at least 10%, about at least 20%, about at least 30%, or about at least 40%. In some embodiments, reduction/inhibition of the expression of one or more NOS isoforms may further be characterized by a lack reduction of other, non-implicated NOS isoforms, such as, for example, no more than about 10%, no more than about 5%, no more than about 2%, or no more than about 1% reduction/inhibition of the expression of non-implicated NOS isoforms. As used herein, the term “non-implicated NOS isoforms,” refers to NOS isoforms that do not have an increased expression in a particular pancreatic cancer. By way of example, in the context of PDAC, if both eNOS and iNOS have an increased expression, nNOS would define a “non-implicated” NOS isoform. Whether a NOS isoform is a “non-implicated NOS isoform,” would be immediately envisaged by the skilled artisan when viewing the term in the context of the particular NOS isoform disclosed, as well as the pancreatic cancer.

In some embodiments, a pancreatic cancer subject, as described herein, may have an elevated level of one or more NO-indicators compared to an equivalent subject that does not have pancreatic cancer. As used herein, the term “NO-indicator,” can refer to 3-nitrotyrosine (3-Ntyr), NADPH-diaphorase, total nitrite concentration, total nitrate concentration, total nitrite and nitrate concentration, as well as any NO-indicator that has an elevated level in a pancreatic cancer disclosed herein that would be readily envisaged by one skilled in the art. For example, in the context of pancreatic adenocarcinoma, one skilled in the art would immediately envisage that 3-Ntyr can be present at an elevated level. Treatment, amelioration, and/or reduction in the severity of a pancreatic cancer or one or more symptoms thereof may be characterized by a reduction in the level of one or more NO-indicators. For example, in the context of pancreatic adenocarcinoma, treatment, amelioration, and/or reduction in the severity of a pancreatic cancer or one or more symptoms thereof may be characterized by a reduction in the level of 3-Ntyr.

In certain embodiments, a NOS inhibitor composition, as described herein, may be administered as a suitable dosage form, as described herein, to treat, ameliorate, and/or reduce the severity of a pancreatic cancer or one or more symptoms thereof, as an alternative to a conventional therapy for treating, ameliorating, and/or reducing the severity of a pancreatic cancer or one or more symptoms thereof. For purposes of disclosure, a “conventional therapy,” as described herein, may refer to a treatment that would be recommended by one skilled in the art to treat a pancreatic cancer, and may define one or more of radiation, chemotherapy, immunotherapy, cell-based therapy, gene therapy, and the like—one skilled in the art would immediately envisage a “conventional therapy” based on the particular pancreatic cancer disclosed, as well as characteristics of a pancreatic cancer subject, such as, for example, the subject's sex, age, stage of the pancreatic cancer, genotype of the pancreatic cancer, and the like. Treatment, amelioration, and/or reduction in the severity of a pancreatic cancer or one or more symptoms thereof may be characterized by an improved outcome compared to a conventional therapy, as described herein. For purposes of the disclosure, an “improved outcome” may define any one of the following: (i) a larger reduction in tumor volume and/or mass compared to a subject administered an equivalent dose of a conventional therapy; (ii) an improvement in the expected rate of survival of a subject, at, for example, 1 month, 3 months, 6 months, 9 months, 1 year, 2 years, 3 years, 5 years, 10 years, or more, compared to the expected rate of survival of a subject administered an equivalent dose of a conventional therapy; (iii) a reduction in the prevalence of side effects resulting from the NOS inhibitor composition compared to a subject administered an equivalent dose of a conventional therapy; (iv) a reduction in the severity of side effects resulting from the NOS inhibitor composition compared to a subject administered an equivalent dose of a conventional therapy; (v) a reduction in the dosage required to achieve an equivalent reduction in tumor volume compared to a subject administered a conventional therapy; (vi) a reduction in the dosage required to achieve an equivalent expected rate of survival of a subject compared to a subject administered a conventional therapy; (vii) a reduction in the likelihood of a recurrence of the pancreatic cancer occurring in a subject compared to a subject administered an equivalent dose of a conventional therapy; (viii) a reduction in the likelihood of a metastasis occurring in a subject compared to a subject administered an equivalent dose of a conventional therapy; (ix) a reduction in the time required to be at a medical facility for treatment compared to a subject administered an equivalent dose of a conventional therapy; (x) a reduction in the cost associated with treatment compared to a subject administered an equivalent dose of a conventional therapy; as well as any other improved outcome not expressly recited that would be readily envisaged by one skilled in the art.

In some embodiments, a NOS inhibitor composition, as described herein, may be administered as a suitable dosage form, as described herein, to treat, ameliorate, and/or reduce the severity of a pancreatic cancer or one or more symptoms thereof, wherein the NOS inhibitor composition comprises a first chemical NOS inhibitor and a second chemical NOS inhibitor. In certain embodiments, the first chemical NOS inhibitor may be administered concurrently with the second chemical NOS inhibitor. In some embodiments, the first chemical NOS inhibitor may be administered prior to the second chemical NOS inhibitor. In certain embodiments, the first chemical NOS inhibitor may be administered subsequently to the second chemical NOS inhibitor. Treatment, amelioration, and/or reduction in the severity of a pancreatic cancer or one or more symptoms thereof may be characterized by an improved outcome compared an alternative treatment. As used herein, the term “alternative treatment,” may define one or more of a conventional therapy, as described herein, a NOS inhibitor composition comprising a single chemical NOS inhibitor, as described herein, and a NOS inhibitor composition comprising a single biological NOS inhibitor, as described herein. For purposes of the disclosure, an “improved outcome” may define any one of the following: (i) a larger reduction in tumor volume and/or mass compared an alternative treatment; (ii) an improvement in the expected rate of survival of a subject, at, for example, 1 month, 3 months, 6 months, 9 months, 1 year, 2 years, 3 years, 5 years, 10 years, or more, compared to the expected rate of survival of a subject administered an alternative treatment; (iii) a reduction in the prevalence of side effects resulting from the NOS inhibitor composition comprising at least two chemical NOS inhibitors compared to a subject administered an alternative treatment; (iv) a reduction in the severity of side effects resulting from the NOS inhibitor composition comprising at least two chemical NOS inhibitors compared to a subject administered an alternative treatment; (v) a reduction in the dosage required to achieve an equivalent reduction in tumor volume compared to a subject administered an alternative treatment; (vi) a reduction in the dosage required to achieve an equivalent expected rate of survival of a subject compared to a subject administered an alternative treatment; (vii) a reduction in the likelihood of a recurrence of the pancreatic cancer in a subject compared to a subject administered an alternative treatment; (viii) a reduction in the likelihood of a metastasis occurring in a subject compared to a subject administered an alternative treatment; (ix) a reduction in the time required to be at a medical facility for treatment compared to a subject administered an alternative treatment; (x) a reduction in the cost associated with treatment compared to a subject administered an alternative treatment; as well as any other improved outcome not expressly recited that would be readily envisaged by one skilled in the art.

In certain embodiments, a NOS inhibitor composition, as described herein, may be used to treat, ameliorate, and/or reduce the severity of a pancreatic cancer or one or more symptoms thereof, wherein the NOS inhibitor composition comprises at least one chemical NOS inhibitor and at least one biological NOS inhibitor. In some embodiments, the at least one chemical NOS inhibitor may be administered concurrently with the at least one biological NOS inhibitor. In certain embodiments, the at least one chemical NOS inhibitor may be administered prior to the at least one biological NOS inhibitor. In some embodiments, the at least one chemical NOS inhibitor may be administered subsequently to the at least one biological NOS inhibitor. Treatment, amelioration, and/or reduction in the severity of a pancreatic cancer or one or more symptoms thereof may be characterized by an improved outcome compared an alternative treatment. As used herein, the term “alternative treatment,” may define one or more of a conventional therapy, as described herein, a NOS inhibitor composition comprising a single chemical NOS inhibitor, as described herein, and a NOS inhibitor composition comprising a single biological NOS inhibitor, as described herein. For purposes of the disclosure, an “improved outcome” may define any one of the following: (i) a larger reduction in tumor volume and/or mass compared an alternative treatment; (ii) an improvement in the expected rate of survival of a subject, at, for example, 1 month, 3 months, 6 months, 9 months, 1 year, 2 years, 3 years, 5 years, 10 years, or more, compared to the expected rate of survival of a subject administered an alternative treatment; (iii) a reduction in the prevalence of side effects resulting from the NOS inhibitor composition comprising at least one chemical NOS inhibitor and at least one biological NOS inhibitor compared to a subject administered an alternative treatment; (iv) a reduction in the severity of side effects resulting from the NOS inhibitor composition comprising at least one chemical NOS inhibitor and at least one biological NOS inhibitor compared to a subject administered an alternative treatment; (v) a reduction in the dosage required to achieve an equivalent reduction in tumor volume compared to a subject administered an alternative treatment; (vi) a reduction in the dosage required to achieve an equivalent expected rate of survival of a subject compared to a subject administered an alternative treatment; (vii) a reduction in the likelihood of a recurrence of the pancreatic cancer in a subject compared to a subject administered an alternative treatment; (viii) a reduction in the likelihood of a metastasis occurring in a subject compared to a subject administered an alternative treatment; (ix) a reduction in the time required to be at a medical facility for treatment compared to a subject administered an alternative treatment; (x) a reduction in the cost associated with treatment compared to a subject administered an alternative treatment; as well as any other improved outcome not expressly recited that would be readily envisaged by one skilled in the art.

In some embodiments, a NOS inhibitor composition, as described herein, may be combined with a conventional therapy, as described herein, to treat, ameliorate, and/or reduce the severity of a pancreatic cancer or one or more symptoms thereof. In certain embodiments, the NOS inhibitor composition may be administered concurrently with the conventional therapy. In some embodiments, the NOS inhibitor composition may be administered prior to the conventional therapy. In certain embodiments, the NOS inhibitor composition may be administered subsequently to the conventional therapy. Treatment, amelioration, and/or reduction in the severity of a pancreatic cancer or one or more symptoms thereof may be characterized by an improved outcome compared an alternative treatment. As used herein, the term “alternative treatment,” may define one or more of a conventional therapy, as described herein, a NOS inhibitor composition comprising a single chemical NOS inhibitor, as described herein, and a NOS inhibitor composition comprising a single biological NOS inhibitor, as described herein. For purposes of the disclosure, an “improved outcome” may define any one of the following: (i) a larger reduction in tumor volume and/or mass compared an alternative treatment; (ii) an improvement in the expected rate of survival of a subject, at, for example, 1 month, 3 months, 6 months, 9 months, 1 year, 2 years, 3 years, 5 years, 10 years, or more, compared to the expected rate of survival of a subject administered an alternative treatment; (iii) a reduction in the prevalence of side effects resulting from the NOS inhibitor composition and conventional therapy compared to a subject administered an alternative treatment; (iv) a reduction in the severity of side effects resulting from the NOS inhibitor composition and conventional therapy compared to a subject administered an alternative treatment; (v) a reduction in the dosage required to achieve an equivalent reduction in tumor volume compared to a subject administered an alternative treatment; (vi) a reduction in the dosage required to achieve an equivalent expected rate of survival of a subject compared to a subject administered an alternative treatment; (vii) a reduction in the likelihood of a recurrence of the pancreatic cancer in a subject compared to a subject administered an alternative treatment; (viii) a reduction in the likelihood of a metastasis occurring in a subject compared to a subject administered an alternative treatment; (ix) a reduction in the time required to be at a medical facility for treatment compared to a subject administered an alternative treatment; (x) a reduction in the cost associated with treatment compared to a subject administered an alternative treatment; as well as any other improved outcome not expressly recited that would be readily envisaged by one skilled in the art.

In some embodiments, a “conventional therapy,” as that term is used with respect to combined administration with a NOS inhibitor composition, may refer to a conventional chemotherapy. For purposes of the disclosure, a “conventional chemotherapy,” as described herein may be a compound selected from the group consisting of abemaciclib, abiraterone acetate, acalabrutinib, adagrasib, afatinib dimaleate, albumin-bound paclitaxel, albumin-bound rapamycin (Sirolimus Protein-Bound Particles), alectinib, alpelisib, amifostine, aminolevulinic acid hydrochloride, anastrozole, apalutamide, aprepitant, arsenic trioxide, asciminib hydrochloride, avapritinib, axitinib, azacytidine, belinostat, belzutifan, bendamustine hydrochloride, bexarotene, bicalutamide, binimetinib, bleomycin, bleomycin sulfate, bortezomib, bosutinib, brigatinib, busulfan, cabazitaxel, cabozantinib-S-malate, capecitabine, capmatinib hydrochloride, carboplatin, carfilzomib, carmustine, cedazuridine, ceritinib, chlorambucil, cisplatin, cladribine, clofarabine, cobimetinib fumarate, copanlisib hydrochloride, crizotinib, cyclophosphamide, cytarabine, dabrafenib mesylate, dacarbazine, dacomitinib, dactinomycin, darolutamide, dasatinib, daunorubicin hydrochloride, decitabine, defibrotide sodium, degarelix, dexamethasone, dexrazoxane hydrochloride, docetaxel, doxorubicin hydrochloride, duvelisib, elacestrant dihydrochloride, eltrombopag olamine, enasidenib mesylate, encorafenib, entrectinib, enzalutamide, epirubicin hydrochloride, erdafitinib, eribulin mesylate, erlotinib hydrochloride, etoposide, etoposide phosphate, everolimus, exemestane, fedratinib hydrochloride, fludarabine phosphate, fluorouracil, flutamide, folinic acid, fostamatinib disodium, fulvestrant, futibatinib, gefitinib, gemcitabine hydrochloride, gilteritinib fumarate, glasdegib maleate, goserelin acetate, granisetron, granisetron hydrochloride, hydroxyurea, ibrutinib, idarubicin hydrochloride, idelalisib, ifosfamide, imatinib mesylate, imiquimod, infigratinib phosphate, iobenguane I-131, irinotecan hydrochloride, ivosidenib, ixabepilone, ixazomib citrate, lanreotide acetate, lapatinib ditosylate, larotrectinib sulfate, lenalidomide, lenvatinib mesylate, letrozole, leucovorin calcium, leuprolide acetate, liposomal cytarabine, liposomal doxorubicin hydrochloride, liposomal irinotecan hydrochloride, lomustine, lorlatinib, lurbinectedin, Lu-177 dotatate, Lu-177 vipivotide tetraxetan, mechlorethamine hydrochloride, megestrol acetate, melphalan, melphalan hydrochloride, mercaptopurine, mesna, methotrexate, methotrexate sodium, methylnaltrexone bromide, midostaurin, mitomycin, mitoxantrone hydrochloride, mobocertinib succinate, nelarabine, neratinib maleate, netupitant hydrochloride, nilutamide, nilotinib, niraparib tosylate monohydrate, olaparib, olutasidenib, omacetaxine mepesuccinate, ondansetron hydrochloride, osimertinib mesylate, oxaliplatin, paclitaxel, pacritinib citrate, palbociclib, palonosetron hydrochloride, pamidronate disodium, pazopanib hydrochloride, pemetrexed disodium, pemigatinib, pexidartinib hydrochloride, pirtobrutinib, plerixafor, pomalidomide, ponatinib hydrochloride, pralatrexate, pralsetinib, prednisone, procarbazine hydrochloride, propranolol hydrochloride, Ra-223 dichloride, raloxifene hydrochloride, regorafenib, relugolix, ribociclib, ripretinib, rolapitant hydrochloride, romidepsin, rucaparib camsylate, ruxolitinib phosphate, selinexor, selpercatinib, selumetinib sulfate, sonidegib, sorafenib tosylate, sotorasib, sunitinib malate, talazoparib tosylate, talc, tamoxifen citrate, tazemetostat hydrobromide, temozolomide, temsirolimus, tepotinib hydrochloride, thalidomide, thioguanine, thiotepa, tipiracil hydrochloride, tivozanib hydrochloride, topotecan hydrochloride, toremifene, trabectedin, trametinib dimethyl sulfoxide, tretinoin, trifluridine hydrochloride, tucatinib, uridine triacetate, valrubicin, vandetanib, vemurafenib, venetoclax, vinblastine sulfate, vincristine sulfate, vinorelbine tartrate, vismodegib, vorinostat, zanubrutinib, zoledronic acid, and any combination thereof. One skilled in the art would immediately envisage an appropriate “conventional chemotherapy” based on the particular pancreatic cancer disclosed, as well as characteristics of a pancreatic cancer subject, such as, for example, the subject's sex, age, stage of the pancreatic cancer, genotype of the pancreatic cancer, and the like.

In certain embodiments, a “conventional therapy,” as that term is used with respect to combined administration with a NOS inhibitor composition, may refer to a conventional immunotherapy. For purposes of the disclosure, a “conventional immunotherapy,” as described herein may be an immunotherapy, cell-based therapy, and/or gene-based therapy selected from the group consisting of ado-trastuzumab emtansine, aldesleukin, alemtuzumab, amivantamab-vmjw, asparaginase Erwinia chrysanthemi, asparaginase Erwinia chrysanthemi (recombinant)-rywn, atezolizumab, avelumab, axicabtagene ciloleucel, bevacizumab, blinatumomab, brentuximab vedotin, brexucabtagene autoleucel, calaspargase pegol-mknl, camrelizumab, caplacizumab-yhdp, cemiplimab-rwlc, cetuximab, ciltacabtagene autoleucel, daratumumab, daratumumab and hyaluronidase-fihj, darbepoetin alfa, denileukin diftitox, denosumab, dinutuximab, dostarlimab-gxly, durvalumab, elotuzumab, emapalumab-lzsg, enfortumab vedotin-ejfv, epoetin alfa, fam-trastuzumab deruxtecan-nxki, filgrastim, gemtuzumab ozogamicin, glucarpidase, ibritumomab tiuxetan, idecabtagene vicleucel, inotuzumab ozogamicin, ipilimumab, isatuximab-irfc, lisocabtagene maraleucel, loncastuximab tesirine-lpyl, luspatercept-aamt, margetuximab-cmkb, mirvetuximab soravtansine-gynx, mogamulizumab-kpkc, mosunetuzumab-axgb, moxetumomab pasudotox-tdfk, nadofaragene firadenovec-vncg, naxitamab-gqgk, necitumumab, nivolumab, nivolumab and relatlimab-rmbw, obinutuzumab, ofatumumab, palifermin, panitumumab, pegaspargase, pegfilgrastim, pembrolizumab, pertuzumab, pertuzumab, trastuzumab, and hyaluronidase-zzxf, polatuzumab vedotin-piiq, ramucirumab, rasburicase, ravulizumab-cwvz, recombinant HPV bivalent vaccine, recombinant HPV nonavalent vaccine, recombinant HPV quadrivalent vaccine, recombinant interferon alfa-2b, rituximab, rituximab and hyaluronidase human, romiplostim, ropeginterferon alfa-2b-njft, sacituzumab govitecan-hziy, siltuximab, sintilimab, sipuleucel-T, tafasitamab-cxix, tagraxofusp-erzs, talimogene laherparepvec, tebentafusp-tebn, teclistamab-cqyv, tisagenlecleucel, tislelizumab, tisotumab vedotin-tftv, tocilizumab, toripalimab, trastuzumab, trastuzumab and hyaluronidase-oysk, tremelimumab-actl, ziv-aflibercept, and any combination thereof. One skilled in the art would immediately envisage an appropriate “conventional immunotherapy” based on the particular pancreatic cancer disclosed, as well as characteristics of a pancreatic cancer subject, such as, for example, the subject's sex, age, stage of the pancreatic cancer, genotype of the pancreatic cancer, and the like.

In certain embodiments, a NOS inhibitor composition, as described herein, may be administered as a suitable dosage form, as described herein, prior to, or alternatively, following a surgery to remove or resect a pancreatic cancer.

In some embodiments, a NOS inhibitor composition, as described herein, may be administered as a suitable dosage form, as described herein, to restore and/or maintain healthy and/or optimal expression levels of one or more antigens, as described herein, such as, for example, CD15, CD44, CD70, CD133, as well as any CD antigen that has an abnormal expression in a pancreatic cancer disclosed herein that would be readily envisaged by one skilled in the art, in a subject. In certain embodiments, the subject may have a pancreatic cancer or one or more symptoms thereof. As used herein, the terms “healthy” and “optimal” refer to expression levels of one or more antigens that would be typical of an equivalent subject that does not have pancreatic cancer.

In certain embodiments, a NOS inhibitor composition, as described herein, may be administered as a suitable dosage form, as described herein, to restore and/or maintain healthy and/or optimal expression levels of one or more proteins, as described herein, such as, for example, ALDH1A3, mTOR, p-mTOR, TSC-2, AKT, pAKT, RPS6, p-RPS6, Syp, VEGF, PIGF, as well as any protein or enzyme that has an abnormal expression in a pancreatic cancer disclosed herein that would be readily envisaged by one skilled in the art, in a subject. In certain embodiments, the subject may have a pancreatic cancer or one or more symptoms thereof. As used herein, the terms “healthy” and “optimal” refer to expression levels of one or more proteins that would be typical of an equivalent subject that does not have pancreatic cancer.

In some embodiments, a NOS inhibitor composition, as described herein, may be administered as a suitable dosage form, as described herein, to restore and/or maintain healthy and/or optimal expression levels of one or more NOS isoforms, as described herein, such as, for example, nNOS, iNOS, and eNOS, in a subject. In certain embodiments, the subject may have a pancreatic cancer or one or more symptoms thereof. As used herein, the terms “healthy” and “optimal” refer to expression levels of one or more NOS isoforms that would be typical of an equivalent subject that does not have pancreatic cancer.

In certain embodiments, a NOS inhibitor composition, as described herein, may be administered as a suitable dosage form, as described herein, to restore and/or maintain healthy and/or optimal levels of one or more NO-indicators, as described herein, such as, for example, 3-Ntyr, NADPH-diaphorase, total nitrite concentration, total nitrate concentration, total nitrite and nitrate concentration, as well as any NO-indicator that has an elevated level in a pancreatic cancer disclosed herein that would be readily envisaged by one skilled in the art, in a subject. In certain embodiments, the subject may have a pancreatic cancer or one or more symptoms thereof. As used herein, the terms “healthy” and “optimal” refer to levels of one or more NO-indicators that would be typical of an equivalent subject that does not have pancreatic cancer.

In certain embodiments, a NOS inhibitor composition, as described herein, may be administered as a suitable dosage form, as described herein, to treat, ameliorate, and/or reduce the severity of a gastrointestinal cancer or one or more symptoms thereof in a subject (“gastrointestinal cancer subject”). As used herein, the term “gastrointestinal cancer” refers to a cancer that occurs and/or originates in an organ of the gastrointestinal tract including, but not limited to the anus, the bile duct, the colon, such as the ascending colon, cecum, descending colon, rectum, sigmoid colon, transverse colon, etc., the esophagus/gastroesophageal junction, the gallbladder, the gastrointestinal stroma, the liver, the renal system, the small intestine, such as the duodenum, ileum, jejunum, etc., and the stomach, such as the antrum, body, cardia, fundus, pylorus, etc.

In some embodiments, a NOS inhibitor composition comprising 7-NI and/or a salt thereof may be administered as a suitable dosage form, as described herein, to treat, ameliorate, and/or reduce the severity of gastrointestinal cancer or one or more symptoms thereof in a gastrointestinal cancer subject. In certain embodiments, an nNOS inhibitor composition comprising 7-NI and/or a salt thereof may be administered to a gastrointestinal cancer subject to treat, ameliorate, and/or reduce the severity of gastrointestinal cancer or one or more symptoms thereof in the gastrointestinal cancer subject: (i) via a route selected from the group consisting of buccally, inhalation, intraarterially, intraarticularly, intracardially, intracerebroventricularly, intradermally, intramedullary, intramuscularly, intraperitoneally, intrathecally, intravenously, intraventricularly, nasally, ophthalmically, orally, rectally, sublabially, sublingually, subcutaneously, topically, and transdermally; and/or (ii) with a frequency selected from the group consisting of four times daily, three times daily, twice daily, once daily, once every two days, once every three days, once every four days, once every five days, once every six days, once a week, once every eight days, once every nine days, once every ten days, once every two weeks, once every three weeks, and once a month; and/or (iii) at a dosage (based on subject weight) selected from the group consisting of 0.1 μg/kg, 0.5 μg/kg, 1 μg/kg, 1.5 μg/kg, 2.0 μg/kg, 2.5 μg/kg, 3.0 μg/kg, 3.5 μg/kg, 4.0 μg/kg, 4.5 μg/kg, 5.0 μg/kg, 10 μg/kg, 15 μg/kg, 20 μg/kg, 25 μg/kg, 30 μg/kg, 35 μg/kg, 40 μg/kg, 45 μg/kg, 50 μg/kg, 55 μg/kg, 60 μg/kg, 65 μg/kg, 70 μg/kg, 75 μg/kg, 80 μg/kg, 85 μg/kg, 90 μg/kg, 95 μg/kg, 100 μg/kg, 150 μg/kg, 200 μg/kg, 250 μg/kg, 300 μg/kg, 350 μg/kg, 400 μg/kg, 450 μg/kg, 500 μg/kg, 550 μg/kg, 600 μg/kg, 650 μg/kg, 700 μg/kg, 750 μg/kg, 800 μg/kg, 850 μg/kg, 900 μg/kg, 1 mg/kg, 1.5 mg/kg, 2.0 mg/kg, 2.5 mg/kg, 3 mg/kg, 3.5 mg/kg, 4.0 mg/kg, 4.5 mg/kg, 5 mg/kg, 5.5 mg/kg, 6 mg/kg, 6.5 mg/kg, 7 mg/kg, 7.5 mg/kg, 8 mg/kg, 8.5 mg/kg, 9 mg/kg, 9.5 mg/kg, 10 mg/kg 10.5 mg/kg, 11 mg/kg, 11.5 mg/kg, 12 mg/kg, 12.5 mg/kg, 13 mg/kg, 13.5 mg/kg, 14 mg/kg, 14.5 mg/kg, 15 mg/kg, 16 mg/kg, 17 mg/kg, 18 mg/kg, 19 mg/kg, 20 mg/kg, 21 mg/kg, 22 mg/kg, 23 mg/kg, 24 mg/kg, 25 mg/kg, 30 mg/kg, 35 mg/kg, 40 mg/kg, 45 mg/kg, 50 mg/kg, 55 mg/kg, 60 mg/kg, 65 mg/kg, 70 mg/kg, 75 mg/kg, 80 mg/kg, 85 mg/kg, 90 mg/kg, 95 mg/kg, 100 mg/kg, 105 mg/kg, 110 mg/kg, 115 mg/kg, 120 mg/kg, 125 mg/kg, 130 mg/kg, 135 mg/kg, 140 mg/kg, 145 mg/kg, 150 mg/kg, 155 mg/kg, 160 mg/kg, 165 mg/kg, 170 mg/kg, 175 mg/kg, 180 mg/kg, 185 mg/kg, 190 mg/kg, 195 mg/kg, 200 mg/kg, and any fraction or integer in between any two of the preceding amounts.

In certain embodiments, a NOS inhibitor composition comprising 3-Br-7-NI and/or a salt thereof may be administered as a suitable dosage form, as described herein, to treat, ameliorate, and/or reduce the severity of gastrointestinal cancer or one or more symptoms thereof in a gastrointestinal cancer subject. In some embodiments, an nNOS inhibitor composition comprising 3-Br-7-NI and/or a salt thereof may be administered to a gastrointestinal cancer subject to treat, ameliorate, and/or reduce the severity of gastrointestinal cancer or one or more symptoms thereof in the gastrointestinal cancer subject: (i) via a route selected from the group consisting of buccally, inhalation, intraarterially, intraarticularly, intracardially, intracerebroventricularly, intradermally, intramedullary, intramuscularly, intraperitoneally, intrathecally, intravenously, intraventricularly, nasally, ophthalmically, orally, rectally, sublabially, sublingually, subcutaneously, topically, and transdermally; and/or (ii) with a frequency selected from the group consisting of four times daily, three times daily, twice daily, once daily, once every two days, once every three days, once every four days, once every five days, once every six days, once a week, once every eight days, once every nine days, once every ten days, once every two weeks, once every three weeks, and once a month; and/or (iii) at a dosage (based on subject weight) selected from the group consisting of 0.1 μg/kg, 0.5 μg/kg, 1 μg/kg, 1.5 μg/kg, 2.0 μg/kg, 2.5 μg/kg, 3.0 μg/kg, 3.5 μg/kg, 4.0 μg/kg, 4.5 μg/kg, 5.0 μg/kg, 10 μg/kg, 15 μg/kg, 20 μg/kg, 25 μg/kg, 30 μg/kg, 35 μg/kg, 40 μg/kg, 45 μg/kg, 50 μg/kg, 55 μg/kg, 60 μg/kg, 65 μg/kg, 70 μg/kg, 75 μg/kg, 80 μg/kg, 85 μg/kg, 90 μg/kg, 95 μg/kg, 100 μg/kg, 150 μg/kg, 200 μg/kg, 250 μg/kg, 300 μg/kg, 350 μg/kg, 400 μg/kg, 450 μg/kg, 500 μg/kg, 550 μg/kg, 600 μg/kg, 650 μg/kg, 700 μg/kg, 750 μg/kg, 800 μg/kg, 850 μg/kg, 900 μg/kg, 1 mg/kg, 1.5 mg/kg, 2.0 mg/kg, 2.5 mg/kg, 3 mg/kg, 3.5 mg/kg, 4.0 mg/kg, 4.5 mg/kg, 5 mg/kg, 5.5 mg/kg, 6 mg/kg, 6.5 mg/kg, 7 mg/kg, 7.5 mg/kg, 8 mg/kg, 8.5 mg/kg, 9 mg/kg, 9.5 mg/kg, 10 mg/kg 10.5 mg/kg, 11 mg/kg, 11.5 mg/kg, 12 mg/kg, 12.5 mg/kg, 13 mg/kg, 13.5 mg/kg, 14 mg/kg, 14.5 mg/kg, 15 mg/kg, 16 mg/kg, 17 mg/kg, 18 mg/kg, 19 mg/kg, 20 mg/kg, 21 mg/kg, 22 mg/kg, 23 mg/kg, 24 mg/kg, 25 mg/kg, 30 mg/kg, 35 mg/kg, 40 mg/kg, 45 mg/kg, 50 mg/kg, 55 mg/kg, 60 mg/kg, 65 mg/kg, 70 mg/kg, 75 mg/kg, 80 mg/kg, 85 mg/kg, 90 mg/kg, 95 mg/kg, 100 mg/kg, 105 mg/kg, 110 mg/kg, 115 mg/kg, 120 mg/kg, 125 mg/kg, 130 mg/kg, 135 mg/kg, 140 mg/kg, 145 mg/kg, 150 mg/kg, 155 mg/kg, 160 mg/kg, 165 mg/kg, 170 mg/kg, 175 mg/kg, 180 mg/kg, 185 mg/kg, 190 mg/kg, 195 mg/kg, 200 mg/kg, and any fraction or integer in between any two of the preceding amounts.

In some embodiments, a NOS inhibitor composition comprising L-NPA and/or a salt thereof, such as, for example, L-NPA hydrochloride, may be administered as a suitable dosage form, as described herein, to treat, ameliorate, and/or reduce the severity of gastrointestinal cancer or one or more symptoms thereof in a gastrointestinal cancer subject. In certain embodiments, an nNOS inhibitor composition comprising L-NPA and/or a salt thereof, such as, for example, L-NPA hydrochloride may be administered to a gastrointestinal cancer subject to treat, ameliorate, and/or reduce the severity of gastrointestinal cancer or one or more symptoms thereof in the gastrointestinal cancer subject: (i) via a route selected from the group consisting of buccally, inhalation, intraarterially, intraarticularly, intracardially, intracerebroventricularly, intradermally, intramedullary, intramuscularly, intraperitoneally, intrathecally, intravenously, intraventricularly, nasally, ophthalmically, orally, rectally, sublabially, sublingually, subcutaneously, topically, and transdermally; and/or (ii) with a frequency selected from the group consisting of four times daily, three times daily, twice daily, once daily, once every two days, once every three days, once every four days, once every five days, once every six days, once a week, once every eight days, once every nine days, once every ten days, once every two weeks, once every three weeks, and once a month; and/or (iii) at a dosage (based on subject weight) selected from the group consisting of 0.1 μg/kg, 0.5 μg/kg, 1 μg/kg, 1.5 μg/kg, 2.0 μg/kg, 2.5 μg/kg, 3.0 μg/kg, 3.5 μg/kg, 4.0 μg/kg, 4.5 μg/kg, 5.0 μg/kg, 10 μg/kg, 15 μg/kg, 20 μg/kg, 25 μg/kg, 30 μg/kg, 35 μg/kg, 40 μg/kg, 45 μg/kg, 50 μg/kg, 55 μg/kg, 60 μg/kg, 65 μg/kg, 70 μg/kg, 75 μg/kg, 80 μg/kg, 85 μg/kg, 90 μg/kg, 95 μg/kg, 100 μg/kg, 150 μg/kg, 200 μg/kg, 250 μg/kg, 300 μg/kg, 350 μg/kg, 400 μg/kg, 450 μg/kg, 500 μg/kg, 550 μg/kg, 600 μg/kg, 650 μg/kg, 700 μg/kg, 750 μg/kg, 800 μg/kg, 850 μg/kg, 900 μg/kg, 1 mg/kg, 1.5 mg/kg, 2.0 mg/kg, 2.5 mg/kg, 3 mg/kg, 3.5 mg/kg, 4.0 mg/kg, 4.5 mg/kg, 5 mg/kg, 5.5 mg/kg, 6 mg/kg, 6.5 mg/kg, 7 mg/kg, 7.5 mg/kg, 8 mg/kg, 8.5 mg/kg, 9 mg/kg, 9.5 mg/kg, 10 mg/kg 10.5 mg/kg, 11 mg/kg, 11.5 mg/kg, 12 mg/kg, 12.5 mg/kg, 13 mg/kg, 13.5 mg/kg, 14 mg/kg, 14.5 mg/kg, 15 mg/kg, 16 mg/kg, 17 mg/kg, 18 mg/kg, 19 mg/kg, 20 mg/kg, 21 mg/kg, 22 mg/kg, 23 mg/kg, 24 mg/kg, 25 mg/kg, 30 mg/kg, 35 mg/kg, 40 mg/kg, 45 mg/kg, 50 mg/kg, 55 mg/kg, 60 mg/kg, 65 mg/kg, 70 mg/kg, 75 mg/kg, 80 mg/kg, 85 mg/kg, 90 mg/kg, 95 mg/kg, 100 mg/kg, 105 mg/kg, 110 mg/kg, 115 mg/kg, 120 mg/kg, 125 mg/kg, 130 mg/kg, 135 mg/kg, 140 mg/kg, 145 mg/kg, 150 mg/kg, 155 mg/kg, 160 mg/kg, 165 mg/kg, 170 mg/kg, 175 mg/kg, 180 mg/kg, 185 mg/kg, 190 mg/kg, 195 mg/kg, 200 mg/kg, and any fraction or integer in between any two of the preceding amounts.

In certain embodiments, a NOS inhibitor composition comprising L-NIO and/or a salt thereof, such as, for example, L-NIO dihydrochloride, may be administered as a suitable dosage form, as described herein, to treat, ameliorate, and/or reduce the severity of gastrointestinal cancer or one or more symptoms thereof in a gastrointestinal cancer subject. In some embodiments, an nNOS inhibitor composition comprising L-NIO and/or a salt thereof, such as, for example, L-NIO dihydrochloride, may be administered to a gastrointestinal cancer subject to treat, ameliorate, and/or reduce the severity of gastrointestinal cancer or one or more symptoms thereof in the gastrointestinal cancer subject: (i) via a route selected from the group consisting of buccally, inhalation, intraarterially, intraarticularly, intracardially, intracerebroventricularly, intradermally, intramedullary, intramuscularly, intraperitoneally, intrathecally, intravenously, intraventricularly, nasally, ophthalmically, orally, rectally, sublabially, sublingually, subcutaneously, topically, and transdermally; and/or (ii) with a frequency selected from the group consisting of four times daily, three times daily, twice daily, once daily, once every two days, once every three days, once every four days, once every five days, once every six days, once a week, once every eight days, once every nine days, once every ten days, once every two weeks, once every three weeks, and once a month; and/or (iii) at a dosage (based on subject weight) selected from the group consisting of 0.1 μg/kg, 0.5 μg/kg, 1 μg/kg, 1.5 μg/kg, 2.0 μg/kg, 2.5 μg/kg, 3.0 μg/kg, 3.5 μg/kg, 4.0 μg/kg, 4.5 μg/kg, 5.0 μg/kg, 10 μg/kg, 15 μg/kg, 20 μg/kg, 25 μg/kg, 30 μg/kg, 35 μg/kg, 40 μg/kg, 45 μg/kg, 50 μg/kg, 55 μg/kg, 60 μg/kg, 65 μg/kg, 70 μg/kg, 75 μg/kg, 80 μg/kg, 85 μg/kg, 90 μg/kg, 95 μg/kg, 100 μg/kg, 150 μg/kg, 200 μg/kg, 250 μg/kg, 300 μg/kg, 350 μg/kg, 400 μg/kg, 450 μg/kg, 500 μg/kg, 550 μg/kg, 600 μg/kg, 650 μg/kg, 700 μg/kg, 750 μg/kg, 800 μg/kg, 850 μg/kg, 900 μg/kg, 1 mg/kg, 1.5 mg/kg, 2.0 mg/kg, 2.5 mg/kg, 3 mg/kg, 3.5 mg/kg, 4.0 mg/kg, 4.5 mg/kg, 5 mg/kg, 5.5 mg/kg, 6 mg/kg, 6.5 mg/kg, 7 mg/kg, 7.5 mg/kg, 8 mg/kg, 8.5 mg/kg, 9 mg/kg, 9.5 mg/kg, 10 mg/kg 10.5 mg/kg, 11 mg/kg, 11.5 mg/kg, 12 mg/kg, 12.5 mg/kg, 13 mg/kg, 13.5 mg/kg, 14 mg/kg, 14.5 mg/kg, 15 mg/kg, 16 mg/kg, 17 mg/kg, 18 mg/kg, 19 mg/kg, 20 mg/kg, 21 mg/kg, 22 mg/kg, 23 mg/kg, 24 mg/kg, 25 mg/kg, 30 mg/kg, 35 mg/kg, 40 mg/kg, 45 mg/kg, 50 mg/kg, 55 mg/kg, 60 mg/kg, 65 mg/kg, 70 mg/kg, 75 mg/kg, 80 mg/kg, 85 mg/kg, 90 mg/kg, 95 mg/kg, 100 mg/kg, 105 mg/kg, 110 mg/kg, 115 mg/kg, 120 mg/kg, 125 mg/kg, 130 mg/kg, 135 mg/kg, 140 mg/kg, 145 mg/kg, 150 mg/kg, 155 mg/kg, 160 mg/kg, 165 mg/kg, 170 mg/kg, 175 mg/kg, 180 mg/kg, 185 mg/kg, 190 mg/kg, 195 mg/kg, 200 mg/kg, and any fraction or integer in between any two of the preceding amounts.

In some embodiments, a NOS inhibitor composition comprising ARL-17477 and/or a salt thereof, such as, for example, ARL-17477 dihydrochloride, may be administered as a suitable dosage form, as described herein, to treat, ameliorate, and/or reduce the severity of gastrointestinal cancer or one or more symptoms thereof in a gastrointestinal cancer subject. In certain embodiments, an nNOS inhibitor composition comprising ARL-17477 and/or a salt thereof, such as, for example, ARL-17477 dihydrochloride, may be administered to a gastrointestinal cancer subject to treat, ameliorate, and/or reduce the severity of gastrointestinal cancer or one or more symptoms thereof in the gastrointestinal cancer subject: (i) via a route selected from the group consisting of buccally, inhalation, intraarterially, intraarticularly, intracardially, intracerebroventricularly, intradermally, intramedullary, intramuscularly, intraperitoneally, intrathecally, intravenously, intraventricularly, nasally, ophthalmically, orally, rectally, sublabially, sublingually, subcutaneously, topically, and transdermally; and/or (ii) with a frequency selected from the group consisting of four times daily, three times daily, twice daily, once daily, once every two days, once every three days, once every four days, once every five days, once every six days, once a week, once every eight days, once every nine days, once every ten days, once every two weeks, once every three weeks, and once a month; and/or (iii) at a dosage (based on subject weight) selected from the group consisting of 0.1 μg/kg, 0.5 μg/kg, 1 μg/kg, 1.5 μg/kg, 2.0 μg/kg, 2.5 μg/kg, 3.0 μg/kg, 3.5 μg/kg, 4.0 μg/kg, 4.5 μg/kg, 5.0 μg/kg, 10 μg/kg, 15 μg/kg, 20 μg/kg, 25 μg/kg, 30 μg/kg, 35 μg/kg, 40 μg/kg, 45 μg/kg, 50 μg/kg, 55 μg/kg, 60 μg/kg, 65 μg/kg, 70 μg/kg, 75 μg/kg, 80 μg/kg, 85 μg/kg, 90 μg/kg, 95 μg/kg, 100 μg/kg, 150 μg/kg, 200 μg/kg, 250 μg/kg, 300 μg/kg, 350 μg/kg, 400 μg/kg, 450 μg/kg, 500 μg/kg, 550 μg/kg, 600 μg/kg, 650 μg/kg, 700 μg/kg, 750 μg/kg, 800 μg/kg, 850 μg/kg, 900 μg/kg, 1 mg/kg, 1.5 mg/kg, 2.0 mg/kg, 2.5 mg/kg, 3 mg/kg, 3.5 mg/kg, 4.0 mg/kg, 4.5 mg/kg, 5 mg/kg, 5.5 mg/kg, 6 mg/kg, 6.5 mg/kg, 7 mg/kg, 7.5 mg/kg, 8 mg/kg, 8.5 mg/kg, 9 mg/kg, 9.5 mg/kg, 10 mg/kg 10.5 mg/kg, 11 mg/kg, 11.5 mg/kg, 12 mg/kg, 12.5 mg/kg, 13 mg/kg, 13.5 mg/kg, 14 mg/kg, 14.5 mg/kg, 15 mg/kg, 16 mg/kg, 17 mg/kg, 18 mg/kg, 19 mg/kg, 20 mg/kg, 21 mg/kg, 22 mg/kg, 23 mg/kg, 24 mg/kg, 25 mg/kg, 30 mg/kg, 35 mg/kg, 40 mg/kg, 45 mg/kg, 50 mg/kg, 55 mg/kg, 60 mg/kg, 65 mg/kg, 70 mg/kg, 75 mg/kg, 80 mg/kg, 85 mg/kg, 90 mg/kg, 95 mg/kg, 100 mg/kg, 105 mg/kg, 110 mg/kg, 115 mg/kg, 120 mg/kg, 125 mg/kg, 130 mg/kg, 135 mg/kg, 140 mg/kg, 145 mg/kg, 150 mg/kg, 155 mg/kg, 160 mg/kg, 165 mg/kg, 170 mg/kg, 175 mg/kg, 180 mg/kg, 185 mg/kg, 190 mg/kg, 195 mg/kg, 200 mg/kg, and any fraction or integer in between any two of the preceding amounts.

In certain embodiments, a NOS inhibitor composition comprising L-NNA and/or a salt thereof may be administered as a suitable dosage form, as described herein, to treat, ameliorate, and/or reduce the severity of gastrointestinal cancer or one or more symptoms thereof in a gastrointestinal cancer subject. In some embodiments, an nNOS inhibitor composition comprising L-NNA and/or a salt thereof may be administered to a gastrointestinal cancer subject to treat, ameliorate, and/or reduce the severity of gastrointestinal cancer or one or more symptoms thereof in the gastrointestinal cancer subject: (i) via a route selected from the group consisting of buccally, inhalation, intraarterially, intraarticularly, intracardially, intracerebroventricularly, intradermally, intramedullary, intramuscularly, intraperitoneally, intrathecally, intravenously, intraventricularly, nasally, ophthalmically, orally, rectally, sublabially, sublingually, subcutaneously, topically, and transdermally; and/or (ii) with a frequency selected from the group consisting of four times daily, three times daily, twice daily, once daily, once every two days, once every three days, once every four days, once every five days, once every six days, once a week, once every eight days, once every nine days, once every ten days, once every two weeks, once every three weeks, and once a month; and/or (iii) at a dosage (based on subject weight) selected from the group consisting of 0.1 μg/kg, 0.5 μg/kg, 1 μg/kg, 1.5 μg/kg, 2.0 μg/kg, 2.5 μg/kg, 3.0 μg/kg, 3.5 μg/kg, 4.0 μg/kg, 4.5 μg/kg, 5.0 μg/kg, 10 μg/kg, 15 μg/kg, 20 μg/kg, 25 μg/kg, 30 μg/kg, 35 μg/kg, 40 μg/kg, 45 μg/kg, 50 μg/kg, 55 μg/kg, 60 μg/kg, 65 μg/kg, 70 μg/kg, 75 μg/kg, 80 μg/kg, 85 μg/kg, 90 μg/kg, 95 μg/kg, 100 μg/kg, 150 μg/kg, 200 μg/kg, 250 μg/kg, 300 μg/kg, 350 μg/kg, 400 μg/kg, 450 μg/kg, 500 μg/kg, 550 μg/kg, 600 μg/kg, 650 μg/kg, 700 μg/kg, 750 μg/kg, 800 μg/kg, 850 μg/kg, 900 μg/kg, 1 mg/kg, 1.5 mg/kg, 2.0 mg/kg, 2.5 mg/kg, 3 mg/kg, 3.5 mg/kg, 4.0 mg/kg, 4.5 mg/kg, 5 mg/kg, 5.5 mg/kg, 6 mg/kg, 6.5 mg/kg, 7 mg/kg, 7.5 mg/kg, 8 mg/kg, 8.5 mg/kg, 9 mg/kg, 9.5 mg/kg, 10 mg/kg 10.5 mg/kg, 11 mg/kg, 11.5 mg/kg, 12 mg/kg, 12.5 mg/kg, 13 mg/kg, 13.5 mg/kg, 14 mg/kg, 14.5 mg/kg, 15 mg/kg, 16 mg/kg, 17 mg/kg, 18 mg/kg, 19 mg/kg, 20 mg/kg, 21 mg/kg, 22 mg/kg, 23 mg/kg, 24 mg/kg, 25 mg/kg, 30 mg/kg, 35 mg/kg, 40 mg/kg, 45 mg/kg, 50 mg/kg, 55 mg/kg, 60 mg/kg, 65 mg/kg, 70 mg/kg, 75 mg/kg, 80 mg/kg, 85 mg/kg, 90 mg/kg, 95 mg/kg, 100 mg/kg, 105 mg/kg, 110 mg/kg, 115 mg/kg, 120 mg/kg, 125 mg/kg, 130 mg/kg, 135 mg/kg, 140 mg/kg, 145 mg/kg, 150 mg/kg, 155 mg/kg, 160 mg/kg, 165 mg/kg, 170 mg/kg, 175 mg/kg, 180 mg/kg, 185 mg/kg, 190 mg/kg, 195 mg/kg, 200 mg/kg, and any fraction or integer in between any two of the preceding amounts.

In some embodiments, a NOS inhibitor composition comprising L-NAME and/or a salt thereof, such as, for example, L-NAME hydrochloride, may be administered as a suitable dosage form, as described herein, to treat, ameliorate, and/or reduce the severity of gastrointestinal cancer or one or more symptoms thereof in a gastrointestinal cancer subject. In certain embodiments, an nNOS inhibitor composition comprising L-NAME and/or a salt thereof, such as, for example, L-NAME hydrochloride, may be administered to a gastrointestinal cancer subject to treat, ameliorate, and/or reduce the severity of gastrointestinal cancer or one or more symptoms thereof in the gastrointestinal cancer subject: (i) via a route selected from the group consisting of buccally, inhalation, intraarterially, intraarticularly, intracardially, intracerebroventricularly, intradermally, intramedullary, intramuscularly, intraperitoneally, intrathecally, intravenously, intraventricularly, nasally, ophthalmically, orally, rectally, sublabially, sublingually, subcutaneously, topically, and transdermally; and/or (ii) with a frequency selected from the group consisting of four times daily, three times daily, twice daily, once daily, once every two days, once every three days, once every four days, once every five days, once every six days, once a week, once every eight days, once every nine days, once every ten days, once every two weeks, once every three weeks, and once a month; and/or (iii) at a dosage (based on subject weight) selected from the group consisting of 0.1 μg/kg, 0.5 μg/kg, 1 μg/kg, 1.5 μg/kg, 2.0 μg/kg, 2.5 μg/kg, 3.0 μg/kg, 3.5 μg/kg, 4.0 μg/kg, 4.5 μg/kg, 5.0 μg/kg, 10 μg/kg, 15 μg/kg, 20 μg/kg, 25 μg/kg, 30 μg/kg, 35 μg/kg, 40 μg/kg, 45 μg/kg, 50 μg/kg, 55 μg/kg, 60 μg/kg, 65 μg/kg, 70 μg/kg, 75 μg/kg, 80 μg/kg, 85 μg/kg, 90 μg/kg, 95 μg/kg, 100 μg/kg, 150 μg/kg, 200 μg/kg, 250 μg/kg, 300 μg/kg, 350 μg/kg, 400 μg/kg, 450 μg/kg, 500 μg/kg, 550 μg/kg, 600 μg/kg, 650 μg/kg, 700 μg/kg, 750 μg/kg, 800 μg/kg, 850 μg/kg, 900 μg/kg, 1 mg/kg, 1.5 mg/kg, 2.0 mg/kg, 2.5 mg/kg, 3 mg/kg, 3.5 mg/kg, 4.0 mg/kg, 4.5 mg/kg, 5 mg/kg, 5.5 mg/kg, 6 mg/kg, 6.5 mg/kg, 7 mg/kg, 7.5 mg/kg, 8 mg/kg, 8.5 mg/kg, 9 mg/kg, 9.5 mg/kg, 10 mg/kg 10.5 mg/kg, 11 mg/kg, 11.5 mg/kg, 12 mg/kg, 12.5 mg/kg, 13 mg/kg, 13.5 mg/kg, 14 mg/kg, 14.5 mg/kg, 15 mg/kg, 16 mg/kg, 17 mg/kg, 18 mg/kg, 19 mg/kg, 20 mg/kg, 21 mg/kg, 22 mg/kg, 23 mg/kg, 24 mg/kg, 25 mg/kg, 30 mg/kg, 35 mg/kg, 40 mg/kg, 45 mg/kg, 50 mg/kg, 55 mg/kg, 60 mg/kg, 65 mg/kg, 70 mg/kg, 75 mg/kg, 80 mg/kg, 85 mg/kg, 90 mg/kg, 95 mg/kg, 100 mg/kg, 105 mg/kg, 110 mg/kg, 115 mg/kg, 120 mg/kg, 125 mg/kg, 130 mg/kg, 135 mg/kg, 140 mg/kg, 145 mg/kg, 150 mg/kg, 155 mg/kg, 160 mg/kg, 165 mg/kg, 170 mg/kg, 175 mg/kg, 180 mg/kg, 185 mg/kg, 190 mg/kg, 195 mg/kg, 200 mg/kg, and any fraction or integer in between any two of the preceding amounts.

In certain embodiments, a NOS inhibitor composition comprising L-NMMA and/or a salt thereof, such as, for example, L-NMMA acetate, may be administered as a suitable dosage form, as described herein, to treat, ameliorate, and/or reduce the severity of gastrointestinal cancer or one or more symptoms thereof in a gastrointestinal cancer subject. In some embodiments, an nNOS inhibitor composition comprising L-NMMA and/or a salt thereof, such as, for example, L-NMMA acetate, may be administered to a gastrointestinal cancer subject to treat, ameliorate, and/or reduce the severity of gastrointestinal cancer or one or more symptoms thereof in the gastrointestinal cancer subject: (i) via a route selected from the group consisting of buccally, inhalation, intraarterially, intraarticularly, intracardially, intracerebroventricularly, intradermally, intramedullary, intramuscularly, intraperitoneally, intrathecally, intravenously, intraventricularly, nasally, ophthalmically, orally, rectally, sublabially, sublingually, subcutaneously, topically, and transdermally; and/or (ii) with a frequency selected from the group consisting of four times daily, three times daily, twice daily, once daily, once every two days, once every three days, once every four days, once every five days, once every six days, once a week, once every eight days, once every nine days, once every ten days, once every two weeks, once every three weeks, and once a month; and/or (iii) at a dosage (based on subject weight) selected from the group consisting of 0.1 μg/kg, 0.5 μg/kg, 1 μg/kg, 1.5 μg/kg, 2.0 μg/kg, 2.5 μg/kg, 3.0 μg/kg, 3.5 μg/kg, 4.0 μg/kg, 4.5 μg/kg, 5.0 μg/kg, 10 μg/kg, 15 μg/kg, 20 μg/kg, 25 μg/kg, 30 μg/kg, 35 μg/kg, 40 μg/kg, 45 μg/kg, 50 μg/kg, 55 μg/kg, 60 μg/kg, 65 μg/kg, 70 μg/kg, 75 μg/kg, 80 μg/kg, 85 μg/kg, 90 μg/kg, 95 μg/kg, 100 μg/kg, 150 μg/kg, 200 μg/kg, 250 μg/kg, 300 μg/kg, 350 μg/kg, 400 μg/kg, 450 μg/kg, 500 μg/kg, 550 μg/kg, 600 μg/kg, 650 μg/kg, 700 μg/kg, 750 μg/kg, 800 μg/kg, 850 μg/kg, 900 μg/kg, 1 mg/kg, 1.5 mg/kg, 2.0 mg/kg, 2.5 mg/kg, 3 mg/kg, 3.5 mg/kg, 4.0 mg/kg, 4.5 mg/kg, 5 mg/kg, 5.5 mg/kg, 6 mg/kg, 6.5 mg/kg, 7 mg/kg, 7.5 mg/kg, 8 mg/kg, 8.5 mg/kg, 9 mg/kg, 9.5 mg/kg, 10 mg/kg 10.5 mg/kg, 11 mg/kg, 11.5 mg/kg, 12 mg/kg, 12.5 mg/kg, 13 mg/kg, 13.5 mg/kg, 14 mg/kg, 14.5 mg/kg, 15 mg/kg, 16 mg/kg, 17 mg/kg, 18 mg/kg, 19 mg/kg, 20 mg/kg, 21 mg/kg, 22 mg/kg, 23 mg/kg, 24 mg/kg, 25 mg/kg, 30 mg/kg, 35 mg/kg, 40 mg/kg, 45 mg/kg, 50 mg/kg, 55 mg/kg, 60 mg/kg, 65 mg/kg, 70 mg/kg, 75 mg/kg, 80 mg/kg, 85 mg/kg, 90 mg/kg, 95 mg/kg, 100 mg/kg, 105 mg/kg, 110 mg/kg, 115 mg/kg, 120 mg/kg, 125 mg/kg, 130 mg/kg, 135 mg/kg, 140 mg/kg, 145 mg/kg, 150 mg/kg, 155 mg/kg, 160 mg/kg, 165 mg/kg, 170 mg/kg, 175 mg/kg, 180 mg/kg, 185 mg/kg, 190 mg/kg, 195 mg/kg, 200 mg/kg, and any fraction or integer in between any two of the preceding amounts.

In some embodiments, a NOS inhibitor composition comprising GW-274150 and/or a salt thereof, such as, for example, GW-274150 phosphate, may be administered as a suitable dosage form, as described herein, to treat, ameliorate, and/or reduce the severity of gastrointestinal cancer or one or more symptoms thereof in a gastrointestinal cancer subject. In certain embodiments, an nNOS inhibitor composition comprising GW-274150 and/or a salt thereof, such as, for example, GW-274150 phosphate, may be administered to a gastrointestinal cancer subject to treat, ameliorate, and/or reduce the severity of gastrointestinal cancer or one or more symptoms thereof in the gastrointestinal cancer subject: (i) via a route selected from the group consisting of buccally, inhalation, intraarterially, intraarticularly, intracardially, intracerebroventricularly, intradermally, intramedullary, intramuscularly, intraperitoneally, intrathecally, intravenously, intraventricularly, nasally, ophthalmically, orally, rectally, sublabially, sublingually, subcutaneously, topically, and transdermally; and/or (ii) with a frequency selected from the group consisting of four times daily, three times daily, twice daily, once daily, once every two days, once every three days, once every four days, once every five days, once every six days, once a week, once every eight days, once every nine days, once every ten days, once every two weeks, once every three weeks, and once a month; and/or (iii) at a dosage (based on subject weight) selected from the group consisting of 0.1 μg/kg, 0.5 μg/kg, 1 μg/kg, 1.5 μg/kg, 2.0 μg/kg, 2.5 μg/kg, 3.0 μg/kg, 3.5 μg/kg, 4.0 μg/kg, 4.5 μg/kg, 5.0 μg/kg, 10 μg/kg, 15 μg/kg, 20 μg/kg, 25 μg/kg, 30 μg/kg, 35 μg/kg, 40 μg/kg, 45 μg/kg, 50 μg/kg, 55 μg/kg, 60 μg/kg, 65 μg/kg, 70 μg/kg, 75 μg/kg, 80 μg/kg, 85 μg/kg, 90 μg/kg, 95 μg/kg, 100 μg/kg, 150 μg/kg, 200 μg/kg, 250 μg/kg, 300 μg/kg, 350 μg/kg, 400 μg/kg, 450 μg/kg, 500 μg/kg, 550 μg/kg, 600 μg/kg, 650 μg/kg, 700 μg/kg, 750 μg/kg, 800 μg/kg, 850 μg/kg, 900 μg/kg, 1 mg/kg, 1.5 mg/kg, 2.0 mg/kg, 2.5 mg/kg, 3 mg/kg, 3.5 mg/kg, 4.0 mg/kg, 4.5 mg/kg, 5 mg/kg, 5.5 mg/kg, 6 mg/kg, 6.5 mg/kg, 7 mg/kg, 7.5 mg/kg, 8 mg/kg, 8.5 mg/kg, 9 mg/kg, 9.5 mg/kg, 10 mg/kg 10.5 mg/kg, 11 mg/kg, 11.5 mg/kg, 12 mg/kg, 12.5 mg/kg, 13 mg/kg, 13.5 mg/kg, 14 mg/kg, 14.5 mg/kg, 15 mg/kg, 16 mg/kg, 17 mg/kg, 18 mg/kg, 19 mg/kg, 20 mg/kg, 21 mg/kg, 22 mg/kg, 23 mg/kg, 24 mg/kg, 25 mg/kg, 30 mg/kg, 35 mg/kg, 40 mg/kg, 45 mg/kg, 50 mg/kg, 55 mg/kg, 60 mg/kg, 65 mg/kg, 70 mg/kg, 75 mg/kg, 80 mg/kg, 85 mg/kg, 90 mg/kg, 95 mg/kg, 100 mg/kg, 105 mg/kg, 110 mg/kg, 115 mg/kg, 120 mg/kg, 125 mg/kg, 130 mg/kg, 135 mg/kg, 140 mg/kg, 145 mg/kg, 150 mg/kg, 155 mg/kg, 160 mg/kg, 165 mg/kg, 170 mg/kg, 175 mg/kg, 180 mg/kg, 185 mg/kg, 190 mg/kg, 195 mg/kg, 200 mg/kg, and any fraction or integer in between any two of the preceding amounts.

In certain embodiments, a NOS inhibitor composition comprising L-NIL and/or a salt thereof, such as, for example, L-NIL hydrochloride, may be administered as a suitable dosage form, as described herein, to treat, ameliorate, and/or reduce the severity of gastrointestinal cancer or one or more symptoms thereof in a gastrointestinal cancer subject. In some embodiments, an nNOS inhibitor composition comprising L-NIL and/or a salt thereof, such as, for example, L-NIL hydrochloride, may be administered to a gastrointestinal cancer subject to treat, ameliorate, and/or reduce the severity of gastrointestinal cancer or one or more symptoms thereof in the gastrointestinal cancer subject: (i) via a route selected from the group consisting of buccally, inhalation, intraarterially, intraarticularly, intracardially, intracerebroventricularly, intradermally, intramedullary, intramuscularly, intraperitoneally, intrathecally, intravenously, intraventricularly, nasally, ophthalmically, orally, rectally, sublabially, sublingually, subcutaneously, topically, and transdermally; and/or (ii) with a frequency selected from the group consisting of four times daily, three times daily, twice daily, once daily, once every two days, once every three days, once every four days, once every five days, once every six days, once a week, once every eight days, once every nine days, once every ten days, once every two weeks, once every three weeks, and once a month; and/or (iii) at a dosage (based on subject weight) selected from the group consisting of 0.1 μg/kg, 0.5 μg/kg, 1 μg/kg, 1.5 μg/kg, 2.0 μg/kg, 2.5 μg/kg, 3.0 μg/kg, 3.5 μg/kg, 4.0 μg/kg, 4.5 μg/kg, 5.0 μg/kg, 10 μg/kg, 15 μg/kg, 20 μg/kg, 25 μg/kg, 30 μg/kg, 35 μg/kg, 40 μg/kg, 45 μg/kg, 50 μg/kg, 55 μg/kg, 60 μg/kg, 65 μg/kg, 70 μg/kg, 75 μg/kg, 80 μg/kg, 85 μg/kg, 90 μg/kg, 95 μg/kg, 100 μg/kg, 150 μg/kg, 200 μg/kg, 250 μg/kg, 300 μg/kg, 350 μg/kg, 400 μg/kg, 450 μg/kg, 500 μg/kg, 550 μg/kg, 600 μg/kg, 650 μg/kg, 700 μg/kg, 750 μg/kg, 800 μg/kg, 850 μg/kg, 900 μg/kg, 1 mg/kg, 1.5 mg/kg, 2.0 mg/kg, 2.5 mg/kg, 3 mg/kg, 3.5 mg/kg, 4.0 mg/kg, 4.5 mg/kg, 5 mg/kg, 5.5 mg/kg, 6 mg/kg, 6.5 mg/kg, 7 mg/kg, 7.5 mg/kg, 8 mg/kg, 8.5 mg/kg, 9 mg/kg, 9.5 mg/kg, 10 mg/kg 10.5 mg/kg, 11 mg/kg, 11.5 mg/kg, 12 mg/kg, 12.5 mg/kg, 13 mg/kg, 13.5 mg/kg, 14 mg/kg, 14.5 mg/kg, 15 mg/kg, 16 mg/kg, 17 mg/kg, 18 mg/kg, 19 mg/kg, 20 mg/kg, 21 mg/kg, 22 mg/kg, 23 mg/kg, 24 mg/kg, 25 mg/kg, 30 mg/kg, 35 mg/kg, 40 mg/kg, 45 mg/kg, 50 mg/kg, 55 mg/kg, 60 mg/kg, 65 mg/kg, 70 mg/kg, 75 mg/kg, 80 mg/kg, 85 mg/kg, 90 mg/kg, 95 mg/kg, 100 mg/kg, 105 mg/kg, 110 mg/kg, 115 mg/kg, 120 mg/kg, 125 mg/kg, 130 mg/kg, 135 mg/kg, 140 mg/kg, 145 mg/kg, 150 mg/kg, 155 mg/kg, 160 mg/kg, 165 mg/kg, 170 mg/kg, 175 mg/kg, 180 mg/kg, 185 mg/kg, 190 mg/kg, 195 mg/kg, 200 mg/kg, and any fraction or integer in between any two of the preceding amounts.

In some embodiments, a NOS inhibitor composition comprising 1400W and/or a salt thereof, such as, for example, 1400W dihydrochloride, may be administered as a suitable dosage form, as described herein, to treat, ameliorate, and/or reduce the severity of gastrointestinal cancer or one or more symptoms thereof in a gastrointestinal cancer subject. In certain embodiments, an nNOS inhibitor composition comprising 1400W and/or a salt thereof, such as, for example, 1400W dihydrochloride, may be administered to a gastrointestinal cancer subject to treat, ameliorate, and/or reduce the severity of gastrointestinal cancer or one or more symptoms thereof in the gastrointestinal cancer subject: (i) via a route selected from the group consisting of buccally, inhalation, intraarterially, intraarticularly, intracardially, intracerebroventricularly, intradermally, intramedullary, intramuscularly, intraperitoneally, intrathecally, intravenously, intraventricularly, nasally, ophthalmically, orally, rectally, sublabially, sublingually, subcutaneously, topically, and transdermally; and/or (ii) with a frequency selected from the group consisting of four times daily, three times daily, twice daily, once daily, once every two days, once every three days, once every four days, once every five days, once every six days, once a week, once every eight days, once every nine days, once every ten days, once every two weeks, once every three weeks, and once a month; and/or (iii) at a dosage (based on subject weight) selected from the group consisting of 0.1 μg/kg, 0.5 μg/kg, 1 μg/kg, 1.5 μg/kg, 2.0 μg/kg, 2.5 μg/kg, 3.0 μg/kg, 3.5 μg/kg, 4.0 μg/kg, 4.5 μg/kg, 5.0 μg/kg, 10 μg/kg, 15 μg/kg, 20 μg/kg, 25 μg/kg, 30 μg/kg, 35 μg/kg, 40 μg/kg, 45 μg/kg, 50 μg/kg, 55 μg/kg, 60 μg/kg, 65 μg/kg, 70 μg/kg, 75 μg/kg, 80 μg/kg, 85 μg/kg, 90 μg/kg, 95 μg/kg, 100 μg/kg, 150 μg/kg, 200 μg/kg, 250 μg/kg, 300 μg/kg, 350 μg/kg, 400 μg/kg, 450 μg/kg, 500 μg/kg, 550 μg/kg, 600 μg/kg, 650 μg/kg, 700 μg/kg, 750 μg/kg, 800 μg/kg, 850 μg/kg, 900 μg/kg, 1 mg/kg, 1.5 mg/kg, 2.0 mg/kg, 2.5 mg/kg, 3 mg/kg, 3.5 mg/kg, 4.0 mg/kg, 4.5 mg/kg, 5 mg/kg, 5.5 mg/kg, 6 mg/kg, 6.5 mg/kg, 7 mg/kg, 7.5 mg/kg, 8 mg/kg, 8.5 mg/kg, 9 mg/kg, 9.5 mg/kg, 10 mg/kg 10.5 mg/kg, 11 mg/kg, 11.5 mg/kg, 12 mg/kg, 12.5 mg/kg, 13 mg/kg, 13.5 mg/kg, 14 mg/kg, 14.5 mg/kg, 15 mg/kg, 16 mg/kg, 17 mg/kg, 18 mg/kg, 19 mg/kg, 20 mg/kg, 21 mg/kg, 22 mg/kg, 23 mg/kg, 24 mg/kg, 25 mg/kg, 30 mg/kg, 35 mg/kg, 40 mg/kg, 45 mg/kg, 50 mg/kg, 55 mg/kg, 60 mg/kg, 65 mg/kg, 70 mg/kg, 75 mg/kg, 80 mg/kg, 85 mg/kg, 90 mg/kg, 95 mg/kg, 100 mg/kg, 105 mg/kg, 110 mg/kg, 115 mg/kg, 120 mg/kg, 125 mg/kg, 130 mg/kg, 135 mg/kg, 140 mg/kg, 145 mg/kg, 150 mg/kg, 155 mg/kg, 160 mg/kg, 165 mg/kg, 170 mg/kg, 175 mg/kg, 180 mg/kg, 185 mg/kg, 190 mg/kg, 195 mg/kg, 200 mg/kg, and any fraction or integer in between any two of the preceding amounts.

In certain embodiments, a NOS inhibitor composition comprising OKN-007 and/or a salt thereof, such as, for example, OKN-007 sodium, may be administered as a suitable dosage form, as described herein, to treat, ameliorate, and/or reduce the severity of gastrointestinal cancer or one or more symptoms thereof in a gastrointestinal cancer subject. In some embodiments, an nNOS inhibitor composition comprising OKN-007 and/or a salt thereof, such as, for example, OKN-007 sodium, may be administered to a gastrointestinal cancer subject to treat, ameliorate, and/or reduce the severity of gastrointestinal cancer or one or more symptoms thereof in the gastrointestinal cancer subject: (i) via a route selected from the group consisting of buccally, inhalation, intraarterially, intraarticularly, intracardially, intracerebroventricularly, intradermally, intramedullary, intramuscularly, intraperitoneally, intrathecally, intravenously, intraventricularly, nasally, ophthalmically, orally, rectally, sublabially, sublingually, subcutaneously, topically, and transdermally; and/or (ii) with a frequency selected from the group consisting of four times daily, three times daily, twice daily, once daily, once every two days, once every three days, once every four days, once every five days, once every six days, once a week, once every eight days, once every nine days, once every ten days, once every two weeks, once every three weeks, and once a month; and/or (iii) at a dosage (based on subject weight) selected from the group consisting of 0.1 μg/kg, 0.5 μg/kg, 1 μg/kg, 1.5 μg/kg, 2.0 μg/kg, 2.5 μg/kg, 3.0 μg/kg, 3.5 μg/kg, 4.0 μg/kg, 4.5 μg/kg, 5.0 μg/kg, 10 μg/kg, 15 μg/kg, 20 μg/kg, 25 μg/kg, 30 μg/kg, 35 μg/kg, 40 μg/kg, 45 μg/kg, 50 μg/kg, 55 μg/kg, 60 μg/kg, 65 μg/kg, 70 μg/kg, 75 μg/kg, 80 μg/kg, 85 μg/kg, 90 μg/kg, 95 μg/kg, 100 μg/kg, 150 μg/kg, 200 μg/kg, 250 μg/kg, 300 μg/kg, 350 μg/kg, 400 μg/kg, 450 μg/kg, 500 μg/kg, 550 μg/kg, 600 μg/kg, 650 μg/kg, 700 μg/kg, 750 μg/kg, 800 μg/kg, 850 μg/kg, 900 μg/kg, 1 mg/kg, 1.5 mg/kg, 2.0 mg/kg, 2.5 mg/kg, 3 mg/kg, 3.5 mg/kg, 4.0 mg/kg, 4.5 mg/kg, 5 mg/kg, 5.5 mg/kg, 6 mg/kg, 6.5 mg/kg, 7 mg/kg, 7.5 mg/kg, 8 mg/kg, 8.5 mg/kg, 9 mg/kg, 9.5 mg/kg, 10 mg/kg 10.5 mg/kg, 11 mg/kg, 11.5 mg/kg, 12 mg/kg, 12.5 mg/kg, 13 mg/kg, 13.5 mg/kg, 14 mg/kg, 14.5 mg/kg, 15 mg/kg, 16 mg/kg, 17 mg/kg, 18 mg/kg, 19 mg/kg, 20 mg/kg, 21 mg/kg, 22 mg/kg, 23 mg/kg, 24 mg/kg, 25 mg/kg, 30 mg/kg, 35 mg/kg, 40 mg/kg, 45 mg/kg, 50 mg/kg, 55 mg/kg, 60 mg/kg, 65 mg/kg, 70 mg/kg, 75 mg/kg, 80 mg/kg, 85 mg/kg, 90 mg/kg, 95 mg/kg, 100 mg/kg, 105 mg/kg, 110 mg/kg, 115 mg/kg, 120 mg/kg, 125 mg/kg, 130 mg/kg, 135 mg/kg, 140 mg/kg, 145 mg/kg, 150 mg/kg, 155 mg/kg, 160 mg/kg, 165 mg/kg, 170 mg/kg, 175 mg/kg, 180 mg/kg, 185 mg/kg, 190 mg/kg, 195 mg/kg, 200 mg/kg, and any fraction or integer in between any two of the preceding amounts.

In some embodiments, a NOS inhibitor composition comprising BYK-191023 and/or a salt thereof, such as, for example, BYK-191023 dihydrochloride, may be administered as a suitable dosage form, as described herein, to treat, ameliorate, and/or reduce the severity of gastrointestinal cancer or one or more symptoms thereof in a gastrointestinal cancer subject. In certain embodiments, an nNOS inhibitor composition comprising BYK-191023 and/or a salt thereof, such as, for example, BYK-191023 dihydrochloride, may be administered to a gastrointestinal cancer subject to treat, ameliorate, and/or reduce the severity of gastrointestinal cancer or one or more symptoms thereof in the gastrointestinal cancer subject: (i) via a route selected from the group consisting of buccally, inhalation, intraarterially, intraarticularly, intracardially, intracerebroventricularly, intradermally, intramedullary, intramuscularly, intraperitoneally, intrathecally, intravenously, intraventricularly, nasally, ophthalmically, orally, rectally, sublabially, sublingually, subcutaneously, topically, and transdermally; and/or (ii) with a frequency selected from the group consisting of four times daily, three times daily, twice daily, once daily, once every two days, once every three days, once every four days, once every five days, once every six days, once a week, once every eight days, once every nine days, once every ten days, once every two weeks, once every three weeks, and once a month; and/or (iii) at a dosage (based on subject weight) selected from the group consisting of 0.1 μg/kg, 0.5 μg/kg, 1 μg/kg, 1.5 μg/kg, 2.0 μg/kg, 2.5 μg/kg, 3.0 μg/kg, 3.5 μg/kg, 4.0 μg/kg, 4.5 μg/kg, 5.0 μg/kg, 10 μg/kg, 15 μg/kg, 20 μg/kg, 25 μg/kg, 30 μg/kg, 35 μg/kg, 40 μg/kg, 45 μg/kg, 50 μg/kg, 55 μg/kg, 60 μg/kg, 65 μg/kg, 70 μg/kg, 75 μg/kg, 80 μg/kg, 85 μg/kg, 90 μg/kg, 95 μg/kg, 100 μg/kg, 150 μg/kg, 200 μg/kg, 250 μg/kg, 300 μg/kg, 350 μg/kg, 400 μg/kg, 450 μg/kg, 500 μg/kg, 550 μg/kg, 600 μg/kg, 650 μg/kg, 700 μg/kg, 750 μg/kg, 800 μg/kg, 850 μg/kg, 900 μg/kg, 1 mg/kg, 1.5 mg/kg, 2.0 mg/kg, 2.5 mg/kg, 3 mg/kg, 3.5 mg/kg, 4.0 mg/kg, 4.5 mg/kg, 5 mg/kg, 5.5 mg/kg, 6 mg/kg, 6.5 mg/kg, 7 mg/kg, 7.5 mg/kg, 8 mg/kg, 8.5 mg/kg, 9 mg/kg, 9.5 mg/kg, 10 mg/kg 10.5 mg/kg, 11 mg/kg, 11.5 mg/kg, 12 mg/kg, 12.5 mg/kg, 13 mg/kg, 13.5 mg/kg, 14 mg/kg, 14.5 mg/kg, 15 mg/kg, 16 mg/kg, 17 mg/kg, 18 mg/kg, 19 mg/kg, 20 mg/kg, 21 mg/kg, 22 mg/kg, 23 mg/kg, 24 mg/kg, 25 mg/kg, 30 mg/kg, 35 mg/kg, 40 mg/kg, 45 mg/kg, 50 mg/kg, 55 mg/kg, 60 mg/kg, 65 mg/kg, 70 mg/kg, 75 mg/kg, 80 mg/kg, 85 mg/kg, 90 mg/kg, 95 mg/kg, 100 mg/kg, 105 mg/kg, 110 mg/kg, 115 mg/kg, 120 mg/kg, 125 mg/kg, 130 mg/kg, 135 mg/kg, 140 mg/kg, 145 mg/kg, 150 mg/kg, 155 mg/kg, 160 mg/kg, 165 mg/kg, 170 mg/kg, 175 mg/kg, 180 mg/kg, 185 mg/kg, 190 mg/kg, 195 mg/kg, 200 mg/kg, and any fraction or integer in between any two of the preceding amounts.

In certain embodiments, a NOS inhibitor composition comprising CM544 and/or a salt thereof, such as, for example, CM544 dihydrochloride, may be administered as a suitable dosage form, as described herein, to treat, ameliorate, and/or reduce the severity of gastrointestinal cancer or one or more symptoms thereof in a gastrointestinal cancer subject. In some embodiments, an nNOS inhibitor composition comprising CM544 and/or a salt thereof, such as, for example, CM544 dihydrochloride, may be administered to a gastrointestinal cancer subject to treat, ameliorate, and/or reduce the severity of gastrointestinal cancer or one or more symptoms thereof in the gastrointestinal cancer subject: (i) via a route selected from the group consisting of buccally, inhalation, intraarterially, intraarticularly, intracardially, intracerebroventricularly, intradermally, intramedullary, intramuscularly, intraperitoneally, intrathecally, intravenously, intraventricularly, nasally, ophthalmically, orally, rectally, sublabially, sublingually, subcutaneously, topically, and transdermally; and/or (ii) with a frequency selected from the group consisting of four times daily, three times daily, twice daily, once daily, once every two days, once every three days, once every four days, once every five days, once every six days, once a week, once every eight days, once every nine days, once every ten days, once every two weeks, once every three weeks, and once a month; and/or (iii) at a dosage (based on subject weight) selected from the group consisting of 0.1 μg/kg, 0.5 μg/kg, 1 μg/kg, 1.5 μg/kg, 2.0 μg/kg, 2.5 μg/kg, 3.0 μg/kg, 3.5 μg/kg, 4.0 μg/kg, 4.5 μg/kg, 5.0 μg/kg, 10 μg/kg, 15 μg/kg, 20 μg/kg, 25 μg/kg, 30 μg/kg, 35 μg/kg, 40 μg/kg, 45 μg/kg, 50 μg/kg, 55 μg/kg, 60 μg/kg, 65 μg/kg, 70 μg/kg, 75 μg/kg, 80 μg/kg, 85 μg/kg, 90 μg/kg, 95 μg/kg, 100 μg/kg, 150 μg/kg, 200 μg/kg, 250 μg/kg, 300 μg/kg, 350 μg/kg, 400 μg/kg, 450 μg/kg, 500 μg/kg, 550 μg/kg, 600 μg/kg, 650 μg/kg, 700 μg/kg, 750 μg/kg, 800 μg/kg, 850 μg/kg, 900 μg/kg, 1 mg/kg, 1.5 mg/kg, 2.0 mg/kg, 2.5 mg/kg, 3 mg/kg, 3.5 mg/kg, 4.0 mg/kg, 4.5 mg/kg, 5 mg/kg, 5.5 mg/kg, 6 mg/kg, 6.5 mg/kg, 7 mg/kg, 7.5 mg/kg, 8 mg/kg, 8.5 mg/kg, 9 mg/kg, 9.5 mg/kg, 10 mg/kg 10.5 mg/kg, 11 mg/kg, 11.5 mg/kg, 12 mg/kg, 12.5 mg/kg, 13 mg/kg, 13.5 mg/kg, 14 mg/kg, 14.5 mg/kg, 15 mg/kg, 16 mg/kg, 17 mg/kg, 18 mg/kg, 19 mg/kg, 20 mg/kg, 21 mg/kg, 22 mg/kg, 23 mg/kg, 24 mg/kg, 25 mg/kg, 30 mg/kg, 35 mg/kg, 40 mg/kg, 45 mg/kg, 50 mg/kg, 55 mg/kg, 60 mg/kg, 65 mg/kg, 70 mg/kg, 75 mg/kg, 80 mg/kg, 85 mg/kg, 90 mg/kg, 95 mg/kg, 100 mg/kg, 105 mg/kg, 110 mg/kg, 115 mg/kg, 120 mg/kg, 125 mg/kg, 130 mg/kg, 135 mg/kg, 140 mg/kg, 145 mg/kg, 150 mg/kg, 155 mg/kg, 160 mg/kg, 165 mg/kg, 170 mg/kg, 175 mg/kg, 180 mg/kg, 185 mg/kg, 190 mg/kg, 195 mg/kg, 200 mg/kg, and any fraction or integer in between any two of the preceding amounts.

In certain embodiments, an nNOS inhibitor composition comprising one or more dsRNAs each comprising at least 30 base pairs, may be administered as a suitable dosage form, as described herein, to treat, ameliorate, and/or reduce the severity of gastrointestinal cancer or one or more symptoms thereof in a gastrointestinal cancer subject. In some embodiments, an nNOS inhibitor composition comprising one or more dsRNAs, each comprising at least 30 base pairs, may be administered to a gastrointestinal cancer subject to treat, ameliorate, and/or reduce the severity of gastrointestinal cancer or one or more symptoms thereof in the gastrointestinal cancer subject: (i) via a route selected from the group consisting of buccally, inhalation, intraarterially, intraarticularly, intracardially, intracerebroventricularly, intradermally, intramedullary, intramuscularly, intraperitoneally, intrathecally, intravenously, intraventricularly, nasally, ophthalmically, orally, rectally, sublabially, sublingually, subcutaneously, topically, and transdermally; and/or (ii) with a frequency selected from the group consisting of four times daily, three times daily, twice daily, once daily, once every two days, once every three days, once every four days, once every five days, once every six days, once a week, once every eight days, once every nine days, once every ten days, once every two weeks, once every three weeks, and once a month; and/or (iii) at a dosage (based on subject weight) selected from the group consisting of 0.1 μg/kg, 0.5 μg/kg, 1 μg/kg, 1.5 μg/kg, 2.0 μg/kg, 2.5 μg/kg, 3.0 μg/kg, 3.5 μg/kg, 4.0 μg/kg, 4.5 μg/kg, 5.0 μg/kg, 10 μg/kg, 15 μg/kg, 20 μg/kg, 25 μg/kg, 30 μg/kg, 35 μg/kg, 40 μg/kg, 45 μg/kg, 50 μg/kg, 55 μg/kg, 60 μg/kg, 65 μg/kg, 70 μg/kg, 75 μg/kg, 80 μg/kg, 85 μg/kg, 90 μg/kg, 95 μg/kg, 100 μg/kg, 150 μg/kg, 200 μg/kg, 250 μg/kg, 300 μg/kg, 350 μg/kg, 400 μg/kg, 450 μg/kg, 500 μg/kg, 550 μg/kg, 600 μg/kg, 650 μg/kg, 700 μg/kg, 750 μg/kg, 800 μg/kg, 850 μg/kg, 900 μg/kg, 1 mg/kg, 1.5 mg/kg, 2.0 mg/kg, 2.5 mg/kg, 3 mg/kg, 3.5 mg/kg, 4.0 mg/kg, 4.5 mg/kg, 5 mg/kg, 5.5 mg/kg, 6 mg/kg, 6.5 mg/kg, 7 mg/kg, 7.5 mg/kg, 8 mg/kg, 8.5 mg/kg, 9 mg/kg, 9.5 mg/kg, 10 mg/kg 10.5 mg/kg, 11 mg/kg, 11.5 mg/kg, 12 mg/kg, 12.5 mg/kg, 13 mg/kg, 13.5 mg/kg, 14 mg/kg, 14.5 mg/kg, 15 mg/kg, 16 mg/kg, 17 mg/kg, 18 mg/kg, 19 mg/kg, 20 mg/kg, 21 mg/kg, 22 mg/kg, 23 mg/kg, 24 mg/kg, 25 mg/kg, 30 mg/kg, 35 mg/kg, 40 mg/kg, 45 mg/kg, 50 mg/kg, 55 mg/kg, 60 mg/kg, 65 mg/kg, 70 mg/kg, 75 mg/kg, 80 mg/kg, 85 mg/kg, 90 mg/kg, 95 mg/kg, 100 mg/kg, 105 mg/kg, 110 mg/kg, 115 mg/kg, 120 mg/kg, 125 mg/kg, 130 mg/kg, 135 mg/kg, 140 mg/kg, 145 mg/kg, 150 mg/kg, 155 mg/kg, 160 mg/kg, 165 mg/kg, 170 mg/kg, 175 mg/kg, 180 mg/kg, 185 mg/kg, 190 mg/kg, 195 mg/kg, 200 mg/kg, and any fraction or integer in between any two of the preceding amounts.

In some embodiments, an nNOS inhibitor composition comprising one or more siRNAs, may be administered as a suitable dosage form, as described herein, to treat, ameliorate, and/or reduce the severity of gastrointestinal cancer or one or more symptoms thereof in a gastrointestinal cancer subject. In certain embodiments, an nNOS inhibitor composition comprising one or more siRNAs selected from the group consisting of SEQ ID NO. 4, SEQ ID NO. 5, SEQ ID NO. 6, SEQ ID NO. 7, SEQ ID NO. 8, and any combination thereof, may be administered as a suitable dosage form, as described herein, to treat, ameliorate, and/or reduce the severity of gastrointestinal cancer or one or more symptoms thereof in a gastrointestinal cancer subject. In some embodiments, an nNOS inhibitor composition comprising one or more siRNAs selected from the group consisting of SEQ ID NO. 4, SEQ ID NO. 5, SEQ ID NO. 6, SEQ ID NO. 7, SEQ ID NO. 8, and any combination thereof, may be administered to a gastrointestinal cancer subject to treat, ameliorate, and/or reduce the severity of gastrointestinal cancer or one or more symptoms thereof in the gastrointestinal cancer subject: (i) via a route selected from the group consisting of buccally, inhalation, intraarterially, intraarticularly, intracardially, intracerebroventricularly, intradermally, intramedullary, intramuscularly, intraperitoneally, intrathecally, intravenously, intraventricularly, nasally, ophthalmically, orally, rectally, sublabially, sublingually, subcutaneously, topically, and transdermally; and/or (ii) with a frequency selected from the group consisting of four times daily, three times daily, twice daily, once daily, once every two days, once every three days, once every four days, once every five days, once every six days, once a week, once every eight days, once every nine days, once every ten days, once every two weeks, once every three weeks, and once a month; and/or (iii) at a dosage (based on subject weight) selected from the group consisting of 0.1 μg/kg, 0.5 μg/kg, 1 μg/kg, 1.5 μg/kg, 2.0 μg/kg, 2.5 μg/kg, 3.0 μg/kg, 3.5 μg/kg, 4.0 μg/kg, 4.5 μg/kg, 5.0 μg/kg, 10 μg/kg, 15 μg/kg, 20 μg/kg, 25 μg/kg, 30 μg/kg, 35 μg/kg, 40 μg/kg, 45 μg/kg, 50 μg/kg, 55 μg/kg, 60 μg/kg, 65 μg/kg, 70 μg/kg, 75 μg/kg, 80 μg/kg, 85 μg/kg, 90 μg/kg, 95 μg/kg, 100 μg/kg, 150 μg/kg, 200 μg/kg, 250 μg/kg, 300 μg/kg, 350 μg/kg, 400 μg/kg, 450 μg/kg, 500 μg/kg, 550 μg/kg, 600 μg/kg, 650 μg/kg, 700 μg/kg, 750 μg/kg, 800 μg/kg, 850 μg/kg, 900 μg/kg, 1 mg/kg, 1.5 mg/kg, 2.0 mg/kg, 2.5 mg/kg, 3 mg/kg, 3.5 mg/kg, 4.0 mg/kg, 4.5 mg/kg, 5 mg/kg, 5.5 mg/kg, 6 mg/kg, 6.5 mg/kg, 7 mg/kg, 7.5 mg/kg, 8 mg/kg, 8.5 mg/kg, 9 mg/kg, 9.5 mg/kg, 10 mg/kg 10.5 mg/kg, 11 mg/kg, 11.5 mg/kg, 12 mg/kg, 12.5 mg/kg, 13 mg/kg, 13.5 mg/kg, 14 mg/kg, 14.5 mg/kg, 15 mg/kg, 16 mg/kg, 17 mg/kg, 18 mg/kg, 19 mg/kg, 20 mg/kg, 21 mg/kg, 22 mg/kg, 23 mg/kg, 24 mg/kg, 25 mg/kg, 30 mg/kg, 35 mg/kg, 40 mg/kg, 45 mg/kg, 50 mg/kg, 55 mg/kg, 60 mg/kg, 65 mg/kg, 70 mg/kg, 75 mg/kg, 80 mg/kg, 85 mg/kg, 90 mg/kg, 95 mg/kg, 100 mg/kg, 105 mg/kg, 110 mg/kg, 115 mg/kg, 120 mg/kg, 125 mg/kg, 130 mg/kg, 135 mg/kg, 140 mg/kg, 145 mg/kg, 150 mg/kg, 155 mg/kg, 160 mg/kg, 165 mg/kg, 170 mg/kg, 175 mg/kg, 180 mg/kg, 185 mg/kg, 190 mg/kg, 195 mg/kg, 200 mg/kg, and any fraction or integer in between any two of the preceding amounts.

In some embodiments, an nNOS inhibitor composition comprising one or more shRNAs, may be administered as a suitable dosage form, as described herein, to treat, ameliorate, and/or reduce the severity of gastrointestinal cancer or one or more symptoms thereof in a gastrointestinal cancer subject. In certain embodiments, an nNOS inhibitor composition comprising one or more shRNAs selected from the group consisting of SEQ ID NO. 9, SEQ ID NO. 10, SEQ ID NO. 11, SEQ ID NO. 12, SEQ ID NO. 13, SEQ ID NO. 14, and any combination thereof, may be administered as a suitable dosage form, as described herein, to treat, ameliorate, and/or reduce the severity of gastrointestinal cancer or one or more symptoms thereof in a gastrointestinal cancer subject. In some embodiments, an nNOS inhibitor composition comprising one or more shRNAs selected from the group consisting of SEQ ID NO. 9, SEQ ID NO. 10, SEQ ID NO. 11, SEQ ID NO. 12, SEQ ID NO. 13, SEQ ID NO. 14, and any combination thereof, may be administered to a gastrointestinal cancer subject to treat, ameliorate, and/or reduce the severity of gastrointestinal cancer or one or more symptoms thereof in the gastrointestinal cancer subject: (i) via a route selected from the group consisting of buccally, inhalation, intraarterially, intraarticularly, intracardially, intracerebroventricularly, intradermally, intramedullary, intramuscularly, intraperitoneally, intrathecally, intravenously, intraventricularly, nasally, ophthalmically, orally, rectally, sublabially, sublingually, subcutaneously, topically, and transdermally; and/or (ii) with a frequency selected from the group consisting of four times daily, three times daily, twice daily, once daily, once every two days, once every three days, once every four days, once every five days, once every six days, once a week, once every eight days, once every nine days, once every ten days, once every two weeks, once every three weeks, and once a month; and/or (iii) at a dosage (based on subject weight) selected from the group consisting of 0.1 μg/kg, 0.5 μg/kg, 1 μg/kg, 1.5 μg/kg, 2.0 μg/kg, 2.5 μg/kg, 3.0 μg/kg, 3.5 μg/kg, 4.0 μg/kg, 4.5 μg/kg, 5.0 μg/kg, 10 μg/kg, 15 μg/kg, 20 μg/kg, 25 μg/kg, 30 μg/kg, 35 μg/kg, 40 μg/kg, 45 μg/kg, 50 μg/kg, 55 μg/kg, 60 μg/kg, 65 μg/kg, 70 μg/kg, 75 μg/kg, 80 μg/kg, 85 μg/kg, 90 μg/kg, 95 μg/kg, 100 μg/kg, 150 μg/kg, 200 μg/kg, 250 μg/kg, 300 μg/kg, 350 μg/kg, 400 μg/kg, 450 μg/kg, 500 μg/kg, 550 μg/kg, 600 μg/kg, 650 μg/kg, 700 μg/kg, 750 μg/kg, 800 μg/kg, 850 μg/kg, 900 μg/kg, 1 mg/kg, 1.5 mg/kg, 2.0 mg/kg, 2.5 mg/kg, 3 mg/kg, 3.5 mg/kg, 4.0 mg/kg, 4.5 mg/kg, 5 mg/kg, 5.5 mg/kg, 6 mg/kg, 6.5 mg/kg, 7 mg/kg, 7.5 mg/kg, 8 mg/kg, 8.5 mg/kg, 9 mg/kg, 9.5 mg/kg, 10 mg/kg 10.5 mg/kg, 11 mg/kg, 11.5 mg/kg, 12 mg/kg, 12.5 mg/kg, 13 mg/kg, 13.5 mg/kg, 14 mg/kg, 14.5 mg/kg, 15 mg/kg, 16 mg/kg, 17 mg/kg, 18 mg/kg, 19 mg/kg, 20 mg/kg, 21 mg/kg, 22 mg/kg, 23 mg/kg, 24 mg/kg, 25 mg/kg, 30 mg/kg, 35 mg/kg, 40 mg/kg, 45 mg/kg, 50 mg/kg, 55 mg/kg, 60 mg/kg, 65 mg/kg, 70 mg/kg, 75 mg/kg, 80 mg/kg, 85 mg/kg, 90 mg/kg, 95 mg/kg, 100 mg/kg, 105 mg/kg, 110 mg/kg, 115 mg/kg, 120 mg/kg, 125 mg/kg, 130 mg/kg, 135 mg/kg, 140 mg/kg, 145 mg/kg, 150 mg/kg, 155 mg/kg, 160 mg/kg, 165 mg/kg, 170 mg/kg, 175 mg/kg, 180 mg/kg, 185 mg/kg, 190 mg/kg, 195 mg/kg, 200 mg/kg, and any fraction or integer in between any two of the preceding amounts.

In some embodiments, an nNOS inhibitor composition comprising one or more miRNAs, may be administered as a suitable dosage form, as described herein, to treat, ameliorate, and/or reduce the severity of gastrointestinal cancer or one or more symptoms thereof in a gastrointestinal cancer subject. In certain embodiments, an nNOS inhibitor composition comprising one or more miRNAs selected from the group consisting of miR-146a, hsa-miR-146a-5p, hsa-miR-1273f, hsa-miR-143-3p, hsa-miR-147a, hsa-miR-3622a-5p, hsa-miR-3911, hsa-miR-4269, hsa-miR-4708-5p, hsa-miR-4710, hsa-miR-4770, hsa-miR-4792, hsa-miR-574-5p, hsa-miR-6088, hsa-miR-644a, hsa-miR-6715b-5p, hsa-miR-6768-5p, hsa-miR-6867-5p, hsa-miR-7855-5p, hsa-miR-8485, and any combination thereof, may be administered as a suitable dosage form, as described herein, to treat, ameliorate, and/or reduce the severity of gastrointestinal cancer or one or more symptoms thereof in a gastrointestinal cancer subject. In some embodiments, an nNOS inhibitor composition comprising one or more miRNAs selected from the group consisting of miR-146a, hsa-miR-146a-5p, hsa-miR-1273f, hsa-miR-143-3p, hsa-miR-147a, hsa-miR-3622a-5p, hsa-miR-3911, hsa-miR-4269, hsa-miR-4708-5p, hsa-miR-4710, hsa-miR-4770, hsa-miR-4792, hsa-miR-574-5p, hsa-miR-6088, hsa-miR-644a, hsa-miR-6715b-5p, hsa-miR-6768-5p, hsa-miR-6867-5p, hsa-miR-7855-5p, hsa-miR-8485, and any combination thereof, may be administered to a gastrointestinal cancer subject to treat, ameliorate, and/or reduce the severity of gastrointestinal cancer or one or more symptoms thereof in the gastrointestinal cancer subject: (i) via a route selected from the group consisting of buccally, inhalation, intraarterially, intraarticularly, intracardially, intracerebroventricularly, intradermally, intramedullary, intramuscularly, intraperitoneally, intrathecally, intravenously, intraventricularly, nasally, ophthalmically, orally, rectally, sublabially, sublingually, subcutaneously, topically, and transdermally; and/or (ii) with a frequency selected from the group consisting of four times daily, three times daily, twice daily, once daily, once every two days, once every three days, once every four days, once every five days, once every six days, once a week, once every eight days, once every nine days, once every ten days, once every two weeks, once every three weeks, and once a month; and/or (iii) at a dosage (based on subject weight) selected from the group consisting of 0.1 μg/kg, 0.5 μg/kg, 1 μg/kg, 1.5 μg/kg, 2.0 μg/kg, 2.5 μg/kg, 3.0 μg/kg, 3.5 μg/kg, 4.0 μg/kg, 4.5 μg/kg, 5.0 μg/kg, 10 μg/kg, 15 μg/kg, 20 μg/kg, 25 μg/kg, 30 μg/kg, 35 μg/kg, 40 μg/kg, 45 μg/kg, 50 μg/kg, 55 μg/kg, 60 μg/kg, 65 μg/kg, 70 μg/kg, 75 μg/kg, 80 μg/kg, 85 μg/kg, 90 μg/kg, 95 μg/kg, 100 μg/kg, 150 μg/kg, 200 μg/kg, 250 μg/kg, 300 μg/kg, 350 μg/kg, 400 μg/kg, 450 μg/kg, 500 μg/kg, 550 μg/kg, 600 μg/kg, 650 μg/kg, 700 μg/kg, 750 μg/kg, 800 μg/kg, 850 μg/kg, 900 μg/kg, 1 mg/kg, 1.5 mg/kg, 2.0 mg/kg, 2.5 mg/kg, 3 mg/kg, 3.5 mg/kg, 4.0 mg/kg, 4.5 mg/kg, 5 mg/kg, 5.5 mg/kg, 6 mg/kg, 6.5 mg/kg, 7 mg/kg, 7.5 mg/kg, 8 mg/kg, 8.5 mg/kg, 9 mg/kg, 9.5 mg/kg, 10 mg/kg 10.5 mg/kg, 11 mg/kg, 11.5 mg/kg, 12 mg/kg, 12.5 mg/kg, 13 mg/kg, 13.5 mg/kg, 14 mg/kg, 14.5 mg/kg, 15 mg/kg, 16 mg/kg, 17 mg/kg, 18 mg/kg, 19 mg/kg, 20 mg/kg, 21 mg/kg, 22 mg/kg, 23 mg/kg, 24 mg/kg, 25 mg/kg, 30 mg/kg, 35 mg/kg, 40 mg/kg, 45 mg/kg, 50 mg/kg, 55 mg/kg, 60 mg/kg, 65 mg/kg, 70 mg/kg, 75 mg/kg, 80 mg/kg, 85 mg/kg, 90 mg/kg, 95 mg/kg, 100 mg/kg, 105 mg/kg, 110 mg/kg, 115 mg/kg, 120 mg/kg, 125 mg/kg, 130 mg/kg, 135 mg/kg, 140 mg/kg, 145 mg/kg, 150 mg/kg, 155 mg/kg, 160 mg/kg, 165 mg/kg, 170 mg/kg, 175 mg/kg, 180 mg/kg, 185 mg/kg, 190 mg/kg, 195 mg/kg, 200 mg/kg, and any fraction or integer in between any two of the preceding amounts.

In some embodiments, an nNOS inhibitor composition comprising one or more CRISPR/Cas systems, may be administered as a suitable dosage form, as described herein, to treat, ameliorate, and/or reduce the severity of gastrointestinal cancer or one or more symptoms thereof in a gastrointestinal cancer subject. In certain embodiments, an nNOS inhibitor composition comprising one or more CRISPR/Cas systems comprising an endonuclease selected from the group consisting of Cas1, Cas2, Cas3, Cas4, Cas5, Cas6, Cas7, Cas8a, Cas8b, Cas8c, Cas9, Cas10, Cas10d, Cas12, Cas12a, Cas12b, Cas12c, Cas12d, Cas12e, Cas12f, Cas12g, Cas12h, Cas12i, Cas12k, C2c4, C2c8, C2c9, Cas13, Cas13a, Cas13b, Cas13c, Cas13d, Cas13x.1, Cse1, Cse2, Csy1, Csy2, Csy3, GSU0054, Csm2, Cmr5, Csx10, Csx11, Csf1, and Csn2, may be administered as a suitable dosage form, as described herein, to treat, ameliorate, and/or reduce the severity of gastrointestinal cancer or one or more symptoms thereof in a gastrointestinal cancer subject. In some embodiments, an nNOS inhibitor composition comprising one or more CRISPR/Cas systems comprising an endonuclease selected from the group consisting of Cas1, Cas2, Cas3, Cas4, Cas5, Cas6, Cas7, Cas8a, Cas8b, Cas8c, Cas9, Cas10, Cas10d, Cas12, Cas12a, Cas12b, Cas12c, Cas12d, Cas12e, Cas12f, Cas12g, Cas12h, Cas12i, Cas12k, C2c4, C2c8, C2c9, Cas13, Cas13a, Cas13b, Cas13c, Cas13d, Cas13x.1, Cse1, Cse2, Csy1, Csy2, Csy3, GSU0054, Csm2, Cmr5, Csx10, Csx11, Csf1, and Csn2, may be administered to a gastrointestinal cancer subject to treat, ameliorate, and/or reduce the severity of gastrointestinal cancer or one or more symptoms thereof in the gastrointestinal cancer subject: (i) via a route selected from the group consisting of buccally, inhalation, intraarterially, intraarticularly, intracardially, intracerebroventricularly, intradermally, intramedullary, intramuscularly, intraperitoneally, intrathecally, intravenously, intraventricularly, nasally, ophthalmically, orally, rectally, sublabially, sublingually, subcutaneously, topically, and transdermally; and/or (ii) with a frequency selected from the group consisting of four times daily, three times daily, twice daily, once daily, once every two days, once every three days, once every four days, once every five days, once every six days, once a week, once every eight days, once every nine days, once every ten days, once every two weeks, once every three weeks, and once a month; and/or (iii) at a dosage (based on subject weight) selected from the group consisting of 0.1 μg/kg, 0.5 μg/kg, 1 μg/kg, 1.5 μg/kg, 2.0 μg/kg, 2.5 μg/kg, 3.0 μg/kg, 3.5 μg/kg, 4.0 μg/kg, 4.5 μg/kg, 5.0 μg/kg, 10 μg/kg, 15 μg/kg, 20 μg/kg, 25 μg/kg, 30 μg/kg, 35 μg/kg, 40 μg/kg, 45 μg/kg, 50 μg/kg, 55 μg/kg, 60 μg/kg, 65 μg/kg, 70 μg/kg, 75 μg/kg, 80 μg/kg, 85 μg/kg, 90 μg/kg, 95 μg/kg, 100 μg/kg, 150 μg/kg, 200 μg/kg, 250 μg/kg, 300 μg/kg, 350 μg/kg, 400 μg/kg, 450 μg/kg, 500 μg/kg, 550 μg/kg, 600 μg/kg, 650 μg/kg, 700 μg/kg, 750 μg/kg, 800 μg/kg, 850 μg/kg, 900 μg/kg, 1 mg/kg, 1.5 mg/kg, 2.0 mg/kg, 2.5 mg/kg, 3 mg/kg, 3.5 mg/kg, 4.0 mg/kg, 4.5 mg/kg, 5 mg/kg, 5.5 mg/kg, 6 mg/kg, 6.5 mg/kg, 7 mg/kg, 7.5 mg/kg, 8 mg/kg, 8.5 mg/kg, 9 mg/kg, 9.5 mg/kg, 10 mg/kg 10.5 mg/kg, 11 mg/kg, 11.5 mg/kg, 12 mg/kg, 12.5 mg/kg, 13 mg/kg, 13.5 mg/kg, 14 mg/kg, 14.5 mg/kg, 15 mg/kg, 16 mg/kg, 17 mg/kg, 18 mg/kg, 19 mg/kg, 20 mg/kg, 21 mg/kg, 22 mg/kg, 23 mg/kg, 24 mg/kg, 25 mg/kg, 30 mg/kg, 35 mg/kg, 40 mg/kg, 45 mg/kg, 50 mg/kg, 55 mg/kg, 60 mg/kg, 65 mg/kg, 70 mg/kg, 75 mg/kg, 80 mg/kg, 85 mg/kg, 90 mg/kg, 95 mg/kg, 100 mg/kg, 105 mg/kg, 110 mg/kg, 115 mg/kg, 120 mg/kg, 125 mg/kg, 130 mg/kg, 135 mg/kg, 140 mg/kg, 145 mg/kg, 150 mg/kg, 155 mg/kg, 160 mg/kg, 165 mg/kg, 170 mg/kg, 175 mg/kg, 180 mg/kg, 185 mg/kg, 190 mg/kg, 195 mg/kg, 200 mg/kg, and any fraction or integer in between any two of the preceding amounts.

In some embodiments, an nNOS inhibitor composition comprising one or more antibodies, may be administered as a suitable dosage form, as described herein, to treat, ameliorate, and/or reduce the severity of gastrointestinal cancer or one or more symptoms thereof in a gastrointestinal cancer subject. In certain embodiments, an nNOS inhibitor composition comprising one or more antibodies selected from the group consisting of 37-2800 (from ThermoFisher), EP1855Y (from abeam), EPR 24351-6 (from abeam), and any combination thereof, may be administered as a suitable dosage form, as described herein, to treat, ameliorate, and/or reduce the severity of gastrointestinal cancer or one or more symptoms thereof in a gastrointestinal cancer subject. In some embodiments, an nNOS inhibitor composition comprising one or more antibodies selected from the group consisting of 37-2800 (from ThermoFisher), EP1855Y (from abeam), EPR 24351-6 (from abeam), and any combination thereof, may be administered to a gastrointestinal cancer subject to treat, ameliorate, and/or reduce the severity of gastrointestinal cancer or one or more symptoms thereof in the gastrointestinal cancer subject: (i) via a route selected from the group consisting of buccally, inhalation, intraarterially, intraarticularly, intracardially, intracerebroventricularly, intradermally, intramedullary, intramuscularly, intraperitoneally, intrathecally, intravenously, intraventricularly, nasally, ophthalmically, orally, rectally, sublabially, sublingually, subcutaneously, topically, and transdermally; and/or (ii) with a frequency selected from the group consisting of four times daily, three times daily, twice daily, once daily, once every two days, once every three days, once every four days, once every five days, once every six days, once a week, once every eight days, once every nine days, once every ten days, once every two weeks, once every three weeks, and once a month; and/or (iii) at a dosage (based on subject weight) selected from the group consisting of 0.1 μg/kg, 0.5 μg/kg, 1 μg/kg, 1.5 μg/kg, 2.0 μg/kg, 2.5 μg/kg, 3.0 μg/kg, 3.5 μg/kg, 4.0 μg/kg, 4.5 μg/kg, 5.0 μg/kg, 10 μg/kg, 15 μg/kg, 20 μg/kg, 25 μg/kg, 30 μg/kg, 35 μg/kg, 40 μg/kg, 45 μg/kg, 50 μg/kg, 55 μg/kg, 60 μg/kg, 65 μg/kg, 70 μg/kg, 75 μg/kg, 80 μg/kg, 85 μg/kg, 90 μg/kg, 95 μg/kg, 100 μg/kg, 150 μg/kg, 200 μg/kg, 250 μg/kg, 300 μg/kg, 350 μg/kg, 400 μg/kg, 450 μg/kg, 500 μg/kg, 550 μg/kg, 600 μg/kg, 650 μg/kg, 700 μg/kg, 750 μg/kg, 800 μg/kg, 850 μg/kg, 900 μg/kg, 1 mg/kg, 1.5 mg/kg, 2.0 mg/kg, 2.5 mg/kg, 3 mg/kg, 3.5 mg/kg, 4.0 mg/kg, 4.5 mg/kg, 5 mg/kg, 5.5 mg/kg, 6 mg/kg, 6.5 mg/kg, 7 mg/kg, 7.5 mg/kg, 8 mg/kg, 8.5 mg/kg, 9 mg/kg, 9.5 mg/kg, 10 mg/kg 10.5 mg/kg, 11 mg/kg, 11.5 mg/kg, 12 mg/kg, 12.5 mg/kg, 13 mg/kg, 13.5 mg/kg, 14 mg/kg, 14.5 mg/kg, 15 mg/kg, 16 mg/kg, 17 mg/kg, 18 mg/kg, 19 mg/kg, 20 mg/kg, 21 mg/kg, 22 mg/kg, 23 mg/kg, 24 mg/kg, 25 mg/kg, 30 mg/kg, 35 mg/kg, 40 mg/kg, 45 mg/kg, 50 mg/kg, 55 mg/kg, 60 mg/kg, 65 mg/kg, 70 mg/kg, 75 mg/kg, 80 mg/kg, 85 mg/kg, 90 mg/kg, 95 mg/kg, 100 mg/kg, 105 mg/kg, 110 mg/kg, 115 mg/kg, 120 mg/kg, 125 mg/kg, 130 mg/kg, 135 mg/kg, 140 mg/kg, 145 mg/kg, 150 mg/kg, 155 mg/kg, 160 mg/kg, 165 mg/kg, 170 mg/kg, 175 mg/kg, 180 mg/kg, 185 mg/kg, 190 mg/kg, 195 mg/kg, 200 mg/kg, and any fraction or integer in between any two of the preceding amounts.

In some embodiments, an nNOS inhibitor composition comprising one or more amino acid-based compounds, may be administered as a suitable dosage form, as described herein, to treat, ameliorate, and/or reduce the severity of gastrointestinal cancer or one or more symptoms thereof in a gastrointestinal cancer subject. In certain embodiments, an nNOS inhibitor composition comprising one or more amino acid-compounds that bind an active site of nNOS selected from the group consisting of Phenylalanine 589, Tryptophan 592, Tyrosine 593, Glutamate 597, Aspartate 602, Arginine 601, Methionine 341, Histidine 342, and Tyrosine711, may be administered as a suitable dosage form, as described herein, to treat, ameliorate, and/or reduce the severity of gastrointestinal cancer or one or more symptoms thereof in a gastrointestinal cancer subject. In some embodiments, an nNOS inhibitor composition comprising one or more amino acid-compounds that bind an active site of nNOS selected from the group consisting of Phenylalanine 589, Tryptophan 592, Tyrosine 593, Glutamate 597, Aspartate 602, Arginine 601, Methionine 341, Histidine 342, and Tyrosine711, may be administered to a gastrointestinal cancer subject to treat, ameliorate, and/or reduce the severity of gastrointestinal cancer or one or more symptoms thereof in the gastrointestinal cancer subject: (i) via a route selected from the group consisting of buccally, inhalation, intraarterially, intraarticularly, intracardially, intracerebroventricularly, intradermally, intramedullary, intramuscularly, intraperitoneally, intrathecally, intravenously, intraventricularly, nasally, ophthalmically, orally, rectally, sublabially, sublingually, subcutaneously, topically, and transdermally; and/or (ii) with a frequency selected from the group consisting of four times daily, three times daily, twice daily, once daily, once every two days, once every three days, once every four days, once every five days, once every six days, once a week, once every eight days, once every nine days, once every ten days, once every two weeks, once every three weeks, and once a month; and/or (iii) at a dosage (based on subject weight) selected from the group consisting of 0.1 μg/kg, 0.5 μg/kg, 1 μg/kg, 1.5 μg/kg, 2.0 μg/kg, 2.5 μg/kg, 3.0 μg/kg, 3.5 μg/kg, 4.0 μg/kg, 4.5 μg/kg, 5.0 μg/kg, 10 μg/kg, 15 μg/kg, 20 μg/kg, 25 μg/kg, 30 μg/kg, 35 μg/kg, 40 μg/kg, 45 μg/kg, 50 μg/kg, 55 μg/kg, 60 μg/kg, 65 μg/kg, 70 μg/kg, 75 μg/kg, 80 μg/kg, 85 μg/kg, 90 μg/kg, 95 μg/kg, 100 μg/kg, 150 μg/kg, 200 μg/kg, 250 μg/kg, 300 μg/kg, 350 μg/kg, 400 μg/kg, 450 μg/kg, 500 μg/kg, 550 μg/kg, 600 μg/kg, 650 μg/kg, 700 μg/kg, 750 μg/kg, 800 μg/kg, 850 μg/kg, 900 μg/kg, 1 mg/kg, 1.5 mg/kg, 2.0 mg/kg, 2.5 mg/kg, 3 mg/kg, 3.5 mg/kg, 4.0 mg/kg, 4.5 mg/kg, 5 mg/kg, 5.5 mg/kg, 6 mg/kg, 6.5 mg/kg, 7 mg/kg, 7.5 mg/kg, 8 mg/kg, 8.5 mg/kg, 9 mg/kg, 9.5 mg/kg, 10 mg/kg 10.5 mg/kg, 11 mg/kg, 11.5 mg/kg, 12 mg/kg, 12.5 mg/kg, 13 mg/kg, 13.5 mg/kg, 14 mg/kg, 14.5 mg/kg, 15 mg/kg, 16 mg/kg, 17 mg/kg, 18 mg/kg, 19 mg/kg, 20 mg/kg, 21 mg/kg, 22 mg/kg, 23 mg/kg, 24 mg/kg, 25 mg/kg, 30 mg/kg, 35 mg/kg, 40 mg/kg, 45 mg/kg, 50 mg/kg, 55 mg/kg, 60 mg/kg, 65 mg/kg, 70 mg/kg, 75 mg/kg, 80 mg/kg, 85 mg/kg, 90 mg/kg, 95 mg/kg, 100 mg/kg, 105 mg/kg, 110 mg/kg, 115 mg/kg, 120 mg/kg, 125 mg/kg, 130 mg/kg, 135 mg/kg, 140 mg/kg, 145 mg/kg, 150 mg/kg, 155 mg/kg, 160 mg/kg, 165 mg/kg, 170 mg/kg, 175 mg/kg, 180 mg/kg, 185 mg/kg, 190 mg/kg, 195 mg/kg, 200 mg/kg, and any fraction or integer in between any two of the preceding amounts.

In some embodiments, a gastrointestinal cancer subject, as described herein, may be a female child or a male child. In certain embodiments, a gastrointestinal cancer subject, as described herein, may be a female adult or a male adult.

In certain embodiments, a gastrointestinal cancer subject, as described herein, may have a gastrointestinal cancer occurring and/or originating in the anus, such as, for example, an adenocarcinoma, a basal cell carcinoma, a carcinoma in situ, a cloacogenic carcinoma, a gastrointestinal stromal tumor, a lymphoma, a melanoma, a poorly differentiated neuroendocrine tumor, or a squamous cell carcinoma.

In some embodiments, a gastrointestinal cancer subject, as described herein, may have a gastrointestinal cancer occurring and/or originating in the bile duct, such as, for example, an adenocarcinoma, a distal bile duct cancer, an extrahepatic bile duct cancer, a hilar bile duct cancer, an intrahepatic bile duct cancer, an intrahepatic cholangiocarcinoma, a lymphoma, a perihilar bile duct cancer, a sarcoma, or a squamous cell carcinoma.

In certain embodiments, a gastrointestinal cancer subject, as described herein, may have a gastrointestinal cancer occurring and/or originating in the colon, such as, for example, an adenocarcinoma, an adenoma, a carcinoid tumor, a familial adenomatous polyposis, a gastrointestinal stromal tumor, a leiomyosarcoma, a melanoma, a mucinous adenocarcinoma, a neuroendocrine tumor of the gastrointestinal tract, a primary colorectal lymphoma, a signet ring adenocarcinoma, or a squamous cell carcinoma.

In some embodiments, a gastrointestinal cancer subject, as described herein, may have a gastrointestinal cancer occurring and/or originating in the esophagus/gastroesophageal junction, such as, for example, an adenocarcinoma, a lymphoma, a neuroendocrine tumor, a melanoma, a sarcoma, a small cell carcinoma, or a squamous cell carcinoma.

In certain embodiments, a gastrointestinal cancer subject, as described herein, may have a gastrointestinal cancer occurring and/or originating in the gallbladder, such as, for example, an adenocarcinoma, an adenosquamous carcinoma, a carcinosarcoma, a lymphoma, a melanoma, a mucinous adenocarcinoma, a neuroendocrine tumor, a non-papillary adenocarcinoma, a papillary adenocarcinoma, a sarcoma, a small cell carcinoma, or a squamous cell carcinoma.

In some embodiments, a gastrointestinal cancer subject, as described herein, may have a gastrointestinal cancer occurring and/or originating in the liver, such as, for example, an adenoma, an angiosarcoma, cholangiocarcinoma, an extrahepatic cholangiocarcinoma, fibrolamellar hepatocellular carcinoma, a focal nodular hyperplasia, a haemangioma, a hepatoblastoma, a hepatocellular carcinoma, an intrahepatic cholangiocarcinoma, or a squamous cell carcinoma.

In certain embodiments, a gastrointestinal cancer subject, as described herein, may have a gastrointestinal cancer occurring and/or originating in the renal system, such as, for example, an angiomyolipoma, a chromophobe carcinoma, a clear cell carcinoma, a clear cell papillary, a collecting duct carcinoma, a medullary carcinoma, a multilocular cystic carcinoma, a mucinous tubular carcinoma, a nephroblastoma, a neuroblastoma-associated renal cell carcinoma, a papillary carcinoma type I, a papillary carcinoma type II, a renal cell adenoma, a renal cell carcinoma, a renal oncocytoma, a renal sarcoma, a sarcomatoid, a spindle cell carcinoma, a transitional cell carcinoma, an unclassified carcinoma, a urothelial carcinoma, or a Wilms' tumor.

In some embodiments, a gastrointestinal cancer subject, as described herein, may have a gastrointestinal cancer occurring and/or originating in the small intestine, such as, for example, an adenocarcinoma, a carcinoid tumor, a gastrinoma, a gastrointestinal stromal tumor, a leiomyosarcoma, a lymphoma, or a sarcoma.

In certain embodiments, a gastrointestinal cancer subject, as described herein, may have a gastrointestinal cancer occurring and/or originating in the stomach (gastric cancer), such as, for example, an adenocarcinoma of the mucosa, an adenocarcinoma of the muscularis propria, an adenocarcinoma of the serosa, an adenocarcinoma of the submucosa, an adenocarcinoma of the subserosa, a diffuse adenocarcinoma, a gastrointestinal stromal tumor, an intestinal adenocarcinoma, an intestinal adenoma, a neuroendocrine carcinoid tumor, a neuroendocrine tumor, or a primary gastric lymphoma.

In some embodiments, a gastrointestinal cancer subject, as described herein, may have an abnormal expression of one or more antigens compared to an equivalent subject that does not have gastrointestinal cancer. As used herein, the term “equivalent subject,” refers to a subject of the same sex (male/female) and age range (child/adult) of the gastrointestinal cancer subject. For purposes of disclosure, the term “antigen,” when used with respect to abnormal expression, may define one or more CD antigens, such as, for example, CD15, CD44, CD70, CD133, as well as any CD antigen that has an abnormal expression in a gastrointestinal cancer disclosed herein that would be readily envisaged by one skilled in the art. In certain embodiments, abnormal expression of an antigen may define an abnormal expression of CD15, an abnormal expression of CD44, an abnormal expression of CD70, an abnormal expression of CD133, an abnormal expression of a CD antigen that has an abnormal expression in a gastrointestinal cancer disclosed herein that would be readily envisaged by one skilled in the art, and any combination thereof. In some embodiments, an “abnormal expression,” can refer to a reduced expression of an antigen, as described herein. In certain embodiments, an “abnormal expression,” can refer to an overexpression of an antigen, as described herein. Whether “abnormal expression,” refers to a reduced expression of an antigen or an overexpression of an antigen would be immediately envisaged by the skilled artisan when viewing the term in the context of the particular antigen disclosed, as well as the gastrointestinal cancer to which the abnormal expression of antigen refers to. For example, in the context of CD70 and renal cell carcinoma, one skilled in the art would immediately envisage that “abnormal expression” refers to an overexpression of CD70. Treatment, amelioration, and/or reduction in the severity of a gastrointestinal cancer or one or more symptoms thereof may be characterized by a restoration to a normal level of expression of one or more antigens from their abnormal state, as described herein. For example, in the context of CD70 and renal cell carcinoma, treatment, amelioration, and/or reduction in the severity of a gastrointestinal cancer or one or more symptoms thereof may be characterized by a reduced expression of CD70.

In certain embodiments, a gastrointestinal cancer subject, as described herein, may have an abnormal expression of one or more proteins compared to an equivalent subject that does not have gastrointestinal cancer. For purposes of disclosure, the term “protein,” when used with respect to abnormal expression, may define one or more proteins and/or enzymes, such as, for example, ALDH1A3, mTOR, p-mTOR, TSC-2, AKT, pAKT, RPS6, p-RPS6, Syp, VEGF, PIGF, as well as any protein or enzyme that has an abnormal expression in a gastrointestinal cancer disclosed herein that would be readily envisaged by one skilled in the art. In certain embodiments, abnormal expression of a protein may define an abnormal expression of ALDH1A3, an abnormal expression of mTOR, an abnormal expression of p-mTOR, an abnormal expression of TSC-2, an abnormal expression of AKT, an abnormal expression of pAKT, an abnormal expression of RPS6, an abnormal expression of p-RPS6, an abnormal expression of Syp, an abnormal expression of VEGF, an abnormal expression of PIGF, an abnormal expression of a protein or enzyme that has an abnormal expression in a gastrointestinal cancer disclosed herein that would be readily envisaged by one skilled in the art, and any combination thereof. In some embodiments, an “abnormal expression,” can refer to a reduced expression of a protein, as described herein. In certain embodiments, an “abnormal expression,” can refer to an overexpression of a protein, as described herein. Whether “abnormal expression,” refers to a reduced expression of a protein or an overexpression of a protein would be immediately envisaged by the skilled artisan when viewing the term in the context of the particular protein disclosed, as well as the gastrointestinal cancer to which the abnormal expression of protein refers to. For example, in the context of VEGF and gastric cancers, one skilled in the art would immediately envisage that “abnormal expression” refers to an overexpression of VEGF. Treatment, amelioration, and/or reduction in the severity of a gastrointestinal cancer or one or more symptoms thereof may be characterized by a restoration to a normal level of expression of one or more proteins from their abnormal state, as described herein. For example, in the context of VEGF and gastric cancers, treatment, amelioration, and/or reduction in the severity of a gastrointestinal cancer or one or more symptoms thereof may be characterized by a reduced expression of VEGF.

In certain embodiments, a gastrointestinal cancer subject, as described herein, may have an increased expression of one or more NOS isoforms compared to an equivalent subject that does not have gastrointestinal cancer. For purposes of disclosure, the term “NOS isoform,” refers to nNOS, iNOS, and/or eNOS. In certain embodiments, a gastrointestinal cancer subject, as described herein, may have an increased expression of nNOS compared to an equivalent subject that does not have gastrointestinal cancer. In some embodiments, a gastrointestinal cancer subject, as described herein, may have an increased expression of iNOS compared to an equivalent subject that does not have gastrointestinal cancer. In certain embodiments, a gastrointestinal cancer subject, as described herein, may have an increased expression of eNOS compared to an equivalent subject that does not have gastrointestinal cancer. In some embodiments, a gastrointestinal cancer subject, as described herein, may have an increased expression of nNOS and iNOS compared to an equivalent subject that does not have gastrointestinal cancer. In certain embodiments, a gastrointestinal cancer subject, as described herein, may have an increased expression of nNOS and eNOS compared to an equivalent subject that does not have gastrointestinal cancer. In some embodiments, a gastrointestinal cancer subject, as described herein, may have an increased expression of iNOS and eNOS compared to an equivalent subject that does not have gastrointestinal cancer. In certain embodiments, a gastrointestinal cancer subject, as described herein, may have an increased expression of nNOS, iNOS, and eNOS compared to an equivalent subject that does not have gastrointestinal cancer. Whether a “NOS isoform,” has an increased expression would be immediately envisaged by the skilled artisan when viewing the term in the context of the particular NOS isoform disclosed, as well as the gastrointestinal cancer to which the increased expression of NOS isoform refers to. For example, in the context of renal cell carcinoma, one skilled in the art would immediately envisage that iNOS has an increased expression. Treatment, amelioration, and/or reduction in the severity of a gastrointestinal cancer or one or more symptoms thereof may be characterized by a reduced expression of one or more NOS isoforms from their increased state, as described herein. For example, in the context of renal cell carcinoma, treatment, amelioration, and/or reduction in the severity of a gastrointestinal cancer or one or more symptoms thereof may be characterized by a reduced expression of iNOS. For purposes of disclosure, reduction/inhibition of the expression of one or more NOS isoforms may be characterized by a reduction in expression of one or more NOS isoforms by about at least 10%, about at least 20%, about at least 30%, or about at least 40%. In some embodiments, reduction/inhibition of the expression of one or more NOS isoforms may further be characterized by a lack reduction of other, non-implicated NOS isoforms, such as, for example, no more than about 10%, no more than about 5%, no more than about 2%, or no more than about 1% reduction/inhibition of the expression of non-implicated NOS isoforms. As used herein, the term “non-implicated NOS isoforms,” refers to NOS isoforms that do not have an increased expression in a particular gastrointestinal cancer. By way of example, in the context of renal cell carcinoma, if iNOS has an increased expression, nNOS and eNOS would define “non-implicated” NOS isoforms. Whether a NOS isoform is a “non-implicated NOS isoform,” would be immediately envisaged by the skilled artisan when viewing the term in the context of the particular NOS isoform disclosed, as well as the gastrointestinal cancer.

In some embodiments, a gastrointestinal cancer subject, as described herein, may have an elevated level of one or more NO-indicators compared to an equivalent subject that does not have gastrointestinal cancer. As used herein, the term “NO-indicator,” can refer to 3-nitrotyrosine (3-Ntyr), NADPH-diaphorase, total nitrite concentration, total nitrate concentration, total nitrite and nitrate concentration, as well as any NO-indicator that has an elevated level in a gastrointestinal cancer disclosed herein that would be readily envisaged by one skilled in the art. For example, in the context of renal cell carcinoma, one skilled in the art would immediately envisage that 3-Ntyr can be present at an elevated level. Treatment, amelioration, and/or reduction in the severity of a gastrointestinal cancer or one or more symptoms thereof may be characterized by a reduction in the level of one or more NO-indicators. For example, in the context of renal cell carcinoma, treatment, amelioration, and/or reduction in the severity of a gastrointestinal cancer or one or more symptoms thereof may be characterized by a reduction in the level of 3-Ntyr.

In certain embodiments, a NOS inhibitor composition, as described herein, may be administered as a suitable dosage form, as described herein, to treat, ameliorate, and/or reduce the severity of a gastrointestinal cancer or one or more symptoms thereof, as an alternative to a conventional therapy for treating, ameliorating, and/or reducing the severity of a gastrointestinal cancer or one or more symptoms thereof. For purposes of disclosure, a “conventional therapy,” as described herein, may refer to a treatment that would be recommended by one skilled in the art to treat a gastrointestinal cancer, and may define one or more of radiation, chemotherapy, immunotherapy, cell-based therapy, gene therapy, and the like—one skilled in the art would immediately envisage a “conventional therapy” based on the particular gastrointestinal cancer disclosed, as well as characteristics of a gastrointestinal cancer subject, such as, for example, the subject's sex, age, stage of the gastrointestinal cancer, genotype of the gastrointestinal cancer, and the like. Treatment, amelioration, and/or reduction in the severity of a gastrointestinal cancer or one or more symptoms thereof may be characterized by an improved outcome compared to a conventional therapy, as described herein. For purposes of the disclosure, an “improved outcome” may define any one of the following: (i) a larger reduction in tumor volume and/or mass compared to a subject administered an equivalent dose of a conventional therapy; (ii) an improvement in the expected rate of survival of a subject, at, for example, 1 month, 3 months, 6 months, 9 months, 1 year, 2 years, 3 years, 5 years, 10 years, or more, compared to the expected rate of survival of a subject administered an equivalent dose of a conventional therapy; (iii) a reduction in the prevalence of side effects resulting from the NOS inhibitor composition compared to a subject administered an equivalent dose of a conventional therapy; (iv) a reduction in the severity of side effects resulting from the NOS inhibitor composition compared to a subject administered an equivalent dose of a conventional therapy; (v) a reduction in the dosage required to achieve an equivalent reduction in tumor volume compared to a subject administered a conventional therapy; (vi) a reduction in the dosage required to achieve an equivalent expected rate of survival of a subject compared to a subject administered a conventional therapy; (vii) a reduction in the likelihood of a recurrence of the gastrointestinal cancer occurring in a subject compared to a subject administered an equivalent dose of a conventional therapy; (viii) a reduction in the likelihood of a metastasis occurring in a subject compared to a subject administered an equivalent dose of a conventional therapy; (ix) a reduction in the time required to be at a medical facility for treatment compared to a subject administered an equivalent dose of a conventional therapy; (x) a reduction in the cost associated with treatment compared to a subject administered an equivalent dose of a conventional therapy; as well as any other improved outcome not expressly recited that would be readily envisaged by one skilled in the art.

In some embodiments, a NOS inhibitor composition, as described herein, may be administered as a suitable dosage form, as described herein, to treat, ameliorate, and/or reduce the severity of a gastrointestinal cancer or one or more symptoms thereof, wherein the NOS inhibitor composition comprises a first chemical NOS inhibitor and a second chemical NOS inhibitor. In certain embodiments, the first chemical NOS inhibitor may be administered concurrently with the second chemical NOS inhibitor. In some embodiments, the first chemical NOS inhibitor may be administered prior to the second chemical NOS inhibitor. In certain embodiments, the first chemical NOS inhibitor may be administered subsequently to the second chemical NOS inhibitor. Treatment, amelioration, and/or reduction in the severity of a gastrointestinal cancer or one or more symptoms thereof may be characterized by an improved outcome compared an alternative treatment. As used herein, the term “alternative treatment,” may define one or more of a conventional therapy, as described herein, a NOS inhibitor composition comprising a single chemical NOS inhibitor, as described herein, and a NOS inhibitor composition comprising a single biological NOS inhibitor, as described herein. For purposes of the disclosure, an “improved outcome” may define any one of the following: (i) a larger reduction in tumor volume and/or mass compared an alternative treatment; (ii) an improvement in the expected rate of survival of a subject, at, for example, 1 month, 3 months, 6 months, 9 months, 1 year, 2 years, 3 years, 5 years, 10 years, or more, compared to the expected rate of survival of a subject administered an alternative treatment; (iii) a reduction in the prevalence of side effects resulting from the NOS inhibitor composition comprising at least two chemical NOS inhibitors compared to a subject administered an alternative treatment; (iv) a reduction in the severity of side effects resulting from the NOS inhibitor composition comprising at least two chemical NOS inhibitors compared to a subject administered an alternative treatment; (v) a reduction in the dosage required to achieve an equivalent reduction in tumor volume compared to a subject administered an alternative treatment; (vi) a reduction in the dosage required to achieve an equivalent expected rate of survival of a subject compared to a subject administered an alternative treatment; (vii) a reduction in the likelihood of a recurrence of the gastrointestinal cancer in a subject compared to a subject administered an alternative treatment; (viii) a reduction in the likelihood of a metastasis occurring in a subject compared to a subject administered an alternative treatment; (ix) a reduction in the time required to be at a medical facility for treatment compared to a subject administered an alternative treatment; (x) a reduction in the cost associated with treatment compared to a subject administered an alternative treatment; as well as any other improved outcome not expressly recited that would be readily envisaged by one skilled in the art.

In certain embodiments, a NOS inhibitor composition, as described herein, may be used to treat, ameliorate, and/or reduce the severity of a gastrointestinal cancer or one or more symptoms thereof, wherein the NOS inhibitor composition comprises at least one chemical NOS inhibitor and at least one biological NOS inhibitor. In some embodiments, the at least one chemical NOS inhibitor may be administered concurrently with the at least one biological NOS inhibitor. In certain embodiments, the at least one chemical NOS inhibitor may be administered prior to the at least one biological NOS inhibitor. In some embodiments, the at least one chemical NOS inhibitor may be administered subsequently to the at least one biological NOS inhibitor. Treatment, amelioration, and/or reduction in the severity of a gastrointestinal cancer or one or more symptoms thereof may be characterized by an improved outcome compared an alternative treatment. As used herein, the term “alternative treatment,” may define one or more of a conventional therapy, as described herein, a NOS inhibitor composition comprising a single chemical NOS inhibitor, as described herein, and a NOS inhibitor composition comprising a single biological NOS inhibitor, as described herein. For purposes of the disclosure, an “improved outcome” may define any one of the following: (i) a larger reduction in tumor volume and/or mass compared an alternative treatment; (ii) an improvement in the expected rate of survival of a subject, at, for example, 1 month, 3 months, 6 months, 9 months, 1 year, 2 years, 3 years, 5 years, 10 years, or more, compared to the expected rate of survival of a subject administered an alternative treatment; (iii) a reduction in the prevalence of side effects resulting from the NOS inhibitor composition comprising at least one chemical NOS inhibitor and at least one biological NOS inhibitor compared to a subject administered an alternative treatment; (iv) a reduction in the severity of side effects resulting from the NOS inhibitor composition comprising at least one chemical NOS inhibitor and at least one biological NOS inhibitor compared to a subject administered an alternative treatment; (v) a reduction in the dosage required to achieve an equivalent reduction in tumor volume compared to a subject administered an alternative treatment; (vi) a reduction in the dosage required to achieve an equivalent expected rate of survival of a subject compared to a subject administered an alternative treatment; (vii) a reduction in the likelihood of a recurrence of the gastrointestinal cancer in a subject compared to a subject administered an alternative treatment; (viii) a reduction in the likelihood of a metastasis occurring in a subject compared to a subject administered an alternative treatment; (ix) a reduction in the time required to be at a medical facility for treatment compared to a subject administered an alternative treatment; (x) a reduction in the cost associated with treatment compared to a subject administered an alternative treatment; as well as any other improved outcome not expressly recited that would be readily envisaged by one skilled in the art.

In some embodiments, a NOS inhibitor composition, as described herein, may be combined with a conventional therapy, as described herein, to treat, ameliorate, and/or reduce the severity of a gastrointestinal cancer or one or more symptoms thereof. In certain embodiments, the NOS inhibitor composition may be administered concurrently with the conventional therapy. In some embodiments, the NOS inhibitor composition may be administered prior to the conventional therapy. In certain embodiments, the NOS inhibitor composition may be administered subsequently to the conventional therapy. Treatment, amelioration, and/or reduction in the severity of a gastrointestinal cancer or one or more symptoms thereof may be characterized by an improved outcome compared an alternative treatment. As used herein, the term “alternative treatment,” may define one or more of a conventional therapy, as described herein, a NOS inhibitor composition comprising a single chemical NOS inhibitor, as described herein, and a NOS inhibitor composition comprising a single biological NOS inhibitor, as described herein. For purposes of the disclosure, an “improved outcome” may define any one of the following: (i) a larger reduction in tumor volume and/or mass compared an alternative treatment; (ii) an improvement in the expected rate of survival of a subject, at, for example, 1 month, 3 months, 6 months, 9 months, 1 year, 2 years, 3 years, 5 years, 10 years, or more, compared to the expected rate of survival of a subject administered an alternative treatment; (iii) a reduction in the prevalence of side effects resulting from the NOS inhibitor composition and conventional therapy compared to a subject administered an alternative treatment; (iv) a reduction in the severity of side effects resulting from the NOS inhibitor composition and conventional therapy compared to a subject administered an alternative treatment; (v) a reduction in the dosage required to achieve an equivalent reduction in tumor volume compared to a subject administered an alternative treatment; (vi) a reduction in the dosage required to achieve an equivalent expected rate of survival of a subject compared to a subject administered an alternative treatment; (vii) a reduction in the likelihood of a recurrence of the gastrointestinal cancer in a subject compared to a subject administered an alternative treatment; (viii) a reduction in the likelihood of a metastasis occurring in a subject compared to a subject administered an alternative treatment; (ix) a reduction in the time required to be at a medical facility for treatment compared to a subject administered an alternative treatment; (x) a reduction in the cost associated with treatment compared to a subject administered an alternative treatment; as well as any other improved outcome not expressly recited that would be readily envisaged by one skilled in the art.

In some embodiments, a “conventional therapy,” as that term is used with respect to combined administration with a NOS inhibitor composition, may refer to a conventional chemotherapy. For purposes of the disclosure, a “conventional chemotherapy,” as described herein may be a compound selected from the group consisting of abemaciclib, abiraterone acetate, acalabrutinib, adagrasib, afatinib dimaleate, albumin-bound paclitaxel, albumin-bound rapamycin (Sirolimus Protein-Bound Particles), alectinib, alpelisib, amifostine, aminolevulinic acid hydrochloride, anastrozole, apalutamide, aprepitant, arsenic trioxide, asciminib hydrochloride, avapritinib, axitinib, azacytidine, belinostat, belzutifan, bendamustine hydrochloride, bexarotene, bicalutamide, binimetinib, bleomycin, bleomycin sulfate, bortezomib, bosutinib, brigatinib, busulfan, cabazitaxel, cabozantinib-S-malate, capecitabine, capmatinib hydrochloride, carboplatin, carfilzomib, carmustine, cedazuridine, ceritinib, chlorambucil, cisplatin, cladribine, clofarabine, cobimetinib fumarate, copanlisib hydrochloride, crizotinib, cyclophosphamide, cytarabine, dabrafenib mesylate, dacarbazine, dacomitinib, dactinomycin, darolutamide, dasatinib, daunorubicin hydrochloride, decitabine, defibrotide sodium, degarelix, dexamethasone, dexrazoxane hydrochloride, docetaxel, doxorubicin hydrochloride, duvelisib, elacestrant dihydrochloride, eltrombopag olamine, enasidenib mesylate, encorafenib, entrectinib, enzalutamide, epirubicin hydrochloride, erdafitinib, eribulin mesylate, erlotinib hydrochloride, etoposide, etoposide phosphate, everolimus, exemestane, fedratinib hydrochloride, fludarabine phosphate, fluorouracil, flutamide, folinic acid, fostamatinib disodium, fulvestrant, futibatinib, gefitinib, gemcitabine hydrochloride, gilteritinib fumarate, glasdegib maleate, goserelin acetate, granisetron, granisetron hydrochloride, hydroxyurea, ibrutinib, idarubicin hydrochloride, idelalisib, ifosfamide, imatinib mesylate, imiquimod, infigratinib phosphate, iobenguane I-131, irinotecan hydrochloride, ivosidenib, ixabepilone, ixazomib citrate, lanreotide acetate, lapatinib ditosylate, larotrectinib sulfate, lenalidomide, lenvatinib mesylate, letrozole, leucovorin calcium, leuprolide acetate, liposomal cytarabine, liposomal doxorubicin hydrochloride, liposomal irinotecan hydrochloride, lomustine, lorlatinib, lurbinectedin, Lu-177 dotatate, Lu-177 vipivotide tetraxetan, mechlorethamine hydrochloride, megestrol acetate, melphalan, melphalan hydrochloride, mercaptopurine, mesna, methotrexate, methotrexate sodium, methylnaltrexone bromide, midostaurin, mitomycin, mitoxantrone hydrochloride, mobocertinib succinate, nelarabine, neratinib maleate, netupitant hydrochloride, nilutamide, nilotinib, niraparib tosylate monohydrate, olaparib, olutasidenib, omacetaxine mepesuccinate, ondansetron hydrochloride, osimertinib mesylate, oxaliplatin, paclitaxel, pacritinib citrate, palbociclib, palonosetron hydrochloride, pamidronate disodium, pazopanib hydrochloride, pemetrexed disodium, pemigatinib, pexidartinib hydrochloride, pirtobrutinib, plerixafor, pomalidomide, ponatinib hydrochloride, pralatrexate, pralsetinib, prednisone, procarbazine hydrochloride, propranolol hydrochloride, Ra-223 dichloride, raloxifene hydrochloride, regorafenib, relugolix, ribociclib, ripretinib, rolapitant hydrochloride, romidepsin, rucaparib camsylate, ruxolitinib phosphate, selinexor, selpercatinib, selumetinib sulfate, sonidegib, sorafenib tosylate, sotorasib, sunitinib malate, talazoparib tosylate, talc, tamoxifen citrate, tazemetostat hydrobromide, temozolomide, temsirolimus, tepotinib hydrochloride, thalidomide, thioguanine, thiotepa, tipiracil hydrochloride, tivozanib hydrochloride, topotecan hydrochloride, toremifene, trabectedin, trametinib dimethyl sulfoxide, tretinoin, trifluridine hydrochloride, tucatinib, uridine triacetate, valrubicin, vandetanib, vemurafenib, venetoclax, vinblastine sulfate, vincristine sulfate, vinorelbine tartrate, vismodegib, vorinostat, zanubrutinib, zoledronic acid, and any combination thereof. One skilled in the art would immediately envisage an appropriate “conventional chemotherapy” based on the particular gastrointestinal cancer disclosed, as well as characteristics of a gastrointestinal cancer subject, such as, for example, the subject's sex, age, stage of the gastrointestinal cancer, genotype of the gastrointestinal cancer, and the like.

In certain embodiments, a “conventional therapy,” as that term is used with respect to combined administration with a NOS inhibitor composition, may refer to a conventional immunotherapy. For purposes of the disclosure, a “conventional immunotherapy,” as described herein may be an immunotherapy, cell-based therapy, and/or gene-based therapy selected from the group consisting of ado-trastuzumab emtansine, aldesleukin, alemtuzumab, amivantamab-vmjw, asparaginase Erwinia chrysanthemi, asparaginase Erwinia chrysanthemi (recombinant)-rywn, atezolizumab, avelumab, axicabtagene ciloleucel, bevacizumab, blinatumomab, brentuximab vedotin, brexucabtagene autoleucel, calaspargase pegol-mknl, camrelizumab, caplacizumab-yhdp, cemiplimab-rwlc, cetuximab, ciltacabtagene autoleucel, daratumumab, daratumumab and hyaluronidase-fihj, darbepoetin alfa, denileukin diftitox, denosumab, dinutuximab, dostarlimab-gxly, durvalumab, elotuzumab, emapalumab-lzsg, enfortumab vedotin-ejfv, epoetin alfa, fam-trastuzumab deruxtecan-nxki, filgrastim, gemtuzumab ozogamicin, glucarpidase, ibritumomab tiuxetan, idecabtagene vicleucel, inotuzumab ozogamicin, ipilimumab, isatuximab-irfc, lisocabtagene maraleucel, loncastuximab tesirine-lpyl, luspatercept-aamt, margetuximab-cmkb, mirvetuximab soravtansine-gynx, mogamulizumab-kpkc, mosunetuzumab-axgb, moxetumomab pasudotox-tdfk, nadofaragene firadenovec-vncg, naxitamab-gqgk, necitumumab, nivolumab, nivolumab and relatlimab-rmbw, obinutuzumab, ofatumumab, palifermin, panitumumab, pegaspargase, pegfilgrastim, pembrolizumab, pertuzumab, pertuzumab, trastuzumab, and hyaluronidase-zzxf, polatuzumab vedotin-piiq, ramucirumab, rasburicase, ravulizumab-cwvz, recombinant HPV bivalent vaccine, recombinant HPV nonavalent vaccine, recombinant HPV quadrivalent vaccine, recombinant interferon alfa-2b, rituximab, rituximab and hyaluronidase human, romiplostim, ropeginterferon alfa-2b-njft, sacituzumab govitecan-hziy, siltuximab, sintilimab, sipuleucel-T, tafasitamab-cxix, tagraxofusp-erzs, talimogene laherparepvec, tebentafusp-tebn, teclistamab-cqyv, tisagenlecleucel, tislelizumab, tisotumab vedotin-tftv, tocilizumab, toripalimab, trastuzumab, trastuzumab and hyaluronidase-oysk, tremelimumab-actl, ziv-aflibercept, and any combination thereof. One skilled in the art would immediately envisage an appropriate “conventional immunotherapy” based on the particular gastrointestinal cancer disclosed, as well as characteristics of a gastrointestinal cancer subject, such as, for example, the subject's sex, age, stage of the gastrointestinal cancer, genotype of the gastrointestinal cancer, and the like.

In certain embodiments, a NOS inhibitor composition, as described herein, may be administered as a suitable dosage form, as described herein, prior to, or alternatively, following a surgery to remove or resect a gastrointestinal cancer.

In some embodiments, a NOS inhibitor composition, as described herein, may be administered as a suitable dosage form, as described herein, to restore and/or maintain healthy and/or optimal expression levels of one or more antigens, as described herein, such as, for example, CD15, CD44, CD70, CD133, as well as any CD antigen that has an abnormal expression in a gastrointestinal cancer disclosed herein that would be readily envisaged by one skilled in the art, in a subject. In certain embodiments, the subject may have a gastrointestinal cancer or one or more symptoms thereof. As used herein, the terms “healthy” and “optimal” refer to expression levels of one or more antigens that would be typical of an equivalent subject that does not have gastrointestinal cancer.

In certain embodiments, a NOS inhibitor composition, as described herein, may be administered as a suitable dosage form, as described herein, to restore and/or maintain healthy and/or optimal expression levels of one or more proteins, as described herein, such as, for example, ALDH1A3, mTOR, p-mTOR, TSC-2, AKT, pAKT, RPS6, p-RPS6, Syp, VEGF, PIGF, as well as any protein or enzyme that has an abnormal expression in a gastrointestinal cancer disclosed herein that would be readily envisaged by one skilled in the art, in a subject. In certain embodiments, the subject may have a gastrointestinal cancer or one or more symptoms thereof. As used herein, the terms “healthy” and “optimal” refer to expression levels of one or more proteins that would be typical of an equivalent subject that does not have gastrointestinal cancer.

In some embodiments, a NOS inhibitor composition, as described herein, may be administered as a suitable dosage form, as described herein, to restore and/or maintain healthy and/or optimal expression levels of one or more NOS isoforms, as described herein, such as, for example, nNOS, iNOS, and eNOS, in a subject. In certain embodiments, the subject may have a gastrointestinal cancer or one or more symptoms thereof. As used herein, the terms “healthy” and “optimal” refer to expression levels of one or more NOS isoforms that would be typical of an equivalent subject that does not have gastrointestinal cancer.

In certain embodiments, a NOS inhibitor composition, as described herein, may be administered as a suitable dosage form, as described herein, to restore and/or maintain healthy and/or optimal levels of one or more NO-indicators, as described herein, such as, for example, 3-Ntyr, NADPH-diaphorase, total nitrite concentration, total nitrate concentration, total nitrite and nitrate concentration, as well as any NO-indicator that has an elevated level in a gastrointestinal cancer disclosed herein that would be readily envisaged by one skilled in the art, in a subject. In certain embodiments, the subject may have a gastrointestinal cancer or one or more symptoms thereof. As used herein, the terms “healthy” and “optimal” refer to levels of one or more NO-indicators that would be typical of an equivalent subject that does not have gastrointestinal cancer.

In certain embodiments, a NOS inhibitor composition, as described herein, may be administered as a suitable dosage form, as described herein, to treat, ameliorate, and/or reduce the severity of an intestinal adenoma or one or more symptoms thereof in a subject (“intestinal adenoma subject”). As used herein, the term “intestinal adenoma” refers to an adenoma that occurs and/or originates in a portion of an intestine including, but not limited to the colon, such as the ascending colon, cecum, descending colon, rectum, sigmoid colon, transverse colon, etc., and the small intestine, such as the duodenum, ileum, jejunum, etc.

In some embodiments, a NOS inhibitor composition comprising 7-NI and/or a salt thereof may be administered as a suitable dosage form, as described herein, to treat, ameliorate, and/or reduce the severity of intestinal adenoma or one or more symptoms thereof in an intestinal adenoma subject. In certain embodiments, an nNOS inhibitor composition comprising 7-NI and/or a salt thereof may be administered to an intestinal adenoma subject to treat, ameliorate, and/or reduce the severity of intestinal adenoma or one or more symptoms thereof in the intestinal adenoma subject: (i) via a route selected from the group consisting of buccally, inhalation, intraarterially, intraarticularly, intracardially, intracerebroventricularly, intradermally, intramedullary, intramuscularly, intraperitoneally, intrathecally, intravenously, intraventricularly, nasally, ophthalmically, orally, rectally, sublabially, sublingually, subcutaneously, topically, and transdermally; and/or (ii) with a frequency selected from the group consisting of four times daily, three times daily, twice daily, once daily, once every two days, once every three days, once every four days, once every five days, once every six days, once a week, once every eight days, once every nine days, once every ten days, once every two weeks, once every three weeks, and once a month; and/or (iii) at a dosage (based on subject weight) selected from the group consisting of 0.1 μg/kg, 0.5 μg/kg, 1 μg/kg, 1.5 μg/kg, 2.0 μg/kg, 2.5 μg/kg, 3.0 μg/kg, 3.5 μg/kg, 4.0 μg/kg, 4.5 μg/kg, 5.0 μg/kg, 10 μg/kg, 15 μg/kg, 20 μg/kg, 25 μg/kg, 30 μg/kg, 35 μg/kg, 40 μg/kg, 45 μg/kg, 50 μg/kg, 55 μg/kg, 60 μg/kg, 65 μg/kg, 70 μg/kg, 75 μg/kg, 80 μg/kg, 85 μg/kg, 90 μg/kg, 95 μg/kg, 100 μg/kg, 150 μg/kg, 200 μg/kg, 250 μg/kg, 300 μg/kg, 350 μg/kg, 400 μg/kg, 450 μg/kg, 500 μg/kg, 550 μg/kg, 600 μg/kg, 650 μg/kg, 700 μg/kg, 750 μg/kg, 800 μg/kg, 850 μg/kg, 900 μg/kg, 1 mg/kg, 1.5 mg/kg, 2.0 mg/kg, 2.5 mg/kg, 3 mg/kg, 3.5 mg/kg, 4.0 mg/kg, 4.5 mg/kg, 5 mg/kg, 5.5 mg/kg, 6 mg/kg, 6.5 mg/kg, 7 mg/kg, 7.5 mg/kg, 8 mg/kg, 8.5 mg/kg, 9 mg/kg, 9.5 mg/kg, 10 mg/kg 10.5 mg/kg, 11 mg/kg, 11.5 mg/kg, 12 mg/kg, 12.5 mg/kg, 13 mg/kg, 13.5 mg/kg, 14 mg/kg, 14.5 mg/kg, 15 mg/kg, 16 mg/kg, 17 mg/kg, 18 mg/kg, 19 mg/kg, 20 mg/kg, 21 mg/kg, 22 mg/kg, 23 mg/kg, 24 mg/kg, 25 mg/kg, 30 mg/kg, 35 mg/kg, 40 mg/kg, 45 mg/kg, 50 mg/kg, 55 mg/kg, 60 mg/kg, 65 mg/kg, 70 mg/kg, 75 mg/kg, 80 mg/kg, 85 mg/kg, 90 mg/kg, 95 mg/kg, 100 mg/kg, 105 mg/kg, 110 mg/kg, 115 mg/kg, 120 mg/kg, 125 mg/kg, 130 mg/kg, 135 mg/kg, 140 mg/kg, 145 mg/kg, 150 mg/kg, 155 mg/kg, 160 mg/kg, 165 mg/kg, 170 mg/kg, 175 mg/kg, 180 mg/kg, 185 mg/kg, 190 mg/kg, 195 mg/kg, 200 mg/kg, and any fraction or integer in between any two of the preceding amounts.

In certain embodiments, a NOS inhibitor composition comprising 3-Br-7-NI and/or a salt thereof may be administered as a suitable dosage form, as described herein, to treat, ameliorate, and/or reduce the severity of intestinal adenoma or one or more symptoms thereof in an intestinal adenoma subject. In some embodiments, an nNOS inhibitor composition comprising 3-Br-7-NI and/or a salt thereof may be administered to an intestinal adenoma subject to treat, ameliorate, and/or reduce the severity of intestinal adenoma or one or more symptoms thereof in the intestinal adenoma subject: (i) via a route selected from the group consisting of buccally, inhalation, intraarterially, intraarticularly, intracardially, intracerebroventricularly, intradermally, intramedullary, intramuscularly, intraperitoneally, intrathecally, intravenously, intraventricularly, nasally, ophthalmically, orally, rectally, sublabially, sublingually, subcutaneously, topically, and transdermally; and/or (ii) with a frequency selected from the group consisting of four times daily, three times daily, twice daily, once daily, once every two days, once every three days, once every four days, once every five days, once every six days, once a week, once every eight days, once every nine days, once every ten days, once every two weeks, once every three weeks, and once a month; and/or (iii) at a dosage (based on subject weight) selected from the group consisting of 0.1 μg/kg, 0.5 μg/kg, 1 μg/kg, 1.5 μg/kg, 2.0 μg/kg, 2.5 μg/kg, 3.0 μg/kg, 3.5 μg/kg, 4.0 μg/kg, 4.5 μg/kg, 5.0 μg/kg, 10 μg/kg, 15 μg/kg, 20 μg/kg, 25 μg/kg, 30 μg/kg, 35 μg/kg, 40 μg/kg, 45 μg/kg, 50 μg/kg, 55 μg/kg, 60 μg/kg, 65 μg/kg, 70 μg/kg, 75 μg/kg, 80 μg/kg, 85 μg/kg, 90 μg/kg, 95 μg/kg, 100 μg/kg, 150 μg/kg, 200 μg/kg, 250 μg/kg, 300 μg/kg, 350 μg/kg, 400 μg/kg, 450 μg/kg, 500 μg/kg, 550 μg/kg, 600 μg/kg, 650 μg/kg, 700 μg/kg, 750 μg/kg, 800 μg/kg, 850 μg/kg, 900 μg/kg, 1 mg/kg, 1.5 mg/kg, 2.0 mg/kg, 2.5 mg/kg, 3 mg/kg, 3.5 mg/kg, 4.0 mg/kg, 4.5 mg/kg, 5 mg/kg, 5.5 mg/kg, 6 mg/kg, 6.5 mg/kg, 7 mg/kg, 7.5 mg/kg, 8 mg/kg, 8.5 mg/kg, 9 mg/kg, 9.5 mg/kg, 10 mg/kg 10.5 mg/kg, 11 mg/kg, 11.5 mg/kg, 12 mg/kg, 12.5 mg/kg, 13 mg/kg, 13.5 mg/kg, 14 mg/kg, 14.5 mg/kg, 15 mg/kg, 16 mg/kg, 17 mg/kg, 18 mg/kg, 19 mg/kg, 20 mg/kg, 21 mg/kg, 22 mg/kg, 23 mg/kg, 24 mg/kg, 25 mg/kg, 30 mg/kg, 35 mg/kg, 40 mg/kg, 45 mg/kg, 50 mg/kg, 55 mg/kg, 60 mg/kg, 65 mg/kg, 70 mg/kg, 75 mg/kg, 80 mg/kg, 85 mg/kg, 90 mg/kg, 95 mg/kg, 100 mg/kg, 105 mg/kg, 110 mg/kg, 115 mg/kg, 120 mg/kg, 125 mg/kg, 130 mg/kg, 135 mg/kg, 140 mg/kg, 145 mg/kg, 150 mg/kg, 155 mg/kg, 160 mg/kg, 165 mg/kg, 170 mg/kg, 175 mg/kg, 180 mg/kg, 185 mg/kg, 190 mg/kg, 195 mg/kg, 200 mg/kg, and any fraction or integer in between any two of the preceding amounts.

In some embodiments, a NOS inhibitor composition comprising L-NPA and/or a salt thereof, such as, for example, L-NPA hydrochloride, may be administered as a suitable dosage form, as described herein, to treat, ameliorate, and/or reduce the severity of intestinal adenoma or one or more symptoms thereof in an intestinal adenoma subject. In certain embodiments, an nNOS inhibitor composition comprising L-NPA and/or a salt thereof, such as, for example, L-NPA hydrochloride may be administered to an intestinal adenoma subject to treat, ameliorate, and/or reduce the severity of intestinal adenoma or one or more symptoms thereof in the intestinal adenoma subject: (i) via a route selected from the group consisting of buccally, inhalation, intraarterially, intraarticularly, intracardially, intracerebroventricularly, intradermally, intramedullary, intramuscularly, intraperitoneally, intrathecally, intravenously, intraventricularly, nasally, ophthalmically, orally, rectally, sublabially, sublingually, subcutaneously, topically, and transdermally; and/or (ii) with a frequency selected from the group consisting of four times daily, three times daily, twice daily, once daily, once every two days, once every three days, once every four days, once every five days, once every six days, once a week, once every eight days, once every nine days, once every ten days, once every two weeks, once every three weeks, and once a month; and/or (iii) at a dosage (based on subject weight) selected from the group consisting of 0.1 μg/kg, 0.5 μg/kg, 1 μg/kg, 1.5 μg/kg, 2.0 μg/kg, 2.5 μg/kg, 3.0 μg/kg, 3.5 μg/kg, 4.0 μg/kg, 4.5 μg/kg, 5.0 μg/kg, 10 μg/kg, 15 μg/kg, 20 μg/kg, 25 μg/kg, 30 μg/kg, 35 μg/kg, 40 μg/kg, 45 μg/kg, 50 μg/kg, 55 μg/kg, 60 μg/kg, 65 μg/kg, 70 μg/kg, 75 μg/kg, 80 μg/kg, 85 μg/kg, 90 μg/kg, 95 μg/kg, 100 μg/kg, 150 μg/kg, 200 μg/kg, 250 μg/kg, 300 μg/kg, 350 μg/kg, 400 μg/kg, 450 μg/kg, 500 μg/kg, 550 μg/kg, 600 μg/kg, 650 μg/kg, 700 μg/kg, 750 μg/kg, 800 μg/kg, 850 μg/kg, 900 μg/kg, 1 mg/kg, 1.5 mg/kg, 2.0 mg/kg, 2.5 mg/kg, 3 mg/kg, 3.5 mg/kg, 4.0 mg/kg, 4.5 mg/kg, 5 mg/kg, 5.5 mg/kg, 6 mg/kg, 6.5 mg/kg, 7 mg/kg, 7.5 mg/kg, 8 mg/kg, 8.5 mg/kg, 9 mg/kg, 9.5 mg/kg, 10 mg/kg 10.5 mg/kg, 11 mg/kg, 11.5 mg/kg, 12 mg/kg, 12.5 mg/kg, 13 mg/kg, 13.5 mg/kg, 14 mg/kg, 14.5 mg/kg, 15 mg/kg, 16 mg/kg, 17 mg/kg, 18 mg/kg, 19 mg/kg, 20 mg/kg, 21 mg/kg, 22 mg/kg, 23 mg/kg, 24 mg/kg, 25 mg/kg, 30 mg/kg, 35 mg/kg, 40 mg/kg, 45 mg/kg, 50 mg/kg, 55 mg/kg, 60 mg/kg, 65 mg/kg, 70 mg/kg, 75 mg/kg, 80 mg/kg, 85 mg/kg, 90 mg/kg, 95 mg/kg, 100 mg/kg, 105 mg/kg, 110 mg/kg, 115 mg/kg, 120 mg/kg, 125 mg/kg, 130 mg/kg, 135 mg/kg, 140 mg/kg, 145 mg/kg, 150 mg/kg, 155 mg/kg, 160 mg/kg, 165 mg/kg, 170 mg/kg, 175 mg/kg, 180 mg/kg, 185 mg/kg, 190 mg/kg, 195 mg/kg, 200 mg/kg, and any fraction or integer in between any two of the preceding amounts.

In certain embodiments, a NOS inhibitor composition comprising L-NIO and/or a salt thereof, such as, for example, L-NIO dihydrochloride, may be administered as a suitable dosage form, as described herein, to treat, ameliorate, and/or reduce the severity of intestinal adenoma or one or more symptoms thereof in an intestinal adenoma subject. In some embodiments, an nNOS inhibitor composition comprising L-NIO and/or a salt thereof, such as, for example, L-NIO dihydrochloride, may be administered to an intestinal adenoma subject to treat, ameliorate, and/or reduce the severity of intestinal adenoma or one or more symptoms thereof in the intestinal adenoma subject: (i) via a route selected from the group consisting of buccally, inhalation, intraarterially, intraarticularly, intracardially, intracerebroventricularly, intradermally, intramedullary, intramuscularly, intraperitoneally, intrathecally, intravenously, intraventricularly, nasally, ophthalmically, orally, rectally, sublabially, sublingually, subcutaneously, topically, and transdermally; and/or (ii) with a frequency selected from the group consisting of four times daily, three times daily, twice daily, once daily, once every two days, once every three days, once every four days, once every five days, once every six days, once a week, once every eight days, once every nine days, once every ten days, once every two weeks, once every three weeks, and once a month; and/or (iii) at a dosage (based on subject weight) selected from the group consisting of 0.1 μg/kg, 0.5 μg/kg, 1 μg/kg, 1.5 μg/kg, 2.0 μg/kg, 2.5 μg/kg, 3.0 μg/kg, 3.5 μg/kg, 4.0 μg/kg, 4.5 μg/kg, 5.0 μg/kg, 10 μg/kg, 15 μg/kg, 20 μg/kg, 25 μg/kg, 30 μg/kg, 35 μg/kg, 40 μg/kg, 45 μg/kg, 50 μg/kg, 55 μg/kg, 60 μg/kg, 65 μg/kg, 70 μg/kg, 75 μg/kg, 80 μg/kg, 85 μg/kg, 90 μg/kg, 95 μg/kg, 100 μg/kg, 150 μg/kg, 200 μg/kg, 250 μg/kg, 300 μg/kg, 350 μg/kg, 400 μg/kg, 450 μg/kg, 500 μg/kg, 550 μg/kg, 600 μg/kg, 650 μg/kg, 700 μg/kg, 750 μg/kg, 800 μg/kg, 850 μg/kg, 900 μg/kg, 1 mg/kg, 1.5 mg/kg, 2.0 mg/kg, 2.5 mg/kg, 3 mg/kg, 3.5 mg/kg, 4.0 mg/kg, 4.5 mg/kg, 5 mg/kg, 5.5 mg/kg, 6 mg/kg, 6.5 mg/kg, 7 mg/kg, 7.5 mg/kg, 8 mg/kg, 8.5 mg/kg, 9 mg/kg, 9.5 mg/kg, 10 mg/kg 10.5 mg/kg, 11 mg/kg, 11.5 mg/kg, 12 mg/kg, 12.5 mg/kg, 13 mg/kg, 13.5 mg/kg, 14 mg/kg, 14.5 mg/kg, 15 mg/kg, 16 mg/kg, 17 mg/kg, 18 mg/kg, 19 mg/kg, 20 mg/kg, 21 mg/kg, 22 mg/kg, 23 mg/kg, 24 mg/kg, 25 mg/kg, 30 mg/kg, 35 mg/kg, 40 mg/kg, 45 mg/kg, 50 mg/kg, 55 mg/kg, 60 mg/kg, 65 mg/kg, 70 mg/kg, 75 mg/kg, 80 mg/kg, 85 mg/kg, 90 mg/kg, 95 mg/kg, 100 mg/kg, 105 mg/kg, 110 mg/kg, 115 mg/kg, 120 mg/kg, 125 mg/kg, 130 mg/kg, 135 mg/kg, 140 mg/kg, 145 mg/kg, 150 mg/kg, 155 mg/kg, 160 mg/kg, 165 mg/kg, 170 mg/kg, 175 mg/kg, 180 mg/kg, 185 mg/kg, 190 mg/kg, 195 mg/kg, 200 mg/kg, and any fraction or integer in between any two of the preceding amounts.

In some embodiments, a NOS inhibitor composition comprising ARL-17477 and/or a salt thereof, such as, for example, ARL-17477 dihydrochloride, may be administered as a suitable dosage form, as described herein, to treat, ameliorate, and/or reduce the severity of intestinal adenoma or one or more symptoms thereof in an intestinal adenoma subject. In certain embodiments, an nNOS inhibitor composition comprising ARL-17477 and/or a salt thereof, such as, for example, ARL-17477 dihydrochloride, may be administered to an intestinal adenoma subject to treat, ameliorate, and/or reduce the severity of intestinal adenoma or one or more symptoms thereof in the intestinal adenoma subject: (i) via a route selected from the group consisting of buccally, inhalation, intraarterially, intraarticularly, intracardially, intracerebroventricularly, intradermally, intramedullary, intramuscularly, intraperitoneally, intrathecally, intravenously, intraventricularly, nasally, ophthalmically, orally, rectally, sublabially, sublingually, subcutaneously, topically, and transdermally; and/or (ii) with a frequency selected from the group consisting of four times daily, three times daily, twice daily, once daily, once every two days, once every three days, once every four days, once every five days, once every six days, once a week, once every eight days, once every nine days, once every ten days, once every two weeks, once every three weeks, and once a month; and/or (iii) at a dosage (based on subject weight) selected from the group consisting of 0.1 μg/kg, 0.5 μg/kg, 1 μg/kg, 1.5 μg/kg, 2.0 μg/kg, 2.5 μg/kg, 3.0 μg/kg, 3.5 μg/kg, 4.0 μg/kg, 4.5 μg/kg, 5.0 μg/kg, 10 μg/kg, 15 μg/kg, 20 μg/kg, 25 μg/kg, 30 μg/kg, 35 μg/kg, 40 μg/kg, 45 μg/kg, 50 μg/kg, 55 μg/kg, 60 μg/kg, 65 μg/kg, 70 μg/kg, 75 μg/kg, 80 μg/kg, 85 μg/kg, 90 μg/kg, 95 μg/kg, 100 μg/kg, 150 μg/kg, 200 μg/kg, 250 μg/kg, 300 μg/kg, 350 μg/kg, 400 μg/kg, 450 μg/kg, 500 μg/kg, 550 μg/kg, 600 μg/kg, 650 μg/kg, 700 μg/kg, 750 μg/kg, 800 μg/kg, 850 μg/kg, 900 μg/kg, 1 mg/kg, 1.5 mg/kg, 2.0 mg/kg, 2.5 mg/kg, 3 mg/kg, 3.5 mg/kg, 4.0 mg/kg, 4.5 mg/kg, 5 mg/kg, 5.5 mg/kg, 6 mg/kg, 6.5 mg/kg, 7 mg/kg, 7.5 mg/kg, 8 mg/kg, 8.5 mg/kg, 9 mg/kg, 9.5 mg/kg, 10 mg/kg 10.5 mg/kg, 11 mg/kg, 11.5 mg/kg, 12 mg/kg, 12.5 mg/kg, 13 mg/kg, 13.5 mg/kg, 14 mg/kg, 14.5 mg/kg, 15 mg/kg, 16 mg/kg, 17 mg/kg, 18 mg/kg, 19 mg/kg, 20 mg/kg, 21 mg/kg, 22 mg/kg, 23 mg/kg, 24 mg/kg, 25 mg/kg, 30 mg/kg, 35 mg/kg, 40 mg/kg, 45 mg/kg, 50 mg/kg, 55 mg/kg, 60 mg/kg, 65 mg/kg, 70 mg/kg, 75 mg/kg, 80 mg/kg, 85 mg/kg, 90 mg/kg, 95 mg/kg, 100 mg/kg, 105 mg/kg, 110 mg/kg, 115 mg/kg, 120 mg/kg, 125 mg/kg, 130 mg/kg, 135 mg/kg, 140 mg/kg, 145 mg/kg, 150 mg/kg, 155 mg/kg, 160 mg/kg, 165 mg/kg, 170 mg/kg, 175 mg/kg, 180 mg/kg, 185 mg/kg, 190 mg/kg, 195 mg/kg, 200 mg/kg, and any fraction or integer in between any two of the preceding amounts.

In certain embodiments, a NOS inhibitor composition comprising L-NNA and/or a salt thereof may be administered as a suitable dosage form, as described herein, to treat, ameliorate, and/or reduce the severity of intestinal adenoma or one or more symptoms thereof in an intestinal adenoma subject. In some embodiments, an nNOS inhibitor composition comprising L-NNA and/or a salt thereof may be administered to an intestinal adenoma subject to treat, ameliorate, and/or reduce the severity of intestinal adenoma or one or more symptoms thereof in the intestinal adenoma subject: (i) via a route selected from the group consisting of buccally, inhalation, intraarterially, intraarticularly, intracardially, intracerebroventricularly, intradermally, intramedullary, intramuscularly, intraperitoneally, intrathecally, intravenously, intraventricularly, nasally, ophthalmically, orally, rectally, sublabially, sublingually, subcutaneously, topically, and transdermally; and/or (ii) with a frequency selected from the group consisting of four times daily, three times daily, twice daily, once daily, once every two days, once every three days, once every four days, once every five days, once every six days, once a week, once every eight days, once every nine days, once every ten days, once every two weeks, once every three weeks, and once a month; and/or (iii) at a dosage (based on subject weight) selected from the group consisting of 0.1 μg/kg, 0.5 μg/kg, 1 μg/kg, 1.5 μg/kg, 2.0 μg/kg, 2.5 μg/kg, 3.0 μg/kg, 3.5 μg/kg, 4.0 μg/kg, 4.5 μg/kg, 5.0 μg/kg, 10 μg/kg, 15 μg/kg, 20 μg/kg, 25 μg/kg, 30 μg/kg, 35 μg/kg, 40 μg/kg, 45 μg/kg, 50 μg/kg, 55 μg/kg, 60 μg/kg, 65 μg/kg, 70 μg/kg, 75 μg/kg, 80 μg/kg, 85 μg/kg, 90 μg/kg, 95 μg/kg, 100 μg/kg, 150 μg/kg, 200 μg/kg, 250 μg/kg, 300 μg/kg, 350 μg/kg, 400 μg/kg, 450 μg/kg, 500 μg/kg, 550 μg/kg, 600 μg/kg, 650 μg/kg, 700 μg/kg, 750 μg/kg, 800 μg/kg, 850 μg/kg, 900 μg/kg, 1 mg/kg, 1.5 mg/kg, 2.0 mg/kg, 2.5 mg/kg, 3 mg/kg, 3.5 mg/kg, 4.0 mg/kg, 4.5 mg/kg, 5 mg/kg, 5.5 mg/kg, 6 mg/kg, 6.5 mg/kg, 7 mg/kg, 7.5 mg/kg, 8 mg/kg, 8.5 mg/kg, 9 mg/kg, 9.5 mg/kg, 10 mg/kg 10.5 mg/kg, 11 mg/kg, 11.5 mg/kg, 12 mg/kg, 12.5 mg/kg, 13 mg/kg, 13.5 mg/kg, 14 mg/kg, 14.5 mg/kg, 15 mg/kg, 16 mg/kg, 17 mg/kg, 18 mg/kg, 19 mg/kg, 20 mg/kg, 21 mg/kg, 22 mg/kg, 23 mg/kg, 24 mg/kg, 25 mg/kg, 30 mg/kg, 35 mg/kg, 40 mg/kg, 45 mg/kg, 50 mg/kg, 55 mg/kg, 60 mg/kg, 65 mg/kg, 70 mg/kg, 75 mg/kg, 80 mg/kg, 85 mg/kg, 90 mg/kg, 95 mg/kg, 100 mg/kg, 105 mg/kg, 110 mg/kg, 115 mg/kg, 120 mg/kg, 125 mg/kg, 130 mg/kg, 135 mg/kg, 140 mg/kg, 145 mg/kg, 150 mg/kg, 155 mg/kg, 160 mg/kg, 165 mg/kg, 170 mg/kg, 175 mg/kg, 180 mg/kg, 185 mg/kg, 190 mg/kg, 195 mg/kg, 200 mg/kg, and any fraction or integer in between any two of the preceding amounts.

In some embodiments, a NOS inhibitor composition comprising L-NAME and/or a salt thereof, such as, for example, L-NAME hydrochloride, may be administered as a suitable dosage form, as described herein, to treat, ameliorate, and/or reduce the severity of intestinal adenoma or one or more symptoms thereof in an intestinal adenoma subject. In certain embodiments, an nNOS inhibitor composition comprising L-NAME and/or a salt thereof, such as, for example, L-NAME hydrochloride, may be administered to an intestinal adenoma subject to treat, ameliorate, and/or reduce the severity of intestinal adenoma or one or more symptoms thereof in the intestinal adenoma subject: (i) via a route selected from the group consisting of buccally, inhalation, intraarterially, intraarticularly, intracardially, intracerebroventricularly, intradermally, intramedullary, intramuscularly, intraperitoneally, intrathecally, intravenously, intraventricularly, nasally, ophthalmically, orally, rectally, sublabially, sublingually, subcutaneously, topically, and transdermally; and/or (ii) with a frequency selected from the group consisting of four times daily, three times daily, twice daily, once daily, once every two days, once every three days, once every four days, once every five days, once every six days, once a week, once every eight days, once every nine days, once every ten days, once every two weeks, once every three weeks, and once a month; and/or (iii) at a dosage (based on subject weight) selected from the group consisting of 0.1 μg/kg, 0.5 μg/kg, 1 μg/kg, 1.5 μg/kg, 2.0 μg/kg, 2.5 μg/kg, 3.0 μg/kg, 3.5 μg/kg, 4.0 μg/kg, 4.5 μg/kg, 5.0 μg/kg, 10 μg/kg, 15 μg/kg, 20 μg/kg, 25 μg/kg, 30 μg/kg, 35 μg/kg, 40 μg/kg, 45 μg/kg, 50 μg/kg, 55 μg/kg, 60 μg/kg, 65 μg/kg, 70 μg/kg, 75 μg/kg, 80 μg/kg, 85 μg/kg, 90 μg/kg, 95 μg/kg, 100 μg/kg, 150 μg/kg, 200 μg/kg, 250 μg/kg, 300 μg/kg, 350 μg/kg, 400 μg/kg, 450 μg/kg, 500 μg/kg, 550 μg/kg, 600 μg/kg, 650 μg/kg, 700 μg/kg, 750 μg/kg, 800 μg/kg, 850 μg/kg, 900 μg/kg, 1 mg/kg, 1.5 mg/kg, 2.0 mg/kg, 2.5 mg/kg, 3 mg/kg, 3.5 mg/kg, 4.0 mg/kg, 4.5 mg/kg, 5 mg/kg, 5.5 mg/kg, 6 mg/kg, 6.5 mg/kg, 7 mg/kg, 7.5 mg/kg, 8 mg/kg, 8.5 mg/kg, 9 mg/kg, 9.5 mg/kg, 10 mg/kg 10.5 mg/kg, 11 mg/kg, 11.5 mg/kg, 12 mg/kg, 12.5 mg/kg, 13 mg/kg, 13.5 mg/kg, 14 mg/kg, 14.5 mg/kg, 15 mg/kg, 16 mg/kg, 17 mg/kg, 18 mg/kg, 19 mg/kg, 20 mg/kg, 21 mg/kg, 22 mg/kg, 23 mg/kg, 24 mg/kg, 25 mg/kg, 30 mg/kg, 35 mg/kg, 40 mg/kg, 45 mg/kg, 50 mg/kg, 55 mg/kg, 60 mg/kg, 65 mg/kg, 70 mg/kg, 75 mg/kg, 80 mg/kg, 85 mg/kg, 90 mg/kg, 95 mg/kg, 100 mg/kg, 105 mg/kg, 110 mg/kg, 115 mg/kg, 120 mg/kg, 125 mg/kg, 130 mg/kg, 135 mg/kg, 140 mg/kg, 145 mg/kg, 150 mg/kg, 155 mg/kg, 160 mg/kg, 165 mg/kg, 170 mg/kg, 175 mg/kg, 180 mg/kg, 185 mg/kg, 190 mg/kg, 195 mg/kg, 200 mg/kg, and any fraction or integer in between any two of the preceding amounts.

In certain embodiments, a NOS inhibitor composition comprising L-NMMA and/or a salt thereof, such as, for example, L-NMMA acetate, may be administered as a suitable dosage form, as described herein, to treat, ameliorate, and/or reduce the severity of intestinal adenoma or one or more symptoms thereof in an intestinal adenoma subject. In some embodiments, an nNOS inhibitor composition comprising L-NMMA and/or a salt thereof, such as, for example, L-NMMA acetate, may be administered to an intestinal adenoma subject to treat, ameliorate, and/or reduce the severity of intestinal adenoma or one or more symptoms thereof in the intestinal adenoma subject: (i) via a route selected from the group consisting of buccally, inhalation, intraarterially, intraarticularly, intracardially, intracerebroventricularly, intradermally, intramedullary, intramuscularly, intraperitoneally, intrathecally, intravenously, intraventricularly, nasally, ophthalmically, orally, rectally, sublabially, sublingually, subcutaneously, topically, and transdermally; and/or (ii) with a frequency selected from the group consisting of four times daily, three times daily, twice daily, once daily, once every two days, once every three days, once every four days, once every five days, once every six days, once a week, once every eight days, once every nine days, once every ten days, once every two weeks, once every three weeks, and once a month; and/or (iii) at a dosage (based on subject weight) selected from the group consisting of 0.1 μg/kg, 0.5 μg/kg, 1 μg/kg, 1.5 μg/kg, 2.0 μg/kg, 2.5 μg/kg, 3.0 μg/kg, 3.5 μg/kg, 4.0 μg/kg, 4.5 μg/kg, 5.0 μg/kg, 10 μg/kg, 15 μg/kg, 20 μg/kg, 25 μg/kg, 30 μg/kg, 35 μg/kg, 40 μg/kg, 45 μg/kg, 50 μg/kg, 55 μg/kg, 60 μg/kg, 65 μg/kg, 70 μg/kg, 75 μg/kg, 80 μg/kg, 85 μg/kg, 90 μg/kg, 95 μg/kg, 100 μg/kg, 150 μg/kg, 200 μg/kg, 250 μg/kg, 300 μg/kg, 350 μg/kg, 400 μg/kg, 450 μg/kg, 500 μg/kg, 550 μg/kg, 600 μg/kg, 650 μg/kg, 700 μg/kg, 750 μg/kg, 800 μg/kg, 850 μg/kg, 900 μg/kg, 1 mg/kg, 1.5 mg/kg, 2.0 mg/kg, 2.5 mg/kg, 3 mg/kg, 3.5 mg/kg, 4.0 mg/kg, 4.5 mg/kg, 5 mg/kg, 5.5 mg/kg, 6 mg/kg, 6.5 mg/kg, 7 mg/kg, 7.5 mg/kg, 8 mg/kg, 8.5 mg/kg, 9 mg/kg, 9.5 mg/kg, 10 mg/kg 10.5 mg/kg, 11 mg/kg, 11.5 mg/kg, 12 mg/kg, 12.5 mg/kg, 13 mg/kg, 13.5 mg/kg, 14 mg/kg, 14.5 mg/kg, 15 mg/kg, 16 mg/kg, 17 mg/kg, 18 mg/kg, 19 mg/kg, 20 mg/kg, 21 mg/kg, 22 mg/kg, 23 mg/kg, 24 mg/kg, 25 mg/kg, 30 mg/kg, 35 mg/kg, 40 mg/kg, 45 mg/kg, 50 mg/kg, 55 mg/kg, 60 mg/kg, 65 mg/kg, 70 mg/kg, 75 mg/kg, 80 mg/kg, 85 mg/kg, 90 mg/kg, 95 mg/kg, 100 mg/kg, 105 mg/kg, 110 mg/kg, 115 mg/kg, 120 mg/kg, 125 mg/kg, 130 mg/kg, 135 mg/kg, 140 mg/kg, 145 mg/kg, 150 mg/kg, 155 mg/kg, 160 mg/kg, 165 mg/kg, 170 mg/kg, 175 mg/kg, 180 mg/kg, 185 mg/kg, 190 mg/kg, 195 mg/kg, 200 mg/kg, and any fraction or integer in between any two of the preceding amounts.

In some embodiments, a NOS inhibitor composition comprising GW-274150 and/or a salt thereof, such as, for example, GW-274150 phosphate, may be administered as a suitable dosage form, as described herein, to treat, ameliorate, and/or reduce the severity of intestinal adenoma or one or more symptoms thereof in an intestinal adenoma subject. In certain embodiments, an nNOS inhibitor composition comprising GW-274150 and/or a salt thereof, such as, for example, GW-274150 phosphate, may be administered to an intestinal adenoma subject to treat, ameliorate, and/or reduce the severity of intestinal adenoma or one or more symptoms thereof in the intestinal adenoma subject: (i) via a route selected from the group consisting of buccally, inhalation, intraarterially, intraarticularly, intracardially, intracerebroventricularly, intradermally, intramedullary, intramuscularly, intraperitoneally, intrathecally, intravenously, intraventricularly, nasally, ophthalmically, orally, rectally, sublabially, sublingually, subcutaneously, topically, and transdermally; and/or (ii) with a frequency selected from the group consisting of four times daily, three times daily, twice daily, once daily, once every two days, once every three days, once every four days, once every five days, once every six days, once a week, once every eight days, once every nine days, once every ten days, once every two weeks, once every three weeks, and once a month; and/or (iii) at a dosage (based on subject weight) selected from the group consisting of 0.1 μg/kg, 0.5 μg/kg, 1 μg/kg, 1.5 μg/kg, 2.0 μg/kg, 2.5 μg/kg, 3.0 μg/kg, 3.5 μg/kg, 4.0 μg/kg, 4.5 μg/kg, 5.0 μg/kg, 10 μg/kg, 15 μg/kg, 20 μg/kg, 25 μg/kg, 30 μg/kg, 35 μg/kg, 40 μg/kg, 45 μg/kg, 50 μg/kg, 55 μg/kg, 60 μg/kg, 65 μg/kg, 70 μg/kg, 75 μg/kg, 80 μg/kg, 85 μg/kg, 90 μg/kg, 95 μg/kg, 100 μg/kg, 150 μg/kg, 200 μg/kg, 250 μg/kg, 300 μg/kg, 350 μg/kg, 400 μg/kg, 450 μg/kg, 500 μg/kg, 550 μg/kg, 600 μg/kg, 650 μg/kg, 700 μg/kg, 750 μg/kg, 800 μg/kg, 850 μg/kg, 900 μg/kg, 1 mg/kg, 1.5 mg/kg, 2.0 mg/kg, 2.5 mg/kg, 3 mg/kg, 3.5 mg/kg, 4.0 mg/kg, 4.5 mg/kg, 5 mg/kg, 5.5 mg/kg, 6 mg/kg, 6.5 mg/kg, 7 mg/kg, 7.5 mg/kg, 8 mg/kg, 8.5 mg/kg, 9 mg/kg, 9.5 mg/kg, 10 mg/kg 10.5 mg/kg, 11 mg/kg, 11.5 mg/kg, 12 mg/kg, 12.5 mg/kg, 13 mg/kg, 13.5 mg/kg, 14 mg/kg, 14.5 mg/kg, 15 mg/kg, 16 mg/kg, 17 mg/kg, 18 mg/kg, 19 mg/kg, 20 mg/kg, 21 mg/kg, 22 mg/kg, 23 mg/kg, 24 mg/kg, 25 mg/kg, 30 mg/kg, 35 mg/kg, 40 mg/kg, 45 mg/kg, 50 mg/kg, 55 mg/kg, 60 mg/kg, 65 mg/kg, 70 mg/kg, 75 mg/kg, 80 mg/kg, 85 mg/kg, 90 mg/kg, 95 mg/kg, 100 mg/kg, 105 mg/kg, 110 mg/kg, 115 mg/kg, 120 mg/kg, 125 mg/kg, 130 mg/kg, 135 mg/kg, 140 mg/kg, 145 mg/kg, 150 mg/kg, 155 mg/kg, 160 mg/kg, 165 mg/kg, 170 mg/kg, 175 mg/kg, 180 mg/kg, 185 mg/kg, 190 mg/kg, 195 mg/kg, 200 mg/kg, and any fraction or integer in between any two of the preceding amounts.

In certain embodiments, a NOS inhibitor composition comprising L-NIL and/or a salt thereof, such as, for example, L-NIL hydrochloride, may be administered as a suitable dosage form, as described herein, to treat, ameliorate, and/or reduce the severity of intestinal adenoma or one or more symptoms thereof in an intestinal adenoma subject. In some embodiments, an nNOS inhibitor composition comprising L-NIL and/or a salt thereof, such as, for example, L-NIL hydrochloride, may be administered to an intestinal adenoma subject to treat, ameliorate, and/or reduce the severity of intestinal adenoma or one or more symptoms thereof in the intestinal adenoma subject: (i) via a route selected from the group consisting of buccally, inhalation, intraarterially, intraarticularly, intracardially, intracerebroventricularly, intradermally, intramedullary, intramuscularly, intraperitoneally, intrathecally, intravenously, intraventricularly, nasally, ophthalmically, orally, rectally, sublabially, sublingually, subcutaneously, topically, and transdermally; and/or (ii) with a frequency selected from the group consisting of four times daily, three times daily, twice daily, once daily, once every two days, once every three days, once every four days, once every five days, once every six days, once a week, once every eight days, once every nine days, once every ten days, once every two weeks, once every three weeks, and once a month; and/or (iii) at a dosage (based on subject weight) selected from the group consisting of 0.1 μg/kg, 0.5 μg/kg, 1 μg/kg, 1.5 μg/kg, 2.0 μg/kg, 2.5 μg/kg, 3.0 μg/kg, 3.5 μg/kg, 4.0 μg/kg, 4.5 μg/kg, 5.0 μg/kg, 10 μg/kg, 15 μg/kg, 20 μg/kg, 25 μg/kg, 30 μg/kg, 35 μg/kg, 40 μg/kg, 45 μg/kg, 50 μg/kg, 55 μg/kg, 60 μg/kg, 65 μg/kg, 70 μg/kg, 75 μg/kg, 80 μg/kg, 85 μg/kg, 90 μg/kg, 95 μg/kg, 100 μg/kg, 150 μg/kg, 200 μg/kg, 250 μg/kg, 300 μg/kg, 350 μg/kg, 400 μg/kg, 450 μg/kg, 500 μg/kg, 550 μg/kg, 600 μg/kg, 650 μg/kg, 700 μg/kg, 750 μg/kg, 800 μg/kg, 850 μg/kg, 900 μg/kg, 1 mg/kg, 1.5 mg/kg, 2.0 mg/kg, 2.5 mg/kg, 3 mg/kg, 3.5 mg/kg, 4.0 mg/kg, 4.5 mg/kg, 5 mg/kg, 5.5 mg/kg, 6 mg/kg, 6.5 mg/kg, 7 mg/kg, 7.5 mg/kg, 8 mg/kg, 8.5 mg/kg, 9 mg/kg, 9.5 mg/kg, 10 mg/kg 10.5 mg/kg, 11 mg/kg, 11.5 mg/kg, 12 mg/kg, 12.5 mg/kg, 13 mg/kg, 13.5 mg/kg, 14 mg/kg, 14.5 mg/kg, 15 mg/kg, 16 mg/kg, 17 mg/kg, 18 mg/kg, 19 mg/kg, 20 mg/kg, 21 mg/kg, 22 mg/kg, 23 mg/kg, 24 mg/kg, 25 mg/kg, 30 mg/kg, 35 mg/kg, 40 mg/kg, 45 mg/kg, 50 mg/kg, 55 mg/kg, 60 mg/kg, 65 mg/kg, 70 mg/kg, 75 mg/kg, 80 mg/kg, 85 mg/kg, 90 mg/kg, 95 mg/kg, 100 mg/kg, 105 mg/kg, 110 mg/kg, 115 mg/kg, 120 mg/kg, 125 mg/kg, 130 mg/kg, 135 mg/kg, 140 mg/kg, 145 mg/kg, 150 mg/kg, 155 mg/kg, 160 mg/kg, 165 mg/kg, 170 mg/kg, 175 mg/kg, 180 mg/kg, 185 mg/kg, 190 mg/kg, 195 mg/kg, 200 mg/kg, and any fraction or integer in between any two of the preceding amounts.

In some embodiments, a NOS inhibitor composition comprising 1400W and/or a salt thereof, such as, for example, 1400W dihydrochloride, may be administered as a suitable dosage form, as described herein, to treat, ameliorate, and/or reduce the severity of intestinal adenoma or one or more symptoms thereof in an intestinal adenoma subject. In certain embodiments, an nNOS inhibitor composition comprising 1400W and/or a salt thereof, such as, for example, 1400W dihydrochloride, may be administered to an intestinal adenoma subject to treat, ameliorate, and/or reduce the severity of intestinal adenoma or one or more symptoms thereof in the intestinal adenoma subject: (i) via a route selected from the group consisting of buccally, inhalation, intraarterially, intraarticularly, intracardially, intracerebroventricularly, intradermally, intramedullary, intramuscularly, intraperitoneally, intrathecally, intravenously, intraventricularly, nasally, ophthalmically, orally, rectally, sublabially, sublingually, subcutaneously, topically, and transdermally; and/or (ii) with a frequency selected from the group consisting of four times daily, three times daily, twice daily, once daily, once every two days, once every three days, once every four days, once every five days, once every six days, once a week, once every eight days, once every nine days, once every ten days, once every two weeks, once every three weeks, and once a month; and/or (iii) at a dosage (based on subject weight) selected from the group consisting of 0.1 μg/kg, 0.5 μg/kg, 1 μg/kg, 1.5 μg/kg, 2.0 μg/kg, 2.5 μg/kg, 3.0 μg/kg, 3.5 μg/kg, 4.0 μg/kg, 4.5 μg/kg, 5.0 μg/kg, 10 μg/kg, 15 μg/kg, 20 μg/kg, 25 μg/kg, 30 μg/kg, 35 μg/kg, 40 μg/kg, 45 μg/kg, 50 μg/kg, 55 μg/kg, 60 μg/kg, 65 μg/kg, 70 μg/kg, 75 μg/kg, 80 μg/kg, 85 μg/kg, 90 μg/kg, 95 μg/kg, 100 μg/kg, 150 μg/kg, 200 μg/kg, 250 μg/kg, 300 μg/kg, 350 μg/kg, 400 μg/kg, 450 μg/kg, 500 μg/kg, 550 μg/kg, 600 μg/kg, 650 μg/kg, 700 μg/kg, 750 μg/kg, 800 μg/kg, 850 μg/kg, 900 μg/kg, 1 mg/kg, 1.5 mg/kg, 2.0 mg/kg, 2.5 mg/kg, 3 mg/kg, 3.5 mg/kg, 4.0 mg/kg, 4.5 mg/kg, 5 mg/kg, 5.5 mg/kg, 6 mg/kg, 6.5 mg/kg, 7 mg/kg, 7.5 mg/kg, 8 mg/kg, 8.5 mg/kg, 9 mg/kg, 9.5 mg/kg, 10 mg/kg 10.5 mg/kg, 11 mg/kg, 11.5 mg/kg, 12 mg/kg, 12.5 mg/kg, 13 mg/kg, 13.5 mg/kg, 14 mg/kg, 14.5 mg/kg, 15 mg/kg, 16 mg/kg, 17 mg/kg, 18 mg/kg, 19 mg/kg, 20 mg/kg, 21 mg/kg, 22 mg/kg, 23 mg/kg, 24 mg/kg, 25 mg/kg, 30 mg/kg, 35 mg/kg, 40 mg/kg, 45 mg/kg, 50 mg/kg, 55 mg/kg, 60 mg/kg, 65 mg/kg, 70 mg/kg, 75 mg/kg, 80 mg/kg, 85 mg/kg, 90 mg/kg, 95 mg/kg, 100 mg/kg, 105 mg/kg, 110 mg/kg, 115 mg/kg, 120 mg/kg, 125 mg/kg, 130 mg/kg, 135 mg/kg, 140 mg/kg, 145 mg/kg, 150 mg/kg, 155 mg/kg, 160 mg/kg, 165 mg/kg, 170 mg/kg, 175 mg/kg, 180 mg/kg, 185 mg/kg, 190 mg/kg, 195 mg/kg, 200 mg/kg, and any fraction or integer in between any two of the preceding amounts.

In certain embodiments, a NOS inhibitor composition comprising OKN-007 and/or a salt thereof, such as, for example, OKN-007 sodium, may be administered as a suitable dosage form, as described herein, to treat, ameliorate, and/or reduce the severity of intestinal adenoma or one or more symptoms thereof in an intestinal adenoma subject. In some embodiments, an nNOS inhibitor composition comprising OKN-007 and/or a salt thereof, such as, for example, OKN-007 sodium, may be administered to an intestinal adenoma subject to treat, ameliorate, and/or reduce the severity of intestinal adenoma or one or more symptoms thereof in the intestinal adenoma subject: (i) via a route selected from the group consisting of buccally, inhalation, intraarterially, intraarticularly, intracardially, intracerebroventricularly, intradermally, intramedullary, intramuscularly, intraperitoneally, intrathecally, intravenously, intraventricularly, nasally, ophthalmically, orally, rectally, sublabially, sublingually, subcutaneously, topically, and transdermally; and/or (ii) with a frequency selected from the group consisting of four times daily, three times daily, twice daily, once daily, once every two days, once every three days, once every four days, once every five days, once every six days, once a week, once every eight days, once every nine days, once every ten days, once every two weeks, once every three weeks, and once a month; and/or (iii) at a dosage (based on subject weight) selected from the group consisting of 0.1 μg/kg, 0.5 μg/kg, 1 μg/kg, 1.5 μg/kg, 2.0 μg/kg, 2.5 μg/kg, 3.0 μg/kg, 3.5 μg/kg, 4.0 μg/kg, 4.5 μg/kg, 5.0 μg/kg, 10 μg/kg, 15 μg/kg, 20 μg/kg, 25 μg/kg, 30 μg/kg, 35 μg/kg, 40 μg/kg, 45 μg/kg, 50 μg/kg, 55 μg/kg, 60 μg/kg, 65 μg/kg, 70 μg/kg, 75 μg/kg, 80 μg/kg, 85 μg/kg, 90 μg/kg, 95 μg/kg, 100 μg/kg, 150 μg/kg, 200 μg/kg, 250 μg/kg, 300 μg/kg, 350 μg/kg, 400 μg/kg, 450 μg/kg, 500 μg/kg, 550 μg/kg, 600 μg/kg, 650 μg/kg, 700 μg/kg, 750 μg/kg, 800 μg/kg, 850 μg/kg, 900 μg/kg, 1 mg/kg, 1.5 mg/kg, 2.0 mg/kg, 2.5 mg/kg, 3 mg/kg, 3.5 mg/kg, 4.0 mg/kg, 4.5 mg/kg, 5 mg/kg, 5.5 mg/kg, 6 mg/kg, 6.5 mg/kg, 7 mg/kg, 7.5 mg/kg, 8 mg/kg, 8.5 mg/kg, 9 mg/kg, 9.5 mg/kg, 10 mg/kg 10.5 mg/kg, 11 mg/kg, 11.5 mg/kg, 12 mg/kg, 12.5 mg/kg, 13 mg/kg, 13.5 mg/kg, 14 mg/kg, 14.5 mg/kg, 15 mg/kg, 16 mg/kg, 17 mg/kg, 18 mg/kg, 19 mg/kg, 20 mg/kg, 21 mg/kg, 22 mg/kg, 23 mg/kg, 24 mg/kg, 25 mg/kg, 30 mg/kg, 35 mg/kg, 40 mg/kg, 45 mg/kg, 50 mg/kg, 55 mg/kg, 60 mg/kg, 65 mg/kg, 70 mg/kg, 75 mg/kg, 80 mg/kg, 85 mg/kg, 90 mg/kg, 95 mg/kg, 100 mg/kg, 105 mg/kg, 110 mg/kg, 115 mg/kg, 120 mg/kg, 125 mg/kg, 130 mg/kg, 135 mg/kg, 140 mg/kg, 145 mg/kg, 150 mg/kg, 155 mg/kg, 160 mg/kg, 165 mg/kg, 170 mg/kg, 175 mg/kg, 180 mg/kg, 185 mg/kg, 190 mg/kg, 195 mg/kg, 200 mg/kg, and any fraction or integer in between any two of the preceding amounts.

In some embodiments, a NOS inhibitor composition comprising BYK-191023 and/or a salt thereof, such as, for example, BYK-191023 dihydrochloride, may be administered as a suitable dosage form, as described herein, to treat, ameliorate, and/or reduce the severity of intestinal adenoma or one or more symptoms thereof in an intestinal adenoma subject. In certain embodiments, an nNOS inhibitor composition comprising BYK-191023 and/or a salt thereof, such as, for example, BYK-191023 dihydrochloride, may be administered to an intestinal adenoma subject to treat, ameliorate, and/or reduce the severity of intestinal adenoma or one or more symptoms thereof in the intestinal adenoma subject: (i) via a route selected from the group consisting of buccally, inhalation, intraarterially, intraarticularly, intracardially, intracerebroventricularly, intradermally, intramedullary, intramuscularly, intraperitoneally, intrathecally, intravenously, intraventricularly, nasally, ophthalmically, orally, rectally, sublabially, sublingually, subcutaneously, topically, and transdermally; and/or (ii) with a frequency selected from the group consisting of four times daily, three times daily, twice daily, once daily, once every two days, once every three days, once every four days, once every five days, once every six days, once a week, once every eight days, once every nine days, once every ten days, once every two weeks, once every three weeks, and once a month; and/or (iii) at a dosage (based on subject weight) selected from the group consisting of 0.1 μg/kg, 0.5 μg/kg, 1 μg/kg, 1.5 μg/kg, 2.0 μg/kg, 2.5 μg/kg, 3.0 μg/kg, 3.5 μg/kg, 4.0 μg/kg, 4.5 μg/kg, 5.0 μg/kg, 10 μg/kg, 15 μg/kg, 20 μg/kg, 25 μg/kg, 30 μg/kg, 35 μg/kg, 40 μg/kg, 45 μg/kg, 50 μg/kg, 55 μg/kg, 60 μg/kg, 65 μg/kg, 70 μg/kg, 75 μg/kg, 80 μg/kg, 85 μg/kg, 90 μg/kg, 95 μg/kg, 100 μg/kg, 150 μg/kg, 200 μg/kg, 250 μg/kg, 300 μg/kg, 350 μg/kg, 400 μg/kg, 450 μg/kg, 500 μg/kg, 550 μg/kg, 600 μg/kg, 650 μg/kg, 700 μg/kg, 750 μg/kg, 800 μg/kg, 850 μg/kg, 900 μg/kg, 1 mg/kg, 1.5 mg/kg, 2.0 mg/kg, 2.5 mg/kg, 3 mg/kg, 3.5 mg/kg, 4.0 mg/kg, 4.5 mg/kg, 5 mg/kg, 5.5 mg/kg, 6 mg/kg, 6.5 mg/kg, 7 mg/kg, 7.5 mg/kg, 8 mg/kg, 8.5 mg/kg, 9 mg/kg, 9.5 mg/kg, 10 mg/kg 10.5 mg/kg, 11 mg/kg, 11.5 mg/kg, 12 mg/kg, 12.5 mg/kg, 13 mg/kg, 13.5 mg/kg, 14 mg/kg, 14.5 mg/kg, 15 mg/kg, 16 mg/kg, 17 mg/kg, 18 mg/kg, 19 mg/kg, 20 mg/kg, 21 mg/kg, 22 mg/kg, 23 mg/kg, 24 mg/kg, 25 mg/kg, 30 mg/kg, 35 mg/kg, 40 mg/kg, 45 mg/kg, 50 mg/kg, 55 mg/kg, 60 mg/kg, 65 mg/kg, 70 mg/kg, 75 mg/kg, 80 mg/kg, 85 mg/kg, 90 mg/kg, 95 mg/kg, 100 mg/kg, 105 mg/kg, 110 mg/kg, 115 mg/kg, 120 mg/kg, 125 mg/kg, 130 mg/kg, 135 mg/kg, 140 mg/kg, 145 mg/kg, 150 mg/kg, 155 mg/kg, 160 mg/kg, 165 mg/kg, 170 mg/kg, 175 mg/kg, 180 mg/kg, 185 mg/kg, 190 mg/kg, 195 mg/kg, 200 mg/kg, and any fraction or integer in between any two of the preceding amounts.

In certain embodiments, a NOS inhibitor composition comprising CM544 and/or a salt thereof, such as, for example, CM544 dihydrochloride, may be administered as a suitable dosage form, as described herein, to treat, ameliorate, and/or reduce the severity of intestinal adenoma or one or more symptoms thereof in an intestinal adenoma subject. In some embodiments, an nNOS inhibitor composition comprising CM544 and/or a salt thereof, such as, for example, CM544 dihydrochloride, may be administered to an intestinal adenoma subject to treat, ameliorate, and/or reduce the severity of intestinal adenoma or one or more symptoms thereof in the intestinal adenoma subject: (i) via a route selected from the group consisting of buccally, inhalation, intraarterially, intraarticularly, intracardially, intracerebroventricularly, intradermally, intramedullary, intramuscularly, intraperitoneally, intrathecally, intravenously, intraventricularly, nasally, ophthalmically, orally, rectally, sublabially, sublingually, subcutaneously, topically, and transdermally; and/or (ii) with a frequency selected from the group consisting of four times daily, three times daily, twice daily, once daily, once every two days, once every three days, once every four days, once every five days, once every six days, once a week, once every eight days, once every nine days, once every ten days, once every two weeks, once every three weeks, and once a month; and/or (iii) at a dosage (based on subject weight) selected from the group consisting of 0.1 μg/kg, 0.5 μg/kg, 1 μg/kg, 1.5 μg/kg, 2.0 μg/kg, 2.5 μg/kg, 3.0 μg/kg, 3.5 μg/kg, 4.0 μg/kg, 4.5 μg/kg, 5.0 μg/kg, 10 μg/kg, 15 μg/kg, 20 μg/kg, 25 μg/kg, 30 μg/kg, 35 μg/kg, 40 μg/kg, 45 μg/kg, 50 μg/kg, 55 μg/kg, 60 μg/kg, 65 μg/kg, 70 μg/kg, 75 μg/kg, 80 μg/kg, 85 μg/kg, 90 μg/kg, 95 μg/kg, 100 μg/kg, 150 μg/kg, 200 μg/kg, 250 μg/kg, 300 μg/kg, 350 μg/kg, 400 μg/kg, 450 μg/kg, 500 μg/kg, 550 μg/kg, 600 μg/kg, 650 μg/kg, 700 μg/kg, 750 μg/kg, 800 μg/kg, 850 μg/kg, 900 μg/kg, 1 mg/kg, 1.5 mg/kg, 2.0 mg/kg, 2.5 mg/kg, 3 mg/kg, 3.5 mg/kg, 4.0 mg/kg, 4.5 mg/kg, 5 mg/kg, 5.5 mg/kg, 6 mg/kg, 6.5 mg/kg, 7 mg/kg, 7.5 mg/kg, 8 mg/kg, 8.5 mg/kg, 9 mg/kg, 9.5 mg/kg, 10 mg/kg 10.5 mg/kg, 11 mg/kg, 11.5 mg/kg, 12 mg/kg, 12.5 mg/kg, 13 mg/kg, 13.5 mg/kg, 14 mg/kg, 14.5 mg/kg, 15 mg/kg, 16 mg/kg, 17 mg/kg, 18 mg/kg, 19 mg/kg, 20 mg/kg, 21 mg/kg, 22 mg/kg, 23 mg/kg, 24 mg/kg, 25 mg/kg, 30 mg/kg, 35 mg/kg, 40 mg/kg, 45 mg/kg, 50 mg/kg, 55 mg/kg, 60 mg/kg, 65 mg/kg, 70 mg/kg, 75 mg/kg, 80 mg/kg, 85 mg/kg, 90 mg/kg, 95 mg/kg, 100 mg/kg, 105 mg/kg, 110 mg/kg, 115 mg/kg, 120 mg/kg, 125 mg/kg, 130 mg/kg, 135 mg/kg, 140 mg/kg, 145 mg/kg, 150 mg/kg, 155 mg/kg, 160 mg/kg, 165 mg/kg, 170 mg/kg, 175 mg/kg, 180 mg/kg, 185 mg/kg, 190 mg/kg, 195 mg/kg, 200 mg/kg, and any fraction or integer in between any two of the preceding amounts.

In certain embodiments, an nNOS inhibitor composition comprising one or more dsRNAs each comprising at least 30 base pairs, may be administered as a suitable dosage form, as described herein, to treat, ameliorate, and/or reduce the severity of intestinal adenoma or one or more symptoms thereof in an intestinal adenoma subject. In some embodiments, an nNOS inhibitor composition comprising one or more dsRNAs, each comprising at least 30 base pairs, may be administered to an intestinal adenoma subject to treat, ameliorate, and/or reduce the severity of intestinal adenoma or one or more symptoms thereof in the intestinal adenoma subject: (i) via a route selected from the group consisting of buccally, inhalation, intraarterially, intraarticularly, intracardially, intracerebroventricularly, intradermally, intramedullary, intramuscularly, intraperitoneally, intrathecally, intravenously, intraventricularly, nasally, ophthalmically, orally, rectally, sublabially, sublingually, subcutaneously, topically, and transdermally; and/or (ii) with a frequency selected from the group consisting of four times daily, three times daily, twice daily, once daily, once every two days, once every three days, once every four days, once every five days, once every six days, once a week, once every eight days, once every nine days, once every ten days, once every two weeks, once every three weeks, and once a month; and/or (iii) at a dosage (based on subject weight) selected from the group consisting of 0.1 μg/kg, 0.5 μg/kg, 1 μg/kg, 1.5 μg/kg, 2.0 μg/kg, 2.5 μg/kg, 3.0 μg/kg, 3.5 μg/kg, 4.0 μg/kg, 4.5 μg/kg, 5.0 μg/kg, 10 μg/kg, 15 μg/kg, 20 μg/kg, 25 μg/kg, 30 μg/kg, 35 μg/kg, 40 μg/kg, 45 μg/kg, 50 μg/kg, 55 μg/kg, 60 μg/kg, 65 μg/kg, 70 μg/kg, 75 μg/kg, 80 μg/kg, 85 μg/kg, 90 μg/kg, 95 μg/kg, 100 μg/kg, 150 μg/kg, 200 μg/kg, 250 μg/kg, 300 μg/kg, 350 μg/kg, 400 μg/kg, 450 μg/kg, 500 μg/kg, 550 μg/kg, 600 μg/kg, 650 μg/kg, 700 μg/kg, 750 μg/kg, 800 μg/kg, 850 μg/kg, 900 μg/kg, 1 mg/kg, 1.5 mg/kg, 2.0 mg/kg, 2.5 mg/kg, 3 mg/kg, 3.5 mg/kg, 4.0 mg/kg, 4.5 mg/kg, 5 mg/kg, 5.5 mg/kg, 6 mg/kg, 6.5 mg/kg, 7 mg/kg, 7.5 mg/kg, 8 mg/kg, 8.5 mg/kg, 9 mg/kg, 9.5 mg/kg, 10 mg/kg 10.5 mg/kg, 11 mg/kg, 11.5 mg/kg, 12 mg/kg, 12.5 mg/kg, 13 mg/kg, 13.5 mg/kg, 14 mg/kg, 14.5 mg/kg, 15 mg/kg, 16 mg/kg, 17 mg/kg, 18 mg/kg, 19 mg/kg, 20 mg/kg, 21 mg/kg, 22 mg/kg, 23 mg/kg, 24 mg/kg, 25 mg/kg, 30 mg/kg, 35 mg/kg, 40 mg/kg, 45 mg/kg, 50 mg/kg, 55 mg/kg, 60 mg/kg, 65 mg/kg, 70 mg/kg, 75 mg/kg, 80 mg/kg, 85 mg/kg, 90 mg/kg, 95 mg/kg, 100 mg/kg, 105 mg/kg, 110 mg/kg, 115 mg/kg, 120 mg/kg, 125 mg/kg, 130 mg/kg, 135 mg/kg, 140 mg/kg, 145 mg/kg, 150 mg/kg, 155 mg/kg, 160 mg/kg, 165 mg/kg, 170 mg/kg, 175 mg/kg, 180 mg/kg, 185 mg/kg, 190 mg/kg, 195 mg/kg, 200 mg/kg, and any fraction or integer in between any two of the preceding amounts.

In some embodiments, an nNOS inhibitor composition comprising one or more siRNAs, may be administered as a suitable dosage form, as described herein, to treat, ameliorate, and/or reduce the severity of intestinal adenoma or one or more symptoms thereof in an intestinal adenoma subject. In certain embodiments, an nNOS inhibitor composition comprising one or more siRNAs selected from the group consisting of SEQ ID NO. 4, SEQ ID NO. 5, SEQ ID NO. 6, SEQ ID NO. 7, SEQ ID NO. 8, and any combination thereof, may be administered as a suitable dosage form, as described herein, to treat, ameliorate, and/or reduce the severity of intestinal adenoma or one or more symptoms thereof in an intestinal adenoma subject. In some embodiments, an nNOS inhibitor composition comprising one or more siRNAs selected from the group consisting of SEQ ID NO. 4, SEQ ID NO. 5, SEQ ID NO. 6, SEQ ID NO. 7, SEQ ID NO. 8, and any combination thereof, may be administered to an intestinal adenoma subject to treat, ameliorate, and/or reduce the severity of intestinal adenoma or one or more symptoms thereof in the intestinal adenoma subject: (i) via a route selected from the group consisting of buccally, inhalation, intraarterially, intraarticularly, intracardially, intracerebroventricularly, intradermally, intramedullary, intramuscularly, intraperitoneally, intrathecally, intravenously, intraventricularly, nasally, ophthalmically, orally, rectally, sublabially, sublingually, subcutaneously, topically, and transdermally; and/or (ii) with a frequency selected from the group consisting of four times daily, three times daily, twice daily, once daily, once every two days, once every three days, once every four days, once every five days, once every six days, once a week, once every eight days, once every nine days, once every ten days, once every two weeks, once every three weeks, and once a month; and/or (iii) at a dosage (based on subject weight) selected from the group consisting of 0.1 μg/kg, 0.5 μg/kg, 1 μg/kg, 1.5 μg/kg, 2.0 μg/kg, 2.5 μg/kg, 3.0 μg/kg, 3.5 μg/kg, 4.0 μg/kg, 4.5 μg/kg, 5.0 μg/kg, 10 μg/kg, 15 μg/kg, 20 μg/kg, 25 μg/kg, 30 μg/kg, 35 μg/kg, 40 μg/kg, 45 μg/kg, 50 μg/kg, 55 μg/kg, 60 μg/kg, 65 μg/kg, 70 μg/kg, 75 μg/kg, 80 μg/kg, 85 μg/kg, 90 μg/kg, 95 μg/kg, 100 μg/kg, 150 μg/kg, 200 μg/kg, 250 μg/kg, 300 μg/kg, 350 μg/kg, 400 μg/kg, 450 μg/kg, 500 μg/kg, 550 μg/kg, 600 μg/kg, 650 μg/kg, 700 μg/kg, 750 μg/kg, 800 μg/kg, 850 μg/kg, 900 μg/kg, 1 mg/kg, 1.5 mg/kg, 2.0 mg/kg, 2.5 mg/kg, 3 mg/kg, 3.5 mg/kg, 4.0 mg/kg, 4.5 mg/kg, 5 mg/kg, 5.5 mg/kg, 6 mg/kg, 6.5 mg/kg, 7 mg/kg, 7.5 mg/kg, 8 mg/kg, 8.5 mg/kg, 9 mg/kg, 9.5 mg/kg, 10 mg/kg 10.5 mg/kg, 11 mg/kg, 11.5 mg/kg, 12 mg/kg, 12.5 mg/kg, 13 mg/kg, 13.5 mg/kg, 14 mg/kg, 14.5 mg/kg, 15 mg/kg, 16 mg/kg, 17 mg/kg, 18 mg/kg, 19 mg/kg, 20 mg/kg, 21 mg/kg, 22 mg/kg, 23 mg/kg, 24 mg/kg, 25 mg/kg, 30 mg/kg, 35 mg/kg, 40 mg/kg, 45 mg/kg, 50 mg/kg, 55 mg/kg, 60 mg/kg, 65 mg/kg, 70 mg/kg, 75 mg/kg, 80 mg/kg, 85 mg/kg, 90 mg/kg, 95 mg/kg, 100 mg/kg, 105 mg/kg, 110 mg/kg, 115 mg/kg, 120 mg/kg, 125 mg/kg, 130 mg/kg, 135 mg/kg, 140 mg/kg, 145 mg/kg, 150 mg/kg, 155 mg/kg, 160 mg/kg, 165 mg/kg, 170 mg/kg, 175 mg/kg, 180 mg/kg, 185 mg/kg, 190 mg/kg, 195 mg/kg, 200 mg/kg, and any fraction or integer in between any two of the preceding amounts.

In some embodiments, an nNOS inhibitor composition comprising one or more shRNAs, may be administered as a suitable dosage form, as described herein, to treat, ameliorate, and/or reduce the severity of intestinal adenoma or one or more symptoms thereof in an intestinal adenoma subject. In certain embodiments, an nNOS inhibitor composition comprising one or more shRNAs selected from the group consisting of SEQ ID NO. 9, SEQ ID NO. 10, SEQ ID NO. 11, SEQ ID NO. 12, SEQ ID NO. 13, SEQ ID NO. 14, and any combination thereof, may be administered as a suitable dosage form, as described herein, to treat, ameliorate, and/or reduce the severity of intestinal adenoma or one or more symptoms thereof in an intestinal adenoma subject. In some embodiments, an nNOS inhibitor composition comprising one or more shRNAs selected from the group consisting of SEQ ID NO. 9, SEQ ID NO. 10, SEQ ID NO. 11, SEQ ID NO. 12, SEQ ID NO. 13, SEQ ID NO. 14, and any combination thereof, may be administered to an intestinal adenoma subject to treat, ameliorate, and/or reduce the severity of intestinal adenoma or one or more symptoms thereof in the intestinal adenoma subject: (i) via a route selected from the group consisting of buccally, inhalation, intraarterially, intraarticularly, intracardially, intracerebroventricularly, intradermally, intramedullary, intramuscularly, intraperitoneally, intrathecally, intravenously, intraventricularly, nasally, ophthalmically, orally, rectally, sublabially, sublingually, subcutaneously, topically, and transdermally; and/or (ii) with a frequency selected from the group consisting of four times daily, three times daily, twice daily, once daily, once every two days, once every three days, once every four days, once every five days, once every six days, once a week, once every eight days, once every nine days, once every ten days, once every two weeks, once every three weeks, and once a month; and/or (iii) at a dosage (based on subject weight) selected from the group consisting of 0.1 μg/kg, 0.5 μg/kg, 1 μg/kg, 1.5 μg/kg, 2.0 μg/kg, 2.5 μg/kg, 3.0 μg/kg, 3.5 μg/kg, 4.0 μg/kg, 4.5 μg/kg, 5.0 μg/kg, 10 μg/kg, 15 μg/kg, 20 μg/kg, 25 μg/kg, 30 μg/kg, 35 μg/kg, 40 μg/kg, 45 μg/kg, 50 μg/kg, 55 μg/kg, 60 μg/kg, 65 μg/kg, 70 μg/kg, 75 μg/kg, 80 μg/kg, 85 μg/kg, 90 μg/kg, 95 μg/kg, 100 μg/kg, 150 μg/kg, 200 μg/kg, 250 μg/kg, 300 μg/kg, 350 μg/kg, 400 μg/kg, 450 μg/kg, 500 μg/kg, 550 μg/kg, 600 μg/kg, 650 μg/kg, 700 μg/kg, 750 μg/kg, 800 μg/kg, 850 μg/kg, 900 μg/kg, 1 mg/kg, 1.5 mg/kg, 2.0 mg/kg, 2.5 mg/kg, 3 mg/kg, 3.5 mg/kg, 4.0 mg/kg, 4.5 mg/kg, 5 mg/kg, 5.5 mg/kg, 6 mg/kg, 6.5 mg/kg, 7 mg/kg, 7.5 mg/kg, 8 mg/kg, 8.5 mg/kg, 9 mg/kg, 9.5 mg/kg, 10 mg/kg 10.5 mg/kg, 11 mg/kg, 11.5 mg/kg, 12 mg/kg, 12.5 mg/kg, 13 mg/kg, 13.5 mg/kg, 14 mg/kg, 14.5 mg/kg, 15 mg/kg, 16 mg/kg, 17 mg/kg, 18 mg/kg, 19 mg/kg, 20 mg/kg, 21 mg/kg, 22 mg/kg, 23 mg/kg, 24 mg/kg, 25 mg/kg, 30 mg/kg, 35 mg/kg, 40 mg/kg, 45 mg/kg, 50 mg/kg, 55 mg/kg, 60 mg/kg, 65 mg/kg, 70 mg/kg, 75 mg/kg, 80 mg/kg, 85 mg/kg, 90 mg/kg, 95 mg/kg, 100 mg/kg, 105 mg/kg, 110 mg/kg, 115 mg/kg, 120 mg/kg, 125 mg/kg, 130 mg/kg, 135 mg/kg, 140 mg/kg, 145 mg/kg, 150 mg/kg, 155 mg/kg, 160 mg/kg, 165 mg/kg, 170 mg/kg, 175 mg/kg, 180 mg/kg, 185 mg/kg, 190 mg/kg, 195 mg/kg, 200 mg/kg, and any fraction or integer in between any two of the preceding amounts.

In some embodiments, an nNOS inhibitor composition comprising one or more miRNAs, may be administered as a suitable dosage form, as described herein, to treat, ameliorate, and/or reduce the severity of intestinal adenoma or one or more symptoms thereof in an intestinal adenoma subject. In certain embodiments, an nNOS inhibitor composition comprising one or more miRNAs selected from the group consisting of miR-146a, hsa-miR-146a-5p, hsa-miR-1273f, hsa-miR-143-3p, hsa-miR-147a, hsa-miR-3622a-5p, hsa-miR-3911, hsa-miR-4269, hsa-miR-4708-5p, hsa-miR-4710, hsa-miR-4770, hsa-miR-4792, hsa-miR-574-5p, hsa-miR-6088, hsa-miR-644a, hsa-miR-6715b-5p, hsa-miR-6768-5p, hsa-miR-6867-5p, hsa-miR-7855-5p, hsa-miR-8485, and any combination thereof, may be administered as a suitable dosage form, as described herein, to treat, ameliorate, and/or reduce the severity of intestinal adenoma or one or more symptoms thereof in an intestinal adenoma subject. In some embodiments, an nNOS inhibitor composition comprising one or more miRNAs selected from the group consisting of miR-146a, hsa-miR-146a-5p, hsa-miR-1273f, hsa-miR-143-3p, hsa-miR-147a, hsa-miR-3622a-5p, hsa-miR-3911, hsa-miR-4269, hsa-miR-4708-5p, hsa-miR-4710, hsa-miR-4770, hsa-miR-4792, hsa-miR-574-5p, hsa-miR-6088, hsa-miR-644a, hsa-miR-6715b-5p, hsa-miR-6768-5p, hsa-miR-6867-5p, hsa-miR-7855-5p, hsa-miR-8485, and any combination thereof, may be administered to an intestinal adenoma subject to treat, ameliorate, and/or reduce the severity of intestinal adenoma or one or more symptoms thereof in the intestinal adenoma subject: (i) via a route selected from the group consisting of buccally, inhalation, intraarterially, intraarticularly, intracardially, intracerebroventricularly, intradermally, intramedullary, intramuscularly, intraperitoneally, intrathecally, intravenously, intraventricularly, nasally, ophthalmically, orally, rectally, sublabially, sublingually, subcutaneously, topically, and transdermally; and/or (ii) with a frequency selected from the group consisting of four times daily, three times daily, twice daily, once daily, once every two days, once every three days, once every four days, once every five days, once every six days, once a week, once every eight days, once every nine days, once every ten days, once every two weeks, once every three weeks, and once a month; and/or (iii) at a dosage (based on subject weight) selected from the group consisting of 0.1 μg/kg, 0.5 μg/kg, 1 μg/kg, 1.5 μg/kg, 2.0 μg/kg, 2.5 μg/kg, 3.0 μg/kg, 3.5 μg/kg, 4.0 μg/kg, 4.5 μg/kg, 5.0 μg/kg, 10 μg/kg, 15 μg/kg, 20 μg/kg, 25 μg/kg, 30 μg/kg, 35 μg/kg, 40 μg/kg, 45 μg/kg, 50 μg/kg, 55 μg/kg, 60 μg/kg, 65 μg/kg, 70 μg/kg, 75 μg/kg, 80 μg/kg, 85 μg/kg, 90 μg/kg, 95 μg/kg, 100 μg/kg, 150 μg/kg, 200 μg/kg, 250 μg/kg, 300 μg/kg, 350 μg/kg, 400 μg/kg, 450 μg/kg, 500 μg/kg, 550 μg/kg, 600 μg/kg, 650 μg/kg, 700 μg/kg, 750 μg/kg, 800 μg/kg, 850 μg/kg, 900 μg/kg, 1 mg/kg, 1.5 mg/kg, 2.0 mg/kg, 2.5 mg/kg, 3 mg/kg, 3.5 mg/kg, 4.0 mg/kg, 4.5 mg/kg, 5 mg/kg, 5.5 mg/kg, 6 mg/kg, 6.5 mg/kg, 7 mg/kg, 7.5 mg/kg, 8 mg/kg, 8.5 mg/kg, 9 mg/kg, 9.5 mg/kg, 10 mg/kg 10.5 mg/kg, 11 mg/kg, 11.5 mg/kg, 12 mg/kg, 12.5 mg/kg, 13 mg/kg, 13.5 mg/kg, 14 mg/kg, 14.5 mg/kg, 15 mg/kg, 16 mg/kg, 17 mg/kg, 18 mg/kg, 19 mg/kg, 20 mg/kg, 21 mg/kg, 22 mg/kg, 23 mg/kg, 24 mg/kg, 25 mg/kg, 30 mg/kg, 35 mg/kg, 40 mg/kg, 45 mg/kg, 50 mg/kg, 55 mg/kg, 60 mg/kg, 65 mg/kg, 70 mg/kg, 75 mg/kg, 80 mg/kg, 85 mg/kg, 90 mg/kg, 95 mg/kg, 100 mg/kg, 105 mg/kg, 110 mg/kg, 115 mg/kg, 120 mg/kg, 125 mg/kg, 130 mg/kg, 135 mg/kg, 140 mg/kg, 145 mg/kg, 150 mg/kg, 155 mg/kg, 160 mg/kg, 165 mg/kg, 170 mg/kg, 175 mg/kg, 180 mg/kg, 185 mg/kg, 190 mg/kg, 195 mg/kg, 200 mg/kg, and any fraction or integer in between any two of the preceding amounts.

In some embodiments, an nNOS inhibitor composition comprising one or more CRISPR/Cas systems, may be administered as a suitable dosage form, as described herein, to treat, ameliorate, and/or reduce the severity of intestinal adenoma or one or more symptoms thereof in an intestinal adenoma subject. In certain embodiments, an nNOS inhibitor composition comprising one or more CRISPR/Cas systems comprising an endonuclease selected from the group consisting of Cas1, Cas2, Cas3, Cas4, Cas5, Cas6, Cas7, Cas8a, Cas8b, Cas8c, Cas9, Cas10, Cas10d, Cas12, Cas12a, Cas12b, Cas12c, Cas12d, Cas12e, Cas12f, Cas12g, Cas12h, Cas12i, Cas12k, C2c4, C2c8, C2c9, Cas13, Cas13a, Cas13b, Cas13c, Cas13d, Cas13x.1, Cse1, Cse2, Csy1, Csy2, Csy3, GSU0054, Csm2, Cmr5, Csx10, Csx11, Csf1, and Csn2, may be administered as a suitable dosage form, as described herein, to treat, ameliorate, and/or reduce the severity of intestinal adenoma or one or more symptoms thereof in an intestinal adenoma subject. In some embodiments, an nNOS inhibitor composition comprising one or more CRISPR/Cas systems comprising an endonuclease selected from the group consisting of Cas1, Cas2, Cas3, Cas4, Cas5, Cas6, Cas7, Cas8a, Cas8b, Cas8c, Cas9, Cas10, Cas10d, Cas12, Cas12a, Cas12b, Cas12c, Cas12d, Cas12e, Cas12f, Cas12g, Cas12h, Cas12i, Cas12k, C2c4, C2c8, C2c9, Cas13, Cas13a, Cas13b, Cas13c, Cas13d, Cas13x.1, Cse1, Cse2, Csy1, Csy2, Csy3, GSU0054, Csm2, Cmr5, Csx10, Csx11, Csf1, and Csn2, may be administered to an intestinal adenoma subject to treat, ameliorate, and/or reduce the severity of intestinal adenoma or one or more symptoms thereof in the intestinal adenoma subject: (i) via a route selected from the group consisting of buccally, inhalation, intraarterially, intraarticularly, intracardially, intracerebroventricularly, intradermally, intramedullary, intramuscularly, intraperitoneally, intrathecally, intravenously, intraventricularly, nasally, ophthalmically, orally, rectally, sublabially, sublingually, subcutaneously, topically, and transdermally; and/or (ii) with a frequency selected from the group consisting of four times daily, three times daily, twice daily, once daily, once every two days, once every three days, once every four days, once every five days, once every six days, once a week, once every eight days, once every nine days, once every ten days, once every two weeks, once every three weeks, and once a month; and/or (iii) at a dosage (based on subject weight) selected from the group consisting of 0.1 μg/kg, 0.5 μg/kg, 1 μg/kg, 1.5 μg/kg, 2.0 μg/kg, 2.5 μg/kg, 3.0 μg/kg, 3.5 μg/kg, 4.0 μg/kg, 4.5 μg/kg, 5.0 μg/kg, 10 μg/kg, 15 μg/kg, 20 μg/kg, 25 μg/kg, 30 μg/kg, 35 μg/kg, 40 μg/kg, 45 μg/kg, 50 μg/kg, 55 μg/kg, 60 μg/kg, 65 μg/kg, 70 μg/kg, 75 μg/kg, 80 μg/kg, 85 μg/kg, 90 μg/kg, 95 μg/kg, 100 μg/kg, 150 μg/kg, 200 μg/kg, 250 μg/kg, 300 μg/kg, 350 μg/kg, 400 μg/kg, 450 μg/kg, 500 μg/kg, 550 μg/kg, 600 μg/kg, 650 μg/kg, 700 μg/kg, 750 μg/kg, 800 μg/kg, 850 μg/kg, 900 μg/kg, 1 mg/kg, 1.5 mg/kg, 2.0 mg/kg, 2.5 mg/kg, 3 mg/kg, 3.5 mg/kg, 4.0 mg/kg, 4.5 mg/kg, 5 mg/kg, 5.5 mg/kg, 6 mg/kg, 6.5 mg/kg, 7 mg/kg, 7.5 mg/kg, 8 mg/kg, 8.5 mg/kg, 9 mg/kg, 9.5 mg/kg, 10 mg/kg 10.5 mg/kg, 11 mg/kg, 11.5 mg/kg, 12 mg/kg, 12.5 mg/kg, 13 mg/kg, 13.5 mg/kg, 14 mg/kg, 14.5 mg/kg, 15 mg/kg, 16 mg/kg, 17 mg/kg, 18 mg/kg, 19 mg/kg, 20 mg/kg, 21 mg/kg, 22 mg/kg, 23 mg/kg, 24 mg/kg, 25 mg/kg, 30 mg/kg, 35 mg/kg, 40 mg/kg, 45 mg/kg, 50 mg/kg, 55 mg/kg, 60 mg/kg, 65 mg/kg, 70 mg/kg, 75 mg/kg, 80 mg/kg, 85 mg/kg, 90 mg/kg, 95 mg/kg, 100 mg/kg, 105 mg/kg, 110 mg/kg, 115 mg/kg, 120 mg/kg, 125 mg/kg, 130 mg/kg, 135 mg/kg, 140 mg/kg, 145 mg/kg, 150 mg/kg, 155 mg/kg, 160 mg/kg, 165 mg/kg, 170 mg/kg, 175 mg/kg, 180 mg/kg, 185 mg/kg, 190 mg/kg, 195 mg/kg, 200 mg/kg, and any fraction or integer in between any two of the preceding amounts.

In some embodiments, an nNOS inhibitor composition comprising one or more antibodies, may be administered as a suitable dosage form, as described herein, to treat, ameliorate, and/or reduce the severity of intestinal adenoma or one or more symptoms thereof in an intestinal adenoma subject. In certain embodiments, an nNOS inhibitor composition comprising one or more antibodies selected from the group consisting of 37-2800 (from ThermoFisher), EP1855Y (from abcam), EPR 24351-6 (from abcam), and any combination thereof, may be administered as a suitable dosage form, as described herein, to treat, ameliorate, and/or reduce the severity of intestinal adenoma or one or more symptoms thereof in an intestinal adenoma subject. In some embodiments, an nNOS inhibitor composition comprising one or more antibodies selected from the group consisting of 37-2800 (from ThermoFisher), EP1855Y (from abeam), EPR 24351-6 (from abeam), and any combination thereof, may be administered to an intestinal adenoma subject to treat, ameliorate, and/or reduce the severity of intestinal adenoma or one or more symptoms thereof in the intestinal adenoma subject: (i) via a route selected from the group consisting of buccally, inhalation, intraarterially, intraarticularly, intracardially, intracerebroventricularly, intradermally, intramedullary, intramuscularly, intraperitoneally, intrathecally, intravenously, intraventricularly, nasally, ophthalmically, orally, rectally, sublabially, sublingually, subcutaneously, topically, and transdermally; and/or (ii) with a frequency selected from the group consisting of four times daily, three times daily, twice daily, once daily, once every two days, once every three days, once every four days, once every five days, once every six days, once a week, once every eight days, once every nine days, once every ten days, once every two weeks, once every three weeks, and once a month; and/or (iii) at a dosage (based on subject weight) selected from the group consisting of 0.1 μg/kg, 0.5 μg/kg, 1 μg/kg, 1.5 μg/kg, 2.0 μg/kg, 2.5 μg/kg, 3.0 μg/kg, 3.5 μg/kg, 4.0 μg/kg, 4.5 μg/kg, 5.0 μg/kg, 10 μg/kg, 15 μg/kg, 20 μg/kg, 25 μg/kg, 30 μg/kg, 35 μg/kg, 40 μg/kg, 45 μg/kg, 50 μg/kg, 55 μg/kg, 60 μg/kg, 65 μg/kg, 70 μg/kg, 75 μg/kg, 80 μg/kg, 85 μg/kg, 90 μg/kg, 95 μg/kg, 100 μg/kg, 150 μg/kg, 200 μg/kg, 250 μg/kg, 300 μg/kg, 350 μg/kg, 400 μg/kg, 450 μg/kg, 500 μg/kg, 550 μg/kg, 600 μg/kg, 650 μg/kg, 700 μg/kg, 750 μg/kg, 800 μg/kg, 850 μg/kg, 900 μg/kg, 1 mg/kg, 1.5 mg/kg, 2.0 mg/kg, 2.5 mg/kg, 3 mg/kg, 3.5 mg/kg, 4.0 mg/kg, 4.5 mg/kg, 5 mg/kg, 5.5 mg/kg, 6 mg/kg, 6.5 mg/kg, 7 mg/kg, 7.5 mg/kg, 8 mg/kg, 8.5 mg/kg, 9 mg/kg, 9.5 mg/kg, 10 mg/kg 10.5 mg/kg, 11 mg/kg, 11.5 mg/kg, 12 mg/kg, 12.5 mg/kg, 13 mg/kg, 13.5 mg/kg, 14 mg/kg, 14.5 mg/kg, 15 mg/kg, 16 mg/kg, 17 mg/kg, 18 mg/kg, 19 mg/kg, 20 mg/kg, 21 mg/kg, 22 mg/kg, 23 mg/kg, 24 mg/kg, 25 mg/kg, 30 mg/kg, 35 mg/kg, 40 mg/kg, 45 mg/kg, 50 mg/kg, 55 mg/kg, 60 mg/kg, 65 mg/kg, 70 mg/kg, 75 mg/kg, 80 mg/kg, 85 mg/kg, 90 mg/kg, 95 mg/kg, 100 mg/kg, 105 mg/kg, 110 mg/kg, 115 mg/kg, 120 mg/kg, 125 mg/kg, 130 mg/kg, 135 mg/kg, 140 mg/kg, 145 mg/kg, 150 mg/kg, 155 mg/kg, 160 mg/kg, 165 mg/kg, 170 mg/kg, 175 mg/kg, 180 mg/kg, 185 mg/kg, 190 mg/kg, 195 mg/kg, 200 mg/kg, and any fraction or integer in between any two of the preceding amounts.

In some embodiments, an nNOS inhibitor composition comprising one or more amino acid-based compounds, may be administered as a suitable dosage form, as described herein, to treat, ameliorate, and/or reduce the severity of intestinal adenoma or one or more symptoms thereof in an intestinal adenoma subject. In certain embodiments, an nNOS inhibitor composition comprising one or more amino acid-compounds that bind an active site of nNOS selected from the group consisting of Phenylalanine 589, Tryptophan 592, Tyrosine 593, Glutamate 597, Aspartate 602, Arginine 601, Methionine 341, Histidine 342, and Tyrosine711, may be administered as a suitable dosage form, as described herein, to treat, ameliorate, and/or reduce the severity of intestinal adenoma or one or more symptoms thereof in an intestinal adenoma subject. In some embodiments, an nNOS inhibitor composition comprising one or more amino acid-compounds that bind an active site of nNOS selected from the group consisting of Phenylalanine 589, Tryptophan 592, Tyrosine 593, Glutamate 597, Aspartate 602, Arginine 601, Methionine 341, Histidine 342, and Tyrosine711, may be administered to an intestinal adenoma subject to treat, ameliorate, and/or reduce the severity of intestinal adenoma or one or more symptoms thereof in the intestinal adenoma subject: (i) via a route selected from the group consisting of buccally, inhalation, intraarterially, intraarticularly, intracardially, intracerebroventricularly, intradermally, intramedullary, intramuscularly, intraperitoneally, intrathecally, intravenously, intraventricularly, nasally, ophthalmically, orally, rectally, sublabially, sublingually, subcutaneously, topically, and transdermally; and/or (ii) with a frequency selected from the group consisting of four times daily, three times daily, twice daily, once daily, once every two days, once every three days, once every four days, once every five days, once every six days, once a week, once every eight days, once every nine days, once every ten days, once every two weeks, once every three weeks, and once a month; and/or (iii) at a dosage (based on subject weight) selected from the group consisting of 0.1 μg/kg, 0.5 μg/kg, 1 μg/kg, 1.5 μg/kg, 2.0 μg/kg, 2.5 μg/kg, 3.0 μg/kg, 3.5 μg/kg, 4.0 μg/kg, 4.5 μg/kg, 5.0 μg/kg, 10 μg/kg, 15 μg/kg, 20 μg/kg, 25 μg/kg, 30 μg/kg, 35 μg/kg, 40 μg/kg, 45 μg/kg, 50 μg/kg, 55 μg/kg, 60 μg/kg, 65 μg/kg, 70 μg/kg, 75 μg/kg, 80 μg/kg, 85 μg/kg, 90 μg/kg, 95 μg/kg, 100 μg/kg, 150 μg/kg, 200 μg/kg, 250 μg/kg, 300 μg/kg, 350 μg/kg, 400 μg/kg, 450 μg/kg, 500 μg/kg, 550 μg/kg, 600 μg/kg, 650 μg/kg, 700 μg/kg, 750 μg/kg, 800 μg/kg, 850 μg/kg, 900 μg/kg, 1 mg/kg, 1.5 mg/kg, 2.0 mg/kg, 2.5 mg/kg, 3 mg/kg, 3.5 mg/kg, 4.0 mg/kg, 4.5 mg/kg, 5 mg/kg, 5.5 mg/kg, 6 mg/kg, 6.5 mg/kg, 7 mg/kg, 7.5 mg/kg, 8 mg/kg, 8.5 mg/kg, 9 mg/kg, 9.5 mg/kg, 10 mg/kg 10.5 mg/kg, 11 mg/kg, 11.5 mg/kg, 12 mg/kg, 12.5 mg/kg, 13 mg/kg, 13.5 mg/kg, 14 mg/kg, 14.5 mg/kg, 15 mg/kg, 16 mg/kg, 17 mg/kg, 18 mg/kg, 19 mg/kg, 20 mg/kg, 21 mg/kg, 22 mg/kg, 23 mg/kg, 24 mg/kg, 25 mg/kg, 30 mg/kg, 35 mg/kg, 40 mg/kg, 45 mg/kg, 50 mg/kg, 55 mg/kg, 60 mg/kg, 65 mg/kg, 70 mg/kg, 75 mg/kg, 80 mg/kg, 85 mg/kg, 90 mg/kg, 95 mg/kg, 100 mg/kg, 105 mg/kg, 110 mg/kg, 115 mg/kg, 120 mg/kg, 125 mg/kg, 130 mg/kg, 135 mg/kg, 140 mg/kg, 145 mg/kg, 150 mg/kg, 155 mg/kg, 160 mg/kg, 165 mg/kg, 170 mg/kg, 175 mg/kg, 180 mg/kg, 185 mg/kg, 190 mg/kg, 195 mg/kg, 200 mg/kg, and any fraction or integer in between any two of the preceding amounts.

In some embodiments, an intestinal adenoma subject, as described herein, may be a female child or a male child. In certain embodiments, an intestinal adenoma subject, as described herein, may be a female adult or a male adult.

In some embodiments, an intestinal adenoma subject, as described herein, may have an abnormal expression of one or more antigens compared to an equivalent subject that does not have an intestinal adenoma. As used herein, the term “equivalent subject,” refers to a subject of the same sex (male/female) and age range (child/adult) of the intestinal adenoma subject. For purposes of disclosure, the term “antigen,” when used with respect to abnormal expression, may define one or more cluster of differentiation (“CD”) antigens, such as, for example, CD15, CD44, CD70, CD133, as well as any CD antigen that has an abnormal expression in an intestinal adenoma that would be readily envisaged by one skilled in the art. In certain embodiments, abnormal expression of an antigen may define an abnormal expression of CD15, an abnormal expression of CD44, an abnormal expression of CD70, an abnormal expression of CD133, an abnormal expression of a CD antigen that has an abnormal expression in an intestinal adenoma that would be readily envisaged by one skilled in the art, and any combination thereof. In some embodiments, an “abnormal expression,” can refer to a reduced expression of an antigen, as described herein. In certain embodiments, an “abnormal expression,” can refer to an overexpression of an antigen, as described herein. Whether “abnormal expression,” refers to a reduced expression of an antigen or an overexpression of an antigen would be immediately envisaged by the skilled artisan when viewing the term in the context of the particular antigen and intestinal adenoma disclosed. For example, in the context of CD24 and colorectal adenoma, one skilled in the art would immediately envisage that “abnormal expression” refers to an overexpression of CD24. Treatment, amelioration, and/or reduction in the severity of an intestinal adenoma or one or more symptoms thereof may be characterized by a restoration to a normal level of expression of one or more antigens from their abnormal state, as described herein. For example, in the context of CD24 and colorectal adenoma, treatment, amelioration, and/or reduction in the severity of an intestinal adenoma or one or more symptoms thereof may be characterized by a reduced expression of CD24.

In certain embodiments, an intestinal adenoma subject, as described herein, may have an abnormal expression of one or more proteins compared to an equivalent subject that does not have an intestinal adenoma. For purposes of disclosure, the term “protein,” when used with respect to abnormal expression, may define one or more proteins and/or enzymes, such as, for example, ALDH1A3, mTOR, p-mTOR, TSC-2, AKT, pAKT, RPS6, p-RPS6, Syp, VEGF, PIGF, as well as any protein or enzyme that has an abnormal expression in an intestinal adenoma that would be readily envisaged by one skilled in the art. In certain embodiments, abnormal expression of a protein may define an abnormal expression of ALDH1A3, an abnormal expression of mTOR, an abnormal expression of p-mTOR, an abnormal expression of TSC-2, an abnormal expression of AKT, an abnormal expression of pAKT, an abnormal expression of RPS6, an abnormal expression of p-RPS6, an abnormal expression of Syp, an abnormal expression of VEGF, an abnormal expression of PIGF, an abnormal expression of a protein or enzyme that has an abnormal expression in an intestinal adenoma that would be readily envisaged by one skilled in the art, and any combination thereof. In some embodiments, an “abnormal expression,” can refer to a reduced expression of a protein, as described herein. In certain embodiments, an “abnormal expression,” can refer to an overexpression of a protein, as described herein. Whether “abnormal expression,” refers to a reduced expression of a protein or an overexpression of a protein would be immediately envisaged by the skilled artisan when viewing the term in the context of the particular protein and intestinal adenoma disclosed. For example, in the context of colorectal adenoma, one skilled in the art would immediately envisage that “abnormal expression” refers to an overexpression of mTOR. Treatment, amelioration, and/or reduction in the severity of an intestinal adenoma or one or more symptoms thereof may be characterized by a restoration to a normal level of expression of one or more proteins from their abnormal state, as described herein. For example, in the context of colorectal adenoma, treatment, amelioration, and/or reduction in the severity of an intestinal adenoma or one or more symptoms thereof may be characterized by a reduced expression of mTOR.

In certain embodiments, an intestinal adenoma subject, as described herein, may have an increased expression of one or more NOS isoforms compared to an equivalent subject that does not have an intestinal adenoma. For purposes of disclosure, the term “NOS isoform,” refers to nNOS, iNOS, and/or eNOS. In certain embodiments, an intestinal adenoma subject, as described herein, may have an increased expression of nNOS compared to an equivalent subject that does not have an intestinal adenoma. In some embodiments, an intestinal adenoma subject, as described herein, may have an increased expression of iNOS compared to an equivalent subject that does not have an intestinal adenoma. In certain embodiments, an intestinal adenoma subject, as described herein, may have an increased expression of eNOS compared to an equivalent subject that does not have an intestinal adenoma. In some embodiments, an intestinal adenoma subject, as described herein, may have an increased expression of nNOS and iNOS compared to an equivalent subject that does not have an intestinal adenoma. In certain embodiments, an intestinal adenoma subject, as described herein, may have an increased expression of nNOS and eNOS compared to an equivalent subject that does not have an intestinal adenoma. In some embodiments, an intestinal adenoma subject, as described herein, may have an increased expression of iNOS and eNOS compared to an equivalent subject that does not have an intestinal adenoma. In certain embodiments, an intestinal adenoma subject, as described herein, may have an increased expression of nNOS, iNOS, and eNOS compared to an equivalent subject that does not have an intestinal adenoma. Whether a “NOS isoform,” has an increased expression would be immediately envisaged by the skilled artisan when viewing the term in the context of the particular NOS isoform and intestinal adenoma. For example, in the context of colorectal adenoma, one skilled in the art would immediately envisage that iNOS has an increased expression. Treatment, amelioration, and/or reduction in the severity of an intestinal adenoma or one or more symptoms thereof may be characterized by a reduced expression of one or more NOS isoforms from their increased state, as described herein. For example, in the context of colorectal adenoma, treatment, amelioration, and/or reduction in the severity of an intestinal adenoma or one or more symptoms thereof may be characterized by a reduced expression of iNOS. For purposes of disclosure, reduction/inhibition of the expression of one or more NOS isoforms may be characterized by a reduction in expression of one or more NOS isoforms by about at least 10%, about at least 20%, about at least 30%, or about at least 40%. In some embodiments, reduction/inhibition of the expression of one or more NOS isoforms may further be characterized by a lack reduction of other, non-implicated NOS isoforms, such as, for example, no more than about 10%, no more than about 5%, no more than about 2%, or no more than about 1% reduction/inhibition of the expression of non-implicated NOS isoforms. As used herein, the term “non-implicated NOS isoforms,” refers to NOS isoforms that do not have an increased expression in an intestinal adenoma. By way of example, in the context of colorectal adenoma, if iNOS has an increased expression, nNOS and/or eNOS would define a “non-implicated” NOS isoform. Whether a NOS isoform is a “non-implicated NOS isoform,” would be immediately envisaged by the skilled artisan when viewing the term in the context of the particular NOS isoform disclosed and an intestinal adenoma.

In some embodiments, an intestinal adenoma subject, as described herein, may have an elevated level of one or more NO-indicators compared to an equivalent subject that does not have an intestinal adenoma. As used herein, the term “NO-indicator,” can refer to 3-nitrotyrosine (3-Ntyr), NADPH-diaphorase, total nitrite concentration, total nitrate concentration, total nitrite and nitrate concentration, as well as any NO-indicator that has an elevated level in an intestinal adenoma that would be readily envisaged by one skilled in the art. For example, in the context of colorectal adenoma, one skilled in the art would immediately envisage that 3-Ntyr can be present at an elevated level. Treatment, amelioration, and/or reduction in the severity of an intestinal adenoma or one or more symptoms thereof may be characterized by a reduction in the level of one or more NO-indicators. For example, in the context of colorectal adenoma, treatment, amelioration, and/or reduction in the severity of an intestinal adenoma or one or more symptoms thereof may be characterized by a reduction in the level of 3-Ntyr.

In certain embodiments, a NOS inhibitor composition, as described herein, may be administered as a suitable dosage form, as described herein, to treat, ameliorate, and/or reduce the severity of an intestinal adenoma or one or more symptoms thereof, as an alternative to a conventional therapy for treating, ameliorating, and/or reducing the severity of an intestinal adenoma or one or more symptoms thereof. For purposes of disclosure, a “conventional therapy,” as described herein, may refer to a treatment that would be recommended by one skilled in the art to treat an intestinal adenoma, and may define one or more of radiation, chemotherapy, immunotherapy, cell-based therapy, gene therapy, and the like—one skilled in the art would immediately envisage a “conventional therapy” based on the particular intestinal adenoma disclosed, as well as characteristics of an intestinal adenoma subject, such as, for example, the subject's sex, age, stage of the intestinal adenoma, genotype of the intestinal adenoma, and the like. Treatment, amelioration, and/or reduction in the severity of an intestinal adenoma or one or more symptoms thereof may be characterized by an improved outcome compared to a conventional therapy, as described herein. For purposes of the disclosure, an “improved outcome” may define any one of the following: (i) a larger reduction in tumor volume and/or mass compared to a subject administered an equivalent dose of a conventional therapy; (ii) an improvement in the expected rate of survival of a subject, at, for example, 1 month, 3 months, 6 months, 9 months, 1 year, 2 years, 3 years, 5 years, 10 years, or more, compared to the expected rate of survival of a subject administered an equivalent dose of a conventional therapy; (iii) a reduction in the prevalence of side effects resulting from the NOS inhibitor composition compared to a subject administered an equivalent dose of a conventional therapy; (iv) a reduction in the severity of side effects resulting from the NOS inhibitor composition compared to a subject administered an equivalent dose of a conventional therapy; (v) a reduction in the dosage required to achieve an equivalent reduction in tumor volume compared to a subject administered a conventional therapy; (vi) a reduction in the dosage required to achieve an equivalent expected rate of survival of a subject compared to a subject administered a conventional therapy; (vii) a reduction in the likelihood of a recurrence of the intestinal adenoma occurring in a subject compared to a subject administered an equivalent dose of a conventional therapy; (viii) a reduction in the likelihood of a metastasis occurring in a subject compared to a subject administered an equivalent dose of a conventional therapy; (ix) a reduction in the time required to be at a medical facility for treatment compared to a subject administered an equivalent dose of a conventional therapy; (x) a reduction in the cost associated with treatment compared to a subject administered an equivalent dose of a conventional therapy; as well as any other improved outcome not expressly recited that would be readily envisaged by one skilled in the art.

In some embodiments, a NOS inhibitor composition, as described herein, may be administered as a suitable dosage form, as described herein, to treat, ameliorate, and/or reduce the severity of an intestinal adenoma or one or more symptoms thereof, wherein the NOS inhibitor composition comprises a first chemical NOS inhibitor and a second chemical NOS inhibitor. In certain embodiments, the first chemical NOS inhibitor may be administered concurrently with the second chemical NOS inhibitor. In some embodiments, the first chemical NOS inhibitor may be administered prior to the second chemical NOS inhibitor. In certain embodiments, the first chemical NOS inhibitor may be administered subsequently to the second chemical NOS inhibitor. Treatment, amelioration, and/or reduction in the severity of an intestinal adenoma or one or more symptoms thereof may be characterized by an improved outcome compared an alternative treatment. As used herein, the term “alternative treatment,” may define one or more of a conventional therapy, as described herein, a NOS inhibitor composition comprising a single chemical NOS inhibitor, as described herein, and a NOS inhibitor composition comprising a single biological NOS inhibitor, as described herein. For purposes of the disclosure, an “improved outcome” may define any one of the following: (i) a larger reduction in tumor volume and/or mass compared an alternative treatment; (ii) an improvement in the expected rate of survival of a subject, at, for example, 1 month, 3 months, 6 months, 9 months, 1 year, 2 years, 3 years, 5 years, 10 years, or more, compared to the expected rate of survival of a subject administered an alternative treatment; (iii) a reduction in the prevalence of side effects resulting from the NOS inhibitor composition comprising at least two chemical NOS inhibitors compared to a subject administered an alternative treatment; (iv) a reduction in the severity of side effects resulting from the NOS inhibitor composition comprising at least two chemical NOS inhibitors compared to a subject administered an alternative treatment; (v) a reduction in the dosage required to achieve an equivalent reduction in tumor volume compared to a subject administered an alternative treatment; (vi) a reduction in the dosage required to achieve an equivalent expected rate of survival of a subject compared to a subject administered an alternative treatment; (vii) a reduction in the likelihood of a recurrence of the intestinal adenoma in a subject compared to a subject administered an alternative treatment; (viii) a reduction in the likelihood of a metastasis occurring in a subject compared to a subject administered an alternative treatment; (ix) a reduction in the time required to be at a medical facility for treatment compared to a subject administered an alternative treatment; (x) a reduction in the cost associated with treatment compared to a subject administered an alternative treatment; as well as any other improved outcome not expressly recited that would be readily envisaged by one skilled in the art.

In certain embodiments, a NOS inhibitor composition, as described herein, may be used to treat, ameliorate, and/or reduce the severity of an intestinal adenoma or one or more symptoms thereof, wherein the NOS inhibitor composition comprises at least one chemical NOS inhibitor and at least one biological NOS inhibitor. In some embodiments, the at least one chemical NOS inhibitor may be administered concurrently with the at least one biological NOS inhibitor. In certain embodiments, the at least one chemical NOS inhibitor may be administered prior to the at least one biological NOS inhibitor. In some embodiments, the at least one chemical NOS inhibitor may be administered subsequently to the at least one biological NOS inhibitor. Treatment, amelioration, and/or reduction in the severity of an intestinal adenoma or one or more symptoms thereof may be characterized by an improved outcome compared an alternative treatment. As used herein, the term “alternative treatment,” may define one or more of a conventional therapy, as described herein, a NOS inhibitor composition comprising a single chemical NOS inhibitor, as described herein, and a NOS inhibitor composition comprising a single biological NOS inhibitor, as described herein. For purposes of the disclosure, an “improved outcome” may define any one of the following: (i) a larger reduction in tumor volume and/or mass compared an alternative treatment; (ii) an improvement in the expected rate of survival of a subject, at, for example, 1 month, 3 months, 6 months, 9 months, 1 year, 2 years, 3 years, 5 years, 10 years, or more, compared to the expected rate of survival of a subject administered an alternative treatment; (iii) a reduction in the prevalence of side effects resulting from the NOS inhibitor composition comprising at least one chemical NOS inhibitor and at least one biological NOS inhibitor compared to a subject administered an alternative treatment; (iv) a reduction in the severity of side effects resulting from the NOS inhibitor composition comprising at least one chemical NOS inhibitor and at least one biological NOS inhibitor compared to a subject administered an alternative treatment; (v) a reduction in the dosage required to achieve an equivalent reduction in tumor volume compared to a subject administered an alternative treatment; (vi) a reduction in the dosage required to achieve an equivalent expected rate of survival of a subject compared to a subject administered an alternative treatment; (vii) a reduction in the likelihood of a recurrence of the intestinal adenoma in a subject compared to a subject administered an alternative treatment; (viii) a reduction in the likelihood of a metastasis occurring in a subject compared to a subject administered an alternative treatment; (ix) a reduction in the time required to be at a medical facility for treatment compared to a subject administered an alternative treatment; (x) a reduction in the cost associated with treatment compared to a subject administered an alternative treatment; as well as any other improved outcome not expressly recited that would be readily envisaged by one skilled in the art.

In some embodiments, a NOS inhibitor composition, as described herein, may be combined with a conventional therapy, as described herein, to treat, ameliorate, and/or reduce the severity of an intestinal adenoma or one or more symptoms thereof. In certain embodiments, the NOS inhibitor composition may be administered concurrently with the conventional therapy. In some embodiments, the NOS inhibitor composition may be administered prior to the conventional therapy. In certain embodiments, the NOS inhibitor composition may be administered subsequently to the conventional therapy. Treatment, amelioration, and/or reduction in the severity of an intestinal adenoma or one or more symptoms thereof may be characterized by an improved outcome compared an alternative treatment. As used herein, the term “alternative treatment,” may define one or more of a conventional therapy, as described herein, a NOS inhibitor composition comprising a single chemical NOS inhibitor, as described herein, and a NOS inhibitor composition comprising a single biological NOS inhibitor, as described herein. For purposes of the disclosure, an “improved outcome” may define any one of the following: (i) a larger reduction in tumor volume and/or mass compared an alternative treatment; (ii) an improvement in the expected rate of survival of a subject, at, for example, 1 month, 3 months, 6 months, 9 months, 1 year, 2 years, 3 years, 5 years, 10 years, or more, compared to the expected rate of survival of a subject administered an alternative treatment; (iii) a reduction in the prevalence of side effects resulting from the NOS inhibitor composition and conventional therapy compared to a subject administered an alternative treatment; (iv) a reduction in the severity of side effects resulting from the NOS inhibitor composition and conventional therapy compared to a subject administered an alternative treatment; (v) a reduction in the dosage required to achieve an equivalent reduction in tumor volume compared to a subject administered an alternative treatment; (vi) a reduction in the dosage required to achieve an equivalent expected rate of survival of a subject compared to a subject administered an alternative treatment; (vii) a reduction in the likelihood of a recurrence of the intestinal adenoma in a subject compared to a subject administered an alternative treatment; (viii) a reduction in the likelihood of a metastasis occurring in a subject compared to a subject administered an alternative treatment; (ix) a reduction in the time required to be at a medical facility for treatment compared to a subject administered an alternative treatment; (x) a reduction in the cost associated with treatment compared to a subject administered an alternative treatment; as well as any other improved outcome not expressly recited that would be readily envisaged by one skilled in the art.

In some embodiments, a “conventional therapy,” as that term is used with respect to combined administration with a NOS inhibitor composition, may refer to a conventional chemotherapy. For purposes of the disclosure, a “conventional chemotherapy,” as described herein may be a compound selected from the group consisting of abemaciclib, abiraterone acetate, acalabrutinib, adagrasib, afatinib dimaleate, albumin-bound paclitaxel, albumin-bound rapamycin (Sirolimus Protein-Bound Particles), alectinib, alpelisib, amifostine, aminolevulinic acid hydrochloride, anastrozole, apalutamide, aprepitant, arsenic trioxide, asciminib hydrochloride, avapritinib, axitinib, azacytidine, belinostat, belzutifan, bendamustine hydrochloride, bexarotene, bicalutamide, binimetinib, bleomycin, bleomycin sulfate, bortezomib, bosutinib, brigatinib, busulfan, cabazitaxel, cabozantinib-S-malate, capecitabine, capmatinib hydrochloride, carboplatin, carfilzomib, carmustine, cedazuridine, ceritinib, chlorambucil, cisplatin, cladribine, clofarabine, cobimetinib fumarate, copanlisib hydrochloride, crizotinib, cyclophosphamide, cytarabine, dabrafenib mesylate, dacarbazine, dacomitinib, dactinomycin, darolutamide, dasatinib, daunorubicin hydrochloride, decitabine, defibrotide sodium, degarelix, dexamethasone, dexrazoxane hydrochloride, docetaxel, doxorubicin hydrochloride, duvelisib, elacestrant dihydrochloride, eltrombopag olamine, enasidenib mesylate, encorafenib, entrectinib, enzalutamide, epirubicin hydrochloride, erdafitinib, eribulin mesylate, erlotinib hydrochloride, etoposide, etoposide phosphate, everolimus, exemestane, fedratinib hydrochloride, fludarabine phosphate, fluorouracil, flutamide, folinic acid, fostamatinib disodium, fulvestrant, futibatinib, gefitinib, gemcitabine hydrochloride, gilteritinib fumarate, glasdegib maleate, goserelin acetate, granisetron, granisetron hydrochloride, hydroxyurea, ibrutinib, idarubicin hydrochloride, idelalisib, ifosfamide, imatinib mesylate, imiquimod, infigratinib phosphate, iobenguane I-131, irinotecan hydrochloride, ivosidenib, ixabepilone, ixazomib citrate, lanreotide acetate, lapatinib ditosylate, larotrectinib sulfate, lenalidomide, lenvatinib mesylate, letrozole, leucovorin calcium, leuprolide acetate, liposomal cytarabine, liposomal doxorubicin hydrochloride, liposomal irinotecan hydrochloride, lomustine, lorlatinib, lurbinectedin, Lu-177 dotatate, Lu-177 vipivotide tetraxetan, mechlorethamine hydrochloride, megestrol acetate, melphalan, melphalan hydrochloride, mercaptopurine, mesna, methotrexate, methotrexate sodium, methylnaltrexone bromide, midostaurin, mitomycin, mitoxantrone hydrochloride, mobocertinib succinate, nelarabine, neratinib maleate, netupitant hydrochloride, nilutamide, nilotinib, niraparib tosylate monohydrate, olaparib, olutasidenib, omacetaxine mepesuccinate, ondansetron hydrochloride, osimertinib mesylate, oxaliplatin, paclitaxel, pacritinib citrate, palbociclib, palonosetron hydrochloride, pamidronate disodium, pazopanib hydrochloride, pemetrexed disodium, pemigatinib, pexidartinib hydrochloride, pirtobrutinib, plerixafor, pomalidomide, ponatinib hydrochloride, pralatrexate, pralsetinib, prednisone, procarbazine hydrochloride, propranolol hydrochloride, Ra-223 dichloride, raloxifene hydrochloride, regorafenib, relugolix, ribociclib, ripretinib, rolapitant hydrochloride, romidepsin, rucaparib camsylate, ruxolitinib phosphate, selinexor, selpercatinib, selumetinib sulfate, sonidegib, sorafenib tosylate, sotorasib, sunitinib malate, talazoparib tosylate, talc, tamoxifen citrate, tazemetostat hydrobromide, temozolomide, temsirolimus, tepotinib hydrochloride, thalidomide, thioguanine, thiotepa, tipiracil hydrochloride, tivozanib hydrochloride, topotecan hydrochloride, toremifene, trabectedin, trametinib dimethyl sulfoxide, tretinoin, trifluridine hydrochloride, tucatinib, uridine triacetate, valrubicin, vandetanib, vemurafenib, venetoclax, vinblastine sulfate, vincristine sulfate, vinorelbine tartrate, vismodegib, vorinostat, zanubrutinib, zoledronic acid, and any combination thereof. One skilled in the art would immediately envisage an appropriate “conventional chemotherapy” based on the particular intestinal adenoma disclosed, as well as characteristics of an intestinal adenoma subject, such as, for example, the subject's sex, age, stage of the intestinal adenoma, genotype of the intestinal adenoma, and the like.

In certain embodiments, a “conventional therapy,” as that term is used with respect to combined administration with a NOS inhibitor composition, may refer to a conventional immunotherapy. For purposes of the disclosure, a “conventional immunotherapy,” as described herein may be an immunotherapy, cell-based therapy, and/or gene-based therapy selected from the group consisting of ado-trastuzumab emtansine, aldesleukin, alemtuzumab, amivantamab-vmjw, asparaginase Erwinia chrysanthemi, asparaginase Erwinia chrysanthemi (recombinant)-rywn, atezolizumab, avelumab, axicabtagene ciloleucel, bevacizumab, blinatumomab, brentuximab vedotin, brexucabtagene autoleucel, calaspargase pegol-mknl, camrelizumab, caplacizumab-yhdp, cemiplimab-rwlc, cetuximab, ciltacabtagene autoleucel, daratumumab, daratumumab and hyaluronidase-fihj, darbepoetin alfa, denileukin diftitox, denosumab, dinutuximab, dostarlimab-gxly, durvalumab, elotuzumab, emapalumab-lzsg, enfortumab vedotin-ejfv, epoetin alfa, fam-trastuzumab deruxtecan-nxki, filgrastim, gemtuzumab ozogamicin, glucarpidase, ibritumomab tiuxetan, idecabtagene vicleucel, inotuzumab ozogamicin, ipilimumab, isatuximab-irfc, lisocabtagene maraleucel, loncastuximab tesirine-lpyl, luspatercept-aamt, margetuximab-cmkb, mirvetuximab soravtansine-gynx, mogamulizumab-kpkc, mosunetuzumab-axgb, moxetumomab pasudotox-tdfk, nadofaragene firadenovec-vncg, naxitamab-gqgk, necitumumab, nivolumab, nivolumab and relatlimab-rmbw, obinutuzumab, ofatumumab, palifermin, panitumumab, pegaspargase, pegfilgrastim, pembrolizumab, pertuzumab, pertuzumab, trastuzumab, and hyaluronidase-zzxf, polatuzumab vedotin-piiq, ramucirumab, rasburicase, ravulizumab-cwvz, recombinant HPV bivalent vaccine, recombinant HPV nonavalent vaccine, recombinant HPV quadrivalent vaccine, recombinant interferon alfa-2b, rituximab, rituximab and hyaluronidase human, romiplostim, ropeginterferon alfa-2b-njft, sacituzumab govitecan-hziy, siltuximab, sintilimab, sipuleucel-T, tafasitamab-cxix, tagraxofusp-erzs, talimogene laherparepvec, tebentafusp-tebn, teclistamab-cqyv, tisagenlecleucel, tislelizumab, tisotumab vedotin-tftv, tocilizumab, toripalimab, trastuzumab, trastuzumab and hyaluronidase-oysk, tremelimumab-actl, ziv-aflibercept, and any combination thereof. One skilled in the art would immediately envisage an appropriate “conventional immunotherapy” based on the particular intestinal adenoma disclosed, as well as characteristics of an intestinal adenoma subject, such as, for example, the subject's sex, age, stage of the intestinal adenoma, genotype of the intestinal adenoma, and the like.

In certain embodiments, a NOS inhibitor composition, as described herein, may be administered as a suitable dosage form, as described herein, prior to, or alternatively, following a surgery to remove or resect an intestinal adenoma.

In some embodiments, a NOS inhibitor composition, as described herein, may be administered as a suitable dosage form, as described herein, to restore and/or maintain healthy and/or optimal expression levels of one or more antigens, as described herein, such as, for example, CD15, CD44, CD70, CD133, as well as any CD antigen that has an abnormal expression in an intestinal adenoma that would be readily envisaged by one skilled in the art, in a subject. In certain embodiments, the subject may have an intestinal adenoma or one or more symptoms thereof. As used herein, the terms “healthy” and “optimal” refer to expression levels of one or more antigens that would be typical of an equivalent subject that does not have an intestinal adenoma.

In certain embodiments, a NOS inhibitor composition, as described herein, may be administered as a suitable dosage form, as described herein, to restore and/or maintain healthy and/or optimal expression levels of one or more proteins, as described herein, such as, for example, ALDH1A3, mTOR, p-mTOR, TSC-2, AKT, pAKT, RPS6, p-RPS6, Syp, VEGF, PIGF, as well as any protein or enzyme that has an abnormal expression in an intestinal adenoma that would be readily envisaged by one skilled in the art, in a subject. In certain embodiments, the subject may have an intestinal adenoma or one or more symptoms thereof. As used herein, the terms “healthy” and “optimal” refer to expression levels of one or more proteins that would be typical of an equivalent subject that does not have an intestinal adenoma.

In some embodiments, a NOS inhibitor composition, as described herein, may be administered as a suitable dosage form, as described herein, to restore and/or maintain healthy and/or optimal expression levels of one or more NOS isoforms, as described herein, such as, for example, nNOS, iNOS, and eNOS, in a subject. In certain embodiments, the subject may have an intestinal adenoma or one or more symptoms thereof. As used herein, the terms “healthy” and “optimal” refer to expression levels of one or more NOS isoforms that would be typical of an equivalent subject that does not have an intestinal adenoma.

In certain embodiments, a NOS inhibitor composition, as described herein, may be administered as a suitable dosage form, as described herein, to restore and/or maintain healthy and/or optimal levels of one or more NO-indicators, as described herein, such as, for example, 3-Ntyr, NADPH-diaphorase, total nitrite concentration, total nitrate concentration, total nitrite and nitrate concentration, as well as any NO-indicator that has an elevated level in an intestinal adenoma that would be readily envisaged by one skilled in the art, in a subject. In certain embodiments, the subject may have an intestinal adenoma or one or more symptoms thereof. As used herein, the terms “healthy” and “optimal” refer to levels of one or more NO-indicators that would be typical of an equivalent subject that does not have an intestinal adenoma.

In certain embodiments, a NOS inhibitor composition, as described herein, may be administered as a suitable dosage form, as described herein, to treat, ameliorate, and/or reduce the severity of a sarcoma or one or more symptoms thereof in a subject (“sarcoma subject”). As used herein, the term “sarcoma” refers to a cancer that occurs and/or originates in a bone or connective tissue, such as a blood vessel, fat, lining of a joint, muscle, nerve, tendon, etc.

In some embodiments, a NOS inhibitor composition comprising 7-NI and/or a salt thereof may be administered as a suitable dosage form, as described herein, to treat, ameliorate, and/or reduce the severity of sarcoma or one or more symptoms thereof in a sarcoma subject. In certain embodiments, an nNOS inhibitor composition comprising 7-NI and/or a salt thereof may be administered to a sarcoma subject to treat, ameliorate, and/or reduce the severity of sarcoma or one or more symptoms thereof in the sarcoma subject: (i) via a route selected from the group consisting of buccally, inhalation, intraarterially, intraarticularly, intracardially, intracerebroventricularly, intradermally, intramedullary, intramuscularly, intraperitoneally, intrathecally, intravenously, intraventricularly, nasally, ophthalmically, orally, rectally, sublabially, sublingually, subcutaneously, topically, and transdermally; and/or (ii) with a frequency selected from the group consisting of four times daily, three times daily, twice daily, once daily, once every two days, once every three days, once every four days, once every five days, once every six days, once a week, once every eight days, once every nine days, once every ten days, once every two weeks, once every three weeks, and once a month; and/or (iii) at a dosage (based on subject weight) selected from the group consisting of 0.1 μg/kg, 0.5 μg/kg, 1 μg/kg, 1.5 μg/kg, 2.0 μg/kg, 2.5 μg/kg, 3.0 μg/kg, 3.5 μg/kg, 4.0 μg/kg, 4.5 μg/kg, 5.0 μg/kg, 10 μg/kg, 15 μg/kg, 20 μg/kg, 25 μg/kg, 30 μg/kg, 35 μg/kg, 40 μg/kg, 45 μg/kg, 50 μg/kg, 55 μg/kg, 60 μg/kg, 65 μg/kg, 70 μg/kg, 75 μg/kg, 80 μg/kg, 85 μg/kg, 90 μg/kg, 95 μg/kg, 100 μg/kg, 150 μg/kg, 200 μg/kg, 250 μg/kg, 300 μg/kg, 350 μg/kg, 400 μg/kg, 450 μg/kg, 500 μg/kg, 550 μg/kg, 600 μg/kg, 650 μg/kg, 700 μg/kg, 750 μg/kg, 800 μg/kg, 850 μg/kg, 900 μg/kg, 1 mg/kg, 1.5 mg/kg, 2.0 mg/kg, 2.5 mg/kg, 3 mg/kg, 3.5 mg/kg, 4.0 mg/kg, 4.5 mg/kg, 5 mg/kg, 5.5 mg/kg, 6 mg/kg, 6.5 mg/kg, 7 mg/kg, 7.5 mg/kg, 8 mg/kg, 8.5 mg/kg, 9 mg/kg, 9.5 mg/kg, 10 mg/kg 10.5 mg/kg, 11 mg/kg, 11.5 mg/kg, 12 mg/kg, 12.5 mg/kg, 13 mg/kg, 13.5 mg/kg, 14 mg/kg, 14.5 mg/kg, 15 mg/kg, 16 mg/kg, 17 mg/kg, 18 mg/kg, 19 mg/kg, 20 mg/kg, 21 mg/kg, 22 mg/kg, 23 mg/kg, 24 mg/kg, 25 mg/kg, 30 mg/kg, 35 mg/kg, 40 mg/kg, 45 mg/kg, 50 mg/kg, 55 mg/kg, 60 mg/kg, 65 mg/kg, 70 mg/kg, 75 mg/kg, 80 mg/kg, 85 mg/kg, 90 mg/kg, 95 mg/kg, 100 mg/kg, 105 mg/kg, 110 mg/kg, 115 mg/kg, 120 mg/kg, 125 mg/kg, 130 mg/kg, 135 mg/kg, 140 mg/kg, 145 mg/kg, 150 mg/kg, 155 mg/kg, 160 mg/kg, 165 mg/kg, 170 mg/kg, 175 mg/kg, 180 mg/kg, 185 mg/kg, 190 mg/kg, 195 mg/kg, 200 mg/kg, and any fraction or integer in between any two of the preceding amounts.

In certain embodiments, a NOS inhibitor composition comprising 3-Br-7-NI and/or a salt thereof may be administered as a suitable dosage form, as described herein, to treat, ameliorate, and/or reduce the severity of sarcoma or one or more symptoms thereof in a sarcoma subject. In some embodiments, an nNOS inhibitor composition comprising 3-Br-7-NI and/or a salt thereof may be administered to a sarcoma subject to treat, ameliorate, and/or reduce the severity of sarcoma or one or more symptoms thereof in the sarcoma subject: (i) via a route selected from the group consisting of buccally, inhalation, intraarterially, intraarticularly, intracardially, intracerebroventricularly, intradermally, intramedullary, intramuscularly, intraperitoneally, intrathecally, intravenously, intraventricularly, nasally, ophthalmically, orally, rectally, sublabially, sublingually, subcutaneously, topically, and transdermally; and/or (ii) with a frequency selected from the group consisting of four times daily, three times daily, twice daily, once daily, once every two days, once every three days, once every four days, once every five days, once every six days, once a week, once every eight days, once every nine days, once every ten days, once every two weeks, once every three weeks, and once a month; and/or (iii) at a dosage (based on subject weight) selected from the group consisting of 0.1 μg/kg, 0.5 μg/kg, 1 μg/kg, 1.5 μg/kg, 2.0 μg/kg, 2.5 μg/kg, 3.0 μg/kg, 3.5 μg/kg, 4.0 μg/kg, 4.5 μg/kg, 5.0 μg/kg, 10 μg/kg, 15 μg/kg, 20 μg/kg, 25 μg/kg, 30 μg/kg, 35 μg/kg, 40 μg/kg, 45 μg/kg, 50 μg/kg, 55 μg/kg, 60 μg/kg, 65 μg/kg, 70 μg/kg, 75 μg/kg, 80 μg/kg, 85 μg/kg, 90 μg/kg, 95 μg/kg, 100 μg/kg, 150 μg/kg, 200 μg/kg, 250 μg/kg, 300 μg/kg, 350 μg/kg, 400 μg/kg, 450 μg/kg, 500 μg/kg, 550 μg/kg, 600 μg/kg, 650 μg/kg, 700 μg/kg, 750 μg/kg, 800 μg/kg, 850 μg/kg, 900 μg/kg, 1 mg/kg, 1.5 mg/kg, 2.0 mg/kg, 2.5 mg/kg, 3 mg/kg, 3.5 mg/kg, 4.0 mg/kg, 4.5 mg/kg, 5 mg/kg, 5.5 mg/kg, 6 mg/kg, 6.5 mg/kg, 7 mg/kg, 7.5 mg/kg, 8 mg/kg, 8.5 mg/kg, 9 mg/kg, 9.5 mg/kg, 10 mg/kg 10.5 mg/kg, 11 mg/kg, 11.5 mg/kg, 12 mg/kg, 12.5 mg/kg, 13 mg/kg, 13.5 mg/kg, 14 mg/kg, 14.5 mg/kg, 15 mg/kg, 16 mg/kg, 17 mg/kg, 18 mg/kg, 19 mg/kg, 20 mg/kg, 21 mg/kg, 22 mg/kg, 23 mg/kg, 24 mg/kg, 25 mg/kg, 30 mg/kg, 35 mg/kg, 40 mg/kg, 45 mg/kg, 50 mg/kg, 55 mg/kg, 60 mg/kg, 65 mg/kg, 70 mg/kg, 75 mg/kg, 80 mg/kg, 85 mg/kg, 90 mg/kg, 95 mg/kg, 100 mg/kg, 105 mg/kg, 110 mg/kg, 115 mg/kg, 120 mg/kg, 125 mg/kg, 130 mg/kg, 135 mg/kg, 140 mg/kg, 145 mg/kg, 150 mg/kg, 155 mg/kg, 160 mg/kg, 165 mg/kg, 170 mg/kg, 175 mg/kg, 180 mg/kg, 185 mg/kg, 190 mg/kg, 195 mg/kg, 200 mg/kg, and any fraction or integer in between any two of the preceding amounts.

In some embodiments, a NOS inhibitor composition comprising L-NPA and/or a salt thereof, such as, for example, L-NPA hydrochloride, may be administered as a suitable dosage form, as described herein, to treat, ameliorate, and/or reduce the severity of sarcoma or one or more symptoms thereof in a sarcoma subject. In certain embodiments, an nNOS inhibitor composition comprising L-NPA and/or a salt thereof, such as, for example, L-NPA hydrochloride may be administered to a sarcoma subject to treat, ameliorate, and/or reduce the severity of sarcoma or one or more symptoms thereof in the sarcoma subject: (i) via a route selected from the group consisting of buccally, inhalation, intraarterially, intraarticularly, intracardially, intracerebroventricularly, intradermally, intramedullary, intramuscularly, intraperitoneally, intrathecally, intravenously, intraventricularly, nasally, ophthalmically, orally, rectally, sublabially, sublingually, subcutaneously, topically, and transdermally; and/or (ii) with a frequency selected from the group consisting of four times daily, three times daily, twice daily, once daily, once every two days, once every three days, once every four days, once every five days, once every six days, once a week, once every eight days, once every nine days, once every ten days, once every two weeks, once every three weeks, and once a month; and/or (iii) at a dosage (based on subject weight) selected from the group consisting of 0.1 μg/kg, 0.5 μg/kg, 1 μg/kg, 1.5 μg/kg, 2.0 μg/kg, 2.5 μg/kg, 3.0 μg/kg, 3.5 μg/kg, 4.0 μg/kg, 4.5 μg/kg, 5.0 μg/kg, 10 μg/kg, 15 μg/kg, 20 μg/kg, 25 μg/kg, 30 μg/kg, 35 μg/kg, 40 μg/kg, 45 μg/kg, 50 μg/kg, 55 μg/kg, 60 μg/kg, 65 μg/kg, 70 μg/kg, 75 μg/kg, 80 μg/kg, 85 μg/kg, 90 μg/kg, 95 μg/kg, 100 μg/kg, 150 μg/kg, 200 μg/kg, 250 μg/kg, 300 μg/kg, 350 μg/kg, 400 μg/kg, 450 μg/kg, 500 μg/kg, 550 μg/kg, 600 μg/kg, 650 μg/kg, 700 μg/kg, 750 μg/kg, 800 μg/kg, 850 μg/kg, 900 μg/kg, 1 mg/kg, 1.5 mg/kg, 2.0 mg/kg, 2.5 mg/kg, 3 mg/kg, 3.5 mg/kg, 4.0 mg/kg, 4.5 mg/kg, 5 mg/kg, 5.5 mg/kg, 6 mg/kg, 6.5 mg/kg, 7 mg/kg, 7.5 mg/kg, 8 mg/kg, 8.5 mg/kg, 9 mg/kg, 9.5 mg/kg, 10 mg/kg 10.5 mg/kg, 11 mg/kg, 11.5 mg/kg, 12 mg/kg, 12.5 mg/kg, 13 mg/kg, 13.5 mg/kg, 14 mg/kg, 14.5 mg/kg, 15 mg/kg, 16 mg/kg, 17 mg/kg, 18 mg/kg, 19 mg/kg, 20 mg/kg, 21 mg/kg, 22 mg/kg, 23 mg/kg, 24 mg/kg, 25 mg/kg, 30 mg/kg, 35 mg/kg, 40 mg/kg, 45 mg/kg, 50 mg/kg, 55 mg/kg, 60 mg/kg, 65 mg/kg, 70 mg/kg, 75 mg/kg, 80 mg/kg, 85 mg/kg, 90 mg/kg, 95 mg/kg, 100 mg/kg, 105 mg/kg, 110 mg/kg, 115 mg/kg, 120 mg/kg, 125 mg/kg, 130 mg/kg, 135 mg/kg, 140 mg/kg, 145 mg/kg, 150 mg/kg, 155 mg/kg, 160 mg/kg, 165 mg/kg, 170 mg/kg, 175 mg/kg, 180 mg/kg, 185 mg/kg, 190 mg/kg, 195 mg/kg, 200 mg/kg, and any fraction or integer in between any two of the preceding amounts.

In certain embodiments, a NOS inhibitor composition comprising L-NIO and/or a salt thereof, such as, for example, L-NIO dihydrochloride, may be administered as a suitable dosage form, as described herein, to treat, ameliorate, and/or reduce the severity of sarcoma or one or more symptoms thereof in a sarcoma subject. In some embodiments, an nNOS inhibitor composition comprising L-NIO and/or a salt thereof, such as, for example, L-NIO dihydrochloride, may be administered to a sarcoma subject to treat, ameliorate, and/or reduce the severity of sarcoma or one or more symptoms thereof in the sarcoma subject: (i) via a route selected from the group consisting of buccally, inhalation, intraarterially, intraarticularly, intracardially, intracerebroventricularly, intradermally, intramedullary, intramuscularly, intraperitoneally, intrathecally, intravenously, intraventricularly, nasally, ophthalmically, orally, rectally, sublabially, sublingually, subcutaneously, topically, and transdermally; and/or (ii) with a frequency selected from the group consisting of four times daily, three times daily, twice daily, once daily, once every two days, once every three days, once every four days, once every five days, once every six days, once a week, once every eight days, once every nine days, once every ten days, once every two weeks, once every three weeks, and once a month; and/or (iii) at a dosage (based on subject weight) selected from the group consisting of 0.1 μg/kg, 0.5 μg/kg, 1 μg/kg, 1.5 μg/kg, 2.0 μg/kg, 2.5 μg/kg, 3.0 μg/kg, 3.5 μg/kg, 4.0 μg/kg, 4.5 μg/kg, 5.0 μg/kg, 10 μg/kg, 15 μg/kg, 20 μg/kg, 25 μg/kg, 30 μg/kg, 35 μg/kg, 40 μg/kg, 45 μg/kg, 50 μg/kg, 55 μg/kg, 60 μg/kg, 65 μg/kg, 70 μg/kg, 75 μg/kg, 80 μg/kg, 85 μg/kg, 90 μg/kg, 95 μg/kg, 100 μg/kg, 150 μg/kg, 200 μg/kg, 250 μg/kg, 300 μg/kg, 350 μg/kg, 400 μg/kg, 450 μg/kg, 500 μg/kg, 550 μg/kg, 600 μg/kg, 650 μg/kg, 700 μg/kg, 750 μg/kg, 800 μg/kg, 850 μg/kg, 900 μg/kg, 1 mg/kg, 1.5 mg/kg, 2.0 mg/kg, 2.5 mg/kg, 3 mg/kg, 3.5 mg/kg, 4.0 mg/kg, 4.5 mg/kg, 5 mg/kg, 5.5 mg/kg, 6 mg/kg, 6.5 mg/kg, 7 mg/kg, 7.5 mg/kg, 8 mg/kg, 8.5 mg/kg, 9 mg/kg, 9.5 mg/kg, 10 mg/kg 10.5 mg/kg, 11 mg/kg, 11.5 mg/kg, 12 mg/kg, 12.5 mg/kg, 13 mg/kg, 13.5 mg/kg, 14 mg/kg, 14.5 mg/kg, 15 mg/kg, 16 mg/kg, 17 mg/kg, 18 mg/kg, 19 mg/kg, 20 mg/kg, 21 mg/kg, 22 mg/kg, 23 mg/kg, 24 mg/kg, 25 mg/kg, 30 mg/kg, 35 mg/kg, 40 mg/kg, 45 mg/kg, 50 mg/kg, 55 mg/kg, 60 mg/kg, 65 mg/kg, 70 mg/kg, 75 mg/kg, 80 mg/kg, 85 mg/kg, 90 mg/kg, 95 mg/kg, 100 mg/kg, 105 mg/kg, 110 mg/kg, 115 mg/kg, 120 mg/kg, 125 mg/kg, 130 mg/kg, 135 mg/kg, 140 mg/kg, 145 mg/kg, 150 mg/kg, 155 mg/kg, 160 mg/kg, 165 mg/kg, 170 mg/kg, 175 mg/kg, 180 mg/kg, 185 mg/kg, 190 mg/kg, 195 mg/kg, 200 mg/kg, and any fraction or integer in between any two of the preceding amounts.

In some embodiments, a NOS inhibitor composition comprising ARL-17477 and/or a salt thereof, such as, for example, ARL-17477 dihydrochloride, may be administered as a suitable dosage form, as described herein, to treat, ameliorate, and/or reduce the severity of sarcoma or one or more symptoms thereof in a sarcoma subject. In certain embodiments, an nNOS inhibitor composition comprising ARL-17477 and/or a salt thereof, such as, for example, ARL-17477 dihydrochloride, may be administered to a sarcoma subject to treat, ameliorate, and/or reduce the severity of sarcoma or one or more symptoms thereof in the sarcoma subject: (i) via a route selected from the group consisting of buccally, inhalation, intraarterially, intraarticularly, intracardially, intracerebroventricularly, intradermally, intramedullary, intramuscularly, intraperitoneally, intrathecally, intravenously, intraventricularly, nasally, ophthalmically, orally, rectally, sublabially, sublingually, subcutaneously, topically, and transdermally; and/or (ii) with a frequency selected from the group consisting of four times daily, three times daily, twice daily, once daily, once every two days, once every three days, once every four days, once every five days, once every six days, once a week, once every eight days, once every nine days, once every ten days, once every two weeks, once every three weeks, and once a month; and/or (iii) at a dosage (based on subject weight) selected from the group consisting of 0.1 μg/kg, 0.5 μg/kg, 1 μg/kg, 1.5 μg/kg, 2.0 μg/kg, 2.5 μg/kg, 3.0 μg/kg, 3.5 μg/kg, 4.0 μg/kg, 4.5 μg/kg, 5.0 μg/kg, 10 μg/kg, 15 μg/kg, 20 μg/kg, 25 μg/kg, 30 μg/kg, 35 μg/kg, 40 μg/kg, 45 μg/kg, 50 μg/kg, 55 μg/kg, 60 μg/kg, 65 μg/kg, 70 μg/kg, 75 μg/kg, 80 μg/kg, 85 μg/kg, 90 μg/kg, 95 μg/kg, 100 μg/kg, 150 μg/kg, 200 μg/kg, 250 μg/kg, 300 μg/kg, 350 μg/kg, 400 μg/kg, 450 μg/kg, 500 μg/kg, 550 μg/kg, 600 μg/kg, 650 μg/kg, 700 μg/kg, 750 μg/kg, 800 μg/kg, 850 μg/kg, 900 μg/kg, 1 mg/kg, 1.5 mg/kg, 2.0 mg/kg, 2.5 mg/kg, 3 mg/kg, 3.5 mg/kg, 4.0 mg/kg, 4.5 mg/kg, 5 mg/kg, 5.5 mg/kg, 6 mg/kg, 6.5 mg/kg, 7 mg/kg, 7.5 mg/kg, 8 mg/kg, 8.5 mg/kg, 9 mg/kg, 9.5 mg/kg, 10 mg/kg 10.5 mg/kg, 11 mg/kg, 11.5 mg/kg, 12 mg/kg, 12.5 mg/kg, 13 mg/kg, 13.5 mg/kg, 14 mg/kg, 14.5 mg/kg, 15 mg/kg, 16 mg/kg, 17 mg/kg, 18 mg/kg, 19 mg/kg, 20 mg/kg, 21 mg/kg, 22 mg/kg, 23 mg/kg, 24 mg/kg, 25 mg/kg, 30 mg/kg, 35 mg/kg, 40 mg/kg, 45 mg/kg, 50 mg/kg, 55 mg/kg, 60 mg/kg, 65 mg/kg, 70 mg/kg, 75 mg/kg, 80 mg/kg, 85 mg/kg, 90 mg/kg, 95 mg/kg, 100 mg/kg, 105 mg/kg, 110 mg/kg, 115 mg/kg, 120 mg/kg, 125 mg/kg, 130 mg/kg, 135 mg/kg, 140 mg/kg, 145 mg/kg, 150 mg/kg, 155 mg/kg, 160 mg/kg, 165 mg/kg, 170 mg/kg, 175 mg/kg, 180 mg/kg, 185 mg/kg, 190 mg/kg, 195 mg/kg, 200 mg/kg, and any fraction or integer in between any two of the preceding amounts.

In certain embodiments, a NOS inhibitor composition comprising L-NNA and/or a salt thereof may be administered as a suitable dosage form, as described herein, to treat, ameliorate, and/or reduce the severity of sarcoma or one or more symptoms thereof in a sarcoma subject. In some embodiments, an nNOS inhibitor composition comprising L-NNA and/or a salt thereof may be administered to a sarcoma subject to treat, ameliorate, and/or reduce the severity of sarcoma or one or more symptoms thereof in the sarcoma subject: (i) via a route selected from the group consisting of buccally, inhalation, intraarterially, intraarticularly, intracardially, intracerebroventricularly, intradermally, intramedullary, intramuscularly, intraperitoneally, intrathecally, intravenously, intraventricularly, nasally, ophthalmically, orally, rectally, sublabially, sublingually, subcutaneously, topically, and transdermally; and/or (ii) with a frequency selected from the group consisting of four times daily, three times daily, twice daily, once daily, once every two days, once every three days, once every four days, once every five days, once every six days, once a week, once every eight days, once every nine days, once every ten days, once every two weeks, once every three weeks, and once a month; and/or (iii) at a dosage (based on subject weight) selected from the group consisting of 0.1 μg/kg, 0.5 μg/kg, 1 μg/kg, 1.5 μg/kg, 2.0 μg/kg, 2.5 μg/kg, 3.0 μg/kg, 3.5 μg/kg, 4.0 μg/kg, 4.5 μg/kg, 5.0 μg/kg, 10 μg/kg, 15 μg/kg, 20 μg/kg, 25 μg/kg, 30 μg/kg, 35 μg/kg, 40 μg/kg, 45 μg/kg, 50 μg/kg, 55 μg/kg, 60 μg/kg, 65 μg/kg, 70 μg/kg, 75 μg/kg, 80 μg/kg, 85 μg/kg, 90 μg/kg, 95 μg/kg, 100 μg/kg, 150 μg/kg, 200 μg/kg, 250 μg/kg, 300 μg/kg, 350 μg/kg, 400 μg/kg, 450 μg/kg, 500 μg/kg, 550 μg/kg, 600 μg/kg, 650 μg/kg, 700 μg/kg, 750 μg/kg, 800 μg/kg, 850 μg/kg, 900 μg/kg, 1 mg/kg, 1.5 mg/kg, 2.0 mg/kg, 2.5 mg/kg, 3 mg/kg, 3.5 mg/kg, 4.0 mg/kg, 4.5 mg/kg, 5 mg/kg, 5.5 mg/kg, 6 mg/kg, 6.5 mg/kg, 7 mg/kg, 7.5 mg/kg, 8 mg/kg, 8.5 mg/kg, 9 mg/kg, 9.5 mg/kg, 10 mg/kg 10.5 mg/kg, 11 mg/kg, 11.5 mg/kg, 12 mg/kg, 12.5 mg/kg, 13 mg/kg, 13.5 mg/kg, 14 mg/kg, 14.5 mg/kg, 15 mg/kg, 16 mg/kg, 17 mg/kg, 18 mg/kg, 19 mg/kg, 20 mg/kg, 21 mg/kg, 22 mg/kg, 23 mg/kg, 24 mg/kg, 25 mg/kg, 30 mg/kg, 35 mg/kg, 40 mg/kg, 45 mg/kg, 50 mg/kg, 55 mg/kg, 60 mg/kg, 65 mg/kg, 70 mg/kg, 75 mg/kg, 80 mg/kg, 85 mg/kg, 90 mg/kg, 95 mg/kg, 100 mg/kg, 105 mg/kg, 110 mg/kg, 115 mg/kg, 120 mg/kg, 125 mg/kg, 130 mg/kg, 135 mg/kg, 140 mg/kg, 145 mg/kg, 150 mg/kg, 155 mg/kg, 160 mg/kg, 165 mg/kg, 170 mg/kg, 175 mg/kg, 180 mg/kg, 185 mg/kg, 190 mg/kg, 195 mg/kg, 200 mg/kg, and any fraction or integer in between any two of the preceding amounts.

In some embodiments, a NOS inhibitor composition comprising L-NAME and/or a salt thereof, such as, for example, L-NAME hydrochloride, may be administered as a suitable dosage form, as described herein, to treat, ameliorate, and/or reduce the severity of sarcoma or one or more symptoms thereof in a sarcoma subject. In certain embodiments, an nNOS inhibitor composition comprising L-NAME and/or a salt thereof, such as, for example, L-NAME hydrochloride, may be administered to a sarcoma subject to treat, ameliorate, and/or reduce the severity of sarcoma or one or more symptoms thereof in the sarcoma subject: (i) via a route selected from the group consisting of buccally, inhalation, intraarterially, intraarticularly, intracardially, intracerebroventricularly, intradermally, intramedullary, intramuscularly, intraperitoneally, intrathecally, intravenously, intraventricularly, nasally, ophthalmically, orally, rectally, sublabially, sublingually, subcutaneously, topically, and transdermally; and/or (ii) with a frequency selected from the group consisting of four times daily, three times daily, twice daily, once daily, once every two days, once every three days, once every four days, once every five days, once every six days, once a week, once every eight days, once every nine days, once every ten days, once every two weeks, once every three weeks, and once a month; and/or (iii) at a dosage (based on subject weight) selected from the group consisting of 0.1 μg/kg, 0.5 μg/kg, 1 μg/kg, 1.5 μg/kg, 2.0 μg/kg, 2.5 μg/kg, 3.0 μg/kg, 3.5 μg/kg, 4.0 μg/kg, 4.5 μg/kg, 5.0 μg/kg, 10 μg/kg, 15 μg/kg, 20 μg/kg, 25 μg/kg, 30 μg/kg, 35 μg/kg, 40 μg/kg, 45 μg/kg, 50 μg/kg, 55 μg/kg, 60 μg/kg, 65 μg/kg, 70 μg/kg, 75 μg/kg, 80 μg/kg, 85 μg/kg, 90 μg/kg, 95 μg/kg, 100 μg/kg, 150 μg/kg, 200 μg/kg, 250 μg/kg, 300 μg/kg, 350 μg/kg, 400 μg/kg, 450 μg/kg, 500 μg/kg, 550 μg/kg, 600 μg/kg, 650 μg/kg, 700 μg/kg, 750 μg/kg, 800 μg/kg, 850 μg/kg, 900 μg/kg, 1 mg/kg, 1.5 mg/kg, 2.0 mg/kg, 2.5 mg/kg, 3 mg/kg, 3.5 mg/kg, 4.0 mg/kg, 4.5 mg/kg, 5 mg/kg, 5.5 mg/kg, 6 mg/kg, 6.5 mg/kg, 7 mg/kg, 7.5 mg/kg, 8 mg/kg, 8.5 mg/kg, 9 mg/kg, 9.5 mg/kg, 10 mg/kg 10.5 mg/kg, 11 mg/kg, 11.5 mg/kg, 12 mg/kg, 12.5 mg/kg, 13 mg/kg, 13.5 mg/kg, 14 mg/kg, 14.5 mg/kg, 15 mg/kg, 16 mg/kg, 17 mg/kg, 18 mg/kg, 19 mg/kg, 20 mg/kg, 21 mg/kg, 22 mg/kg, 23 mg/kg, 24 mg/kg, 25 mg/kg, 30 mg/kg, 35 mg/kg, 40 mg/kg, 45 mg/kg, 50 mg/kg, 55 mg/kg, 60 mg/kg, 65 mg/kg, 70 mg/kg, 75 mg/kg, 80 mg/kg, 85 mg/kg, 90 mg/kg, 95 mg/kg, 100 mg/kg, 105 mg/kg, 110 mg/kg, 115 mg/kg, 120 mg/kg, 125 mg/kg, 130 mg/kg, 135 mg/kg, 140 mg/kg, 145 mg/kg, 150 mg/kg, 155 mg/kg, 160 mg/kg, 165 mg/kg, 170 mg/kg, 175 mg/kg, 180 mg/kg, 185 mg/kg, 190 mg/kg, 195 mg/kg, 200 mg/kg, and any fraction or integer in between any two of the preceding amounts.

In certain embodiments, a NOS inhibitor composition comprising L-NMMA and/or a salt thereof, such as, for example, L-NMMA acetate, may be administered as a suitable dosage form, as described herein, to treat, ameliorate, and/or reduce the severity of sarcoma or one or more symptoms thereof in a sarcoma subject. In some embodiments, an nNOS inhibitor composition comprising L-NMMA and/or a salt thereof, such as, for example, L-NMMA acetate, may be administered to a sarcoma subject to treat, ameliorate, and/or reduce the severity of sarcoma or one or more symptoms thereof in the sarcoma subject: (i) via a route selected from the group consisting of buccally, inhalation, intraarterially, intraarticularly, intracardially, intracerebroventricularly, intradermally, intramedullary, intramuscularly, intraperitoneally, intrathecally, intravenously, intraventricularly, nasally, ophthalmically, orally, rectally, sublabially, sublingually, subcutaneously, topically, and transdermally; and/or (ii) with a frequency selected from the group consisting of four times daily, three times daily, twice daily, once daily, once every two days, once every three days, once every four days, once every five days, once every six days, once a week, once every eight days, once every nine days, once every ten days, once every two weeks, once every three weeks, and once a month; and/or (iii) at a dosage (based on subject weight) selected from the group consisting of 0.1 μg/kg, 0.5 μg/kg, 1 μg/kg, 1.5 μg/kg, 2.0 μg/kg, 2.5 μg/kg, 3.0 μg/kg, 3.5 μg/kg, 4.0 μg/kg, 4.5 μg/kg, 5.0 μg/kg, 10 μg/kg, 15 μg/kg, 20 μg/kg, 25 μg/kg, 30 μg/kg, 35 μg/kg, 40 μg/kg, 45 μg/kg, 50 μg/kg, 55 μg/kg, 60 μg/kg, 65 μg/kg, 70 μg/kg, 75 μg/kg, 80 μg/kg, 85 μg/kg, 90 μg/kg, 95 μg/kg, 100 μg/kg, 150 μg/kg, 200 μg/kg, 250 μg/kg, 300 μg/kg, 350 μg/kg, 400 μg/kg, 450 μg/kg, 500 μg/kg, 550 μg/kg, 600 μg/kg, 650 μg/kg, 700 μg/kg, 750 μg/kg, 800 μg/kg, 850 μg/kg, 900 μg/kg, 1 mg/kg, 1.5 mg/kg, 2.0 mg/kg, 2.5 mg/kg, 3 mg/kg, 3.5 mg/kg, 4.0 mg/kg, 4.5 mg/kg, 5 mg/kg, 5.5 mg/kg, 6 mg/kg, 6.5 mg/kg, 7 mg/kg, 7.5 mg/kg, 8 mg/kg, 8.5 mg/kg, 9 mg/kg, 9.5 mg/kg, 10 mg/kg 10.5 mg/kg, 11 mg/kg, 11.5 mg/kg, 12 mg/kg, 12.5 mg/kg, 13 mg/kg, 13.5 mg/kg, 14 mg/kg, 14.5 mg/kg, 15 mg/kg, 16 mg/kg, 17 mg/kg, 18 mg/kg, 19 mg/kg, 20 mg/kg, 21 mg/kg, 22 mg/kg, 23 mg/kg, 24 mg/kg, 25 mg/kg, 30 mg/kg, 35 mg/kg, 40 mg/kg, 45 mg/kg, 50 mg/kg, 55 mg/kg, 60 mg/kg, 65 mg/kg, 70 mg/kg, 75 mg/kg, 80 mg/kg, 85 mg/kg, 90 mg/kg, 95 mg/kg, 100 mg/kg, 105 mg/kg, 110 mg/kg, 115 mg/kg, 120 mg/kg, 125 mg/kg, 130 mg/kg, 135 mg/kg, 140 mg/kg, 145 mg/kg, 150 mg/kg, 155 mg/kg, 160 mg/kg, 165 mg/kg, 170 mg/kg, 175 mg/kg, 180 mg/kg, 185 mg/kg, 190 mg/kg, 195 mg/kg, 200 mg/kg, and any fraction or integer in between any two of the preceding amounts.

In some embodiments, a NOS inhibitor composition comprising GW-274150 and/or a salt thereof, such as, for example, GW-274150 phosphate, may be administered as a suitable dosage form, as described herein, to treat, ameliorate, and/or reduce the severity of sarcoma or one or more symptoms thereof in a sarcoma subject. In certain embodiments, an nNOS inhibitor composition comprising GW-274150 and/or a salt thereof, such as, for example, GW-274150 phosphate, may be administered to a sarcoma subject to treat, ameliorate, and/or reduce the severity of sarcoma or one or more symptoms thereof in the sarcoma subject: (i) via a route selected from the group consisting of buccally, inhalation, intraarterially, intraarticularly, intracardially, intracerebroventricularly, intradermally, intramedullary, intramuscularly, intraperitoneally, intrathecally, intravenously, intraventricularly, nasally, ophthalmically, orally, rectally, sublabially, sublingually, subcutaneously, topically, and transdermally; and/or (ii) with a frequency selected from the group consisting of four times daily, three times daily, twice daily, once daily, once every two days, once every three days, once every four days, once every five days, once every six days, once a week, once every eight days, once every nine days, once every ten days, once every two weeks, once every three weeks, and once a month; and/or (iii) at a dosage (based on subject weight) selected from the group consisting of 0.1 μg/kg, 0.5 μg/kg, 1 μg/kg, 1.5 μg/kg, 2.0 μg/kg, 2.5 μg/kg, 3.0 μg/kg, 3.5 μg/kg, 4.0 μg/kg, 4.5 μg/kg, 5.0 μg/kg, 10 μg/kg, 15 μg/kg, 20 μg/kg, 25 μg/kg, 30 μg/kg, 35 μg/kg, 40 μg/kg, 45 μg/kg, 50 μg/kg, 55 μg/kg, 60 μg/kg, 65 μg/kg, 70 μg/kg, 75 μg/kg, 80 μg/kg, 85 μg/kg, 90 μg/kg, 95 μg/kg, 100 μg/kg, 150 μg/kg, 200 μg/kg, 250 μg/kg, 300 μg/kg, 350 μg/kg, 400 μg/kg, 450 μg/kg, 500 μg/kg, 550 μg/kg, 600 μg/kg, 650 μg/kg, 700 μg/kg, 750 μg/kg, 800 μg/kg, 850 μg/kg, 900 μg/kg, 1 mg/kg, 1.5 mg/kg, 2.0 mg/kg, 2.5 mg/kg, 3 mg/kg, 3.5 mg/kg, 4.0 mg/kg, 4.5 mg/kg, 5 mg/kg, 5.5 mg/kg, 6 mg/kg, 6.5 mg/kg, 7 mg/kg, 7.5 mg/kg, 8 mg/kg, 8.5 mg/kg, 9 mg/kg, 9.5 mg/kg, 10 mg/kg 10.5 mg/kg, 11 mg/kg, 11.5 mg/kg, 12 mg/kg, 12.5 mg/kg, 13 mg/kg, 13.5 mg/kg, 14 mg/kg, 14.5 mg/kg, 15 mg/kg, 16 mg/kg, 17 mg/kg, 18 mg/kg, 19 mg/kg, 20 mg/kg, 21 mg/kg, 22 mg/kg, 23 mg/kg, 24 mg/kg, 25 mg/kg, 30 mg/kg, 35 mg/kg, 40 mg/kg, 45 mg/kg, 50 mg/kg, 55 mg/kg, 60 mg/kg, 65 mg/kg, 70 mg/kg, 75 mg/kg, 80 mg/kg, 85 mg/kg, 90 mg/kg, 95 mg/kg, 100 mg/kg, 105 mg/kg, 110 mg/kg, 115 mg/kg, 120 mg/kg, 125 mg/kg, 130 mg/kg, 135 mg/kg, 140 mg/kg, 145 mg/kg, 150 mg/kg, 155 mg/kg, 160 mg/kg, 165 mg/kg, 170 mg/kg, 175 mg/kg, 180 mg/kg, 185 mg/kg, 190 mg/kg, 195 mg/kg, 200 mg/kg, and any fraction or integer in between any two of the preceding amounts.

In certain embodiments, a NOS inhibitor composition comprising L-NIL and/or a salt thereof, such as, for example, L-NIL hydrochloride, may be administered as a suitable dosage form, as described herein, to treat, ameliorate, and/or reduce the severity of sarcoma or one or more symptoms thereof in a sarcoma subject. In some embodiments, an nNOS inhibitor composition comprising L-NIL and/or a salt thereof, such as, for example, L-NIL hydrochloride, may be administered to a sarcoma subject to treat, ameliorate, and/or reduce the severity of sarcoma or one or more symptoms thereof in the sarcoma subject: (i) via a route selected from the group consisting of buccally, inhalation, intraarterially, intraarticularly, intracardially, intracerebroventricularly, intradermally, intramedullary, intramuscularly, intraperitoneally, intrathecally, intravenously, intraventricularly, nasally, ophthalmically, orally, rectally, sublabially, sublingually, subcutaneously, topically, and transdermally; and/or (ii) with a frequency selected from the group consisting of four times daily, three times daily, twice daily, once daily, once every two days, once every three days, once every four days, once every five days, once every six days, once a week, once every eight days, once every nine days, once every ten days, once every two weeks, once every three weeks, and once a month; and/or (iii) at a dosage (based on subject weight) selected from the group consisting of 0.1 μg/kg, 0.5 μg/kg, 1 μg/kg, 1.5 μg/kg, 2.0 μg/kg, 2.5 μg/kg, 3.0 μg/kg, 3.5 μg/kg, 4.0 μg/kg, 4.5 μg/kg, 5.0 μg/kg, 10 μg/kg, 15 μg/kg, 20 μg/kg, 25 μg/kg, 30 μg/kg, 35 μg/kg, 40 μg/kg, 45 μg/kg, 50 μg/kg, 55 μg/kg, 60 μg/kg, 65 μg/kg, 70 μg/kg, 75 μg/kg, 80 μg/kg, 85 μg/kg, 90 μg/kg, 95 μg/kg, 100 μg/kg, 150 μg/kg, 200 μg/kg, 250 μg/kg, 300 μg/kg, 350 μg/kg, 400 μg/kg, 450 μg/kg, 500 μg/kg, 550 μg/kg, 600 μg/kg, 650 μg/kg, 700 μg/kg, 750 μg/kg, 800 μg/kg, 850 μg/kg, 900 μg/kg, 1 mg/kg, 1.5 mg/kg, 2.0 mg/kg, 2.5 mg/kg, 3 mg/kg, 3.5 mg/kg, 4.0 mg/kg, 4.5 mg/kg, 5 mg/kg, 5.5 mg/kg, 6 mg/kg, 6.5 mg/kg, 7 mg/kg, 7.5 mg/kg, 8 mg/kg, 8.5 mg/kg, 9 mg/kg, 9.5 mg/kg, 10 mg/kg 10.5 mg/kg, 11 mg/kg, 11.5 mg/kg, 12 mg/kg, 12.5 mg/kg, 13 mg/kg, 13.5 mg/kg, 14 mg/kg, 14.5 mg/kg, 15 mg/kg, 16 mg/kg, 17 mg/kg, 18 mg/kg, 19 mg/kg, 20 mg/kg, 21 mg/kg, 22 mg/kg, 23 mg/kg, 24 mg/kg, 25 mg/kg, 30 mg/kg, 35 mg/kg, 40 mg/kg, 45 mg/kg, 50 mg/kg, 55 mg/kg, 60 mg/kg, 65 mg/kg, 70 mg/kg, 75 mg/kg, 80 mg/kg, 85 mg/kg, 90 mg/kg, 95 mg/kg, 100 mg/kg, 105 mg/kg, 110 mg/kg, 115 mg/kg, 120 mg/kg, 125 mg/kg, 130 mg/kg, 135 mg/kg, 140 mg/kg, 145 mg/kg, 150 mg/kg, 155 mg/kg, 160 mg/kg, 165 mg/kg, 170 mg/kg, 175 mg/kg, 180 mg/kg, 185 mg/kg, 190 mg/kg, 195 mg/kg, 200 mg/kg, and any fraction or integer in between any two of the preceding amounts.

In some embodiments, a NOS inhibitor composition comprising 1400W and/or a salt thereof, such as, for example, 1400W dihydrochloride, may be administered as a suitable dosage form, as described herein, to treat, ameliorate, and/or reduce the severity of sarcoma or one or more symptoms thereof in a sarcoma subject. In certain embodiments, an nNOS inhibitor composition comprising 1400W and/or a salt thereof, such as, for example, 1400W dihydrochloride, may be administered to a sarcoma subject to treat, ameliorate, and/or reduce the severity of sarcoma or one or more symptoms thereof in the sarcoma subject: (i) via a route selected from the group consisting of buccally, inhalation, intraarterially, intraarticularly, intracardially, intracerebroventricularly, intradermally, intramedullary, intramuscularly, intraperitoneally, intrathecally, intravenously, intraventricularly, nasally, ophthalmically, orally, rectally, sublabially, sublingually, subcutaneously, topically, and transdermally; and/or (ii) with a frequency selected from the group consisting of four times daily, three times daily, twice daily, once daily, once every two days, once every three days, once every four days, once every five days, once every six days, once a week, once every eight days, once every nine days, once every ten days, once every two weeks, once every three weeks, and once a month; and/or (iii) at a dosage (based on subject weight) selected from the group consisting of 0.1 μg/kg, 0.5 μg/kg, 1 μg/kg, 1.5 μg/kg, 2.0 μg/kg, 2.5 μg/kg, 3.0 μg/kg, 3.5 μg/kg, 4.0 μg/kg, 4.5 μg/kg, 5.0 μg/kg, 10 μg/kg, 15 μg/kg, 20 μg/kg, 25 μg/kg, 30 μg/kg, 35 μg/kg, 40 μg/kg, 45 μg/kg, 50 μg/kg, 55 μg/kg, 60 μg/kg, 65 μg/kg, 70 μg/kg, 75 μg/kg, 80 μg/kg, 85 μg/kg, 90 μg/kg, 95 μg/kg, 100 μg/kg, 150 μg/kg, 200 μg/kg, 250 μg/kg, 300 μg/kg, 350 μg/kg, 400 μg/kg, 450 μg/kg, 500 μg/kg, 550 μg/kg, 600 μg/kg, 650 μg/kg, 700 μg/kg, 750 μg/kg, 800 μg/kg, 850 μg/kg, 900 μg/kg, 1 mg/kg, 1.5 mg/kg, 2.0 mg/kg, 2.5 mg/kg, 3 mg/kg, 3.5 mg/kg, 4.0 mg/kg, 4.5 mg/kg, 5 mg/kg, 5.5 mg/kg, 6 mg/kg, 6.5 mg/kg, 7 mg/kg, 7.5 mg/kg, 8 mg/kg, 8.5 mg/kg, 9 mg/kg, 9.5 mg/kg, 10 mg/kg 10.5 mg/kg, 11 mg/kg, 11.5 mg/kg, 12 mg/kg, 12.5 mg/kg, 13 mg/kg, 13.5 mg/kg, 14 mg/kg, 14.5 mg/kg, 15 mg/kg, 16 mg/kg, 17 mg/kg, 18 mg/kg, 19 mg/kg, 20 mg/kg, 21 mg/kg, 22 mg/kg, 23 mg/kg, 24 mg/kg, 25 mg/kg, 30 mg/kg, 35 mg/kg, 40 mg/kg, 45 mg/kg, 50 mg/kg, 55 mg/kg, 60 mg/kg, 65 mg/kg, 70 mg/kg, 75 mg/kg, 80 mg/kg, 85 mg/kg, 90 mg/kg, 95 mg/kg, 100 mg/kg, 105 mg/kg, 110 mg/kg, 115 mg/kg, 120 mg/kg, 125 mg/kg, 130 mg/kg, 135 mg/kg, 140 mg/kg, 145 mg/kg, 150 mg/kg, 155 mg/kg, 160 mg/kg, 165 mg/kg, 170 mg/kg, 175 mg/kg, 180 mg/kg, 185 mg/kg, 190 mg/kg, 195 mg/kg, 200 mg/kg, and any fraction or integer in between any two of the preceding amounts.

In certain embodiments, a NOS inhibitor composition comprising OKN-007 and/or a salt thereof, such as, for example, OKN-007 sodium, may be administered as a suitable dosage form, as described herein, to treat, ameliorate, and/or reduce the severity of sarcoma or one or more symptoms thereof in a sarcoma subject. In some embodiments, an nNOS inhibitor composition comprising OKN-007 and/or a salt thereof, such as, for example, OKN-007 sodium, may be administered to a sarcoma subject to treat, ameliorate, and/or reduce the severity of sarcoma or one or more symptoms thereof in the sarcoma subject: (i) via a route selected from the group consisting of buccally, inhalation, intraarterially, intraarticularly, intracardially, intracerebroventricularly, intradermally, intramedullary, intramuscularly, intraperitoneally, intrathecally, intravenously, intraventricularly, nasally, ophthalmically, orally, rectally, sublabially, sublingually, subcutaneously, topically, and transdermally; and/or (ii) with a frequency selected from the group consisting of four times daily, three times daily, twice daily, once daily, once every two days, once every three days, once every four days, once every five days, once every six days, once a week, once every eight days, once every nine days, once every ten days, once every two weeks, once every three weeks, and once a month; and/or (iii) at a dosage (based on subject weight) selected from the group consisting of 0.1 μg/kg, 0.5 μg/kg, 1 μg/kg, 1.5 μg/kg, 2.0 μg/kg, 2.5 μg/kg, 3.0 μg/kg, 3.5 μg/kg, 4.0 μg/kg, 4.5 μg/kg, 5.0 μg/kg, 10 μg/kg, 15 μg/kg, 20 μg/kg, 25 μg/kg, 30 μg/kg, 35 μg/kg, 40 μg/kg, 45 μg/kg, 50 μg/kg, 55 μg/kg, 60 μg/kg, 65 μg/kg, 70 μg/kg, 75 μg/kg, 80 μg/kg, 85 μg/kg, 90 μg/kg, 95 μg/kg, 100 μg/kg, 150 μg/kg, 200 μg/kg, 250 μg/kg, 300 μg/kg, 350 μg/kg, 400 μg/kg, 450 μg/kg, 500 μg/kg, 550 μg/kg, 600 μg/kg, 650 μg/kg, 700 μg/kg, 750 μg/kg, 800 μg/kg, 850 μg/kg, 900 μg/kg, 1 mg/kg, 1.5 mg/kg, 2.0 mg/kg, 2.5 mg/kg, 3 mg/kg, 3.5 mg/kg, 4.0 mg/kg, 4.5 mg/kg, 5 mg/kg, 5.5 mg/kg, 6 mg/kg, 6.5 mg/kg, 7 mg/kg, 7.5 mg/kg, 8 mg/kg, 8.5 mg/kg, 9 mg/kg, 9.5 mg/kg, 10 mg/kg 10.5 mg/kg, 11 mg/kg, 11.5 mg/kg, 12 mg/kg, 12.5 mg/kg, 13 mg/kg, 13.5 mg/kg, 14 mg/kg, 14.5 mg/kg, 15 mg/kg, 16 mg/kg, 17 mg/kg, 18 mg/kg, 19 mg/kg, 20 mg/kg, 21 mg/kg, 22 mg/kg, 23 mg/kg, 24 mg/kg, 25 mg/kg, 30 mg/kg, 35 mg/kg, 40 mg/kg, 45 mg/kg, 50 mg/kg, 55 mg/kg, 60 mg/kg, 65 mg/kg, 70 mg/kg, 75 mg/kg, 80 mg/kg, 85 mg/kg, 90 mg/kg, 95 mg/kg, 100 mg/kg, 105 mg/kg, 110 mg/kg, 115 mg/kg, 120 mg/kg, 125 mg/kg, 130 mg/kg, 135 mg/kg, 140 mg/kg, 145 mg/kg, 150 mg/kg, 155 mg/kg, 160 mg/kg, 165 mg/kg, 170 mg/kg, 175 mg/kg, 180 mg/kg, 185 mg/kg, 190 mg/kg, 195 mg/kg, 200 mg/kg, and any fraction or integer in between any two of the preceding amounts.

In some embodiments, a NOS inhibitor composition comprising BYK-191023 and/or a salt thereof, such as, for example, BYK-191023 dihydrochloride, may be administered as a suitable dosage form, as described herein, to treat, ameliorate, and/or reduce the severity of sarcoma or one or more symptoms thereof in a sarcoma subject. In certain embodiments, an nNOS inhibitor composition comprising BYK-191023 and/or a salt thereof, such as, for example, BYK-191023 dihydrochloride, may be administered to a sarcoma subject to treat, ameliorate, and/or reduce the severity of sarcoma or one or more symptoms thereof in the sarcoma subject: (i) via a route selected from the group consisting of buccally, inhalation, intraarterially, intraarticularly, intracardially, intracerebroventricularly, intradermally, intramedullary, intramuscularly, intraperitoneally, intrathecally, intravenously, intraventricularly, nasally, ophthalmically, orally, rectally, sublabially, sublingually, subcutaneously, topically, and transdermally; and/or (ii) with a frequency selected from the group consisting of four times daily, three times daily, twice daily, once daily, once every two days, once every three days, once every four days, once every five days, once every six days, once a week, once every eight days, once every nine days, once every ten days, once every two weeks, once every three weeks, and once a month; and/or (iii) at a dosage (based on subject weight) selected from the group consisting of 0.1 μg/kg, 0.5 μg/kg, 1 μg/kg, 1.5 μg/kg, 2.0 μg/kg, 2.5 μg/kg, 3.0 μg/kg, 3.5 μg/kg, 4.0 μg/kg, 4.5 μg/kg, 5.0 μg/kg, 10 μg/kg, 15 μg/kg, 20 μg/kg, 25 μg/kg, 30 μg/kg, 35 μg/kg, 40 μg/kg, 45 μg/kg, 50 μg/kg, 55 μg/kg, 60 μg/kg, 65 μg/kg, 70 μg/kg, 75 μg/kg, 80 μg/kg, 85 μg/kg, 90 μg/kg, 95 μg/kg, 100 μg/kg, 150 μg/kg, 200 μg/kg, 250 μg/kg, 300 μg/kg, 350 μg/kg, 400 μg/kg, 450 μg/kg, 500 μg/kg, 550 μg/kg, 600 μg/kg, 650 μg/kg, 700 μg/kg, 750 μg/kg, 800 μg/kg, 850 μg/kg, 900 μg/kg, 1 mg/kg, 1.5 mg/kg, 2.0 mg/kg, 2.5 mg/kg, 3 mg/kg, 3.5 mg/kg, 4.0 mg/kg, 4.5 mg/kg, 5 mg/kg, 5.5 mg/kg, 6 mg/kg, 6.5 mg/kg, 7 mg/kg, 7.5 mg/kg, 8 mg/kg, 8.5 mg/kg, 9 mg/kg, 9.5 mg/kg, 10 mg/kg 10.5 mg/kg, 11 mg/kg, 11.5 mg/kg, 12 mg/kg, 12.5 mg/kg, 13 mg/kg, 13.5 mg/kg, 14 mg/kg, 14.5 mg/kg, 15 mg/kg, 16 mg/kg, 17 mg/kg, 18 mg/kg, 19 mg/kg, 20 mg/kg, 21 mg/kg, 22 mg/kg, 23 mg/kg, 24 mg/kg, 25 mg/kg, 30 mg/kg, 35 mg/kg, 40 mg/kg, 45 mg/kg, 50 mg/kg, 55 mg/kg, 60 mg/kg, 65 mg/kg, 70 mg/kg, 75 mg/kg, 80 mg/kg, 85 mg/kg, 90 mg/kg, 95 mg/kg, 100 mg/kg, 105 mg/kg, 110 mg/kg, 115 mg/kg, 120 mg/kg, 125 mg/kg, 130 mg/kg, 135 mg/kg, 140 mg/kg, 145 mg/kg, 150 mg/kg, 155 mg/kg, 160 mg/kg, 165 mg/kg, 170 mg/kg, 175 mg/kg, 180 mg/kg, 185 mg/kg, 190 mg/kg, 195 mg/kg, 200 mg/kg, and any fraction or integer in between any two of the preceding amounts.

In certain embodiments, a NOS inhibitor composition comprising CM544 and/or a salt thereof, such as, for example, CM544 dihydrochloride, may be administered as a suitable dosage form, as described herein, to treat, ameliorate, and/or reduce the severity of sarcoma or one or more symptoms thereof in a sarcoma subject. In some embodiments, an nNOS inhibitor composition comprising CM544 and/or a salt thereof, such as, for example, CM544 dihydrochloride, may be administered to a sarcoma subject to treat, ameliorate, and/or reduce the severity of sarcoma or one or more symptoms thereof in the sarcoma subject: (i) via a route selected from the group consisting of buccally, inhalation, intraarterially, intraarticularly, intracardially, intracerebroventricularly, intradermally, intramedullary, intramuscularly, intraperitoneally, intrathecally, intravenously, intraventricularly, nasally, ophthalmically, orally, rectally, sublabially, sublingually, subcutaneously, topically, and transdermally; and/or (ii) with a frequency selected from the group consisting of four times daily, three times daily, twice daily, once daily, once every two days, once every three days, once every four days, once every five days, once every six days, once a week, once every eight days, once every nine days, once every ten days, once every two weeks, once every three weeks, and once a month; and/or (iii) at a dosage (based on subject weight) selected from the group consisting of 0.1 μg/kg, 0.5 μg/kg, 1 μg/kg, 1.5 μg/kg, 2.0 μg/kg, 2.5 μg/kg, 3.0 μg/kg, 3.5 μg/kg, 4.0 μg/kg, 4.5 μg/kg, 5.0 μg/kg, 10 μg/kg, 15 μg/kg, 20 μg/kg, 25 μg/kg, 30 μg/kg, 35 μg/kg, 40 μg/kg, 45 μg/kg, 50 μg/kg, 55 μg/kg, 60 μg/kg, 65 μg/kg, 70 μg/kg, 75 μg/kg, 80 μg/kg, 85 μg/kg, 90 μg/kg, 95 μg/kg, 100 μg/kg, 150 μg/kg, 200 μg/kg, 250 μg/kg, 300 μg/kg, 350 μg/kg, 400 μg/kg, 450 μg/kg, 500 μg/kg, 550 μg/kg, 600 μg/kg, 650 μg/kg, 700 μg/kg, 750 μg/kg, 800 μg/kg, 850 μg/kg, 900 μg/kg, 1 mg/kg, 1.5 mg/kg, 2.0 mg/kg, 2.5 mg/kg, 3 mg/kg, 3.5 mg/kg, 4.0 mg/kg, 4.5 mg/kg, 5 mg/kg, 5.5 mg/kg, 6 mg/kg, 6.5 mg/kg, 7 mg/kg, 7.5 mg/kg, 8 mg/kg, 8.5 mg/kg, 9 mg/kg, 9.5 mg/kg, 10 mg/kg 10.5 mg/kg, 11 mg/kg, 11.5 mg/kg, 12 mg/kg, 12.5 mg/kg, 13 mg/kg, 13.5 mg/kg, 14 mg/kg, 14.5 mg/kg, 15 mg/kg, 16 mg/kg, 17 mg/kg, 18 mg/kg, 19 mg/kg, 20 mg/kg, 21 mg/kg, 22 mg/kg, 23 mg/kg, 24 mg/kg, 25 mg/kg, 30 mg/kg, 35 mg/kg, 40 mg/kg, 45 mg/kg, 50 mg/kg, 55 mg/kg, 60 mg/kg, 65 mg/kg, 70 mg/kg, 75 mg/kg, 80 mg/kg, 85 mg/kg, 90 mg/kg, 95 mg/kg, 100 mg/kg, 105 mg/kg, 110 mg/kg, 115 mg/kg, 120 mg/kg, 125 mg/kg, 130 mg/kg, 135 mg/kg, 140 mg/kg, 145 mg/kg, 150 mg/kg, 155 mg/kg, 160 mg/kg, 165 mg/kg, 170 mg/kg, 175 mg/kg, 180 mg/kg, 185 mg/kg, 190 mg/kg, 195 mg/kg, 200 mg/kg, and any fraction or integer in between any two of the preceding amounts.

In certain embodiments, an nNOS inhibitor composition comprising one or more dsRNAs each comprising at least 30 base pairs, may be administered as a suitable dosage form, as described herein, to treat, ameliorate, and/or reduce the severity of sarcoma or one or more symptoms thereof in a sarcoma subject. In some embodiments, an nNOS inhibitor composition comprising one or more dsRNAs, each comprising at least 30 base pairs, may be administered to a sarcoma subject to treat, ameliorate, and/or reduce the severity of sarcoma or one or more symptoms thereof in the sarcoma subject: (i) via a route selected from the group consisting of buccally, inhalation, intraarterially, intraarticularly, intracardially, intracerebroventricularly, intradermally, intramedullary, intramuscularly, intraperitoneally, intrathecally, intravenously, intraventricularly, nasally, ophthalmically, orally, rectally, sublabially, sublingually, subcutaneously, topically, and transdermally; and/or (ii) with a frequency selected from the group consisting of four times daily, three times daily, twice daily, once daily, once every two days, once every three days, once every four days, once every five days, once every six days, once a week, once every eight days, once every nine days, once every ten days, once every two weeks, once every three weeks, and once a month; and/or (iii) at a dosage (based on subject weight) selected from the group consisting of 0.1 μg/kg, 0.5 μg/kg, 1 μg/kg, 1.5 μg/kg, 2.0 μg/kg, 2.5 μg/kg, 3.0 μg/kg, 3.5 μg/kg, 4.0 μg/kg, 4.5 μg/kg, 5.0 μg/kg, 10 μg/kg, 15 μg/kg, 20 μg/kg, 25 μg/kg, 30 μg/kg, 35 μg/kg, 40 μg/kg, 45 μg/kg, 50 μg/kg, 55 μg/kg, 60 μg/kg, 65 μg/kg, 70 μg/kg, 75 μg/kg, 80 μg/kg, 85 μg/kg, 90 μg/kg, 95 μg/kg, 100 μg/kg, 150 μg/kg, 200 μg/kg, 250 μg/kg, 300 μg/kg, 350 μg/kg, 400 μg/kg, 450 μg/kg, 500 μg/kg, 550 μg/kg, 600 μg/kg, 650 μg/kg, 700 μg/kg, 750 μg/kg, 800 μg/kg, 850 μg/kg, 900 μg/kg, 1 mg/kg, 1.5 mg/kg, 2.0 mg/kg, 2.5 mg/kg, 3 mg/kg, 3.5 mg/kg, 4.0 mg/kg, 4.5 mg/kg, 5 mg/kg, 5.5 mg/kg, 6 mg/kg, 6.5 mg/kg, 7 mg/kg, 7.5 mg/kg, 8 mg/kg, 8.5 mg/kg, 9 mg/kg, 9.5 mg/kg, 10 mg/kg 10.5 mg/kg, 11 mg/kg, 11.5 mg/kg, 12 mg/kg, 12.5 mg/kg, 13 mg/kg, 13.5 mg/kg, 14 mg/kg, 14.5 mg/kg, 15 mg/kg, 16 mg/kg, 17 mg/kg, 18 mg/kg, 19 mg/kg, 20 mg/kg, 21 mg/kg, 22 mg/kg, 23 mg/kg, 24 mg/kg, 25 mg/kg, 30 mg/kg, 35 mg/kg, 40 mg/kg, 45 mg/kg, 50 mg/kg, 55 mg/kg, 60 mg/kg, 65 mg/kg, 70 mg/kg, 75 mg/kg, 80 mg/kg, 85 mg/kg, 90 mg/kg, 95 mg/kg, 100 mg/kg, 105 mg/kg, 110 mg/kg, 115 mg/kg, 120 mg/kg, 125 mg/kg, 130 mg/kg, 135 mg/kg, 140 mg/kg, 145 mg/kg, 150 mg/kg, 155 mg/kg, 160 mg/kg, 165 mg/kg, 170 mg/kg, 175 mg/kg, 180 mg/kg, 185 mg/kg, 190 mg/kg, 195 mg/kg, 200 mg/kg, and any fraction or integer in between any two of the preceding amounts.

In some embodiments, an nNOS inhibitor composition comprising one or more siRNAs, may be administered as a suitable dosage form, as described herein, to treat, ameliorate, and/or reduce the severity of sarcoma or one or more symptoms thereof in a sarcoma subject. In certain embodiments, an nNOS inhibitor composition comprising one or more siRNAs selected from the group consisting of SEQ ID NO. 4, SEQ ID NO. 5, SEQ ID NO. 6, SEQ ID NO. 7, SEQ ID NO. 8, and any combination thereof, may be administered as a suitable dosage form, as described herein, to treat, ameliorate, and/or reduce the severity of sarcoma or one or more symptoms thereof in a sarcoma subject. In some embodiments, an nNOS inhibitor composition comprising one or more siRNAs selected from the group consisting of SEQ ID NO. 4, SEQ ID NO. 5, SEQ ID NO. 6, SEQ ID NO. 7, SEQ ID NO. 8, and any combination thereof, may be administered to a sarcoma subject to treat, ameliorate, and/or reduce the severity of sarcoma or one or more symptoms thereof in the sarcoma subject: (i) via a route selected from the group consisting of buccally, inhalation, intraarterially, intraarticularly, intracardially, intracerebroventricularly, intradermally, intramedullary, intramuscularly, intraperitoneally, intrathecally, intravenously, intraventricularly, nasally, ophthalmically, orally, rectally, sublabially, sublingually, subcutaneously, topically, and transdermally; and/or (ii) with a frequency selected from the group consisting of four times daily, three times daily, twice daily, once daily, once every two days, once every three days, once every four days, once every five days, once every six days, once a week, once every eight days, once every nine days, once every ten days, once every two weeks, once every three weeks, and once a month; and/or (iii) at a dosage (based on subject weight) selected from the group consisting of 0.1 μg/kg, 0.5 μg/kg, 1 μg/kg, 1.5 μg/kg, 2.0 μg/kg, 2.5 μg/kg, 3.0 μg/kg, 3.5 μg/kg, 4.0 μg/kg, 4.5 μg/kg, 5.0 μg/kg, 10 μg/kg, 15 μg/kg, 20 μg/kg, 25 μg/kg, 30 μg/kg, 35 μg/kg, 40 μg/kg, 45 μg/kg, 50 μg/kg, 55 μg/kg, 60 μg/kg, 65 μg/kg, 70 μg/kg, 75 μg/kg, 80 μg/kg, 85 μg/kg, 90 μg/kg, 95 μg/kg, 100 μg/kg, 150 μg/kg, 200 μg/kg, 250 μg/kg, 300 μg/kg, 350 μg/kg, 400 μg/kg, 450 μg/kg, 500 μg/kg, 550 μg/kg, 600 μg/kg, 650 μg/kg, 700 μg/kg, 750 μg/kg, 800 μg/kg, 850 μg/kg, 900 μg/kg, 1 mg/kg, 1.5 mg/kg, 2.0 mg/kg, 2.5 mg/kg, 3 mg/kg, 3.5 mg/kg, 4.0 mg/kg, 4.5 mg/kg, 5 mg/kg, 5.5 mg/kg, 6 mg/kg, 6.5 mg/kg, 7 mg/kg, 7.5 mg/kg, 8 mg/kg, 8.5 mg/kg, 9 mg/kg, 9.5 mg/kg, 10 mg/kg 10.5 mg/kg, 11 mg/kg, 11.5 mg/kg, 12 mg/kg, 12.5 mg/kg, 13 mg/kg, 13.5 mg/kg, 14 mg/kg, 14.5 mg/kg, 15 mg/kg, 16 mg/kg, 17 mg/kg, 18 mg/kg, 19 mg/kg, 20 mg/kg, 21 mg/kg, 22 mg/kg, 23 mg/kg, 24 mg/kg, 25 mg/kg, 30 mg/kg, 35 mg/kg, 40 mg/kg, 45 mg/kg, 50 mg/kg, 55 mg/kg, 60 mg/kg, 65 mg/kg, 70 mg/kg, 75 mg/kg, 80 mg/kg, 85 mg/kg, 90 mg/kg, 95 mg/kg, 100 mg/kg, 105 mg/kg, 110 mg/kg, 115 mg/kg, 120 mg/kg, 125 mg/kg, 130 mg/kg, 135 mg/kg, 140 mg/kg, 145 mg/kg, 150 mg/kg, 155 mg/kg, 160 mg/kg, 165 mg/kg, 170 mg/kg, 175 mg/kg, 180 mg/kg, 185 mg/kg, 190 mg/kg, 195 mg/kg, 200 mg/kg, and any fraction or integer in between any two of the preceding amounts.

In some embodiments, an nNOS inhibitor composition comprising one or more shRNAs, may be administered as a suitable dosage form, as described herein, to treat, ameliorate, and/or reduce the severity of sarcoma or one or more symptoms thereof in a sarcoma subject. In certain embodiments, an nNOS inhibitor composition comprising one or more shRNAs selected from the group consisting of SEQ ID NO. 9, SEQ ID NO. 10, SEQ ID NO. 11, SEQ ID NO. 12, SEQ ID NO. 13, SEQ ID NO. 14, and any combination thereof, may be administered as a suitable dosage form, as described herein, to treat, ameliorate, and/or reduce the severity of sarcoma or one or more symptoms thereof in a sarcoma subject. In some embodiments, an nNOS inhibitor composition comprising one or more shRNAs selected from the group consisting of SEQ ID NO. 9, SEQ ID NO. 10, SEQ ID NO. 11, SEQ ID NO. 12, SEQ ID NO. 13, SEQ ID NO. 14, and any combination thereof, may be administered to a sarcoma subject to treat, ameliorate, and/or reduce the severity of sarcoma or one or more symptoms thereof in the sarcoma subject: (i) via a route selected from the group consisting of buccally, inhalation, intraarterially, intraarticularly, intracardially, intracerebroventricularly, intradermally, intramedullary, intramuscularly, intraperitoneally, intrathecally, intravenously, intraventricularly, nasally, ophthalmically, orally, rectally, sublabially, sublingually, subcutaneously, topically, and transdermally; and/or (ii) with a frequency selected from the group consisting of four times daily, three times daily, twice daily, once daily, once every two days, once every three days, once every four days, once every five days, once every six days, once a week, once every eight days, once every nine days, once every ten days, once every two weeks, once every three weeks, and once a month; and/or (iii) at a dosage (based on subject weight) selected from the group consisting of 0.1 μg/kg, 0.5 μg/kg, 1 μg/kg, 1.5 μg/kg, 2.0 μg/kg, 2.5 μg/kg, 3.0 μg/kg, 3.5 μg/kg, 4.0 μg/kg, 4.5 μg/kg, 5.0 μg/kg, 10 μg/kg, 15 μg/kg, 20 μg/kg, 25 μg/kg, 30 μg/kg, 35 μg/kg, 40 μg/kg, 45 μg/kg, 50 μg/kg, 55 μg/kg, 60 μg/kg, 65 μg/kg, 70 μg/kg, 75 μg/kg, 80 μg/kg, 85 μg/kg, 90 μg/kg, 95 μg/kg, 100 μg/kg, 150 μg/kg, 200 μg/kg, 250 μg/kg, 300 μg/kg, 350 μg/kg, 400 μg/kg, 450 μg/kg, 500 μg/kg, 550 μg/kg, 600 μg/kg, 650 μg/kg, 700 μg/kg, 750 μg/kg, 800 μg/kg, 850 μg/kg, 900 μg/kg, 1 mg/kg, 1.5 mg/kg, 2.0 mg/kg, 2.5 mg/kg, 3 mg/kg, 3.5 mg/kg, 4.0 mg/kg, 4.5 mg/kg, 5 mg/kg, 5.5 mg/kg, 6 mg/kg, 6.5 mg/kg, 7 mg/kg, 7.5 mg/kg, 8 mg/kg, 8.5 mg/kg, 9 mg/kg, 9.5 mg/kg, 10 mg/kg 10.5 mg/kg, 11 mg/kg, 11.5 mg/kg, 12 mg/kg, 12.5 mg/kg, 13 mg/kg, 13.5 mg/kg, 14 mg/kg, 14.5 mg/kg, 15 mg/kg, 16 mg/kg, 17 mg/kg, 18 mg/kg, 19 mg/kg, 20 mg/kg, 21 mg/kg, 22 mg/kg, 23 mg/kg, 24 mg/kg, 25 mg/kg, 30 mg/kg, 35 mg/kg, 40 mg/kg, 45 mg/kg, 50 mg/kg, 55 mg/kg, 60 mg/kg, 65 mg/kg, 70 mg/kg, 75 mg/kg, 80 mg/kg, 85 mg/kg, 90 mg/kg, 95 mg/kg, 100 mg/kg, 105 mg/kg, 110 mg/kg, 115 mg/kg, 120 mg/kg, 125 mg/kg, 130 mg/kg, 135 mg/kg, 140 mg/kg, 145 mg/kg, 150 mg/kg, 155 mg/kg, 160 mg/kg, 165 mg/kg, 170 mg/kg, 175 mg/kg, 180 mg/kg, 185 mg/kg, 190 mg/kg, 195 mg/kg, 200 mg/kg, and any fraction or integer in between any two of the preceding amounts.

In some embodiments, an nNOS inhibitor composition comprising one or more miRNAs, may be administered as a suitable dosage form, as described herein, to treat, ameliorate, and/or reduce the severity of sarcoma or one or more symptoms thereof in a sarcoma subject. In certain embodiments, an nNOS inhibitor composition comprising one or more miRNAs selected from the group consisting of miR-146a, hsa-miR-146a-5p, hsa-miR-1273f, hsa-miR-143-3p, hsa-miR-147a, hsa-miR-3622a-5p, hsa-miR-3911, hsa-miR-4269, hsa-miR-4708-5p, hsa-miR-4710, hsa-miR-4770, hsa-miR-4792, hsa-miR-574-5p, hsa-miR-6088, hsa-miR-644a, hsa-miR-6715b-5p, hsa-miR-6768-5p, hsa-miR-6867-5p, hsa-miR-7855-5p, hsa-miR-8485, and any combination thereof, may be administered as a suitable dosage form, as described herein, to treat, ameliorate, and/or reduce the severity of sarcoma or one or more symptoms thereof in a sarcoma subject. In some embodiments, an nNOS inhibitor composition comprising one or more miRNAs selected from the group consisting of miR-146a, hsa-miR-146a-5p, hsa-miR-1273f, hsa-miR-143-3p, hsa-miR-147a, hsa-miR-3622a-5p, hsa-miR-3911, hsa-miR-4269, hsa-miR-4708-5p, hsa-miR-4710, hsa-miR-4770, hsa-miR-4792, hsa-miR-574-5p, hsa-miR-6088, hsa-miR-644a, hsa-miR-6715b-5p, hsa-miR-6768-5p, hsa-miR-6867-5p, hsa-miR-7855-5p, hsa-miR-8485, and any combination thereof, may be administered to a sarcoma subject to treat, ameliorate, and/or reduce the severity of sarcoma or one or more symptoms thereof in the sarcoma subject: (i) via a route selected from the group consisting of buccally, inhalation, intraarterially, intraarticularly, intracardially, intracerebroventricularly, intradermally, intramedullary, intramuscularly, intraperitoneally, intrathecally, intravenously, intraventricularly, nasally, ophthalmically, orally, rectally, sublabially, sublingually, subcutaneously, topically, and transdermally; and/or (ii) with a frequency selected from the group consisting of four times daily, three times daily, twice daily, once daily, once every two days, once every three days, once every four days, once every five days, once every six days, once a week, once every eight days, once every nine days, once every ten days, once every two weeks, once every three weeks, and once a month; and/or (iii) at a dosage (based on subject weight) selected from the group consisting of 0.1 μg/kg, 0.5 μg/kg, 1 μg/kg, 1.5 μg/kg, 2.0 μg/kg, 2.5 μg/kg, 3.0 μg/kg, 3.5 μg/kg, 4.0 μg/kg, 4.5 μg/kg, 5.0 μg/kg, 10 μg/kg, 15 μg/kg, 20 μg/kg, 25 μg/kg, 30 μg/kg, 35 μg/kg, 40 μg/kg, 45 μg/kg, 50 μg/kg, 55 μg/kg, 60 μg/kg, 65 μg/kg, 70 μg/kg, 75 μg/kg, 80 μg/kg, 85 μg/kg, 90 μg/kg, 95 μg/kg, 100 μg/kg, 150 μg/kg, 200 μg/kg, 250 μg/kg, 300 μg/kg, 350 μg/kg, 400 μg/kg, 450 μg/kg, 500 μg/kg, 550 μg/kg, 600 μg/kg, 650 μg/kg, 700 μg/kg, 750 μg/kg, 800 μg/kg, 850 μg/kg, 900 μg/kg, 1 mg/kg, 1.5 mg/kg, 2.0 mg/kg, 2.5 mg/kg, 3 mg/kg, 3.5 mg/kg, 4.0 mg/kg, 4.5 mg/kg, 5 mg/kg, 5.5 mg/kg, 6 mg/kg, 6.5 mg/kg, 7 mg/kg, 7.5 mg/kg, 8 mg/kg, 8.5 mg/kg, 9 mg/kg, 9.5 mg/kg, 10 mg/kg 10.5 mg/kg, 11 mg/kg, 11.5 mg/kg, 12 mg/kg, 12.5 mg/kg, 13 mg/kg, 13.5 mg/kg, 14 mg/kg, 14.5 mg/kg, 15 mg/kg, 16 mg/kg, 17 mg/kg, 18 mg/kg, 19 mg/kg, 20 mg/kg, 21 mg/kg, 22 mg/kg, 23 mg/kg, 24 mg/kg, 25 mg/kg, 30 mg/kg, 35 mg/kg, 40 mg/kg, 45 mg/kg, 50 mg/kg, 55 mg/kg, 60 mg/kg, 65 mg/kg, 70 mg/kg, 75 mg/kg, 80 mg/kg, 85 mg/kg, 90 mg/kg, 95 mg/kg, 100 mg/kg, 105 mg/kg, 110 mg/kg, 115 mg/kg, 120 mg/kg, 125 mg/kg, 130 mg/kg, 135 mg/kg, 140 mg/kg, 145 mg/kg, 150 mg/kg, 155 mg/kg, 160 mg/kg, 165 mg/kg, 170 mg/kg, 175 mg/kg, 180 mg/kg, 185 mg/kg, 190 mg/kg, 195 mg/kg, 200 mg/kg, and any fraction or integer in between any two of the preceding amounts.

In some embodiments, an nNOS inhibitor composition comprising one or more CRISPR/Cas systems, may be administered as a suitable dosage form, as described herein, to treat, ameliorate, and/or reduce the severity of sarcoma or one or more symptoms thereof in a sarcoma subject. In certain embodiments, an nNOS inhibitor composition comprising one or more CRISPR/Cas systems comprising an endonuclease selected from the group consisting of Cas1, Cas2, Cas3, Cas4, Cas5, Cas6, Cas7, Cas8a, Cas8b, Cas8c, Cas9, Cas10, Cas10d, Cas12, Cas12a, Cas12b, Cas12c, Cas12d, Cas12e, Cas12f, Cas12g, Cas12h, Cas12i, Cas12k, C2c4, C2c8, C2c9, Cas13, Cas13a, Cas13b, Cas13c, Cas13d, Cas13x.1, Cse1, Cse2, Csy1, Csy2, Csy3, GSU0054, Csm2, Cmr5, Csx10, Csx11, Csf1, and Csn2, may be administered as a suitable dosage form, as described herein, to treat, ameliorate, and/or reduce the severity of sarcoma or one or more symptoms thereof in a sarcoma subject. In some embodiments, an nNOS inhibitor composition comprising one or more CRISPR/Cas systems comprising an endonuclease selected from the group consisting of Cas1, Cas2, Cas3, Cas4, Cas5, Cas6, Cas7, Cas8a, Cas8b, Cas8c, Cas9, Cas10, Cas10d, Cas12, Cas12a, Cas12b, Cas12c, Cas12d, Cas12e, Cas12f, Cas12g, Cas12h, Cas12i, Cas12k, C2c4, C2c8, C2c9, Cas13, Cas13a, Cas13b, Cas13c, Cas13d, Cas13x.1, Cse1, Cse2, Csy1, Csy2, Csy3, GSU0054, Csm2, Cmr5, Csx10, Csx11, Csf1, and Csn2, may be administered to a sarcoma subject to treat, ameliorate, and/or reduce the severity of sarcoma or one or more symptoms thereof in the sarcoma subject: (i) via a route selected from the group consisting of buccally, inhalation, intraarterially, intraarticularly, intracardially, intracerebroventricularly, intradermally, intramedullary, intramuscularly, intraperitoneally, intrathecally, intravenously, intraventricularly, nasally, ophthalmically, orally, rectally, sublabially, sublingually, subcutaneously, topically, and transdermally; and/or (ii) with a frequency selected from the group consisting of four times daily, three times daily, twice daily, once daily, once every two days, once every three days, once every four days, once every five days, once every six days, once a week, once every eight days, once every nine days, once every ten days, once every two weeks, once every three weeks, and once a month; and/or (iii) at a dosage (based on subject weight) selected from the group consisting of 0.1 μg/kg, 0.5 μg/kg, 1 μg/kg, 1.5 μg/kg, 2.0 μg/kg, 2.5 μg/kg, 3.0 μg/kg, 3.5 μg/kg, 4.0 μg/kg, 4.5 μg/kg, 5.0 μg/kg, 10 μg/kg, 15 μg/kg, 20 μg/kg, 25 μg/kg, 30 μg/kg, 35 μg/kg, 40 μg/kg, 45 μg/kg, 50 μg/kg, 55 μg/kg, 60 μg/kg, 65 μg/kg, 70 μg/kg, 75 μg/kg, 80 μg/kg, 85 μg/kg, 90 μg/kg, 95 μg/kg, 100 μg/kg, 150 μg/kg, 200 μg/kg, 250 μg/kg, 300 μg/kg, 350 μg/kg, 400 μg/kg, 450 μg/kg, 500 μg/kg, 550 μg/kg, 600 μg/kg, 650 μg/kg, 700 μg/kg, 750 μg/kg, 800 μg/kg, 850 μg/kg, 900 μg/kg, 1 mg/kg, 1.5 mg/kg, 2.0 mg/kg, 2.5 mg/kg, 3 mg/kg, 3.5 mg/kg, 4.0 mg/kg, 4.5 mg/kg, 5 mg/kg, 5.5 mg/kg, 6 mg/kg, 6.5 mg/kg, 7 mg/kg, 7.5 mg/kg, 8 mg/kg, 8.5 mg/kg, 9 mg/kg, 9.5 mg/kg, 10 mg/kg 10.5 mg/kg, 11 mg/kg, 11.5 mg/kg, 12 mg/kg, 12.5 mg/kg, 13 mg/kg, 13.5 mg/kg, 14 mg/kg, 14.5 mg/kg, 15 mg/kg, 16 mg/kg, 17 mg/kg, 18 mg/kg, 19 mg/kg, 20 mg/kg, 21 mg/kg, 22 mg/kg, 23 mg/kg, 24 mg/kg, 25 mg/kg, 30 mg/kg, 35 mg/kg, 40 mg/kg, 45 mg/kg, 50 mg/kg, 55 mg/kg, 60 mg/kg, 65 mg/kg, 70 mg/kg, 75 mg/kg, 80 mg/kg, 85 mg/kg, 90 mg/kg, 95 mg/kg, 100 mg/kg, 105 mg/kg, 110 mg/kg, 115 mg/kg, 120 mg/kg, 125 mg/kg, 130 mg/kg, 135 mg/kg, 140 mg/kg, 145 mg/kg, 150 mg/kg, 155 mg/kg, 160 mg/kg, 165 mg/kg, 170 mg/kg, 175 mg/kg, 180 mg/kg, 185 mg/kg, 190 mg/kg, 195 mg/kg, 200 mg/kg, and any fraction or integer in between any two of the preceding amounts.

In some embodiments, an nNOS inhibitor composition comprising one or more antibodies, may be administered as a suitable dosage form, as described herein, to treat, ameliorate, and/or reduce the severity of sarcoma or one or more symptoms thereof in a sarcoma subject. In certain embodiments, an nNOS inhibitor composition comprising one or more antibodies selected from the group consisting of 37-2800 (from ThermoFisher), EP1855Y (from abeam), EPR 24351-6 (from abeam), and any combination thereof, may be administered as a suitable dosage form, as described herein, to treat, ameliorate, and/or reduce the severity of sarcoma or one or more symptoms thereof in a sarcoma subject. In some embodiments, an nNOS inhibitor composition comprising one or more antibodies selected from the group consisting of 37-2800 (from ThermoFisher), EP1855Y (from abeam), EPR 24351-6 (from abeam), and any combination thereof, may be administered to a sarcoma subject to treat, ameliorate, and/or reduce the severity of sarcoma or one or more symptoms thereof in the sarcoma subject: (i) via a route selected from the group consisting of buccally, inhalation, intraarterially, intraarticularly, intracardially, intracerebroventricularly, intradermally, intramedullary, intramuscularly, intraperitoneally, intrathecally, intravenously, intraventricularly, nasally, ophthalmically, orally, rectally, sublabially, sublingually, subcutaneously, topically, and transdermally; and/or (ii) with a frequency selected from the group consisting of four times daily, three times daily, twice daily, once daily, once every two days, once every three days, once every four days, once every five days, once every six days, once a week, once every eight days, once every nine days, once every ten days, once every two weeks, once every three weeks, and once a month; and/or (iii) at a dosage (based on subject weight) selected from the group consisting of 0.1 μg/kg, 0.5 μg/kg, 1 μg/kg, 1.5 μg/kg, 2.0 μg/kg, 2.5 μg/kg, 3.0 μg/kg, 3.5 μg/kg, 4.0 μg/kg, 4.5 μg/kg, 5.0 μg/kg, 10 μg/kg, 15 μg/kg, 20 μg/kg, 25 μg/kg, 30 μg/kg, 35 μg/kg, 40 μg/kg, 45 μg/kg, 50 μg/kg, 55 μg/kg, 60 μg/kg, 65 μg/kg, 70 μg/kg, 75 μg/kg, 80 μg/kg, 85 μg/kg, 90 μg/kg, 95 μg/kg, 100 μg/kg, 150 μg/kg, 200 μg/kg, 250 μg/kg, 300 μg/kg, 350 μg/kg, 400 μg/kg, 450 μg/kg, 500 μg/kg, 550 μg/kg, 600 μg/kg, 650 μg/kg, 700 μg/kg, 750 μg/kg, 800 μg/kg, 850 μg/kg, 900 μg/kg, 1 mg/kg, 1.5 mg/kg, 2.0 mg/kg, 2.5 mg/kg, 3 mg/kg, 3.5 mg/kg, 4.0 mg/kg, 4.5 mg/kg, 5 mg/kg, 5.5 mg/kg, 6 mg/kg, 6.5 mg/kg, 7 mg/kg, 7.5 mg/kg, 8 mg/kg, 8.5 mg/kg, 9 mg/kg, 9.5 mg/kg, 10 mg/kg 10.5 mg/kg, 11 mg/kg, 11.5 mg/kg, 12 mg/kg, 12.5 mg/kg, 13 mg/kg, 13.5 mg/kg, 14 mg/kg, 14.5 mg/kg, 15 mg/kg, 16 mg/kg, 17 mg/kg, 18 mg/kg, 19 mg/kg, 20 mg/kg, 21 mg/kg, 22 mg/kg, 23 mg/kg, 24 mg/kg, 25 mg/kg, 30 mg/kg, 35 mg/kg, 40 mg/kg, 45 mg/kg, 50 mg/kg, 55 mg/kg, 60 mg/kg, 65 mg/kg, 70 mg/kg, 75 mg/kg, 80 mg/kg, 85 mg/kg, 90 mg/kg, 95 mg/kg, 100 mg/kg, 105 mg/kg, 110 mg/kg, 115 mg/kg, 120 mg/kg, 125 mg/kg, 130 mg/kg, 135 mg/kg, 140 mg/kg, 145 mg/kg, 150 mg/kg, 155 mg/kg, 160 mg/kg, 165 mg/kg, 170 mg/kg, 175 mg/kg, 180 mg/kg, 185 mg/kg, 190 mg/kg, 195 mg/kg, 200 mg/kg, and any fraction or integer in between any two of the preceding amounts.

In some embodiments, an nNOS inhibitor composition comprising one or more amino acid-based compounds, may be administered as a suitable dosage form, as described herein, to treat, ameliorate, and/or reduce the severity of sarcoma or one or more symptoms thereof in a sarcoma subject. In certain embodiments, an nNOS inhibitor composition comprising one or more amino acid-compounds that bind an active site of nNOS selected from the group consisting of Phenylalanine 589, Tryptophan 592, Tyrosine 593, Glutamate 597, Aspartate 602, Arginine 601, Methionine 341, Histidine 342, and Tyrosine711, may be administered as a suitable dosage form, as described herein, to treat, ameliorate, and/or reduce the severity of sarcoma or one or more symptoms thereof in a sarcoma subject. In some embodiments, an nNOS inhibitor composition comprising one or more amino acid-compounds that bind an active site of nNOS selected from the group consisting of Phenylalanine 589, Tryptophan 592, Tyrosine 593, Glutamate 597, Aspartate 602, Arginine 601, Methionine 341, Histidine 342, and Tyrosine711, may be administered to a sarcoma subject to treat, ameliorate, and/or reduce the severity of sarcoma or one or more symptoms thereof in the sarcoma subject: (i) via a route selected from the group consisting of buccally, inhalation, intraarterially, intraarticularly, intracardially, intracerebroventricularly, intradermally, intramedullary, intramuscularly, intraperitoneally, intrathecally, intravenously, intraventricularly, nasally, ophthalmically, orally, rectally, sublabially, sublingually, subcutaneously, topically, and transdermally; and/or (ii) with a frequency selected from the group consisting of four times daily, three times daily, twice daily, once daily, once every two days, once every three days, once every four days, once every five days, once every six days, once a week, once every eight days, once every nine days, once every ten days, once every two weeks, once every three weeks, and once a month; and/or (iii) at a dosage (based on subject weight) selected from the group consisting of 0.1 μg/kg, 0.5 μg/kg, 1 μg/kg, 1.5 μg/kg, 2.0 μg/kg, 2.5 μg/kg, 3.0 μg/kg, 3.5 μg/kg, 4.0 μg/kg, 4.5 μg/kg, 5.0 μg/kg, 10 μg/kg, 15 μg/kg, 20 μg/kg, 25 μg/kg, 30 μg/kg, 35 μg/kg, 40 μg/kg, 45 μg/kg, 50 μg/kg, 55 μg/kg, 60 μg/kg, 65 μg/kg, 70 μg/kg, 75 μg/kg, 80 μg/kg, 85 μg/kg, 90 μg/kg, 95 μg/kg, 100 μg/kg, 150 μg/kg, 200 μg/kg, 250 μg/kg, 300 μg/kg, 350 μg/kg, 400 μg/kg, 450 μg/kg, 500 μg/kg, 550 μg/kg, 600 μg/kg, 650 μg/kg, 700 μg/kg, 750 μg/kg, 800 μg/kg, 850 μg/kg, 900 μg/kg, 1 mg/kg, 1.5 mg/kg, 2.0 mg/kg, 2.5 mg/kg, 3 mg/kg, 3.5 mg/kg, 4.0 mg/kg, 4.5 mg/kg, 5 mg/kg, 5.5 mg/kg, 6 mg/kg, 6.5 mg/kg, 7 mg/kg, 7.5 mg/kg, 8 mg/kg, 8.5 mg/kg, 9 mg/kg, 9.5 mg/kg, 10 mg/kg 10.5 mg/kg, 11 mg/kg, 11.5 mg/kg, 12 mg/kg, 12.5 mg/kg, 13 mg/kg, 13.5 mg/kg, 14 mg/kg, 14.5 mg/kg, 15 mg/kg, 16 mg/kg, 17 mg/kg, 18 mg/kg, 19 mg/kg, 20 mg/kg, 21 mg/kg, 22 mg/kg, 23 mg/kg, 24 mg/kg, 25 mg/kg, 30 mg/kg, 35 mg/kg, 40 mg/kg, 45 mg/kg, 50 mg/kg, 55 mg/kg, 60 mg/kg, 65 mg/kg, 70 mg/kg, 75 mg/kg, 80 mg/kg, 85 mg/kg, 90 mg/kg, 95 mg/kg, 100 mg/kg, 105 mg/kg, 110 mg/kg, 115 mg/kg, 120 mg/kg, 125 mg/kg, 130 mg/kg, 135 mg/kg, 140 mg/kg, 145 mg/kg, 150 mg/kg, 155 mg/kg, 160 mg/kg, 165 mg/kg, 170 mg/kg, 175 mg/kg, 180 mg/kg, 185 mg/kg, 190 mg/kg, 195 mg/kg, 200 mg/kg, and any fraction or integer in between any two of the preceding amounts.

In some embodiments, a sarcoma subject, as described herein, may be a female child or a male child. In certain embodiments, a sarcoma subject, as described herein, may be a female adult or a male adult.

In certain embodiments, a sarcoma subject, as described herein, may have a sarcoma selected from the group consisting of an alveolar soft-part sarcoma, an angiofibroma, an angiolipoma, an angiomyelolipoma, an angiomyolipoma, an angiomyxoma, an angiosarcoma, a chondrosarcoma, a clear cell sarcoma, a dermatofibrosarcoma protuberans, a desmoid tumor, a desmoplastic small round cell tumor, an elastoma, an epithelioid sarcoma, a Ewing's sarcoma, an extraskeletal myxoid chondrosarcoma, a fibrosarcoma, a gastrointestinal stromal tumor, a giant cell tumor of a tendon sheath, a glomus tissue tumor, a granular cell tumor, a hemangioma, a hemangiopericytoma, a hibernoma, a Kaposi's sarcoma, a leiomyoma, a leiomyosarcoma, a lipoblastoma, a lipoblastomatosis, a lipoma, a lipomatosis, a liposarcoma, a lymphangioleiomyomatosis, a malignant fibrous histiocytoma, a malignant peripheral nerve sheath tumor, a myeloid sarcoma, a myoepithelial tumor of soft tissue, a myxofibrosarcoma, a myxoma, a neurofibroma, a neurofibrosarcoma, an ossifying fibromyxoid tumor, an osteosarcoma, a perivascular epithelioid cell tumor, a pigmented villonodular synovitis, a pleomorphic sarcoma, a rhabdomyosarcoma, a schwannoma, a soft tissue sarcoma, a solitary fibrous tumor, a spindle cell sarcoma, a sugar cell tumor, a synovial cell sarcoma, an undifferentiated pleomorphic sarcoma, and a uterine sarcoma.

In some embodiments, a sarcoma subject, as described herein, may have an abnormal expression of one or more antigens compared to an equivalent subject that does not have a sarcoma. As used herein, the term “equivalent subject,” refers to a subject of the same sex (male/female) and age range (child/adult) of the sarcoma subject. For purposes of disclosure, the term “antigen,” when used with respect to abnormal expression, may define one or more CD antigens, such as, for example, CD15, CD44, CD70, CD133, as well as any CD antigen that has an abnormal expression in a sarcoma disclosed herein that would be readily envisaged by one skilled in the art. In certain embodiments, abnormal expression of an antigen may define an abnormal expression of CD15, an abnormal expression of CD44, an abnormal expression of CD70, an abnormal expression of CD133, an abnormal expression of a CD antigen that has an abnormal expression in a sarcoma disclosed herein that would be readily envisaged by one skilled in the art, and any combination thereof. In some embodiments, an “abnormal expression,” can refer to a reduced expression of an antigen, as described herein. In certain embodiments, an “abnormal expression,” can refer to an overexpression of an antigen, as described herein. Whether “abnormal expression,” refers to a reduced expression of an antigen or an overexpression of an antigen would be immediately envisaged by the skilled artisan when viewing the term in the context of the particular antigen disclosed, as well as the sarcoma to which the abnormal expression of antigen refers to. For example, in the context of CD70 and Kaposi's sarcoma, one skilled in the art would immediately envisage that “abnormal expression” refers to a reduced expression of CD70. Treatment, amelioration, and/or reduction in the severity of a sarcoma or one or more symptoms thereof may be characterized by a restoration to a normal level of expression of one or more antigens from their abnormal state, as described herein. For example, in the context of CD70 and Kaposi's sarcoma, treatment, amelioration, and/or reduction in the severity of a sarcoma or one or more symptoms thereof may be characterized by an increased expression of CD70.

In certain embodiments, a sarcoma subject, as described herein, may have an abnormal expression of one or more proteins compared to an equivalent subject that does not have a sarcoma. For purposes of disclosure, the term “protein,” when used with respect to abnormal expression, may define one or more proteins and/or enzymes, such as, for example, ALDH1A3, mTOR, p-mTOR, TSC-2, AKT, pAKT, RPS6, p-RPS6, Syp, VEGF, PIGF, as well as any protein or enzyme that has an abnormal expression in a sarcoma disclosed herein that would be readily envisaged by one skilled in the art. In certain embodiments, abnormal expression of a protein may define an abnormal expression of ALDH1A3, an abnormal expression of mTOR, an abnormal expression of p-mTOR, an abnormal expression of TSC-2, an abnormal expression of AKT, an abnormal expression of pAKT, an abnormal expression of RPS6, an abnormal expression of p-RPS6, an abnormal expression of Syp, an abnormal expression of VEGF, an abnormal expression of PIGF, an abnormal expression of a protein or enzyme that has an abnormal expression in a sarcoma disclosed herein that would be readily envisaged by one skilled in the art, and any combination thereof. In some embodiments, an “abnormal expression,” can refer to a reduced expression of a protein, as described herein. In certain embodiments, an “abnormal expression,” can refer to an overexpression of a protein, as described herein. Whether “abnormal expression,” refers to a reduced expression of a protein or an overexpression of a protein would be immediately envisaged by the skilled artisan when viewing the term in the context of the particular protein disclosed, as well as the sarcoma to which the abnormal expression of protein refers to. For example, in the context of VEGF and osteosarcoma, one skilled in the art would immediately envisage that “abnormal expression” refers to an overexpression of VEGF. Treatment, amelioration, and/or reduction in the severity of a sarcoma or one or more symptoms thereof may be characterized by a restoration to a normal level of expression of one or more proteins from their abnormal state, as described herein. For example, in the context of VEGF and osteosarcoma, treatment, amelioration, and/or reduction in the severity of a sarcoma or one or more symptoms thereof may be characterized by a reduced expression of VEGF.

In certain embodiments, a sarcoma subject, as described herein, may have an increased expression of one or more NOS isoforms compared to an equivalent subject that does not have a sarcoma. For purposes of disclosure, the term “NOS isoform,” refers to nNOS, iNOS, and/or eNOS. In certain embodiments, a sarcoma subject, as described herein, may have an increased expression of nNOS compared to an equivalent subject that does not have a sarcoma. In some embodiments, a sarcoma subject, as described herein, may have an increased expression of iNOS compared to an equivalent subject that does not have a sarcoma. In certain embodiments, a sarcoma subject, as described herein, may have an increased expression of eNOS compared to an equivalent subject that does not have a sarcoma. In some embodiments, a sarcoma subject, as described herein, may have an increased expression of nNOS and iNOS compared to an equivalent subject that does not have a sarcoma. In certain embodiments, a sarcoma subject, as described herein, may have an increased expression of nNOS and eNOS compared to an equivalent subject that does not have a sarcoma. In some embodiments, a sarcoma subject, as described herein, may have an increased expression of iNOS and eNOS compared to an equivalent subject that does not have a sarcoma. In certain embodiments, a sarcoma subject, as described herein, may have an increased expression of nNOS, iNOS, and eNOS compared to an equivalent subject that does not have a sarcoma. Whether a “NOS isoform,” has an increased expression would be immediately envisaged by the skilled artisan when viewing the term in the context of the particular NOS isoform disclosed, as well as the sarcoma to which the increased expression of NOS isoform refers to. For example, in the context of rhabdomyosarcoma, one skilled in the art would immediately envisage that iNOS has an increased expression. Treatment, amelioration, and/or reduction in the severity of a sarcoma or one or more symptoms thereof may be characterized by a reduced expression of one or more NOS isoforms from their increased state, as described herein. For example, in the context of rhabdomyosarcoma, treatment, amelioration, and/or reduction in the severity of a sarcoma or one or more symptoms thereof may be characterized by a reduced expression of iNOS. For purposes of disclosure, reduction/inhibition of the expression of one or more NOS isoforms may be characterized by a reduction in expression of one or more NOS isoforms by about at least 10%, about at least 20%, about at least 30%, or about at least 40%. In some embodiments, reduction/inhibition of the expression of one or more NOS isoforms may further be characterized by a lack reduction of other, non-implicated NOS isoforms, such as, for example, no more than about 10%, no more than about 5%, no more than about 2%, or no more than about 1% reduction/inhibition of the expression of non-implicated NOS isoforms. As used herein, the term “non-implicated NOS isoforms,” refers to NOS isoforms that do not have an increased expression in a particular sarcoma. By way of example, in the context of rhabdomyosarcoma, if iNOS has an increased expression, nNOS and eNOS would define “non-implicated”NOS isoforms. Whether a NOS isoform is a “non-implicated NOS isoform,” would be immediately envisaged by the skilled artisan when viewing the term in the context of the particular NOS isoform disclosed, as well as the sarcoma.

In some embodiments, a sarcoma subject, as described herein, may have an elevated level of one or more NO-indicators compared to an equivalent subject that does not have a sarcoma. As used herein, the term “NO-indicator,” can refer to 3-nitrotyrosine (3-Ntyr), NADPH-diaphorase, total nitrite concentration, total nitrate concentration, total nitrite and nitrate concentration, as well as any NO-indicator that has an elevated level in a sarcoma disclosed herein that would be readily envisaged by one skilled in the art. For example, in the context of Kaposi's sarcoma, one skilled in the art would immediately envisage that 3-Ntyr can be present at an elevated level. Treatment, amelioration, and/or reduction in the severity of a sarcoma or one or more symptoms thereof may be characterized by a reduction in the level of one or more NO-indicators. For example, in the context of Kaposi's sarcoma, treatment, amelioration, and/or reduction in the severity of a sarcoma or one or more symptoms thereof may be characterized by a reduction in the level of 3-Ntyr.

In certain embodiments, a NOS inhibitor composition, as described herein, may be administered as a suitable dosage form, as described herein, to treat, ameliorate, and/or reduce the severity of a sarcoma or one or more symptoms thereof, as an alternative to a conventional therapy for treating, ameliorating, and/or reducing the severity of a sarcoma or one or more symptoms thereof. For purposes of disclosure, a “conventional therapy,” as described herein, may refer to a treatment that would be recommended by one skilled in the art to treat a sarcoma, and may define one or more of radiation, chemotherapy, immunotherapy, cell-based therapy, gene therapy, and the like—one skilled in the art would immediately envisage a “conventional therapy” based on the particular sarcoma disclosed, as well as characteristics of a sarcoma subject, such as, for example, the subject's sex, age, stage of the sarcoma, genotype of the sarcoma, and the like. Treatment, amelioration, and/or reduction in the severity of a sarcoma or one or more symptoms thereof may be characterized by an improved outcome compared to a conventional therapy, as described herein. For purposes of the disclosure, an “improved outcome” may define any one of the following: (i) a larger reduction in tumor volume and/or mass compared to a subject administered an equivalent dose of a conventional therapy; (ii) an improvement in the expected rate of survival of a subject, at, for example, 1 month, 3 months, 6 months, 9 months, 1 year, 2 years, 3 years, 5 years, 10 years, or more, compared to the expected rate of survival of a subject administered an equivalent dose of a conventional therapy; (iii) a reduction in the prevalence of side effects resulting from the NOS inhibitor composition compared to a subject administered an equivalent dose of a conventional therapy; (iv) a reduction in the severity of side effects resulting from the NOS inhibitor composition compared to a subject administered an equivalent dose of a conventional therapy; (v) a reduction in the dosage required to achieve an equivalent reduction in tumor volume compared to a subject administered a conventional therapy; (vi) a reduction in the dosage required to achieve an equivalent expected rate of survival of a subject compared to a subject administered a conventional therapy; (vii) a reduction in the likelihood of a recurrence of the sarcoma occurring in a subject compared to a subject administered an equivalent dose of a conventional therapy; (viii) a reduction in the likelihood of a metastasis occurring in a subject compared to a subject administered an equivalent dose of a conventional therapy; (ix) a reduction in the time required to be at a medical facility for treatment compared to a subject administered an equivalent dose of a conventional therapy; (x) a reduction in the cost associated with treatment compared to a subject administered an equivalent dose of a conventional therapy; as well as any other improved outcome not expressly recited that would be readily envisaged by one skilled in the art.

In some embodiments, a NOS inhibitor composition, as described herein, may be administered as a suitable dosage form, as described herein, to treat, ameliorate, and/or reduce the severity of a sarcoma or one or more symptoms thereof, wherein the NOS inhibitor composition comprises a first chemical NOS inhibitor and a second chemical NOS inhibitor. In certain embodiments, the first chemical NOS inhibitor may be administered concurrently with the second chemical NOS inhibitor. In some embodiments, the first chemical NOS inhibitor may be administered prior to the second chemical NOS inhibitor. In certain embodiments, the first chemical NOS inhibitor may be administered subsequently to the second chemical NOS inhibitor. Treatment, amelioration, and/or reduction in the severity of a sarcoma or one or more symptoms thereof may be characterized by an improved outcome compared an alternative treatment. As used herein, the term “alternative treatment,” may define one or more of a conventional therapy, as described herein, a NOS inhibitor composition comprising a single chemical NOS inhibitor, as described herein, and a NOS inhibitor composition comprising a single biological NOS inhibitor, as described herein. For purposes of the disclosure, an “improved outcome” may define any one of the following: (i) a larger reduction in tumor volume and/or mass compared an alternative treatment; (ii) an improvement in the expected rate of survival of a subject, at, for example, 1 month, 3 months, 6 months, 9 months, 1 year, 2 years, 3 years, 5 years, 10 years, or more, compared to the expected rate of survival of a subject administered an alternative treatment; (iii) a reduction in the prevalence of side effects resulting from the NOS inhibitor composition comprising at least two chemical NOS inhibitors compared to a subject administered an alternative treatment; (iv) a reduction in the severity of side effects resulting from the NOS inhibitor composition comprising at least two chemical NOS inhibitors compared to a subject administered an alternative treatment; (v) a reduction in the dosage required to achieve an equivalent reduction in tumor volume compared to a subject administered an alternative treatment; (vi) a reduction in the dosage required to achieve an equivalent expected rate of survival of a subject compared to a subject administered an alternative treatment; (vii) a reduction in the likelihood of a recurrence of the sarcoma in a subject compared to a subject administered an alternative treatment; (viii) a reduction in the likelihood of a metastasis occurring in a subject compared to a subject administered an alternative treatment; (ix) a reduction in the time required to be at a medical facility for treatment compared to a subject administered an alternative treatment; (x) a reduction in the cost associated with treatment compared to a subject administered an alternative treatment; as well as any other improved outcome not expressly recited that would be readily envisaged by one skilled in the art.

In certain embodiments, a NOS inhibitor composition, as described herein, may be used to treat, ameliorate, and/or reduce the severity of a sarcoma or one or more symptoms thereof, wherein the NOS inhibitor composition comprises at least one chemical NOS inhibitor and at least one biological NOS inhibitor. In some embodiments, the at least one chemical NOS inhibitor may be administered concurrently with the at least one biological NOS inhibitor. In certain embodiments, the at least one chemical NOS inhibitor may be administered prior to the at least one biological NOS inhibitor. In some embodiments, the at least one chemical NOS inhibitor may be administered subsequently to the at least one biological NOS inhibitor. Treatment, amelioration, and/or reduction in the severity of a sarcoma or one or more symptoms thereof may be characterized by an improved outcome compared an alternative treatment. As used herein, the term “alternative treatment,” may define one or more of a conventional therapy, as described herein, a NOS inhibitor composition comprising a single chemical NOS inhibitor, as described herein, and a NOS inhibitor composition comprising a single biological NOS inhibitor, as described herein. For purposes of the disclosure, an “improved outcome” may define any one of the following: (i) a larger reduction in tumor volume and/or mass compared an alternative treatment; (ii) an improvement in the expected rate of survival of a subject, at, for example, 1 month, 3 months, 6 months, 9 months, 1 year, 2 years, 3 years, 5 years, 10 years, or more, compared to the expected rate of survival of a subject administered an alternative treatment; (iii) a reduction in the prevalence of side effects resulting from the NOS inhibitor composition comprising at least one chemical NOS inhibitor and at least one biological NOS inhibitor compared to a subject administered an alternative treatment; (iv) a reduction in the severity of side effects resulting from the NOS inhibitor composition comprising at least one chemical NOS inhibitor and at least one biological NOS inhibitor compared to a subject administered an alternative treatment; (v) a reduction in the dosage required to achieve an equivalent reduction in tumor volume compared to a subject administered an alternative treatment; (vi) a reduction in the dosage required to achieve an equivalent expected rate of survival of a subject compared to a subject administered an alternative treatment; (vii) a reduction in the likelihood of a recurrence of the sarcoma in a subject compared to a subject administered an alternative treatment; (viii) a reduction in the likelihood of a metastasis occurring in a subject compared to a subject administered an alternative treatment; (ix) a reduction in the time required to be at a medical facility for treatment compared to a subject administered an alternative treatment; (x) a reduction in the cost associated with treatment compared to a subject administered an alternative treatment; as well as any other improved outcome not expressly recited that would be readily envisaged by one skilled in the art.

In some embodiments, a NOS inhibitor composition, as described herein, may be combined with a conventional therapy, as described herein, to treat, ameliorate, and/or reduce the severity of a sarcoma or one or more symptoms thereof. In certain embodiments, the NOS inhibitor composition may be administered concurrently with the conventional therapy. In some embodiments, the NOS inhibitor composition may be administered prior to the conventional therapy. In certain embodiments, the NOS inhibitor composition may be administered subsequently to the conventional therapy. Treatment, amelioration, and/or reduction in the severity of a sarcoma or one or more symptoms thereof may be characterized by an improved outcome compared an alternative treatment. As used herein, the term “alternative treatment,” may define one or more of a conventional therapy, as described herein, a NOS inhibitor composition comprising a single chemical NOS inhibitor, as described herein, and a NOS inhibitor composition comprising a single biological NOS inhibitor, as described herein. For purposes of the disclosure, an “improved outcome” may define any one of the following: (i) a larger reduction in tumor volume and/or mass compared an alternative treatment; (ii) an improvement in the expected rate of survival of a subject, at, for example, 1 month, 3 months, 6 months, 9 months, 1 year, 2 years, 3 years, 5 years, 10 years, or more, compared to the expected rate of survival of a subject administered an alternative treatment; (iii) a reduction in the prevalence of side effects resulting from the NOS inhibitor composition and conventional therapy compared to a subject administered an alternative treatment; (iv) a reduction in the severity of side effects resulting from the NOS inhibitor composition and conventional therapy compared to a subject administered an alternative treatment; (v) a reduction in the dosage required to achieve an equivalent reduction in tumor volume compared to a subject administered an alternative treatment; (vi) a reduction in the dosage required to achieve an equivalent expected rate of survival of a subject compared to a subject administered an alternative treatment; (vii) a reduction in the likelihood of a recurrence of the sarcoma in a subject compared to a subject administered an alternative treatment; (viii) a reduction in the likelihood of a metastasis occurring in a subject compared to a subject administered an alternative treatment; (ix) a reduction in the time required to be at a medical facility for treatment compared to a subject administered an alternative treatment; (x) a reduction in the cost associated with treatment compared to a subject administered an alternative treatment; as well as any other improved outcome not expressly recited that would be readily envisaged by one skilled in the art.

In some embodiments, a “conventional therapy,” as that term is used with respect to combined administration with a NOS inhibitor composition, may refer to a conventional chemotherapy. For purposes of the disclosure, a “conventional chemotherapy,” as described herein may be a compound selected from the group consisting of abemaciclib, abiraterone acetate, acalabrutinib, adagrasib, afatinib dimaleate, albumin-bound paclitaxel, albumin-bound rapamycin (Sirolimus Protein-Bound Particles), alectinib, alpelisib, amifostine, aminolevulinic acid hydrochloride, anastrozole, apalutamide, aprepitant, arsenic trioxide, asciminib hydrochloride, avapritinib, axitinib, azacytidine, belinostat, belzutifan, bendamustine hydrochloride, bexarotene, bicalutamide, binimetinib, bleomycin, bleomycin sulfate, bortezomib, bosutinib, brigatinib, busulfan, cabazitaxel, cabozantinib-S-malate, capecitabine, capmatinib hydrochloride, carboplatin, carfilzomib, carmustine, cedazuridine, ceritinib, chlorambucil, cisplatin, cladribine, clofarabine, cobimetinib fumarate, copanlisib hydrochloride, crizotinib, cyclophosphamide, cytarabine, dabrafenib mesylate, dacarbazine, dacomitinib, dactinomycin, darolutamide, dasatinib, daunorubicin hydrochloride, decitabine, defibrotide sodium, degarelix, dexamethasone, dexrazoxane hydrochloride, docetaxel, doxorubicin hydrochloride, duvelisib, elacestrant dihydrochloride, eltrombopag olamine, enasidenib mesylate, encorafenib, entrectinib, enzalutamide, epirubicin hydrochloride, erdafitinib, eribulin mesylate, erlotinib hydrochloride, etoposide, etoposide phosphate, everolimus, exemestane, fedratinib hydrochloride, fludarabine phosphate, fluorouracil, flutamide, folinic acid, fostamatinib disodium, fulvestrant, futibatinib, gefitinib, gemcitabine hydrochloride, gilteritinib fumarate, glasdegib maleate, goserelin acetate, granisetron, granisetron hydrochloride, hydroxyurea, ibrutinib, idarubicin hydrochloride, idelalisib, ifosfamide, imatinib mesylate, imiquimod, infigratinib phosphate, iobenguane I-131, irinotecan hydrochloride, ivosidenib, ixabepilone, ixazomib citrate, lanreotide acetate, lapatinib ditosylate, larotrectinib sulfate, lenalidomide, lenvatinib mesylate, letrozole, leucovorin calcium, leuprolide acetate, liposomal cytarabine, liposomal doxorubicin hydrochloride, liposomal irinotecan hydrochloride, lomustine, lorlatinib, lurbinectedin, Lu-177 dotatate, Lu-177 vipivotide tetraxetan, mechlorethamine hydrochloride, megestrol acetate, melphalan, melphalan hydrochloride, mercaptopurine, mesna, methotrexate, methotrexate sodium, methylnaltrexone bromide, midostaurin, mitomycin, mitoxantrone hydrochloride, mobocertinib succinate, nelarabine, neratinib maleate, netupitant hydrochloride, nilutamide, nilotinib, niraparib tosylate monohydrate, olaparib, olutasidenib, omacetaxine mepesuccinate, ondansetron hydrochloride, osimertinib mesylate, oxaliplatin, paclitaxel, pacritinib citrate, palbociclib, palonosetron hydrochloride, pamidronate disodium, pazopanib hydrochloride, pemetrexed disodium, pemigatinib, pexidartinib hydrochloride, pirtobrutinib, plerixafor, pomalidomide, ponatinib hydrochloride, pralatrexate, pralsetinib, prednisone, procarbazine hydrochloride, propranolol hydrochloride, Ra-223 dichloride, raloxifene hydrochloride, regorafenib, relugolix, ribociclib, ripretinib, rolapitant hydrochloride, romidepsin, rucaparib camsylate, ruxolitinib phosphate, selinexor, selpercatinib, selumetinib sulfate, sonidegib, sorafenib tosylate, sotorasib, sunitinib malate, talazoparib tosylate, talc, tamoxifen citrate, tazemetostat hydrobromide, temozolomide, temsirolimus, tepotinib hydrochloride, thalidomide, thioguanine, thiotepa, tipiracil hydrochloride, tivozanib hydrochloride, topotecan hydrochloride, toremifene, trabectedin, trametinib dimethyl sulfoxide, tretinoin, trifluridine hydrochloride, tucatinib, uridine triacetate, valrubicin, vandetanib, vemurafenib, venetoclax, vinblastine sulfate, vincristine sulfate, vinorelbine tartrate, vismodegib, vorinostat, zanubrutinib, zoledronic acid, and any combination thereof. One skilled in the art would immediately envisage an appropriate “conventional chemotherapy” based on the particular sarcoma disclosed, as well as characteristics of a sarcoma subject, such as, for example, the subject's sex, age, stage of the sarcoma, genotype of the sarcoma, and the like.

In certain embodiments, a “conventional therapy,” as that term is used with respect to combined administration with a NOS inhibitor composition, may refer to a conventional immunotherapy. For purposes of the disclosure, a “conventional immunotherapy,” as described herein may be an immunotherapy, cell-based therapy, and/or gene-based therapy selected from the group consisting of ado-trastuzumab emtansine, aldesleukin, alemtuzumab, amivantamab-vmjw, asparaginase Erwinia chrysanthemi, asparaginase Erwinia chrysanthemi (recombinant)-rywn, atezolizumab, avelumab, axicabtagene ciloleucel, bevacizumab, blinatumomab, brentuximab vedotin, brexucabtagene autoleucel, calaspargase pegol-mknl, camrelizumab, caplacizumab-yhdp, cemiplimab-rwlc, cetuximab, ciltacabtagene autoleucel, daratumumab, daratumumab and hyaluronidase-fihj, darbepoetin alfa, denileukin diftitox, denosumab, dinutuximab, dostarlimab-gxly, durvalumab, elotuzumab, emapalumab-lzsg, enfortumab vedotin-ejfv, epoetin alfa, fam-trastuzumab deruxtecan-nxki, filgrastim, gemtuzumab ozogamicin, glucarpidase, ibritumomab tiuxetan, idecabtagene vicleucel, inotuzumab ozogamicin, ipilimumab, isatuximab-irfc, lisocabtagene maraleucel, loncastuximab tesirine-lpyl, luspatercept-aamt, margetuximab-cmkb, mirvetuximab soravtansine-gynx, mogamulizumab-kpkc, mosunetuzumab-axgb, moxetumomab pasudotox-tdfk, nadofaragene firadenovec-vncg, naxitamab-gqgk, necitumumab, nivolumab, nivolumab and relatlimab-rmbw, obinutuzumab, ofatumumab, palifermin, panitumumab, pegaspargase, pegfilgrastim, pembrolizumab, pertuzumab, pertuzumab, trastuzumab, and hyaluronidase-zzxf, polatuzumab vedotin-piiq, ramucirumab, rasburicase, ravulizumab-cwvz, recombinant HPV bivalent vaccine, recombinant HPV nonavalent vaccine, recombinant HPV quadrivalent vaccine, recombinant interferon alfa-2b, rituximab, rituximab and hyaluronidase human, romiplostim, ropeginterferon alfa-2b-njft, sacituzumab govitecan-hziy, siltuximab, sintilimab, sipuleucel-T, tafasitamab-cxix, tagraxofusp-erzs, talimogene laherparepvec, tebentafusp-tebn, teclistamab-cqyv, tisagenlecleucel, tislelizumab, tisotumab vedotin-tftv, tocilizumab, toripalimab, trastuzumab, trastuzumab and hyaluronidase-oysk, tremelimumab-actl, ziv-aflibercept, and any combination thereof. One skilled in the art would immediately envisage an appropriate “conventional immunotherapy” based on the particular sarcoma disclosed, as well as characteristics of a sarcoma subject, such as, for example, the subject's sex, age, stage of the sarcoma, genotype of the sarcoma, and the like.

In certain embodiments, a NOS inhibitor composition, as described herein, may be administered as a suitable dosage form, as described herein, prior to, or alternatively, following a surgery to remove or resect a sarcoma.

In some embodiments, a NOS inhibitor composition, as described herein, may be administered as a suitable dosage form, as described herein, to restore and/or maintain healthy and/or optimal expression levels of one or more antigens, as described herein, such as, for example, CD15, CD44, CD70, CD133, as well as any CD antigen that has an abnormal expression in a sarcoma disclosed herein that would be readily envisaged by one skilled in the art, in a subject.

In certain embodiments, the subject may have a sarcoma or one or more symptoms thereof. As used herein, the terms “healthy” and “optimal” refer to expression levels of one or more antigens that would be typical of an equivalent subject that does not have a sarcoma.

In certain embodiments, a NOS inhibitor composition, as described herein, may be administered as a suitable dosage form, as described herein, to restore and/or maintain healthy and/or optimal expression levels of one or more proteins, as described herein, such as, for example, ALDH1A3, mTOR, p-mTOR, TSC-2, AKT, pAKT, RPS6, p-RPS6, Syp, VEGF, PIGF, as well as any protein or enzyme that has an abnormal expression in a sarcoma disclosed herein that would be readily envisaged by one skilled in the art, in a subject. In certain embodiments, the subject may have a sarcoma or one or more symptoms thereof. As used herein, the terms “healthy” and “optimal” refer to expression levels of one or more proteins that would be typical of an equivalent subject that does not have a sarcoma.

In some embodiments, a NOS inhibitor composition, as described herein, may be administered as a suitable dosage form, as described herein, to restore and/or maintain healthy and/or optimal expression levels of one or more NOS isoforms, as described herein, such as, for example, nNOS, iNOS, and eNOS, in a subject. In certain embodiments, the subject may have a sarcoma or one or more symptoms thereof. As used herein, the terms “healthy” and “optimal” refer to expression levels of one or more NOS isoforms that would be typical of an equivalent subject that does not have a sarcoma.

In certain embodiments, a NOS inhibitor composition, as described herein, may be administered as a suitable dosage form, as described herein, to restore and/or maintain healthy and/or optimal levels of one or more NO-indicators, as described herein, such as, for example, 3-Ntyr, NADPH-diaphorase, total nitrite concentration, total nitrate concentration, total nitrite and nitrate concentration, as well as any NO-indicator that has an elevated level in a sarcoma disclosed herein that would be readily envisaged by one skilled in the art, in a subject. In certain embodiments, the subject may have a sarcoma or one or more symptoms thereof. As used herein, the terms “healthy” and “optimal” refer to levels of one or more NO-indicators that would be typical of an equivalent subject that does not have sarcoma.

In certain embodiments, a NOS inhibitor composition, as described herein, may be administered as a suitable dosage form, as described herein, to treat, ameliorate, and/or reduce the severity of an ovarian cancer or one or more symptoms thereof in a subject (“ovarian cancer subject”). As used herein, the term “ovarian cancer” refers to a cancer that occurs and/or originates in one or more ovary, fallopian tube, or ovary and fallopian tube.

In some embodiments, a NOS inhibitor composition comprising 7-NI and/or a salt thereof may be administered as a suitable dosage form, as described herein, to treat, ameliorate, and/or reduce the severity of ovarian cancer or one or more symptoms thereof in an ovarian cancer subject. In certain embodiments, an nNOS inhibitor composition comprising 7-NI and/or a salt thereof may be administered to an ovarian cancer subject to treat, ameliorate, and/or reduce the severity of ovarian cancer or one or more symptoms thereof in the ovarian cancer subject: (i) via a route selected from the group consisting of buccally, inhalation, intraarterially, intraarticularly, intracardially, intracerebroventricularly, intradermally, intramedullary, intramuscularly, intraperitoneally, intrathecally, intravenously, intraventricularly, nasally, ophthalmically, orally, rectally, sublabially, sublingually, subcutaneously, topically, and transdermally; and/or (ii) with a frequency selected from the group consisting of four times daily, three times daily, twice daily, once daily, once every two days, once every three days, once every four days, once every five days, once every six days, once a week, once every eight days, once every nine days, once every ten days, once every two weeks, once every three weeks, and once a month; and/or (iii) at a dosage (based on subject weight) selected from the group consisting of 0.1 μg/kg, 0.5 μg/kg, 1 μg/kg, 1.5 μg/kg, 2.0 μg/kg, 2.5 μg/kg, 3.0 μg/kg, 3.5 μg/kg, 4.0 μg/kg, 4.5 μg/kg, 5.0 μg/kg, 10 μg/kg, 15 μg/kg, 20 μg/kg, 25 μg/kg, 30 μg/kg, 35 μg/kg, 40 μg/kg, 45 μg/kg, 50 μg/kg, 55 μg/kg, 60 μg/kg, 65 μg/kg, 70 μg/kg, 75 μg/kg, 80 μg/kg, 85 μg/kg, 90 μg/kg, 95 μg/kg, 100 μg/kg, 150 μg/kg, 200 μg/kg, 250 μg/kg, 300 μg/kg, 350 μg/kg, 400 μg/kg, 450 μg/kg, 500 μg/kg, 550 μg/kg, 600 μg/kg, 650 μg/kg, 700 μg/kg, 750 μg/kg, 800 μg/kg, 850 μg/kg, 900 μg/kg, 1 mg/kg, 1.5 mg/kg, 2.0 mg/kg, 2.5 mg/kg, 3 mg/kg, 3.5 mg/kg, 4.0 mg/kg, 4.5 mg/kg, 5 mg/kg, 5.5 mg/kg, 6 mg/kg, 6.5 mg/kg, 7 mg/kg, 7.5 mg/kg, 8 mg/kg, 8.5 mg/kg, 9 mg/kg, 9.5 mg/kg, 10 mg/kg 10.5 mg/kg, 11 mg/kg, 11.5 mg/kg, 12 mg/kg, 12.5 mg/kg, 13 mg/kg, 13.5 mg/kg, 14 mg/kg, 14.5 mg/kg, 15 mg/kg, 16 mg/kg, 17 mg/kg, 18 mg/kg, 19 mg/kg, 20 mg/kg, 21 mg/kg, 22 mg/kg, 23 mg/kg, 24 mg/kg, 25 mg/kg, 30 mg/kg, 35 mg/kg, 40 mg/kg, 45 mg/kg, 50 mg/kg, 55 mg/kg, 60 mg/kg, 65 mg/kg, 70 mg/kg, 75 mg/kg, 80 mg/kg, 85 mg/kg, 90 mg/kg, 95 mg/kg, 100 mg/kg, 105 mg/kg, 110 mg/kg, 115 mg/kg, 120 mg/kg, 125 mg/kg, 130 mg/kg, 135 mg/kg, 140 mg/kg, 145 mg/kg, 150 mg/kg, 155 mg/kg, 160 mg/kg, 165 mg/kg, 170 mg/kg, 175 mg/kg, 180 mg/kg, 185 mg/kg, 190 mg/kg, 195 mg/kg, 200 mg/kg, and any fraction or integer in between any two of the preceding amounts.

In certain embodiments, a NOS inhibitor composition comprising 3-Br-7-NI and/or a salt thereof may be administered as a suitable dosage form, as described herein, to treat, ameliorate, and/or reduce the severity of ovarian cancer or one or more symptoms thereof in an ovarian cancer subject. In some embodiments, an nNOS inhibitor composition comprising 3-Br-7-NI and/or a salt thereof may be administered to an ovarian cancer subject to treat, ameliorate, and/or reduce the severity of ovarian cancer or one or more symptoms thereof in the ovarian cancer subject: (i) via a route selected from the group consisting of buccally, inhalation, intraarterially, intraarticularly, intracardially, intracerebroventricularly, intradermally, intramedullary, intramuscularly, intraperitoneally, intrathecally, intravenously, intraventricularly, nasally, ophthalmically, orally, rectally, sublabially, sublingually, subcutaneously, topically, and transdermally; and/or (ii) with a frequency selected from the group consisting of four times daily, three times daily, twice daily, once daily, once every two days, once every three days, once every four days, once every five days, once every six days, once a week, once every eight days, once every nine days, once every ten days, once every two weeks, once every three weeks, and once a month; and/or (iii) at a dosage (based on subject weight) selected from the group consisting of 0.1 μg/kg, 0.5 μg/kg, 1 μg/kg, 1.5 μg/kg, 2.0 μg/kg, 2.5 μg/kg, 3.0 μg/kg, 3.5 μg/kg, 4.0 μg/kg, 4.5 μg/kg, 5.0 μg/kg, 10 μg/kg, 15 μg/kg, 20 μg/kg, 25 μg/kg, 30 μg/kg, 35 μg/kg, 40 μg/kg, 45 μg/kg, 50 μg/kg, 55 μg/kg, 60 μg/kg, 65 μg/kg, 70 μg/kg, 75 μg/kg, 80 μg/kg, 85 μg/kg, 90 μg/kg, 95 μg/kg, 100 μg/kg, 150 μg/kg, 200 μg/kg, 250 μg/kg, 300 μg/kg, 350 μg/kg, 400 μg/kg, 450 μg/kg, 500 μg/kg, 550 μg/kg, 600 μg/kg, 650 μg/kg, 700 μg/kg, 750 μg/kg, 800 μg/kg, 850 μg/kg, 900 μg/kg, 1 mg/kg, 1.5 mg/kg, 2.0 mg/kg, 2.5 mg/kg, 3 mg/kg, 3.5 mg/kg, 4.0 mg/kg, 4.5 mg/kg, 5 mg/kg, 5.5 mg/kg, 6 mg/kg, 6.5 mg/kg, 7 mg/kg, 7.5 mg/kg, 8 mg/kg, 8.5 mg/kg, 9 mg/kg, 9.5 mg/kg, 10 mg/kg 10.5 mg/kg, 11 mg/kg, 11.5 mg/kg, 12 mg/kg, 12.5 mg/kg, 13 mg/kg, 13.5 mg/kg, 14 mg/kg, 14.5 mg/kg, 15 mg/kg, 16 mg/kg, 17 mg/kg, 18 mg/kg, 19 mg/kg, 20 mg/kg, 21 mg/kg, 22 mg/kg, 23 mg/kg, 24 mg/kg, 25 mg/kg, 30 mg/kg, 35 mg/kg, 40 mg/kg, 45 mg/kg, 50 mg/kg, 55 mg/kg, 60 mg/kg, 65 mg/kg, 70 mg/kg, 75 mg/kg, 80 mg/kg, 85 mg/kg, 90 mg/kg, 95 mg/kg, 100 mg/kg, 105 mg/kg, 110 mg/kg, 115 mg/kg, 120 mg/kg, 125 mg/kg, 130 mg/kg, 135 mg/kg, 140 mg/kg, 145 mg/kg, 150 mg/kg, 155 mg/kg, 160 mg/kg, 165 mg/kg, 170 mg/kg, 175 mg/kg, 180 mg/kg, 185 mg/kg, 190 mg/kg, 195 mg/kg, 200 mg/kg, and any fraction or integer in between any two of the preceding amounts.

In some embodiments, a NOS inhibitor composition comprising L-NPA and/or a salt thereof, such as, for example, L-NPA hydrochloride, may be administered as a suitable dosage form, as described herein, to treat, ameliorate, and/or reduce the severity of ovarian cancer or one or more symptoms thereof in an ovarian cancer subject. In certain embodiments, an nNOS inhibitor composition comprising L-NPA and/or a salt thereof, such as, for example, L-NPA hydrochloride may be administered to an ovarian cancer subject to treat, ameliorate, and/or reduce the severity of ovarian cancer or one or more symptoms thereof in the ovarian cancer subject: (i) via a route selected from the group consisting of buccally, inhalation, intraarterially, intraarticularly, intracardially, intracerebroventricularly, intradermally, intramedullary, intramuscularly, intraperitoneally, intrathecally, intravenously, intraventricularly, nasally, ophthalmically, orally, rectally, sublabially, sublingually, subcutaneously, topically, and transdermally; and/or (ii) with a frequency selected from the group consisting of four times daily, three times daily, twice daily, once daily, once every two days, once every three days, once every four days, once every five days, once every six days, once a week, once every eight days, once every nine days, once every ten days, once every two weeks, once every three weeks, and once a month; and/or (iii) at a dosage (based on subject weight) selected from the group consisting of 0.1 μg/kg, 0.5 μg/kg, 1 μg/kg, 1.5 μg/kg, 2.0 μg/kg, 2.5 μg/kg, 3.0 μg/kg, 3.5 μg/kg, 4.0 μg/kg, 4.5 μg/kg, 5.0 μg/kg, 10 μg/kg, 15 μg/kg, 20 μg/kg, 25 μg/kg, 30 μg/kg, 35 μg/kg, 40 μg/kg, 45 μg/kg, 50 μg/kg, 55 μg/kg, 60 μg/kg, 65 μg/kg, 70 μg/kg, 75 μg/kg, 80 μg/kg, 85 μg/kg, 90 μg/kg, 95 μg/kg, 100 μg/kg, 150 μg/kg, 200 μg/kg, 250 μg/kg, 300 μg/kg, 350 μg/kg, 400 μg/kg, 450 μg/kg, 500 μg/kg, 550 μg/kg, 600 μg/kg, 650 μg/kg, 700 μg/kg, 750 μg/kg, 800 μg/kg, 850 μg/kg, 900 μg/kg, 1 mg/kg, 1.5 mg/kg, 2.0 mg/kg, 2.5 mg/kg, 3 mg/kg, 3.5 mg/kg, 4.0 mg/kg, 4.5 mg/kg, 5 mg/kg, 5.5 mg/kg, 6 mg/kg, 6.5 mg/kg, 7 mg/kg, 7.5 mg/kg, 8 mg/kg, 8.5 mg/kg, 9 mg/kg, 9.5 mg/kg, 10 mg/kg 10.5 mg/kg, 11 mg/kg, 11.5 mg/kg, 12 mg/kg, 12.5 mg/kg, 13 mg/kg, 13.5 mg/kg, 14 mg/kg, 14.5 mg/kg, 15 mg/kg, 16 mg/kg, 17 mg/kg, 18 mg/kg, 19 mg/kg, 20 mg/kg, 21 mg/kg, 22 mg/kg, 23 mg/kg, 24 mg/kg, 25 mg/kg, 30 mg/kg, 35 mg/kg, 40 mg/kg, 45 mg/kg, 50 mg/kg, 55 mg/kg, 60 mg/kg, 65 mg/kg, 70 mg/kg, 75 mg/kg, 80 mg/kg, 85 mg/kg, 90 mg/kg, 95 mg/kg, 100 mg/kg, 105 mg/kg, 110 mg/kg, 115 mg/kg, 120 mg/kg, 125 mg/kg, 130 mg/kg, 135 mg/kg, 140 mg/kg, 145 mg/kg, 150 mg/kg, 155 mg/kg, 160 mg/kg, 165 mg/kg, 170 mg/kg, 175 mg/kg, 180 mg/kg, 185 mg/kg, 190 mg/kg, 195 mg/kg, 200 mg/kg, and any fraction or integer in between any two of the preceding amounts.

In certain embodiments, a NOS inhibitor composition comprising L-NIO and/or a salt thereof, such as, for example, L-NIO dihydrochloride, may be administered as a suitable dosage form, as described herein, to treat, ameliorate, and/or reduce the severity of ovarian cancer or one or more symptoms thereof in an ovarian cancer subject. In some embodiments, an nNOS inhibitor composition comprising L-NIO and/or a salt thereof, such as, for example, L-NIO dihydrochloride, may be administered to an ovarian cancer subject to treat, ameliorate, and/or reduce the severity of ovarian cancer or one or more symptoms thereof in the ovarian cancer subject: (i) via a route selected from the group consisting of buccally, inhalation, intraarterially, intraarticularly, intracardially, intracerebroventricularly, intradermally, intramedullary, intramuscularly, intraperitoneally, intrathecally, intravenously, intraventricularly, nasally, ophthalmically, orally, rectally, sublabially, sublingually, subcutaneously, topically, and transdermally; and/or (ii) with a frequency selected from the group consisting of four times daily, three times daily, twice daily, once daily, once every two days, once every three days, once every four days, once every five days, once every six days, once a week, once every eight days, once every nine days, once every ten days, once every two weeks, once every three weeks, and once a month; and/or (iii) at a dosage (based on subject weight) selected from the group consisting of 0.1 μg/kg, 0.5 μg/kg, 1 μg/kg, 1.5 μg/kg, 2.0 μg/kg, 2.5 μg/kg, 3.0 μg/kg, 3.5 μg/kg, 4.0 μg/kg, 4.5 μg/kg, 5.0 μg/kg, 10 μg/kg, 15 μg/kg, 20 μg/kg, 25 μg/kg, 30 μg/kg, 35 μg/kg, 40 μg/kg, 45 μg/kg, 50 μg/kg, 55 μg/kg, 60 μg/kg, 65 μg/kg, 70 μg/kg, 75 μg/kg, 80 μg/kg, 85 μg/kg, 90 μg/kg, 95 μg/kg, 100 μg/kg, 150 μg/kg, 200 μg/kg, 250 μg/kg, 300 μg/kg, 350 μg/kg, 400 μg/kg, 450 μg/kg, 500 μg/kg, 550 μg/kg, 600 μg/kg, 650 μg/kg, 700 μg/kg, 750 μg/kg, 800 μg/kg, 850 μg/kg, 900 μg/kg, 1 mg/kg, 1.5 mg/kg, 2.0 mg/kg, 2.5 mg/kg, 3 mg/kg, 3.5 mg/kg, 4.0 mg/kg, 4.5 mg/kg, 5 mg/kg, 5.5 mg/kg, 6 mg/kg, 6.5 mg/kg, 7 mg/kg, 7.5 mg/kg, 8 mg/kg, 8.5 mg/kg, 9 mg/kg, 9.5 mg/kg, 10 mg/kg 10.5 mg/kg, 11 mg/kg, 11.5 mg/kg, 12 mg/kg, 12.5 mg/kg, 13 mg/kg, 13.5 mg/kg, 14 mg/kg, 14.5 mg/kg, 15 mg/kg, 16 mg/kg, 17 mg/kg, 18 mg/kg, 19 mg/kg, 20 mg/kg, 21 mg/kg, 22 mg/kg, 23 mg/kg, 24 mg/kg, 25 mg/kg, 30 mg/kg, 35 mg/kg, 40 mg/kg, 45 mg/kg, 50 mg/kg, 55 mg/kg, 60 mg/kg, 65 mg/kg, 70 mg/kg, 75 mg/kg, 80 mg/kg, 85 mg/kg, 90 mg/kg, 95 mg/kg, 100 mg/kg, 105 mg/kg, 110 mg/kg, 115 mg/kg, 120 mg/kg, 125 mg/kg, 130 mg/kg, 135 mg/kg, 140 mg/kg, 145 mg/kg, 150 mg/kg, 155 mg/kg, 160 mg/kg, 165 mg/kg, 170 mg/kg, 175 mg/kg, 180 mg/kg, 185 mg/kg, 190 mg/kg, 195 mg/kg, 200 mg/kg, and any fraction or integer in between any two of the preceding amounts.

In some embodiments, a NOS inhibitor composition comprising ARL-17477 and/or a salt thereof, such as, for example, ARL-17477 dihydrochloride, may be administered as a suitable dosage form, as described herein, to treat, ameliorate, and/or reduce the severity of ovarian cancer or one or more symptoms thereof in an ovarian cancer subject. In certain embodiments, an nNOS inhibitor composition comprising ARL-17477 and/or a salt thereof, such as, for example, ARL-17477 dihydrochloride, may be administered to an ovarian cancer subject to treat, ameliorate, and/or reduce the severity of ovarian cancer or one or more symptoms thereof in the ovarian cancer subject: (i) via a route selected from the group consisting of buccally, inhalation, intraarterially, intraarticularly, intracardially, intracerebroventricularly, intradermally, intramedullary, intramuscularly, intraperitoneally, intrathecally, intravenously, intraventricularly, nasally, ophthalmically, orally, rectally, sublabially, sublingually, subcutaneously, topically, and transdermally; and/or (ii) with a frequency selected from the group consisting of four times daily, three times daily, twice daily, once daily, once every two days, once every three days, once every four days, once every five days, once every six days, once a week, once every eight days, once every nine days, once every ten days, once every two weeks, once every three weeks, and once a month; and/or (iii) at a dosage (based on subject weight) selected from the group consisting of 0.1 μg/kg, 0.5 μg/kg, 1 μg/kg, 1.5 μg/kg, 2.0 μg/kg, 2.5 μg/kg, 3.0 μg/kg, 3.5 μg/kg, 4.0 μg/kg, 4.5 μg/kg, 5.0 μg/kg, 10 μg/kg, 15 μg/kg, 20 μg/kg, 25 μg/kg, 30 μg/kg, 35 μg/kg, 40 μg/kg, 45 μg/kg, 50 μg/kg, 55 μg/kg, 60 μg/kg, 65 μg/kg, 70 μg/kg, 75 μg/kg, 80 μg/kg, 85 μg/kg, 90 μg/kg, 95 μg/kg, 100 μg/kg, 150 μg/kg, 200 μg/kg, 250 μg/kg, 300 μg/kg, 350 μg/kg, 400 μg/kg, 450 μg/kg, 500 μg/kg, 550 μg/kg, 600 μg/kg, 650 μg/kg, 700 μg/kg, 750 μg/kg, 800 μg/kg, 850 μg/kg, 900 μg/kg, 1 mg/kg, 1.5 mg/kg, 2.0 mg/kg, 2.5 mg/kg, 3 mg/kg, 3.5 mg/kg, 4.0 mg/kg, 4.5 mg/kg, 5 mg/kg, 5.5 mg/kg, 6 mg/kg, 6.5 mg/kg, 7 mg/kg, 7.5 mg/kg, 8 mg/kg, 8.5 mg/kg, 9 mg/kg, 9.5 mg/kg, 10 mg/kg 10.5 mg/kg, 11 mg/kg, 11.5 mg/kg, 12 mg/kg, 12.5 mg/kg, 13 mg/kg, 13.5 mg/kg, 14 mg/kg, 14.5 mg/kg, 15 mg/kg, 16 mg/kg, 17 mg/kg, 18 mg/kg, 19 mg/kg, 20 mg/kg, 21 mg/kg, 22 mg/kg, 23 mg/kg, 24 mg/kg, 25 mg/kg, 30 mg/kg, 35 mg/kg, 40 mg/kg, 45 mg/kg, 50 mg/kg, 55 mg/kg, 60 mg/kg, 65 mg/kg, 70 mg/kg, 75 mg/kg, 80 mg/kg, 85 mg/kg, 90 mg/kg, 95 mg/kg, 100 mg/kg, 105 mg/kg, 110 mg/kg, 115 mg/kg, 120 mg/kg, 125 mg/kg, 130 mg/kg, 135 mg/kg, 140 mg/kg, 145 mg/kg, 150 mg/kg, 155 mg/kg, 160 mg/kg, 165 mg/kg, 170 mg/kg, 175 mg/kg, 180 mg/kg, 185 mg/kg, 190 mg/kg, 195 mg/kg, 200 mg/kg, and any fraction or integer in between any two of the preceding amounts.

In certain embodiments, a NOS inhibitor composition comprising L-NNA and/or a salt thereof may be administered as a suitable dosage form, as described herein, to treat, ameliorate, and/or reduce the severity of ovarian cancer or one or more symptoms thereof in an ovarian cancer subject. In some embodiments, an nNOS inhibitor composition comprising L-NNA and/or a salt thereof may be administered to an ovarian cancer subject to treat, ameliorate, and/or reduce the severity of ovarian cancer or one or more symptoms thereof in the ovarian cancer subject: (i) via a route selected from the group consisting of buccally, inhalation, intraarterially, intraarticularly, intracardially, intracerebroventricularly, intradermally, intramedullary, intramuscularly, intraperitoneally, intrathecally, intravenously, intraventricularly, nasally, ophthalmically, orally, rectally, sublabially, sublingually, subcutaneously, topically, and transdermally; and/or (ii) with a frequency selected from the group consisting of four times daily, three times daily, twice daily, once daily, once every two days, once every three days, once every four days, once every five days, once every six days, once a week, once every eight days, once every nine days, once every ten days, once every two weeks, once every three weeks, and once a month; and/or (iii) at a dosage (based on subject weight) selected from the group consisting of 0.1 μg/kg, 0.5 μg/kg, 1 μg/kg, 1.5 μg/kg, 2.0 μg/kg, 2.5 μg/kg, 3.0 μg/kg, 3.5 μg/kg, 4.0 μg/kg, 4.5 μg/kg, 5.0 μg/kg, 10 μg/kg, 15 μg/kg, 20 μg/kg, 25 μg/kg, 30 μg/kg, 35 μg/kg, 40 μg/kg, 45 μg/kg, 50 μg/kg, 55 μg/kg, 60 μg/kg, 65 μg/kg, 70 μg/kg, 75 μg/kg, 80 μg/kg, 85 μg/kg, 90 μg/kg, 95 μg/kg, 100 μg/kg, 150 μg/kg, 200 μg/kg, 250 μg/kg, 300 μg/kg, 350 μg/kg, 400 μg/kg, 450 μg/kg, 500 μg/kg, 550 μg/kg, 600 μg/kg, 650 μg/kg, 700 μg/kg, 750 μg/kg, 800 μg/kg, 850 μg/kg, 900 μg/kg, 1 mg/kg, 1.5 mg/kg, 2.0 mg/kg, 2.5 mg/kg, 3 mg/kg, 3.5 mg/kg, 4.0 mg/kg, 4.5 mg/kg, 5 mg/kg, 5.5 mg/kg, 6 mg/kg, 6.5 mg/kg, 7 mg/kg, 7.5 mg/kg, 8 mg/kg, 8.5 mg/kg, 9 mg/kg, 9.5 mg/kg, 10 mg/kg 10.5 mg/kg, 11 mg/kg, 11.5 mg/kg, 12 mg/kg, 12.5 mg/kg, 13 mg/kg, 13.5 mg/kg, 14 mg/kg, 14.5 mg/kg, 15 mg/kg, 16 mg/kg, 17 mg/kg, 18 mg/kg, 19 mg/kg, 20 mg/kg, 21 mg/kg, 22 mg/kg, 23 mg/kg, 24 mg/kg, 25 mg/kg, 30 mg/kg, 35 mg/kg, 40 mg/kg, 45 mg/kg, 50 mg/kg, 55 mg/kg, 60 mg/kg, 65 mg/kg, 70 mg/kg, 75 mg/kg, 80 mg/kg, 85 mg/kg, 90 mg/kg, 95 mg/kg, 100 mg/kg, 105 mg/kg, 110 mg/kg, 115 mg/kg, 120 mg/kg, 125 mg/kg, 130 mg/kg, 135 mg/kg, 140 mg/kg, 145 mg/kg, 150 mg/kg, 155 mg/kg, 160 mg/kg, 165 mg/kg, 170 mg/kg, 175 mg/kg, 180 mg/kg, 185 mg/kg, 190 mg/kg, 195 mg/kg, 200 mg/kg, and any fraction or integer in between any two of the preceding amounts.

In some embodiments, a NOS inhibitor composition comprising L-NAME and/or a salt thereof, such as, for example, L-NAME hydrochloride, may be administered as a suitable dosage form, as described herein, to treat, ameliorate, and/or reduce the severity of ovarian cancer or one or more symptoms thereof in an ovarian cancer subject. In certain embodiments, an nNOS inhibitor composition comprising L-NAME and/or a salt thereof, such as, for example, L-NAME hydrochloride, may be administered to an ovarian cancer subject to treat, ameliorate, and/or reduce the severity of ovarian cancer or one or more symptoms thereof in the ovarian cancer subject: (i) via a route selected from the group consisting of buccally, inhalation, intraarterially, intraarticularly, intracardially, intracerebroventricularly, intradermally, intramedullary, intramuscularly, intraperitoneally, intrathecally, intravenously, intraventricularly, nasally, ophthalmically, orally, rectally, sublabially, sublingually, subcutaneously, topically, and transdermally; and/or (ii) with a frequency selected from the group consisting of four times daily, three times daily, twice daily, once daily, once every two days, once every three days, once every four days, once every five days, once every six days, once a week, once every eight days, once every nine days, once every ten days, once every two weeks, once every three weeks, and once a month; and/or (iii) at a dosage (based on subject weight) selected from the group consisting of 0.1 μg/kg, 0.5 μg/kg, 1 μg/kg, 1.5 μg/kg, 2.0 μg/kg, 2.5 μg/kg, 3.0 μg/kg, 3.5 μg/kg, 4.0 μg/kg, 4.5 μg/kg, 5.0 μg/kg, 10 μg/kg, 15 μg/kg, 20 μg/kg, 25 μg/kg, 30 μg/kg, 35 μg/kg, 40 μg/kg, 45 μg/kg, 50 μg/kg, 55 μg/kg, 60 μg/kg, 65 μg/kg, 70 μg/kg, 75 μg/kg, 80 μg/kg, 85 μg/kg, 90 μg/kg, 95 μg/kg, 100 μg/kg, 150 μg/kg, 200 μg/kg, 250 μg/kg, 300 μg/kg, 350 μg/kg, 400 μg/kg, 450 μg/kg, 500 μg/kg, 550 μg/kg, 600 μg/kg, 650 μg/kg, 700 μg/kg, 750 μg/kg, 800 μg/kg, 850 μg/kg, 900 μg/kg, 1 mg/kg, 1.5 mg/kg, 2.0 mg/kg, 2.5 mg/kg, 3 mg/kg, 3.5 mg/kg, 4.0 mg/kg, 4.5 mg/kg, 5 mg/kg, 5.5 mg/kg, 6 mg/kg, 6.5 mg/kg, 7 mg/kg, 7.5 mg/kg, 8 mg/kg, 8.5 mg/kg, 9 mg/kg, 9.5 mg/kg, 10 mg/kg 10.5 mg/kg, 11 mg/kg, 11.5 mg/kg, 12 mg/kg, 12.5 mg/kg, 13 mg/kg, 13.5 mg/kg, 14 mg/kg, 14.5 mg/kg, 15 mg/kg, 16 mg/kg, 17 mg/kg, 18 mg/kg, 19 mg/kg, 20 mg/kg, 21 mg/kg, 22 mg/kg, 23 mg/kg, 24 mg/kg, 25 mg/kg, 30 mg/kg, 35 mg/kg, 40 mg/kg, 45 mg/kg, 50 mg/kg, 55 mg/kg, 60 mg/kg, 65 mg/kg, 70 mg/kg, 75 mg/kg, 80 mg/kg, 85 mg/kg, 90 mg/kg, 95 mg/kg, 100 mg/kg, 105 mg/kg, 110 mg/kg, 115 mg/kg, 120 mg/kg, 125 mg/kg, 130 mg/kg, 135 mg/kg, 140 mg/kg, 145 mg/kg, 150 mg/kg, 155 mg/kg, 160 mg/kg, 165 mg/kg, 170 mg/kg, 175 mg/kg, 180 mg/kg, 185 mg/kg, 190 mg/kg, 195 mg/kg, 200 mg/kg, and any fraction or integer in between any two of the preceding amounts.

In certain embodiments, a NOS inhibitor composition comprising L-NMMA and/or a salt thereof, such as, for example, L-NMMA acetate, may be administered as a suitable dosage form, as described herein, to treat, ameliorate, and/or reduce the severity of ovarian cancer or one or more symptoms thereof in an ovarian cancer subject. In some embodiments, an nNOS inhibitor composition comprising L-NMMA and/or a salt thereof, such as, for example, L-NMMA acetate, may be administered to an ovarian cancer subject to treat, ameliorate, and/or reduce the severity of ovarian cancer or one or more symptoms thereof in the ovarian cancer subject: (i) via a route selected from the group consisting of buccally, inhalation, intraarterially, intraarticularly, intracardially, intracerebroventricularly, intradermally, intramedullary, intramuscularly, intraperitoneally, intrathecally, intravenously, intraventricularly, nasally, ophthalmically, orally, rectally, sublabially, sublingually, subcutaneously, topically, and transdermally; and/or (ii) with a frequency selected from the group consisting of four times daily, three times daily, twice daily, once daily, once every two days, once every three days, once every four days, once every five days, once every six days, once a week, once every eight days, once every nine days, once every ten days, once every two weeks, once every three weeks, and once a month; and/or (iii) at a dosage (based on subject weight) selected from the group consisting of 0.1 μ/kg, 0.5 μ/kg, 1μ/kg, 1.5 μ/kg, 2.0 μ/kg, 2.5 μ/kg, 3.0 μ/kg, 3.5 μ/kg, 4.0 μ/kg, 4.5 μ/kg, 5.0 μ/kg, 10 μ/kg, 15 μ/kg, 20 μ/kg, 25 μ/kg, 30 μ/kg, 35 μ/kg, 40 μ/kg, 45 μ/kg, 50μ/kg, 55 μ/kg, 60 μ/kg, 65 μ/kg, 70 μ/kg, 75 μ/kg, 80 μ/kg, 85 μ/kg, 90 μ/kg, 95 μ/kg, 100 μ/kg, 150 μ/kg, 200 μ/kg, 250 μ/kg, 300 μ/kg, 350 μ/kg, 400 μ/kg, 450 μ/kg, 500 μ/kg, 550 μ/kg, 600 μ/kg, 650 μ/kg, 700 μ/kg, 750 μ/kg, 800 μ/kg, 850 μ/kg, 900 μ/kg, 1 mg/kg, 1.5 mg/kg, 2.0 mg/kg, 2.5 mg/kg, 3 mg/kg, 3.5 mg/kg, 4.0 mg/kg, 4.5 mg/kg, 5 mg/kg, 5.5 mg/kg, 6 mg/kg, 6.5 mg/kg, 7 mg/kg, 7.5 mg/kg, 8 mg/kg, 8.5 mg/kg, 9 mg/kg, 9.5 mg/kg, 10 mg/kg 10.5 mg/kg, 11 mg/kg, 11.5 mg/kg, 12 mg/kg, 12.5 mg/kg, 13 mg/kg, 13.5 mg/kg, 14 mg/kg, 14.5 mg/kg, 15 mg/kg, 16 mg/kg, 17 mg/kg, 18 mg/kg, 19 mg/kg, 20 mg/kg, 21 mg/kg, 22 mg/kg, 23 mg/kg, 24 mg/kg, 25 mg/kg, 30 mg/kg, 35 mg/kg, 40 mg/kg, 45 mg/kg, 50 mg/kg, 55 mg/kg, 60 mg/kg, 65 mg/kg, 70 mg/kg, 75 mg/kg, 80 mg/kg, 85 mg/kg, 90 mg/kg, 95 mg/kg, 100 mg/kg, 105 mg/kg, 110 mg/kg, 115 mg/kg, 120 mg/kg, 125 mg/kg, 130 mg/kg, 135 mg/kg, 140 mg/kg, 145 mg/kg, 150 mg/kg, 155 mg/kg, 160 mg/kg, 165 mg/kg, 170 mg/kg, 175 mg/kg, 180 mg/kg, 185 mg/kg, 190 mg/kg, 195 mg/kg, 200 mg/kg, and any fraction or integer in between any two of the preceding amounts.

In some embodiments, a NOS inhibitor composition comprising GW-274150 and/or a salt thereof, such as, for example, GW-274150 phosphate, may be administered as a suitable dosage form, as described herein, to treat, ameliorate, and/or reduce the severity of ovarian cancer or one or more symptoms thereof in an ovarian cancer subject. In certain embodiments, an nNOS inhibitor composition comprising GW-274150 and/or a salt thereof, such as, for example, GW-274150 phosphate, may be administered to an ovarian cancer subject to treat, ameliorate, and/or reduce the severity of ovarian cancer or one or more symptoms thereof in the ovarian cancer subject: (i) via a route selected from the group consisting of buccally, inhalation, intraarterially, intraarticularly, intracardially, intracerebroventricularly, intradermally, intramedullary, intramuscularly, intraperitoneally, intrathecally, intravenously, intraventricularly, nasally, ophthalmically, orally, rectally, sublabially, sublingually, subcutaneously, topically, and transdermally; and/or (ii) with a frequency selected from the group consisting of four times daily, three times daily, twice daily, once daily, once every two days, once every three days, once every four days, once every five days, once every six days, once a week, once every eight days, once every nine days, once every ten days, once every two weeks, once every three weeks, and once a month; and/or (iii) at a dosage (based on subject weight) selected from the group consisting of 0.1 μg/kg, 0.5 μg/kg, 1 μg/kg, 1.5 μg/kg, 2.0 μg/kg, 2.5 μg/kg, 3.0 μg/kg, 3.5 μg/kg, 4.0 μg/kg, 4.5 μg/kg, 5.0 μg/kg, 10 μg/kg, 15 μg/kg, 20 μg/kg, 25 μg/kg, 30 μg/kg, 35 μg/kg, 40 μg/kg, 45 μg/kg, 50 μg/kg, 55 μg/kg, 60 μg/kg, 65 μg/kg, 70 μg/kg, 75 μg/kg, 80 μg/kg, 85 μg/kg, 90 μg/kg, 95 μg/kg, 100 μg/kg, 150 μg/kg, 200 μg/kg, 250 μg/kg, 300 μg/kg, 350 μg/kg, 400 μg/kg, 450 μg/kg, 500 μg/kg, 550 μg/kg, 600 μg/kg, 650 μg/kg, 700 μg/kg, 750 μg/kg, 800 μg/kg, 850 μg/kg, 900 μg/kg, 1 mg/kg, 1.5 mg/kg, 2.0 mg/kg, 2.5 mg/kg, 3 mg/kg, 3.5 mg/kg, 4.0 mg/kg, 4.5 mg/kg, 5 mg/kg, 5.5 mg/kg, 6 mg/kg, 6.5 mg/kg, 7 mg/kg, 7.5 mg/kg, 8 mg/kg, 8.5 mg/kg, 9 mg/kg, 9.5 mg/kg, 10 mg/kg 10.5 mg/kg, 11 mg/kg, 11.5 mg/kg, 12 mg/kg, 12.5 mg/kg, 13 mg/kg, 13.5 mg/kg, 14 mg/kg, 14.5 mg/kg, 15 mg/kg, 16 mg/kg, 17 mg/kg, 18 mg/kg, 19 mg/kg, 20 mg/kg, 21 mg/kg, 22 mg/kg, 23 mg/kg, 24 mg/kg, 25 mg/kg, 30 mg/kg, 35 mg/kg, 40 mg/kg, 45 mg/kg, 50 mg/kg, 55 mg/kg, 60 mg/kg, 65 mg/kg, 70 mg/kg, 75 mg/kg, 80 mg/kg, 85 mg/kg, 90 mg/kg, 95 mg/kg, 100 mg/kg, 105 mg/kg, 110 mg/kg, 115 mg/kg, 120 mg/kg, 125 mg/kg, 130 mg/kg, 135 mg/kg, 140 mg/kg, 145 mg/kg, 150 mg/kg, 155 mg/kg, 160 mg/kg, 165 mg/kg, 170 mg/kg, 175 mg/kg, 180 mg/kg, 185 mg/kg, 190 mg/kg, 195 mg/kg, 200 mg/kg, and any fraction or integer in between any two of the preceding amounts.

In certain embodiments, a NOS inhibitor composition comprising L-NIL and/or a salt thereof, such as, for example, L-NIL hydrochloride, may be administered as a suitable dosage form, as described herein, to treat, ameliorate, and/or reduce the severity of ovarian cancer or one or more symptoms thereof in an ovarian cancer subject. In some embodiments, an nNOS inhibitor composition comprising L-NIL and/or a salt thereof, such as, for example, L-NIL hydrochloride, may be administered to an ovarian cancer subject to treat, ameliorate, and/or reduce the severity of ovarian cancer or one or more symptoms thereof in the ovarian cancer subject: (i) via a route selected from the group consisting of buccally, inhalation, intraarterially, intraarticularly, intracardially, intracerebroventricularly, intradermally, intramedullary, intramuscularly, intraperitoneally, intrathecally, intravenously, intraventricularly, nasally, ophthalmically, orally, rectally, sublabially, sublingually, subcutaneously, topically, and transdermally; and/or (ii) with a frequency selected from the group consisting of four times daily, three times daily, twice daily, once daily, once every two days, once every three days, once every four days, once every five days, once every six days, once a week, once every eight days, once every nine days, once every ten days, once every two weeks, once every three weeks, and once a month; and/or (iii) at a dosage (based on subject weight) selected from the group consisting of 0.1 μg/kg, 0.5 μg/kg, 1 μg/kg, 1.5 μg/kg, 2.0 μg/kg, 2.5 μg/kg, 3.0 μg/kg, 3.5 μg/kg, 4.0 μg/kg, 4.5 μg/kg, 5.0 μg/kg, 10 μg/kg, 15 μg/kg, 20 μg/kg, 25 μg/kg, 30 μg/kg, 35 μg/kg, 40 μg/kg, 45 μg/kg, 50 μg/kg, 55 μg/kg, 60 μg/kg, 65 μg/kg, 70 μg/kg, 75 μg/kg, 80 μg/kg, 85 μg/kg, 90 μg/kg, 95 μg/kg, 100 μg/kg, 150 μg/kg, 200 μg/kg, 250 μg/kg, 300 μg/kg, 350 μg/kg, 400 μg/kg, 450 μg/kg, 500 μg/kg, 550 μg/kg, 600 μg/kg, 650 μg/kg, 700 μg/kg, 750 μg/kg, 800 μg/kg, 850 μg/kg, 900 μg/kg, 1 mg/kg, 1.5 mg/kg, 2.0 mg/kg, 2.5 mg/kg, 3 mg/kg, 3.5 mg/kg, 4.0 mg/kg, 4.5 mg/kg, 5 mg/kg, 5.5 mg/kg, 6 mg/kg, 6.5 mg/kg, 7 mg/kg, 7.5 mg/kg, 8 mg/kg, 8.5 mg/kg, 9 mg/kg, 9.5 mg/kg, 10 mg/kg 10.5 mg/kg, 11 mg/kg, 11.5 mg/kg, 12 mg/kg, 12.5 mg/kg, 13 mg/kg, 13.5 mg/kg, 14 mg/kg, 14.5 mg/kg, 15 mg/kg, 16 mg/kg, 17 mg/kg, 18 mg/kg, 19 mg/kg, 20 mg/kg, 21 mg/kg, 22 mg/kg, 23 mg/kg, 24 mg/kg, 25 mg/kg, 30 mg/kg, 35 mg/kg, 40 mg/kg, 45 mg/kg, 50 mg/kg, 55 mg/kg, 60 mg/kg, 65 mg/kg, 70 mg/kg, 75 mg/kg, 80 mg/kg, 85 mg/kg, 90 mg/kg, 95 mg/kg, 100 mg/kg, 105 mg/kg, 110 mg/kg, 115 mg/kg, 120 mg/kg, 125 mg/kg, 130 mg/kg, 135 mg/kg, 140 mg/kg, 145 mg/kg, 150 mg/kg, 155 mg/kg, 160 mg/kg, 165 mg/kg, 170 mg/kg, 175 mg/kg, 180 mg/kg, 185 mg/kg, 190 mg/kg, 195 mg/kg, 200 mg/kg, and any fraction or integer in between any two of the preceding amounts.

In some embodiments, a NOS inhibitor composition comprising 1400W and/or a salt thereof, such as, for example, 1400W dihydrochloride, may be administered as a suitable dosage form, as described herein, to treat, ameliorate, and/or reduce the severity of ovarian cancer or one or more symptoms thereof in an ovarian cancer subject. In certain embodiments, an nNOS inhibitor composition comprising 1400W and/or a salt thereof, such as, for example, 1400W dihydrochloride, may be administered to an ovarian cancer subject to treat, ameliorate, and/or reduce the severity of ovarian cancer or one or more symptoms thereof in the ovarian cancer subject: (i) via a route selected from the group consisting of buccally, inhalation, intraarterially, intraarticularly, intracardially, intracerebroventricularly, intradermally, intramedullary, intramuscularly, intraperitoneally, intrathecally, intravenously, intraventricularly, nasally, ophthalmically, orally, rectally, sublabially, sublingually, subcutaneously, topically, and transdermally; and/or (ii) with a frequency selected from the group consisting of four times daily, three times daily, twice daily, once daily, once every two days, once every three days, once every four days, once every five days, once every six days, once a week, once every eight days, once every nine days, once every ten days, once every two weeks, once every three weeks, and once a month; and/or (iii) at a dosage (based on subject weight) selected from the group consisting of 0.1 μg/kg, 0.5 μg/kg, 1 μg/kg, 1.5 μg/kg, 2.0 μg/kg, 2.5 μg/kg, 3.0 μg/kg, 3.5 μg/kg, 4.0 μg/kg, 4.5 μg/kg, 5.0 μg/kg, 10 μg/kg, 15 μg/kg, 20 μg/kg, 25 μg/kg, 30 μg/kg, 35 μg/kg, 40 μg/kg, 45 μg/kg, 50 μg/kg, 55 μg/kg, 60 μg/kg, 65 μg/kg, 70 μg/kg, 75 μg/kg, 80 μg/kg, 85 μg/kg, 90 μg/kg, 95 μg/kg, 100 μg/kg, 150 μg/kg, 200 μg/kg, 250 μg/kg, 300 μg/kg, 350 μg/kg, 400 μg/kg, 450 μg/kg, 500 μg/kg, 550 μg/kg, 600 μg/kg, 650 μg/kg, 700 μg/kg, 750 μg/kg, 800 μg/kg, 850 μg/kg, 900 μg/kg, 1 mg/kg, 1.5 mg/kg, 2.0 mg/kg, 2.5 mg/kg, 3 mg/kg, 3.5 mg/kg, 4.0 mg/kg, 4.5 mg/kg, 5 mg/kg, 5.5 mg/kg, 6 mg/kg, 6.5 mg/kg, 7 mg/kg, 7.5 mg/kg, 8 mg/kg, 8.5 mg/kg, 9 mg/kg, 9.5 mg/kg, 10 mg/kg 10.5 mg/kg, 11 mg/kg, 11.5 mg/kg, 12 mg/kg, 12.5 mg/kg, 13 mg/kg, 13.5 mg/kg, 14 mg/kg, 14.5 mg/kg, 15 mg/kg, 16 mg/kg, 17 mg/kg, 18 mg/kg, 19 mg/kg, 20 mg/kg, 21 mg/kg, 22 mg/kg, 23 mg/kg, 24 mg/kg, 25 mg/kg, 30 mg/kg, 35 mg/kg, 40 mg/kg, 45 mg/kg, 50 mg/kg, 55 mg/kg, 60 mg/kg, 65 mg/kg, 70 mg/kg, 75 mg/kg, 80 mg/kg, 85 mg/kg, 90 mg/kg, 95 mg/kg, 100 mg/kg, 105 mg/kg, 110 mg/kg, 115 mg/kg, 120 mg/kg, 125 mg/kg, 130 mg/kg, 135 mg/kg, 140 mg/kg, 145 mg/kg, 150 mg/kg, 155 mg/kg, 160 mg/kg, 165 mg/kg, 170 mg/kg, 175 mg/kg, 180 mg/kg, 185 mg/kg, 190 mg/kg, 195 mg/kg, 200 mg/kg, and any fraction or integer in between any two of the preceding amounts.

In certain embodiments, a NOS inhibitor composition comprising OKN-007 and/or a salt thereof, such as, for example, OKN-007 sodium, may be administered as a suitable dosage form, as described herein, to treat, ameliorate, and/or reduce the severity of ovarian cancer or one or more symptoms thereof in an ovarian cancer subject. In some embodiments, an nNOS inhibitor composition comprising OKN-007 and/or a salt thereof, such as, for example, OKN-007 sodium, may be administered to an ovarian cancer subject to treat, ameliorate, and/or reduce the severity of ovarian cancer or one or more symptoms thereof in the ovarian cancer subject: (i) via a route selected from the group consisting of buccally, inhalation, intraarterially, intraarticularly, intracardially, intracerebroventricularly, intradermally, intramedullary, intramuscularly, intraperitoneally, intrathecally, intravenously, intraventricularly, nasally, ophthalmically, orally, rectally, sublabially, sublingually, subcutaneously, topically, and transdermally; and/or (ii) with a frequency selected from the group consisting of four times daily, three times daily, twice daily, once daily, once every two days, once every three days, once every four days, once every five days, once every six days, once a week, once every eight days, once every nine days, once every ten days, once every two weeks, once every three weeks, and once a month; and/or (iii) at a dosage (based on subject weight) selected from the group consisting of 0.1 μg/kg, 0.5 μg/kg, 1 μg/kg, 1.5 μg/kg, 2.0 μg/kg, 2.5 μg/kg, 3.0 μg/kg, 3.5 μg/kg, 4.0 μg/kg, 4.5 μg/kg, 5.0 μg/kg, 10 μg/kg, 15 μg/kg, 20 μg/kg, 25 μg/kg, 30 μg/kg, 35 μg/kg, 40 μg/kg, 45 μg/kg, 50 μg/kg, 55 μg/kg, 60 μg/kg, 65 μg/kg, 70 μg/kg, 75 μg/kg, 80 μg/kg, 85 μg/kg, 90 μg/kg, 95 μg/kg, 100 μg/kg, 150 μg/kg, 200 μg/kg, 250 μg/kg, 300 μg/kg, 350 μg/kg, 400 μg/kg, 450 μg/kg, 500 μg/kg, 550 μg/kg, 600 μg/kg, 650 μg/kg, 700 μg/kg, 750 μg/kg, 800 μg/kg, 850 μg/kg, 900 μg/kg, 1 mg/kg, 1.5 mg/kg, 2.0 mg/kg, 2.5 mg/kg, 3 mg/kg, 3.5 mg/kg, 4.0 mg/kg, 4.5 mg/kg, 5 mg/kg, 5.5 mg/kg, 6 mg/kg, 6.5 mg/kg, 7 mg/kg, 7.5 mg/kg, 8 mg/kg, 8.5 mg/kg, 9 mg/kg, 9.5 mg/kg, 10 mg/kg 10.5 mg/kg, 11 mg/kg, 11.5 mg/kg, 12 mg/kg, 12.5 mg/kg, 13 mg/kg, 13.5 mg/kg, 14 mg/kg, 14.5 mg/kg, 15 mg/kg, 16 mg/kg, 17 mg/kg, 18 mg/kg, 19 mg/kg, 20 mg/kg, 21 mg/kg, 22 mg/kg, 23 mg/kg, 24 mg/kg, 25 mg/kg, 30 mg/kg, 35 mg/kg, 40 mg/kg, 45 mg/kg, 50 mg/kg, 55 mg/kg, 60 mg/kg, 65 mg/kg, 70 mg/kg, 75 mg/kg, 80 mg/kg, 85 mg/kg, 90 mg/kg, 95 mg/kg, 100 mg/kg, 105 mg/kg, 110 mg/kg, 115 mg/kg, 120 mg/kg, 125 mg/kg, 130 mg/kg, 135 mg/kg, 140 mg/kg, 145 mg/kg, 150 mg/kg, 155 mg/kg, 160 mg/kg, 165 mg/kg, 170 mg/kg, 175 mg/kg, 180 mg/kg, 185 mg/kg, 190 mg/kg, 195 mg/kg, 200 mg/kg, and any fraction or integer in between any two of the preceding amounts.

In some embodiments, a NOS inhibitor composition comprising BYK-191023 and/or a salt thereof, such as, for example, BYK-191023 dihydrochloride, may be administered as a suitable dosage form, as described herein, to treat, ameliorate, and/or reduce the severity of ovarian cancer or one or more symptoms thereof in an ovarian cancer subject. In certain embodiments, an nNOS inhibitor composition comprising BYK-191023 and/or a salt thereof, such as, for example, BYK-191023 dihydrochloride, may be administered to an ovarian cancer subject to treat, ameliorate, and/or reduce the severity of ovarian cancer or one or more symptoms thereof in the ovarian cancer subject: (i) via a route selected from the group consisting of buccally, inhalation, intraarterially, intraarticularly, intracardially, intracerebroventricularly, intradermally, intramedullary, intramuscularly, intraperitoneally, intrathecally, intravenously, intraventricularly, nasally, ophthalmically, orally, rectally, sublabially, sublingually, subcutaneously, topically, and transdermally; and/or (ii) with a frequency selected from the group consisting of four times daily, three times daily, twice daily, once daily, once every two days, once every three days, once every four days, once every five days, once every six days, once a week, once every eight days, once every nine days, once every ten days, once every two weeks, once every three weeks, and once a month; and/or (iii) at a dosage (based on subject weight) selected from the group consisting of 0.1 μg/kg, 0.5 μg/kg, 1 μg/kg, 1.5 μg/kg, 2.0 μg/kg, 2.5 μg/kg, 3.0 μg/kg, 3.5 μg/kg, 4.0 μg/kg, 4.5 μg/kg, 5.0 μg/kg, 10 μg/kg, 15 μg/kg, 20 μg/kg, 25 μg/kg, 30 μg/kg, 35 μg/kg, 40 μg/kg, 45 μg/kg, 50 μg/kg, 55 μg/kg, 60 μg/kg, 65 μg/kg, 70 μg/kg, 75 μg/kg, 80 μg/kg, 85 μg/kg, 90 μg/kg, 95 μg/kg, 100 μg/kg, 150 μg/kg, 200 μg/kg, 250 μg/kg, 300 μg/kg, 350 μg/kg, 400 μg/kg, 450 μg/kg, 500 μg/kg, 550 μg/kg, 600 μg/kg, 650 μg/kg, 700 μg/kg, 750 μg/kg, 800 μg/kg, 850 μg/kg, 900 μg/kg, 1 mg/kg, 1.5 mg/kg, 2.0 mg/kg, 2.5 mg/kg, 3 mg/kg, 3.5 mg/kg, 4.0 mg/kg, 4.5 mg/kg, 5 mg/kg, 5.5 mg/kg, 6 mg/kg, 6.5 mg/kg, 7 mg/kg, 7.5 mg/kg, 8 mg/kg, 8.5 mg/kg, 9 mg/kg, 9.5 mg/kg, 10 mg/kg 10.5 mg/kg, 11 mg/kg, 11.5 mg/kg, 12 mg/kg, 12.5 mg/kg, 13 mg/kg, 13.5 mg/kg, 14 mg/kg, 14.5 mg/kg, 15 mg/kg, 16 mg/kg, 17 mg/kg, 18 mg/kg, 19 mg/kg, 20 mg/kg, 21 mg/kg, 22 mg/kg, 23 mg/kg, 24 mg/kg, 25 mg/kg, 30 mg/kg, 35 mg/kg, 40 mg/kg, 45 mg/kg, 50 mg/kg, 55 mg/kg, 60 mg/kg, 65 mg/kg, 70 mg/kg, 75 mg/kg, 80 mg/kg, 85 mg/kg, 90 mg/kg, 95 mg/kg, 100 mg/kg, 105 mg/kg, 110 mg/kg, 115 mg/kg, 120 mg/kg, 125 mg/kg, 130 mg/kg, 135 mg/kg, 140 mg/kg, 145 mg/kg, 150 mg/kg, 155 mg/kg, 160 mg/kg, 165 mg/kg, 170 mg/kg, 175 mg/kg, 180 mg/kg, 185 mg/kg, 190 mg/kg, 195 mg/kg, 200 mg/kg, and any fraction or integer in between any two of the preceding amounts.

In certain embodiments, a NOS inhibitor composition comprising CM544 and/or a salt thereof, such as, for example, CM544 dihydrochloride, may be administered as a suitable dosage form, as described herein, to treat, ameliorate, and/or reduce the severity of ovarian cancer or one or more symptoms thereof in an ovarian cancer subject. In some embodiments, an nNOS inhibitor composition comprising CM544 and/or a salt thereof, such as, for example, CM544 dihydrochloride, may be administered to an ovarian cancer subject to treat, ameliorate, and/or reduce the severity of ovarian cancer or one or more symptoms thereof in the ovarian cancer subject: (i) via a route selected from the group consisting of buccally, inhalation, intraarterially, intraarticularly, intracardially, intracerebroventricularly, intradermally, intramedullary, intramuscularly, intraperitoneally, intrathecally, intravenously, intraventricularly, nasally, ophthalmically, orally, rectally, sublabially, sublingually, subcutaneously, topically, and transdermally; and/or (ii) with a frequency selected from the group consisting of four times daily, three times daily, twice daily, once daily, once every two days, once every three days, once every four days, once every five days, once every six days, once a week, once every eight days, once every nine days, once every ten days, once every two weeks, once every three weeks, and once a month; and/or (iii) at a dosage (based on subject weight) selected from the group consisting of 0.1 μg/kg, 0.5 μg/kg, 1 μg/kg, 1.5 μg/kg, 2.0 μg/kg, 2.5 μg/kg, 3.0 μg/kg, 3.5 μg/kg, 4.0 μg/kg, 4.5 μg/kg, 5.0 μg/kg, 10 μg/kg, 15 μg/kg, 20 μg/kg, 25 μg/kg, 30 μg/kg, 35 μg/kg, 40 μg/kg, 45 μg/kg, 50 μg/kg, 55 μg/kg, 60 μg/kg, 65 μg/kg, 70 μg/kg, 75 μg/kg, 80 μg/kg, 85 μg/kg, 90 μg/kg, 95 μg/kg, 100 μg/kg, 150 μg/kg, 200 μg/kg, 250 μg/kg, 300 μg/kg, 350 μg/kg, 400 μg/kg, 450 μg/kg, 500 μg/kg, 550 μg/kg, 600 μg/kg, 650 μg/kg, 700 μg/kg, 750 μg/kg, 800 μg/kg, 850 μg/kg, 900 μg/kg, 1 mg/kg, 1.5 mg/kg, 2.0 mg/kg, 2.5 mg/kg, 3 mg/kg, 3.5 mg/kg, 4.0 mg/kg, 4.5 mg/kg, 5 mg/kg, 5.5 mg/kg, 6 mg/kg, 6.5 mg/kg, 7 mg/kg, 7.5 mg/kg, 8 mg/kg, 8.5 mg/kg, 9 mg/kg, 9.5 mg/kg, 10 mg/kg 10.5 mg/kg, 11 mg/kg, 11.5 mg/kg, 12 mg/kg, 12.5 mg/kg, 13 mg/kg, 13.5 mg/kg, 14 mg/kg, 14.5 mg/kg, 15 mg/kg, 16 mg/kg, 17 mg/kg, 18 mg/kg, 19 mg/kg, 20 mg/kg, 21 mg/kg, 22 mg/kg, 23 mg/kg, 24 mg/kg, 25 mg/kg, 30 mg/kg, 35 mg/kg, 40 mg/kg, 45 mg/kg, 50 mg/kg, 55 mg/kg, 60 mg/kg, 65 mg/kg, 70 mg/kg, 75 mg/kg, 80 mg/kg, 85 mg/kg, 90 mg/kg, 95 mg/kg, 100 mg/kg, 105 mg/kg, 110 mg/kg, 115 mg/kg, 120 mg/kg, 125 mg/kg, 130 mg/kg, 135 mg/kg, 140 mg/kg, 145 mg/kg, 150 mg/kg, 155 mg/kg, 160 mg/kg, 165 mg/kg, 170 mg/kg, 175 mg/kg, 180 mg/kg, 185 mg/kg, 190 mg/kg, 195 mg/kg, 200 mg/kg, and any fraction or integer in between any two of the preceding amounts.

In certain embodiments, an nNOS inhibitor composition comprising one or more dsRNAs each comprising at least 30 base pairs, may be administered as a suitable dosage form, as described herein, to treat, ameliorate, and/or reduce the severity of ovarian cancer or one or more symptoms thereof in an ovarian cancer subject. In some embodiments, an nNOS inhibitor composition comprising one or more dsRNAs, each comprising at least 30 base pairs, may be administered to an ovarian cancer subject to treat, ameliorate, and/or reduce the severity of ovarian cancer or one or more symptoms thereof in the ovarian cancer subject: (i) via a route selected from the group consisting of buccally, inhalation, intraarterially, intraarticularly, intracardially, intracerebroventricularly, intradermally, intramedullary, intramuscularly, intraperitoneally, intrathecally, intravenously, intraventricularly, nasally, ophthalmically, orally, rectally, sublabially, sublingually, subcutaneously, topically, and transdermally; and/or (ii) with a frequency selected from the group consisting of four times daily, three times daily, twice daily, once daily, once every two days, once every three days, once every four days, once every five days, once every six days, once a week, once every eight days, once every nine days, once every ten days, once every two weeks, once every three weeks, and once a month; and/or (iii) at a dosage (based on subject weight) selected from the group consisting of 0.1 μg/kg, 0.5 μg/kg, 1 μg/kg, 1.5 μg/kg, 2.0 μg/kg, 2.5 μg/kg, 3.0 μg/kg, 3.5 μg/kg, 4.0 μg/kg, 4.5 μg/kg, 5.0 μg/kg, 10 μg/kg, 15 μg/kg, 20 μg/kg, 25 μg/kg, 30 μg/kg, 35 μg/kg, 40 μg/kg, 45 μg/kg, 50 μg/kg, 55 μg/kg, 60 μg/kg, 65 μg/kg, 70 μg/kg, 75 μg/kg, 80 μg/kg, 85 μg/kg, 90 μg/kg, 95 μg/kg, 100 μg/kg, 150 μg/kg, 200 μg/kg, 250 μg/kg, 300 μg/kg, 350 μg/kg, 400 μg/kg, 450 μg/kg, 500 μg/kg, 550 μg/kg, 600 μg/kg, 650 μg/kg, 700 μg/kg, 750 μg/kg, 800 μg/kg, 850 μg/kg, 900 μg/kg, 1 mg/kg, 1.5 mg/kg, 2.0 mg/kg, 2.5 mg/kg, 3 mg/kg, 3.5 mg/kg, 4.0 mg/kg, 4.5 mg/kg, 5 mg/kg, 5.5 mg/kg, 6 mg/kg, 6.5 mg/kg, 7 mg/kg, 7.5 mg/kg, 8 mg/kg, 8.5 mg/kg, 9 mg/kg, 9.5 mg/kg, 10 mg/kg 10.5 mg/kg, 11 mg/kg, 11.5 mg/kg, 12 mg/kg, 12.5 mg/kg, 13 mg/kg, 13.5 mg/kg, 14 mg/kg, 14.5 mg/kg, 15 mg/kg, 16 mg/kg, 17 mg/kg, 18 mg/kg, 19 mg/kg, 20 mg/kg, 21 mg/kg, 22 mg/kg, 23 mg/kg, 24 mg/kg, 25 mg/kg, 30 mg/kg, 35 mg/kg, 40 mg/kg, 45 mg/kg, 50 mg/kg, 55 mg/kg, 60 mg/kg, 65 mg/kg, 70 mg/kg, 75 mg/kg, 80 mg/kg, 85 mg/kg, 90 mg/kg, 95 mg/kg, 100 mg/kg, 105 mg/kg, 110 mg/kg, 115 mg/kg, 120 mg/kg, 125 mg/kg, 130 mg/kg, 135 mg/kg, 140 mg/kg, 145 mg/kg, 150 mg/kg, 155 mg/kg, 160 mg/kg, 165 mg/kg, 170 mg/kg, 175 mg/kg, 180 mg/kg, 185 mg/kg, 190 mg/kg, 195 mg/kg, 200 mg/kg, and any fraction or integer in between any two of the preceding amounts.

In some embodiments, an nNOS inhibitor composition comprising one or more siRNAs, may be administered as a suitable dosage form, as described herein, to treat, ameliorate, and/or reduce the severity of ovarian cancer or one or more symptoms thereof in an ovarian cancer subject. In certain embodiments, an nNOS inhibitor composition comprising one or more siRNAs selected from the group consisting of SEQ ID NO. 4, SEQ ID NO. 5, SEQ ID NO. 6, SEQ ID NO. 7, SEQ ID NO. 8, and any combination thereof, may be administered as a suitable dosage form, as described herein, to treat, ameliorate, and/or reduce the severity of ovarian cancer or one or more symptoms thereof in an ovarian cancer subject. In some embodiments, an nNOS inhibitor composition comprising one or more siRNAs selected from the group consisting of SEQ ID NO. 4, SEQ ID NO. 5, SEQ ID NO. 6, SEQ ID NO. 7, SEQ ID NO. 8, and any combination thereof, may be administered to an ovarian cancer subject to treat, ameliorate, and/or reduce the severity of ovarian cancer or one or more symptoms thereof in the ovarian cancer subject: (i) via a route selected from the group consisting of buccally, inhalation, intraarterially, intraarticularly, intracardially, intracerebroventricularly, intradermally, intramedullary, intramuscularly, intraperitoneally, intrathecally, intravenously, intraventricularly, nasally, ophthalmically, orally, rectally, sublabially, sublingually, subcutaneously, topically, and transdermally; and/or (ii) with a frequency selected from the group consisting of four times daily, three times daily, twice daily, once daily, once every two days, once every three days, once every four days, once every five days, once every six days, once a week, once every eight days, once every nine days, once every ten days, once every two weeks, once every three weeks, and once a month; and/or (iii) at a dosage (based on subject weight) selected from the group consisting of 0.1 μg/kg, 0.5 μg/kg, 1 μg/kg, 1.5 μg/kg, 2.0 μg/kg, 2.5 μg/kg, 3.0 μg/kg, 3.5 μg/kg, 4.0 μg/kg, 4.5 μg/kg, 5.0 μg/kg, 10 μg/kg, 15 μg/kg, 20 μg/kg, 25 μg/kg, 30 μg/kg, 35 μg/kg, 40 μg/kg, 45 μg/kg, 50 μg/kg, 55 μg/kg, 60 μg/kg, 65 μg/kg, 70 μg/kg, 75 μg/kg, 80 μg/kg, 85 μg/kg, 90 μg/kg, 95 μg/kg, 100 μg/kg, 150 μg/kg, 200 μg/kg, 250 μg/kg, 300 μg/kg, 350 μg/kg, 400 μg/kg, 450 μg/kg, 500 μg/kg, 550 μg/kg, 600 μg/kg, 650 μg/kg, 700 μg/kg, 750 μg/kg, 800 μg/kg, 850 μg/kg, 900 μg/kg, 1 mg/kg, 1.5 mg/kg, 2.0 mg/kg, 2.5 mg/kg, 3 mg/kg, 3.5 mg/kg, 4.0 mg/kg, 4.5 mg/kg, 5 mg/kg, 5.5 mg/kg, 6 mg/kg, 6.5 mg/kg, 7 mg/kg, 7.5 mg/kg, 8 mg/kg, 8.5 mg/kg, 9 mg/kg, 9.5 mg/kg, 10 mg/kg 10.5 mg/kg, 11 mg/kg, 11.5 mg/kg, 12 mg/kg, 12.5 mg/kg, 13 mg/kg, 13.5 mg/kg, 14 mg/kg, 14.5 mg/kg, 15 mg/kg, 16 mg/kg, 17 mg/kg, 18 mg/kg, 19 mg/kg, 20 mg/kg, 21 mg/kg, 22 mg/kg, 23 mg/kg, 24 mg/kg, 25 mg/kg, 30 mg/kg, 35 mg/kg, 40 mg/kg, 45 mg/kg, 50 mg/kg, 55 mg/kg, 60 mg/kg, 65 mg/kg, 70 mg/kg, 75 mg/kg, 80 mg/kg, 85 mg/kg, 90 mg/kg, 95 mg/kg, 100 mg/kg, 105 mg/kg, 110 mg/kg, 115 mg/kg, 120 mg/kg, 125 mg/kg, 130 mg/kg, 135 mg/kg, 140 mg/kg, 145 mg/kg, 150 mg/kg, 155 mg/kg, 160 mg/kg, 165 mg/kg, 170 mg/kg, 175 mg/kg, 180 mg/kg, 185 mg/kg, 190 mg/kg, 195 mg/kg, 200 mg/kg, and any fraction or integer in between any two of the preceding amounts.

In some embodiments, an nNOS inhibitor composition comprising one or more shRNAs, may be administered as a suitable dosage form, as described herein, to treat, ameliorate, and/or reduce the severity of ovarian cancer or one or more symptoms thereof in an ovarian cancer subject. In certain embodiments, an nNOS inhibitor composition comprising one or more shRNAs selected from the group consisting of SEQ ID NO. 9, SEQ ID NO. 10, SEQ ID NO. 11, SEQ ID NO. 12, SEQ ID NO. 13, SEQ ID NO. 14, and any combination thereof, may be administered as a suitable dosage form, as described herein, to treat, ameliorate, and/or reduce the severity of ovarian cancer or one or more symptoms thereof in an ovarian cancer subject. In some embodiments, an nNOS inhibitor composition comprising one or more shRNAs selected from the group consisting of SEQ ID NO. 9, SEQ ID NO. 10, SEQ ID NO. 11, SEQ ID NO. 12, SEQ ID NO. 13, SEQ ID NO. 14, and any combination thereof, may be administered to an ovarian cancer subject to treat, ameliorate, and/or reduce the severity of ovarian cancer or one or more symptoms thereof in the ovarian cancer subject: (i) via a route selected from the group consisting of buccally, inhalation, intraarterially, intraarticularly, intracardially, intracerebroventricularly, intradermally, intramedullary, intramuscularly, intraperitoneally, intrathecally, intravenously, intraventricularly, nasally, ophthalmically, orally, rectally, sublabially, sublingually, subcutaneously, topically, and transdermally; and/or (ii) with a frequency selected from the group consisting of four times daily, three times daily, twice daily, once daily, once every two days, once every three days, once every four days, once every five days, once every six days, once a week, once every eight days, once every nine days, once every ten days, once every two weeks, once every three weeks, and once a month; and/or (iii) at a dosage (based on subject weight) selected from the group consisting of 0.1 μg/kg, 0.5 μg/kg, 1 μg/kg, 1.5 μg/kg, 2.0 μg/kg, 2.5 μg/kg, 3.0 μg/kg, 3.5 μg/kg, 4.0 μg/kg, 4.5 μg/kg, 5.0 μg/kg, 10 μg/kg, 15 μg/kg, 20 μg/kg, 25 μg/kg, 30 μg/kg, 35 μg/kg, 40 μg/kg, 45 μg/kg, 50 μg/kg, 55 μg/kg, 60 μg/kg, 65 μg/kg, 70 μg/kg, 75 μg/kg, 80 μg/kg, 85 μg/kg, 90 μg/kg, 95 μg/kg, 100 μg/kg, 150 μg/kg, 200 μg/kg, 250 μg/kg, 300 μg/kg, 350 μg/kg, 400 μg/kg, 450 μg/kg, 500 μg/kg, 550 μg/kg, 600 μg/kg, 650 μg/kg, 700 μg/kg, 750 μg/kg, 800 μg/kg, 850 μg/kg, 900 μg/kg, 1 mg/kg, 1.5 mg/kg, 2.0 mg/kg, 2.5 mg/kg, 3 mg/kg, 3.5 mg/kg, 4.0 mg/kg, 4.5 mg/kg, 5 mg/kg, 5.5 mg/kg, 6 mg/kg, 6.5 mg/kg, 7 mg/kg, 7.5 mg/kg, 8 mg/kg, 8.5 mg/kg, 9 mg/kg, 9.5 mg/kg, 10 mg/kg 10.5 mg/kg, 11 mg/kg, 11.5 mg/kg, 12 mg/kg, 12.5 mg/kg, 13 mg/kg, 13.5 mg/kg, 14 mg/kg, 14.5 mg/kg, 15 mg/kg, 16 mg/kg, 17 mg/kg, 18 mg/kg, 19 mg/kg, 20 mg/kg, 21 mg/kg, 22 mg/kg, 23 mg/kg, 24 mg/kg, 25 mg/kg, 30 mg/kg, 35 mg/kg, 40 mg/kg, 45 mg/kg, 50 mg/kg, 55 mg/kg, 60 mg/kg, 65 mg/kg, 70 mg/kg, 75 mg/kg, 80 mg/kg, 85 mg/kg, 90 mg/kg, 95 mg/kg, 100 mg/kg, 105 mg/kg, 110 mg/kg, 115 mg/kg, 120 mg/kg, 125 mg/kg, 130 mg/kg, 135 mg/kg, 140 mg/kg, 145 mg/kg, 150 mg/kg, 155 mg/kg, 160 mg/kg, 165 mg/kg, 170 mg/kg, 175 mg/kg, 180 mg/kg, 185 mg/kg, 190 mg/kg, 195 mg/kg, 200 mg/kg, and any fraction or integer in between any two of the preceding amounts.

In some embodiments, an nNOS inhibitor composition comprising one or more miRNAs, may be administered as a suitable dosage form, as described herein, to treat, ameliorate, and/or reduce the severity of ovarian cancer or one or more symptoms thereof in an ovarian cancer subject. In certain embodiments, an nNOS inhibitor composition comprising one or more miRNAs selected from the group consisting of miR-146a, hsa-miR-146a-5p, hsa-miR-1273f, hsa-miR-143-3p, hsa-miR-147a, hsa-miR-3622a-5p, hsa-miR-3911, hsa-miR-4269, hsa-miR-4708-5p, hsa-miR-4710, hsa-miR-4770, hsa-miR-4792, hsa-miR-574-5p, hsa-miR-6088, hsa-miR-644a, hsa-miR-6715b-5p, hsa-miR-6768-5p, hsa-miR-6867-5p, hsa-miR-7855-5p, hsa-miR-8485, and any combination thereof, may be administered as a suitable dosage form, as described herein, to treat, ameliorate, and/or reduce the severity of ovarian cancer or one or more symptoms thereof in an ovarian cancer subject. In some embodiments, an nNOS inhibitor composition comprising one or more miRNAs selected from the group consisting of miR-146a, hsa-miR-146a-5p, hsa-miR-1273f, hsa-miR-143-3p, hsa-miR-147a, hsa-miR-3622a-5p, hsa-miR-3911, hsa-miR-4269, hsa-miR-4708-5p, hsa-miR-4710, hsa-miR-4770, hsa-miR-4792, hsa-miR-574-5p, hsa-miR-6088, hsa-miR-644a, hsa-miR-6715b-5p, hsa-miR-6768-5p, hsa-miR-6867-5p, hsa-miR-7855-5p, hsa-miR-8485, and any combination thereof, may be administered to an ovarian cancer subject to treat, ameliorate, and/or reduce the severity of ovarian cancer or one or more symptoms thereof in the ovarian cancer subject: (i) via a route selected from the group consisting of buccally, inhalation, intraarterially, intraarticularly, intracardially, intracerebroventricularly, intradermally, intramedullary, intramuscularly, intraperitoneally, intrathecally, intravenously, intraventricularly, nasally, ophthalmically, orally, rectally, sublabially, sublingually, subcutaneously, topically, and transdermally; and/or (ii) with a frequency selected from the group consisting of four times daily, three times daily, twice daily, once daily, once every two days, once every three days, once every four days, once every five days, once every six days, once a week, once every eight days, once every nine days, once every ten days, once every two weeks, once every three weeks, and once a month; and/or (iii) at a dosage (based on subject weight) selected from the group consisting of 0.1 μg/kg, 0.5 μg/kg, 1 μg/kg, 1.5 μg/kg, 2.0 μg/kg, 2.5 μg/kg, 3.0 μg/kg, 3.5 μg/kg, 4.0 μg/kg, 4.5 μg/kg, 5.0 μg/kg, 10 μg/kg, 15 μg/kg, 20 μg/kg, 25 μg/kg, 30 μg/kg, 35 μg/kg, 40 μg/kg, 45 μg/kg, 50 μg/kg, 55 μg/kg, 60 μg/kg, 65 μg/kg, 70 μg/kg, 75 μg/kg, 80 μg/kg, 85 μg/kg, 90 μg/kg, 95 μg/kg, 100 μg/kg, 150 μg/kg, 200 μg/kg, 250 μg/kg, 300 μg/kg, 350 μg/kg, 400 μg/kg, 450 μg/kg, 500 μg/kg, 550 μg/kg, 600 μg/kg, 650 μg/kg, 700 μg/kg, 750 μg/kg, 800 μg/kg, 850 μg/kg, 900 μg/kg, 1 mg/kg, 1.5 mg/kg, 2.0 mg/kg, 2.5 mg/kg, 3 mg/kg, 3.5 mg/kg, 4.0 mg/kg, 4.5 mg/kg, 5 mg/kg, 5.5 mg/kg, 6 mg/kg, 6.5 mg/kg, 7 mg/kg, 7.5 mg/kg, 8 mg/kg, 8.5 mg/kg, 9 mg/kg, 9.5 mg/kg, 10 mg/kg 10.5 mg/kg, 11 mg/kg, 11.5 mg/kg, 12 mg/kg, 12.5 mg/kg, 13 mg/kg, 13.5 mg/kg, 14 mg/kg, 14.5 mg/kg, 15 mg/kg, 16 mg/kg, 17 mg/kg, 18 mg/kg, 19 mg/kg, 20 mg/kg, 21 mg/kg, 22 mg/kg, 23 mg/kg, 24 mg/kg, 25 mg/kg, 30 mg/kg, 35 mg/kg, 40 mg/kg, 45 mg/kg, 50 mg/kg, 55 mg/kg, 60 mg/kg, 65 mg/kg, 70 mg/kg, 75 mg/kg, 80 mg/kg, 85 mg/kg, 90 mg/kg, 95 mg/kg, 100 mg/kg, 105 mg/kg, 110 mg/kg, 115 mg/kg, 120 mg/kg, 125 mg/kg, 130 mg/kg, 135 mg/kg, 140 mg/kg, 145 mg/kg, 150 mg/kg, 155 mg/kg, 160 mg/kg, 165 mg/kg, 170 mg/kg, 175 mg/kg, 180 mg/kg, 185 mg/kg, 190 mg/kg, 195 mg/kg, 200 mg/kg, and any fraction or integer in between any two of the preceding amounts.

In some embodiments, an nNOS inhibitor composition comprising one or more CRISPR/Cas systems, may be administered as a suitable dosage form, as described herein, to treat, ameliorate, and/or reduce the severity of ovarian cancer or one or more symptoms thereof in an ovarian cancer subject. In certain embodiments, an nNOS inhibitor composition comprising one or more CRISPR/Cas systems comprising an endonuclease selected from the group consisting of Cas1, Cas2, Cas3, Cas4, Cas5, Cas6, Cas7, Cas8a, Cas8b, Cas8c, Cas9, Cas10, Cas10d, Cas12, Cas12a, Cas12b, Cas12c, Cas12d, Cas12e, Cas12f, Cas12g, Cas12h, Cas12i, Cas12k, C2c4, C2c8, C2c9, Cas13, Cas13a, Cas13b, Cas13c, Cas13d, Cas13x.1, Cse1, Cse2, Csy1, Csy2, Csy3, GSU0054, Csm2, Cmr5, Csx10, Csx11, Csf1, and Csn2, may be administered as a suitable dosage form, as described herein, to treat, ameliorate, and/or reduce the severity of ovarian cancer or one or more symptoms thereof in an ovarian cancer subject. In some embodiments, an nNOS inhibitor composition comprising one or more CRISPR/Cas systems comprising an endonuclease selected from the group consisting of Cas1, Cas2, Cas3, Cas4, Cas5, Cas6, Cas7, Cas8a, Cas8b, Cas8c, Cas9, Cas10, Cas10d, Cas12, Cas12a, Cas12b, Cas12c, Cas12d, Cas12e, Cas12f, Cas12g, Cas12h, Cas12i, Cas12k, C2c4, C2c8, C2c9, Cas13, Cas13a, Cas13b, Cas13c, Cas13d, Cas13x.1, Cse1, Cse2, Csy1, Csy2, Csy3, GSU0054, Csm2, Cmr5, Csx10, Csx11, Csf1, and Csn2, may be administered to an ovarian cancer subject to treat, ameliorate, and/or reduce the severity of ovarian cancer or one or more symptoms thereof in the ovarian cancer subject: (i) via a route selected from the group consisting of buccally, inhalation, intraarterially, intraarticularly, intracardially, intracerebroventricularly, intradermally, intramedullary, intramuscularly, intraperitoneally, intrathecally, intravenously, intraventricularly, nasally, ophthalmically, orally, rectally, sublabially, sublingually, subcutaneously, topically, and transdermally; and/or (ii) with a frequency selected from the group consisting of four times daily, three times daily, twice daily, once daily, once every two days, once every three days, once every four days, once every five days, once every six days, once a week, once every eight days, once every nine days, once every ten days, once every two weeks, once every three weeks, and once a month; and/or (iii) at a dosage (based on subject weight) selected from the group consisting of 0.1 μg/kg, 0.5 μg/kg, 1 μg/kg, 1.5 μg/kg, 2.0 μg/kg, 2.5 μg/kg, 3.0 μg/kg, 3.5 μg/kg, 4.0 μg/kg, 4.5 μg/kg, 5.0 μg/kg, 10 μg/kg, 15 μg/kg, 20 μg/kg, 25 μg/kg, 30 μg/kg, 35 μg/kg, 40 μg/kg, 45 μg/kg, 50 μg/kg, 55 μg/kg, 60 μg/kg, 65 μg/kg, 70 μg/kg, 75 μg/kg, 80 μg/kg, 85 μg/kg, 90 μg/kg, 95 μg/kg, 100 μg/kg, 150 μg/kg, 200 μg/kg, 250 μg/kg, 300 μg/kg, 350 μg/kg, 400 μg/kg, 450 μg/kg, 500 μg/kg, 550 μg/kg, 600 μg/kg, 650 μg/kg, 700 μg/kg, 750 μg/kg, 800 μg/kg, 850 μg/kg, 900 μg/kg, 1 mg/kg, 1.5 mg/kg, 2.0 mg/kg, 2.5 mg/kg, 3 mg/kg, 3.5 mg/kg, 4.0 mg/kg, 4.5 mg/kg, 5 mg/kg, 5.5 mg/kg, 6 mg/kg, 6.5 mg/kg, 7 mg/kg, 7.5 mg/kg, 8 mg/kg, 8.5 mg/kg, 9 mg/kg, 9.5 mg/kg, 10 mg/kg 10.5 mg/kg, 11 mg/kg, 11.5 mg/kg, 12 mg/kg, 12.5 mg/kg, 13 mg/kg, 13.5 mg/kg, 14 mg/kg, 14.5 mg/kg, 15 mg/kg, 16 mg/kg, 17 mg/kg, 18 mg/kg, 19 mg/kg, 20 mg/kg, 21 mg/kg, 22 mg/kg, 23 mg/kg, 24 mg/kg, 25 mg/kg, 30 mg/kg, 35 mg/kg, 40 mg/kg, 45 mg/kg, 50 mg/kg, 55 mg/kg, 60 mg/kg, 65 mg/kg, 70 mg/kg, 75 mg/kg, 80 mg/kg, 85 mg/kg, 90 mg/kg, 95 mg/kg, 100 mg/kg, 105 mg/kg, 110 mg/kg, 115 mg/kg, 120 mg/kg, 125 mg/kg, 130 mg/kg, 135 mg/kg, 140 mg/kg, 145 mg/kg, 150 mg/kg, 155 mg/kg, 160 mg/kg, 165 mg/kg, 170 mg/kg, 175 mg/kg, 180 mg/kg, 185 mg/kg, 190 mg/kg, 195 mg/kg, 200 mg/kg, and any fraction or integer in between any two of the preceding amounts.

In some embodiments, an nNOS inhibitor composition comprising one or more antibodies, may be administered as a suitable dosage form, as described herein, to treat, ameliorate, and/or reduce the severity of ovarian cancer or one or more symptoms thereof in an ovarian cancer subject. In certain embodiments, an nNOS inhibitor composition comprising one or more antibodies selected from the group consisting of 37-2800 (from ThermoFisher), EP1855Y (from abeam), EPR 24351-6 (from abeam), and any combination thereof, may be administered as a suitable dosage form, as described herein, to treat, ameliorate, and/or reduce the severity of ovarian cancer or one or more symptoms thereof in an ovarian cancer subject. In some embodiments, an nNOS inhibitor composition comprising one or more antibodies selected from the group consisting of 37-2800 (from ThermoFisher), EP1855Y (from abeam), EPR 24351-6 (from abeam), and any combination thereof, may be administered to an ovarian cancer subject to treat, ameliorate, and/or reduce the severity of ovarian cancer or one or more symptoms thereof in the ovarian cancer subject: (i) via a route selected from the group consisting of buccally, inhalation, intraarterially, intraarticularly, intracardially, intracerebroventricularly, intradermally, intramedullary, intramuscularly, intraperitoneally, intrathecally, intravenously, intraventricularly, nasally, ophthalmically, orally, rectally, sublabially, sublingually, subcutaneously, topically, and transdermally; and/or (ii) with a frequency selected from the group consisting of four times daily, three times daily, twice daily, once daily, once every two days, once every three days, once every four days, once every five days, once every six days, once a week, once every eight days, once every nine days, once every ten days, once every two weeks, once every three weeks, and once a month; and/or (iii) at a dosage (based on subject weight) selected from the group consisting of 0.1 μg/kg, 0.5 μg/kg, 1 μg/kg, 1.5 μg/kg, 2.0 μg/kg, 2.5 μg/kg, 3.0 μg/kg, 3.5 μg/kg, 4.0 μg/kg, 4.5 μg/kg, 5.0 μg/kg, 10 μg/kg, 15 μg/kg, 20 μg/kg, 25 μg/kg, 30 μg/kg, 35 μg/kg, 40 μg/kg, 45 μg/kg, 50 μg/kg, 55 μg/kg, 60 μg/kg, 65 μg/kg, 70 μg/kg, 75 μg/kg, 80 μg/kg, 85 μg/kg, 90 μg/kg, 95 μg/kg, 100 μg/kg, 150 μg/kg, 200 μg/kg, 250 μg/kg, 300 μg/kg, 350 μg/kg, 400 μg/kg, 450 μg/kg, 500 μg/kg, 550 μg/kg, 600 μg/kg, 650 μg/kg, 700 μg/kg, 750 μg/kg, 800 μg/kg, 850 μg/kg, 900 μg/kg, 1 mg/kg, 1.5 mg/kg, 2.0 mg/kg, 2.5 mg/kg, 3 mg/kg, 3.5 mg/kg, 4.0 mg/kg, 4.5 mg/kg, 5 mg/kg, 5.5 mg/kg, 6 mg/kg, 6.5 mg/kg, 7 mg/kg, 7.5 mg/kg, 8 mg/kg, 8.5 mg/kg, 9 mg/kg, 9.5 mg/kg, 10 mg/kg 10.5 mg/kg, 11 mg/kg, 11.5 mg/kg, 12 mg/kg, 12.5 mg/kg, 13 mg/kg, 13.5 mg/kg, 14 mg/kg, 14.5 mg/kg, 15 mg/kg, 16 mg/kg, 17 mg/kg, 18 mg/kg, 19 mg/kg, 20 mg/kg, 21 mg/kg, 22 mg/kg, 23 mg/kg, 24 mg/kg, 25 mg/kg, 30 mg/kg, 35 mg/kg, 40 mg/kg, 45 mg/kg, 50 mg/kg, 55 mg/kg, 60 mg/kg, 65 mg/kg, 70 mg/kg, 75 mg/kg, 80 mg/kg, 85 mg/kg, 90 mg/kg, 95 mg/kg, 100 mg/kg, 105 mg/kg, 110 mg/kg, 115 mg/kg, 120 mg/kg, 125 mg/kg, 130 mg/kg, 135 mg/kg, 140 mg/kg, 145 mg/kg, 150 mg/kg, 155 mg/kg, 160 mg/kg, 165 mg/kg, 170 mg/kg, 175 mg/kg, 180 mg/kg, 185 mg/kg, 190 mg/kg, 195 mg/kg, 200 mg/kg, and any fraction or integer in between any two of the preceding amounts.

In some embodiments, an nNOS inhibitor composition comprising one or more amino acid-based compounds, may be administered as a suitable dosage form, as described herein, to treat, ameliorate, and/or reduce the severity of ovarian cancer or one or more symptoms thereof in an ovarian cancer subject. In certain embodiments, an nNOS inhibitor composition comprising one or more amino acid-compounds that bind an active site of nNOS selected from the group consisting of Phenylalanine 589, Tryptophan 592, Tyrosine 593, Glutamate 597, Aspartate 602, Arginine 601, Methionine 341, Histidine 342, and Tyrosine711, may be administered as a suitable dosage form, as described herein, to treat, ameliorate, and/or reduce the severity of ovarian cancer or one or more symptoms thereof in an ovarian cancer subject. In some embodiments, an nNOS inhibitor composition comprising one or more amino acid-compounds that bind an active site of nNOS selected from the group consisting of Phenylalanine 589, Tryptophan 592, Tyrosine 593, Glutamate 597, Aspartate 602, Arginine 601, Methionine 341, Histidine 342, and Tyrosine711, may be administered to an ovarian cancer subject to treat, ameliorate, and/or reduce the severity of ovarian cancer or one or more symptoms thereof in the ovarian cancer subject: (i) via a route selected from the group consisting of buccally, inhalation, intraarterially, intraarticularly, intracardially, intracerebroventricularly, intradermally, intramedullary, intramuscularly, intraperitoneally, intrathecally, intravenously, intraventricularly, nasally, ophthalmically, orally, rectally, sublabially, sublingually, subcutaneously, topically, and transdermally; and/or (ii) with a frequency selected from the group consisting of four times daily, three times daily, twice daily, once daily, once every two days, once every three days, once every four days, once every five days, once every six days, once a week, once every eight days, once every nine days, once every ten days, once every two weeks, once every three weeks, and once a month; and/or (iii) at a dosage (based on subject weight) selected from the group consisting of 0.1 μg/kg, 0.5 μg/kg, 1 μg/kg, 1.5 μg/kg, 2.0 μg/kg, 2.5 μg/kg, 3.0 μg/kg, 3.5 μg/kg, 4.0 μg/kg, 4.5 μg/kg, 5.0 μg/kg, 10 μg/kg, 15 μg/kg, 20 μg/kg, 25 μg/kg, 30 μg/kg, 35 μg/kg, 40 μg/kg, 45 μg/kg, 50 μg/kg, 55 μg/kg, 60 μg/kg, 65 μg/kg, 70 μg/kg, 75 μg/kg, 80 μg/kg, 85 μg/kg, 90 μg/kg, 95 μg/kg, 100 μg/kg, 150 μg/kg, 200 μg/kg, 250 μg/kg, 300 μg/kg, 350 μg/kg, 400 μg/kg, 450 μg/kg, 500 μg/kg, 550 μg/kg, 600 μg/kg, 650 μg/kg, 700 μg/kg, 750 μg/kg, 800 μg/kg, 850 μg/kg, 900 μg/kg, 1 mg/kg, 1.5 mg/kg, 2.0 mg/kg, 2.5 mg/kg, 3 mg/kg, 3.5 mg/kg, 4.0 mg/kg, 4.5 mg/kg, 5 mg/kg, 5.5 mg/kg, 6 mg/kg, 6.5 mg/kg, 7 mg/kg, 7.5 mg/kg, 8 mg/kg, 8.5 mg/kg, 9 mg/kg, 9.5 mg/kg, 10 mg/kg 10.5 mg/kg, 11 mg/kg, 11.5 mg/kg, 12 mg/kg, 12.5 mg/kg, 13 mg/kg, 13.5 mg/kg, 14 mg/kg, 14.5 mg/kg, 15 mg/kg, 16 mg/kg, 17 mg/kg, 18 mg/kg, 19 mg/kg, 20 mg/kg, 21 mg/kg, 22 mg/kg, 23 mg/kg, 24 mg/kg, 25 mg/kg, 30 mg/kg, 35 mg/kg, 40 mg/kg, 45 mg/kg, 50 mg/kg, 55 mg/kg, 60 mg/kg, 65 mg/kg, 70 mg/kg, 75 mg/kg, 80 mg/kg, 85 mg/kg, 90 mg/kg, 95 mg/kg, 100 mg/kg, 105 mg/kg, 110 mg/kg, 115 mg/kg, 120 mg/kg, 125 mg/kg, 130 mg/kg, 135 mg/kg, 140 mg/kg, 145 mg/kg, 150 mg/kg, 155 mg/kg, 160 mg/kg, 165 mg/kg, 170 mg/kg, 175 mg/kg, 180 mg/kg, 185 mg/kg, 190 mg/kg, 195 mg/kg, 200 mg/kg, and any fraction or integer in between any two of the preceding amounts.

In certain embodiments, an ovarian cancer subject, as described herein, may have an ovarian cancer selected from the group consisting of a Brenner tumor, a carcinoma, a choriocarinoma, a clear cell carcinoma, a dysgerminoma, an embryonal carcinoma, an endometrioid borderline neoplasm, an endometrioid carcinoma, an epithelial cancer, a fallopian tube cancer, a fibroma, a fibrothecoma, a granulosa cell tumor, a granulosa-theca tumor, a Krukenberg tumor, a mucinous borderline neoplasm, a mucinous carcinoma, a mucinous cystadenoma, an ovarian sarcoma, a primary peritoneal carcinoma, a serous carcinoma, a serous cystadenoma, a serous borderline neoplasm, a Sertoli cell tumor, a Sertoli-Leydig cell tumor, a small cell carcinoma, a teratoma, a thecoma, or a yolk sac tumor.

In some embodiments, an ovarian cancer subject, as described herein, may have an abnormal expression of one or more antigens compared to an equivalent subject that does not have ovarian cancer. As used herein, the term “equivalent subject,” refers to a subject of the age range of the ovarian cancer subject. For purposes of disclosure, the term “antigen,” when used with respect to abnormal expression, may define one or more CD antigens, such as, for example, CD15, CD44, CD70, CD133, as well as any CD antigen that has an abnormal expression in an ovarian cancer disclosed herein that would be readily envisaged by one skilled in the art. In certain embodiments, abnormal expression of an antigen may define an abnormal expression of CD15, an abnormal expression of CD44, an abnormal expression of CD70, an abnormal expression of CD133, an abnormal expression of a CD antigen that has an abnormal expression in an ovarian cancer disclosed herein that would be readily envisaged by one skilled in the art, and any combination thereof. In some embodiments, an “abnormal expression,” can refer to a reduced expression of an antigen, as described herein. In certain embodiments, an “abnormal expression,” can refer to an overexpression of an antigen, as described herein. Whether “abnormal expression,” refers to a reduced expression of an antigen or an overexpression of an antigen would be immediately envisaged by the skilled artisan when viewing the term in the context of the particular antigen disclosed, as well as the ovarian cancer to which the abnormal expression of antigen refers to. For example, in the context of CD44 and epithelial ovarian cancer, one skilled in the art would immediately envisage that “abnormal expression” refers to an overexpression of CD44. Treatment, amelioration, and/or reduction in the severity of an ovarian cancer or one or more symptoms thereof may be characterized by a restoration to a normal level of expression of one or more antigens from their abnormal state, as described herein. For example, in the context of CD44 and epithelial ovarian cancer, treatment, amelioration, and/or reduction in the severity of an ovarian cancer or one or more symptoms thereof may be characterized by a reduced expression of CD44.

In certain embodiments, an ovarian cancer subject, as described herein, may have an abnormal expression of one or more proteins compared to an equivalent subject that does not have ovarian cancer. For purposes of disclosure, the term “protein,” when used with respect to abnormal expression, may define one or more proteins and/or enzymes, such as, for example, ALDH1A3, mTOR, p-mTOR, TSC-2, AKT, pAKT, RPS6, p-RPS6, Syp, VEGF, PIGF, as well as any protein or enzyme that has an abnormal expression in an ovarian cancer disclosed herein that would be readily envisaged by one skilled in the art. In certain embodiments, abnormal expression of a protein may define an abnormal expression of ALDH1A3, an abnormal expression of mTOR, an abnormal expression of p-mTOR, an abnormal expression of TSC-2, an abnormal expression of AKT, an abnormal expression of pAKT, an abnormal expression of RPS6, an abnormal expression of p-RPS6, an abnormal expression of Syp, an abnormal expression of VEGF, an abnormal expression of PIGF, an abnormal expression of a protein or enzyme that has an abnormal expression in an ovarian cancer disclosed herein that would be readily envisaged by one skilled in the art, and any combination thereof. In some embodiments, an “abnormal expression,” can refer to a reduced expression of a protein, as described herein. In certain embodiments, an “abnormal expression,” can refer to an overexpression of a protein, as described herein. Whether “abnormal expression,” refers to a reduced expression of a protein or an overexpression of a protein would be immediately envisaged by the skilled artisan when viewing the term in the context of the particular protein disclosed, as well as the ovarian cancer to which the abnormal expression of protein refers to. For example, in the context of mTOR and AKT and serous carcinomas, one skilled in the art would immediately envisage that “abnormal expression” refers to an overexpression of mTOR and/or AKT. Treatment, amelioration, and/or reduction in the severity of an ovarian cancer or one or more symptoms thereof may be characterized by a restoration to a normal level of expression of one or more proteins from their abnormal state, as described herein. For example, in the context of mTOR and/or AKT and serous carcinomas, treatment, amelioration, and/or reduction in the severity of an ovarian cancer or one or more symptoms thereof may be characterized by a reduced expression of mTOR and/or AKT.

In certain embodiments, an ovarian cancer subject, as described herein, may have an increased expression of one or more NOS isoforms compared to an equivalent subject that does not have ovarian cancer. For purposes of disclosure, the term “NOS isoform,” refers to nNOS, iNOS, and/or eNOS. In certain embodiments, an ovarian cancer subject, as described herein, may have an increased expression of nNOS compared to an equivalent subject that does not have ovarian cancer. In some embodiments, an ovarian cancer subject, as described herein, may have an increased expression of iNOS compared to an equivalent subject that does not have ovarian cancer. In certain embodiments, an ovarian cancer subject, as described herein, may have an increased expression of eNOS compared to an equivalent subject that does not have ovarian cancer. In some embodiments, an ovarian cancer subject, as described herein, may have an increased expression of nNOS and iNOS compared to an equivalent subject that does not have ovarian cancer. In certain embodiments, an ovarian cancer subject, as described herein, may have an increased expression of nNOS and eNOS compared to an equivalent subject that does not have ovarian cancer. In some embodiments, an ovarian cancer subject, as described herein, may have an increased expression of iNOS and eNOS compared to an equivalent subject that does not have ovarian cancer. In certain embodiments, an ovarian cancer subject, as described herein, may have an increased expression of nNOS, iNOS, and eNOS compared to an equivalent subject that does not have ovarian cancer. Whether a “NOS isoform,” has an increased expression would be immediately envisaged by the skilled artisan when viewing the term in the context of the particular NOS isoform disclosed, as well as the ovarian cancer to which the increased expression of NOS isoform refers to. For example, in the context of epithelial ovarian cancer, one skilled in the art would immediately envisage that iNOS has an increased expression. Treatment, amelioration, and/or reduction in the severity of an ovarian cancer or one or more symptoms thereof may be characterized by a reduced expression of one or more NOS isoforms from their increased state, as described herein. For example, in the context of epithelial ovarian cancer, treatment, amelioration, and/or reduction in the severity of an ovarian cancer or one or more symptoms thereof may be characterized by a reduced expression of iNOS. For purposes of disclosure, reduction/inhibition of the expression of one or more NOS isoforms may be characterized by a reduction in expression of one or more NOS isoforms by about at least 10%, about at least 20%, about at least 30%, or about at least 40%. In some embodiments, reduction/inhibition of the expression of one or more NOS isoforms may further be characterized by a lack reduction of other, non-implicated NOS isoforms, such as, for example, no more than about 10%, no more than about 5%, no more than about 2%, or no more than about 1% reduction/inhibition of the expression of non-implicated NOS isoforms. As used herein, the term “non-implicated NOS isoforms,” refers to NOS isoforms that do not have an increased expression in a particular ovarian cancer. By way of example, in the context of epithelial ovarian cancer, if iNOS has an increased expression, nNOS and eNOS would define “non-implicated” NOS isoforms. Whether a NOS isoform is a “non-implicated NOS isoform,” would be immediately envisaged by the skilled artisan when viewing the term in the context of the particular NOS isoform disclosed, as well as the ovarian cancer.

In some embodiments, an ovarian cancer subject, as described herein, may have an elevated level of one or more NO-indicators compared to an equivalent subject that does not have ovarian cancer. As used herein, the term “NO-indicator,” can refer to 3-nitrotyrosine (3-Ntyr), NADPH-diaphorase, total nitrite concentration, total nitrate concentration, total nitrite and nitrate concentration, as well as any NO-indicator that has an elevated level in an ovarian cancer disclosed herein that would be readily envisaged by one skilled in the art. For example, in the context of epithelial ovarian cancer, one skilled in the art would immediately envisage that 3-Ntyr can be present at an elevated level. Treatment, amelioration, and/or reduction in the severity of an ovarian cancer or one or more symptoms thereof may be characterized by a reduction in the level of one or more NO-indicators. For example, in the context of epithelial ovarian cancer, treatment, amelioration, and/or reduction in the severity of an ovarian cancer or one or more symptoms thereof may be characterized by a reduction in the level of 3-Ntyr.

In certain embodiments, a NOS inhibitor composition, as described herein, may be administered as a suitable dosage form, as described herein, to treat, ameliorate, and/or reduce the severity of an ovarian cancer or one or more symptoms thereof, as an alternative to a conventional therapy for treating, ameliorating, and/or reducing the severity of an ovarian cancer or one or more symptoms thereof. For purposes of disclosure, a “conventional therapy,” as described herein, may refer to a treatment that would be recommended by one skilled in the art to treat an ovarian cancer, and may define one or more of radiation, chemotherapy, immunotherapy, cell-based therapy, gene therapy, and the like—one skilled in the art would immediately envisage a “conventional therapy” based on the particular ovarian cancer disclosed, as well as characteristics of an ovarian cancer subject, such as, for example, the subject's age, stage of the ovarian cancer, genotype of the ovarian cancer, and the like. Treatment, amelioration, and/or reduction in the severity of an ovarian cancer or one or more symptoms thereof may be characterized by an improved outcome compared to a conventional therapy, as described herein. For purposes of the disclosure, an “improved outcome” may define any one of the following: (i) a larger reduction in tumor volume and/or mass compared to a subject administered an equivalent dose of a conventional therapy; (ii) an improvement in the expected rate of survival of a subject, at, for example, 1 month, 3 months, 6 months, 9 months, 1 year, 2 years, 3 years, 5 years, 10 years, or more, compared to the expected rate of survival of a subject administered an equivalent dose of a conventional therapy; (iii) a reduction in the prevalence of side effects resulting from the NOS inhibitor composition compared to a subject administered an equivalent dose of a conventional therapy; (iv) a reduction in the severity of side effects resulting from the NOS inhibitor composition compared to a subject administered an equivalent dose of a conventional therapy; (v) a reduction in the dosage required to achieve an equivalent reduction in tumor volume compared to a subject administered a conventional therapy; (vi) a reduction in the dosage required to achieve an equivalent expected rate of survival of a subject compared to a subject administered a conventional therapy; (vii) a reduction in the likelihood of a recurrence of the ovarian cancer occurring in a subject compared to a subject administered an equivalent dose of a conventional therapy; (viii) a reduction in the likelihood of a metastasis occurring in a subject compared to a subject administered an equivalent dose of a conventional therapy; (ix) a reduction in the time required to be at a medical facility for treatment compared to a subject administered an equivalent dose of a conventional therapy; (x) a reduction in the cost associated with treatment compared to a subject administered an equivalent dose of a conventional therapy; as well as any other improved outcome not expressly recited that would be readily envisaged by one skilled in the art.

In some embodiments, a NOS inhibitor composition, as described herein, may be administered as a suitable dosage form, as described herein, to treat, ameliorate, and/or reduce the severity of an ovarian cancer or one or more symptoms thereof, wherein the NOS inhibitor composition comprises a first chemical NOS inhibitor and a second chemical NOS inhibitor. In certain embodiments, the first chemical NOS inhibitor may be administered concurrently with the second chemical NOS inhibitor. In some embodiments, the first chemical NOS inhibitor may be administered prior to the second chemical NOS inhibitor. In certain embodiments, the first chemical NOS inhibitor may be administered subsequently to the second chemical NOS inhibitor. Treatment, amelioration, and/or reduction in the severity of an ovarian cancer or one or more symptoms thereof may be characterized by an improved outcome compared an alternative treatment. As used herein, the term “alternative treatment,” may define one or more of a conventional therapy, as described herein, a NOS inhibitor composition comprising a single chemical NOS inhibitor, as described herein, and a NOS inhibitor composition comprising a single biological NOS inhibitor, as described herein. For purposes of the disclosure, an “improved outcome” may define any one of the following: (i) a larger reduction in tumor volume and/or mass compared an alternative treatment; (ii) an improvement in the expected rate of survival of a subject, at, for example, 1 month, 3 months, 6 months, 9 months, 1 year, 2 years, 3 years, 5 years, 10 years, or more, compared to the expected rate of survival of a subject administered an alternative treatment; (iii) a reduction in the prevalence of side effects resulting from the NOS inhibitor composition comprising at least two chemical NOS inhibitors compared to a subject administered an alternative treatment; (iv) a reduction in the severity of side effects resulting from the NOS inhibitor composition comprising at least two chemical NOS inhibitors compared to a subject administered an alternative treatment; (v) a reduction in the dosage required to achieve an equivalent reduction in tumor volume compared to a subject administered an alternative treatment; (vi) a reduction in the dosage required to achieve an equivalent expected rate of survival of a subject compared to a subject administered an alternative treatment; (vii) a reduction in the likelihood of a recurrence of the ovarian cancer in a subject compared to a subject administered an alternative treatment; (viii) a reduction in the likelihood of a metastasis occurring in a subject compared to a subject administered an alternative treatment; (ix) a reduction in the time required to be at a medical facility for treatment compared to a subject administered an alternative treatment; (x) a reduction in the cost associated with treatment compared to a subject administered an alternative treatment; as well as any other improved outcome not expressly recited that would be readily envisaged by one skilled in the art.

In certain embodiments, a NOS inhibitor composition, as described herein, may be used to treat, ameliorate, and/or reduce the severity of an ovarian cancer or one or more symptoms thereof, wherein the NOS inhibitor composition comprises at least one chemical NOS inhibitor and at least one biological NOS inhibitor. In some embodiments, the at least one chemical NOS inhibitor may be administered concurrently with the at least one biological NOS inhibitor. In certain embodiments, the at least one chemical NOS inhibitor may be administered prior to the at least one biological NOS inhibitor. In some embodiments, the at least one chemical NOS inhibitor may be administered subsequently to the at least one biological NOS inhibitor. Treatment, amelioration, and/or reduction in the severity of an ovarian cancer or one or more symptoms thereof may be characterized by an improved outcome compared an alternative treatment. As used herein, the term “alternative treatment,” may define one or more of a conventional therapy, as described herein, a NOS inhibitor composition comprising a single chemical NOS inhibitor, as described herein, and a NOS inhibitor composition comprising a single biological NOS inhibitor, as described herein. For purposes of the disclosure, an “improved outcome” may define any one of the following: (i) a larger reduction in tumor volume and/or mass compared an alternative treatment; (ii) an improvement in the expected rate of survival of a subject, at, for example, 1 month, 3 months, 6 months, 9 months, 1 year, 2 years, 3 years, 5 years, 10 years, or more, compared to the expected rate of survival of a subject administered an alternative treatment; (iii) a reduction in the prevalence of side effects resulting from the NOS inhibitor composition comprising at least one chemical NOS inhibitor and at least one biological NOS inhibitor compared to a subject administered an alternative treatment; (iv) a reduction in the severity of side effects resulting from the NOS inhibitor composition comprising at least one chemical NOS inhibitor and at least one biological NOS inhibitor compared to a subject administered an alternative treatment; (v) a reduction in the dosage required to achieve an equivalent reduction in tumor volume compared to a subject administered an alternative treatment; (vi) a reduction in the dosage required to achieve an equivalent expected rate of survival of a subject compared to a subject administered an alternative treatment; (vii) a reduction in the likelihood of a recurrence of the ovarian cancer in a subject compared to a subject administered an alternative treatment; (viii) a reduction in the likelihood of a metastasis occurring in a subject compared to a subject administered an alternative treatment; (ix) a reduction in the time required to be at a medical facility for treatment compared to a subject administered an alternative treatment; (x) a reduction in the cost associated with treatment compared to a subject administered an alternative treatment; as well as any other improved outcome not expressly recited that would be readily envisaged by one skilled in the art.

In some embodiments, a NOS inhibitor composition, as described herein, may be combined with a conventional therapy, as described herein, to treat, ameliorate, and/or reduce the severity of an ovarian cancer or one or more symptoms thereof. In certain embodiments, the NOS inhibitor composition may be administered concurrently with the conventional therapy. In some embodiments, the NOS inhibitor composition may be administered prior to the conventional therapy. In certain embodiments, the NOS inhibitor composition may be administered subsequently to the conventional therapy. Treatment, amelioration, and/or reduction in the severity of an ovarian cancer or one or more symptoms thereof may be characterized by an improved outcome compared an alternative treatment. As used herein, the term “alternative treatment,” may define one or more of a conventional therapy, as described herein, a NOS inhibitor composition comprising a single chemical NOS inhibitor, as described herein, and a NOS inhibitor composition comprising a single biological NOS inhibitor, as described herein. For purposes of the disclosure, an “improved outcome” may define any one of the following: (i) a larger reduction in tumor volume and/or mass compared an alternative treatment; (ii) an improvement in the expected rate of survival of a subject, at, for example, 1 month, 3 months, 6 months, 9 months, 1 year, 2 years, 3 years, 5 years, 10 years, or more, compared to the expected rate of survival of a subject administered an alternative treatment; (iii) a reduction in the prevalence of side effects resulting from the NOS inhibitor composition and conventional therapy compared to a subject administered an alternative treatment; (iv) a reduction in the severity of side effects resulting from the NOS inhibitor composition and conventional therapy compared to a subject administered an alternative treatment; (v) a reduction in the dosage required to achieve an equivalent reduction in tumor volume compared to a subject administered an alternative treatment; (vi) a reduction in the dosage required to achieve an equivalent expected rate of survival of a subject compared to a subject administered an alternative treatment; (vii) a reduction in the likelihood of a recurrence of the ovarian cancer in a subject compared to a subject administered an alternative treatment; (viii) a reduction in the likelihood of a metastasis occurring in a subject compared to a subject administered an alternative treatment; (ix) a reduction in the time required to be at a medical facility for treatment compared to a subject administered an alternative treatment; (x) a reduction in the cost associated with treatment compared to a subject administered an alternative treatment; as well as any other improved outcome not expressly recited that would be readily envisaged by one skilled in the art.

In some embodiments, a “conventional therapy,” as that term is used with respect to combined administration with a NOS inhibitor composition, may refer to a conventional chemotherapy. For purposes of the disclosure, a “conventional chemotherapy,” as described herein may be a compound selected from the group consisting of abemaciclib, abiraterone acetate, acalabrutinib, adagrasib, afatinib dimaleate, albumin-bound paclitaxel, albumin-bound rapamycin (Sirolimus Protein-Bound Particles), alectinib, alpelisib, amifostine, aminolevulinic acid hydrochloride, anastrozole, apalutamide, aprepitant, arsenic trioxide, asciminib hydrochloride, avapritinib, axitinib, azacytidine, belinostat, belzutifan, bendamustine hydrochloride, bexarotene, bicalutamide, binimetinib, bleomycin, bleomycin sulfate, bortezomib, bosutinib, brigatinib, busulfan, cabazitaxel, cabozantinib-S-malate, capecitabine, capmatinib hydrochloride, carboplatin, carfilzomib, carmustine, cedazuridine, ceritinib, chlorambucil, cisplatin, cladribine, clofarabine, cobimetinib fumarate, copanlisib hydrochloride, crizotinib, cyclophosphamide, cytarabine, dabrafenib mesylate, dacarbazine, dacomitinib, dactinomycin, darolutamide, dasatinib, daunorubicin hydrochloride, decitabine, defibrotide sodium, degarelix, dexamethasone, dexrazoxane hydrochloride, docetaxel, doxorubicin hydrochloride, duvelisib, elacestrant dihydrochloride, eltrombopag olamine, enasidenib mesylate, encorafenib, entrectinib, enzalutamide, epirubicin hydrochloride, erdafitinib, eribulin mesylate, erlotinib hydrochloride, etoposide, etoposide phosphate, everolimus, exemestane, fedratinib hydrochloride, fludarabine phosphate, fluorouracil, flutamide, folinic acid, fostamatinib disodium, fulvestrant, futibatinib, gefitinib, gemcitabine hydrochloride, gilteritinib fumarate, glasdegib maleate, goserelin acetate, granisetron, granisetron hydrochloride, hydroxyurea, ibrutinib, idarubicin hydrochloride, idelalisib, ifosfamide, imatinib mesylate, imiquimod, infigratinib phosphate, iobenguane I-131, irinotecan hydrochloride, ivosidenib, ixabepilone, ixazomib citrate, lanreotide acetate, lapatinib ditosylate, larotrectinib sulfate, lenalidomide, lenvatinib mesylate, letrozole, leucovorin calcium, leuprolide acetate, liposomal cytarabine, liposomal doxorubicin hydrochloride, liposomal irinotecan hydrochloride, lomustine, lorlatinib, lurbinectedin, Lu-177 dotatate, Lu-177 vipivotide tetraxetan, mechlorethamine hydrochloride, megestrol acetate, melphalan, melphalan hydrochloride, mercaptopurine, mesna, methotrexate, methotrexate sodium, methylnaltrexone bromide, midostaurin, mitomycin, mitoxantrone hydrochloride, mobocertinib succinate, nelarabine, neratinib maleate, netupitant hydrochloride, nilutamide, nilotinib, niraparib tosylate monohydrate, olaparib, olutasidenib, omacetaxine mepesuccinate, ondansetron hydrochloride, osimertinib mesylate, oxaliplatin, paclitaxel, pacritinib citrate, palbociclib, palonosetron hydrochloride, pamidronate disodium, pazopanib hydrochloride, pemetrexed disodium, pemigatinib, pexidartinib hydrochloride, pirtobrutinib, plerixafor, pomalidomide, ponatinib hydrochloride, pralatrexate, pralsetinib, prednisone, procarbazine hydrochloride, propranolol hydrochloride, Ra-223 dichloride, raloxifene hydrochloride, regorafenib, relugolix, ribociclib, ripretinib, rolapitant hydrochloride, romidepsin, rucaparib camsylate, ruxolitinib phosphate, selinexor, selpercatinib, selumetinib sulfate, sonidegib, sorafenib tosylate, sotorasib, sunitinib malate, talazoparib tosylate, talc, tamoxifen citrate, tazemetostat hydrobromide, temozolomide, temsirolimus, tepotinib hydrochloride, thalidomide, thioguanine, thiotepa, tipiracil hydrochloride, tivozanib hydrochloride, topotecan hydrochloride, toremifene, trabectedin, trametinib dimethyl sulfoxide, tretinoin, trifluridine hydrochloride, tucatinib, uridine triacetate, valrubicin, vandetanib, vemurafenib, venetoclax, vinblastine sulfate, vincristine sulfate, vinorelbine tartrate, vismodegib, vorinostat, zanubrutinib, zoledronic acid, and any combination thereof. One skilled in the art would immediately envisage an appropriate “conventional chemotherapy” based on the particular ovarian cancer disclosed, as well as characteristics of an ovarian cancer subject, such as, for example, the subject's age, stage of the ovarian cancer, genotype of the ovarian cancer, and the like.

In certain embodiments, a “conventional therapy,” as that term is used with respect to combined administration with a NOS inhibitor composition, may refer to a conventional immunotherapy. For purposes of the disclosure, a “conventional immunotherapy,” as described herein may be an immunotherapy, cell-based therapy, and/or gene-based therapy selected from the group consisting of ado-trastuzumab emtansine, aldesleukin, alemtuzumab, amivantamab-vmjw, asparaginase Erwinia chrysanthemi, asparaginase Erwinia chrysanthemi (recombinant)-rywn, atezolizumab, avelumab, axicabtagene ciloleucel, bevacizumab, blinatumomab, brentuximab vedotin, brexucabtagene autoleucel, calaspargase pegol-mknl, camrelizumab, caplacizumab-yhdp, cemiplimab-rwlc, cetuximab, ciltacabtagene autoleucel, daratumumab, daratumumab and hyaluronidase-fihj, darbepoetin alfa, denileukin diftitox, denosumab, dinutuximab, dostarlimab-gxly, durvalumab, elotuzumab, emapalumab-lzsg, enfortumab vedotin-ejfv, epoetin alfa, fam-trastuzumab deruxtecan-nxki, filgrastim, gemtuzumab ozogamicin, glucarpidase, ibritumomab tiuxetan, idecabtagene vicleucel, inotuzumab ozogamicin, ipilimumab, isatuximab-irfc, lisocabtagene maraleucel, loncastuximab tesirine-lpyl, luspatercept-aamt, margetuximab-cmkb, mirvetuximab soravtansine-gynx, mogamulizumab-kpkc, mosunetuzumab-axgb, moxetumomab pasudotox-tdfk, nadofaragene firadenovec-vncg, naxitamab-gqgk, necitumumab, nivolumab, nivolumab and relatlimab-rmbw, obinutuzumab, ofatumumab, palifermin, panitumumab, pegaspargase, pegfilgrastim, pembrolizumab, pertuzumab, pertuzumab, trastuzumab, and hyaluronidase-zzxf, polatuzumab vedotin-piiq, ramucirumab, rasburicase, ravulizumab-cwvz, recombinant HPV bivalent vaccine, recombinant HPV nonavalent vaccine, recombinant HPV quadrivalent vaccine, recombinant interferon alfa-2b, rituximab, rituximab and hyaluronidase human, romiplostim, ropeginterferon alfa-2b-njft, sacituzumab govitecan-hziy, siltuximab, sintilimab, sipuleucel-T, tafasitamab-cxix, tagraxofusp-erzs, talimogene laherparepvec, tebentafusp-tebn, teclistamab-cqyv, tisagenlecleucel, tislelizumab, tisotumab vedotin-tftv, tocilizumab, toripalimab, trastuzumab, trastuzumab and hyaluronidase-oysk, tremelimumab-actl, ziv-aflibercept, and any combination thereof. One skilled in the art would immediately envisage an appropriate “conventional immunotherapy” based on the particular ovarian cancer disclosed, as well as characteristics of an ovarian cancer subject, such as, for example, the subject's age, stage of the ovarian cancer, genotype of the ovarian cancer, and the like.

In certain embodiments, a NOS inhibitor composition, as described herein, may be administered as a suitable dosage form, as described herein, prior to, or alternatively, following a surgery to remove or resect an ovarian cancer.

In some embodiments, a NOS inhibitor composition, as described herein, may be administered as a suitable dosage form, as described herein, to restore and/or maintain healthy and/or optimal expression levels of one or more antigens, as described herein, such as, for example, CD15, CD44, CD70, CD133, as well as any CD antigen that has an abnormal expression in an ovarian cancer disclosed herein that would be readily envisaged by one skilled in the art, in a subject. In certain embodiments, the subject may have an ovarian cancer or one or more symptoms thereof. As used herein, the terms “healthy” and “optimal” refer to expression levels of one or more antigens that would be typical of an equivalent subject that does not have ovarian cancer.

In certain embodiments, a NOS inhibitor composition, as described herein, may be administered as a suitable dosage form, as described herein, to restore and/or maintain healthy and/or optimal expression levels of one or more proteins, as described herein, such as, for example, ALDH1A3, mTOR, p-mTOR, TSC-2, AKT, pAKT, RPS6, p-RPS6, Syp, VEGF, PIGF, as well as any protein or enzyme that has an abnormal expression in an ovarian cancer disclosed herein that would be readily envisaged by one skilled in the art, in a subject. In certain embodiments, the subject may have an ovarian cancer or one or more symptoms thereof. As used herein, the terms “healthy” and “optimal” refer to expression levels of one or more proteins that would be typical of an equivalent subject that does not have ovarian cancer.

In some embodiments, a NOS inhibitor composition, as described herein, may be administered as a suitable dosage form, as described herein, to restore and/or maintain healthy and/or optimal expression levels of one or more NOS isoforms, as described herein, such as, for example, nNOS, iNOS, and eNOS, in a subject. In certain embodiments, the subject may have an ovarian cancer or one or more symptoms thereof. As used herein, the terms “healthy” and “optimal” refer to expression levels of one or more NOS isoforms that would be typical of an equivalent subject that does not have ovarian cancer.

In certain embodiments, a NOS inhibitor composition, as described herein, may be administered as a suitable dosage form, as described herein, to restore and/or maintain healthy and/or optimal levels of one or more NO-indicators, as described herein, such as, for example, 3-Ntyr, NADPH-diaphorase, total nitrite concentration, total nitrate concentration, total nitrite and nitrate concentration, as well as any NO-indicator that has an elevated level in an ovarian cancer disclosed herein that would be readily envisaged by one skilled in the art, in a subject. In certain embodiments, the subject may have an ovarian cancer or one or more symptoms thereof. As used herein, the terms “healthy” and “optimal” refer to levels of one or more NO-indicators that would be typical of an equivalent subject that does not have ovarian cancer.

In certain embodiments, a NOS inhibitor composition, as described herein, may be administered as a suitable dosage form, as described herein, to treat, ameliorate, and/or reduce the severity of a cervical cancer or one or more symptoms thereof in a subject (“cervical cancer subject”). As used herein, the term “cervical cancer” refers to a cancer that occurs and/or originates in the cervix.

In some embodiments, a NOS inhibitor composition comprising 7-NI and/or a salt thereof may be administered as a suitable dosage form, as described herein, to treat, ameliorate, and/or reduce the severity of cervical cancer or one or more symptoms thereof in a cervical cancer subject. In certain embodiments, an nNOS inhibitor composition comprising 7-NI and/or a salt thereof may be administered to a cervical cancer subject to treat, ameliorate, and/or reduce the severity of cervical cancer or one or more symptoms thereof in the cervical cancer subject: (i) via a route selected from the group consisting of buccally, inhalation, intraarterially, intraarticularly, intracardially, intracerebroventricularly, intradermally, intramedullary, intramuscularly, intraperitoneally, intrathecally, intravenously, intraventricularly, nasally, ophthalmically, orally, rectally, sublabially, sublingually, subcutaneously, topically, and transdermally; and/or (ii) with a frequency selected from the group consisting of four times daily, three times daily, twice daily, once daily, once every two days, once every three days, once every four days, once every five days, once every six days, once a week, once every eight days, once every nine days, once every ten days, once every two weeks, once every three weeks, and once a month; and/or (iii) at a dosage (based on subject weight) selected from the group consisting of 0.1 μg/kg, 0.5 μg/kg, 1 μg/kg, 1.5 μg/kg, 2.0 μg/kg, 2.5 μg/kg, 3.0 μg/kg, 3.5 μg/kg, 4.0 μg/kg, 4.5 μg/kg, 5.0 μg/kg, 10 μg/kg, 15 μg/kg, 20 μg/kg, 25 μg/kg, 30 μg/kg, 35 μg/kg, 40 μg/kg, 45 μg/kg, 50 μg/kg, 55 μg/kg, 60 μg/kg, 65 μg/kg, 70 μg/kg, 75 μg/kg, 80 μg/kg, 85 μg/kg, 90 μg/kg, 95 μg/kg, 100 μg/kg, 150 μg/kg, 200 μg/kg, 250 μg/kg, 300 μg/kg, 350 μg/kg, 400 μg/kg, 450 μg/kg, 500 μg/kg, 550 μg/kg, 600 μg/kg, 650 μg/kg, 700 μg/kg, 750 μg/kg, 800 μg/kg, 850 μg/kg, 900 μg/kg, 1 mg/kg, 1.5 mg/kg, 2.0 mg/kg, 2.5 mg/kg, 3 mg/kg, 3.5 mg/kg, 4.0 mg/kg, 4.5 mg/kg, 5 mg/kg, 5.5 mg/kg, 6 mg/kg, 6.5 mg/kg, 7 mg/kg, 7.5 mg/kg, 8 mg/kg, 8.5 mg/kg, 9 mg/kg, 9.5 mg/kg, 10 mg/kg 10.5 mg/kg, 11 mg/kg, 11.5 mg/kg, 12 mg/kg, 12.5 mg/kg, 13 mg/kg, 13.5 mg/kg, 14 mg/kg, 14.5 mg/kg, 15 mg/kg, 16 mg/kg, 17 mg/kg, 18 mg/kg, 19 mg/kg, 20 mg/kg, 21 mg/kg, 22 mg/kg, 23 mg/kg, 24 mg/kg, 25 mg/kg, 30 mg/kg, 35 mg/kg, 40 mg/kg, 45 mg/kg, 50 mg/kg, 55 mg/kg, 60 mg/kg, 65 mg/kg, 70 mg/kg, 75 mg/kg, 80 mg/kg, 85 mg/kg, 90 mg/kg, 95 mg/kg, 100 mg/kg, 105 mg/kg, 110 mg/kg, 115 mg/kg, 120 mg/kg, 125 mg/kg, 130 mg/kg, 135 mg/kg, 140 mg/kg, 145 mg/kg, 150 mg/kg, 155 mg/kg, 160 mg/kg, 165 mg/kg, 170 mg/kg, 175 mg/kg, 180 mg/kg, 185 mg/kg, 190 mg/kg, 195 mg/kg, 200 mg/kg, and any fraction or integer in between any two of the preceding amounts.

In certain embodiments, a NOS inhibitor composition comprising 3-Br-7-NI and/or a salt thereof may be administered as a suitable dosage form, as described herein, to treat, ameliorate, and/or reduce the severity of cervical cancer or one or more symptoms thereof in a cervical cancer subject. In some embodiments, an nNOS inhibitor composition comprising 3-Br-7-NI and/or a salt thereof may be administered to a cervical cancer subject to treat, ameliorate, and/or reduce the severity of cervical cancer or one or more symptoms thereof in the cervical cancer subject: (i) via a route selected from the group consisting of buccally, inhalation, intraarterially, intraarticularly, intracardially, intracerebroventricularly, intradermally, intramedullary, intramuscularly, intraperitoneally, intrathecally, intravenously, intraventricularly, nasally, ophthalmically, orally, rectally, sublabially, sublingually, subcutaneously, topically, and transdermally; and/or (ii) with a frequency selected from the group consisting of four times daily, three times daily, twice daily, once daily, once every two days, once every three days, once every four days, once every five days, once every six days, once a week, once every eight days, once every nine days, once every ten days, once every two weeks, once every three weeks, and once a month; and/or (iii) at a dosage (based on subject weight) selected from the group consisting of 0.1 μg/kg, 0.5 μg/kg, 1 μg/kg, 1.5 μg/kg, 2.0 μg/kg, 2.5 μg/kg, 3.0 μg/kg, 3.5 μg/kg, 4.0 μg/kg, 4.5 μg/kg, 5.0 μg/kg, 10 μg/kg, 15 μg/kg, 20 μg/kg, 25 μg/kg, 30 μg/kg, 35 μg/kg, 40 μg/kg, 45 μg/kg, 50 μg/kg, 55 μg/kg, 60 μg/kg, 65 μg/kg, 70 μg/kg, 75 μg/kg, 80 μg/kg, 85 μg/kg, 90 μg/kg, 95 μg/kg, 100 μg/kg, 150 μg/kg, 200 μg/kg, 250 μg/kg, 300 μg/kg, 350 μg/kg, 400 μg/kg, 450 μg/kg, 500 μg/kg, 550 μg/kg, 600 μg/kg, 650 μg/kg, 700 μg/kg, 750 μg/kg, 800 μg/kg, 850 μg/kg, 900 μg/kg, 1 mg/kg, 1.5 mg/kg, 2.0 mg/kg, 2.5 mg/kg, 3 mg/kg, 3.5 mg/kg, 4.0 mg/kg, 4.5 mg/kg, 5 mg/kg, 5.5 mg/kg, 6 mg/kg, 6.5 mg/kg, 7 mg/kg, 7.5 mg/kg, 8 mg/kg, 8.5 mg/kg, 9 mg/kg, 9.5 mg/kg, 10 mg/kg 10.5 mg/kg, 11 mg/kg, 11.5 mg/kg, 12 mg/kg, 12.5 mg/kg, 13 mg/kg, 13.5 mg/kg, 14 mg/kg, 14.5 mg/kg, 15 mg/kg, 16 mg/kg, 17 mg/kg, 18 mg/kg, 19 mg/kg, 20 mg/kg, 21 mg/kg, 22 mg/kg, 23 mg/kg, 24 mg/kg, 25 mg/kg, 30 mg/kg, 35 mg/kg, 40 mg/kg, 45 mg/kg, 50 mg/kg, 55 mg/kg, 60 mg/kg, 65 mg/kg, 70 mg/kg, 75 mg/kg, 80 mg/kg, 85 mg/kg, 90 mg/kg, 95 mg/kg, 100 mg/kg, 105 mg/kg, 110 mg/kg, 115 mg/kg, 120 mg/kg, 125 mg/kg, 130 mg/kg, 135 mg/kg, 140 mg/kg, 145 mg/kg, 150 mg/kg, 155 mg/kg, 160 mg/kg, 165 mg/kg, 170 mg/kg, 175 mg/kg, 180 mg/kg, 185 mg/kg, 190 mg/kg, 195 mg/kg, 200 mg/kg, and any fraction or integer in between any two of the preceding amounts.

In some embodiments, a NOS inhibitor composition comprising L-NPA and/or a salt thereof, such as, for example, L-NPA hydrochloride, may be administered as a suitable dosage form, as described herein, to treat, ameliorate, and/or reduce the severity of cervical cancer or one or more symptoms thereof in a cervical cancer subject. In certain embodiments, an nNOS inhibitor composition comprising L-NPA and/or a salt thereof, such as, for example, L-NPA hydrochloride may be administered to a cervical cancer subject to treat, ameliorate, and/or reduce the severity of cervical cancer or one or more symptoms thereof in the cervical cancer subject: (i) via a route selected from the group consisting of buccally, inhalation, intraarterially, intraarticularly, intracardially, intracerebroventricularly, intradermally, intramedullary, intramuscularly, intraperitoneally, intrathecally, intravenously, intraventricularly, nasally, ophthalmically, orally, rectally, sublabially, sublingually, subcutaneously, topically, and transdermally; and/or (ii) with a frequency selected from the group consisting of four times daily, three times daily, twice daily, once daily, once every two days, once every three days, once every four days, once every five days, once every six days, once a week, once every eight days, once every nine days, once every ten days, once every two weeks, once every three weeks, and once a month; and/or (iii) at a dosage (based on subject weight) selected from the group consisting of 0.1 μg/kg, 0.5 μg/kg, 1 μg/kg, 1.5 μg/kg, 2.0 μg/kg, 2.5 μg/kg, 3.0 μg/kg, 3.5 μg/kg, 4.0 μg/kg, 4.5 μg/kg, 5.0 μg/kg, 10 μg/kg, 15 μg/kg, 20 μg/kg, 25 μg/kg, 30 μg/kg, 35 μg/kg, 40 μg/kg, 45 μg/kg, 50 μg/kg, 55 μg/kg, 60 μg/kg, 65 μg/kg, 70 μg/kg, 75 μg/kg, 80 μg/kg, 85 μg/kg, 90 μg/kg, 95 μg/kg, 100 μg/kg, 150 μg/kg, 200 μg/kg, 250 μg/kg, 300 μg/kg, 350 μg/kg, 400 μg/kg, 450 μg/kg, 500 μg/kg, 550 μg/kg, 600 μg/kg, 650 μg/kg, 700 μg/kg, 750 μg/kg, 800 μg/kg, 850 μg/kg, 900 μg/kg, 1 mg/kg, 1.5 mg/kg, 2.0 mg/kg, 2.5 mg/kg, 3 mg/kg, 3.5 mg/kg, 4.0 mg/kg, 4.5 mg/kg, 5 mg/kg, 5.5 mg/kg, 6 mg/kg, 6.5 mg/kg, 7 mg/kg, 7.5 mg/kg, 8 mg/kg, 8.5 mg/kg, 9 mg/kg, 9.5 mg/kg, 10 mg/kg 10.5 mg/kg, 11 mg/kg, 11.5 mg/kg, 12 mg/kg, 12.5 mg/kg, 13 mg/kg, 13.5 mg/kg, 14 mg/kg, 14.5 mg/kg, 15 mg/kg, 16 mg/kg, 17 mg/kg, 18 mg/kg, 19 mg/kg, 20 mg/kg, 21 mg/kg, 22 mg/kg, 23 mg/kg, 24 mg/kg, 25 mg/kg, 30 mg/kg, 35 mg/kg, 40 mg/kg, 45 mg/kg, 50 mg/kg, 55 mg/kg, 60 mg/kg, 65 mg/kg, 70 mg/kg, 75 mg/kg, 80 mg/kg, 85 mg/kg, 90 mg/kg, 95 mg/kg, 100 mg/kg, 105 mg/kg, 110 mg/kg, 115 mg/kg, 120 mg/kg, 125 mg/kg, 130 mg/kg, 135 mg/kg, 140 mg/kg, 145 mg/kg, 150 mg/kg, 155 mg/kg, 160 mg/kg, 165 mg/kg, 170 mg/kg, 175 mg/kg, 180 mg/kg, 185 mg/kg, 190 mg/kg, 195 mg/kg, 200 mg/kg, and any fraction or integer in between any two of the preceding amounts.

In certain embodiments, a NOS inhibitor composition comprising L-NIO and/or a salt thereof, such as, for example, L-NIO dihydrochloride, may be administered as a suitable dosage form, as described herein, to treat, ameliorate, and/or reduce the severity of cervical cancer or one or more symptoms thereof in a cervical cancer subject. In some embodiments, an nNOS inhibitor composition comprising L-NIO and/or a salt thereof, such as, for example, L-NIO dihydrochloride, may be administered to a cervical cancer subject to treat, ameliorate, and/or reduce the severity of cervical cancer or one or more symptoms thereof in the cervical cancer subject: (i) via a route selected from the group consisting of buccally, inhalation, intraarterially, intraarticularly, intracardially, intracerebroventricularly, intradermally, intramedullary, intramuscularly, intraperitoneally, intrathecally, intravenously, intraventricularly, nasally, ophthalmically, orally, rectally, sublabially, sublingually, subcutaneously, topically, and transdermally; and/or (ii) with a frequency selected from the group consisting of four times daily, three times daily, twice daily, once daily, once every two days, once every three days, once every four days, once every five days, once every six days, once a week, once every eight days, once every nine days, once every ten days, once every two weeks, once every three weeks, and once a month; and/or (iii) at a dosage (based on subject weight) selected from the group consisting of 0.1 μg/kg, 0.5 μg/kg, 1 μg/kg, 1.5 μg/kg, 2.0 μg/kg, 2.5 μg/kg, 3.0 μg/kg, 3.5 μg/kg, 4.0 μg/kg, 4.5 μg/kg, 5.0 μg/kg, 10 μg/kg, 15 μg/kg, 20 μg/kg, 25 μg/kg, 30 μg/kg, 35 μg/kg, 40 μg/kg, 45 μg/kg, 50 μg/kg, 55 μg/kg, 60 μg/kg, 65 μg/kg, 70 μg/kg, 75 μg/kg, 80 μg/kg, 85 μg/kg, 90 μg/kg, 95 μg/kg, 100 μg/kg, 150 μg/kg, 200 μg/kg, 250 μg/kg, 300 μg/kg, 350 μg/kg, 400 μg/kg, 450 μg/kg, 500 μg/kg, 550 μg/kg, 600 μg/kg, 650 μg/kg, 700 μg/kg, 750 μg/kg, 800 μg/kg, 850 μg/kg, 900 μg/kg, 1 mg/kg, 1.5 mg/kg, 2.0 mg/kg, 2.5 mg/kg, 3 mg/kg, 3.5 mg/kg, 4.0 mg/kg, 4.5 mg/kg, 5 mg/kg, 5.5 mg/kg, 6 mg/kg, 6.5 mg/kg, 7 mg/kg, 7.5 mg/kg, 8 mg/kg, 8.5 mg/kg, 9 mg/kg, 9.5 mg/kg, 10 mg/kg 10.5 mg/kg, 11 mg/kg, 11.5 mg/kg, 12 mg/kg, 12.5 mg/kg, 13 mg/kg, 13.5 mg/kg, 14 mg/kg, 14.5 mg/kg, 15 mg/kg, 16 mg/kg, 17 mg/kg, 18 mg/kg, 19 mg/kg, 20 mg/kg, 21 mg/kg, 22 mg/kg, 23 mg/kg, 24 mg/kg, 25 mg/kg, 30 mg/kg, 35 mg/kg, 40 mg/kg, 45 mg/kg, 50 mg/kg, 55 mg/kg, 60 mg/kg, 65 mg/kg, 70 mg/kg, 75 mg/kg, 80 mg/kg, 85 mg/kg, 90 mg/kg, 95 mg/kg, 100 mg/kg, 105 mg/kg, 110 mg/kg, 115 mg/kg, 120 mg/kg, 125 mg/kg, 130 mg/kg, 135 mg/kg, 140 mg/kg, 145 mg/kg, 150 mg/kg, 155 mg/kg, 160 mg/kg, 165 mg/kg, 170 mg/kg, 175 mg/kg, 180 mg/kg, 185 mg/kg, 190 mg/kg, 195 mg/kg, 200 mg/kg, and any fraction or integer in between any two of the preceding amounts.

In some embodiments, a NOS inhibitor composition comprising ARL-17477 and/or a salt thereof, such as, for example, ARL-17477 dihydrochloride, may be administered as a suitable dosage form, as described herein, to treat, ameliorate, and/or reduce the severity of cervical cancer or one or more symptoms thereof in a cervical cancer subject. In certain embodiments, an nNOS inhibitor composition comprising ARL-17477 and/or a salt thereof, such as, for example, ARL-17477 dihydrochloride, may be administered to a cervical cancer subject to treat, ameliorate, and/or reduce the severity of cervical cancer or one or more symptoms thereof in the cervical cancer subject: (i) via a route selected from the group consisting of buccally, inhalation, intraarterially, intraarticularly, intracardially, intracerebroventricularly, intradermally, intramedullary, intramuscularly, intraperitoneally, intrathecally, intravenously, intraventricularly, nasally, ophthalmically, orally, rectally, sublabially, sublingually, subcutaneously, topically, and transdermally; and/or (ii) with a frequency selected from the group consisting of four times daily, three times daily, twice daily, once daily, once every two days, once every three days, once every four days, once every five days, once every six days, once a week, once every eight days, once every nine days, once every ten days, once every two weeks, once every three weeks, and once a month; and/or (iii) at a dosage (based on subject weight) selected from the group consisting of 0.1 μg/kg, 0.5 μg/kg, 1 μg/kg, 1.5 μg/kg, 2.0 μg/kg, 2.5 μg/kg, 3.0 μg/kg, 3.5 μg/kg, 4.0 μg/kg, 4.5 μg/kg, 5.0 μg/kg, 10 μg/kg, 15 μg/kg, 20 μg/kg, 25 μg/kg, 30 μg/kg, 35 μg/kg, 40 μg/kg, 45 μg/kg, 50 μg/kg, 55 μg/kg, 60 μg/kg, 65 μg/kg, 70 μg/kg, 75 μg/kg, 80 μg/kg, 85 μg/kg, 90 μg/kg, 95 μg/kg, 100 μg/kg, 150 μg/kg, 200 μg/kg, 250 μg/kg, 300 μg/kg, 350 μg/kg, 400 μg/kg, 450 μg/kg, 500 μg/kg, 550 μg/kg, 600 μg/kg, 650 μg/kg, 700 μg/kg, 750 μg/kg, 800 μg/kg, 850 μg/kg, 900 μg/kg, 1 mg/kg, 1.5 mg/kg, 2.0 mg/kg, 2.5 mg/kg, 3 mg/kg, 3.5 mg/kg, 4.0 mg/kg, 4.5 mg/kg, 5 mg/kg, 5.5 mg/kg, 6 mg/kg, 6.5 mg/kg, 7 mg/kg, 7.5 mg/kg, 8 mg/kg, 8.5 mg/kg, 9 mg/kg, 9.5 mg/kg, 10 mg/kg 10.5 mg/kg, 11 mg/kg, 11.5 mg/kg, 12 mg/kg, 12.5 mg/kg, 13 mg/kg, 13.5 mg/kg, 14 mg/kg, 14.5 mg/kg, 15 mg/kg, 16 mg/kg, 17 mg/kg, 18 mg/kg, 19 mg/kg, 20 mg/kg, 21 mg/kg, 22 mg/kg, 23 mg/kg, 24 mg/kg, 25 mg/kg, 30 mg/kg, 35 mg/kg, 40 mg/kg, 45 mg/kg, 50 mg/kg, 55 mg/kg, 60 mg/kg, 65 mg/kg, 70 mg/kg, 75 mg/kg, 80 mg/kg, 85 mg/kg, 90 mg/kg, 95 mg/kg, 100 mg/kg, 105 mg/kg, 110 mg/kg, 115 mg/kg, 120 mg/kg, 125 mg/kg, 130 mg/kg, 135 mg/kg, 140 mg/kg, 145 mg/kg, 150 mg/kg, 155 mg/kg, 160 mg/kg, 165 mg/kg, 170 mg/kg, 175 mg/kg, 180 mg/kg, 185 mg/kg, 190 mg/kg, 195 mg/kg, 200 mg/kg, and any fraction or integer in between any two of the preceding amounts.

In certain embodiments, a NOS inhibitor composition comprising L-NNA and/or a salt thereof may be administered as a suitable dosage form, as described herein, to treat, ameliorate, and/or reduce the severity of cervical cancer or one or more symptoms thereof in a cervical cancer subject. In some embodiments, an nNOS inhibitor composition comprising L-NNA and/or a salt thereof may be administered to a cervical cancer subject to treat, ameliorate, and/or reduce the severity of cervical cancer or one or more symptoms thereof in the cervical cancer subject: (i) via a route selected from the group consisting of buccally, inhalation, intraarterially, intraarticularly, intracardially, intracerebroventricularly, intradermally, intramedullary, intramuscularly, intraperitoneally, intrathecally, intravenously, intraventricularly, nasally, ophthalmically, orally, rectally, sublabially, sublingually, subcutaneously, topically, and transdermally; and/or (ii) with a frequency selected from the group consisting of four times daily, three times daily, twice daily, once daily, once every two days, once every three days, once every four days, once every five days, once every six days, once a week, once every eight days, once every nine days, once every ten days, once every two weeks, once every three weeks, and once a month; and/or (iii) at a dosage (based on subject weight) selected from the group consisting of 0.1 μg/kg, 0.5 μg/kg, 1 μg/kg, 1.5 μg/kg, 2.0 μg/kg, 2.5 μg/kg, 3.0 μg/kg, 3.5 μg/kg, 4.0 μg/kg, 4.5 μg/kg, 5.0 μg/kg, 10 μg/kg, 15 μg/kg, 20 μg/kg, 25 μg/kg, 30 μg/kg, 35 μg/kg, 40 μg/kg, 45 μg/kg, 50 μg/kg, 55 μg/kg, 60 μg/kg, 65 μg/kg, 70 μg/kg, 75 μg/kg, 80 μg/kg, 85 μg/kg, 90 μg/kg, 95 μg/kg, 100 μg/kg, 150 μg/kg, 200 μg/kg, 250 μg/kg, 300 μg/kg, 350 μg/kg, 400 μg/kg, 450 μg/kg, 500 μg/kg, 550 μg/kg, 600 μg/kg, 650 μg/kg, 700 μg/kg, 750 μg/kg, 800 μg/kg, 850 μg/kg, 900 μg/kg, 1 mg/kg, 1.5 mg/kg, 2.0 mg/kg, 2.5 mg/kg, 3 mg/kg, 3.5 mg/kg, 4.0 mg/kg, 4.5 mg/kg, 5 mg/kg, 5.5 mg/kg, 6 mg/kg, 6.5 mg/kg, 7 mg/kg, 7.5 mg/kg, 8 mg/kg, 8.5 mg/kg, 9 mg/kg, 9.5 mg/kg, 10 mg/kg 10.5 mg/kg, 11 mg/kg, 11.5 mg/kg, 12 mg/kg, 12.5 mg/kg, 13 mg/kg, 13.5 mg/kg, 14 mg/kg, 14.5 mg/kg, 15 mg/kg, 16 mg/kg, 17 mg/kg, 18 mg/kg, 19 mg/kg, 20 mg/kg, 21 mg/kg, 22 mg/kg, 23 mg/kg, 24 mg/kg, 25 mg/kg, 30 mg/kg, 35 mg/kg, 40 mg/kg, 45 mg/kg, 50 mg/kg, 55 mg/kg, 60 mg/kg, 65 mg/kg, 70 mg/kg, 75 mg/kg, 80 mg/kg, 85 mg/kg, 90 mg/kg, 95 mg/kg, 100 mg/kg, 105 mg/kg, 110 mg/kg, 115 mg/kg, 120 mg/kg, 125 mg/kg, 130 mg/kg, 135 mg/kg, 140 mg/kg, 145 mg/kg, 150 mg/kg, 155 mg/kg, 160 mg/kg, 165 mg/kg, 170 mg/kg, 175 mg/kg, 180 mg/kg, 185 mg/kg, 190 mg/kg, 195 mg/kg, 200 mg/kg, and any fraction or integer in between any two of the preceding amounts.

In some embodiments, a NOS inhibitor composition comprising L-NAME and/or a salt thereof, such as, for example, L-NAME hydrochloride, may be administered as a suitable dosage form, as described herein, to treat, ameliorate, and/or reduce the severity of cervical cancer or one or more symptoms thereof in a cervical cancer subject. In certain embodiments, an nNOS inhibitor composition comprising L-NAME and/or a salt thereof, such as, for example, L-NAME hydrochloride, may be administered to a cervical cancer subject to treat, ameliorate, and/or reduce the severity of cervical cancer or one or more symptoms thereof in the cervical cancer subject: (i) via a route selected from the group consisting of buccally, inhalation, intraarterially, intraarticularly, intracardially, intracerebroventricularly, intradermally, intramedullary, intramuscularly, intraperitoneally, intrathecally, intravenously, intraventricularly, nasally, ophthalmically, orally, rectally, sublabially, sublingually, subcutaneously, topically, and transdermally; and/or (ii) with a frequency selected from the group consisting of four times daily, three times daily, twice daily, once daily, once every two days, once every three days, once every four days, once every five days, once every six days, once a week, once every eight days, once every nine days, once every ten days, once every two weeks, once every three weeks, and once a month; and/or (iii) at a dosage (based on subject weight) selected from the group consisting of 0.1 μg/kg, 0.5 μg/kg, 1 μg/kg, 1.5 μg/kg, 2.0 μg/kg, 2.5 μg/kg, 3.0 μg/kg, 3.5 μg/kg, 4.0 μg/kg, 4.5 μg/kg, 5.0 μg/kg, 10 μg/kg, 15 μg/kg, 20 μg/kg, 25 μg/kg, 30 μg/kg, 35 μg/kg, 40 μg/kg, 45 μg/kg, 50 μg/kg, 55 μg/kg, 60 μg/kg, 65 μg/kg, 70 μg/kg, 75 μg/kg, 80 μg/kg, 85 μg/kg, 90 μg/kg, 95 μg/kg, 100 μg/kg, 150 μg/kg, 200 μg/kg, 250 μg/kg, 300 μg/kg, 350 μg/kg, 400 μg/kg, 450 μg/kg, 500 μg/kg, 550 μg/kg, 600 μg/kg, 650 μg/kg, 700 μg/kg, 750 μg/kg, 800 μg/kg, 850 μg/kg, 900 μg/kg, 1 mg/kg, 1.5 mg/kg, 2.0 mg/kg, 2.5 mg/kg, 3 mg/kg, 3.5 mg/kg, 4.0 mg/kg, 4.5 mg/kg, 5 mg/kg, 5.5 mg/kg, 6 mg/kg, 6.5 mg/kg, 7 mg/kg, 7.5 mg/kg, 8 mg/kg, 8.5 mg/kg, 9 mg/kg, 9.5 mg/kg, 10 mg/kg 10.5 mg/kg, 11 mg/kg, 11.5 mg/kg, 12 mg/kg, 12.5 mg/kg, 13 mg/kg, 13.5 mg/kg, 14 mg/kg, 14.5 mg/kg, 15 mg/kg, 16 mg/kg, 17 mg/kg, 18 mg/kg, 19 mg/kg, 20 mg/kg, 21 mg/kg, 22 mg/kg, 23 mg/kg, 24 mg/kg, 25 mg/kg, 30 mg/kg, 35 mg/kg, 40 mg/kg, 45 mg/kg, 50 mg/kg, 55 mg/kg, 60 mg/kg, 65 mg/kg, 70 mg/kg, 75 mg/kg, 80 mg/kg, 85 mg/kg, 90 mg/kg, 95 mg/kg, 100 mg/kg, 105 mg/kg, 110 mg/kg, 115 mg/kg, 120 mg/kg, 125 mg/kg, 130 mg/kg, 135 mg/kg, 140 mg/kg, 145 mg/kg, 150 mg/kg, 155 mg/kg, 160 mg/kg, 165 mg/kg, 170 mg/kg, 175 mg/kg, 180 mg/kg, 185 mg/kg, 190 mg/kg, 195 mg/kg, 200 mg/kg, and any fraction or integer in between any two of the preceding amounts.

In certain embodiments, a NOS inhibitor composition comprising L-NMMA and/or a salt thereof, such as, for example, L-NMMA acetate, may be administered as a suitable dosage form, as described herein, to treat, ameliorate, and/or reduce the severity of cervical cancer or one or more symptoms thereof in a cervical cancer subject. In some embodiments, an nNOS inhibitor composition comprising L-NMMA and/or a salt thereof, such as, for example, L-NMMA acetate, may be administered to a cervical cancer subject to treat, ameliorate, and/or reduce the severity of cervical cancer or one or more symptoms thereof in the cervical cancer subject: (i) via a route selected from the group consisting of buccally, inhalation, intraarterially, intraarticularly, intracardially, intracerebroventricularly, intradermally, intramedullary, intramuscularly, intraperitoneally, intrathecally, intravenously, intraventricularly, nasally, ophthalmically, orally, rectally, sublabially, sublingually, subcutaneously, topically, and transdermally; and/or (ii) with a frequency selected from the group consisting of four times daily, three times daily, twice daily, once daily, once every two days, once every three days, once every four days, once every five days, once every six days, once a week, once every eight days, once every nine days, once every ten days, once every two weeks, once every three weeks, and once a month; and/or (iii) at a dosage (based on subject weight) selected from the group consisting of 0.1 μg/kg, 0.5 μg/kg, 1 μg/kg, 1.5 μg/kg, 2.0 μg/kg, 2.5 μg/kg, 3.0 μg/kg, 3.5 μg/kg, 4.0 μg/kg, 4.5 μg/kg, 5.0 μg/kg, 10 μg/kg, 15 μg/kg, 20 μg/kg, 25 μg/kg, 30 μg/kg, 35 μg/kg, 40 μg/kg, 45 μg/kg, 50 μg/kg, 55 μg/kg, 60 μg/kg, 65 μg/kg, 70 μg/kg, 75 μg/kg, 80 μg/kg, 85 μg/kg, 90 μg/kg, 95 μg/kg, 100 μg/kg, 150 μg/kg, 200 μg/kg, 250 μg/kg, 300 μg/kg, 350 μg/kg, 400 μg/kg, 450 μg/kg, 500 μg/kg, 550 μg/kg, 600 μg/kg, 650 μg/kg, 700 μg/kg, 750 μg/kg, 800 μg/kg, 850 μg/kg, 900 μg/kg, 1 mg/kg, 1.5 mg/kg, 2.0 mg/kg, 2.5 mg/kg, 3 mg/kg, 3.5 mg/kg, 4.0 mg/kg, 4.5 mg/kg, 5 mg/kg, 5.5 mg/kg, 6 mg/kg, 6.5 mg/kg, 7 mg/kg, 7.5 mg/kg, 8 mg/kg, 8.5 mg/kg, 9 mg/kg, 9.5 mg/kg, 10 mg/kg 10.5 mg/kg, 11 mg/kg, 11.5 mg/kg, 12 mg/kg, 12.5 mg/kg, 13 mg/kg, 13.5 mg/kg, 14 mg/kg, 14.5 mg/kg, 15 mg/kg, 16 mg/kg, 17 mg/kg, 18 mg/kg, 19 mg/kg, 20 mg/kg, 21 mg/kg, 22 mg/kg, 23 mg/kg, 24 mg/kg, 25 mg/kg, 30 mg/kg, 35 mg/kg, 40 mg/kg, 45 mg/kg, 50 mg/kg, 55 mg/kg, 60 mg/kg, 65 mg/kg, 70 mg/kg, 75 mg/kg, 80 mg/kg, 85 mg/kg, 90 mg/kg, 95 mg/kg, 100 mg/kg, 105 mg/kg, 110 mg/kg, 115 mg/kg, 120 mg/kg, 125 mg/kg, 130 mg/kg, 135 mg/kg, 140 mg/kg, 145 mg/kg, 150 mg/kg, 155 mg/kg, 160 mg/kg, 165 mg/kg, 170 mg/kg, 175 mg/kg, 180 mg/kg, 185 mg/kg, 190 mg/kg, 195 mg/kg, 200 mg/kg, and any fraction or integer in between any two of the preceding amounts.

In some embodiments, a NOS inhibitor composition comprising GW-274150 and/or a salt thereof, such as, for example, GW-274150 phosphate, may be administered as a suitable dosage form, as described herein, to treat, ameliorate, and/or reduce the severity of cervical cancer or one or more symptoms thereof in a cervical cancer subject. In certain embodiments, an nNOS inhibitor composition comprising GW-274150 and/or a salt thereof, such as, for example, GW-274150 phosphate, may be administered to a cervical cancer subject to treat, ameliorate, and/or reduce the severity of cervical cancer or one or more symptoms thereof in the cervical cancer subject: (i) via a route selected from the group consisting of buccally, inhalation, intraarterially, intraarticularly, intracardially, intracerebroventricularly, intradermally, intramedullary, intramuscularly, intraperitoneally, intrathecally, intravenously, intraventricularly, nasally, ophthalmically, orally, rectally, sublabially, sublingually, subcutaneously, topically, and transdermally; and/or (ii) with a frequency selected from the group consisting of four times daily, three times daily, twice daily, once daily, once every two days, once every three days, once every four days, once every five days, once every six days, once a week, once every eight days, once every nine days, once every ten days, once every two weeks, once every three weeks, and once a month; and/or (iii) at a dosage (based on subject weight) selected from the group consisting of 0.1 μg/kg, 0.5 μg/kg, 1 μg/kg, 1.5 μg/kg, 2.0 μg/kg, 2.5 μg/kg, 3.0 μg/kg, 3.5 μg/kg, 4.0 μg/kg, 4.5 μg/kg, 5.0 μg/kg, 10 μg/kg, 15 μg/kg, 20 μg/kg, 25 μg/kg, 30 μg/kg, 35 μg/kg, 40 μg/kg, 45 μg/kg, 50 μg/kg, 55 μg/kg, 60 μg/kg, 65 μg/kg, 70 μg/kg, 75 μg/kg, 80 μg/kg, 85 μg/kg, 90 μg/kg, 95 μg/kg, 100 μg/kg, 150 μg/kg, 200 μg/kg, 250 μg/kg, 300 μg/kg, 350 μg/kg, 400 μg/kg, 450 μg/kg, 500 μg/kg, 550 μg/kg, 600 μg/kg, 650 μg/kg, 700 μg/kg, 750 μg/kg, 800 μg/kg, 850 μg/kg, 900 μg/kg, 1 mg/kg, 1.5 mg/kg, 2.0 mg/kg, 2.5 mg/kg, 3 mg/kg, 3.5 mg/kg, 4.0 mg/kg, 4.5 mg/kg, 5 mg/kg, 5.5 mg/kg, 6 mg/kg, 6.5 mg/kg, 7 mg/kg, 7.5 mg/kg, 8 mg/kg, 8.5 mg/kg, 9 mg/kg, 9.5 mg/kg, 10 mg/kg 10.5 mg/kg, 11 mg/kg, 11.5 mg/kg, 12 mg/kg, 12.5 mg/kg, 13 mg/kg, 13.5 mg/kg, 14 mg/kg, 14.5 mg/kg, 15 mg/kg, 16 mg/kg, 17 mg/kg, 18 mg/kg, 19 mg/kg, 20 mg/kg, 21 mg/kg, 22 mg/kg, 23 mg/kg, 24 mg/kg, 25 mg/kg, 30 mg/kg, 35 mg/kg, 40 mg/kg, 45 mg/kg, 50 mg/kg, 55 mg/kg, 60 mg/kg, 65 mg/kg, 70 mg/kg, 75 mg/kg, 80 mg/kg, 85 mg/kg, 90 mg/kg, 95 mg/kg, 100 mg/kg, 105 mg/kg, 110 mg/kg, 115 mg/kg, 120 mg/kg, 125 mg/kg, 130 mg/kg, 135 mg/kg, 140 mg/kg, 145 mg/kg, 150 mg/kg, 155 mg/kg, 160 mg/kg, 165 mg/kg, 170 mg/kg, 175 mg/kg, 180 mg/kg, 185 mg/kg, 190 mg/kg, 195 mg/kg, 200 mg/kg, and any fraction or integer in between any two of the preceding amounts.

In certain embodiments, a NOS inhibitor composition comprising L-NIL and/or a salt thereof, such as, for example, L-NIL hydrochloride, may be administered as a suitable dosage form, as described herein, to treat, ameliorate, and/or reduce the severity of cervical cancer or one or more symptoms thereof in a cervical cancer subject. In some embodiments, an nNOS inhibitor composition comprising L-NIL and/or a salt thereof, such as, for example, L-NIL hydrochloride, may be administered to a cervical cancer subject to treat, ameliorate, and/or reduce the severity of cervical cancer or one or more symptoms thereof in the cervical cancer subject: (i) via a route selected from the group consisting of buccally, inhalation, intraarterially, intraarticularly, intracardially, intracerebroventricularly, intradermally, intramedullary, intramuscularly, intraperitoneally, intrathecally, intravenously, intraventricularly, nasally, ophthalmically, orally, rectally, sublabially, sublingually, subcutaneously, topically, and transdermally; and/or (ii) with a frequency selected from the group consisting of four times daily, three times daily, twice daily, once daily, once every two days, once every three days, once every four days, once every five days, once every six days, once a week, once every eight days, once every nine days, once every ten days, once every two weeks, once every three weeks, and once a month; and/or (iii) at a dosage (based on subject weight) selected from the group consisting of 0.1 μg/kg, 0.5 μg/kg, 1 μg/kg, 1.5 μg/kg, 2.0 μg/kg, 2.5 μg/kg, 3.0 μg/kg, 3.5 μg/kg, 4.0 μg/kg, 4.5 μg/kg, 5.0 μg/kg, 10 μg/kg, 15 μg/kg, 20 μg/kg, 25 μg/kg, 30 μg/kg, 35 μg/kg, 40 μg/kg, 45 μg/kg, 50 μg/kg, 55 μg/kg, 60 μg/kg, 65 μg/kg, 70 μg/kg, 75 μg/kg, 80 μg/kg, 85 μg/kg, 90 μg/kg, 95 μg/kg, 100 μg/kg, 150 μg/kg, 200 μg/kg, 250 μg/kg, 300 μg/kg, 350 μg/kg, 400 μg/kg, 450 μg/kg, 500 μg/kg, 550 μg/kg, 600 μg/kg, 650 μg/kg, 700 μg/kg, 750 μg/kg, 800 μg/kg, 850 μg/kg, 900 μg/kg, 1 mg/kg, 1.5 mg/kg, 2.0 mg/kg, 2.5 mg/kg, 3 mg/kg, 3.5 mg/kg, 4.0 mg/kg, 4.5 mg/kg, 5 mg/kg, 5.5 mg/kg, 6 mg/kg, 6.5 mg/kg, 7 mg/kg, 7.5 mg/kg, 8 mg/kg, 8.5 mg/kg, 9 mg/kg, 9.5 mg/kg, 10 mg/kg 10.5 mg/kg, 11 mg/kg, 11.5 mg/kg, 12 mg/kg, 12.5 mg/kg, 13 mg/kg, 13.5 mg/kg, 14 mg/kg, 14.5 mg/kg, 15 mg/kg, 16 mg/kg, 17 mg/kg, 18 mg/kg, 19 mg/kg, 20 mg/kg, 21 mg/kg, 22 mg/kg, 23 mg/kg, 24 mg/kg, 25 mg/kg, 30 mg/kg, 35 mg/kg, 40 mg/kg, 45 mg/kg, 50 mg/kg, 55 mg/kg, 60 mg/kg, 65 mg/kg, 70 mg/kg, 75 mg/kg, 80 mg/kg, 85 mg/kg, 90 mg/kg, 95 mg/kg, 100 mg/kg, 105 mg/kg, 110 mg/kg, 115 mg/kg, 120 mg/kg, 125 mg/kg, 130 mg/kg, 135 mg/kg, 140 mg/kg, 145 mg/kg, 150 mg/kg, 155 mg/kg, 160 mg/kg, 165 mg/kg, 170 mg/kg, 175 mg/kg, 180 mg/kg, 185 mg/kg, 190 mg/kg, 195 mg/kg, 200 mg/kg, and any fraction or integer in between any two of the preceding amounts.

In some embodiments, a NOS inhibitor composition comprising 1400W and/or a salt thereof, such as, for example, 1400W dihydrochloride, may be administered as a suitable dosage form, as described herein, to treat, ameliorate, and/or reduce the severity of cervical cancer or one or more symptoms thereof in a cervical cancer subject. In certain embodiments, an nNOS inhibitor composition comprising 1400W and/or a salt thereof, such as, for example, 1400W dihydrochloride, may be administered to a cervical cancer subject to treat, ameliorate, and/or reduce the severity of cervical cancer or one or more symptoms thereof in the cervical cancer subject: (i) via a route selected from the group consisting of buccally, inhalation, intraarterially, intraarticularly, intracardially, intracerebroventricularly, intradermally, intramedullary, intramuscularly, intraperitoneally, intrathecally, intravenously, intraventricularly, nasally, ophthalmically, orally, rectally, sublabially, sublingually, subcutaneously, topically, and transdermally; and/or (ii) with a frequency selected from the group consisting of four times daily, three times daily, twice daily, once daily, once every two days, once every three days, once every four days, once every five days, once every six days, once a week, once every eight days, once every nine days, once every ten days, once every two weeks, once every three weeks, and once a month; and/or (iii) at a dosage (based on subject weight) selected from the group consisting of 0.1 μg/kg, 0.5 μg/kg, 1 μg/kg, 1.5 μg/kg, 2.0 μg/kg, 2.5 μg/kg, 3.0 μg/kg, 3.5 μg/kg, 4.0 μg/kg, 4.5 μg/kg, 5.0 μg/kg, 10 μg/kg, 15 μg/kg, 20 μg/kg, 25 μg/kg, 30 μg/kg, 35 μg/kg, 40 μg/kg, 45 μg/kg, 50 μg/kg, 55 μg/kg, 60 μg/kg, 65 μg/kg, 70 μg/kg, 75 μg/kg, 80 μg/kg, 85 μg/kg, 90 μg/kg, 95 μg/kg, 100 μg/kg, 150 μg/kg, 200 μg/kg, 250 μg/kg, 300 μg/kg, 350 μg/kg, 400 μg/kg, 450 μg/kg, 500 μg/kg, 550 μg/kg, 600 μg/kg, 650 μg/kg, 700 μg/kg, 750 μg/kg, 800 μg/kg, 850 μg/kg, 900 μg/kg, 1 mg/kg, 1.5 mg/kg, 2.0 mg/kg, 2.5 mg/kg, 3 mg/kg, 3.5 mg/kg, 4.0 mg/kg, 4.5 mg/kg, 5 mg/kg, 5.5 mg/kg, 6 mg/kg, 6.5 mg/kg, 7 mg/kg, 7.5 mg/kg, 8 mg/kg, 8.5 mg/kg, 9 mg/kg, 9.5 mg/kg, 10 mg/kg 10.5 mg/kg, 11 mg/kg, 11.5 mg/kg, 12 mg/kg, 12.5 mg/kg, 13 mg/kg, 13.5 mg/kg, 14 mg/kg, 14.5 mg/kg, 15 mg/kg, 16 mg/kg, 17 mg/kg, 18 mg/kg, 19 mg/kg, 20 mg/kg, 21 mg/kg, 22 mg/kg, 23 mg/kg, 24 mg/kg, 25 mg/kg, 30 mg/kg, 35 mg/kg, 40 mg/kg, 45 mg/kg, 50 mg/kg, 55 mg/kg, 60 mg/kg, 65 mg/kg, 70 mg/kg, 75 mg/kg, 80 mg/kg, 85 mg/kg, 90 mg/kg, 95 mg/kg, 100 mg/kg, 105 mg/kg, 110 mg/kg, 115 mg/kg, 120 mg/kg, 125 mg/kg, 130 mg/kg, 135 mg/kg, 140 mg/kg, 145 mg/kg, 150 mg/kg, 155 mg/kg, 160 mg/kg, 165 mg/kg, 170 mg/kg, 175 mg/kg, 180 mg/kg, 185 mg/kg, 190 mg/kg, 195 mg/kg, 200 mg/kg, and any fraction or integer in between any two of the preceding amounts.

In certain embodiments, a NOS inhibitor composition comprising OKN-007 and/or a salt thereof, such as, for example, OKN-007 sodium, may be administered as a suitable dosage form, as described herein, to treat, ameliorate, and/or reduce the severity of cervical cancer or one or more symptoms thereof in a cervical cancer subject. In some embodiments, an nNOS inhibitor composition comprising OKN-007 and/or a salt thereof, such as, for example, OKN-007 sodium, may be administered to a cervical cancer subject to treat, ameliorate, and/or reduce the severity of cervical cancer or one or more symptoms thereof in the cervical cancer subject: (i) via a route selected from the group consisting of buccally, inhalation, intraarterially, intraarticularly, intracardially, intracerebroventricularly, intradermally, intramedullary, intramuscularly, intraperitoneally, intrathecally, intravenously, intraventricularly, nasally, ophthalmically, orally, rectally, sublabially, sublingually, subcutaneously, topically, and transdermally; and/or (ii) with a frequency selected from the group consisting of four times daily, three times daily, twice daily, once daily, once every two days, once every three days, once every four days, once every five days, once every six days, once a week, once every eight days, once every nine days, once every ten days, once every two weeks, once every three weeks, and once a month; and/or (iii) at a dosage (based on subject weight) selected from the group consisting of 0.1 μg/kg, 0.5 μg/kg, 1 μg/kg, 1.5 μg/kg, 2.0 μg/kg, 2.5 μg/kg, 3.0 μg/kg, 3.5 μg/kg, 4.0 μg/kg, 4.5 μg/kg, 5.0 μg/kg, 10 μg/kg, 15 μg/kg, 20 μg/kg, 25 μg/kg, 30 μg/kg, 35 μg/kg, 40 μg/kg, 45 μg/kg, 50 μg/kg, 55 μg/kg, 60 μg/kg, 65 μg/kg, 70 μg/kg, 75 μg/kg, 80 μg/kg, 85 μg/kg, 90 μg/kg, 95 μg/kg, 100 μg/kg, 150 μg/kg, 200 μg/kg, 250 μg/kg, 300 μg/kg, 350 μg/kg, 400 μg/kg, 450 μg/kg, 500 μg/kg, 550 μg/kg, 600 μg/kg, 650 μg/kg, 700 μg/kg, 750 μg/kg, 800 μg/kg, 850 μg/kg, 900 μg/kg, 1 mg/kg, 1.5 mg/kg, 2.0 mg/kg, 2.5 mg/kg, 3 mg/kg, 3.5 mg/kg, 4.0 mg/kg, 4.5 mg/kg, 5 mg/kg, 5.5 mg/kg, 6 mg/kg, 6.5 mg/kg, 7 mg/kg, 7.5 mg/kg, 8 mg/kg, 8.5 mg/kg, 9 mg/kg, 9.5 mg/kg, 10 mg/kg 10.5 mg/kg, 11 mg/kg, 11.5 mg/kg, 12 mg/kg, 12.5 mg/kg, 13 mg/kg, 13.5 mg/kg, 14 mg/kg, 14.5 mg/kg, 15 mg/kg, 16 mg/kg, 17 mg/kg, 18 mg/kg, 19 mg/kg, 20 mg/kg, 21 mg/kg, 22 mg/kg, 23 mg/kg, 24 mg/kg, 25 mg/kg, 30 mg/kg, 35 mg/kg, 40 mg/kg, 45 mg/kg, 50 mg/kg, 55 mg/kg, 60 mg/kg, 65 mg/kg, 70 mg/kg, 75 mg/kg, 80 mg/kg, 85 mg/kg, 90 mg/kg, 95 mg/kg, 100 mg/kg, 105 mg/kg, 110 mg/kg, 115 mg/kg, 120 mg/kg, 125 mg/kg, 130 mg/kg, 135 mg/kg, 140 mg/kg, 145 mg/kg, 150 mg/kg, 155 mg/kg, 160 mg/kg, 165 mg/kg, 170 mg/kg, 175 mg/kg, 180 mg/kg, 185 mg/kg, 190 mg/kg, 195 mg/kg, 200 mg/kg, and any fraction or integer in between any two of the preceding amounts.

In some embodiments, a NOS inhibitor composition comprising BYK-191023 and/or a salt thereof, such as, for example, BYK-191023 dihydrochloride, may be administered as a suitable dosage form, as described herein, to treat, ameliorate, and/or reduce the severity of cervical cancer or one or more symptoms thereof in a cervical cancer subject. In certain embodiments, an nNOS inhibitor composition comprising BYK-191023 and/or a salt thereof, such as, for example, BYK-191023 dihydrochloride, may be administered to a cervical cancer subject to treat, ameliorate, and/or reduce the severity of cervical cancer or one or more symptoms thereof in the cervical cancer subject: (i) via a route selected from the group consisting of buccally, inhalation, intraarterially, intraarticularly, intracardially, intracerebroventricularly, intradermally, intramedullary, intramuscularly, intraperitoneally, intrathecally, intravenously, intraventricularly, nasally, ophthalmically, orally, rectally, sublabially, sublingually, subcutaneously, topically, and transdermally; and/or (ii) with a frequency selected from the group consisting of four times daily, three times daily, twice daily, once daily, once every two days, once every three days, once every four days, once every five days, once every six days, once a week, once every eight days, once every nine days, once every ten days, once every two weeks, once every three weeks, and once a month; and/or (iii) at a dosage (based on subject weight) selected from the group consisting of 0.1 μg/kg, 0.5 μg/kg, 1 μg/kg, 1.5 μg/kg, 2.0 μg/kg, 2.5 μg/kg, 3.0 μg/kg, 3.5 μg/kg, 4.0 μg/kg, 4.5 μg/kg, 5.0 μg/kg, 10 μg/kg, 15 μg/kg, 20 μg/kg, 25 μg/kg, 30 μg/kg, 35 μg/kg, 40 μg/kg, 45 μg/kg, 50 μg/kg, 55 μg/kg, 60 μg/kg, 65 μg/kg, 70 μg/kg, 75 μg/kg, 80 μg/kg, 85 μg/kg, 90 μg/kg, 95 μg/kg, 100 μg/kg, 150 μg/kg, 200 μg/kg, 250 μg/kg, 300 μg/kg, 350 μg/kg, 400 μg/kg, 450 μg/kg, 500 μg/kg, 550 μg/kg, 600 μg/kg, 650 μg/kg, 700 μg/kg, 750 μg/kg, 800 μg/kg, 850 μg/kg, 900 μg/kg, 1 mg/kg, 1.5 mg/kg, 2.0 mg/kg, 2.5 mg/kg, 3 mg/kg, 3.5 mg/kg, 4.0 mg/kg, 4.5 mg/kg, 5 mg/kg, 5.5 mg/kg, 6 mg/kg, 6.5 mg/kg, 7 mg/kg, 7.5 mg/kg, 8 mg/kg, 8.5 mg/kg, 9 mg/kg, 9.5 mg/kg, 10 mg/kg 10.5 mg/kg, 11 mg/kg, 11.5 mg/kg, 12 mg/kg, 12.5 mg/kg, 13 mg/kg, 13.5 mg/kg, 14 mg/kg, 14.5 mg/kg, 15 mg/kg, 16 mg/kg, 17 mg/kg, 18 mg/kg, 19 mg/kg, 20 mg/kg, 21 mg/kg, 22 mg/kg, 23 mg/kg, 24 mg/kg, 25 mg/kg, 30 mg/kg, 35 mg/kg, 40 mg/kg, 45 mg/kg, 50 mg/kg, 55 mg/kg, 60 mg/kg, 65 mg/kg, 70 mg/kg, 75 mg/kg, 80 mg/kg, 85 mg/kg, 90 mg/kg, 95 mg/kg, 100 mg/kg, 105 mg/kg, 110 mg/kg, 115 mg/kg, 120 mg/kg, 125 mg/kg, 130 mg/kg, 135 mg/kg, 140 mg/kg, 145 mg/kg, 150 mg/kg, 155 mg/kg, 160 mg/kg, 165 mg/kg, 170 mg/kg, 175 mg/kg, 180 mg/kg, 185 mg/kg, 190 mg/kg, 195 mg/kg, 200 mg/kg, and any fraction or integer in between any two of the preceding amounts.

In certain embodiments, a NOS inhibitor composition comprising CM544 and/or a salt thereof, such as, for example, CM544 dihydrochloride, may be administered as a suitable dosage form, as described herein, to treat, ameliorate, and/or reduce the severity of cervical cancer or one or more symptoms thereof in a cervical cancer subject. In some embodiments, an nNOS inhibitor composition comprising CM544 and/or a salt thereof, such as, for example, CM544 dihydrochloride, may be administered to a cervical cancer subject to treat, ameliorate, and/or reduce the severity of cervical cancer or one or more symptoms thereof in the cervical cancer subject: (i) via a route selected from the group consisting of buccally, inhalation, intraarterially, intraarticularly, intracardially, intracerebroventricularly, intradermally, intramedullary, intramuscularly, intraperitoneally, intrathecally, intravenously, intraventricularly, nasally, ophthalmically, orally, rectally, sublabially, sublingually, subcutaneously, topically, and transdermally; and/or (ii) with a frequency selected from the group consisting of four times daily, three times daily, twice daily, once daily, once every two days, once every three days, once every four days, once every five days, once every six days, once a week, once every eight days, once every nine days, once every ten days, once every two weeks, once every three weeks, and once a month; and/or (iii) at a dosage (based on subject weight) selected from the group consisting of 0.1 μg/kg, 0.5 μg/kg, 1 μg/kg, 1.5 μg/kg, 2.0 μg/kg, 2.5 μg/kg, 3.0 μg/kg, 3.5 μg/kg, 4.0 μg/kg, 4.5 μg/kg, 5.0 μg/kg, 10 μg/kg, 15 μg/kg, 20 μg/kg, 25 μg/kg, 30 μg/kg, 35 μg/kg, 40 μg/kg, 45 μg/kg, 50 μg/kg, 55 μg/kg, 60 μg/kg, 65 μg/kg, 70 μg/kg, 75 μg/kg, 80 μg/kg, 85 μg/kg, 90 μg/kg, 95 μg/kg, 100 μg/kg, 150 μg/kg, 200 μg/kg, 250 μg/kg, 300 μg/kg, 350 μg/kg, 400 μg/kg, 450 μg/kg, 500 μg/kg, 550 μg/kg, 600 μg/kg, 650 μg/kg, 700 μg/kg, 750 μg/kg, 800 μg/kg, 850 μg/kg, 900 μg/kg, 1 mg/kg, 1.5 mg/kg, 2.0 mg/kg, 2.5 mg/kg, 3 mg/kg, 3.5 mg/kg, 4.0 mg/kg, 4.5 mg/kg, 5 mg/kg, 5.5 mg/kg, 6 mg/kg, 6.5 mg/kg, 7 mg/kg, 7.5 mg/kg, 8 mg/kg, 8.5 mg/kg, 9 mg/kg, 9.5 mg/kg, 10 mg/kg 10.5 mg/kg, 11 mg/kg, 11.5 mg/kg, 12 mg/kg, 12.5 mg/kg, 13 mg/kg, 13.5 mg/kg, 14 mg/kg, 14.5 mg/kg, 15 mg/kg, 16 mg/kg, 17 mg/kg, 18 mg/kg, 19 mg/kg, 20 mg/kg, 21 mg/kg, 22 mg/kg, 23 mg/kg, 24 mg/kg, 25 mg/kg, 30 mg/kg, 35 mg/kg, 40 mg/kg, 45 mg/kg, 50 mg/kg, 55 mg/kg, 60 mg/kg, 65 mg/kg, 70 mg/kg, 75 mg/kg, 80 mg/kg, 85 mg/kg, 90 mg/kg, 95 mg/kg, 100 mg/kg, 105 mg/kg, 110 mg/kg, 115 mg/kg, 120 mg/kg, 125 mg/kg, 130 mg/kg, 135 mg/kg, 140 mg/kg, 145 mg/kg, 150 mg/kg, 155 mg/kg, 160 mg/kg, 165 mg/kg, 170 mg/kg, 175 mg/kg, 180 mg/kg, 185 mg/kg, 190 mg/kg, 195 mg/kg, 200 mg/kg, and any fraction or integer in between any two of the preceding amounts.

In certain embodiments, an nNOS inhibitor composition comprising one or more dsRNAs each comprising at least 30 base pairs, may be administered as a suitable dosage form, as described herein, to treat, ameliorate, and/or reduce the severity of cervical cancer or one or more symptoms thereof in a cervical cancer subject. In some embodiments, an nNOS inhibitor composition comprising one or more dsRNAs, each comprising at least 30 base pairs, may be administered to a cervical cancer subject to treat, ameliorate, and/or reduce the severity of cervical cancer or one or more symptoms thereof in the cervical cancer subject: (i) via a route selected from the group consisting of buccally, inhalation, intraarterially, intraarticularly, intracardially, intracerebroventricularly, intradermally, intramedullary, intramuscularly, intraperitoneally, intrathecally, intravenously, intraventricularly, nasally, ophthalmically, orally, rectally, sublabially, sublingually, subcutaneously, topically, and transdermally; and/or (ii) with a frequency selected from the group consisting of four times daily, three times daily, twice daily, once daily, once every two days, once every three days, once every four days, once every five days, once every six days, once a week, once every eight days, once every nine days, once every ten days, once every two weeks, once every three weeks, and once a month; and/or (iii) at a dosage (based on subject weight) selected from the group consisting of 0.1 μg/kg, 0.5 μg/kg, 1 μg/kg, 1.5 μg/kg, 2.0 μg/kg, 2.5 μg/kg, 3.0 μg/kg, 3.5 μg/kg, 4.0 μg/kg, 4.5 μg/kg, 5.0 μg/kg, 10 μg/kg, 15 μg/kg, 20 μg/kg, 25 μg/kg, 30 μg/kg, 35 μg/kg, 40 μg/kg, 45 μg/kg, 50 μg/kg, 55 μg/kg, 60 μg/kg, 65 μg/kg, 70 μg/kg, 75 μg/kg, 80 μg/kg, 85 μg/kg, 90 μg/kg, 95 μg/kg, 100 μg/kg, 150 μg/kg, 200 μg/kg, 250 μg/kg, 300 μg/kg, 350 μg/kg, 400 μg/kg, 450 μg/kg, 500 μg/kg, 550 μg/kg, 600 μg/kg, 650 μg/kg, 700 μg/kg, 750 μg/kg, 800 μg/kg, 850 μg/kg, 900 μg/kg, 1 mg/kg, 1.5 mg/kg, 2.0 mg/kg, 2.5 mg/kg, 3 mg/kg, 3.5 mg/kg, 4.0 mg/kg, 4.5 mg/kg, 5 mg/kg, 5.5 mg/kg, 6 mg/kg, 6.5 mg/kg, 7 mg/kg, 7.5 mg/kg, 8 mg/kg, 8.5 mg/kg, 9 mg/kg, 9.5 mg/kg, 10 mg/kg 10.5 mg/kg, 11 mg/kg, 11.5 mg/kg, 12 mg/kg, 12.5 mg/kg, 13 mg/kg, 13.5 mg/kg, 14 mg/kg, 14.5 mg/kg, 15 mg/kg, 16 mg/kg, 17 mg/kg, 18 mg/kg, 19 mg/kg, 20 mg/kg, 21 mg/kg, 22 mg/kg, 23 mg/kg, 24 mg/kg, 25 mg/kg, 30 mg/kg, 35 mg/kg, 40 mg/kg, 45 mg/kg, 50 mg/kg, 55 mg/kg, 60 mg/kg, 65 mg/kg, 70 mg/kg, 75 mg/kg, 80 mg/kg, 85 mg/kg, 90 mg/kg, 95 mg/kg, 100 mg/kg, 105 mg/kg, 110 mg/kg, 115 mg/kg, 120 mg/kg, 125 mg/kg, 130 mg/kg, 135 mg/kg, 140 mg/kg, 145 mg/kg, 150 mg/kg, 155 mg/kg, 160 mg/kg, 165 mg/kg, 170 mg/kg, 175 mg/kg, 180 mg/kg, 185 mg/kg, 190 mg/kg, 195 mg/kg, 200 mg/kg, and any fraction or integer in between any two of the preceding amounts.

In some embodiments, an nNOS inhibitor composition comprising one or more siRNAs, may be administered as a suitable dosage form, as described herein, to treat, ameliorate, and/or reduce the severity of cervical cancer or one or more symptoms thereof in a cervical cancer subject. In certain embodiments, an nNOS inhibitor composition comprising one or more siRNAs selected from the group consisting of SEQ ID NO. 4, SEQ ID NO. 5, SEQ ID NO. 6, SEQ ID NO. 7, SEQ ID NO. 8, and any combination thereof, may be administered as a suitable dosage form, as described herein, to treat, ameliorate, and/or reduce the severity of cervical cancer or one or more symptoms thereof in a cervical cancer subject. In some embodiments, an nNOS inhibitor composition comprising one or more siRNAs selected from the group consisting of SEQ ID NO. 4, SEQ ID NO. 5, SEQ ID NO. 6, SEQ ID NO. 7, SEQ ID NO. 8, and any combination thereof, may be administered to a cervical cancer subject to treat, ameliorate, and/or reduce the severity of cervical cancer or one or more symptoms thereof in the cervical cancer subject: (i) via a route selected from the group consisting of buccally, inhalation, intraarterially, intraarticularly, intracardially, intracerebroventricularly, intradermally, intramedullary, intramuscularly, intraperitoneally, intrathecally, intravenously, intraventricularly, nasally, ophthalmically, orally, rectally, sublabially, sublingually, subcutaneously, topically, and transdermally; and/or (ii) with a frequency selected from the group consisting of four times daily, three times daily, twice daily, once daily, once every two days, once every three days, once every four days, once every five days, once every six days, once a week, once every eight days, once every nine days, once every ten days, once every two weeks, once every three weeks, and once a month; and/or (iii) at a dosage (based on subject weight) selected from the group consisting of 0.1 μg/kg, 0.5 μg/kg, 1 μg/kg, 1.5 μg/kg, 2.0 μg/kg, 2.5 μg/kg, 3.0 μg/kg, 3.5 μg/kg, 4.0 μg/kg, 4.5 μg/kg, 5.0 μg/kg, 10 μg/kg, 15 μg/kg, 20 μg/kg, 25 μg/kg, 30 μg/kg, 35 μg/kg, 40 μg/kg, 45 μg/kg, 50 μg/kg, 55 μg/kg, 60 μg/kg, 65 μg/kg, 70 μg/kg, 75 μg/kg, 80 μg/kg, 85 μg/kg, 90 μg/kg, 95 μg/kg, 100 μg/kg, 150 μg/kg, 200 μg/kg, 250 μg/kg, 300 μg/kg, 350 μg/kg, 400 μg/kg, 450 μg/kg, 500 μg/kg, 550 μg/kg, 600 μg/kg, 650 μg/kg, 700 μg/kg, 750 μg/kg, 800 μg/kg, 850 μg/kg, 900 μg/kg, 1 mg/kg, 1.5 mg/kg, 2.0 mg/kg, 2.5 mg/kg, 3 mg/kg, 3.5 mg/kg, 4.0 mg/kg, 4.5 mg/kg, 5 mg/kg, 5.5 mg/kg, 6 mg/kg, 6.5 mg/kg, 7 mg/kg, 7.5 mg/kg, 8 mg/kg, 8.5 mg/kg, 9 mg/kg, 9.5 mg/kg, 10 mg/kg 10.5 mg/kg, 11 mg/kg, 11.5 mg/kg, 12 mg/kg, 12.5 mg/kg, 13 mg/kg, 13.5 mg/kg, 14 mg/kg, 14.5 mg/kg, 15 mg/kg, 16 mg/kg, 17 mg/kg, 18 mg/kg, 19 mg/kg, 20 mg/kg, 21 mg/kg, 22 mg/kg, 23 mg/kg, 24 mg/kg, 25 mg/kg, 30 mg/kg, 35 mg/kg, 40 mg/kg, 45 mg/kg, 50 mg/kg, 55 mg/kg, 60 mg/kg, 65 mg/kg, 70 mg/kg, 75 mg/kg, 80 mg/kg, 85 mg/kg, 90 mg/kg, 95 mg/kg, 100 mg/kg, 105 mg/kg, 110 mg/kg, 115 mg/kg, 120 mg/kg, 125 mg/kg, 130 mg/kg, 135 mg/kg, 140 mg/kg, 145 mg/kg, 150 mg/kg, 155 mg/kg, 160 mg/kg, 165 mg/kg, 170 mg/kg, 175 mg/kg, 180 mg/kg, 185 mg/kg, 190 mg/kg, 195 mg/kg, 200 mg/kg, and any fraction or integer in between any two of the preceding amounts.

In some embodiments, an nNOS inhibitor composition comprising one or more shRNAs, may be administered as a suitable dosage form, as described herein, to treat, ameliorate, and/or reduce the severity of cervical cancer or one or more symptoms thereof in a cervical cancer subject. In certain embodiments, an nNOS inhibitor composition comprising one or more shRNAs selected from the group consisting of SEQ ID NO. 9, SEQ ID NO. 10, SEQ ID NO. 11, SEQ ID NO. 12, SEQ ID NO. 13, SEQ ID NO. 14, and any combination thereof, may be administered as a suitable dosage form, as described herein, to treat, ameliorate, and/or reduce the severity of cervical cancer or one or more symptoms thereof in a cervical cancer subject. In some embodiments, an nNOS inhibitor composition comprising one or more shRNAs selected from the group consisting of SEQ ID NO. 9, SEQ ID NO. 10, SEQ ID NO. 11, SEQ ID NO. 12, SEQ ID NO. 13, SEQ ID NO. 14, and any combination thereof, may be administered to a cervical cancer subject to treat, ameliorate, and/or reduce the severity of cervical cancer or one or more symptoms thereof in the cervical cancer subject: (i) via a route selected from the group consisting of buccally, inhalation, intraarterially, intraarticularly, intracardially, intracerebroventricularly, intradermally, intramedullary, intramuscularly, intraperitoneally, intrathecally, intravenously, intraventricularly, nasally, ophthalmically, orally, rectally, sublabially, sublingually, subcutaneously, topically, and transdermally; and/or (ii) with a frequency selected from the group consisting of four times daily, three times daily, twice daily, once daily, once every two days, once every three days, once every four days, once every five days, once every six days, once a week, once every eight days, once every nine days, once every ten days, once every two weeks, once every three weeks, and once a month; and/or (iii) at a dosage (based on subject weight) selected from the group consisting of 0.1 μg/kg, 0.5 μg/kg, 1 μg/kg, 1.5 μg/kg, 2.0 μg/kg, 2.5 μg/kg, 3.0 μg/kg, 3.5 μg/kg, 4.0 μg/kg, 4.5 μg/kg, 5.0 μg/kg, 10 μg/kg, 15 μg/kg, 20 μg/kg, 25 μg/kg, 30 μg/kg, 35 μg/kg, 40 μg/kg, 45 μg/kg, 50 μg/kg, 55 μg/kg, 60 μg/kg, 65 μg/kg, 70 μg/kg, 75 μg/kg, 80 μg/kg, 85 μg/kg, 90 μg/kg, 95 μg/kg, 100 μg/kg, 150 μg/kg, 200 μg/kg, 250 μg/kg, 300 μg/kg, 350 μg/kg, 400 μg/kg, 450 μg/kg, 500 μg/kg, 550 μg/kg, 600 μg/kg, 650 μg/kg, 700 μg/kg, 750 μg/kg, 800 μg/kg, 850 μg/kg, 900 μg/kg, 1 mg/kg, 1.5 mg/kg, 2.0 mg/kg, 2.5 mg/kg, 3 mg/kg, 3.5 mg/kg, 4.0 mg/kg, 4.5 mg/kg, 5 mg/kg, 5.5 mg/kg, 6 mg/kg, 6.5 mg/kg, 7 mg/kg, 7.5 mg/kg, 8 mg/kg, 8.5 mg/kg, 9 mg/kg, 9.5 mg/kg, 10 mg/kg 10.5 mg/kg, 11 mg/kg, 11.5 mg/kg, 12 mg/kg, 12.5 mg/kg, 13 mg/kg, 13.5 mg/kg, 14 mg/kg, 14.5 mg/kg, 15 mg/kg, 16 mg/kg, 17 mg/kg, 18 mg/kg, 19 mg/kg, 20 mg/kg, 21 mg/kg, 22 mg/kg, 23 mg/kg, 24 mg/kg, 25 mg/kg, 30 mg/kg, 35 mg/kg, 40 mg/kg, 45 mg/kg, 50 mg/kg, 55 mg/kg, 60 mg/kg, 65 mg/kg, 70 mg/kg, 75 mg/kg, 80 mg/kg, 85 mg/kg, 90 mg/kg, 95 mg/kg, 100 mg/kg, 105 mg/kg, 110 mg/kg, 115 mg/kg, 120 mg/kg, 125 mg/kg, 130 mg/kg, 135 mg/kg, 140 mg/kg, 145 mg/kg, 150 mg/kg, 155 mg/kg, 160 mg/kg, 165 mg/kg, 170 mg/kg, 175 mg/kg, 180 mg/kg, 185 mg/kg, 190 mg/kg, 195 mg/kg, 200 mg/kg, and any fraction or integer in between any two of the preceding amounts.

In some embodiments, an nNOS inhibitor composition comprising one or more miRNAs, may be administered as a suitable dosage form, as described herein, to treat, ameliorate, and/or reduce the severity of cervical cancer or one or more symptoms thereof in a cervical cancer subject. In certain embodiments, an nNOS inhibitor composition comprising one or more miRNAs selected from the group consisting of miR-146a, hsa-miR-146a-5p, hsa-miR-1273f, hsa-miR-143-3p, hsa-miR-147a, hsa-miR-3622a-5p, hsa-miR-3911, hsa-miR-4269, hsa-miR-4708-5p, hsa-miR-4710, hsa-miR-4770, hsa-miR-4792, hsa-miR-574-5p, hsa-miR-6088, hsa-miR-644a, hsa-miR-6715b-5p, hsa-miR-6768-5p, hsa-miR-6867-5p, hsa-miR-7855-5p, hsa-miR-8485, and any combination thereof, may be administered as a suitable dosage form, as described herein, to treat, ameliorate, and/or reduce the severity of cervical cancer or one or more symptoms thereof in a cervical cancer subject. In some embodiments, an nNOS inhibitor composition comprising one or more miRNAs selected from the group consisting of miR-146a, hsa-miR-146a-5p, hsa-miR-1273f, hsa-miR-143-3p, hsa-miR-147a, hsa-miR-3622a-5p, hsa-miR-3911, hsa-miR-4269, hsa-miR-4708-5p, hsa-miR-4710, hsa-miR-4770, hsa-miR-4792, hsa-miR-574-5p, hsa-miR-6088, hsa-miR-644a, hsa-miR-6715b-5p, hsa-miR-6768-5p, hsa-miR-6867-5p, hsa-miR-7855-5p, hsa-miR-8485, and any combination thereof, may be administered to a cervical cancer subject to treat, ameliorate, and/or reduce the severity of cervical cancer or one or more symptoms thereof in the cervical cancer subject: (i) via a route selected from the group consisting of buccally, inhalation, intraarterially, intraarticularly, intracardially, intracerebroventricularly, intradermally, intramedullary, intramuscularly, intraperitoneally, intrathecally, intravenously, intraventricularly, nasally, ophthalmically, orally, rectally, sublabially, sublingually, subcutaneously, topically, and transdermally; and/or (ii) with a frequency selected from the group consisting of four times daily, three times daily, twice daily, once daily, once every two days, once every three days, once every four days, once every five days, once every six days, once a week, once every eight days, once every nine days, once every ten days, once every two weeks, once every three weeks, and once a month; and/or (iii) at a dosage (based on subject weight) selected from the group consisting of 0.1 μg/kg, 0.5 μg/kg, 1 μg/kg, 1.5 μg/kg, 2.0 μg/kg, 2.5 μg/kg, 3.0 μg/kg, 3.5 μg/kg, 4.0 μg/kg, 4.5 μg/kg, 5.0 μg/kg, 10 μg/kg, 15 μg/kg, 20 μg/kg, 25 μg/kg, 30 μg/kg, 35 μg/kg, 40 μg/kg, 45 μg/kg, 50 μg/kg, 55 μg/kg, 60 μg/kg, 65 μg/kg, 70 μg/kg, 75 μg/kg, 80 μg/kg, 85 μg/kg, 90 μg/kg, 95 μg/kg, 100 μg/kg, 150 μg/kg, 200 μg/kg, 250 μg/kg, 300 μg/kg, 350 μg/kg, 400 μg/kg, 450 μg/kg, 500 μg/kg, 550 μg/kg, 600 μg/kg, 650 μg/kg, 700 μg/kg, 750 μg/kg, 800 μg/kg, 850 μg/kg, 900 μg/kg, 1 mg/kg, 1.5 mg/kg, 2.0 mg/kg, 2.5 mg/kg, 3 mg/kg, 3.5 mg/kg, 4.0 mg/kg, 4.5 mg/kg, 5 mg/kg, 5.5 mg/kg, 6 mg/kg, 6.5 mg/kg, 7 mg/kg, 7.5 mg/kg, 8 mg/kg, 8.5 mg/kg, 9 mg/kg, 9.5 mg/kg, 10 mg/kg 10.5 mg/kg, 11 mg/kg, 11.5 mg/kg, 12 mg/kg, 12.5 mg/kg, 13 mg/kg, 13.5 mg/kg, 14 mg/kg, 14.5 mg/kg, 15 mg/kg, 16 mg/kg, 17 mg/kg, 18 mg/kg, 19 mg/kg, 20 mg/kg, 21 mg/kg, 22 mg/kg, 23 mg/kg, 24 mg/kg, 25 mg/kg, 30 mg/kg, 35 mg/kg, 40 mg/kg, 45 mg/kg, 50 mg/kg, 55 mg/kg, 60 mg/kg, 65 mg/kg, 70 mg/kg, 75 mg/kg, 80 mg/kg, 85 mg/kg, 90 mg/kg, 95 mg/kg, 100 mg/kg, 105 mg/kg, 110 mg/kg, 115 mg/kg, 120 mg/kg, 125 mg/kg, 130 mg/kg, 135 mg/kg, 140 mg/kg, 145 mg/kg, 150 mg/kg, 155 mg/kg, 160 mg/kg, 165 mg/kg, 170 mg/kg, 175 mg/kg, 180 mg/kg, 185 mg/kg, 190 mg/kg, 195 mg/kg, 200 mg/kg, and any fraction or integer in between any two of the preceding amounts.

In some embodiments, an nNOS inhibitor composition comprising one or more CRISPR/Cas systems, may be administered as a suitable dosage form, as described herein, to treat, ameliorate, and/or reduce the severity of cervical cancer or one or more symptoms thereof in a cervical cancer subject. In certain embodiments, an nNOS inhibitor composition comprising one or more CRISPR/Cas systems comprising an endonuclease selected from the group consisting of Cas1, Cas2, Cas3, Cas4, Cas5, Cas6, Cas7, Cas8a, Cas8b, Cas8c, Cas9, Cas10, Cas10d, Cas12, Cas12a, Cas12b, Cas12c, Cas12d, Cas12e, Cas12f, Cas12g, Cas12h, Cas12i, Cas12k, C2c4, C2c8, C2c9, Cas13, Cas13a, Cas13b, Cas13c, Cas13d, Cas13x.1, Cse1, Cse2, Csy1, Csy2, Csy3, GSU0054, Csm2, Cmr5, Csx10, Csx11, Csf1, and Csn2, may be administered as a suitable dosage form, as described herein, to treat, ameliorate, and/or reduce the severity of cervical cancer or one or more symptoms thereof in a cervical cancer subject. In some embodiments, an nNOS inhibitor composition comprising one or more CRISPR/Cas systems comprising an endonuclease selected from the group consisting of Cas1, Cas2, Cas3, Cas4, Cas5, Cas6, Cas7, Cas8a, Cas8b, Cas8c, Cas9, Cas10, Cas10d, Cas12, Cas12a, Cas12b, Cas12c, Cas12d, Cas12e, Cas12f, Cas12g, Cas12h, Cas12i, Cas12k, C2c4, C2c8, C2c9, Cas13, Cas13a, Cas13b, Cas13c, Cas13d, Cas13x.1, Cse1, Cse2, Csy1, Csy2, Csy3, GSU0054, Csm2, Cmr5, Csx10, Csx11, Csf1, and Csn2, may be administered to a cervical cancer subject to treat, ameliorate, and/or reduce the severity of cervical cancer or one or more symptoms thereof in the cervical cancer subject: (i) via a route selected from the group consisting of buccally, inhalation, intraarterially, intraarticularly, intracardially, intracerebroventricularly, intradermally, intramedullary, intramuscularly, intraperitoneally, intrathecally, intravenously, intraventricularly, nasally, ophthalmically, orally, rectally, sublabially, sublingually, subcutaneously, topically, and transdermally; and/or (ii) with a frequency selected from the group consisting of four times daily, three times daily, twice daily, once daily, once every two days, once every three days, once every four days, once every five days, once every six days, once a week, once every eight days, once every nine days, once every ten days, once every two weeks, once every three weeks, and once a month; and/or (iii) at a dosage (based on subject weight) selected from the group consisting of 0.1 μg/kg, 0.5 μg/kg, 1 μg/kg, 1.5 μg/kg, 2.0 μg/kg, 2.5 μg/kg, 3.0 μg/kg, 3.5 μg/kg, 4.0 μg/kg, 4.5 μg/kg, 5.0 μg/kg, 10 μg/kg, 15 μg/kg, 20 μg/kg, 25 μg/kg, 30 μg/kg, 35 μg/kg, 40 μg/kg, 45 μg/kg, 50 μg/kg, 55 μg/kg, 60 μg/kg, 65 μg/kg, 70 μg/kg, 75 μg/kg, 80 μg/kg, 85 μg/kg, 90 μg/kg, 95 μg/kg, 100 μg/kg, 150 μg/kg, 200 μg/kg, 250 μg/kg, 300 μg/kg, 350 μg/kg, 400 μg/kg, 450 μg/kg, 500 μg/kg, 550 μg/kg, 600 μg/kg, 650 μg/kg, 700 μg/kg, 750 μg/kg, 800 μg/kg, 850 μg/kg, 900 μg/kg, 1 mg/kg, 1.5 mg/kg, 2.0 mg/kg, 2.5 mg/kg, 3 mg/kg, 3.5 mg/kg, 4.0 mg/kg, 4.5 mg/kg, 5 mg/kg, 5.5 mg/kg, 6 mg/kg, 6.5 mg/kg, 7 mg/kg, 7.5 mg/kg, 8 mg/kg, 8.5 mg/kg, 9 mg/kg, 9.5 mg/kg, 10 mg/kg 10.5 mg/kg, 11 mg/kg, 11.5 mg/kg, 12 mg/kg, 12.5 mg/kg, 13 mg/kg, 13.5 mg/kg, 14 mg/kg, 14.5 mg/kg, 15 mg/kg, 16 mg/kg, 17 mg/kg, 18 mg/kg, 19 mg/kg, 20 mg/kg, 21 mg/kg, 22 mg/kg, 23 mg/kg, 24 mg/kg, 25 mg/kg, 30 mg/kg, 35 mg/kg, 40 mg/kg, 45 mg/kg, 50 mg/kg, 55 mg/kg, 60 mg/kg, 65 mg/kg, 70 mg/kg, 75 mg/kg, 80 mg/kg, 85 mg/kg, 90 mg/kg, 95 mg/kg, 100 mg/kg, 105 mg/kg, 110 mg/kg, 115 mg/kg, 120 mg/kg, 125 mg/kg, 130 mg/kg, 135 mg/kg, 140 mg/kg, 145 mg/kg, 150 mg/kg, 155 mg/kg, 160 mg/kg, 165 mg/kg, 170 mg/kg, 175 mg/kg, 180 mg/kg, 185 mg/kg, 190 mg/kg, 195 mg/kg, 200 mg/kg, and any fraction or integer in between any two of the preceding amounts.

In some embodiments, an nNOS inhibitor composition comprising one or more antibodies, may be administered as a suitable dosage form, as described herein, to treat, ameliorate, and/or reduce the severity of cervical cancer or one or more symptoms thereof in a cervical cancer subject. In certain embodiments, an nNOS inhibitor composition comprising one or more antibodies selected from the group consisting of 37-2800 (from ThermoFisher), EP1855Y (from abeam), EPR 24351-6 (from abeam), and any combination thereof, may be administered as a suitable dosage form, as described herein, to treat, ameliorate, and/or reduce the severity of cervical cancer or one or more symptoms thereof in a cervical cancer subject. In some embodiments, an nNOS inhibitor composition comprising one or more antibodies selected from the group consisting of 37-2800 (from ThermoFisher), EP1855Y (from abeam), EPR 24351-6 (from abeam), and any combination thereof, may be administered to a cervical cancer subject to treat, ameliorate, and/or reduce the severity of cervical cancer or one or more symptoms thereof in the cervical cancer subject: (i) via a route selected from the group consisting of buccally, inhalation, intraarterially, intraarticularly, intracardially, intracerebroventricularly, intradermally, intramedullary, intramuscularly, intraperitoneally, intrathecally, intravenously, intraventricularly, nasally, ophthalmically, orally, rectally, sublabially, sublingually, subcutaneously, topically, and transdermally; and/or (ii) with a frequency selected from the group consisting of four times daily, three times daily, twice daily, once daily, once every two days, once every three days, once every four days, once every five days, once every six days, once a week, once every eight days, once every nine days, once every ten days, once every two weeks, once every three weeks, and once a month; and/or (iii) at a dosage (based on subject weight) selected from the group consisting of 0.1 μg/kg, 0.5 μg/kg, 1 μg/kg, 1.5 μg/kg, 2.0 μg/kg, 2.5 μg/kg, 3.0 μg/kg, 3.5 μg/kg, 4.0 μg/kg, 4.5 μg/kg, 5.0 μg/kg, 10 μg/kg, 15 μg/kg, 20 μg/kg, 25 μg/kg, 30 μg/kg, 35 μg/kg, 40 μg/kg, 45 μg/kg, 50 μg/kg, 55 μg/kg, 60 μg/kg, 65 μg/kg, 70 μg/kg, 75 μg/kg, 80 μg/kg, 85 μg/kg, 90 μg/kg, 95 μg/kg, 100 μg/kg, 150 μg/kg, 200 μg/kg, 250 μg/kg, 300 μg/kg, 350 μg/kg, 400 μg/kg, 450 μg/kg, 500 μg/kg, 550 μg/kg, 600 μg/kg, 650 μg/kg, 700 μg/kg, 750 μg/kg, 800 μg/kg, 850 μg/kg, 900 μg/kg, 1 mg/kg, 1.5 mg/kg, 2.0 mg/kg, 2.5 mg/kg, 3 mg/kg, 3.5 mg/kg, 4.0 mg/kg, 4.5 mg/kg, 5 mg/kg, 5.5 mg/kg, 6 mg/kg, 6.5 mg/kg, 7 mg/kg, 7.5 mg/kg, 8 mg/kg, 8.5 mg/kg, 9 mg/kg, 9.5 mg/kg, 10 mg/kg 10.5 mg/kg, 11 mg/kg, 11.5 mg/kg, 12 mg/kg, 12.5 mg/kg, 13 mg/kg, 13.5 mg/kg, 14 mg/kg, 14.5 mg/kg, 15 mg/kg, 16 mg/kg, 17 mg/kg, 18 mg/kg, 19 mg/kg, 20 mg/kg, 21 mg/kg, 22 mg/kg, 23 mg/kg, 24 mg/kg, 25 mg/kg, 30 mg/kg, 35 mg/kg, 40 mg/kg, 45 mg/kg, 50 mg/kg, 55 mg/kg, 60 mg/kg, 65 mg/kg, 70 mg/kg, 75 mg/kg, 80 mg/kg, 85 mg/kg, 90 mg/kg, 95 mg/kg, 100 mg/kg, 105 mg/kg, 110 mg/kg, 115 mg/kg, 120 mg/kg, 125 mg/kg, 130 mg/kg, 135 mg/kg, 140 mg/kg, 145 mg/kg, 150 mg/kg, 155 mg/kg, 160 mg/kg, 165 mg/kg, 170 mg/kg, 175 mg/kg, 180 mg/kg, 185 mg/kg, 190 mg/kg, 195 mg/kg, 200 mg/kg, and any fraction or integer in between any two of the preceding amounts.

In some embodiments, an nNOS inhibitor composition comprising one or more amino acid-based compounds, may be administered as a suitable dosage form, as described herein, to treat, ameliorate, and/or reduce the severity of cervical cancer or one or more symptoms thereof in a cervical cancer subject. In certain embodiments, an nNOS inhibitor composition comprising one or more amino acid-compounds that bind an active site of nNOS selected from the group consisting of Phenylalanine 589, Tryptophan 592, Tyrosine 593, Glutamate 597, Aspartate 602, Arginine 601, Methionine 341, Histidine 342, and Tyrosine711, may be administered as a suitable dosage form, as described herein, to treat, ameliorate, and/or reduce the severity of cervical cancer or one or more symptoms thereof in a cervical cancer subject. In some embodiments, an nNOS inhibitor composition comprising one or more amino acid-compounds that bind an active site of nNOS selected from the group consisting of Phenylalanine 589, Tryptophan 592, Tyrosine 593, Glutamate 597, Aspartate 602, Arginine 601, Methionine 341, Histidine 342, and Tyrosine711, may be administered to a cervical cancer subject to treat, ameliorate, and/or reduce the severity of cervical cancer or one or more symptoms thereof in the cervical cancer subject: (i) via a route selected from the group consisting of buccally, inhalation, intraarterially, intraarticularly, intracardially, intracerebroventricularly, intradermally, intramedullary, intramuscularly, intraperitoneally, intrathecally, intravenously, intraventricularly, nasally, ophthalmically, orally, rectally, sublabially, sublingually, subcutaneously, topically, and transdermally; and/or (ii) with a frequency selected from the group consisting of four times daily, three times daily, twice daily, once daily, once every two days, once every three days, once every four days, once every five days, once every six days, once a week, once every eight days, once every nine days, once every ten days, once every two weeks, once every three weeks, and once a month; and/or (iii) at a dosage (based on subject weight) selected from the group consisting of 0.1 μg/kg, 0.5 μg/kg, 1 μg/kg, 1.5 μg/kg, 2.0 μg/kg, 2.5 μg/kg, 3.0 μg/kg, 3.5 μg/kg, 4.0 μg/kg, 4.5 μg/kg, 5.0 μg/kg, 10 μg/kg, 15 μg/kg, 20 μg/kg, 25 μg/kg, 30 μg/kg, 35 μg/kg, 40 μg/kg, 45 μg/kg, 50 μg/kg, 55 μg/kg, 60 μg/kg, 65 μg/kg, 70 μg/kg, 75 μg/kg, 80 μg/kg, 85 μg/kg, 90 μg/kg, 95 μg/kg, 100 μg/kg, 150 μg/kg, 200 μg/kg, 250 μg/kg, 300 μg/kg, 350 μg/kg, 400 μg/kg, 450 μg/kg, 500 μg/kg, 550 μg/kg, 600 μg/kg, 650 μg/kg, 700 μg/kg, 750 μg/kg, 800 μg/kg, 850 μg/kg, 900 μg/kg, 1 mg/kg, 1.5 mg/kg, 2.0 mg/kg, 2.5 mg/kg, 3 mg/kg, 3.5 mg/kg, 4.0 mg/kg, 4.5 mg/kg, 5 mg/kg, 5.5 mg/kg, 6 mg/kg, 6.5 mg/kg, 7 mg/kg, 7.5 mg/kg, 8 mg/kg, 8.5 mg/kg, 9 mg/kg, 9.5 mg/kg, 10 mg/kg 10.5 mg/kg, 11 mg/kg, 11.5 mg/kg, 12 mg/kg, 12.5 mg/kg, 13 mg/kg, 13.5 mg/kg, 14 mg/kg, 14.5 mg/kg, 15 mg/kg, 16 mg/kg, 17 mg/kg, 18 mg/kg, 19 mg/kg, 20 mg/kg, 21 mg/kg, 22 mg/kg, 23 mg/kg, 24 mg/kg, 25 mg/kg, 30 mg/kg, 35 mg/kg, 40 mg/kg, 45 mg/kg, 50 mg/kg, 55 mg/kg, 60 mg/kg, 65 mg/kg, 70 mg/kg, 75 mg/kg, 80 mg/kg, 85 mg/kg, 90 mg/kg, 95 mg/kg, 100 mg/kg, 105 mg/kg, 110 mg/kg, 115 mg/kg, 120 mg/kg, 125 mg/kg, 130 mg/kg, 135 mg/kg, 140 mg/kg, 145 mg/kg, 150 mg/kg, 155 mg/kg, 160 mg/kg, 165 mg/kg, 170 mg/kg, 175 mg/kg, 180 mg/kg, 185 mg/kg, 190 mg/kg, 195 mg/kg, 200 mg/kg, and any fraction or integer in between any two of the preceding amounts.

In certain embodiments, a cervical cancer subject, as described herein, may have a cervical cancer selected from the group consisting of an adenocarcinoma, an adenosquamous carcinoma, a carcinoma, a clear cell carcinoma, a glassy cell carcinoma, a lymphoma, a melanoma, a mixed carcinoma, a mucinous tumor, a neuroendocrine carcinoma, a sarcoma, a small cell carcinoma, or a squamous cell carcinoma.

In some embodiments, a cervical cancer subject, as described herein, may have an abnormal expression of one or more antigens compared to an equivalent subject that does not have cervical cancer. As used herein, the term “equivalent subject,” refers to a subject of the age range of the cervical cancer subject. For purposes of disclosure, the term “antigen,” when used with respect to abnormal expression, may define one or more CD antigens, such as, for example, CD15, CD44, CD70, CD133, as well as any CD antigen that has an abnormal expression in a cervical cancer disclosed herein that would be readily envisaged by one skilled in the art. In certain embodiments, abnormal expression of an antigen may define an abnormal expression of CD15, an abnormal expression of CD44, an abnormal expression of CD70, an abnormal expression of CD133, an abnormal expression of a CD antigen that has an abnormal expression in a cervical cancer disclosed herein that would be readily envisaged by one skilled in the art, and any combination thereof. In some embodiments, an “abnormal expression,” can refer to a reduced expression of an antigen, as described herein. In certain embodiments, an “abnormal expression,” can refer to an overexpression of an antigen, as described herein. Whether “abnormal expression,” refers to a reduced expression of an antigen or an overexpression of an antigen would be immediately envisaged by the skilled artisan when viewing the term in the context of the particular antigen disclosed, as well as the cervical cancer to which the abnormal expression of antigen refers to. For example, in the context of CD70 and cervical carcinoma, one skilled in the art would immediately envisage that “abnormal expression” refers to an overexpression of CD70. Treatment, amelioration, and/or reduction in the severity of a cervical cancer or one or more symptoms thereof may be characterized by a restoration to a normal level of expression of one or more antigens from their abnormal state, as described herein. For example, in the context of CD70 and cervical carcinoma, treatment, amelioration, and/or reduction in the severity of a cervical cancer or one or more symptoms thereof may be characterized by a reduced expression of CD70.

In certain embodiments, a cervical cancer subject, as described herein, may have an abnormal expression of one or more proteins compared to an equivalent subject that does not have cervical cancer. For purposes of disclosure, the term “protein,” when used with respect to abnormal expression, may define one or more proteins and/or enzymes, such as, for example, ALDH1A3, mTOR, p-mTOR, TSC-2, AKT, pAKT, RPS6, p-RPS6, Syp, VEGF, PIGF, as well as any protein or enzyme that has an abnormal expression in a cervical cancer disclosed herein that would be readily envisaged by one skilled in the art. In certain embodiments, abnormal expression of a protein may define an abnormal expression of ALDH1A3, an abnormal expression of mTOR, an abnormal expression of p-mTOR, an abnormal expression of TSC-2, an abnormal expression of AKT, an abnormal expression of pAKT, an abnormal expression of RPS6, an abnormal expression of p-RPS6, an abnormal expression of Syp, an abnormal expression of VEGF, an abnormal expression of PIGF, an abnormal expression of a protein or enzyme that has an abnormal expression in a cervical cancer disclosed herein that would be readily envisaged by one skilled in the art, and any combination thereof. In some embodiments, an “abnormal expression,” can refer to a reduced expression of a protein, as described herein. In certain embodiments, an “abnormal expression,” can refer to an overexpression of a protein, as described herein. Whether “abnormal expression,” refers to a reduced expression of a protein or an overexpression of a protein would be immediately envisaged by the skilled artisan when viewing the term in the context of the particular protein disclosed, as well as the cervical cancer to which the abnormal expression of protein refers to. For example, in the context of squamous cell carcinoma and mTOR, one skilled in the art would immediately envisage that “abnormal expression” refers to an overexpression of mTOR. Treatment, amelioration, and/or reduction in the severity of a cervical cancer or one or more symptoms thereof may be characterized by a restoration to a normal level of expression of one or more proteins from their abnormal state, as described herein. For example, in the context of squamous cell carcinoma and mTOR, treatment, amelioration, and/or reduction in the severity of a cervical cancer or one or more symptoms thereof may be characterized by a reduced expression of mTOR.

In certain embodiments, a cervical cancer subject, as described herein, may have an increased expression of one or more NOS isoforms compared to an equivalent subject that does not have cervical cancer. For purposes of disclosure, the term “NOS isoform,” refers to nNOS, iNOS, and/or eNOS. In certain embodiments, a cervical cancer subject, as described herein, may have an increased expression of nNOS compared to an equivalent subject that does not have cervical cancer. In some embodiments, a cervical cancer subject, as described herein, may have an increased expression of iNOS compared to an equivalent subject that does not have cervical cancer. In certain embodiments, a cervical cancer subject, as described herein, may have an increased expression of eNOS compared to an equivalent subject that does not have cervical cancer. In some embodiments, a cervical cancer subject, as described herein, may have an increased expression of nNOS and iNOS compared to an equivalent subject that does not have cervical cancer. In certain embodiments, a cervical cancer subject, as described herein, may have an increased expression of nNOS and eNOS compared to an equivalent subject that does not have cervical cancer. In some embodiments, a cervical cancer subject, as described herein, may have an increased expression of iNOS and eNOS compared to an equivalent subject that does not have cervical cancer. In certain embodiments, a cervical cancer subject, as described herein, may have an increased expression of nNOS, iNOS, and eNOS compared to an equivalent subject that does not have cervical cancer. Whether a “NOS isoform,” has an increased expression would be immediately envisaged by the skilled artisan when viewing the term in the context of the particular NOS isoform disclosed, as well as the cervical cancer to which the increased expression of NOS isoform refers to. For example, in the context of carcinoma, one skilled in the art would immediately envisage that iNOS has an increased expression. Treatment, amelioration, and/or reduction in the severity of a cervical cancer or one or more symptoms thereof may be characterized by a reduced expression of one or more NOS isoforms from their increased state, as described herein. For example, in the context of carcinoma, treatment, amelioration, and/or reduction in the severity of a cervical cancer or one or more symptoms thereof may be characterized by a reduced expression of iNOS. For purposes of disclosure, reduction/inhibition of the expression of one or more NOS isoforms may be characterized by a reduction in expression of one or more NOS isoforms by about at least 10%, about at least 20%, about at least 30%, or about at least 40%. In some embodiments, reduction/inhibition of the expression of one or more NOS isoforms may further be characterized by a lack reduction of other, non-implicated NOS isoforms, such as, for example, no more than about 10%, no more than about 5%, no more than about 2%, or no more than about 1% reduction/inhibition of the expression of non-implicated NOS isoforms. As used herein, the term “non-implicated NOS isoforms,” refers to NOS isoforms that do not have an increased expression in a particular cervical cancer. By way of example, in the context of carcinoma, if iNOS has an increased expression, nNOS and/or eNOS would define a “non-implicated” NOS isoforms. Whether a NOS isoform is a “non-implicated NOS isoform,” would be immediately envisaged by the skilled artisan when viewing the term in the context of the particular NOS isoform disclosed, as well as the cervical cancer.

In some embodiments, a cervical cancer subject, as described herein, may have an elevated level of one or more NO-indicators compared to an equivalent subject that does not have cervical cancer. As used herein, the term “NO-indicator,” can refer to 3-nitrotyrosine (3-Ntyr), NADPH-diaphorase, total nitrite concentration, total nitrate concentration, total nitrite and nitrate concentration, as well as any NO-indicator that has an elevated level in a cervical cancer disclosed herein that would be readily envisaged by one skilled in the art. For example, in the context of carcinoma, one skilled in the art would immediately envisage that 3-Ntyr can be present at an elevated level. Treatment, amelioration, and/or reduction in the severity of a cervical cancer or one or more symptoms thereof may be characterized by a reduction in the level of one or more NO-indicators. For example, in the context of carcinoma, treatment, amelioration, and/or reduction in the severity of a cervical cancer or one or more symptoms thereof may be characterized by a reduction in the level of 3-Ntyr.

In certain embodiments, a NOS inhibitor composition, as described herein, may be administered as a suitable dosage form, as described herein, to treat, ameliorate, and/or reduce the severity of a cervical cancer or one or more symptoms thereof, as an alternative to a conventional therapy for treating, ameliorating, and/or reducing the severity of a cervical cancer or one or more symptoms thereof. For purposes of disclosure, a “conventional therapy,” as described herein, may refer to a treatment that would be recommended by one skilled in the art to treat a cervical cancer, and may define one or more of radiation, chemotherapy, immunotherapy, cell-based therapy, gene therapy, and the like—one skilled in the art would immediately envisage a “conventional therapy” based on the particular cervical cancer disclosed, as well as characteristics of a cervical cancer subject, such as, for example, the subject's age, stage of the cervical cancer, genotype of the cervical cancer, and the like. Treatment, amelioration, and/or reduction in the severity of a cervical cancer or one or more symptoms thereof may be characterized by an improved outcome compared to a conventional therapy, as described herein. For purposes of the disclosure, an “improved outcome” may define any one of the following: (i) a larger reduction in tumor volume and/or mass compared to a subject administered an equivalent dose of a conventional therapy; (ii) an improvement in the expected rate of survival of a subject, at, for example, 1 month, 3 months, 6 months, 9 months, 1 year, 2 years, 3 years, 5 years, 10 years, or more, compared to the expected rate of survival of a subject administered an equivalent dose of a conventional therapy; (iii) a reduction in the prevalence of side effects resulting from the NOS inhibitor composition compared to a subject administered an equivalent dose of a conventional therapy; (iv) a reduction in the severity of side effects resulting from the NOS inhibitor composition compared to a subject administered an equivalent dose of a conventional therapy; (v) a reduction in the dosage required to achieve an equivalent reduction in tumor volume compared to a subject administered a conventional therapy; (vi) a reduction in the dosage required to achieve an equivalent expected rate of survival of a subject compared to a subject administered a conventional therapy; (vii) a reduction in the likelihood of a recurrence of the cervical cancer occurring in a subject compared to a subject administered an equivalent dose of a conventional therapy; (viii) a reduction in the likelihood of a metastasis occurring in a subject compared to a subject administered an equivalent dose of a conventional therapy; (ix) a reduction in the time required to be at a medical facility for treatment compared to a subject administered an equivalent dose of a conventional therapy; (x) a reduction in the cost associated with treatment compared to a subject administered an equivalent dose of a conventional therapy; as well as any other improved outcome not expressly recited that would be readily envisaged by one skilled in the art.

In some embodiments, a NOS inhibitor composition, as described herein, may be administered as a suitable dosage form, as described herein, to treat, ameliorate, and/or reduce the severity of a cervical cancer or one or more symptoms thereof, wherein the NOS inhibitor composition comprises a first chemical NOS inhibitor and a second chemical NOS inhibitor. In certain embodiments, the first chemical NOS inhibitor may be administered concurrently with the second chemical NOS inhibitor. In some embodiments, the first chemical NOS inhibitor may be administered prior to the second chemical NOS inhibitor. In certain embodiments, the first chemical NOS inhibitor may be administered subsequently to the second chemical NOS inhibitor. Treatment, amelioration, and/or reduction in the severity of a cervical cancer or one or more symptoms thereof may be characterized by an improved outcome compared an alternative treatment. As used herein, the term “alternative treatment,” may define one or more of a conventional therapy, as described herein, a NOS inhibitor composition comprising a single chemical NOS inhibitor, as described herein, and a NOS inhibitor composition comprising a single biological NOS inhibitor, as described herein. For purposes of the disclosure, an “improved outcome” may define any one of the following: (i) a larger reduction in tumor volume and/or mass compared an alternative treatment; (ii) an improvement in the expected rate of survival of a subject, at, for example, 1 month, 3 months, 6 months, 9 months, 1 year, 2 years, 3 years, 5 years, 10 years, or more, compared to the expected rate of survival of a subject administered an alternative treatment; (iii) a reduction in the prevalence of side effects resulting from the NOS inhibitor composition comprising at least two chemical NOS inhibitors compared to a subject administered an alternative treatment; (iv) a reduction in the severity of side effects resulting from the NOS inhibitor composition comprising at least two chemical NOS inhibitors compared to a subject administered an alternative treatment; (v) a reduction in the dosage required to achieve an equivalent reduction in tumor volume compared to a subject administered an alternative treatment; (vi) a reduction in the dosage required to achieve an equivalent expected rate of survival of a subject compared to a subject administered an alternative treatment; (vii) a reduction in the likelihood of a recurrence of the cervical cancer in a subject compared to a subject administered an alternative treatment; (viii) a reduction in the likelihood of a metastasis occurring in a subject compared to a subject administered an alternative treatment; (ix) a reduction in the time required to be at a medical facility for treatment compared to a subject administered an alternative treatment; (x) a reduction in the cost associated with treatment compared to a subject administered an alternative treatment; as well as any other improved outcome not expressly recited that would be readily envisaged by one skilled in the art.

In certain embodiments, a NOS inhibitor composition, as described herein, may be used to treat, ameliorate, and/or reduce the severity of a cervical cancer or one or more symptoms thereof, wherein the NOS inhibitor composition comprises at least one chemical NOS inhibitor and at least one biological NOS inhibitor. In some embodiments, the at least one chemical NOS inhibitor may be administered concurrently with the at least one biological NOS inhibitor. In certain embodiments, the at least one chemical NOS inhibitor may be administered prior to the at least one biological NOS inhibitor. In some embodiments, the at least one chemical NOS inhibitor may be administered subsequently to the at least one biological NOS inhibitor. Treatment, amelioration, and/or reduction in the severity of a cervical cancer or one or more symptoms thereof may be characterized by an improved outcome compared an alternative treatment. As used herein, the term “alternative treatment,” may define one or more of a conventional therapy, as described herein, a NOS inhibitor composition comprising a single chemical NOS inhibitor, as described herein, and a NOS inhibitor composition comprising a single biological NOS inhibitor, as described herein. For purposes of the disclosure, an “improved outcome” may define any one of the following: (i) a larger reduction in tumor volume and/or mass compared an alternative treatment; (ii) an improvement in the expected rate of survival of a subject, at, for example, 1 month, 3 months, 6 months, 9 months, 1 year, 2 years, 3 years, 5 years, 10 years, or more, compared to the expected rate of survival of a subject administered an alternative treatment; (iii) a reduction in the prevalence of side effects resulting from the NOS inhibitor composition comprising at least one chemical NOS inhibitor and at least one biological NOS inhibitor compared to a subject administered an alternative treatment; (iv) a reduction in the severity of side effects resulting from the NOS inhibitor composition comprising at least one chemical NOS inhibitor and at least one biological NOS inhibitor compared to a subject administered an alternative treatment; (v) a reduction in the dosage required to achieve an equivalent reduction in tumor volume compared to a subject administered an alternative treatment; (vi) a reduction in the dosage required to achieve an equivalent expected rate of survival of a subject compared to a subject administered an alternative treatment; (vii) a reduction in the likelihood of a recurrence of the cervical cancer in a subject compared to a subject administered an alternative treatment; (viii) a reduction in the likelihood of a metastasis occurring in a subject compared to a subject administered an alternative treatment; (ix) a reduction in the time required to be at a medical facility for treatment compared to a subject administered an alternative treatment; (x) a reduction in the cost associated with treatment compared to a subject administered an alternative treatment; as well as any other improved outcome not expressly recited that would be readily envisaged by one skilled in the art.

In some embodiments, a NOS inhibitor composition, as described herein, may be combined with a conventional therapy, as described herein, to treat, ameliorate, and/or reduce the severity of a cervical cancer or one or more symptoms thereof. In certain embodiments, the NOS inhibitor composition may be administered concurrently with the conventional therapy. In some embodiments, the NOS inhibitor composition may be administered prior to the conventional therapy. In certain embodiments, the NOS inhibitor composition may be administered subsequently to the conventional therapy. Treatment, amelioration, and/or reduction in the severity of a cervical cancer or one or more symptoms thereof may be characterized by an improved outcome compared an alternative treatment. As used herein, the term “alternative treatment,” may define one or more of a conventional therapy, as described herein, a NOS inhibitor composition comprising a single chemical NOS inhibitor, as described herein, and a NOS inhibitor composition comprising a single biological NOS inhibitor, as described herein. For purposes of the disclosure, an “improved outcome” may define any one of the following: (i) a larger reduction in tumor volume and/or mass compared an alternative treatment; (ii) an improvement in the expected rate of survival of a subject, at, for example, 1 month, 3 months, 6 months, 9 months, 1 year, 2 years, 3 years, 5 years, 10 years, or more, compared to the expected rate of survival of a subject administered an alternative treatment; (iii) a reduction in the prevalence of side effects resulting from the NOS inhibitor composition and conventional therapy compared to a subject administered an alternative treatment; (iv) a reduction in the severity of side effects resulting from the NOS inhibitor composition and conventional therapy compared to a subject administered an alternative treatment; (v) a reduction in the dosage required to achieve an equivalent reduction in tumor volume compared to a subject administered an alternative treatment; (vi) a reduction in the dosage required to achieve an equivalent expected rate of survival of a subject compared to a subject administered an alternative treatment; (vii) a reduction in the likelihood of a recurrence of the cervical cancer in a subject compared to a subject administered an alternative treatment; (viii) a reduction in the likelihood of a metastasis occurring in a subject compared to a subject administered an alternative treatment; (ix) a reduction in the time required to be at a medical facility for treatment compared to a subject administered an alternative treatment; (x) a reduction in the cost associated with treatment compared to a subject administered an alternative treatment; as well as any other improved outcome not expressly recited that would be readily envisaged by one skilled in the art.

In some embodiments, a “conventional therapy,” as that term is used with respect to combined administration with a NOS inhibitor composition, may refer to a conventional chemotherapy. For purposes of the disclosure, a “conventional chemotherapy,” as described herein may be a compound selected from the group consisting of abemaciclib, abiraterone acetate, acalabrutinib, adagrasib, afatinib dimaleate, albumin-bound paclitaxel, albumin-bound rapamycin (Sirolimus Protein-Bound Particles), alectinib, alpelisib, amifostine, aminolevulinic acid hydrochloride, anastrozole, apalutamide, aprepitant, arsenic trioxide, asciminib hydrochloride, avapritinib, axitinib, azacytidine, belinostat, belzutifan, bendamustine hydrochloride, bexarotene, bicalutamide, binimetinib, bleomycin, bleomycin sulfate, bortezomib, bosutinib, brigatinib, busulfan, cabazitaxel, cabozantinib-S-malate, capecitabine, capmatinib hydrochloride, carboplatin, carfilzomib, carmustine, cedazuridine, ceritinib, chlorambucil, cisplatin, cladribine, clofarabine, cobimetinib fumarate, copanlisib hydrochloride, crizotinib, cyclophosphamide, cytarabine, dabrafenib mesylate, dacarbazine, dacomitinib, dactinomycin, darolutamide, dasatinib, daunorubicin hydrochloride, decitabine, defibrotide sodium, degarelix, dexamethasone, dexrazoxane hydrochloride, docetaxel, doxorubicin hydrochloride, duvelisib, elacestrant dihydrochloride, eltrombopag olamine, enasidenib mesylate, encorafenib, entrectinib, enzalutamide, epirubicin hydrochloride, erdafitinib, eribulin mesylate, erlotinib hydrochloride, etoposide, etoposide phosphate, everolimus, exemestane, fedratinib hydrochloride, fludarabine phosphate, fluorouracil, flutamide, folinic acid, fostamatinib disodium, fulvestrant, futibatinib, gefitinib, gemcitabine hydrochloride, gilteritinib fumarate, glasdegib maleate, goserelin acetate, granisetron, granisetron hydrochloride, hydroxyurea, ibrutinib, idarubicin hydrochloride, idelalisib, ifosfamide, imatinib mesylate, imiquimod, infigratinib phosphate, iobenguane I-131, irinotecan hydrochloride, ivosidenib, ixabepilone, ixazomib citrate, lanreotide acetate, lapatinib ditosylate, larotrectinib sulfate, lenalidomide, lenvatinib mesylate, letrozole, leucovorin calcium, leuprolide acetate, liposomal cytarabine, liposomal doxorubicin hydrochloride, liposomal irinotecan hydrochloride, lomustine, lorlatinib, lurbinectedin, Lu-177 dotatate, Lu-177 vipivotide tetraxetan, mechlorethamine hydrochloride, megestrol acetate, melphalan, melphalan hydrochloride, mercaptopurine, mesna, methotrexate, methotrexate sodium, methylnaltrexone bromide, midostaurin, mitomycin, mitoxantrone hydrochloride, mobocertinib succinate, nelarabine, neratinib maleate, netupitant hydrochloride, nilutamide, nilotinib, niraparib tosylate monohydrate, olaparib, olutasidenib, omacetaxine mepesuccinate, ondansetron hydrochloride, osimertinib mesylate, oxaliplatin, paclitaxel, pacritinib citrate, palbociclib, palonosetron hydrochloride, pamidronate disodium, pazopanib hydrochloride, pemetrexed disodium, pemigatinib, pexidartinib hydrochloride, pirtobrutinib, plerixafor, pomalidomide, ponatinib hydrochloride, pralatrexate, pralsetinib, prednisone, procarbazine hydrochloride, propranolol hydrochloride, Ra-223 dichloride, raloxifene hydrochloride, regorafenib, relugolix, ribociclib, ripretinib, rolapitant hydrochloride, romidepsin, rucaparib camsylate, ruxolitinib phosphate, selinexor, selpercatinib, selumetinib sulfate, sonidegib, sorafenib tosylate, sotorasib, sunitinib malate, talazoparib tosylate, talc, tamoxifen citrate, tazemetostat hydrobromide, temozolomide, temsirolimus, tepotinib hydrochloride, thalidomide, thioguanine, thiotepa, tipiracil hydrochloride, tivozanib hydrochloride, topotecan hydrochloride, toremifene, trabectedin, trametinib dimethyl sulfoxide, tretinoin, trifluridine hydrochloride, tucatinib, uridine triacetate, valrubicin, vandetanib, vemurafenib, venetoclax, vinblastine sulfate, vincristine sulfate, vinorelbine tartrate, vismodegib, vorinostat, zanubrutinib, zoledronic acid, and any combination thereof. One skilled in the art would immediately envisage an appropriate “conventional chemotherapy” based on the particular cervical cancer disclosed, as well as characteristics of a cervical cancer subject, such as, for example, the subject's age, stage of the cervical cancer, genotype of the cervical cancer, and the like.

In certain embodiments, a “conventional therapy,” as that term is used with respect to combined administration with a NOS inhibitor composition, may refer to a conventional immunotherapy. For purposes of the disclosure, a “conventional immunotherapy,” as described herein may be an immunotherapy, cell-based therapy, and/or gene-based therapy selected from the group consisting of ado-trastuzumab emtansine, aldesleukin, alemtuzumab, amivantamab-vmjw, asparaginase Erwinia chrysanthemi, asparaginase Erwinia chrysanthemi (recombinant)-rywn, atezolizumab, avelumab, axicabtagene ciloleucel, bevacizumab, blinatumomab, brentuximab vedotin, brexucabtagene autoleucel, calaspargase pegol-mknl, camrelizumab, caplacizumab-yhdp, cemiplimab-rwlc, cetuximab, ciltacabtagene autoleucel, daratumumab, daratumumab and hyaluronidase-fihj, darbepoetin alfa, denileukin diftitox, denosumab, dinutuximab, dostarlimab-gxly, durvalumab, elotuzumab, emapalumab-lzsg, enfortumab vedotin-ejfv, epoetin alfa, fam-trastuzumab deruxtecan-nxki, filgrastim, gemtuzumab ozogamicin, glucarpidase, ibritumomab tiuxetan, idecabtagene vicleucel, inotuzumab ozogamicin, ipilimumab, isatuximab-irfc, lisocabtagene maraleucel, loncastuximab tesirine-lpyl, luspatercept-aamt, margetuximab-cmkb, mirvetuximab soravtansine-gynx, mogamulizumab-kpkc, mosunetuzumab-axgb, moxetumomab pasudotox-tdfk, nadofaragene firadenovec-vncg, naxitamab-gqgk, necitumumab, nivolumab, nivolumab and relatlimab-rmbw, obinutuzumab, ofatumumab, palifermin, panitumumab, pegaspargase, pegfilgrastim, pembrolizumab, pertuzumab, pertuzumab, trastuzumab, and hyaluronidase-zzxf, polatuzumab vedotin-piiq, ramucirumab, rasburicase, ravulizumab-cwvz, recombinant HPV bivalent vaccine, recombinant HPV nonavalent vaccine, recombinant HPV quadrivalent vaccine, recombinant interferon alfa-2b, rituximab, rituximab and hyaluronidase human, romiplostim, ropeginterferon alfa-2b-njft, sacituzumab govitecan-hziy, siltuximab, sintilimab, sipuleucel-T, tafasitamab-cxix, tagraxofusp-erzs, talimogene laherparepvec, tebentafusp-tebn, teclistamab-cqyv, tisagenlecleucel, tislelizumab, tisotumab vedotin-tftv, tocilizumab, toripalimab, trastuzumab, trastuzumab and hyaluronidase-oysk, tremelimumab-actl, ziv-aflibercept, and any combination thereof. One skilled in the art would immediately envisage an appropriate “conventional immunotherapy” based on the particular cervical cancer disclosed, as well as characteristics of a cervical cancer subject, such as, for example, the subject's age, stage of the cervical cancer, genotype of the cervical cancer, and the like.

In certain embodiments, a NOS inhibitor composition, as described herein, may be administered as a suitable dosage form, as described herein, prior to, or alternatively, following a surgery to remove or resect a cervical cancer.

In some embodiments, a NOS inhibitor composition, as described herein, may be administered as a suitable dosage form, as described herein, to restore and/or maintain healthy and/or optimal expression levels of one or more antigens, as described herein, such as, for example, CD15, CD44, CD70, CD133, as well as any CD antigen that has an abnormal expression in a cervical cancer disclosed herein that would be readily envisaged by one skilled in the art, in a subject. In certain embodiments, the subject may have a cervical cancer or one or more symptoms thereof. As used herein, the terms “healthy” and “optimal” refer to expression levels of one or more antigens that would be typical of an equivalent subject that does not have cervical cancer.

In certain embodiments, a NOS inhibitor composition, as described herein, may be administered as a suitable dosage form, as described herein, to restore and/or maintain healthy and/or optimal expression levels of one or more proteins, as described herein, such as, for example, ALDH1A3, mTOR, p-mTOR, TSC-2, AKT, pAKT, RPS6, p-RPS6, Syp, VEGF, PIGF, as well as any protein or enzyme that has an abnormal expression in a cervical cancer disclosed herein that would be readily envisaged by one skilled in the art, in a subject. In certain embodiments, the subject may have a cervical cancer or one or more symptoms thereof. As used herein, the terms “healthy” and “optimal” refer to expression levels of one or more proteins that would be typical of an equivalent subject that does not have cervical cancer.

In some embodiments, a NOS inhibitor composition, as described herein, may be administered as a suitable dosage form, as described herein, to restore and/or maintain healthy and/or optimal expression levels of one or more NOS isoforms, as described herein, such as, for example, nNOS, iNOS, and eNOS, in a subject. In certain embodiments, the subject may have a cervical cancer or one or more symptoms thereof. As used herein, the terms “healthy” and “optimal” refer to expression levels of one or more NOS isoforms that would be typical of an equivalent subject that does not have cervical cancer.

In certain embodiments, a NOS inhibitor composition, as described herein, may be administered as a suitable dosage form, as described herein, to restore and/or maintain healthy and/or optimal levels of one or more NO-indicators, as described herein, such as, for example, 3-Ntyr, NADPH-diaphorase, total nitrite concentration, total nitrate concentration, total nitrite and nitrate concentration, as well as any NO-indicator that has an elevated level in a cervical cancer disclosed herein that would be readily envisaged by one skilled in the art, in a subject. In certain embodiments, the subject may have a cervical cancer or one or more symptoms thereof. As used herein, the terms “healthy” and “optimal” refer to levels of one or more NO-indicators that would be typical of an equivalent subject that does not have cervical cancer.

In certain embodiments, a NOS inhibitor composition, as described herein, may be administered as a suitable dosage form, as described herein, to treat, ameliorate, and/or reduce the severity of a lung cancer associated with an upregulation of NO signaling (“NO-lung cancer”) or one or more symptoms thereof in a subject (“NO-lung cancer subject”). One skilled in the art would readily envisage the subtypes of lung cancer that are associated with an upregulation of NO signaling.

In some embodiments, a NOS inhibitor composition comprising 7-NI and/or a salt thereof may be administered as a suitable dosage form, as described herein, to treat, ameliorate, and/or reduce the severity of NO-lung cancer or one or more symptoms thereof in an NO-lung cancer subject. In certain embodiments, an nNOS inhibitor composition comprising 7-NI and/or a salt thereof may be administered to an NO-lung cancer subject to treat, ameliorate, and/or reduce the severity of NO-lung cancer or one or more symptoms thereof in the NO-lung cancer subject: (i) via a route selected from the group consisting of buccally, inhalation, intraarterially, intraarticularly, intracardially, intracerebroventricularly, intradermally, intramedullary, intramuscularly, intraperitoneally, intrathecally, intravenously, intraventricularly, nasally, ophthalmically, orally, rectally, sublabially, sublingually, subcutaneously, topically, and transdermally; and/or (ii) with a frequency selected from the group consisting of four times daily, three times daily, twice daily, once daily, once every two days, once every three days, once every four days, once every five days, once every six days, once a week, once every eight days, once every nine days, once every ten days, once every two weeks, once every three weeks, and once a month; and/or (iii) at a dosage (based on subject weight) selected from the group consisting of 0.1 μg/kg, 0.5 μg/kg, 1 μg/kg, 1.5 μg/kg, 2.0 μg/kg, 2.5 μg/kg, 3.0 μg/kg, 3.5 μg/kg, 4.0 μg/kg, 4.5 μg/kg, 5.0 μg/kg, 10 μg/kg, 15 μg/kg, 20 μg/kg, 25 μg/kg, 30 μg/kg, 35 μg/kg, 40 μg/kg, 45 μg/kg, 50 μg/kg, 55 μg/kg, 60 μg/kg, 65 μg/kg, 70 μg/kg, 75 μg/kg, 80 μg/kg, 85 μg/kg, 90 μg/kg, 95 μg/kg, 100 μg/kg, 150 μg/kg, 200 μg/kg, 250 μg/kg, 300 μg/kg, 350 μg/kg, 400 μg/kg, 450 μg/kg, 500 μg/kg, 550 μg/kg, 600 μg/kg, 650 μg/kg, 700 μg/kg, 750 μg/kg, 800 μg/kg, 850 μg/kg, 900 μg/kg, 1 mg/kg, 1.5 mg/kg, 2.0 mg/kg, 2.5 mg/kg, 3 mg/kg, 3.5 mg/kg, 4.0 mg/kg, 4.5 mg/kg, 5 mg/kg, 5.5 mg/kg, 6 mg/kg, 6.5 mg/kg, 7 mg/kg, 7.5 mg/kg, 8 mg/kg, 8.5 mg/kg, 9 mg/kg, 9.5 mg/kg, 10 mg/kg 10.5 mg/kg, 11 mg/kg, 11.5 mg/kg, 12 mg/kg, 12.5 mg/kg, 13 mg/kg, 13.5 mg/kg, 14 mg/kg, 14.5 mg/kg, 15 mg/kg, 16 mg/kg, 17 mg/kg, 18 mg/kg, 19 mg/kg, 20 mg/kg, 21 mg/kg, 22 mg/kg, 23 mg/kg, 24 mg/kg, 25 mg/kg, 30 mg/kg, 35 mg/kg, 40 mg/kg, 45 mg/kg, 50 mg/kg, 55 mg/kg, 60 mg/kg, 65 mg/kg, 70 mg/kg, 75 mg/kg, 80 mg/kg, 85 mg/kg, 90 mg/kg, 95 mg/kg, 100 mg/kg, 105 mg/kg, 110 mg/kg, 115 mg/kg, 120 mg/kg, 125 mg/kg, 130 mg/kg, 135 mg/kg, 140 mg/kg, 145 mg/kg, 150 mg/kg, 155 mg/kg, 160 mg/kg, 165 mg/kg, 170 mg/kg, 175 mg/kg, 180 mg/kg, 185 mg/kg, 190 mg/kg, 195 mg/kg, 200 mg/kg, and any fraction or integer in between any two of the preceding amounts.

In certain embodiments, a NOS inhibitor composition comprising 3-Br-7-NI and/or a salt thereof may be administered as a suitable dosage form, as described herein, to treat, ameliorate, and/or reduce the severity of NO-lung cancer or one or more symptoms thereof in an NO-lung cancer subject. In some embodiments, an nNOS inhibitor composition comprising 3-Br-7-NI and/or a salt thereof may be administered to an NO-lung cancer subject to treat, ameliorate, and/or reduce the severity of NO-lung cancer or one or more symptoms thereof in the NO-lung cancer subject: (i) via a route selected from the group consisting of buccally, inhalation, intraarterially, intraarticularly, intracardially, intracerebroventricularly, intradermally, intramedullary, intramuscularly, intraperitoneally, intrathecally, intravenously, intraventricularly, nasally, ophthalmically, orally, rectally, sublabially, sublingually, subcutaneously, topically, and transdermally; and/or (ii) with a frequency selected from the group consisting of four times daily, three times daily, twice daily, once daily, once every two days, once every three days, once every four days, once every five days, once every six days, once a week, once every eight days, once every nine days, once every ten days, once every two weeks, once every three weeks, and once a month; and/or (iii) at a dosage (based on subject weight) selected from the group consisting of 0.1 μg/kg, 0.5 μg/kg, 1 μg/kg, 1.5 μg/kg, 2.0 μg/kg, 2.5 μg/kg, 3.0 μg/kg, 3.5 μg/kg, 4.0 μg/kg, 4.5 μg/kg, 5.0 μg/kg, 10 μg/kg, 15 μg/kg, 20 μg/kg, 25 μg/kg, 30 μg/kg, 35 μg/kg, 40 μg/kg, 45 μg/kg, 50 μg/kg, 55 μg/kg, 60 μg/kg, 65 μg/kg, 70 μg/kg, 75 μg/kg, 80 μg/kg, 85 μg/kg, 90 μg/kg, 95 μg/kg, 100 μg/kg, 150 μg/kg, 200 μg/kg, 250 μg/kg, 300 μg/kg, 350 μg/kg, 400 μg/kg, 450 μg/kg, 500 μg/kg, 550 μg/kg, 600 μg/kg, 650 μg/kg, 700 μg/kg, 750 μg/kg, 800 μg/kg, 850 μg/kg, 900 μg/kg, 1 mg/kg, 1.5 mg/kg, 2.0 mg/kg, 2.5 mg/kg, 3 mg/kg, 3.5 mg/kg, 4.0 mg/kg, 4.5 mg/kg, 5 mg/kg, 5.5 mg/kg, 6 mg/kg, 6.5 mg/kg, 7 mg/kg, 7.5 mg/kg, 8 mg/kg, 8.5 mg/kg, 9 mg/kg, 9.5 mg/kg, 10 mg/kg 10.5 mg/kg, 11 mg/kg, 11.5 mg/kg, 12 mg/kg, 12.5 mg/kg, 13 mg/kg, 13.5 mg/kg, 14 mg/kg, 14.5 mg/kg, 15 mg/kg, 16 mg/kg, 17 mg/kg, 18 mg/kg, 19 mg/kg, 20 mg/kg, 21 mg/kg, 22 mg/kg, 23 mg/kg, 24 mg/kg, 25 mg/kg, 30 mg/kg, 35 mg/kg, 40 mg/kg, 45 mg/kg, 50 mg/kg, 55 mg/kg, 60 mg/kg, 65 mg/kg, 70 mg/kg, 75 mg/kg, 80 mg/kg, 85 mg/kg, 90 mg/kg, 95 mg/kg, 100 mg/kg, 105 mg/kg, 110 mg/kg, 115 mg/kg, 120 mg/kg, 125 mg/kg, 130 mg/kg, 135 mg/kg, 140 mg/kg, 145 mg/kg, 150 mg/kg, 155 mg/kg, 160 mg/kg, 165 mg/kg, 170 mg/kg, 175 mg/kg, 180 mg/kg, 185 mg/kg, 190 mg/kg, 195 mg/kg, 200 mg/kg, and any fraction or integer in between any two of the preceding amounts.

In some embodiments, a NOS inhibitor composition comprising L-NPA and/or a salt thereof, such as, for example, L-NPA hydrochloride, may be administered as a suitable dosage form, as described herein, to treat, ameliorate, and/or reduce the severity of NO-lung cancer or one or more symptoms thereof in an NO-lung cancer subject. In certain embodiments, an nNOS inhibitor composition comprising L-NPA and/or a salt thereof, such as, for example, L-NPA hydrochloride may be administered to an NO-lung cancer subject to treat, ameliorate, and/or reduce the severity of NO-lung cancer or one or more symptoms thereof in the NO-lung cancer subject: (i) via a route selected from the group consisting of buccally, inhalation, intraarterially, intraarticularly, intracardially, intracerebroventricularly, intradermally, intramedullary, intramuscularly, intraperitoneally, intrathecally, intravenously, intraventricularly, nasally, ophthalmically, orally, rectally, sublabially, sublingually, subcutaneously, topically, and transdermally; and/or (ii) with a frequency selected from the group consisting of four times daily, three times daily, twice daily, once daily, once every two days, once every three days, once every four days, once every five days, once every six days, once a week, once every eight days, once every nine days, once every ten days, once every two weeks, once every three weeks, and once a month; and/or (iii) at a dosage (based on subject weight) selected from the group consisting of 0.1 μg/kg, 0.5 μg/kg, 1 μg/kg, 1.5 μg/kg, 2.0 μg/kg, 2.5 μg/kg, 3.0 μg/kg, 3.5 μg/kg, 4.0 μg/kg, 4.5 μg/kg, 5.0 μg/kg, 10 μg/kg, 15 μg/kg, 20 μg/kg, 25 μg/kg, 30 μg/kg, 35 μg/kg, 40 μg/kg, 45 μg/kg, 50 μg/kg, 55 μg/kg, 60 μg/kg, 65 μg/kg, 70 μg/kg, 75 μg/kg, 80 μg/kg, 85 μg/kg, 90 μg/kg, 95 μg/kg, 100 μg/kg, 150 μg/kg, 200 μg/kg, 250 μg/kg, 300 μg/kg, 350 μg/kg, 400 μg/kg, 450 μg/kg, 500 μg/kg, 550 μg/kg, 600 μg/kg, 650 μg/kg, 700 μg/kg, 750 μg/kg, 800 μg/kg, 850 μg/kg, 900 μg/kg, 1 mg/kg, 1.5 mg/kg, 2.0 mg/kg, 2.5 mg/kg, 3 mg/kg, 3.5 mg/kg, 4.0 mg/kg, 4.5 mg/kg, 5 mg/kg, 5.5 mg/kg, 6 mg/kg, 6.5 mg/kg, 7 mg/kg, 7.5 mg/kg, 8 mg/kg, 8.5 mg/kg, 9 mg/kg, 9.5 mg/kg, 10 mg/kg 10.5 mg/kg, 11 mg/kg, 11.5 mg/kg, 12 mg/kg, 12.5 mg/kg, 13 mg/kg, 13.5 mg/kg, 14 mg/kg, 14.5 mg/kg, 15 mg/kg, 16 mg/kg, 17 mg/kg, 18 mg/kg, 19 mg/kg, 20 mg/kg, 21 mg/kg, 22 mg/kg, 23 mg/kg, 24 mg/kg, 25 mg/kg, 30 mg/kg, 35 mg/kg, 40 mg/kg, 45 mg/kg, 50 mg/kg, 55 mg/kg, 60 mg/kg, 65 mg/kg, 70 mg/kg, 75 mg/kg, 80 mg/kg, 85 mg/kg, 90 mg/kg, 95 mg/kg, 100 mg/kg, 105 mg/kg, 110 mg/kg, 115 mg/kg, 120 mg/kg, 125 mg/kg, 130 mg/kg, 135 mg/kg, 140 mg/kg, 145 mg/kg, 150 mg/kg, 155 mg/kg, 160 mg/kg, 165 mg/kg, 170 mg/kg, 175 mg/kg, 180 mg/kg, 185 mg/kg, 190 mg/kg, 195 mg/kg, 200 mg/kg, and any fraction or integer in between any two of the preceding amounts.

In certain embodiments, a NOS inhibitor composition comprising L-NIO and/or a salt thereof, such as, for example, L-NIO dihydrochloride, may be administered as a suitable dosage form, as described herein, to treat, ameliorate, and/or reduce the severity of NO-lung cancer or one or more symptoms thereof in an NO-lung cancer subject. In some embodiments, an nNOS inhibitor composition comprising L-NIO and/or a salt thereof, such as, for example, L-NIO dihydrochloride, may be administered to an NO-lung cancer subject to treat, ameliorate, and/or reduce the severity of NO-lung cancer or one or more symptoms thereof in the NO-lung cancer subject: (i) via a route selected from the group consisting of buccally, inhalation, intraarterially, intraarticularly, intracardially, intracerebroventricularly, intradermally, intramedullary, intramuscularly, intraperitoneally, intrathecally, intravenously, intraventricularly, nasally, ophthalmically, orally, rectally, sublabially, sublingually, subcutaneously, topically, and transdermally; and/or (ii) with a frequency selected from the group consisting of four times daily, three times daily, twice daily, once daily, once every two days, once every three days, once every four days, once every five days, once every six days, once a week, once every eight days, once every nine days, once every ten days, once every two weeks, once every three weeks, and once a month; and/or (iii) at a dosage (based on subject weight) selected from the group consisting of 0.1 μg/kg, 0.5 μg/kg, 1 μg/kg, 1.5 μg/kg, 2.0 μg/kg, 2.5 μg/kg, 3.0 μg/kg, 3.5 μg/kg, 4.0 μg/kg, 4.5 μg/kg, 5.0 μg/kg, 10 μg/kg, 15 μg/kg, 20 μg/kg, 25 μg/kg, 30 μg/kg, 35 μg/kg, 40 μg/kg, 45 μg/kg, 50 μg/kg, 55 μg/kg, 60 μg/kg, 65 μg/kg, 70 μg/kg, 75 μg/kg, 80 μg/kg, 85 μg/kg, 90 μg/kg, 95 μg/kg, 100 μg/kg, 150 μg/kg, 200 μg/kg, 250 μg/kg, 300 μg/kg, 350 μg/kg, 400 μg/kg, 450 μg/kg, 500 μg/kg, 550 μg/kg, 600 μg/kg, 650 μg/kg, 700 μg/kg, 750 μg/kg, 800 μg/kg, 850 μg/kg, 900 μg/kg, 1 mg/kg, 1.5 mg/kg, 2.0 mg/kg, 2.5 mg/kg, 3 mg/kg, 3.5 mg/kg, 4.0 mg/kg, 4.5 mg/kg, 5 mg/kg, 5.5 mg/kg, 6 mg/kg, 6.5 mg/kg, 7 mg/kg, 7.5 mg/kg, 8 mg/kg, 8.5 mg/kg, 9 mg/kg, 9.5 mg/kg, 10 mg/kg 10.5 mg/kg, 11 mg/kg, 11.5 mg/kg, 12 mg/kg, 12.5 mg/kg, 13 mg/kg, 13.5 mg/kg, 14 mg/kg, 14.5 mg/kg, 15 mg/kg, 16 mg/kg, 17 mg/kg, 18 mg/kg, 19 mg/kg, 20 mg/kg, 21 mg/kg, 22 mg/kg, 23 mg/kg, 24 mg/kg, 25 mg/kg, 30 mg/kg, 35 mg/kg, 40 mg/kg, 45 mg/kg, 50 mg/kg, 55 mg/kg, 60 mg/kg, 65 mg/kg, 70 mg/kg, 75 mg/kg, 80 mg/kg, 85 mg/kg, 90 mg/kg, 95 mg/kg, 100 mg/kg, 105 mg/kg, 110 mg/kg, 115 mg/kg, 120 mg/kg, 125 mg/kg, 130 mg/kg, 135 mg/kg, 140 mg/kg, 145 mg/kg, 150 mg/kg, 155 mg/kg, 160 mg/kg, 165 mg/kg, 170 mg/kg, 175 mg/kg, 180 mg/kg, 185 mg/kg, 190 mg/kg, 195 mg/kg, 200 mg/kg, and any fraction or integer in between any two of the preceding amounts.

In some embodiments, a NOS inhibitor composition comprising ARL-17477 and/or a salt thereof, such as, for example, ARL-17477 dihydrochloride, may be administered as a suitable dosage form, as described herein, to treat, ameliorate, and/or reduce the severity of NO-lung cancer or one or more symptoms thereof in an NO-lung cancer subject. In certain embodiments, an nNOS inhibitor composition comprising ARL-17477 and/or a salt thereof, such as, for example, ARL-17477 dihydrochloride, may be administered to an NO-lung cancer subject to treat, ameliorate, and/or reduce the severity of NO-lung cancer or one or more symptoms thereof in the NO-lung cancer subject: (i) via a route selected from the group consisting of buccally, inhalation, intraarterially, intraarticularly, intracardially, intracerebroventricularly, intradermally, intramedullary, intramuscularly, intraperitoneally, intrathecally, intravenously, intraventricularly, nasally, ophthalmically, orally, rectally, sublabially, sublingually, subcutaneously, topically, and transdermally; and/or (ii) with a frequency selected from the group consisting of four times daily, three times daily, twice daily, once daily, once every two days, once every three days, once every four days, once every five days, once every six days, once a week, once every eight days, once every nine days, once every ten days, once every two weeks, once every three weeks, and once a month; and/or (iii) at a dosage (based on subject weight) selected from the group consisting of 0.1 μg/kg, 0.5 μg/kg, 1 μg/kg, 1.5 μg/kg, 2.0 μg/kg, 2.5 μg/kg, 3.0 μg/kg, 3.5 μg/kg, 4.0 μg/kg, 4.5 μg/kg, 5.0 μg/kg, 10 μg/kg, 15 μg/kg, 20 μg/kg, 25 μg/kg, 30 μg/kg, 35 μg/kg, 40 μg/kg, 45 μg/kg, 50 μg/kg, 55 μg/kg, 60 μg/kg, 65 μg/kg, 70 μg/kg, 75 μg/kg, 80 μg/kg, 85 μg/kg, 90 μg/kg, 95 μg/kg, 100 μg/kg, 150 μg/kg, 200 μg/kg, 250 μg/kg, 300 μg/kg, 350 μg/kg, 400 μg/kg, 450 μg/kg, 500 μg/kg, 550 μg/kg, 600 μg/kg, 650 μg/kg, 700 μg/kg, 750 μg/kg, 800 μg/kg, 850 μg/kg, 900 μg/kg, 1 mg/kg, 1.5 mg/kg, 2.0 mg/kg, 2.5 mg/kg, 3 mg/kg, 3.5 mg/kg, 4.0 mg/kg, 4.5 mg/kg, 5 mg/kg, 5.5 mg/kg, 6 mg/kg, 6.5 mg/kg, 7 mg/kg, 7.5 mg/kg, 8 mg/kg, 8.5 mg/kg, 9 mg/kg, 9.5 mg/kg, 10 mg/kg 10.5 mg/kg, 11 mg/kg, 11.5 mg/kg, 12 mg/kg, 12.5 mg/kg, 13 mg/kg, 13.5 mg/kg, 14 mg/kg, 14.5 mg/kg, 15 mg/kg, 16 mg/kg, 17 mg/kg, 18 mg/kg, 19 mg/kg, 20 mg/kg, 21 mg/kg, 22 mg/kg, 23 mg/kg, 24 mg/kg, 25 mg/kg, 30 mg/kg, 35 mg/kg, 40 mg/kg, 45 mg/kg, 50 mg/kg, 55 mg/kg, 60 mg/kg, 65 mg/kg, 70 mg/kg, 75 mg/kg, 80 mg/kg, 85 mg/kg, 90 mg/kg, 95 mg/kg, 100 mg/kg, 105 mg/kg, 110 mg/kg, 115 mg/kg, 120 mg/kg, 125 mg/kg, 130 mg/kg, 135 mg/kg, 140 mg/kg, 145 mg/kg, 150 mg/kg, 155 mg/kg, 160 mg/kg, 165 mg/kg, 170 mg/kg, 175 mg/kg, 180 mg/kg, 185 mg/kg, 190 mg/kg, 195 mg/kg, 200 mg/kg, and any fraction or integer in between any two of the preceding amounts.

In certain embodiments, a NOS inhibitor composition comprising L-NNA and/or a salt thereof may be administered as a suitable dosage form, as described herein, to treat, ameliorate, and/or reduce the severity of NO-lung cancer or one or more symptoms thereof in an NO-lung cancer subject. In some embodiments, an nNOS inhibitor composition comprising L-NNA and/or a salt thereof may be administered to an NO-lung cancer subject to treat, ameliorate, and/or reduce the severity of NO-lung cancer or one or more symptoms thereof in the NO-lung cancer subject: (i) via a route selected from the group consisting of buccally, inhalation, intraarterially, intraarticularly, intracardially, intracerebroventricularly, intradermally, intramedullary, intramuscularly, intraperitoneally, intrathecally, intravenously, intraventricularly, nasally, ophthalmically, orally, rectally, sublabially, sublingually, subcutaneously, topically, and transdermally; and/or (ii) with a frequency selected from the group consisting of four times daily, three times daily, twice daily, once daily, once every two days, once every three days, once every four days, once every five days, once every six days, once a week, once every eight days, once every nine days, once every ten days, once every two weeks, once every three weeks, and once a month; and/or (iii) at a dosage (based on subject weight) selected from the group consisting of 0.1 μg/kg, 0.5 μg/kg, 1 μg/kg, 1.5 μg/kg, 2.0 μg/kg, 2.5 μg/kg, 3.0 μg/kg, 3.5 μg/kg, 4.0 μg/kg, 4.5 μg/kg, 5.0 μg/kg, 10 μg/kg, 15 μg/kg, 20 μg/kg, 25 μg/kg, 30 μg/kg, 35 μg/kg, 40 μg/kg, 45 μg/kg, 50 μg/kg, 55 μg/kg, 60 μg/kg, 65 μg/kg, 70 μg/kg, 75 μg/kg, 80 μg/kg, 85 μg/kg, 90 μg/kg, 95 μg/kg, 100 μg/kg, 150 μg/kg, 200 μg/kg, 250 μg/kg, 300 μg/kg, 350 μg/kg, 400 μg/kg, 450 μg/kg, 500 μg/kg, 550 μg/kg, 600 μg/kg, 650 μg/kg, 700 μg/kg, 750 μg/kg, 800 μg/kg, 850 μg/kg, 900 μg/kg, 1 mg/kg, 1.5 mg/kg, 2.0 mg/kg, 2.5 mg/kg, 3 mg/kg, 3.5 mg/kg, 4.0 mg/kg, 4.5 mg/kg, 5 mg/kg, 5.5 mg/kg, 6 mg/kg, 6.5 mg/kg, 7 mg/kg, 7.5 mg/kg, 8 mg/kg, 8.5 mg/kg, 9 mg/kg, 9.5 mg/kg, 10 mg/kg 10.5 mg/kg, 11 mg/kg, 11.5 mg/kg, 12 mg/kg, 12.5 mg/kg, 13 mg/kg, 13.5 mg/kg, 14 mg/kg, 14.5 mg/kg, 15 mg/kg, 16 mg/kg, 17 mg/kg, 18 mg/kg, 19 mg/kg, 20 mg/kg, 21 mg/kg, 22 mg/kg, 23 mg/kg, 24 mg/kg, 25 mg/kg, 30 mg/kg, 35 mg/kg, 40 mg/kg, 45 mg/kg, 50 mg/kg, 55 mg/kg, 60 mg/kg, 65 mg/kg, 70 mg/kg, 75 mg/kg, 80 mg/kg, 85 mg/kg, 90 mg/kg, 95 mg/kg, 100 mg/kg, 105 mg/kg, 110 mg/kg, 115 mg/kg, 120 mg/kg, 125 mg/kg, 130 mg/kg, 135 mg/kg, 140 mg/kg, 145 mg/kg, 150 mg/kg, 155 mg/kg, 160 mg/kg, 165 mg/kg, 170 mg/kg, 175 mg/kg, 180 mg/kg, 185 mg/kg, 190 mg/kg, 195 mg/kg, 200 mg/kg, and any fraction or integer in between any two of the preceding amounts.

In some embodiments, a NOS inhibitor composition comprising L-NAME and/or a salt thereof, such as, for example, L-NAME hydrochloride, may be administered as a suitable dosage form, as described herein, to treat, ameliorate, and/or reduce the severity of NO-lung cancer or one or more symptoms thereof in an NO-lung cancer subject. In certain embodiments, an nNOS inhibitor composition comprising L-NAME and/or a salt thereof, such as, for example, L-NAME hydrochloride, may be administered to an NO-lung cancer subject to treat, ameliorate, and/or reduce the severity of NO-lung cancer or one or more symptoms thereof in the NO-lung cancer subject: (i) via a route selected from the group consisting of buccally, inhalation, intraarterially, intraarticularly, intracardially, intracerebroventricularly, intradermally, intramedullary, intramuscularly, intraperitoneally, intrathecally, intravenously, intraventricularly, nasally, ophthalmically, orally, rectally, sublabially, sublingually, subcutaneously, topically, and transdermally; and/or (ii) with a frequency selected from the group consisting of four times daily, three times daily, twice daily, once daily, once every two days, once every three days, once every four days, once every five days, once every six days, once a week, once every eight days, once every nine days, once every ten days, once every two weeks, once every three weeks, and once a month; and/or (iii) at a dosage (based on subject weight) selected from the group consisting of 0.1 μg/kg, 0.5 μg/kg, 1 μg/kg, 1.5 μg/kg, 2.0 μg/kg, 2.5 μg/kg, 3.0 μg/kg, 3.5 μg/kg, 4.0 μg/kg, 4.5 μg/kg, 5.0 μg/kg, 10 μg/kg, 15 μg/kg, 20 μg/kg, 25 μg/kg, 30 μg/kg, 35 μg/kg, 40 μg/kg, 45 μg/kg, 50 μg/kg, 55 μg/kg, 60 μg/kg, 65 μg/kg, 70 μg/kg, 75 μg/kg, 80 μg/kg, 85 μg/kg, 90 μg/kg, 95 μg/kg, 100 μg/kg, 150 μg/kg, 200 μg/kg, 250 μg/kg, 300 μg/kg, 350 μg/kg, 400 μg/kg, 450 μg/kg, 500 μg/kg, 550 μg/kg, 600 μg/kg, 650 μg/kg, 700 μg/kg, 750 μg/kg, 800 μg/kg, 850 μg/kg, 900 μg/kg, 1 mg/kg, 1.5 mg/kg, 2.0 mg/kg, 2.5 mg/kg, 3 mg/kg, 3.5 mg/kg, 4.0 mg/kg, 4.5 mg/kg, 5 mg/kg, 5.5 mg/kg, 6 mg/kg, 6.5 mg/kg, 7 mg/kg, 7.5 mg/kg, 8 mg/kg, 8.5 mg/kg, 9 mg/kg, 9.5 mg/kg, 10 mg/kg 10.5 mg/kg, 11 mg/kg, 11.5 mg/kg, 12 mg/kg, 12.5 mg/kg, 13 mg/kg, 13.5 mg/kg, 14 mg/kg, 14.5 mg/kg, 15 mg/kg, 16 mg/kg, 17 mg/kg, 18 mg/kg, 19 mg/kg, 20 mg/kg, 21 mg/kg, 22 mg/kg, 23 mg/kg, 24 mg/kg, 25 mg/kg, 30 mg/kg, 35 mg/kg, 40 mg/kg, 45 mg/kg, 50 mg/kg, 55 mg/kg, 60 mg/kg, 65 mg/kg, 70 mg/kg, 75 mg/kg, 80 mg/kg, 85 mg/kg, 90 mg/kg, 95 mg/kg, 100 mg/kg, 105 mg/kg, 110 mg/kg, 115 mg/kg, 120 mg/kg, 125 mg/kg, 130 mg/kg, 135 mg/kg, 140 mg/kg, 145 mg/kg, 150 mg/kg, 155 mg/kg, 160 mg/kg, 165 mg/kg, 170 mg/kg, 175 mg/kg, 180 mg/kg, 185 mg/kg, 190 mg/kg, 195 mg/kg, 200 mg/kg, and any fraction or integer in between any two of the preceding amounts.

In certain embodiments, a NOS inhibitor composition comprising L-NMMA and/or a salt thereof, such as, for example, L-NMMA acetate, may be administered as a suitable dosage form, as described herein, to treat, ameliorate, and/or reduce the severity of NO-lung cancer or one or more symptoms thereof in an NO-lung cancer subject. In some embodiments, an nNOS inhibitor composition comprising L-NMMA and/or a salt thereof, such as, for example, L-NMMA acetate, may be administered to an NO-lung cancer subject to treat, ameliorate, and/or reduce the severity of NO-lung cancer or one or more symptoms thereof in the NO-lung cancer subject: (i) via a route selected from the group consisting of buccally, inhalation, intraarterially, intraarticularly, intracardially, intracerebroventricularly, intradermally, intramedullary, intramuscularly, intraperitoneally, intrathecally, intravenously, intraventricularly, nasally, ophthalmically, orally, rectally, sublabially, sublingually, subcutaneously, topically, and transdermally; and/or (ii) with a frequency selected from the group consisting of four times daily, three times daily, twice daily, once daily, once every two days, once every three days, once every four days, once every five days, once every six days, once a week, once every eight days, once every nine days, once every ten days, once every two weeks, once every three weeks, and once a month; and/or (iii) at a dosage (based on subject weight) selected from the group consisting of 0.1 μg/kg, 0.5 μg/kg, 1 μg/kg, 1.5 μg/kg, 2.0 μg/kg, 2.5 μg/kg, 3.0 μg/kg, 3.5 μg/kg, 4.0 μg/kg, 4.5 μg/kg, 5.0 μg/kg, 10 μg/kg, 15 μg/kg, 20 μg/kg, 25 μg/kg, 30 μg/kg, 35 μg/kg, 40 μg/kg, 45 μg/kg, 50 μg/kg, 55 μg/kg, 60 μg/kg, 65 μg/kg, 70 μg/kg, 75 μg/kg, 80 μg/kg, 85 μg/kg, 90 μg/kg, 95 μg/kg, 100 μg/kg, 150 μg/kg, 200 μg/kg, 250 μg/kg, 300 μg/kg, 350 μg/kg, 400 μg/kg, 450 μg/kg, 500 μg/kg, 550 μg/kg, 600 μg/kg, 650 μg/kg, 700 μg/kg, 750 μg/kg, 800 μg/kg, 850 μg/kg, 900 μg/kg, 1 mg/kg, 1.5 mg/kg, 2.0 mg/kg, 2.5 mg/kg, 3 mg/kg, 3.5 mg/kg, 4.0 mg/kg, 4.5 mg/kg, 5 mg/kg, 5.5 mg/kg, 6 mg/kg, 6.5 mg/kg, 7 mg/kg, 7.5 mg/kg, 8 mg/kg, 8.5 mg/kg, 9 mg/kg, 9.5 mg/kg, 10 mg/kg 10.5 mg/kg, 11 mg/kg, 11.5 mg/kg, 12 mg/kg, 12.5 mg/kg, 13 mg/kg, 13.5 mg/kg, 14 mg/kg, 14.5 mg/kg, 15 mg/kg, 16 mg/kg, 17 mg/kg, 18 mg/kg, 19 mg/kg, 20 mg/kg, 21 mg/kg, 22 mg/kg, 23 mg/kg, 24 mg/kg, 25 mg/kg, 30 mg/kg, 35 mg/kg, 40 mg/kg, 45 mg/kg, 50 mg/kg, 55 mg/kg, 60 mg/kg, 65 mg/kg, 70 mg/kg, 75 mg/kg, 80 mg/kg, 85 mg/kg, 90 mg/kg, 95 mg/kg, 100 mg/kg, 105 mg/kg, 110 mg/kg, 115 mg/kg, 120 mg/kg, 125 mg/kg, 130 mg/kg, 135 mg/kg, 140 mg/kg, 145 mg/kg, 150 mg/kg, 155 mg/kg, 160 mg/kg, 165 mg/kg, 170 mg/kg, 175 mg/kg, 180 mg/kg, 185 mg/kg, 190 mg/kg, 195 mg/kg, 200 mg/kg, and any fraction or integer in between any two of the preceding amounts.

In some embodiments, a NOS inhibitor composition comprising GW-274150 and/or a salt thereof, such as, for example, GW-274150 phosphate, may be administered as a suitable dosage form, as described herein, to treat, ameliorate, and/or reduce the severity of NO-lung cancer or one or more symptoms thereof in an NO-lung cancer subject. In certain embodiments, an nNOS inhibitor composition comprising GW-274150 and/or a salt thereof, such as, for example, GW-274150 phosphate, may be administered to an NO-lung cancer subject to treat, ameliorate, and/or reduce the severity of NO-lung cancer or one or more symptoms thereof in the NO-lung cancer subject: (i) via a route selected from the group consisting of buccally, inhalation, intraarterially, intraarticularly, intracardially, intracerebroventricularly, intradermally, intramedullary, intramuscularly, intraperitoneally, intrathecally, intravenously, intraventricularly, nasally, ophthalmically, orally, rectally, sublabially, sublingually, subcutaneously, topically, and transdermally; and/or (ii) with a frequency selected from the group consisting of four times daily, three times daily, twice daily, once daily, once every two days, once every three days, once every four days, once every five days, once every six days, once a week, once every eight days, once every nine days, once every ten days, once every two weeks, once every three weeks, and once a month; and/or (iii) at a dosage (based on subject weight) selected from the group consisting of 0.1 μg/kg, 0.5 μg/kg, 1 μg/kg, 1.5 μg/kg, 2.0 μg/kg, 2.5 μg/kg, 3.0 μg/kg, 3.5 μg/kg, 4.0 μg/kg, 4.5 μg/kg, 5.0 μg/kg, 10 μg/kg, 15 μg/kg, 20 μg/kg, 25 μg/kg, 30 μg/kg, 35 μg/kg, 40 μg/kg, 45 μg/kg, 50 μg/kg, 55 μg/kg, 60 μg/kg, 65 μg/kg, 70 μg/kg, 75 μg/kg, 80 μg/kg, 85 μg/kg, 90 μg/kg, 95 μg/kg, 100 μg/kg, 150 μg/kg, 200 μg/kg, 250 μg/kg, 300 μg/kg, 350 μg/kg, 400 μg/kg, 450 μg/kg, 500 μg/kg, 550 μg/kg, 600 μg/kg, 650 μg/kg, 700 μg/kg, 750 μg/kg, 800 μg/kg, 850 μg/kg, 900 μg/kg, 1 mg/kg, 1.5 mg/kg, 2.0 mg/kg, 2.5 mg/kg, 3 mg/kg, 3.5 mg/kg, 4.0 mg/kg, 4.5 mg/kg, 5 mg/kg, 5.5 mg/kg, 6 mg/kg, 6.5 mg/kg, 7 mg/kg, 7.5 mg/kg, 8 mg/kg, 8.5 mg/kg, 9 mg/kg, 9.5 mg/kg, 10 mg/kg 10.5 mg/kg, 11 mg/kg, 11.5 mg/kg, 12 mg/kg, 12.5 mg/kg, 13 mg/kg, 13.5 mg/kg, 14 mg/kg, 14.5 mg/kg, 15 mg/kg, 16 mg/kg, 17 mg/kg, 18 mg/kg, 19 mg/kg, 20 mg/kg, 21 mg/kg, 22 mg/kg, 23 mg/kg, 24 mg/kg, 25 mg/kg, 30 mg/kg, 35 mg/kg, 40 mg/kg, 45 mg/kg, 50 mg/kg, 55 mg/kg, 60 mg/kg, 65 mg/kg, 70 mg/kg, 75 mg/kg, 80 mg/kg, 85 mg/kg, 90 mg/kg, 95 mg/kg, 100 mg/kg, 105 mg/kg, 110 mg/kg, 115 mg/kg, 120 mg/kg, 125 mg/kg, 130 mg/kg, 135 mg/kg, 140 mg/kg, 145 mg/kg, 150 mg/kg, 155 mg/kg, 160 mg/kg, 165 mg/kg, 170 mg/kg, 175 mg/kg, 180 mg/kg, 185 mg/kg, 190 mg/kg, 195 mg/kg, 200 mg/kg, and any fraction or integer in between any two of the preceding amounts.

In certain embodiments, a NOS inhibitor composition comprising L-NIL and/or a salt thereof, such as, for example, L-NIL hydrochloride, may be administered as a suitable dosage form, as described herein, to treat, ameliorate, and/or reduce the severity of NO-lung cancer or one or more symptoms thereof in an NO-lung cancer subject. In some embodiments, an nNOS inhibitor composition comprising L-NIL and/or a salt thereof, such as, for example, L-NIL hydrochloride, may be administered to an NO-lung cancer subject to treat, ameliorate, and/or reduce the severity of NO-lung cancer or one or more symptoms thereof in the NO-lung cancer subject: (i) via a route selected from the group consisting of buccally, inhalation, intraarterially, intraarticularly, intracardially, intracerebroventricularly, intradermally, intramedullary, intramuscularly, intraperitoneally, intrathecally, intravenously, intraventricularly, nasally, ophthalmically, orally, rectally, sublabially, sublingually, subcutaneously, topically, and transdermally; and/or (ii) with a frequency selected from the group consisting of four times daily, three times daily, twice daily, once daily, once every two days, once every three days, once every four days, once every five days, once every six days, once a week, once every eight days, once every nine days, once every ten days, once every two weeks, once every three weeks, and once a month; and/or (iii) at a dosage (based on subject weight) selected from the group consisting of 0.1 μg/kg, 0.5 μg/kg, 1 μg/kg, 1.5 μg/kg, 2.0 μg/kg, 2.5 μg/kg, 3.0 μg/kg, 3.5 μg/kg, 4.0 μg/kg, 4.5 μg/kg, 5.0 μg/kg, 10 μg/kg, 15 μg/kg, 20 μg/kg, 25 μg/kg, 30 μg/kg, 35 μg/kg, 40 μg/kg, 45 μg/kg, 50 μg/kg, 55 μg/kg, 60 μg/kg, 65 μg/kg, 70 μg/kg, 75 μg/kg, 80 μg/kg, 85 μg/kg, 90 μg/kg, 95 μg/kg, 100 μg/kg, 150 μg/kg, 200 μg/kg, 250 μg/kg, 300 μg/kg, 350 μg/kg, 400 μg/kg, 450 μg/kg, 500 μg/kg, 550 μg/kg, 600 μg/kg, 650 μg/kg, 700 μg/kg, 750 μg/kg, 800 μg/kg, 850 μg/kg, 900 μg/kg, 1 mg/kg, 1.5 mg/kg, 2.0 mg/kg, 2.5 mg/kg, 3 mg/kg, 3.5 mg/kg, 4.0 mg/kg, 4.5 mg/kg, 5 mg/kg, 5.5 mg/kg, 6 mg/kg, 6.5 mg/kg, 7 mg/kg, 7.5 mg/kg, 8 mg/kg, 8.5 mg/kg, 9 mg/kg, 9.5 mg/kg, 10 mg/kg 10.5 mg/kg, 11 mg/kg, 11.5 mg/kg, 12 mg/kg, 12.5 mg/kg, 13 mg/kg, 13.5 mg/kg, 14 mg/kg, 14.5 mg/kg, 15 mg/kg, 16 mg/kg, 17 mg/kg, 18 mg/kg, 19 mg/kg, 20 mg/kg, 21 mg/kg, 22 mg/kg, 23 mg/kg, 24 mg/kg, 25 mg/kg, 30 mg/kg, 35 mg/kg, 40 mg/kg, 45 mg/kg, 50 mg/kg, 55 mg/kg, 60 mg/kg, 65 mg/kg, 70 mg/kg, 75 mg/kg, 80 mg/kg, 85 mg/kg, 90 mg/kg, 95 mg/kg, 100 mg/kg, 105 mg/kg, 110 mg/kg, 115 mg/kg, 120 mg/kg, 125 mg/kg, 130 mg/kg, 135 mg/kg, 140 mg/kg, 145 mg/kg, 150 mg/kg, 155 mg/kg, 160 mg/kg, 165 mg/kg, 170 mg/kg, 175 mg/kg, 180 mg/kg, 185 mg/kg, 190 mg/kg, 195 mg/kg, 200 mg/kg, and any fraction or integer in between any two of the preceding amounts.

In some embodiments, a NOS inhibitor composition comprising 1400W and/or a salt thereof, such as, for example, 1400W dihydrochloride, may be administered as a suitable dosage form, as described herein, to treat, ameliorate, and/or reduce the severity of NO-lung cancer or one or more symptoms thereof in an NO-lung cancer subject. In certain embodiments, an nNOS inhibitor composition comprising 1400W and/or a salt thereof, such as, for example, 1400W dihydrochloride, may be administered to an NO-lung cancer subject to treat, ameliorate, and/or reduce the severity of NO-lung cancer or one or more symptoms thereof in the NO-lung cancer subject: (i) via a route selected from the group consisting of buccally, inhalation, intraarterially, intraarticularly, intracardially, intracerebroventricularly, intradermally, intramedullary, intramuscularly, intraperitoneally, intrathecally, intravenously, intraventricularly, nasally, ophthalmically, orally, rectally, sublabially, sublingually, subcutaneously, topically, and transdermally; and/or (ii) with a frequency selected from the group consisting of four times daily, three times daily, twice daily, once daily, once every two days, once every three days, once every four days, once every five days, once every six days, once a week, once every eight days, once every nine days, once every ten days, once every two weeks, once every three weeks, and once a month; and/or (iii) at a dosage (based on subject weight) selected from the group consisting of 0.1 μg/kg, 0.5 μg/kg, 1 μg/kg, 1.5 μg/kg, 2.0 μg/kg, 2.5 μg/kg, 3.0 μg/kg, 3.5 μg/kg, 4.0 μg/kg, 4.5 μg/kg, 5.0 μg/kg, 10 μg/kg, 15 μg/kg, 20 μg/kg, 25 μg/kg, 30 μg/kg, 35 μg/kg, 40 μg/kg, 45 μg/kg, 50 μg/kg, 55 μg/kg, 60 μg/kg, 65 μg/kg, 70 μg/kg, 75 μg/kg, 80 μg/kg, 85 μg/kg, 90 μg/kg, 95 μg/kg, 100 μg/kg, 150 μg/kg, 200 μg/kg, 250 μg/kg, 300 μg/kg, 350 μg/kg, 400 μg/kg, 450 μg/kg, 500 μg/kg, 550 μg/kg, 600 μg/kg, 650 μg/kg, 700 μg/kg, 750 μg/kg, 800 μg/kg, 850 μg/kg, 900 μg/kg, 1 mg/kg, 1.5 mg/kg, 2.0 mg/kg, 2.5 mg/kg, 3 mg/kg, 3.5 mg/kg, 4.0 mg/kg, 4.5 mg/kg, 5 mg/kg, 5.5 mg/kg, 6 mg/kg, 6.5 mg/kg, 7 mg/kg, 7.5 mg/kg, 8 mg/kg, 8.5 mg/kg, 9 mg/kg, 9.5 mg/kg, 10 mg/kg 10.5 mg/kg, 11 mg/kg, 11.5 mg/kg, 12 mg/kg, 12.5 mg/kg, 13 mg/kg, 13.5 mg/kg, 14 mg/kg, 14.5 mg/kg, 15 mg/kg, 16 mg/kg, 17 mg/kg, 18 mg/kg, 19 mg/kg, 20 mg/kg, 21 mg/kg, 22 mg/kg, 23 mg/kg, 24 mg/kg, 25 mg/kg, 30 mg/kg, 35 mg/kg, 40 mg/kg, 45 mg/kg, 50 mg/kg, 55 mg/kg, 60 mg/kg, 65 mg/kg, 70 mg/kg, 75 mg/kg, 80 mg/kg, 85 mg/kg, 90 mg/kg, 95 mg/kg, 100 mg/kg, 105 mg/kg, 110 mg/kg, 115 mg/kg, 120 mg/kg, 125 mg/kg, 130 mg/kg, 135 mg/kg, 140 mg/kg, 145 mg/kg, 150 mg/kg, 155 mg/kg, 160 mg/kg, 165 mg/kg, 170 mg/kg, 175 mg/kg, 180 mg/kg, 185 mg/kg, 190 mg/kg, 195 mg/kg, 200 mg/kg, and any fraction or integer in between any two of the preceding amounts.

In certain embodiments, a NOS inhibitor composition comprising OKN-007 and/or a salt thereof, such as, for example, OKN-007 sodium, may be administered as a suitable dosage form, as described herein, to treat, ameliorate, and/or reduce the severity of NO-lung cancer or one or more symptoms thereof in an NO-lung cancer subject. In some embodiments, an nNOS inhibitor composition comprising OKN-007 and/or a salt thereof, such as, for example, OKN-007 sodium, may be administered to an NO-lung cancer subject to treat, ameliorate, and/or reduce the severity of NO-lung cancer or one or more symptoms thereof in the NO-lung cancer subject: (i) via a route selected from the group consisting of buccally, inhalation, intraarterially, intraarticularly, intracardially, intracerebroventricularly, intradermally, intramedullary, intramuscularly, intraperitoneally, intrathecally, intravenously, intraventricularly, nasally, ophthalmically, orally, rectally, sublabially, sublingually, subcutaneously, topically, and transdermally; and/or (ii) with a frequency selected from the group consisting of four times daily, three times daily, twice daily, once daily, once every two days, once every three days, once every four days, once every five days, once every six days, once a week, once every eight days, once every nine days, once every ten days, once every two weeks, once every three weeks, and once a month; and/or (iii) at a dosage (based on subject weight) selected from the group consisting of 0.1 μg/kg, 0.5 μg/kg, 1 μg/kg, 1.5 μg/kg, 2.0 μg/kg, 2.5 μg/kg, 3.0 μg/kg, 3.5 μg/kg, 4.0 μg/kg, 4.5 μg/kg, 5.0 μg/kg, 10 μg/kg, 15 μg/kg, 20 μg/kg, 25 μg/kg, 30 μg/kg, 35 μg/kg, 40 μg/kg, 45 μg/kg, 50 μg/kg, 55 μg/kg, 60 μg/kg, 65 μg/kg, 70 μg/kg, 75 μg/kg, 80 μg/kg, 85 μg/kg, 90 μg/kg, 95 μg/kg, 100 μg/kg, 150 μg/kg, 200 μg/kg, 250 μg/kg, 300 μg/kg, 350 μg/kg, 400 μg/kg, 450 μg/kg, 500 μg/kg, 550 μg/kg, 600 μg/kg, 650 μg/kg, 700 μg/kg, 750 μg/kg, 800 μg/kg, 850 μg/kg, 900 μg/kg, 1 mg/kg, 1.5 mg/kg, 2.0 mg/kg, 2.5 mg/kg, 3 mg/kg, 3.5 mg/kg, 4.0 mg/kg, 4.5 mg/kg, 5 mg/kg, 5.5 mg/kg, 6 mg/kg, 6.5 mg/kg, 7 mg/kg, 7.5 mg/kg, 8 mg/kg, 8.5 mg/kg, 9 mg/kg, 9.5 mg/kg, 10 mg/kg 10.5 mg/kg, 11 mg/kg, 11.5 mg/kg, 12 mg/kg, 12.5 mg/kg, 13 mg/kg, 13.5 mg/kg, 14 mg/kg, 14.5 mg/kg, 15 mg/kg, 16 mg/kg, 17 mg/kg, 18 mg/kg, 19 mg/kg, 20 mg/kg, 21 mg/kg, 22 mg/kg, 23 mg/kg, 24 mg/kg, 25 mg/kg, 30 mg/kg, 35 mg/kg, 40 mg/kg, 45 mg/kg, 50 mg/kg, 55 mg/kg, 60 mg/kg, 65 mg/kg, 70 mg/kg, 75 mg/kg, 80 mg/kg, 85 mg/kg, 90 mg/kg, 95 mg/kg, 100 mg/kg, 105 mg/kg, 110 mg/kg, 115 mg/kg, 120 mg/kg, 125 mg/kg, 130 mg/kg, 135 mg/kg, 140 mg/kg, 145 mg/kg, 150 mg/kg, 155 mg/kg, 160 mg/kg, 165 mg/kg, 170 mg/kg, 175 mg/kg, 180 mg/kg, 185 mg/kg, 190 mg/kg, 195 mg/kg, 200 mg/kg, and any fraction or integer in between any two of the preceding amounts.

In some embodiments, a NOS inhibitor composition comprising BYK-191023 and/or a salt thereof, such as, for example, BYK-191023 dihydrochloride, may be administered as a suitable dosage form, as described herein, to treat, ameliorate, and/or reduce the severity of NO-lung cancer or one or more symptoms thereof in an NO-lung cancer subject. In certain embodiments, an nNOS inhibitor composition comprising BYK-191023 and/or a salt thereof, such as, for example, BYK-191023 dihydrochloride, may be administered to an NO-lung cancer subject to treat, ameliorate, and/or reduce the severity of NO-lung cancer or one or more symptoms thereof in the NO-lung cancer subject: (i) via a route selected from the group consisting of buccally, inhalation, intraarterially, intraarticularly, intracardially, intracerebroventricularly, intradermally, intramedullary, intramuscularly, intraperitoneally, intrathecally, intravenously, intraventricularly, nasally, ophthalmically, orally, rectally, sublabially, sublingually, subcutaneously, topically, and transdermally; and/or (ii) with a frequency selected from the group consisting of four times daily, three times daily, twice daily, once daily, once every two days, once every three days, once every four days, once every five days, once every six days, once a week, once every eight days, once every nine days, once every ten days, once every two weeks, once every three weeks, and once a month; and/or (iii) at a dosage (based on subject weight) selected from the group consisting of 0.1 μg/kg, 0.5 μg/kg, 1 μg/kg, 1.5 μg/kg, 2.0 μg/kg, 2.5 μg/kg, 3.0 μg/kg, 3.5 μg/kg, 4.0 μg/kg, 4.5 μg/kg, 5.0 μg/kg, 10 μg/kg, 15 μg/kg, 20 μg/kg, 25 μg/kg, 30 μg/kg, 35 μg/kg, 40 μg/kg, 45 μg/kg, 50 μg/kg, 55 μg/kg, 60 μg/kg, 65 μg/kg, 70 μg/kg, 75 μg/kg, 80 μg/kg, 85 μg/kg, 90 μg/kg, 95 μg/kg, 100 μg/kg, 150 μg/kg, 200 μg/kg, 250 μg/kg, 300 μg/kg, 350 μg/kg, 400 μg/kg, 450 μg/kg, 500 μg/kg, 550 μg/kg, 600 μg/kg, 650 μg/kg, 700 μg/kg, 750 μg/kg, 800 μg/kg, 850 μg/kg, 900 μg/kg, 1 mg/kg, 1.5 mg/kg, 2.0 mg/kg, 2.5 mg/kg, 3 mg/kg, 3.5 mg/kg, 4.0 mg/kg, 4.5 mg/kg, 5 mg/kg, 5.5 mg/kg, 6 mg/kg, 6.5 mg/kg, 7 mg/kg, 7.5 mg/kg, 8 mg/kg, 8.5 mg/kg, 9 mg/kg, 9.5 mg/kg, 10 mg/kg 10.5 mg/kg, 11 mg/kg, 11.5 mg/kg, 12 mg/kg, 12.5 mg/kg, 13 mg/kg, 13.5 mg/kg, 14 mg/kg, 14.5 mg/kg, 15 mg/kg, 16 mg/kg, 17 mg/kg, 18 mg/kg, 19 mg/kg, 20 mg/kg, 21 mg/kg, 22 mg/kg, 23 mg/kg, 24 mg/kg, 25 mg/kg, 30 mg/kg, 35 mg/kg, 40 mg/kg, 45 mg/kg, 50 mg/kg, 55 mg/kg, 60 mg/kg, 65 mg/kg, 70 mg/kg, 75 mg/kg, 80 mg/kg, 85 mg/kg, 90 mg/kg, 95 mg/kg, 100 mg/kg, 105 mg/kg, 110 mg/kg, 115 mg/kg, 120 mg/kg, 125 mg/kg, 130 mg/kg, 135 mg/kg, 140 mg/kg, 145 mg/kg, 150 mg/kg, 155 mg/kg, 160 mg/kg, 165 mg/kg, 170 mg/kg, 175 mg/kg, 180 mg/kg, 185 mg/kg, 190 mg/kg, 195 mg/kg, 200 mg/kg, and any fraction or integer in between any two of the preceding amounts.

In certain embodiments, a NOS inhibitor composition comprising CM544 and/or a salt thereof, such as, for example, CM544 dihydrochloride, may be administered as a suitable dosage form, as described herein, to treat, ameliorate, and/or reduce the severity of NO-lung cancer or one or more symptoms thereof in an NO-lung cancer subject. In some embodiments, an nNOS inhibitor composition comprising CM544 and/or a salt thereof, such as, for example, CM544 dihydrochloride, may be administered to an NO-lung cancer subject to treat, ameliorate, and/or reduce the severity of NO-lung cancer or one or more symptoms thereof in the NO-lung cancer subject: (i) via a route selected from the group consisting of buccally, inhalation, intraarterially, intraarticularly, intracardially, intracerebroventricularly, intradermally, intramedullary, intramuscularly, intraperitoneally, intrathecally, intravenously, intraventricularly, nasally, ophthalmically, orally, rectally, sublabially, sublingually, subcutaneously, topically, and transdermally; and/or (ii) with a frequency selected from the group consisting of four times daily, three times daily, twice daily, once daily, once every two days, once every three days, once every four days, once every five days, once every six days, once a week, once every eight days, once every nine days, once every ten days, once every two weeks, once every three weeks, and once a month; and/or (iii) at a dosage (based on subject weight) selected from the group consisting of 0.1 μg/kg, 0.5 μg/kg, 1 μg/kg, 1.5 μg/kg, 2.0 μg/kg, 2.5 μg/kg, 3.0 μg/kg, 3.5 μg/kg, 4.0 μg/kg, 4.5 μg/kg, 5.0 μg/kg, 10 μg/kg, 15 μg/kg, 20 μg/kg, 25 μg/kg, 30 μg/kg, 35 μg/kg, 40 μg/kg, 45 μg/kg, 50 μg/kg, 55 μg/kg, 60 μg/kg, 65 μg/kg, 70 μg/kg, 75 μg/kg, 80 μg/kg, 85 μg/kg, 90 μg/kg, 95 μg/kg, 100 μg/kg, 150 μg/kg, 200 μg/kg, 250 μg/kg, 300 μg/kg, 350 μg/kg, 400 μg/kg, 450 μg/kg, 500 μg/kg, 550 μg/kg, 600 μg/kg, 650 μg/kg, 700 μg/kg, 750 μg/kg, 800 μg/kg, 850 μg/kg, 900 μg/kg, 1 mg/kg, 1.5 mg/kg, 2.0 mg/kg, 2.5 mg/kg, 3 mg/kg, 3.5 mg/kg, 4.0 mg/kg, 4.5 mg/kg, 5 mg/kg, 5.5 mg/kg, 6 mg/kg, 6.5 mg/kg, 7 mg/kg, 7.5 mg/kg, 8 mg/kg, 8.5 mg/kg, 9 mg/kg, 9.5 mg/kg, 10 mg/kg 10.5 mg/kg, 11 mg/kg, 11.5 mg/kg, 12 mg/kg, 12.5 mg/kg, 13 mg/kg, 13.5 mg/kg, 14 mg/kg, 14.5 mg/kg, 15 mg/kg, 16 mg/kg, 17 mg/kg, 18 mg/kg, 19 mg/kg, 20 mg/kg, 21 mg/kg, 22 mg/kg, 23 mg/kg, 24 mg/kg, 25 mg/kg, 30 mg/kg, 35 mg/kg, 40 mg/kg, 45 mg/kg, 50 mg/kg, 55 mg/kg, 60 mg/kg, 65 mg/kg, 70 mg/kg, 75 mg/kg, 80 mg/kg, 85 mg/kg, 90 mg/kg, 95 mg/kg, 100 mg/kg, 105 mg/kg, 110 mg/kg, 115 mg/kg, 120 mg/kg, 125 mg/kg, 130 mg/kg, 135 mg/kg, 140 mg/kg, 145 mg/kg, 150 mg/kg, 155 mg/kg, 160 mg/kg, 165 mg/kg, 170 mg/kg, 175 mg/kg, 180 mg/kg, 185 mg/kg, 190 mg/kg, 195 mg/kg, 200 mg/kg, and any fraction or integer in between any two of the preceding amounts.

In certain embodiments, an nNOS inhibitor composition comprising one or more dsRNAs each comprising at least 30 base pairs, may be administered as a suitable dosage form, as described herein, to treat, ameliorate, and/or reduce the severity of NO-lung cancer or one or more symptoms thereof in an NO-lung cancer subject. In some embodiments, an nNOS inhibitor composition comprising one or more dsRNAs, each comprising at least 30 base pairs, may be administered to an NO-lung cancer subject to treat, ameliorate, and/or reduce the severity of NO-lung cancer or one or more symptoms thereof in the NO-lung cancer subject: (i) via a route selected from the group consisting of buccally, inhalation, intraarterially, intraarticularly, intracardially, intracerebroventricularly, intradermally, intramedullary, intramuscularly, intraperitoneally, intrathecally, intravenously, intraventricularly, nasally, ophthalmically, orally, rectally, sublabially, sublingually, subcutaneously, topically, and transdermally; and/or (ii) with a frequency selected from the group consisting of four times daily, three times daily, twice daily, once daily, once every two days, once every three days, once every four days, once every five days, once every six days, once a week, once every eight days, once every nine days, once every ten days, once every two weeks, once every three weeks, and once a month; and/or (iii) at a dosage (based on subject weight) selected from the group consisting of 0.1 μg/kg, 0.5 μg/kg, 1 μg/kg, 1.5 μg/kg, 2.0 μg/kg, 2.5 μg/kg, 3.0 μg/kg, 3.5 μg/kg, 4.0 μg/kg, 4.5 μg/kg, 5.0 μg/kg, 10 μg/kg, 15 μg/kg, 20 μg/kg, 25 μg/kg, 30 μg/kg, 35 μg/kg, 40 μg/kg, 45 μg/kg, 50 μg/kg, 55 μg/kg, 60 μg/kg, 65 μg/kg, 70 μg/kg, 75 μg/kg, 80 μg/kg, 85 μg/kg, 90 μg/kg, 95 μg/kg, 100 μg/kg, 150 μg/kg, 200 μg/kg, 250 μg/kg, 300 μg/kg, 350 μg/kg, 400 μg/kg, 450 μg/kg, 500 μg/kg, 550 μg/kg, 600 μg/kg, 650 μg/kg, 700 μg/kg, 750 μg/kg, 800 μg/kg, 850 μg/kg, 900 μg/kg, 1 mg/kg, 1.5 mg/kg, 2.0 mg/kg, 2.5 mg/kg, 3 mg/kg, 3.5 mg/kg, 4.0 mg/kg, 4.5 mg/kg, 5 mg/kg, 5.5 mg/kg, 6 mg/kg, 6.5 mg/kg, 7 mg/kg, 7.5 mg/kg, 8 mg/kg, 8.5 mg/kg, 9 mg/kg, 9.5 mg/kg, 10 mg/kg 10.5 mg/kg, 11 mg/kg, 11.5 mg/kg, 12 mg/kg, 12.5 mg/kg, 13 mg/kg, 13.5 mg/kg, 14 mg/kg, 14.5 mg/kg, 15 mg/kg, 16 mg/kg, 17 mg/kg, 18 mg/kg, 19 mg/kg, 20 mg/kg, 21 mg/kg, 22 mg/kg, 23 mg/kg, 24 mg/kg, 25 mg/kg, 30 mg/kg, 35 mg/kg, 40 mg/kg, 45 mg/kg, 50 mg/kg, 55 mg/kg, 60 mg/kg, 65 mg/kg, 70 mg/kg, 75 mg/kg, 80 mg/kg, 85 mg/kg, 90 mg/kg, 95 mg/kg, 100 mg/kg, 105 mg/kg, 110 mg/kg, 115 mg/kg, 120 mg/kg, 125 mg/kg, 130 mg/kg, 135 mg/kg, 140 mg/kg, 145 mg/kg, 150 mg/kg, 155 mg/kg, 160 mg/kg, 165 mg/kg, 170 mg/kg, 175 mg/kg, 180 mg/kg, 185 mg/kg, 190 mg/kg, 195 mg/kg, 200 mg/kg, and any fraction or integer in between any two of the preceding amounts.

In some embodiments, an nNOS inhibitor composition comprising one or more siRNAs, may be administered as a suitable dosage form, as described herein, to treat, ameliorate, and/or reduce the severity of NO-lung cancer or one or more symptoms thereof in an NO-lung cancer subject. In certain embodiments, an nNOS inhibitor composition comprising one or more siRNAs selected from the group consisting of SEQ ID NO. 4, SEQ ID NO. 5, SEQ ID NO. 6, SEQ ID NO. 7, SEQ ID NO. 8, and any combination thereof, may be administered as a suitable dosage form, as described herein, to treat, ameliorate, and/or reduce the severity of NO-lung cancer or one or more symptoms thereof in an NO-lung cancer subject. In some embodiments, an nNOS inhibitor composition comprising one or more siRNAs selected from the group consisting of SEQ ID NO. 4, SEQ ID NO. 5, SEQ ID NO. 6, SEQ ID NO. 7, SEQ ID NO. 8, and any combination thereof, may be administered to an NO-lung cancer subject to treat, ameliorate, and/or reduce the severity of NO-lung cancer or one or more symptoms thereof in the NO-lung cancer subject: (i) via a route selected from the group consisting of buccally, inhalation, intraarterially, intraarticularly, intracardially, intracerebroventricularly, intradermally, intramedullary, intramuscularly, intraperitoneally, intrathecally, intravenously, intraventricularly, nasally, ophthalmically, orally, rectally, sublabially, sublingually, subcutaneously, topically, and transdermally; and/or (ii) with a frequency selected from the group consisting of four times daily, three times daily, twice daily, once daily, once every two days, once every three days, once every four days, once every five days, once every six days, once a week, once every eight days, once every nine days, once every ten days, once every two weeks, once every three weeks, and once a month; and/or (iii) at a dosage (based on subject weight) selected from the group consisting of 0.1 μg/kg, 0.5 μg/kg, 1 μg/kg, 1.5 μg/kg, 2.0 μg/kg, 2.5 μg/kg, 3.0 μg/kg, 3.5 μg/kg, 4.0 μg/kg, 4.5 μg/kg, 5.0 μg/kg, 10 μg/kg, 15 μg/kg, 20 μg/kg, 25 μg/kg, 30 μg/kg, 35 μg/kg, 40 μg/kg, 45 μg/kg, 50 μg/kg, 55 μg/kg, 60 μg/kg, 65 μg/kg, 70 μg/kg, 75 μg/kg, 80 μg/kg, 85 μg/kg, 90 μg/kg, 95 μg/kg, 100 μg/kg, 150 μg/kg, 200 μg/kg, 250 μg/kg, 300 μg/kg, 350 μg/kg, 400 μg/kg, 450 μg/kg, 500 μg/kg, 550 μg/kg, 600 μg/kg, 650 μg/kg, 700 μg/kg, 750 μg/kg, 800 μg/kg, 850 μg/kg, 900 μg/kg, 1 mg/kg, 1.5 mg/kg, 2.0 mg/kg, 2.5 mg/kg, 3 mg/kg, 3.5 mg/kg, 4.0 mg/kg, 4.5 mg/kg, 5 mg/kg, 5.5 mg/kg, 6 mg/kg, 6.5 mg/kg, 7 mg/kg, 7.5 mg/kg, 8 mg/kg, 8.5 mg/kg, 9 mg/kg, 9.5 mg/kg, 10 mg/kg 10.5 mg/kg, 11 mg/kg, 11.5 mg/kg, 12 mg/kg, 12.5 mg/kg, 13 mg/kg, 13.5 mg/kg, 14 mg/kg, 14.5 mg/kg, 15 mg/kg, 16 mg/kg, 17 mg/kg, 18 mg/kg, 19 mg/kg, 20 mg/kg, 21 mg/kg, 22 mg/kg, 23 mg/kg, 24 mg/kg, 25 mg/kg, 30 mg/kg, 35 mg/kg, 40 mg/kg, 45 mg/kg, 50 mg/kg, 55 mg/kg, 60 mg/kg, 65 mg/kg, 70 mg/kg, 75 mg/kg, 80 mg/kg, 85 mg/kg, 90 mg/kg, 95 mg/kg, 100 mg/kg, 105 mg/kg, 110 mg/kg, 115 mg/kg, 120 mg/kg, 125 mg/kg, 130 mg/kg, 135 mg/kg, 140 mg/kg, 145 mg/kg, 150 mg/kg, 155 mg/kg, 160 mg/kg, 165 mg/kg, 170 mg/kg, 175 mg/kg, 180 mg/kg, 185 mg/kg, 190 mg/kg, 195 mg/kg, 200 mg/kg, and any fraction or integer in between any two of the preceding amounts.

In some embodiments, an nNOS inhibitor composition comprising one or more shRNAs, may be administered as a suitable dosage form, as described herein, to treat, ameliorate, and/or reduce the severity of NO-lung cancer or one or more symptoms thereof in an NO-lung cancer subject. In certain embodiments, an nNOS inhibitor composition comprising one or more shRNAs selected from the group consisting of SEQ ID NO. 9, SEQ ID NO. 10, SEQ ID NO. 11, SEQ ID NO. 12, SEQ ID NO. 13, SEQ ID NO. 14, and any combination thereof, may be administered as a suitable dosage form, as described herein, to treat, ameliorate, and/or reduce the severity of NO-lung cancer or one or more symptoms thereof in an NO-lung cancer subject. In some embodiments, an nNOS inhibitor composition comprising one or more shRNAs selected from the group consisting of SEQ ID NO. 9, SEQ ID NO. 10, SEQ ID NO. 11, SEQ ID NO. 12, SEQ ID NO. 13, SEQ ID NO. 14, and any combination thereof, may be administered to an NO-lung cancer subject to treat, ameliorate, and/or reduce the severity of NO-lung cancer or one or more symptoms thereof in the NO-lung cancer subject: (i) via a route selected from the group consisting of buccally, inhalation, intraarterially, intraarticularly, intracardially, intracerebroventricularly, intradermally, intramedullary, intramuscularly, intraperitoneally, intrathecally, intravenously, intraventricularly, nasally, ophthalmically, orally, rectally, sublabially, sublingually, subcutaneously, topically, and transdermally; and/or (ii) with a frequency selected from the group consisting of four times daily, three times daily, twice daily, once daily, once every two days, once every three days, once every four days, once every five days, once every six days, once a week, once every eight days, once every nine days, once every ten days, once every two weeks, once every three weeks, and once a month; and/or (iii) at a dosage (based on subject weight) selected from the group consisting of 0.1 μg/kg, 0.5 μg/kg, 1 μg/kg, 1.5 μg/kg, 2.0 μg/kg, 2.5 μg/kg, 3.0 μg/kg, 3.5 μg/kg, 4.0 μg/kg, 4.5 μg/kg, 5.0 μg/kg, 10 μg/kg, 15 μg/kg, 20 μg/kg, 25 μg/kg, 30 μg/kg, 35 μg/kg, 40 μg/kg, 45 μg/kg, 50 μg/kg, 55 μg/kg, 60 μg/kg, 65 μg/kg, 70 μg/kg, 75 μg/kg, 80 μg/kg, 85 μg/kg, 90 μg/kg, 95 μg/kg, 100 μg/kg, 150 μg/kg, 200 μg/kg, 250 μg/kg, 300 μg/kg, 350 μg/kg, 400 μg/kg, 450 μg/kg, 500 μg/kg, 550 μg/kg, 600 μg/kg, 650 μg/kg, 700 μg/kg, 750 μg/kg, 800 μg/kg, 850 μg/kg, 900 μg/kg, 1 mg/kg, 1.5 mg/kg, 2.0 mg/kg, 2.5 mg/kg, 3 mg/kg, 3.5 mg/kg, 4.0 mg/kg, 4.5 mg/kg, 5 mg/kg, 5.5 mg/kg, 6 mg/kg, 6.5 mg/kg, 7 mg/kg, 7.5 mg/kg, 8 mg/kg, 8.5 mg/kg, 9 mg/kg, 9.5 mg/kg, 10 mg/kg 10.5 mg/kg, 11 mg/kg, 11.5 mg/kg, 12 mg/kg, 12.5 mg/kg, 13 mg/kg, 13.5 mg/kg, 14 mg/kg, 14.5 mg/kg, 15 mg/kg, 16 mg/kg, 17 mg/kg, 18 mg/kg, 19 mg/kg, 20 mg/kg, 21 mg/kg, 22 mg/kg, 23 mg/kg, 24 mg/kg, 25 mg/kg, 30 mg/kg, 35 mg/kg, 40 mg/kg, 45 mg/kg, 50 mg/kg, 55 mg/kg, 60 mg/kg, 65 mg/kg, 70 mg/kg, 75 mg/kg, 80 mg/kg, 85 mg/kg, 90 mg/kg, 95 mg/kg, 100 mg/kg, 105 mg/kg, 110 mg/kg, 115 mg/kg, 120 mg/kg, 125 mg/kg, 130 mg/kg, 135 mg/kg, 140 mg/kg, 145 mg/kg, 150 mg/kg, 155 mg/kg, 160 mg/kg, 165 mg/kg, 170 mg/kg, 175 mg/kg, 180 mg/kg, 185 mg/kg, 190 mg/kg, 195 mg/kg, 200 mg/kg, and any fraction or integer in between any two of the preceding amounts.

In some embodiments, an nNOS inhibitor composition comprising one or more miRNAs, may be administered as a suitable dosage form, as described herein, to treat, ameliorate, and/or reduce the severity of NO-lung cancer or one or more symptoms thereof in an NO-lung cancer subject. In certain embodiments, an nNOS inhibitor composition comprising one or more miRNAs selected from the group consisting of miR-146a, hsa-miR-146a-5p, hsa-miR-1273f, hsa-miR-143-3p, hsa-miR-147a, hsa-miR-3622a-5p, hsa-miR-3911, hsa-miR-4269, hsa-miR-4708-5p, hsa-miR-4710, hsa-miR-4770, hsa-miR-4792, hsa-miR-574-5p, hsa-miR-6088, hsa-miR-644a, hsa-miR-6715b-5p, hsa-miR-6768-5p, hsa-miR-6867-5p, hsa-miR-7855-5p, hsa-miR-8485, and any combination thereof, may be administered as a suitable dosage form, as described herein, to treat, ameliorate, and/or reduce the severity of NO-lung cancer or one or more symptoms thereof in an NO-lung cancer subject. In some embodiments, an nNOS inhibitor composition comprising one or more miRNAs selected from the group consisting of miR-146a, hsa-miR-146a-5p, hsa-miR-1273f, hsa-miR-143-3p, hsa-miR-147a, hsa-miR-3622a-5p, hsa-miR-3911, hsa-miR-4269, hsa-miR-4708-5p, hsa-miR-4710, hsa-miR-4770, hsa-miR-4792, hsa-miR-574-5p, hsa-miR-6088, hsa-miR-644a, hsa-miR-6715b-5p, hsa-miR-6768-5p, hsa-miR-6867-5p, hsa-miR-7855-5p, hsa-miR-8485, and any combination thereof, may be administered to an NO-lung cancer subject to treat, ameliorate, and/or reduce the severity of NO-lung cancer or one or more symptoms thereof in the NO-lung cancer subject: (i) via a route selected from the group consisting of buccally, inhalation, intraarterially, intraarticularly, intracardially, intracerebroventricularly, intradermally, intramedullary, intramuscularly, intraperitoneally, intrathecally, intravenously, intraventricularly, nasally, ophthalmically, orally, rectally, sublabially, sublingually, subcutaneously, topically, and transdermally; and/or (ii) with a frequency selected from the group consisting of four times daily, three times daily, twice daily, once daily, once every two days, once every three days, once every four days, once every five days, once every six days, once a week, once every eight days, once every nine days, once every ten days, once every two weeks, once every three weeks, and once a month; and/or (iii) at a dosage (based on subject weight) selected from the group consisting of 0.1 μg/kg, 0.5 μg/kg, 1 μg/kg, 1.5 μg/kg, 2.0 μg/kg, 2.5 μg/kg, 3.0 μg/kg, 3.5 μg/kg, 4.0 μg/kg, 4.5 μg/kg, 5.0 μg/kg, 10 μg/kg, 15 μg/kg, 20 μg/kg, 25 μg/kg, 30 μg/kg, 35 μg/kg, 40 μg/kg, 45 μg/kg, 50 μg/kg, 55 μg/kg, 60 μg/kg, 65 μg/kg, 70 μg/kg, 75 μg/kg, 80 μg/kg, 85 μg/kg, 90 μg/kg, 95 μg/kg, 100 μg/kg, 150 μg/kg, 200 μg/kg, 250 μg/kg, 300 μg/kg, 350 μg/kg, 400 μg/kg, 450 μg/kg, 500 μg/kg, 550 μg/kg, 600 μg/kg, 650 μg/kg, 700 μg/kg, 750 μg/kg, 800 μg/kg, 850 μg/kg, 900 μg/kg, 1 mg/kg, 1.5 mg/kg, 2.0 mg/kg, 2.5 mg/kg, 3 mg/kg, 3.5 mg/kg, 4.0 mg/kg, 4.5 mg/kg, 5 mg/kg, 5.5 mg/kg, 6 mg/kg, 6.5 mg/kg, 7 mg/kg, 7.5 mg/kg, 8 mg/kg, 8.5 mg/kg, 9 mg/kg, 9.5 mg/kg, 10 mg/kg 10.5 mg/kg, 11 mg/kg, 11.5 mg/kg, 12 mg/kg, 12.5 mg/kg, 13 mg/kg, 13.5 mg/kg, 14 mg/kg, 14.5 mg/kg, 15 mg/kg, 16 mg/kg, 17 mg/kg, 18 mg/kg, 19 mg/kg, 20 mg/kg, 21 mg/kg, 22 mg/kg, 23 mg/kg, 24 mg/kg, 25 mg/kg, 30 mg/kg, 35 mg/kg, 40 mg/kg, 45 mg/kg, 50 mg/kg, 55 mg/kg, 60 mg/kg, 65 mg/kg, 70 mg/kg, 75 mg/kg, 80 mg/kg, 85 mg/kg, 90 mg/kg, 95 mg/kg, 100 mg/kg, 105 mg/kg, 110 mg/kg, 115 mg/kg, 120 mg/kg, 125 mg/kg, 130 mg/kg, 135 mg/kg, 140 mg/kg, 145 mg/kg, 150 mg/kg, 155 mg/kg, 160 mg/kg, 165 mg/kg, 170 mg/kg, 175 mg/kg, 180 mg/kg, 185 mg/kg, 190 mg/kg, 195 mg/kg, 200 mg/kg, and any fraction or integer in between any two of the preceding amounts.

In some embodiments, an nNOS inhibitor composition comprising one or more CRISPR/Cas systems, may be administered as a suitable dosage form, as described herein, to treat, ameliorate, and/or reduce the severity of NO-lung cancer or one or more symptoms thereof in an NO-lung cancer subject. In certain embodiments, an nNOS inhibitor composition comprising one or more CRISPR/Cas systems comprising an endonuclease selected from the group consisting of Cas1, Cas2, Cas3, Cas4, Cas5, Cas6, Cas7, Cas8a, Cas8b, Cas8c, Cas9, Cas10, Cas10d, Cas12, Cas12a, Cas12b, Cas12c, Cas12d, Cas12e, Cas12f, Cas12g, Cas12h, Cas12i, Cas12k, C2c4, C2c8, C2c9, Cas13, Cas13a, Cas13b, Cas13c, Cas13d, Cas13x.1, Cse1, Cse2, Csy1, Csy2, Csy3, GSU0054, Csm2, Cmr5, Csx10, Csx11, Csf1, and Csn2, may be administered as a suitable dosage form, as described herein, to treat, ameliorate, and/or reduce the severity of NO-lung cancer or one or more symptoms thereof in an NO-lung cancer subject. In some embodiments, an nNOS inhibitor composition comprising one or more CRISPR/Cas systems comprising an endonuclease selected from the group consisting of Cas1, Cas2, Cas3, Cas4, Cas5, Cas6, Cas7, Cas8a, Cas8b, Cas8c, Cas9, Cas10, Cas10d, Cas12, Cas12a, Cas12b, Cas12c, Cas12d, Cas12e, Cas12f, Cas12g, Cas12h, Cas12i, Cas12k, C2c4, C2c8, C2c9, Cas13, Cas13a, Cas13b, Cas13c, Cas13d, Cas13x.1, Cse1, Cse2, Csy1, Csy2, Csy3, GSU0054, Csm2, Cmr5, Csx10, Csx11, Csf1, and Csn2, may be administered to an NO-lung cancer subject to treat, ameliorate, and/or reduce the severity of NO-lung cancer or one or more symptoms thereof in the NO-lung cancer subject: (i) via a route selected from the group consisting of buccally, inhalation, intraarterially, intraarticularly, intracardially, intracerebroventricularly, intradermally, intramedullary, intramuscularly, intraperitoneally, intrathecally, intravenously, intraventricularly, nasally, ophthalmically, orally, rectally, sublabially, sublingually, subcutaneously, topically, and transdermally; and/or (ii) with a frequency selected from the group consisting of four times daily, three times daily, twice daily, once daily, once every two days, once every three days, once every four days, once every five days, once every six days, once a week, once every eight days, once every nine days, once every ten days, once every two weeks, once every three weeks, and once a month; and/or (iii) at a dosage (based on subject weight) selected from the group consisting of 0.1 μg/kg, 0.5 μg/kg, 1 μg/kg, 1.5 μg/kg, 2.0 μg/kg, 2.5 μg/kg, 3.0 μg/kg, 3.5 μg/kg, 4.0 μg/kg, 4.5 μg/kg, 5.0 μg/kg, 10 μg/kg, 15 μg/kg, 20 μg/kg, 25 μg/kg, 30 μg/kg, 35 μg/kg, 40 μg/kg, 45 μg/kg, 50 μg/kg, 55 μg/kg, 60 μg/kg, 65 μg/kg, 70 μg/kg, 75 μg/kg, 80 μg/kg, 85 μg/kg, 90 μg/kg, 95 μg/kg, 100 μg/kg, 150 μg/kg, 200 μg/kg, 250 μg/kg, 300 μg/kg, 350 μg/kg, 400 μg/kg, 450 μg/kg, 500 μg/kg, 550 μg/kg, 600 μg/kg, 650 μg/kg, 700 μg/kg, 750 μg/kg, 800 μg/kg, 850 μg/kg, 900 μg/kg, 1 mg/kg, 1.5 mg/kg, 2.0 mg/kg, 2.5 mg/kg, 3 mg/kg, 3.5 mg/kg, 4.0 mg/kg, 4.5 mg/kg, 5 mg/kg, 5.5 mg/kg, 6 mg/kg, 6.5 mg/kg, 7 mg/kg, 7.5 mg/kg, 8 mg/kg, 8.5 mg/kg, 9 mg/kg, 9.5 mg/kg, 10 mg/kg 10.5 mg/kg, 11 mg/kg, 11.5 mg/kg, 12 mg/kg, 12.5 mg/kg, 13 mg/kg, 13.5 mg/kg, 14 mg/kg, 14.5 mg/kg, 15 mg/kg, 16 mg/kg, 17 mg/kg, 18 mg/kg, 19 mg/kg, 20 mg/kg, 21 mg/kg, 22 mg/kg, 23 mg/kg, 24 mg/kg, 25 mg/kg, 30 mg/kg, 35 mg/kg, 40 mg/kg, 45 mg/kg, 50 mg/kg, 55 mg/kg, 60 mg/kg, 65 mg/kg, 70 mg/kg, 75 mg/kg, 80 mg/kg, 85 mg/kg, 90 mg/kg, 95 mg/kg, 100 mg/kg, 105 mg/kg, 110 mg/kg, 115 mg/kg, 120 mg/kg, 125 mg/kg, 130 mg/kg, 135 mg/kg, 140 mg/kg, 145 mg/kg, 150 mg/kg, 155 mg/kg, 160 mg/kg, 165 mg/kg, 170 mg/kg, 175 mg/kg, 180 mg/kg, 185 mg/kg, 190 mg/kg, 195 mg/kg, 200 mg/kg, and any fraction or integer in between any two of the preceding amounts.

In some embodiments, an nNOS inhibitor composition comprising one or more antibodies, may be administered as a suitable dosage form, as described herein, to treat, ameliorate, and/or reduce the severity of NO-lung cancer or one or more symptoms thereof in an NO-lung cancer subject. In certain embodiments, an nNOS inhibitor composition comprising one or more antibodies selected from the group consisting of 37-2800 (from ThermoFisher), EP1855Y (from abeam), EPR 24351-6 (from abeam), and any combination thereof, may be administered as a suitable dosage form, as described herein, to treat, ameliorate, and/or reduce the severity of NO-lung cancer or one or more symptoms thereof in an NO-lung cancer subject. In some embodiments, an nNOS inhibitor composition comprising one or more antibodies selected from the group consisting of 37-2800 (from ThermoFisher), EP1855Y (from abeam), EPR 24351-6 (from abeam), and any combination thereof, may be administered to an NO-lung cancer subject to treat, ameliorate, and/or reduce the severity of NO-lung cancer or one or more symptoms thereof in the NO-lung cancer subject: (i) via a route selected from the group consisting of buccally, inhalation, intraarterially, intraarticularly, intracardially, intracerebroventricularly, intradermally, intramedullary, intramuscularly, intraperitoneally, intrathecally, intravenously, intraventricularly, nasally, ophthalmically, orally, rectally, sublabially, sublingually, subcutaneously, topically, and transdermally; and/or (ii) with a frequency selected from the group consisting of four times daily, three times daily, twice daily, once daily, once every two days, once every three days, once every four days, once every five days, once every six days, once a week, once every eight days, once every nine days, once every ten days, once every two weeks, once every three weeks, and once a month; and/or (iii) at a dosage (based on subject weight) selected from the group consisting of 0.1 μg/kg, 0.5 μg/kg, 1 μg/kg, 1.5 μg/kg, 2.0 μg/kg, 2.5 μg/kg, 3.0 μg/kg, 3.5 μg/kg, 4.0 μg/kg, 4.5 μg/kg, 5.0 μg/kg, 10 μg/kg, 15 μg/kg, 20 μg/kg, 25 μg/kg, 30 μg/kg, 35 μg/kg, 40 μg/kg, 45 μg/kg, 50 μg/kg, 55 μg/kg, 60 μg/kg, 65 μg/kg, 70 μg/kg, 75 μg/kg, 80 μg/kg, 85 μg/kg, 90 μg/kg, 95 μg/kg, 100 μg/kg, 150 μg/kg, 200 μg/kg, 250 μg/kg, 300 μg/kg, 350 μg/kg, 400 μg/kg, 450 μg/kg, 500 μg/kg, 550 μg/kg, 600 μg/kg, 650 μg/kg, 700 μg/kg, 750 μg/kg, 800 μg/kg, 850 μg/kg, 900 μg/kg, 1 mg/kg, 1.5 mg/kg, 2.0 mg/kg, 2.5 mg/kg, 3 mg/kg, 3.5 mg/kg, 4.0 mg/kg, 4.5 mg/kg, 5 mg/kg, 5.5 mg/kg, 6 mg/kg, 6.5 mg/kg, 7 mg/kg, 7.5 mg/kg, 8 mg/kg, 8.5 mg/kg, 9 mg/kg, 9.5 mg/kg, 10 mg/kg 10.5 mg/kg, 11 mg/kg, 11.5 mg/kg, 12 mg/kg, 12.5 mg/kg, 13 mg/kg, 13.5 mg/kg, 14 mg/kg, 14.5 mg/kg, 15 mg/kg, 16 mg/kg, 17 mg/kg, 18 mg/kg, 19 mg/kg, 20 mg/kg, 21 mg/kg, 22 mg/kg, 23 mg/kg, 24 mg/kg, 25 mg/kg, 30 mg/kg, 35 mg/kg, 40 mg/kg, 45 mg/kg, 50 mg/kg, 55 mg/kg, 60 mg/kg, 65 mg/kg, 70 mg/kg, 75 mg/kg, 80 mg/kg, 85 mg/kg, 90 mg/kg, 95 mg/kg, 100 mg/kg, 105 mg/kg, 110 mg/kg, 115 mg/kg, 120 mg/kg, 125 mg/kg, 130 mg/kg, 135 mg/kg, 140 mg/kg, 145 mg/kg, 150 mg/kg, 155 mg/kg, 160 mg/kg, 165 mg/kg, 170 mg/kg, 175 mg/kg, 180 mg/kg, 185 mg/kg, 190 mg/kg, 195 mg/kg, 200 mg/kg, and any fraction or integer in between any two of the preceding amounts.

In some embodiments, an nNOS inhibitor composition comprising one or more amino acid-based compounds, may be administered as a suitable dosage form, as described herein, to treat, ameliorate, and/or reduce the severity of NO-lung cancer or one or more symptoms thereof in an NO-lung cancer subject. In certain embodiments, an nNOS inhibitor composition comprising one or more amino acid-compounds that bind an active site of nNOS selected from the group consisting of Phenylalanine 589, Tryptophan 592, Tyrosine 593, Glutamate 597, Aspartate 602, Arginine 601, Methionine 341, Histidine 342, and Tyrosine711, may be administered as a suitable dosage form, as described herein, to treat, ameliorate, and/or reduce the severity of NO-lung cancer or one or more symptoms thereof in an NO-lung cancer subject. In some embodiments, an nNOS inhibitor composition comprising one or more amino acid-compounds that bind an active site of nNOS selected from the group consisting of Phenylalanine 589, Tryptophan 592, Tyrosine 593, Glutamate 597, Aspartate 602, Arginine 601, Methionine 341, Histidine 342, and Tyrosine711, may be administered to an NO-lung cancer subject to treat, ameliorate, and/or reduce the severity of NO-lung cancer or one or more symptoms thereof in the NO-lung cancer subject: (i) via a route selected from the group consisting of buccally, inhalation, intraarterially, intraarticularly, intracardially, intracerebroventricularly, intradermally, intramedullary, intramuscularly, intraperitoneally, intrathecally, intravenously, intraventricularly, nasally, ophthalmically, orally, rectally, sublabially, sublingually, subcutaneously, topically, and transdermally; and/or (ii) with a frequency selected from the group consisting of four times daily, three times daily, twice daily, once daily, once every two days, once every three days, once every four days, once every five days, once every six days, once a week, once every eight days, once every nine days, once every ten days, once every two weeks, once every three weeks, and once a month; and/or (iii) at a dosage (based on subject weight) selected from the group consisting of 0.1 μg/kg, 0.5 μg/kg, 1 μg/kg, 1.5 μg/kg, 2.0 μg/kg, 2.5 μg/kg, 3.0 μg/kg, 3.5 μg/kg, 4.0 μg/kg, 4.5 μg/kg, 5.0 μg/kg, 10 μg/kg, 15 μg/kg, 20 μg/kg, 25 μg/kg, 30 μg/kg, 35 μg/kg, 40 μg/kg, 45 μg/kg, 50 μg/kg, 55 μg/kg, 60 μg/kg, 65 μg/kg, 70 μg/kg, 75 μg/kg, 80 μg/kg, 85 μg/kg, 90 μg/kg, 95 μg/kg, 100 μg/kg, 150 μg/kg, 200 μg/kg, 250 μg/kg, 300 μg/kg, 350 μg/kg, 400 μg/kg, 450 μg/kg, 500 μg/kg, 550 μg/kg, 600 μg/kg, 650 μg/kg, 700 μg/kg, 750 μg/kg, 800 μg/kg, 850 μg/kg, 900 μg/kg, 1 mg/kg, 1.5 mg/kg, 2.0 mg/kg, 2.5 mg/kg, 3 mg/kg, 3.5 mg/kg, 4.0 mg/kg, 4.5 mg/kg, 5 mg/kg, 5.5 mg/kg, 6 mg/kg, 6.5 mg/kg, 7 mg/kg, 7.5 mg/kg, 8 mg/kg, 8.5 mg/kg, 9 mg/kg, 9.5 mg/kg, 10 mg/kg 10.5 mg/kg, 11 mg/kg, 11.5 mg/kg, 12 mg/kg, 12.5 mg/kg, 13 mg/kg, 13.5 mg/kg, 14 mg/kg, 14.5 mg/kg, 15 mg/kg, 16 mg/kg, 17 mg/kg, 18 mg/kg, 19 mg/kg, 20 mg/kg, 21 mg/kg, 22 mg/kg, 23 mg/kg, 24 mg/kg, 25 mg/kg, 30 mg/kg, 35 mg/kg, 40 mg/kg, 45 mg/kg, 50 mg/kg, 55 mg/kg, 60 mg/kg, 65 mg/kg, 70 mg/kg, 75 mg/kg, 80 mg/kg, 85 mg/kg, 90 mg/kg, 95 mg/kg, 100 mg/kg, 105 mg/kg, 110 mg/kg, 115 mg/kg, 120 mg/kg, 125 mg/kg, 130 mg/kg, 135 mg/kg, 140 mg/kg, 145 mg/kg, 150 mg/kg, 155 mg/kg, 160 mg/kg, 165 mg/kg, 170 mg/kg, 175 mg/kg, 180 mg/kg, 185 mg/kg, 190 mg/kg, 195 mg/kg, 200 mg/kg, and any fraction or integer in between any two of the preceding amounts.

In some embodiments, a NO-lung cancer subject, as described herein, may be a female child or a male child. In certain embodiments, a NO-lung cancer subject, as described herein, may be a female adult or a male adult.

In some embodiments, a NO-lung cancer a subject, as described herein, may have an abnormal expression of one or more antigens compared to an equivalent subject that does not have a NO-lung cancer. As used herein, the term “equivalent subject,” refers to a subject of the same sex (male/female) and age range (child/adult) of the NO-lung cancer subject. For purposes of disclosure, the term “antigen,” when used with respect to abnormal expression, may define one or more cluster of differentiation (“CD”) antigens, such as, for example, CD15, CD44, CD70, CD133, as well as any CD antigen that has an abnormal expression in a NO-lung cancer that would be readily envisaged by one skilled in the art. In certain embodiments, abnormal expression of an antigen may define an abnormal expression of CD15, an abnormal expression of CD44, an abnormal expression of CD70, an abnormal expression of CD133, an abnormal expression of a CD antigen that has an abnormal expression in a NO-lung cancer that would be readily envisaged by one skilled in the art, and any combination thereof. In some embodiments, an “abnormal expression,” can refer to a reduced expression of an antigen, as described herein. In certain embodiments, an “abnormal expression,” can refer to an overexpression of an antigen, as described herein. Whether “abnormal expression,” refers to a reduced expression of an antigen or an overexpression of an antigen would be immediately envisaged by the skilled artisan when viewing the term in the context of the particular antigen and NO-lung cancer disclosed. For example, in the context of CD133 and small cell carcinomas (NO-lung cancers), one skilled in the art would immediately envisage that “abnormal expression” refers to an overexpression of CD133. Treatment, amelioration, and/or reduction in the severity of a NO-lung cancer or one or more symptoms thereof may be characterized by a restoration to a normal level of expression of one or more antigens from their abnormal state, as described herein. For example, in the context of CD133 and small cell carcinomas, treatment, amelioration, and/or reduction in the severity of a NO-lung cancer or one or more symptoms thereof may be characterized by a reduced expression of CD133.

In certain embodiments, a NO-lung cancer subject, as described herein, may have an abnormal expression of one or more proteins compared to an equivalent subject that does not have a NO-lung cancer. For purposes of disclosure, the term “protein,” when used with respect to abnormal expression, may define one or more proteins and/or enzymes, such as, for example, ALDH1A3, mTOR, p-mTOR, TSC-2, AKT, pAKT, RPS6, p-RPS6, Syp, VEGF, PIGF, as well as any protein or enzyme that has an abnormal expression in a NO-lung cancer that would be readily envisaged by one skilled in the art. In certain embodiments, abnormal expression of a protein may define an abnormal expression of ALDH1A3, an abnormal expression of mTOR, an abnormal expression of p-mTOR, an abnormal expression of TSC-2, an abnormal expression of AKT, an abnormal expression of pAKT, an abnormal expression of RPS6, an abnormal expression of p-RPS6, an abnormal expression of Syp, an abnormal expression of VEGF, an abnormal expression of PIGF, an abnormal expression of a protein or enzyme that has an abnormal expression in a NO-lung cancer that would be readily envisaged by one skilled in the art, and any combination thereof. In some embodiments, an “abnormal expression,” can refer to a reduced expression of a protein, as described herein. In certain embodiments, an “abnormal expression,” can refer to an overexpression of a protein, as described herein. Whether “abnormal expression,” refers to a reduced expression of a protein or an overexpression of a protein would be immediately envisaged by the skilled artisan when viewing the term in the context of the particular protein and NO-lung cancer disclosed. For example, in the context of non-small cell lung carcinoma (a NO-lung cancer), one skilled in the art would immediately envisage that “abnormal expression” refers to an overexpression of mTOR. Treatment, amelioration, and/or reduction in the severity of a NO-lung cancer or one or more symptoms thereof may be characterized by a restoration to a normal level of expression of one or more proteins from their abnormal state, as described herein. For example, in the context of non-small cell lung carcinoma, treatment, amelioration, and/or reduction in the severity of a NO-lung cancer or one or more symptoms thereof may be characterized by a reduced expression of mTOR.

In certain embodiments, a NO-lung cancer subject, as described herein, may have an increased expression of one or more NOS isoforms compared to an equivalent subject that does not have a NO-lung cancer. For purposes of disclosure, the term “NOS isoform,” refers to nNOS, iNOS, and/or eNOS. In certain embodiments, a NO-lung cancer subject, as described herein, may have an increased expression of nNOS compared to an equivalent subject that does not have a NO-lung cancer. In some embodiments, a NO-lung cancer subject, as described herein, may have an increased expression of iNOS compared to an equivalent subject that does not have a NO-lung cancer. In certain embodiments, a NO-lung cancer subject, as described herein, may have an increased expression of eNOS compared to an equivalent subject that does not have a NO-lung cancer. In some embodiments, a NO-lung cancer subject, as described herein, may have an increased expression of nNOS and iNOS compared to an equivalent subject that does not have a NO-lung cancer. In certain embodiments, a NO-lung cancer subject, as described herein, may have an increased expression of nNOS and eNOS compared to an equivalent subject that does not have a NO-lung cancer. In some embodiments, a NO-lung cancer subject, as described herein, may have an increased expression of iNOS and eNOS compared to an equivalent subject that does not have a NO-lung cancer. In certain embodiments, a NO-lung cancer subject, as described herein, may have an increased expression of nNOS, iNOS, and eNOS compared to an equivalent subject that does not have a NO-lung cancer. Whether a “NOS isoform,” has an increased expression would be immediately envisaged by the skilled artisan when viewing the term in the context of the particular NOS isoform and NO-lung cancer disclosed. For example, in the context of non-small cell lung carcinoma (a NO-lung cancer), one skilled in the art would immediately envisage that iNOS has an increased expression. Treatment, amelioration, and/or reduction in the severity of a NO-lung cancer or one or more symptoms thereof may be characterized by a reduced expression of one or more NOS isoforms from their increased state, as described herein. For example, in the context of non-small cell lung carcinoma, treatment, amelioration, and/or reduction in the severity of a NO-lung cancer or one or more symptoms thereof may be characterized by a reduced expression of iNOS. For purposes of disclosure, reduction/inhibition of the expression of one or more NOS isoforms may be characterized by a reduction in expression of one or more NOS isoforms by about at least 10%, about at least 20%, about at least 30%, or about at least 40%. In some embodiments, reduction/inhibition of the expression of one or more NOS isoforms may further be characterized by a lack reduction of other, non-implicated NOS isoforms, such as, for example, no more than about 10%, no more than about 5%, no more than about 2%, or no more than about 1% reduction/inhibition of the expression of non-implicated NOS isoforms. As used herein, the term “non-implicated NOS isoforms,” refers to NOS isoforms that do not have an increased expression in a particular NO-lung cancer. By way of example, in the context of non-small cell lung carcinoma, if iNOS has an increased expression, nNOS and/or eNOS would define a “non-implicated” NOS isoform. Whether a NOS isoform is a “non-implicated NOS isoform,” would be immediately envisaged by the skilled artisan when viewing the term in the context of the particular NOS isoform disclosed, as well as the NO-lung cancer.

In some embodiments, a NO-lung cancer subject, as described herein, may have an elevated level of one or more NO-indicators compared to an equivalent subject that does not have a NO-lung cancer. As used herein, the term “NO-indicator,” can refer to 3-nitrotyrosine (3-Ntyr), NADPH-diaphorase, total nitrite concentration, total nitrate concentration, total nitrite and nitrate concentration, as well as any NO-indicator that has an elevated level in a NO-lung cancer that would be readily envisaged by one skilled in the art. For example, in the context of small cell carcinoma (a NO-lung cancer), one skilled in the art would immediately envisage that 3-Ntyr can be present at an elevated level. Treatment, amelioration, and/or reduction in the severity of a NO-lung cancer or one or more symptoms thereof may be characterized by a reduction in the level of one or more NO-indicators. For example, in the context of small cell carcinoma, treatment, amelioration, and/or reduction in the severity of a NO-lung cancer or one or more symptoms thereof may be characterized by a reduction in the level of 3-Ntyr.

In certain embodiments, a NOS inhibitor composition, as described herein, may be administered as a suitable dosage form, as described herein, to treat, ameliorate, and/or reduce the severity of a NO-lung cancer or one or more symptoms thereof, as an alternative to a conventional therapy for treating, ameliorating, and/or reducing the severity of a NO-lung cancer or one or more symptoms thereof. For purposes of disclosure, a “conventional therapy,” as described herein, may refer to a treatment that would be recommended by one skilled in the art to treat a NO-lung cancer, and may define one or more of radiation, chemotherapy, immunotherapy, cell-based therapy, gene therapy, and the like—one skilled in the art would immediately envisage a “conventional therapy” based on the particular NO-lung cancer disclosed, as well as characteristics of a NO-lung cancer subject, such as, for example, the subject's sex, age, stage of the NO-lung cancer, genotype of the NO-lung cancer, and the like. Treatment, amelioration, and/or reduction in the severity of a NO-lung cancer or one or more symptoms thereof may be characterized by an improved outcome compared to a conventional therapy, as described herein. For purposes of the disclosure, an “improved outcome” may define any one of the following: (i) a larger reduction in tumor volume and/or mass compared to a subject administered an equivalent dose of a conventional therapy; (ii) an improvement in the expected rate of survival of a subject, at, for example, 1 month, 3 months, 6 months, 9 months, 1 year, 2 years, 3 years, 5 years, 10 years, or more, compared to the expected rate of survival of a subject administered an equivalent dose of a conventional therapy; (iii) a reduction in the prevalence of side effects resulting from the NOS inhibitor composition compared to a subject administered an equivalent dose of a conventional therapy; (iv) a reduction in the severity of side effects resulting from the NOS inhibitor composition compared to a subject administered an equivalent dose of a conventional therapy; (v) a reduction in the dosage required to achieve an equivalent reduction in tumor volume compared to a subject administered a conventional therapy; (vi) a reduction in the dosage required to achieve an equivalent expected rate of survival of a subject compared to a subject administered a conventional therapy; (vii) a reduction in the likelihood of a recurrence of the NO-lung cancer occurring in a subject compared to a subject administered an equivalent dose of a conventional therapy; (viii) a reduction in the likelihood of a metastasis occurring in a subject compared to a subject administered an equivalent dose of a conventional therapy; (ix) a reduction in the time required to be at a medical facility for treatment compared to a subject administered an equivalent dose of a conventional therapy; (x) a reduction in the cost associated with treatment compared to a subject administered an equivalent dose of a conventional therapy; as well as any other improved outcome not expressly recited that would be readily envisaged by one skilled in the art.

In some embodiments, a NOS inhibitor composition, as described herein, may be administered as a suitable dosage form, as described herein, to treat, ameliorate, and/or reduce the severity of a NO-lung cancer or one or more symptoms thereof, wherein the NOS inhibitor composition comprises a first chemical NOS inhibitor and a second chemical NOS inhibitor. In certain embodiments, the first chemical NOS inhibitor may be administered concurrently with the second chemical NOS inhibitor. In some embodiments, the first chemical NOS inhibitor may be administered prior to the second chemical NOS inhibitor. In certain embodiments, the first chemical NOS inhibitor may be administered subsequently to the second chemical NOS inhibitor. Treatment, amelioration, and/or reduction in the severity of a NO-lung cancer or one or more symptoms thereof may be characterized by an improved outcome compared an alternative treatment. As used herein, the term “alternative treatment,” may define one or more of a conventional therapy, as described herein, a NOS inhibitor composition comprising a single chemical NOS inhibitor, as described herein, and a NOS inhibitor composition comprising a single biological NOS inhibitor, as described herein. For purposes of the disclosure, an “improved outcome” may define any one of the following: (i) a larger reduction in tumor volume and/or mass compared an alternative treatment; (ii) an improvement in the expected rate of survival of a subject, at, for example, 1 month, 3 months, 6 months, 9 months, 1 year, 2 years, 3 years, 5 years, 10 years, or more, compared to the expected rate of survival of a subject administered an alternative treatment; (iii) a reduction in the prevalence of side effects resulting from the NOS inhibitor composition comprising at least two chemical NOS inhibitors compared to a subject administered an alternative treatment; (iv) a reduction in the severity of side effects resulting from the NOS inhibitor composition comprising at least two chemical NOS inhibitors compared to a subject administered an alternative treatment; (v) a reduction in the dosage required to achieve an equivalent reduction in tumor volume compared to a subject administered an alternative treatment; (vi) a reduction in the dosage required to achieve an equivalent expected rate of survival of a subject compared to a subject administered an alternative treatment; (vii) a reduction in the likelihood of a recurrence of the NO-lung cancer in a subject compared to a subject administered an alternative treatment; (viii) a reduction in the likelihood of a metastasis occurring in a subject compared to a subject administered an alternative treatment; (ix) a reduction in the time required to be at a medical facility for treatment compared to a subject administered an alternative treatment; (x) a reduction in the cost associated with treatment compared to a subject administered an alternative treatment; as well as any other improved outcome not expressly recited that would be readily envisaged by one skilled in the art.

In certain embodiments, a NOS inhibitor composition, as described herein, may be used to treat, ameliorate, and/or reduce the severity of a NO-lung cancer or one or more symptoms thereof, wherein the NOS inhibitor composition comprises at least one chemical NOS inhibitor and at least one biological NOS inhibitor. In some embodiments, the at least one chemical NOS inhibitor may be administered concurrently with the at least one biological NOS inhibitor. In certain embodiments, the at least one chemical NOS inhibitor may be administered prior to the at least one biological NOS inhibitor. In some embodiments, the at least one chemical NOS inhibitor may be administered subsequently to the at least one biological NOS inhibitor. Treatment, amelioration, and/or reduction in the severity of a NO-lung cancer or one or more symptoms thereof may be characterized by an improved outcome compared an alternative treatment. As used herein, the term “alternative treatment,” may define one or more of a conventional therapy, as described herein, a NOS inhibitor composition comprising a single chemical NOS inhibitor, as described herein, and a NOS inhibitor composition comprising a single biological NOS inhibitor, as described herein. For purposes of the disclosure, an “improved outcome” may define any one of the following: (i) a larger reduction in tumor volume and/or mass compared an alternative treatment; (ii) an improvement in the expected rate of survival of a subject, at, for example, 1 month, 3 months, 6 months, 9 months, 1 year, 2 years, 3 years, 5 years, 10 years, or more, compared to the expected rate of survival of a subject administered an alternative treatment; (iii) a reduction in the prevalence of side effects resulting from the NOS inhibitor composition comprising at least one chemical NOS inhibitor and at least one biological NOS inhibitor compared to a subject administered an alternative treatment; (iv) a reduction in the severity of side effects resulting from the NOS inhibitor composition comprising at least one chemical NOS inhibitor and at least one biological NOS inhibitor compared to a subject administered an alternative treatment; (v) a reduction in the dosage required to achieve an equivalent reduction in tumor volume compared to a subject administered an alternative treatment; (vi) a reduction in the dosage required to achieve an equivalent expected rate of survival of a subject compared to a subject administered an alternative treatment; (vii) a reduction in the likelihood of a recurrence of the NO-lung cancer in a subject compared to a subject administered an alternative treatment; (viii) a reduction in the likelihood of a metastasis occurring in a subject compared to a subject administered an alternative treatment; (ix) a reduction in the time required to be at a medical facility for treatment compared to a subject administered an alternative treatment; (x) a reduction in the cost associated with treatment compared to a subject administered an alternative treatment; as well as any other improved outcome not expressly recited that would be readily envisaged by one skilled in the art.

In some embodiments, a NOS inhibitor composition, as described herein, may be combined with a conventional therapy, as described herein, to treat, ameliorate, and/or reduce the severity of a NO-lung cancer or one or more symptoms thereof. In certain embodiments, the NOS inhibitor composition may be administered concurrently with the conventional therapy. In some embodiments, the NOS inhibitor composition may be administered prior to the conventional therapy. In certain embodiments, the NOS inhibitor composition may be administered subsequently to the conventional therapy. Treatment, amelioration, and/or reduction in the severity of a NO-lung cancer or one or more symptoms thereof may be characterized by an improved outcome compared an alternative treatment. As used herein, the term “alternative treatment,” may define one or more of a conventional therapy, as described herein, a NOS inhibitor composition comprising a single chemical NOS inhibitor, as described herein, and a NOS inhibitor composition comprising a single biological NOS inhibitor, as described herein. For purposes of the disclosure, an “improved outcome” may define any one of the following: (i) a larger reduction in tumor volume and/or mass compared an alternative treatment; (ii) an improvement in the expected rate of survival of a subject, at, for example, 1 month, 3 months, 6 months, 9 months, 1 year, 2 years, 3 years, 5 years, 10 years, or more, compared to the expected rate of survival of a subject administered an alternative treatment; (iii) a reduction in the prevalence of side effects resulting from the NOS inhibitor composition and conventional therapy compared to a subject administered an alternative treatment; (iv) a reduction in the severity of side effects resulting from the NOS inhibitor composition and conventional therapy compared to a subject administered an alternative treatment; (v) a reduction in the dosage required to achieve an equivalent reduction in tumor volume compared to a subject administered an alternative treatment; (vi) a reduction in the dosage required to achieve an equivalent expected rate of survival of a subject compared to a subject administered an alternative treatment; (vii) a reduction in the likelihood of a recurrence of the NO-lung cancer a in a subject compared to a subject administered an alternative treatment; (viii) a reduction in the likelihood of a metastasis occurring in a subject compared to a subject administered an alternative treatment; (ix) a reduction in the time required to be at a medical facility for treatment compared to a subject administered an alternative treatment; (x) a reduction in the cost associated with treatment compared to a subject administered an alternative treatment; as well as any other improved outcome not expressly recited that would be readily envisaged by one skilled in the art.

In some embodiments, a “conventional therapy,” as that term is used with respect to combined administration with a NOS inhibitor composition, may refer to a conventional chemotherapy. For purposes of the disclosure, a “conventional chemotherapy,” as described herein may be a compound selected from the group consisting of abemaciclib, abiraterone acetate, acalabrutinib, adagrasib, afatinib dimaleate, albumin-bound paclitaxel, albumin-bound rapamycin (Sirolimus Protein-Bound Particles), alectinib, alpelisib, amifostine, aminolevulinic acid hydrochloride, anastrozole, apalutamide, aprepitant, arsenic trioxide, asciminib hydrochloride, avapritinib, axitinib, azacytidine, belinostat, belzutifan, bendamustine hydrochloride, bexarotene, bicalutamide, binimetinib, bleomycin, bleomycin sulfate, bortezomib, bosutinib, brigatinib, busulfan, cabazitaxel, cabozantinib-S-malate, capecitabine, capmatinib hydrochloride, carboplatin, carfilzomib, carmustine, cedazuridine, ceritinib, chlorambucil, cisplatin, cladribine, clofarabine, cobimetinib fumarate, copanlisib hydrochloride, crizotinib, cyclophosphamide, cytarabine, dabrafenib mesylate, dacarbazine, dacomitinib, dactinomycin, darolutamide, dasatinib, daunorubicin hydrochloride, decitabine, defibrotide sodium, degarelix, dexamethasone, dexrazoxane hydrochloride, docetaxel, doxorubicin hydrochloride, duvelisib, elacestrant dihydrochloride, eltrombopag olamine, enasidenib mesylate, encorafenib, entrectinib, enzalutamide, epirubicin hydrochloride, erdafitinib, eribulin mesylate, erlotinib hydrochloride, etoposide, etoposide phosphate, everolimus, exemestane, fedratinib hydrochloride, fludarabine phosphate, fluorouracil, flutamide, folinic acid, fostamatinib disodium, fulvestrant, futibatinib, gefitinib, gemcitabine hydrochloride, gilteritinib fumarate, glasdegib maleate, goserelin acetate, granisetron, granisetron hydrochloride, hydroxyurea, ibrutinib, idarubicin hydrochloride, idelalisib, ifosfamide, imatinib mesylate, imiquimod, infigratinib phosphate, iobenguane I-131, irinotecan hydrochloride, ivosidenib, ixabepilone, ixazomib citrate, lanreotide acetate, lapatinib ditosylate, larotrectinib sulfate, lenalidomide, lenvatinib mesylate, letrozole, leucovorin calcium, leuprolide acetate, liposomal cytarabine, liposomal doxorubicin hydrochloride, liposomal irinotecan hydrochloride, lomustine, lorlatinib, lurbinectedin, Lu-177 dotatate, Lu-177 vipivotide tetraxetan, mechlorethamine hydrochloride, megestrol acetate, melphalan, melphalan hydrochloride, mercaptopurine, mesna, methotrexate, methotrexate sodium, methylnaltrexone bromide, midostaurin, mitomycin, mitoxantrone hydrochloride, mobocertinib succinate, nelarabine, neratinib maleate, netupitant hydrochloride, nilutamide, nilotinib, niraparib tosylate monohydrate, olaparib, olutasidenib, omacetaxine mepesuccinate, ondansetron hydrochloride, osimertinib mesylate, oxaliplatin, paclitaxel, pacritinib citrate, palbociclib, palonosetron hydrochloride, pamidronate disodium, pazopanib hydrochloride, pemetrexed disodium, pemigatinib, pexidartinib hydrochloride, pirtobrutinib, plerixafor, pomalidomide, ponatinib hydrochloride, pralatrexate, pralsetinib, prednisone, procarbazine hydrochloride, propranolol hydrochloride, Ra-223 dichloride, raloxifene hydrochloride, regorafenib, relugolix, ribociclib, ripretinib, rolapitant hydrochloride, romidepsin, rucaparib camsylate, ruxolitinib phosphate, selinexor, selpercatinib, selumetinib sulfate, sonidegib, sorafenib tosylate, sotorasib, sunitinib malate, talazoparib tosylate, talc, tamoxifen citrate, tazemetostat hydrobromide, temozolomide, temsirolimus, tepotinib hydrochloride, thalidomide, thioguanine, thiotepa, tipiracil hydrochloride, tivozanib hydrochloride, topotecan hydrochloride, toremifene, trabectedin, trametinib dimethyl sulfoxide, tretinoin, trifluridine hydrochloride, tucatinib, uridine triacetate, valrubicin, vandetanib, vemurafenib, venetoclax, vinblastine sulfate, vincristine sulfate, vinorelbine tartrate, vismodegib, vorinostat, zanubrutinib, zoledronic acid, and any combination thereof. One skilled in the art would immediately envisage an appropriate “conventional chemotherapy” based on the particular NO-lung cancer disclosed, as well as characteristics of a NO-lung cancer subject, such as, for example, the subject's sex, age, stage of the NO-lung cancer, genotype of the NO-lung cancer, and the like.

In certain embodiments, a “conventional therapy,” as that term is used with respect to combined administration with a NOS inhibitor composition, may refer to a conventional immunotherapy. For purposes of the disclosure, a “conventional immunotherapy,” as described herein may be an immunotherapy, cell-based therapy, and/or gene-based therapy selected from the group consisting of ado-trastuzumab emtansine, aldesleukin, alemtuzumab, amivantamab-vmjw, asparaginase Erwinia chrysanthemi, asparaginase Erwinia chrysanthemi (recombinant)-rywn, atezolizumab, avelumab, axicabtagene ciloleucel, bevacizumab, blinatumomab, brentuximab vedotin, brexucabtagene autoleucel, calaspargase pegol-mknl, camrelizumab, caplacizumab-yhdp, cemiplimab-rwlc, cetuximab, ciltacabtagene autoleucel, daratumumab, daratumumab and hyaluronidase-fihj, darbepoetin alfa, denileukin diftitox, denosumab, dinutuximab, dostarlimab-gxly, durvalumab, elotuzumab, emapalumab-lzsg, enfortumab vedotin-ejfv, epoetin alfa, fam-trastuzumab deruxtecan-nxki, filgrastim, gemtuzumab ozogamicin, glucarpidase, ibritumomab tiuxetan, idecabtagene vicleucel, inotuzumab ozogamicin, ipilimumab, isatuximab-irfc, lisocabtagene maraleucel, loncastuximab tesirine-lpyl, luspatercept-aamt, margetuximab-cmkb, mirvetuximab soravtansine-gynx, mogamulizumab-kpkc, mosunetuzumab-axgb, moxetumomab pasudotox-tdfk, nadofaragene firadenovec-vncg, naxitamab-gqgk, necitumumab, nivolumab, nivolumab and relatlimab-rmbw, obinutuzumab, ofatumumab, palifermin, panitumumab, pegaspargase, pegfilgrastim, pembrolizumab, pertuzumab, pertuzumab, trastuzumab, and hyaluronidase-zzxf, polatuzumab vedotin-piiq, ramucirumab, rasburicase, ravulizumab-cwvz, recombinant HPV bivalent vaccine, recombinant HPV nonavalent vaccine, recombinant HPV quadrivalent vaccine, recombinant interferon alfa-2b, rituximab, rituximab and hyaluronidase human, romiplostim, ropeginterferon alfa-2b-njft, sacituzumab govitecan-hziy, siltuximab, sintilimab, sipuleucel-T, tafasitamab-cxix, tagraxofusp-erzs, talimogene laherparepvec, tebentafusp-tebn, teclistamab-cqyv, tisagenlecleucel, tislelizumab, tisotumab vedotin-tftv, tocilizumab, toripalimab, trastuzumab, trastuzumab and hyaluronidase-oysk, tremelimumab-actl, ziv-aflibercept, and any combination thereof. One skilled in the art would immediately envisage an appropriate “conventional immunotherapy” based on the particular NO-lung cancer disclosed, as well as characteristics of a NO-lung cancer subject, such as, for example, the subject's sex, age, stage of the NO-lung cancer, genotype of the NO-lung cancer, and the like.

In certain embodiments, a NOS inhibitor composition, as described herein, may be administered as a suitable dosage form, as described herein, prior to, or alternatively, following a surgery to remove or resect a NO-lung cancer.

In some embodiments, a NOS inhibitor composition, as described herein, may be administered as a suitable dosage form, as described herein, to restore and/or maintain healthy and/or optimal expression levels of one or more antigens, as described herein, such as, for example, CD15, CD44, CD70, CD133, as well as any CD antigen that has an abnormal expression in a NO-lung cancer that would be readily envisaged by one skilled in the art, in a subject. In certain embodiments, the subject may have a NO-lung cancer or one or more symptoms thereof. As used herein, the terms “healthy” and “optimal” refer to expression levels of one or more antigens that would be typical of an equivalent subject that does not have a NO-lung cancer.

In certain embodiments, a NOS inhibitor composition, as described herein, may be administered as a suitable dosage form, as described herein, to restore and/or maintain healthy and/or optimal expression levels of one or more proteins, as described herein, such as, for example, ALDH1A3, mTOR, p-mTOR, TSC-2, AKT, pAKT, RPS6, p-RPS6, Syp, VEGF, PIGF, as well as any protein or enzyme that has an abnormal expression in a NO-lung cancer that would be readily envisaged by one skilled in the art, in a subject. In certain embodiments, the subject may have a NO-lung cancer or one or more symptoms thereof. As used herein, the terms “healthy” and “optimal” refer to expression levels of one or more proteins that would be typical of an equivalent subject that does not have a NO-lung cancer.

In some embodiments, a NOS inhibitor composition, as described herein, may be administered as a suitable dosage form, as described herein, to restore and/or maintain healthy and/or optimal expression levels of one or more NOS isoforms, as described herein, such as, for example, nNOS, iNOS, and eNOS, in a subject. In certain embodiments, the subject may have a NO-lung cancer or one or more symptoms thereof. As used herein, the terms “healthy” and “optimal” refer to expression levels of one or more NOS isoforms that would be typical of an equivalent subject that does not have a NO-lung cancer.

In certain embodiments, a NOS inhibitor composition, as described herein, may be administered as a suitable dosage form, as described herein, to restore and/or maintain healthy and/or optimal levels of one or more NO-indicators, as described herein, such as, for example, 3-Ntyr, NADPH-diaphorase, total nitrite concentration, total nitrate concentration, total nitrite and nitrate concentration, as well as any NO-indicator that has an elevated level in a NO-lung cancer that would be readily envisaged by one skilled in the art, in a subject. In certain embodiments, the subject may have a NO-lung cancer or one or more symptoms thereof. As used herein, the terms “healthy” and “optimal” refer to levels of one or more NO-indicators that would be typical of an equivalent subject that does not have a NO-lung cancer.

In certain embodiments, a NOS inhibitor composition, as described herein, may be administered as a suitable dosage form, as described herein, to treat, ameliorate, and/or reduce the severity of a non-small cell lung cancer (“NSC-lung cancer”) or one or more symptoms thereof in a subject (“NSC-lung cancer subject”). One skilled in the art would readily envisage the subtypes of lung cancer that are classified as non-small cell lung cancer, such as, for example, squamous cell carcinoma, adenocarcinoma, at the like.

In some embodiments, a NOS inhibitor composition comprising 7-NI and/or a salt thereof may be administered as a suitable dosage form, as described herein, to treat, ameliorate, and/or reduce the severity of NSC-lung cancer or one or more symptoms thereof in an NSC-lung cancer subject. In certain embodiments, an nNOS inhibitor composition comprising 7-NI and/or a salt thereof may be administered to an NSC-lung cancer subject to treat, ameliorate, and/or reduce the severity of NSC-lung cancer or one or more symptoms thereof in the NSC-lung cancer subject: (i) via a route selected from the group consisting of buccally, inhalation, intraarterially, intraarticularly, intracardially, intracerebroventricularly, intradermally, intramedullary, intramuscularly, intraperitoneally, intrathecally, intravenously, intraventricularly, nasally, ophthalmically, orally, rectally, sublabially, sublingually, subcutaneously, topically, and transdermally; and/or (ii) with a frequency selected from the group consisting of four times daily, three times daily, twice daily, once daily, once every two days, once every three days, once every four days, once every five days, once every six days, once a week, once every eight days, once every nine days, once every ten days, once every two weeks, once every three weeks, and once a month; and/or (iii) at a dosage (based on subject weight) selected from the group consisting of 0.1 μg/kg, 0.5 μg/kg, 1 μg/kg, 1.5 μg/kg, 2.0 μg/kg, 2.5 μg/kg, 3.0 μg/kg, 3.5 μg/kg, 4.0 μg/kg, 4.5 μg/kg, 5.0 μg/kg, 10 μg/kg, 15 μg/kg, 20 μg/kg, 25 μg/kg, 30 μg/kg, 35 μg/kg, 40 μg/kg, 45 μg/kg, 50 μg/kg, 55 μg/kg, 60 μg/kg, 65 μg/kg, 70 μg/kg, 75 μg/kg, 80 μg/kg, 85 μg/kg, 90 μg/kg, 95 μg/kg, 100 μg/kg, 150 μg/kg, 200 μg/kg, 250 μg/kg, 300 μg/kg, 350 μg/kg, 400 μg/kg, 450 μg/kg, 500 μg/kg, 550 μg/kg, 600 μg/kg, 650 μg/kg, 700 μg/kg, 750 μg/kg, 800 μg/kg, 850 μg/kg, 900 μg/kg, 1 mg/kg, 1.5 mg/kg, 2.0 mg/kg, 2.5 mg/kg, 3 mg/kg, 3.5 mg/kg, 4.0 mg/kg, 4.5 mg/kg, 5 mg/kg, 5.5 mg/kg, 6 mg/kg, 6.5 mg/kg, 7 mg/kg, 7.5 mg/kg, 8 mg/kg, 8.5 mg/kg, 9 mg/kg, 9.5 mg/kg, 10 mg/kg 10.5 mg/kg, 11 mg/kg, 11.5 mg/kg, 12 mg/kg, 12.5 mg/kg, 13 mg/kg, 13.5 mg/kg, 14 mg/kg, 14.5 mg/kg, 15 mg/kg, 16 mg/kg, 17 mg/kg, 18 mg/kg, 19 mg/kg, 20 mg/kg, 21 mg/kg, 22 mg/kg, 23 mg/kg, 24 mg/kg, 25 mg/kg, 30 mg/kg, 35 mg/kg, 40 mg/kg, 45 mg/kg, 50 mg/kg, 55 mg/kg, 60 mg/kg, 65 mg/kg, 70 mg/kg, 75 mg/kg, 80 mg/kg, 85 mg/kg, 90 mg/kg, 95 mg/kg, 100 mg/kg, 105 mg/kg, 110 mg/kg, 115 mg/kg, 120 mg/kg, 125 mg/kg, 130 mg/kg, 135 mg/kg, 140 mg/kg, 145 mg/kg, 150 mg/kg, 155 mg/kg, 160 mg/kg, 165 mg/kg, 170 mg/kg, 175 mg/kg, 180 mg/kg, 185 mg/kg, 190 mg/kg, 195 mg/kg, 200 mg/kg, and any fraction or integer in between any two of the preceding amounts.

In certain embodiments, a NOS inhibitor composition comprising 3-Br-7-NI and/or a salt thereof may be administered as a suitable dosage form, as described herein, to treat, ameliorate, and/or reduce the severity of NSC-lung cancer or one or more symptoms thereof in an NSC-lung cancer subject. In some embodiments, an nNOS inhibitor composition comprising 3-Br-7-NI and/or a salt thereof may be administered to an NSC-lung cancer subject to treat, ameliorate, and/or reduce the severity of NSC-lung cancer or one or more symptoms thereof in the NSC-lung cancer subject: (i) via a route selected from the group consisting of buccally, inhalation, intraarterially, intraarticularly, intracardially, intracerebroventricularly, intradermally, intramedullary, intramuscularly, intraperitoneally, intrathecally, intravenously, intraventricularly, nasally, ophthalmically, orally, rectally, sublabially, sublingually, subcutaneously, topically, and transdermally; and/or (ii) with a frequency selected from the group consisting of four times daily, three times daily, twice daily, once daily, once every two days, once every three days, once every four days, once every five days, once every six days, once a week, once every eight days, once every nine days, once every ten days, once every two weeks, once every three weeks, and once a month; and/or (iii) at a dosage (based on subject weight) selected from the group consisting of 0.1 μg/kg, 0.5 μg/kg, 1 μg/kg, 1.5 μg/kg, 2.0 μg/kg, 2.5 μg/kg, 3.0 μg/kg, 3.5 μg/kg, 4.0 μg/kg, 4.5 μg/kg, 5.0 μg/kg, 10 μg/kg, 15 μg/kg, 20 μg/kg, 25 μg/kg, 30 μg/kg, 35 μg/kg, 40 μg/kg, 45 μg/kg, 50 μg/kg, 55 μg/kg, 60 μg/kg, 65 μg/kg, 70 μg/kg, 75 μg/kg, 80 μg/kg, 85 μg/kg, 90 μg/kg, 95 μg/kg, 100 μg/kg, 150 μg/kg, 200 μg/kg, 250 μg/kg, 300 μg/kg, 350 μg/kg, 400 μg/kg, 450 μg/kg, 500 μg/kg, 550 μg/kg, 600 μg/kg, 650 μg/kg, 700 μg/kg, 750 μg/kg, 800 μg/kg, 850 μg/kg, 900 μg/kg, 1 mg/kg, 1.5 mg/kg, 2.0 mg/kg, 2.5 mg/kg, 3 mg/kg, 3.5 mg/kg, 4.0 mg/kg, 4.5 mg/kg, 5 mg/kg, 5.5 mg/kg, 6 mg/kg, 6.5 mg/kg, 7 mg/kg, 7.5 mg/kg, 8 mg/kg, 8.5 mg/kg, 9 mg/kg, 9.5 mg/kg, 10 mg/kg 10.5 mg/kg, 11 mg/kg, 11.5 mg/kg, 12 mg/kg, 12.5 mg/kg, 13 mg/kg, 13.5 mg/kg, 14 mg/kg, 14.5 mg/kg, 15 mg/kg, 16 mg/kg, 17 mg/kg, 18 mg/kg, 19 mg/kg, 20 mg/kg, 21 mg/kg, 22 mg/kg, 23 mg/kg, 24 mg/kg, 25 mg/kg, 30 mg/kg, 35 mg/kg, 40 mg/kg, 45 mg/kg, 50 mg/kg, 55 mg/kg, 60 mg/kg, 65 mg/kg, 70 mg/kg, 75 mg/kg, 80 mg/kg, 85 mg/kg, 90 mg/kg, 95 mg/kg, 100 mg/kg, 105 mg/kg, 110 mg/kg, 115 mg/kg, 120 mg/kg, 125 mg/kg, 130 mg/kg, 135 mg/kg, 140 mg/kg, 145 mg/kg, 150 mg/kg, 155 mg/kg, 160 mg/kg, 165 mg/kg, 170 mg/kg, 175 mg/kg, 180 mg/kg, 185 mg/kg, 190 mg/kg, 195 mg/kg, 200 mg/kg, and any fraction or integer in between any two of the preceding amounts.

In some embodiments, a NOS inhibitor composition comprising L-NPA and/or a salt thereof, such as, for example, L-NPA hydrochloride, may be administered as a suitable dosage form, as described herein, to treat, ameliorate, and/or reduce the severity of NSC-lung cancer or one or more symptoms thereof in an NSC-lung cancer subject. In certain embodiments, an nNOS inhibitor composition comprising L-NPA and/or a salt thereof, such as, for example, L-NPA hydrochloride may be administered to an NSC-lung cancer subject to treat, ameliorate, and/or reduce the severity of NSC-lung cancer or one or more symptoms thereof in the NSC-lung cancer subject: (i) via a route selected from the group consisting of buccally, inhalation, intraarterially, intraarticularly, intracardially, intracerebroventricularly, intradermally, intramedullary, intramuscularly, intraperitoneally, intrathecally, intravenously, intraventricularly, nasally, ophthalmically, orally, rectally, sublabially, sublingually, subcutaneously, topically, and transdermally; and/or (ii) with a frequency selected from the group consisting of four times daily, three times daily, twice daily, once daily, once every two days, once every three days, once every four days, once every five days, once every six days, once a week, once every eight days, once every nine days, once every ten days, once every two weeks, once every three weeks, and once a month; and/or (iii) at a dosage (based on subject weight) selected from the group consisting of 0.1 μg/kg, 0.5 μg/kg, 1 μg/kg, 1.5 μg/kg, 2.0 μg/kg, 2.5 μg/kg, 3.0 μg/kg, 3.5 μg/kg, 4.0 μg/kg, 4.5 μg/kg, 5.0 μg/kg, 10 μg/kg, 15 μg/kg, 20 μg/kg, 25 μg/kg, 30 μg/kg, 35 μg/kg, 40 μg/kg, 45 μg/kg, 50 μg/kg, 55 μg/kg, 60 μg/kg, 65 μg/kg, 70 μg/kg, 75 μg/kg, 80 μg/kg, 85 μg/kg, 90 μg/kg, 95 μg/kg, 100 μg/kg, 150 μg/kg, 200 μg/kg, 250 μg/kg, 300 μg/kg, 350 μg/kg, 400 μg/kg, 450 μg/kg, 500 μg/kg, 550 μg/kg, 600 μg/kg, 650 μg/kg, 700 μg/kg, 750 μg/kg, 800 μg/kg, 850 μg/kg, 900 μg/kg, 1 mg/kg, 1.5 mg/kg, 2.0 mg/kg, 2.5 mg/kg, 3 mg/kg, 3.5 mg/kg, 4.0 mg/kg, 4.5 mg/kg, 5 mg/kg, 5.5 mg/kg, 6 mg/kg, 6.5 mg/kg, 7 mg/kg, 7.5 mg/kg, 8 mg/kg, 8.5 mg/kg, 9 mg/kg, 9.5 mg/kg, 10 mg/kg 10.5 mg/kg, 11 mg/kg, 11.5 mg/kg, 12 mg/kg, 12.5 mg/kg, 13 mg/kg, 13.5 mg/kg, 14 mg/kg, 14.5 mg/kg, 15 mg/kg, 16 mg/kg, 17 mg/kg, 18 mg/kg, 19 mg/kg, 20 mg/kg, 21 mg/kg, 22 mg/kg, 23 mg/kg, 24 mg/kg, 25 mg/kg, 30 mg/kg, 35 mg/kg, 40 mg/kg, 45 mg/kg, 50 mg/kg, 55 mg/kg, 60 mg/kg, 65 mg/kg, 70 mg/kg, 75 mg/kg, 80 mg/kg, 85 mg/kg, 90 mg/kg, 95 mg/kg, 100 mg/kg, 105 mg/kg, 110 mg/kg, 115 mg/kg, 120 mg/kg, 125 mg/kg, 130 mg/kg, 135 mg/kg, 140 mg/kg, 145 mg/kg, 150 mg/kg, 155 mg/kg, 160 mg/kg, 165 mg/kg, 170 mg/kg, 175 mg/kg, 180 mg/kg, 185 mg/kg, 190 mg/kg, 195 mg/kg, 200 mg/kg, and any fraction or integer in between any two of the preceding amounts.

In certain embodiments, a NOS inhibitor composition comprising L-NIO and/or a salt thereof, such as, for example, L-NIO dihydrochloride, may be administered as a suitable dosage form, as described herein, to treat, ameliorate, and/or reduce the severity of NSC-lung cancer or one or more symptoms thereof in an NSC-lung cancer subject. In some embodiments, an nNOS inhibitor composition comprising L-NIO and/or a salt thereof, such as, for example, L-NIO dihydrochloride, may be administered to an NSC-lung cancer subject to treat, ameliorate, and/or reduce the severity of NSC-lung cancer or one or more symptoms thereof in the NSC-lung cancer subject: (i) via a route selected from the group consisting of buccally, inhalation, intraarterially, intraarticularly, intracardially, intracerebroventricularly, intradermally, intramedullary, intramuscularly, intraperitoneally, intrathecally, intravenously, intraventricularly, nasally, ophthalmically, orally, rectally, sublabially, sublingually, subcutaneously, topically, and transdermally; and/or (ii) with a frequency selected from the group consisting of four times daily, three times daily, twice daily, once daily, once every two days, once every three days, once every four days, once every five days, once every six days, once a week, once every eight days, once every nine days, once every ten days, once every two weeks, once every three weeks, and once a month; and/or (iii) at a dosage (based on subject weight) selected from the group consisting of 0.1 μg/kg, 0.5 μg/kg, 1 μg/kg, 1.5 μg/kg, 2.0 μg/kg, 2.5 μg/kg, 3.0 μg/kg, 3.5 μg/kg, 4.0 μg/kg, 4.5 μg/kg, 5.0 μg/kg, 10 μg/kg, 15 μg/kg, 20 μg/kg, 25 μg/kg, 30 μg/kg, 35 μg/kg, 40 μg/kg, 45 μg/kg, 50 μg/kg, 55 μg/kg, 60 μg/kg, 65 μg/kg, 70 μg/kg, 75 μg/kg, 80 μg/kg, 85 μg/kg, 90 μg/kg, 95 μg/kg, 100 μg/kg, 150 μg/kg, 200 μg/kg, 250 μg/kg, 300 μg/kg, 350 μg/kg, 400 μg/kg, 450 μg/kg, 500 μg/kg, 550 μg/kg, 600 μg/kg, 650 μg/kg, 700 μg/kg, 750 μg/kg, 800 μg/kg, 850 μg/kg, 900 μg/kg, 1 mg/kg, 1.5 mg/kg, 2.0 mg/kg, 2.5 mg/kg, 3 mg/kg, 3.5 mg/kg, 4.0 mg/kg, 4.5 mg/kg, 5 mg/kg, 5.5 mg/kg, 6 mg/kg, 6.5 mg/kg, 7 mg/kg, 7.5 mg/kg, 8 mg/kg, 8.5 mg/kg, 9 mg/kg, 9.5 mg/kg, 10 mg/kg 10.5 mg/kg, 11 mg/kg, 11.5 mg/kg, 12 mg/kg, 12.5 mg/kg, 13 mg/kg, 13.5 mg/kg, 14 mg/kg, 14.5 mg/kg, 15 mg/kg, 16 mg/kg, 17 mg/kg, 18 mg/kg, 19 mg/kg, 20 mg/kg, 21 mg/kg, 22 mg/kg, 23 mg/kg, 24 mg/kg, 25 mg/kg, 30 mg/kg, 35 mg/kg, 40 mg/kg, 45 mg/kg, 50 mg/kg, 55 mg/kg, 60 mg/kg, 65 mg/kg, 70 mg/kg, 75 mg/kg, 80 mg/kg, 85 mg/kg, 90 mg/kg, 95 mg/kg, 100 mg/kg, 105 mg/kg, 110 mg/kg, 115 mg/kg, 120 mg/kg, 125 mg/kg, 130 mg/kg, 135 mg/kg, 140 mg/kg, 145 mg/kg, 150 mg/kg, 155 mg/kg, 160 mg/kg, 165 mg/kg, 170 mg/kg, 175 mg/kg, 180 mg/kg, 185 mg/kg, 190 mg/kg, 195 mg/kg, 200 mg/kg, and any fraction or integer in between any two of the preceding amounts.

In some embodiments, a NOS inhibitor composition comprising ARL-17477 and/or a salt thereof, such as, for example, ARL-17477 dihydrochloride, may be administered as a suitable dosage form, as described herein, to treat, ameliorate, and/or reduce the severity of NSC-lung cancer or one or more symptoms thereof in an NSC-lung cancer subject. In certain embodiments, an nNOS inhibitor composition comprising ARL-17477 and/or a salt thereof, such as, for example, ARL-17477 dihydrochloride, may be administered to an NSC-lung cancer subject to treat, ameliorate, and/or reduce the severity of NSC-lung cancer or one or more symptoms thereof in the NSC-lung cancer subject: (i) via a route selected from the group consisting of buccally, inhalation, intraarterially, intraarticularly, intracardially, intracerebroventricularly, intradermally, intramedullary, intramuscularly, intraperitoneally, intrathecally, intravenously, intraventricularly, nasally, ophthalmically, orally, rectally, sublabially, sublingually, subcutaneously, topically, and transdermally; and/or (ii) with a frequency selected from the group consisting of four times daily, three times daily, twice daily, once daily, once every two days, once every three days, once every four days, once every five days, once every six days, once a week, once every eight days, once every nine days, once every ten days, once every two weeks, once every three weeks, and once a month; and/or (iii) at a dosage (based on subject weight) selected from the group consisting of 0.1 μg/kg, 0.5 μg/kg, 1 μg/kg, 1.5 μg/kg, 2.0 μg/kg, 2.5 μg/kg, 3.0 μg/kg, 3.5 μg/kg, 4.0 μg/kg, 4.5 μg/kg, 5.0 μg/kg, 10 μg/kg, 15 μg/kg, 20 μg/kg, 25 μg/kg, 30 μg/kg, 35 μg/kg, 40 μg/kg, 45 μg/kg, 50 μg/kg, 55 μg/kg, 60 μg/kg, 65 μg/kg, 70 μg/kg, 75 μg/kg, 80 μg/kg, 85 μg/kg, 90 μg/kg, 95 μg/kg, 100 μg/kg, 150 μg/kg, 200 μg/kg, 250 μg/kg, 300 μg/kg, 350 μg/kg, 400 μg/kg, 450 μg/kg, 500 μg/kg, 550 μg/kg, 600 μg/kg, 650 μg/kg, 700 μg/kg, 750 μg/kg, 800 μg/kg, 850 μg/kg, 900 μg/kg, 1 mg/kg, 1.5 mg/kg, 2.0 mg/kg, 2.5 mg/kg, 3 mg/kg, 3.5 mg/kg, 4.0 mg/kg, 4.5 mg/kg, 5 mg/kg, 5.5 mg/kg, 6 mg/kg, 6.5 mg/kg, 7 mg/kg, 7.5 mg/kg, 8 mg/kg, 8.5 mg/kg, 9 mg/kg, 9.5 mg/kg, 10 mg/kg 10.5 mg/kg, 11 mg/kg, 11.5 mg/kg, 12 mg/kg, 12.5 mg/kg, 13 mg/kg, 13.5 mg/kg, 14 mg/kg, 14.5 mg/kg, 15 mg/kg, 16 mg/kg, 17 mg/kg, 18 mg/kg, 19 mg/kg, 20 mg/kg, 21 mg/kg, 22 mg/kg, 23 mg/kg, 24 mg/kg, 25 mg/kg, 30 mg/kg, 35 mg/kg, 40 mg/kg, 45 mg/kg, 50 mg/kg, 55 mg/kg, 60 mg/kg, 65 mg/kg, 70 mg/kg, 75 mg/kg, 80 mg/kg, 85 mg/kg, 90 mg/kg, 95 mg/kg, 100 mg/kg, 105 mg/kg, 110 mg/kg, 115 mg/kg, 120 mg/kg, 125 mg/kg, 130 mg/kg, 135 mg/kg, 140 mg/kg, 145 mg/kg, 150 mg/kg, 155 mg/kg, 160 mg/kg, 165 mg/kg, 170 mg/kg, 175 mg/kg, 180 mg/kg, 185 mg/kg, 190 mg/kg, 195 mg/kg, 200 mg/kg, and any fraction or integer in between any two of the preceding amounts.

In certain embodiments, a NOS inhibitor composition comprising L-NNA and/or a salt thereof may be administered as a suitable dosage form, as described herein, to treat, ameliorate, and/or reduce the severity of NSC-lung cancer or one or more symptoms thereof in an NSC-lung cancer subject. In some embodiments, an nNOS inhibitor composition comprising L-NNA and/or a salt thereof may be administered to an NSC-lung cancer subject to treat, ameliorate, and/or reduce the severity of NSC-lung cancer or one or more symptoms thereof in the NSC-lung cancer subject: (i) via a route selected from the group consisting of buccally, inhalation, intraarterially, intraarticularly, intracardially, intracerebroventricularly, intradermally, intramedullary, intramuscularly, intraperitoneally, intrathecally, intravenously, intraventricularly, nasally, ophthalmically, orally, rectally, sublabially, sublingually, subcutaneously, topically, and transdermally; and/or (ii) with a frequency selected from the group consisting of four times daily, three times daily, twice daily, once daily, once every two days, once every three days, once every four days, once every five days, once every six days, once a week, once every eight days, once every nine days, once every ten days, once every two weeks, once every three weeks, and once a month; and/or (iii) at a dosage (based on subject weight) selected from the group consisting of 0.1 μg/kg, 0.5 μg/kg, 1 μg/kg, 1.5 μg/kg, 2.0 μg/kg, 2.5 μg/kg, 3.0 μg/kg, 3.5 μg/kg, 4.0 μg/kg, 4.5 μg/kg, 5.0 μg/kg, 10 μg/kg, 15 μg/kg, 20 μg/kg, 25 μg/kg, 30 μg/kg, 35 μg/kg, 40 μg/kg, 45 μg/kg, 50 μg/kg, 55 μg/kg, 60 μg/kg, 65 μg/kg, 70 μg/kg, 75 μg/kg, 80 μg/kg, 85 μg/kg, 90 μg/kg, 95 μg/kg, 100 μg/kg, 150 μg/kg, 200 μg/kg, 250 μg/kg, 300 μg/kg, 350 μg/kg, 400 μg/kg, 450 μg/kg, 500 μg/kg, 550 μg/kg, 600 μg/kg, 650 μg/kg, 700 μg/kg, 750 μg/kg, 800 μg/kg, 850 μg/kg, 900 μg/kg, 1 mg/kg, 1.5 mg/kg, 2.0 mg/kg, 2.5 mg/kg, 3 mg/kg, 3.5 mg/kg, 4.0 mg/kg, 4.5 mg/kg, 5 mg/kg, 5.5 mg/kg, 6 mg/kg, 6.5 mg/kg, 7 mg/kg, 7.5 mg/kg, 8 mg/kg, 8.5 mg/kg, 9 mg/kg, 9.5 mg/kg, 10 mg/kg 10.5 mg/kg, 11 mg/kg, 11.5 mg/kg, 12 mg/kg, 12.5 mg/kg, 13 mg/kg, 13.5 mg/kg, 14 mg/kg, 14.5 mg/kg, 15 mg/kg, 16 mg/kg, 17 mg/kg, 18 mg/kg, 19 mg/kg, 20 mg/kg, 21 mg/kg, 22 mg/kg, 23 mg/kg, 24 mg/kg, 25 mg/kg, 30 mg/kg, 35 mg/kg, 40 mg/kg, 45 mg/kg, 50 mg/kg, 55 mg/kg, 60 mg/kg, 65 mg/kg, 70 mg/kg, 75 mg/kg, 80 mg/kg, 85 mg/kg, 90 mg/kg, 95 mg/kg, 100 mg/kg, 105 mg/kg, 110 mg/kg, 115 mg/kg, 120 mg/kg, 125 mg/kg, 130 mg/kg, 135 mg/kg, 140 mg/kg, 145 mg/kg, 150 mg/kg, 155 mg/kg, 160 mg/kg, 165 mg/kg, 170 mg/kg, 175 mg/kg, 180 mg/kg, 185 mg/kg, 190 mg/kg, 195 mg/kg, 200 mg/kg, and any fraction or integer in between any two of the preceding amounts.

In some embodiments, a NOS inhibitor composition comprising L-NAME and/or a salt thereof, such as, for example, L-NAME hydrochloride, may be administered as a suitable dosage form, as described herein, to treat, ameliorate, and/or reduce the severity of NSC-lung cancer or one or more symptoms thereof in an NSC-lung cancer subject. In certain embodiments, an nNOS inhibitor composition comprising L-NAME and/or a salt thereof, such as, for example, L-NAME hydrochloride, may be administered to an NSC-lung cancer subject to treat, ameliorate, and/or reduce the severity of NSC-lung cancer or one or more symptoms thereof in the NSC-lung cancer subject: (i) via a route selected from the group consisting of buccally, inhalation, intraarterially, intraarticularly, intracardially, intracerebroventricularly, intradermally, intramedullary, intramuscularly, intraperitoneally, intrathecally, intravenously, intraventricularly, nasally, ophthalmically, orally, rectally, sublabially, sublingually, subcutaneously, topically, and transdermally; and/or (ii) with a frequency selected from the group consisting of four times daily, three times daily, twice daily, once daily, once every two days, once every three days, once every four days, once every five days, once every six days, once a week, once every eight days, once every nine days, once every ten days, once every two weeks, once every three weeks, and once a month; and/or (iii) at a dosage (based on subject weight) selected from the group consisting of 0.1 μg/kg, 0.5 μg/kg, 1 μg/kg, 1.5 μg/kg, 2.0 μg/kg, 2.5 μg/kg, 3.0 μg/kg, 3.5 μg/kg, 4.0 μg/kg, 4.5 μg/kg, 5.0 μg/kg, 10 μg/kg, 15 μg/kg, 20 μg/kg, 25 μg/kg, 30 μg/kg, 35 μg/kg, 40 μg/kg, 45 μg/kg, 50 μg/kg, 55 μg/kg, 60 μg/kg, 65 μg/kg, 70 μg/kg, 75 μg/kg, 80 μg/kg, 85 μg/kg, 90 μg/kg, 95 μg/kg, 100 μg/kg, 150 μg/kg, 200 μg/kg, 250 μg/kg, 300 μg/kg, 350 μg/kg, 400 μg/kg, 450 μg/kg, 500 μg/kg, 550 μg/kg, 600 μg/kg, 650 μg/kg, 700 μg/kg, 750 μg/kg, 800 μg/kg, 850 μg/kg, 900 μg/kg, 1 mg/kg, 1.5 mg/kg, 2.0 mg/kg, 2.5 mg/kg, 3 mg/kg, 3.5 mg/kg, 4.0 mg/kg, 4.5 mg/kg, 5 mg/kg, 5.5 mg/kg, 6 mg/kg, 6.5 mg/kg, 7 mg/kg, 7.5 mg/kg, 8 mg/kg, 8.5 mg/kg, 9 mg/kg, 9.5 mg/kg, 10 mg/kg 10.5 mg/kg, 11 mg/kg, 11.5 mg/kg, 12 mg/kg, 12.5 mg/kg, 13 mg/kg, 13.5 mg/kg, 14 mg/kg, 14.5 mg/kg, 15 mg/kg, 16 mg/kg, 17 mg/kg, 18 mg/kg, 19 mg/kg, 20 mg/kg, 21 mg/kg, 22 mg/kg, 23 mg/kg, 24 mg/kg, 25 mg/kg, 30 mg/kg, 35 mg/kg, 40 mg/kg, 45 mg/kg, 50 mg/kg, 55 mg/kg, 60 mg/kg, 65 mg/kg, 70 mg/kg, 75 mg/kg, 80 mg/kg, 85 mg/kg, 90 mg/kg, 95 mg/kg, 100 mg/kg, 105 mg/kg, 110 mg/kg, 115 mg/kg, 120 mg/kg, 125 mg/kg, 130 mg/kg, 135 mg/kg, 140 mg/kg, 145 mg/kg, 150 mg/kg, 155 mg/kg, 160 mg/kg, 165 mg/kg, 170 mg/kg, 175 mg/kg, 180 mg/kg, 185 mg/kg, 190 mg/kg, 195 mg/kg, 200 mg/kg, and any fraction or integer in between any two of the preceding amounts.

In certain embodiments, a NOS inhibitor composition comprising L-NMMA and/or a salt thereof, such as, for example, L-NMMA acetate, may be administered as a suitable dosage form, as described herein, to treat, ameliorate, and/or reduce the severity of NSC-lung cancer or one or more symptoms thereof in an NSC-lung cancer subject. In some embodiments, an nNOS inhibitor composition comprising L-NMMA and/or a salt thereof, such as, for example, L-NMMA acetate, may be administered to an NSC-lung cancer subject to treat, ameliorate, and/or reduce the severity of NSC-lung cancer or one or more symptoms thereof in the NSC-lung cancer subject: (i) via a route selected from the group consisting of buccally, inhalation, intraarterially, intraarticularly, intracardially, intracerebroventricularly, intradermally, intramedullary, intramuscularly, intraperitoneally, intrathecally, intravenously, intraventricularly, nasally, ophthalmically, orally, rectally, sublabially, sublingually, subcutaneously, topically, and transdermally; and/or (ii) with a frequency selected from the group consisting of four times daily, three times daily, twice daily, once daily, once every two days, once every three days, once every four days, once every five days, once every six days, once a week, once every eight days, once every nine days, once every ten days, once every two weeks, once every three weeks, and once a month; and/or (iii) at a dosage (based on subject weight) selected from the group consisting of 0.1 μg/kg, 0.5 μg/kg, 1 μg/kg, 1.5 μg/kg, 2.0 μg/kg, 2.5 μg/kg, 3.0 μg/kg, 3.5 μg/kg, 4.0 μg/kg, 4.5 μg/kg, 5.0 μg/kg, 10 μg/kg, 15 μg/kg, 20 μg/kg, 25 μg/kg, 30 μg/kg, 35 μg/kg, 40 μg/kg, 45 μg/kg, 50 μg/kg, 55 μg/kg, 60 μg/kg, 65 μg/kg, 70 μg/kg, 75 μg/kg, 80 μg/kg, 85 μg/kg, 90 μg/kg, 95 μg/kg, 100 μg/kg, 150 μg/kg, 200 μg/kg, 250 μg/kg, 300 μg/kg, 350 μg/kg, 400 μg/kg, 450 μg/kg, 500 μg/kg, 550 μg/kg, 600 μg/kg, 650 μg/kg, 700 μg/kg, 750 μg/kg, 800 μg/kg, 850 μg/kg, 900 μg/kg, 1 mg/kg, 1.5 mg/kg, 2.0 mg/kg, 2.5 mg/kg, 3 mg/kg, 3.5 mg/kg, 4.0 mg/kg, 4.5 mg/kg, 5 mg/kg, 5.5 mg/kg, 6 mg/kg, 6.5 mg/kg, 7 mg/kg, 7.5 mg/kg, 8 mg/kg, 8.5 mg/kg, 9 mg/kg, 9.5 mg/kg, 10 mg/kg 10.5 mg/kg, 11 mg/kg, 11.5 mg/kg, 12 mg/kg, 12.5 mg/kg, 13 mg/kg, 13.5 mg/kg, 14 mg/kg, 14.5 mg/kg, 15 mg/kg, 16 mg/kg, 17 mg/kg, 18 mg/kg, 19 mg/kg, 20 mg/kg, 21 mg/kg, 22 mg/kg, 23 mg/kg, 24 mg/kg, 25 mg/kg, 30 mg/kg, 35 mg/kg, 40 mg/kg, 45 mg/kg, 50 mg/kg, 55 mg/kg, 60 mg/kg, 65 mg/kg, 70 mg/kg, 75 mg/kg, 80 mg/kg, 85 mg/kg, 90 mg/kg, 95 mg/kg, 100 mg/kg, 105 mg/kg, 110 mg/kg, 115 mg/kg, 120 mg/kg, 125 mg/kg, 130 mg/kg, 135 mg/kg, 140 mg/kg, 145 mg/kg, 150 mg/kg, 155 mg/kg, 160 mg/kg, 165 mg/kg, 170 mg/kg, 175 mg/kg, 180 mg/kg, 185 mg/kg, 190 mg/kg, 195 mg/kg, 200 mg/kg, and any fraction or integer in between any two of the preceding amounts.

In some embodiments, a NOS inhibitor composition comprising GW-274150 and/or a salt thereof, such as, for example, GW-274150 phosphate, may be administered as a suitable dosage form, as described herein, to treat, ameliorate, and/or reduce the severity of NSC-lung cancer or one or more symptoms thereof in an NSC-lung cancer subject. In certain embodiments, an nNOS inhibitor composition comprising GW-274150 and/or a salt thereof, such as, for example, GW-274150 phosphate, may be administered to an NSC-lung cancer subject to treat, ameliorate, and/or reduce the severity of NSC-lung cancer or one or more symptoms thereof in the NSC-lung cancer subject: (i) via a route selected from the group consisting of buccally, inhalation, intraarterially, intraarticularly, intracardially, intracerebroventricularly, intradermally, intramedullary, intramuscularly, intraperitoneally, intrathecally, intravenously, intraventricularly, nasally, ophthalmically, orally, rectally, sublabially, sublingually, subcutaneously, topically, and transdermally; and/or (ii) with a frequency selected from the group consisting of four times daily, three times daily, twice daily, once daily, once every two days, once every three days, once every four days, once every five days, once every six days, once a week, once every eight days, once every nine days, once every ten days, once every two weeks, once every three weeks, and once a month; and/or (iii) at a dosage (based on subject weight) selected from the group consisting of 0.1 μg/kg, 0.5 μg/kg, 1 μg/kg, 1.5 μg/kg, 2.0 μg/kg, 2.5 μg/kg, 3.0 μg/kg, 3.5 μg/kg, 4.0 μg/kg, 4.5 μg/kg, 5.0 μg/kg, 10 μg/kg, 15 μg/kg, 20 μg/kg, 25 μg/kg, 30 μg/kg, 35 μg/kg, 40 μg/kg, 45 μg/kg, 50 μg/kg, 55 μg/kg, 60 μg/kg, 65 μg/kg, 70 μg/kg, 75 μg/kg, 80 μg/kg, 85 μg/kg, 90 μg/kg, 95 μg/kg, 100 μg/kg, 150 μg/kg, 200 μg/kg, 250 μg/kg, 300 μg/kg, 350 μg/kg, 400 μg/kg, 450 μg/kg, 500 μg/kg, 550 μg/kg, 600 μg/kg, 650 μg/kg, 700 μg/kg, 750 μg/kg, 800 μg/kg, 850 μg/kg, 900 μg/kg, 1 mg/kg, 1.5 mg/kg, 2.0 mg/kg, 2.5 mg/kg, 3 mg/kg, 3.5 mg/kg, 4.0 mg/kg, 4.5 mg/kg, 5 mg/kg, 5.5 mg/kg, 6 mg/kg, 6.5 mg/kg, 7 mg/kg, 7.5 mg/kg, 8 mg/kg, 8.5 mg/kg, 9 mg/kg, 9.5 mg/kg, 10 mg/kg 10.5 mg/kg, 11 mg/kg, 11.5 mg/kg, 12 mg/kg, 12.5 mg/kg, 13 mg/kg, 13.5 mg/kg, 14 mg/kg, 14.5 mg/kg, 15 mg/kg, 16 mg/kg, 17 mg/kg, 18 mg/kg, 19 mg/kg, 20 mg/kg, 21 mg/kg, 22 mg/kg, 23 mg/kg, 24 mg/kg, 25 mg/kg, 30 mg/kg, 35 mg/kg, 40 mg/kg, 45 mg/kg, 50 mg/kg, 55 mg/kg, 60 mg/kg, 65 mg/kg, 70 mg/kg, 75 mg/kg, 80 mg/kg, 85 mg/kg, 90 mg/kg, 95 mg/kg, 100 mg/kg, 105 mg/kg, 110 mg/kg, 115 mg/kg, 120 mg/kg, 125 mg/kg, 130 mg/kg, 135 mg/kg, 140 mg/kg, 145 mg/kg, 150 mg/kg, 155 mg/kg, 160 mg/kg, 165 mg/kg, 170 mg/kg, 175 mg/kg, 180 mg/kg, 185 mg/kg, 190 mg/kg, 195 mg/kg, 200 mg/kg, and any fraction or integer in between any two of the preceding amounts.

In certain embodiments, a NOS inhibitor composition comprising L-NIL and/or a salt thereof, such as, for example, L-NIL hydrochloride, may be administered as a suitable dosage form, as described herein, to treat, ameliorate, and/or reduce the severity of NSC-lung cancer or one or more symptoms thereof in an NSC-lung cancer subject. In some embodiments, an nNOS inhibitor composition comprising L-NIL and/or a salt thereof, such as, for example, L-NIL hydrochloride, may be administered to an NSC-lung cancer subject to treat, ameliorate, and/or reduce the severity of NSC-lung cancer or one or more symptoms thereof in the NSC-lung cancer subject: (i) via a route selected from the group consisting of buccally, inhalation, intraarterially, intraarticularly, intracardially, intracerebroventricularly, intradermally, intramedullary, intramuscularly, intraperitoneally, intrathecally, intravenously, intraventricularly, nasally, ophthalmically, orally, rectally, sublabially, sublingually, subcutaneously, topically, and transdermally; and/or (ii) with a frequency selected from the group consisting of four times daily, three times daily, twice daily, once daily, once every two days, once every three days, once every four days, once every five days, once every six days, once a week, once every eight days, once every nine days, once every ten days, once every two weeks, once every three weeks, and once a month; and/or (iii) at a dosage (based on subject weight) selected from the group consisting of 0.1 μg/kg, 0.5 μg/kg, 1 μg/kg, 1.5 μg/kg, 2.0 μg/kg, 2.5 μg/kg, 3.0 μg/kg, 3.5 μg/kg, 4.0 μg/kg, 4.5 μg/kg, 5.0 μg/kg, 10 μg/kg, 15 μg/kg, 20 μg/kg, 25 μg/kg, 30 μg/kg, 35 μg/kg, 40 μg/kg, 45 μg/kg, 50 μg/kg, 55 μg/kg, 60 μg/kg, 65 μg/kg, 70 μg/kg, 75 μg/kg, 80 μg/kg, 85 μg/kg, 90 μg/kg, 95 μg/kg, 100 μg/kg, 150 μg/kg, 200 μg/kg, 250 μg/kg, 300 μg/kg, 350 μg/kg, 400 μg/kg, 450 μg/kg, 500 μg/kg, 550 μg/kg, 600 μg/kg, 650 μg/kg, 700 μg/kg, 750 μg/kg, 800 μg/kg, 850 μg/kg, 900 μg/kg, 1 mg/kg, 1.5 mg/kg, 2.0 mg/kg, 2.5 mg/kg, 3 mg/kg, 3.5 mg/kg, 4.0 mg/kg, 4.5 mg/kg, 5 mg/kg, 5.5 mg/kg, 6 mg/kg, 6.5 mg/kg, 7 mg/kg, 7.5 mg/kg, 8 mg/kg, 8.5 mg/kg, 9 mg/kg, 9.5 mg/kg, 10 mg/kg 10.5 mg/kg, 11 mg/kg, 11.5 mg/kg, 12 mg/kg, 12.5 mg/kg, 13 mg/kg, 13.5 mg/kg, 14 mg/kg, 14.5 mg/kg, 15 mg/kg, 16 mg/kg, 17 mg/kg, 18 mg/kg, 19 mg/kg, 20 mg/kg, 21 mg/kg, 22 mg/kg, 23 mg/kg, 24 mg/kg, 25 mg/kg, 30 mg/kg, 35 mg/kg, 40 mg/kg, 45 mg/kg, 50 mg/kg, 55 mg/kg, 60 mg/kg, 65 mg/kg, 70 mg/kg, 75 mg/kg, 80 mg/kg, 85 mg/kg, 90 mg/kg, 95 mg/kg, 100 mg/kg, 105 mg/kg, 110 mg/kg, 115 mg/kg, 120 mg/kg, 125 mg/kg, 130 mg/kg, 135 mg/kg, 140 mg/kg, 145 mg/kg, 150 mg/kg, 155 mg/kg, 160 mg/kg, 165 mg/kg, 170 mg/kg, 175 mg/kg, 180 mg/kg, 185 mg/kg, 190 mg/kg, 195 mg/kg, 200 mg/kg, and any fraction or integer in between any two of the preceding amounts.

In some embodiments, a NOS inhibitor composition comprising 1400W and/or a salt thereof, such as, for example, 1400W dihydrochloride, may be administered as a suitable dosage form, as described herein, to treat, ameliorate, and/or reduce the severity of NSC-lung cancer or one or more symptoms thereof in an NSC-lung cancer subject. In certain embodiments, an nNOS inhibitor composition comprising 1400W and/or a salt thereof, such as, for example, 1400W dihydrochloride, may be administered to an NSC-lung cancer subject to treat, ameliorate, and/or reduce the severity of NSC-lung cancer or one or more symptoms thereof in the NSC-lung cancer subject: (i) via a route selected from the group consisting of buccally, inhalation, intraarterially, intraarticularly, intracardially, intracerebroventricularly, intradermally, intramedullary, intramuscularly, intraperitoneally, intrathecally, intravenously, intraventricularly, nasally, ophthalmically, orally, rectally, sublabially, sublingually, subcutaneously, topically, and transdermally; and/or (ii) with a frequency selected from the group consisting of four times daily, three times daily, twice daily, once daily, once every two days, once every three days, once every four days, once every five days, once every six days, once a week, once every eight days, once every nine days, once every ten days, once every two weeks, once every three weeks, and once a month; and/or (iii) at a dosage (based on subject weight) selected from the group consisting of 0.1 μg/kg, 0.5 μg/kg, 1 μg/kg, 1.5 μg/kg, 2.0 μg/kg, 2.5 μg/kg, 3.0 μg/kg, 3.5 μg/kg, 4.0 μg/kg, 4.5 μg/kg, 5.0 μg/kg, 10 μg/kg, 15 μg/kg, 20 μg/kg, 25 μg/kg, 30 μg/kg, 35 μg/kg, 40 μg/kg, 45 μg/kg, 50 μg/kg, 55 μg/kg, 60 μg/kg, 65 μg/kg, 70 μg/kg, 75 μg/kg, 80 μg/kg, 85 μg/kg, 90 μg/kg, 95 μg/kg, 100 μg/kg, 150 μg/kg, 200 μg/kg, 250 μg/kg, 300 μg/kg, 350 μg/kg, 400 μg/kg, 450 μg/kg, 500 μg/kg, 550 μg/kg, 600 μg/kg, 650 μg/kg, 700 μg/kg, 750 μg/kg, 800 μg/kg, 850 μg/kg, 900 μg/kg, 1 mg/kg, 1.5 mg/kg, 2.0 mg/kg, 2.5 mg/kg, 3 mg/kg, 3.5 mg/kg, 4.0 mg/kg, 4.5 mg/kg, 5 mg/kg, 5.5 mg/kg, 6 mg/kg, 6.5 mg/kg, 7 mg/kg, 7.5 mg/kg, 8 mg/kg, 8.5 mg/kg, 9 mg/kg, 9.5 mg/kg, 10 mg/kg 10.5 mg/kg, 11 mg/kg, 11.5 mg/kg, 12 mg/kg, 12.5 mg/kg, 13 mg/kg, 13.5 mg/kg, 14 mg/kg, 14.5 mg/kg, 15 mg/kg, 16 mg/kg, 17 mg/kg, 18 mg/kg, 19 mg/kg, 20 mg/kg, 21 mg/kg, 22 mg/kg, 23 mg/kg, 24 mg/kg, 25 mg/kg, 30 mg/kg, 35 mg/kg, 40 mg/kg, 45 mg/kg, 50 mg/kg, 55 mg/kg, 60 mg/kg, 65 mg/kg, 70 mg/kg, 75 mg/kg, 80 mg/kg, 85 mg/kg, 90 mg/kg, 95 mg/kg, 100 mg/kg, 105 mg/kg, 110 mg/kg, 115 mg/kg, 120 mg/kg, 125 mg/kg, 130 mg/kg, 135 mg/kg, 140 mg/kg, 145 mg/kg, 150 mg/kg, 155 mg/kg, 160 mg/kg, 165 mg/kg, 170 mg/kg, 175 mg/kg, 180 mg/kg, 185 mg/kg, 190 mg/kg, 195 mg/kg, 200 mg/kg, and any fraction or integer in between any two of the preceding amounts.

In certain embodiments, a NOS inhibitor composition comprising OKN-007 and/or a salt thereof, such as, for example, OKN-007 sodium, may be administered as a suitable dosage form, as described herein, to treat, ameliorate, and/or reduce the severity of NSC-lung cancer or one or more symptoms thereof in an NSC-lung cancer subject. In some embodiments, an nNOS inhibitor composition comprising OKN-007 and/or a salt thereof, such as, for example, OKN-007 sodium, may be administered to an NSC-lung cancer subject to treat, ameliorate, and/or reduce the severity of NSC-lung cancer or one or more symptoms thereof in the NSC-lung cancer subject: (i) via a route selected from the group consisting of buccally, inhalation, intraarterially, intraarticularly, intracardially, intracerebroventricularly, intradermally, intramedullary, intramuscularly, intraperitoneally, intrathecally, intravenously, intraventricularly, nasally, ophthalmically, orally, rectally, sublabially, sublingually, subcutaneously, topically, and transdermally; and/or (ii) with a frequency selected from the group consisting of four times daily, three times daily, twice daily, once daily, once every two days, once every three days, once every four days, once every five days, once every six days, once a week, once every eight days, once every nine days, once every ten days, once every two weeks, once every three weeks, and once a month; and/or (iii) at a dosage (based on subject weight) selected from the group consisting of 0.1 μg/kg, 0.5 μg/kg, 1 μg/kg, 1.5 μg/kg, 2.0 μg/kg, 2.5 μg/kg, 3.0 μg/kg, 3.5 μg/kg, 4.0 μg/kg, 4.5 μg/kg, 5.0 μg/kg, 10 μg/kg, 15 μg/kg, 20 μg/kg, 25 μg/kg, 30 μg/kg, 35 μg/kg, 40 μg/kg, 45 μg/kg, 50 μg/kg, 55 μg/kg, 60 μg/kg, 65 μg/kg, 70 μg/kg, 75 μg/kg, 80 μg/kg, 85 μg/kg, 90 μg/kg, 95 μg/kg, 100 μg/kg, 150 μg/kg, 200 μg/kg, 250 μg/kg, 300 μg/kg, 350 μg/kg, 400 μg/kg, 450 μg/kg, 500 μg/kg, 550 μg/kg, 600 μg/kg, 650 μg/kg, 700 μg/kg, 750 μg/kg, 800 μg/kg, 850 μg/kg, 900 μg/kg, 1 mg/kg, 1.5 mg/kg, 2.0 mg/kg, 2.5 mg/kg, 3 mg/kg, 3.5 mg/kg, 4.0 mg/kg, 4.5 mg/kg, 5 mg/kg, 5.5 mg/kg, 6 mg/kg, 6.5 mg/kg, 7 mg/kg, 7.5 mg/kg, 8 mg/kg, 8.5 mg/kg, 9 mg/kg, 9.5 mg/kg, 10 mg/kg 10.5 mg/kg, 11 mg/kg, 11.5 mg/kg, 12 mg/kg, 12.5 mg/kg, 13 mg/kg, 13.5 mg/kg, 14 mg/kg, 14.5 mg/kg, 15 mg/kg, 16 mg/kg, 17 mg/kg, 18 mg/kg, 19 mg/kg, 20 mg/kg, 21 mg/kg, 22 mg/kg, 23 mg/kg, 24 mg/kg, 25 mg/kg, 30 mg/kg, 35 mg/kg, 40 mg/kg, 45 mg/kg, 50 mg/kg, 55 mg/kg, 60 mg/kg, 65 mg/kg, 70 mg/kg, 75 mg/kg, 80 mg/kg, 85 mg/kg, 90 mg/kg, 95 mg/kg, 100 mg/kg, 105 mg/kg, 110 mg/kg, 115 mg/kg, 120 mg/kg, 125 mg/kg, 130 mg/kg, 135 mg/kg, 140 mg/kg, 145 mg/kg, 150 mg/kg, 155 mg/kg, 160 mg/kg, 165 mg/kg, 170 mg/kg, 175 mg/kg, 180 mg/kg, 185 mg/kg, 190 mg/kg, 195 mg/kg, 200 mg/kg, and any fraction or integer in between any two of the preceding amounts.

In some embodiments, a NOS inhibitor composition comprising BYK-191023 and/or a salt thereof, such as, for example, BYK-191023 dihydrochloride, may be administered as a suitable dosage form, as described herein, to treat, ameliorate, and/or reduce the severity of NSC-lung cancer or one or more symptoms thereof in an NSC-lung cancer subject. In certain embodiments, an nNOS inhibitor composition comprising BYK-191023 and/or a salt thereof, such as, for example, BYK-191023 dihydrochloride, may be administered to an NSC-lung cancer subject to treat, ameliorate, and/or reduce the severity of NSC-lung cancer or one or more symptoms thereof in the NSC-lung cancer subject: (i) via a route selected from the group consisting of buccally, inhalation, intraarterially, intraarticularly, intracardially, intracerebroventricularly, intradermally, intramedullary, intramuscularly, intraperitoneally, intrathecally, intravenously, intraventricularly, nasally, ophthalmically, orally, rectally, sublabially, sublingually, subcutaneously, topically, and transdermally; and/or (ii) with a frequency selected from the group consisting of four times daily, three times daily, twice daily, once daily, once every two days, once every three days, once every four days, once every five days, once every six days, once a week, once every eight days, once every nine days, once every ten days, once every two weeks, once every three weeks, and once a month; and/or (iii) at a dosage (based on subject weight) selected from the group consisting of 0.1 μg/kg, 0.5 μg/kg, 1 μg/kg, 1.5 μg/kg, 2.0 μg/kg, 2.5 μg/kg, 3.0 μg/kg, 3.5 μg/kg, 4.0 μg/kg, 4.5 μg/kg, 5.0 μg/kg, 10 μg/kg, 15 μg/kg, 20 μg/kg, 25 μg/kg, 30 μg/kg, 35 μg/kg, 40 μg/kg, 45 μg/kg, 50 μg/kg, 55 μg/kg, 60 μg/kg, 65 μg/kg, 70 μg/kg, 75 μg/kg, 80 μg/kg, 85 μg/kg, 90 μg/kg, 95 μg/kg, 100 μg/kg, 150 μg/kg, 200 μg/kg, 250 μg/kg, 300 μg/kg, 350 μg/kg, 400 μg/kg, 450 μg/kg, 500 μg/kg, 550 μg/kg, 600 μg/kg, 650 μg/kg, 700 μg/kg, 750 μg/kg, 800 μg/kg, 850 μg/kg, 900 μg/kg, 1 mg/kg, 1.5 mg/kg, 2.0 mg/kg, 2.5 mg/kg, 3 mg/kg, 3.5 mg/kg, 4.0 mg/kg, 4.5 mg/kg, 5 mg/kg, 5.5 mg/kg, 6 mg/kg, 6.5 mg/kg, 7 mg/kg, 7.5 mg/kg, 8 mg/kg, 8.5 mg/kg, 9 mg/kg, 9.5 mg/kg, 10 mg/kg 10.5 mg/kg, 11 mg/kg, 11.5 mg/kg, 12 mg/kg, 12.5 mg/kg, 13 mg/kg, 13.5 mg/kg, 14 mg/kg, 14.5 mg/kg, 15 mg/kg, 16 mg/kg, 17 mg/kg, 18 mg/kg, 19 mg/kg, 20 mg/kg, 21 mg/kg, 22 mg/kg, 23 mg/kg, 24 mg/kg, 25 mg/kg, 30 mg/kg, 35 mg/kg, 40 mg/kg, 45 mg/kg, 50 mg/kg, 55 mg/kg, 60 mg/kg, 65 mg/kg, 70 mg/kg, 75 mg/kg, 80 mg/kg, 85 mg/kg, 90 mg/kg, 95 mg/kg, 100 mg/kg, 105 mg/kg, 110 mg/kg, 115 mg/kg, 120 mg/kg, 125 mg/kg, 130 mg/kg, 135 mg/kg, 140 mg/kg, 145 mg/kg, 150 mg/kg, 155 mg/kg, 160 mg/kg, 165 mg/kg, 170 mg/kg, 175 mg/kg, 180 mg/kg, 185 mg/kg, 190 mg/kg, 195 mg/kg, 200 mg/kg, and any fraction or integer in between any two of the preceding amounts.

In certain embodiments, a NOS inhibitor composition comprising CM544 and/or a salt thereof, such as, for example, CM544 dihydrochloride, may be administered as a suitable dosage form, as described herein, to treat, ameliorate, and/or reduce the severity of NSC-lung cancer or one or more symptoms thereof in an NSC-lung cancer subject. In some embodiments, an nNOS inhibitor composition comprising CM544 and/or a salt thereof, such as, for example, CM544 dihydrochloride, may be administered to an NSC-lung cancer subject to treat, ameliorate, and/or reduce the severity of NSC-lung cancer or one or more symptoms thereof in the NSC-lung cancer subject: (i) via a route selected from the group consisting of buccally, inhalation, intraarterially, intraarticularly, intracardially, intracerebroventricularly, intradermally, intramedullary, intramuscularly, intraperitoneally, intrathecally, intravenously, intraventricularly, nasally, ophthalmically, orally, rectally, sublabially, sublingually, subcutaneously, topically, and transdermally; and/or (ii) with a frequency selected from the group consisting of four times daily, three times daily, twice daily, once daily, once every two days, once every three days, once every four days, once every five days, once every six days, once a week, once every eight days, once every nine days, once every ten days, once every two weeks, once every three weeks, and once a month; and/or (iii) at a dosage (based on subject weight) selected from the group consisting of 0.1 μg/kg, 0.5 μg/kg, 1 μg/kg, 1.5 μg/kg, 2.0 μg/kg, 2.5 μg/kg, 3.0 μg/kg, 3.5 μg/kg, 4.0 μg/kg, 4.5 μg/kg, 5.0 μg/kg, 10 μg/kg, 15 μg/kg, 20 μg/kg, 25 μg/kg, 30 μg/kg, 35 μg/kg, 40 μg/kg, 45 μg/kg, 50 μg/kg, 55 μg/kg, 60 μg/kg, 65 μg/kg, 70 μg/kg, 75 μg/kg, 80 μg/kg, 85 μg/kg, 90 μg/kg, 95 μg/kg, 100 μg/kg, 150 μg/kg, 200 μg/kg, 250 μg/kg, 300 μg/kg, 350 μg/kg, 400 μg/kg, 450 μg/kg, 500 μg/kg, 550 μg/kg, 600 μg/kg, 650 μg/kg, 700 μg/kg, 750 μg/kg, 800 μg/kg, 850 μg/kg, 900 μg/kg, 1 mg/kg, 1.5 mg/kg, 2.0 mg/kg, 2.5 mg/kg, 3 mg/kg, 3.5 mg/kg, 4.0 mg/kg, 4.5 mg/kg, 5 mg/kg, 5.5 mg/kg, 6 mg/kg, 6.5 mg/kg, 7 mg/kg, 7.5 mg/kg, 8 mg/kg, 8.5 mg/kg, 9 mg/kg, 9.5 mg/kg, 10 mg/kg 10.5 mg/kg, 11 mg/kg, 11.5 mg/kg, 12 mg/kg, 12.5 mg/kg, 13 mg/kg, 13.5 mg/kg, 14 mg/kg, 14.5 mg/kg, 15 mg/kg, 16 mg/kg, 17 mg/kg, 18 mg/kg, 19 mg/kg, 20 mg/kg, 21 mg/kg, 22 mg/kg, 23 mg/kg, 24 mg/kg, 25 mg/kg, 30 mg/kg, 35 mg/kg, 40 mg/kg, 45 mg/kg, 50 mg/kg, 55 mg/kg, 60 mg/kg, 65 mg/kg, 70 mg/kg, 75 mg/kg, 80 mg/kg, 85 mg/kg, 90 mg/kg, 95 mg/kg, 100 mg/kg, 105 mg/kg, 110 mg/kg, 115 mg/kg, 120 mg/kg, 125 mg/kg, 130 mg/kg, 135 mg/kg, 140 mg/kg, 145 mg/kg, 150 mg/kg, 155 mg/kg, 160 mg/kg, 165 mg/kg, 170 mg/kg, 175 mg/kg, 180 mg/kg, 185 mg/kg, 190 mg/kg, 195 mg/kg, 200 mg/kg, and any fraction or integer in between any two of the preceding amounts.

In certain embodiments, an nNOS inhibitor composition comprising one or more dsRNAs each comprising at least 30 base pairs, may be administered as a suitable dosage form, as described herein, to treat, ameliorate, and/or reduce the severity of NSC-lung cancer or one or more symptoms thereof in an NSC-lung cancer subject. In some embodiments, an nNOS inhibitor composition comprising one or more dsRNAs, each comprising at least 30 base pairs, may be administered to an NSC-lung cancer subject to treat, ameliorate, and/or reduce the severity of NSC-lung cancer or one or more symptoms thereof in the NSC-lung cancer subject: (i) via a route selected from the group consisting of buccally, inhalation, intraarterially, intraarticularly, intracardially, intracerebroventricularly, intradermally, intramedullary, intramuscularly, intraperitoneally, intrathecally, intravenously, intraventricularly, nasally, ophthalmically, orally, rectally, sublabially, sublingually, subcutaneously, topically, and transdermally; and/or (ii) with a frequency selected from the group consisting of four times daily, three times daily, twice daily, once daily, once every two days, once every three days, once every four days, once every five days, once every six days, once a week, once every eight days, once every nine days, once every ten days, once every two weeks, once every three weeks, and once a month; and/or (iii) at a dosage (based on subject weight) selected from the group consisting of 0.1 μg/kg, 0.5 μg/kg, 1 μg/kg, 1.5 μg/kg, 2.0 μg/kg, 2.5 μg/kg, 3.0 μg/kg, 3.5 μg/kg, 4.0 μg/kg, 4.5 μg/kg, 5.0 μg/kg, 10 μg/kg, 15 μg/kg, 20 μg/kg, 25 μg/kg, 30 μg/kg, 35 μg/kg, 40 μg/kg, 45 μg/kg, 50 μg/kg, 55 μg/kg, 60 μg/kg, 65 μg/kg, 70 μg/kg, 75 μg/kg, 80 μg/kg, 85 μg/kg, 90 μg/kg, 95 μg/kg, 100 μg/kg, 150 μg/kg, 200 μg/kg, 250 μg/kg, 300 μg/kg, 350 μg/kg, 400 μg/kg, 450 μg/kg, 500 μg/kg, 550 μg/kg, 600 μg/kg, 650 μg/kg, 700 μg/kg, 750 μg/kg, 800 μg/kg, 850 μg/kg, 900 μg/kg, 1 mg/kg, 1.5 mg/kg, 2.0 mg/kg, 2.5 mg/kg, 3 mg/kg, 3.5 mg/kg, 4.0 mg/kg, 4.5 mg/kg, 5 mg/kg, 5.5 mg/kg, 6 mg/kg, 6.5 mg/kg, 7 mg/kg, 7.5 mg/kg, 8 mg/kg, 8.5 mg/kg, 9 mg/kg, 9.5 mg/kg, 10 mg/kg 10.5 mg/kg, 11 mg/kg, 11.5 mg/kg, 12 mg/kg, 12.5 mg/kg, 13 mg/kg, 13.5 mg/kg, 14 mg/kg, 14.5 mg/kg, 15 mg/kg, 16 mg/kg, 17 mg/kg, 18 mg/kg, 19 mg/kg, 20 mg/kg, 21 mg/kg, 22 mg/kg, 23 mg/kg, 24 mg/kg, 25 mg/kg, 30 mg/kg, 35 mg/kg, 40 mg/kg, 45 mg/kg, 50 mg/kg, 55 mg/kg, 60 mg/kg, 65 mg/kg, 70 mg/kg, 75 mg/kg, 80 mg/kg, 85 mg/kg, 90 mg/kg, 95 mg/kg, 100 mg/kg, 105 mg/kg, 110 mg/kg, 115 mg/kg, 120 mg/kg, 125 mg/kg, 130 mg/kg, 135 mg/kg, 140 mg/kg, 145 mg/kg, 150 mg/kg, 155 mg/kg, 160 mg/kg, 165 mg/kg, 170 mg/kg, 175 mg/kg, 180 mg/kg, 185 mg/kg, 190 mg/kg, 195 mg/kg, 200 mg/kg, and any fraction or integer in between any two of the preceding amounts.

In some embodiments, an nNOS inhibitor composition comprising one or more siRNAs, may be administered as a suitable dosage form, as described herein, to treat, ameliorate, and/or reduce the severity of NSC-lung cancer or one or more symptoms thereof in an NSC-lung cancer subject. In certain embodiments, an nNOS inhibitor composition comprising one or more siRNAs selected from the group consisting of SEQ ID NO. 4, SEQ ID NO. 5, SEQ ID NO. 6, SEQ ID NO. 7, SEQ ID NO. 8, and any combination thereof, may be administered as a suitable dosage form, as described herein, to treat, ameliorate, and/or reduce the severity of NSC-lung cancer or one or more symptoms thereof in an NSC-lung cancer subject. In some embodiments, an nNOS inhibitor composition comprising one or more siRNAs selected from the group consisting of SEQ ID NO. 4, SEQ ID NO. 5, SEQ ID NO. 6, SEQ ID NO. 7, SEQ ID NO. 8, and any combination thereof, may be administered to an NSC-lung cancer subject to treat, ameliorate, and/or reduce the severity of NSC-lung cancer or one or more symptoms thereof in the NSC-lung cancer subject: (i) via a route selected from the group consisting of buccally, inhalation, intraarterially, intraarticularly, intracardially, intracerebroventricularly, intradermally, intramedullary, intramuscularly, intraperitoneally, intrathecally, intravenously, intraventricularly, nasally, ophthalmically, orally, rectally, sublabially, sublingually, subcutaneously, topically, and transdermally; and/or (ii) with a frequency selected from the group consisting of four times daily, three times daily, twice daily, once daily, once every two days, once every three days, once every four days, once every five days, once every six days, once a week, once every eight days, once every nine days, once every ten days, once every two weeks, once every three weeks, and once a month; and/or (iii) at a dosage (based on subject weight) selected from the group consisting of 0.1 μg/kg, 0.5 μg/kg, 1 μg/kg, 1.5 μg/kg, 2.0 μg/kg, 2.5 μg/kg, 3.0 μg/kg, 3.5 μg/kg, 4.0 μg/kg, 4.5 μg/kg, 5.0 μg/kg, 10 μg/kg, 15 μg/kg, 20 μg/kg, 25 μg/kg, 30 μg/kg, 35 μg/kg, 40 μg/kg, 45 μg/kg, 50 μg/kg, 55 μg/kg, 60 μg/kg, 65 μg/kg, 70 μg/kg, 75 μg/kg, 80 μg/kg, 85 μg/kg, 90 μg/kg, 95 μg/kg, 100 μg/kg, 150 μg/kg, 200 μg/kg, 250 μg/kg, 300 μg/kg, 350 μg/kg, 400 μg/kg, 450 μg/kg, 500 μg/kg, 550 μg/kg, 600 μg/kg, 650 μg/kg, 700 μg/kg, 750 μg/kg, 800 μg/kg, 850 μg/kg, 900 μg/kg, 1 mg/kg, 1.5 mg/kg, 2.0 mg/kg, 2.5 mg/kg, 3 mg/kg, 3.5 mg/kg, 4.0 mg/kg, 4.5 mg/kg, 5 mg/kg, 5.5 mg/kg, 6 mg/kg, 6.5 mg/kg, 7 mg/kg, 7.5 mg/kg, 8 mg/kg, 8.5 mg/kg, 9 mg/kg, 9.5 mg/kg, 10 mg/kg 10.5 mg/kg, 11 mg/kg, 11.5 mg/kg, 12 mg/kg, 12.5 mg/kg, 13 mg/kg, 13.5 mg/kg, 14 mg/kg, 14.5 mg/kg, 15 mg/kg, 16 mg/kg, 17 mg/kg, 18 mg/kg, 19 mg/kg, 20 mg/kg, 21 mg/kg, 22 mg/kg, 23 mg/kg, 24 mg/kg, 25 mg/kg, 30 mg/kg, 35 mg/kg, 40 mg/kg, 45 mg/kg, 50 mg/kg, 55 mg/kg, 60 mg/kg, 65 mg/kg, 70 mg/kg, 75 mg/kg, 80 mg/kg, 85 mg/kg, 90 mg/kg, 95 mg/kg, 100 mg/kg, 105 mg/kg, 110 mg/kg, 115 mg/kg, 120 mg/kg, 125 mg/kg, 130 mg/kg, 135 mg/kg, 140 mg/kg, 145 mg/kg, 150 mg/kg, 155 mg/kg, 160 mg/kg, 165 mg/kg, 170 mg/kg, 175 mg/kg, 180 mg/kg, 185 mg/kg, 190 mg/kg, 195 mg/kg, 200 mg/kg, and any fraction or integer in between any two of the preceding amounts.

In some embodiments, an nNOS inhibitor composition comprising one or more shRNAs, may be administered as a suitable dosage form, as described herein, to treat, ameliorate, and/or reduce the severity of NSC-lung cancer or one or more symptoms thereof in an NSC-lung cancer subject. In certain embodiments, an nNOS inhibitor composition comprising one or more shRNAs selected from the group consisting of SEQ ID NO. 9, SEQ ID NO. 10, SEQ ID NO. 11, SEQ ID NO. 12, SEQ ID NO. 13, SEQ ID NO. 14, and any combination thereof, may be administered as a suitable dosage form, as described herein, to treat, ameliorate, and/or reduce the severity of NSC-lung cancer or one or more symptoms thereof in an NSC-lung cancer subject. In some embodiments, an nNOS inhibitor composition comprising one or more shRNAs selected from the group consisting of SEQ ID NO. 9, SEQ ID NO. 10, SEQ ID NO. 11, SEQ ID NO. 12, SEQ ID NO. 13, SEQ ID NO. 14, and any combination thereof, may be administered to an NSC-lung cancer subject to treat, ameliorate, and/or reduce the severity of NSC-lung cancer or one or more symptoms thereof in the NSC-lung cancer subject: (i) via a route selected from the group consisting of buccally, inhalation, intraarterially, intraarticularly, intracardially, intracerebroventricularly, intradermally, intramedullary, intramuscularly, intraperitoneally, intrathecally, intravenously, intraventricularly, nasally, ophthalmically, orally, rectally, sublabially, sublingually, subcutaneously, topically, and transdermally; and/or (ii) with a frequency selected from the group consisting of four times daily, three times daily, twice daily, once daily, once every two days, once every three days, once every four days, once every five days, once every six days, once a week, once every eight days, once every nine days, once every ten days, once every two weeks, once every three weeks, and once a month; and/or (iii) at a dosage (based on subject weight) selected from the group consisting of 0.1 μg/kg, 0.5 μg/kg, 1 μg/kg, 1.5 μg/kg, 2.0 μg/kg, 2.5 μg/kg, 3.0 μg/kg, 3.5 μg/kg, 4.0 μg/kg, 4.5 μg/kg, 5.0 μg/kg, 10 μg/kg, 15 μg/kg, 20 μg/kg, 25 μg/kg, 30 μg/kg, 35 μg/kg, 40 μg/kg, 45 μg/kg, 50 μg/kg, 55 μg/kg, 60 μg/kg, 65 μg/kg, 70 μg/kg, 75 μg/kg, 80 μg/kg, 85 μg/kg, 90 μg/kg, 95 μg/kg, 100 μg/kg, 150 μg/kg, 200 μg/kg, 250 μg/kg, 300 μg/kg, 350 μg/kg, 400 μg/kg, 450 μg/kg, 500 μg/kg, 550 μg/kg, 600 μg/kg, 650 μg/kg, 700 μg/kg, 750 μg/kg, 800 μg/kg, 850 μg/kg, 900 μg/kg, 1 mg/kg, 1.5 mg/kg, 2.0 mg/kg, 2.5 mg/kg, 3 mg/kg, 3.5 mg/kg, 4.0 mg/kg, 4.5 mg/kg, 5 mg/kg, 5.5 mg/kg, 6 mg/kg, 6.5 mg/kg, 7 mg/kg, 7.5 mg/kg, 8 mg/kg, 8.5 mg/kg, 9 mg/kg, 9.5 mg/kg, 10 mg/kg 10.5 mg/kg, 11 mg/kg, 11.5 mg/kg, 12 mg/kg, 12.5 mg/kg, 13 mg/kg, 13.5 mg/kg, 14 mg/kg, 14.5 mg/kg, 15 mg/kg, 16 mg/kg, 17 mg/kg, 18 mg/kg, 19 mg/kg, 20 mg/kg, 21 mg/kg, 22 mg/kg, 23 mg/kg, 24 mg/kg, 25 mg/kg, 30 mg/kg, 35 mg/kg, 40 mg/kg, 45 mg/kg, 50 mg/kg, 55 mg/kg, 60 mg/kg, 65 mg/kg, 70 mg/kg, 75 mg/kg, 80 mg/kg, 85 mg/kg, 90 mg/kg, 95 mg/kg, 100 mg/kg, 105 mg/kg, 110 mg/kg, 115 mg/kg, 120 mg/kg, 125 mg/kg, 130 mg/kg, 135 mg/kg, 140 mg/kg, 145 mg/kg, 150 mg/kg, 155 mg/kg, 160 mg/kg, 165 mg/kg, 170 mg/kg, 175 mg/kg, 180 mg/kg, 185 mg/kg, 190 mg/kg, 195 mg/kg, 200 mg/kg, and any fraction or integer in between any two of the preceding amounts.

In some embodiments, an nNOS inhibitor composition comprising one or more miRNAs, may be administered as a suitable dosage form, as described herein, to treat, ameliorate, and/or reduce the severity of NSC-lung cancer or one or more symptoms thereof in an NSC-lung cancer subject. In certain embodiments, an nNOS inhibitor composition comprising one or more miRNAs selected from the group consisting of miR-146a, hsa-miR-146a-5p, hsa-miR-1273f, hsa-miR-143-3p, hsa-miR-147a, hsa-miR-3622a-5p, hsa-miR-3911, hsa-miR-4269, hsa-miR-4708-5p, hsa-miR-4710, hsa-miR-4770, hsa-miR-4792, hsa-miR-574-5p, hsa-miR-6088, hsa-miR-644a, hsa-miR-6715b-5p, hsa-miR-6768-5p, hsa-miR-6867-5p, hsa-miR-7855-5p, hsa-miR-8485, and any combination thereof, may be administered as a suitable dosage form, as described herein, to treat, ameliorate, and/or reduce the severity of NSC-lung cancer or one or more symptoms thereof in an NSC-lung cancer subject. In some embodiments, an nNOS inhibitor composition comprising one or more miRNAs selected from the group consisting of miR-146a, hsa-miR-146a-5p, hsa-miR-1273f, hsa-miR-143-3p, hsa-miR-147a, hsa-miR-3622a-5p, hsa-miR-3911, hsa-miR-4269, hsa-miR-4708-5p, hsa-miR-4710, hsa-miR-4770, hsa-miR-4792, hsa-miR-574-5p, hsa-miR-6088, hsa-miR-644a, hsa-miR-6715b-5p, hsa-miR-6768-5p, hsa-miR-6867-5p, hsa-miR-7855-5p, hsa-miR-8485, and any combination thereof, may be administered to an NSC-lung cancer subject to treat, ameliorate, and/or reduce the severity of NSC-lung cancer or one or more symptoms thereof in the NSC-lung cancer subject: (i) via a route selected from the group consisting of buccally, inhalation, intraarterially, intraarticularly, intracardially, intracerebroventricularly, intradermally, intramedullary, intramuscularly, intraperitoneally, intrathecally, intravenously, intraventricularly, nasally, ophthalmically, orally, rectally, sublabially, sublingually, subcutaneously, topically, and transdermally; and/or (ii) with a frequency selected from the group consisting of four times daily, three times daily, twice daily, once daily, once every two days, once every three days, once every four days, once every five days, once every six days, once a week, once every eight days, once every nine days, once every ten days, once every two weeks, once every three weeks, and once a month; and/or (iii) at a dosage (based on subject weight) selected from the group consisting of 0.1 μg/kg, 0.5 μg/kg, 1 μg/kg, 1.5 μg/kg, 2.0 μg/kg, 2.5 μg/kg, 3.0 μg/kg, 3.5 μg/kg, 4.0 μg/kg, 4.5 μg/kg, 5.0 μg/kg, 10 μg/kg, 15 μg/kg, 20 μg/kg, 25 μg/kg, 30 μg/kg, 35 μg/kg, 40 μg/kg, 45 μg/kg, 50 μg/kg, 55 μg/kg, 60 μg/kg, 65 μg/kg, 70 μg/kg, 75 μg/kg, 80 μg/kg, 85 μg/kg, 90 μg/kg, 95 μg/kg, 100 μg/kg, 150 μg/kg, 200 μg/kg, 250 μg/kg, 300 μg/kg, 350 μg/kg, 400 μg/kg, 450 μg/kg, 500 μg/kg, 550 μg/kg, 600 μg/kg, 650 μg/kg, 700 μg/kg, 750 μg/kg, 800 μg/kg, 850 μg/kg, 900 μg/kg, 1 mg/kg, 1.5 mg/kg, 2.0 mg/kg, 2.5 mg/kg, 3 mg/kg, 3.5 mg/kg, 4.0 mg/kg, 4.5 mg/kg, 5 mg/kg, 5.5 mg/kg, 6 mg/kg, 6.5 mg/kg, 7 mg/kg, 7.5 mg/kg, 8 mg/kg, 8.5 mg/kg, 9 mg/kg, 9.5 mg/kg, 10 mg/kg 10.5 mg/kg, 11 mg/kg, 11.5 mg/kg, 12 mg/kg, 12.5 mg/kg, 13 mg/kg, 13.5 mg/kg, 14 mg/kg, 14.5 mg/kg, 15 mg/kg, 16 mg/kg, 17 mg/kg, 18 mg/kg, 19 mg/kg, 20 mg/kg, 21 mg/kg, 22 mg/kg, 23 mg/kg, 24 mg/kg, 25 mg/kg, 30 mg/kg, 35 mg/kg, 40 mg/kg, 45 mg/kg, 50 mg/kg, 55 mg/kg, 60 mg/kg, 65 mg/kg, 70 mg/kg, 75 mg/kg, 80 mg/kg, 85 mg/kg, 90 mg/kg, 95 mg/kg, 100 mg/kg, 105 mg/kg, 110 mg/kg, 115 mg/kg, 120 mg/kg, 125 mg/kg, 130 mg/kg, 135 mg/kg, 140 mg/kg, 145 mg/kg, 150 mg/kg, 155 mg/kg, 160 mg/kg, 165 mg/kg, 170 mg/kg, 175 mg/kg, 180 mg/kg, 185 mg/kg, 190 mg/kg, 195 mg/kg, 200 mg/kg, and any fraction or integer in between any two of the preceding amounts.

In some embodiments, an nNOS inhibitor composition comprising one or more CRISPR/Cas systems, may be administered as a suitable dosage form, as described herein, to treat, ameliorate, and/or reduce the severity of NSC-lung cancer or one or more symptoms thereof in an NSC-lung cancer subject. In certain embodiments, an nNOS inhibitor composition comprising one or more CRISPR/Cas systems comprising an endonuclease selected from the group consisting of Cas1, Cas2, Cas3, Cas4, Cas5, Cas6, Cas7, Cas8a, Cas8b, Cas8c, Cas9, Cas10, Cas10d, Cas12, Cas12a, Cas12b, Cas12c, Cas12d, Cas12e, Cas12f, Cas12g, Cas12h, Cas12i, Cas12k, C2c4, C2c8, C2c9, Cas13, Cas13a, Cas13b, Cas13c, Cas13d, Cas13x.1, Cse1, Cse2, Csy1, Csy2, Csy3, GSU0054, Csm2, Cmr5, Csx10, Csx11, Csf1, and Csn2, may be administered as a suitable dosage form, as described herein, to treat, ameliorate, and/or reduce the severity of NSC-lung cancer or one or more symptoms thereof in an NSC-lung cancer subject. In some embodiments, an nNOS inhibitor composition comprising one or more CRISPR/Cas systems comprising an endonuclease selected from the group consisting of Cas1, Cas2, Cas3, Cas4, Cas5, Cas6, Cas7, Cas8a, Cas8b, Cas8c, Cas9, Cas10, Cas10d, Cas12, Cas12a, Cas12b, Cas12c, Cas12d, Cas12e, Cas12f, Cas12g, Cas12h, Cas12i, Cas12k, C2c4, C2c8, C2c9, Cas13, Cas13a, Cas13b, Cas13c, Cas13d, Cas13x.1, Cse1, Cse2, Csy1, Csy2, Csy3, GSU0054, Csm2, Cmr5, Csx10, Csx11, Csf1, and Csn2, may be administered to an NSC-lung cancer subject to treat, ameliorate, and/or reduce the severity of NSC-lung cancer or one or more symptoms thereof in the NSC-lung cancer subject: (i) via a route selected from the group consisting of buccally, inhalation, intraarterially, intraarticularly, intracardially, intracerebroventricularly, intradermally, intramedullary, intramuscularly, intraperitoneally, intrathecally, intravenously, intraventricularly, nasally, ophthalmically, orally, rectally, sublabially, sublingually, subcutaneously, topically, and transdermally; and/or (ii) with a frequency selected from the group consisting of four times daily, three times daily, twice daily, once daily, once every two days, once every three days, once every four days, once every five days, once every six days, once a week, once every eight days, once every nine days, once every ten days, once every two weeks, once every three weeks, and once a month; and/or (iii) at a dosage (based on subject weight) selected from the group consisting of 0.1 μg/kg, 0.5 μg/kg, 1 μg/kg, 1.5 μg/kg, 2.0 μg/kg, 2.5 μg/kg, 3.0 μg/kg, 3.5 μg/kg, 4.0 μg/kg, 4.5 μg/kg, 5.0 μg/kg, 10 μg/kg, 15 μg/kg, 20 μg/kg, 25 μg/kg, 30 μg/kg, 35 μg/kg, 40 μg/kg, 45 μg/kg, 50 μg/kg, 55 μg/kg, 60 μg/kg, 65 μg/kg, 70 μg/kg, 75 μg/kg, 80 μg/kg, 85 μg/kg, 90 μg/kg, 95 μg/kg, 100 μg/kg, 150 μg/kg, 200 μg/kg, 250 μg/kg, 300 μg/kg, 350 μg/kg, 400 μg/kg, 450 μg/kg, 500 μg/kg, 550 μg/kg, 600 μg/kg, 650 μg/kg, 700 μg/kg, 750 μg/kg, 800 μg/kg, 850 μg/kg, 900 μg/kg, 1 mg/kg, 1.5 mg/kg, 2.0 mg/kg, 2.5 mg/kg, 3 mg/kg, 3.5 mg/kg, 4.0 mg/kg, 4.5 mg/kg, 5 mg/kg, 5.5 mg/kg, 6 mg/kg, 6.5 mg/kg, 7 mg/kg, 7.5 mg/kg, 8 mg/kg, 8.5 mg/kg, 9 mg/kg, 9.5 mg/kg, 10 mg/kg 10.5 mg/kg, 11 mg/kg, 11.5 mg/kg, 12 mg/kg, 12.5 mg/kg, 13 mg/kg, 13.5 mg/kg, 14 mg/kg, 14.5 mg/kg, 15 mg/kg, 16 mg/kg, 17 mg/kg, 18 mg/kg, 19 mg/kg, 20 mg/kg, 21 mg/kg, 22 mg/kg, 23 mg/kg, 24 mg/kg, 25 mg/kg, 30 mg/kg, 35 mg/kg, 40 mg/kg, 45 mg/kg, 50 mg/kg, 55 mg/kg, 60 mg/kg, 65 mg/kg, 70 mg/kg, 75 mg/kg, 80 mg/kg, 85 mg/kg, 90 mg/kg, 95 mg/kg, 100 mg/kg, 105 mg/kg, 110 mg/kg, 115 mg/kg, 120 mg/kg, 125 mg/kg, 130 mg/kg, 135 mg/kg, 140 mg/kg, 145 mg/kg, 150 mg/kg, 155 mg/kg, 160 mg/kg, 165 mg/kg, 170 mg/kg, 175 mg/kg, 180 mg/kg, 185 mg/kg, 190 mg/kg, 195 mg/kg, 200 mg/kg, and any fraction or integer in between any two of the preceding amounts.

In some embodiments, an nNOS inhibitor composition comprising one or more antibodies, may be administered as a suitable dosage form, as described herein, to treat, ameliorate, and/or reduce the severity of NSC-lung cancer or one or more symptoms thereof in an NSC-lung cancer subject. In certain embodiments, an nNOS inhibitor composition comprising one or more antibodies selected from the group consisting of 37-2800 (from ThermoFisher), EP1855Y (from abeam), EPR 24351-6 (from abeam), and any combination thereof, may be administered as a suitable dosage form, as described herein, to treat, ameliorate, and/or reduce the severity of NSC-lung cancer or one or more symptoms thereof in an NSC-lung cancer subject. In some embodiments, an nNOS inhibitor composition comprising one or more antibodies selected from the group consisting of 37-2800 (from ThermoFisher), EP1855Y (from abeam), EPR 24351-6 (from abcam), and any combination thereof, may be administered to an NSC-lung cancer subject to treat, ameliorate, and/or reduce the severity of NSC-lung cancer or one or more symptoms thereof in the NSC-lung cancer subject: (i) via a route selected from the group consisting of buccally, inhalation, intraarterially, intraarticularly, intracardially, intracerebroventricularly, intradermally, intramedullary, intramuscularly, intraperitoneally, intrathecally, intravenously, intraventricularly, nasally, ophthalmically, orally, rectally, sublabially, sublingually, subcutaneously, topically, and transdermally; and/or (ii) with a frequency selected from the group consisting of four times daily, three times daily, twice daily, once daily, once every two days, once every three days, once every four days, once every five days, once every six days, once a week, once every eight days, once every nine days, once every ten days, once every two weeks, once every three weeks, and once a month; and/or (iii) at a dosage (based on subject weight) selected from the group consisting of 0.1 μg/kg, 0.5 μg/kg, 1 μg/kg, 1.5 μg/kg, 2.0 μg/kg, 2.5 μg/kg, 3.0 μg/kg, 3.5 μg/kg, 4.0 μg/kg, 4.5 μg/kg, 5.0 μg/kg, 10 μg/kg, 15 μg/kg, 20 μg/kg, 25 μg/kg, 30 μg/kg, 35 μg/kg, 40 μg/kg, 45 μg/kg, 50 μg/kg, 55 μg/kg, 60 μg/kg, 65 μg/kg, 70 μg/kg, 75 μg/kg, 80 μg/kg, 85 μg/kg, 90 μg/kg, 95 μg/kg, 100 μg/kg, 150 μg/kg, 200 μg/kg, 250 μg/kg, 300 μg/kg, 350 μg/kg, 400 μg/kg, 450 μg/kg, 500 μg/kg, 550 μg/kg, 600 μg/kg, 650 μg/kg, 700 μg/kg, 750 μg/kg, 800 μg/kg, 850 μg/kg, 900 μg/kg, 1 mg/kg, 1.5 mg/kg, 2.0 mg/kg, 2.5 mg/kg, 3 mg/kg, 3.5 mg/kg, 4.0 mg/kg, 4.5 mg/kg, 5 mg/kg, 5.5 mg/kg, 6 mg/kg, 6.5 mg/kg, 7 mg/kg, 7.5 mg/kg, 8 mg/kg, 8.5 mg/kg, 9 mg/kg, 9.5 mg/kg, 10 mg/kg 10.5 mg/kg, 11 mg/kg, 11.5 mg/kg, 12 mg/kg, 12.5 mg/kg, 13 mg/kg, 13.5 mg/kg, 14 mg/kg, 14.5 mg/kg, 15 mg/kg, 16 mg/kg, 17 mg/kg, 18 mg/kg, 19 mg/kg, 20 mg/kg, 21 mg/kg, 22 mg/kg, 23 mg/kg, 24 mg/kg, 25 mg/kg, 30 mg/kg, 35 mg/kg, 40 mg/kg, 45 mg/kg, 50 mg/kg, 55 mg/kg, 60 mg/kg, 65 mg/kg, 70 mg/kg, 75 mg/kg, 80 mg/kg, 85 mg/kg, 90 mg/kg, 95 mg/kg, 100 mg/kg, 105 mg/kg, 110 mg/kg, 115 mg/kg, 120 mg/kg, 125 mg/kg, 130 mg/kg, 135 mg/kg, 140 mg/kg, 145 mg/kg, 150 mg/kg, 155 mg/kg, 160 mg/kg, 165 mg/kg, 170 mg/kg, 175 mg/kg, 180 mg/kg, 185 mg/kg, 190 mg/kg, 195 mg/kg, 200 mg/kg, and any fraction or integer in between any two of the preceding amounts.

In some embodiments, an nNOS inhibitor composition comprising one or more amino acid-based compounds, may be administered as a suitable dosage form, as described herein, to treat, ameliorate, and/or reduce the severity of NSC-lung cancer or one or more symptoms thereof in an NSC-lung cancer subject. In certain embodiments, an nNOS inhibitor composition comprising one or more amino acid-compounds that bind an active site of nNOS selected from the group consisting of Phenylalanine 589, Tryptophan 592, Tyrosine 593, Glutamate 597, Aspartate 602, Arginine 601, Methionine 341, Histidine 342, and Tyrosine711, may be administered as a suitable dosage form, as described herein, to treat, ameliorate, and/or reduce the severity of NSC-lung cancer or one or more symptoms thereof in an NSC-lung cancer subject. In some embodiments, an nNOS inhibitor composition comprising one or more amino acid-compounds that bind an active site of nNOS selected from the group consisting of Phenylalanine 589, Tryptophan 592, Tyrosine 593, Glutamate 597, Aspartate 602, Arginine 601, Methionine 341, Histidine 342, and Tyrosine711, may be administered to an NSC-lung cancer subject to treat, ameliorate, and/or reduce the severity of NSC-lung cancer or one or more symptoms thereof in the NSC-lung cancer subject: (i) via a route selected from the group consisting of buccally, inhalation, intraarterially, intraarticularly, intracardially, intracerebroventricularly, intradermally, intramedullary, intramuscularly, intraperitoneally, intrathecally, intravenously, intraventricularly, nasally, ophthalmically, orally, rectally, sublabially, sublingually, subcutaneously, topically, and transdermally; and/or (ii) with a frequency selected from the group consisting of four times daily, three times daily, twice daily, once daily, once every two days, once every three days, once every four days, once every five days, once every six days, once a week, once every eight days, once every nine days, once every ten days, once every two weeks, once every three weeks, and once a month; and/or (iii) at a dosage (based on subject weight) selected from the group consisting of 0.1 μg/kg, 0.5 μg/kg, 1 μg/kg, 1.5 μg/kg, 2.0 μg/kg, 2.5 μg/kg, 3.0 μg/kg, 3.5 μg/kg, 4.0 μg/kg, 4.5 μg/kg, 5.0 μg/kg, 10 μg/kg, 15 μg/kg, 20 μg/kg, 25 μg/kg, 30 μg/kg, 35 μg/kg, 40 μg/kg, 45 μg/kg, 50 μg/kg, 55 μg/kg, 60 μg/kg, 65 μg/kg, 70 μg/kg, 75 μg/kg, 80 μg/kg, 85 μg/kg, 90 μg/kg, 95 μg/kg, 100 μg/kg, 150 μg/kg, 200 μg/kg, 250 μg/kg, 300 μg/kg, 350 μg/kg, 400 μg/kg, 450 μg/kg, 500 μg/kg, 550 μg/kg, 600 μg/kg, 650 μg/kg, 700 μg/kg, 750 μg/kg, 800 μg/kg, 850 μg/kg, 900 μg/kg, 1 mg/kg, 1.5 mg/kg, 2.0 mg/kg, 2.5 mg/kg, 3 mg/kg, 3.5 mg/kg, 4.0 mg/kg, 4.5 mg/kg, 5 mg/kg, 5.5 mg/kg, 6 mg/kg, 6.5 mg/kg, 7 mg/kg, 7.5 mg/kg, 8 mg/kg, 8.5 mg/kg, 9 mg/kg, 9.5 mg/kg, 10 mg/kg 10.5 mg/kg, 11 mg/kg, 11.5 mg/kg, 12 mg/kg, 12.5 mg/kg, 13 mg/kg, 13.5 mg/kg, 14 mg/kg, 14.5 mg/kg, 15 mg/kg, 16 mg/kg, 17 mg/kg, 18 mg/kg, 19 mg/kg, 20 mg/kg, 21 mg/kg, 22 mg/kg, 23 mg/kg, 24 mg/kg, 25 mg/kg, 30 mg/kg, 35 mg/kg, 40 mg/kg, 45 mg/kg, 50 mg/kg, 55 mg/kg, 60 mg/kg, 65 mg/kg, 70 mg/kg, 75 mg/kg, 80 mg/kg, 85 mg/kg, 90 mg/kg, 95 mg/kg, 100 mg/kg, 105 mg/kg, 110 mg/kg, 115 mg/kg, 120 mg/kg, 125 mg/kg, 130 mg/kg, 135 mg/kg, 140 mg/kg, 145 mg/kg, 150 mg/kg, 155 mg/kg, 160 mg/kg, 165 mg/kg, 170 mg/kg, 175 mg/kg, 180 mg/kg, 185 mg/kg, 190 mg/kg, 195 mg/kg, 200 mg/kg, and any fraction or integer in between any two of the preceding amounts.

In some embodiments, an NSC-lung cancer subject, as described herein, may be a female child or a male child. In certain embodiments, an NSC-lung cancer subject, as described herein, may be a female adult or a male adult.

In some embodiments, an NSC-lung cancer a subject, as described herein, may have an abnormal expression of one or more antigens compared to an equivalent subject that does not have an NSC-lung cancer. As used herein, the term “equivalent subject,” refers to a subject of the same sex (male/female) and age range (child/adult) of the NSC-lung cancer subject. For purposes of disclosure, the term “antigen,” when used with respect to abnormal expression, may define one or more cluster of differentiation (“CD”) antigens, such as, for example, CD15, CD44, CD70, CD133, as well as any CD antigen that has an abnormal expression in an NSC-lung cancer that would be readily envisaged by one skilled in the art. In certain embodiments, abnormal expression of an antigen may define an abnormal expression of CD15, an abnormal expression of CD44, an abnormal expression of CD70, an abnormal expression of CD133, an abnormal expression of a CD antigen that has an abnormal expression in an NSC-lung cancer that would be readily envisaged by one skilled in the art, and any combination thereof. In some embodiments, an “abnormal expression,” can refer to a reduced expression of an antigen, as described herein. In certain embodiments, an “abnormal expression,” can refer to an overexpression of an antigen, as described herein. Whether “abnormal expression,” refers to a reduced expression of an antigen or an overexpression of an antigen would be immediately envisaged by the skilled artisan when viewing the term in the context of the particular antigen and NSC-lung cancer disclosed. For example, in the context of CD133 and non-small cell carcinomas (NSC-lung cancers), one skilled in the art would immediately envisage that “abnormal expression” refers to an overexpression of CD133. Treatment, amelioration, and/or reduction in the severity of an NSC-lung cancer or one or more symptoms thereof may be characterized by a restoration to a normal level of expression of one or more antigens from their abnormal state, as described herein. For example, in the context of CD133 and non-small cell carcinomas, treatment, amelioration, and/or reduction in the severity of an NSC-lung cancer or one or more symptoms thereof may be characterized by a reduced expression of CD133.

In certain embodiments, an NSC-lung cancer subject, as described herein, may have an abnormal expression of one or more proteins compared to an equivalent subject that does not have an NSC-lung cancer. For purposes of disclosure, the term “protein,” when used with respect to abnormal expression, may define one or more proteins and/or enzymes, such as, for example, ALDH1A3, mTOR, p-mTOR, TSC-2, AKT, pAKT, RPS6, p-RPS6, Syp, VEGF, PIGF, as well as any protein or enzyme that has an abnormal expression in a NSC-lung cancer that would be readily envisaged by one skilled in the art. In certain embodiments, abnormal expression of a protein may define an abnormal expression of ALDH1A3, an abnormal expression of mTOR, an abnormal expression of p-mTOR, an abnormal expression of TSC-2, an abnormal expression of AKT, an abnormal expression of pAKT, an abnormal expression of RPS6, an abnormal expression of p-RPS6, an abnormal expression of Syp, an abnormal expression of VEGF, an abnormal expression of PIGF, an abnormal expression of a protein or enzyme that has an abnormal expression in a NSC-lung cancer that would be readily envisaged by one skilled in the art, and any combination thereof. In some embodiments, an “abnormal expression,” can refer to a reduced expression of a protein, as described herein. In certain embodiments, an “abnormal expression,” can refer to an overexpression of a protein, as described herein. Whether “abnormal expression,” refers to a reduced expression of a protein or an overexpression of a protein would be immediately envisaged by the skilled artisan when viewing the term in the context of the particular protein and NSC-lung cancer disclosed. For example, in the context of non-small cell lung carcinoma (an NSC-lung cancer), one skilled in the art would immediately envisage that “abnormal expression” refers to an overexpression of mTOR. Treatment, amelioration, and/or reduction in the severity of an NSC-lung cancer or one or more symptoms thereof may be characterized by a restoration to a normal level of expression of one or more proteins from their abnormal state, as described herein. For example, in the context of non-small cell lung carcinoma, treatment, amelioration, and/or reduction in the severity of an NSC-lung cancer or one or more symptoms thereof may be characterized by a reduced expression of mTOR.

In certain embodiments, an NSC-lung cancer subject, as described herein, may have an increased expression of one or more NOS isoforms compared to an equivalent subject that does not have an NSC-lung cancer. For purposes of disclosure, the term “NOS isoform,” refers to nNOS, iNOS, and/or eNOS. In certain embodiments, an NSC-lung cancer subject, as described herein, may have an increased expression of nNOS compared to an equivalent subject that does not have an NSC-lung cancer. In some embodiments, an NSC-lung cancer subject, as described herein, may have an increased expression of iNOS compared to an equivalent subject that does not have an NSC-lung cancer. In certain embodiments, an NSC-lung cancer subject, as described herein, may have an increased expression of eNOS compared to an equivalent subject that does not have an NSC-lung cancer. In some embodiments, an NSC-lung cancer subject, as described herein, may have an increased expression of nNOS and iNOS compared to an equivalent subject that does not have an NSC-lung cancer. In certain embodiments, an NSC-lung cancer subject, as described herein, may have an increased expression of nNOS and eNOS compared to an equivalent subject that does not have an NSC-lung cancer. In some embodiments, an NSC-lung cancer subject, as described herein, may have an increased expression of iNOS and eNOS compared to an equivalent subject that does not have an NSC-lung cancer. In certain embodiments, an NSC-lung cancer subject, as described herein, may have an increased expression of nNOS, iNOS, and eNOS compared to an equivalent subject that does not have an NSC-lung cancer. Whether a “NOS isoform,” has an increased expression would be immediately envisaged by the skilled artisan when viewing the term in the context of the particular NOS isoform and NSC-lung cancer disclosed. For example, in the context of non-small cell lung carcinoma (an NSC-lung cancer), one skilled in the art would immediately envisage that iNOS has an increased expression. Treatment, amelioration, and/or reduction in the severity of an NSC-lung cancer or one or more symptoms thereof may be characterized by a reduced expression of one or more NOS isoforms from their increased state, as described herein. For example, in the context of non-small cell lung carcinoma, treatment, amelioration, and/or reduction in the severity of an NSC-lung cancer or one or more symptoms thereof may be characterized by a reduced expression of iNOS. For purposes of disclosure, reduction/inhibition of the expression of one or more NOS isoforms may be characterized by a reduction in expression of one or more NOS isoforms by about at least 10%, about at least 20%, about at least 30%, or about at least 40%. In some embodiments, reduction/inhibition of the expression of one or more NOS isoforms may further be characterized by a lack reduction of other, non-implicated NOS isoforms, such as, for example, no more than about 10%, no more than about 5%, no more than about 2%, or no more than about 1% reduction/inhibition of the expression of non-implicated NOS isoforms. As used herein, the term “non-implicated NOS isoforms,” refers to NOS isoforms that do not have an increased expression in a particular NSC-lung cancer. By way of example, in the context of non-small cell lung carcinoma, if iNOS has an increased expression, nNOS and/or eNOS would define a “non-implicated” NOS isoform. Whether a NOS isoform is a “non-implicated NOS isoform,” would be immediately envisaged by the skilled artisan when viewing the term in the context of the particular NOS isoform disclosed, as well as the NSC-lung cancer.

In some embodiments, an NSC-lung cancer subject, as described herein, may have an elevated level of one or more NO-indicators compared to an equivalent subject that does not have an NSC-lung cancer. As used herein, the term “NO-indicator,” can refer to 3-nitrotyrosine (3-Ntyr), NADPH-diaphorase, total nitrite concentration, total nitrate concentration, total nitrite and nitrate concentration, as well as any NO-indicator that has an elevated level in an NSC-lung cancer that would be readily envisaged by one skilled in the art. For example, in the context of non-small cell carcinoma (an NSC-lung cancer), one skilled in the art would immediately envisage that 3-Ntyr can be present at an elevated level. Treatment, amelioration, and/or reduction in the severity of an NSC-lung cancer or one or more symptoms thereof may be characterized by a reduction in the level of one or more NO-indicators. For example, in the context of small cell carcinoma, treatment, amelioration, and/or reduction in the severity of an NSC-lung cancer or one or more symptoms thereof may be characterized by a reduction in the level of 3-Ntyr.

In certain embodiments, a NOS inhibitor composition, as described herein, may be administered as a suitable dosage form, as described herein, to treat, ameliorate, and/or reduce the severity of an NSC-lung cancer or one or more symptoms thereof, as an alternative to a conventional therapy for treating, ameliorating, and/or reducing the severity of an NSC-lung cancer or one or more symptoms thereof. For purposes of disclosure, a “conventional therapy,” as described herein, may refer to a treatment that would be recommended by one skilled in the art to treat a NSC-lung cancer, and may define one or more of radiation, chemotherapy, immunotherapy, cell-based therapy, gene therapy, and the like—one skilled in the art would immediately envisage a “conventional therapy” based on the particular NSC-lung cancer disclosed, as well as characteristics of a NSC-lung cancer subject, such as, for example, the subject's sex, age, stage of the NSC-lung cancer, genotype of the NSC-lung cancer, and the like. Treatment, amelioration, and/or reduction in the severity of an NSC-lung cancer or one or more symptoms thereof may be characterized by an improved outcome compared to a conventional therapy, as described herein. For purposes of the disclosure, an “improved outcome” may define any one of the following: (i) a larger reduction in tumor volume and/or mass compared to a subject administered an equivalent dose of a conventional therapy; (ii) an improvement in the expected rate of survival of a subject, at, for example, 1 month, 3 months, 6 months, 9 months, 1 year, 2 years, 3 years, 5 years, 10 years, or more, compared to the expected rate of survival of a subject administered an equivalent dose of a conventional therapy; (iii) a reduction in the prevalence of side effects resulting from the NOS inhibitor composition compared to a subject administered an equivalent dose of a conventional therapy; (iv) a reduction in the severity of side effects resulting from the NOS inhibitor composition compared to a subject administered an equivalent dose of a conventional therapy; (v) a reduction in the dosage required to achieve an equivalent reduction in tumor volume compared to a subject administered a conventional therapy; (vi) a reduction in the dosage required to achieve an equivalent expected rate of survival of a subject compared to a subject administered a conventional therapy; (vii) a reduction in the likelihood of a recurrence of the NSC-lung cancer occurring in a subject compared to a subject administered an equivalent dose of a conventional therapy; (viii) a reduction in the likelihood of a metastasis occurring in a subject compared to a subject administered an equivalent dose of a conventional therapy; (ix) a reduction in the time required to be at a medical facility for treatment compared to a subject administered an equivalent dose of a conventional therapy; (x) a reduction in the cost associated with treatment compared to a subject administered an equivalent dose of a conventional therapy; as well as any other improved outcome not expressly recited that would be readily envisaged by one skilled in the art.

In some embodiments, a NOS inhibitor composition, as described herein, may be administered as a suitable dosage form, as described herein, to treat, ameliorate, and/or reduce the severity of an NSC-lung cancer or one or more symptoms thereof, wherein the NOS inhibitor composition comprises a first chemical NOS inhibitor and a second chemical NOS inhibitor. In certain embodiments, the first chemical NOS inhibitor may be administered concurrently with the second chemical NOS inhibitor. In some embodiments, the first chemical NOS inhibitor may be administered prior to the second chemical NOS inhibitor. In certain embodiments, the first chemical NOS inhibitor may be administered subsequently to the second chemical NOS inhibitor. Treatment, amelioration, and/or reduction in the severity of an NSC-lung cancer or one or more symptoms thereof may be characterized by an improved outcome compared an alternative treatment. As used herein, the term “alternative treatment,” may define one or more of a conventional therapy, as described herein, a NOS inhibitor composition comprising a single chemical NOS inhibitor, as described herein, and a NOS inhibitor composition comprising a single biological NOS inhibitor, as described herein. For purposes of the disclosure, an “improved outcome” may define any one of the following: (i) a larger reduction in tumor volume and/or mass compared an alternative treatment; (ii) an improvement in the expected rate of survival of a subject, at, for example, 1 month, 3 months, 6 months, 9 months, 1 year, 2 years, 3 years, 5 years, 10 years, or more, compared to the expected rate of survival of a subject administered an alternative treatment; (iii) a reduction in the prevalence of side effects resulting from the NOS inhibitor composition comprising at least two chemical NOS inhibitors compared to a subject administered an alternative treatment; (iv) a reduction in the severity of side effects resulting from the NOS inhibitor composition comprising at least two chemical NOS inhibitors compared to a subject administered an alternative treatment; (v) a reduction in the dosage required to achieve an equivalent reduction in tumor volume compared to a subject administered an alternative treatment; (vi) a reduction in the dosage required to achieve an equivalent expected rate of survival of a subject compared to a subject administered an alternative treatment; (vii) a reduction in the likelihood of a recurrence of the NSC-lung cancer in a subject compared to a subject administered an alternative treatment; (viii) a reduction in the likelihood of a metastasis occurring in a subject compared to a subject administered an alternative treatment; (ix) a reduction in the time required to be at a medical facility for treatment compared to a subject administered an alternative treatment; (x) a reduction in the cost associated with treatment compared to a subject administered an alternative treatment; as well as any other improved outcome not expressly recited that would be readily envisaged by one skilled in the art.

In certain embodiments, a NOS inhibitor composition, as described herein, may be used to treat, ameliorate, and/or reduce the severity of an NSC-lung cancer or one or more symptoms thereof, wherein the NOS inhibitor composition comprises at least one chemical NOS inhibitor and at least one biological NOS inhibitor. In some embodiments, the at least one chemical NOS inhibitor may be administered concurrently with the at least one biological NOS inhibitor. In certain embodiments, the at least one chemical NOS inhibitor may be administered prior to the at least one biological NOS inhibitor. In some embodiments, the at least one chemical NOS inhibitor may be administered subsequently to the at least one biological NOS inhibitor. Treatment, amelioration, and/or reduction in the severity of an NSC-lung cancer or one or more symptoms thereof may be characterized by an improved outcome compared an alternative treatment. As used herein, the term “alternative treatment,” may define one or more of a conventional therapy, as described herein, a NOS inhibitor composition comprising a single chemical NOS inhibitor, as described herein, and a NOS inhibitor composition comprising a single biological NOS inhibitor, as described herein. For purposes of the disclosure, an “improved outcome” may define any one of the following: (i) a larger reduction in tumor volume and/or mass compared an alternative treatment; (ii) an improvement in the expected rate of survival of a subject, at, for example, 1 month, 3 months, 6 months, 9 months, 1 year, 2 years, 3 years, 5 years, 10 years, or more, compared to the expected rate of survival of a subject administered an alternative treatment; (iii) a reduction in the prevalence of side effects resulting from the NOS inhibitor composition comprising at least one chemical NOS inhibitor and at least one biological NOS inhibitor compared to a subject administered an alternative treatment; (iv) a reduction in the severity of side effects resulting from the NOS inhibitor composition comprising at least one chemical NOS inhibitor and at least one biological NOS inhibitor compared to a subject administered an alternative treatment; (v) a reduction in the dosage required to achieve an equivalent reduction in tumor volume compared to a subject administered an alternative treatment; (vi) a reduction in the dosage required to achieve an equivalent expected rate of survival of a subject compared to a subject administered an alternative treatment; (vii) a reduction in the likelihood of a recurrence of the NSC-lung cancer in a subject compared to a subject administered an alternative treatment; (viii) a reduction in the likelihood of a metastasis occurring in a subject compared to a subject administered an alternative treatment; (ix) a reduction in the time required to be at a medical facility for treatment compared to a subject administered an alternative treatment; (x) a reduction in the cost associated with treatment compared to a subject administered an alternative treatment; as well as any other improved outcome not expressly recited that would be readily envisaged by one skilled in the art.

In some embodiments, a NOS inhibitor composition, as described herein, may be combined with a conventional therapy, as described herein, to treat, ameliorate, and/or reduce the severity of an NSC-lung cancer or one or more symptoms thereof. In certain embodiments, the NOS inhibitor composition may be administered concurrently with the conventional therapy. In some embodiments, the NOS inhibitor composition may be administered prior to the conventional therapy. In certain embodiments, the NOS inhibitor composition may be administered subsequently to the conventional therapy. Treatment, amelioration, and/or reduction in the severity of an NSC-lung cancer or one or more symptoms thereof may be characterized by an improved outcome compared an alternative treatment. As used herein, the term “alternative treatment,” may define one or more of a conventional therapy, as described herein, a NOS inhibitor composition comprising a single chemical NOS inhibitor, as described herein, and a NOS inhibitor composition comprising a single biological NOS inhibitor, as described herein. For purposes of the disclosure, an “improved outcome” may define any one of the following: (i) a larger reduction in tumor volume and/or mass compared an alternative treatment; (ii) an improvement in the expected rate of survival of a subject, at, for example, 1 month, 3 months, 6 months, 9 months, 1 year, 2 years, 3 years, 5 years, 10 years, or more, compared to the expected rate of survival of a subject administered an alternative treatment; (iii) a reduction in the prevalence of side effects resulting from the NOS inhibitor composition and conventional therapy compared to a subject administered an alternative treatment; (iv) a reduction in the severity of side effects resulting from the NOS inhibitor composition and conventional therapy compared to a subject administered an alternative treatment; (v) a reduction in the dosage required to achieve an equivalent reduction in tumor volume compared to a subject administered an alternative treatment; (vi) a reduction in the dosage required to achieve an equivalent expected rate of survival of a subject compared to a subject administered an alternative treatment; (vii) a reduction in the likelihood of a recurrence of the NSC-lung cancer a in a subject compared to a subject administered an alternative treatment; (viii) a reduction in the likelihood of a metastasis occurring in a subject compared to a subject administered an alternative treatment; (ix) a reduction in the time required to be at a medical facility for treatment compared to a subject administered an alternative treatment; (x) a reduction in the cost associated with treatment compared to a subject administered an alternative treatment; as well as any other improved outcome not expressly recited that would be readily envisaged by one skilled in the art.

In some embodiments, a “conventional therapy,” as that term is used with respect to combined administration with a NOS inhibitor composition, may refer to a conventional chemotherapy. For purposes of the disclosure, a “conventional chemotherapy,” as described herein may be a compound selected from the group consisting of abemaciclib, abiraterone acetate, acalabrutinib, adagrasib, afatinib dimaleate, albumin-bound paclitaxel, albumin-bound rapamycin (Sirolimus Protein-Bound Particles), alectinib, alpelisib, amifostine, aminolevulinic acid hydrochloride, anastrozole, apalutamide, aprepitant, arsenic trioxide, asciminib hydrochloride, avapritinib, axitinib, azacytidine, belinostat, belzutifan, bendamustine hydrochloride, bexarotene, bicalutamide, binimetinib, bleomycin, bleomycin sulfate, bortezomib, bosutinib, brigatinib, busulfan, cabazitaxel, cabozantinib-S-malate, capecitabine, capmatinib hydrochloride, carboplatin, carfilzomib, carmustine, cedazuridine, ceritinib, chlorambucil, cisplatin, cladribine, clofarabine, cobimetinib fumarate, copanlisib hydrochloride, crizotinib, cyclophosphamide, cytarabine, dabrafenib mesylate, dacarbazine, dacomitinib, dactinomycin, darolutamide, dasatinib, daunorubicin hydrochloride, decitabine, defibrotide sodium, degarelix, dexamethasone, dexrazoxane hydrochloride, docetaxel, doxorubicin hydrochloride, duvelisib, elacestrant dihydrochloride, eltrombopag olamine, enasidenib mesylate, encorafenib, entrectinib, enzalutamide, epirubicin hydrochloride, erdafitinib, eribulin mesylate, erlotinib hydrochloride, etoposide, etoposide phosphate, everolimus, exemestane, fedratinib hydrochloride, fludarabine phosphate, fluorouracil, flutamide, folinic acid, fostamatinib disodium, fulvestrant, futibatinib, gefitinib, gemcitabine hydrochloride, gilteritinib fumarate, glasdegib maleate, goserelin acetate, granisetron, granisetron hydrochloride, hydroxyurea, ibrutinib, idarubicin hydrochloride, idelalisib, ifosfamide, imatinib mesylate, imiquimod, infigratinib phosphate, iobenguane I-131, irinotecan hydrochloride, ivosidenib, ixabepilone, ixazomib citrate, lanreotide acetate, lapatinib ditosylate, larotrectinib sulfate, lenalidomide, lenvatinib mesylate, letrozole, leucovorin calcium, leuprolide acetate, liposomal cytarabine, liposomal doxorubicin hydrochloride, liposomal irinotecan hydrochloride, lomustine, lorlatinib, lurbinectedin, Lu-177 dotatate, Lu-177 vipivotide tetraxetan, mechlorethamine hydrochloride, megestrol acetate, melphalan, melphalan hydrochloride, mercaptopurine, mesna, methotrexate, methotrexate sodium, methylnaltrexone bromide, midostaurin, mitomycin, mitoxantrone hydrochloride, mobocertinib succinate, nelarabine, neratinib maleate, netupitant hydrochloride, nilutamide, nilotinib, niraparib tosylate monohydrate, olaparib, olutasidenib, omacetaxine mepesuccinate, ondansetron hydrochloride, osimertinib mesylate, oxaliplatin, paclitaxel, pacritinib citrate, palbociclib, palonosetron hydrochloride, pamidronate disodium, pazopanib hydrochloride, pemetrexed disodium, pemigatinib, pexidartinib hydrochloride, pirtobrutinib, plerixafor, pomalidomide, ponatinib hydrochloride, pralatrexate, pralsetinib, prednisone, procarbazine hydrochloride, propranolol hydrochloride, Ra-223 dichloride, raloxifene hydrochloride, regorafenib, relugolix, ribociclib, ripretinib, rolapitant hydrochloride, romidepsin, rucaparib camsylate, ruxolitinib phosphate, selinexor, selpercatinib, selumetinib sulfate, sonidegib, sorafenib tosylate, sotorasib, sunitinib malate, talazoparib tosylate, talc, tamoxifen citrate, tazemetostat hydrobromide, temozolomide, temsirolimus, tepotinib hydrochloride, thalidomide, thioguanine, thiotepa, tipiracil hydrochloride, tivozanib hydrochloride, topotecan hydrochloride, toremifene, trabectedin, trametinib dimethyl sulfoxide, tretinoin, trifluridine hydrochloride, tucatinib, uridine triacetate, valrubicin, vandetanib, vemurafenib, venetoclax, vinblastine sulfate, vincristine sulfate, vinorelbine tartrate, vismodegib, vorinostat, zanubrutinib, zoledronic acid, and any combination thereof. One skilled in the art would immediately envisage an appropriate “conventional chemotherapy” based on the particular NSC-lung cancer disclosed, as well as characteristics of an NSC-lung cancer subject, such as, for example, the subject's sex, age, stage of the NSC-lung cancer, genotype of the NSC-lung cancer, and the like.

In certain embodiments, a “conventional therapy,” as that term is used with respect to combined administration with a NOS inhibitor composition, may refer to a conventional immunotherapy. For purposes of the disclosure, a “conventional immunotherapy,” as described herein may be an immunotherapy, cell-based therapy, and/or gene-based therapy selected from the group consisting of ado-trastuzumab emtansine, aldesleukin, alemtuzumab, amivantamab-vmjw, asparaginase Erwinia chrysanthemi, asparaginase Erwinia chrysanthemi (recombinant)-rywn, atezolizumab, avelumab, axicabtagene ciloleucel, bevacizumab, blinatumomab, brentuximab vedotin, brexucabtagene autoleucel, calaspargase pegol-mknl, camrelizumab, caplacizumab-yhdp, cemiplimab-rwlc, cetuximab, ciltacabtagene autoleucel, daratumumab, daratumumab and hyaluronidase-fihj, darbepoetin alfa, denileukin diftitox, denosumab, dinutuximab, dostarlimab-gxly, durvalumab, elotuzumab, emapalumab-lzsg, enfortumab vedotin-ejfv, epoetin alfa, fam-trastuzumab deruxtecan-nxki, filgrastim, gemtuzumab ozogamicin, glucarpidase, ibritumomab tiuxetan, idecabtagene vicleucel, inotuzumab ozogamicin, ipilimumab, isatuximab-irfc, lisocabtagene maraleucel, loncastuximab tesirine-lpyl, luspatercept-aamt, margetuximab-cmkb, mirvetuximab soravtansine-gynx, mogamulizumab-kpkc, mosunetuzumab-axgb, moxetumomab pasudotox-tdfk, nadofaragene firadenovec-vncg, naxitamab-gqgk, necitumumab, nivolumab, nivolumab and relatlimab-rmbw, obinutuzumab, ofatumumab, palifermin, panitumumab, pegaspargase, pegfilgrastim, pembrolizumab, pertuzumab, pertuzumab, trastuzumab, and hyaluronidase-zzxf, polatuzumab vedotin-piiq, ramucirumab, rasburicase, ravulizumab-cwvz, recombinant HPV bivalent vaccine, recombinant HPV nonavalent vaccine, recombinant HPV quadrivalent vaccine, recombinant interferon alfa-2b, rituximab, rituximab and hyaluronidase human, romiplostim, ropeginterferon alfa-2b-njft, sacituzumab govitecan-hziy, siltuximab, sintilimab, sipuleucel-T, tafasitamab-cxix, tagraxofusp-erzs, talimogene laherparepvec, tebentafusp-tebn, teclistamab-cqyv, tisagenlecleucel, tislelizumab, tisotumab vedotin-tftv, tocilizumab, toripalimab, trastuzumab, trastuzumab and hyaluronidase-oysk, tremelimumab-actl, ziv-aflibercept, and any combination thereof. One skilled in the art would immediately envisage an appropriate “conventional immunotherapy” based on the particular NSC-lung cancer disclosed, as well as characteristics of an NSC-lung cancer subject, such as, for example, the subject's sex, age, stage of the NSC-lung cancer, genotype of the NSC-lung cancer, and the like.

In certain embodiments, a NOS inhibitor composition, as described herein, may be administered as a suitable dosage form, as described herein, prior to, or alternatively, following a surgery to remove or resect an NSC-lung cancer.

In some embodiments, a NOS inhibitor composition, as described herein, may be administered as a suitable dosage form, as described herein, to restore and/or maintain healthy and/or optimal expression levels of one or more antigens, as described herein, such as, for example, CD15, CD44, CD70, CD133, as well as any CD antigen that has an abnormal expression in a NSC-lung cancer that would be readily envisaged by one skilled in the art, in a subject. In certain embodiments, the subject may have an NSC-lung cancer or one or more symptoms thereof. As used herein, the terms “healthy” and “optimal” refer to expression levels of one or more antigens that would be typical of an equivalent subject that does not have an NSC-lung cancer.

In certain embodiments, a NOS inhibitor composition, as described herein, may be administered as a suitable dosage form, as described herein, to restore and/or maintain healthy and/or optimal expression levels of one or more proteins, as described herein, such as, for example, ALDH1A3, mTOR, p-mTOR, TSC-2, AKT, pAKT, RPS6, p-RPS6, Syp, VEGF, PIGF, as well as any protein or enzyme that has an abnormal expression in a NSC-lung cancer that would be readily envisaged by one skilled in the art, in a subject. In certain embodiments, the subject may have an NSC-lung cancer or one or more symptoms thereof. As used herein, the terms “healthy” and “optimal” refer to expression levels of one or more proteins that would be typical of an equivalent subject that does not have an NSC-lung cancer.

In some embodiments, a NOS inhibitor composition, as described herein, may be administered as a suitable dosage form, as described herein, to restore and/or maintain healthy and/or optimal expression levels of one or more NOS isoforms, as described herein, such as, for example, nNOS, iNOS, and eNOS, in a subject. In certain embodiments, the subject may have an NSC-lung cancer or one or more symptoms thereof. As used herein, the terms “healthy” and “optimal” refer to expression levels of one or more NOS isoforms that would be typical of an equivalent subject that does not have an NSC-lung cancer.

In certain embodiments, a NOS inhibitor composition, as described herein, may be administered as a suitable dosage form, as described herein, to restore and/or maintain healthy and/or optimal levels of one or more NO-indicators, as described herein, such as, for example, 3-Ntyr, NADPH-diaphorase, total nitrite concentration, total nitrate concentration, total nitrite and nitrate concentration, as well as any NO-indicator that has an elevated level in a NSC-lung cancer that would be readily envisaged by one skilled in the art, in a subject. In certain embodiments, the subject may have an NSC-lung cancer or one or more symptoms thereof. As used herein, the terms “healthy” and “optimal” refer to levels of one or more NO-indicators that would be typical of an equivalent subject that does not have an NSC-lung cancer.

In certain embodiments, a NOS inhibitor composition, as described herein, may be administered as a suitable dosage form, as described herein, to treat, ameliorate, and/or reduce the severity of metastatic squamous cell carcinoma of the head and neck (“m-HNSCC”) or one or more symptoms thereof in a subject (“m-HNSCC subject”).

In some embodiments, a NOS inhibitor composition comprising 7-NI and/or a salt thereof may be administered as a suitable dosage form, as described herein, to treat, ameliorate, and/or reduce the severity of M-HNSCC cancer or one or more symptoms thereof in an M-HNSCC cancer subject. In certain embodiments, an nNOS inhibitor composition comprising 7-NI and/or a salt thereof may be administered to an M-HNSCC cancer subject to treat, ameliorate, and/or reduce the severity of M-HNSCC cancer or one or more symptoms thereof in the M-HNSCC cancer subject: (i) via a route selected from the group consisting of buccally, inhalation, intraarterially, intraarticularly, intracardially, intracerebroventricularly, intradermally, intramedullary, intramuscularly, intraperitoneally, intrathecally, intravenously, intraventricularly, nasally, ophthalmically, orally, rectally, sublabially, sublingually, subcutaneously, topically, and transdermally; and/or (ii) with a frequency selected from the group consisting of four times daily, three times daily, twice daily, once daily, once every two days, once every three days, once every four days, once every five days, once every six days, once a week, once every eight days, once every nine days, once every ten days, once every two weeks, once every three weeks, and once a month; and/or (iii) at a dosage (based on subject weight) selected from the group consisting of 0.1 μg/kg, 0.5 μg/kg, 1 μg/kg, 1.5 μg/kg, 2.0 μg/kg, 2.5 μg/kg, 3.0 μg/kg, 3.5 μg/kg, 4.0 μg/kg, 4.5 μg/kg, 5.0 μg/kg, 10 μg/kg, 15 μg/kg, 20 μg/kg, 25 μg/kg, 30 μg/kg, 35 μg/kg, 40 μg/kg, 45 μg/kg, 50 μg/kg, 55 μg/kg, 60 μg/kg, 65 μg/kg, 70 μg/kg, 75 μg/kg, 80 μg/kg, 85 μg/kg, 90 μg/kg, 95 μg/kg, 100 μg/kg, 150 μg/kg, 200 μg/kg, 250 μg/kg, 300 μg/kg, 350 μg/kg, 400 μg/kg, 450 μg/kg, 500 μg/kg, 550 μg/kg, 600 μg/kg, 650 μg/kg, 700 μg/kg, 750 μg/kg, 800 μg/kg, 850 μg/kg, 900 μg/kg, 1 mg/kg, 1.5 mg/kg, 2.0 mg/kg, 2.5 mg/kg, 3 mg/kg, 3.5 mg/kg, 4.0 mg/kg, 4.5 mg/kg, 5 mg/kg, 5.5 mg/kg, 6 mg/kg, 6.5 mg/kg, 7 mg/kg, 7.5 mg/kg, 8 mg/kg, 8.5 mg/kg, 9 mg/kg, 9.5 mg/kg, 10 mg/kg 10.5 mg/kg, 11 mg/kg, 11.5 mg/kg, 12 mg/kg, 12.5 mg/kg, 13 mg/kg, 13.5 mg/kg, 14 mg/kg, 14.5 mg/kg, 15 mg/kg, 16 mg/kg, 17 mg/kg, 18 mg/kg, 19 mg/kg, 20 mg/kg, 21 mg/kg, 22 mg/kg, 23 mg/kg, 24 mg/kg, 25 mg/kg, 30 mg/kg, 35 mg/kg, 40 mg/kg, 45 mg/kg, 50 mg/kg, 55 mg/kg, 60 mg/kg, 65 mg/kg, 70 mg/kg, 75 mg/kg, 80 mg/kg, 85 mg/kg, 90 mg/kg, 95 mg/kg, 100 mg/kg, 105 mg/kg, 110 mg/kg, 115 mg/kg, 120 mg/kg, 125 mg/kg, 130 mg/kg, 135 mg/kg, 140 mg/kg, 145 mg/kg, 150 mg/kg, 155 mg/kg, 160 mg/kg, 165 mg/kg, 170 mg/kg, 175 mg/kg, 180 mg/kg, 185 mg/kg, 190 mg/kg, 195 mg/kg, 200 mg/kg, and any fraction or integer in between any two of the preceding amounts.

In certain embodiments, a NOS inhibitor composition comprising 3-Br-7-NI and/or a salt thereof may be administered as a suitable dosage form, as described herein, to treat, ameliorate, and/or reduce the severity of M-HNSCC cancer or one or more symptoms thereof in an M-HNSCC cancer subject. In some embodiments, an nNOS inhibitor composition comprising 3-Br-7-NI and/or a salt thereof may be administered to an M-HNSCC cancer subject to treat, ameliorate, and/or reduce the severity of M-HNSCC cancer or one or more symptoms thereof in the M-HNSCC cancer subject: (i) via a route selected from the group consisting of buccally, inhalation, intraarterially, intraarticularly, intracardially, intracerebroventricularly, intradermally, intramedullary, intramuscularly, intraperitoneally, intrathecally, intravenously, intraventricularly, nasally, ophthalmically, orally, rectally, sublabially, sublingually, subcutaneously, topically, and transdermally; and/or (ii) with a frequency selected from the group consisting of four times daily, three times daily, twice daily, once daily, once every two days, once every three days, once every four days, once every five days, once every six days, once a week, once every eight days, once every nine days, once every ten days, once every two weeks, once every three weeks, and once a month; and/or (iii) at a dosage (based on subject weight) selected from the group consisting of 0.1 μg/kg, 0.5 μg/kg, 1 μg/kg, 1.5 μg/kg, 2.0 μg/kg, 2.5 μg/kg, 3.0 μg/kg, 3.5 μg/kg, 4.0 μg/kg, 4.5 μg/kg, 5.0 μg/kg, 10 μg/kg, 15 μg/kg, 20 μg/kg, 25 μg/kg, 30 μg/kg, 35 μg/kg, 40 μg/kg, 45 μg/kg, 50 μg/kg, 55 μg/kg, 60 μg/kg, 65 μg/kg, 70 μg/kg, 75 μg/kg, 80 μg/kg, 85 μg/kg, 90 μg/kg, 95 μg/kg, 100 μg/kg, 150 μg/kg, 200 μg/kg, 250 μg/kg, 300 μg/kg, 350 μg/kg, 400 μg/kg, 450 μg/kg, 500 μg/kg, 550 μg/kg, 600 μg/kg, 650 μg/kg, 700 μg/kg, 750 μg/kg, 800 μg/kg, 850 μg/kg, 900 μg/kg, 1 mg/kg, 1.5 mg/kg, 2.0 mg/kg, 2.5 mg/kg, 3 mg/kg, 3.5 mg/kg, 4.0 mg/kg, 4.5 mg/kg, 5 mg/kg, 5.5 mg/kg, 6 mg/kg, 6.5 mg/kg, 7 mg/kg, 7.5 mg/kg, 8 mg/kg, 8.5 mg/kg, 9 mg/kg, 9.5 mg/kg, 10 mg/kg 10.5 mg/kg, 11 mg/kg, 11.5 mg/kg, 12 mg/kg, 12.5 mg/kg, 13 mg/kg, 13.5 mg/kg, 14 mg/kg, 14.5 mg/kg, 15 mg/kg, 16 mg/kg, 17 mg/kg, 18 mg/kg, 19 mg/kg, 20 mg/kg, 21 mg/kg, 22 mg/kg, 23 mg/kg, 24 mg/kg, 25 mg/kg, 30 mg/kg, 35 mg/kg, 40 mg/kg, 45 mg/kg, 50 mg/kg, 55 mg/kg, 60 mg/kg, 65 mg/kg, 70 mg/kg, 75 mg/kg, 80 mg/kg, 85 mg/kg, 90 mg/kg, 95 mg/kg, 100 mg/kg, 105 mg/kg, 110 mg/kg, 115 mg/kg, 120 mg/kg, 125 mg/kg, 130 mg/kg, 135 mg/kg, 140 mg/kg, 145 mg/kg, 150 mg/kg, 155 mg/kg, 160 mg/kg, 165 mg/kg, 170 mg/kg, 175 mg/kg, 180 mg/kg, 185 mg/kg, 190 mg/kg, 195 mg/kg, 200 mg/kg, and any fraction or integer in between any two of the preceding amounts.

In some embodiments, a NOS inhibitor composition comprising L-NPA and/or a salt thereof, such as, for example, L-NPA hydrochloride, may be administered as a suitable dosage form, as described herein, to treat, ameliorate, and/or reduce the severity of M-HNSCC cancer or one or more symptoms thereof in an M-HNSCC cancer subject. In certain embodiments, an nNOS inhibitor composition comprising L-NPA and/or a salt thereof, such as, for example, L-NPA hydrochloride may be administered to an M-HNSCC cancer subject to treat, ameliorate, and/or reduce the severity of M-HNSCC cancer or one or more symptoms thereof in the M-HNSCC cancer subject: (i) via a route selected from the group consisting of buccally, inhalation, intraarterially, intraarticularly, intracardially, intracerebroventricularly, intradermally, intramedullary, intramuscularly, intraperitoneally, intrathecally, intravenously, intraventricularly, nasally, ophthalmically, orally, rectally, sublabially, sublingually, subcutaneously, topically, and transdermally; and/or (ii) with a frequency selected from the group consisting of four times daily, three times daily, twice daily, once daily, once every two days, once every three days, once every four days, once every five days, once every six days, once a week, once every eight days, once every nine days, once every ten days, once every two weeks, once every three weeks, and once a month; and/or (iii) at a dosage (based on subject weight) selected from the group consisting of 0.1 μg/kg, 0.5 μg/kg, 1 μg/kg, 1.5 μg/kg, 2.0 μg/kg, 2.5 μg/kg, 3.0 μg/kg, 3.5 μg/kg, 4.0 μg/kg, 4.5 μg/kg, 5.0 μg/kg, 10 μg/kg, 15 μg/kg, 20 μg/kg, 25 μg/kg, 30 μg/kg, 35 μg/kg, 40 μg/kg, 45 μg/kg, 50 μg/kg, 55 μg/kg, 60 μg/kg, 65 μg/kg, 70 μg/kg, 75 μg/kg, 80 μg/kg, 85 μg/kg, 90 μg/kg, 95 μg/kg, 100 μg/kg, 150 μg/kg, 200 μg/kg, 250 μg/kg, 300 μg/kg, 350 μg/kg, 400 μg/kg, 450 μg/kg, 500 μg/kg, 550 μg/kg, 600 μg/kg, 650 μg/kg, 700 μg/kg, 750 μg/kg, 800 μg/kg, 850 μg/kg, 900 μg/kg, 1 mg/kg, 1.5 mg/kg, 2.0 mg/kg, 2.5 mg/kg, 3 mg/kg, 3.5 mg/kg, 4.0 mg/kg, 4.5 mg/kg, 5 mg/kg, 5.5 mg/kg, 6 mg/kg, 6.5 mg/kg, 7 mg/kg, 7.5 mg/kg, 8 mg/kg, 8.5 mg/kg, 9 mg/kg, 9.5 mg/kg, 10 mg/kg 10.5 mg/kg, 11 mg/kg, 11.5 mg/kg, 12 mg/kg, 12.5 mg/kg, 13 mg/kg, 13.5 mg/kg, 14 mg/kg, 14.5 mg/kg, 15 mg/kg, 16 mg/kg, 17 mg/kg, 18 mg/kg, 19 mg/kg, 20 mg/kg, 21 mg/kg, 22 mg/kg, 23 mg/kg, 24 mg/kg, 25 mg/kg, 30 mg/kg, 35 mg/kg, 40 mg/kg, 45 mg/kg, 50 mg/kg, 55 mg/kg, 60 mg/kg, 65 mg/kg, 70 mg/kg, 75 mg/kg, 80 mg/kg, 85 mg/kg, 90 mg/kg, 95 mg/kg, 100 mg/kg, 105 mg/kg, 110 mg/kg, 115 mg/kg, 120 mg/kg, 125 mg/kg, 130 mg/kg, 135 mg/kg, 140 mg/kg, 145 mg/kg, 150 mg/kg, 155 mg/kg, 160 mg/kg, 165 mg/kg, 170 mg/kg, 175 mg/kg, 180 mg/kg, 185 mg/kg, 190 mg/kg, 195 mg/kg, 200 mg/kg, and any fraction or integer in between any two of the preceding amounts.

In certain embodiments, a NOS inhibitor composition comprising L-NIO and/or a salt thereof, such as, for example, L-NIO dihydrochloride, may be administered as a suitable dosage form, as described herein, to treat, ameliorate, and/or reduce the severity of M-HNSCC cancer or one or more symptoms thereof in an M-HNSCC cancer subject. In some embodiments, an nNOS inhibitor composition comprising L-NIO and/or a salt thereof, such as, for example, L-NIO dihydrochloride, may be administered to an M-HNSCC cancer subject to treat, ameliorate, and/or reduce the severity of M-HNSCC cancer or one or more symptoms thereof in the M-HNSCC cancer subject: (i) via a route selected from the group consisting of buccally, inhalation, intraarterially, intraarticularly, intracardially, intracerebroventricularly, intradermally, intramedullary, intramuscularly, intraperitoneally, intrathecally, intravenously, intraventricularly, nasally, ophthalmically, orally, rectally, sublabially, sublingually, subcutaneously, topically, and transdermally; and/or (ii) with a frequency selected from the group consisting of four times daily, three times daily, twice daily, once daily, once every two days, once every three days, once every four days, once every five days, once every six days, once a week, once every eight days, once every nine days, once every ten days, once every two weeks, once every three weeks, and once a month; and/or (iii) at a dosage (based on subject weight) selected from the group consisting of 0.1 μg/kg, 0.5 μg/kg, 1 μg/kg, 1.5 μg/kg, 2.0 μg/kg, 2.5 μg/kg, 3.0 μg/kg, 3.5 μg/kg, 4.0 μg/kg, 4.5 μg/kg, 5.0 μg/kg, 10 μg/kg, 15 μg/kg, 20 μg/kg, 25 μg/kg, 30 μg/kg, 35 μg/kg, 40 μg/kg, 45 μg/kg, 50 μg/kg, 55 μg/kg, 60 μg/kg, 65 μg/kg, 70 μg/kg, 75 μg/kg, 80 μg/kg, 85 μg/kg, 90 μg/kg, 95 μg/kg, 100 μg/kg, 150 μg/kg, 200 μg/kg, 250 μg/kg, 300 μg/kg, 350 μg/kg, 400 μg/kg, 450 μg/kg, 500 μg/kg, 550 μg/kg, 600 μg/kg, 650 μg/kg, 700 μg/kg, 750 μg/kg, 800 μg/kg, 850 μg/kg, 900 μg/kg, 1 mg/kg, 1.5 mg/kg, 2.0 mg/kg, 2.5 mg/kg, 3 mg/kg, 3.5 mg/kg, 4.0 mg/kg, 4.5 mg/kg, 5 mg/kg, 5.5 mg/kg, 6 mg/kg, 6.5 mg/kg, 7 mg/kg, 7.5 mg/kg, 8 mg/kg, 8.5 mg/kg, 9 mg/kg, 9.5 mg/kg, 10 mg/kg 10.5 mg/kg, 11 mg/kg, 11.5 mg/kg, 12 mg/kg, 12.5 mg/kg, 13 mg/kg, 13.5 mg/kg, 14 mg/kg, 14.5 mg/kg, 15 mg/kg, 16 mg/kg, 17 mg/kg, 18 mg/kg, 19 mg/kg, 20 mg/kg, 21 mg/kg, 22 mg/kg, 23 mg/kg, 24 mg/kg, 25 mg/kg, 30 mg/kg, 35 mg/kg, 40 mg/kg, 45 mg/kg, 50 mg/kg, 55 mg/kg, 60 mg/kg, 65 mg/kg, 70 mg/kg, 75 mg/kg, 80 mg/kg, 85 mg/kg, 90 mg/kg, 95 mg/kg, 100 mg/kg, 105 mg/kg, 110 mg/kg, 115 mg/kg, 120 mg/kg, 125 mg/kg, 130 mg/kg, 135 mg/kg, 140 mg/kg, 145 mg/kg, 150 mg/kg, 155 mg/kg, 160 mg/kg, 165 mg/kg, 170 mg/kg, 175 mg/kg, 180 mg/kg, 185 mg/kg, 190 mg/kg, 195 mg/kg, 200 mg/kg, and any fraction or integer in between any two of the preceding amounts.

In some embodiments, a NOS inhibitor composition comprising ARL-17477 and/or a salt thereof, such as, for example, ARL-17477 dihydrochloride, may be administered as a suitable dosage form, as described herein, to treat, ameliorate, and/or reduce the severity of M-HNSCC cancer or one or more symptoms thereof in an M-HNSCC cancer subject. In certain embodiments, an nNOS inhibitor composition comprising ARL-17477 and/or a salt thereof, such as, for example, ARL-17477 dihydrochloride, may be administered to an M-HNSCC cancer subject to treat, ameliorate, and/or reduce the severity of M-HNSCC cancer or one or more symptoms thereof in the M-HNSCC cancer subject: (i) via a route selected from the group consisting of buccally, inhalation, intraarterially, intraarticularly, intracardially, intracerebroventricularly, intradermally, intramedullary, intramuscularly, intraperitoneally, intrathecally, intravenously, intraventricularly, nasally, ophthalmically, orally, rectally, sublabially, sublingually, subcutaneously, topically, and transdermally; and/or (ii) with a frequency selected from the group consisting of four times daily, three times daily, twice daily, once daily, once every two days, once every three days, once every four days, once every five days, once every six days, once a week, once every eight days, once every nine days, once every ten days, once every two weeks, once every three weeks, and once a month; and/or (iii) at a dosage (based on subject weight) selected from the group consisting of 0.1 μg/kg, 0.5 μg/kg, 1 μg/kg, 1.5 μg/kg, 2.0 μg/kg, 2.5 μg/kg, 3.0 μg/kg, 3.5 μg/kg, 4.0 μg/kg, 4.5 μg/kg, 5.0 μg/kg, 10 μg/kg, 15 μg/kg, 20 μg/kg, 25 μg/kg, 30 μg/kg, 35 μg/kg, 40 μg/kg, 45 μg/kg, 50 μg/kg, 55 μg/kg, 60 μg/kg, 65 μg/kg, 70 μg/kg, 75 μg/kg, 80 μg/kg, 85 μg/kg, 90 μg/kg, 95 μg/kg, 100 μg/kg, 150 μg/kg, 200 μg/kg, 250 μg/kg, 300 μg/kg, 350 μg/kg, 400 μg/kg, 450 μg/kg, 500 μg/kg, 550 μg/kg, 600 μg/kg, 650 μg/kg, 700 μg/kg, 750 μg/kg, 800 μg/kg, 850 μg/kg, 900 μg/kg, 1 mg/kg, 1.5 mg/kg, 2.0 mg/kg, 2.5 mg/kg, 3 mg/kg, 3.5 mg/kg, 4.0 mg/kg, 4.5 mg/kg, 5 mg/kg, 5.5 mg/kg, 6 mg/kg, 6.5 mg/kg, 7 mg/kg, 7.5 mg/kg, 8 mg/kg, 8.5 mg/kg, 9 mg/kg, 9.5 mg/kg, 10 mg/kg 10.5 mg/kg, 11 mg/kg, 11.5 mg/kg, 12 mg/kg, 12.5 mg/kg, 13 mg/kg, 13.5 mg/kg, 14 mg/kg, 14.5 mg/kg, 15 mg/kg, 16 mg/kg, 17 mg/kg, 18 mg/kg, 19 mg/kg, 20 mg/kg, 21 mg/kg, 22 mg/kg, 23 mg/kg, 24 mg/kg, 25 mg/kg, 30 mg/kg, 35 mg/kg, 40 mg/kg, 45 mg/kg, 50 mg/kg, 55 mg/kg, 60 mg/kg, 65 mg/kg, 70 mg/kg, 75 mg/kg, 80 mg/kg, 85 mg/kg, 90 mg/kg, 95 mg/kg, 100 mg/kg, 105 mg/kg, 110 mg/kg, 115 mg/kg, 120 mg/kg, 125 mg/kg, 130 mg/kg, 135 mg/kg, 140 mg/kg, 145 mg/kg, 150 mg/kg, 155 mg/kg, 160 mg/kg, 165 mg/kg, 170 mg/kg, 175 mg/kg, 180 mg/kg, 185 mg/kg, 190 mg/kg, 195 mg/kg, 200 mg/kg, and any fraction or integer in between any two of the preceding amounts.

In certain embodiments, a NOS inhibitor composition comprising L-NNA and/or a salt thereof may be administered as a suitable dosage form, as described herein, to treat, ameliorate, and/or reduce the severity of M-HNSCC cancer or one or more symptoms thereof in an M-HNSCC cancer subject. In some embodiments, an nNOS inhibitor composition comprising L-NNA and/or a salt thereof may be administered to an M-HNSCC cancer subject to treat, ameliorate, and/or reduce the severity of M-HNSCC cancer or one or more symptoms thereof in the M-HNSCC cancer subject: (i) via a route selected from the group consisting of buccally, inhalation, intraarterially, intraarticularly, intracardially, intracerebroventricularly, intradermally, intramedullary, intramuscularly, intraperitoneally, intrathecally, intravenously, intraventricularly, nasally, ophthalmically, orally, rectally, sublabially, sublingually, subcutaneously, topically, and transdermally; and/or (ii) with a frequency selected from the group consisting of four times daily, three times daily, twice daily, once daily, once every two days, once every three days, once every four days, once every five days, once every six days, once a week, once every eight days, once every nine days, once every ten days, once every two weeks, once every three weeks, and once a month; and/or (iii) at a dosage (based on subject weight) selected from the group consisting of 0.1 μg/kg, 0.5 μg/kg, 1 μg/kg, 1.5 μg/kg, 2.0 μg/kg, 2.5 μg/kg, 3.0 μg/kg, 3.5 μg/kg, 4.0 μg/kg, 4.5 μg/kg, 5.0 μg/kg, 10 μg/kg, 15 μg/kg, 20 μg/kg, 25 μg/kg, 30 μg/kg, 35 μg/kg, 40 μg/kg, 45 μg/kg, 50 μg/kg, 55 μg/kg, 60 μg/kg, 65 μg/kg, 70 μg/kg, 75 μg/kg, 80 μg/kg, 85 μg/kg, 90 μg/kg, 95 μg/kg, 100 μg/kg, 150 μg/kg, 200 μg/kg, 250 μg/kg, 300 μg/kg, 350 μg/kg, 400 μg/kg, 450 μg/kg, 500 μg/kg, 550 μg/kg, 600 μg/kg, 650 μg/kg, 700 μg/kg, 750 μg/kg, 800 μg/kg, 850 μg/kg, 900 μg/kg, 1 mg/kg, 1.5 mg/kg, 2.0 mg/kg, 2.5 mg/kg, 3 mg/kg, 3.5 mg/kg, 4.0 mg/kg, 4.5 mg/kg, 5 mg/kg, 5.5 mg/kg, 6 mg/kg, 6.5 mg/kg, 7 mg/kg, 7.5 mg/kg, 8 mg/kg, 8.5 mg/kg, 9 mg/kg, 9.5 mg/kg, 10 mg/kg 10.5 mg/kg, 11 mg/kg, 11.5 mg/kg, 12 mg/kg, 12.5 mg/kg, 13 mg/kg, 13.5 mg/kg, 14 mg/kg, 14.5 mg/kg, 15 mg/kg, 16 mg/kg, 17 mg/kg, 18 mg/kg, 19 mg/kg, 20 mg/kg, 21 mg/kg, 22 mg/kg, 23 mg/kg, 24 mg/kg, 25 mg/kg, 30 mg/kg, 35 mg/kg, 40 mg/kg, 45 mg/kg, 50 mg/kg, 55 mg/kg, 60 mg/kg, 65 mg/kg, 70 mg/kg, 75 mg/kg, 80 mg/kg, 85 mg/kg, 90 mg/kg, 95 mg/kg, 100 mg/kg, 105 mg/kg, 110 mg/kg, 115 mg/kg, 120 mg/kg, 125 mg/kg, 130 mg/kg, 135 mg/kg, 140 mg/kg, 145 mg/kg, 150 mg/kg, 155 mg/kg, 160 mg/kg, 165 mg/kg, 170 mg/kg, 175 mg/kg, 180 mg/kg, 185 mg/kg, 190 mg/kg, 195 mg/kg, 200 mg/kg, and any fraction or integer in between any two of the preceding amounts.

In some embodiments, a NOS inhibitor composition comprising L-NAME and/or a salt thereof, such as, for example, L-NAME hydrochloride, may be administered as a suitable dosage form, as described herein, to treat, ameliorate, and/or reduce the severity of M-HNSCC cancer or one or more symptoms thereof in an M-HNSCC cancer subject. In certain embodiments, an nNOS inhibitor composition comprising L-NAME and/or a salt thereof, such as, for example, L-NAME hydrochloride, may be administered to an M-HNSCC cancer subject to treat, ameliorate, and/or reduce the severity of M-HNSCC cancer or one or more symptoms thereof in the M-HNSCC cancer subject: (i) via a route selected from the group consisting of buccally, inhalation, intraarterially, intraarticularly, intracardially, intracerebroventricularly, intradermally, intramedullary, intramuscularly, intraperitoneally, intrathecally, intravenously, intraventricularly, nasally, ophthalmically, orally, rectally, sublabially, sublingually, subcutaneously, topically, and transdermally; and/or (ii) with a frequency selected from the group consisting of four times daily, three times daily, twice daily, once daily, once every two days, once every three days, once every four days, once every five days, once every six days, once a week, once every eight days, once every nine days, once every ten days, once every two weeks, once every three weeks, and once a month; and/or (iii) at a dosage (based on subject weight) selected from the group consisting of 0.1 μg/kg, 0.5 μg/kg, 1 μg/kg, 1.5 μg/kg, 2.0 μg/kg, 2.5 μg/kg, 3.0 μg/kg, 3.5 μg/kg, 4.0 μg/kg, 4.5 μg/kg, 5.0 μg/kg, 10 μg/kg, 15 μg/kg, 20 μg/kg, 25 μg/kg, 30 μg/kg, 35 μg/kg, 40 μg/kg, 45 μg/kg, 50 μg/kg, 55 μg/kg, 60 μg/kg, 65 μg/kg, 70 μg/kg, 75 μg/kg, 80 μg/kg, 85 μg/kg, 90 μg/kg, 95 μg/kg, 100 μg/kg, 150 μg/kg, 200 μg/kg, 250 μg/kg, 300 μg/kg, 350 μg/kg, 400 μg/kg, 450 μg/kg, 500 μg/kg, 550 μg/kg, 600 μg/kg, 650 μg/kg, 700 μg/kg, 750 μg/kg, 800 μg/kg, 850 μg/kg, 900 μg/kg, 1 mg/kg, 1.5 mg/kg, 2.0 mg/kg, 2.5 mg/kg, 3 mg/kg, 3.5 mg/kg, 4.0 mg/kg, 4.5 mg/kg, 5 mg/kg, 5.5 mg/kg, 6 mg/kg, 6.5 mg/kg, 7 mg/kg, 7.5 mg/kg, 8 mg/kg, 8.5 mg/kg, 9 mg/kg, 9.5 mg/kg, 10 mg/kg 10.5 mg/kg, 11 mg/kg, 11.5 mg/kg, 12 mg/kg, 12.5 mg/kg, 13 mg/kg, 13.5 mg/kg, 14 mg/kg, 14.5 mg/kg, 15 mg/kg, 16 mg/kg, 17 mg/kg, 18 mg/kg, 19 mg/kg, 20 mg/kg, 21 mg/kg, 22 mg/kg, 23 mg/kg, 24 mg/kg, 25 mg/kg, 30 mg/kg, 35 mg/kg, 40 mg/kg, 45 mg/kg, 50 mg/kg, 55 mg/kg, 60 mg/kg, 65 mg/kg, 70 mg/kg, 75 mg/kg, 80 mg/kg, 85 mg/kg, 90 mg/kg, 95 mg/kg, 100 mg/kg, 105 mg/kg, 110 mg/kg, 115 mg/kg, 120 mg/kg, 125 mg/kg, 130 mg/kg, 135 mg/kg, 140 mg/kg, 145 mg/kg, 150 mg/kg, 155 mg/kg, 160 mg/kg, 165 mg/kg, 170 mg/kg, 175 mg/kg, 180 mg/kg, 185 mg/kg, 190 mg/kg, 195 mg/kg, 200 mg/kg, and any fraction or integer in between any two of the preceding amounts.

In certain embodiments, a NOS inhibitor composition comprising L-NMMA and/or a salt thereof, such as, for example, L-NMMA acetate, may be administered as a suitable dosage form, as described herein, to treat, ameliorate, and/or reduce the severity of M-HNSCC cancer or one or more symptoms thereof in an M-HNSCC cancer subject. In some embodiments, an nNOS inhibitor composition comprising L-NMMA and/or a salt thereof, such as, for example, L-NMMA acetate, may be administered to an M-HNSCC cancer subject to treat, ameliorate, and/or reduce the severity of M-HNSCC cancer or one or more symptoms thereof in the M-HNSCC cancer subject: (i) via a route selected from the group consisting of buccally, inhalation, intraarterially, intraarticularly, intracardially, intracerebroventricularly, intradermally, intramedullary, intramuscularly, intraperitoneally, intrathecally, intravenously, intraventricularly, nasally, ophthalmically, orally, rectally, sublabially, sublingually, subcutaneously, topically, and transdermally; and/or (ii) with a frequency selected from the group consisting of four times daily, three times daily, twice daily, once daily, once every two days, once every three days, once every four days, once every five days, once every six days, once a week, once every eight days, once every nine days, once every ten days, once every two weeks, once every three weeks, and once a month; and/or (iii) at a dosage (based on subject weight) selected from the group consisting of 0.1 μg/kg, 0.5 μg/kg, 1 μg/kg, 1.5 μg/kg, 2.0 μg/kg, 2.5 μg/kg, 3.0 μg/kg, 3.5 μg/kg, 4.0 μg/kg, 4.5 μg/kg, 5.0 μg/kg, 10 μg/kg, 15 μg/kg, 20 μg/kg, 25 μg/kg, 30 μg/kg, 35 μg/kg, 40 μg/kg, 45 μg/kg, 50 μg/kg, 55 μg/kg, 60 μg/kg, 65 μg/kg, 70 μg/kg, 75 μg/kg, 80 μg/kg, 85 μg/kg, 90 μg/kg, 95 μg/kg, 100 μg/kg, 150 μg/kg, 200 μg/kg, 250 μg/kg, 300 μg/kg, 350 μg/kg, 400 μg/kg, 450 μg/kg, 500 μg/kg, 550 μg/kg, 600 μg/kg, 650 μg/kg, 700 μg/kg, 750 μg/kg, 800 μg/kg, 850 μg/kg, 900 μg/kg, 1 mg/kg, 1.5 mg/kg, 2.0 mg/kg, 2.5 mg/kg, 3 mg/kg, 3.5 mg/kg, 4.0 mg/kg, 4.5 mg/kg, 5 mg/kg, 5.5 mg/kg, 6 mg/kg, 6.5 mg/kg, 7 mg/kg, 7.5 mg/kg, 8 mg/kg, 8.5 mg/kg, 9 mg/kg, 9.5 mg/kg, 10 mg/kg 10.5 mg/kg, 11 mg/kg, 11.5 mg/kg, 12 mg/kg, 12.5 mg/kg, 13 mg/kg, 13.5 mg/kg, 14 mg/kg, 14.5 mg/kg, 15 mg/kg, 16 mg/kg, 17 mg/kg, 18 mg/kg, 19 mg/kg, 20 mg/kg, 21 mg/kg, 22 mg/kg, 23 mg/kg, 24 mg/kg, 25 mg/kg, 30 mg/kg, 35 mg/kg, 40 mg/kg, 45 mg/kg, 50 mg/kg, 55 mg/kg, 60 mg/kg, 65 mg/kg, 70 mg/kg, 75 mg/kg, 80 mg/kg, 85 mg/kg, 90 mg/kg, 95 mg/kg, 100 mg/kg, 105 mg/kg, 110 mg/kg, 115 mg/kg, 120 mg/kg, 125 mg/kg, 130 mg/kg, 135 mg/kg, 140 mg/kg, 145 mg/kg, 150 mg/kg, 155 mg/kg, 160 mg/kg, 165 mg/kg, 170 mg/kg, 175 mg/kg, 180 mg/kg, 185 mg/kg, 190 mg/kg, 195 mg/kg, 200 mg/kg, and any fraction or integer in between any two of the preceding amounts.

In some embodiments, a NOS inhibitor composition comprising GW-274150 and/or a salt thereof, such as, for example, GW-274150 phosphate, may be administered as a suitable dosage form, as described herein, to treat, ameliorate, and/or reduce the severity of M-HNSCC cancer or one or more symptoms thereof in an M-HNSCC cancer subject. In certain embodiments, an nNOS inhibitor composition comprising GW-274150 and/or a salt thereof, such as, for example, GW-274150 phosphate, may be administered to an M-HNSCC cancer subject to treat, ameliorate, and/or reduce the severity of M-HNSCC cancer or one or more symptoms thereof in the M-HNSCC cancer subject: (i) via a route selected from the group consisting of buccally, inhalation, intraarterially, intraarticularly, intracardially, intracerebroventricularly, intradermally, intramedullary, intramuscularly, intraperitoneally, intrathecally, intravenously, intraventricularly, nasally, ophthalmically, orally, rectally, sublabially, sublingually, subcutaneously, topically, and transdermally; and/or (ii) with a frequency selected from the group consisting of four times daily, three times daily, twice daily, once daily, once every two days, once every three days, once every four days, once every five days, once every six days, once a week, once every eight days, once every nine days, once every ten days, once every two weeks, once every three weeks, and once a month; and/or (iii) at a dosage (based on subject weight) selected from the group consisting of 0.1 μg/kg, 0.5 μg/kg, 1 μg/kg, 1.5 μg/kg, 2.0 μg/kg, 2.5 μg/kg, 3.0 μg/kg, 3.5 μg/kg, 4.0 μg/kg, 4.5 μg/kg, 5.0 μg/kg, 10 μg/kg, 15 μg/kg, 20 μg/kg, 25 μg/kg, 30 μg/kg, 35 μg/kg, 40 μg/kg, 45 μg/kg, 50 μg/kg, 55 μg/kg, 60 μg/kg, 65 μg/kg, 70 μg/kg, 75 μg/kg, 80 μg/kg, 85 μg/kg, 90 μg/kg, 95 μg/kg, 100 μg/kg, 150 μg/kg, 200 μg/kg, 250 μg/kg, 300 μg/kg, 350 μg/kg, 400 μg/kg, 450 μg/kg, 500 μg/kg, 550 μg/kg, 600 μg/kg, 650 μg/kg, 700 μg/kg, 750 μg/kg, 800 μg/kg, 850 μg/kg, 900 μg/kg, 1 mg/kg, 1.5 mg/kg, 2.0 mg/kg, 2.5 mg/kg, 3 mg/kg, 3.5 mg/kg, 4.0 mg/kg, 4.5 mg/kg, 5 mg/kg, 5.5 mg/kg, 6 mg/kg, 6.5 mg/kg, 7 mg/kg, 7.5 mg/kg, 8 mg/kg, 8.5 mg/kg, 9 mg/kg, 9.5 mg/kg, 10 mg/kg 10.5 mg/kg, 11 mg/kg, 11.5 mg/kg, 12 mg/kg, 12.5 mg/kg, 13 mg/kg, 13.5 mg/kg, 14 mg/kg, 14.5 mg/kg, 15 mg/kg, 16 mg/kg, 17 mg/kg, 18 mg/kg, 19 mg/kg, 20 mg/kg, 21 mg/kg, 22 mg/kg, 23 mg/kg, 24 mg/kg, 25 mg/kg, 30 mg/kg, 35 mg/kg, 40 mg/kg, 45 mg/kg, 50 mg/kg, 55 mg/kg, 60 mg/kg, 65 mg/kg, 70 mg/kg, 75 mg/kg, 80 mg/kg, 85 mg/kg, 90 mg/kg, 95 mg/kg, 100 mg/kg, 105 mg/kg, 110 mg/kg, 115 mg/kg, 120 mg/kg, 125 mg/kg, 130 mg/kg, 135 mg/kg, 140 mg/kg, 145 mg/kg, 150 mg/kg, 155 mg/kg, 160 mg/kg, 165 mg/kg, 170 mg/kg, 175 mg/kg, 180 mg/kg, 185 mg/kg, 190 mg/kg, 195 mg/kg, 200 mg/kg, and any fraction or integer in between any two of the preceding amounts.

In certain embodiments, a NOS inhibitor composition comprising L-NIL and/or a salt thereof, such as, for example, L-NIL hydrochloride, may be administered as a suitable dosage form, as described herein, to treat, ameliorate, and/or reduce the severity of M-HNSCC cancer or one or more symptoms thereof in an M-HNSCC cancer subject. In some embodiments, an nNOS inhibitor composition comprising L-NIL and/or a salt thereof, such as, for example, L-NIL hydrochloride, may be administered to an M-HNSCC cancer subject to treat, ameliorate, and/or reduce the severity of M-HNSCC cancer or one or more symptoms thereof in the M-HNSCC cancer subject: (i) via a route selected from the group consisting of buccally, inhalation, intraarterially, intraarticularly, intracardially, intracerebroventricularly, intradermally, intramedullary, intramuscularly, intraperitoneally, intrathecally, intravenously, intraventricularly, nasally, ophthalmically, orally, rectally, sublabially, sublingually, subcutaneously, topically, and transdermally; and/or (ii) with a frequency selected from the group consisting of four times daily, three times daily, twice daily, once daily, once every two days, once every three days, once every four days, once every five days, once every six days, once a week, once every eight days, once every nine days, once every ten days, once every two weeks, once every three weeks, and once a month; and/or (iii) at a dosage (based on subject weight) selected from the group consisting of 0.1 μg/kg, 0.5 μg/kg, 1 μg/kg, 1.5 μg/kg, 2.0 μg/kg, 2.5 μg/kg, 3.0 μg/kg, 3.5 μg/kg, 4.0 μg/kg, 4.5 μg/kg, 5.0 μg/kg, 10 μg/kg, 15 μg/kg, 20 μg/kg, 25 μg/kg, 30 μg/kg, 35 μg/kg, 40 μg/kg, 45 μg/kg, 50 μg/kg, 55 μg/kg, 60 μg/kg, 65 μg/kg, 70 μg/kg, 75 μg/kg, 80 μg/kg, 85 μg/kg, 90 μg/kg, 95 μg/kg, 100 μg/kg, 150 μg/kg, 200 μg/kg, 250 μg/kg, 300 μg/kg, 350 μg/kg, 400 μg/kg, 450 μg/kg, 500 μg/kg, 550 μg/kg, 600 μg/kg, 650 μg/kg, 700 μg/kg, 750 μg/kg, 800 μg/kg, 850 μg/kg, 900 μg/kg, 1 mg/kg, 1.5 mg/kg, 2.0 mg/kg, 2.5 mg/kg, 3 mg/kg, 3.5 mg/kg, 4.0 mg/kg, 4.5 mg/kg, 5 mg/kg, 5.5 mg/kg, 6 mg/kg, 6.5 mg/kg, 7 mg/kg, 7.5 mg/kg, 8 mg/kg, 8.5 mg/kg, 9 mg/kg, 9.5 mg/kg, 10 mg/kg 10.5 mg/kg, 11 mg/kg, 11.5 mg/kg, 12 mg/kg, 12.5 mg/kg, 13 mg/kg, 13.5 mg/kg, 14 mg/kg, 14.5 mg/kg, 15 mg/kg, 16 mg/kg, 17 mg/kg, 18 mg/kg, 19 mg/kg, 20 mg/kg, 21 mg/kg, 22 mg/kg, 23 mg/kg, 24 mg/kg, 25 mg/kg, 30 mg/kg, 35 mg/kg, 40 mg/kg, 45 mg/kg, 50 mg/kg, 55 mg/kg, 60 mg/kg, 65 mg/kg, 70 mg/kg, 75 mg/kg, 80 mg/kg, 85 mg/kg, 90 mg/kg, 95 mg/kg, 100 mg/kg, 105 mg/kg, 110 mg/kg, 115 mg/kg, 120 mg/kg, 125 mg/kg, 130 mg/kg, 135 mg/kg, 140 mg/kg, 145 mg/kg, 150 mg/kg, 155 mg/kg, 160 mg/kg, 165 mg/kg, 170 mg/kg, 175 mg/kg, 180 mg/kg, 185 mg/kg, 190 mg/kg, 195 mg/kg, 200 mg/kg, and any fraction or integer in between any two of the preceding amounts.

In some embodiments, a NOS inhibitor composition comprising 1400W and/or a salt thereof, such as, for example, 1400W dihydrochloride, may be administered as a suitable dosage form, as described herein, to treat, ameliorate, and/or reduce the severity of M-HNSCC cancer or one or more symptoms thereof in an M-HNSCC cancer subject. In certain embodiments, an nNOS inhibitor composition comprising 1400W and/or a salt thereof, such as, for example, 1400W dihydrochloride, may be administered to an M-HNSCC cancer subject to treat, ameliorate, and/or reduce the severity of M-HNSCC cancer or one or more symptoms thereof in the M-HNSCC cancer subject: (i) via a route selected from the group consisting of buccally, inhalation, intraarterially, intraarticularly, intracardially, intracerebroventricularly, intradermally, intramedullary, intramuscularly, intraperitoneally, intrathecally, intravenously, intraventricularly, nasally, ophthalmically, orally, rectally, sublabially, sublingually, subcutaneously, topically, and transdermally; and/or (ii) with a frequency selected from the group consisting of four times daily, three times daily, twice daily, once daily, once every two days, once every three days, once every four days, once every five days, once every six days, once a week, once every eight days, once every nine days, once every ten days, once every two weeks, once every three weeks, and once a month; and/or (iii) at a dosage (based on subject weight) selected from the group consisting of 0.1 μg/kg, 0.5 μg/kg, 1 μg/kg, 1.5 μg/kg, 2.0 μg/kg, 2.5 μg/kg, 3.0 μg/kg, 3.5 μg/kg, 4.0 μg/kg, 4.5 μg/kg, 5.0 μg/kg, 10 μg/kg, 15 μg/kg, 20 μg/kg, 25 μg/kg, 30 μg/kg, 35 μg/kg, 40 μg/kg, 45 μg/kg, 50 μg/kg, 55 μg/kg, 60 μg/kg, 65 μg/kg, 70 μg/kg, 75 μg/kg, 80 μg/kg, 85 μg/kg, 90 μg/kg, 95 μg/kg, 100 μg/kg, 150 μg/kg, 200 μg/kg, 250 μg/kg, 300 μg/kg, 350 μg/kg, 400 μg/kg, 450 μg/kg, 500 μg/kg, 550 μg/kg, 600 μg/kg, 650 μg/kg, 700 μg/kg, 750 μg/kg, 800 μg/kg, 850 μg/kg, 900 μg/kg, 1 mg/kg, 1.5 mg/kg, 2.0 mg/kg, 2.5 mg/kg, 3 mg/kg, 3.5 mg/kg, 4.0 mg/kg, 4.5 mg/kg, 5 mg/kg, 5.5 mg/kg, 6 mg/kg, 6.5 mg/kg, 7 mg/kg, 7.5 mg/kg, 8 mg/kg, 8.5 mg/kg, 9 mg/kg, 9.5 mg/kg, 10 mg/kg 10.5 mg/kg, 11 mg/kg, 11.5 mg/kg, 12 mg/kg, 12.5 mg/kg, 13 mg/kg, 13.5 mg/kg, 14 mg/kg, 14.5 mg/kg, 15 mg/kg, 16 mg/kg, 17 mg/kg, 18 mg/kg, 19 mg/kg, 20 mg/kg, 21 mg/kg, 22 mg/kg, 23 mg/kg, 24 mg/kg, 25 mg/kg, 30 mg/kg, 35 mg/kg, 40 mg/kg, 45 mg/kg, 50 mg/kg, 55 mg/kg, 60 mg/kg, 65 mg/kg, 70 mg/kg, 75 mg/kg, 80 mg/kg, 85 mg/kg, 90 mg/kg, 95 mg/kg, 100 mg/kg, 105 mg/kg, 110 mg/kg, 115 mg/kg, 120 mg/kg, 125 mg/kg, 130 mg/kg, 135 mg/kg, 140 mg/kg, 145 mg/kg, 150 mg/kg, 155 mg/kg, 160 mg/kg, 165 mg/kg, 170 mg/kg, 175 mg/kg, 180 mg/kg, 185 mg/kg, 190 mg/kg, 195 mg/kg, 200 mg/kg, and any fraction or integer in between any two of the preceding amounts.

In certain embodiments, a NOS inhibitor composition comprising OKN-007 and/or a salt thereof, such as, for example, OKN-007 sodium, may be administered as a suitable dosage form, as described herein, to treat, ameliorate, and/or reduce the severity of M-HNSCC cancer or one or more symptoms thereof in an M-HNSCC cancer subject. In some embodiments, an nNOS inhibitor composition comprising OKN-007 and/or a salt thereof, such as, for example, OKN-007 sodium, may be administered to an M-HNSCC cancer subject to treat, ameliorate, and/or reduce the severity of M-HNSCC cancer or one or more symptoms thereof in the M-HNSCC cancer subject: (i) via a route selected from the group consisting of buccally, inhalation, intraarterially, intraarticularly, intracardially, intracerebroventricularly, intradermally, intramedullary, intramuscularly, intraperitoneally, intrathecally, intravenously, intraventricularly, nasally, ophthalmically, orally, rectally, sublabially, sublingually, subcutaneously, topically, and transdermally; and/or (ii) with a frequency selected from the group consisting of four times daily, three times daily, twice daily, once daily, once every two days, once every three days, once every four days, once every five days, once every six days, once a week, once every eight days, once every nine days, once every ten days, once every two weeks, once every three weeks, and once a month; and/or (iii) at a dosage (based on subject weight) selected from the group consisting of 0.1 μg/kg, 0.5 μg/kg, 1 μg/kg, 1.5 μg/kg, 2.0 μg/kg, 2.5 μg/kg, 3.0 μg/kg, 3.5 μg/kg, 4.0 μg/kg, 4.5 μg/kg, 5.0 μg/kg, 10 μg/kg, 15 μg/kg, 20 μg/kg, 25 μg/kg, 30 μg/kg, 35 μg/kg, 40 μg/kg, 45 μg/kg, 50 μg/kg, 55 μg/kg, 60 μg/kg, 65 μg/kg, 70 μg/kg, 75 μg/kg, 80 μg/kg, 85 μg/kg, 90 μg/kg, 95 μg/kg, 100 μg/kg, 150 μg/kg, 200 μg/kg, 250 μg/kg, 300 μg/kg, 350 μg/kg, 400 μg/kg, 450 μg/kg, 500 μg/kg, 550 μg/kg, 600 μg/kg, 650 μg/kg, 700 μg/kg, 750 μg/kg, 800 μg/kg, 850 μg/kg, 900 μg/kg, 1 mg/kg, 1.5 mg/kg, 2.0 mg/kg, 2.5 mg/kg, 3 mg/kg, 3.5 mg/kg, 4.0 mg/kg, 4.5 mg/kg, 5 mg/kg, 5.5 mg/kg, 6 mg/kg, 6.5 mg/kg, 7 mg/kg, 7.5 mg/kg, 8 mg/kg, 8.5 mg/kg, 9 mg/kg, 9.5 mg/kg, 10 mg/kg 10.5 mg/kg, 11 mg/kg, 11.5 mg/kg, 12 mg/kg, 12.5 mg/kg, 13 mg/kg, 13.5 mg/kg, 14 mg/kg, 14.5 mg/kg, 15 mg/kg, 16 mg/kg, 17 mg/kg, 18 mg/kg, 19 mg/kg, 20 mg/kg, 21 mg/kg, 22 mg/kg, 23 mg/kg, 24 mg/kg, 25 mg/kg, 30 mg/kg, 35 mg/kg, 40 mg/kg, 45 mg/kg, 50 mg/kg, 55 mg/kg, 60 mg/kg, 65 mg/kg, 70 mg/kg, 75 mg/kg, 80 mg/kg, 85 mg/kg, 90 mg/kg, 95 mg/kg, 100 mg/kg, 105 mg/kg, 110 mg/kg, 115 mg/kg, 120 mg/kg, 125 mg/kg, 130 mg/kg, 135 mg/kg, 140 mg/kg, 145 mg/kg, 150 mg/kg, 155 mg/kg, 160 mg/kg, 165 mg/kg, 170 mg/kg, 175 mg/kg, 180 mg/kg, 185 mg/kg, 190 mg/kg, 195 mg/kg, 200 mg/kg, and any fraction or integer in between any two of the preceding amounts.

In some embodiments, a NOS inhibitor composition comprising BYK-191023 and/or a salt thereof, such as, for example, BYK-191023 dihydrochloride, may be administered as a suitable dosage form, as described herein, to treat, ameliorate, and/or reduce the severity of M-HNSCC cancer or one or more symptoms thereof in an M-HNSCC cancer subject. In certain embodiments, an nNOS inhibitor composition comprising BYK-191023 and/or a salt thereof, such as, for example, BYK-191023 dihydrochloride, may be administered to an M-HNSCC cancer subject to treat, ameliorate, and/or reduce the severity of M-HNSCC cancer or one or more symptoms thereof in the M-HNSCC cancer subject: (i) via a route selected from the group consisting of buccally, inhalation, intraarterially, intraarticularly, intracardially, intracerebroventricularly, intradermally, intramedullary, intramuscularly, intraperitoneally, intrathecally, intravenously, intraventricularly, nasally, ophthalmically, orally, rectally, sublabially, sublingually, subcutaneously, topically, and transdermally; and/or (ii) with a frequency selected from the group consisting of four times daily, three times daily, twice daily, once daily, once every two days, once every three days, once every four days, once every five days, once every six days, once a week, once every eight days, once every nine days, once every ten days, once every two weeks, once every three weeks, and once a month; and/or (iii) at a dosage (based on subject weight) selected from the group consisting of 0.1 μg/kg, 0.5 μg/kg, 1 μg/kg, 1.5 μg/kg, 2.0 μg/kg, 2.5 μg/kg, 3.0 μg/kg, 3.5 μg/kg, 4.0 μg/kg, 4.5 μg/kg, 5.0 μg/kg, 10 μg/kg, 15 μg/kg, 20 μg/kg, 25 μg/kg, 30 μg/kg, 35 μg/kg, 40 μg/kg, 45 μg/kg, 50 μg/kg, 55 μg/kg, 60 μg/kg, 65 μg/kg, 70 μg/kg, 75 μg/kg, 80 μg/kg, 85 μg/kg, 90 μg/kg, 95 μg/kg, 100 μg/kg, 150 μg/kg, 200 μg/kg, 250 μg/kg, 300 μg/kg, 350 μg/kg, 400 μg/kg, 450 μg/kg, 500 μg/kg, 550 μg/kg, 600 μg/kg, 650 μg/kg, 700 μg/kg, 750 μg/kg, 800 μg/kg, 850 μg/kg, 900 μg/kg, 1 mg/kg, 1.5 mg/kg, 2.0 mg/kg, 2.5 mg/kg, 3 mg/kg, 3.5 mg/kg, 4.0 mg/kg, 4.5 mg/kg, 5 mg/kg, 5.5 mg/kg, 6 mg/kg, 6.5 mg/kg, 7 mg/kg, 7.5 mg/kg, 8 mg/kg, 8.5 mg/kg, 9 mg/kg, 9.5 mg/kg, 10 mg/kg 10.5 mg/kg, 11 mg/kg, 11.5 mg/kg, 12 mg/kg, 12.5 mg/kg, 13 mg/kg, 13.5 mg/kg, 14 mg/kg, 14.5 mg/kg, 15 mg/kg, 16 mg/kg, 17 mg/kg, 18 mg/kg, 19 mg/kg, 20 mg/kg, 21 mg/kg, 22 mg/kg, 23 mg/kg, 24 mg/kg, 25 mg/kg, 30 mg/kg, 35 mg/kg, 40 mg/kg, 45 mg/kg, 50 mg/kg, 55 mg/kg, 60 mg/kg, 65 mg/kg, 70 mg/kg, 75 mg/kg, 80 mg/kg, 85 mg/kg, 90 mg/kg, 95 mg/kg, 100 mg/kg, 105 mg/kg, 110 mg/kg, 115 mg/kg, 120 mg/kg, 125 mg/kg, 130 mg/kg, 135 mg/kg, 140 mg/kg, 145 mg/kg, 150 mg/kg, 155 mg/kg, 160 mg/kg, 165 mg/kg, 170 mg/kg, 175 mg/kg, 180 mg/kg, 185 mg/kg, 190 mg/kg, 195 mg/kg, 200 mg/kg, and any fraction or integer in between any two of the preceding amounts.

In certain embodiments, a NOS inhibitor composition comprising CM544 and/or a salt thereof, such as, for example, CM544 dihydrochloride, may be administered as a suitable dosage form, as described herein, to treat, ameliorate, and/or reduce the severity of M-HNSCC cancer or one or more symptoms thereof in an M-HNSCC cancer subject. In some embodiments, an nNOS inhibitor composition comprising CM544 and/or a salt thereof, such as, for example, CM544 dihydrochloride, may be administered to an M-HNSCC cancer subject to treat, ameliorate, and/or reduce the severity of M-HNSCC cancer or one or more symptoms thereof in the M-HNSCC cancer subject: (i) via a route selected from the group consisting of buccally, inhalation, intraarterially, intraarticularly, intracardially, intracerebroventricularly, intradermally, intramedullary, intramuscularly, intraperitoneally, intrathecally, intravenously, intraventricularly, nasally, ophthalmically, orally, rectally, sublabially, sublingually, subcutaneously, topically, and transdermally; and/or (ii) with a frequency selected from the group consisting of four times daily, three times daily, twice daily, once daily, once every two days, once every three days, once every four days, once every five days, once every six days, once a week, once every eight days, once every nine days, once every ten days, once every two weeks, once every three weeks, and once a month; and/or (iii) at a dosage (based on subject weight) selected from the group consisting of 0.1 μg/kg, 0.5 μg/kg, 1 μg/kg, 1.5 μg/kg, 2.0 μg/kg, 2.5 μg/kg, 3.0 μg/kg, 3.5 μg/kg, 4.0 μg/kg, 4.5 μg/kg, 5.0 μg/kg, 10 μg/kg, 15 μg/kg, 20 μg/kg, 25 μg/kg, 30 μg/kg, 35 μg/kg, 40 μg/kg, 45 μg/kg, 50 μg/kg, 55 μg/kg, 60 μg/kg, 65 μg/kg, 70 μg/kg, 75 μg/kg, 80 μg/kg, 85 μg/kg, 90 μg/kg, 95 μg/kg, 100 μg/kg, 150 μg/kg, 200 μg/kg, 250 μg/kg, 300 μg/kg, 350 μg/kg, 400 μg/kg, 450 μg/kg, 500 μg/kg, 550 μg/kg, 600 μg/kg, 650 μg/kg, 700 μg/kg, 750 μg/kg, 800 μg/kg, 850 μg/kg, 900 μg/kg, 1 mg/kg, 1.5 mg/kg, 2.0 mg/kg, 2.5 mg/kg, 3 mg/kg, 3.5 mg/kg, 4.0 mg/kg, 4.5 mg/kg, 5 mg/kg, 5.5 mg/kg, 6 mg/kg, 6.5 mg/kg, 7 mg/kg, 7.5 mg/kg, 8 mg/kg, 8.5 mg/kg, 9 mg/kg, 9.5 mg/kg, 10 mg/kg 10.5 mg/kg, 11 mg/kg, 11.5 mg/kg, 12 mg/kg, 12.5 mg/kg, 13 mg/kg, 13.5 mg/kg, 14 mg/kg, 14.5 mg/kg, 15 mg/kg, 16 mg/kg, 17 mg/kg, 18 mg/kg, 19 mg/kg, 20 mg/kg, 21 mg/kg, 22 mg/kg, 23 mg/kg, 24 mg/kg, 25 mg/kg, 30 mg/kg, 35 mg/kg, 40 mg/kg, 45 mg/kg, 50 mg/kg, 55 mg/kg, 60 mg/kg, 65 mg/kg, 70 mg/kg, 75 mg/kg, 80 mg/kg, 85 mg/kg, 90 mg/kg, 95 mg/kg, 100 mg/kg, 105 mg/kg, 110 mg/kg, 115 mg/kg, 120 mg/kg, 125 mg/kg, 130 mg/kg, 135 mg/kg, 140 mg/kg, 145 mg/kg, 150 mg/kg, 155 mg/kg, 160 mg/kg, 165 mg/kg, 170 mg/kg, 175 mg/kg, 180 mg/kg, 185 mg/kg, 190 mg/kg, 195 mg/kg, 200 mg/kg, and any fraction or integer in between any two of the preceding amounts.

In certain embodiments, an nNOS inhibitor composition comprising one or more dsRNAs each comprising at least 30 base pairs, may be administered as a suitable dosage form, as described herein, to treat, ameliorate, and/or reduce the severity of M-HNSCC cancer or one or more symptoms thereof in an M-HNSCC cancer subject. In some embodiments, an nNOS inhibitor composition comprising one or more dsRNAs, each comprising at least 30 base pairs, may be administered to an M-HNSCC cancer subject to treat, ameliorate, and/or reduce the severity of M-HNSCC cancer or one or more symptoms thereof in the M-HNSCC cancer subject: (i) via a route selected from the group consisting of buccally, inhalation, intraarterially, intraarticularly, intracardially, intracerebroventricularly, intradermally, intramedullary, intramuscularly, intraperitoneally, intrathecally, intravenously, intraventricularly, nasally, ophthalmically, orally, rectally, sublabially, sublingually, subcutaneously, topically, and transdermally; and/or (ii) with a frequency selected from the group consisting of four times daily, three times daily, twice daily, once daily, once every two days, once every three days, once every four days, once every five days, once every six days, once a week, once every eight days, once every nine days, once every ten days, once every two weeks, once every three weeks, and once a month; and/or (iii) at a dosage (based on subject weight) selected from the group consisting of 0.1 μg/kg, 0.5 μg/kg, 1 μg/kg, 1.5 μg/kg, 2.0 μg/kg, 2.5 μg/kg, 3.0 μg/kg, 3.5 μg/kg, 4.0 μg/kg, 4.5 μg/kg, 5.0 μg/kg, 10 μg/kg, 15 μg/kg, 20 μg/kg, 25 μg/kg, 30 μg/kg, 35 μg/kg, 40 μg/kg, 45 μg/kg, 50 μg/kg, 55 μg/kg, 60 μg/kg, 65 μg/kg, 70 μg/kg, 75 μg/kg, 80 μg/kg, 85 μg/kg, 90 μg/kg, 95 μg/kg, 100 μg/kg, 150 μg/kg, 200 μg/kg, 250 μg/kg, 300 μg/kg, 350 μg/kg, 400 μg/kg, 450 μg/kg, 500 μg/kg, 550 μg/kg, 600 μg/kg, 650 μg/kg, 700 μg/kg, 750 μg/kg, 800 μg/kg, 850 μg/kg, 900 μg/kg, 1 mg/kg, 1.5 mg/kg, 2.0 mg/kg, 2.5 mg/kg, 3 mg/kg, 3.5 mg/kg, 4.0 mg/kg, 4.5 mg/kg, 5 mg/kg, 5.5 mg/kg, 6 mg/kg, 6.5 mg/kg, 7 mg/kg, 7.5 mg/kg, 8 mg/kg, 8.5 mg/kg, 9 mg/kg, 9.5 mg/kg, 10 mg/kg 10.5 mg/kg, 11 mg/kg, 11.5 mg/kg, 12 mg/kg, 12.5 mg/kg, 13 mg/kg, 13.5 mg/kg, 14 mg/kg, 14.5 mg/kg, 15 mg/kg, 16 mg/kg, 17 mg/kg, 18 mg/kg, 19 mg/kg, 20 mg/kg, 21 mg/kg, 22 mg/kg, 23 mg/kg, 24 mg/kg, 25 mg/kg, 30 mg/kg, 35 mg/kg, 40 mg/kg, 45 mg/kg, 50 mg/kg, 55 mg/kg, 60 mg/kg, 65 mg/kg, 70 mg/kg, 75 mg/kg, 80 mg/kg, 85 mg/kg, 90 mg/kg, 95 mg/kg, 100 mg/kg, 105 mg/kg, 110 mg/kg, 115 mg/kg, 120 mg/kg, 125 mg/kg, 130 mg/kg, 135 mg/kg, 140 mg/kg, 145 mg/kg, 150 mg/kg, 155 mg/kg, 160 mg/kg, 165 mg/kg, 170 mg/kg, 175 mg/kg, 180 mg/kg, 185 mg/kg, 190 mg/kg, 195 mg/kg, 200 mg/kg, and any fraction or integer in between any two of the preceding amounts.

In some embodiments, an nNOS inhibitor composition comprising one or more siRNAs, may be administered as a suitable dosage form, as described herein, to treat, ameliorate, and/or reduce the severity of M-HNSCC cancer or one or more symptoms thereof in an M-HNSCC cancer subject. In certain embodiments, an nNOS inhibitor composition comprising one or more siRNAs selected from the group consisting of SEQ ID NO. 4, SEQ ID NO. 5, SEQ ID NO. 6, SEQ ID NO. 7, SEQ ID NO. 8, and any combination thereof, may be administered as a suitable dosage form, as described herein, to treat, ameliorate, and/or reduce the severity of M-HNSCC cancer or one or more symptoms thereof in an M-HNSCC cancer subject. In some embodiments, an nNOS inhibitor composition comprising one or more siRNAs selected from the group consisting of SEQ ID NO. 4, SEQ ID NO. 5, SEQ ID NO. 6, SEQ ID NO. 7, SEQ ID NO. 8, and any combination thereof, may be administered to an M-HNSCC cancer subject to treat, ameliorate, and/or reduce the severity of M-HNSCC cancer or one or more symptoms thereof in the M-HNSCC cancer subject: (i) via a route selected from the group consisting of buccally, inhalation, intraarterially, intraarticularly, intracardially, intracerebroventricularly, intradermally, intramedullary, intramuscularly, intraperitoneally, intrathecally, intravenously, intraventricularly, nasally, ophthalmically, orally, rectally, sublabially, sublingually, subcutaneously, topically, and transdermally; and/or (ii) with a frequency selected from the group consisting of four times daily, three times daily, twice daily, once daily, once every two days, once every three days, once every four days, once every five days, once every six days, once a week, once every eight days, once every nine days, once every ten days, once every two weeks, once every three weeks, and once a month; and/or (iii) at a dosage (based on subject weight) selected from the group consisting of 0.1 μg/kg, 0.5 μg/kg, 1 μg/kg, 1.5 μg/kg, 2.0 μg/kg, 2.5 μg/kg, 3.0 μg/kg, 3.5 μg/kg, 4.0 μg/kg, 4.5 μg/kg, 5.0 μg/kg, 10 μg/kg, 15 μg/kg, 20 μg/kg, 25 μg/kg, 30 μg/kg, 35 μg/kg, 40 μg/kg, 45 μg/kg, 50 μg/kg, 55 μg/kg, 60 μg/kg, 65 μg/kg, 70 μg/kg, 75 μg/kg, 80 μg/kg, 85 μg/kg, 90 μg/kg, 95 μg/kg, 100 μg/kg, 150 μg/kg, 200 μg/kg, 250 μg/kg, 300 μg/kg, 350 μg/kg, 400 μg/kg, 450 μg/kg, 500 μg/kg, 550 μg/kg, 600 μg/kg, 650 μg/kg, 700 μg/kg, 750 μg/kg, 800 μg/kg, 850 μg/kg, 900 μg/kg, 1 mg/kg, 1.5 mg/kg, 2.0 mg/kg, 2.5 mg/kg, 3 mg/kg, 3.5 mg/kg, 4.0 mg/kg, 4.5 mg/kg, 5 mg/kg, 5.5 mg/kg, 6 mg/kg, 6.5 mg/kg, 7 mg/kg, 7.5 mg/kg, 8 mg/kg, 8.5 mg/kg, 9 mg/kg, 9.5 mg/kg, 10 mg/kg 10.5 mg/kg, 11 mg/kg, 11.5 mg/kg, 12 mg/kg, 12.5 mg/kg, 13 mg/kg, 13.5 mg/kg, 14 mg/kg, 14.5 mg/kg, 15 mg/kg, 16 mg/kg, 17 mg/kg, 18 mg/kg, 19 mg/kg, 20 mg/kg, 21 mg/kg, 22 mg/kg, 23 mg/kg, 24 mg/kg, 25 mg/kg, 30 mg/kg, 35 mg/kg, 40 mg/kg, 45 mg/kg, 50 mg/kg, 55 mg/kg, 60 mg/kg, 65 mg/kg, 70 mg/kg, 75 mg/kg, 80 mg/kg, 85 mg/kg, 90 mg/kg, 95 mg/kg, 100 mg/kg, 105 mg/kg, 110 mg/kg, 115 mg/kg, 120 mg/kg, 125 mg/kg, 130 mg/kg, 135 mg/kg, 140 mg/kg, 145 mg/kg, 150 mg/kg, 155 mg/kg, 160 mg/kg, 165 mg/kg, 170 mg/kg, 175 mg/kg, 180 mg/kg, 185 mg/kg, 190 mg/kg, 195 mg/kg, 200 mg/kg, and any fraction or integer in between any two of the preceding amounts.

In some embodiments, an nNOS inhibitor composition comprising one or more shRNAs, may be administered as a suitable dosage form, as described herein, to treat, ameliorate, and/or reduce the severity of M-HNSCC cancer or one or more symptoms thereof in an M-HNSCC cancer subject. In certain embodiments, an nNOS inhibitor composition comprising one or more shRNAs selected from the group consisting of SEQ ID NO. 9, SEQ ID NO. 10, SEQ ID NO. 11, SEQ ID NO. 12, SEQ ID NO. 13, SEQ ID NO. 14, and any combination thereof, may be administered as a suitable dosage form, as described herein, to treat, ameliorate, and/or reduce the severity of M-HNSCC cancer or one or more symptoms thereof in an M-HNSCC cancer subject. In some embodiments, an nNOS inhibitor composition comprising one or more shRNAs selected from the group consisting of SEQ ID NO. 9, SEQ ID NO. 10, SEQ ID NO. 11, SEQ ID NO. 12, SEQ ID NO. 13, SEQ ID NO. 14, and any combination thereof, may be administered to an M-HNSCC cancer subject to treat, ameliorate, and/or reduce the severity of M-HNSCC cancer or one or more symptoms thereof in the M-HNSCC cancer subject: (i) via a route selected from the group consisting of buccally, inhalation, intraarterially, intraarticularly, intracardially, intracerebroventricularly, intradermally, intramedullary, intramuscularly, intraperitoneally, intrathecally, intravenously, intraventricularly, nasally, ophthalmically, orally, rectally, sublabially, sublingually, subcutaneously, topically, and transdermally; and/or (ii) with a frequency selected from the group consisting of four times daily, three times daily, twice daily, once daily, once every two days, once every three days, once every four days, once every five days, once every six days, once a week, once every eight days, once every nine days, once every ten days, once every two weeks, once every three weeks, and once a month; and/or (iii) at a dosage (based on subject weight) selected from the group consisting of 0.1 μg/kg, 0.5 μg/kg, 1 μg/kg, 1.5 μg/kg, 2.0 μg/kg, 2.5 μg/kg, 3.0 μg/kg, 3.5 μg/kg, 4.0 μg/kg, 4.5 μg/kg, 5.0 μg/kg, 10 μg/kg, 15 μg/kg, 20 μg/kg, 25 μg/kg, 30 μg/kg, 35 μg/kg, 40 μg/kg, 45 μg/kg, 50 μg/kg, 55 μg/kg, 60 μg/kg, 65 μg/kg, 70 μg/kg, 75 μg/kg, 80 μg/kg, 85 μg/kg, 90 μg/kg, 95 μg/kg, 100 μg/kg, 150 μg/kg, 200 μg/kg, 250 μg/kg, 300 μg/kg, 350 μg/kg, 400 μg/kg, 450 μg/kg, 500 μg/kg, 550 μg/kg, 600 μg/kg, 650 μg/kg, 700 μg/kg, 750 μg/kg, 800 μg/kg, 850 μg/kg, 900 μg/kg, 1 mg/kg, 1.5 mg/kg, 2.0 mg/kg, 2.5 mg/kg, 3 mg/kg, 3.5 mg/kg, 4.0 mg/kg, 4.5 mg/kg, 5 mg/kg, 5.5 mg/kg, 6 mg/kg, 6.5 mg/kg, 7 mg/kg, 7.5 mg/kg, 8 mg/kg, 8.5 mg/kg, 9 mg/kg, 9.5 mg/kg, 10 mg/kg 10.5 mg/kg, 11 mg/kg, 11.5 mg/kg, 12 mg/kg, 12.5 mg/kg, 13 mg/kg, 13.5 mg/kg, 14 mg/kg, 14.5 mg/kg, 15 mg/kg, 16 mg/kg, 17 mg/kg, 18 mg/kg, 19 mg/kg, 20 mg/kg, 21 mg/kg, 22 mg/kg, 23 mg/kg, 24 mg/kg, 25 mg/kg, 30 mg/kg, 35 mg/kg, 40 mg/kg, 45 mg/kg, 50 mg/kg, 55 mg/kg, 60 mg/kg, 65 mg/kg, 70 mg/kg, 75 mg/kg, 80 mg/kg, 85 mg/kg, 90 mg/kg, 95 mg/kg, 100 mg/kg, 105 mg/kg, 110 mg/kg, 115 mg/kg, 120 mg/kg, 125 mg/kg, 130 mg/kg, 135 mg/kg, 140 mg/kg, 145 mg/kg, 150 mg/kg, 155 mg/kg, 160 mg/kg, 165 mg/kg, 170 mg/kg, 175 mg/kg, 180 mg/kg, 185 mg/kg, 190 mg/kg, 195 mg/kg, 200 mg/kg, and any fraction or integer in between any two of the preceding amounts.

In some embodiments, an nNOS inhibitor composition comprising one or more miRNAs, may be administered as a suitable dosage form, as described herein, to treat, ameliorate, and/or reduce the severity of M-HNSCC cancer or one or more symptoms thereof in an M-HNSCC cancer subject. In certain embodiments, an nNOS inhibitor composition comprising one or more miRNAs selected from the group consisting of miR-146a, hsa-miR-146a-5p, hsa-miR-1273f, hsa-miR-143-3p, hsa-miR-147a, hsa-miR-3622a-5p, hsa-miR-3911, hsa-miR-4269, hsa-miR-4708-5p, hsa-miR-4710, hsa-miR-4770, hsa-miR-4792, hsa-miR-574-5p, hsa-miR-6088, hsa-miR-644a, hsa-miR-6715b-5p, hsa-miR-6768-5p, hsa-miR-6867-5p, hsa-miR-7855-5p, hsa-miR-8485, and any combination thereof, may be administered as a suitable dosage form, as described herein, to treat, ameliorate, and/or reduce the severity of M-HNSCC cancer or one or more symptoms thereof in an M-HNSCC cancer subject. In some embodiments, an nNOS inhibitor composition comprising one or more miRNAs selected from the group consisting of rniR-146a, hsa-miR-146a-5p, hsa-miR-1273f, hsa-miR-143-3p, hsa-miR-147a, hsa-miR-3622a-5p, hsa-miR-3911, hsa-miR-4269, hsa-miR-4708-5p, hsa-miR-4710, hsa-miR-4770, hsa-miR-4792, hsa-miR-574-5p, hsa-miR-6088, hsa-miR-644a, hsa-miR-6715b-5p, hsa-miR-6768-5p, hsa-miR-6867-5p, hsa-miR-7855-5p, hsa-miR-8485, and any combination thereof, may be administered to an M-HNSCC cancer subject to treat, ameliorate, and/or reduce the severity of M-HNSCC cancer or one or more symptoms thereof in the M-HNSCC cancer subject: (i) via a route selected from the group consisting of buccally, inhalation, intraarterially, intraarticularly, intracardially, intracerebroventricularly, intradermally, intramedullary, intramuscularly, intraperitoneally, intrathecally, intravenously, intraventricularly, nasally, ophthalmically, orally, rectally, sublabially, sublingually, subcutaneously, topically, and transdermally; and/or (ii) with a frequency selected from the group consisting of four times daily, three times daily, twice daily, once daily, once every two days, once every three days, once every four days, once every five days, once every six days, once a week, once every eight days, once every nine days, once every ten days, once every two weeks, once every three weeks, and once a month; and/or (iii) at a dosage (based on subject weight) selected from the group consisting of 0.1 μg/kg, 0.5 μg/kg, 1 μg/kg, 1.5 μg/kg, 2.0 μg/kg, 2.5 μg/kg, 3.0 μg/kg, 3.5 μg/kg, 4.0 μg/kg, 4.5 μg/kg, 5.0 μg/kg, 10 μg/kg, 15 μg/kg, 20 μg/kg, 25 μg/kg, 30 μg/kg, 35 μg/kg, 40 μg/kg, 45 μg/kg, 50 μg/kg, 55 μg/kg, 60 μg/kg, 65 μg/kg, 70 μg/kg, 75 μg/kg, 80 μg/kg, 85 μg/kg, 90 μg/kg, 95 μg/kg, 100 μg/kg, 150 μg/kg, 200 μg/kg, 250 μg/kg, 300 μg/kg, 350 μg/kg, 400 μg/kg, 450 μg/kg, 500 μg/kg, 550 μg/kg, 600 μg/kg, 650 μg/kg, 700 μg/kg, 750 μg/kg, 800 μg/kg, 850 μg/kg, 900 μg/kg, 1 mg/kg, 1.5 mg/kg, 2.0 mg/kg, 2.5 mg/kg, 3 mg/kg, 3.5 mg/kg, 4.0 mg/kg, 4.5 mg/kg, 5 mg/kg, 5.5 mg/kg, 6 mg/kg, 6.5 mg/kg, 7 mg/kg, 7.5 mg/kg, 8 mg/kg, 8.5 mg/kg, 9 mg/kg, 9.5 mg/kg, 10 mg/kg 10.5 mg/kg, 11 mg/kg, 11.5 mg/kg, 12 mg/kg, 12.5 mg/kg, 13 mg/kg, 13.5 mg/kg, 14 mg/kg, 14.5 mg/kg, 15 mg/kg, 16 mg/kg, 17 mg/kg, 18 mg/kg, 19 mg/kg, 20 mg/kg, 21 mg/kg, 22 mg/kg, 23 mg/kg, 24 mg/kg, 25 mg/kg, 30 mg/kg, 35 mg/kg, 40 mg/kg, 45 mg/kg, 50 mg/kg, 55 mg/kg, 60 mg/kg, 65 mg/kg, 70 mg/kg, 75 mg/kg, 80 mg/kg, 85 mg/kg, 90 mg/kg, 95 mg/kg, 100 mg/kg, 105 mg/kg, 110 mg/kg, 115 mg/kg, 120 mg/kg, 125 mg/kg, 130 mg/kg, 135 mg/kg, 140 mg/kg, 145 mg/kg, 150 mg/kg, 155 mg/kg, 160 mg/kg, 165 mg/kg, 170 mg/kg, 175 mg/kg, 180 mg/kg, 185 mg/kg, 190 mg/kg, 195 mg/kg, 200 mg/kg, and any fraction or integer in between any two of the preceding amounts.

In some embodiments, an nNOS inhibitor composition comprising one or more CRISPR/Cas systems, may be administered as a suitable dosage form, as described herein, to treat, ameliorate, and/or reduce the severity of M-HNSCC cancer or one or more symptoms thereof in an M-HNSCC cancer subject. In certain embodiments, an nNOS inhibitor composition comprising one or more CRISPR/Cas systems comprising an endonuclease selected from the group consisting of Cas1, Cas2, Cas3, Cas4, Cas5, Cas6, Cas7, Cas8a, Cas8b, Cas8c, Cas9, Cas10, Cas10d, Cas12, Cas12a, Cas12b, Cas12c, Cas12d, Cas12e, Cas12f, Cas12g, Cas12h, Cas12i, Cas12k, C2c4, C2c8, C2c9, Cas13, Cas13a, Cas13b, Cas13c, Cas13d, Cas13x.1, Cse1, Cse2, Csy1, Csy2, Csy3, GSU0054, Csm2, Cmr5, Csx10, Csx11, Csf1, and Csn2, may be administered as a suitable dosage form, as described herein, to treat, ameliorate, and/or reduce the severity of M-HNSCC cancer or one or more symptoms thereof in an M-HNSCC cancer subject. In some embodiments, an nNOS inhibitor composition comprising one or more CRISPR/Cas systems comprising an endonuclease selected from the group consisting of Cas1, Cas2, Cas3, Cas4, Cas5, Cas6, Cas7, Cas8a, Cas8b, Cas8c, Cas9, Cas10, Cas10d, Cas12, Cas12a, Cas12b, Cas12c, Cas12d, Cas12e, Cas12f, Cas12g, Cas12h, Cas12i, Cas12k, C2c4, C2c8, C2c9, Cas13, Cas13a, Cas13b, Cas13c, Cas13d, Cas13x.1, Cse1, Cse2, Csy1, Csy2, Csy3, GSU0054, Csm2, Cmr5, Csx10, Csx11, Csf1, and Csn2, may be administered to an M-HNSCC cancer subject to treat, ameliorate, and/or reduce the severity of M-HNSCC cancer or one or more symptoms thereof in the M-HNSCC cancer subject: (i) via a route selected from the group consisting of buccally, inhalation, intraarterially, intraarticularly, intracardially, intracerebroventricularly, intradermally, intramedullary, intramuscularly, intraperitoneally, intrathecally, intravenously, intraventricularly, nasally, ophthalmically, orally, rectally, sublabially, sublingually, subcutaneously, topically, and transdermally; and/or (ii) with a frequency selected from the group consisting of four times daily, three times daily, twice daily, once daily, once every two days, once every three days, once every four days, once every five days, once every six days, once a week, once every eight days, once every nine days, once every ten days, once every two weeks, once every three weeks, and once a month; and/or (iii) at a dosage (based on subject weight) selected from the group consisting of 0.1 μg/kg, 0.5 μg/kg, 1 μg/kg, 1.5 μg/kg, 2.0 μg/kg, 2.5 μg/kg, 3.0 μg/kg, 3.5 μg/kg, 4.0 μg/kg, 4.5 μg/kg, 5.0 μg/kg, 10 μg/kg, 15 μg/kg, 20 μg/kg, 25 μg/kg, 30 μg/kg, 35 μg/kg, 40 μg/kg, 45 μg/kg, 50 μg/kg, 55 μg/kg, 60 μg/kg, 65 μg/kg, 70 μg/kg, 75 μg/kg, 80 μg/kg, 85 μg/kg, 90 μg/kg, 95 μg/kg, 100 μg/kg, 150 μg/kg, 200 μg/kg, 250 μg/kg, 300 μg/kg, 350 μg/kg, 400 μg/kg, 450 μg/kg, 500 μg/kg, 550 μg/kg, 600 μg/kg, 650 μg/kg, 700 μg/kg, 750 μg/kg, 800 μg/kg, 850 μg/kg, 900 μg/kg, 1 mg/kg, 1.5 mg/kg, 2.0 mg/kg, 2.5 mg/kg, 3 mg/kg, 3.5 mg/kg, 4.0 mg/kg, 4.5 mg/kg, 5 mg/kg, 5.5 mg/kg, 6 mg/kg, 6.5 mg/kg, 7 mg/kg, 7.5 mg/kg, 8 mg/kg, 8.5 mg/kg, 9 mg/kg, 9.5 mg/kg, 10 mg/kg 10.5 mg/kg, 11 mg/kg, 11.5 mg/kg, 12 mg/kg, 12.5 mg/kg, 13 mg/kg, 13.5 mg/kg, 14 mg/kg, 14.5 mg/kg, 15 mg/kg, 16 mg/kg, 17 mg/kg, 18 mg/kg, 19 mg/kg, 20 mg/kg, 21 mg/kg, 22 mg/kg, 23 mg/kg, 24 mg/kg, 25 mg/kg, 30 mg/kg, 35 mg/kg, 40 mg/kg, 45 mg/kg, 50 mg/kg, 55 mg/kg, 60 mg/kg, 65 mg/kg, 70 mg/kg, 75 mg/kg, 80 mg/kg, 85 mg/kg, 90 mg/kg, 95 mg/kg, 100 mg/kg, 105 mg/kg, 110 mg/kg, 115 mg/kg, 120 mg/kg, 125 mg/kg, 130 mg/kg, 135 mg/kg, 140 mg/kg, 145 mg/kg, 150 mg/kg, 155 mg/kg, 160 mg/kg, 165 mg/kg, 170 mg/kg, 175 mg/kg, 180 mg/kg, 185 mg/kg, 190 mg/kg, 195 mg/kg, 200 mg/kg, and any fraction or integer in between any two of the preceding amounts.

In some embodiments, an nNOS inhibitor composition comprising one or more antibodies, may be administered as a suitable dosage form, as described herein, to treat, ameliorate, and/or reduce the severity of M-HNSCC cancer or one or more symptoms thereof in an M-HNSCC cancer subject. In certain embodiments, an nNOS inhibitor composition comprising one or more antibodies selected from the group consisting of 37-2800 (from ThermoFisher), EP1855Y (from abeam), EPR 24351-6 (from abeam), and any combination thereof, may be administered as a suitable dosage form, as described herein, to treat, ameliorate, and/or reduce the severity of M-HNSCC cancer or one or more symptoms thereof in an M-HNSCC cancer subject. In some embodiments, an nNOS inhibitor composition comprising one or more antibodies selected from the group consisting of 37-2800 (from ThermoFisher), EP1855Y (from abeam), EPR 24351-6 (from abeam), and any combination thereof, may be administered to an M-HNSCC cancer subject to treat, ameliorate, and/or reduce the severity of M-HNSCC cancer or one or more symptoms thereof in the M-HNSCC cancer subject: (i) via a route selected from the group consisting of buccally, inhalation, intraarterially, intraarticularly, intracardially, intracerebroventricularly, intradermally, intramedullary, intramuscularly, intraperitoneally, intrathecally, intravenously, intraventricularly, nasally, ophthalmically, orally, rectally, sublabially, sublingually, subcutaneously, topically, and transdermally; and/or (ii) with a frequency selected from the group consisting of four times daily, three times daily, twice daily, once daily, once every two days, once every three days, once every four days, once every five days, once every six days, once a week, once every eight days, once every nine days, once every ten days, once every two weeks, once every three weeks, and once a month; and/or (iii) at a dosage (based on subject weight) selected from the group consisting of 0.1 μg/kg, 0.5 μg/kg, 1 μg/kg, 1.5 μg/kg, 2.0 μg/kg, 2.5 μg/kg, 3.0 μg/kg, 3.5 μg/kg, 4.0 μg/kg, 4.5 μg/kg, 5.0 μg/kg, 10 μg/kg, 15 μg/kg, 20 μg/kg, 25 μg/kg, 30 μg/kg, 35 μg/kg, 40 μg/kg, 45 μg/kg, 50 μg/kg, 55 μg/kg, 60 μg/kg, 65 μg/kg, 70 μg/kg, 75 μg/kg, 80 μg/kg, 85 μg/kg, 90 μg/kg, 95 μg/kg, 100 μg/kg, 150 μg/kg, 200 μg/kg, 250 μg/kg, 300 μg/kg, 350 μg/kg, 400 μg/kg, 450 μg/kg, 500 μg/kg, 550 μg/kg, 600 μg/kg, 650 μg/kg, 700 μg/kg, 750 μg/kg, 800 μg/kg, 850 μg/kg, 900 μg/kg, 1 mg/kg, 1.5 mg/kg, 2.0 mg/kg, 2.5 mg/kg, 3 mg/kg, 3.5 mg/kg, 4.0 mg/kg, 4.5 mg/kg, 5 mg/kg, 5.5 mg/kg, 6 mg/kg, 6.5 mg/kg, 7 mg/kg, 7.5 mg/kg, 8 mg/kg, 8.5 mg/kg, 9 mg/kg, 9.5 mg/kg, 10 mg/kg 10.5 mg/kg, 11 mg/kg, 11.5 mg/kg, 12 mg/kg, 12.5 mg/kg, 13 mg/kg, 13.5 mg/kg, 14 mg/kg, 14.5 mg/kg, 15 mg/kg, 16 mg/kg, 17 mg/kg, 18 mg/kg, 19 mg/kg, 20 mg/kg, 21 mg/kg, 22 mg/kg, 23 mg/kg, 24 mg/kg, 25 mg/kg, 30 mg/kg, 35 mg/kg, 40 mg/kg, 45 mg/kg, 50 mg/kg, 55 mg/kg, 60 mg/kg, 65 mg/kg, 70 mg/kg, 75 mg/kg, 80 mg/kg, 85 mg/kg, 90 mg/kg, 95 mg/kg, 100 mg/kg, 105 mg/kg, 110 mg/kg, 115 mg/kg, 120 mg/kg, 125 mg/kg, 130 mg/kg, 135 mg/kg, 140 mg/kg, 145 mg/kg, 150 mg/kg, 155 mg/kg, 160 mg/kg, 165 mg/kg, 170 mg/kg, 175 mg/kg, 180 mg/kg, 185 mg/kg, 190 mg/kg, 195 mg/kg, 200 mg/kg, and any fraction or integer in between any two of the preceding amounts.

In some embodiments, an nNOS inhibitor composition comprising one or more amino acid-based compounds, may be administered as a suitable dosage form, as described herein, to treat, ameliorate, and/or reduce the severity of M-HNSCC cancer or one or more symptoms thereof in an M-HNSCC cancer subject. In certain embodiments, an nNOS inhibitor composition comprising one or more amino acid-compounds that bind an active site of nNOS selected from the group consisting of Phenylalanine 589, Tryptophan 592, Tyrosine 593, Glutamate 597, Aspartate 602, Arginine 601, Methionine 341, Histidine 342, and Tyrosine711, may be administered as a suitable dosage form, as described herein, to treat, ameliorate, and/or reduce the severity of M-HNSCC cancer or one or more symptoms thereof in an M-HNSCC cancer subject. In some embodiments, an nNOS inhibitor composition comprising one or more amino acid-compounds that bind an active site of nNOS selected from the group consisting of Phenylalanine 589, Tryptophan 592, Tyrosine 593, Glutamate 597, Aspartate 602, Arginine 601, Methionine 341, Histidine 342, and Tyrosine711, may be administered to an M-HNSCC cancer subject to treat, ameliorate, and/or reduce the severity of M-HNSCC cancer or one or more symptoms thereof in the M-HNSCC cancer subject: (i) via a route selected from the group consisting of buccally, inhalation, intraarterially, intraarticularly, intracardially, intracerebroventricularly, intradermally, intramedullary, intramuscularly, intraperitoneally, intrathecally, intravenously, intraventricularly, nasally, ophthalmically, orally, rectally, sublabially, sublingually, subcutaneously, topically, and transdermally; and/or (ii) with a frequency selected from the group consisting of four times daily, three times daily, twice daily, once daily, once every two days, once every three days, once every four days, once every five days, once every six days, once a week, once every eight days, once every nine days, once every ten days, once every two weeks, once every three weeks, and once a month; and/or (iii) at a dosage (based on subject weight) selected from the group consisting of 0.1 μg/kg, 0.5 μg/kg, 1 μg/kg, 1.5 μg/kg, 2.0 μg/kg, 2.5 μg/kg, 3.0 μg/kg, 3.5 μg/kg, 4.0 μg/kg, 4.5 μg/kg, 5.0 μg/kg, 10 μg/kg, 15 μg/kg, 20 μg/kg, 25 μg/kg, 30 μg/kg, 35 μg/kg, 40 μg/kg, 45 μg/kg, 50 μg/kg, 55 μg/kg, 60 μg/kg, 65 μg/kg, 70 μg/kg, 75 μg/kg, 80 μg/kg, 85 μg/kg, 90 μg/kg, 95 μg/kg, 100 μg/kg, 150 μg/kg, 200 μg/kg, 250 μg/kg, 300 μg/kg, 350 μg/kg, 400 μg/kg, 450 μg/kg, 500 μg/kg, 550 μg/kg, 600 μg/kg, 650 μg/kg, 700 μg/kg, 750 μg/kg, 800 μg/kg, 850 μg/kg, 900 μg/kg, 1 mg/kg, 1.5 mg/kg, 2.0 mg/kg, 2.5 mg/kg, 3 mg/kg, 3.5 mg/kg, 4.0 mg/kg, 4.5 mg/kg, 5 mg/kg, 5.5 mg/kg, 6 mg/kg, 6.5 mg/kg, 7 mg/kg, 7.5 mg/kg, 8 mg/kg, 8.5 mg/kg, 9 mg/kg, 9.5 mg/kg, 10 mg/kg 10.5 mg/kg, 11 mg/kg, 11.5 mg/kg, 12 mg/kg, 12.5 mg/kg, 13 mg/kg, 13.5 mg/kg, 14 mg/kg, 14.5 mg/kg, 15 mg/kg, 16 mg/kg, 17 mg/kg, 18 mg/kg, 19 mg/kg, 20 mg/kg, 21 mg/kg, 22 mg/kg, 23 mg/kg, 24 mg/kg, 25 mg/kg, 30 mg/kg, 35 mg/kg, 40 mg/kg, 45 mg/kg, 50 mg/kg, 55 mg/kg, 60 mg/kg, 65 mg/kg, 70 mg/kg, 75 mg/kg, 80 mg/kg, 85 mg/kg, 90 mg/kg, 95 mg/kg, 100 mg/kg, 105 mg/kg, 110 mg/kg, 115 mg/kg, 120 mg/kg, 125 mg/kg, 130 mg/kg, 135 mg/kg, 140 mg/kg, 145 mg/kg, 150 mg/kg, 155 mg/kg, 160 mg/kg, 165 mg/kg, 170 mg/kg, 175 mg/kg, 180 mg/kg, 185 mg/kg, 190 mg/kg, 195 mg/kg, 200 mg/kg, and any fraction or integer in between any two of the preceding amounts.

In some embodiments, a m-HNSCC subject, as described herein, may be a female child or a male child. In certain embodiments, a m-HNSCC subject, as described herein, may be a female adult or a male adult.

In some embodiments, a m-HNSCC subject, as described herein, may have an abnormal expression of one or more antigens compared to an equivalent subject that does not have m-HNSCC. As used herein, the term “equivalent subject,” refers to a subject of the same sex (male/female) and age range (child/adult) of the m-HNSCC subject. For purposes of disclosure, the term “antigen,” when used with respect to abnormal expression, may define one or more cluster of differentiation (“CD”) antigens, such as, for example, CD15, CD44, CD70, CD133, as well as any CD antigen that has an abnormal expression in m-HNSCC that would be readily envisaged by one skilled in the art. In certain embodiments, abnormal expression of an antigen may define an abnormal expression of CD15, an abnormal expression of CD44, an abnormal expression of CD70, an abnormal expression of CD133, an abnormal expression of a CD antigen that has an abnormal expression in m-HNSCC that would be readily envisaged by one skilled in the art, and any combination thereof. In some embodiments, an “abnormal expression,” can refer to a reduced expression of an antigen, as described herein. In certain embodiments, an “abnormal expression,” can refer to an overexpression of an antigen, as described herein. Whether “abnormal expression,” refers to a reduced expression of an antigen or an overexpression of an antigen would be immediately envisaged by the skilled artisan when viewing the term in the context of the particular antigen disclosed and m-HNSCC. For example, in the context of CD44 and m-HNSCC, one skilled in the art would immediately envisage that “abnormal expression” refers to an overexpression of CD44. Treatment, amelioration, and/or reduction in the severity of m-HNSCC or one or more symptoms thereof may be characterized by a restoration to a normal level of expression of one or more antigens from their abnormal state, as described herein. For example, in the context of CD44 and m-HNSCC, treatment, amelioration, and/or reduction in the severity of m-HNSCC or one or more symptoms thereof may be characterized by a reduced expression of CD44.

In certain embodiments, a m-HNSCC subject, as described herein, may have an abnormal expression of one or more proteins compared to an equivalent subject that does not have m-HNSCC. For purposes of disclosure, the term “protein,” when used with respect to abnormal expression, may define one or more proteins and/or enzymes, such as, for example, ALDH1A3, mTOR, p-mTOR, TSC-2, AKT, pAKT, RPS6, p-RPS6, Syp, VEGF, PIGF, as well as any protein or enzyme that has an abnormal expression in m-HNSCC that would be readily envisaged by one skilled in the art. In certain embodiments, abnormal expression of a protein may define an abnormal expression of ALDH1A3, an abnormal expression of mTOR, an abnormal expression of p-mTOR, an abnormal expression of TSC-2, an abnormal expression of AKT, an abnormal expression of pAKT, an abnormal expression of RPS6, an abnormal expression of p-RPS6, an abnormal expression of Syp, an abnormal expression of VEGF, an abnormal expression of PIGF, an abnormal expression of a protein or enzyme that has an abnormal expression in m-HNSCC that would be readily envisaged by one skilled in the art, and any combination thereof. In some embodiments, an “abnormal expression,” can refer to a reduced expression of a protein, as described herein. In certain embodiments, an “abnormal expression,” can refer to an overexpression of a protein, as described herein. Whether “abnormal expression,” refers to a reduced expression of a protein or an overexpression of a protein would be immediately envisaged by the skilled artisan when viewing the term in the context of the particular protein disclosed and m-HNSCC. For example, in the context of m-HNSCC, one skilled in the art would immediately envisage that “abnormal expression” refers to an overexpression of mTOR. Treatment, amelioration, and/or reduction in the severity of m-HNSCC or one or more symptoms thereof may be characterized by a restoration to a normal level of expression of one or more proteins from their abnormal state, as described herein. For example, in the context of m-HNSCC, treatment, amelioration, and/or reduction in the severity of m-HNSCC or one or more symptoms thereof may be characterized by a reduced expression of mTOR.

In certain embodiments, a m-HNSCC subject, as described herein, may have an increased expression of one or more NOS isoforms compared to an equivalent subject that does not have m-HNSCC. For purposes of disclosure, the term “NOS isoform,” refers to nNOS, iNOS, and/or eNOS. In certain embodiments, a m-HNSCC subject, as described herein, may have an increased expression of nNOS compared to an equivalent subject that does not have m-HNSCC. In some embodiments, a m-HNSCC subject, as described herein, may have an increased expression of iNOS compared to an equivalent subject that does not have m-HNSCC. In certain embodiments, a m-HNSCC subject, as described herein, may have an increased expression of eNOS compared to an equivalent subject that does not have m-HNSCC. In some embodiments, a m-HNSCC subject, as described herein, may have an increased expression of nNOS and iNOS compared to an equivalent subject that does not have m-HNSCC. In certain embodiments, a m-HNSCC subject, as described herein, may have an increased expression of nNOS and eNOS compared to an equivalent subject that does not have m-HNSCC. In some embodiments, a m-HNSCC subject, as described herein, may have an increased expression of iNOS and eNOS compared to an equivalent subject that does not have m-HNSCC. In certain embodiments, a m-HNSCC subject, as described herein, may have an increased expression of nNOS, iNOS, and eNOS compared to an equivalent subject that does not have m-HNSCC. Whether a “NOS isoform,” has an increased expression would be immediately envisaged by the skilled artisan when viewing the term in the context of the particular NOS isoform disclosed and m-HNSCC. For example, in the context of m-HNSCC, one skilled in the art would immediately envisage that iNOS and/or eNOS have an increased expression. Treatment, amelioration, and/or reduction in the severity of m-HNSCC or one or more symptoms thereof may be characterized by a reduced expression of one or more NOS isoforms from their increased state, as described herein. For example, in the context of m-HNSCC, treatment, amelioration, and/or reduction in the severity of m-HNSCC or one or more symptoms thereof may be characterized by a reduced expression of iNOS and/or eNOS. For purposes of disclosure, reduction/inhibition of the expression of one or more NOS isoforms may be characterized by a reduction in expression of one or more NOS isoforms by about at least 10%, about at least 20%, about at least 30%, or about at least 40%. In some embodiments, reduction/inhibition of the expression of one or more NOS isoforms may further be characterized by a lack reduction of other, non-implicated NOS isoforms, such as, for example, no more than about 10%, no more than about 5%, no more than about 2%, or no more than about 1% reduction/inhibition of the expression of non-implicated NOS isoforms. As used herein, the term “non-implicated NOS isoforms,” refers to NOS isoforms that do not have an increased expression in m-HNSCC. By way of example, in the context of m-HNSCC, if both iNOS and eNOS have an increased expression, nNOS would define a “non-implicated” NOS isoform. Whether a NOS isoform is a “non-implicated NOS isoform,” would be immediately envisaged by the skilled artisan when viewing the term in the context of the particular NOS isoform disclosed and m-HNSCC.

In some embodiments, a m-HNSCC subject, as described herein, may have an elevated level of one or more NO-indicators compared to an equivalent subject that does not have m-HNSCC. As used herein, the term “NO-indicator,” can refer to 3-nitrotyrosine (3-Ntyr), NADPH-diaphorase, total nitrite concentration, total nitrate concentration, total nitrite and nitrate concentration, as well as any NO-indicator that has an elevated level in m-HNSCC that would be readily envisaged by one skilled in the art. For example, in the context of m-HNSCC, one skilled in the art would immediately envisage that 3-Ntyr can be present at an elevated level. Treatment, amelioration, and/or reduction in the severity of m-HNSCC or one or more symptoms thereof may be characterized by a reduction in the level of one or more NO-indicators. For example, in the context of m-HNSCC, treatment, amelioration, and/or reduction in the severity of m-HNSCC or one or more symptoms thereof may be characterized by a reduction in the level of 3-Ntyr.

In certain embodiments, a NOS inhibitor composition, as described herein, may be administered as a suitable dosage form, as described herein, to treat, ameliorate, and/or reduce the severity of m-HNSCC or one or more symptoms thereof, as an alternative to a conventional therapy for treating, ameliorating, and/or reducing the severity of m-HNSCC or one or more symptoms thereof. For purposes of disclosure, a “conventional therapy,” as described herein, may refer to a treatment that would be recommended by one skilled in the art to treat m-HNSCC, and may define one or more of radiation, chemotherapy, immunotherapy, cell-based therapy, gene therapy, and the like—one skilled in the art would immediately envisage a “conventional therapy” based on the particular m-HNSCC disclosed, as well as characteristics of a m-HNSCC subject, such as, for example, the subject's sex, age, stage of the m-HNSCC, genotype of the m-HNSCC, and the like. Treatment, amelioration, and/or reduction in the severity of m-HNSCC or one or more symptoms thereof may be characterized by an improved outcome compared to a conventional therapy, as described herein. For purposes of the disclosure, an “improved outcome” may define any one of the following: (i) a larger reduction in tumor volume and/or mass compared to a subject administered an equivalent dose of a conventional therapy; (ii) an improvement in the expected rate of survival of a subject, at, for example, 1 month, 3 months, 6 months, 9 months, 1 year, 2 years, 3 years, 5 years, 10 years, or more, compared to the expected rate of survival of a subject administered an equivalent dose of a conventional therapy; (iii) a reduction in the prevalence of side effects resulting from the NOS inhibitor composition compared to a subject administered an equivalent dose of a conventional therapy; (iv) a reduction in the severity of side effects resulting from the NOS inhibitor composition compared to a subject administered an equivalent dose of a conventional therapy; (v) a reduction in the dosage required to achieve an equivalent reduction in tumor volume compared to a subject administered a conventional therapy; (vi) a reduction in the dosage required to achieve an equivalent expected rate of survival of a subject compared to a subject administered a conventional therapy; (vii) a reduction in the likelihood of a recurrence of the m-HNSCC occurring in a subject compared to a subject administered an equivalent dose of a conventional therapy; (viii) a reduction in the likelihood of a metastasis occurring in a subject compared to a subject administered an equivalent dose of a conventional therapy; (ix) a reduction in the time required to be at a medical facility for treatment compared to a subject administered an equivalent dose of a conventional therapy; (x) a reduction in the cost associated with treatment compared to a subject administered an equivalent dose of a conventional therapy; as well as any other improved outcome not expressly recited that would be readily envisaged by one skilled in the art.

In some embodiments, a NOS inhibitor composition, as described herein, may be administered as a suitable dosage form, as described herein, to treat, ameliorate, and/or reduce the severity of m-HNSCC or one or more symptoms thereof, wherein the NOS inhibitor composition comprises a first chemical NOS inhibitor and a second chemical NOS inhibitor. In certain embodiments, the first chemical NOS inhibitor may be administered concurrently with the second chemical NOS inhibitor. In some embodiments, the first chemical NOS inhibitor may be administered prior to the second chemical NOS inhibitor. In certain embodiments, the first chemical NOS inhibitor may be administered subsequently to the second chemical NOS inhibitor. Treatment, amelioration, and/or reduction in the severity of m-HNSCC or one or more symptoms thereof may be characterized by an improved outcome compared an alternative treatment. As used herein, the term “alternative treatment,” may define one or more of a conventional therapy, as described herein, a NOS inhibitor composition comprising a single chemical NOS inhibitor, as described herein, and a NOS inhibitor composition comprising a single biological NOS inhibitor, as described herein. For purposes of the disclosure, an “improved outcome” may define any one of the following: (i) a larger reduction in tumor volume and/or mass compared an alternative treatment; (ii) an improvement in the expected rate of survival of a subject, at, for example, 1 month, 3 months, 6 months, 9 months, 1 year, 2 years, 3 years, 5 years, 10 years, or more, compared to the expected rate of survival of a subject administered an alternative treatment; (iii) a reduction in the prevalence of side effects resulting from the NOS inhibitor composition comprising at least two chemical NOS inhibitors compared to a subject administered an alternative treatment; (iv) a reduction in the severity of side effects resulting from the NOS inhibitor composition comprising at least two chemical NOS inhibitors compared to a subject administered an alternative treatment; (v) a reduction in the dosage required to achieve an equivalent reduction in tumor volume compared to a subject administered an alternative treatment; (vi) a reduction in the dosage required to achieve an equivalent expected rate of survival of a subject compared to a subject administered an alternative treatment; (vii) a reduction in the likelihood of a recurrence of the m-HNSCC in a subject compared to a subject administered an alternative treatment; (viii) a reduction in the likelihood of a metastasis occurring in a subject compared to a subject administered an alternative treatment; (ix) a reduction in the time required to be at a medical facility for treatment compared to a subject administered an alternative treatment; (x) a reduction in the cost associated with treatment compared to a subject administered an alternative treatment; as well as any other improved outcome not expressly recited that would be readily envisaged by one skilled in the art.

In certain embodiments, a NOS inhibitor composition, as described herein, may be used to treat, ameliorate, and/or reduce the severity of m-HNSCC or one or more symptoms thereof, wherein the NOS inhibitor composition comprises at least one chemical NOS inhibitor and at least one biological NOS inhibitor. In some embodiments, the at least one chemical NOS inhibitor may be administered concurrently with the at least one biological NOS inhibitor. In certain embodiments, the at least one chemical NOS inhibitor may be administered prior to the at least one biological NOS inhibitor. In some embodiments, the at least one chemical NOS inhibitor may be administered subsequently to the at least one biological NOS inhibitor. Treatment, amelioration, and/or reduction in the severity of m-HNSCC or one or more symptoms thereof may be characterized by an improved outcome compared an alternative treatment. As used herein, the term “alternative treatment,” may define one or more of a conventional therapy, as described herein, a NOS inhibitor composition comprising a single chemical NOS inhibitor, as described herein, and a NOS inhibitor composition comprising a single biological NOS inhibitor, as described herein. For purposes of the disclosure, an “improved outcome” may define any one of the following: (i) a larger reduction in tumor volume and/or mass compared an alternative treatment; (ii) an improvement in the expected rate of survival of a subject, at, for example, 1 month, 3 months, 6 months, 9 months, 1 year, 2 years, 3 years, 5 years, 10 years, or more, compared to the expected rate of survival of a subject administered an alternative treatment; (iii) a reduction in the prevalence of side effects resulting from the NOS inhibitor composition comprising at least one chemical NOS inhibitor and at least one biological NOS inhibitor compared to a subject administered an alternative treatment; (iv) a reduction in the severity of side effects resulting from the NOS inhibitor composition comprising at least one chemical NOS inhibitor and at least one biological NOS inhibitor compared to a subject administered an alternative treatment; (v) a reduction in the dosage required to achieve an equivalent reduction in tumor volume compared to a subject administered an alternative treatment; (vi) a reduction in the dosage required to achieve an equivalent expected rate of survival of a subject compared to a subject administered an alternative treatment; (vii) a reduction in the likelihood of a recurrence of the m-HNSCC in a subject compared to a subject administered an alternative treatment; (viii) a reduction in the likelihood of a metastasis occurring in a subject compared to a subject administered an alternative treatment; (ix) a reduction in the time required to be at a medical facility for treatment compared to a subject administered an alternative treatment; (x) a reduction in the cost associated with treatment compared to a subject administered an alternative treatment; as well as any other improved outcome not expressly recited that would be readily envisaged by one skilled in the art.

In some embodiments, a NOS inhibitor composition, as described herein, may be combined with a conventional therapy, as described herein, to treat, ameliorate, and/or reduce the severity of m-HNSCC or one or more symptoms thereof. In certain embodiments, the NOS inhibitor composition may be administered concurrently with the conventional therapy. In some embodiments, the NOS inhibitor composition may be administered prior to the conventional therapy. In certain embodiments, the NOS inhibitor composition may be administered subsequently to the conventional therapy. Treatment, amelioration, and/or reduction in the severity of m-HNSCC or one or more symptoms thereof may be characterized by an improved outcome compared an alternative treatment. As used herein, the term “alternative treatment,” may define one or more of a conventional therapy, as described herein, a NOS inhibitor composition comprising a single chemical NOS inhibitor, as described herein, and a NOS inhibitor composition comprising a single biological NOS inhibitor, as described herein. For purposes of the disclosure, an “improved outcome” may define any one of the following: (i) a larger reduction in tumor volume and/or mass compared an alternative treatment; (ii) an improvement in the expected rate of survival of a subject, at, for example, 1 month, 3 months, 6 months, 9 months, 1 year, 2 years, 3 years, 5 years, 10 years, or more, compared to the expected rate of survival of a subject administered an alternative treatment; (iii) a reduction in the prevalence of side effects resulting from the NOS inhibitor composition and conventional therapy compared to a subject administered an alternative treatment; (iv) a reduction in the severity of side effects resulting from the NOS inhibitor composition and conventional therapy compared to a subject administered an alternative treatment; (v) a reduction in the dosage required to achieve an equivalent reduction in tumor volume compared to a subject administered an alternative treatment; (vi) a reduction in the dosage required to achieve an equivalent expected rate of survival of a subject compared to a subject administered an alternative treatment; (vii) a reduction in the likelihood of a recurrence of the m-HNSCC in a subject compared to a subject administered an alternative treatment; (viii) a reduction in the likelihood of a metastasis occurring in a subject compared to a subject administered an alternative treatment; (ix) a reduction in the time required to be at a medical facility for treatment compared to a subject administered an alternative treatment; (x) a reduction in the cost associated with treatment compared to a subject administered an alternative treatment; as well as any other improved outcome not expressly recited that would be readily envisaged by one skilled in the art.

In some embodiments, a “conventional therapy,” as that term is used with respect to combined administration with a NOS inhibitor composition, may refer to a conventional chemotherapy. For purposes of the disclosure, a “conventional chemotherapy,” as described herein may be a compound selected from the group consisting of abemaciclib, abiraterone acetate, acalabrutinib, adagrasib, afatinib dimaleate, albumin-bound paclitaxel, albumin-bound rapamycin (Sirolimus Protein-Bound Particles), alectinib, alpelisib, amifostine, aminolevulinic acid hydrochloride, anastrozole, apalutamide, aprepitant, arsenic trioxide, asciminib hydrochloride, avapritinib, axitinib, azacytidine, belinostat, belzutifan, bendamustine hydrochloride, bexarotene, bicalutamide, binimetinib, bleomycin, bleomycin sulfate, bortezomib, bosutinib, brigatinib, busulfan, cabazitaxel, cabozantinib-S-malate, capecitabine, capmatinib hydrochloride, carboplatin, carfilzomib, carmustine, cedazuridine, ceritinib, chlorambucil, cisplatin, cladribine, clofarabine, cobimetinib fumarate, copanlisib hydrochloride, crizotinib, cyclophosphamide, cytarabine, dabrafenib mesylate, dacarbazine, dacomitinib, dactinomycin, darolutamide, dasatinib, daunorubicin hydrochloride, decitabine, defibrotide sodium, degarelix, dexamethasone, dexrazoxane hydrochloride, docetaxel, doxorubicin hydrochloride, duvelisib, elacestrant dihydrochloride, eltrombopag olamine, enasidenib mesylate, encorafenib, entrectinib, enzalutamide, epirubicin hydrochloride, erdafitinib, eribulin mesylate, erlotinib hydrochloride, etoposide, etoposide phosphate, everolimus, exemestane, fedratinib hydrochloride, fludarabine phosphate, fluorouracil, flutamide, folinic acid, fostamatinib disodium, fulvestrant, futibatinib, gefitinib, gemcitabine hydrochloride, gilteritinib fumarate, glasdegib maleate, goserelin acetate, granisetron, granisetron hydrochloride, hydroxyurea, ibrutinib, idarubicin hydrochloride, idelalisib, ifosfamide, imatinib mesylate, imiquimod, infigratinib phosphate, iobenguane I-131, irinotecan hydrochloride, ivosidenib, ixabepilone, ixazomib citrate, lanreotide acetate, lapatinib ditosylate, larotrectinib sulfate, lenalidomide, lenvatinib mesylate, letrozole, leucovorin calcium, leuprolide acetate, liposomal cytarabine, liposomal doxorubicin hydrochloride, liposomal irinotecan hydrochloride, lomustine, lorlatinib, lurbinectedin, Lu-177 dotatate, Lu-177 vipivotide tetraxetan, mechlorethamine hydrochloride, megestrol acetate, melphalan, melphalan hydrochloride, mercaptopurine, mesna, methotrexate, methotrexate sodium, methylnaltrexone bromide, midostaurin, mitomycin, mitoxantrone hydrochloride, mobocertinib succinate, nelarabine, neratinib maleate, netupitant hydrochloride, nilutamide, nilotinib, niraparib tosylate monohydrate, olaparib, olutasidenib, omacetaxine mepesuccinate, ondansetron hydrochloride, osimertinib mesylate, oxaliplatin, paclitaxel, pacritinib citrate, palbociclib, palonosetron hydrochloride, pamidronate disodium, pazopanib hydrochloride, pemetrexed disodium, pemigatinib, pexidartinib hydrochloride, pirtobrutinib, plerixafor, pomalidomide, ponatinib hydrochloride, pralatrexate, pralsetinib, prednisone, procarbazine hydrochloride, propranolol hydrochloride, Ra-223 dichloride, raloxifene hydrochloride, regorafenib, relugolix, ribociclib, ripretinib, rolapitant hydrochloride, romidepsin, rucaparib camsylate, ruxolitinib phosphate, selinexor, selpercatinib, selumetinib sulfate, sonidegib, sorafenib tosylate, sotorasib, sunitinib malate, talazoparib tosylate, talc, tamoxifen citrate, tazemetostat hydrobromide, temozolomide, temsirolimus, tepotinib hydrochloride, thalidomide, thioguanine, thiotepa, tipiracil hydrochloride, tivozanib hydrochloride, topotecan hydrochloride, toremifene, trabectedin, trametinib dimethyl sulfoxide, tretinoin, trifluridine hydrochloride, tucatinib, uridine triacetate, valrubicin, vandetanib, vemurafenib, venetoclax, vinblastine sulfate, vincristine sulfate, vinorelbine tartrate, vismodegib, vorinostat, zanubrutinib, zoledronic acid, and any combination thereof. One skilled in the art would immediately envisage an appropriate “conventional chemotherapy” based on the particular m-HNSCC disclosed, as well as characteristics of a m-HNSCC subject, such as, for example, the subject's sex, age, stage of the m-HNSCC, genotype of the m-HNSCC, and the like.

In certain embodiments, a “conventional therapy,” as that term is used with respect to combined administration with a NOS inhibitor composition, may refer to a conventional immunotherapy. For purposes of the disclosure, a “conventional immunotherapy,” as described herein may be an immunotherapy, cell-based therapy, and/or gene-based therapy selected from the group consisting of ado-trastuzumab emtansine, aldesleukin, alemtuzumab, amivantamab-vmjw, asparaginase Erwinia chrysanthemi, asparaginase Erwinia chrysanthemi (recombinant)-rywn, atezolizumab, avelumab, axicabtagene ciloleucel, bevacizumab, blinatumomab, brentuximab vedotin, brexucabtagene autoleucel, calaspargase pegol-mknl, camrelizumab, caplacizumab-yhdp, cemiplimab-rwlc, cetuximab, ciltacabtagene autoleucel, daratumumab, daratumumab and hyaluronidase-fihj, darbepoetin alfa, denileukin diftitox, denosumab, dinutuximab, dostarlimab-gxly, durvalumab, elotuzumab, emapalumab-lzsg, enfortumab vedotin-ejfv, epoetin alfa, fam-trastuzumab deruxtecan-nxki, filgrastim, gemtuzumab ozogamicin, glucarpidase, ibritumomab tiuxetan, idecabtagene vicleucel, inotuzumab ozogamicin, ipilimumab, isatuximab-irfc, lisocabtagene maraleucel, loncastuximab tesirine-lpyl, luspatercept-aamt, margetuximab-cmkb, mirvetuximab soravtansine-gynx, mogamulizumab-kpkc, mosunetuzumab-axgb, moxetumomab pasudotox-tdfk, nadofaragene firadenovec-vncg, naxitamab-gqgk, necitumumab, nivolumab, nivolumab and relatlimab-rmbw, obinutuzumab, ofatumumab, palifermin, panitumumab, pegaspargase, pegfilgrastim, pembrolizumab, pertuzumab, pertuzumab, trastuzumab, and hyaluronidase-zzxf, polatuzumab vedotin-piiq, ramucirumab, rasburicase, ravulizumab-cwvz, recombinant HPV bivalent vaccine, recombinant HPV nonavalent vaccine, recombinant HPV quadrivalent vaccine, recombinant interferon alfa-2b, rituximab, rituximab and hyaluronidase human, romiplostim, ropeginterferon alfa-2b-njft, sacituzumab govitecan-hziy, siltuximab, sintilimab, sipuleucel-T, tafasitamab-cxix, tagraxofusp-erzs, talimogene laherparepvec, tebentafusp-tebn, teclistamab-cqyv, tisagenlecleucel, tislelizumab, tisotumab vedotin-tftv, tocilizumab, toripalimab, trastuzumab, trastuzumab and hyaluronidase-oysk, tremelimumab-actl, ziv-aflibercept, and any combination thereof. One skilled in the art would immediately envisage an appropriate “conventional immunotherapy” based on the particular m-HNSCC disclosed, as well as characteristics of a m-HNSCC subject, such as, for example, the subject's sex, age, stage of the m-HNSCC, genotype of the m-HNSCC, and the like.

In certain embodiments, a NOS inhibitor composition, as described herein, may be administered as a suitable dosage form, as described herein, prior to, or alternatively, following a surgery to remove or resect m-HNSCC.

In some embodiments, a NOS inhibitor composition, as described herein, may be administered as a suitable dosage form, as described herein, to restore and/or maintain healthy and/or optimal expression levels of one or more antigens, as described herein, such as, for example, CD15, CD44, CD70, CD133, as well as any CD antigen that has an abnormal expression in m-HNSCC that would be readily envisaged by one skilled in the art, in a subject. In certain embodiments, the subject may have m-HNSCC or one or more symptoms thereof. As used herein, the terms “healthy” and “optimal” refer to expression levels of one or more antigens that would be typical of an equivalent subject that does not have m-HNSCC.

In certain embodiments, a NOS inhibitor composition, as described herein, may be administered as a suitable dosage form, as described herein, to restore and/or maintain healthy and/or optimal expression levels of one or more proteins, as described herein, such as, for example, ALDH1A3, mTOR, p-mTOR, TSC-2, AKT, pAKT, RPS6, p-RPS6, Syp, VEGF, PIGF, as well as any protein or enzyme that has an abnormal expression in m-HNSCC that would be readily envisaged by one skilled in the art, in a subject. In certain embodiments, the subject may have m-HNSCC or one or more symptoms thereof. As used herein, the terms “healthy” and “optimal” refer to expression levels of one or more proteins that would be typical of an equivalent subject that does not have m-HNSCC.

In some embodiments, a NOS inhibitor composition, as described herein, may be administered as a suitable dosage form, as described herein, to restore and/or maintain healthy and/or optimal expression levels of one or more NOS isoforms, as described herein, such as, for example, nNOS, iNOS, and eNOS, in a subject. In certain embodiments, the subject may have m-HNSCC or one or more symptoms thereof. As used herein, the terms “healthy” and “optimal” refer to expression levels of one or more NOS isoforms that would be typical of an equivalent subject that does not have m-HNSCC.

In certain embodiments, a NOS inhibitor composition, as described herein, may be administered as a suitable dosage form, as described herein, to restore and/or maintain healthy and/or optimal levels of one or more NO-indicators, as described herein, such as, for example, 3-Ntyr, NADPH-diaphorase, total nitrite concentration, total nitrate concentration, total nitrite and nitrate concentration, as well as any NO-indicator that has an elevated level in m-HNSCC that would be readily envisaged by one skilled in the art, in a subject. In certain embodiments, the subject may have m-HNSCC or one or more symptoms thereof. As used herein, the terms “healthy” and “optimal” refer to levels of one or more NO-indicators that would be typical of an equivalent subject that does not have m-HNSCC.

In certain embodiments, a NOS inhibitor composition, as described herein, may be administered as a suitable dosage form, as described herein, to treat, ameliorate, and/or reduce the severity of squamous cell carcinoma of the skin (“s-SCC”) or one or more symptoms thereof in a subject (“s-SCC subject”).

In some embodiments, a NOS inhibitor composition comprising 7-NI and/or a salt thereof may be administered as a suitable dosage form, as described herein, to treat, ameliorate, and/or reduce the severity of S-SCC cancer or one or more symptoms thereof in an S-SCC cancer subject. In certain embodiments, an nNOS inhibitor composition comprising 7-NI and/or a salt thereof may be administered to an S-SCC cancer subject to treat, ameliorate, and/or reduce the severity of S-SCC cancer or one or more symptoms thereof in the S-SCC cancer subject: (i) via a route selected from the group consisting of buccally, inhalation, intraarterially, intraarticularly, intracardially, intracerebroventricularly, intradermally, intramedullary, intramuscularly, intraperitoneally, intrathecally, intravenously, intraventricularly, nasally, ophthalmically, orally, rectally, sublabially, sublingually, subcutaneously, topically, and transdermally; and/or (ii) with a frequency selected from the group consisting of four times daily, three times daily, twice daily, once daily, once every two days, once every three days, once every four days, once every five days, once every six days, once a week, once every eight days, once every nine days, once every ten days, once every two weeks, once every three weeks, and once a month; and/or (iii) at a dosage (based on subject weight) selected from the group consisting of 0.1 μg/kg, 0.5 μg/kg, 1 μg/kg, 1.5 μg/kg, 2.0 μg/kg, 2.5 μg/kg, 3.0 μg/kg, 3.5 μg/kg, 4.0 μg/kg, 4.5 μg/kg, 5.0 μg/kg, 10 μg/kg, 15 μg/kg, 20 μg/kg, 25 μg/kg, 30 μg/kg, 35 μg/kg, 40 μg/kg, 45 μg/kg, 50 μg/kg, 55 μg/kg, 60 μg/kg, 65 μg/kg, 70 μg/kg, 75 μg/kg, 80 μg/kg, 85 μg/kg, 90 μg/kg, 95 μg/kg, 100 μg/kg, 150 μg/kg, 200 μg/kg, 250 μg/kg, 300 μg/kg, 350 μg/kg, 400 μg/kg, 450 μg/kg, 500 μg/kg, 550 μg/kg, 600 μg/kg, 650 μg/kg, 700 μg/kg, 750 μg/kg, 800 μg/kg, 850 μg/kg, 900 μg/kg, 1 mg/kg, 1.5 mg/kg, 2.0 mg/kg, 2.5 mg/kg, 3 mg/kg, 3.5 mg/kg, 4.0 mg/kg, 4.5 mg/kg, 5 mg/kg, 5.5 mg/kg, 6 mg/kg, 6.5 mg/kg, 7 mg/kg, 7.5 mg/kg, 8 mg/kg, 8.5 mg/kg, 9 mg/kg, 9.5 mg/kg, 10 mg/kg 10.5 mg/kg, 11 mg/kg, 11.5 mg/kg, 12 mg/kg, 12.5 mg/kg, 13 mg/kg, 13.5 mg/kg, 14 mg/kg, 14.5 mg/kg, 15 mg/kg, 16 mg/kg, 17 mg/kg, 18 mg/kg, 19 mg/kg, 20 mg/kg, 21 mg/kg, 22 mg/kg, 23 mg/kg, 24 mg/kg, 25 mg/kg, 30 mg/kg, 35 mg/kg, 40 mg/kg, 45 mg/kg, 50 mg/kg, 55 mg/kg, 60 mg/kg, 65 mg/kg, 70 mg/kg, 75 mg/kg, 80 mg/kg, 85 mg/kg, 90 mg/kg, 95 mg/kg, 100 mg/kg, 105 mg/kg, 110 mg/kg, 115 mg/kg, 120 mg/kg, 125 mg/kg, 130 mg/kg, 135 mg/kg, 140 mg/kg, 145 mg/kg, 150 mg/kg, 155 mg/kg, 160 mg/kg, 165 mg/kg, 170 mg/kg, 175 mg/kg, 180 mg/kg, 185 mg/kg, 190 mg/kg, 195 mg/kg, 200 mg/kg, and any fraction or integer in between any two of the preceding amounts.

In certain embodiments, a NOS inhibitor composition comprising 3-Br-7-NI and/or a salt thereof may be administered as a suitable dosage form, as described herein, to treat, ameliorate, and/or reduce the severity of S-SCC cancer or one or more symptoms thereof in an S-SCC cancer subject. In some embodiments, an nNOS inhibitor composition comprising 3-Br-7-NI and/or a salt thereof may be administered to an S-SCC cancer subject to treat, ameliorate, and/or reduce the severity of S-SCC cancer or one or more symptoms thereof in the S-SCC cancer subject: (i) via a route selected from the group consisting of buccally, inhalation, intraarterially, intraarticularly, intracardially, intracerebroventricularly, intradermally, intramedullary, intramuscularly, intraperitoneally, intrathecally, intravenously, intraventricularly, nasally, ophthalmically, orally, rectally, sublabially, sublingually, subcutaneously, topically, and transdermally; and/or (ii) with a frequency selected from the group consisting of four times daily, three times daily, twice daily, once daily, once every two days, once every three days, once every four days, once every five days, once every six days, once a week, once every eight days, once every nine days, once every ten days, once every two weeks, once every three weeks, and once a month; and/or (iii) at a dosage (based on subject weight) selected from the group consisting of 0.1 μg/kg, 0.5 μg/kg, 1 μg/kg, 1.5 μg/kg, 2.0 μg/kg, 2.5 μg/kg, 3.0 μg/kg, 3.5 μg/kg, 4.0 μg/kg, 4.5 μg/kg, 5.0 μg/kg, 10 μg/kg, 15 μg/kg, 20 μg/kg, 25 μg/kg, 30 μg/kg, 35 μg/kg, 40 μg/kg, 45 μg/kg, 50 μg/kg, 55 μg/kg, 60 μg/kg, 65 μg/kg, 70 μg/kg, 75 μg/kg, 80 μg/kg, 85 μg/kg, 90 μg/kg, 95 μg/kg, 100 μg/kg, 150 μg/kg, 200 μg/kg, 250 μg/kg, 300 μg/kg, 350 μg/kg, 400 μg/kg, 450 μg/kg, 500 μg/kg, 550 μg/kg, 600 μg/kg, 650 μg/kg, 700 μg/kg, 750 μg/kg, 800 μg/kg, 850 μg/kg, 900 μg/kg, 1 mg/kg, 1.5 mg/kg, 2.0 mg/kg, 2.5 mg/kg, 3 mg/kg, 3.5 mg/kg, 4.0 mg/kg, 4.5 mg/kg, 5 mg/kg, 5.5 mg/kg, 6 mg/kg, 6.5 mg/kg, 7 mg/kg, 7.5 mg/kg, 8 mg/kg, 8.5 mg/kg, 9 mg/kg, 9.5 mg/kg, 10 mg/kg 10.5 mg/kg, 11 mg/kg, 11.5 mg/kg, 12 mg/kg, 12.5 mg/kg, 13 mg/kg, 13.5 mg/kg, 14 mg/kg, 14.5 mg/kg, 15 mg/kg, 16 mg/kg, 17 mg/kg, 18 mg/kg, 19 mg/kg, 20 mg/kg, 21 mg/kg, 22 mg/kg, 23 mg/kg, 24 mg/kg, 25 mg/kg, 30 mg/kg, 35 mg/kg, 40 mg/kg, 45 mg/kg, 50 mg/kg, 55 mg/kg, 60 mg/kg, 65 mg/kg, 70 mg/kg, 75 mg/kg, 80 mg/kg, 85 mg/kg, 90 mg/kg, 95 mg/kg, 100 mg/kg, 105 mg/kg, 110 mg/kg, 115 mg/kg, 120 mg/kg, 125 mg/kg, 130 mg/kg, 135 mg/kg, 140 mg/kg, 145 mg/kg, 150 mg/kg, 155 mg/kg, 160 mg/kg, 165 mg/kg, 170 mg/kg, 175 mg/kg, 180 mg/kg, 185 mg/kg, 190 mg/kg, 195 mg/kg, 200 mg/kg, and any fraction or integer in between any two of the preceding amounts.

In some embodiments, a NOS inhibitor composition comprising L-NPA and/or a salt thereof, such as, for example, L-NPA hydrochloride, may be administered as a suitable dosage form, as described herein, to treat, ameliorate, and/or reduce the severity of S-SCC cancer or one or more symptoms thereof in an S-SCC cancer subject. In certain embodiments, an nNOS inhibitor composition comprising L-NPA and/or a salt thereof, such as, for example, L-NPA hydrochloride may be administered to an S-SCC cancer subject to treat, ameliorate, and/or reduce the severity of S-SCC cancer or one or more symptoms thereof in the S-SCC cancer subject: (i) via a route selected from the group consisting of buccally, inhalation, intraarterially, intraarticularly, intracardially, intracerebroventricularly, intradermally, intramedullary, intramuscularly, intraperitoneally, intrathecally, intravenously, intraventricularly, nasally, ophthalmically, orally, rectally, sublabially, sublingually, subcutaneously, topically, and transdermally; and/or (ii) with a frequency selected from the group consisting of four times daily, three times daily, twice daily, once daily, once every two days, once every three days, once every four days, once every five days, once every six days, once a week, once every eight days, once every nine days, once every ten days, once every two weeks, once every three weeks, and once a month; and/or (iii) at a dosage (based on subject weight) selected from the group consisting of 0.1 μg/kg, 0.5 μg/kg, 1 μg/kg, 1.5 μg/kg, 2.0 μg/kg, 2.5 μg/kg, 3.0 μg/kg, 3.5 μg/kg, 4.0 μg/kg, 4.5 μg/kg, 5.0 μg/kg, 10 μg/kg, 15 μg/kg, 20 μg/kg, 25 μg/kg, 30 μg/kg, 35 μg/kg, 40 μg/kg, 45 μg/kg, 50 μg/kg, 55 μg/kg, 60 μg/kg, 65 μg/kg, 70 μg/kg, 75 μg/kg, 80 μg/kg, 85 μg/kg, 90 μg/kg, 95 μg/kg, 100 μg/kg, 150 μg/kg, 200 μg/kg, 250 μg/kg, 300 μg/kg, 350 μg/kg, 400 μg/kg, 450 μg/kg, 500 μg/kg, 550 μg/kg, 600 μg/kg, 650 μg/kg, 700 μg/kg, 750 μg/kg, 800 μg/kg, 850 μg/kg, 900 μg/kg, 1 mg/kg, 1.5 mg/kg, 2.0 mg/kg, 2.5 mg/kg, 3 mg/kg, 3.5 mg/kg, 4.0 mg/kg, 4.5 mg/kg, 5 mg/kg, 5.5 mg/kg, 6 mg/kg, 6.5 mg/kg, 7 mg/kg, 7.5 mg/kg, 8 mg/kg, 8.5 mg/kg, 9 mg/kg, 9.5 mg/kg, 10 mg/kg 10.5 mg/kg, 11 mg/kg, 11.5 mg/kg, 12 mg/kg, 12.5 mg/kg, 13 mg/kg, 13.5 mg/kg, 14 mg/kg, 14.5 mg/kg, 15 mg/kg, 16 mg/kg, 17 mg/kg, 18 mg/kg, 19 mg/kg, 20 mg/kg, 21 mg/kg, 22 mg/kg, 23 mg/kg, 24 mg/kg, 25 mg/kg, 30 mg/kg, 35 mg/kg, 40 mg/kg, 45 mg/kg, 50 mg/kg, 55 mg/kg, 60 mg/kg, 65 mg/kg, 70 mg/kg, 75 mg/kg, 80 mg/kg, 85 mg/kg, 90 mg/kg, 95 mg/kg, 100 mg/kg, 105 mg/kg, 110 mg/kg, 115 mg/kg, 120 mg/kg, 125 mg/kg, 130 mg/kg, 135 mg/kg, 140 mg/kg, 145 mg/kg, 150 mg/kg, 155 mg/kg, 160 mg/kg, 165 mg/kg, 170 mg/kg, 175 mg/kg, 180 mg/kg, 185 mg/kg, 190 mg/kg, 195 mg/kg, 200 mg/kg, and any fraction or integer in between any two of the preceding amounts.

In certain embodiments, a NOS inhibitor composition comprising L-NIO and/or a salt thereof, such as, for example, L-NIO dihydrochloride, may be administered as a suitable dosage form, as described herein, to treat, ameliorate, and/or reduce the severity of S-SCC cancer or one or more symptoms thereof in an S-SCC cancer subject. In some embodiments, an nNOS inhibitor composition comprising L-NIO and/or a salt thereof, such as, for example, L-NIO dihydrochloride, may be administered to an S-SCC cancer subject to treat, ameliorate, and/or reduce the severity of S-SCC cancer or one or more symptoms thereof in the S-SCC cancer subject: (i) via a route selected from the group consisting of buccally, inhalation, intraarterially, intraarticularly, intracardially, intracerebroventricularly, intradermally, intramedullary, intramuscularly, intraperitoneally, intrathecally, intravenously, intraventricularly, nasally, ophthalmically, orally, rectally, sublabially, sublingually, subcutaneously, topically, and transdermally; and/or (ii) with a frequency selected from the group consisting of four times daily, three times daily, twice daily, once daily, once every two days, once every three days, once every four days, once every five days, once every six days, once a week, once every eight days, once every nine days, once every ten days, once every two weeks, once every three weeks, and once a month; and/or (iii) at a dosage (based on subject weight) selected from the group consisting of 0.1 μg/kg, 0.5 μg/kg, 1 μg/kg, 1.5 μg/kg, 2.0 μg/kg, 2.5 μg/kg, 3.0 μg/kg, 3.5 μg/kg, 4.0 μg/kg, 4.5 μg/kg, 5.0 μg/kg, 10 μg/kg, 15 μg/kg, 20 μg/kg, 25 μg/kg, 30 μg/kg, 35 μg/kg, 40 μg/kg, 45 μg/kg, 50 μg/kg, 55 μg/kg, 60 μg/kg, 65 μg/kg, 70 μg/kg, 75 μg/kg, 80 μg/kg, 85 μg/kg, 90 μg/kg, 95 μg/kg, 100 μg/kg, 150 μg/kg, 200 μg/kg, 250 μg/kg, 300 μg/kg, 350 μg/kg, 400 μg/kg, 450 μg/kg, 500 μg/kg, 550 μg/kg, 600 μg/kg, 650 μg/kg, 700 μg/kg, 750 μg/kg, 800 μg/kg, 850 μg/kg, 900 μg/kg, 1 mg/kg, 1.5 mg/kg, 2.0 mg/kg, 2.5 mg/kg, 3 mg/kg, 3.5 mg/kg, 4.0 mg/kg, 4.5 mg/kg, 5 mg/kg, 5.5 mg/kg, 6 mg/kg, 6.5 mg/kg, 7 mg/kg, 7.5 mg/kg, 8 mg/kg, 8.5 mg/kg, 9 mg/kg, 9.5 mg/kg, 10 mg/kg 10.5 mg/kg, 11 mg/kg, 11.5 mg/kg, 12 mg/kg, 12.5 mg/kg, 13 mg/kg, 13.5 mg/kg, 14 mg/kg, 14.5 mg/kg, 15 mg/kg, 16 mg/kg, 17 mg/kg, 18 mg/kg, 19 mg/kg, 20 mg/kg, 21 mg/kg, 22 mg/kg, 23 mg/kg, 24 mg/kg, 25 mg/kg, 30 mg/kg, 35 mg/kg, 40 mg/kg, 45 mg/kg, 50 mg/kg, 55 mg/kg, 60 mg/kg, 65 mg/kg, 70 mg/kg, 75 mg/kg, 80 mg/kg, 85 mg/kg, 90 mg/kg, 95 mg/kg, 100 mg/kg, 105 mg/kg, 110 mg/kg, 115 mg/kg, 120 mg/kg, 125 mg/kg, 130 mg/kg, 135 mg/kg, 140 mg/kg, 145 mg/kg, 150 mg/kg, 155 mg/kg, 160 mg/kg, 165 mg/kg, 170 mg/kg, 175 mg/kg, 180 mg/kg, 185 mg/kg, 190 mg/kg, 195 mg/kg, 200 mg/kg, and any fraction or integer in between any two of the preceding amounts.

In some embodiments, a NOS inhibitor composition comprising ARL-17477 and/or a salt thereof, such as, for example, ARL-17477 dihydrochloride, may be administered as a suitable dosage form, as described herein, to treat, ameliorate, and/or reduce the severity of S-SCC cancer or one or more symptoms thereof in an S-SCC cancer subject. In certain embodiments, an nNOS inhibitor composition comprising ARL-17477 and/or a salt thereof, such as, for example, ARL-17477 dihydrochloride, may be administered to an S-SCC cancer subject to treat, ameliorate, and/or reduce the severity of S-SCC cancer or one or more symptoms thereof in the S-SCC cancer subject: (i) via a route selected from the group consisting of buccally, inhalation, intraarterially, intraarticularly, intracardially, intracerebroventricularly, intradermally, intramedullary, intramuscularly, intraperitoneally, intrathecally, intravenously, intraventricularly, nasally, ophthalmically, orally, rectally, sublabially, sublingually, subcutaneously, topically, and transdermally; and/or (ii) with a frequency selected from the group consisting of four times daily, three times daily, twice daily, once daily, once every two days, once every three days, once every four days, once every five days, once every six days, once a week, once every eight days, once every nine days, once every ten days, once every two weeks, once every three weeks, and once a month; and/or (iii) at a dosage (based on subject weight) selected from the group consisting of 0.1 μg/kg, 0.5 μg/kg, 1 μg/kg, 1.5 μg/kg, 2.0 μg/kg, 2.5 μg/kg, 3.0 μg/kg, 3.5 μg/kg, 4.0 μg/kg, 4.5 μg/kg, 5.0 μg/kg, 10 μg/kg, 15 μg/kg, 20 μg/kg, 25 μg/kg, 30 μg/kg, 35 μg/kg, 40 μg/kg, 45 μg/kg, 50 μg/kg, 55 μg/kg, 60 μg/kg, 65 μg/kg, 70 μg/kg, 75 μg/kg, 80 μg/kg, 85 μg/kg, 90 μg/kg, 95 μg/kg, 100 μg/kg, 150 μg/kg, 200 μg/kg, 250 μg/kg, 300 μg/kg, 350 μg/kg, 400 μg/kg, 450 μg/kg, 500 μg/kg, 550 μg/kg, 600 μg/kg, 650 μg/kg, 700 μg/kg, 750 μg/kg, 800 μg/kg, 850 μg/kg, 900 μg/kg, 1 mg/kg, 1.5 mg/kg, 2.0 mg/kg, 2.5 mg/kg, 3 mg/kg, 3.5 mg/kg, 4.0 mg/kg, 4.5 mg/kg, 5 mg/kg, 5.5 mg/kg, 6 mg/kg, 6.5 mg/kg, 7 mg/kg, 7.5 mg/kg, 8 mg/kg, 8.5 mg/kg, 9 mg/kg, 9.5 mg/kg, 10 mg/kg 10.5 mg/kg, 11 mg/kg, 11.5 mg/kg, 12 mg/kg, 12.5 mg/kg, 13 mg/kg, 13.5 mg/kg, 14 mg/kg, 14.5 mg/kg, 15 mg/kg, 16 mg/kg, 17 mg/kg, 18 mg/kg, 19 mg/kg, 20 mg/kg, 21 mg/kg, 22 mg/kg, 23 mg/kg, 24 mg/kg, 25 mg/kg, 30 mg/kg, 35 mg/kg, 40 mg/kg, 45 mg/kg, 50 mg/kg, 55 mg/kg, 60 mg/kg, 65 mg/kg, 70 mg/kg, 75 mg/kg, 80 mg/kg, 85 mg/kg, 90 mg/kg, 95 mg/kg, 100 mg/kg, 105 mg/kg, 110 mg/kg, 115 mg/kg, 120 mg/kg, 125 mg/kg, 130 mg/kg, 135 mg/kg, 140 mg/kg, 145 mg/kg, 150 mg/kg, 155 mg/kg, 160 mg/kg, 165 mg/kg, 170 mg/kg, 175 mg/kg, 180 mg/kg, 185 mg/kg, 190 mg/kg, 195 mg/kg, 200 mg/kg, and any fraction or integer in between any two of the preceding amounts.

In certain embodiments, a NOS inhibitor composition comprising L-NNA and/or a salt thereof may be administered as a suitable dosage form, as described herein, to treat, ameliorate, and/or reduce the severity of S-SCC cancer or one or more symptoms thereof in an S-SCC cancer subject. In some embodiments, an nNOS inhibitor composition comprising L-NNA and/or a salt thereof may be administered to an S-SCC cancer subject to treat, ameliorate, and/or reduce the severity of S-SCC cancer or one or more symptoms thereof in the S-SCC cancer subject: (i) via a route selected from the group consisting of buccally, inhalation, intraarterially, intraarticularly, intracardially, intracerebroventricularly, intradermally, intramedullary, intramuscularly, intraperitoneally, intrathecally, intravenously, intraventricularly, nasally, ophthalmically, orally, rectally, sublabially, sublingually, subcutaneously, topically, and transdermally; and/or (ii) with a frequency selected from the group consisting of four times daily, three times daily, twice daily, once daily, once every two days, once every three days, once every four days, once every five days, once every six days, once a week, once every eight days, once every nine days, once every ten days, once every two weeks, once every three weeks, and once a month; and/or (iii) at a dosage (based on subject weight) selected from the group consisting of 0.1 μg/kg, 0.5 μg/kg, 1 μg/kg, 1.5 μg/kg, 2.0 μg/kg, 2.5 μg/kg, 3.0 μg/kg, 3.5 μg/kg, 4.0 μg/kg, 4.5 μg/kg, 5.0 μg/kg, 10 μg/kg, 15 μg/kg, 20 μg/kg, 25 μg/kg, 30 μg/kg, 35 μg/kg, 40 μg/kg, 45 μg/kg, 50 μg/kg, 55 μg/kg, 60 μg/kg, 65 μg/kg, 70 μg/kg, 75 μg/kg, 80 μg/kg, 85 μg/kg, 90 μg/kg, 95 μg/kg, 100 μg/kg, 150 μg/kg, 200 μg/kg, 250 μg/kg, 300 μg/kg, 350 μg/kg, 400 μg/kg, 450 μg/kg, 500 μg/kg, 550 μg/kg, 600 μg/kg, 650 μg/kg, 700 μg/kg, 750 μg/kg, 800 μg/kg, 850 μg/kg, 900 μg/kg, 1 mg/kg, 1.5 mg/kg, 2.0 mg/kg, 2.5 mg/kg, 3 mg/kg, 3.5 mg/kg, 4.0 mg/kg, 4.5 mg/kg, 5 mg/kg, 5.5 mg/kg, 6 mg/kg, 6.5 mg/kg, 7 mg/kg, 7.5 mg/kg, 8 mg/kg, 8.5 mg/kg, 9 mg/kg, 9.5 mg/kg, 10 mg/kg 10.5 mg/kg, 11 mg/kg, 11.5 mg/kg, 12 mg/kg, 12.5 mg/kg, 13 mg/kg, 13.5 mg/kg, 14 mg/kg, 14.5 mg/kg, 15 mg/kg, 16 mg/kg, 17 mg/kg, 18 mg/kg, 19 mg/kg, 20 mg/kg, 21 mg/kg, 22 mg/kg, 23 mg/kg, 24 mg/kg, 25 mg/kg, 30 mg/kg, 35 mg/kg, 40 mg/kg, 45 mg/kg, 50 mg/kg, 55 mg/kg, 60 mg/kg, 65 mg/kg, 70 mg/kg, 75 mg/kg, 80 mg/kg, 85 mg/kg, 90 mg/kg, 95 mg/kg, 100 mg/kg, 105 mg/kg, 110 mg/kg, 115 mg/kg, 120 mg/kg, 125 mg/kg, 130 mg/kg, 135 mg/kg, 140 mg/kg, 145 mg/kg, 150 mg/kg, 155 mg/kg, 160 mg/kg, 165 mg/kg, 170 mg/kg, 175 mg/kg, 180 mg/kg, 185 mg/kg, 190 mg/kg, 195 mg/kg, 200 mg/kg, and any fraction or integer in between any two of the preceding amounts.

In some embodiments, a NOS inhibitor composition comprising L-NAME and/or a salt thereof, such as, for example, L-NAME hydrochloride, may be administered as a suitable dosage form, as described herein, to treat, ameliorate, and/or reduce the severity of S-SCC cancer or one or more symptoms thereof in an S-SCC cancer subject. In certain embodiments, an nNOS inhibitor composition comprising L-NAME and/or a salt thereof, such as, for example, L-NAME hydrochloride, may be administered to an S-SCC cancer subject to treat, ameliorate, and/or reduce the severity of S-SCC cancer or one or more symptoms thereof in the S-SCC cancer subject: (i) via a route selected from the group consisting of buccally, inhalation, intraarterially, intraarticularly, intracardially, intracerebroventricularly, intradermally, intramedullary, intramuscularly, intraperitoneally, intrathecally, intravenously, intraventricularly, nasally, ophthalmically, orally, rectally, sublabially, sublingually, subcutaneously, topically, and transdermally; and/or (ii) with a frequency selected from the group consisting of four times daily, three times daily, twice daily, once daily, once every two days, once every three days, once every four days, once every five days, once every six days, once a week, once every eight days, once every nine days, once every ten days, once every two weeks, once every three weeks, and once a month; and/or (iii) at a dosage (based on subject weight) selected from the group consisting of 0.1 μg/kg, 0.5 μg/kg, 1 μg/kg, 1.5 μg/kg, 2.0 μg/kg, 2.5 μg/kg, 3.0 μg/kg, 3.5 μg/kg, 4.0 μg/kg, 4.5 μg/kg, 5.0 μg/kg, 10 μg/kg, 15 μg/kg, 20 μg/kg, 25 μg/kg, 30 μg/kg, 35 μg/kg, 40 μg/kg, 45 μg/kg, 50 μg/kg, 55 μg/kg, 60 μg/kg, 65 μg/kg, 70 μg/kg, 75 μg/kg, 80 μg/kg, 85 μg/kg, 90 μg/kg, 95 μg/kg, 100 μg/kg, 150 μg/kg, 200 μg/kg, 250 μg/kg, 300 μg/kg, 350 μg/kg, 400 μg/kg, 450 μg/kg, 500 μg/kg, 550 μg/kg, 600 μg/kg, 650 μg/kg, 700 μg/kg, 750 μg/kg, 800 μg/kg, 850 μg/kg, 900 μg/kg, 1 mg/kg, 1.5 mg/kg, 2.0 mg/kg, 2.5 mg/kg, 3 mg/kg, 3.5 mg/kg, 4.0 mg/kg, 4.5 mg/kg, 5 mg/kg, 5.5 mg/kg, 6 mg/kg, 6.5 mg/kg, 7 mg/kg, 7.5 mg/kg, 8 mg/kg, 8.5 mg/kg, 9 mg/kg, 9.5 mg/kg, 10 mg/kg 10.5 mg/kg, 11 mg/kg, 11.5 mg/kg, 12 mg/kg, 12.5 mg/kg, 13 mg/kg, 13.5 mg/kg, 14 mg/kg, 14.5 mg/kg, 15 mg/kg, 16 mg/kg, 17 mg/kg, 18 mg/kg, 19 mg/kg, 20 mg/kg, 21 mg/kg, 22 mg/kg, 23 mg/kg, 24 mg/kg, 25 mg/kg, 30 mg/kg, 35 mg/kg, 40 mg/kg, 45 mg/kg, 50 mg/kg, 55 mg/kg, 60 mg/kg, 65 mg/kg, 70 mg/kg, 75 mg/kg, 80 mg/kg, 85 mg/kg, 90 mg/kg, 95 mg/kg, 100 mg/kg, 105 mg/kg, 110 mg/kg, 115 mg/kg, 120 mg/kg, 125 mg/kg, 130 mg/kg, 135 mg/kg, 140 mg/kg, 145 mg/kg, 150 mg/kg, 155 mg/kg, 160 mg/kg, 165 mg/kg, 170 mg/kg, 175 mg/kg, 180 mg/kg, 185 mg/kg, 190 mg/kg, 195 mg/kg, 200 mg/kg, and any fraction or integer in between any two of the preceding amounts.

In certain embodiments, a NOS inhibitor composition comprising L-NMMA and/or a salt thereof, such as, for example, L-NMMA acetate, may be administered as a suitable dosage form, as described herein, to treat, ameliorate, and/or reduce the severity of S-SCC cancer or one or more symptoms thereof in an S-SCC cancer subject. In some embodiments, an nNOS inhibitor composition comprising L-NMMA and/or a salt thereof, such as, for example, L-NMMA acetate, may be administered to an S-SCC cancer subject to treat, ameliorate, and/or reduce the severity of S-SCC cancer or one or more symptoms thereof in the S-SCC cancer subject: (i) via a route selected from the group consisting of buccally, inhalation, intraarterially, intraarticularly, intracardially, intracerebroventricularly, intradermally, intramedullary, intramuscularly, intraperitoneally, intrathecally, intravenously, intraventricularly, nasally, ophthalmically, orally, rectally, sublabially, sublingually, subcutaneously, topically, and transdermally; and/or (ii) with a frequency selected from the group consisting of four times daily, three times daily, twice daily, once daily, once every two days, once every three days, once every four days, once every five days, once every six days, once a week, once every eight days, once every nine days, once every ten days, once every two weeks, once every three weeks, and once a month; and/or (iii) at a dosage (based on subject weight) selected from the group consisting of 0.1 μg/kg, 0.5 μg/kg, 1 μg/kg, 1.5 μg/kg, 2.0 μg/kg, 2.5 μg/kg, 3.0 μg/kg, 3.5 μg/kg, 4.0 μg/kg, 4.5 μg/kg, 5.0 μg/kg, 10 μg/kg, 15 μg/kg, 20 μg/kg, 25 μg/kg, 30 μg/kg, 35 μg/kg, 40 μg/kg, 45 μg/kg, 50 μg/kg, 55 μg/kg, 60 μg/kg, 65 μg/kg, 70 μg/kg, 75 μg/kg, 80 μg/kg, 85 μg/kg, 90 μg/kg, 95 μg/kg, 100 μg/kg, 150 μg/kg, 200 μg/kg, 250 μg/kg, 300 μg/kg, 350 μg/kg, 400 μg/kg, 450 μg/kg, 500 μg/kg, 550 μg/kg, 600 μg/kg, 650 μg/kg, 700 μg/kg, 750 μg/kg, 800 μg/kg, 850 μg/kg, 900 μg/kg, 1 mg/kg, 1.5 mg/kg, 2.0 mg/kg, 2.5 mg/kg, 3 mg/kg, 3.5 mg/kg, 4.0 mg/kg, 4.5 mg/kg, 5 mg/kg, 5.5 mg/kg, 6 mg/kg, 6.5 mg/kg, 7 mg/kg, 7.5 mg/kg, 8 mg/kg, 8.5 mg/kg, 9 mg/kg, 9.5 mg/kg, 10 mg/kg 10.5 mg/kg, 11 mg/kg, 11.5 mg/kg, 12 mg/kg, 12.5 mg/kg, 13 mg/kg, 13.5 mg/kg, 14 mg/kg, 14.5 mg/kg, 15 mg/kg, 16 mg/kg, 17 mg/kg, 18 mg/kg, 19 mg/kg, 20 mg/kg, 21 mg/kg, 22 mg/kg, 23 mg/kg, 24 mg/kg, 25 mg/kg, 30 mg/kg, 35 mg/kg, 40 mg/kg, 45 mg/kg, 50 mg/kg, 55 mg/kg, 60 mg/kg, 65 mg/kg, 70 mg/kg, 75 mg/kg, 80 mg/kg, 85 mg/kg, 90 mg/kg, 95 mg/kg, 100 mg/kg, 105 mg/kg, 110 mg/kg, 115 mg/kg, 120 mg/kg, 125 mg/kg, 130 mg/kg, 135 mg/kg, 140 mg/kg, 145 mg/kg, 150 mg/kg, 155 mg/kg, 160 mg/kg, 165 mg/kg, 170 mg/kg, 175 mg/kg, 180 mg/kg, 185 mg/kg, 190 mg/kg, 195 mg/kg, 200 mg/kg, and any fraction or integer in between any two of the preceding amounts.

In some embodiments, a NOS inhibitor composition comprising GW-274150 and/or a salt thereof, such as, for example, GW-274150 phosphate, may be administered as a suitable dosage form, as described herein, to treat, ameliorate, and/or reduce the severity of S-SCC cancer or one or more symptoms thereof in an S-SCC cancer subject. In certain embodiments, an nNOS inhibitor composition comprising GW-274150 and/or a salt thereof, such as, for example, GW-274150 phosphate, may be administered to an S-SCC cancer subject to treat, ameliorate, and/or reduce the severity of S-SCC cancer or one or more symptoms thereof in the S-SCC cancer subject: (i) via a route selected from the group consisting of buccally, inhalation, intraarterially, intraarticularly, intracardially, intracerebroventricularly, intradermally, intramedullary, intramuscularly, intraperitoneally, intrathecally, intravenously, intraventricularly, nasally, ophthalmically, orally, rectally, sublabially, sublingually, subcutaneously, topically, and transdermally; and/or (ii) with a frequency selected from the group consisting of four times daily, three times daily, twice daily, once daily, once every two days, once every three days, once every four days, once every five days, once every six days, once a week, once every eight days, once every nine days, once every ten days, once every two weeks, once every three weeks, and once a month; and/or (iii) at a dosage (based on subject weight) selected from the group consisting of 0.1 μg/kg, 0.5 μg/kg, 1 μg/kg, 1.5 μg/kg, 2.0 μg/kg, 2.5 μg/kg, 3.0 μg/kg, 3.5 μg/kg, 4.0 μg/kg, 4.5 μg/kg, 5.0 μg/kg, 10 μg/kg, 15 μg/kg, 20 μg/kg, 25 μg/kg, 30 μg/kg, 35 μg/kg, 40 μg/kg, 45 μg/kg, 50 μg/kg, 55 μg/kg, 60 μg/kg, 65 μg/kg, 70 μg/kg, 75 μg/kg, 80 μg/kg, 85 μg/kg, 90 μg/kg, 95 μg/kg, 100 μg/kg, 150 μg/kg, 200 μg/kg, 250 μg/kg, 300 μg/kg, 350 μg/kg, 400 μg/kg, 450 μg/kg, 500 μg/kg, 550 μg/kg, 600 μg/kg, 650 μg/kg, 700 μg/kg, 750 μg/kg, 800 μg/kg, 850 μg/kg, 900 μg/kg, 1 mg/kg, 1.5 mg/kg, 2.0 mg/kg, 2.5 mg/kg, 3 mg/kg, 3.5 mg/kg, 4.0 mg/kg, 4.5 mg/kg, 5 mg/kg, 5.5 mg/kg, 6 mg/kg, 6.5 mg/kg, 7 mg/kg, 7.5 mg/kg, 8 mg/kg, 8.5 mg/kg, 9 mg/kg, 9.5 mg/kg, 10 mg/kg 10.5 mg/kg, 11 mg/kg, 11.5 mg/kg, 12 mg/kg, 12.5 mg/kg, 13 mg/kg, 13.5 mg/kg, 14 mg/kg, 14.5 mg/kg, 15 mg/kg, 16 mg/kg, 17 mg/kg, 18 mg/kg, 19 mg/kg, 20 mg/kg, 21 mg/kg, 22 mg/kg, 23 mg/kg, 24 mg/kg, 25 mg/kg, 30 mg/kg, 35 mg/kg, 40 mg/kg, 45 mg/kg, 50 mg/kg, 55 mg/kg, 60 mg/kg, 65 mg/kg, 70 mg/kg, 75 mg/kg, 80 mg/kg, 85 mg/kg, 90 mg/kg, 95 mg/kg, 100 mg/kg, 105 mg/kg, 110 mg/kg, 115 mg/kg, 120 mg/kg, 125 mg/kg, 130 mg/kg, 135 mg/kg, 140 mg/kg, 145 mg/kg, 150 mg/kg, 155 mg/kg, 160 mg/kg, 165 mg/kg, 170 mg/kg, 175 mg/kg, 180 mg/kg, 185 mg/kg, 190 mg/kg, 195 mg/kg, 200 mg/kg, and any fraction or integer in between any two of the preceding amounts.

In certain embodiments, a NOS inhibitor composition comprising L-NIL and/or a salt thereof, such as, for example, L-NIL hydrochloride, may be administered as a suitable dosage form, as described herein, to treat, ameliorate, and/or reduce the severity of S-SCC cancer or one or more symptoms thereof in an S-SCC cancer subject. In some embodiments, an nNOS inhibitor composition comprising L-NIL and/or a salt thereof, such as, for example, L-NIL hydrochloride, may be administered to an S-SCC cancer subject to treat, ameliorate, and/or reduce the severity of S-SCC cancer or one or more symptoms thereof in the S-SCC cancer subject: (i) via a route selected from the group consisting of buccally, inhalation, intraarterially, intraarticularly, intracardially, intracerebroventricularly, intradermally, intramedullary, intramuscularly, intraperitoneally, intrathecally, intravenously, intraventricularly, nasally, ophthalmically, orally, rectally, sublabially, sublingually, subcutaneously, topically, and transdermally; and/or (ii) with a frequency selected from the group consisting of four times daily, three times daily, twice daily, once daily, once every two days, once every three days, once every four days, once every five days, once every six days, once a week, once every eight days, once every nine days, once every ten days, once every two weeks, once every three weeks, and once a month; and/or (iii) at a dosage (based on subject weight) selected from the group consisting of 0.1 μg/kg, 0.5 μg/kg, 1 μg/kg, 1.5 μg/kg, 2.0 μg/kg, 2.5 μg/kg, 3.0 μg/kg, 3.5 μg/kg, 4.0 μg/kg, 4.5 μg/kg, 5.0 μg/kg, 10 μg/kg, 15 μg/kg, 20 μg/kg, 25 μg/kg, 30 μg/kg, 35 μg/kg, 40 μg/kg, 45 μg/kg, 50 μg/kg, 55 μg/kg, 60 μg/kg, 65 μg/kg, 70 μg/kg, 75 μg/kg, 80 μg/kg, 85 μg/kg, 90 μg/kg, 95 μg/kg, 100 μg/kg, 150 μg/kg, 200 μg/kg, 250 μg/kg, 300 μg/kg, 350 μg/kg, 400 μg/kg, 450 μg/kg, 500 μg/kg, 550 μg/kg, 600 μg/kg, 650 μg/kg, 700 μg/kg, 750 μg/kg, 800 μg/kg, 850 μg/kg, 900 μg/kg, 1 mg/kg, 1.5 mg/kg, 2.0 mg/kg, 2.5 mg/kg, 3 mg/kg, 3.5 mg/kg, 4.0 mg/kg, 4.5 mg/kg, 5 mg/kg, 5.5 mg/kg, 6 mg/kg, 6.5 mg/kg, 7 mg/kg, 7.5 mg/kg, 8 mg/kg, 8.5 mg/kg, 9 mg/kg, 9.5 mg/kg, 10 mg/kg 10.5 mg/kg, 11 mg/kg, 11.5 mg/kg, 12 mg/kg, 12.5 mg/kg, 13 mg/kg, 13.5 mg/kg, 14 mg/kg, 14.5 mg/kg, 15 mg/kg, 16 mg/kg, 17 mg/kg, 18 mg/kg, 19 mg/kg, 20 mg/kg, 21 mg/kg, 22 mg/kg, 23 mg/kg, 24 mg/kg, 25 mg/kg, 30 mg/kg, 35 mg/kg, 40 mg/kg, 45 mg/kg, 50 mg/kg, 55 mg/kg, 60 mg/kg, 65 mg/kg, 70 mg/kg, 75 mg/kg, 80 mg/kg, 85 mg/kg, 90 mg/kg, 95 mg/kg, 100 mg/kg, 105 mg/kg, 110 mg/kg, 115 mg/kg, 120 mg/kg, 125 mg/kg, 130 mg/kg, 135 mg/kg, 140 mg/kg, 145 mg/kg, 150 mg/kg, 155 mg/kg, 160 mg/kg, 165 mg/kg, 170 mg/kg, 175 mg/kg, 180 mg/kg, 185 mg/kg, 190 mg/kg, 195 mg/kg, 200 mg/kg, and any fraction or integer in between any two of the preceding amounts.

In some embodiments, a NOS inhibitor composition comprising 1400W and/or a salt thereof, such as, for example, 1400W dihydrochloride, may be administered as a suitable dosage form, as described herein, to treat, ameliorate, and/or reduce the severity of S-SCC cancer or one or more symptoms thereof in an S-SCC cancer subject. In certain embodiments, an nNOS inhibitor composition comprising 1400W and/or a salt thereof, such as, for example, 1400W dihydrochloride, may be administered to an S-SCC cancer subject to treat, ameliorate, and/or reduce the severity of S-SCC cancer or one or more symptoms thereof in the S-SCC cancer subject: (i) via a route selected from the group consisting of buccally, inhalation, intraarterially, intraarticularly, intracardially, intracerebroventricularly, intradermally, intramedullary, intramuscularly, intraperitoneally, intrathecally, intravenously, intraventricularly, nasally, ophthalmically, orally, rectally, sublabially, sublingually, subcutaneously, topically, and transdermally; and/or (ii) with a frequency selected from the group consisting of four times daily, three times daily, twice daily, once daily, once every two days, once every three days, once every four days, once every five days, once every six days, once a week, once every eight days, once every nine days, once every ten days, once every two weeks, once every three weeks, and once a month; and/or (iii) at a dosage (based on subject weight) selected from the group consisting of 0.1 μg/kg, 0.5 μg/kg, 1 μg/kg, 1.5 μg/kg, 2.0 μg/kg, 2.5 μg/kg, 3.0 μg/kg, 3.5 μg/kg, 4.0 μg/kg, 4.5 μg/kg, 5.0 μg/kg, 10 μg/kg, 15 μg/kg, 20 μg/kg, 25 μg/kg, 30 μg/kg, 35 μg/kg, 40 μg/kg, 45 μg/kg, 50 μg/kg, 55 μg/kg, 60 μg/kg, 65 μg/kg, 70 μg/kg, 75 μg/kg, 80 μg/kg, 85 μg/kg, 90 μg/kg, 95 μg/kg, 100 μg/kg, 150 μg/kg, 200 μg/kg, 250 μg/kg, 300 μg/kg, 350 μg/kg, 400 μg/kg, 450 μg/kg, 500 μg/kg, 550 μg/kg, 600 μg/kg, 650 μg/kg, 700 μg/kg, 750 μg/kg, 800 μg/kg, 850 μg/kg, 900 μg/kg, 1 mg/kg, 1.5 mg/kg, 2.0 mg/kg, 2.5 mg/kg, 3 mg/kg, 3.5 mg/kg, 4.0 mg/kg, 4.5 mg/kg, 5 mg/kg, 5.5 mg/kg, 6 mg/kg, 6.5 mg/kg, 7 mg/kg, 7.5 mg/kg, 8 mg/kg, 8.5 mg/kg, 9 mg/kg, 9.5 mg/kg, 10 mg/kg 10.5 mg/kg, 11 mg/kg, 11.5 mg/kg, 12 mg/kg, 12.5 mg/kg, 13 mg/kg, 13.5 mg/kg, 14 mg/kg, 14.5 mg/kg, 15 mg/kg, 16 mg/kg, 17 mg/kg, 18 mg/kg, 19 mg/kg, 20 mg/kg, 21 mg/kg, 22 mg/kg, 23 mg/kg, 24 mg/kg, 25 mg/kg, 30 mg/kg, 35 mg/kg, 40 mg/kg, 45 mg/kg, 50 mg/kg, 55 mg/kg, 60 mg/kg, 65 mg/kg, 70 mg/kg, 75 mg/kg, 80 mg/kg, 85 mg/kg, 90 mg/kg, 95 mg/kg, 100 mg/kg, 105 mg/kg, 110 mg/kg, 115 mg/kg, 120 mg/kg, 125 mg/kg, 130 mg/kg, 135 mg/kg, 140 mg/kg, 145 mg/kg, 150 mg/kg, 155 mg/kg, 160 mg/kg, 165 mg/kg, 170 mg/kg, 175 mg/kg, 180 mg/kg, 185 mg/kg, 190 mg/kg, 195 mg/kg, 200 mg/kg, and any fraction or integer in between any two of the preceding amounts.

In certain embodiments, a NOS inhibitor composition comprising OKN-007 and/or a salt thereof, such as, for example, OKN-007 sodium, may be administered as a suitable dosage form, as described herein, to treat, ameliorate, and/or reduce the severity of S-SCC cancer or one or more symptoms thereof in an S-SCC cancer subject. In some embodiments, an nNOS inhibitor composition comprising OKN-007 and/or a salt thereof, such as, for example, OKN-007 sodium, may be administered to an S-SCC cancer subject to treat, ameliorate, and/or reduce the severity of S-SCC cancer or one or more symptoms thereof in the S-SCC cancer subject: (i) via a route selected from the group consisting of buccally, inhalation, intraarterially, intraarticularly, intracardially, intracerebroventricularly, intradermally, intramedullary, intramuscularly, intraperitoneally, intrathecally, intravenously, intraventricularly, nasally, ophthalmically, orally, rectally, sublabially, sublingually, subcutaneously, topically, and transdermally; and/or (ii) with a frequency selected from the group consisting of four times daily, three times daily, twice daily, once daily, once every two days, once every three days, once every four days, once every five days, once every six days, once a week, once every eight days, once every nine days, once every ten days, once every two weeks, once every three weeks, and once a month; and/or (iii) at a dosage (based on subject weight) selected from the group consisting of 0.1 μg/kg, 0.5 μg/kg, 1 μg/kg, 1.5 μg/kg, 2.0 μg/kg, 2.5 μg/kg, 3.0 μg/kg, 3.5 μg/kg, 4.0 μg/kg, 4.5 μg/kg, 5.0 μg/kg, 10 μg/kg, 15 μg/kg, 20 μg/kg, 25 μg/kg, 30 μg/kg, 35 μg/kg, 40 μg/kg, 45 μg/kg, 50 μg/kg, 55 μg/kg, 60 μg/kg, 65 μg/kg, 70 μg/kg, 75 μg/kg, 80 μg/kg, 85 μg/kg, 90 μg/kg, 95 μg/kg, 100 μg/kg, 150 μg/kg, 200 μg/kg, 250 μg/kg, 300 μg/kg, 350 μg/kg, 400 μg/kg, 450 μg/kg, 500 μg/kg, 550 μg/kg, 600 μg/kg, 650 μg/kg, 700 μg/kg, 750 μg/kg, 800 μg/kg, 850 μg/kg, 900 μg/kg, 1 mg/kg, 1.5 mg/kg, 2.0 mg/kg, 2.5 mg/kg, 3 mg/kg, 3.5 mg/kg, 4.0 mg/kg, 4.5 mg/kg, 5 mg/kg, 5.5 mg/kg, 6 mg/kg, 6.5 mg/kg, 7 mg/kg, 7.5 mg/kg, 8 mg/kg, 8.5 mg/kg, 9 mg/kg, 9.5 mg/kg, 10 mg/kg 10.5 mg/kg, 11 mg/kg, 11.5 mg/kg, 12 mg/kg, 12.5 mg/kg, 13 mg/kg, 13.5 mg/kg, 14 mg/kg, 14.5 mg/kg, 15 mg/kg, 16 mg/kg, 17 mg/kg, 18 mg/kg, 19 mg/kg, 20 mg/kg, 21 mg/kg, 22 mg/kg, 23 mg/kg, 24 mg/kg, 25 mg/kg, 30 mg/kg, 35 mg/kg, 40 mg/kg, 45 mg/kg, 50 mg/kg, 55 mg/kg, 60 mg/kg, 65 mg/kg, 70 mg/kg, 75 mg/kg, 80 mg/kg, 85 mg/kg, 90 mg/kg, 95 mg/kg, 100 mg/kg, 105 mg/kg, 110 mg/kg, 115 mg/kg, 120 mg/kg, 125 mg/kg, 130 mg/kg, 135 mg/kg, 140 mg/kg, 145 mg/kg, 150 mg/kg, 155 mg/kg, 160 mg/kg, 165 mg/kg, 170 mg/kg, 175 mg/kg, 180 mg/kg, 185 mg/kg, 190 mg/kg, 195 mg/kg, 200 mg/kg, and any fraction or integer in between any two of the preceding amounts.

In some embodiments, a NOS inhibitor composition comprising BYK-191023 and/or a salt thereof, such as, for example, BYK-191023 dihydrochloride, may be administered as a suitable dosage form, as described herein, to treat, ameliorate, and/or reduce the severity of S-SCC cancer or one or more symptoms thereof in an S-SCC cancer subject. In certain embodiments, an nNOS inhibitor composition comprising BYK-191023 and/or a salt thereof, such as, for example, BYK-191023 dihydrochloride, may be administered to an S-SCC cancer subject to treat, ameliorate, and/or reduce the severity of S-SCC cancer or one or more symptoms thereof in the S-SCC cancer subject: (i) via a route selected from the group consisting of buccally, inhalation, intraarterially, intraarticularly, intracardially, intracerebroventricularly, intradermally, intramedullary, intramuscularly, intraperitoneally, intrathecally, intravenously, intraventricularly, nasally, ophthalmically, orally, rectally, sublabially, sublingually, subcutaneously, topically, and transdermally; and/or (ii) with a frequency selected from the group consisting of four times daily, three times daily, twice daily, once daily, once every two days, once every three days, once every four days, once every five days, once every six days, once a week, once every eight days, once every nine days, once every ten days, once every two weeks, once every three weeks, and once a month; and/or (iii) at a dosage (based on subject weight) selected from the group consisting of 0.1 μg/kg, 0.5 μg/kg, 1 μg/kg, 1.5 μg/kg, 2.0 μg/kg, 2.5 μg/kg, 3.0 μg/kg, 3.5 μg/kg, 4.0 μg/kg, 4.5 μg/kg, 5.0 μg/kg, 10 μg/kg, 15 μg/kg, 20 μg/kg, 25 μg/kg, 30 μg/kg, 35 μg/kg, 40 μg/kg, 45 μg/kg, 50 μg/kg, 55 μg/kg, 60 μg/kg, 65 μg/kg, 70 μg/kg, 75 μg/kg, 80 μg/kg, 85 μg/kg, 90 μg/kg, 95 μg/kg, 100 μg/kg, 150 μg/kg, 200 μg/kg, 250 μg/kg, 300 μg/kg, 350 μg/kg, 400 μg/kg, 450 μg/kg, 500 μg/kg, 550 μg/kg, 600 μg/kg, 650 μg/kg, 700 μg/kg, 750 μg/kg, 800 μg/kg, 850 μg/kg, 900 μg/kg, 1 mg/kg, 1.5 mg/kg, 2.0 mg/kg, 2.5 mg/kg, 3 mg/kg, 3.5 mg/kg, 4.0 mg/kg, 4.5 mg/kg, 5 mg/kg, 5.5 mg/kg, 6 mg/kg, 6.5 mg/kg, 7 mg/kg, 7.5 mg/kg, 8 mg/kg, 8.5 mg/kg, 9 mg/kg, 9.5 mg/kg, 10 mg/kg 10.5 mg/kg, 11 mg/kg, 11.5 mg/kg, 12 mg/kg, 12.5 mg/kg, 13 mg/kg, 13.5 mg/kg, 14 mg/kg, 14.5 mg/kg, 15 mg/kg, 16 mg/kg, 17 mg/kg, 18 mg/kg, 19 mg/kg, 20 mg/kg, 21 mg/kg, 22 mg/kg, 23 mg/kg, 24 mg/kg, 25 mg/kg, 30 mg/kg, 35 mg/kg, 40 mg/kg, 45 mg/kg, 50 mg/kg, 55 mg/kg, 60 mg/kg, 65 mg/kg, 70 mg/kg, 75 mg/kg, 80 mg/kg, 85 mg/kg, 90 mg/kg, 95 mg/kg, 100 mg/kg, 105 mg/kg, 110 mg/kg, 115 mg/kg, 120 mg/kg, 125 mg/kg, 130 mg/kg, 135 mg/kg, 140 mg/kg, 145 mg/kg, 150 mg/kg, 155 mg/kg, 160 mg/kg, 165 mg/kg, 170 mg/kg, 175 mg/kg, 180 mg/kg, 185 mg/kg, 190 mg/kg, 195 mg/kg, 200 mg/kg, and any fraction or integer in between any two of the preceding amounts.

In certain embodiments, a NOS inhibitor composition comprising CM544 and/or a salt thereof, such as, for example, CM544 dihydrochloride, may be administered as a suitable dosage form, as described herein, to treat, ameliorate, and/or reduce the severity of S-SCC cancer or one or more symptoms thereof in an S-SCC cancer subject. In some embodiments, an nNOS inhibitor composition comprising CM544 and/or a salt thereof, such as, for example, CM544 dihydrochloride, may be administered to an S-SCC cancer subject to treat, ameliorate, and/or reduce the severity of S-SCC cancer or one or more symptoms thereof in the S-SCC cancer subject: (i) via a route selected from the group consisting of buccally, inhalation, intraarterially, intraarticularly, intracardially, intracerebroventricularly, intradermally, intramedullary, intramuscularly, intraperitoneally, intrathecally, intravenously, intraventricularly, nasally, ophthalmically, orally, rectally, sublabially, sublingually, subcutaneously, topically, and transdermally; and/or (ii) with a frequency selected from the group consisting of four times daily, three times daily, twice daily, once daily, once every two days, once every three days, once every four days, once every five days, once every six days, once a week, once every eight days, once every nine days, once every ten days, once every two weeks, once every three weeks, and once a month; and/or (iii) at a dosage (based on subject weight) selected from the group consisting of 0.1 μg/kg, 0.5 μg/kg, 1 μg/kg, 1.5 μg/kg, 2.0 μg/kg, 2.5 μg/kg, 3.0 μg/kg, 3.5 μg/kg, 4.0 μg/kg, 4.5 μg/kg, 5.0 μg/kg, 10 μg/kg, 15 μg/kg, 20 μg/kg, 25 μg/kg, 30 μg/kg, 35 μg/kg, 40 μg/kg, 45 μg/kg, 50 μg/kg, 55 μg/kg, 60 μg/kg, 65 μg/kg, 70 μg/kg, 75 μg/kg, 80 μg/kg, 85 μg/kg, 90 μg/kg, 95 μg/kg, 100 μg/kg, 150 μg/kg, 200 μg/kg, 250 μg/kg, 300 μg/kg, 350 μg/kg, 400 μg/kg, 450 μg/kg, 500 μg/kg, 550 μg/kg, 600 μg/kg, 650 μg/kg, 700 μg/kg, 750 μg/kg, 800 μg/kg, 850 μg/kg, 900 μg/kg, 1 mg/kg, 1.5 mg/kg, 2.0 mg/kg, 2.5 mg/kg, 3 mg/kg, 3.5 mg/kg, 4.0 mg/kg, 4.5 mg/kg, 5 mg/kg, 5.5 mg/kg, 6 mg/kg, 6.5 mg/kg, 7 mg/kg, 7.5 mg/kg, 8 mg/kg, 8.5 mg/kg, 9 mg/kg, 9.5 mg/kg, 10 mg/kg 10.5 mg/kg, 11 mg/kg, 11.5 mg/kg, 12 mg/kg, 12.5 mg/kg, 13 mg/kg, 13.5 mg/kg, 14 mg/kg, 14.5 mg/kg, 15 mg/kg, 16 mg/kg, 17 mg/kg, 18 mg/kg, 19 mg/kg, 20 mg/kg, 21 mg/kg, 22 mg/kg, 23 mg/kg, 24 mg/kg, 25 mg/kg, 30 mg/kg, 35 mg/kg, 40 mg/kg, 45 mg/kg, 50 mg/kg, 55 mg/kg, 60 mg/kg, 65 mg/kg, 70 mg/kg, 75 mg/kg, 80 mg/kg, 85 mg/kg, 90 mg/kg, 95 mg/kg, 100 mg/kg, 105 mg/kg, 110 mg/kg, 115 mg/kg, 120 mg/kg, 125 mg/kg, 130 mg/kg, 135 mg/kg, 140 mg/kg, 145 mg/kg, 150 mg/kg, 155 mg/kg, 160 mg/kg, 165 mg/kg, 170 mg/kg, 175 mg/kg, 180 mg/kg, 185 mg/kg, 190 mg/kg, 195 mg/kg, 200 mg/kg, and any fraction or integer in between any two of the preceding amounts.

In certain embodiments, an nNOS inhibitor composition comprising one or more dsRNAs each comprising at least 30 base pairs, may be administered as a suitable dosage form, as described herein, to treat, ameliorate, and/or reduce the severity of S-SCC cancer or one or more symptoms thereof in an S-SCC cancer subject. In some embodiments, an nNOS inhibitor composition comprising one or more dsRNAs, each comprising at least 30 base pairs, may be administered to an S-SCC cancer subject to treat, ameliorate, and/or reduce the severity of S-SCC cancer or one or more symptoms thereof in the S-SCC cancer subject: (i) via a route selected from the group consisting of buccally, inhalation, intraarterially, intraarticularly, intracardially, intracerebroventricularly, intradermally, intramedullary, intramuscularly, intraperitoneally, intrathecally, intravenously, intraventricularly, nasally, ophthalmically, orally, rectally, sublabially, sublingually, subcutaneously, topically, and transdermally; and/or (ii) with a frequency selected from the group consisting of four times daily, three times daily, twice daily, once daily, once every two days, once every three days, once every four days, once every five days, once every six days, once a week, once every eight days, once every nine days, once every ten days, once every two weeks, once every three weeks, and once a month; and/or (iii) at a dosage (based on subject weight) selected from the group consisting of 0.1 μg/kg, 0.5 μg/kg, 1 μg/kg, 1.5 μg/kg, 2.0 μg/kg, 2.5 μg/kg, 3.0 μg/kg, 3.5 μg/kg, 4.0 μg/kg, 4.5 μg/kg, 5.0 μg/kg, 10 μg/kg, 15 μg/kg, 20 μg/kg, 25 μg/kg, 30 μg/kg, 35 μg/kg, 40 μg/kg, 45 μg/kg, 50 μg/kg, 55 μg/kg, 60 μg/kg, 65 μg/kg, 70 μg/kg, 75 μg/kg, 80 μg/kg, 85 μg/kg, 90 μg/kg, 95 μg/kg, 100 μg/kg, 150 μg/kg, 200 μg/kg, 250 μg/kg, 300 μg/kg, 350 μg/kg, 400 μg/kg, 450 μg/kg, 500 μg/kg, 550 μg/kg, 600 μg/kg, 650 μg/kg, 700 μg/kg, 750 μg/kg, 800 μg/kg, 850 μg/kg, 900 μg/kg, 1 mg/kg, 1.5 mg/kg, 2.0 mg/kg, 2.5 mg/kg, 3 mg/kg, 3.5 mg/kg, 4.0 mg/kg, 4.5 mg/kg, 5 mg/kg, 5.5 mg/kg, 6 mg/kg, 6.5 mg/kg, 7 mg/kg, 7.5 mg/kg, 8 mg/kg, 8.5 mg/kg, 9 mg/kg, 9.5 mg/kg, 10 mg/kg 10.5 mg/kg, 11 mg/kg, 11.5 mg/kg, 12 mg/kg, 12.5 mg/kg, 13 mg/kg, 13.5 mg/kg, 14 mg/kg, 14.5 mg/kg, 15 mg/kg, 16 mg/kg, 17 mg/kg, 18 mg/kg, 19 mg/kg, 20 mg/kg, 21 mg/kg, 22 mg/kg, 23 mg/kg, 24 mg/kg, 25 mg/kg, 30 mg/kg, 35 mg/kg, 40 mg/kg, 45 mg/kg, 50 mg/kg, 55 mg/kg, 60 mg/kg, 65 mg/kg, 70 mg/kg, 75 mg/kg, 80 mg/kg, 85 mg/kg, 90 mg/kg, 95 mg/kg, 100 mg/kg, 105 mg/kg, 110 mg/kg, 115 mg/kg, 120 mg/kg, 125 mg/kg, 130 mg/kg, 135 mg/kg, 140 mg/kg, 145 mg/kg, 150 mg/kg, 155 mg/kg, 160 mg/kg, 165 mg/kg, 170 mg/kg, 175 mg/kg, 180 mg/kg, 185 mg/kg, 190 mg/kg, 195 mg/kg, 200 mg/kg, and any fraction or integer in between any two of the preceding amounts.

In some embodiments, an nNOS inhibitor composition comprising one or more siRNAs, may be administered as a suitable dosage form, as described herein, to treat, ameliorate, and/or reduce the severity of S-SCC cancer or one or more symptoms thereof in an S-SCC cancer subject. In certain embodiments, an nNOS inhibitor composition comprising one or more siRNAs selected from the group consisting of SEQ ID NO. 4, SEQ ID NO. 5, SEQ ID NO. 6, SEQ ID NO. 7, SEQ ID NO. 8, and any combination thereof, may be administered as a suitable dosage form, as described herein, to treat, ameliorate, and/or reduce the severity of S-SCC cancer or one or more symptoms thereof in an S-SCC cancer subject. In some embodiments, an nNOS inhibitor composition comprising one or more siRNAs selected from the group consisting of SEQ ID NO. 4, SEQ ID NO. 5, SEQ ID NO. 6, SEQ ID NO. 7, SEQ ID NO. 8, and any combination thereof, may be administered to an S-SCC cancer subject to treat, ameliorate, and/or reduce the severity of S-SCC cancer or one or more symptoms thereof in the S-SCC cancer subject: (i) via a route selected from the group consisting of buccally, inhalation, intraarterially, intraarticularly, intracardially, intracerebroventricularly, intradermally, intramedullary, intramuscularly, intraperitoneally, intrathecally, intravenously, intraventricularly, nasally, ophthalmically, orally, rectally, sublabially, sublingually, subcutaneously, topically, and transdermally; and/or (ii) with a frequency selected from the group consisting of four times daily, three times daily, twice daily, once daily, once every two days, once every three days, once every four days, once every five days, once every six days, once a week, once every eight days, once every nine days, once every ten days, once every two weeks, once every three weeks, and once a month; and/or (iii) at a dosage (based on subject weight) selected from the group consisting of 0.1 μg/kg, 0.5 μg/kg, 1 μg/kg, 1.5 μg/kg, 2.0 μg/kg, 2.5 μg/kg, 3.0 μg/kg, 3.5 μg/kg, 4.0 μg/kg, 4.5 μg/kg, 5.0 μg/kg, 10 μg/kg, 15 μg/kg, 20 μg/kg, 25 μg/kg, 30 μg/kg, 35 μg/kg, 40 μg/kg, 45 μg/kg, 50 μg/kg, 55 μg/kg, 60 μg/kg, 65 μg/kg, 70 μg/kg, 75 μg/kg, 80 μg/kg, 85 μg/kg, 90 μg/kg, 95 μg/kg, 100 μg/kg, 150 μg/kg, 200 μg/kg, 250 μg/kg, 300 μg/kg, 350 μg/kg, 400 μg/kg, 450 μg/kg, 500 μg/kg, 550 μg/kg, 600 μg/kg, 650 μg/kg, 700 μg/kg, 750 μg/kg, 800 μg/kg, 850 μg/kg, 900 μg/kg, 1 mg/kg, 1.5 mg/kg, 2.0 mg/kg, 2.5 mg/kg, 3 mg/kg, 3.5 mg/kg, 4.0 mg/kg, 4.5 mg/kg, 5 mg/kg, 5.5 mg/kg, 6 mg/kg, 6.5 mg/kg, 7 mg/kg, 7.5 mg/kg, 8 mg/kg, 8.5 mg/kg, 9 mg/kg, 9.5 mg/kg, 10 mg/kg 10.5 mg/kg, 11 mg/kg, 11.5 mg/kg, 12 mg/kg, 12.5 mg/kg, 13 mg/kg, 13.5 mg/kg, 14 mg/kg, 14.5 mg/kg, 15 mg/kg, 16 mg/kg, 17 mg/kg, 18 mg/kg, 19 mg/kg, 20 mg/kg, 21 mg/kg, 22 mg/kg, 23 mg/kg, 24 mg/kg, 25 mg/kg, 30 mg/kg, 35 mg/kg, 40 mg/kg, 45 mg/kg, 50 mg/kg, 55 mg/kg, 60 mg/kg, 65 mg/kg, 70 mg/kg, 75 mg/kg, 80 mg/kg, 85 mg/kg, 90 mg/kg, 95 mg/kg, 100 mg/kg, 105 mg/kg, 110 mg/kg, 115 mg/kg, 120 mg/kg, 125 mg/kg, 130 mg/kg, 135 mg/kg, 140 mg/kg, 145 mg/kg, 150 mg/kg, 155 mg/kg, 160 mg/kg, 165 mg/kg, 170 mg/kg, 175 mg/kg, 180 mg/kg, 185 mg/kg, 190 mg/kg, 195 mg/kg, 200 mg/kg, and any fraction or integer in between any two of the preceding amounts.

In some embodiments, an nNOS inhibitor composition comprising one or more shRNAs, may be administered as a suitable dosage form, as described herein, to treat, ameliorate, and/or reduce the severity of S-SCC cancer or one or more symptoms thereof in an S-SCC cancer subject. In certain embodiments, an nNOS inhibitor composition comprising one or more shRNAs selected from the group consisting of SEQ ID NO. 9, SEQ ID NO. 10, SEQ ID NO. 11, SEQ ID NO. 12, SEQ ID NO. 13, SEQ ID NO. 14, and any combination thereof, may be administered as a suitable dosage form, as described herein, to treat, ameliorate, and/or reduce the severity of S-SCC cancer or one or more symptoms thereof in an S-SCC cancer subject. In some embodiments, an nNOS inhibitor composition comprising one or more shRNAs selected from the group consisting of SEQ ID NO. 9, SEQ ID NO. 10, SEQ ID NO. 11, SEQ ID NO. 12, SEQ ID NO. 13, SEQ ID NO. 14, and any combination thereof, may be administered to an S-SCC cancer subject to treat, ameliorate, and/or reduce the severity of S-SCC cancer or one or more symptoms thereof in the S-SCC cancer subject: (i) via a route selected from the group consisting of buccally, inhalation, intraarterially, intraarticularly, intracardially, intracerebroventricularly, intradermally, intramedullary, intramuscularly, intraperitoneally, intrathecally, intravenously, intraventricularly, nasally, ophthalmically, orally, rectally, sublabially, sublingually, subcutaneously, topically, and transdermally; and/or (ii) with a frequency selected from the group consisting of four times daily, three times daily, twice daily, once daily, once every two days, once every three days, once every four days, once every five days, once every six days, once a week, once every eight days, once every nine days, once every ten days, once every two weeks, once every three weeks, and once a month; and/or (iii) at a dosage (based on subject weight) selected from the group consisting of 0.1 μg/kg, 0.5 μg/kg, 1 μg/kg, 1.5 μg/kg, 2.0 μg/kg, 2.5 μg/kg, 3.0 μg/kg, 3.5 μg/kg, 4.0 μg/kg, 4.5 μg/kg, 5.0 μg/kg, 10 μg/kg, 15 μg/kg, 20 μg/kg, 25 μg/kg, 30 μg/kg, 35 μg/kg, 40 μg/kg, 45 μg/kg, 50 μg/kg, 55 μg/kg, 60 μg/kg, 65 μg/kg, 70 μg/kg, 75 μg/kg, 80 μg/kg, 85 μg/kg, 90 μg/kg, 95 μg/kg, 100 μg/kg, 150 μg/kg, 200 μg/kg, 250 μg/kg, 300 μg/kg, 350 μg/kg, 400 μg/kg, 450 μg/kg, 500 μg/kg, 550 μg/kg, 600 μg/kg, 650 μg/kg, 700 μg/kg, 750 μg/kg, 800 μg/kg, 850 μg/kg, 900 μg/kg, 1 mg/kg, 1.5 mg/kg, 2.0 mg/kg, 2.5 mg/kg, 3 mg/kg, 3.5 mg/kg, 4.0 mg/kg, 4.5 mg/kg, 5 mg/kg, 5.5 mg/kg, 6 mg/kg, 6.5 mg/kg, 7 mg/kg, 7.5 mg/kg, 8 mg/kg, 8.5 mg/kg, 9 mg/kg, 9.5 mg/kg, 10 mg/kg 10.5 mg/kg, 11 mg/kg, 11.5 mg/kg, 12 mg/kg, 12.5 mg/kg, 13 mg/kg, 13.5 mg/kg, 14 mg/kg, 14.5 mg/kg, 15 mg/kg, 16 mg/kg, 17 mg/kg, 18 mg/kg, 19 mg/kg, 20 mg/kg, 21 mg/kg, 22 mg/kg, 23 mg/kg, 24 mg/kg, 25 mg/kg, 30 mg/kg, 35 mg/kg, 40 mg/kg, 45 mg/kg, 50 mg/kg, 55 mg/kg, 60 mg/kg, 65 mg/kg, 70 mg/kg, 75 mg/kg, 80 mg/kg, 85 mg/kg, 90 mg/kg, 95 mg/kg, 100 mg/kg, 105 mg/kg, 110 mg/kg, 115 mg/kg, 120 mg/kg, 125 mg/kg, 130 mg/kg, 135 mg/kg, 140 mg/kg, 145 mg/kg, 150 mg/kg, 155 mg/kg, 160 mg/kg, 165 mg/kg, 170 mg/kg, 175 mg/kg, 180 mg/kg, 185 mg/kg, 190 mg/kg, 195 mg/kg, 200 mg/kg, and any fraction or integer in between any two of the preceding amounts.

In some embodiments, an nNOS inhibitor composition comprising one or more miRNAs, may be administered as a suitable dosage form, as described herein, to treat, ameliorate, and/or reduce the severity of S-SCC cancer or one or more symptoms thereof in an S-SCC cancer subject. In certain embodiments, an nNOS inhibitor composition comprising one or more miRNAs selected from the group consisting of miR-146a, hsa-miR-146a-5p, hsa-miR-1273f, hsa-miR-143-3p, hsa-miR-147a, hsa-miR-3622a-5p, hsa-miR-3911, hsa-miR-4269, hsa-miR-4708-5p, hsa-miR-4710, hsa-miR-4770, hsa-miR-4792, hsa-miR-574-5p, hsa-miR-6088, hsa-miR-644a, hsa-miR-6715b-5p, hsa-miR-6768-5p, hsa-miR-6867-5p, hsa-miR-7855-5p, hsa-miR-8485, and any combination thereof, may be administered as a suitable dosage form, as described herein, to treat, ameliorate, and/or reduce the severity of S-SCC cancer or one or more symptoms thereof in an S-SCC cancer subject. In some embodiments, an nNOS inhibitor composition comprising one or more miRNAs selected from the group consisting of miR-146a, hsa-miR-146a-5p, hsa-miR-1273f, hsa-miR-143-3p, hsa-miR-147a, hsa-miR-3622a-5p, hsa-miR-3911, hsa-miR-4269, hsa-miR-4708-5p, hsa-miR-4710, hsa-miR-4770, hsa-miR-4792, hsa-miR-574-5p, hsa-miR-6088, hsa-miR-644a, hsa-miR-6715b-5p, hsa-miR-6768-5p, hsa-miR-6867-5p, hsa-miR-7855-5p, hsa-miR-8485, and any combination thereof, may be administered to an S-SCC cancer subject to treat, ameliorate, and/or reduce the severity of S-SCC cancer or one or more symptoms thereof in the S-SCC cancer subject: (i) via a route selected from the group consisting of buccally, inhalation, intraarterially, intraarticularly, intracardially, intracerebroventricularly, intradermally, intramedullary, intramuscularly, intraperitoneally, intrathecally, intravenously, intraventricularly, nasally, ophthalmically, orally, rectally, sublabially, sublingually, subcutaneously, topically, and transdermally; and/or (ii) with a frequency selected from the group consisting of four times daily, three times daily, twice daily, once daily, once every two days, once every three days, once every four days, once every five days, once every six days, once a week, once every eight days, once every nine days, once every ten days, once every two weeks, once every three weeks, and once a month; and/or (iii) at a dosage (based on subject weight) selected from the group consisting of 0.1 μg/kg, 0.5 μg/kg, 1 μg/kg, 1.5 μg/kg, 2.0 μg/kg, 2.5 μg/kg, 3.0 μg/kg, 3.5 μg/kg, 4.0 μg/kg, 4.5 μg/kg, 5.0 μg/kg, 10 μg/kg, 15 μg/kg, 20 μg/kg, 25 μg/kg, 30 μg/kg, 35 μg/kg, 40 μg/kg, 45 μg/kg, 50 μg/kg, 55 μg/kg, 60 μg/kg, 65 μg/kg, 70 μg/kg, 75 μg/kg, 80 μg/kg, 85 μg/kg, 90 μg/kg, 95 μg/kg, 100 μg/kg, 150 μg/kg, 200 μg/kg, 250 μg/kg, 300 μg/kg, 350 μg/kg, 400 μg/kg, 450 μg/kg, 500 μg/kg, 550 μg/kg, 600 μg/kg, 650 μg/kg, 700 μg/kg, 750 μg/kg, 800 μg/kg, 850 μg/kg, 900 μg/kg, 1 mg/kg, 1.5 mg/kg, 2.0 mg/kg, 2.5 mg/kg, 3 mg/kg, 3.5 mg/kg, 4.0 mg/kg, 4.5 mg/kg, 5 mg/kg, 5.5 mg/kg, 6 mg/kg, 6.5 mg/kg, 7 mg/kg, 7.5 mg/kg, 8 mg/kg, 8.5 mg/kg, 9 mg/kg, 9.5 mg/kg, 10 mg/kg 10.5 mg/kg, 11 mg/kg, 11.5 mg/kg, 12 mg/kg, 12.5 mg/kg, 13 mg/kg, 13.5 mg/kg, 14 mg/kg, 14.5 mg/kg, 15 mg/kg, 16 mg/kg, 17 mg/kg, 18 mg/kg, 19 mg/kg, 20 mg/kg, 21 mg/kg, 22 mg/kg, 23 mg/kg, 24 mg/kg, 25 mg/kg, 30 mg/kg, 35 mg/kg, 40 mg/kg, 45 mg/kg, 50 mg/kg, 55 mg/kg, 60 mg/kg, 65 mg/kg, 70 mg/kg, 75 mg/kg, 80 mg/kg, 85 mg/kg, 90 mg/kg, 95 mg/kg, 100 mg/kg, 105 mg/kg, 110 mg/kg, 115 mg/kg, 120 mg/kg, 125 mg/kg, 130 mg/kg, 135 mg/kg, 140 mg/kg, 145 mg/kg, 150 mg/kg, 155 mg/kg, 160 mg/kg, 165 mg/kg, 170 mg/kg, 175 mg/kg, 180 mg/kg, 185 mg/kg, 190 mg/kg, 195 mg/kg, 200 mg/kg, and any fraction or integer in between any two of the preceding amounts.

In some embodiments, an nNOS inhibitor composition comprising one or more CRISPR/Cas systems, may be administered as a suitable dosage form, as described herein, to treat, ameliorate, and/or reduce the severity of S-SCC cancer or one or more symptoms thereof in an S-SCC cancer subject. In certain embodiments, an nNOS inhibitor composition comprising one or more CRISPR/Cas systems comprising an endonuclease selected from the group consisting of Cas1, Cas2, Cas3, Cas4, Cas5, Cas6, Cas7, Cas8a, Cas8b, Cas8c, Cas9, Cas10, Cas10d, Cas12, Cas12a, Cas12b, Cas12c, Cas12d, Cas12e, Cas12f, Cas12g, Cas12h, Cas12i, Cas12k, C2c4, C2c8, C2c9, Cas13, Cas13a, Cas13b, Cas13c, Cas13d, Cas13x.1, Cse1, Cse2, Csy1, Csy2, Csy3, GSU0054, Csm2, Cmr5, Csx10, Csx11, Csf1, and Csn2, may be administered as a suitable dosage form, as described herein, to treat, ameliorate, and/or reduce the severity of S-SCC cancer or one or more symptoms thereof in an S-SCC cancer subject. In some embodiments, an nNOS inhibitor composition comprising one or more CRISPR/Cas systems comprising an endonuclease selected from the group consisting of Cas1, Cas2, Cas3, Cas4, Cas5, Cas6, Cas7, Cas8a, Cas8b, Cas8c, Cas9, Cas10, Cas10d, Cas12, Cas12a, Cas12b, Cas12c, Cas12d, Cas12e, Cas12f, Cas12g, Cas12h, Cas12i, Cas12k, C2c4, C2c8, C2c9, Cas13, Cas13a, Cas13b, Cas13c, Cas13d, Cas13x.1, Cse1, Cse2, Csy1, Csy2, Csy3, GSU0054, Csm2, Cmr5, Csx10, Csx11, Csf1, and Csn2, may be administered to an S-SCC cancer subject to treat, ameliorate, and/or reduce the severity of S-SCC cancer or one or more symptoms thereof in the S-SCC cancer subject: (i) via a route selected from the group consisting of buccally, inhalation, intraarterially, intraarticularly, intracardially, intracerebroventricularly, intradermally, intramedullary, intramuscularly, intraperitoneally, intrathecally, intravenously, intraventricularly, nasally, ophthalmically, orally, rectally, sublabially, sublingually, subcutaneously, topically, and transdermally; and/or (ii) with a frequency selected from the group consisting of four times daily, three times daily, twice daily, once daily, once every two days, once every three days, once every four days, once every five days, once every six days, once a week, once every eight days, once every nine days, once every ten days, once every two weeks, once every three weeks, and once a month; and/or (iii) at a dosage (based on subject weight) selected from the group consisting of 0.1 μg/kg, 0.5 μg/kg, 1 μg/kg, 1.5 μg/kg, 2.0 μg/kg, 2.5 μg/kg, 3.0 μg/kg, 3.5 μg/kg, 4.0 μg/kg, 4.5 μg/kg, 5.0 μg/kg, 10 μg/kg, 15 μg/kg, 20 μg/kg, 25 μg/kg, 30 μg/kg, 35 μg/kg, 40 μg/kg, 45 μg/kg, 50 μg/kg, 55 μg/kg, 60 μg/kg, 65 μg/kg, 70 μg/kg, 75 μg/kg, 80 μg/kg, 85 μg/kg, 90 μg/kg, 95 μg/kg, 100 μg/kg, 150 μg/kg, 200 μg/kg, 250 μg/kg, 300 μg/kg, 350 μg/kg, 400 μg/kg, 450 μg/kg, 500 μg/kg, 550 μg/kg, 600 μg/kg, 650 μg/kg, 700 μg/kg, 750 μg/kg, 800 μg/kg, 850 μg/kg, 900 μg/kg, 1 mg/kg, 1.5 mg/kg, 2.0 mg/kg, 2.5 mg/kg, 3 mg/kg, 3.5 mg/kg, 4.0 mg/kg, 4.5 mg/kg, 5 mg/kg, 5.5 mg/kg, 6 mg/kg, 6.5 mg/kg, 7 mg/kg, 7.5 mg/kg, 8 mg/kg, 8.5 mg/kg, 9 mg/kg, 9.5 mg/kg, 10 mg/kg 10.5 mg/kg, 11 mg/kg, 11.5 mg/kg, 12 mg/kg, 12.5 mg/kg, 13 mg/kg, 13.5 mg/kg, 14 mg/kg, 14.5 mg/kg, 15 mg/kg, 16 mg/kg, 17 mg/kg, 18 mg/kg, 19 mg/kg, 20 mg/kg, 21 mg/kg, 22 mg/kg, 23 mg/kg, 24 mg/kg, 25 mg/kg, 30 mg/kg, 35 mg/kg, 40 mg/kg, 45 mg/kg, 50 mg/kg, 55 mg/kg, 60 mg/kg, 65 mg/kg, 70 mg/kg, 75 mg/kg, 80 mg/kg, 85 mg/kg, 90 mg/kg, 95 mg/kg, 100 mg/kg, 105 mg/kg, 110 mg/kg, 115 mg/kg, 120 mg/kg, 125 mg/kg, 130 mg/kg, 135 mg/kg, 140 mg/kg, 145 mg/kg, 150 mg/kg, 155 mg/kg, 160 mg/kg, 165 mg/kg, 170 mg/kg, 175 mg/kg, 180 mg/kg, 185 mg/kg, 190 mg/kg, 195 mg/kg, 200 mg/kg, and any fraction or integer in between any two of the preceding amounts.

In some embodiments, an nNOS inhibitor composition comprising one or more antibodies, may be administered as a suitable dosage form, as described herein, to treat, ameliorate, and/or reduce the severity of S-SCC cancer or one or more symptoms thereof in an S-SCC cancer subject. In certain embodiments, an nNOS inhibitor composition comprising one or more antibodies selected from the group consisting of 37-2800 (from ThermoFisher), EP1855Y (from abeam), EPR 24351-6 (from abeam), and any combination thereof, may be administered as a suitable dosage form, as described herein, to treat, ameliorate, and/or reduce the severity of S-SCC cancer or one or more symptoms thereof in an S-SCC cancer subject. In some embodiments, an nNOS inhibitor composition comprising one or more antibodies selected from the group consisting of 37-2800 (from ThermoFisher), EP1855Y (from abeam), EPR 24351-6 (from abeam), and any combination thereof, may be administered to an S-SCC cancer subject to treat, ameliorate, and/or reduce the severity of S-SCC cancer or one or more symptoms thereof in the S-SCC cancer subject: (i) via a route selected from the group consisting of buccally, inhalation, intraarterially, intraarticularly, intracardially, intracerebroventricularly, intradermally, intramedullary, intramuscularly, intraperitoneally, intrathecally, intravenously, intraventricularly, nasally, ophthalmically, orally, rectally, sublabially, sublingually, subcutaneously, topically, and transdermally; and/or (ii) with a frequency selected from the group consisting of four times daily, three times daily, twice daily, once daily, once every two days, once every three days, once every four days, once every five days, once every six days, once a week, once every eight days, once every nine days, once every ten days, once every two weeks, once every three weeks, and once a month; and/or (iii) at a dosage (based on subject weight) selected from the group consisting of 0.1 μg/kg, 0.5 μg/kg, 1 μg/kg, 1.5 μg/kg, 2.0 μg/kg, 2.5 μg/kg, 3.0 μg/kg, 3.5 μg/kg, 4.0 μg/kg, 4.5 μg/kg, 5.0 μg/kg, 10 μg/kg, 15 μg/kg, 20 μg/kg, 25 μg/kg, 30 μg/kg, 35 μg/kg, 40 μg/kg, 45 μg/kg, 50 μg/kg, 55 μg/kg, 60 μg/kg, 65 μg/kg, 70 μg/kg, 75 μg/kg, 80 μg/kg, 85 μg/kg, 90 μg/kg, 95 μg/kg, 100 μg/kg, 150 μg/kg, 200 μg/kg, 250 μg/kg, 300 μg/kg, 350 μg/kg, 400 μg/kg, 450 μg/kg, 500 μg/kg, 550 μg/kg, 600 μg/kg, 650 μg/kg, 700 μg/kg, 750 μg/kg, 800 μg/kg, 850 μg/kg, 900 μg/kg, 1 mg/kg, 1.5 mg/kg, 2.0 mg/kg, 2.5 mg/kg, 3 mg/kg, 3.5 mg/kg, 4.0 mg/kg, 4.5 mg/kg, 5 mg/kg, 5.5 mg/kg, 6 mg/kg, 6.5 mg/kg, 7 mg/kg, 7.5 mg/kg, 8 mg/kg, 8.5 mg/kg, 9 mg/kg, 9.5 mg/kg, 10 mg/kg 10.5 mg/kg, 11 mg/kg, 11.5 mg/kg, 12 mg/kg, 12.5 mg/kg, 13 mg/kg, 13.5 mg/kg, 14 mg/kg, 14.5 mg/kg, 15 mg/kg, 16 mg/kg, 17 mg/kg, 18 mg/kg, 19 mg/kg, 20 mg/kg, 21 mg/kg, 22 mg/kg, 23 mg/kg, 24 mg/kg, 25 mg/kg, 30 mg/kg, 35 mg/kg, 40 mg/kg, 45 mg/kg, 50 mg/kg, 55 mg/kg, 60 mg/kg, 65 mg/kg, 70 mg/kg, 75 mg/kg, 80 mg/kg, 85 mg/kg, 90 mg/kg, 95 mg/kg, 100 mg/kg, 105 mg/kg, 110 mg/kg, 115 mg/kg, 120 mg/kg, 125 mg/kg, 130 mg/kg, 135 mg/kg, 140 mg/kg, 145 mg/kg, 150 mg/kg, 155 mg/kg, 160 mg/kg, 165 mg/kg, 170 mg/kg, 175 mg/kg, 180 mg/kg, 185 mg/kg, 190 mg/kg, 195 mg/kg, 200 mg/kg, and any fraction or integer in between any two of the preceding amounts.

In some embodiments, an nNOS inhibitor composition comprising one or more amino acid-based compounds, may be administered as a suitable dosage form, as described herein, to treat, ameliorate, and/or reduce the severity of S-SCC cancer or one or more symptoms thereof in an S-SCC cancer subject. In certain embodiments, an nNOS inhibitor composition comprising one or more amino acid-compounds that bind an active site of nNOS selected from the group consisting of Phenylalanine 589, Tryptophan 592, Tyrosine 593, Glutamate 597, Aspartate 602, Arginine 601, Methionine 341, Histidine 342, and Tyrosine711, may be administered as a suitable dosage form, as described herein, to treat, ameliorate, and/or reduce the severity of S-SCC cancer or one or more symptoms thereof in an S-SCC cancer subject. In some embodiments, an nNOS inhibitor composition comprising one or more amino acid-compounds that bind an active site of nNOS selected from the group consisting of Phenylalanine 589, Tryptophan 592, Tyrosine 593, Glutamate 597, Aspartate 602, Arginine 601, Methionine 341, Histidine 342, and Tyrosine711, may be administered to an S-SCC cancer subject to treat, ameliorate, and/or reduce the severity of S-SCC cancer or one or more symptoms thereof in the S-SCC cancer subject: (i) via a route selected from the group consisting of buccally, inhalation, intraarterially, intraarticularly, intracardially, intracerebroventricularly, intradermally, intramedullary, intramuscularly, intraperitoneally, intrathecally, intravenously, intraventricularly, nasally, ophthalmically, orally, rectally, sublabially, sublingually, subcutaneously, topically, and transdermally; and/or (ii) with a frequency selected from the group consisting of four times daily, three times daily, twice daily, once daily, once every two days, once every three days, once every four days, once every five days, once every six days, once a week, once every eight days, once every nine days, once every ten days, once every two weeks, once every three weeks, and once a month; and/or (iii) at a dosage (based on subject weight) selected from the group consisting of 0.1 μg/kg, 0.5 μg/kg, 1 μg/kg, 1.5 μg/kg, 2.0 μg/kg, 2.5 μg/kg, 3.0 μg/kg, 3.5 μg/kg, 4.0 μg/kg, 4.5 μg/kg, 5.0 μg/kg, 10 μg/kg, 15 μg/kg, 20 μg/kg, 25 μg/kg, 30 μg/kg, 35 μg/kg, 40 μg/kg, 45 μg/kg, 50 μg/kg, 55 μg/kg, 60 μg/kg, 65 μg/kg, 70 μg/kg, 75 μg/kg, 80 μg/kg, 85 μg/kg, 90 μg/kg, 95 μg/kg, 100 μg/kg, 150 μg/kg, 200 μg/kg, 250 μg/kg, 300 μg/kg, 350 μg/kg, 400 μg/kg, 450 μg/kg, 500 μg/kg, 550 μg/kg, 600 μg/kg, 650 μg/kg, 700 μg/kg, 750 μg/kg, 800 μg/kg, 850 μg/kg, 900 μg/kg, 1 mg/kg, 1.5 mg/kg, 2.0 mg/kg, 2.5 mg/kg, 3 mg/kg, 3.5 mg/kg, 4.0 mg/kg, 4.5 mg/kg, 5 mg/kg, 5.5 mg/kg, 6 mg/kg, 6.5 mg/kg, 7 mg/kg, 7.5 mg/kg, 8 mg/kg, 8.5 mg/kg, 9 mg/kg, 9.5 mg/kg, 10 mg/kg 10.5 mg/kg, 11 mg/kg, 11.5 mg/kg, 12 mg/kg, 12.5 mg/kg, 13 mg/kg, 13.5 mg/kg, 14 mg/kg, 14.5 mg/kg, 15 mg/kg, 16 mg/kg, 17 mg/kg, 18 mg/kg, 19 mg/kg, 20 mg/kg, 21 mg/kg, 22 mg/kg, 23 mg/kg, 24 mg/kg, 25 mg/kg, 30 mg/kg, 35 mg/kg, 40 mg/kg, 45 mg/kg, 50 mg/kg, 55 mg/kg, 60 mg/kg, 65 mg/kg, 70 mg/kg, 75 mg/kg, 80 mg/kg, 85 mg/kg, 90 mg/kg, 95 mg/kg, 100 mg/kg, 105 mg/kg, 110 mg/kg, 115 mg/kg, 120 mg/kg, 125 mg/kg, 130 mg/kg, 135 mg/kg, 140 mg/kg, 145 mg/kg, 150 mg/kg, 155 mg/kg, 160 mg/kg, 165 mg/kg, 170 mg/kg, 175 mg/kg, 180 mg/kg, 185 mg/kg, 190 mg/kg, 195 mg/kg, 200 mg/kg, and any fraction or integer in between any two of the preceding amounts.

In some embodiments, a s-SCC subject, as described herein, may be a female child or a male child. In certain embodiments, a s-SCC subject, as described herein, may be a female adult or a male adult.

In some embodiments, a s-SCC subject, as described herein, may have an abnormal expression of one or more antigens compared to an equivalent subject that does not have s-SCC. As used herein, the term “equivalent subject,” refers to a subject of the same sex (male/female) and age range (child/adult) of the s-SCC subject. For purposes of disclosure, the term “antigen,” when used with respect to abnormal expression, may define one or more cluster of differentiation (“CD”) antigens, such as, for example, CD15, CD44, CD70, CD133, as well as any CD antigen that has an abnormal expression in s-SCC that would be readily envisaged by one skilled in the art. In certain embodiments, abnormal expression of an antigen may define an abnormal expression of CD15, an abnormal expression of CD44, an abnormal expression of CD70, an abnormal expression of CD133, an abnormal expression of a CD antigen that has an abnormal expression in s-SCC that would be readily envisaged by one skilled in the art, and any combination thereof. In some embodiments, an “abnormal expression,” can refer to a reduced expression of an antigen, as described herein. In certain embodiments, an “abnormal expression,” can refer to an overexpression of an antigen, as described herein. Whether “abnormal expression,” refers to a reduced expression of an antigen or an overexpression of an antigen would be immediately envisaged by the skilled artisan when viewing the term in the context of the particular antigen disclosed and s-SCC. For example, in the context of CD10 and s-SCC, one skilled in the art would immediately envisage that “abnormal expression” refers to an overexpression of CD10. Treatment, amelioration, and/or reduction in the severity of s-SCC or one or more symptoms thereof may be characterized by a restoration to a normal level of expression of one or more antigens from their abnormal state, as described herein. For example, in the context of CD10 and s-SCC, treatment, amelioration, and/or reduction in the severity of s-SCC or one or more symptoms thereof may be characterized by a reduced expression of CD10.

In certain embodiments, a s-SCC subject, as described herein, may have an abnormal expression of one or more proteins compared to an equivalent subject that does not have s-SCC. For purposes of disclosure, the term “protein,” when used with respect to abnormal expression, may define one or more proteins and/or enzymes, such as, for example, ALDH1A3, mTOR, p-mTOR, TSC-2, AKT, pAKT, RPS6, p-RPS6, Syp, VEGF, PIGF, as well as any protein or enzyme that has an abnormal expression in s-SCC that would be readily envisaged by one skilled in the art. In certain embodiments, abnormal expression of a protein may define an abnormal expression of ALDH1A3, an abnormal expression of mTOR, an abnormal expression of p-mTOR, an abnormal expression of TSC-2, an abnormal expression of AKT, an abnormal expression of pAKT, an abnormal expression of RPS6, an abnormal expression of p-RPS6, an abnormal expression of Syp, an abnormal expression of VEGF, an abnormal expression of PIGF, an abnormal expression of a protein or enzyme that has an abnormal expression in s-SCC that would be readily envisaged by one skilled in the art, and any combination thereof. In some embodiments, an “abnormal expression,” can refer to a reduced expression of a protein, as described herein. In certain embodiments, an “abnormal expression,” can refer to an overexpression of a protein, as described herein. Whether “abnormal expression,” refers to a reduced expression of a protein or an overexpression of a protein would be immediately envisaged by the skilled artisan when viewing the term in the context of the particular protein disclosed and s-SCC. For example, in the context of s-SCC, one skilled in the art would immediately envisage that “abnormal expression” refers to an overexpression of mTOR. Treatment, amelioration, and/or reduction in the severity of s-SCC or one or more symptoms thereof may be characterized by a restoration to a normal level of expression of one or more proteins from their abnormal state, as described herein. For example, in the context of s-SCC treatment, amelioration, and/or reduction in the severity of s-SCC or one or more symptoms thereof may be characterized by a reduced expression of mTOR.

In certain embodiments, a s-SCC subject, as described herein, may have an increased expression of one or more NOS isoforms compared to an equivalent subject that does not have s-SCC. For purposes of disclosure, the term “NOS isoform,” refers to nNOS, iNOS, and/or eNOS. In certain embodiments, a s-SCC subject, as described herein, may have an increased expression of nNOS compared to an equivalent subject that does not have s-SCC. In some embodiments, a s-SCC subject, as described herein, may have an increased expression of iNOS compared to an equivalent subject that does not have s-SCC. In certain embodiments, a s-SCC subject, as described herein, may have an increased expression of eNOS compared to an equivalent subject that does not have s-SCC. In some embodiments, a s-SCC subject, as described herein, may have an increased expression of nNOS and iNOS compared to an equivalent subject that does not have s-SCC. In certain embodiments, a s-SCC subject, as described herein, may have an increased expression of nNOS and eNOS compared to an equivalent subject that does not have s-SCC. In some embodiments, a s-SCC subject, as described herein, may have an increased expression of iNOS and eNOS compared to an equivalent subject that does not have s-SCC. In certain embodiments, a s-SCC subject, as described herein, may have an increased expression of nNOS, iNOS, and eNOS compared to an equivalent subject that does not have s-SCC. Whether a “NOS isoform,” has an increased expression would be immediately envisaged by the skilled artisan when viewing the term in the context of the particular NOS isoform disclosed and s-SCC. For example, in the context of s-SCC, one skilled in the art would immediately envisage that iNOS has an increased expression. Treatment, amelioration, and/or reduction in the severity of s-SCC or one or more symptoms thereof may be characterized by a reduced expression of one or more NOS isoforms from their increased state, as described herein. For example, in the context of s-SCC, treatment, amelioration, and/or reduction in the severity of s-SCC or one or more symptoms thereof may be characterized by a reduced expression of iNOS. For purposes of disclosure, reduction/inhibition of the expression of one or more NOS isoforms may be characterized by a reduction in expression of one or more NOS isoforms by about at least 10%, about at least 20%, about at least 30%, or about at least 40%. In some embodiments, reduction/inhibition of the expression of one or more NOS isoforms may further be characterized by a lack reduction of other, non-implicated NOS isoforms, such as, for example, no more than about 10%, no more than about 5%, no more than about 2%, or no more than about 1% reduction/inhibition of the expression of non-implicated NOS isoforms. As used herein, the term “non-implicated NOS isoforms,” refers to NOS isoforms that do not have an increased expression in s-SCC. By way of example, in the context of s-SCC if both iNOS has an increased expression, nNOS and/or eNOS would define a “non-implicated” NOS isoform. Whether a NOS isoform is a “non-implicated NOS isoform,” would be immediately envisaged by the skilled artisan when viewing the term in the context of the particular NOS isoform disclosed and s-SCC.

In some embodiments, a s-SCC subject, as described herein, may have an elevated level of one or more NO-indicators compared to an equivalent subject that does not have s-SCC. As used herein, the term “NO-indicator,” can refer to 3-nitrotyrosine (3-Ntyr), NADPH-diaphorase, total nitrite concentration, total nitrate concentration, total nitrite and nitrate concentration, as well as any NO-indicator that has an elevated level in s-SCC that would be readily envisaged by one skilled in the art. For example, in the context of s-SCC, one skilled in the art would immediately envisage that 3-Ntyr can be present at an elevated level. Treatment, amelioration, and/or reduction in the severity of s-SCC or one or more symptoms thereof may be characterized by a reduction in the level of one or more NO-indicators. For example, in the context of s-SCC, treatment, amelioration, and/or reduction in the severity of s-SCC or one or more symptoms thereof may be characterized by a reduction in the level of 3-Ntyr.

In certain embodiments, a NOS inhibitor composition, as described herein, may be administered as a suitable dosage form, as described herein, to treat, ameliorate, and/or reduce the severity of s-SCC or one or more symptoms thereof, as an alternative to a conventional therapy for treating, ameliorating, and/or reducing the severity of s-SCC or one or more symptoms thereof. For purposes of disclosure, a “conventional therapy,” as described herein, may refer to a treatment that would be recommended by one skilled in the art to treat s-SCC, and may define one or more of radiation, chemotherapy, immunotherapy, cell-based therapy, gene therapy, and the like—one skilled in the art would immediately envisage a “conventional therapy” based on the particular s-SCC disclosed, as well as characteristics of a s-SCC subject, such as, for example, the subject's sex, age, stage of the s-SCC, genotype of the s-SCC, and the like. Treatment, amelioration, and/or reduction in the severity of s-SCC or one or more symptoms thereof may be characterized by an improved outcome compared to a conventional therapy, as described herein. For purposes of the disclosure, an “improved outcome” may define any one of the following: (i) a larger reduction in tumor volume and/or mass compared to a subject administered an equivalent dose of a conventional therapy; (ii) an improvement in the expected rate of survival of a subject, at, for example, 1 month, 3 months, 6 months, 9 months, 1 year, 2 years, 3 years, 5 years, 10 years, or more, compared to the expected rate of survival of a subject administered an equivalent dose of a conventional therapy; (iii) a reduction in the prevalence of side effects resulting from the NOS inhibitor composition compared to a subject administered an equivalent dose of a conventional therapy; (iv) a reduction in the severity of side effects resulting from the NOS inhibitor composition compared to a subject administered an equivalent dose of a conventional therapy; (v) a reduction in the dosage required to achieve an equivalent reduction in tumor volume compared to a subject administered a conventional therapy; (vi) a reduction in the dosage required to achieve an equivalent expected rate of survival of a subject compared to a subject administered a conventional therapy; (vii) a reduction in the likelihood of a recurrence of the s-SCC occurring in a subject compared to a subject administered an equivalent dose of a conventional therapy; (viii) a reduction in the likelihood of a metastasis occurring in a subject compared to a subject administered an equivalent dose of a conventional therapy; (ix) a reduction in the time required to be at a medical facility for treatment compared to a subject administered an equivalent dose of a conventional therapy; (x) a reduction in the cost associated with treatment compared to a subject administered an equivalent dose of a conventional therapy; as well as any other improved outcome not expressly recited that would be readily envisaged by one skilled in the art.

In some embodiments, a NOS inhibitor composition, as described herein, may be administered as a suitable dosage form, as described herein, to treat, ameliorate, and/or reduce the severity of s-SCC or one or more symptoms thereof, wherein the NOS inhibitor composition comprises a first chemical NOS inhibitor and a second chemical NOS inhibitor. In certain embodiments, the first chemical NOS inhibitor may be administered concurrently with the second chemical NOS inhibitor. In some embodiments, the first chemical NOS inhibitor may be administered prior to the second chemical NOS inhibitor. In certain embodiments, the first chemical NOS inhibitor may be administered subsequently to the second chemical NOS inhibitor. Treatment, amelioration, and/or reduction in the severity of s-SCC or one or more symptoms thereof may be characterized by an improved outcome compared an alternative treatment. As used herein, the term “alternative treatment,” may define one or more of a conventional therapy, as described herein, a NOS inhibitor composition comprising a single chemical NOS inhibitor, as described herein, and a NOS inhibitor composition comprising a single biological NOS inhibitor, as described herein. For purposes of the disclosure, an “improved outcome” may define any one of the following: (i) a larger reduction in tumor volume and/or mass compared an alternative treatment; (ii) an improvement in the expected rate of survival of a subject, at, for example, 1 month, 3 months, 6 months, 9 months, 1 year, 2 years, 3 years, 5 years, 10 years, or more, compared to the expected rate of survival of a subject administered an alternative treatment; (iii) a reduction in the prevalence of side effects resulting from the NOS inhibitor composition comprising at least two chemical NOS inhibitors compared to a subject administered an alternative treatment; (iv) a reduction in the severity of side effects resulting from the NOS inhibitor composition comprising at least two chemical NOS inhibitors compared to a subject administered an alternative treatment; (v) a reduction in the dosage required to achieve an equivalent reduction in tumor volume compared to a subject administered an alternative treatment; (vi) a reduction in the dosage required to achieve an equivalent expected rate of survival of a subject compared to a subject administered an alternative treatment; (vii) a reduction in the likelihood of a recurrence of the s-SCC in a subject compared to a subject administered an alternative treatment; (viii) a reduction in the likelihood of a metastasis occurring in a subject compared to a subject administered an alternative treatment; (ix) a reduction in the time required to be at a medical facility for treatment compared to a subject administered an alternative treatment; (x) a reduction in the cost associated with treatment compared to a subject administered an alternative treatment; as well as any other improved outcome not expressly recited that would be readily envisaged by one skilled in the art.

In certain embodiments, a NOS inhibitor composition, as described herein, may be used to treat, ameliorate, and/or reduce the severity of s-SCC or one or more symptoms thereof, wherein the NOS inhibitor composition comprises at least one chemical NOS inhibitor and at least one biological NOS inhibitor. In some embodiments, the at least one chemical NOS inhibitor may be administered concurrently with the at least one biological NOS inhibitor. In certain embodiments, the at least one chemical NOS inhibitor may be administered prior to the at least one biological NOS inhibitor. In some embodiments, the at least one chemical NOS inhibitor may be administered subsequently to the at least one biological NOS inhibitor. Treatment, amelioration, and/or reduction in the severity of s-SCC or one or more symptoms thereof may be characterized by an improved outcome compared an alternative treatment. As used herein, the term “alternative treatment,” may define one or more of a conventional therapy, as described herein, a NOS inhibitor composition comprising a single chemical NOS inhibitor, as described herein, and a NOS inhibitor composition comprising a single biological NOS inhibitor, as described herein. For purposes of the disclosure, an “improved outcome” may define any one of the following: (i) a larger reduction in tumor volume and/or mass compared an alternative treatment; (ii) an improvement in the expected rate of survival of a subject, at, for example, 1 month, 3 months, 6 months, 9 months, 1 year, 2 years, 3 years, 5 years, 10 years, or more, compared to the expected rate of survival of a subject administered an alternative treatment; (iii) a reduction in the prevalence of side effects resulting from the NOS inhibitor composition comprising at least one chemical NOS inhibitor and at least one biological NOS inhibitor compared to a subject administered an alternative treatment; (iv) a reduction in the severity of side effects resulting from the NOS inhibitor composition comprising at least one chemical NOS inhibitor and at least one biological NOS inhibitor compared to a subject administered an alternative treatment; (v) a reduction in the dosage required to achieve an equivalent reduction in tumor volume compared to a subject administered an alternative treatment; (vi) a reduction in the dosage required to achieve an equivalent expected rate of survival of a subject compared to a subject administered an alternative treatment; (vii) a reduction in the likelihood of a recurrence of the s-SCC in a subject compared to a subject administered an alternative treatment; (viii) a reduction in the likelihood of a metastasis occurring in a subject compared to a subject administered an alternative treatment; (ix) a reduction in the time required to be at a medical facility for treatment compared to a subject administered an alternative treatment; (x) a reduction in the cost associated with treatment compared to a subject administered an alternative treatment; as well as any other improved outcome not expressly recited that would be readily envisaged by one skilled in the art.

In some embodiments, a NOS inhibitor composition, as described herein, may be combined with a conventional therapy, as described herein, to treat, ameliorate, and/or reduce the severity of s-SCC or one or more symptoms thereof. In certain embodiments, the NOS inhibitor composition may be administered concurrently with the conventional therapy. In some embodiments, the NOS inhibitor composition may be administered prior to the conventional therapy. In certain embodiments, the NOS inhibitor composition may be administered subsequently to the conventional therapy. Treatment, amelioration, and/or reduction in the severity of s-SCC or one or more symptoms thereof may be characterized by an improved outcome compared an alternative treatment. As used herein, the term “alternative treatment,” may define one or more of a conventional therapy, as described herein, a NOS inhibitor composition comprising a single chemical NOS inhibitor, as described herein, and a NOS inhibitor composition comprising a single biological NOS inhibitor, as described herein. For purposes of the disclosure, an “improved outcome” may define any one of the following: (i) a larger reduction in tumor volume and/or mass compared an alternative treatment; (ii) an improvement in the expected rate of survival of a subject, at, for example, 1 month, 3 months, 6 months, 9 months, 1 year, 2 years, 3 years, 5 years, 10 years, or more, compared to the expected rate of survival of a subject administered an alternative treatment; (iii) a reduction in the prevalence of side effects resulting from the NOS inhibitor composition and conventional therapy compared to a subject administered an alternative treatment; (iv) a reduction in the severity of side effects resulting from the NOS inhibitor composition and conventional therapy compared to a subject administered an alternative treatment; (v) a reduction in the dosage required to achieve an equivalent reduction in tumor volume compared to a subject administered an alternative treatment; (vi) a reduction in the dosage required to achieve an equivalent expected rate of survival of a subject compared to a subject administered an alternative treatment; (vii) a reduction in the likelihood of a recurrence of the s-SCC in a subject compared to a subject administered an alternative treatment; (viii) a reduction in the likelihood of a metastasis occurring in a subject compared to a subject administered an alternative treatment; (ix) a reduction in the time required to be at a medical facility for treatment compared to a subject administered an alternative treatment; (x) a reduction in the cost associated with treatment compared to a subject administered an alternative treatment; as well as any other improved outcome not expressly recited that would be readily envisaged by one skilled in the art.

In some embodiments, a “conventional therapy,” as that term is used with respect to combined administration with a NOS inhibitor composition, may refer to a conventional chemotherapy. For purposes of the disclosure, a “conventional chemotherapy,” as described herein may be a compound selected from the group consisting of abemaciclib, abiraterone acetate, acalabrutinib, adagrasib, afatinib dimaleate, albumin-bound paclitaxel, albumin-bound rapamycin (Sirolimus Protein-Bound Particles), alectinib, alpelisib, amifostine, aminolevulinic acid hydrochloride, anastrozole, apalutamide, aprepitant, arsenic trioxide, asciminib hydrochloride, avapritinib, axitinib, azacytidine, belinostat, belzutifan, bendamustine hydrochloride, bexarotene, bicalutamide, binimetinib, bleomycin, bleomycin sulfate, bortezomib, bosutinib, brigatinib, busulfan, cabazitaxel, cabozantinib-S-malate, capecitabine, capmatinib hydrochloride, carboplatin, carfilzomib, carmustine, cedazuridine, ceritinib, chlorambucil, cisplatin, cladribine, clofarabine, cobimetinib fumarate, copanlisib hydrochloride, crizotinib, cyclophosphamide, cytarabine, dabrafenib mesylate, dacarbazine, dacomitinib, dactinomycin, darolutamide, dasatinib, daunorubicin hydrochloride, decitabine, defibrotide sodium, degarelix, dexamethasone, dexrazoxane hydrochloride, docetaxel, doxorubicin hydrochloride, duvelisib, elacestrant dihydrochloride, eltrombopag olamine, enasidenib mesylate, encorafenib, entrectinib, enzalutamide, epirubicin hydrochloride, erdafitinib, eribulin mesylate, erlotinib hydrochloride, etoposide, etoposide phosphate, everolimus, exemestane, fedratinib hydrochloride, fludarabine phosphate, fluorouracil, flutamide, folinic acid, fostamatinib disodium, fulvestrant, futibatinib, gefitinib, gemcitabine hydrochloride, gilteritinib fumarate, glasdegib maleate, goserelin acetate, granisetron, granisetron hydrochloride, hydroxyurea, ibrutinib, idarubicin hydrochloride, idelalisib, ifosfamide, imatinib mesylate, imiquimod, infigratinib phosphate, iobenguane I-131, irinotecan hydrochloride, ivosidenib, ixabepilone, ixazomib citrate, lanreotide acetate, lapatinib ditosylate, larotrectinib sulfate, lenalidomide, lenvatinib mesylate, letrozole, leucovorin calcium, leuprolide acetate, liposomal cytarabine, liposomal doxorubicin hydrochloride, liposomal irinotecan hydrochloride, lomustine, lorlatinib, lurbinectedin, Lu-177 dotatate, Lu-177 vipivotide tetraxetan, mechlorethamine hydrochloride, megestrol acetate, melphalan, melphalan hydrochloride, mercaptopurine, mesna, methotrexate, methotrexate sodium, methylnaltrexone bromide, midostaurin, mitomycin, mitoxantrone hydrochloride, mobocertinib succinate, nelarabine, neratinib maleate, netupitant hydrochloride, nilutamide, nilotinib, niraparib tosylate monohydrate, olaparib, olutasidenib, omacetaxine mepesuccinate, ondansetron hydrochloride, osimertinib mesylate, oxaliplatin, paclitaxel, pacritinib citrate, palbociclib, palonosetron hydrochloride, pamidronate disodium, pazopanib hydrochloride, pemetrexed disodium, pemigatinib, pexidartinib hydrochloride, pirtobrutinib, plerixafor, pomalidomide, ponatinib hydrochloride, pralatrexate, pralsetinib, prednisone, procarbazine hydrochloride, propranolol hydrochloride, Ra-223 dichloride, raloxifene hydrochloride, regorafenib, relugolix, ribociclib, ripretinib, rolapitant hydrochloride, romidepsin, rucaparib camsylate, ruxolitinib phosphate, selinexor, selpercatinib, selumetinib sulfate, sonidegib, sorafenib tosylate, sotorasib, sunitinib malate, talazoparib tosylate, talc, tamoxifen citrate, tazemetostat hydrobromide, temozolomide, temsirolimus, tepotinib hydrochloride, thalidomide, thioguanine, thiotepa, tipiracil hydrochloride, tivozanib hydrochloride, topotecan hydrochloride, toremifene, trabectedin, trametinib dimethyl sulfoxide, tretinoin, trifluridine hydrochloride, tucatinib, uridine triacetate, valrubicin, vandetanib, vemurafenib, venetoclax, vinblastine sulfate, vincristine sulfate, vinorelbine tartrate, vismodegib, vorinostat, zanubrutinib, zoledronic acid, and any combination thereof. One skilled in the art would immediately envisage an appropriate “conventional chemotherapy” based on the particular s-SCC disclosed, as well as characteristics of a m s-SCC subject, such as, for example, the subject's sex, age, stage of the s-SCC, genotype of the s-SCC, and the like.

In certain embodiments, a “conventional therapy,” as that term is used with respect to combined administration with a NOS inhibitor composition, may refer to a conventional immunotherapy. For purposes of the disclosure, a “conventional immunotherapy,” as described herein may be an immunotherapy, cell-based therapy, and/or gene-based therapy selected from the group consisting of ado-trastuzumab emtansine, aldesleukin, alemtuzumab, amivantamab-vmjw, asparaginase Erwinia chrysanthemi, asparaginase Erwinia chrysanthemi (recombinant)-rywn, atezolizumab, avelumab, axicabtagene ciloleucel, bevacizumab, blinatumomab, brentuximab vedotin, brexucabtagene autoleucel, calaspargase pegol-mknl, camrelizumab, caplacizumab-yhdp, cemiplimab-rwlc, cetuximab, ciltacabtagene autoleucel, daratumumab, daratumumab and hyaluronidase-fihj, darbepoetin alfa, denileukin diftitox, denosumab, dinutuximab, dostarlimab-gxly, durvalumab, elotuzumab, emapalumab-lzsg, enfortumab vedotin-ejfv, epoetin alfa, fam-trastuzumab deruxtecan-nxki, filgrastim, gemtuzumab ozogamicin, glucarpidase, ibritumomab tiuxetan, idecabtagene vicleucel, inotuzumab ozogamicin, ipilimumab, isatuximab-irfc, lisocabtagene maraleucel, loncastuximab tesirine-lpyl, luspatercept-aamt, margetuximab-cmkb, mirvetuximab soravtansine-gynx, mogamulizumab-kpkc, mosunetuzumab-axgb, moxetumomab pasudotox-tdfk, nadofaragene firadenovec-vncg, naxitamab-gqgk, necitumumab, nivolumab, nivolumab and relatlimab-rmbw, obinutuzumab, ofatumumab, palifermin, panitumumab, pegaspargase, pegfilgrastim, pembrolizumab, pertuzumab, pertuzumab, trastuzumab, and hyaluronidase-zzxf, polatuzumab vedotin-piiq, ramucirumab, rasburicase, ravulizumab-cwvz, recombinant HPV bivalent vaccine, recombinant HPV nonavalent vaccine, recombinant HPV quadrivalent vaccine, recombinant interferon alfa-2b, rituximab, rituximab and hyaluronidase human, romiplostim, ropeginterferon alfa-2b-njft, sacituzumab govitecan-hziy, siltuximab, sintilimab, sipuleucel-T, tafasitamab-cxix, tagraxofusp-erzs, talimogene laherparepvec, tebentafusp-tebn, teclistamab-cqyv, tisagenlecleucel, tislelizumab, tisotumab vedotin-tftv, tocilizumab, toripalimab, trastuzumab, trastuzumab and hyaluronidase-oysk, tremelimumab-actl, ziv-aflibercept, and any combination thereof. One skilled in the art would immediately envisage an appropriate “conventional immunotherapy” based on the particular s-SCC disclosed, as well as characteristics of a s-SCC subject, such as, for example, the subject's sex, age, stage of the s-SCC, genotype of the s-SCC, and the like.

In certain embodiments, a NOS inhibitor composition, as described herein, may be administered as a suitable dosage form, as described herein, prior to, or alternatively, following a surgery to remove or resect s-SCC.

In some embodiments, a NOS inhibitor composition, as described herein, may be administered as a suitable dosage form, as described herein, to restore and/or maintain healthy and/or optimal expression levels of one or more antigens, as described herein, such as, for example, CD15, CD44, CD70, CD133, as well as any CD antigen that has an abnormal expression in s-SCC that would be readily envisaged by one skilled in the art, in a subject. In certain embodiments, the subject may have s-SCC or one or more symptoms thereof. As used herein, the terms “healthy” and “optimal” refer to expression levels of one or more antigens that would be typical of an equivalent subject that does not have s-SCC.

In certain embodiments, a NOS inhibitor composition, as described herein, may be administered as a suitable dosage form, as described herein, to restore and/or maintain healthy and/or optimal expression levels of one or more proteins, as described herein, such as, for example, ALDH1A3, mTOR, p-mTOR, TSC-2, AKT, pAKT, RPS6, p-RPS6, Syp, VEGF, PIGF, as well as any protein or enzyme that has an abnormal expression in s-SCC that would be readily envisaged by one skilled in the art, in a subject. In certain embodiments, the subject may have s-SCC or one or more symptoms thereof. As used herein, the terms “healthy” and “optimal” refer to expression levels of one or more proteins that would be typical of an equivalent subject that does not have s-SCC.

In some embodiments, a NOS inhibitor composition, as described herein, may be administered as a suitable dosage form, as described herein, to restore and/or maintain healthy and/or optimal expression levels of one or more NOS isoforms, as described herein, such as, for example, nNOS, iNOS, and eNOS, in a subject. In certain embodiments, the subject may have s-SCC or one or more symptoms thereof. As used herein, the terms “healthy” and “optimal” refer to expression levels of one or more NOS isoforms that would be typical of an equivalent subject that does not have s-SCC.

In certain embodiments, a NOS inhibitor composition, as described herein, may be administered as a suitable dosage form, as described herein, to restore and/or maintain healthy and/or optimal levels of one or more NO-indicators, as described herein, such as, for example, 3-Ntyr, NADPH-diaphorase, total nitrite concentration, total nitrate concentration, total nitrite and nitrate concentration, as well as any NO-indicator that has an elevated level in s-SCC that would be readily envisaged by one skilled in the art, in a subject. In certain embodiments, the subject may have s-SCC or one or more symptoms thereof. As used herein, the terms “healthy” and “optimal” refer to levels of one or more NO-indicators that would be typical of an equivalent subject that does not have s-SCC.

The compositions disclosed herein may be formulated to comprise a NOS inhibitor in a neutral or salt form. For purposes of this disclosure, “salts” may include, but is not limited to salts derived from inorganic acids like hydrochloric, hydrobromic, nitric, carbonic, monohydrogencarbonic, phosphoric, monohydrogenphosphoric, dihydrogenphosphoric, sulfuric, monohydrogensulfuric, hydriodic, or phosphorous acids, and the like, as well as the salts derived from relatively nontoxic organic acids like acetic acid, propionic acid, isobutyric acid, maleic acid, malonic acid, benzoic acid, succinic acid, suberic acid, fumaric acid, lactic acid, mandelic acid, phthalic acid, benzenesulfonic acid, p-tolylsulfonic acid, citric acid, tartaric acid, methanesulfonic acid, and the like. Also included are salts of amino acids such as arginate and the like, and salts of organic acids like glucuronic or galacturonic acids, and the like. (see, for example, Berge et al., J. Pharm. Sci. 1977; 66: 1-19, which is hereby incorporated by reference in its entirety). Further included are salts any suitable elemental cation, such as potassium, calcium, and the like, any suitable elemental anion, such as chlorine, bromine, and the like, as well as any pharmaceutically acceptable salt, as would be understood by one of ordinary skill in the art. Depending on the stoichiometric proportions between the charged group(s) in the compound and the counter-ion in the salt, salts disclosed herein can be either mono-addition salts or poly-addition salts. As used herein, the term “mono-addition salt”, refers to a salt in which the stoichiometric ratio between the counter-ion and charged form of the compound is 1:1, such that the addition salt includes one molar equivalent of the counter-ion per one molar equivalent of the compound. As used herein, the term “poly-addition salt” refers to a salt in which the stoichiometric ratio between the counter-ion and the charged form of the compound is greater than 1:1 and is, for example, 2:1, 3:1, 4:1 and so on, such that the addition salt includes two or more molar equivalents of the counter-ion per one molar equivalent of the compound.

As used herein, the term “pharmaceutically acceptable solvent” can refer to water or aqueous buffer solutions that are physiologically compatible, or aqueous solutions containing organic solvents that are physiologically compatible. A non-comprehensive list of pharmaceutically acceptable solvents is provided in U.S. Department of Health & Human Services, Food & Drug Administration, “Guidance for Industry: Q3C Impurities: Residual Solvents,” December 1997 or its current issue.

As used herein, the term “excipient” refers to any compound that is part of a formulation that is not an active ingredient, i.e., one that has no relevant biological activity, and which is added to the formulation to provide specific characteristics to the dosage form, including by way of example, providing protection to the active ingredient from chemical degradation, facilitating release of a tablet or caplet from equipment in which it is formed, and so forth.

The term “pharmaceutical formulation”, “formulation”, “composition,” and the like can refer to preparations that are in such a form as to permit the biological activity of the active ingredients to be effective, and therefore may be administered to a subject for therapeutic use along with pharmaceutical use. The meaning of these terms will be clear to the skilled artisan based upon the context in which they are used.

A “therapeutically effective amount,” as used herein, includes within its meaning, a non-toxic but sufficient amount of a compound active ingredient or composition comprising the same for use in the embodiments disclosed herein to provide the desired therapeutic effect. Similarly, “an amount effective to” or “an effective amount,” as used herein, includes within its meaning, a non-toxic but sufficient amount of a compound active ingredient or composition comprising the same to provide the desired effect. A “therapeutically effective amount” or an “effective amount” includes amounts of compounds that would not be achievable through a standard or natural diet, but requires supplementation and dosing, as described herein, to achieve specific, non-natural outcomes as set forth herein, along with expanded utilization of any compounds originating from or derived from natural sources. The exact amount of the active ingredient required may depend on factors such as the species being treated, the age and general condition of the subject, the severity of the condition being treated, the particular composition being administered, the weight of the subject, the mode of administration, and so forth. Thus, it may not always be possible to specify an exact “effective amount.” However, for any given case, an appropriate “effective amount” would be immediately envisaged by the skilled artisan in view of the context of the usage of the term “effective amount” and the disclosure set forth herein.

As used herein, the term “bioavailability” refers to the amount of a substance that is absorbed by a subject and ultimately available for biological activity in a subject's tissue and cells.

As used herein, “identifying,” refers to detecting or selecting a subject from a population of potential subjects, for example, to establish that a particular subject possesses certain properties or characteristics. “Identifying” may include, for example, self-identification, self-diagnosis, and diagnosis by a medical professional.

As used herein, the terms “preventing”, “treating”, “treatment,” “alleviating,” “ameliorating,” and the like are used herein to generally refer to obtaining a desired pharmacological and/or physiological effect, the scopes and meanings of which will be clear to the skilled artisan based upon the context in which these terms are used. The effect may be prophylactic in terms of preventing or partially preventing a disease, symptom, or condition thereof and/or may be therapeutic in terms of a partial or complete cure of a disease, condition, symptom, or adverse effect attributed to the disease. The term “treatment” as used herein encompasses any treatment of a disease in a mammal, particularly a human, and includes: (a) preventing the disease from occurring in a subject that may be predisposed to the disease but has not yet been diagnosed as having it; (b) inhibiting the disease or arresting its development; or (c) relieving the disease, causing regression of the disease and/or its symptoms, conditions, and co-morbidities. The scope and meaning of “preventing,” “treating,” “treatment,” “alleviating,” “ameliorating,” and “maintaining healthy levels of” would be immediately envisaged by the skilled artisan when viewing the term in the context of the disclosure and the claims.

As provided herein, the disclosure of a “ratio” of compounds and compositions corresponds to a ratio provided in terms of mass of the components present in the ratio.

When used in this disclosure, the phrase “consisting essentially of” is meant including any elements listed after the phrase and limited to other elements that do not interfere with or contribute to the activity or action specified in the disclosure for the listed elements. Thus, the phrase “consisting essentially of” indicates that the listed elements are required or mandatory, but that other elements are optional and can or cannot be present depending upon whether or not they affect the activity or action of the listed elements. For example, the use of a composition “consisting essentially of a composition” for the treatment of a particular disease or disorder, or the maintenance of a healthy condition, would exclude other ingredients that would materially alter the intended outcome of the composition.

As used herein, a composition that “substantially” comprises a compound means that the composition contains more than about 80% by weight, more preferably more than about 90% by weight, even more preferably more than about 95% by weight, and most preferably more than about 98% by weight of the compound.

To provide a more concise description, some of the quantitative expressions given herein are not qualified with the term “about.” It is understood that whether the term “about” is used explicitly or not, every quantity given herein is meant to refer to the actual given value, and it is also meant to refer to the approximation to such given value that would reasonably be inferred based on the ordinary skill in the art, including approximations due to the experimental and/or measurement conditions for such given value.

In addition, the appropriate dosage of the compositions can depend, for example, on the condition to be treated, the severity and course of the condition, whether the composition is administered for preventive or therapeutic purposes, previous therapy, the patient's clinical history and response to the composition, the type of composition used, and the discretion of the attending physician. The composition can be suitably administered to the patient at one time or over a series of treatments and may be administered to the patient at any time from diagnosis onwards. The composition may be administered as the sole treatment or in conjunction with other drugs or therapies useful in treating the condition in question.

The following are provided for the illustration of exemplary embodiments of the present disclosure.

A composition comprising an amount of at least one chemical NOS inhibitor.

A composition comprising an amount of at least one chemical NOS inhibitor, wherein the at least one chemical NOS inhibitor is selected from the group consisting of 7-nitroindazole (“7-NI”), a salt of 7-NI, 3-bromo-7-nitroindazole (“3-Br-7-NI”), a salt of 3-Br-7-NI, N^{ω}-propyl-L-arginine (“L-NPA”), L-NPA hydrochloride, N⁵-(1-iminoethyl)-L-ornithine (“L-NIO”), L-NIO dihydrochloride, N-[4-[2-[[(3-chlorophenyl)methyl]amino]ethyl]phenyl]-2-thiophenecarboximidamide (“ARL-17477”), ARL-17477 dihydrochloride, N^{ω}-nitro-L-arginine (“L-NNA”), N^{ω}-nitro-L-arginine methyl ester (“L-NAME”), L-NAME hydrochloride, N^{ω}-methyl-L-arginine (“L-NMMA”), L-NMMA acetate, S-[2-[(1-iminoethyl)amino]ethyl]-L-homocysteine (“GW-274150”), GW-274150 phosphate, N⁶-(1-iminoethyl)-L-lysine (“L-NIL”), L-NIL hydrochloride, N-[[3-(aminomethyl)phenyl]methyl]-ethanimidamide (“1400W”), 1400W dihydrochloride, 2,4-disulfophenyl-N-tert-butyl nitrone (“OKN-007”), OKN-007 sodium, 2-[2-(4-methoxypyridin-2-yl)ethyl]-1H-imidazo[4,5-b]pyridine (“BYK-191023”), BYK-191023 dihydrochloride, N-{3-[(ethanimidoylamino)methyl]benzyl}-L-prolinamide (“CM544”), CM544 dihydrochloride, and any combination thereof.

A composition comprising an amount of at least one chemical NOS inhibitor, wherein the at least one chemical NOS inhibitor comprises 7-NI and/or a salt of 7-NI.

A composition comprising an amount of at least one chemical NOS inhibitor, wherein the at least one chemical NOS inhibitor comprises 3-Br-7-NI and/or a salt of 3-Br-7-NI.

A composition comprising an amount of at least one chemical NOS inhibitor, wherein the at least one chemical NOS inhibitor comprises L-NPA and/or L-NPA hydrochloride.

A composition comprising an amount of at least one chemical NOS inhibitor, wherein the at least one chemical NOS inhibitor comprises L-NIO and/or L-NIO dihydrochloride.

A composition comprising an amount of at least one chemical NOS inhibitor, wherein the at least one chemical NOS inhibitor comprises ARL-17477 and/or ARL-17477 dihydrochloride.

A composition comprising an amount of at least one chemical NOS inhibitor, wherein the at least one chemical NOS inhibitor comprises L-NNA and/or a salt of L-NNA.

A composition comprising an amount of at least one chemical NOS inhibitor, wherein the at least one chemical NOS inhibitor comprises L-NAME and/or L-NAME hydrochloride.

A composition comprising an amount of at least one chemical NOS inhibitor, wherein the at least one chemical NOS inhibitor comprises L-NMMA and/or L-NMMA acetate.

A composition comprising an amount of at least one chemical NOS inhibitor, wherein the at least one chemical NOS inhibitor comprises GW-274150 and/or GW-274150 phosphate.

A composition comprising an amount of at least one chemical NOS inhibitor, wherein the at least one chemical NOS inhibitor comprises L-NIL and/or L-NIL hydrochloride.

A composition comprising an amount of at least one chemical NOS inhibitor, wherein the at least one chemical NOS inhibitor comprises L-NAME and/or L-NAME hydrochloride.

A composition comprising an amount of at least one chemical NOS inhibitor, wherein the at least one chemical NOS inhibitor comprises 1400W and/or 1400W dihydrochloride.

A composition comprising an amount of at least one chemical NOS inhibitor, wherein the at least one chemical NOS inhibitor comprises OKN-007 and/or OKN-007 sodium.

A composition comprising an amount of at least one chemical NOS inhibitor, wherein the at least one chemical NOS inhibitor comprises BYK-191023 and/or BYK-191023 dihydrochloride.

A composition comprising an amount of at least one chemical NOS inhibitor, wherein the at least one chemical NOS inhibitor comprises CM544 and/or CM544 dihydrochloride.

A composition comprising an amount of at least one chemical NOS inhibitor, wherein the at least one chemical NOS inhibitor is selected from the group consisting of 7-nitroindazole (“7-NI”), a salt of 7-NI, 3-bromo-7-nitroindazole (“3-Br-7-NI”), a salt of 3-Br-7-NI, N^{ω}-propyl-L-arginine (“L-NPA”), L-NPA hydrochloride, N⁵-(1-iminoethyl)-L-ornithine (“L-NIO”), L-NIO dihydrochloride, N-[4-[2-[[(3-chlorophenyl)methyl]amino]ethyl]phenyl]-2-thiophenecarboximidamide (“ARL-17477”), ARL-17477 dihydrochloride, N^{ω}-nitro-L-arginine (“L-NNA”), N^{ω}-nitro-L-arginine methyl ester (“L-NAME”), L-NAME hydrochloride, N^{ω}-methyl-L-arginine (“L-NMMA”), L-NMMA acetate, S-[2-[(1-iminoethyl)amino]ethyl]-L-homocysteine (“GW-274150”), GW-274150 phosphate, N⁶-(1-iminoethyl)-L-lysine (“L-NIL”), L-NIL hydrochloride, N-[[3-(aminomethyl)phenyl]methyl]-ethanimidamide (“1400W”), 1400W dihydrochloride, 2,4-disulfophenyl-N-tert-butyl nitrone (“OKN-007”), OKN-007 sodium, 2-[2-(4-methoxypyridin-2-yl)ethyl]-1H-imidazo[4,5-b]pyridine (“BYK-191023”), BYK-191023 dihydrochloride, N-{3-[(ethanimidoylamino)methyl]benzyl}-L-prolinamide (“CM544”), CM544 dihydrochloride, and any combination thereof, and wherein the composition is formulated to be administered to a subject via a route selected from the group consisting of buccally, inhalation, intraarterially, intraarticularly, intracardially, intracerebroventricularly, intradermally, intramedullary, intramuscularly, intraperitoneally, intrathecally, intravenously, intraventricularly, nasally, ophthalmically, orally, rectally, sublabially, sublingually, subcutaneously, topically, and transdermally.

A composition comprising an amount of at least one chemical NOS inhibitor, wherein the at least one chemical NOS inhibitor is selected from the group consisting of 7-nitroindazole (“7-NI”), a salt of 7-NI, 3-bromo-7-nitroindazole (“3-Br-7-NI”), a salt of 3-Br-7-NI, N^{ω}-propyl-L-arginine (“L-NPA”), L-NPA hydrochloride, N⁵-(1-iminoethyl)-L-ornithine (“L-NIO”), L-NIO dihydrochloride, N-[4-[2-[[(3-chlorophenyl)methyl]amino]ethyl]phenyl]-2-thiophenecarboximidamide (“ARL-17477”), ARL-17477 dihydrochloride, N^{ω}-nitro-L-arginine (“L-NNA”), N^{ω}-nitro-L-arginine methyl ester (“L-NAME”), L-NAME hydrochloride, N^{ω}-methyl-L-arginine (“L-NMMA”), L-NMMA acetate, S-[2-[(1-iminoethyl)amino]ethyl]-L-homocysteine (“GW-274150”), GW-274150 phosphate, N⁶-(1-iminoethyl)-L-lysine (“L-NIL”), L-NIL hydrochloride, N-[[3-(aminomethyl)phenyl]methyl]-ethanimidamide (“1400W”), 1400W dihydrochloride, 2,4-disulfophenyl-N-tert-butyl nitrone (“OKN-007”), OKN-007 sodium, 2-[2-(4-methoxypyridin-2-yl)ethyl]-1H-imidazo[4,5-b]pyridine (“BYK-191023”), BYK-191023 dihydrochloride, N-{3-[(ethanimidoylamino)methyl]benzyl}-L-prolinamide (“CM544”), CM544 dihydrochloride, and any combination thereof, and wherein the composition is formulated to be administered to a subject intraperitoneally.

A composition comprising an amount of at least one chemical NOS inhibitor, wherein the at least one chemical NOS inhibitor is selected from the group consisting of 7-nitroindazole (“7-NI”), a salt of 7-NI, 3-bromo-7-nitroindazole (“3-Br-7-NI”), a salt of 3-Br-7-NI, N^{ω}-propyl-L-arginine (“L-NPA”), L-NPA hydrochloride, N⁵-(1-iminoethyl)-L-ornithine (“L-NIO”), L-NIO dihydrochloride, N-[4-[2-[[(3-chlorophenyl)methyl]amino]ethyl]phenyl]-2-thiophenecarboximidamide (“ARL-17477”), ARL-17477 dihydrochloride, N^{ω}-nitro-L-arginine (“L-NNA”), N^{ω}-nitro-L-arginine methyl ester (“L-NAME”), L-NAME hydrochloride, N^{ω}-methyl-L-arginine (“L-NMMA”), L-NMMA acetate, S-[2-[(1-iminoethyl)amino]ethyl]-L-homocysteine (“GW-274150”), GW-274150 phosphate, N⁶-(1-iminoethyl)-L-lysine (“L-NIL”), L-NIL hydrochloride, N-[[3-(aminomethyl)phenyl]methyl]-ethanimidamide (“1400W”), 1400W dihydrochloride, 2,4-disulfophenyl-N-tert-butyl nitrone (“OKN-007”), OKN-007 sodium, 2-[2-(4-methoxypyridin-2-yl)ethyl]-1H-imidazo[4,5-b]pyridine (“BYK-191023”), BYK-191023 dihydrochloride, N-{3-[(ethanimidoylamino)methyl]benzyl}-L-prolinamide (“CM544”), CM544 dihydrochloride, and any combination thereof, and wherein the composition is formulated to be administered to a subject subcutaneously.

A composition comprising an amount of at least one chemical NOS inhibitor, wherein the at least one chemical NOS inhibitor is selected from the group consisting of 7-nitroindazole (“7-NI”), a salt of 7-NI, 3-bromo-7-nitroindazole (“3-Br-7-NI”), a salt of 3-Br-7-NI, N^{ω}-propyl-L-arginine (“L-NPA”), L-NPA hydrochloride, N⁵-(1-iminoethyl)-L-ornithine (“L-NIO”), L-NIO dihydrochloride, N-[4-[2-[[(3-chlorophenyl)methyl]amino]ethyl]phenyl]-2-thiophenecarboximidamide (“ARL-17477”), ARL-17477 dihydrochloride, N^{ω}-nitro-L-arginine (“L-NNA”), N^{ω}-nitro-L-arginine methyl ester (“L-NAME”), L-NAME hydrochloride, N^{ω}-methyl-L-arginine (“L-NMMA”), L-NMMA acetate, S-[2-[(1-iminoethyl)amino]ethyl]-L-homocysteine (“GW-274150”), GW-274150 phosphate, N⁶-(1-iminoethyl)-L-lysine (“L-NIL”), L-NIL hydrochloride, N-[[3-(aminomethyl)phenyl]methyl]-ethanimidamide (“1400W”), 1400W dihydrochloride, 2,4-disulfophenyl-N-tert-butyl nitrone (“OKN-007”), OKN-007 sodium, 2-[2-(4-methoxypyridin-2-yl)ethyl]-1H-imidazo[4,5-b]pyridine (“BYK-191023”), BYK-191023 dihydrochloride, N-{3-[(ethanimidoylamino)methyl]benzyl}-L-prolinamide (“CM544”), CM544 dihydrochloride, and any combination thereof, and wherein the composition is formulated to be administered to a subject intravenously.

A composition comprising an amount of at least one chemical NOS inhibitor, wherein the at least one chemical NOS inhibitor is selected from the group consisting of 7-nitroindazole (“7-NI”), a salt of 7-NI, 3-bromo-7-nitroindazole (“3-Br-7-NI”), a salt of 3-Br-7-NI, N^{ω}-propyl-L-arginine (“L-NPA”), L-NPA hydrochloride, N⁵-(1-iminoethyl)-L-ornithine (“L-NIO”), L-NIO dihydrochloride, N-[4-[2-[[(3-chlorophenyl)methyl]amino]ethyl]phenyl]-2-thiophenecarboximidamide (“ARL-17477”), ARL-17477 dihydrochloride, N^{ω}-nitro-L-arginine (“L-NNA”), N^{ω}-nitro-L-arginine methyl ester (“L-NAME”), L-NAME hydrochloride, N^{ω}-methyl-L-arginine (“L-NMMA”), L-NMMA acetate, S-[2-[(1-iminoethyl)amino]ethyl]-L-homocysteine (“GW-274150”), GW-274150 phosphate, N⁶-(1-iminoethyl)-L-lysine (“L-NIL”), L-NIL hydrochloride, N-[[3-(aminomethyl)phenyl]methyl]-ethanimidamide (“1400W”), 1400W dihydrochloride, 2,4-disulfophenyl-N-tert-butyl nitrone (“OKN-007”), OKN-007 sodium, 2-[2-(4-methoxypyridin-2-yl)ethyl]-1H-imidazo[4,5-b]pyridine (“BYK-191023”), BYK-191023 dihydrochloride, N-{3-[(ethanimidoylamino)methyl]benzyl}-L-prolinamide (“CM544”), CM544 dihydrochloride, and any combination thereof, wherein the composition is formulated to be administered to a subject via a route selected from the group consisting of buccally, inhalation, intraarterially, intraarticularly, intracardially, intracerebroventricularly, intradermally, intramedullary, intramuscularly, intraperitoneally, intrathecally, intravenously, intraventricularly, nasally, ophthalmically, orally, rectally, sublabially, sublingually, subcutaneously, topically, and transdermally, and wherein the amount of the at least one chemical NOS inhibitor, when administered to the subject, provides a dosage of the at least one chemical NOS inhibitor of about 80 μg/kg to about 10 mg/kg to the subject.

A composition comprising an amount of at least one chemical NOS inhibitor, wherein the at least one chemical NOS inhibitor is selected from the group consisting of 7-nitroindazole (“7-NI”), a salt of 7-NI, 3-bromo-7-nitroindazole (“3-Br-7-NI”), a salt of 3-Br-7-NI, N^{ω}-propyl-L-arginine (“L-NPA”), L-NPA hydrochloride, N⁵-(1-iminoethyl)-L-ornithine (“L-NIO”), L-NIO dihydrochloride, N-[4-[2-[[(3-chlorophenyl)methyl]amino]ethyl]phenyl]-2-thiophenecarboximidamide (“ARL-17477”), ARL-17477 dihydrochloride, N^{ω}-nitro-L-arginine (“L-NNA”), N^{ω}-nitro-L-arginine methyl ester (“L-NAME”), L-NAME hydrochloride, N^{ω}-methyl-L-arginine (“L-NMMA”), L-NMMA acetate, S-[2-1[(1-iminoethyl)amino]ethyl]-L-homocysteine (“GW-274150”), GW-274150 phosphate, N⁶-(1-iminoethyl)-L-lysine (“L-NIL”), L-NIL hydrochloride, N-[[3-(aminomethyl)phenyl]methyl]-ethanimidamide (“1400W”), 1400W dihydrochloride, 2,4-disulfophenyl-N-tert-butyl nitrone (“OKN-007”), OKN-007 sodium, 2-[2-(4-methoxypyridin-2-yl)ethyl]-1H-imidazo[4,5-b]pyridine (“BYK-191023”), BYK-191023 dihydrochloride, N-{3-[(ethanimidoylamino)methyl]benzyl}-L-prolinamide (“CM544”), CM544 dihydrochloride, and any combination thereof, wherein the composition is formulated to be administered to a subject via a route selected from the group consisting of buccally, inhalation, intraarterially, intraarticularly, intracardially, intracerebroventricularly, intradermally, intramedullary, intramuscularly, intraperitoneally, intrathecally, intravenously, intraventricularly, nasally, ophthalmically, orally, rectally, sublabially, sublingually, subcutaneously, topically, and transdermally, and wherein the amount of the at least one chemical NOS inhibitor, when administered to the subject, provides a dosage of the at least one chemical NOS inhibitor of about 800 μg/kg to the subject.

A composition comprising an amount of at least one chemical NOS inhibitor, wherein the at least one chemical NOS inhibitor is selected from the group consisting of 7-nitroindazole (“7-NI”), a salt of 7-NI, 3-bromo-7-nitroindazole (“3-Br-7-NI”), a salt of 3-Br-7-NI, N^{ω}-propyl-L-arginine (“L-NPA”), L-NPA hydrochloride, N⁵-(1-iminoethyl)-L-ornithine (“L-NIO”), L-NIO dihydrochloride, N-[4-[2-[[(3-chlorophenyl)methyl]amino]ethyl]phenyl]-2-thiophenecarboximidamide (“ARL-17477”), ARL-17477 dihydrochloride, N^{ω}-nitro-L-arginine (“L-NNA”), N^{ω}-nitro-L-arginine methyl ester (“L-NAME”), L-NAME hydrochloride, N^{ω}-methyl-L-arginine (“L-NMMA”), L-NMMA acetate, S-[2-[(1-iminoethyl)amino]ethyl]-L-homocysteine (“GW-274150”), GW-274150 phosphate, N⁶-(1-iminoethyl)-L-lysine (“L-NIL”), L-NIL hydrochloride, N-[[3-(aminomethyl)phenyl]methyl]-ethanimidamide (“1400W”), 1400W dihydrochloride, 2,4-disulfophenyl-N-tert-butyl nitrone (“OKN-007”), OKN-007 sodium, 2-[2-(4-methoxypyridin-2-yl)ethyl]-1H-imidazo[4,5-b]pyridine (“BYK-191023”), BYK-191023 dihydrochloride, N-{3-[(ethanimidoylamino)methyl]benzyl}-L-prolinamide (“CM544”), CM544 dihydrochloride, and any combination thereof, wherein the composition is formulated to be administered to a subject via a route selected from the group consisting of buccally, inhalation, intraarterially, intraarticularly, intracardially, intracerebroventricularly, intradermally, intramedullary, intramuscularly, intraperitoneally, intrathecally, intravenously, intraventricularly, nasally, ophthalmically, orally, rectally, sublabially, sublingually, subcutaneously, topically, and transdermally, and wherein the amount of the at least one chemical NOS inhibitor, when administered to the subject, provides a dosage of the at least one chemical NOS inhibitor of about 3 mg/kg to the subject.

A composition comprising an amount of at least one chemical NOS inhibitor, wherein the at least one chemical NOS inhibitor is selected from the group consisting of 7-nitroindazole (“7-NI”), a salt of 7-NI, 3-bromo-7-nitroindazole (“3-Br-7-NI”), a salt of 3-Br-7-NI, N^{ω}-propyl-L-arginine (“L-NPA”), L-NPA hydrochloride, N⁵-(1-iminoethyl)-L-ornithine (“L-NIO”), L-NIO dihydrochloride, N-[4-[2-[[(3-chlorophenyl)methyl]amino]ethyl]phenyl]-2-thiophenecarboximidamide (“ARL-17477”), ARL-17477 dihydrochloride, N^{ω}-nitro-L-arginine (“L-NNA”), N^{ω}-nitro-L-arginine methyl ester (“L-NAME”), L-NAME hydrochloride, N^{ω}-methyl-L-arginine (“L-NMMA”), L-NMMA acetate, S-[2-1[(1-iminoethyl)amino]ethyl]-L-homocysteine (“GW-274150”), GW-274150 phosphate, N⁶-(1-iminoethyl)-L-lysine (“L-NIL”), L-NIL hydrochloride, N-[[3-(aminomethyl)phenyl]methyl]-ethanimidamide (“1400W”), 1400W dihydrochloride, 2,4-disulfophenyl-N-tert-butyl nitrone (“OKN-007”), OKN-007 sodium, 2-[2-(4-methoxypyridin-2-yl)ethyl]-1H-imidazo[4,5-b]pyridine (“BYK-191023”), BYK-191023 dihydrochloride, N-{3-[(ethanimidoylamino)methyl]benzyl}-L-prolinamide (“CM544”), CM544 dihydrochloride, and any combination thereof, wherein the composition is formulated to be administered to a subject via a route selected from the group consisting of buccally, inhalation, intraarterially, intraarticularly, intracardially, intracerebroventricularly, intradermally, intramedullary, intramuscularly, intraperitoneally, intrathecally, intravenously, intraventricularly, nasally, ophthalmically, orally, rectally, sublabially, sublingually, subcutaneously, topically, and transdermally, and wherein the amount of the at least one chemical NOS inhibitor, when administered to the subject, provides a dosage of the at least one chemical NOS inhibitor of about 6 mg/kg to the subject.

A method of treating, ameliorating, and/or reducing the severity of a cancer in a subject, the method comprising: identifying that the subject has the cancer, or a symptom associated therewith; administering a composition to the subject, wherein the composition comprises an amount of at least one chemical NOS inhibitor.

A method of treating, ameliorating, and/or reducing the severity of a cancer in a subject, the method comprising: identifying that the subject has the cancer, or a symptom associated therewith; administering a composition to the subject, wherein the composition comprises an amount of at least one chemical NOS inhibitor, and wherein the at least one chemical NOS inhibitor is selected from the group consisting of 7-nitroindazole (“7-NI”), a salt of 7-NI, 3-bromo-7-nitroindazole (“3-Br-7-NI”), a salt of 3-Br-7-NI, N^{ω}-propyl-L-arginine (“L-NPA”), L-NPA hydrochloride, N⁵-(1-iminoethyl)-L-ornithine (“L-NIO”), L-NIO dihydrochloride, N-[4-[2-[[(3-chlorophenyl)methyl]amino]ethyl]phenyl]-2-thiophenecarboximidamide (“ARL-17477”), ARL-17477 dihydrochloride, N^{ω}-nitro-L-arginine (“L-NNA”), N^{ω}-nitro-L-arginine methyl ester (“L-NAME”), L-NAME hydrochloride, N^{ω}-methyl-L-arginine (“L-NMMA”), L-NMMA acetate, S-[2-[(1-iminoethyl)amino]ethyl]-L-homocysteine (“GW-274150”), GW-274150 phosphate, N⁶-(1-iminoethyl)-L-lysine (“L-NIL”), L-NIL hydrochloride, N-[[3-(aminomethyl)phenyl]methyl]-ethanimidamide (“1400W”), 1400W dihydrochloride, 2,4-disulfophenyl-N-tert-butyl nitrone (“OKN-007”), OKN-007 sodium, 2-[2-(4-methoxypyridin-2-yl)ethyl]-1H-imidazo[4,5-b]pyridine (“BYK-191023”), BYK-191023 dihydrochloride, N-{3-[(ethanimidoylamino)methyl]benzyl}-L-prolinamide (“CM544”), CM544 dihydrochloride, and any combination thereof.

A method of treating, ameliorating, and/or reducing the severity of a cancer in a subject, the method comprising: identifying that the subject has the cancer, or a symptom associated therewith; administering a composition to the subject, wherein the composition comprises an amount of at least one chemical NOS inhibitor, and wherein the at least one chemical NOS inhibitor comprises 7-NI, a salt of 7-NI, and/or a metal complex of 7-NI.

A method of treating, ameliorating, and/or reducing the severity of a cancer in a subject, the method comprising: identifying that the subject has the cancer, or a symptom associated therewith; administering a composition to the subject, wherein the composition comprises an amount of at least one chemical NOS inhibitor, and wherein the at least one chemical NOS inhibitor comprises 3-Br-7-NI and/or a salt of 3-Br-7-NI.

A method of treating, ameliorating, and/or reducing the severity of a cancer in a subject, the method comprising: identifying that the subject has the cancer, or a symptom associated therewith; administering a composition to the subject, wherein the composition comprises an amount of at least one chemical NOS inhibitor, and wherein the at least one chemical NOS inhibitor comprises L-NPA and/or L-NPA hydrochloride.

A method of treating, ameliorating, and/or reducing the severity of a cancer in a subject, the method comprising: identifying that the subject has the cancer, or a symptom associated therewith; administering a composition to the subject, wherein the composition comprises an amount of at least one chemical NOS inhibitor, and wherein the at least one chemical NOS inhibitor comprises L-NIO and/or L-NIO dihydrochloride.

A method of treating, ameliorating, and/or reducing the severity of a cancer in a subject, the method comprising: identifying that the subject has the cancer, or a symptom associated therewith; administering a composition to the subject, wherein the composition comprises an amount of at least one chemical NOS inhibitor, and wherein the at least one chemical NOS inhibitor comprises ARL-17477 and/or ARL-17477 dihydrochloride.

A method of treating, ameliorating, and/or reducing the severity of a cancer in a subject, the method comprising: identifying that the subject has the cancer, or a symptom associated therewith; administering a composition to the subject, wherein the composition comprises an amount of at least one chemical NOS inhibitor, and wherein the at least one chemical NOS inhibitor comprises L-NNA and/or a salt of L-NNA.

A method of treating, ameliorating, and/or reducing the severity of a cancer in a subject, the method comprising: identifying that the subject has the cancer, or a symptom associated therewith; administering a composition to the subject, wherein the composition comprises an amount of at least one chemical NOS inhibitor, and wherein the at least one chemical NOS inhibitor comprises L-NAME and/or L-NAME hydrochloride.

A method of treating, ameliorating, and/or reducing the severity of a cancer in a subject, the method comprising: identifying that the subject has the cancer, or a symptom associated therewith; administering a composition to the subject, wherein the composition comprises an amount of at least one chemical NOS inhibitor, and wherein the at least one chemical NOS inhibitor comprises L-NMMA and/or L-NMMA acetate.

A method of treating, ameliorating, and/or reducing the severity of a cancer in a subject, the method comprising: identifying that the subject has the cancer, or a symptom associated therewith; administering a composition to the subject, wherein the composition comprises an amount of at least one chemical NOS inhibitor, and wherein the at least one chemical NOS inhibitor comprises GW-274150 and/or GW-274150 phosphate.

A method of treating, ameliorating, and/or reducing the severity of a cancer in a subject, the method comprising: identifying that the subject has the cancer, or a symptom associated therewith; administering a composition to the subject, wherein the composition comprises an amount of at least one chemical NOS inhibitor, and wherein the at least one chemical NOS inhibitor comprises L-NIL and/or L-NIL hydrochloride.

A method of treating, ameliorating, and/or reducing the severity of a cancer in a subject, the method comprising: identifying that the subject has the cancer, or a symptom associated therewith; administering a composition to the subject, wherein the composition comprises an amount of at least one chemical NOS inhibitor, and wherein the at least one chemical NOS inhibitor comprises L-NAME and/or L-NAME hydrochloride.

A method of treating, ameliorating, and/or reducing the severity of a cancer in a subject, the method comprising: identifying that the subject has the cancer, or a symptom associated therewith; administering a composition to the subject, wherein the composition comprises an amount of at least one chemical NOS inhibitor, and wherein the at least one chemical NOS inhibitor comprises 1400W and/or 1400W dihydrochloride.

A method of treating, ameliorating, and/or reducing the severity of a cancer in a subject, the method comprising: identifying that the subject has the cancer, or a symptom associated therewith; administering a composition to the subject, wherein the composition comprises an amount of at least one chemical NOS inhibitor, and wherein the at least one chemical NOS inhibitor comprises OKN-007 and/or OKN-007 sodium.

A method of treating, ameliorating, and/or reducing the severity of a cancer in a subject, the method comprising: identifying that the subject has the cancer, or a symptom associated therewith; administering a composition to the subject, wherein the composition comprises an amount of at least one chemical NOS inhibitor, and wherein the at least one chemical NOS inhibitor comprises BYK-191023 and/or BYK-191023 dihydrochloride.

A method of treating, ameliorating, and/or reducing the severity of a cancer in a subject, the method comprising: identifying that the subject has the cancer, or a symptom associated therewith; administering a composition to the subject, wherein the composition comprises an amount of at least one chemical NOS inhibitor, and wherein the at least one chemical NOS inhibitor comprises CM544 and/or CM544 dihydrochloride.

A method of treating, ameliorating, and/or reducing the severity of a cancer in a subject, the method comprising: identifying that the subject has the cancer, or a symptom associated therewith; administering a composition to the subject, wherein the composition comprises an amount of at least one chemical NOS inhibitor, and wherein the composition is administered to the subject via a route selected from the group consisting of buccally, inhalation, intraarterially, intraarticularly, intracardially, intracerebroventricularly, intradermally, intramedullary, intramuscularly, intraperitoneally, intrathecally, intravenously, intraventricularly, nasally, ophthalmically, orally, rectally, sublabially, sublingually, subcutaneously, topically, and transdermally.

A method of treating, ameliorating, and/or reducing the severity of a cancer in a subject, the method comprising: identifying that the subject has the cancer, or a symptom associated therewith; administering a composition to the subject, wherein the composition comprises an amount of at least one chemical NOS inhibitor, and wherein the composition is administered to the subject intraperitoneally.

A method of treating, ameliorating, and/or reducing the severity of a cancer in a subject, the method comprising: identifying that the subject has the cancer, or a symptom associated therewith; administering a composition to the subject, wherein the composition comprises an amount of at least one chemical NOS inhibitor, and wherein the composition is administered to the subject subcutaneously.

A method of treating, ameliorating, and/or reducing the severity of a cancer in a subject, the method comprising: identifying that the subject has the cancer, or a symptom associated therewith; administering a composition to the subject, wherein the composition comprises an amount of at least one chemical NOS inhibitor, and wherein the composition is administered to the subject intravenously.

A method of treating, ameliorating, and/or reducing the severity of a cancer in a subject, the method comprising: identifying that the subject has the cancer, or a symptom associated therewith; administering a composition to the subject, wherein the composition comprises an amount of at least one chemical NOS inhibitor, and wherein the composition is administered to the subject daily.

A method of treating, ameliorating, and/or reducing the severity of a cancer in a subject, the method comprising: identifying that the subject has the cancer, or a symptom associated therewith; administering a composition to the subject, wherein the composition comprises an amount of at least one chemical NOS inhibitor, and wherein the composition is administered to the subject every three days.

A method of treating, ameliorating, and/or reducing the severity of a cancer in a subject, the method comprising: identifying that the subject has the cancer, or a symptom associated therewith; administering a composition to the subject, wherein the composition comprises an amount of at least one chemical NOS inhibitor, and wherein the amount of the at least one chemical NOS inhibitor, when administered to the subject, provides a dosage of the at least one chemical NOS inhibitor of about 80 μg/kg to about 10 mg/kg to the subject.

A method of treating, ameliorating, and/or reducing the severity of a cancer in a subject, the method comprising: identifying that the subject has the cancer, or a symptom associated therewith; administering a composition to the subject, wherein the composition comprises an amount of at least one chemical NOS inhibitor, and wherein the amount of the at least one chemical NOS inhibitor, when administered to the subject, provides a dosage of the at least one chemical NOS inhibitor of about 800 μg/kg to the subject.

A method of treating, ameliorating, and/or reducing the severity of a cancer in a subject, the method comprising: identifying that the subject has the cancer, or a symptom associated therewith; administering a composition to the subject, wherein the composition comprises an amount of at least one chemical NOS inhibitor, and wherein the amount of the at least one chemical NOS inhibitor, when administered to the subject, provides a dosage of the at least one chemical NOS inhibitor of about 3 mg/kg to the subject.

A method of treating, ameliorating, and/or reducing the severity of a cancer in a subject, the method comprising: identifying that the subject has the cancer, or a symptom associated therewith; administering a composition to the subject, wherein the composition comprises an amount of at least one chemical NOS inhibitor, and wherein the amount of the at least one chemical NOS inhibitor, when administered to the subject, provides a dosage of the at least one chemical NOS inhibitor of about 6 mg/kg to the subject.

A method of treating, ameliorating, and/or reducing the severity of a cancer in a subject, the method comprising: identifying that the subject has the cancer, or a symptom associated therewith; administering a composition to the subject, wherein the composition comprises an amount of at least one chemical NOS inhibitor, and wherein subject is a child.

A method of treating, ameliorating, and/or reducing the severity of a cancer in a subject, the method comprising: identifying that the subject has the cancer, or a symptom associated therewith; administering a composition to the subject, wherein the composition comprises an amount of at least one chemical NOS inhibitor, and wherein the subject is an adult.

A method of treating, ameliorating, and/or reducing the severity of a cancer in a subject, the method comprising: identifying that the subject has the cancer, or a symptom associated therewith; administering a composition to the subject, wherein the composition comprises an amount of at least one chemical NOS inhibitor, and wherein the cancer is glioblastoma.

A method of treating, ameliorating, and/or reducing the severity of a cancer in a subject, the method comprising: identifying that the subject has the cancer, or a symptom associated therewith; administering a composition to the subject, wherein the composition comprises an amount of at least one chemical NOS inhibitor, and wherein the cancer is neuroblastoma.

A method of treating, ameliorating, and/or reducing the severity of a cancer in a subject, the method comprising: identifying that the subject has the cancer, or a symptom associated therewith; administering a composition to the subject, wherein the composition comprises an amount of at least one chemical NOS inhibitor, and wherein the composition is administered after a surgery to remove or resect a portion of the cancer has been performed on the subject.

A method of treating, ameliorating, and/or reducing the severity of a cancer in a subject, the method comprising: identifying that the subject has the cancer, or a symptom associated therewith; administering a composition to the subject, wherein the composition comprises an amount of at least one chemical NOS inhibitor, and wherein the composition is administered prior to a surgery to remove or resect a portion of the cancer being performed on the subject.

A method of treating, ameliorating, and/or reducing the severity of a cancer in a subject, the method comprising: identifying that the subject has the cancer, or a symptom associated therewith; administering a composition to the subject, wherein the composition comprises an amount of at least one chemical NOS inhibitor, and wherein the composition comprises at least two chemical NOS inhibitors.

A method of treating, ameliorating, and/or reducing the severity of a cancer in a subject, the method comprising: identifying that the subject has the cancer, or a symptom associated therewith; administering a composition to the subject, wherein the composition comprises an amount of at least one chemical NOS inhibitor, wherein the composition comprises at least two chemical NOS inhibitors, and wherein the at least two chemical NOS inhibitors are administered to the subject concurrently.

A method of treating, ameliorating, and/or reducing the severity of a cancer in a subject, the method comprising: identifying that the subject has the cancer, or a symptom associated therewith; administering a composition to the subject, wherein the composition comprises an amount of at least one chemical NOS inhibitor, wherein the composition comprises at least two chemical NOS inhibitors, and wherein the at least two chemical NOS inhibitors are administered to the subject at different times.

A method of treating, ameliorating, and/or reducing the severity of a cancer in a subject, the method comprising: identifying that the subject has the cancer, or a symptom associated therewith; administering a composition to the subject, wherein the composition comprises an amount of at least one chemical NOS inhibitor, wherein the composition comprises an amount of at least one chemical NOS inhibitor, wherein the composition comprises at least two chemical NOS inhibitors, and wherein the at least two chemical NOS inhibitors are administered to the subject via a same route of administration.

A method of treating, ameliorating, and/or reducing the severity of a cancer in a subject, the method comprising: identifying that the subject has the cancer, or a symptom associated therewith; administering a composition to the subject, wherein the composition comprises an amount of at least one chemical NOS inhibitor, wherein the composition comprises an amount of at least one chemical NOS inhibitor, wherein the composition comprises at least two chemical NOS inhibitors, and wherein the at least two chemical NOS inhibitors are administered to the subject via different routes of administration.

A method of treating, ameliorating, and/or reducing the severity of a cancer in a subject, the method comprising: identifying that the subject has the cancer, or a symptom associated therewith; administering a composition to the subject, wherein the composition comprises an amount of at least one chemical NOS inhibitor, and wherein the composition is administered with at least one chemotherapy selected from the group consisting of abemaciclib, abiraterone acetate, acalabrutinib, adagrasib, afatinib dimaleate, albumin-bound paclitaxel, albumin-bound rapamycin (Sirolimus Protein-Bound Particles), alectinib, alpelisib, amifostine, aminolevulinic acid hydrochloride, anastrozole, apalutamide, aprepitant, arsenic trioxide, asciminib hydrochloride, avapritinib, axitinib, azacytidine, belinostat, belzutifan, bendamustine hydrochloride, bexarotene, bicalutamide, binimetinib, bleomycin, bleomycin sulfate, bortezomib, bosutinib, brigatinib, busulfan, cabazitaxel, cabozantinib-S-malate, capecitabine, capmatinib hydrochloride, carboplatin, carfilzomib, carmustine, cedazuridine, ceritinib, chlorambucil, cisplatin, cladribine, clofarabine, cobimetinib fumarate, copanlisib hydrochloride, crizotinib, cyclophosphamide, cytarabine, dabrafenib mesylate, dacarbazine, dacomitinib, dactinomycin, darolutamide, dasatinib, daunorubicin hydrochloride, decitabine, defibrotide sodium, degarelix, dexamethasone, dexrazoxane hydrochloride, docetaxel, doxorubicin hydrochloride, duvelisib, elacestrant dihydrochloride, eltrombopag olamine, enasidenib mesylate, encorafenib, entrectinib, enzalutamide, epirubicin hydrochloride, erdafitinib, eribulin mesylate, erlotinib hydrochloride, etoposide, etoposide phosphate, everolimus, exemestane, fedratinib hydrochloride, fludarabine phosphate, fluorouracil, flutamide, folinic acid, fostamatinib disodium, fulvestrant, futibatinib, gefitinib, gemcitabine hydrochloride, gilteritinib fumarate, glasdegib maleate, goserelin acetate, granisetron, granisetron hydrochloride, hydroxyurea, ibrutinib, idarubicin hydrochloride, idelalisib, ifosfamide, imatinib mesylate, imiquimod, infigratinib phosphate, iobenguane I-131, irinotecan hydrochloride, ivosidenib, ixabepilone, ixazomib citrate, lanreotide acetate, lapatinib ditosylate, larotrectinib sulfate, lenalidomide, lenvatinib mesylate, letrozole, leucovorin calcium, leuprolide acetate, liposomal cytarabine, liposomal doxorubicin hydrochloride, liposomal irinotecan hydrochloride, lomustine, lorlatinib, lurbinectedin, Lu-177 dotatate, Lu-177 vipivotide tetraxetan, mechlorethamine hydrochloride, megestrol acetate, melphalan, melphalan hydrochloride, mercaptopurine, mesna, methotrexate, methotrexate sodium, methylnaltrexone bromide, midostaurin, mitomycin, mitoxantrone hydrochloride, mobocertinib succinate, nelarabine, neratinib maleate, netupitant hydrochloride, nilutamide, nilotinib, niraparib tosylate monohydrate, olaparib, olutasidenib, omacetaxine mepesuccinate, ondansetron hydrochloride, osimertinib mesylate, oxaliplatin, paclitaxel, pacritinib citrate, palbociclib, palonosetron hydrochloride, pamidronate disodium, pazopanib hydrochloride, pemetrexed disodium, pemigatinib, pexidartinib hydrochloride, pirtobrutinib, plerixafor, pomalidomide, ponatinib hydrochloride, pralatrexate, pralsetinib, prednisone, procarbazine hydrochloride, propranolol hydrochloride, Ra-223 dichloride, raloxifene hydrochloride, regorafenib, relugolix, ribociclib, ripretinib, rolapitant hydrochloride, romidepsin, rucaparib camsylate, ruxolitinib phosphate, selinexor, selpercatinib, selumetinib sulfate, sonidegib, sorafenib tosylate, sotorasib, sunitinib malate, talazoparib tosylate, talc, tamoxifen citrate, tazemetostat hydrobromide, temozolomide, temsirolimus, tepotinib hydrochloride, thalidomide, thioguanine, thiotepa, tipiracil hydrochloride, tivozanib hydrochloride, topotecan hydrochloride, toremifene, trabectedin, trametinib dimethyl sulfoxide, tretinoin, trifluridine hydrochloride, tucatinib, uridine triacetate, valrubicin, vandetanib, vemurafenib, venetoclax, vinblastine sulfate, vincristine sulfate, vinorelbine tartrate, vismodegib, vorinostat, zanubrutinib, zoledronic acid, and any combination thereof.

A method of treating, ameliorating, and/or reducing the severity of a cancer in a subject, the method comprising: identifying that the subject has the cancer, or a symptom associated therewith; administering a composition to the subject, wherein the composition comprises an amount of at least one chemical NOS inhibitor, and wherein the composition is administered with at least one immunotherapy, cell-based therapy, and/or gene-based therapy selected from the group consisting of ado-trastuzumab emtansine, aldesleukin, alemtuzumab, amivantamab-vmjw, asparaginase Erwinia chrysanthemi, asparaginase Erwinia chrysanthemi (recombinant)-rywn, atezolizumab, avelumab, axicabtagene ciloleucel, bevacizumab, blinatumomab, brentuximab vedotin, brexucabtagene autoleucel, calaspargase pegol-mknl, camrelizumab, caplacizumab-yhdp, cemiplimab-rwlc, cetuximab, ciltacabtagene autoleucel, daratumumab, daratumumab and hyaluronidase-fihj, darbepoetin alfa, denileukin diftitox, denosumab, dinutuximab, dostarlimab-gxly, durvalumab, elotuzumab, emapalumab-lzsg, enfortumab vedotin-ejfv, epoetin alfa, fam-trastuzumab deruxtecan-nxki, filgrastim, gemtuzumab ozogamicin, glucarpidase, ibritumomab tiuxetan, idecabtagene vicleucel, inotuzumab ozogamicin, ipilimumab, isatuximab-irfc, lisocabtagene maraleucel, loncastuximab tesirine-lpyl, luspatercept-aamt, margetuximab-cmkb, mirvetuximab soravtansine-gynx, mogamulizumab-kpkc, mosunetuzumab-axgb, moxetumomab pasudotox-tdfk, nadofaragene firadenovec-vncg, naxitamab-gqgk, necitumumab, nivolumab, nivolumab and relatlimab-rmbw, obinutuzumab, ofatumumab, palifermin, panitumumab, pegaspargase, pegfilgrastim, pembrolizumab, pertuzumab, pertuzumab, trastuzumab, and hyaluronidase-zzxf, polatuzumab vedotin-piiq, ramucirumab, rasburicase, ravulizumab-cwvz, recombinant HPV bivalent vaccine, recombinant HPV nonavalent vaccine, recombinant HPV quadrivalent vaccine, recombinant interferon alfa-2b, rituximab, rituximab and hyaluronidase human, romiplostim, ropeginterferon alfa-2b-njft, sacituzumab govitecan-hziy, siltuximab, sintilimab, sipuleucel-T, tafasitamab-cxix, tagraxofusp-erzs, talimogene laherparepvec, tebentafusp-tebn, teclistamab-cqyv, tisagenlecleucel, tislelizumab, tisotumab vedotin-tftv, tocilizumab, toripalimab, trastuzumab, trastuzumab and hyaluronidase-oysk, tremelimumab-actl, ziv-aflibercept, and any combination thereof.

A composition for use in treating neuroblastoma, wherein the composition comprises an amount of at least one chemical NOS inhibitor, wherein the at least one chemical NOS inhibitor is selected from the group consisting of comprising an amount of at least one chemical NOS inhibitor, wherein the at least one chemical NOS inhibitor is selected from the group consisting of 7-nitroindazole (“7-NI”), a salt of 7-NI, 3-bromo-7-nitroindazole (“3-Br-7-NI”), a salt of 3-Br-7-NI, N^{ω}-propyl-L-arginine (“L-NPA”), L-NPA hydrochloride, N⁵-(1-iminoethyl)-L-ornithine (“L-NIO”), L-NIO dihydrochloride, N-[4-[2-[[(3-chlorophenyl)methyl]amino]ethyl]phenyl]-2-thiophenecarboximidamide (“ARL-17477”), ARL-17477 dihydrochloride, N^{ω}-nitro-L-arginine (“L-NNA”), N^{ω}-nitro-L-arginine methyl ester (“L-NAME”), L-NAME hydrochloride, N^{ω}-methyl-L-arginine (“L-NMMA”), L-NMMA acetate, S-[2-[(1-iminoethyl)amino]ethyl]-L-homocysteine (“GW-274150”), GW-274150 phosphate, N⁶-(1-iminoethyl)-L-lysine (“L-NIL”), L-NIL hydrochloride, N-[[3-(aminomethyl)phenyl]methyl]-ethanimidamide (“1400W”), 1400W dihydrochloride, 2,4-disulfophenyl-N-tert-butyl nitrone (“OKN-007”), OKN-007 sodium, 2-[2-(4-methoxypyridin-2-yl)ethyl]-1H-imidazo[4,5-b]pyridine (“BYK-191023”), BYK-191023 dihydrochloride, N-{3-[(ethanimidoylamino)methyl]benzyl}-L-prolinamide (“CM544”), CM544 dihydrochloride, and any combination thereof.

A composition for use in treating glioblastoma, wherein the composition comprises an amount of at least one chemical NOS inhibitor, wherein the at least one chemical NOS inhibitor is selected from the group consisting of 7-nitroindazole (“7-NI”), a salt of 7-NI, 3-bromo-7-nitroindazole (“3-Br-7-NI”), a salt of 3-Br-7-NI, N^{ω}-propyl-L-arginine (“L-NPA”), L-NPA hydrochloride, N⁵-(1-iminoethyl)-L-ornithine (“L-NIO”), L-NIO dihydrochloride, N-[4-[2-[[(3-chlorophenyl)methyl]amino]ethyl]phenyl]-2-thiophenecarboximidamide (“ARL-17477”), ARL-17477 dihydrochloride, N^{ω}-nitro-L-arginine (“L-NNA”), N^{ω}-nitro-L-arginine methyl ester (“L-NAME”), L-NAME hydrochloride, N^{ω}-methyl-L-arginine (“L-NMMA”), L-NMMA acetate, S-[2-[(1-iminoethyl)amino]ethyl]-L-homocysteine (“GW-274150”), GW-274150 phosphate, N⁶-(1-iminoethyl)-L-lysine (“L-NIL”), L-NIL hydrochloride, N-[[3-(aminomethyl)phenyl]methyl]-ethanimidamide (“1400W”), 1400W dihydrochloride, 2,4-disulfophenyl-N-tert-butyl nitrone (“OKN-007”), OKN-007 sodium, 2-[2-(4-methoxypyridin-2-yl)ethyl]-1H-imidazo[4,5-b]pyridine (“BYK-191023”), BYK-191023 dihydrochloride, N-{3-[(ethanimidoylamino)methyl]benzyl}-L-prolinamide (“CM544”), CM544 dihydrochloride, and any combination thereof.

A composition for use in treating pancreatic cancer, wherein the composition comprises an amount of at least one chemical NOS inhibitor, wherein the at least one chemical NOS inhibitor is selected from the group consisting of 7-nitroindazole (“7-NI”), a salt of 7-NI, 3-bromo-7-nitroindazole (“3-Br-7-NI”), a salt of 3-Br-7-NI, N^{ω}-propyl-L-arginine (“L-NPA”), L-NPA hydrochloride, N⁵-(1-iminoethyl)-L-ornithine (“L-NIO”), L-NIO dihydrochloride, N-[4-[2-[[(3-chlorophenyl)methyl]amino]ethyl]phenyl]-2-thiophenecarboximidamide (“ARL-17477”), ARL-17477 dihydrochloride, N^{ω}-nitro-L-arginine (“L-NNA”), N^{ω}-nitro-L-arginine methyl ester (“L-NAME”), L-NAME hydrochloride, N^{ω}-methyl-L-arginine (“L-NMMA”), L-NMMA acetate, S-[2-[(1-iminoethyl)amino]ethyl]-L-homocysteine (“GW-274150”), GW-274150 phosphate, N⁶-(1-iminoethyl)-L-lysine (“L-NIL”), L-NIL hydrochloride, N-[[3-(aminomethyl)phenyl]methyl]-ethanimidamide (“1400W”), 1400W dihydrochloride, 2,4-disulfophenyl-N-tert-butyl nitrone (“OKN-007”), OKN-007 sodium, 2-[2-(4-methoxypyridin-2-yl)ethyl]-1H-imidazo[4,5-b]pyridine (“BYK-191023”), BYK-191023 dihydrochloride, N-{3-[(ethanimidoylamino)methyl]benzyl}-L-prolinamide (“CM544”), CM544 dihydrochloride, and any combination thereof.

A composition for use in treating gastrointestinal cancer, wherein the composition comprises an amount of at least one chemical NOS inhibitor, wherein the at least one chemical NOS inhibitor is selected from the group consisting of 7-nitroindazole (“7-NI”), a salt of 7-NI, 3-bromo-7-nitroindazole (“3-Br-7-NI”), a salt of 3-Br-7-NI, N^{ω}-propyl-L-arginine (“L-NPA”), L-NPA hydrochloride, N⁵-(1-iminoethyl)-L-ornithine (“L-NIO”), L-NIO dihydrochloride, N-[4-[2-[1[(3-chlorophenyl)methyl]amino]ethyl]phenyl]-2-thiophenecarboximidamide (“ARL-17477”), ARL-17477 dihydrochloride, N^{ω}-nitro-L-arginine (“L-NNA”), N^{ω}-nitro-L-arginine methyl ester (“L-NAME”), L-NAME hydrochloride, N^{ω}-methyl-L-arginine (“L-NMMA”), L-NMMA acetate, S-[2-[(1-iminoethyl)amino]ethyl]-L-homocysteine (“GW-274150”), GW-274150 phosphate, N⁶-(1-iminoethyl)-L-lysine (“L-NIL”), L-NIL hydrochloride, N-[[3-(aminomethyl)phenyl]methyl]-ethanimidamide (“1400W”), 1400W dihydrochloride, 2,4-disulfophenyl-N-tert-butyl nitrone (“OKN-007”), OKN-007 sodium, 2-[2-(4-methoxypyridin-2-yl)ethyl]-1H-imidazo[4,5-b]pyridine (“BYK-191023”), BYK-191023 dihydrochloride, N-{3-[(ethanimidoylamino)methyl]benzyl}-L-prolinamide (“CM544”), CM544 dihydrochloride, and any combination thereof.

A composition for use in treating intestinal adenoma, wherein the composition comprises an amount of at least one chemical NOS inhibitor, wherein the at least one chemical NOS inhibitor is selected from the group consisting of 7-nitroindazole (“7-NI”), a salt of 7-NI, 3-bromo-7-nitroindazole (“3-Br-7-NI”), a salt of 3-Br-7-NI, N^{ω}-propyl-L-arginine (“L-NPA”), L-NPA hydrochloride, N⁵-(1-iminoethyl)-L-ornithine (“L-NIO”), L-NIO dihydrochloride, N-[4-[2-[[(3-chlorophenyl)methyl]amino]ethyl]phenyl]-2-thiophenecarboximidamide (“ARL-17477”), ARL-17477 dihydrochloride, N^{ω}-nitro-L-arginine (“L-NNA”), N^{ω}-nitro-L-arginine methyl ester (“L-NAME”), L-NAME hydrochloride, N^{ω}-methyl-L-arginine (“L-NMMA”), L-NMMA acetate, S-[2-[(1-iminoethyl)amino]ethyl]-L-homocysteine (“GW-274150”), GW-274150 phosphate, N⁶-(1-iminoethyl)-L-lysine (“L-NIL”), L-NIL hydrochloride, N-[[3-(aminomethyl)phenyl]methyl]-ethanimidamide (“1400W”), 1400W dihydrochloride, 2,4-disulfophenyl-N-tert-butyl nitrone (“OKN-007”), OKN-007 sodium, 2-[2-(4-methoxypyridin-2-yl)ethyl]-1H-imidazo[4,5-b]pyridine (“BYK-191023”), BYK-191023 dihydrochloride, N-{3-[(ethanimidoylamino)methyl]benzyl}-L-prolinamide (“CM544”), CM544 dihydrochloride, and any combination thereof.

A composition for use in treating sarcoma, wherein the composition comprises an amount of at least one chemical NOS inhibitor, wherein the at least one chemical NOS inhibitor is selected from the group consisting of 7-nitroindazole (“7-NI”), a salt of 7-NI, 3-bromo-7-nitroindazole (“3-Br-7-NI”), a salt of 3-Br-7-NI, N^{ω}-propyl-L-arginine (“L-NPA”), L-NPA hydrochloride, N⁵-(1-iminoethyl)-L-ornithine (“L-NIO”), L-NIO dihydrochloride, N-[4-[2-[[(3-chlorophenyl)methyl]amino]ethyl]phenyl]-2-thiophenecarboximidamide (“ARL-17477”), ARL-17477 dihydrochloride, N^{ω}-nitro-L-arginine (“L-NNA”), N^{ω}-nitro-L-arginine methyl ester (“L-NAME”), L-NAME hydrochloride, N^{ω}-methyl-L-arginine (“L-NMMA”), L-NMMA acetate, S-[2-[(1-iminoethyl)amino]ethyl]-L-homocysteine (“GW-274150”), GW-274150 phosphate, N⁶-(1-iminoethyl)-L-lysine (“L-NIL”), L-NIL hydrochloride, N-[[3-(aminomethyl)phenyl]methyl]-ethanimidamide (“1400W”), 1400W dihydrochloride, 2,4-disulfophenyl-N-tert-butyl nitrone (“OKN-007”), OKN-007 sodium, 2-[2-(4-methoxypyridin-2-yl)ethyl]-1H-imidazo[4,5-b]pyridine (“BYK-191023”), BYK-191023 dihydrochloride, N-{3-[(ethanimidoylamino)methyl]benzyl}-L-prolinamide (“CM544”), CM544 dihydrochloride, and any combination thereof.

A composition for use in treating ovarian cancer, wherein the composition comprises an amount of at least one chemical NOS inhibitor, wherein the at least one chemical NOS inhibitor is selected from the group consisting of 7-nitroindazole (“7-NI”), a salt of 7-NI, 3-bromo-7-nitroindazole (“3-Br-7-NI”), a salt of 3-Br-7-NI, N^{ω}-propyl-L-arginine (“L-NPA”), L-NPA hydrochloride, N⁵-(1-iminoethyl)-L-ornithine (“L-NIO”), L-NIO dihydrochloride, N-[4-[2-[[(3-chlorophenyl)methyl]amino]ethyl]phenyl]-2-thiophenecarboximidamide (“ARL-17477”), ARL-17477 dihydrochloride, N^{ω}-nitro-L-arginine (“L-NNA”), N^{ω}-nitro-L-arginine methyl ester (“L-NAME”), L-NAME hydrochloride, N^{ω}-methyl-L-arginine (“L-NMMA”), L-NMMA acetate, S-[2-[(1-iminoethyl)amino]ethyl]-L-homocysteine (“GW-274150”), GW-274150 phosphate, N⁶-(1-iminoethyl)-L-lysine (“L-NIL”), L-NIL hydrochloride, N-[[3-(aminomethyl)phenyl]methyl]-ethanimidamide (“1400W”), 1400W dihydrochloride, 2,4-disulfophenyl-N-tert-butyl nitrone (“OKN-007”), OKN-007 sodium, 2-[2-(4-methoxypyridin-2-yl)ethyl]-1H-imidazo[4,5-b]pyridine (“BYK-191023”), BYK-191023 dihydrochloride, N-{3-[(ethanimidoylamino)methyl]benzyl}-L-prolinamide (“CM544”), CM544 dihydrochloride, and any combination thereof.

A composition for use in treating cervical cancer, wherein the composition comprises an amount of at least one chemical NOS inhibitor, wherein the at least one chemical NOS inhibitor is selected from the group consisting of 7-nitroindazole (“7-NI”), a salt of 7-NI, 3-bromo-7-nitroindazole (“3-Br-7-NI”), a salt of 3-Br-7-NI, N^{ω}-propyl-L-arginine (“L-NPA”), L-NPA hydrochloride, N⁵-(1-iminoethyl)-L-ornithine (“L-NIO”), L-NIO dihydrochloride, N-[4-[2-[[(3-chlorophenyl)methyl]amino]ethyl]phenyl]-2-thiophenecarboximidamide (“ARL-17477”), ARL-17477 dihydrochloride, N^{ω}-nitro-L-arginine (“L-NNA”), N^{ω}-nitro-L-arginine methyl ester (“L-NAME”), L-NAME hydrochloride, N^{ω}-methyl-L-arginine (“L-NMMA”), L-NMMA acetate, S-[2-[(1-iminoethyl)amino]ethyl]-L-homocysteine (“GW-274150”), GW-274150 phosphate, N⁶-(1-iminoethyl)-L-lysine (“L-NIL”), L-NIL hydrochloride, N-[[3-(aminomethyl)phenyl]methyl]-ethanimidamide (“1400W”), 1400W dihydrochloride, 2,4-disulfophenyl-N-tert-butyl nitrone (“OKN-007”), OKN-007 sodium, 2-[2-(4-methoxypyridin-2-yl)ethyl]-1H-imidazo[4,5-b]pyridine (“BYK-191023”), BYK-191023 dihydrochloride, N-{3-[(ethanimidoylamino)methyl]benzyl}-L-prolinamide (“CM544”), CM544 dihydrochloride, and any combination thereof.

A composition for use in treating lung cancer associated with an upregulation of NO signaling, wherein the composition comprises an amount of at least one chemical NOS inhibitor, wherein the at least one chemical NOS inhibitor is selected from the group consisting of

A composition for use in treating non-small cell lung cancer, wherein the composition comprises an amount of at least one chemical NOS inhibitor, wherein the at least one chemical NOS inhibitor is selected from the group consisting of 7-nitroindazole (“7-NI”), a salt of 7-NI, 3-bromo-7-nitroindazole (“3-Br-7-NI”), a salt of 3-Br-7-NI, N^{ω}-propyl-L-arginine (“L-NPA”), L-NPA hydrochloride, N⁵-(1-iminoethyl)-L-ornithine (“L-NIO”), L-NIO dihydrochloride, N-[4-[2-[[(3-chlorophenyl)methyl]amino]ethyl]phenyl]-2-thiophenecarboximidamide (“ARL-17477”), ARL-17477 dihydrochloride, N^{ω}-nitro-L-arginine (“L-NNA”), N^{ω}-nitro-L-arginine methyl ester (“L-NAME”), L-NAME hydrochloride, N^{ω}-methyl-L-arginine (“L-NMMA”), L-NMMA acetate, S-[2-[(1-iminoethyl)amino]ethyl]-L-homocysteine (“GW-274150”), GW-274150 phosphate, N⁶-(1-iminoethyl)-L-lysine (“L-NIL”), L-NIL hydrochloride, N-[[3-(aminomethyl)phenyl]methyl]-ethanimidamide (“1400W”), 1400W dihydrochloride, 2,4-disulfophenyl-N-tert-butyl nitrone (“OKN-007”), OKN-007 sodium, 2-[2-(4-methoxypyridin-2-yl)ethyl]-1H-imidazo[4,5-b]pyridine (“BYK-191023”), BYK-191023 dihydrochloride, N-{3-[(ethanimidoylamino)methyl]benzyl}-L-prolinamide (“CM544”), CM544 dihydrochloride, and any combination thereof.

A composition for use in treating metastatic squamous cell carcinoma of the head and neck, wherein the composition comprises an amount of at least one chemical NOS inhibitor, wherein the at least one chemical NOS inhibitor is selected from the group consisting of 7-nitroindazole (“7-NI”), a salt of 7-NI, 3-bromo-7-nitroindazole (“3-Br-7-NI”), a salt of 3-Br-7-NI, N^{ω})-propyl-L-arginine (“L-NPA”), L-NPA hydrochloride, N⁵-(1-iminoethyl)-L-ornithine (“L-NIO”), L-NIO dihydrochloride, N-[4-[2-[[(3-chlorophenyl)methyl]amino]ethyl]phenyl]-2-thiophenecarboximidamide (“ARL-17477”), ARL-17477 dihydrochloride, N^{ω}-nitro-L-arginine (“L-NNA”), N^{ω}-nitro-L-arginine methyl ester (“L-NAME”), L-NAME hydrochloride, N^{ω}-methyl-L-arginine (“L-NMMA”), L-NMMA acetate, S-[2-1[(1-iminoethyl)amino]ethyl]-L-homocysteine (“GW-274150”), GW-274150 phosphate, N⁶-(1-iminoethyl)-L-lysine (“L-NIL”), L-NIL hydrochloride, N-[[3-(aminomethyl)phenyl]methyl]-ethanimidamide (“1400W”), 1400W dihydrochloride, 2,4-disulfophenyl-N-tert-butyl nitrone (“OKN-007”), OKN-007 sodium, 2-[2-(4-methoxypyridin-2-yl)ethyl]-1H-imidazo[4,5-b]pyridine (“BYK-191023”), BYK-191023 dihydrochloride, N-{3-[(ethanimidoylamino)methyl]benzyl}-L-prolinamide (“CM544”), CM544 dihydrochloride, and any combination thereof.

A composition for use in treating squamous cell carcinoma of the skin, wherein the composition comprises an amount of at least one chemical NOS inhibitor, wherein the at least one chemical NOS inhibitor is selected from the group consisting of 7-nitroindazole (“7-NI”), a salt of 7-NI, 3-bromo-7-nitroindazole (“3-Br-7-NI”), a salt of 3-Br-7-NI, N^{ω}-propyl-L-arginine (“L-NPA”), L-NPA hydrochloride, N⁵-(1-iminoethyl)-L-ornithine (“L-NIO”), L-NIO dihydrochloride, N-[4-[2-[[(3-chlorophenyl)methyl]amino]ethyl]phenyl]-2-thiophenecarboximidamide (“ARL-17477”), ARL-17477 dihydrochloride, N^{ω}-nitro-L-arginine (“L-NNA”), N^{ω}-nitro-L-arginine methyl ester (“L-NAME”), L-NAME hydrochloride, N^{ω}-methyl-L-arginine (“L-NMMA”), L-NMMA acetate, S-[2-[(1-iminoethyl)amino]ethyl]-L-homocysteine (“GW-274150”), GW-274150 phosphate, N⁶-(1-iminoethyl)-L-lysine (“L-NIL”), L-NIL hydrochloride, N-[[3-(aminomethyl)phenyl]methyl]-ethanimidamide (“1400W”), 1400W dihydrochloride, 2,4-disulfophenyl-N-tert-butyl nitrone (“OKN-007”), OKN-007 sodium, 2-[2-(4-methoxypyridin-2-yl)ethyl]-1H-imidazo[4,5-b]pyridine (“BYK-191023”), BYK-191023 dihydrochloride, N-{3-[(ethanimidoylamino)methyl]benzyl}-L-prolinamide (“CM544”), CM544 dihydrochloride, and any combination thereof.

While exemplary embodiments, aspects and variations have been provided herein, those of skill in the art will recognize certain modifications, permutations, additions and combinations and certain sub-combinations of the embodiments, aspects, and variations. It is intended that the following claims are interpreted to include all such modifications, permutations, additions and combinations and certain sub-combinations of the embodiments, aspects and variations are within their scope.

### Examples

### Example 1: 7-NI—Neuroblastoma

### Methods

### SH-SY5Y Cell Cultures and Treatment

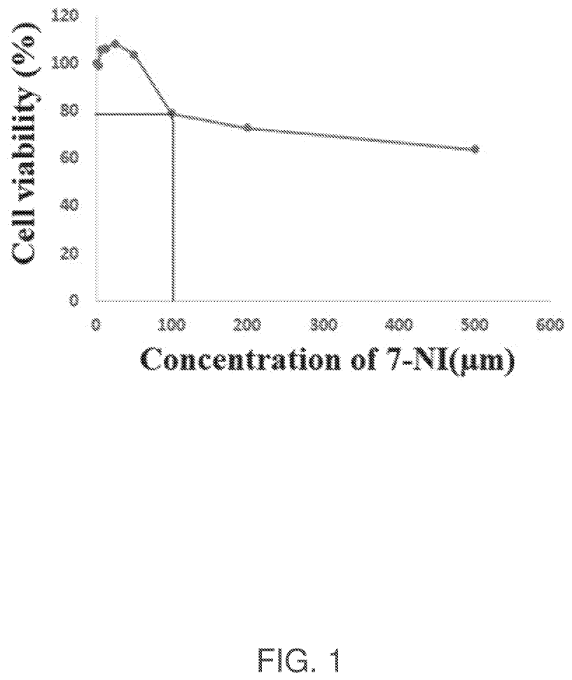

Human neuroblastoma (“NB”) SH-SY5Y cells were obtained from the American Type Culture Collection (ATCC) and maintained in Dulbecco's modified Eagle's medium (DMEM) and Ham's F-12 (1:1) supplemented with 10% fetal bovine serum, 2 mM L-glutamine, and 100 U/ml penicillin-G/100 mg/ml streptomycin in the humidified atmosphere (37° C., 5% CO₂). Cells were plated onto 25 cm² and 75 cm² at a density of 7.0×10⁵ and 2.1×10⁶ cells/cm² respectively for Western blotting (WB) and 1×10⁵ for immunofluorescence. The SH-SY5Y cells were treated with 100 μM 7-nitroindazole (7-NI) for 24 hr. This concentration was confirmed in the dose-response experiment (FIG. 1) using an MTT viability assay.

### MTT Cell Viability Assay

Effects of 7-NI on neuroblastoma cell growth were determined using a colorimetric 3-(4,5-dimethylthiazol-2-yl)-2,5-diphenyl-tetrazoleum (MTT) assay (Sigma-Aldrich). Five or six parallels of each treatment were performed in each experiment.

### Clonogenic Survival Assay

Cells were seeded into 6-well plates at concentrations of 500/well or 1000/well in triplicates. After 24 hours, 7-NI was added, or cells were transiently transfected with siRNA-nNOS, as described above (SEQ ID NO: 4), before changing to serum-containing medium. Cells were incubated for 14 days to form visible colonies. Colonies were stained with crystal violet, washed with PBS, air-dried, and imaged. The colonies were visualized using ImageJ software. Cell colonies of greater than 50 cells were identified and counted.

### Measurement of NADPH-Diaphorase Activity

The NADPH-diaphorase activity assay was used to determine the NOS activity based on the NADPH-dependent reduction of nitro-blue tetrazolium salt (NBT) to visible formazan. SH-SYSY cells grown in six-well culture plates were lysed in 50 mM Tris-HCl buffer (pH 7.4), containing 0.1% Triton X-100, 2 mM EDTA, and a protease inhibitor cocktail (Sigma, St. Louis, MO). 100 μL of the lysate were added to 250 μL of 50 mM Tris-HCl, pH 7.5, containing 5 mM EDTA, 0.5 mM NBT and 0.5 mM NADPH. After a 15 min incubation period at 37° C., the production of formazan was measured spectrophotometrically at 585 nm. The data were expressed as absorbance units and normalized for the protein content of each sample. Protein concentration was measured by the bicinchoninic acid (BCA) protein assay using BSA as a standard.

### NADPH-Diaphorase Staining

NADPH diaphorase staining were done using NBT as a chromogen and NADPH enzyme as a reductant to detect cells with NOS activity. Cells were cultured and treated as described previously in the immunofluorescence section. Then cells were fixed with 4% PFA for 30 min at room temperature in dark. After fixation, the cells were washed 2 times with PBS and then incubated in 0.2% Triton X-100 (in phosphate buffer) for 10 min. The cells were then incubated in 1 of NBT-NADPH solution for 2 h at 37C. The cells were washed twice with PBS and permanently mounted with DPX. Images were analyzed with a Nikon confocal TL microscope.

### Nitrite Assay

Released NO was measured with Griess assay Kit (Sigma-Aldrich, Cat. No. 23479) using the manufacturer's instructions.

### Western Blot (WB)

Cells were lysed in ice-cold RIPA buffer containing 50 mM Tris-HCl, 1% Nonidet P-40, 0.25% sodium deoxycholate, 150 mM NaCl, protease-, and phosphatase inhibitor cocktails, pH 7.4. After sonication, the lysates were centrifuged at 17,000 g for 30 min at 4° C., and the supernatant was collected. The protein concentrations were determined with the BCA protein assay. Proteins were resolved by SDS-PAGE, transferred to a PVDF membrane, and probed with the following antibodies: anti-pMTOR (rabbit, diluted 1:1000), anti-mTOR (rabbit, diluted 1:1000), anti-TSC2 (rabbit, diluted 1:1000), anti-nNOS (rabbit, diluted 1:1000), anti-pAKT (rabbit, diluted 1:1000), anti-AKT (rabbit, diluted 1: 1000), anti-pRPS6 (rabbit, diluted 1:1000), anti-RPS6 (mouse, diluted 1:1000), anti-synaptophysin (mouse, diluted 1:1000), anti-β-actin (rabbit, diluted 1:1000), anti-3-Nitrotyrosine (rabbit, diluted 1:5000) from Abcam. All primary antibodies, except anti-3-Nitrotyrosine, were purchased from Cell Signaling Technology. The blots were then visualized using an enhanced chemiluminescence (ECL) system.

### Confocal Microscopy

Cells cultured on coverslips coated with Poly-D-Lysine were washed three times with PBS and fixed in 4% paraformaldehyde for 15-20 min at room temperature. The fixed cells were washed three times with PBS and incubated in the presence of permeabilization buffer (PBS containing 0.1% Triton X-100) for 5 min at room temperature. After washing three times with PBS, the cells were blocked with blocking buffer (PBS containing 1% BSA, 1% goat serum, and 0.1% tritonX-100, pH 7.4) for 30 min at room temperature and then incubated overnight at 4° C. with the following antibodies: anti-nNOS (rabbit, diluted 1:200), anti-MAP2 (mouse/rabbit, diluted 1:200), anti-synaptophysin (rabbit, diluted 1:200), anti-3-Nitrotyrosine (mouse, diluted 1:200) from Abcam. All primary antibodies, except anti-3-Nitrotyrosine, were purchased from Cell Signaling Technology. Then, the samples were incubated with Alexa 488—or Alexa 594-conjugated secondary antibodies for 2 h and then mounted with DAPI-containing mounting media. The mounted slides were observed under a confocal microscope.

### In Vivo Animal Experiments

A xenograft neuroblastoma model was generated by injecting 4.0×10⁶ SH-SY5Y cells (in a 50:50 mixture of PBS and matrigel basement membrane matrix) into the subcutaneous tissue in the flank of NOD SCID Gamma (NSG) male mice. Mice were 6-8 weeks old at time of injections. The mice were examined for 1 month.

Two treatment groups of mice were used (n=8), wherein one group was administered control, and the second group was administered 80 mg/kg of 7-NI administered intraperitoneally three times per week. Treatment started after five days from the injection with SH-SY5Y cells. After one month, all mice were euthanized, and tumors were harvested for ex vivo analysis and histology. Tumor volume was measured using calipers and was calculated as (length\hat{ }2*width)/2

### Statistical Analysis

Each experiment was repeated at least three times, with a satisfactory correlation achieved between the results of individual experiments. The data shown are the average of 3-4 culture dishes for each group. In each experiment, differences between groups were assessed using the one-way ANOVA followed by Fisher's LSD multiple comparison post hoc test, with values of P<0.05 considered to indicate statistical significance. Data are expressed as the mean±SEM.

### Results

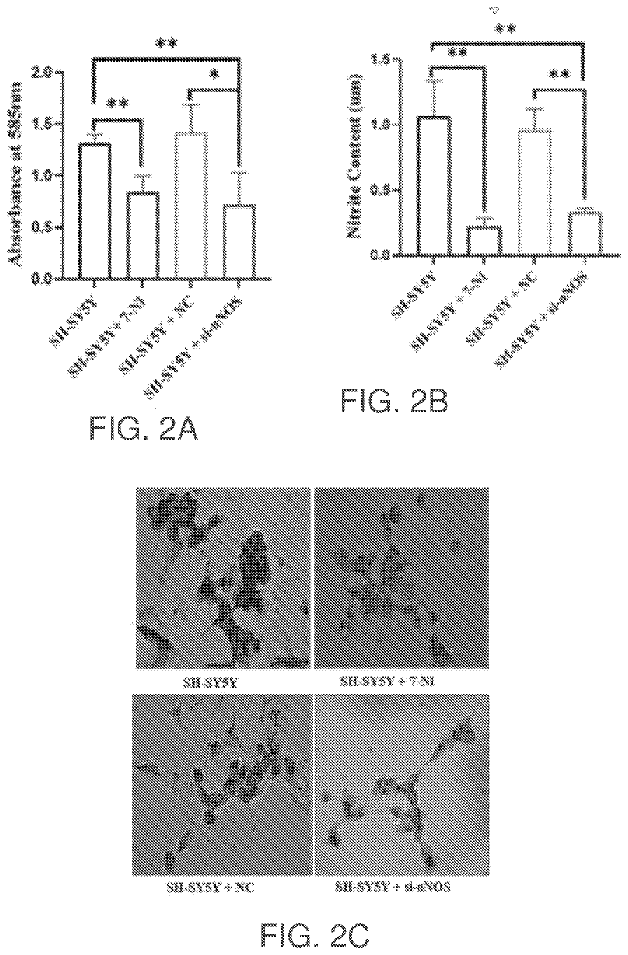

SH-SY5Y cells displayed high levels of NADPH diaphorase activity showing high NOS activity, which was significantly inhibited by the treatment with 7-NI (FIG. 2A). NADPH diaphorase staining were also found decreased in the 7-NI treated group compared to the untreated group (FIG. 2C). The increased NOS activity may also lead to increased production of NO in SH-SY5Y cells. The increased NO is showed through its stable product formation and measured as nitrite content. The Griess assay showed the dramatic reduction of nitrite content in SH-SY5Y cells treated with 7-NI. The results shows, treatment with 7-NI of SH-SY5Y cells inhibits both NADPH Diaphorase activity as well as NO production, which may be indicative of global nNOS activity.

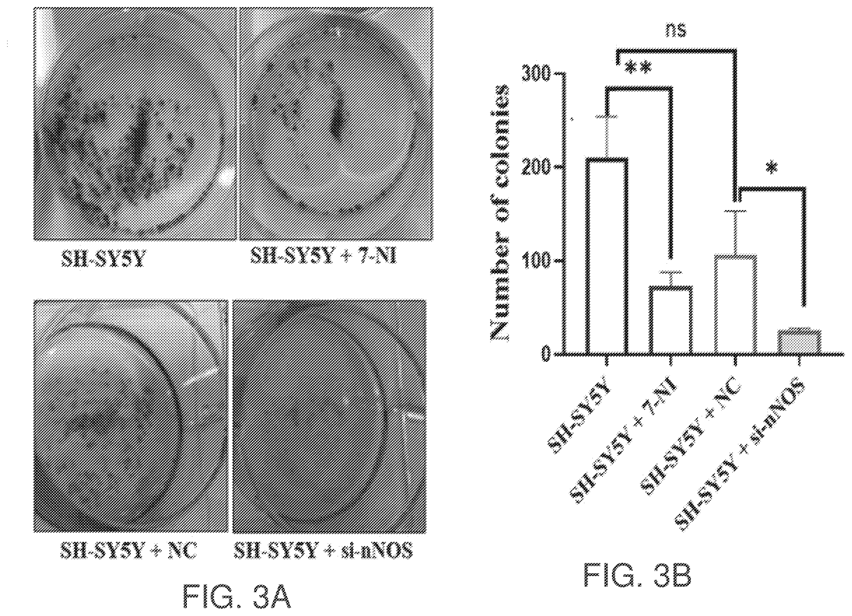

The clonogenic survival assay were done to assess the role of increased production of NO in proliferation of SH-SY5Y cell line. The results showed a decrease in the number of colonies following the 7-NI treatment in SH-SY5Y cells (FIGS. 3A-B). This demonstrates a reduced proliferative capacity of the SH-SY5Y cells after reduction in NO production.

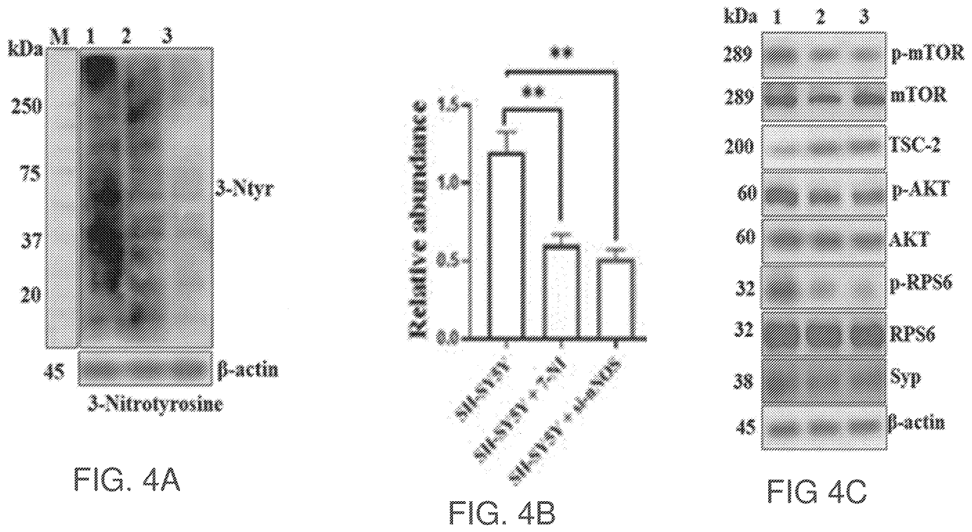

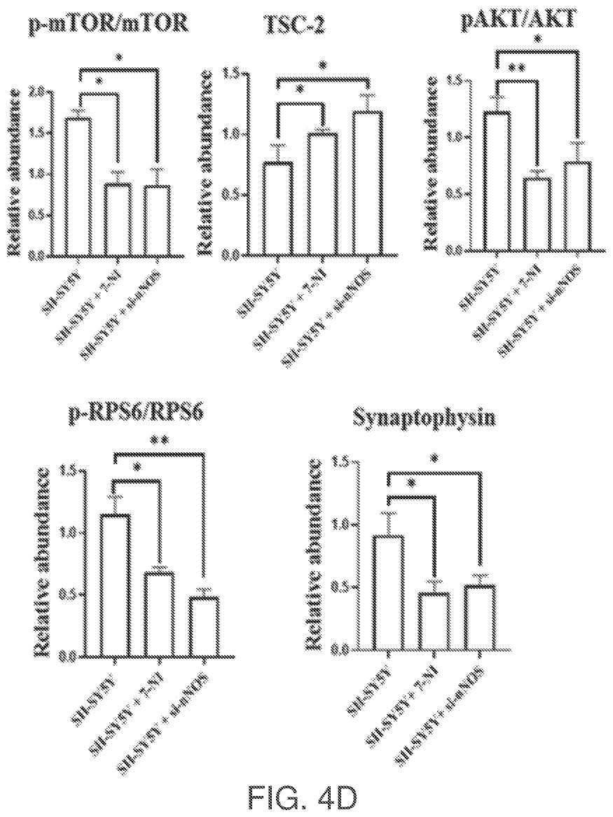

Nitrotyrosine is a stable NO end product and marker of increased NO production. To assess the contribution of nNOS to human neuroblastoma growth, the SH-SY5Y human neuroblastoma cell line was treated with nNOS selective inhibitor (7-NI). Treatment of SH-SY5Y cells with 7-NI halved 3-Ntyr levels (FIGS. 4A-B). Since the mTOR pathway has been shown to be a key mediator of proliferation and survival for neuroblastoma and other tumors, the mTOR pathway was a study point of emphasis for SH-SY5Y cells. Treatment of SH-SY5Y cells with 7-NI led to a significant increase in the levels of TSC-2, a key negative regulator of mTORC1 signaling pathway, and resulted in a decrease in phosphorylation of mTOR (FIG. 4D). Phosphorylation of an upstream component of this signaling pathway, protein kinase-B (AKT), and its downstream target, RPS6 was also reduced (FIGS. 4C-D). Additionally, 7-NI significantly decreased the levels of synaptophysin (FIGS. 4C-D). Without being bound by any particular theory, upon treatment with 7-NI, the increase in TSC2 expression could be associated with the reduced S-nitrosylation and consequently, reduced degradation of this protein. The increase in TSC2 levels was accompanied by the reduction in phosphorylation of the key proteins of the mTOR signaling pathway, the upstream activator of mTORC1 signaling AKT, mTOR itself, and its core substrate RPS6, indicating the reduced activity of the mTOR signaling pathway. Remarkably, these results show that inhibition of nNOS, by 7-NI, significantly increased the levels of TSC2, which may disrupt activation of the mTOR pathway that is believed to be involved in the stabilization of MYCN protein, which may support neuroblastoma growth and proliferation.

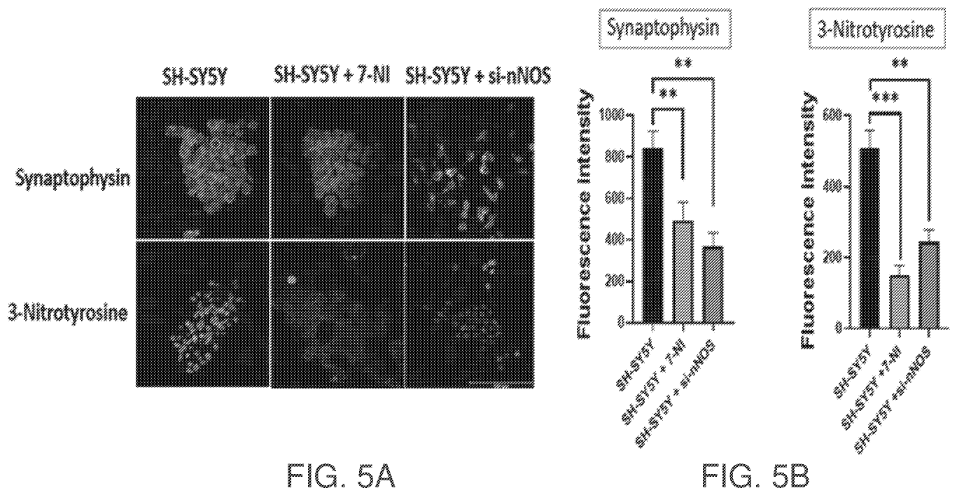

Synaptophysin is expressed independently of other neuronal differentiation markers and propose that it be used as a differentiation marker in tumor diagnosis. SH-SY5Y cells showed a relatively high intensity of 3-Ntyr and Syp immunofluorescence. Pharmacological inhibition 7-NI significantly decreased the fluorescence intensity of 3-Ntyr and Syp (FIGS. 5A-C).

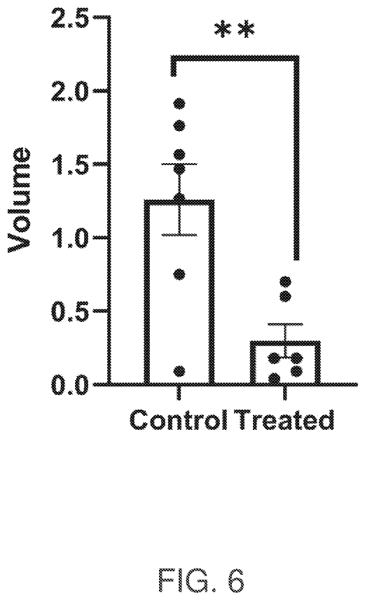

Following treatment with 7-NI, mice showed a significantly smaller average tumor size (FIG. 6).

NO takes part in the regulation of many physiological and pathological functions, which may include the development of glioblastoma and/or neuroblastoma. The results presented herein show that the inhibition of NO production in SH-SY5Y cells (a human neuroblastoma cell line), as well as the inhibition of tumor growth in SH-SY5Y-xenografted mice by pharmacological intervention with the selective nNOS inhibitor 7-NI can effectively suppress the neuroblastoma proliferation and growth, evidenced by significantly reduced levels of the marker of Syp and the nitrosative stress indicator, 3-Ntyr, as well as reduced tumor growth in vivo, compared to the control.

Without being bound by any particular theory, it is believed that NO production may play a significant role in the development and progression of glioblastoma and/or neuroblastoma, among others. The effects of NO production may depend on NO concentration, exposure time, tumor type, microenvironment, and the like, however, high levels of NO (O(μM)) are suspected to cause DNA damage, and researchers have found that overexpression of NOS isoforms and excessive NO levels may play a role in the development and progression of glioblastoma and/or neuroblastoma, and may even hinder the efficacy of conventional therapies used to treat these malignancies. It is believed that inflammation can induce NO-related carcinogenesis via induction of DNA damage, suppression of DNA repair enzymes, and PTMs including SNO, because during inflammation, reactive nitrogen and oxygen species are released by activated inflammatory cells, attacking neighboring epithelial and stromal cells, altering function, and initiating carcinogenesis. The events leading to the transition to a more aggressive tumor phenotype include activation or upregulation of pro-survival/pro-expansion effector proteins, such as, for example, NF-κB, phosphoinositide-3-kinase (PI3K), AKT, p300, among others.

Collectively, the results of this study suggest that inhibition of nNOS in a human neuroblastoma model (i.e. SH-SY5Y cells), both in vitro and in vivo, may suppress the malignancy of these cells via reduced activity of the mTOR signaling system. Because nNOS appears to be an important mediator of neuroblastoma survival, proliferation, and carcinogenesis, suppression of nNOS using the novel NOS inhibitor compositions disclosed herein, e.g., 7-NI, may have a vast utility in treating, ameliorating, and/or reducing the severity of glioblastoma and/or neuroblastoma, as well as others cancer where overexpression of nNOS is implicated, e.g., gastrointestinal cancers, sarcomas, ovarian cancers, cervical cancers, and the like. Moreover, because the other NOS isoforms, i.e., iNOS and eNOS, impart similar physiological effects, i.e., production of aberrant NO, the NOS inhibitor compositions disclosed herein, e.g., 7-NI, may be viable therapeutic options for pathologies wherein iNOS and/or eNOS are implicated, e.g., gastrointestinal cancers, pancreatic cancers, sarcomas, ovarian cancers, cervical cancers, lung cancers, melanomas, cancers of the head and neck and the like.

### Example 2: siRNA—Neuroblastoma

### Methods

The same methods that were utilized in Example 1 were utilized in Example 2. The difference in Example 2 compared to Example 1 is that siRNA was administered instead of 7-NI.

siRNA transfection

SH-SY5Y cells were plated in 6 well plates, as well as on 22 mm² coverslips at a density of 6.0×10⁴ cells/cm² and transfected with 20 nM nNOS siRNA (SEQ ID NO: 4) for 24 h, with the use of Lipofectamine RNAi MAX (56530, Invitrogen) according to the manufacturer's protocol. Then cells were washed with phosphate-buffered saline (PBS) and harvested for WB and fixed for immunofluorescence.

### Results

SH-SY5Y cells displayed high levels of NADPH diaphorase activity showing high NOS activity, which was significantly inhibited by the treatment with nNOS knockdown (siRNA). NADPH diaphorase staining were also found decreased in the nNOS knockdown group as compared to untreated SH-SY5Y cells (FIG. 2C). The increased NOS activity may also lead to increased production of NO in SH-SY5Y cells. The increased NO is showed through its stable product formation and measured as nitrite content. The Griess assay showed the dramatic reduction of nitrite content in SH-SY5Y cells following the nNOS silencing (FIG. 2B). The results shows, treatment with nNOS knockdown of SH-SY5Y cells inhibits both NADPH Diaphorase activity as well as NO production, which may be indicative of global nNOS activity.

The clonogenic survival assay were done to assess the role of increased production of NO in proliferation of SH-SY5Y cell line. The results showed a decrease in the number of colonies following the nNOS silencing in SH-SY5Y cells (FIGS. 3A-B). This demonstrates a reduced proliferative capacity of the SH-SY5Y cells after reduction in NO production.

Nitrotyrosine is a stable NO end product and marker of increased NO production. To assess the contribution of nNOS to human neuroblastoma growth, the SH-SY5Y human neuroblastoma cell line was treated with nNOS-targeting small interfering RNA (siRNAs). Treatment of SH-SY5Y cells with siRNAs halved 3-Ntyr levels (FIGS. 4A-B). Since the mTOR pathway has been shown to be a key mediator of proliferation and survival for neuroblastoma and other tumors, the mTOR pathway was a study point of emphasis for SH-SY5Y cells. Treatment of SH-SY5Y cells with nNOS-specific siRNAs led to a significant increase in the levels of TSC-2, a key negative regulator of mTORC1 signaling pathway, and resulted in a decrease in phosphorylation of mTOR (FIG. 4D). Phosphorylation of an upstream component of this signaling pathway, protein kinase-B (AKT), and its downstream target, RPS6 was also reduced (FIGS. 4C-D). Additionally, treatment with siRNAs significantly decreased the levels of synaptophysin (FIGS. 4C-D).

Synaptophysin is expressed independently of other neuronal differentiation markers and propose that it be used as a differentiation marker in tumor diagnosis. SH-SY5Y cells showed a relatively high intensity of 3-Ntyr and Syp immunofluorescence. Knockdown of nNOS with siRNAs significantly decreased the fluorescence intensity of 3-Ntyr and Syp (FIGS. 5A-D).

Without being bound by any particular theory, upon treatment with siRNAs, the increase in TSC2 expression could be associated with the reduced S-nitrosylation and consequently, reduced degradation of this protein. The increase in TSC2 levels was accompanied by the reduction in phosphorylation of the key proteins of the mTOR signaling pathway, the upstream activator of mTORC1 signaling AKT, mTOR itself, and its core substrate RPS6, indicating the reduced activity of the mTOR signaling pathway. Remarkably, these results show that inhibition of nNOS, by siRNAs, significantly increased the levels of TSC2, which may disrupt activation of the mTOR pathway that is believed to be involved in the stabilization of MYCN protein, which may support neuroblastoma growth and proliferation.

As discussed above, because NO takes part in the regulation of many physiological and pathological functions, which may include the development of glioblastoma and/or neuroblastoma. The results presented herein show that the inhibition of NO production in SH-SY5Y cells (a human neuroblastoma cell line) by pharmacological intervention with the nNOS-specific siRNAs can effectively suppress the neuroblastoma proliferation and growth, evidenced by significantly reduced levels of the marker of Syp and the nitrosative stress indicator, 3-Ntyr.

Without being bound by any particular theory, it is believed that NO production may play a significant role in the development and progression of glioblastoma and/or neuroblastoma, among others. The effects of NO production may depend on NO concentration, exposure time, tumor type, microenvironment, and the like, however, high levels of NO (O(μM)) are suspected to cause DNA damage, and researchers have found that overexpression of NOS isoforms and excessive NO levels may play a role in the development and progression of glioblastoma and/or neuroblastoma, and may even hinder the efficacy of conventional therapies used to treat these malignancies. It is believed that inflammation can induce NO-related carcinogenesis via induction of DNA damage, suppression of DNA repair enzymes, and PTMs including SNO, because during inflammation, reactive nitrogen and oxygen species are released by activated inflammatory cells, attacking neighboring epithelial and stromal cells, altering function, and initiating carcinogenesis. The events leading to the transition to a more aggressive tumor phenotype include activation or upregulation of pro-survival/pro-expansion effector proteins, such as, for example, NF-κB, phosphoinositide-3-kinase (PI3K), AKT, p300, among others.

Collectively, the results of this study suggest that inhibition of nNOS in a human neuroblastoma model (i.e., SH-SY5Y cells) in vitro may suppress the malignancy of these cells via reduced activity of the mTOR signaling system. Because nNOS appears to be an important mediator of neuroblastoma survival, proliferation, and carcinogenesis, suppression of nNOS using the novel NOS inhibitor compositions disclosed herein, e.g., nNOS-specific siRNAs, may have a vast utility in treating, ameliorating, and/or reducing the severity of glioblastoma and/or neuroblastoma, as well as others cancer where overexpression of nNOS is implicated, e.g., gastrointestinal cancers, sarcomas, ovarian cancers, cervical cancers, and the like. Moreover, because the other NOS isoforms, i.e., iNOS and eNOS, impart similar physiological effects, i.e., production of aberrant NO, the NOS inhibitor compositions disclosed herein, e.g., 7-NI, may be viable therapeutic options for pathologies wherein iNOS and/or eNOS are implicated, e.g., gastrointestinal cancers, pancreatic cancers, sarcomas, ovarian cancers, cervical cancers, lung cancers, melanomas, cancers of the head and neck, and the like.

### Example 3: 7-NI—Glioblastoma

### Methods

The same methods that were utilized in Example 1 were utilized in Example 2. The difference in Example 3 compared to Example 1 is that U87-MG cells were used for the in vitro work as well as for the in vivo xenograft. U87-MG cells were used to evaluate the efficacy of the NOS inhibitor compositions disclosed herein for glioblastoma.

### U87-MG Cell Cultures and Treatment

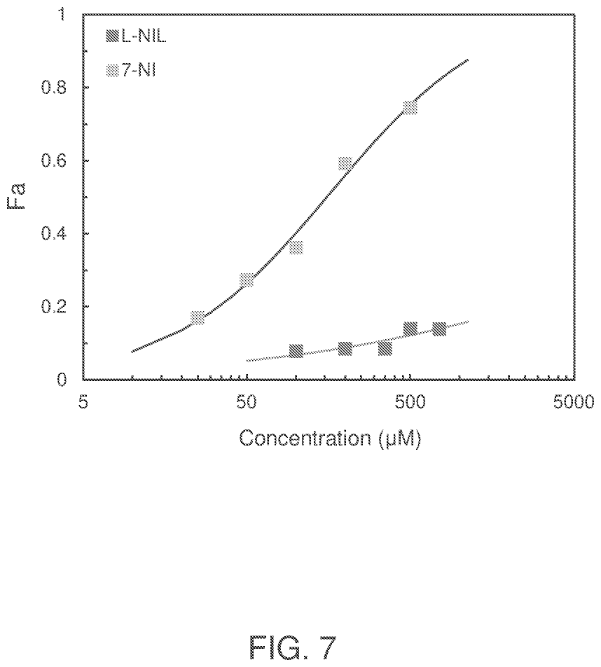

Human glioblastoma U87-MG cells were obtained from the American Type Culture Collection (ATCC) and maintained in Dulbecco's modified Eagle's Medium (DMEM) and Ham's F-12 (1:1) supplemented with 10% fetal bovine serum, 2 mM L-glutamine, and 100 U/ml penicillin-G/100 mg/ml streptomycin in the humidified atmosphere (37° C., 5% CO₂). Cells were plated onto 25 cm² and 75 cm² at a density of 7.0×10⁵ and 2.1×10⁶ cells/cm² respectively for Western blotting (WB) and 1×10⁵ for immunofluorescence. The U87-MG cells were treated with 100 μM 7-nitroindazole (7-NI) for 24 hr. This concentration was confirmed in the dose-response experiment (FIG. 7) using an MTT viability assay.

### MTT Cell Viability Assay

Effects of 7-NI on glioblastoma cell growth were determined using a colorimetric 3-(4,5-dimethylthiazol-2-yl)-2,5-diphenyl-tetrazoleum (MTT) assay (Sigma-Aldrich). The in vitro dose response curve for 7-NI is presented in FIG. 7.

### Clonogenic Survival Assay

Cells were seeded into 6-well plates at concentrations of 500/well or 1000/well in triplicates. After 24 hours, 7-NI was added, or cells were transiently transfected with siRNA-nNOS, as described above, before changing to serum-containing medium. Cells were incubated for 14 days to form visible colonies. Colonies were stained with crystal violet, washed with PBS, air-dried, and imaged. The colonies were visualized using ImageJ software. Cell colonies of greater than 50 cells were identified and counted.

### Nitrite Assay

Released NO was measured with Griess assay Kit (Sigma-Aldrich, Cat. No. 23479) using the manufacturer's instructions.

### Western Blot (WB)

Cells were lysed in ice-cold RIPA buffer containing 50 mM Tris-HCl, 1% Nonidet P-40, 0.25% sodium deoxycholate, 150 mM NaCl, protease-, and phosphatase inhibitor cocktails, pH 7.4. After sonication, the lysates were centrifuged at 17,000 g for 30 min at 4° C., and the supernatant was collected. The protein concentrations were determined with the BCA protein assay. Proteins were resolved by SDS-PAGE, transferred to a PVDF membrane, and probed with the following antibodies: anti-CD70 (rabbit, diluted 1:1000), and anti-3-Nitrotyrosine (rabbit, diluted 1:5000) from Abcam. All primary antibodies, except anti-3-Nitrotyrosine, were purchased from Cell Signaling Technology. The blots were then visualized using an enhanced chemiluminescence (ECL) system.

### In Vivo Animal Experiments

A xenograft glioblastoma model was generated by injecting 1.0×10⁶ U87-MG cells (in PBS) into the subcutaneous tissue in the flank of NOD SCID Gamma (NSG) male (n=40) and female mice (n=5). Mice were 6-7 weeks old at time of injections. Mice were divided into groups and treatment was started when tumor volume reached 50 mm³.

An initial experiment was performed with two treatment groups of mice, the treatment groups were: 0.1% DMSO (“Vehicle”) (n=10) and 80 mg/kg of 7-NI (n=10). Treatments were administered intraperitoneally daily for 8 days. Treatment started after five days from the injection with U87-MG cells. After one month, all mice were euthanized, and tumors were harvested for ex vivo analysis and histology. Tumor volume was measured using calipers and was calculated as (length*width\hat{ }2)/2. The tumor volume of all mice were tabulated, as well the best 4-5 mice in terms of treatment efficacy (smallest tumor volume).

A dose response experiment was performed in the mice, the treatment groups were 0.1% DMSO (“Vehicle”) (n=8), 20 mg/kg 7-NI (n=8), 40 mg/kg 7-NI (n=8), and 80 mg/kg 7-NI (n=8). Treatments were administered intraperitoneally daily, for 14 days. Treatment started after five days from the injection with U87-MG cells. All mice were euthanized, and tumors were harvested for ex vivo analysis and histology. Tumor volume was measured using calipers and was calculated as (length*width\hat{ }2)/2. The tumor volume of all mice were tabulated, as well the tumor mass.

A survival experiment was performed in the mice, the treatment groups were saline (“Vehicle”) (n=10), 80 mg/kg 7-NI (n=10), and 120 mg/kg 7-NI (n=8). Treatments were administered intraperitoneally daily.

### Statistical Analysis

Each experiment was repeated at least three times, with a satisfactory correlation achieved between the results of individual experiments. The data shown are the average of 3-4 culture dishes for each group. In each experiment, differences between groups were assessed using the one-way ANOVA followed by Fisher's LSD multiple comparison post hoc test, with values of P<0.05 considered to indicate statistical significance. Data are expressed as the mean±SEM.

### Results

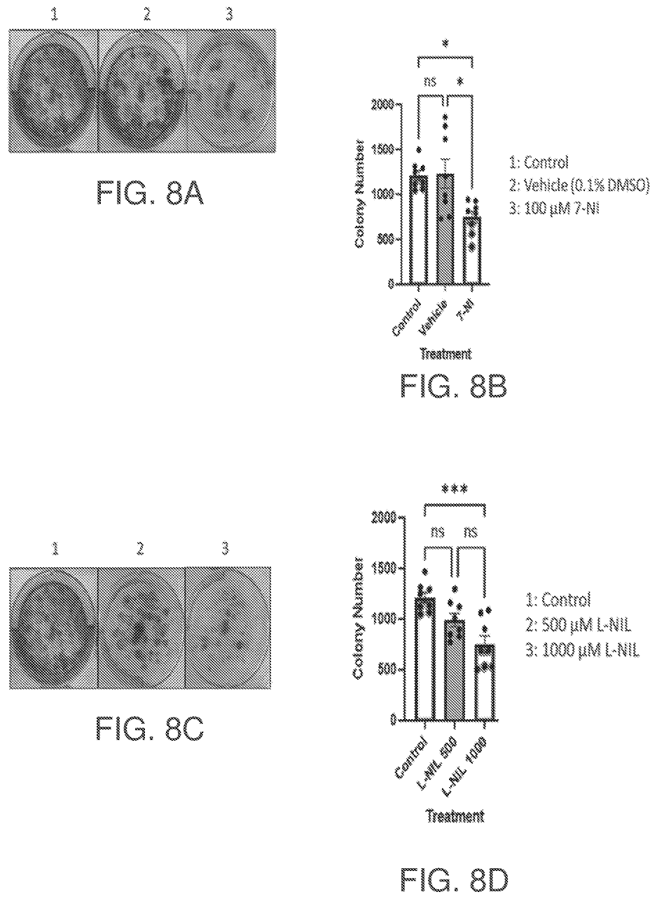

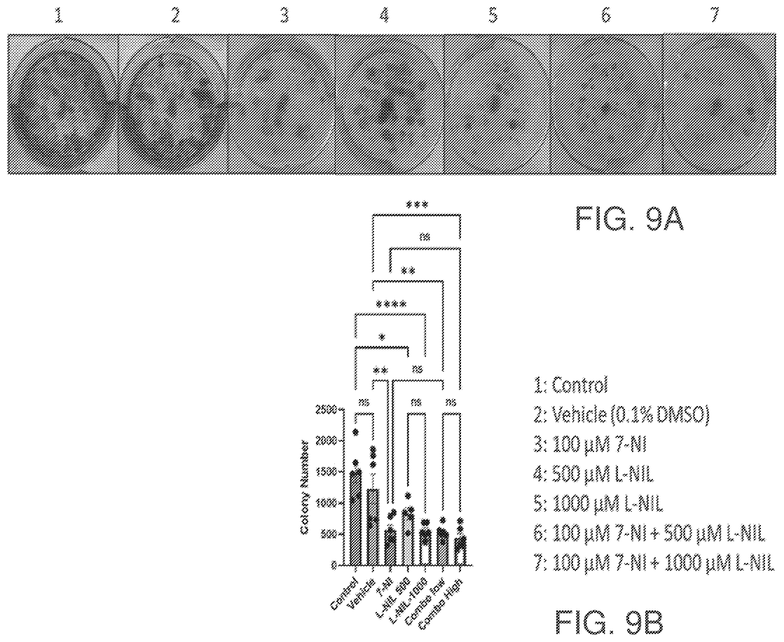

In the colony assay, 7-NI significantly impacted colony growth compared to both the control and the negative control (0.1% DMSO). Because 7-NI is an nNOS-specific inhibitor, these results indicate that the observed decrease in the number of colonies following treatment with 7-NI may occur via suppression of nNOS in U87-MG cells (FIGS. 8A-8B and 9).

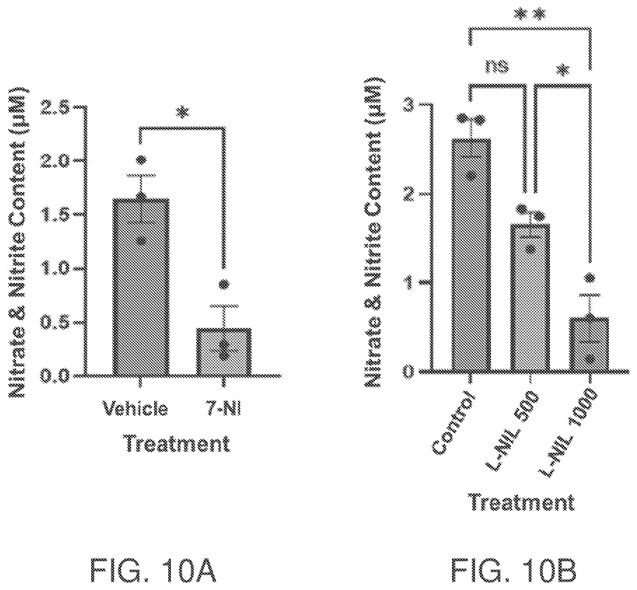

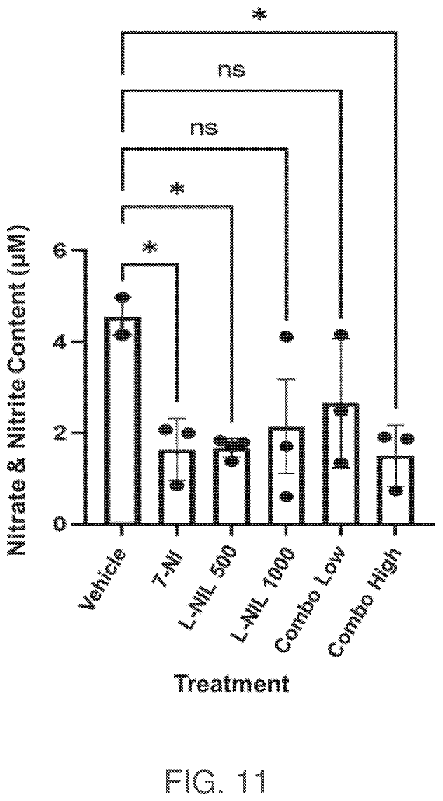

The Griess assay showed the dramatic reduction of nitrite and nitrate content in U87-MG cells following treatment with 7-NI (FIGS. 10A and 11). Because 7-NI is an nNOS-specific inhibitor, these results indicate that the observed decrease in nitrite and nitrate content may occur because of global nNOS activity suppression.

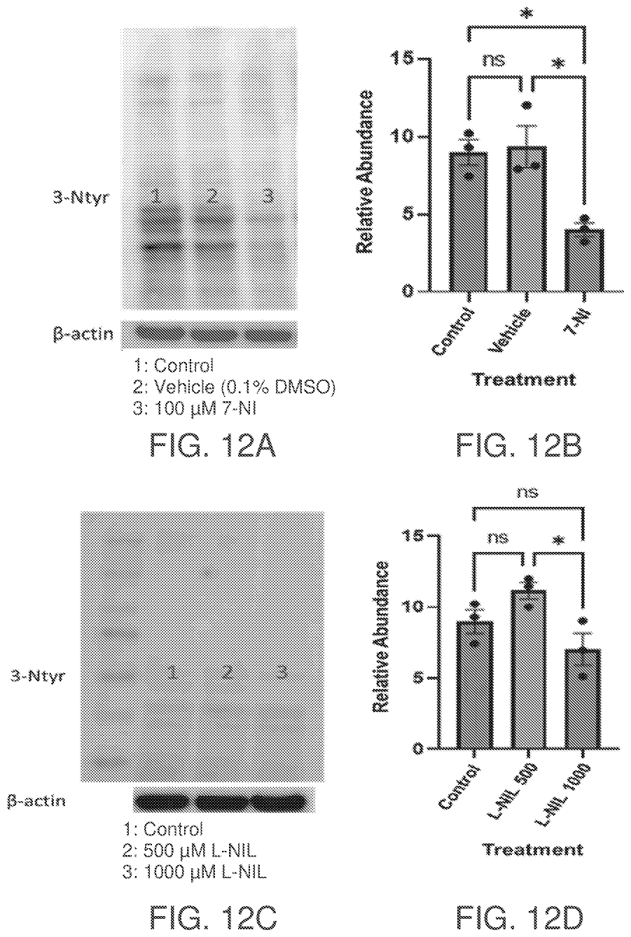

3-Ntyr is a stable NO end product and marker of increased NO production. To assess the contribution of nNOS to human glioblastoma growth, the U87-MG human glioblastoma cell line was treated with nNOS selective inhibitor (7-NI). Treatment of U87-MG cells with 7-NI significantly reduced 3-Ntyr levels compared to the control and vehicle treatments (FIGS. 12A-12B). As discussed above, aberrant NO production is theorized to be a contributor to the development and progression of glioblastoma and/or neuroblastoma—reduction in 3-Ntyr levels, which is an indicator of NO production, may indicate that the NOS inhibitor composition, e.g., 7-NI, is effective at tempering the global production of NO.

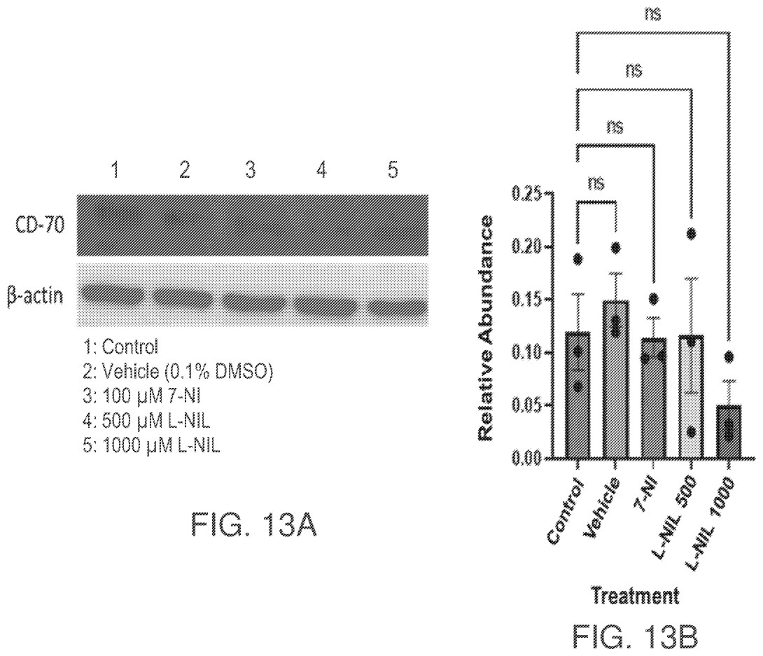

CD70 is an antigen that can be overexpressed in glioblastomas and can contribute to its aggressiveness and increase the likelihood of recurrence. For U87-MG cells following treatment with 7-NI, the expression level of CD70 slightly decreased compared to the control and vehicle treatments, though not statistically significantly (FIG. 13). The results indicate that 7-NI may confer a marginal benefit for in treating the subset of glioblastoma that is modeled by the U87-MG cell line, when considering CD70 as the predictive marker.

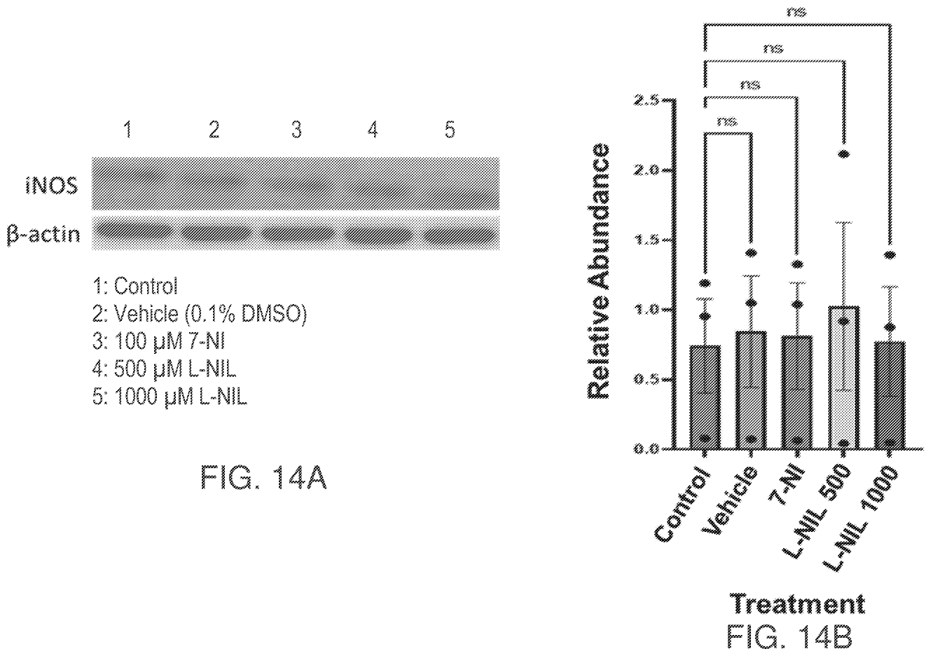

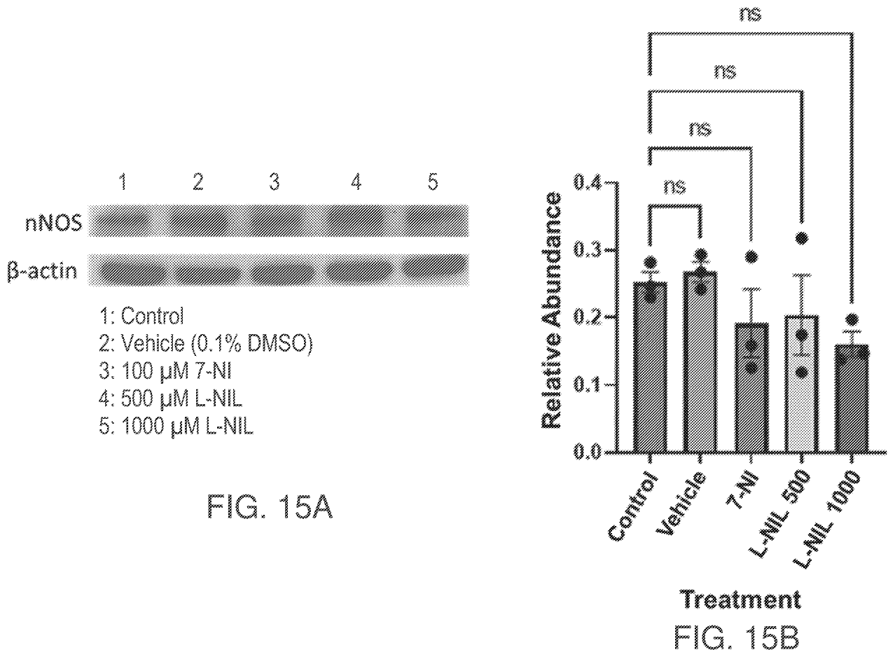

Both iNOS and nNOS can be overexpressed in glioblastomas and can contribute to its aggressiveness and increase the likelihood of recurrence. For U87-MG cells following treatment with 7-NI, the expression level of iNOS was unchanged compared to the control and vehicle treatments (FIG. 14), whereas the expression level of nNOS slightly decreased compared to the control and vehicle treatments, though not statistically significantly (FIG. 15). The results indicate that 7-NI may confer a marginal benefit for in treating the subset of glioblastoma that is modeled by the U87-MG cell line, when considering CD70 as the predictive marker.

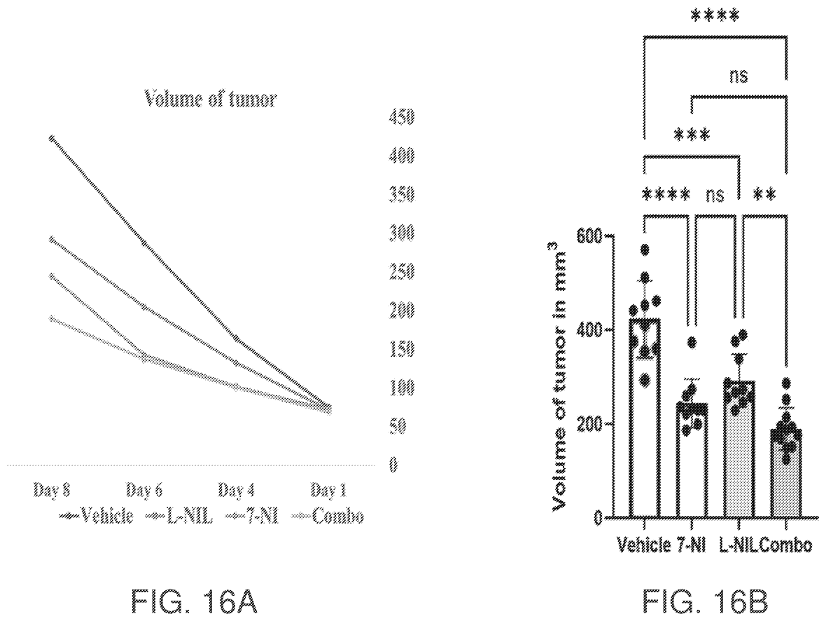

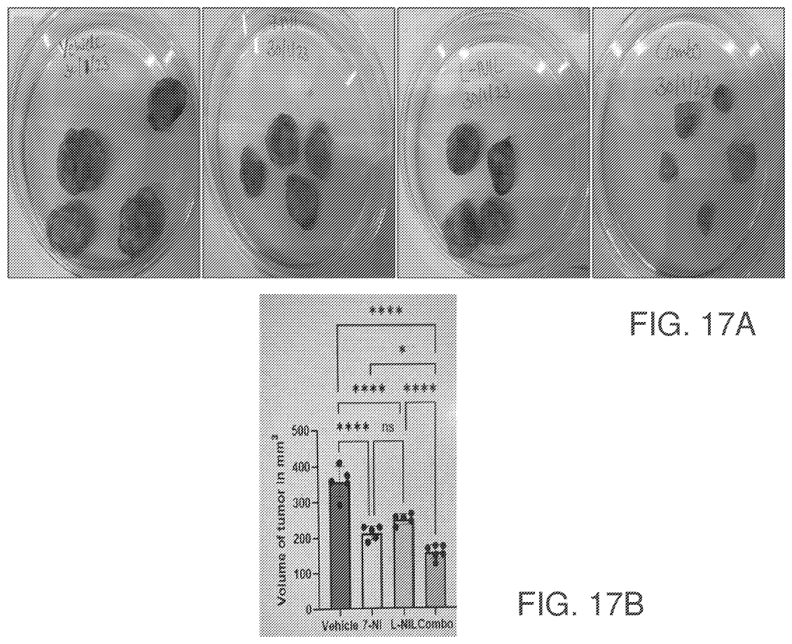

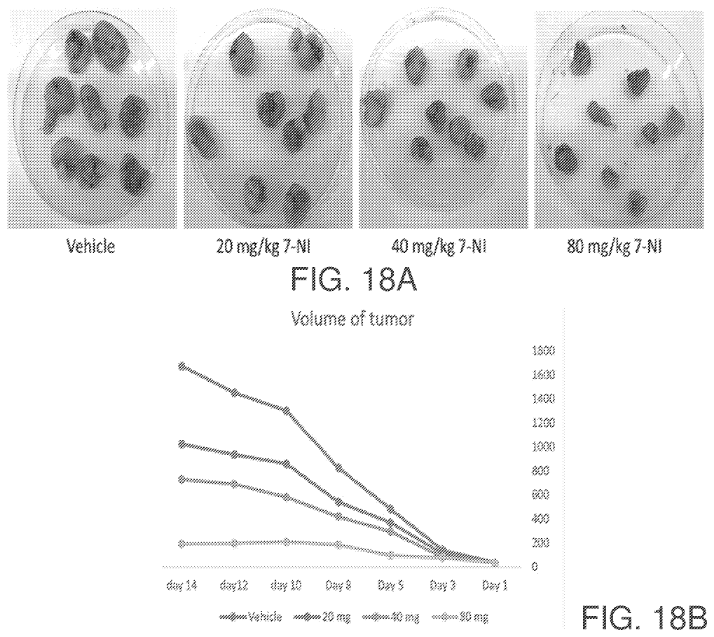

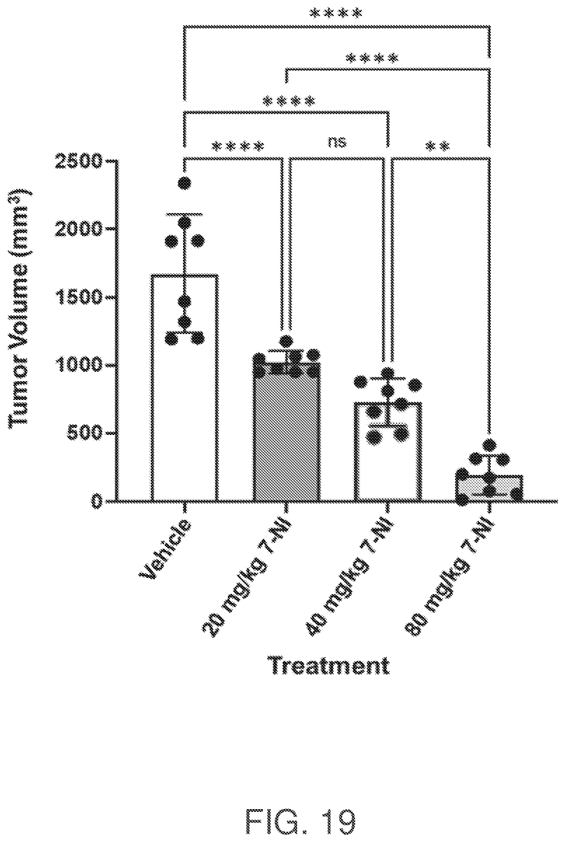

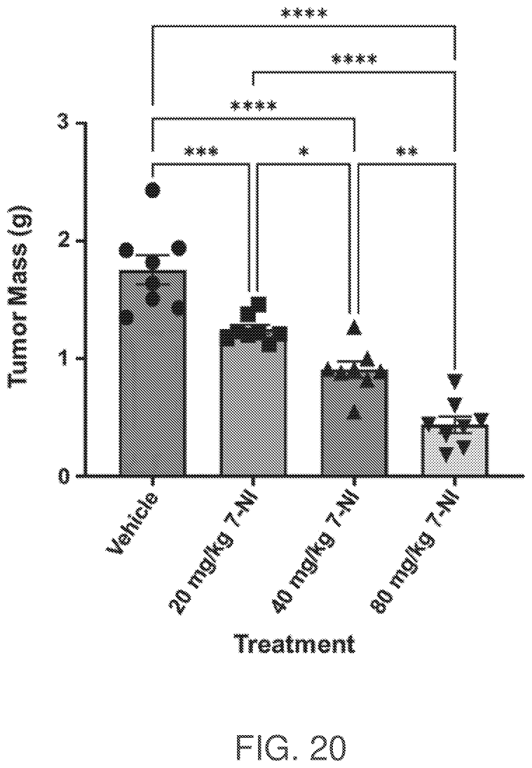

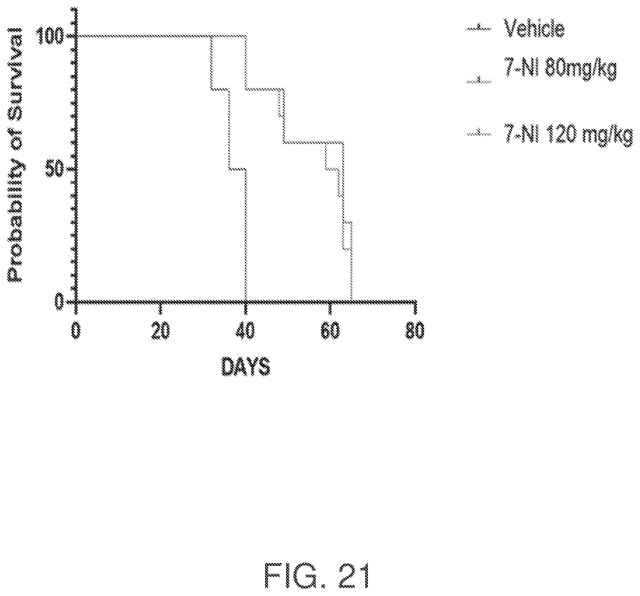

In xenografted mice, following treatment with 7-NI, mice showed a significantly smaller average tumor size compared the vehicle when administered at 80 mg/kg 7-NI (FIGS. 16-17). Notably, even at reduced dosages, i.e., as low as 20 mg/kg, 7-NI resulted in significantly smaller average tumor size (FIGS. 18-19) and smaller tumor mass (FIG. 20) compared to the vehicle, although a larger reduction of both features was observed for the highest dosage of 7-NI (FIGS. 18-20). In addition, mice treated with either 80 mg/kg or 120 mg/kg of 7-NI showed improved survival compared to vehicle-treated mice—the median survival was ~65 days in 7-NI-treated mice, compared to 35 days in vehicle-treated mice, however, there was no significant improvement was achieved by increasing the 7-NI dosage (FIG. 21).

NO takes part in the regulation of many physiological and pathological functions, which may include the development of glioblastoma and/or neuroblastoma. The results presented herein show that the inhibition of NO production in U87-MG cells (a human glioblastoma cell line), as well as the inhibition of tumor growth in U87-MG-xenografted mice by pharmacological intervention with the selective nNOS inhibitor 7-NI can effectively suppress the glioblastoma proliferation and growth, evidenced by significantly reduced levels total nitrate and nitrite content, 3-Ntyr, as well as reduced tumor growth in vivo, compared to the control.

Without being bound by any particular theory, it is believed that NO production may play a significant role in the development and progression of glioblastoma and/or neuroblastoma, among others. The effects of NO production may depend on NO concentration, exposure time, tumor type, microenvironment, and the like, however, high levels of NO (O(μM)) are suspected to cause DNA damage, and researchers have found that overexpression of NOS isoforms and excessive NO levels may play a role in the development and progression of glioblastoma and/or neuroblastoma, and may even hinder the efficacy of conventional therapies used to treat these malignancies. It is believed that inflammation can induce NO-related carcinogenesis via induction of DNA damage, suppression of DNA repair enzymes, and PTMs including SNO, because during inflammation, reactive nitrogen and oxygen species are released by activated inflammatory cells, attacking neighboring epithelial and stromal cells, altering function, and initiating carcinogenesis. The events leading to the transition to a more aggressive tumor phenotype include activation or upregulation of pro-survival/pro-expansion effector proteins, such as, for example, NF-κB, phosphoinositide-3-kinase (PI3K), AKT, p300, among others.

Collectively, the results of this study suggest that inhibition of nNOS in a human glioblastoma model (i.e., U87-MG cells), both in vitro and in vivo, may suppress the malignancy of these cells. Because nNOS appears to be an important mediator of glioblastoma survival, proliferation, and carcinogenesis, suppression of nNOS using the novel NOS inhibitor compositions disclosed herein, e.g., 7-NI, may have a vast utility in treating, ameliorating, and/or reducing the severity of glioblastoma and/or neuroblastoma, as well as others cancer where overexpression of nNOS is implicated, e.g., gastrointestinal cancers, sarcomas, ovarian cancers, cervical cancers, and the like. Moreover, because the other NOS isoforms, i.e., iNOS and eNOS, impart similar physiological effects, i.e., production of aberrant NO, the NOS inhibitor compositions disclosed herein, e.g., 7-NI, may be viable therapeutic options for pathologies wherein iNOS and/or eNOS are implicated, e.g., gastrointestinal cancers, pancreatic cancers, sarcomas, ovarian cancers, cervical cancers, lung cancers, melanomas, cancers of the head and neck, and the like.

### Example 4: 7-NI—Pancreatic Cancer

### Methods

One or more compositions individually comprising 7-NI and/or a salt of 7-NI will be prepared; the one or more individual compositions will be formulated with a dose in the range of 0.1 μg/kg to 100 mg/kg. The individual compositions will be evaluated for efficacy using an in vitro and/or in vivo model for a pancreatic cancer selected from the group consisting of an acinar cell carcinoma, an adenocarcinoma, an adenosquamous carcinoma, a colloid carcinoma, a gastrinoma, a giant cell tumor, a glucagonoma, a hepatoid carcinoma, an insulinoma, an intraductal papillary-mucinous neoplasm, a lymphoma, a mucinous cystic neoplasm, a mucinous cystic neoplasm with an invasive adenocarcinoma, a nonfunctional islet cell tumor, a pancreatic intraepithelial lesion, a pancreatic neuroendocrine tumor (PNET), a pancreatoblastoma, a PPoma, a sarcoma, a serous cystadenoma, a signet ring cell carcinoma, a solid pseudopapillary neoplasm, a somatostatinoma, a squamous cell carcinoma, an undifferentiated carcinoma, and a VIPoma, will be used instead of an in vitro and/or in vivo model for a glioblastoma or neuroblastoma.

The outcomes/endpoints discussed in Examples 1-3 will be evaluated, such outcomes/endpoints include (i) biomarker levels, such as, for example, ALDH1A3, mTOR, p-mTOR, TSC-2, AKT, pAKT, RPS6, p-RPS6, Syp, VEGF, PIGF, and/or any protein or enzyme that has an abnormal expression in the pancreatic cancer model being used; (ii) CD antigen levels, such as, for example, CD15, CD44, CD70, CD133, and/or any CD antigen that has an abnormal expression in the pancreatic cancer model being used; (iii) NO-indicator levels, such as, for example, 3-Ntyr, NADPH-diaphorase, total nitrite concentration, total nitrate concentration, total nitrite and nitrate concentration, and/or any NO indicator that has an abnormal expression in the pancreatic cancer model being used; (iv) for in vitro experiments—dose response curves, as well as the relevant outcomes/endpoints in (i)-(iii); and (v) for in vivo experiments—tumor volume reduction, overall survival, weight loss, as well as the relevant outcomes/endpoints in (i)-(iii). Additionally, outcomes/endpoints for biomarker levels, CD antigen levels, and/or NO-indicator not expressly listed above, if included, will include refer to any biomarker(s), CD antigen(s), and/or NO-indicator(s) that have an abnormal expression in the pancreatic cancer model being used.

### Example 5: 7-NI—Gastrointestinal Cancer

### Methods

Experiments will be performed in accordance with the methods disclosed is Example 4, but an in vitro and/or in vivo model for a gastrointestinal cancer selected from the group consisting of (i) a gastrointestinal cancer occurring and/or originating in the anus, such as, for example, an adenocarcinoma, a basal cell carcinoma, a carcinoma in situ, a cloacogenic carcinoma, a gastrointestinal stromal tumor, a lymphoma, a melanoma, a poorly differentiated neuroendocrine tumor, or a squamous cell carcinoma; (ii) a gastrointestinal cancer occurring and/or originating in the bile duct, such as, for example, an adenocarcinoma, a distal bile duct cancer, an extrahepatic bile duct cancer, a hilar bile duct cancer, an intrahepatic bile duct cancer, an intrahepatic cholangiocarcinoma, a lymphoma, a perihilar bile duct cancer, a sarcoma, or a squamous cell carcinoma; (iii) a gastrointestinal cancer occurring and/or originating in the colon, such as, for example, an adenocarcinoma, an adenoma, a carcinoid tumor, a familial adenomatous polyposis, a gastrointestinal stromal tumor, a leiomyosarcoma, a melanoma, a mucinous adenocarcinoma, a neuroendocrine tumor of the gastrointestinal tract, a primary colorectal lymphoma, a signet ring adenocarcinoma, or a squamous cell carcinoma; (iv) a gastrointestinal cancer occurring and/or originating in the esophagus/gastroesophageal junction, such as, for example, an adenocarcinoma, a lymphoma, a neuroendocrine tumor, a melanoma, a sarcoma, a small cell carcinoma, or a squamous cell carcinoma; (v) a gastrointestinal cancer occurring and/or originating in the gallbladder, such as, for example, an adenocarcinoma, an adenosquamous carcinoma, a carcinosarcoma, a lymphoma, a melanoma, a mucinous adenocarcinoma, a neuroendocrine tumor, a non-papillary adenocarcinoma, a papillary adenocarcinoma, a sarcoma, a small cell carcinoma, or a squamous cell carcinoma; (vi) a gastrointestinal cancer occurring and/or originating in the liver, such as, for example, an adenoma, an angiosarcoma, cholangiocarcinoma, an extrahepatic cholangiocarcinoma, fibrolamellar hepatocellular carcinoma, a focal nodular hyperplasia, a haemangioma, a hepatoblastoma, a hepatocellular carcinoma, an intrahepatic cholangiocarcinoma, or a squamous cell carcinoma; (vii) a gastrointestinal cancer occurring and/or originating in the renal system, such as, for example, an angiomyolipoma, a chromophobe carcinoma, a clear cell carcinoma, a clear cell papillary, a collecting duct carcinoma, a medullary carcinoma, a multilocular cystic carcinoma, a mucinous tubular carcinoma, a nephroblastoma, a neuroblastoma-associated renal cell carcinoma, a papillary carcinoma type I, a papillary carcinoma type II, a renal cell adenoma, a renal cell carcinoma, a renal oncocytoma, a renal sarcoma, a sarcomatoid, a spindle cell carcinoma, a transitional cell carcinoma, an unclassified carcinoma, a urothelial carcinoma, or a Wilms' tumor; (viii) a gastrointestinal cancer occurring and/or originating in the small intestine, such as, for example, an adenocarcinoma, a carcinoid tumor, a gastrinoma, a gastrointestinal stromal tumor, a leiomyosarcoma, a lymphoma, or a sarcoma; and (ix) a gastrointestinal cancer occurring and/or originating in the stomach (gastric cancer), such as, for example, an adenocarcinoma of the mucosa, an adenocarcinoma of the muscularis propria, an adenocarcinoma of the serosa, an adenocarcinoma of the submucosa, an adenocarcinoma of the subserosa, a diffuse adenocarcinoma, a gastrointestinal stromal tumor, an intestinal adenocarcinoma, an intestinal adenoma, a neuroendocrine carcinoid tumor, a neuroendocrine tumor, or a primary gastric lymphoma, will be used instead of an in vitro and/or in vivo model for a pancreatic cancer.

Additionally, outcomes/endpoints for biomarker levels, CD antigen levels, and/or NO-indicator not expressly listed above, if included, will include refer to any biomarker(s), CD antigen(s), and/or NO-indicator(s) that have an abnormal expression in the gastrointestinal cancer model being used.

### Example 6: 7-NI—Intestinal Adenoma

### Methods

Experiments will be performed in accordance with the methods disclosed is Example 4, but an in vitro and/or in vivo model for an intestinal adenoma will be used instead of an in vitro and/or in vivo model for a pancreatic cancer.

Additionally, outcomes/endpoints for biomarker levels, CD antigen levels, and/or NO-indicator not expressly listed above, if included, will include refer to any biomarker(s), CD antigen(s), and/or NO-indicator(s) that have an abnormal expression in the intestinal adenoma model being used.

### Example 7: 7-NI—Sarcoma

### Methods

Experiments will be performed in accordance with the methods disclosed is Example 4, but an in vitro and/or in vivo model for a sarcoma selected from the group consisting of an alveolar soft-part sarcoma, an angiofibroma, an angiolipoma, an angiomyelolipoma, an angiomyolipoma, an angiomyxoma, an angiosarcoma, a chondrosarcoma, a clear cell sarcoma, a dermatofibrosarcoma protuberans, a desmoid tumor, a desmoplastic small round cell tumor, an elastoma, an epithelioid sarcoma, a Ewing's sarcoma, an extraskeletal myxoid chondrosarcoma, a fibrosarcoma, a gastrointestinal stromal tumor, a giant cell tumor of a tendon sheath, a glomus tissue tumor, a granular cell tumor, a hemangioma, a hemangiopericytoma, a hibernoma, a Kaposi's sarcoma, a leiomyoma, a leiomyosarcoma, a lipoblastoma, a lipoblastomatosis, a lipoma, a lipomatosis, a liposarcoma, a lymphangioleiomyomatosis, a malignant fibrous histiocytoma, a malignant peripheral nerve sheath tumor, a myeloid sarcoma, a myoepithelial tumor of soft tissue, a myxofibrosarcoma, a myxoma, a neurofibroma, a neurofibrosarcoma, an ossifying fibromyxoid tumor, an osteosarcoma, a perivascular epithelioid cell tumor, a pigmented villonodular synovitis, a pleomorphic sarcoma, a rhabdomyosarcoma, a schwannoma, a soft tissue sarcoma, a solitary fibrous tumor, a spindle cell sarcoma, a sugar cell tumor, a synovial cell sarcoma, an undifferentiated pleomorphic sarcoma, and a uterine sarcoma, will be used instead of an in vitro and/or in vivo model for a pancreatic cancer.

Additionally, outcomes/endpoints for biomarker levels, CD antigen levels, and/or NO-indicator not expressly listed above, if included, will include refer to any biomarker(s), CD antigen(s), and/or NO-indicator(s) that have an abnormal expression in the sarcoma model being used.

### Example 8: 7-NI—Ovarian Cancer

### Methods

Experiments will be performed in accordance with the methods disclosed is Example 4, but an in vitro and/or in vivo model for an ovarian cancer selected from the group consisting of a Brenner tumor, a carcinoma, a choriocarinoma, a clear cell carcinoma, a dysgerminoma, an embryonal carcinoma, an endometrioid borderline neoplasm, an endometrioid carcinoma, an epithelial cancer, a fallopian tube cancer, a fibroma, a fibrothecoma, a granulosa cell tumor, a granulosa-theca tumor, a Krukenberg tumor, a mucinous borderline neoplasm, a mucinous carcinoma, a mucinous cystadenoma, an ovarian sarcoma, a primary peritoneal carcinoma, a serous carcinoma, a serous cystadenoma, a serous borderline neoplasm, a Sertoli cell tumor, a Sertoli-Leydig cell tumor, a small cell carcinoma, a teratoma, a thecoma, and a yolk sac tumor, and will be used instead of an in vitro and/or in vivo model for a pancreatic cancer.

Additionally, outcomes/endpoints for biomarker levels, CD antigen levels, and/or NO-indicator not expressly listed above, if included, will include refer to any biomarker(s), CD antigen(s), and/or NO-indicator(s) that have an abnormal expression in the ovarian cancer model being used.

### Example 9: 7-NI—Cervical Cancer

### Methods

Experiments will be performed in accordance with the methods disclosed is Example 4, but an in vitro and/or in vivo model for a cervical cancer selected from the group consisting of an adenocarcinoma, an adenosquamous carcinoma, a carcinoma, a clear cell carcinoma, a glassy cell carcinoma, a lymphoma, a melanoma, a mixed carcinoma, a mucinous tumor, a neuroendocrine carcinoma, a sarcoma, a small cell carcinoma, and a squamous cell carcinoma, and will be used instead of an in vitro and/or in vivo model for a pancreatic cancer.

Additionally, outcomes/endpoints for biomarker levels, CD antigen levels, and/or NO-indicator not expressly listed above, if included, will include refer to any biomarker(s), CD antigen(s), and/or NO-indicator(s) that have an abnormal expression in the cervical cancer model being used.

### Example 10: 7-NI—NO-Lung Cancer

### Methods

Experiments will be performed in accordance with the methods disclosed is Example 4, but an in vitro and/or in vivo model for a lung cancer associated with upregulation of NO signaling (“NO-lung cancer”) will be used instead of an in vitro and/or in vivo model for a pancreatic cancer.

Additionally, outcomes/endpoints for biomarker levels, CD antigen levels, and/or NO-indicator not expressly listed above, if included, will include refer to any biomarker(s), CD antigen(s), and/or NO-indicator(s) that have an abnormal expression in the NO-lung cancer model being used.

### Example 11: 7-NI—NSC-Lung Cancer

### Methods

Experiments will be performed in accordance with the methods disclosed is Example 4, but an in vitro and/or in vivo model for a non-small cell lung cancer (“NSC-lung cancer”) will be used instead of an in vitro and/or in vivo model for a pancreatic cancer.

Additionally, outcomes/endpoints for biomarker levels, CD antigen levels, and/or NO-indicator not expressly listed above, if included, will include refer to any biomarker(s), CD antigen(s), and/or NO-indicator(s) that have an abnormal expression in the NSC-lung cancer model being used.

### Example 12: 7-NI—m-HNSCC

### Methods

Experiments will be performed in accordance with the methods disclosed is Example 4, but an in vitro and/or in vivo model for a metastatic squamous cell carcinoma of the head and neck (“m-HNSCC”) will be used instead of an in vitro and/or in vivo model for a pancreatic cancer.

Additionally, outcomes/endpoints for biomarker levels, CD antigen levels, and/or NO-indicator not expressly listed above, if included, will include refer to any biomarker(s), CD antigen(s), and/or NO-indicator(s) that have an abnormal expression in the m-HNSCC model being used.

### Example 13: 7-NI—s-SCC

### Methods

Experiments will be performed in accordance with the methods disclosed is Example 4, but an in vitro and/or in vivo model for a squamous cell carcinoma of the skin (“s-SCC”) will be used instead of an in vitro and/or in vivo model for a pancreatic cancer.

Additionally, outcomes/endpoints for biomarker levels, CD antigen levels, and/or NO-indicator not expressly listed above, if included, will include refer to any biomarker(s), CD antigen(s), and/or NO-indicator(s) that have an abnormal expression in the s-SCC model being used.

### Example 14: 3-Br-7-NI—Glioblastoma

### Methods

Experiments will be performed in accordance with the methods disclosed in Example 4, but the one or more compositions will be prepared to comprise 3-Br-7-NI and/or a salt of 3-Br-7-NI instead of 7-NI and/or a salt of 7-NI. Additionally, the experiments will use an in vitro and/or in vivo model for glioblastoma, such as that disclosed in Example 3, instead of an in vitro and/or in vivo model for a pancreatic cancer.

Additionally, outcomes/endpoints for biomarker levels, CD antigen levels, and/or NO-indicator not expressly listed above, if included, will include refer to any biomarker(s), CD antigen(s), and/or NO-indicator(s) that have an abnormal expression in the glioblastoma model being used.

### Example 15: 3-Br-7-NI—Neuroblastoma

### Methods

Experiments will be performed in accordance with the methods disclosed in Example 4, but the one or more compositions will be prepared to comprise 3-Br-7-NI and/or a salt of 3-Br-7-NI instead of 7-NI and/or a salt of 7-NI. Additionally, the experiments will use an in vitro and/or in vivo model for neuroblastoma, such as that disclosed in Example 1, instead of an in vitro and/or in vivo model for a pancreatic cancer.

Additionally, outcomes/endpoints for biomarker levels, CD antigen levels, and/or NO-indicator not expressly listed above, if included, will include refer to any biomarker(s), CD antigen(s), and/or NO-indicator(s) that have an abnormal expression in the neuroblastoma model being used.

### Example 16: 3-Br-7-NI—Pancreatic Cancer

### Methods

Experiments will be performed in accordance with the methods disclosed in Example 4, but the one or more compositions will be prepared to comprise 3-Br-7-NI and/or a salt of 3-Br-7-NI instead of 7-NI and/or a salt of 7-NI.

### Example 17: 3-Br-7-NI—Gastrointestinal Cancer

### Methods

Experiments will be performed in accordance with the methods disclosed in Example 5, but the one or more compositions will be prepared to comprise 3-Br-7-NI and/or a salt of 3-Br-7-NI instead of 7-NI and/or a salt of 7-NI.

### Example 18: 3-Br-7-NI—Intestinal Adenoma

### Methods

Experiments will be performed in accordance with the methods disclosed in Example 6, but the one or more compositions will be prepared to comprise 3-Br-7-NI and/or a salt of 3-Br-7-NI instead of 7-NI and/or a salt of 7-NI.

### Example 19: 3-Br-7-NI—Sarcoma

### Methods

Experiments will be performed in accordance with the methods disclosed in Example 7, but the one or more compositions will be prepared to comprise 3-Br-7-NI and/or a salt of 3-Br-7-NI instead of 7-NI and/or a salt of 7-NI.

### Example 20: 3-Br-7-NI—Ovarian Cancer

### Methods

Experiments will be performed in accordance with the methods disclosed in Example 8, but the one or more compositions will be prepared to comprise 3-Br-7-NI and/or a salt of 3-Br-7-NI instead of 7-NI and/or a salt of 7-NI.

### Example 21: 3-Br-7-NI—Cervical Cancer

### Methods

Experiments will be performed in accordance with the methods disclosed in Example 9, but the one or more compositions will be prepared to comprise 3-Br-7-NI and/or a salt of 3-Br-7-NI instead of 7-NI and/or a salt of 7-NI.

### Example 22: 3-Br-7-NI—NO-Lung Cancer

### Methods

Experiments will be performed in accordance with the methods disclosed in Example 10, but the one or more compositions will be prepared to comprise 3-Br-7-NI and/or a salt of 3-Br-7-NI instead of 7-NI and/or a salt of 7-NI.

### Example 23: 3-Br-7-NI—NSC-Lung Cancer

### Methods

Experiments will be performed in accordance with the methods disclosed in Example 11, but the one or more compositions will be prepared to comprise 3-Br-7-NI and/or a salt of 3-Br-7-NI instead of 7-NI and/or a salt of 7-NI.

### Example 24: 3-Br-7-NI—m-HNSCC

### Methods

Experiments will be performed in accordance with the methods disclosed in Example 12, but the one or more compositions will be prepared to comprise 3-Br-7-NI and/or a salt of 3-Br-7-NI instead of 7-NI and/or a salt of 7-NI.

### Example 25: 3-Br-7-NI—s-SCC

### Methods

Experiments will be performed in accordance with the methods disclosed in Example 13, but the one or more compositions will be prepared to comprise 3-Br-7-NI and/or a salt of 3-Br-7-NI instead of 7-NI and/or a salt of 7-NI.

### Example 26: L-NPA—Glioblastoma

### Methods

Experiments will be performed in accordance with the methods disclosed in Example 14, but the one or more compositions will be prepared to comprise L-NPA and/or L-NPA hydrochloride instead of 3-Br-7-NI and/or a salt of 3-Br-7-NI.

### Example 27: L-NPA—Neuroblastoma

### Methods

Experiments will be performed in accordance with the methods disclosed in Example 15, but the one or more compositions will be prepared to comprise L-NPA and/or L-NPA hydrochloride instead of 3-Br-7-NI and/or a salt of 3-Br-7-NI.

### Example 28: L-NPA—Pancreatic Cancer

### Methods

Experiments will be performed in accordance with the methods disclosed in Example 4, but the one or more compositions will be prepared to comprise L-NPA and/or L-NPA hydrochloride instead of 7-NI and/or a salt of 7-NI.

### Example 29: L-NPA—Gastrointestinal Cancer

### Methods

Experiments will be performed in accordance with the methods disclosed in Example 5, but the one or more compositions will be prepared to comprise L-NPA and/or L-NPA hydrochloride instead of 7-NI and/or a salt of 7-NI.

### Example 30: L-NPA—Intestinal Adenoma

### Methods

Experiments will be performed in accordance with the methods disclosed in Example 6, but the one or more compositions will be prepared to comprise L-NPA and/or L-NPA hydrochloride instead of 7-NI and/or a salt of 7-NI.

### Example 31: L-NPA—Sarcoma

### Methods

Experiments will be performed in accordance with the methods disclosed in Example 7, but the one or more compositions will be prepared to comprise L-NPA and/or L-NPA hydrochloride instead of 7-NI and/or a salt of 7-NI.

### Example 32: L-NPA—Ovarian Cancer

### Methods

Experiments will be performed in accordance with the methods disclosed in Example 8, but the one or more compositions will be prepared to comprise L-NPA and/or L-NPA hydrochloride instead of 7-NI and/or a salt of 7-NI.

### Example 33: L-NPA—Cervical Cancer

### Methods

Experiments will be performed in accordance with the methods disclosed in Example 9, but the one or more compositions will be prepared to comprise L-NPA and/or L-NPA hydrochloride instead of 7-NI and/or a salt of 7-NI.

### Example 34: L-NPA—NO-Lung Cancer

### Methods

Experiments will be performed in accordance with the methods disclosed in Example 10, but the one or more compositions will be prepared to comprise L-NPA and/or L-NPA hydrochloride instead of 7-NI and/or a salt of 7-NI.

### Example 35: L-NPA—NSC-Lung Cancer

### Methods

Experiments will be performed in accordance with the methods disclosed in Example 11, but the one or more compositions will be prepared to comprise L-NPA and/or L-NPA hydrochloride instead of 7-NI and/or a salt of 7-NI.

### Example 36: L-NPA—m-HNSCC

### Methods

Experiments will be performed in accordance with the methods disclosed in Example 12, but the one or more compositions will be prepared to comprise L-NPA and/or L-NPA hydrochloride instead of 7-NI and/or a salt of 7-NI.

### Example 37: L-NPA—s-SCC

### Methods

Experiments will be performed in accordance with the methods disclosed in Example 13, but the one or more compositions will be prepared to comprise L-NPA and/or L-NPA hydrochloride instead of 7-NI and/or a salt of 7-NI.

### Example 38: L-NIO—Glioblastoma

### Methods

Experiments will be performed in accordance with the methods disclosed in Example 14, but the one or more compositions will be prepared to comprise L-NIO and/or L-NIO dihydrochloride instead of 3-Br-7-NI and/or a salt of 3-Br-7-NI.

### Example 39: L-NIO—Neuroblastoma

### Methods

Experiments will be performed in accordance with the methods disclosed in Example 15, but the one or more compositions will be prepared to comprise L-NIO and/or L-NIO dihydrochloride instead of 3-Br-7-NI and/or a salt of 3-Br-7-NI.

### Example 40: L-NIO—Pancreatic Cancer

### Methods

Experiments will be performed in accordance with the methods disclosed in Example 4, but the one or more compositions will be prepared to comprise L-NIO and/or L-NIO dihydrochloride instead of 7-NI and/or a salt of 7-NI.

### Example 41: L-NIO—Gastrointestinal Cancer

### Methods

Experiments will be performed in accordance with the methods disclosed in Example 5, but the one or more compositions will be prepared to comprise L-NIO and/or L-NIO dihydrochloride instead of 7-NI and/or a salt of 7-NI.

### Example 42: L-NIO—Intestinal Adenoma

### Methods

Experiments will be performed in accordance with the methods disclosed in Example 6, but the one or more compositions will be prepared to comprise L-NIO and/or L-NIO dihydrochloride instead of 7-NI and/or a salt of 7-NI.

### Example 43: L-NIO—Sarcoma

### Methods

Experiments will be performed in accordance with the methods disclosed in Example 7, but the one or more compositions will be prepared to comprise L-NIO and/or L-NIO dihydrochloride instead of 7-NI and/or a salt of 7-NI.

### Example 44: L-NIO—Ovarian Cancer

### Methods

Experiments will be performed in accordance with the methods disclosed in Example 8, but the one or more compositions will be prepared to comprise L-NIO and/or L-NIO dihydrochloride instead of 7-NI and/or a salt of 7-NI.

### Example 45: L-NIO—Cervical Cancer

### Methods

Experiments will be performed in accordance with the methods disclosed in Example 9, but the one or more compositions will be prepared to comprise L-NIO and/or L-NIO dihydrochloride instead of 7-NI and/or a salt of 7-NI.

### Example 46: L-NIO—NO-Lung Cancer

### Methods

Experiments will be performed in accordance with the methods disclosed in Example 10, but the one or more compositions will be prepared to comprise L-NIO and/or L-NIO dihydrochloride instead of 7-NI and/or a salt of 7-NI.

### Example 47: L-NIO—NSC-Lung Cancer

### Methods

Experiments will be performed in accordance with the methods disclosed in Example 11, but the one or more compositions will be prepared to comprise L-NIO and/or L-NIO dihydrochloride instead of 7-NI and/or a salt of 7-NI.

### Example 48: L-NIO—m-HNSCC

### Methods

Experiments will be performed in accordance with the methods disclosed in Example 12, but the one or more compositions will be prepared to comprise L-NIO and/or L-NIO dihydrochloride instead of 7-NI and/or a salt of 7-NI.

### Example 49: L-NIO—s-SCC

### Methods

Experiments will be performed in accordance with the methods disclosed in Example 13, but the one or more compositions will be prepared to comprise L-NIO and/or L-NIO dihydrochloride instead of 7-NI and/or a salt of 7-NI.

### Example 50: ARL-17477—Glioblastoma

### Methods

Experiments will be performed in accordance with the methods disclosed in Example 14, but the one or more compositions will be prepared to comprise ARL-17477 and/or ARL-17477 dihydrochloride instead of 3-Br-7-NI and/or a salt of 3-Br-7-NI.

### Example 51: ARL-17477—Neuroblastoma

### Methods

Experiments will be performed in accordance with the methods disclosed in Example 15, but the one or more compositions will be prepared to comprise ARL-17477 and/or ARL-17477 dihydrochloride instead of 3-Br-7-NI and/or a salt of 3-Br-7-NI.

### Example 52: ARL-17477—Pancreatic Cancer

### Methods

Experiments will be performed in accordance with the methods disclosed in Example 4, but the one or more compositions will be prepared to comprise ARL-17477 and/or ARL-17477 dihydrochloride instead of 7-NI and/or a salt of 7-NI.

### Example 53: ARL-17477—Gastrointestinal Cancer

### Methods

Experiments will be performed in accordance with the methods disclosed in Example 5, but the one or more compositions will be prepared to comprise ARL-17477 and/or ARL-17477 dihydrochloride instead of 7-NI and/or a salt of 7-NI.

### Example 54: ARL-17477—Intestinal Adenoma

### Methods

Experiments will be performed in accordance with the methods disclosed in Example 6, but the one or more compositions will be prepared to comprise ARL-17477 and/or ARL-17477 dihydrochloride instead of 7-NI and/or a salt of 7-NI.

### Example 55: ARL-17477—Sarcoma

### Methods

Experiments will be performed in accordance with the methods disclosed in Example 7, but the one or more compositions will be prepared to comprise ARL-17477 and/or ARL-17477 dihydrochloride instead of 7-NI and/or a salt of 7-NI.

### Example 56: ARL-17477—Ovarian Cancer

### Methods

Experiments will be performed in accordance with the methods disclosed in Example 8, but the one or more compositions will be prepared to comprise ARL-17477 and/or ARL-17477 dihydrochloride instead of 7-NI and/or a salt of 7-NI.

### Example 57: ARL-17477—Cervical Cancer

### Methods

Experiments will be performed in accordance with the methods disclosed in Example 9, but the one or more compositions will be prepared to comprise ARL-17477 and/or ARL-17477 dihydrochloride instead of 7-NI and/or a salt of 7-NI.

### Example 58: ARL-17477—NO-Lung Cancer

### Methods

Experiments will be performed in accordance with the methods disclosed in Example 10, but the one or more compositions will be prepared to comprise ARL-17477 and/or ARL-17477 dihydrochloride instead of 7-NI and/or a salt of 7-NI.

### Example 59: ARL-17477—NSC-Lung Cancer

### Methods

Experiments will be performed in accordance with the methods disclosed in Example 11, but the one or more compositions will be prepared to comprise ARL-17477 and/or ARL-17477 dihydrochloride instead of 7-NI and/or a salt of 7-NI.

### Example 60: ARL-17477—m-HNSCC

### Methods

Experiments will be performed in accordance with the methods disclosed in Example 12, but the one or more compositions will be prepared to comprise ARL-17477 and/or ARL-17477 dihydrochloride instead of 7-NI and/or a salt of 7-NI.

### Example 61: ARL-17477—s-SCC

### Methods

Experiments will be performed in accordance with the methods disclosed in Example 13, but the one or more compositions will be prepared to comprise ARL-17477 and/or ARL-17477 dihydrochloride instead of 7-NI and/or a salt of 7-NI.

### Example 62: L-NNA—Glioblastoma

### Methods

Experiments will be performed in accordance with the methods disclosed in Example 14, but the one or more compositions will be prepared to comprise L-NNA and/or a salt thereof instead of 3-Br-7-NI and/or a salt of 3-Br-7-NI.

### Example 63: L-NNA—Neuroblastoma

### Methods

Experiments will be performed in accordance with the methods disclosed in Example 15, but the one or more compositions will be prepared to comprise L-NNA and/or a salt thereof instead of 3-Br-7-NI and/or a salt of 3-Br-7-NI.

### Example 64: L-NNA—Pancreatic Cancer

### Methods

Experiments will be performed in accordance with the methods disclosed in Example 4, but the one or more compositions will be prepared to comprise L-NNA and/or a salt thereof instead of 7-NI and/or a salt of 7-NI.

### Example 65: L-NNA—Gastrointestinal Cancer

### Methods

Experiments will be performed in accordance with the methods disclosed in Example 5, but the one or more compositions will be prepared to comprise L-NNA and/or a salt thereof instead of 7-NI and/or a salt of 7-NI.

### Example 66: L-NNA—Intestinal Adenoma

### Methods

Experiments will be performed in accordance with the methods disclosed in Example 6, but the one or more compositions will be prepared to comprise L-NNA and/or a salt thereof instead of 7-NI and/or a salt of 7-NI.

### Example 67: L-NNA—Sarcoma

### Methods

Experiments will be performed in accordance with the methods disclosed in Example 7, but the one or more compositions will be prepared to comprise L-NNA and/or a salt thereof instead of 7-NI and/or a salt of 7-NI.

### Example 68: L-NNA—Ovarian Cancer

### Methods

Experiments will be performed in accordance with the methods disclosed in Example 8, but the one or more compositions will be prepared to comprise L-NNA and/or a salt thereof instead of 7-NI and/or a salt of 7-NI.

### Example 69: L-NNA—Cervical Cancer

### Methods

Experiments will be performed in accordance with the methods disclosed in Example 9, but the one or more compositions will be prepared to comprise L-NNA and/or a salt thereof instead of 7-NI and/or a salt of 7-NI.

### Example 70: L-NNA—NO-Lung Cancer

### Methods

Experiments will be performed in accordance with the methods disclosed in Example 10, but the one or more compositions will be prepared to comprise L-NNA and/or a salt thereof instead of 7-NI and/or a salt of 7-NI.

### Example 71: L-NNA—NSC-Lung Cancer

### Methods

Experiments will be performed in accordance with the methods disclosed in Example 11, but the one or more compositions will be prepared to comprise L-NNA and/or a salt thereof instead of 7-NI and/or a salt of 7-NI.

### Example 72: L-NNA—m-HNSCC

### Methods

Experiments will be performed in accordance with the methods disclosed in Example 12, but the one or more compositions will be prepared to comprise L-NNA and/or a salt thereof instead of 7-NI and/or a salt of 7-NI.

### Example 73: L-NNA—s-SCC

### Methods

Experiments will be performed in accordance with the methods disclosed in Example 13, but the one or more compositions will be prepared to comprise L-NNA and/or a salt thereof instead of 7-NI and/or a salt of 7-NI.

### Example 74: L-NAME—Glioblastoma

### Methods

Experiments will be performed in accordance with the methods disclosed in Example 14, but the one or more compositions will be prepared to comprise L-NAME and/or L-NAME hydrochloride instead of 3-Br-7-NI and/or a salt of 3-Br-7-NI.

### Example 75: L-NAME—Neuroblastoma

### Methods

Experiments will be performed in accordance with the methods disclosed in Example 15, but the one or more compositions will be prepared to comprise L-NAME and/or L-NAME hydrochloride instead of 3-Br-7-NI and/or a salt of 3-Br-7-NI.

### Example 76: L-NAME—Pancreatic Cancer

### Methods

Experiments will be performed in accordance with the methods disclosed in Example 4, but the one or more compositions will be prepared to comprise L-NAME and/or L-NAME hydrochloride instead of 7-NI and/or a salt of 7-NI.

### Example 77: L-NAME—Gastrointestinal Cancer

### Methods

Experiments will be performed in accordance with the methods disclosed in Example 5, but the one or more compositions will be prepared to comprise L-NAME and/or L-NAME hydrochloride instead of 7-NI and/or a salt of 7-NI.

### Example 78: L-NAME—Intestinal Adenoma

### Methods

Experiments will be performed in accordance with the methods disclosed in Example 6, but the one or more compositions will be prepared to comprise L-NAME and/or L-NAME hydrochloride instead of 7-NI and/or a salt of 7-NI.

### Example 79: L-NAME—Sarcoma

### Methods

Experiments will be performed in accordance with the methods disclosed in Example 7, but the one or more compositions will be prepared to comprise L-NAME and/or L-NAME hydrochloride instead of 7-NI and/or a salt of 7-NI.

### Example 80: L-NAME—Ovarian Cancer

### Methods

Experiments will be performed in accordance with the methods disclosed in Example 8, but the one or more compositions will be prepared to comprise L-NAME and/or L-NAME hydrochloride instead of 7-NI and/or a salt of 7-NI.

### Example 81: L-NAME—Cervical Cancer

### Methods

Experiments will be performed in accordance with the methods disclosed in Example 9, but the one or more compositions will be prepared to comprise L-NAME and/or L-NAME hydrochloride instead of 7-NI and/or a salt of 7-NI.

### Example 82: L-NAME—NO-Lung Cancer

### Methods

Experiments will be performed in accordance with the methods disclosed in Example 10, but the one or more compositions will be prepared to comprise L-NAME and/or L-NAME hydrochloride instead of 7-NI and/or a salt of 7-NI.

### Example 83: L-NAME—NSC-Lung Cancer

### Methods

Experiments will be performed in accordance with the methods disclosed in Example 11, but the one or more compositions will be prepared to comprise L-NAME and/or L-NAME hydrochloride instead of 7-NI and/or a salt of 7-NI.

### Example 84: L-NAME—m-HNSCC

### Methods

Experiments will be performed in accordance with the methods disclosed in Example 12, but the one or more compositions will be prepared to comprise L-NAME and/or L-NAME hydrochloride instead of 7-NI and/or a salt of 7-NI.

### Example 85: L-NAME—s-SCC

### Methods

Experiments will be performed in accordance with the methods disclosed in Example 13, but the one or more compositions will be prepared to comprise L-NAME and/or L-NAME hydrochloride instead of 7-NI and/or a salt of 7-NI.

### Example 86: L-NMMA—Glioblastoma

### Methods

Experiments will be performed in accordance with the methods disclosed in Example 14, but the one or more compositions will be prepared to comprise L-NMMA and/or L-NMMA hydrochloride instead of 3-Br-7-NI and/or a salt of 3-Br-7-NI.

### Example 87: L-NMMA—Neuroblastoma

### Methods

Experiments will be performed in accordance with the methods disclosed in Example 15, but the one or more compositions will be prepared to comprise L-NMMA and/or L-NMMA hydrochloride instead of 3-Br-7-NI and/or a salt of 3-Br-7-NI.

### Example 88: L-NMMA—Pancreatic Cancer

### Methods

Experiments will be performed in accordance with the methods disclosed in Example 4, but the one or more compositions will be prepared to comprise L-NMMA and/or L-NMMA hydrochloride instead of 7-NI and/or a salt of 7-NI.

### Example 89: L-NMMA—Gastrointestinal Cancer

### Methods

Experiments will be performed in accordance with the methods disclosed in Example 5, but the one or more compositions will be prepared to comprise L-NMMA and/or L-NMMA hydrochloride instead of 7-NI and/or a salt of 7-NI.

### Example 90: L-NMMA—Intestinal Adenoma

### Methods

Experiments will be performed in accordance with the methods disclosed in Example 6, but the one or more compositions will be prepared to comprise L-NMMA and/or L-NMMA hydrochloride instead of 7-NI and/or a salt of 7-NI.

### Example 91: L-NMMA—Sarcoma

### Methods

Experiments will be performed in accordance with the methods disclosed in Example 7, but the one or more compositions will be prepared to comprise L-NMMA and/or L-NMMA hydrochloride instead of 7-NI and/or a salt of 7-NI.

### Example 92: L-NMMA—Ovarian Cancer

### Methods

Experiments will be performed in accordance with the methods disclosed in Example 8, but the one or more compositions will be prepared to comprise L-NMMA and/or L-NMMA hydrochloride instead of 7-NI and/or a salt of 7-NI.

### Example 93: L-NMMA—Cervical Cancer

### Methods

Experiments will be performed in accordance with the methods disclosed in Example 9, but the one or more compositions will be prepared to comprise L-NMMA and/or L-NMMA hydrochloride instead of 7-NI and/or a salt of 7-NI.

### Example 94: L-NMMA—NO-Lung Cancer

### Methods

Experiments will be performed in accordance with the methods disclosed in Example 10, but the one or more compositions will be prepared to comprise L-NMMA and/or L-NMMA hydrochloride instead of 7-NI and/or a salt of 7-NI.

### Example 95: L-NMMA—NSC-Lung Cancer

### Methods

Experiments will be performed in accordance with the methods disclosed in Example 11, but the one or more compositions will be prepared to comprise L-NMMA and/or L-NMMA hydrochloride instead of 7-NI and/or a salt of 7-NI.

### Example 96: L-NMMA—m-HNSCC

### Methods

Experiments will be performed in accordance with the methods disclosed in Example 12, but the one or more compositions will be prepared to comprise L-NMMA and/or L-NMMA hydrochloride instead of 7-NI and/or a salt of 7-NI.

### Example 97: L-NMMA—s-SCC

### Methods

Experiments will be performed in accordance with the methods disclosed in Example 13, but the one or more compositions will be prepared to comprise L-NMMA and/or L-NMMA hydrochloride instead of 7-NI and/or a salt of 7-NI.

### Example 98: GW-274150—Glioblastoma

### Methods

Experiments will be performed in accordance with the methods disclosed in Example 14, but the one or more compositions will be prepared to comprise GW-274150 and/or GW-274150 phosphate instead of 3-Br-7-NI and/or a salt of 3-Br-7-NI.

### Example 99: GW-274150—Neuroblastoma

### Methods

Experiments will be performed in accordance with the methods disclosed in Example 15, but the one or more compositions will be prepared to comprise GW-274150 and/or GW-274150 phosphate instead of 3-Br-7-NI and/or a salt of 3-Br-7-NI.

### Example 100: GW-274150—Pancreatic Cancer

### Methods

Experiments will be performed in accordance with the methods disclosed in Example 4, but the one or more compositions will be prepared to comprise GW-274150 and/or GW-274150 phosphate instead of 7-NI and/or a salt of 7-NI.

### Example 101: GW-274150—Gastrointestinal Cancer

### Methods

Experiments will be performed in accordance with the methods disclosed in Example 5, but the one or more compositions will be prepared to comprise GW-274150 and/or GW-274150 phosphate instead of 7-NI and/or a salt of 7-NI.

### Example 102: GW-274150—Intestinal Adenoma

### Methods

Experiments will be performed in accordance with the methods disclosed in Example 6, but the one or more compositions will be prepared to comprise GW-274150 and/or GW-274150 phosphate instead of 7-NI and/or a salt of 7-NI.

### Example 103: GW-274150—Sarcoma

### Methods

Experiments will be performed in accordance with the methods disclosed in Example 7, but the one or more compositions will be prepared to comprise GW-274150 and/or GW-274150 phosphate instead of 7-NI and/or a salt of 7-NI.

### Example 104: GW-274150—Ovarian Cancer

### Methods

Experiments will be performed in accordance with the methods disclosed in Example 8, but the one or more compositions will be prepared to comprise GW-274150 and/or GW-274150 phosphate instead of 7-NI and/or a salt of 7-NI.

### Example 105: GW-274150—Cervical Cancer

### Methods

Experiments will be performed in accordance with the methods disclosed in Example 9, but the one or more compositions will be prepared to comprise GW-274150 and/or GW-274150 phosphate instead of 7-NI and/or a salt of 7-NI.

### Example 106: GW-274150—NO-Lung Cancer

### Methods

Experiments will be performed in accordance with the methods disclosed in Example 10, but the one or more compositions will be prepared to comprise GW-274150 and/or GW-274150 phosphate instead of 7-NI and/or a salt of 7-NI.

### Example 107: GW-274150—NSC-Lung Cancer

### Methods

Experiments will be performed in accordance with the methods disclosed in Example 11, but the one or more compositions will be prepared to comprise GW-274150 and/or GW-274150 phosphate instead of 7-NI and/or a salt of 7-NI.

### Example 108: GW-274150—m-HNSCC

### Methods

Experiments will be performed in accordance with the methods disclosed in Example 12, but the one or more compositions will be prepared to comprise GW-274150 and/or GW-274150 phosphate instead of 7-NI and/or a salt of 7-NI.

### Example 109: GW-274150—s-SCC

### Methods

Experiments will be performed in accordance with the methods disclosed in Example 13, but the one or more compositions will be prepared to comprise GW-274150 and/or GW-274150 phosphate instead of 7-NI and/or a salt of 7-NI.

### Example 110: L-NIL—Glioblastoma

### Methods

Experiments will be performed in accordance with the methods disclosed in Example 14, but the one or more compositions will be prepared to comprise L-NIL and/or L-NIL hydrochloride instead of 3-Br-7-NI and/or a salt of 3-Br-7-NI.

In a particular example, in vitro and in vivo experiments were performed using U87-MG cells and U87-MG-xenografted mice, respectively, as a glioblastoma model. The same methods that were utilized in Example 3 were utilized in Example 110. The difference in Example 110 compared to Example 3 is that L-NIL was administered in vitro and in vivo instead of 7-NI. The in vitro dose response curve for L-NIL is presented in FIG. 7.

### Results

In the colony assay, L-NIL treatment resulted in a decreased colony growth compared to the control for both doses of L-NIL evaluated in two separate experiments (FIGS. 8C-8D and 9). In one experiment the lower dose of L-NIL significantly reduced colony growth compared to the control (FIG. 9), whereas in a second experiment, the lower dose of L-NIL reduced colony growth compared to the control, however the reduction was not statistically significant (FIGS. 8C-8D). At the higher dose of L-NIL, however, both experiments showed a significant decrease in colony growth compared the control for both doses of L-NIL evaluated (FIGS. 8C-8D and 9). Because L-NIL is an iNOS-specific inhibitor, these results indicate that the observed decrease in the number of colonies following treatment with L-NIL may occur via suppression of iNOS in U87-MG cells.

In the Griess assay, L-NIL treatment resulted in a reduction of nitrite and nitrate content in U87-MG cells compared to the control for both doses of L-NIL evaluated in two separate experiments (FIGS. 10B and 11). In one experiment the lower dose of L-NIL significantly reduced nitrite and nitrate content compared to the control (FIG. 11), whereas in a second experiment, the lower dose of L-NIL reduced colony growth compared to the control, however the reduction was not statistically significant (FIG. 10B). Similar results were observed at the higher dose of L-NIL—in one experiment the higher dose of L-NIL significantly reduced nitrite and nitrate content compared to the control (FIG. 10B), whereas in a second experiment, the higher dose of L-NIL reduced colony growth compared to the control, however the reduction was not statistically significant (FIG. 11). Because L-NIL is an iNOS-specific inhibitor, these results indicate that the observed decrease in nitrite and nitrate content may occur because of global iNOS activity suppression.

3-Ntyr is a stable NO end product and marker of increased NO production. To assess the contribution of iNOS to human glioblastoma growth, the U87-MG human glioblastoma cell line was treated with an iNOS selective inhibitor (L-NIL). Treatment of U87-MG cells with the lower dose of L-NIL slightly increased 3-Ntyr levels, whereas the higher dose of L-NIL reduced 3-Ntyr levels compared to the control, though neither result was significantly significant (FIGS. 12C-12D). As discussed above, aberrant NO production is theorized to be a contributor to the development and progression of glioblastoma and/or neuroblastoma—reduction in 3-Ntyr levels, as seen for the higher dose L-NIL treatment, and which is an indicator of NO production, may indicate that the NOS inhibitor composition, e.g., L-NIL, may be effective at tempering the global production of NO.

CD70 is an antigen that can be overexpressed in glioblastomas and can contribute to its aggressiveness and increase the likelihood of recurrence. For U87-MG cells following treatment with L-NIL, the expression level of CD70 slightly decreased compared to the control for both the lower and higher doses, though neither treatment was statistically significantly (FIG. 13). The results indicate that L-NIL may confer a marginal benefit for in treating the subset of glioblastoma that is modeled by the U87-MG cell line, when considering CD70 as the predictive marker.

Both iNOS and nNOS can be overexpressed in glioblastomas and can contribute to its aggressiveness and increase the likelihood of recurrence. For U87-MG cells following treatment with L-NIL, the expression level of iNOS was slightly elevated at the lower dose and unchanged at the higher dose compared to the control treatment (FIG. 14), though neither result was statistically significantly. The expression level of nNOS, however, was reduced at both the lower dose and higher dose of L-NIL following treatment compared to the control treatment (FIG. 15), though neither result was statistically significantly. The results indicate that L-NIL may confer a marginal benefit for in treating the subset of glioblastoma that is modeled by the U87-MG cell line, when considering iNOS/nNOS as the predictive marker.

In xenografted mice, following treatment with L-NIL, mice showed a significantly smaller average tumor size compared the vehicle when administered 10 mg/kg (FIGS. 16-17).

NO takes part in the regulation of many physiological and pathological functions, which may include the development of glioblastoma and/or neuroblastoma. The results presented herein show that the inhibition of NO production in U87-MG cells (a human glioblastoma cell line), as well as the inhibition of tumor growth in U87-MG-xenografted mice by pharmacological intervention with the selective iNOS inhibitor L-NIL can effectively suppress the glioblastoma proliferation and growth, evidenced by significantly reduced levels total nitrate and nitrite content, 3-Ntyr, CD70, as well as reduced tumor growth in vivo, compared to the control.

Without being bound by any particular theory, it is believed that NO production may play a significant role in the development and progression of glioblastoma and/or neuroblastoma, among others. The effects of NO production may depend on NO concentration, exposure time, tumor type, microenvironment, and the like, however, high levels of NO (O(μM)) are suspected to cause DNA damage, and researchers have found that overexpression of NOS isoforms and excessive NO levels may play a role in the development and progression of glioblastoma and/or neuroblastoma and may even hinder the efficacy of conventional therapies used to treat these malignancies. It is believed that inflammation can induce NO-related carcinogenesis via induction of DNA damage, suppression of DNA repair enzymes, and PTMs including SNO, because during inflammation, reactive nitrogen and oxygen species are released by activated inflammatory cells, attacking neighboring epithelial and stromal cells, altering function, and initiating carcinogenesis. The events leading to the transition to a more aggressive tumor phenotype include activation or upregulation of pro-survival/pro-expansion effector proteins, such as, for example, NF-κB, phosphoinositide-3-kinase (PI3K), AKT, p300, among others.

Collectively, the results of this study suggest that inhibition of iNOS in a human glioblastoma model (i.e. U87-MG cells), both in vitro and in vivo, may suppress the malignancy of these cells. Because iNOS appears to be an important mediator of glioblastoma survival, proliferation, and carcinogenesis, suppression of iNOS using the novel NOS inhibitor compositions disclosed herein, e.g., L-NIL, may have a vast utility in treating, ameliorating, and/or reducing the severity of glioblastoma and/or neuroblastoma, as well as others cancer where overexpression of iNOS is implicated, e.g., gastrointestinal cancers, pancreatic cancer, sarcomas, ovarian cancers, cervical cancers, and the like. Moreover, because the other NOS isoforms, i.e., nNOS and eNOS, impart similar physiological effects, i.e., production of aberrant NO, the NOS inhibitor compositions disclosed herein, e.g., L-NIL, may be viable therapeutic options for pathologies wherein nNOS and/or eNOS are implicated.

### Example 111: L-NIL—Neuroblastoma

### Methods

Experiments will be performed in accordance with the methods disclosed in Example 15, but the one or more compositions will be prepared to comprise L-NIL and/or L-NIL hydrochloride instead of 3-Br-7-NI and/or a salt of 3-Br-7-NI.

### Example 112: L-NIL—Pancreatic Cancer

### Methods

Experiments will be performed in accordance with the methods disclosed in Example 4, but the one or more compositions will be prepared to comprise L-NIL and/or L-NIL hydrochloride instead of 7-NI and/or a salt of 7-NI.

### Example 113: L-NIL—Gastrointestinal Cancer

### Methods

Experiments will be performed in accordance with the methods disclosed in Example 5, but the one or more compositions will be prepared to comprise L-NIL and/or L-NIL hydrochloride instead of 7-NI and/or a salt of 7-NI.

### Example 114: L-NIL—Intestinal Adenoma

### Methods

Experiments will be performed in accordance with the methods disclosed in Example 6, but the one or more compositions will be prepared to comprise L-NIL and/or L-NIL hydrochloride instead of 7-NI and/or a salt of 7-NI.

### Example 115: L-NIL—Sarcoma

### Methods

Experiments will be performed in accordance with the methods disclosed in Example 7, but the one or more compositions will be prepared to comprise L-NIL and/or L-NIL hydrochloride instead of 7-NI and/or a salt of 7-NI.

### Example 116: L-NIL—Ovarian Cancer

### Methods

Experiments will be performed in accordance with the methods disclosed in Example 8, but the one or more compositions will be prepared to comprise L-NIL and/or L-NIL hydrochloride instead of 7-NI and/or a salt of 7-NI.

### Example 117: L-NIL—Cervical Cancer

### Methods

Experiments will be performed in accordance with the methods disclosed in Example 9, but the one or more compositions will be prepared to comprise L-NIL and/or L-NIL hydrochloride instead of 7-NI and/or a salt of 7-NI.

### Example 118: L-NIL—NO-Lung Cancer

### Methods

Experiments will be performed in accordance with the methods disclosed in Example 10, but the one or more compositions will be prepared to comprise L-NIL and/or L-NIL hydrochloride instead of 7-NI and/or a salt of 7-NI.

### Example 119: L-NIL—NSC-Lung Cancer

### Methods

Experiments will be performed in accordance with the methods disclosed in Example 11, but the one or more compositions will be prepared to comprise L-NIL and/or L-NIL hydrochloride instead of 7-NI and/or a salt of 7-NI.

### Example 120: L-NIL—m-HNSCC

### Methods

Experiments will be performed in accordance with the methods disclosed in Example 12, but the one or more compositions will be prepared to comprise L-NIL and/or L-NIL hydrochloride instead of 7-NI and/or a salt of 7-NI.

### Example 121: L-NIL—s-SCC

### Methods

Experiments will be performed in accordance with the methods disclosed in Example 13, but the one or more compositions will be prepared to comprise L-NIL and/or L-NIL hydrochloride instead of 7-NI and/or a salt of 7-NI.

### Example 122: 1400W—Glioblastoma

### Methods

Experiments will be performed in accordance with the methods disclosed in Example 14, but the one or more compositions will be prepared to comprise 1400W and/or 1400W dihydrochloride instead of 3-Br-7-NI and/or a salt of 3-Br-7-NI.

### Example 123: 1400W—Neuroblastoma

### Methods

Experiments will be performed in accordance with the methods disclosed in Example 15, but the one or more compositions will be prepared to comprise 1400W and/or 1400W dihydrochloride instead of 3-Br-7-NI and/or a salt of 3-Br-7-NI.

### Example 124: 1400W—Pancreatic Cancer

### Methods

Experiments will be performed in accordance with the methods disclosed in Example 4, but the one or more compositions will be prepared to comprise 1400W and/or 1400W dihydrochloride instead of 7-NI and/or a salt of 7-NI.

### Example 125: 1400W—Gastrointestinal Cancer

### Methods

Experiments will be performed in accordance with the methods disclosed in Example 5, but the one or more compositions will be prepared to comprise 1400W and/or 1400W dihydrochloride instead of 7-NI and/or a salt of 7-NI.

### Example 126: 1400W—Intestinal Adenoma

### Methods

Experiments will be performed in accordance with the methods disclosed in Example 6, but the one or more compositions will be prepared to comprise 1400W and/or 1400W dihydrochloride instead of 7-NI and/or a salt of 7-NI.

### Example 127: 1400W—Sarcoma

### Methods

Experiments will be performed in accordance with the methods disclosed in Example 7, but the one or more compositions will be prepared to comprise 1400W and/or 1400W dihydrochloride instead of 7-NI and/or a salt of 7-NI.

### Example 128: 1400W—Ovarian Cancer

### Methods

Experiments will be performed in accordance with the methods disclosed in Example 8, but the one or more compositions will be prepared to comprise 1400W and/or 1400W dihydrochloride instead of 7-NI and/or a salt of 7-NI.

### Example 129: 1400W—Cervical Cancer

### Methods

Experiments will be performed in accordance with the methods disclosed in Example 9, but the one or more compositions will be prepared to comprise 1400W and/or 1400W dihydrochloride instead of 7-NI and/or a salt of 7-NI.

### Example 130: 1400W—NO-Lung Cancer

### Methods

Experiments will be performed in accordance with the methods disclosed in Example 10, but the one or more compositions will be prepared to comprise 1400W and/or 1400W dihydrochloride instead of 7-NI and/or a salt of 7-NI.

### Example 131: 1400W—NSC-Lung Cancer

### Methods

Experiments will be performed in accordance with the methods disclosed in Example 11, but the one or more compositions will be prepared to comprise 1400W and/or 1400W dihydrochloride instead of 7-NI and/or a salt of 7-NI.

### Example 132: 1400W—m-HNSCC

### Methods

Experiments will be performed in accordance with the methods disclosed in Example 12, but the one or more compositions will be prepared to comprise 1400W and/or 1400W dihydrochloride instead of 7-NI and/or a salt of 7-NI.

### Example 133: 1400W—s-SCC

### Methods

Experiments will be performed in accordance with the methods disclosed in Example 13, but the one or more compositions will be prepared to comprise 1400W and/or 1400W dihydrochloride instead of 7-NI and/or a salt of 7-NI.

### Example 134: OKN-007—Glioblastoma

### Methods

Experiments will be performed in accordance with the methods disclosed in Example 14, but the one or more compositions will be prepared to comprise OKN-007 and/or OKN-007 sodium instead of 3-Br-7-NI and/or a salt of 3-Br-7-NI.

### Example 135: OKN-007—Neuroblastoma

### Methods

Experiments will be performed in accordance with the methods disclosed in Example 15, but the one or more compositions will be prepared to comprise OKN-007 and/or OKN-007 sodium instead of 3-Br-7-NI and/or a salt of 3-Br-7-NI.

### Example 136: OKN-007—Pancreatic Cancer

### Methods

Experiments will be performed in accordance with the methods disclosed in Example 4, but the one or more compositions will be prepared to comprise OKN-007 and/or OKN-007 sodium instead of 7-NI and/or a salt of 7-NI.

### Example 137: OKN-007—Gastrointestinal Cancer

### Methods

Experiments will be performed in accordance with the methods disclosed in Example 5, but the one or more compositions will be prepared to comprise OKN-007 and/or OKN-007 sodium instead of 7-NI and/or a salt of 7-NI.

### Example 138: OKN-007—Intestinal Adenoma

### Methods

Experiments will be performed in accordance with the methods disclosed in Example 6, but the one or more compositions will be prepared to comprise OKN-007 and/or OKN-007 sodium instead of 7-NI and/or a salt of 7-NI.

### Example 139: OKN-007—Sarcoma

### Methods

Experiments will be performed in accordance with the methods disclosed in Example 7, but the one or more compositions will be prepared to comprise OKN-007 and/or OKN-007 sodium instead of 7-NI and/or a salt of 7-NI.

### Example 140: OKN-007—Ovarian Cancer

### Methods

Experiments will be performed in accordance with the methods disclosed in Example 8, but the one or more compositions will be prepared to comprise OKN-007 and/or OKN-007 sodium instead of 7-NI and/or a salt of 7-NI.

### Example 141: OKN-007—Cervical Cancer

### Methods

Experiments will be performed in accordance with the methods disclosed in Example 9, but the one or more compositions will be prepared to comprise OKN-007 and/or OKN-007 sodium instead of 7-NI and/or a salt of 7-NI.

### Example 142: OKN-007—NO-Lung Cancer

### Methods

Experiments will be performed in accordance with the methods disclosed in Example 10, but the one or more compositions will be prepared to comprise OKN-007 and/or OKN-007 sodium instead of 7-NI and/or a salt of 7-NI.

### Example 143: OKN-007—NSC-Lung Cancer

### Methods

Experiments will be performed in accordance with the methods disclosed in Example 11, but the one or more compositions will be prepared to comprise OKN-007 and/or OKN-007 sodium instead of 7-NI and/or a salt of 7-NI.

### Example 144: OKN-007—m-HNSCC

### Methods

Experiments will be performed in accordance with the methods disclosed in Example 12, but the one or more compositions will be prepared to comprise OKN-007 and/or OKN-007 sodium instead of 7-NI and/or a salt of 7-NI.

### Example 145: OKN-007—s-SCC

### Methods

Experiments will be performed in accordance with the methods disclosed in Example 13, but the one or more compositions will be prepared to comprise OKN-007 and/or OKN-007 sodium instead of 7-NI and/or a salt of 7-NI.

### Example 146: BYK-191023—Glioblastoma

### Methods

Experiments will be performed in accordance with the methods disclosed in Example 14, but the one or more compositions will be prepared to comprise BYK-191023 and/or BYK-191023 dihydrochloride instead of 3-Br-7-NI and/or a salt of 3-Br-7-NI.

### Example 147: BYK-191023—Neuroblastoma

### Methods

Experiments will be performed in accordance with the methods disclosed in Example 15, but the one or more compositions will be prepared to comprise BYK-191023 and/or BYK-191023 dihydrochloride instead of 3-Br-7-NI and/or a salt of 3-Br-7-NI.

### Example 148: BYK-191023—Pancreatic Cancer

### Methods

Experiments will be performed in accordance with the methods disclosed in Example 4, but the one or more compositions will be prepared to comprise BYK-191023 and/or BYK-191023 dihydrochloride instead of 7-NI and/or a salt of 7-NI.

### Example 149: BYK-191023—Gastrointestinal Cancer

### Methods

Experiments will be performed in accordance with the methods disclosed in Example 5, but the one or more compositions will be prepared to comprise BYK-191023 and/or BYK-191023 dihydrochloride instead of 7-NI and/or a salt of 7-NI.

### Example 150: BYK-191023—Intestinal Adenoma

### Methods

Experiments will be performed in accordance with the methods disclosed in Example 6, but the one or more compositions will be prepared to comprise BYK-191023 and/or BYK-191023 dihydrochloride instead of 7-NI and/or a salt of 7-NI.

### Example 151: BYK-191023—Sarcoma

### Methods

Experiments will be performed in accordance with the methods disclosed in Example 7, but the one or more compositions will be prepared to comprise BYK-191023 and/or BYK-191023 dihydrochloride instead of 7-NI and/or a salt of 7-NI.

### Example 152: BYK-191023—Ovarian Cancer

### Methods

Experiments will be performed in accordance with the methods disclosed in Example 8, but the one or more compositions will be prepared to comprise BYK-191023 and/or BYK-191023 dihydrochloride instead of 7-NI and/or a salt of 7-NI.

### Example 153: BYK-191023—Cervical Cancer

### Methods

Experiments will be performed in accordance with the methods disclosed in Example 9, but the one or more compositions will be prepared to comprise BYK-191023 and/or BYK-191023 dihydrochloride instead of 7-NI and/or a salt of 7-NI.

### Example 154: BYK-191023—NO-Lung Cancer

### Methods

Experiments will be performed in accordance with the methods disclosed in Example 10, but the one or more compositions will be prepared to comprise BYK-191023 and/or BYK-191023 dihydrochloride instead of 7-NI and/or a salt of 7-NI.

### Example 155: BYK-191023—NSC-Lung Cancer

### Methods

Experiments will be performed in accordance with the methods disclosed in Example 11, but the one or more compositions will be prepared to comprise BYK-191023 and/or BYK-191023 dihydrochloride instead of 7-NI and/or a salt of 7-NI.

### Example 156: BYK-191023—m-HNSCC

### Methods

Experiments will be performed in accordance with the methods disclosed in Example 12, but the one or more compositions will be prepared to comprise BYK-191023 and/or BYK-191023 dihydrochloride instead of 7-NI and/or a salt of 7-NI.

### Example 157: BYK-191023—s-SCC

### Methods

Experiments will be performed in accordance with the methods disclosed in Example 13, but the one or more compositions will be prepared to comprise BYK-191023 and/or BYK-191023 dihydrochloride instead of 7-NI and/or a salt of 7-NI.

### Example 158: CM544—Glioblastoma

### Methods

Experiments will be performed in accordance with the methods disclosed in Example 14, but the one or more compositions will be prepared to comprise CM544 and/or CM544 dihydrochloride instead of 3-Br-7-NI and/or a salt of 3-Br-7-NI.

### Example 159: CM544—Neuroblastoma

### Methods

Experiments will be performed in accordance with the methods disclosed in Example 15, but the one or more compositions will be prepared to comprise CM544 and/or CM544 dihydrochloride instead of 3-Br-7-NI and/or a salt of 3-Br-7-NI.

### Example 160: CM544—Pancreatic Cancer

### Methods

Experiments will be performed in accordance with the methods disclosed in Example 4, but the one or more compositions will be prepared to comprise CM544 and/or CM544 dihydrochloride instead of 7-NI and/or a salt of 7-NI.

### Example 161: CM544—Gastrointestinal Cancer

### Methods

Experiments will be performed in accordance with the methods disclosed in Example 5, but the one or more compositions will be prepared to comprise CM544 and/or CM544 dihydrochloride instead of 7-NI and/or a salt of 7-NI.

### Example 162: CM544—Intestinal Adenoma

### Methods

Experiments will be performed in accordance with the methods disclosed in Example 6, but the one or more compositions will be prepared to comprise CM544 and/or CM544 dihydrochloride instead of 7-NI and/or a salt of 7-NI.

### Example 163: CM544—Sarcoma

### Methods

Experiments will be performed in accordance with the methods disclosed in Example 7, but the one or more compositions will be prepared to comprise CM544 and/or CM544 dihydrochloride instead of 7-NI and/or a salt of 7-NI.

### Example 164: CM544—Ovarian Cancer

### Methods

Experiments will be performed in accordance with the methods disclosed in Example 8, but the one or more compositions will be prepared to comprise CM544 and/or CM544 dihydrochloride instead of 7-NI and/or a salt of 7-NI.

### Example 165: CM544—Cervical Cancer

### Methods

Experiments will be performed in accordance with the methods disclosed in Example 9, but the one or more compositions will be prepared to comprise CM544 and/or CM544 dihydrochloride instead of 7-NI and/or a salt of 7-NI.

### Example 166: CM544—NO-Lung Cancer

### Methods

Experiments will be performed in accordance with the methods disclosed in Example 10, but the one or more compositions will be prepared to comprise CM544 and/or CM544 dihydrochloride instead of 7-NI and/or a salt of 7-NI.

### Example 167: CM544—NSC-Lung Cancer

### Methods

Experiments will be performed in accordance with the methods disclosed in Example 11, but the one or more compositions will be prepared to comprise CM544 and/or CM544 dihydrochloride instead of 7-NI and/or a salt of 7-NI.

### Example 168: CM544—m-HNSCC

### Methods

Experiments will be performed in accordance with the methods disclosed in Example 12, but the one or more compositions will be prepared to comprise CM544 and/or CM544 dihydrochloride instead of 7-NI and/or a salt of 7-NI.

### Example 169: CM544—s-SCC

### Methods

Experiments will be performed in accordance with the methods disclosed in Example 13, but the one or more compositions will be prepared to comprise CM544 and/or CM544 dihydrochloride instead of 7-NI and/or a salt of 7-NI.

### Example 170: Combination—Glioblastoma

### Methods

Experiments will be performed in accordance with the methods disclosed in Example 14, but the one or more compositions will be prepared to comprise a combination of at least two compounds selected from the group consisting of 7-NI, a salt of 7-NI, 3-Br-7-NI, a salt of 3-Br-7-NI, L-NPA, L-NPA hydrochloride, L-NIO, L-NIO dihydrochloride, ARL-17477, and/or ARL-17477 dihydrochloride, L-NNA, a salt of L-NNA, L-NAME, L-NAME hydrochloride, L-NMMA, L-NMMA acetate, GW-274150, GW-274150 phosphate, L-NIL, L-NIL hydrochloride, 1400W, 1400W dihydrochloride, OKN-007, OKN-007 sodium, BYK-191023, BYK-191023 dihydrochloride, CM544, and/or CM544 dihydrochloride, as well as any salt of the foregoing not expressly listed, instead of 3-Br-7-NI and/or a salt of 3-Br-7-NI. For combinations, each compound will be formulated with a dose in the range of 0.1 μg/kg to 100 mg/kg.

In a particular example, in vitro and in vivo experiments were performed using U87-MG cells and U87-MG-xenografted mice, respectively, as a glioblastoma model. The same methods that were utilized in Examples 3 and 110 were utilized in Example 170. The difference in Example 94 compared to Examples 3 and 110, is that a combination of 7-NI and L-NIL were administered concurrently in vitro and in vivo.

For the in vivo experiments, an initial experiment was performed with two treatment groups of mice, the treatment groups were: control (n=10) and 80 mg/kg of 7-NI and 10 mg/kg of L-NIL (n=15). Treatments were administered intraperitoneally daily, for 8 days. Treatment started after five days from the injection with U87-MG cells. After one month, all mice were euthanized, and tumors were harvested for ex vivo analysis and histology. Tumor volume was measured using calipers and was calculated as (length*width\hat{ }2)/2. The tumor volume of all mice were tabulated, as well the best 4-5 mice in terms of treatment efficacy (smallest tumor volume).

### Results

In the colony assay, the combinations, at both doses, significantly impacted colony growth compared to both the control and the vehicle (0.1% DMSO) (FIG. 9). Compared to either 7-NI or L-NIL administered alone, both doses of the combinations reduced colony growth more than 7-NI and L-NIL administered alone (FIG. 9), though not statistically significant. These results indicate that the maximum effect, i.e., a decrease in the number of colonies, can be achieved by administering two compounds that individually target the activity of both nNOS (7-NI) and iNOS (L-NIL) compared to either compound used individually. Accordingly, because the colony growth assay can be used as a metric to predict success of treating glioblastoma, the results suggest that the combination of 7-NI and L-NIL may be effective at treating glioblastoma.

In the Griess assay, the combination treatment resulted in a reduction of nitrite and nitrate content in U87-MG cells compared to the vehicle (0.1% DMSO) for both doses of combinations evaluated (FIG. 11). For the low dose combination, the nitrite and nitrate content was reduced following treatment, though not statistically significant compared to the vehicle (0.1% DMSO), whereas for the high dose combination the nitrite and nitrate content was significantly reduced following treatment compared the vehicle (0.1% DMSO) (FIG. 11). Notably, of all of treatments evaluated, the high dose combination conferred the largest reduction of nitrite and nitrate content in U87-MG cells. Because the combination contains an iNOS-specific inhibitor (L-NIL) and nNOS-specific inhibitor (7-NI), these results indicate that the observed decrease in nitrite and nitrate content may occur because of global, simultaneous suppression of multiple NOS isoforms.

In xenografted mice, following treatment with the combination (80 mg/kg 7-NI and 10 mg/kg L-NIL), the combination conferred the most beneficial result, as the tumor volume was smallest for the combination—this result was statistically significant compared to the vehicle (0.1% DMSO) and L-NIL administered alone (10 mg/kg), and was not statistically significant compared to 7-NI administered alone (80 mg/kg) (FIG. 16). However, when the best 4-5 examples in the same groups were considered, the results indicate that the combination was significantly better, i.e., resulted in the smallest tumor, compared to any other condition evaluated (FIG. 17). These results indicate that while the combination provides the greatest treatment option, a subset within that group may have a better treatment outcome, i.e., less tumor growth, than an individual treatment by itself.

NO takes part in the regulation of many physiological and pathological functions, which may include the development of glioblastoma and/or neuroblastoma. The results presented herein show that the inhibition of NO production in U87-MG cells (a human glioblastoma cell line), as well as the inhibition of tumor growth in U87-MG-xenografted mice by pharmacological intervention with a combination with a selective iNOS inhibitor (L-NIL) and a selective nNOS inhibitor (7-NI) can effectively suppress the glioblastoma proliferation and growth, evidenced by significantly reduced levels total nitrate and nitrite content, 3-Ntyr, CD70, as well as reduced tumor growth in vivo, compared to the control and individual treatments alone.

Without being bound by any particular theory, it is believed that NO production may play a significant role in the development and progression of glioblastoma and/or neuroblastoma, among others. The effects of NO production may depend on NO concentration, exposure time, tumor type, microenvironment, and the like, however, high levels of NO (O(μM)) are suspected to cause DNA damage, and researchers have found that overexpression of NOS isoforms and excessive NO levels may play a role in the development and progression of glioblastoma and/or neuroblastoma and may even hinder the efficacy of conventional therapies used to treat these malignancies. It is believed that inflammation can induce NO-related carcinogenesis via induction of DNA damage, suppression of DNA repair enzymes, and PTMs including SNO, because during inflammation, reactive nitrogen and oxygen species are released by activated inflammatory cells, attacking neighboring epithelial and stromal cells, altering function, and initiating carcinogenesis. The events leading to the transition to a more aggressive tumor phenotype include activation or upregulation of pro-survival/pro-expansion effector proteins, such as, for example, NF-κB, phosphoinositide-3-kinase (PI3K), AKT, p300, among others.

Collectively, the results of this study suggest providing a combination of compounds that inhibit both nNOS and iNOS in a human glioblastoma model (i.e. U87-MG cells), both in vitro and in vivo, may suppress the malignancy of these cells. Because nNOS and iNOS both appear to be important mediators of glioblastoma survival, proliferation, and carcinogenesis, suppression of both of these isoforms, may have a vast utility in treating, ameliorating, and/or reducing the severity of glioblastoma and/or neuroblastoma, as well as others cancer where overexpression of nNOS, iNOS, or both nNOS and iNOS are implicated, e.g., gastrointestinal cancers, pancreatic cancer, sarcomas, ovarian cancers, cervical cancers, and the like. Moreover, because the other NOS isoforms, i.e., eNOS, impart similar physiological effects, i.e., production of aberrant NO, the NOS inhibitor compositions disclosed herein, e.g., L-NIL and 7-NI, may be viable therapeutic options for pathologies where eNOS is implicated.

### Example 171: Combination—Neuroblastoma

### Methods

Experiments will be performed in accordance with the methods disclosed in Example 15, but the one or more compositions will be prepared to comprise a combination of at least two compounds selected from the group consisting of 7-NI, a salt of 7-NI, 3-Br-7-NI, a salt of 3-Br-7-NI, L-NPA, L-NPA hydrochloride, L-NIO, L-NIO dihydrochloride, ARL-17477, and/or ARL-17477 dihydrochloride, L-NNA, a salt of L-NNA, L-NAME, L-NAME hydrochloride, L-NMMA, L-NMMA acetate, GW-274150, GW-274150 phosphate, L-NIL, L-NIL hydrochloride, 1400W, 1400W dihydrochloride, OKN-007, OKN-007 sodium, BYK-191023, BYK-191023 dihydrochloride, CM544, and/or CM544 dihydrochloride, as well as any salt of the foregoing not expressly listed, instead of 3-Br-7-NI and/or a salt of 3-Br-7-NI. For combinations, each compound will be formulated with a dose in the range of 0.1 μg/kg to 100 mg/kg.

### Example 172: Combination—Pancreatic Cancer

### Methods

Experiments will be performed in accordance with the methods disclosed in Example 4, but the one or more compositions will be prepared to comprise a combination of at least two compounds selected from the group consisting of 7-NI, a salt of 7-NI, 3-Br-7-NI, a salt of 3-Br-7-NI, L-NPA, L-NPA hydrochloride, L-NIO, L-NIO dihydrochloride, ARL-17477, and/or ARL-17477 dihydrochloride, L-NNA, a salt of L-NNA, L-NAME, L-NAME hydrochloride, L-NMMA, L-NMMA acetate, GW-274150, GW-274150 phosphate, L-NIL, L-NIL hydrochloride, 1400W, 1400W dihydrochloride, OKN-007, OKN-007 sodium, BYK-191023, BYK-191023 dihydrochloride, CM544, and/or CM544 dihydrochloride, as well as any salt of the foregoing not expressly listed, instead of 3-Br-7-NI and/or a salt of 3-Br-7-NI. For combinations, each compound will be formulated with a dose in the range of 0.1 μg/kg to 100 mg/kg.

### Example 173: Combination—Gastrointestinal Cancer

### Methods

Experiments will be performed in accordance with the methods disclosed in Example 5, but the one or more compositions will be prepared to comprise a combination of at least two compounds selected from the group consisting of 7-NI, a salt of 7-NI, 3-Br-7-NI, a salt of 3-Br-7-NI, L-NPA, L-NPA hydrochloride, L-NIO, L-NIO dihydrochloride, ARL-17477, and/or ARL-17477 dihydrochloride, L-NNA, a salt of L-NNA, L-NAME, L-NAME hydrochloride, L-NMMA, L-NMMA acetate, GW-274150, GW-274150 phosphate, L-NIL, L-NIL hydrochloride, 1400W, 1400W dihydrochloride, OKN-007, OKN-007 sodium, BYK-191023, BYK-191023 dihydrochloride, CM544, and/or CM544 dihydrochloride, as well as any salt of the foregoing not expressly listed, instead of 3-Br-7-NI and/or a salt of 3-Br-7-NI. For combinations, each compound will be formulated with a dose in the range of 0.1 μg/kg to 100 mg/kg.

### Example 174: Combination—Intestinal Adenoma

### Methods

Experiments will be performed in accordance with the methods disclosed in Example 6, but the one or more compositions will be prepared to comprise a combination of at least two compounds selected from the group consisting of 7-NI, a salt of 7-NI, 3-Br-7-NI, a salt of 3-Br-7-NI, L-NPA, L-NPA hydrochloride, L-NIO, L-NIO dihydrochloride, ARL-17477, and/or ARL-17477 dihydrochloride, L-NNA, a salt of L-NNA, L-NAME, L-NAME hydrochloride, L-NMMA, L-NMMA acetate, GW-274150, GW-274150 phosphate, L-NIL, L-NIL hydrochloride, 1400W, 1400W dihydrochloride, OKN-007, OKN-007 sodium, BYK-191023, BYK-191023 dihydrochloride, CM544, and/or CM544 dihydrochloride, as well as any salt of the foregoing not expressly listed, instead of 3-Br-7-NI and/or a salt of 3-Br-7-NI. For combinations, each compound will be formulated with a dose in the range of 0.1 μg/kg to 100 mg/kg.

### Example 175: Combination—Sarcoma

### Methods

Experiments will be performed in accordance with the methods disclosed in Example 7, but the one or more compositions will be prepared to comprise a combination of at least two compounds selected from the group consisting of 7-NI, a salt of 7-NI, 3-Br-7-NI, a salt of 3-Br-7-NI, L-NPA, L-NPA hydrochloride, L-NIO, L-NIO dihydrochloride, ARL-17477, and/or ARL-17477 dihydrochloride, L-NNA, a salt of L-NNA, L-NAME, L-NAME hydrochloride, L-NMMA, L-NMMA acetate, GW-274150, GW-274150 phosphate, L-NIL, L-NIL hydrochloride, 1400W, 1400W dihydrochloride, OKN-007, OKN-007 sodium, BYK-191023, BYK-191023 dihydrochloride, CM544, and/or CM544 dihydrochloride, as well as any salt of the foregoing not expressly listed, instead of 3-Br-7-NI and/or a salt of 3-Br-7-NI. For combinations, each compound will be formulated with a dose in the range of 0.1 μg/kg to 100 mg/kg.

### Example 176: Combination—Ovarian Cancer

### Methods

Experiments will be performed in accordance with the methods disclosed in Example 8, but the one or more compositions will be prepared to comprise a combination of at least two compounds selected from the group consisting of 7-NI, a salt of 7-NI, 3-Br-7-NI, a salt of 3-Br-7-NI, L-NPA, L-NPA hydrochloride, L-NIO, L-NIO dihydrochloride, ARL-17477, and/or ARL-17477 dihydrochloride, L-NNA, a salt of L-NNA, L-NAME, L-NAME hydrochloride, L-NMMA, L-NMMA acetate, GW-274150, GW-274150 phosphate, L-NIL, L-NIL hydrochloride, 1400W, 1400W dihydrochloride, OKN-007, OKN-007 sodium, BYK-191023, BYK-191023 dihydrochloride, CM544, and/or CM544 dihydrochloride, as well as any salt of the foregoing not expressly listed, instead of 3-Br-7-NI and/or a salt of 3-Br-7-NI. For combinations, each compound will be formulated with a dose in the range of 0.1 μg/kg to 100 mg/kg.

### Example 177: Combination—Cervical Cancer

### Methods

Experiments will be performed in accordance with the methods disclosed in Example 9, but the one or more compositions will be prepared to comprise a combination of at least two compounds selected from the group consisting of 7-NI, a salt of 7-NI, 3-Br-7-NI, a salt of 3-Br-7-NI, L-NPA, L-NPA hydrochloride, L-NIO, L-NIO dihydrochloride, ARL-17477, and/or ARL-17477 dihydrochloride, L-NNA, a salt of L-NNA, L-NAME, L-NAME hydrochloride, L-NMMA, L-NMMA acetate, GW-274150, GW-274150 phosphate, L-NIL, L-NIL hydrochloride, 1400W, 1400W dihydrochloride, OKN-007, OKN-007 sodium, BYK-191023, BYK-191023 dihydrochloride, CM544, and/or CM544 dihydrochloride, as well as any salt of the foregoing not expressly listed, instead of 3-Br-7-NI and/or a salt of 3-Br-7-NI. For combinations, each compound will be formulated with a dose in the range of 0.1 μg/kg to 100 mg/kg.

### Example 178: Combination—NO-Lung Cancer

### Methods

Experiments will be performed in accordance with the methods disclosed in Example 10, but the one or more compositions will be prepared to comprise a combination of at least two compounds selected from the group consisting of 7-NI, a salt of 7-NI, 3-Br-7-NI, a salt of 3-Br-7-NI, L-NPA, L-NPA hydrochloride, L-NIO, L-NIO dihydrochloride, ARL-17477, and/or ARL-17477 dihydrochloride, L-NNA, a salt of L-NNA, L-NAME, L-NAME hydrochloride, L-NMMA, L-NMMA acetate, GW-274150, GW-274150 phosphate, L-NIL, L-NIL hydrochloride, 1400W, 1400W dihydrochloride, OKN-007, OKN-007 sodium, BYK-191023, BYK-191023 dihydrochloride, CM544, and/or CM544 dihydrochloride, as well as any salt of the foregoing not expressly listed, instead of 3-Br-7-NI and/or a salt of 3-Br-7-NI. For combinations, each compound will be formulated with a dose in the range of 0.1 μg/kg to 100 mg/kg.

### Example 179: Combination—NSC-Lung Cancer

### Methods

Experiments will be performed in accordance with the methods disclosed in Example 11, but the one or more compositions will be prepared to comprise a combination of at least two compounds selected from the group consisting of 7-NI, a salt of 7-NI, 3-Br-7-NI, a salt of 3-Br-7-NI, L-NPA, L-NPA hydrochloride, L-NIO, L-NIO dihydrochloride, ARL-17477, and/or ARL-17477 dihydrochloride, L-NNA, a salt of L-NNA, L-NAME, L-NAME hydrochloride, L-NMMA, L-NMMA acetate, GW-274150, GW-274150 phosphate, L-NIL, L-NIL hydrochloride, 1400W, 1400W dihydrochloride, OKN-007, OKN-007 sodium, BYK-191023, BYK-191023 dihydrochloride, CM544, and/or CM544 dihydrochloride, as well as any salt of the foregoing not expressly listed, instead of 3-Br-7-NI and/or a salt of 3-Br-7-NI. For combinations, each compound will be formulated with a dose in the range of 0.1 μg/kg to 100 mg/kg.

### Example 180: Combination—m-HNSCC

### Methods

Experiments will be performed in accordance with the methods disclosed in Example 12, but the one or more compositions will be prepared to comprise a combination of at least two compounds selected from the group consisting of 7-NI, a salt of 7-NI, 3-Br-7-NI, a salt of 3-Br-7-NI, L-NPA, L-NPA hydrochloride, L-NIO, L-NIO dihydrochloride, ARL-17477, and/or ARL-17477 dihydrochloride, L-NNA, a salt of L-NNA, L-NAME, L-NAME hydrochloride, L-NMMA, L-NMMA acetate, GW-274150, GW-274150 phosphate, L-NIL, L-NIL hydrochloride, 1400W, 1400W dihydrochloride, OKN-007, OKN-007 sodium, BYK-191023, BYK-191023 dihydrochloride, CM544, and/or CM544 dihydrochloride, as well as any salt of the foregoing not expressly listed, instead of 3-Br-7-NI and/or a salt of 3-Br-7-NI. For combinations, each compound will be formulated with a dose in the range of 0.1 μg/kg to 100 mg/kg.

### Example 181: Combination—s-SCC

### Methods

Experiments will be performed in accordance with the methods disclosed in Example 13, but the one or more compositions will be prepared to comprise a combination of at least two compounds selected from the group consisting of 7-NI, a salt of 7-NI, 3-Br-7-NI, a salt of 3-Br-7-NI, L-NPA, L-NPA hydrochloride, L-NIO, L-NIO dihydrochloride, ARL-17477, and/or ARL-17477 dihydrochloride, L-NNA, a salt of L-NNA, L-NAME, L-NAME hydrochloride, L-NMMA, L-NMMA acetate, GW-274150, GW-274150 phosphate, L-NIL, L-NIL hydrochloride, 1400W, 1400W dihydrochloride, OKN-007, OKN-007 sodium, BYK-191023, BYK-191023 dihydrochloride, CM544, and/or CM544 dihydrochloride, as well as any salt of the foregoing not expressly listed, instead of 3-Br-7-NI and/or a salt of 3-Br-7-NI. For combinations, each compound will be formulated with a dose in the range of 0.1 μg/kg to 100 mg/kg.

The present disclosure is not to be limited in terms of the particular embodiments described in this application, which are intended as illustrations of various aspects of the present disclosure. Many modifications and variations can be made without departing from its spirit and scope. Functionally equivalent methods and apparatuses within the scope of the disclosure, in addition to those enumerated herein, are possible from the foregoing descriptions. Such modifications and variations are intended to fall within the scope of the appended claims. The terminology used herein is for the purpose of describing particular embodiments only and is not intended to be limiting unless otherwise set forth herein.

1.-41. (canceled)

42. A method of treating cancer in a subject which a beneficial clinical effect is achieved by reduction in neuronal nitric oxide synthase (nNOS) activity, the method comprises administering to the subject a pharmaceutical composition comprising an active agent which reduces or inhibits nNOS enzymatic activity, wherein the cancer is glioblastoma or pancreatic cancer.

43. The method of claim 42, wherein the active agent is selected from the group consisting of 7-nitroindazole (“7-NI”), a salt of 7-NI, 3-bromo-7-nitroindazole (“3-Br-7-NI”), a salt of 3-Br-7-NI, and any combination thereof.

44. The method of claim 43, wherein said agent is 7-nitro-indazole (7-NI), a salt of 7-NI, and/or a metal complex of 7-NI.

45. The method according to claim 44, wherein the active agent is 7-NI.

46. A method of treating melanoma in a subject, the method comprises administering to the subject a pharmaceutical composition comprising an active agent selected from 7-nitro-indazole (7-NI), a salt of 7-NI, and/or a metal complex of 7-NI.

47. The method according to claim 46, wherein the active agent is 7-NI.

48. A method of treating cancer in a subject, the method comprises administering to the subject a pharmaceutical composition comprising an active agent selected from 7-nitro-indazole (7-NI), a salt of 7-NI, and/or a metal complex of 7-NI, wherein said cancer is glioblastoma or pancreatic cancer.

49. The method according to claim 48, wherein the cancer is glioblastoma and the active agent is 7-NI.

50. The method according to claim 48, wherein the cancer is pancreatic cancer and the active agent is 7-NI.


SEQUENCE LISTING (14 sequences)

SEQ ID NO: 1-2 - skipped (2 entries)

SEQ ID NO: 3 - RNA, 25 nt, synthetic construct
  misc_feature 1..25: note = SHANK3 siRNA
  source 1..25: mol_type = other RNA
cgatgataagcagtttgcaaagctt

SEQ ID NO: 4 - RNA, 25 nt, synthetic construct
  misc_feature 1..25: note = nNOS siRNA 1
  source 1..25: mol_type = other RNA
ccttgtccaacatgctcctagagat

SEQ ID NO: 5 - RNA, 25 nt, synthetic construct
  misc_feature 1..25: note = nNOS siRNA 2
  source 1..25: mol_type = other RNA
atctctaggagcatgttggacacgg

SEQ ID NO: 6 - RNA, 25 nt, synthetic construct
  misc_feature 1..25: note = nNOS siRNA 3
  source 1..25: mol_type = other RNA
ccaatgtcatttctgttcgtctctt

SEQ ID NO: 7 - RNA, 25 nt, synthetic construct
  misc_feature 1..25: note = nNOS siRNA 4
  source 1..25: mol_type = other RNA
aagagacgaacagaaatgacattgg

SEQ ID NO: 8 - RNA, 25 nt, synthetic construct
  misc_feature 1..25: note = nNOS siRNA 5
  source 1..25: mol_type = other RNA
ccggagacatcattcttgcggtcaa

SEQ ID NO: 9 - RNA, 70 nt, synthetic construct
  misc_feature 1..70: note = nNOS shRNA 1
  source 1..70: mol_type = other RNA
gtacctccccacatttatggcttgtttaagtcaagagcttaaacaagccataaatgtgggtttttggaaa

SEQ ID NO: 10 - RNA, 70 nt, synthetic construct
  misc_feature 1..70: note = nNOS shRNA 2
  source 1..70: mol_type = other RNA
agcttttccaaaaacccacatttatggcttgtttaagctcttgacttaaacaagccataaatgtggggag

SEQ ID NO: 11 - RNA, 60 nt, synthetic construct
  misc_feature 1..60: note = nNOS shRNA 3
  source 1..60: mol_type = other RNA
acctctcccaaagtgctgagattacaggtcaagagcctgtaatctcagcactttgggatt

SEQ ID NO: 12 - RNA, 60 nt, synthetic construct
  misc_feature 1..60: note = nNOS shRNA 4
  source 1..60: mol_type = other RNA
caaaaatcccaaagtgctgagattacaggctcttgacctgtaatctcagcactttgggag

SEQ ID NO: 13 - RNA, 70 nt, synthetic construct
  misc_feature 1..70: note = nNOS shRNA 5
  source 1..70: mol_type = other RNA
gtacctctccagttgtaacccacatttatgtcaagagcataaatgtgggttacaactggatttttggaaa

SEQ ID NO: 14 - RNA, 70 nt, synthetic construct
  misc_feature 1..70: note = nNOS shRNA 6
  source 1..70: mol_type = other RNA
agcttttccaaaaatccagttgtaacccacatttatgctcttgacataaatgtgggttacaactggagag